(19) United States
(12) Patent Application Publication
(10) Pub. No.: US 2023/0339970 A1
(43) Pub. Date: Oct. 26, 2023

(54) CASEIN KINASE 1 DELTA MODULATORS

(71) Applicant: Janssen Pharmaceutica NV, Beerse (BE)

(72) Inventors: Terry Patrick Lebold, San Diego, CA (US); Cathy Preville, San Diego, CA (US); Andrew V. Samant, Cardiff, CA (US); Brock T. Shireman, Poway, CA (US); Luke Edward Hanna, San Diego, CA (US); Mark Seierstad, Escondido, CA (US); Stefan McCarver, San Diego, CA (US); Dongpei Wu, San Diego, CA (US); Kelsey Patrick, San Diego, CA (US); Elizabeth Swift, San Diego, CA (US)

(21) Appl. No.: 18/026,607

(22) PCT Filed: Sep. 16, 2021
(86) PCT No.: PCT/IB2021/058445

§ 371 Date: Mar. 16, 2023

Related U.S. Application Data
(60) Provisional application No. 63/079,789, filed on Sep. 17, 2020.

(51) Int. Cl. C07D 498/20; C07D 471/04; C07D 471/08; C07D 487/04; C07D 498/04; C07D 498/10; C07D 519/00

(52) U.S. Cl. CPC C07D 498/20; C07D 471/04; C07D 471/08; C07D 487/04; C07D 498/04; C07D 498/10; C07D 519/00

(57) ABSTRACT

A compound of Formula (I), pharmaceutical compositions containing them, methods of making them, and methods of using them including methods for treating disease states, disorders, and conditions associated with casein kinase 1 delta (CSNK1D) modulation, such as those associated with mood/psychiatric disorders, neurodegenerative diseases, cancers, addiction and substance abuse disorders, pain, and metabolic diseases.

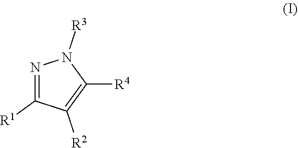

Wherein R¹, R², R³, and R⁴, are defined herein.

### FIELD OF THE INVENTION

The present invention is related to certain fused chemical entities having Casein kinase 1 delta (CSNK1D) modulating properties, pharmaceutical compositions comprising these chemical entities, chemical processes for preparing these chemical entities and their use in the treatment of diseases, disorders, or conditions.

### BACKGROUND OF THE INVENTION

Disruption of the circadian rhythm is a major hallmark in mood disorders. Dampened and phase-shifted temperature, activity, and hormonal rhythms are frequently reported in major depressive disorder (MDD) and bipolar disorder (Hickie, I. B., et al., Manipulating the sleep-wake cycle and circadian rhythms to improve clinical management of major depression. BMC Med, 2013. 11: p. 79; Germain, A. and D. J. Kupfer, Circadian rhythm disturbances in depression. Hum Psychopharmacol, 2008. 23(7): p. 571-85). Depression symptoms are also diurnal with the most severe symptoms occurring typically in the morning (Rusting, C. L. and R. J. Larsen, Diurnal patterns of unpleasant mood: associations with neuroticism, depression, and anxiety. J Pers, 1998. 66(1): p. 85-103), and depression is more prevalent in areas of the world that receive little sunlight for extended periods of time (Booker, J. M., et al., Seasonal depression and sleep disturbances in Alaska and Siberia: a pilot study. Arctic Med Res, 1991. Suppl: p. 281-4). One of the most common mood disorders is seasonal affective disorder (SAD), a syndrome where depressive symptoms occur only in the winter months when there are shorter days and a later dawn (Lam, R. W. and R. D. Levitan, Pathophysiology of seasonal affective disorder: a review. J Psychiatry Neurosci, 2000. 25(5): p. 469-80; Magnusson, A. and D. Boivin, Seasonal affective disorder: an overview. Chronobiol Int, 2003. 20(2): p. 189-207). Therefore, identifying mechanisms that correct these circadian disruptions may have the added therapeutic benefit of attenuating mood disorders.

Many circadian genes have been associated with mood disorders (Benedetti, F., et al., Influence of CLOCK gene polymorphism on circadian mood fluctuation and illness recurrence in bipolar depression. Am J Med Genet B Neuropsychiatr Genet, 2003. 123B(1): p. 23-6; Soria, V., et al., Differential association of circadian genes with mood disorders: CRY1 and NPAS2 are associated with unipolar major depression and CLOCK and VIP with bipolar disorder. Neuropsychopharmacology, 2010. 35(6): p. 1279-89). Pre-clinically, disruption of the suprachiasmatic nucleus (SCN) molecular clock by knocking down Bmal1 expression in the SCN resulted in a dampening and lengthening of SCN PER2:LUC rhythms in mice and lengthened wheel-running rhythms (Landgraf, D., et al., Genetic Disruption of Circadian Rhythms in the Suprachiasmatic Nucleus Causes Helplessness, Behavioral Despair, and Anxiety-like Behavior in Mice. Biol Psychiatry, 2016. 80(11): p. 827-835). Most notably, disruption of SCN molecular rhythms increased depression-like behavior in the learned helplessness and tail suspension tests (Ko, C. H. and J. S. Takahashi, Molecular components of the mammalian circadian clock. Hum Mol Genet, 2006. 15 Spec No 2: p. R271-7; Reppert, S. M. and D. R. Weaver, Molecular analysis of mammalian circadian rhythms. Annu Rev Physiol, 2001. 63: p. 647-76). Additionally, SCN BMAL1 knockdown increased anxiety-like behavior in the light/dark box. Together, these findings suggest that reduced amplitude and increased period of SCN molecular rhythms can cause increased depression and anxiety-like behavior.

The primary molecular clock that controls circadian rhythm is in the SCN in the hypothalamus and consists of a transcriptional feedback loop which cycles over the course of approximately 24 hours (Ko, C. H. and J. S. Takahashi, Molecular components of the mammalian circadian clock. Hum Mol Genet, 2006. 15 Spec No 2: p. R271-7; Reppert, S. M. and D. R. Weaver, Molecular analysis of mammalian circadian rhythms. Annu Rev Physiol, 2001. 63: p. 647-76). The major transcriptional activator consists of a dimer between the Circadian Locomotor Output Cycles Kaput Protein (CLOCK) and Brain and Muscle ARNT-like Protein 1 (BMAL1). This complex binds to the promoters of many genes including the Period (Per) and Cryptochrome (Cry) genes. CRY and PER proteins form a heterodimer in the cytoplasm and translocate into the nucleus where they repress the actions of CLOCK/BMAL1, thus creating a negative feedback loop whose timing is regulated by numerous kinases. Casein kinase 1 delta (CSNK1D) is known to modulate the various feedback loops of the internal canonical circadian clock by phosphorylating PER2. A previous report has demonstrated that two distinct CSNK1D inhibitors PF-670462 and PF-5006739 significantly lengthened circadian rhythms in both cellular reporter assays and in vivo locomotor activity of a variety of species (Wager Travis T. et al., Casein Kinase 1δ/ε Inhibitor PF-5006739 Attenuates Opioid Drug-Seeking Behavior, 2014 Dec. 17; 5(12): p. 1253-65). Period lengthening mediated by CSNK1D inhibition was accompanied by increased nuclear retention and localization of PER2 protein both in vitro and in vivo (Meng, Q. J., et al., Entrainment of disrupted circadian behavior through inhibition of casein kinase 1 (CK1) enzymes. Proc Natl Acad Sci USA, 2010. 107(34): p. 15240-5; Smyllie, N.J., et al., Visualizing and Quantifying Intracellular Behavior and Abundance of the Core Circadian Clock Protein PERIOD2. Curr Biol, 2016. 26(14): p. 1880-6). With the potential ability to normalize circadian disruption and the sleep/wake cycle in various mood disorders and sleep disturbances, small molecule inhibitors targeted towards the CSNK1D may possess therapeutic utility in a number of mood disorders including type 1 bipolar depression, type 2 bipolar depression, seasonal affective disorder, post-traumatic stress disorder, generalized anxiety disorder, dysthymia, obsessive compulsive disorder, schizophrenia, schizoaffective disorder, mixed episode bipolar disease, major depressive disorder, premenstrual dysphoric disorder, jet lag syndrome, familial advanced sleep phase syndrome, delayed sleep phase syndrome, non-24 hour sleep-wake phase disorder irregular sleep-wake rhythm disorder.

Casein kinases are a group of evolutionarily conserved serine/threonine kinases ubiquitously expressed in eukaryotes. This group includes two families: casein kinase 1 (CK1) and casein kinase 2 (CK2). Six different CK1 genes, CK1 α, γ1, γ2, γ3, δ, and ε have been identified in humans. Each isoform consists of a highly conserved kinase domain followed by a highly variable C-terminal non-catalytic domain. Members of the CK1 family are monomeric, constitutively active, co-factor independent kinases (Knippschild, U., et al., The casein kinase 1 family: participation in multiple cellular processes in eukaryotes. Cell Signal, 2005. 17(6): p. 675-89). By phosphorylating different substrates, such as cellular enzymes, transcriptional proteins, cytoskeletal and non-cytoskeletal proteins, viral oncogenes, and receptors, CK1 regulates diverse cellular processes, including cellular signaling, vesicular trafficking, cell division, and DNA repair pathways and circadian rhythms (Knippschild, U., et al., The casein kinase 1 family: participation in multiple cellular processes in eukaryotes. Cell Signal, 2005. 17(6): p. 675-89; Bischof, J., et al., CK1delta kinase activity is modulated by Chk1-mediated phosphorylation. PLoS One, 2013. 8(7): p. e68803; Schittek, B. and T. Sinnberg, Biological functions of casein kinase 1 isoforms and putative roles in tumorigenesis. Mol Cancer, 2014. 13: p. 231). Due to its role in tumor progression, small molecule inhibitors targeting CSNK1D may exhibit therapeutic utility in several cancers including gastroenteric, breast, renal, skin, hematological, colorectal, pancreatic, prostate, ovarian, bladder, liver, head/neck.

Genetic studies have shown an important role for casein kinase action on PER proteins in regulating circadian period. A mutation in the Syrian hamster CK1ε gene, tau, shortens the circadian period of behavioral rhythms. Biochemically, the tau mutation (CK1ε^{tan}, a T178C substitution) differentially affects the activity of the kinase protein, reducing general kinase activity while increasing activity at specific residues of the PER proteins (Gallego, M., et al., An opposite role for tau in circadian rhythms revealed by mathematical modeling. Proc Natl Acad Sci USA, 2006. 103(28): p. 10618-23; Lowrey, P. L., et al., Positional syntenic cloning and functional characterization of the mammalian circadian mutation tau. Science, 2000. 288(5465): p. 483-92). The tau mutation is a gain-of-function mutation with respect to circadian substrates, resulting in decreased PER stability and a reduction in circadian period length in tau mutant hamsters and mice (Gallego, M., et al., An opposite role for tau in circadian rhythms revealed by mathematical modeling. Proc Natl Acad Sci USA, 2006. 103(28): p. 10618-23; Meng, Q. J., et al., Setting clock speed in mammals: the CK1 epsilon tau mutation in mice accelerates circadian pacemakers by selectively destabilizing PERIOD proteins. Neuron, 2008. 58(1): p. 78-88). In humans, familial advanced sleep phase syndrome (FASPS) is a circadian-based sleep disorder, in which affected individuals have a short circadian period and an advanced phase of the sleep-wake cycle. One study identified a FASPS pedigree with a mutation in human PER2 (hPER2; S662G mutation); this mutation prevents a priming phosphorylation, thus preventing CK1-mediated phosphorylation (Toh, K. L., et al., An hPer2 phosphorylation site mutation infamilial advanced sleep phase syndrome. Science, 2001. 291(5506): p. 1040-3). A second study identified a dominant mutation within the kinase domain of CSNK1D in a family with FASPS (Xu, Y., et al., Functional consequences of a CK1delta mutation causing familial advanced sleep phase syndrome. Nature, 2005. 434(7033): p. 640-4). Modeling this mutation in mice also revealed alterations in period length.

CSNK1D has been linked to neurodegenerative disorders, including Alzheimer's disease (AD) Parkinson's disease (PD) and frontotemporal dementia (FTD). In particular, brain tissue from AD patients have been shown to express CSNK1D mRNA levels 30-fold higher than normal cells (Flajolet, M., et al., Regulation of Alzheimer's disease amyloid-beta formation by casein kinase I. Proc Natl Acad Sci USA, 2007. 104(10): p. 4159-64). β-Amyloid protein, present in a misfolded insoluble form in AD cells, has been shown to stimulate CSNK1D activity. Altogether these conditions promote an abnormal phosphorylation of tau protein, which is an AD-related substrate of the CSNK1D isoform. Recent reports have also highlighted a potential role of CSNK1D in neurological pathologies including Parkinson's and amyotrophic lateral sclerosis (Nonaka, T., et al., Phosphorylation of TAR DNA-binding Protein of 43 kDa (TDP-43) by Truncated Casein Kinase 1delta Triggers Mislocalization and Accumulation of TDP-43. J Biol Chem, 2016. 291(11): p. 5473-83; Morales-Garcia, J. A., et al., Biological and Pharmacological Characterization of Benzothiazole-Based CK-1delta Inhibitors in Models of Parkinson's Disease. ACS Omega, 2017. 2(8): p. 5215-5220). Since CSNK1D has been linked to both circadian disruption in neurodegenerative disorders and direct hyperphosphorylation of tau, α-synuclein and TDP-43, both disease modifying and symptomatic approaches can be explored for therapeutic utility in neurodegenerative disorders including those listed previously and also Down Syndrome, Progressive supranuclear palsy, Parkinsonism dementia complex of Guam, and Pick's Disease.

Small molecule inhibitors targeting CSNK1D may also attenuate addiction/substance abuse. Previous reports have implicated CSNK1D in addiction/substance abuse due to its phosphorylation/regulation of the protein cAMP-regulated neuronal phosphoprotein 32 (DARPP-32) (Nairn, A. C., et al., The role of DARPP-32 in the actions of drugs of abuse. Neuropharmacology, 2004; 47 (Suppl. 1), p. 14-23; Falcon, E., McClung, C. A., A role for the circadian genes in drug addiction. Neuropharmacology, 2009. 56 (Suppl. 1): p. 91-96). Additional reports have demonstrated that commercially available small molecule inhibitors PF-670462 of CSNK1D have shown efficacy in a number of addiction/substance abuse models including conditioned place preference with cocaine and alcohol reinstatement (Abaca C., Albrecht U., Spangel, R. Cocaine sensitization and reward are under the influence of circadian genes and rhythm. Proc. Natl. Acad. Sci. 2002. 99 (13), p. 9026-9030; Spangel, R., et al., The clock gene Per2 influences the glutamatergic system and modulates alcohol consumption. Nat. Med. 2005. 11 (1), p. 35-4; Perreu-Lenz, Vengeliene, V., et al., Inhibition of the casein kinase I epsilon/delta prevents relapse like alcohol drinking. Neuropsychopharmacology 2012, 37 (9) p. 2121-2131). Further published accounts have reported that PF-5006739 was also efficacious in attenuating fentanyl self-administration (Wager Travis T. et al., Casein Kinase 1δ/ε Inhibitor PF-5006739 Attenuates Opioid Drug-Seeking Behavior, 2014 Dec. 17; 5(12): p. 1253-65). Therefore, compounds that are synthesized to inhibit the activity of CSNK1D may exhibit therapeutic utility in a number of addictive/substance abuse indications involving chemicals (cocaine, opiate, tobacco, alcohol, amphetamines, inhalants, phencyclidine), impulse control disorders (intermittent explosive disorder, kleptomania, pyromania, gambling) and behavioral disturbances (food, sex, shopping, cutting, exercising, pain seeking).

Recently, published reports have also indicated a potential role for CSNK1D in the pathogenesis of various metabolic disorders. In the ob/ob and diet-induced obese mouse (two models of metabolic dysfunction), daily administration of the CSNK1D inhibitor PF-5006739 improve glucose tolerance (Cunningham, P. S., et al. Targeting the circadian clock via CK1d/e to improve glucose homeostasis in obesity. Sci Rep. 2016, 6, p. 29983). In addition, when a human adipocyte cell line was treated with CSNK1D specific inhibitors, increased basal and insulin-stimulated glucose uptake was measured (Xu, P., et al., Gene expression levels of Casein kinase 1 (CK1) isoforms are correlated to adiponectin levels in adipose tissue of morbid obese patients and site-specific phosphorylation mediated by CK1 influences multimerization of adiponectin. Mol Cell Endocrinol; 2015, 406: p. 87-101). Therefore, small molecule inhibitors may exert beneficial effects on glucose utilization in a number of metabolic diseases including Type 1 diabetes mellitus, idiopathic, type 2 diabetes mellitus, genetic defect of B-cell function, genetic defects of insulin action (type A insulin resistance, leprechaunism, Rabson-Mendahall syndrome, lipoatrophic diabetes), disease of exocrine pancreas (pancreatitis, neoplasia, trauma, cystic fibrosis, hemochromatosis, fibrocalculous pancreatopathy), endocrinopathies (Acromegaly, Cushing's syndrome, Glucagonoma, Pheochromocytoma, Hyperthyroidism, Somatostatinoma, Aldosteronoma), drug/chemical induced (Vacor, Pentamidine, Nicotinic acid, Glucocorticoids, Thyroid hormone, Diazoxide, 3-adrenergic agonists, Thiazides, Dilantin, ∝-Interferon), infections (congenital rubella, cytomegalovirus) uncommon forms (“stiff-man” syndrome, anti-insulin receptor antibodies), genetic syndromes (Down's syndrome, Klinefelter's syndrome, Turner's syndrome, Wolfram's syndrome, Friedreich's ataxia, Huntington's chorea, Laurence-Moon-Biedl syndrome, Myotonic dystrophy, Porphyria, Prader-Willi syndrome), gestational diabetes mellitus.

Small molecule inhibitors of CSNK1D have also been shown to be efficacious in a variety of pre-clinical pain models including von Frey to assess mechanical allodynia and also a model of inflammatory pain (Young, E. E., et al., Systems genetic and pharmacological analysis identifies candidate genes underlying mechanosensation in the von Frey test. Genes Brain Behav; 2016, 15(6): p. 604-615 and Kurihara, T., et al., Alleviation of behavioral hypersensitivity in mouse models of inflammatory pain with two structurally different casein kinase 1 (CK1) inhibitors. Mol Pain. 2014; 10: p. 17). Therefore, translational elements for developed small molecule inhibitors of CSNK1D may also be therapeutically beneficial in a number of pain indications including nociceptive (arthritis, mechanical back pain, post-surgical pain), inflammatory (gout, rheumatoid arthritis), neuropathic (neuropathy, radicular pain, trigeminal neuralgia), and functional (fibromyalgia, irritable bowel syndrome).

### SUMMARY OF THE INVENTION

Embodiments of the present invention relate to chemical entities, pharmaceutical compositions containing them, methods of making and purifying them, and methods for using them the treatment of diseases, disorders, and conditions associated with the CSNK1D modulation. An additional embodiment of the invention is a method of treating a subject suffering from or diagnosed with a disease, disorder, or condition associated with the CSNK1D modulation using at least one chemical entity of the invention.

Additional embodiments, features, and advantages of the invention will be apparent from the following detailed description and through practice of the invention.

Embodiments of this invention are compounds of Formula (I),

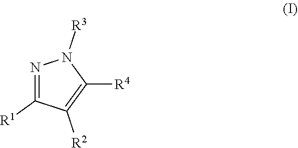

wherein
R¹ is selected from the group consisting of:
   (a) phenyl substituted with one or two halo members;
   (b) 5-fluoro-2-pyridyl optionally substituted with halo or C₁₋₃alkyl, 5-fluoro-3-pyridyl, 5-chloropyridin-2-yl, 3-chloropyridin-4-yl, 6-methoxypyridin-2-yl, 5-chloro-6-methylpyridin-2-yl, or 3,5-difluoropyridin-4-yl; and
   (c) oxazol-5-yl, thiazol-2-yl, thiazol-4-yl, or 1-methyl-1H-imidazol-4-yl; R² is selected from the group consisting of:
   (d) 4-pyridyl optionally substituted with one member selected from the group consisting of: halo, C₁₋₃haloalkyl, CH₂OH, OC₁₋₃alkyl, (C═O)—NHCH₃, NH₂, NH—(C═O)C₁₋₃alkyl, NH—(C═O)C₁₋₃haloalkyl, NH—(C═O)phenyl, NH—(C═O)cyclopropyl, and NH—(C═O)cyclopropyl wherein the cyclopropyl is substituted with one or two halo; 2,5-difluoro-4-pyridyl;

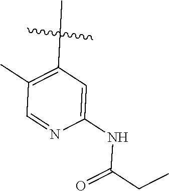 [SMILES: *c1cc(NC(=O)CC)ncc1C]

or 5-methylpyridin-2-amine;
(e) fused heteroaryl selected from the group consisting of: thieno[3,2-b]pyridinyl; pyrazolo[1,5-a]pyridin-5-yl optionally substituted with halo, C₁₋₃alkyl, or NH₂; 1H-pyrrolo[3,2-b]pyridinyl; 1H-pyrrolo[2,3-b]pyridinyl optionally substituted with one two or three members each independently selected from the group consisting of: halo, C₁₋₃alkyl, C₁₋₃haloalkyl, and CN; pyrazolo[1,5-a]pyrimidin-5-yl; [1,2,4]triazolo[1,5-a]pyridin-7-yl optionally substituted with C₁₋₃alkyl; 1H-pyrazolo[4,3-b]pyridin-7-yl optionally substituted with C₁₋₃alkyl; 1H-pyrazolo[3,4-b]pyridin-5-yl optionally substituted with C₁₋₃alkyl; 1H-pyrazolo[3,4-b]pyridinyl optionally substituted with one, two or three members each independently selected from the group consisting of: halo, C₁₋₃alkyl, C₁₋₃haloalkyl, and cyclopropyl; 2-methyl-2H-pyrazolo[4,3-b]pyridin-7-yl; 1-methyl-1H-pyrazolo[4,3-b]pyridin-7-yl; 2-methylpyrazolo[3,4-b]pyridin-4-yl; or 1H-pyrazolo[3,4-d]pyrimidin-4-yl optionally substituted with C₁₋₃alkyl; and
(f) 1,5-naphthyridin-4-yl optionally substituted with halo or OC₁₋₃alkyl;
R³ and R⁴ come together to form a group selected from the group consisting of:

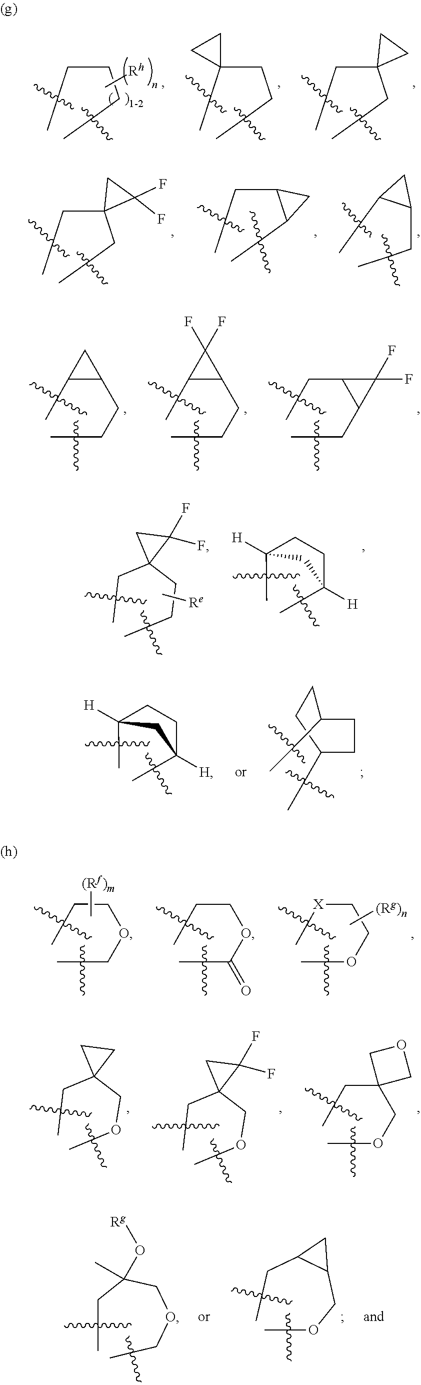

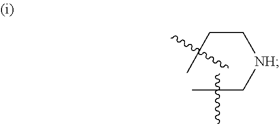 [SMILES: *CCNC*]

R^{f} is independently selected from the group consisting of: H, C₁₋₃alkyl, C₁₋₃haloalkyl, cyclopropyl, cyclobutyl, and two R^{f} members come together to form a C₃₋₆cycloalkyl wherein the C₃₋₆cycloalkyl is optionally substituted with one or two halo members;
R^{g} is H, halo, or C₁₋₃alkyl;
R^{h} is independently selected from the group consisting of H, halo, OH, C₁₋₃alkyl, CH₂OCH₃, CH₂CH₂OCH₃, C₁₋₃haloalkyl, CH₂OCHF₂, CH₂OCF₃, CN, and

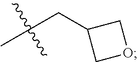 [SMILES: *CC1COC1]

X is selected from the group consisting of: a bond, CH₂, CH(CH₃), and CH₂CH₂;
m is 1, 2, 3 or 4; and
n is 1, 2, or 3;
   and pharmaceutically acceptable salts, isotopes, N-oxides, solvates, and stereoisomers of compounds of Formula (I).

### DETAILED DESCRIPTION OF THE INVENTION

As used herein, the terms “including”, “containing” and “comprising” are used in their open, non-limiting sense.

Unless qualified specifically in particular instances of use, the term “alkyl” refers to a straight- or branched-chain alkyl group having from 1 to 8 carbon atoms in the chain. Examples of alkyl groups include methyl (Me), ethyl (Et), n-propyl, isopropyl, butyl, isobutyl, sec-butyl, tert-butyl (tBu), pentyl, isopentyl, tert-pentyl, hexyl, isohexyl, and groups that in light of the ordinary skill in the art and the teachings provided herein would be considered equivalent to any one of the foregoing examples. “C₁₋₆alkyl” refers to straight- or branched-chain alkyl group having from 1 to 6 carbon atoms in the chain. “C₁₋₃alkyl” refers to straight- or branched-chain alkyl group having from 1 to 3 carbon atoms in the chain.

The term “cycloalkyl” refers to a saturated or partially saturated, monocyclic, fused polycyclic, or spiro polycyclic carbocycle having from 3 to 12 ring atoms per carbocycle. Illustrative examples of cycloalkyl groups include the following entities, in the form of properly bonded moieties:

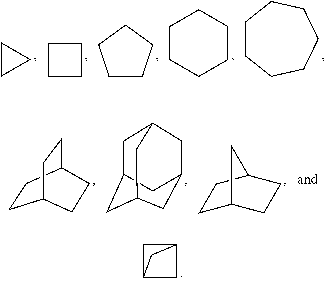

The term “halogen” or “halo” represents chlorine, fluorine, bromine, or iodine.

The term “haloalkyl” refers to a straight- or branched-chain alkyl group having from 1 to 6 carbon atoms in the chain optionally substituting hydrogens with halogens. The term “C₁₋₄ haloalkyl” as used here refers to a straight- or branched-chain alkyl group having from 1 to 4 carbon atoms in the chain, optionally substituting hydrogens with halogens. Examples of “haloalkyl” groups include trifluoromethyl (CF₃), difluoromethyl (CF₂H), monofluoromethyl (CH₂F), pentafluoroethyl (CF₂CF₃), tetrafluoroethyl (CHFCF₃), monofluoroethyl (CH₂CH₂F), trifluoroethyl (CH₂CF₃), tetrafluorotrifluoromethylethyl (CF(CF₃)₂), and groups that in light of the ordinary skill in the art and the teachings provided herein would be considered equivalent to any one of the foregoing examples.

The term “aryl” refers to a monocyclic, aromatic carbocycle (ring structure having ring atoms that are all carbon) having 6 atoms per ring (Carbon atoms in the aryl groups are sp2 hybridized.)

The term “phenyl” represents the following moiety:

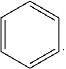

The term “heteroaryl” as used herein, refers to an aromatic monocyclic or multicyclic ring system comprising 5 to 14 ring atoms, wherein from 1 to 4 of the ring atoms is independently O, N or S and the remaining ring atoms are carbon atoms. In one embodiment, a heteroaryl group has 5 to 10 ring atoms. In another embodiment, a heteroaryl group is monocyclic and has 5 or 6 ring atoms. In another embodiment, a heteroaryl group is monocyclic and has 5 or 6 ring atoms and at least one nitrogen ring atom. A heteroaryl group is joined via a ring carbon atom and any nitrogen atom of a heteroaryl can be optionally oxidized to the corresponding N-oxide. The term “heteroaryl” also encompasses a heteroaryl group, as defined above, which has been fused to a benzene ring.

The term “5-membered heteroaryl” as used herein, refers to a heteroaryl group, as defined above, which has 5 ring atoms. Non-limiting examples of illustrative 5-membered heteroaryls

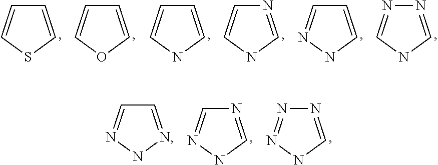

include:

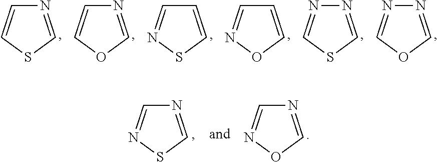

The term “6-membered heteroaryl” as used herein, refers to a heteroaryl group, as defined above, which has 6 ring atoms. Non-limiting examples of illustrative 6-membered heteroaryls include:

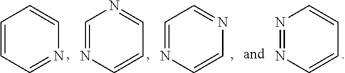

The term “5,6-fused bicyclic heteroaryl or 6,5-fused bicyclic heteroaryl” as used herein, refers to a heteroaryl group, as defined above, which has 9 ring atoms. Non-limiting examples of illustrative 5,6-fused bicyclic heteroaryl or 6,5-fused bicyclic heteroaryl include:

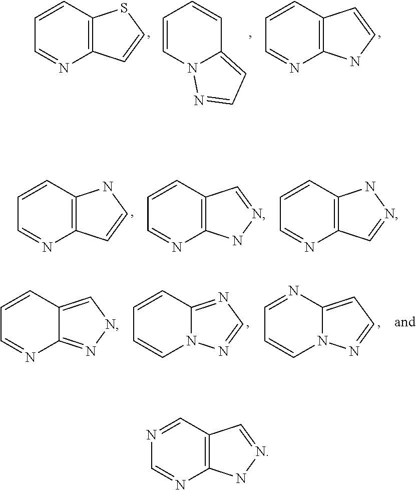

The term “6,6-fused bicyclic heteroaryl” as used herein, refers to a heteroaryl group, as defined above, which has 9 ring atoms. Non-limiting examples of illustrative 6,6-fused bicyclic heteroaryl include:

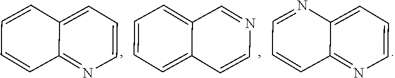

The term “heterocycloalkyl” as used herein, refers to a ring system which is non-aromatic, 1 to 4 of the ring atoms is independently O, N or S and the remaining ring atoms are carbon atoms, which may optionally be fused to another ring (aromatic or heteroaromatic). Non-limiting examples of illustrative heterocycloalkyl include:

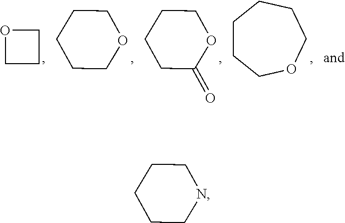

Those skilled in the art will recognize that the species of heteroaryl, heterocycloalkyl, cycloalkyl, and aryl groups listed or illustrated above are not exhaustive, and that additional species within the scope of these defined terms may also be selected.

The term “substituted” means that the specified group or moiety bears one or more substituents. The term “unsubstituted” means that the specified group bears no substituents. The term “optionally substituted” means that the specified group is unsubstituted or substituted by one or more substituents. Where the term “substituted” is used to describe a structural system, the substitution is meant to occur at any valency-allowed position on the system.

The term “variable point of attachment” means that a group is allowed to be attached at more than one alternative position in a structure. The attachment will always replace a hydrogen atom on one of the ring atoms. In other words, all permutations of bonding are represented by the single diagram, as shown in the illustrations below

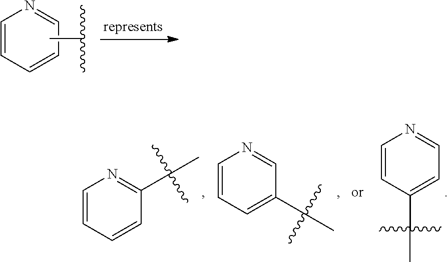

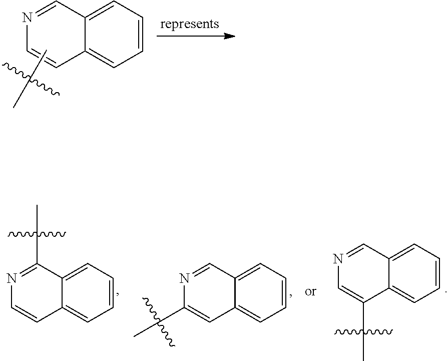

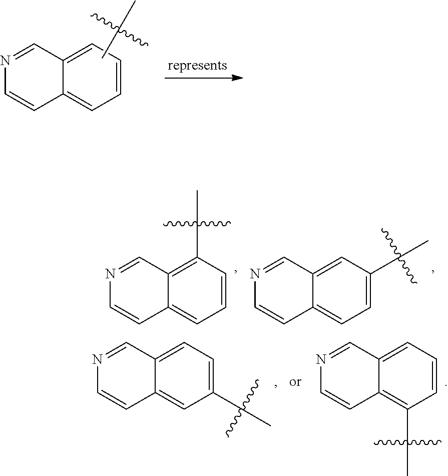

Those skilled in the art will recognize that that if more than one such substituent is present for a given ring, the bonding of each substituent is independent of all of the others. The groups listed or illustrated above are not exhaustive.

The term “substituted” means that the specified group or moiety bears one or more substituents. The term “unsubstituted” means that the specified group bears no substituents. The term “optionally substituted” means that the specified group is unsubstituted or substituted by one or more substituents. Where the term “substituted” is used to describe a structural system, the substitution is meant to occur at any valency-allowed position on the system.

Any formula given herein is intended to represent compounds having structures depicted by the structural formula as well as certain variations or forms. In particular, compounds of any formula given herein may have asymmetric centers and therefore exist in different enantiomeric forms. All optical isomers and stereoisomers of the compounds of the general formula, and mixtures thereof, are considered within the scope of such formula.

The compounds of this invention may possess one or more asymmetric centers; such compounds can therefore be produced as individual (R)- or (S)-stereoisomers or as mixtures thereof. Thus, any formula given herein is intended to represent a racemate, one or more of its enantiomeric forms, one or more of its diastereomeric forms, and mixtures thereof. Additionally, any formula given herein is intended to refer also to any one of: hydrates, solvates, polymorphs and of such compounds, and mixtures thereof, even if such forms are not listed explicitly.

The absolute stereochemistry is specified according to the Cahn-Ingold-Prelog R-S system. Chiral centers, of which the absolute configurations are known, are labelled by prefixes R and S, assigned by the standard sequence-rule procedure, and preceded when necessary by the appropriate locants (Pure & Appl. Chem. 45, 1976, 11-30).

The term “R” at a stereocenter designates that the stereocenter is purely of the R-configuration as defined in the art; likewise, the term “S” means that the stereocenter is purely of the S-configuration. As used herein, the term “RS” refers to a stereocenter that exists as a mixture of the R- and S-configurations.

Compounds containing one stereocenter drawn without a stereo bond designation are a mixture of 2 enantiomers. Compounds containing 2 stereocenters both drawn without stereo bond designations are a mixture of 4 diastereomers. Compounds with 2 stereocenters both labeled “RS” and drawn with stereo bond designations are a 2-component mixture with relative stereochemistry as drawn. Unlabeled stereocenters drawn without stereo bond designations are a mixture of the R- and S-configurations. For unlabeled stereocenters drawn with stereo bond designations, the absolute stereochemistry is as depicted.

Certain examples contain chemical structures that are depicted or labelled as an (*R) or (*S). When (*R) or (*S) is used in the name of a compound or in the chemical representation of the compound, it is intended to convey that the compound is a pure single isomer at that stereocenter; however, absolute configuration of that stereocenter has not been established. Thus, a compound designated as (*R) refers to a compound that is a pure single isomer at that stereocenter with an absolute configuration of either (R) or (S), and a compound designated as (*S) refers to a compound that is a pure single isomer at that stereocenter with an absolute configuration of either (R) or (S). For example, (*R)-4-[2-(4-Fluorophenyl)-7-methyl-4,5,6,7-tetrahydropyrazolo[1,5-a]pyridin-3-yl]-1H-pyrazolo[3,4-b]pyridine:

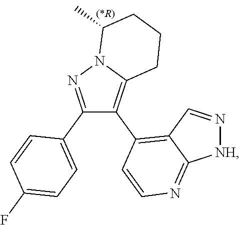 [SMILES: C[C@@H]1CCCc2c(-c3ccnc4[nH]ncc34)c(-c3ccc(F)cc3)nn21]

refers to a compound that is either:

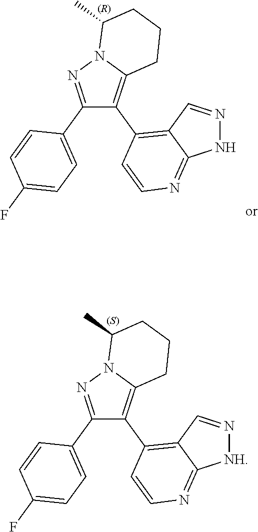

Pseudoasymmetric stereogenic centers are treated in the same way as chiral centers, but are given lower-case symbols, r or s (Angew. Chem. Int. Ed. Engl. 1982, 21, 567-583).

Reference to a compound herein stands for a reference to any one of: (a) the actually recited form of such compound, and (b) any of the forms of such compound in the medium in which the compound is being considered when named. For example, reference herein to a compound such as R—COOH, encompasses reference to any one of: for example, R—COOH(s), R—COOH(sol), and R—COO-(sol). In this example, R—COOH(s) refers to the solid compound, as it could be for example in a tablet or some other solid pharmaceutical composition or preparation; R—COOH(sol) refers to the undissociated form of the compound in a solvent; and R—COO-(sol) refers to the dissociated form of the compound in a solvent, such as the dissociated form of the compound in an aqueous environment, whether such dissociated form derives from R—COOH, from a salt thereof, or from any other entity that yields R—COO— upon dissociation in the medium being considered. In another example, an expression such as “exposing an entity to compound of formula R—COOH” refers to the exposure of such entity to the form, or forms, of the compound R—COOH that exists, or exist, in the medium in which such exposure takes place. In still another example, an expression such as “reacting an entity with a compound of formula R—COOH” refers to the reacting of (a) such entity in the chemically relevant form, or forms, of such entity that exists, or exist, in the medium in which such reacting takes place, with (b) the chemically relevant form, or forms, of the compound R—COOH that exists, or exist, in the medium in which such reacting takes place. In this regard, if such entity is for example in an aqueous environment, it is understood that the compound R—COOH is in such same medium, and therefore the entity is being exposed to species such as R—COOH(aq) and/or R—COO-(aq), where the subscript “(aq)” stands for “aqueous” according to its conventional meaning in chemistry and biochemistry. A carboxylic acid functional group has been chosen in these nomenclature examples; this choice is not intended, however, as a limitation but it is merely an illustration. It is understood that analogous examples can be provided in terms of other functional groups, including but not limited to hydroxyl, basic nitrogen members, such as those in amines, and any other group that interacts or transforms according to known manners in the medium that contains the compound. Such interactions and transformations include, but are not limited to, dissociation, association, tautomerism, solvolysis, including hydrolysis, solvation, including hydration, protonation, and deprotonation. No further examples in this regard are provided herein because these interactions and transformations in a given medium are known by any one of ordinary skill in the art.

Any formula given herein is also intended to represent unlabeled forms as well as isotopically labeled forms of the compounds. Isotopically labeled compounds have structures depicted by the formulas given herein except that one or more atoms are replaced by an atom having a selected atomic mass or mass number in an enriched form. Examples of isotopes that can be incorporated into compounds of the invention in a form that exceeds natural abundances include isotopes of hydrogen, carbon, nitrogen, oxygen, phosphorous, fluorine, chlorine, and iodine, such as ²H (or chemical symbol D), ³H (or chemical symbol T), ¹¹C, ¹³C, ¹⁴C, ¹⁵N, ¹⁸O, ¹⁷O ³¹P, ³²P, ³⁵S, ¹⁸F, ³⁶Cl, and ¹²⁵I, respectively. Such isotopically labelled compounds are useful in metabolic studies (preferably with ¹⁴C), reaction kinetic studies (with, for example ²H or ³H), detection or imaging techniques [such as positron emission tomography (PET) or single-photon emission computed tomography (SPECT)] including drug or substrate tissue distribution assays, or in radioactive treatment of patients. In particular, an ¹⁸F or ¹¹C labeled compound may be particularly preferred for PET or SPECT studies. Further, substitution with heavier isotopes such as deuterium (i.e., ²H, or D) may afford certain therapeutic advantages resulting from greater metabolic stability, for example increased in vivo half-life or reduced dosage requirements. Isotopically labeled compounds of this invention can generally be prepared by carrying out the procedures disclosed in the schemes or in the examples and preparations described below by substituting a readily available isotopically labeled reagent for a non-isotopically labeled reagent.

When referring to any formula given herein, the selection of a particular moiety from a list of possible species for a specified variable is not intended to define the same choice of the species for such variable appearing elsewhere. In other words, where a variable appears more than once, the choice of the species from a specified list is independent of the choice of the species for the same variable elsewhere in the formula, unless stated otherwise.

The term Cₙ₋ₘ alkyl refers to an aliphatic chain, whether straight or branched, with a total number N of carbon members in the chain that satisfies n≤N≤m, with m>n.

When the same plurality of substituents is assigned to various groups, the specific individual substituent assignment to each of such groups is meant to be independently made with respect to the specific individual substituent assignments to the remaining groups. By way of illustration, but not as a limitation, if each of the groups Q and R can be H or F, the choice of H or F for Q is made independently of the choice of H or F for R, so the choice of assignment for Q does not determine or condition the choice of assignment for R, or vice-versa, unless it is expressly indicated otherwise. Illustrative claim recitation in this regard would read as “each of Q and R is independently H or F”, or “each of Q and R is independently selected from the group consisting of H and F”.

Unless indicated otherwise, the description or naming of a particular compound in the specification and claims is intended to include both individual enantiomers and mixtures, racemic or otherwise, thereof. The methods for the determination of stereochemistry and the separation of stereoisomers are well-known in the art.

In another example, a zwitterionic compound would be encompassed herein by referring to a compound that is known to form a zwitterion, even if it is not explicitly named in its zwitterionic form. Terms such as zwitterion, zwitterions, and their synonyms zwitterionic compound(s) are standard IUPAC-endorsed names that are well known and part of standard sets of defined scientific names. In this regard, the name zwitterion is assigned the name identification CHEBI.27369 by the Chemical Entities of Biological Interest (ChEBI) dictionary of molecular entities. As generally well known, a zwitterion or zwitterionic compound is a neutral compound that has formal unit charges of opposite sign. Sometimes these compounds are referred to by the term “inner salts”. Other sources refer to these compounds as “dipolar ions”, although the latter term is regarded by still other sources as a misnomer. As a specific example, aminoethanoic acid (the amino acid glycine) has the formula H₂NCH₂COOH, and it exists in some media (in this case in neutral media) in the form of the zwitterion ⁺H₃NCH₂COO. Zwitterions, zwitterionic compounds, inner salts and dipolar ions in the known and well-established meanings of these terms are within the scope of this invention, as would in any case be so appreciated by those of ordinary skill in the art. Because there is no need to name each and every embodiment that would be recognized by those of ordinary skill in the art, no structures of the zwitterionic compounds that are associated with the compounds of this invention are given explicitly herein. They are, however, part of the embodiments of this invention. No further examples in this regard are provided herein because the interactions and transformations in a given medium that lead to the various forms of a given compound are known by any one of ordinary skill in the art.

When referring to any formula given herein, the selection of a particular moiety from a list of possible species for a specified variable is not intended to define the same choice of the species for the variable appearing elsewhere. In other words, where a variable appears more than once, the choice of the species from a specified list is independent of the choice of the species for the same variable elsewhere in the formula, unless stated otherwise.

By way of a first example on substituent terminology, if substituent S¹ₑₓₐₘₚₗₑ is one of S₁ and S₂, and substituent S²ₑₓₐₘₚₗₑ is one of S₃ and S₄, then these assignments refer to embodiments of this invention given according to the choices S¹ₑₓₐₘₚₗₑ is S₁ and S²ₑₓₐₘₚₗₑ is S₃; S¹ₑₓₐₘₚₗₑ is S₁ and S²ₑₓₐₘₚₗₑ is S₄; S¹ₑₓₐₘₚₗₑ is S₂ and S²ₑₓₐₘₚₗₑ is S₃; S¹ₑₓₐₘₚₗₑ is S₂ and S²ₑₓₐₘₚₗₑ is S₄; and equivalents of each one of such choices. The shorter terminology “S¹ₑₓₐₘₚₗₑ is one of S₁ and S₂, and S²ₑₓₐₘₚₗₑ is one of S₃ and S₄” is accordingly used herein for the sake of brevity, but not by way of limitation. The foregoing first example on substituent terminology, which is stated in generic terms, is meant to illustrate the various substituent assignments described herein.

Furthermore, when more than one assignment is given for any member or substituent, embodiments of this invention comprise the various groupings that can be made from the listed assignments, taken independently, and equivalents thereof. By way of a second example on substituent terminology, if it is herein described that substituent Sₑₓₐₘₚₗₑ is one of S₁, S₂, and S₃, this listing refers to embodiments of this invention for which Sₑₓₐₘₚₗₑ is S₁; Sₑₓₐₘₚₗₑ is S₂; Sₑₓₐₘₚₗₑ is S₃; Sₑₓₐₘₚₗₑ is one of S₁ and S₂; Sₑₓₐₘₚₗₑ is one of S₁ and S₃; Sₑₓₐₘₚₗₑ is one of S₂ and S₃; Sₑₓₐₘₚₗₑ is one of S₁, S₂ and S₃; and Sₑₓₐₘₚₗₑ is any equivalent of each one of these choices. The shorter terminology “Sₑₓₐₘₚₗₑ is one of S₁, S₂, and S₃” is accordingly used herein for the sake of brevity, but not by way of limitation. The foregoing second example on substituent terminology, which is stated in generic terms, is meant to illustrate the various substituent assignments described herein.

The nomenclature “Cᵢ-Cⱼ” or “Cᵢ₋ⱼ” with j>i, when applied herein to a class of substituents, is meant to refer to embodiments of this invention for which each and every one of the number of carbon members, from i to j including i and j, is independently realized. By way of example, the term C₁-C₃ refers independently to embodiments that have one carbon member (C₁), embodiments that have two carbon members (C₂), and embodiments that have three carbon members (C₃).

A “pharmaceutically acceptable salt” is intended to mean a salt of an acid or base of a compound represented by Formula (I) that is non-toxic, biologically tolerable, or otherwise biologically suitable for administration to the subject. See, generally, S. M. Berge, et al., “Pharmaceutical Salts”, J. Pharm. Sci., 1977, 66:1-19, and Handbook of Pharmaceutical Salts, Properties, Selection, and Use, Stahl and Wermuth, Eds., Wiley-VCH and VHCA, Zurich, 2002. Preferred pharmaceutically acceptable salts are those that are pharmacologically effective and suitable for contact with the tissues of patients without undue toxicity, irritation, or allergic response.

A compound of Formula (I) may possess a sufficiently acidic group, a sufficiently basic group, or both types of functional groups, and accordingly react with a number of inorganic or organic bases, and inorganic and organic acids, to form a pharmaceutically acceptable salt.

Examples of pharmaceutically acceptable salts include sulfates, pyrosulfates, bisulfates, sulfites, bisulfites, phosphates, monohydrogen-phosphates, dihydrogenphosphates, metaphosphates, pyrophosphates, chlorides, bromides, iodides, acetates, propionates, decanoates, caprylates, acrylates, formates, isobutyrates, caproates, heptanoates, propiolates, oxalates, malonates, succinates, suberates, sebacates, fumarates, maleates, butyne-1,4-dioates, hexyne-1,6-dioates, benzoates, chlorobenzoates, methylbenzoates, dinitrobenzoates, hydroxybenzoates, methoxybenzoates, phthalates, sulfonates, xylenesulfonates, phenylacetates, phenylpropionates, phenylbutyrates, citrates, lactates, γ-hydroxybutyrates, glycolates, tartrates, methane-sulfonates, propanesulfonates, naphthalene-1-sulfonates, naphthalene-2-sulfonates, and mandelates.

Compounds of Formula (I) may contain at least one nitrogen of basic character, so desired pharmaceutically acceptable salts may be prepared by any suitable method available in the art, for example, treatment of the free base with an inorganic acid, such as hydrochloric acid, hydrobromic acid, sulfuric acid, sulfamic acid, nitric acid, boric acid, phosphoric acid, and the like, or with an organic acid, such as acetic acid, phenylacetic acid, propionic acid, stearic acid, lactic acid, ascorbic acid, maleic acid, hydroxymaleic acid, isethionic acid, succinic acid, valeric acid, fumaric acid, malonic acid, pyruvic acid, oxalic acid, glycolic acid, salicylic acid, oleic acid, palmitic acid, lauric acid, a pyranosidyl acid, such as glucuronic acid or galacturonic acid, an alpha-hydroxy acid, such as mandelic acid, citric acid, or tartaric acid, an amino acid, such as aspartic acid or glutamic acid, an aromatic acid, such as benzoic acid, 2-acetoxybenzoic acid, naphthoic acid, or cinnamic acid, a sulfonic acid, such as laurylsulfonic acid, p-toluenesulfonic acid, methanesulfonic acid, ethanesulfonic acid, any compatible mixture of acids such as those given as examples herein, and any other acid and mixture thereof that are regarded as equivalents.

Compounds of Formula (I) may contain a carboxylic acid moiety, a desired pharmaceutically acceptable salt may be prepared by any suitable method, for example, treatment of the free acid with an inorganic or organic base, such as an amine (primary, secondary or tertiary), an alkali metal hydroxide, alkaline earth metal hydroxide, any compatible mixture of bases such as those given as examples herein, and any other base and mixture thereof that are regarded as equivalents or acceptable substitutes in light of the ordinary level of skill in this technology. Illustrative examples of suitable salts include organic salts derived from amino acids, such as glycine and arginine, ammonia, carbonates, bicarbonates, primary, secondary, and tertiary amines, and cyclic amines, such as benzylamines, pyrrolidines, piperidine, morpholine, piperazine, N-methyl-glucamine and tromethamine and inorganic salts derived from sodium, calcium, potassium, magnesium, manganese, iron, copper, zinc, aluminum, and lithium.

The compounds of the invention, including their pharmaceutically acceptable salts, whether alone or in combination, (collectively, “active agent” or “active agents”) of the present invention are useful as CSNK1D-modulators in the methods of the invention. Such methods for modulating CSNK1D comprise the use of a therapeutically effective amount of at least one chemical entity of the invention.

In some embodiments, the CSNK1D modulator is an inhibitor and is used in a subject diagnosed with or suffering from a disease, disorder, or condition associated with protein kinase CSNK1D activity, such as those described herein. Symptoms or disease states are intended to be included within the scope of “disease, disorders or conditions.”

Accordingly, the invention relates to methods of using the active agents described herein to treat subjects diagnosed with or suffering from a disease, disorder, or condition associated with the protein kinase CSNK1D activity. The term “treat” or “treating” as used herein is intended to refer to administration of an active agent or composition of the invention to a subject for the purpose of effecting a therapeutic or prophylactic benefit through modulation of protein kinase CSNK1D activity. Treating includes reversing, ameliorating, alleviating, inhibiting the progress of, lessening the severity of, or preventing a disease, disorder, or condition, or one or more symptoms of such disease, disorder or condition associated with the CSNK1D modulation. The term “subject” refers to a mammalian patient in need of such treatment, such as a human.

The term “composition” refers to a product that includes the specified ingredients in therapeutically effective amounts, as well as any product that results, directly, or indirectly, from combinations of the specified ingredients in the specified amounts.

The term “CSNK1D inhibitor” is intended to encompass a compound that interacts with protein kinase CSNK1D to substantially reduce or eliminate its catalytic activity, thereby increasing the concentrations of its substrate(s). The term “CSNK1D-modulated” is used to refer to the condition of being affected by the modulation of the activity of protein kinase CSNK1D including the condition of being affected by the inhibition of the CSNK1D activity. The disclosure is directed to methods for treating, ameliorating and/or preventing neurodegenerative diseases and/or disorders, psychiatric disorders, and cancers by the administration of therapeutically effective amounts of protein kinase CSNK1D modulators to subjects in need thereof.

The term “modulators” include both inhibitors and activators, where “inhibitors” refer to compounds that decrease, prevent, inactivate, desensitize, or down-regulate the CSNK1D expression or activity, and “activators” are compounds that increase, activate, facilitate, sensitize, or up-regulate CSNK1D expression or activity.

As used herein, unless otherwise noted, the term “affect” or “affected” (when referring to a disease, condition or disorder that is affected by inhibition of CSNK1D) includes a reduction in the frequency and/or severity of one or more symptoms or manifestations of said disease, condition or disorder; and/or include the prevention of the development of one or more symptoms or manifestations of said disease, condition or disorder or the development of the disease, condition or disorder.

In treatment methods according to the invention, a therapeutically effective amount of at least one active agent according to the invention is administered to a subject suffering from or diagnosed as having such a disease, disorder, or condition. A “therapeutically effective amount” means an amount or dose sufficient to generally bring about the desired therapeutic or prophylactic benefit in subjects in need of such treatment for the designated disease, disorder, or condition. Effective amounts or doses of the active agents of the present invention may be ascertained by routine methods such as modeling, dose escalation studies or clinical trials, and by taking into consideration routine factors, e.g., the mode or route of administration or drug delivery, the pharmacokinetics of the agent, the severity and course of the disease, disorder, or condition, the subject's previous or ongoing therapy, the subject's health status and response to drugs, and the judgment of the treating physician. For a 70-kg human, an illustrative range for a suitable dosage amount is from about 1 to 1000 mg/day in single or multiple dosage units (e.g., BID, TID, QID or as required by modality). For example, a suitable dosage amount is from about 100 to 300 mg/day in single or multiple dosage units.

Once improvement of the subject's disease, disorder, or condition has occurred, the dose may be adjusted for preventive or maintenance treatment. For example, the dosage or the frequency of administration, or both, may be reduced as a function of the symptoms, to a level at which the desired therapeutic or prophylactic effect is maintained. Of course, if symptoms have been alleviated to an appropriate level, treatment may cease. Subjects may, however, require intermittent treatment on a long-term basis upon any recurrence of symptoms.

In addition, the compounds of the invention are envisaged for use alone, in combination with one or more of other compounds of this invention, or in combination with additional active ingredients in the treatment of the conditions discussed below. The additional active ingredients may be co-administered separately with at least one compound of the invention, with active agents of the invention or included with such an agent in a pharmaceutical composition according to the invention. In an illustrative embodiment, additional active ingredients are those that are known or discovered to be effective in the treatment of conditions, disorders, or diseases associated with protein kinase CSNK1D modulation, such as another protein kinase CSNK1D inhibitor or a compound active against another target associated with the particular condition, disorder, or disease. The combination may serve to increase efficacy (e.g., by including in the combination a compound potentiating the potency or effectiveness of an agent according to the invention), decrease one or more side effects, or decrease the required dose of the active agent according to the invention.

When referring to inhibiting the target, an “effective amount” means an amount sufficient to affect protein kinase CSNK1D modulation.

The active agents of the invention are envisaged for use, alone or in combination with one or more additional active ingredients, to formulate pharmaceutical compositions of the invention. A pharmaceutical composition of the invention comprises a therapeutically effective amount of at least one active agent in accordance with the invention.

Pharmaceutically acceptable excipients commonly used in pharmaceutical compositions are substances that are non-toxic, biologically tolerable, and otherwise biologically suitable for administration to a subject, such as an inert substance, added to a pharmacological composition or otherwise used as a vehicle, carrier, or diluent to facilitate administration of an agent and that is compatible therewith. Examples of such excipients include calcium carbonate, calcium phosphate, various sugars and types of starch, cellulose derivatives, gelatin, vegetable oils, and polyethylene glycols.

Delivery forms of the pharmaceutical compositions containing one or more dosage units of the active agents may be prepared using pharmaceutically acceptable excipients and compounding techniques known or that become available to those of ordinary skill in the art. The compositions may be administered in the inventive methods by a suitable route of delivery, e.g., oral, parenteral, rectal, topical, or ocular routes, or by inhalation.

The preparation may be in the form of tablets, capsules, sachets, dragees, powders, granules, lozenges, powders for reconstitution, liquid preparations, or suppositories. The compositions may be formulated for any one of a plurality of administration routes, such as intravenous infusion, topical administration, or oral administration. Preferably, the compositions may be formulated for oral administration.

For oral administration, the active agents of the invention can be provided in the form of tablets or capsules, or as a solution, emulsion, or suspension. To prepare the oral compositions, the active agents may be formulated to yield a dosage of, e.g., for a 70-kg human, an illustrative range for a suitable dosage amount is from about 1 to 1000 mg/day in single or multiple dosage units. Preferably, a suitable dosage amount is from about 100 to 300 mg/day in single or multiple dosage units.

Oral tablets may include the active ingredient(s) mixed with compatible pharmaceutically acceptable excipients such as diluents, disintegrating agents, binding agents, lubricating agents, sweetening agents, flavoring agents, coloring agents and preservative agents. Suitable inert fillers include sodium and calcium carbonate, sodium and calcium phosphate, lactose, starch, sugar, glucose, methyl cellulose, magnesium stearate, mannitol, sorbitol, and the like. Exemplary liquid oral excipients include ethanol, glycerol, water, and the like. Starch, polyvinyl-pyrrolidone (PVP), sodium starch glycolate, microcrystalline cellulose, and alginic acid are exemplary disintegrating agents. Binding agents may include starch and gelatin. The lubricating agent, if present, may be magnesium stearate, stearic acid or talc. If desired, the tablets may be coated with a material such as glyceryl monostearate or glyceryl distearate to delay absorption in the gastrointestinal tract or may be coated with an enteric coating.

Capsules for oral administration include hard and soft gelatin or (hydroxypropyl)methyl cellulose capsules. To prepare hard gelatin capsules, active ingredient(s) may be mixed with a solid, semi-solid, or liquid diluent. Liquids for oral administration may be in the form of suspensions, solutions, emulsions, or syrups or may be lyophilized or presented as a dry product for reconstitution with water or other suitable vehicle before use. Such liquid compositions may optionally contain: pharmaceutically-acceptable excipients such as suspending agents (for example, sorbitol, methyl cellulose, sodium alginate, gelatin, hydroxyethylcellulose, carboxymethylcellulose, aluminum stearate gel and the like); non-aqueous vehicles, e.g., oil (for example, almond oil or fractionated coconut oil), propylene glycol, ethyl alcohol, or water; preservatives (for example, methyl or propyl p-hydroxybenzoate or sorbic acid); wetting agents such as lecithin; and, if desired, flavoring or coloring agents.

The active agents of this invention may also be administered by non-oral routes. For example, compositions may be formulated for rectal administration as a suppository, enema or foam. For parenteral use, including intravenous, intramuscular, intraperitoneal, or subcutaneous routes, the agents of the invention may be provided in sterile aqueous solutions or suspensions, buffered to an appropriate pH and isotonicity or in parenterally acceptable oil. Suitable aqueous vehicles include Ringer's solution and isotonic sodium chloride. Such forms may be presented in unit-dose form such as ampules or disposable injection devices, in multi-dose forms such as vials from which the appropriate dose may be withdrawn, or in a solid form or pre-concentrate that can be used to prepare an injectable formulation. Illustrative infusion doses range from about 1 to 1000 μg/kg/minute of agent admixed with a pharmaceutical carrier over a period ranging from several minutes to several days.

For topical administration, the agents may be mixed with a pharmaceutical carrier at a concentration of about 0.01% to about 20% of drug to vehicle, preferably 0.1% to 10%. Another mode of administering the agents of the invention may utilize a patch formulation to affect transdermal delivery.

Active agents may alternatively be administered in methods of this invention by inhalation, via the nasal or oral routes, e.g., in a spray formulation also containing a suitable carrier.

In a further embodiment, the invention is directed to a method of treating a subject suffering from or diagnosed with a disease, disorder, or condition associated with CSNK1D modulation, comprising administering to the subject in need of such treatment a therapeutically effective amount of the active agent.

The compounds of Formula (I) are useful in methods for treating, ameliorating and/or preventing a disease, a condition or a disorder that is affected by the inhibition of CSNK1D. Such methods comprise administering to a subject, including an animal, a mammal, and a human in need of such treatment, amelioration and/or prevention, a therapeutically effective amount of a compound of Formula (I), or an enantiomer, diastereomer, solvate or pharmaceutically acceptable salt thereof.

In particular, the compounds of Formula (I), or pharmaceutically acceptable salts, isotopes, N-oxides, solvates and stereoisomers thereof, are useful for treating, ameliorating and/or preventing neurodegenerative diseases and/or disorders, psychiatric disorders, and cancers. More particularly, the compounds of Formula (I), or pharmaceutically acceptable salts, isotopes, N-oxides, solvates and stereoisomers thereof, are useful for treating, ameliorating and/or preventing mood or psychiatric disorders, neurodegenerative diseases, oncology indications, addiction or substance abuse indications, metabolic indications and pain by administering to a subject in need thereof a therapeutically effective amount of a compound of Formula (I), or a pharmaceutically acceptable salt, isotope, N-oxide, solvate or stereoisomer thereof as herein defined.

Mood/psychiatric disorders include: type 1 bipolar depression, type 2 bipolar depression, seasonal affective disorder, post-traumatic stress disorder, generalized anxiety disorder, dysthymia, obsessive compulsive disorder, schizophrenia, schizoaffective disorder, mixed episode bipolar disease, major depressive disorder, premenstrual dysphoric disorder, jet lag syndrome, familial advanced sleep phase syndrome, delayed sleep phase syndrome, non-24 hour sleep-wake phase disorder and irregular sleep-wake rhythm disorder.

Neurodegenerative diseases include Alzheimer's disease, Parkinson's disease, Amyotrophic lateral sclerosis, Frontotemporal dementia, Down Syndrome, Progressive supranuclear palsy, parkinsonism dementia complex of Guam, and Pick's disease.

Oncology indications include: gastroenteric, breast, renal, skin, hematological, colorectal, pancreatic, prostate, ovarian, bladder, liver, and head/neck.

Addiction and substance abuse indications involving chemicals (such as cocaine, opiate, tobacco, alcohol, amphetamines, inhalants, and phencyclidine), impulse control disorders (such as intermittent explosive disorder, kleptomania, pyromania, and gambling), and behavioral disturbances (such as food, sex, shopping, cutting, exercising, and pain seeking).

Metabolic diseases include: type 1 diabetes mellitus, idiopathic, type 2 diabetes mellitus, genetic defect of B-cell function, genetic defects of insulin action (such as type A insulin resistance, leprechaunism, Rabson-Mendahall syndrome, and lipoatrophic diabetes), disease of exocrine pancreas (such as pancreatitis, neoplasia, trauma, cystic fibrosis, hemochromatosis, and fibrocalculous pancreatopathy), endocrinopathies (such as Acromegaly, Cushing's syndrome, Glucagonoma, Pheochromocytoma, Hyperthyroidism, Somatostatinoma, and Aldosteronoma), drug/chemical induced (such as Vacor, Pentamidine, Nicotinic acid, Glucocorticoids, Thyroid hormone, Diazoxide, ß-adrenergic agonists, Thiazides, Dilantin, and ∝-Interferon), infections (such as congenital rubella, and cytomegalovirus) uncommon forms (such as “stiff-man” syndrome and anti-insulin receptor antibodies), genetic syndromes (such as Down's syndrome, Klinefelter's syndrome, Turner's syndrome, Wolfram's syndrome, Friedreich's ataxia, Huntington's chorea, Laurence-Moon-Biedl syndrome, Myotonic dystrophy, Porphyria, and Prader-Willi syndrome), and gestational diabetes mellitus.

Pain includes nociceptive (such as arthritis, mechanical back pain, and post-surgical pain), inflammatory (such as gout and rheumatoid arthritis), neuropathic (such as neuropathy, radicular pain, and trigeminal neuralgia), and functional (such as fibromyalgia and irritable bowel syndrome).

Other embodiments of this invention provide for a method for modulating protein kinase CSNK1D activity, including when such receptor is in a subject, comprising exposing protein kinase CSNK1D to a therapeutically effective amount of at least one compound selected from compounds of the invention.

Embodiments of this invention are compounds of Formula (I),

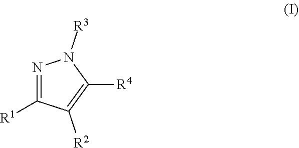

wherein
R¹ is selected from the group consisting of:
   (a) phenyl substituted with one or two halo members;
   (b) 5-fluoro-2-pyridyl optionally substituted with halo or C₁₋₃alkyl, 5-fluoro-3-pyridyl, 5-chloropyridin-2-yl, 3-chloropyridin-4-yl, 6-methoxypyridin-2-yl, 5-chloro-6-methylpyridin-2-yl, or 3,5-difluoropyridin-4-yl; and
   (c) oxazol-5-yl, thiazol-2-yl, thiazol-4-yl, or 1-methyl-1H-imidazol-4-yl;
R² is selected from the group consisting of:
   (d) 4-pyridyl optionally substituted with one member selected from the group consisting of: halo, C₁₋₃haloalkyl, CH₂OH, OC₁₋₃alkyl, (C═O)—NHCH₃, NH₂, NH—(C═O)C₁₋₃alkyl, NH—(C═O)C₁₋₃haloalkyl, NH—(C═O)phenyl, NH—(C═O)cyclopropyl, and NH—(C═O)cyclopropyl wherein the cyclopropyl is substituted with one or two halo; 2,5-difluoro-4-pyridyl;

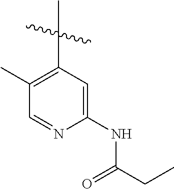 [SMILES: *c1cc(NC(=O)CC)ncc1C]

or 5-methylpyridin-2-amine;
(e) fused heteroaryl selected from the group consisting of: thieno[3,2-b]pyridinyl; pyrazolo[1,5-a]pyridin-5-yl optionally substituted with halo, C₁₋₃alkyl, or NH₂; 1H-pyrrolo[3,2-b]pyridinyl; 1H-pyrrolo[2,3-b]pyridinyl optionally substituted with one two or three members each independently selected from the group consisting of: halo, C₁₋₃alkyl, C₁₋₃haloalkyl, and CN; pyrazolo[1,5-a]pyrimidin-5-yl; [1,2,4]triazolo[1,5-a]pyridin-7-yl optionally substituted with C₁₋₃alkyl; 1H-pyrazolo[4,3-b]pyridin-7-yl optionally substituted with C₁₋₃alkyl; 1H-pyrazolo[3,4-b]pyridin-5-yl optionally substituted with C₁₋₃alkyl; 1H-pyrazolo[3,4-b]pyridinyl optionally substituted with one, two or three members each independently selected from the group consisting of: halo, C₁₋₃alkyl, C₁₋₃haloalkyl, and cyclopropyl; 2-methyl-2H-pyrazolo[4,3-b]pyridin-7-yl; 1-methyl-1H-pyrazolo[4,3-b]pyridin-7-yl; 2-methylpyrazolo[3,4-b]pyridin-4-yl; or 1H-pyrazolo[3,4-d]pyrimidin-4-yl optionally substituted with C₁₋₃alkyl; and
(f) 1,5-naphthyridin-4-yl optionally substituted with halo or OC₁₋₃alkyl;
R³ and R⁴ come together to form a group selected from the group consisting of:

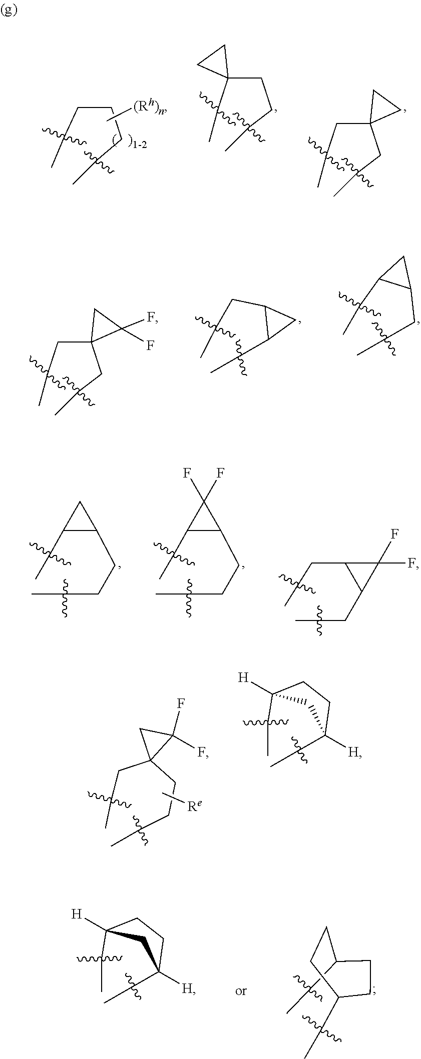

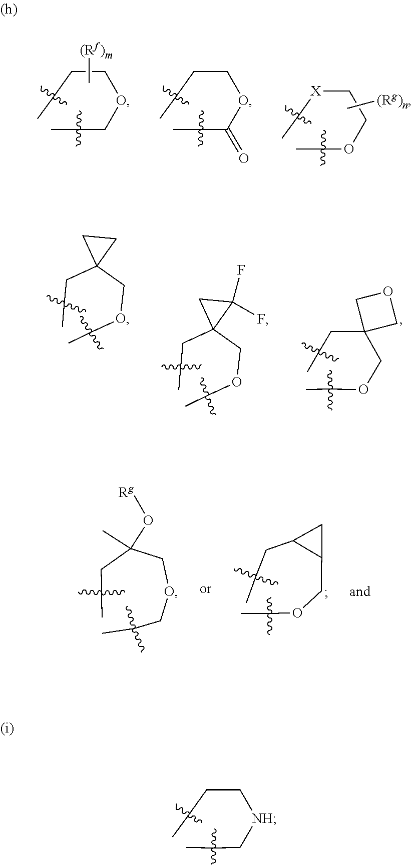

R^{f} is independently selected from the group consisting of: H, C₁₋₃alkyl, C₁₋₃haloalkyl, cyclopropyl, cyclobutyl, and two R^{f} members come together to form a C₃₋₆cycloalkyl wherein the C₃₋₆cycloalkyl is optionally substituted with one or two halo members;
R^{g} is H, halo, or C₁₋₃alkyl;
R^{h} is independently selected from the group consisting of H, halo, OH, C₁₋₃alkyl, CH₂OCH₃, CH₂CH₂OCH₃, C₁₋₃haloalkyl, CH₂OCHF₂, CH₂OCF₃, CN, an

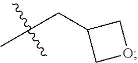 [SMILES: *CC1COC1]

X is selected from the group consisting of: a bond, CH₂, CH(CH₃), and CH₂CH₂;
m is 1, 2, 3 or 4; and
n is 1, 2, or 3;
and pharmaceutically acceptable salts, isotopes, N-oxides, solvates, and stereoisomers thereof.

An additional embodiment of the invention is a compound of Formula (I-1),

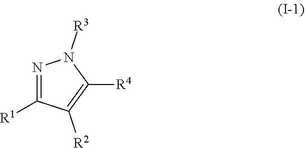

wherein
R¹ is selected from the group consisting of:
   (a) phenyl substituted with one or two halo members;

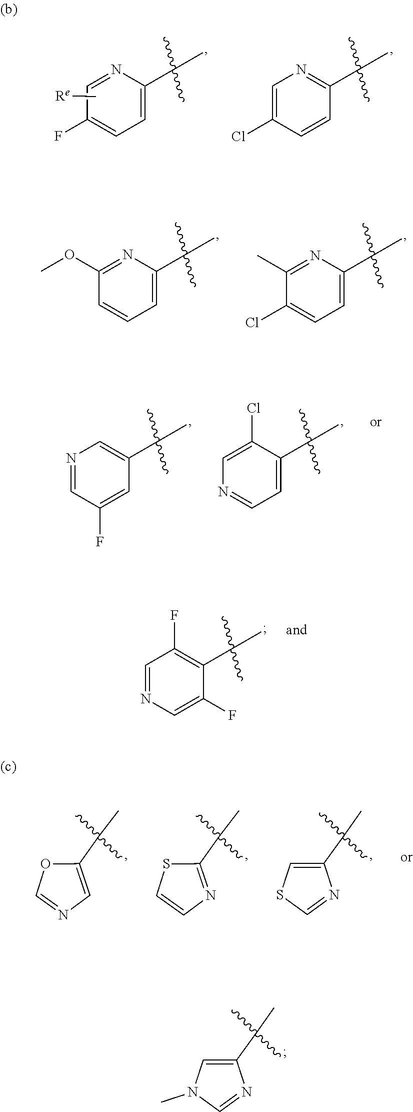

R² is selected from the group consisting of:

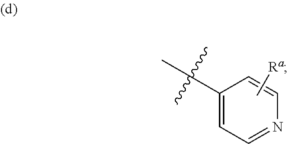

(e) fused heteroaryl selected from the group consisting of:

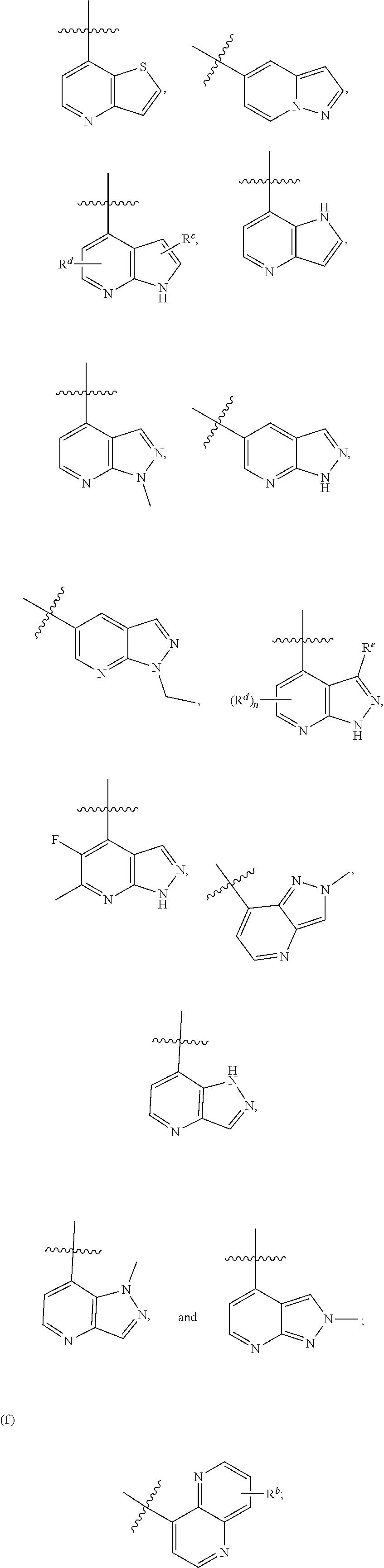

R³ and R⁴ come together to form a group selected from the group consisting of:

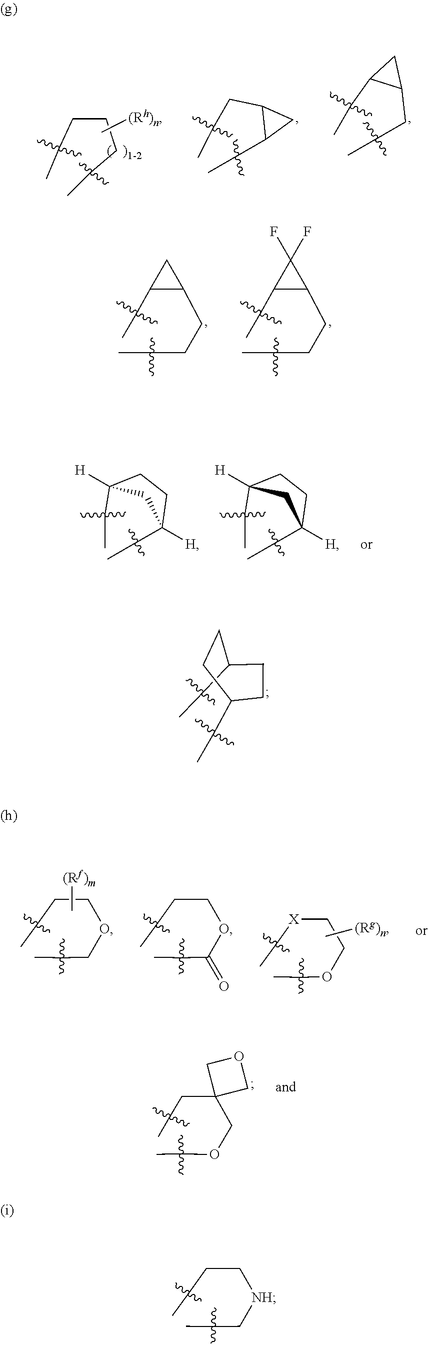

R^{a} is selected from the group consisting of: H, C₁₋₃haloalkyl, CH₂OH, (C═O)—NHCH₃, NH₂, NH—(C═O)C₁₋₃alkyl, NH—(C═O)C₁₋₃haloalkyl, NH—(C═O)cyclopropyl, NH—(C═O)cyclopropyl wherein the cyclopropyl is substituted with one or two halo, NH—(C═O)phenyl, and OC₁₋₃alkyl;
R^{b} is H, halo, or OCH₃;
R^{c} is H, C₁₋₃haloalkyl or CN;
R^{d} is independently selected from the group consisting of: H, halo, C₁₋₃alkyl, and cyclopropyl;
R^{e} is selected from the group consisting of: H, halo, and C₁₋₃alkyl;
R^{f} is independently selected from the group consisting of: H, C₁₋₃alkyl, C₁₋₃haloalkyl, cyclopropyl, cyclobutyl, and two R^{f} members come together to form cyclopropyl;
R^{g} is H or C₁₋₃alkyl;
R^{h} is independently selected from the group consisting of H, halo, C₁₋₃alkyl, CH₂OCH₃, and C₁₋₃haloalkyl;
X is selected from the group consisting of: CH₂, CH(CH₃), and CH₂CH₂;
m is 1, 2, 3 or 4; and
n is 1, 2, or 3;
and pharmaceutically acceptable salts, isotopes, N-oxides, solvates, and stereoisomers thereof.

An additional embodiment of the invention is a compound of Formula (I) wherein R¹ is

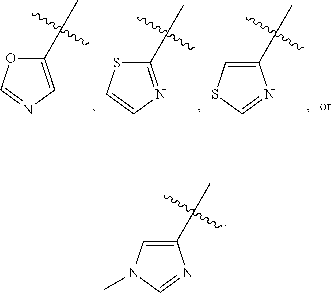

An additional embodiment of the invention is a compound of Formula (I) wherein R¹ is

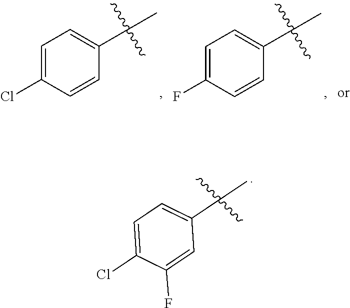 [SMILES: *c1ccc(Cl)cc1 ; *c1ccc(F)cc1 ; *c1ccc(Cl)c(F)c1]

An additional embodiment of the invention is a compound of Formula (I) wherein R¹ is

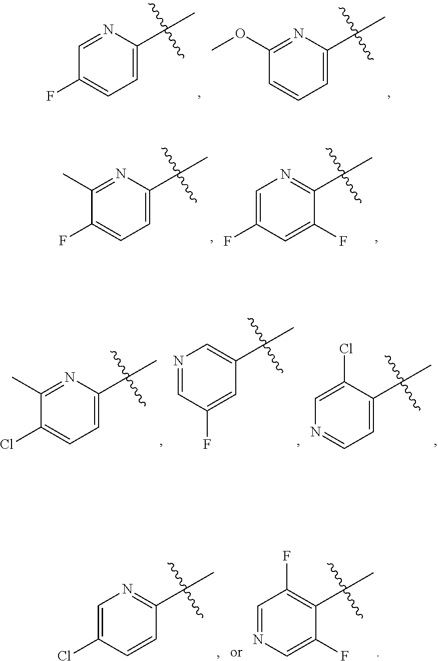

An additional embodiment of the invention is a compound of Formula (I) wherein R¹ is

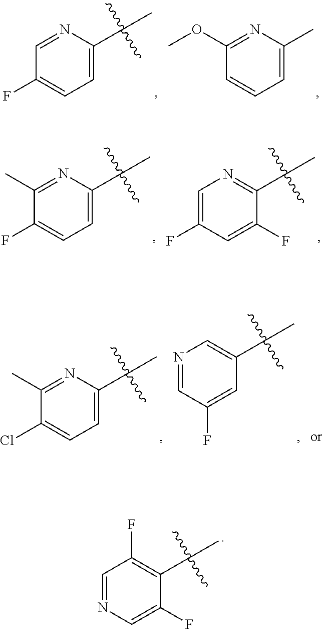

An additional embodiment of the invention is a compound of Formula (I) wherein R¹ is

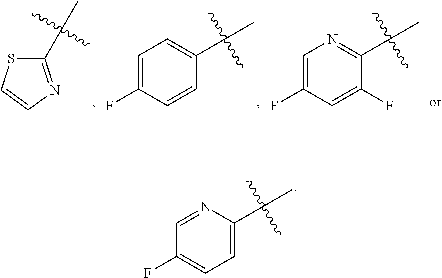 [SMILES: *c1nccs1 ; *c1ccc(F)cc1 ; *c1ncc(F)cc1F ; *c1ccc(F)cn1]

An additional embodiment of the invention is a compound of Formula (I) wherein R² is

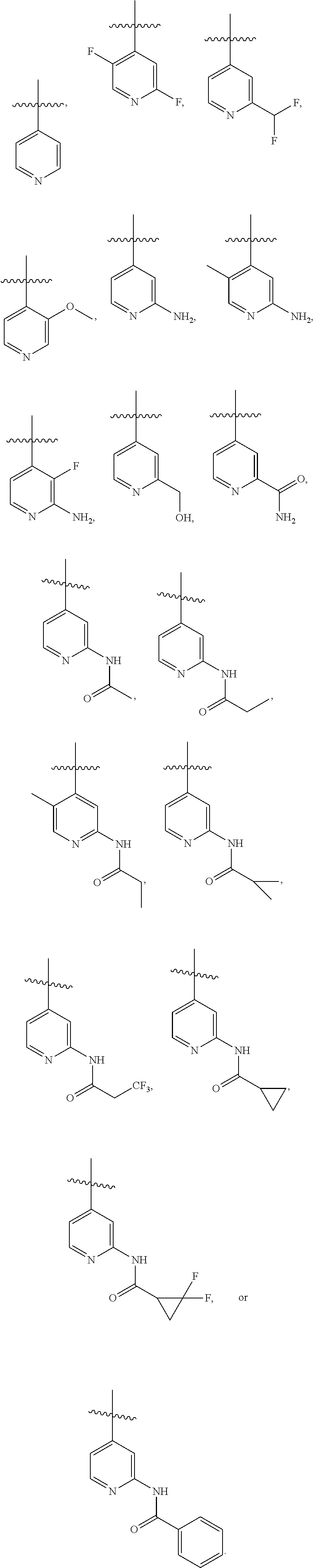

An additional embodiment of the invention is a compound of Formula (I) wherein R² is

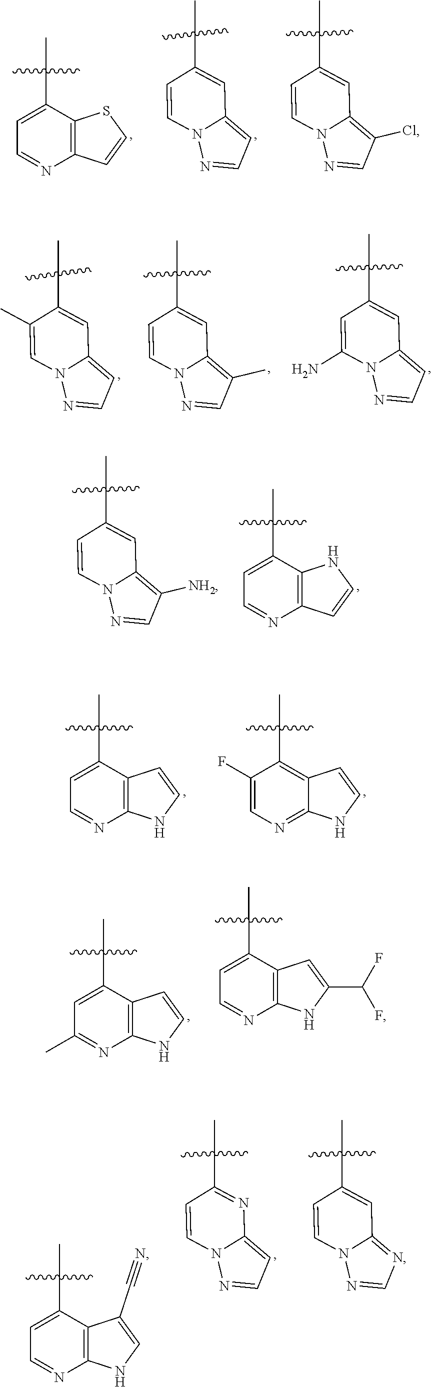

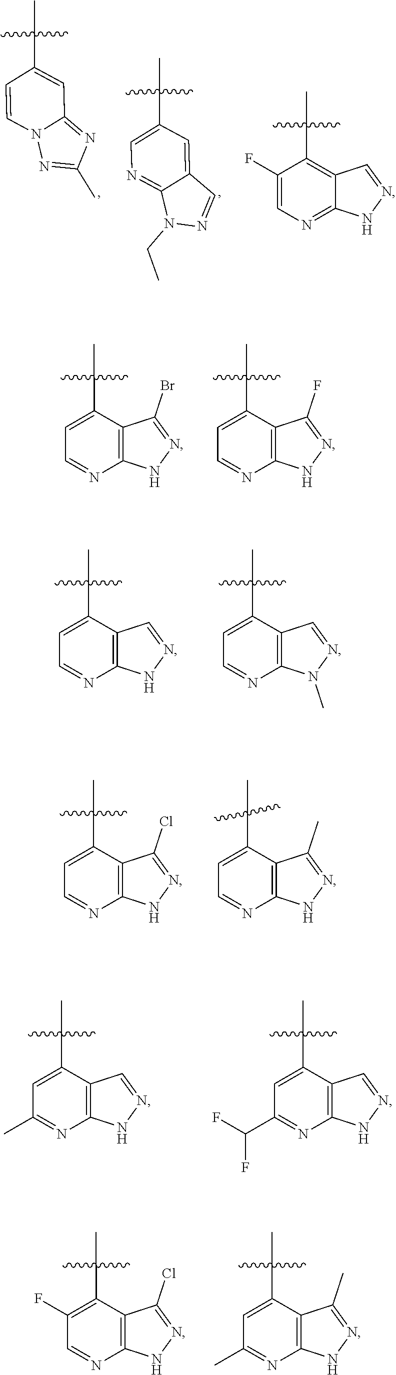

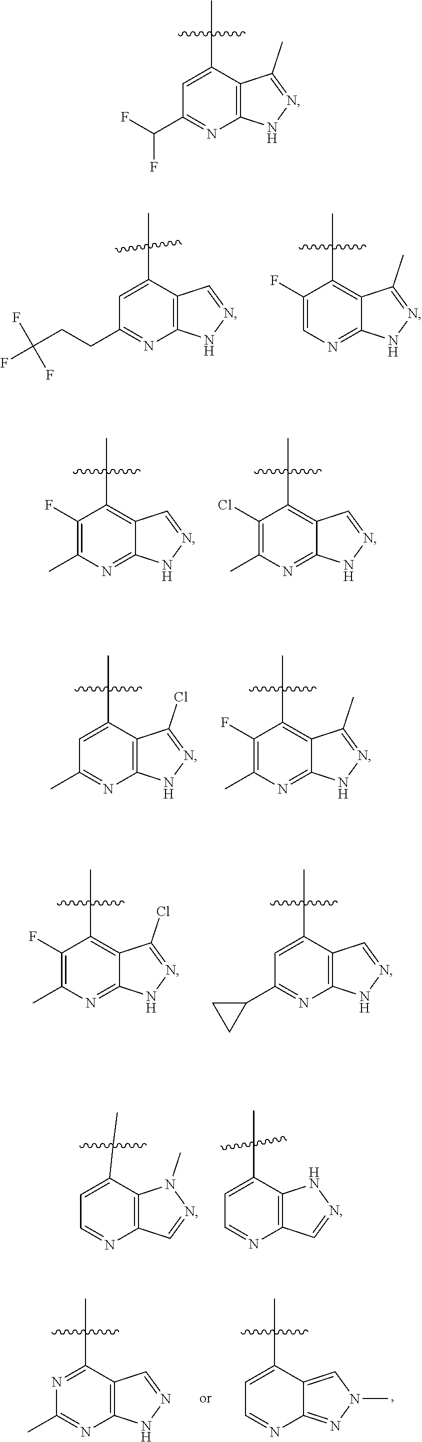

An additional embodiment of the invention is a compound of Formula (I) wherein R² is

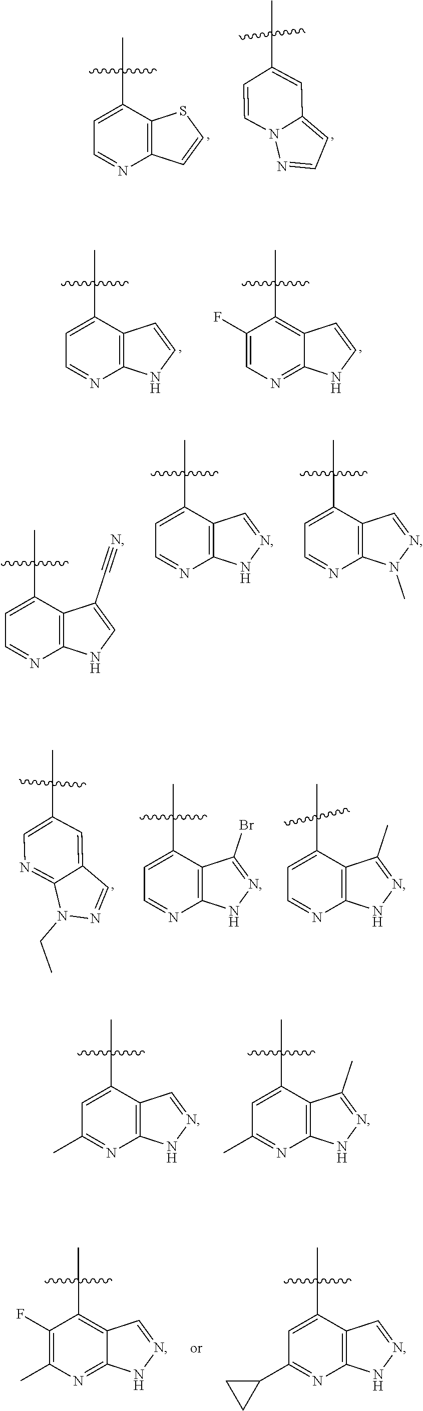

An additional embodiment of the invention is a compound of Formula (I) wherein R² is

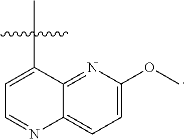 [SMILES: *c1ccnc2ccc(OC)nc12]

An additional embodiment of the invention is a compound of Formula (I) wherein R² is

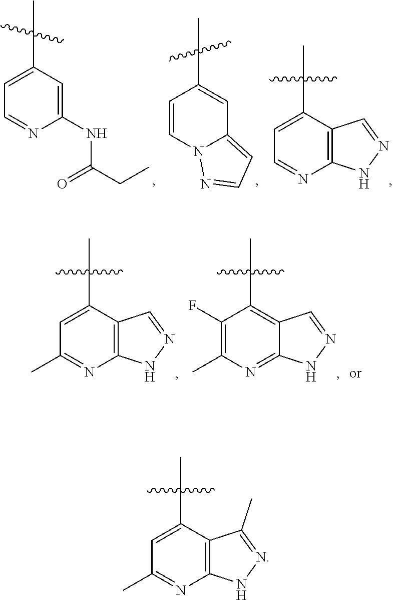

An additional embodiment of the invention is a compound of Formula (I) wherein R³-R⁴ is

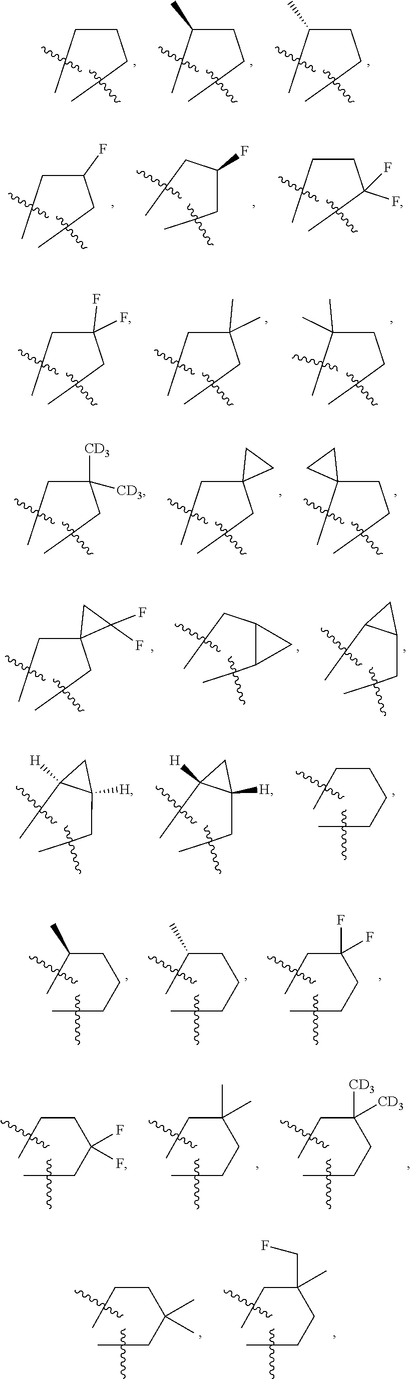

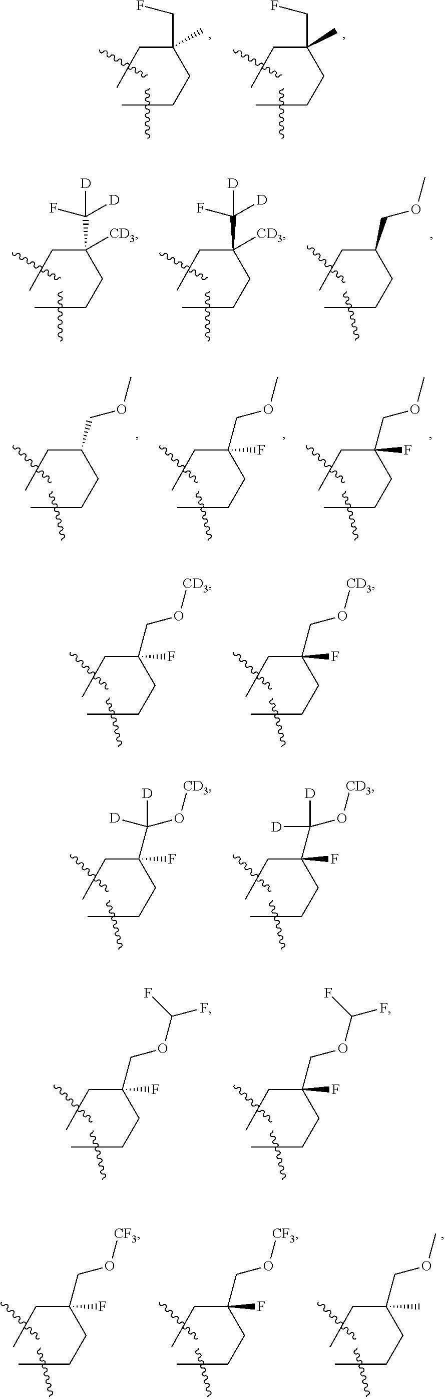

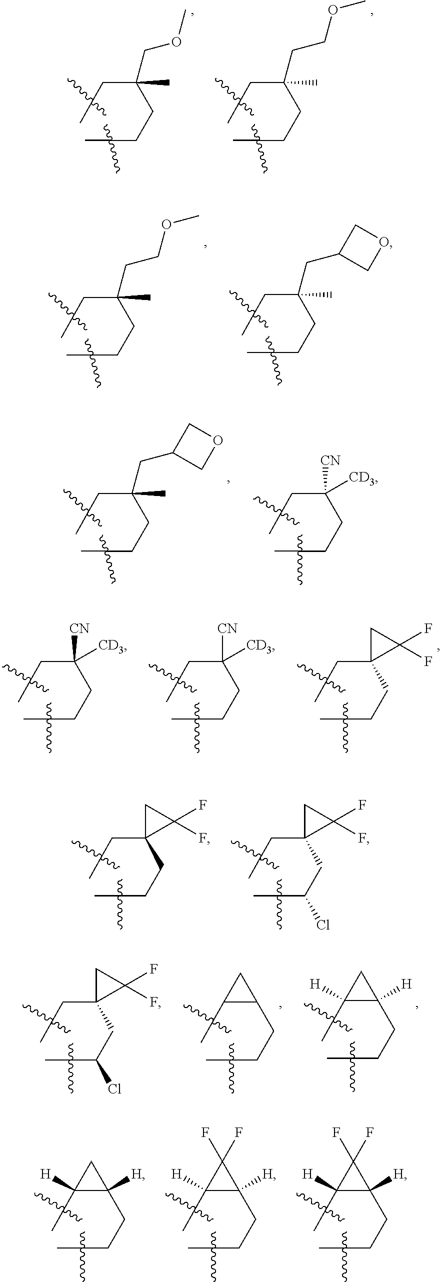

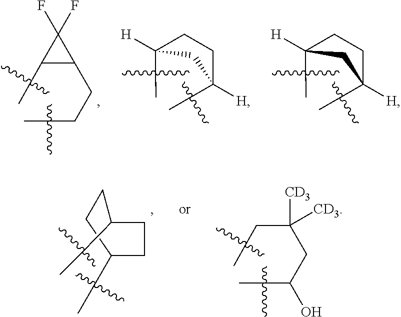

An additional embodiment of the invention is a compound of Formula (I) wherein R³-R⁴ is

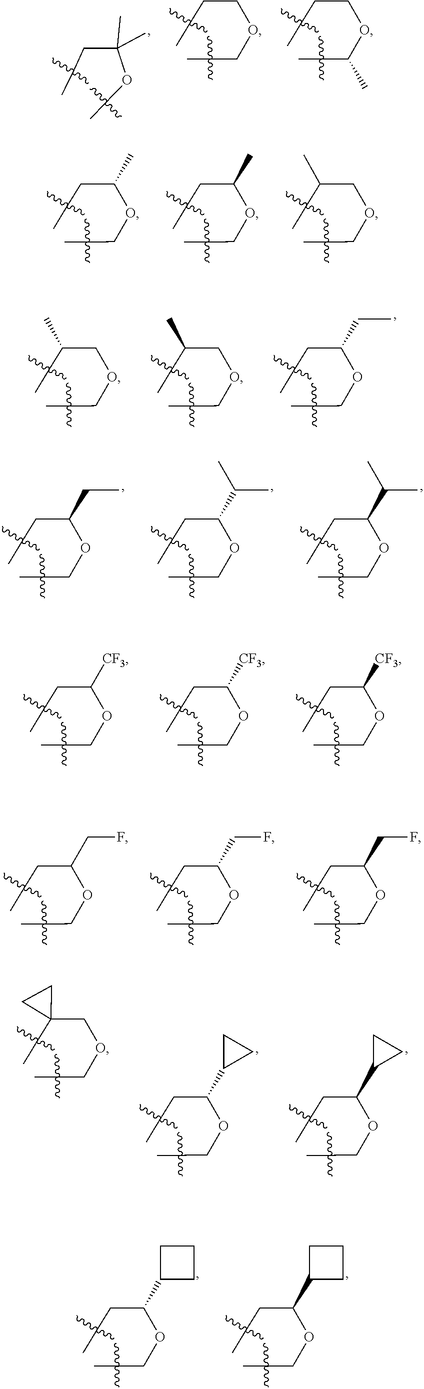

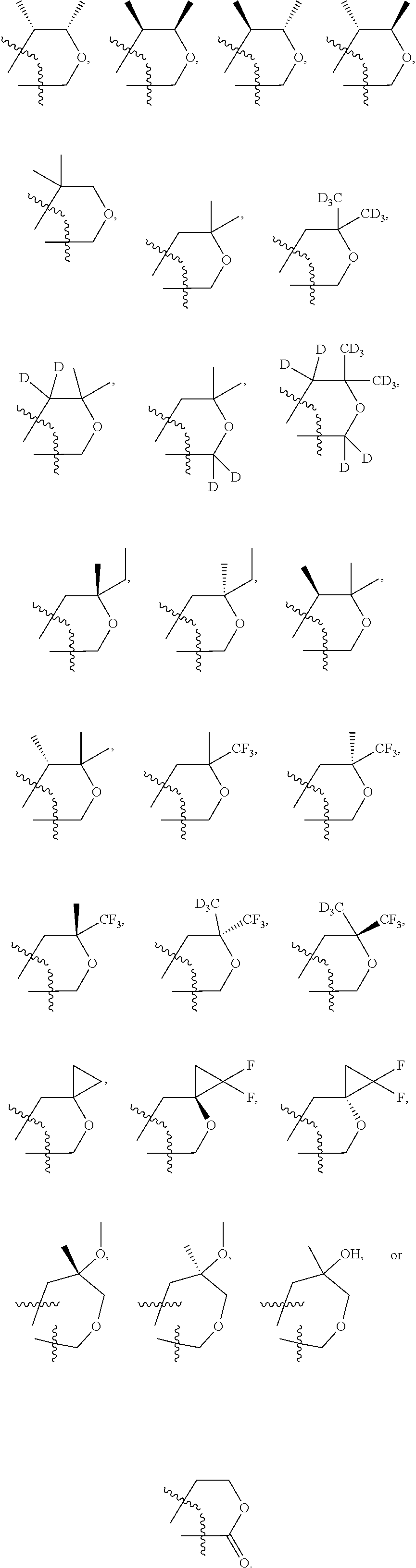

An additional embodiment of the invention is a compound of Formula (I) wherein R³-R⁴ is

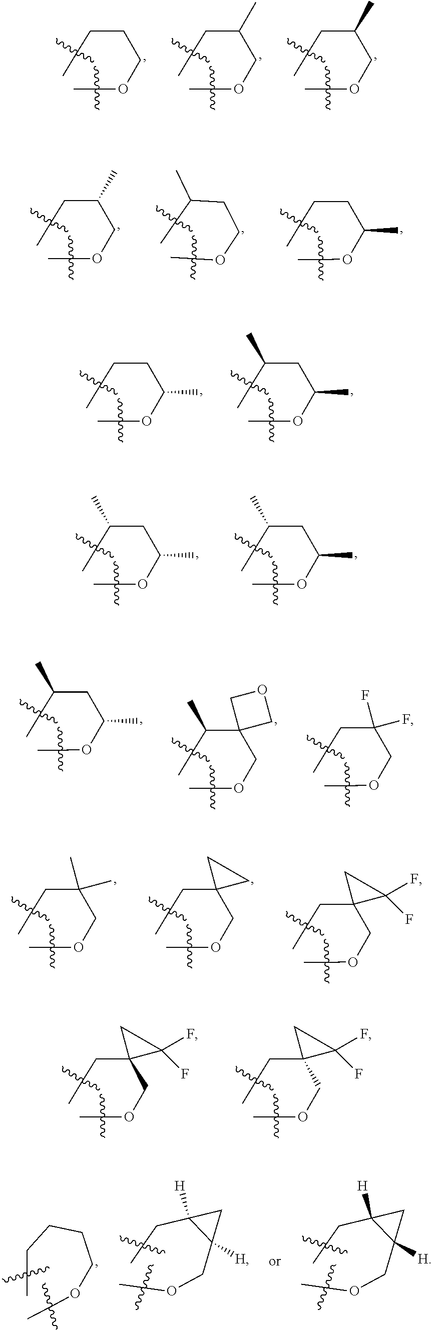

An additional embodiment of the invention is a compound of Formula (I) wherein R³-R⁴ is

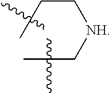 [SMILES: *CCNC*]

An additional embodiment of the invention is a compound of Formula (I) wherein R³-R⁴ is

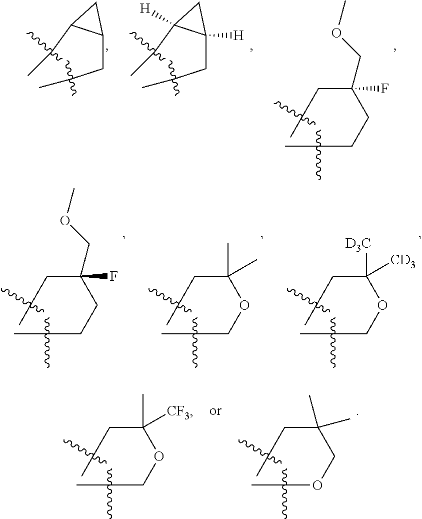

An additional embodiment of the invention is a compound of Formula (I) wherein m is 1.

An additional embodiment of the invention is a compound of Formula (I) wherein m is 2.

An additional embodiment of the invention is a compound of Formula (I) wherein m is 3.

An additional embodiment of the invention is a compound of Formula (I) wherein m is 4.

An additional embodiment of the invention is a compound of Formula (I) wherein n is 1.

An additional embodiment of the invention is a compound of Formula (I) wherein n is 2.

An additional embodiment of the invention is a compound of Formula (I) wherein n is 3.

A further embodiment of the current invention is a compound as shown below in Table 1

TABLE 1

| Ex # | Compound Name |
|---|---|
| 1 | 2-(5-Fluoro-2-pyridyl)-3-(1H-pyrrolo[2,3-b]pyridin-4-yl)-6,7-dihydro-4H- |
| | pyrazolo[5,1-c][1,4]oxazine; |
| 2 | 4-[2-(5-Fluoro-2-pyridyl)-4,5,6,7-tetrahydropyrazolo[1,5-a]pyrazin-3-yl]-6- |
| | methyl-1H-pyrazolo[3,4-b]pyridine; |
| 3 | 4-[2-(5-Fluoro-2-pyridyl)-4,5,6,7-tetrahydropyrazolo[1,5-a]pyridin-3-yl]-1H- |
| | pyrrolo[2,3-b]pyridine; |
| 4 | 4-(2-(4-Fluorophenyl)-5,6-dihydro-4H-pyrrolo[1,2-b]pyrazol-3-yl)-1H- |
| | pyrazolo[3,4-b]pyridine; |
| 5 | 4-(2-(4-Fluorophenyl)-5,6-dihydro-4H-pyrrolo[1,2-b]pyrazol-3-yl)-6-methyl- |
| | 1H-pyrazolo[3,4-b]pyridine; |
| 6 | 4-(2-(5-Fluoropyridin-2-yl)-5,6-dihydro-4H-pyrrolo[1,2-b]pyrazol-3-yl)-1H- |
| | pyrrolo[2,3-b]pyridine; |
| 7 | 4-[2-(5-Fluoro-2-pyridyl)-5,6-dihydro-4H-pyrrolo[1,2-b]pyrazol-3-yl]-1H- |
| | pyrazolo[3,4-b]pyridine; |
| 8 | 4-[2-(5-Fluoro-2-pyridyl)-5,6-dihydro-4H-pyrrolo[1,2-b]pyrazol-3-yl]-6- |
| | methyl-1H-pyrazolo[3,4-b]pyridine; |
| 9 | 5-Fluoro-4-(2-(5-fluoropyridin-2-yl)-5,6-dihydro-4H-pyrrolo[1,2-b]pyrazol-3- |
| | yl)-6-methyl-1H-pyrazolo[3,4-b]pyridine; |
| 10 | (S)-4-(5-Fluoro-2-(4-fluorophenyl)-5,6-dihydro-4H-pyrrolo[1,2-b]pyrazol-3- |
| | yl)pyridin-2-amine; |
| 11 | (S)-4-(5-Fluoro-2-(4-fluorophenyl)-5,6-dihydro-4H-pyrrolo[1,2-b]pyrazol-3- |
| | yl)-1H-pyrazolo[3,4-b]pyridine; |
| 12 | (S)-4-(5-Fluoro-2-(4-fluorophenyl)-5,6-dihydro-4H-pyrrolo[1,2-b]pyrazol-3- |
| | yl)-6-methyl-1H-pyrazolo[3,4-b]pyridine; |
| 13 | 4-(5-Fluoro-2-(5-fluoropyridin-2-yl)-5,6-dihydro-4H-pyrrolo[1,2-b]pyrazol-3- |
| | yl)-6-methyl-1H-pyrazolo[3,4-b]pyridine; |
| 14 | 4-(4,4-Difluoro-2-(5-fluoropyridin-2-yl)-5,6-dihydro-4H-pyrrolo[1,2-b]pyrazol- |
| | 3-yl)-1H-pyrazolo[3,4-b]pyridine; |
| 15 | 4-[(6S)-2-(4-Fluorophenyl)-6-methyl-5,6-dihydro-4H-pyrrolo[1,2-b]pyrazol-3- |
| | yl]-1H-pyrazolo[3,4-b]pyridine; |
| 16 | 4-[(6R)-2-(4-Fluorophenyl)-6-methyl-5,6-dihydro-4H-pyrrolo[1,2-b]pyrazol-3- |
| | yl]-1H-pyrazolo[3,4-b]pyridine; |
| 17 | 4-[(6S)-2-(4-Fluorophenyl)-6-methyl-5,6-dihydro-4H-pyrrolo[1,2-b]pyrazol-3- |
| | yl]-6-methyl-1H-pyrazolo[3,4-b]pyridine; |
| 18 | 4-[(6R)-2-(4-Fluorophenyl)-6-methyl-5,6-dihydro-4H-pyrrolo[1,2-b]pyrazol-3- |
| | yl]-6-methyl-1H-pyrazolo[3,4-b]pyridine; |
| 19 | 4-[(6S)-2-(5-Fluoro-2-pyridyl)-6-methyl-5,6-dihydro-4H-pyrrolo[1,2-b]pyrazol- |
| | 3-yl]-1H-pyrazolo[3,4-b]pyridine; |
| 20 | 4-[(6R)-2-(5-Fluoro-2-pyridyl)-6-methyl-5,6-dihydro-4H-pyrrolo[1,2- |
| | b]pyrazol-3-yl]-1H-pyrazolo[3,4-b]pyridine; |
| 21 | 4-[(6R)-2-(5-Fluoro-2-pyridyl)-6-methyl-5,6-dihydro-4H-pyrrolo[1,2- |
| | b]pyrazol-3-yl]-6-methyl-1H-pyrazolo[3,4-b]pyridine; |
| 22 | 8-[(6S)-2-(5-Fluoro-2-pyridyl)-6-methyl-5,6-dihydro-4H-pyrrolo[1,2-b]pyrazol- |
| | 3-yl]-2-methoxy-1,5-naphthyridine; |
| 23 | (Racemic) 2-(5-Fluoropyridin-2-yl)-3-(1H-pyrazolo[3,4-b]pyridin-4-yl)- |
| | 3b,4,4a,5-tetrahydrocyclopropa[3,4]pyrrolo[1,2-b]pyrazole; |
| 24 | (Racemic) 2-(5-Fluoropyridin-2-yl)-3-(6-methyl-1H-pyrazolo[3,4-b]pyridin-4- |
| | yl)-3b,4,4a,5-tetrahydrocyclopropa[3,4]pyrrolo[1,2-b]pyrazole; |
| 25 | (4aR,5aR)-2-(5-Fluoropyridin-2-yl)-3-(1H-pyrazolo[3,4-b]pyridin-4-y])- |
| | 4,4a,5,5a-tetrahydrocyclopropa[4,5]pyrrolo[1,2-b]pyrazole; |
| 26 | (4aS,5aS)-2-(5-Fluoropyridin-2-yl)-3-(1H-pyrazolo[3,4-b]pyridin-4-yl)- |
| | 4,4a,5,5a-tetrahydrocyclopropa[4,5]pyrrolo[1,2-b]pyrazole; |
| 27 | (4aR,5aR)-2-(5-Fluoropyridin-2-yl)-3-(6-methyl-1H-pyrazolo[3,4-b]pyridin-4- |
| | yl)-4,4a,5,5a-tetrahydrocyclopropa[4,5]pyrrolo[1,2-b]pyrazole; |
| 28 | (4aS,5aS)-2-(5-Fluoropyridin-2-yl)-3-(6-methyl-1H-pyrazolo[3,4-b]pyridin-4- |
| | yl)-4,4a,5,5a-tetrahydrocyclopropa[4,5]pyrrolo[1,2-b]pyrazole; |
| 29 | 2-(3-(1H-Pyrazolo[3,4-b]pyridin-4-yl)-4,5,6,7-tetrahydropyrazolo[1,5- |
| | a]pyridin-2-yl)thiazole; |
| 30 | 2-(3-(6-Methyl-1H-pyrazolo[3,4-b]pyridin-4-yl)-4,5,6,7- |
| | tetrahydropyrazolo[1,5-a]pyridin-2-yl)thiazole; |
| 31 | 4-(3-(1H-Pyrazolo[3,4-b]pyridin-4-yl)-4,5,6,7-tetrahydropyrazolo[1,5- |
| | a]pyridin-2-yl)thiazole; |
| 32 | 4-(3-(6-Methyl-1H-pyrazolo[3,4-b]pyridin-4-yl)-4,5,6,7- |
| | tetrahydropyrazolo[1,5-a]pyridin-2-yl)thiazole; |
| 33 | 4-[2-(4-Fluorophenyl)-4,5,6,7-tetrahydropyrazolo[1,5-a]pyridin-3-yl]-1H- |
| | pyrrolo[2,3-b]pyridine; |
| 34 | 5-Fluoro-4-[2-(5-fluoro-2-pyridyl)-4,5,6,7-tetrahydropyrazolo[1,5-a]pyridin-3- |
| | yl]-1H-pyrrolo[2,3-b]pyridine; |
| 35 | pyrazolo[3,4-b]pyridine; |
| 36 | 4-[2-(5-Fluoro-2-pyridyl)-4,5,6,7-tetrahydropyrazolo[1,5-a]pyridin-3-yl]-6- |
| | methyl-1H-pyrazolo[3,4-b]pyridine; |
| 37 | 4-[2-(3,5-Difluoro-2-pyridyl)-4,5,6,7-tetrahydropyrazolo[1,5-a]pyridin-3-yl]-6- |
| | methyl-1H-pyrazolo[3,4-b]pyridine; |
| 38 | 1H-pyrazolo[3,4-b]pyridine; |
| 39 | (*R)-4-[2-(4-Fluorophenyl)-7-methyl-4,5,6,7-tetrahydropyrazolo[1,5-a]pyridin- |
| | 3-yl]-1H-pyrazolo[3,4-b]pyridine; |
| 40 | (*S)-4-[2-(4-Fluorophenyl)-7-methyl-4,5,6,7-tetrahydropyrazolo[1,5-a]pyridin- |
| | 3-yl]-1H-pyrazolo[3,4-b]pyridine; |
| 41 | 4-(6,6-Difluoro-2-(4-fluorophenyl)-4,5,6,7-tetrahydropyrazolo[1,5-a]pyridin-3- |
| | yl)pyridin-2-amine; |
| 42 | 4-(6,6-Difluoro-2-(4-fluorophenyl)-4,5,6,7-tetrahydropyrazolo[1,5-a]pyridin-3- |
| | yl)-1H-pyrazolo[3,4-b]pyridine; |
| 43 | 4-[6,6-Difluoro-2-(4-fluorophenyl)-5,7-dihydro-4H-pyrazolo[1,5-a]pyridin-3- |
| | yl]-6-methyl-1H-pyrazolo[3,4-b]pyridine; |
| 44 | 4-(6,6-Difluoro-2-(5-fluoropyridin-2-yl)-4,5,6,7-tetrahydropyrazolo[1,5- |
| | a]pyridin-3-yl)-1H-pyrazolo[3,4-b]pyridine; |
| 45 | 4-(6,6-Difluoro-2-(5-fluoropyridin-2-yl)-4,5,6,7-tetrahydropyrazolo[1,5- |
| | a]pyridin-3-yl)-6-methyl-1H-pyrazolo[3,4-b]pyridine; |
| 46 | 4-(5,5-Difluoro-2-(4-fluorophenyl)-4,5,6,7-tetrahydropyrazolo[1,5-a]pyridin-3- |
| | yl)-1H-pyrazolo[3,4-b]pyridine; |
| 47 | 4-(5,5-Difluoro-2-(4-fluorophenyl)-4,5,6,7-tetrahydropyrazolo[1,5-a]pyridin-3- |
| | yl)-6-methyl-1H-pyrazolo[3,4-b]pyridine; |
| 48 | 4-[5,5-Difluoro-2-(5-fluoro-2-pyridyl)-6,7-dihydro-4H-pyrazolo[1,5-a]pyridin- |
| | 3-yl]-1H-pyrazolo[3,4-b]pyridine; |
| 49 | 4-[2-(5-Fluoro-2-pyridyl)-6,6-dimethyl-5,7-dihydro-4H-pyrazolo[1,5-a]pyridin- |
| | 3-yl]-1H-pyrazolo[3,4-b]pyridine; |
| 50 | 4-(2-(5-Fluoropyridin-2-yl)-5,5-dimethyl-4,5,6,7-tetrahydropyrazolo[1,5- |
| | alpyridin-3-yl)-1H-pyrazolo[3,4-b]pyridine; |
| 51 | (*S)-4-[6-(Fluoromethyl)-2-(5-fluoro-2-pyridyl)-6-methyl-5,7-dihydro-4H- |
| | pyrazolo[1,5-a]pyridin-3-yl]-1H-pyrazolo[3,4-b]pyridine; |
| 52 | (*R)-4-[6-(Fluoromethyl)-2-(5-fluoro-2-pyridyl)-6-methyl-5,7-dihydro-4H- |
| | pyrazolo[1,5-a]pyridin-3-yl]-1H-pyrazolo[3,4-b]pyridine; |
| 53 | (*S)-4-(6-Fluoro-2-(5-fluoropyridin-2-yl)-6-(methoxymethyl)-4,5,6,7- |
| | tetrahydropyrazolo[1,5-a]pyridin-3-yl)-6-methyl-1H-pyrazolo[3,4-b]pyridine; |
| 54 | (*R)-4-(6-Fluoro-2-(5-fluoropyridin-2-yl)-6-(methoxymethyl)-4,5,6,7- |
| | tetrahydropyrazolo[1,5-a]pyridin-3-yl)-6-methyl-1H-pyrazolo[3,4-b]pyridine; |
| 55 | (*S)-4-(2-(5-Fluoropyridin-2-yl)-6-(methoxymethyl)-6-methyl-4,5,6,7- |
| | tetrahydropyrazolo[1,5-a]pyridin-3-yl)-6-methyl-1H-pyrazolo[3,4-b]pyridine; |
| 56 | (*R)-4-(2-(5-Fluoropyridin-2-yl)-6-(methoxymethyl)-6-methyl-4,5,6,7- |
| | tetrahydropyrazolo[1,5-a]pyridin-3-yl)-6-methyl-1H-pyrazolo[3,4-b]pyridine; |
| 57 | (Racemic) 2-(5-Fluoropyridin-2-yl)-3-(6-methyl-1H-pyrazolo[3,4-b]pyridin-4- |
| | yl)-5,5a,6,6a-tetrahydro-4H-cyclopropa[e]pyrazolo[1,5-a]pyridine; |
| 58 | (Racemic) 6,6-Difluoro-2-(5-fluoropyridin-2-yl)-3-(1H-pyrazolo[3,4-b]pyridin- |
| | 4-yl)-5,5a,6,6a-tetrahydro-4H-cyclopropa[e]pyrazolo[1,5-a]pyridine; |
| 59 | (Racemic) 6,6-Difluoro-2-(5-fluoropyridin-2-yl)-3-(6-methyl-1H-pyrazolo[3,4- |
| | b]pyridin-4-yl)-5,5a,6,6a-tetrahydro-4H-cyclopropa[e]pyrazolo[1,5-a]pyridine; |
| 60 | (4*R,7*S)-2-(4-Fluorophenyl)-3-(1H-pyrazolo[3,4-b]pyridin-4-yl)-4,5,6,7- |
| | tetrahydro-4,7-methanopyrazolo[1,5-a]pyridine; |
| 61 | (4*S,7*R)-2-(4-Fluorophenyl)-3-(1H-pyrazolo[3,4-b]pyridin-4-yl)-4,5,6,7- |
| | tetrahydro-4,7-methanopyrazolo[1,5-a]pyridine; |
| 62 | (4*R,7*S)-2-(5-Fluoropyridin-2-yl)-3-(1H-pyrazolo[3,4-b]pyridin-4-yl)-4,5,6,7- |
| | tetrahydro-4,7-methanopyrazolo[1,5-a]pyridine; |
| 63 | (4*S,7*R)-2-(5-Fluoropyridin-2-yl)-3-(1H-pyrazolo[3,4-b]pyridin-4-yl)-4,5,6,7- |
| | tetrahydro-4,7-methanopyrazolo[1,5-a]pyridine; |
| 64 | 2-(4-Fluorophenyl)-3-(1H-pyrazolo[3,4-b]pyridin-4-yl)-4,5,6,7-tetrahydro-4,7- |
| | ethanopyrazolo[1,5-a]pyridine; |
| 65 | 2-(5-Fluoropyridin-2-yl)-3-(1H-pyrazolo[3,4-b]pyridin-4-yl)-4,5,6,7-tetrahydro- |
| | 4,7-ethanopyrazolo[1,5-a]pyridine; |
| 66 | 2-(4-Fluorophenyl)-3-(6-methyl-1H-pyrazolo[3,4-b]pyridin-4-yl)-4,5,6,7- |
| | tetrahydro-4,7-ethanopyrazolo[1,5-a]pyridine; |
| 67 | 2-(4-Chlorophenyl)-3-(1H-pyrazolo[3,4-b]pyridin-4-yl)-6,7-dihydro-4H- |
| | pyrazolo[5,1-c][1,4]oxazine; |
| 68 | 2-(4-Chlorophenyl)-3-(6-methyl-1H-pyrazolo[3,4-b]pyridin-4-yl)-6,7-dihydro- |
| | 4H-pyrazolo[5,1-c][1,4]oxazine; |
| 69 | 2-(4-Fluorophenyl)-3-(1H-pyrrolo[2,3-b]pyridin-4-yl)-6,7-dihydro-4H- |
| | pyrazolo[5,1-c][1,4]oxazine; |
| 70 | 2-(4-Fluorophenyl)-3-(1H-pyrazolo[3,4-b]pyridin-4-yl)-6,7-dihydro-4H- |
| | pyrazolo[5,1-c][1,4]oxazine; |
| 71 | 2-(4-Fluorophenyl)-3-(6-methyl-1H-pyrazolo[3,4-b]pyridin-4-y])-6,7-dihydro- |
| | 4H-pyrazolo[5,1-c][1,4]oxazine; |
| 72 | 2-(4-Chloro-3-fluoro-phenyl)-3-(1H-pyrazolo[3,4-b]pyridin-4-yl)-6,7-dihydro- |
| | 4H-pyrazolo[5,1-c][1,4]oxazine; |
| 73 | 2-(4-Chloro-3-fluoro-phenyl)-3-(6-methyl-1H-pyrazolo[3,4-b]pyridin-4-yl)-6,7- |
| | dihydro-4H-pyrazolo[5,1-c][1,4]oxazine; |
| 74 | 2-(5-Fluoro-2-pyridyl)-3-(4-pyridyl)-6,7-dihydro-4H-pyrazolo[5,1- |
| | c][1,4]oxazine; |
| 75 | 3-(2-(Difluoromethyl)-1H-pyrrolo[2,3-b]pyridin-4-yl)-2-(5-fluoropyridin-2-yl)- |
| | 6,7-dihydro-4H-pyrazolo[5,1-c][1,4]oxazine; |
| 76 | 2-(5-Fluoro-3-pyridyl)-3-(1H-pyrazolo[3,4-b]pyridin-4-yl)-6,7-dihydro-4H- |
| | pyrazolo[5,1-c][1,4]oxazine; |
| 77 | 2-(5-Fluoro-3-pyridyl)-3-(6-methyl-1H-pyrazolo[3,4-b]pyridin-4-yl)-6,7- |
| | dihydro-4H-pyrazolo[5,1-c][1,4]oxazine; |
| 78 | 2-(5-Fluoro-2-pyridyl)-3-(1H-pyrazolo[3,4-b]pyridin-4-yl)-6,7-dihydro-4H- |
| | pyrazolo[5,1-c][1,4]oxazine; |
| 79 | 2-(5-Fluoro-2-pyridyl)-3-(6-methyl-1H-pyrazolo[3,4-b]pyridin-4-yl)-6,7- |
| | dihydro-4H-pyrazolo[5,1-c][1,4]oxazine; |
| 80 | (R)-2-(5-Fluoropyridin-2-yl)-4-methyl-3-(1H-pyrrolo[2,3-b]pyridin-4-yl)-6,7- |
| | dihydro-4H-pyrazolo[5,1-c][1,4]oxazine; |
| 81 | (*S)-2-(4-Fluorophenyl)-6-methyl-3-(1H-pyrazolo[3,4-b]pyridin-4-yl)-6,7- |
| | dihydro-4H-pyrazolo[5,1-c][1,4]oxazine; |
| 82 | (*R)-2-(4-Fluorophenyl)-6-methyl-3-(1H-pyrazolo[3,4-b]pyridin-4-yl)-6,7- |
| | dihydro-4H-pyrazolo[5,1-c][1,4]oxazine; |
| 83 | (*S)-2-(4-Fluorophenyl)-6-methyl-3-(6-methyl-1H-pyrazolo[3,4-b]pyridin-4- |
| | yl)-6,7-dihydro-4H-pyrazolo[5,1-c][1,4]oxazine; |
| 84 | (*R)-2-(4-Fluorophenyl)-6-methyl-3-(6-methyl-1H-pyrazolo[3,4-b]pyridin-4- |
| | yl)-6,7-dihydro-4H-pyrazolo[5,1-c][1,4]oxazine; |
| 85 | (*R)-2-(5-Fluoro-2-pyridyl)-6-methyl-3-(1H-pyrazolo[3,4-b]pyridin-4-yl)-6,7- |
| | dihydro-4H-pyrazolo[5,1-c][1,4]oxazine; |
| 86 | (*S)-2-(5-Fluoro-2-pyridyl)-6-methyl-3-(1H-pyrazolo[3,4-b]pyridin-4-yl)-6,7- |
| | dihydro-4H-pyrazolo[5,1-c][1,4]oxazine; |
| 87 | (*R)-2-(5-Fluoro-2-pyridyl)-6-methyl-3-(6-methyl-1H-pyrazolo[3,4-b]pyridin- |
| | 4-yl)-6,7-dihydro-4H-pyrazolo[5,1-c][1,4]oxazine; |
| 88 | (*S)-2-(5-Fluoro-2-pyridyl)-6-methyl-3-(6-methyl-1H-pyrazolo[3,4-b]pyridin- |
| | 4-yl)-6,7-dihydro-4H-pyrazolo[5,1-c][1,4]oxazine; |
| 89 | (S)-2-(5-Fluoropyridin-2-yl)-6-methyl-3-(6-methyl-1H-pyrazolo[3,4-b]pyridin- |
| | 4-yl)-6,7-dihydro-4H-pyrazolo[5,1-c][1,4]oxazine; |
| 90 | (4-(2-(4-Fluorophenyl)-7-methyl-6,7-dihydro-4H-pyrazolo[5,1-c][1,4]oxazin-3- |
| | yl)pyridin-2-yl)methanol; |
| 91 | (*S)-2-(4-Fluorophenyl)-7-methyl-3-(1H-pyrazolo[3,4-b]pyridin-4-yl)-6,7- |
| | dihydro-4H-pyrazolo[5,1-c][1,4]oxazine; |
| 92 | (*R)-2-(4-Fluorophenyl)-7-methyl-3-(1H-pyrazolo[3,4-b]pyridin-4-yl)-6,7- |
| | dihydro-4H-pyrazolo[5,1-c][1,4]oxazine; |
| 93 | (*S)-2-(4-Fluorophenyl)-7-methyl-3-(6-methyl-1H-pyrazolo[3,4-b]pyridin-4- |
| | yl)-6,7-dihydro-4H-pyrazolo[5,1-c][1,4]oxazine; |
| 94 | (*R)-2-(4-Fluorophenyl)-7-methyl-3-(6-methyl-1H-pyrazolo[3,4-b]pyridin-4- |
| | yl)-6,7-dihydro-4H-pyrazolo[5,1-c][1,4]oxazine; |
| 95 | (*R)-6-Ethyl-2-(5-fluoropyridin-2-yl)-3-(1H-pyrazolo[3,4-b]pyridin-4-yl)-6,7- |
| | dihydro-4H-pyrazolo[5,1-c][1,4]oxazine; |
| 96 | (*S)-6-Ethyl-2-(5-fluoropyridin-2-yl)-3-(1H-pyrazolo[3,4-b]pyridin-4-yl)-6,7- |
| | dihydro-4H-pyrazolo[5,1-c][1,4]oxazine; |
| 97 | (*R)-2-(5-Fluoropyridin-2-yl)-6-isopropyl-3-(1H-pyrazolo[3,4-b]pyridin-4-yl)- |
| | 6,7-dihydro-4H-pyrazolo[5,1-c][1,4]oxazine; |
| 98 | (*S)-2-(5-Fluoropyridin-2-yl)-6-isopropyl-3-(1H-pyrazolo[3,4-b]pyridin-4-yl)- |
| | 6,7-dihydro-4H-pyrazolo[5,1-c][1,4]oxazine; |
| 99 | 2-(4-Fluorophenyl)-3-(1H-pyrazolo[3,4-b]pyridin-4-yl)-6-(trifluoromethyl)-6,7- |
| | dihydro-4H-pyrazolo[5,1-c][1,4]oxazine; |
| 100 | (S)-2-(4-Fluorophenyl)-3-(1H-pyrazolo[3,4-b]pyridin-4-yl)-6-(trifluoromethyl)- |
| | 6,7-dihydro-4H-pyrazolo[5,1-c][1,4]oxazine; |
| 101 | (R)-2-(4-Fluorophenyl)-3-(1H-pyrazolo[3,4-b]pyridin-4-yl)-6-(trifluoromethyl)- |
| | 6,7-dihydro-4H-pyrazolo[5,1-c][1,4]oxazine; |
| 102 | 2-(5-Fluoropyridin-2-yl)-3-(1H-pyrazolo[3,4-b]pyridin-4-yl)-6- |
| | (trifluoromethyl)-6,7-dihydro-4H-pyrazolo[5,1-c][1,4]oxazine; |
| 103 | 6-(Fluoromethyl)-2-(4-fluorophenyl)-3-(1H-pyrazolo[3,4-b]pyridin-5-yl)-6,7- |
| | dihydro-4H-pyrazolo[5,1-c][1,4]oxazine; |
| 104 | (*S)-6-(Fluoromethyl)-2-(4-fluorophenyl)-3-(1H-pyrazolo[3,4-b]pyridin-4-y])- |
| | 6,7-dihydro-4H-pyrazolo[5,1-c][1,4]oxazine; |
| 105 | (*R)-6-(Fluoromethyl)-2-(4-fluorophenyl)-3-(1H-pyrazolo[3,4-b]pyridin-4-yl)- |
| | 6,7-dihydro-4H-pyrazolo[5,1-c][1,4]oxazine; |
| 106 | 2′-(4-Fluorophenyl)-3′-(1H-pyrazolo[3,4-b]pyridin-4-yl)-4′H,6′H- |
| | spiro[cyclopropane-1,7′-pyrazolo[5,1-c][1,4]oxazine]; |
| 107 | 2′-(4-Fluorophenyl)-3′-(6-methyl-1H-pyrazolo[3,4-b]pyridin-4-yl)-4′H,6′H- |
| | spiro[cyclopropane-1,7′-pyrazolo[5,1-c][1,4]oxazine]; |
| 108 | (*R)-6-Cyclopropyl-2-(5-fluoropyridin-2-yl)-3-(1H-pyrazolo[3,4-b]pyridin-4- |
| | yl)-6,7-dihydro-4H-pyrazolo[5,1-c][1,4]oxazine; |
| 109 | (*S)-6-Cyclopropyl-2-(5-fluoropyridin-2-yl)-3-(1H-pyrazolo[3,4-b]pyridin-4- |
| | yl)-6,7-dihydro-4H-pyrazolo[5,1-c][1,4]oxazine; |
| 110 | (*R)-6-Cyclobutyl-2-(5-fluoropyridin-2-yl)-3-(1H-pyrazolo[3,4-b]pyridin-4-yl)- |
| | 6,7-dihydro-4H-pyrazolo[5,1-c][1,4]oxazine; |
| 111 | (*S)-6-Cyclobutyl-2-(5-fluoropyridin-2-yl)-3-(1H-pyrazolo[3,4-b]pyridin-4-y])- |
| | 6,7-dihydro-4H-pyrazolo[5,1-c][1,4]oxazine; |
| 112 | (6*R,7*S)-2-(4-Fluorophenyl)-6,7-dimethyl-3-(1H-pyrazolo[3,4-b]pyridin-4- |
| | yl)-6,7-dihydro-4H-pyrazolo[5,1-c][1,4]oxazine; |
| 113 | (6*S,7*R)-2-(4-Fluorophenyl)-6,7-dimethyl-3-(1H-pyrazolo[3,4-b]pyridin-4- |
| | yl)-6,7-dihydro-4H-pyrazolo[5,1-c][1,4]oxazine; |
| 114 | (6*R,7*R)-2-(4-Fluorophenyl)-6,7-dimethyl-3-(1H-pyrazolo[3,4-b]pyridin-4- |
| | yl)-6,7-dihydro-4H-pyrazolo[5,1-c][1,4]oxazine; |
| 115 | (6*S,7*S)-2-(4-Fluorophenyl)-6,7-dimethyl-3-(1H-pyrazolo[3,4-b]pyridin-4- |
| | yl)-6,7-dihydro-4H-pyrazolo[5,1-c][1,4]oxazine; |
| 116 | (6*R,7*S)-2-(5-Fluoropyridin-2-yl)-6,7-dimethyl-3-(1H-pyrazolo[3,4-b]pyridin- |
| | 4-yl)-6,7-dihydro-4H-pyrazolo[5,1-c][1,4]oxazine; |
| 117 | (6*S,7*R)-2-(5-Fluoropyridin-2-yl)-6,7-dimethyl-3-(1H-pyrazolo[3,4-b]pyridin- |
| | 4-yl)-6,7-dihydro-4H-pyrazolo[5,1-c][1,4]oxazine; |
| 118 | (6*R,7*R)-2-(5-Fluoropyridin-2-yl)-6,7-dimethyl-3-(1H-pyrazolo[3,4- |
| | b]pyridin-4-yl)-6,7-dihydro-4H-pyrazolo[5,1-c][1,4]oxazine; |
| 119 | (6*S,7*S)-2-(5-Fluoropyridin-2-yl)-6,7-dimethyl-3-(1H-pyrazolo[3,4-b]pyridin- |
| | 4-yl)-6,7-dihydro-4H-pyrazolo[5,1-c][1,4]oxazine; |
| 120 | 2-(4-Fluorophenyl)-7,7-dimethyl-3-(1H-pyrazolo[3,4-b]pyridin-4-yl)-4,6- |
| | dihydropyrazolo[5,1-c][1,4]oxazine; |
| 121 | 2-(4-Fluorophenyl)-7,7-dimethyl-3-(6-methyl-1H-pyrazolo[3,4-b]pyridin-4-yl)- |
| | 4,6-dihydropyrazolo[5,1-c][1,4]oxazine; |
| 122 | 2-(5-Fluoro-2-pyridyl)-7,7-dimethyl-3-(1H-pyrazolo[3,4-b]pyridin-4-yl)-4,6- |
| | dihydropyrazolo[5,1-c][1,4]oxazine; |
| 123 | 2-(5-Fluoro-2-pyridyl)-7,7-dimethyl-3-(6-methyl-1H-pyrazolo[3,4-b]pyridin-4- |
| | yl)-4,6-dihydropyrazolo[5,1-c][1,4]oxazine; |
| 124 | 6,6-Dimethyl-3-(1H-pyrazolo[3,4-b]pyridin-4-yl)-2-(thiazol-4-yl)-6,7-dihydro- |
| | 4H-pyrazolo[5,1-c][1,4]oxazine; |
| 125 | 6,6-Dimethyl-3-(6-methyl-1H-pyrazolo[3,4-b]pyridin-4-yl)-2-(thiazol-4-yl)-6,7- |
| | dihydro-4H-pyrazolo[5,1-c][1,4]oxazine; |
| 126 | 6,6-Dimethyl-2-(oxazol-5-yl)-3-(1H-pyrazolo[3,4-b]pyridin-4-yl)-6,7-dihydro- |
| | 4H-pyrazolo[5,1-c][1,4]oxazine; |
| 127 | 6,6-Dimethyl-2-(1-methyl-1H-imidazol-4-yl)-3-(1H-pyrazolo[3,4-b]pyridin-4- |
| | yl)-6,7-dihydro-4H-pyrazolo[5,1-c][1,4]oxazine; |
| 128 | 2-(5-Fluoropyridin-2-yl)-6,6-dimethyl-3-(pyridin-4-yl)-6,7-dihydro-4H- |
| | pyrazolo[5,1-c][1,4]oxazin; |
| 129 | 3-(2-(Difluoromethyl)pyridin-4-yl)-2-(5-fluoropyridin-2-yl)-6,6-dimethyl-6,7- |
| | dihydro-4H-pyrazolo[5,1-c][1,4]oxazine; |
| 130 | 2-(5-Fluoropyridin-2-yl)-3-(3-methoxypyridin-4-yl)-6,6-dimethyl-6,7-dihydro- |
| | 4H-pyrazolo[5,1-c][1,4]oxazine; |
| 131 | N-(4-(2-(5-Fluoropyridin-2-yl)-6,6-dimethyl-6,7-dihydro-4H-pyrazolo[5,1- |
| | c][1,4]oxazin-3-yl)pyridin-2-yl)propionamide; |
| 132 | N-(4-(2-(5-Fluoropyridin-2-yl)-6,6-dimethyl-6,7-dihydro-4H-pyrazolo[5,1- |
| | c][1,4]oxazin-3-yl)pyridin-2-yl)isobutyramide; |
| 133 | 3,3,3-Trifluoro-N-(4-(2-(5-fluoropyridin-2-yl)-6,6-dimethyl-6,7-dihydro-4H- |
| | pyrazolo[5,1-c][1,4]oxazin-3-yl)pyridin-2-yl)propenamide; |
| 134 | N-(4-(2-(5-Fluoropyridin-2-yl)-6,6-dimethyl-6,7-dihydro-4H-pyrazolo[5,1- |
| | c][1,4]oxazin-3-yl)pyridin-2-yl)cyclopropanecarboxamide; |
| 135 | 2,2-Difluoro-N-(4-(2-(5-fluoropyridin-2-yl)-6,6-dimethyl-6,7-dihydro-4H- |
| | pyrazolo[5,1-c][1,4]oxazin-3-yl)pyridin-2-yl)cyclopropane-1-carboxamide; |
| 136 | N-(4-(2-(5-Fluoropyridin-2-yl)-6,6-dimethyl-6,7-dihydro-4H-pyrazolo[5,1- |
| | c][1,4]oxazin-3-yl)pyridin-2-yl)benzamide; |
| 137 | 2-(5-Fluoropyridin-2-yl)-6,6-dimethyl-3-(thieno[3,2-b]pyridin-7-yl)-6,7- |
| | dihydro-4H-pyrazolo[5,1-c][1,4]oxazine; |
| 138 | 2-(5-Fluoropyridin-2-yl)-6,6-dimethyl-3-(2-methyl-2H-pyrazolo[4,3-b]pyridin- |
| | 7-yl)-6,7-dihydro-4H-pyrazolo[5,1-c][1,4]oxazine; |
| 139 | 2-(5-Fluoropyridin-2-yl)-6,6-dimethyl-3-(1H-pyrazolo[4,3-b]pyridin-7-yl)-6,7- |
| | dihydro-4H-pyrazolo[5,1-c][1,4]oxazine; |
| 140 | 2-(5-Fluoropyridin-2-yl)-6,6-dimethyl-3-(pyrazolo[1,5-a]pyridin-5-yl)-6,7- |
| | dihydro-4H-pyrazolo[5,1-c][1,4]oxazine; |
| 141 | 3-(1-Ethyl-1H-pyrazolo[3,4-b]pyridin-5-yl)-2-(5-fluoropyridin-2-yl)-6,6- |
| | dimethyl-6,7-dihydro-4H-pyrazolo[5,1-c][1,4]oxazine; |
| 142 | 2-(5-Fluoropyridin-2-yl)-6,6-bis(methyl-d₃)-3-(1H-pyrazolo[3,4-b]pyridin-4- |
| | yl)-6,7-dihydro-4H-pyrazolo[5,1-c][1,4]oxazine; |
| 143 | 2-(5-Fluoropyridin-2-yl)-6,6-dimethyl-3-(1H-pyrazolo[3,4-b]pyridin-4-yl)-6,7- |
| | dihydro-4H-pyrazolo[5,1-c][1,4]oxazine-7,7-d₂; |
| 144 | 2-(4-Fluorophenyl)-6,6-dimethyl-3-(1H-pyrazolo[3,4-b]pyridin-4-yl)-6,7- |
| | dihydro-4H-pyrazolo[5,1-c][1,4]oxazine; |
| 145 | 2-(4-Fluorophenyl)-6,6-dimethyl-3-(6-methyl-1H-pyrazolo[3,4-b]pyridin-4-yl)- |
| | 6,7-dihydro-4H-pyrazolo[5,1-c][1,4]oxazine; |
| 146 | 2-(5-Chloropyridin-2-yl)-6,6-dimethyl-3-(1H-pyrazolo[3,4-b]pyridin-4-yl)-6,7- |
| | dihydro-4H-pyrazolo[5,1-c][1,4]oxazine; |
| 147 | 2-(5-Fluoropyridin-2-yl)-6,6-dimethyl-3-(1H-pyrrolo[2,3-b]pyridin-4-yl)-6,7- |
| | dihydro-4H-pyrazolo[5,1-c][1,4]oxazine; |
| 148 | 4-(2-(5-Fluoropyridin-2-yl)-6,6-dimethyl-6,7-dihydro-4H-pyrazolo[5,1- |
| | c][1,4]oxazin-3-yl)-1H-pyrrolo[2,3-b]pyridine-3-carbonitrile; |
| 149 | 2-(5-Fluoro-2-pyridyl)-6,6-dimethyl-3-(1H-pyrrolo[3,2-b]pyridin-7-yl)-4,7- |
| | dihydropyrazolo[5,1-c][1,4]oxazine; |
| 150 | 2-(5-Fluoropyridin-2-yl)-6,6-dimethyl-3-(1H-pyrazolo[3,4-b]pyridin-4-yl)-6,7- |
| | dihydro-4H-pyrazolo[5,1-c][1,4]oxazine; |
| 151 | 2-(5-Fluoropyridin-2-yl)-6,6-dimethyl-3-(1H-pyrazolo[3,4-b]pyridin-4-yl)-6,7- |
| | dihydro-4H-pyrazolo[5,1-c][1,4]oxazine-4,4-d₂; |
| 152 | 3-(3-Bromo-1H-pyrazolo[3,4-b]pyridin-4-yl)-2-(5-fluoropyridin-2-yl)-6,6- |
| | dimethyl-6,7-dihydro-4H-pyrazolo[5,1-c][1,4]oxazine; |
| 153 | 2-(5-Fluoro-2-pyridyl)-6,6-dimethyl-3-(1-methylpyrazolo[3,4-b]pyridin-4-yl)- |
| | 4,7-dihydropyrazolo[5,1-c][1,4]oxazine; |
| 154 | 2-(5-Fluoro-2-pyridyl)-6,6-dimethyl-3-(6-methyl-1H-pyrazolo[3,4-b]pyridin-4- |
| | yl)-4,7-dihydropyrazolo[5,1-c][1,4]oxazine; |
| 155 | 3-(6-Cyclopropyl-1H-pyrazolo[3,4-b]pyridin-4-yl)-2-(5-fluoropyridin-2-yl)-6,6- |
| | dimethyl-6,7-dihydro-4H-pyrazolo[5,1-c][1,4]oxazine; |
| 156 | 3-(3,6-Dimethyl-1H-pyrazolo[3,4-b]pyridin-4-yl)-2-(5-fluoro-2-pyridyl)-6,6- |
| | dimethyl-4,7-dihydropyrazolo[5,1-c][1,4]oxazine; |
| 157 | 3-(5-Fluoro-6-methyl-1H-pyrazolo[3,4-b]pyridin-4-yl)-2-(5-fluoro-2-pyridy])- |
| | 6,6-dimethyl-4,7-dihydropyrazolo[5,1-c][1,4]oxazine; |
| 158 | 2-(5-Fluoro-2-pyridyl)-6,6-dimethyl-3-(2-methylpyrazolo[3,4-b]pyridin-4-yl)- |
| | 4,7-dihydropyrazolo[5,1-c][1,4]oxazine; |
| 159 | 2-(5-Fluoropyridin-2-yl)-6,6-dimethyl-3-(1-methyl-1H-pyrazolo[4,3-b]pyridin- |
| | 7-yl)-6,7-dihydro-4H-pyrazolo[5,1-c][1,4]oxazine; |
| 160 | 2-(5-Fluoropyridin-2-yl)-6,6-dimethyl-3-(3-methyl-1H-pyrazolo[3,4-b]pyridin- |
| | 4-yl)-6,7-dihydro-4H-pyrazolo[5,1-c][1,4]oxazine; |
| 161 | 2-(6-Methoxypyridin-2-yl)-6,6-dimethyl-3-(1H-pyrazolo[3,4-b]pyridin-4-yl)- |
| | 6,7-dihydro-4H-pyrazolo[5,1-c][1,4]oxazine; |
| 162 | 2-(3-Chloropyridin-4-yl)-6,6-dimethyl-3-(1H-pyrazolo[3,4-b]pyridin-4-yl)-6,7- |
| | dihydro-4H-pyrazolo[5,1-c][1,4]oxazine; |
| 163 | 2-(5-Chloro-6-methylpyridin-2-yl)-6,6-dimethyl-3-(1H-pyrazolo[3,4-b]pyridin- |
| | 4-yl)-6,7-dihydro-4H-pyrazolo[5,1-c][1,4]oxazine; |
| 164 | 2-(5-Fluoro-6-methylpyridin-2-yl)-6,6-dimethyl-3-(1H-pyrazolo[3,4-b]pyridin- |
| | 4-yl)-6,7-dihydro-4H-pyrazolo[5,1-c][1,4]oxazine; |
| 165 | 2-(3,5-Difluoropyridin-4-yl)-6,6-dimethyl-3-(1H-pyrazolo[3,4-b]pyridin-4-yl)- |
| | 6,7-dihydro-4H-pyrazolo[5,1-c][1,4]oxazine; |
| 166 | 2-(3,5-Difluoropyridin-2-yl)-6,6-dimethyl-3-(1H-pyrazolo[3,4-b]pyridin-4-yl)- |
| | 6,7-dihydro-4H-pyrazolo[5,1-c][1,4]oxazine; |
| 167 | 2-(3,5-Difluoropyridin-2-yl)-6,6-dimethyl-3-(6-methyl-1H-pyrazolo[3,4- |
| | b]pyridin-4-yl)-6,7-dihydro-4H-pyrazolo[5,1-c][1,4]oxazine; |
| 168 | 2-(5-Fluoropyridin-2-yl)-3-(6-methoxy-1,5-naphthyridin-4-yl)-6,6-dimethyl- |
| | 6,7-dihydro-4H-pyrazolo[5,1-c][1,4]oxazine; |
| 169 | (*R)-6-Ethyl-2-(5-fluoropyridin-2-yl)-6-methyl-3-(1H-pyrazolo[3,4-b]pyridin- |
| | 4-yl)-6,7-dihydro-4H-pyrazolo[5,1-c][1,4]oxazine; |
| 170 | (*S)-6-Ethyl-2-(5-fluoropyridin-2-yl)-6-methyl-3-(1H-pyrazolo[3,4-b]pyridin- |
| | 4-yl)-6,7-dihydro-4H-pyrazolo[5,1-c][1,4]oxazine; |
| 171 | (*S)-2-(4-Fluorophenyl)-6,6,7-trimethyl-3-(1H-pyrazolo[3,4-b]pyridin-4-yl)- |
| | 6,7-dihydro-5H-pyrazolo[5,1-b][1,3]oxazine; |
| 172 | (*R)-2-(4-Fluorophenyl)-6,6,7-trimethyl-3-(1H-pyrazolo[3,4-b]pyridin-4-yl)- |
| | 6,7-dihydro-5H-pyrazolo[5,1-b][1,3]oxazine; |
| 173 | (*R)-2-(5-Fluoropyridin-2-yl)-6,6,7-trimethyl-3-(1H-pyrazolo[3,4-b]pyridin-4- |
| | yl)-6,7-dihydro-4H-pyrazolo[5,1-c][1,4]oxazine; |
| 174 | (*S)-2-(5-Fluoropyridin-2-yl)-6,6,7-trimethyl-3-(1H-pyrazolo[3,4-b]pyridin-4- |
| | yl)-6,7-dihydro-4H-pyrazolo[5,1-c][1,4]oxazine; |
| 175 | 4-(2-(5-Fluoropyridin-2-yl)-6-methyl-6-(trifluoromethyl)-6,7-dihydro-4H- |
| | pyrazolo[5,1-c][1,4]oxazin-3-yl)-N-methylpicolinamide; |
| 176 | 2-(5-Fluoropyridin-2-yl)-6-methyl-3-(1H-pyrazolo[3,4-b]pyridin-4-yl)-6- |
| | (trifluoromethyl)-6,7-dihydro-4H-pyrazolo[5,1-c][1,4]oxazine; |
| 177 | (*S)-2-(5-Fluoropyridin-2-yl)-6-methyl-3-(1H-pyrazolo[3,4-b]pyridin-4-yl)-6- |
| | (trifluoromethyl)-6,7-dihydro-4H-pyrazolo[5,1-c][1,4]oxazine; |
| 178 | (*R)-2-(5-Fluoropyridin-2-yl)-6-methyl-3-(1H-pyrazolo[3,4-b]pyridin-4-yl)-6- |
| | (trifluoromethyl)-6,7-dihydro-4H-pyrazolo[5,1-c][1,4]oxazine; |
| 179 | (*S)-2-(5-Fluoropyridin-2-yl)-6-methyl-3-(6-methyl-1H-pyrazolo[3,4- |
| | b]pyridin-4-yl)-6-(trifluoromethyl)-6,7-dihydro-4H-pyrazolo[5,1- |
| | c][1,4]oxazine; |
| 180 | (*R)-2-(5-Fluoropyridin-2-yl)-6-methyl-3-(6-methyl-1H-pyrazolo[3,4- |
| | b]pyridin-4-yl)-6-(trifluoromethyl)-6,7-dihydro-4H-pyrazolo[5,1- |
| | c][1,4]oxazine; |
| 181 | 2-(4-Fluorophenyl)-3-(1H-pyrazolo[3,4-b]pyridin-4-yl)-6,7- |
| | dihydropyrazolo[5,1-c][1,4]oxazin-4-one; |
| 182 | 2-(5-Fluoro-2-pyridyl)-3-(1H-pyrazolo[3,4-b]pyridin-4-yl)-6,7- |
| | dihydropyrazolo[5,1-c][1,4]oxazin-4-one; |
| 183 | N-(4-(2-(5-Fluoropyridin-2-yl)-6,7-dihydro-5H-pyrazolo[5,1-b][1,3]oxazin-3- |
| | yl)pyridin-2-yl)acetamide; |
| 184 | 2-(4-Fluorophenyl)-3-(1H-pyrazolo[3,4-b]pyridin-4-yl)-6,7-dihydro-5H- |
| | pyrazolo[5,1-b][1,3]oxazine; |
| 185 | 2-(4-Fluorophenyl)-3-(6-methyl-1H-pyrazolo[3,4-b]pyridin-4-yl)-6,7-dihydro- |
| | 5H-pyrazolo[5,1-b][1,3]oxazine; |
| 186 | 2-(5-Fluoropyridin-2-yl)-3-(1H-pyrazolo[3,4-b]pyridin-4-yl)-6,7-dihydro-5H- |
| | pyrazolo[5,1-b][1,3]oxazine; |
| 187 | 2-(5-Fluoropyridin-2-yl)-3-(6-methyl-1H-pyrazolo[3,4-b]pyridin-4-yl)-6,7- |
| | dihydro-5H-pyrazolo[5,1-b][1,3]oxazine; |
| 188 | 2-(3,5-Difluoro-2-pyridyl)-3-(1H-pyrazolo[3,4-b]pyridin-4-yl)-6,7-dihydro-5H- |
| | pyrazolo[5,1-b][1,3]oxazine; |
| 189 | 2-(3,5-Difluoro-2-pyridyl)-3-(6-methyl-1H-pyrazolo[3,4-b]pyridin-4-yl)-6,7- |
| | dihydro-5H-pyrazolo[5,1-b][1,3]oxazine; |
| 190 | 2-(5-Fluoro-2-pyridyl)-3-(6-methoxy-1,5-naphthyridin-4-yl)-6,7-dihydro-5H- |
| | pyrazolo[5,1-b][1,3]oxazine; |
| 191 | (R/S)-2-(4-Fluorophenyl)-6-methyl-3-(1H-pyrazolo[3,4-b]pyridin-4-yl)-6,7- |
| | dihydro-5H-pyrazolo[5,1-b][1,3]oxazine; |
| 192 | (R/S)-2-(4-Fluorophenyl)-6-methyl-3-(6-methyl-1H-pyrazolo[3,4-b]pyridin-4- |
| | yl)-6,7-dihydro-5H-pyrazolo[5,1-b][1,3]oxazine; |
| 193 | (*S)-2-(4-Fluorophenyl)-6-methyl-3-(6-methyl-1H-pyrazolo[3,4-b]pyridin-4- |
| | yl)-6,7-dihydro-5H-pyrazolo[5,1-b][1,3]oxazine; |
| 194 | (*R)-2-(4-Fluorophenyl)-6-methyl-3-(6-methyl-1H-pyrazolo[3,4-b]pyridin-4- |
| | yl)-6,7-dihydro-5H-pyrazolo[5,1-b][1,3]oxazine; |
| 195 | 2-(5-Fluoropyridin-2-yl)-6-methyl-3-(thieno[3,2-b]pyridin-7-yl)-6,7-dihydro- |
| | 5H-pyrazolo[5,1-b][1,3]oxazine; |
| 196 | 2-(5-Fluoropyridin-2-yl)-6-methyl-3-(1H-pyrazolo[3,4-b]pyridin-4-yl)-6,7- |
| | dihydro-5H-pyrazolo[5,1-b][1,3]oxazine; |
| 197 | 2-(5-Fluoropyridin-2-yl)-6-methyl-3-(6-methyl-1H-pyrazolo[3,4-b]pyridin-4- |
| | yl)-6,7-dihydro-5H-pyrazolo[5,1-b][1,3]oxazine; |
| 198 | (*S)-2-(5-Fluoro-2-pyridyl)-5-methyl-3-(1H-pyrazolo[3,4-b]pyridin-4-yl)-6,7- |
| | dihydro-5H-pyrazolo[5,1-b][1,3]oxazine; |
| 199 | (*R)-2-(5-Fluoro-2-pyridyl)-5-methyl-3-(1H-pyrazolo[3,4-b]pyridin-4-yl)-6,7- |
| | dihydro-5H-pyrazolo[5,1-b][1,3]oxazine; |
| 200 | (*S)-2-(5-Fluoro-2-pyridyl)-5-methyl-3-(6-methyl-1H-pyrazolo[3,4-b]pyridin- |
| | 4-yl)-6,7-dihydro-5H-pyrazolo[5,1-b][1,3]oxazine; |
| 201 | (*R)-2-(5-Fluoro-2-pyridyl)-5-methyl-3-(6-methyl-1H-pyrazolo[3,4-b]pyridin- |
| | 4-yl)-6,7-dihydro-5H-pyrazolo[5,1-b][1,3]oxazine; |
| 202 | (5*S,7*R)-2-(5-Fluoro-2-pyridyl)-5,7-dimethyl-3-(1H-pyrazolo[3,4-b]pyridin- |
| | 4-yl)-6,7-dihydro-5H-pyrazolo[5,1-b][1,3]oxazine; |
| 203 | (5*R,7*S)-2-(5-Fluoro-2-pyridyl)-5,7-dimethyl-3-(1H-pyrazolo[3,4-b]pyridin- |
| | 4-yl)-6,7-dihydro-5H-pyrazolo[5,1-b][1,3]oxazine; |
| 204 | (5*R,7*R)-2-(5-Fluoropyridin-2-yl)-5,7-dimethyl-3-(1H-pyrazolo[3,4- |
| | b]pyridin-4-yl)-6,7-dihydro-5H-pyrazolo[5,1-b][1,3]oxazine; |
| 205 | (5*S,7*S)-2-(5-Fluoropyridin-2-yl)-5,7-dimethyl-3-(1H-pyrazolo[3,4-b]pyridin- |
| | 4-yl)-6,7-dihydro-5H-pyrazolo[5,1-b][1,3]oxazine; |
| 206 | (5*S,7*R)-2-(5-Fluoro-2-pyridyl)-5,7-dimethyl-3-(6-methyl-1H-pyrazolo[3,4- |
| | b]pyridin-4-yl)-6,7-dihydro-5H-pyrazolo[5,1-b][1,3]oxazine; |
| 207 | (5*R,7*S)-2-(5-Fluoro-2-pyridyl)-5,7-dimethyl-3-(6-methyl-1H-pyrazolo[3,4- |
| | b]pyridin-4-yl)-6,7-dihydro-5H-pyrazolo[5,1-b][1,3]oxazine; |
| 208 | (5*R,7*R)-2-(5-Fluoro-2-pyridyl)-5,7-dimethyl-3-(6-methyl-1H-pyrazolo[3,4- |
| | b]pyridin-4-yl)-6,7-dihydro-5H-pyrazolo[5,1-b][1,3]oxazine; |
| 209 | (5*S,7*S)-2-(5-Fluoro-2-pyridyl)-5,7-dimethyl-3-(6-methyl-1H-pyrazolo[3,4- |
| | b]pyridin-4-yl)-6,7-dihydro-5H-pyrazolo[5,1-b][1,3]oxazine; |
| 210 | 2-(4-Fluorophenyl)-3-(1H-pyrazolo[3,4-b]pyridin-4-yl)spiro[5,7- |
| | dihydropyrazolo[5,1-b][1,3]oxazine-6,3′-oxetane]; |
| 211 | 2′-(4-Fluorophenyl)-3′-(6-methyl-1H-pyrazolo[3,4-b]pyridin-4-yl)-5′H,7′H- |
| | spiro[oxetane-3,6′-pyrazolo[5,1-b][1,3]oxazine]; |
| 212 | 2-(4-Fluorophenyl)-6,6-dimethyl-3-(1H-pyrazolo[3,4-b]pyridin-4-yl)-6,7- |
| | dihydro-5H-pyrazolo[5,1-b][1,3]oxazine; |
| 213 | 2-(4-Fluorophenyl)-6,6-dimethyl-3-(6-methyl-1H-pyrazolo[3,4-b]pyridin-4-yl)- |
| | 6,7-dihydro-5H-pyrazolo[5,1-b][1,3]oxazine; |
| 214 | 2-(5-Fluoropyridin-2-yl)-6,6-dimethyl-3-(thieno[3,2-b]pyridin-7-yl)-6,7- |
| | dihydro-5H-pyrazolo[5,1-b][1,3]oxazine; |
| 215 | N-(4-(2-(4-Fluorophenyl)-5,6,7,8-tetrahydropyrazolo[5,1-b][1,3]oxazepin-3- |
| | yl)pyridin-2-yl)acetamide; |
| 216 | 2-(4-Fluorophenyl)-3-(1H-pyrazolo[3,4-b]pyridin-4-yl)-5,6,7,8- |
| | tetrahydropyrazolo[5,1-b][1,3]oxazepane; |
| 217 | 2-(4-Fluorophenyl)-3-(6-methyl-1H-pyrazolo[3,4-b]pyridin-4-yl)-5,6,7,8- |
| | tetrahydropyrazolo[5,1-b][1,3]oxazepane; |
| 218 | 2-(5-Fluoro-2-pyridyl)-3-(1H-pyrazolo[3,4-b]pyridin-4-yl)-5,6,7,8- |
| | tetrahydropyrazolo[5,1-b][1,3]oxazepane; |
| 219 | 2-(5-Fluoro-2-pyridyl)-3-(6-methyl-1H-pyrazolo[3,4-b]pyridin-4-yl)-5,6,7,8- |
| | tetrahydropyrazolo[5,1-b][1,3]oxazepane; |
| 220 | 7-[2-(5-Fluoro-2-pyridyl)-5,6-dihydro-4H-pyrrolo[1,2-b]pyrazol-3-yl]-1H- |
| | pyrazolo[4,3-b]pyridine; |
| 221 | 5-Fluoro-4-[2-(5-fluoro-2-pyridyl)-5,6-dihydro-4H-pyrrolo[1,2-b]pyrazol-3-yl]- |
| | 1H-pyrazolo[3,4-b]pyridine; |
| 222 | 6-(Difluoromethyl)-4-[2-(5-fluoro-2-pyridyl)-5,6-dihydro-4H-pyrrolo[1,2- |
| | b]pyrazol-3-yl]-1H-pyrazolo[3,4-b]pyridine; |
| 223 | 1-Ethyl-5-(2-(5-fluoropyridin-2-yl)-5,6-dihydro-4H-pyrrolo[1,2-b]pyrazol-3- |
| | yl)-1H-pyrazolo[3,4-b]pyridine; |
| 224 | 3-Chloro-5-fluoro-4-(2-(5-fluoropyridin-2-yl)-5,6-dihydro-4H-pyrrolo[1,2- |
| | b]pyrazol-3-yl)-6-methyl-1H-pyrazolo[3,4-b]pyridine; |
| 225 | 4-[2-(4-Fluorophenyl)-5,6-dihydro-4H-pyrrolo[1,2-b]pyrazol-3-yl]-6-methyl- |
| | 1H-pyrazolo[3,4-d]pyrimidine; |
| 226 | 5-[(5S)-5-Fluoro-2-(4-fluorophenyl)-5,6-dihydro-4H-pyrrolo[1,2-b]pyrazol-3- |
| | yl]pyrazolo[1,5-a]pyridine; |
| 227 | 5-Fluoro-4-[(5S)-5-fluoro-2-(4-fluorophenyl)-5,6-dihydro-4H-pyrrolo[1,2- |
| | b]pyrazol-3-yl]-1H-pyrazolo[3,4-b]pyridine; |
| 228 | 4-[(5R)-5-Fluoro-2-(4-fluorophenyl)-5,6-dihydro-4H-pyrrolo[1,2-b]pyrazol-3- |
| | yl]-1H-pyrazolo[3,4-b]pyridine; |
| 229 | N-[4-[(5S)-5-Fluoro-2-(4-fluorophenyl)-5,6-dihydro-4H-pyrrolo[1,2-b]pyrazol- |
| | 3-yl]-2-pyridyl]acetamide; |
| 230 | 4-[5,5-Difluoro-2-(4-fluorophenyl)-4,6-dihydropyrrolo[1,2-b]pyrazol-3-yl]-1H- |
| | pyrazolo[3,4-b]pyridine; |
| 231 | 1H-pyrazolo[3,4-b]pyridine; |
| 232 | 4-[2-(4-Fluorophenyl)-5,5-dimethyl-4,6-dihydropyrrolo[1,2-b]pyrazol-3-yl]-6- |
| | methyl-1H-pyrazolo[3,4-b]pyridine; |
| 233 | 1H-pyrrolo[2,3-b]pyridi; |
| 234 | 4-[2-(5-Fluoro-2-pyridyl)-5,5-dimethyl-4,6-dihydropyrrolo[1,2-b]pyrazol-3-yl]- |
| | 1H-pyrazolo[3,4-b]pyridine; |
| 235 | 4-[2-(5-Fluoro-2-pyridyl)-5,5-dimethyl-4,6-dihydropyrrolo[1,2-b]pyrazol-3-yl]- |
| | 6-methyl-1H-pyrrolo[2,3-b]pyridine; |
| 236 | 5-Fluoro-4-[2-(5-fluoro-2-pyridyl)-5,5-dimethyl-4,6-dihydropyrrolo[1,2- |
| | b]pyrazol-3-yl]-1H-pyrazolo[3,4-b]pyridine; |
| 237 | 3-Chloro-4-(2-(5-fluoropyridin-2-yl)-5,5-dimethyl-5,6-dihydro-4H-pyrrolo[1,2- |
| | b]pyrazol-3-yl)-1H-pyrazolo[3,4-b]pyridine; |
| 238 | 4-[2-(5-Fluoro-2-pyridyl)-5,5-dimethyl-4,6-dihydropyrrolo[1,2-b]pyrazol-3-yl]- |
| | 6-methyl-1H-pyrazolo[3,4-b]pyridine; |
| 239 | 6-(Difluoromethyl)-4-[2-(5-fluoro-2-pyridyl)-5,5-dimethyl-4,6- |
| | dihydropyrrolo[1,2-b]pyrazol-3-yl]-1H-pyrazolo[3,4-b]pyridine; |
| 240 | 5-Fluoro-4-[2-(5-fluoro-2-pyridyl)-5,5-dimethyl-4,6-dihydropyrrolo[1,2- |
| | b]pyrazol-3-yl]-6-methyl-1H-pyrazolo[3,4-b]pyridine; |
| 241 | 4-[2-(4-Fluorophenyl)-5,5-bis(methyl-d₃)-4,6-dihydropyrrolo[1,2-b]pyrazol-3- |
| | yl]-1H-pyrazolo[3,4-b]pyridine; |
| 242 | 4-[2-(4-Fluorophenyl)-5,5-bis(methyl-d₃)-4,6-dihydropyrrolo[1,2-b]pyrazol-3- |
| | yl]-6-methyl-1H-pyrazolo[3,4-b]pyridine; |
| 243 | 5-Fluoro-4-[2-(5-fluoro-2-pyridyl)-5,5-bis(methyl-d₃)-4,6-dihydropyrrolo[1,2- |
| | b]pyrazol-3-yl]-1H-pyrazolo[3,4-b]pyridine; |
| 244 | 5-Fluoro-4-[2-(5-fluoro-2-pyridyl)-5,5-bis(methyl-d₃)-4,6-dihydropyrrolo[1,2- |
| | b]pyrazol-3-yl]-6-methyl-1H-pyrazolo[3,4-b]pyridine; |
| 245 | 1H-pyrazolo[3,4-b]pyridine; |
| 246 | 4-[2-(4-Fluorophenyl)-4,4-dimethyl-5,6-dihydropyrrolo[1,2-b]pyrazol-3-yl]-6- |
| | methyl-1H-pyrazolo[3,4-b]pyridine; |
| 247 | 2-(4-Fluorophenyl)-3-(1H-pyrazolo[3,4-b]pyridin-4-yl)spiro[4,6- |
| | dihydropyrrolo[1,2-b]pyrazole-5,1′-cyclopropane]; |
| 248 | 2-(4-Fluorophenyl)-3-(6-methyl-1H-pyrazolo[3,4-b]pyridin-4-yl)spiro[4,6- |
| | dihydropyrrolo[1,2-b]pyrazole-5,1′-cyclopropane]; |
| 249 | 2-(5-Fluoro-2-pyridyl)-3-(1H-pyrazolo[3,4-b]pyridin-4-yl)spiro[4,5- |
| | dihydropyrrolo[1,2-b]pyrazole-6,1′-cyclopropane]; |
| 250 | (*S)-1′,1′-Difluoro-2-(4-fluorophenyl)-3-(1H-pyrazolo[3,4-b]pyridin-4- |
| | yl)spiro[4,6-dihydropyrrolo[1,2-b]pyrazole-5,2′-cyclopropane]; |
| 251 | (*R)-1′,1′-Difluoro-2-(4-fluorophenyl)-3-(1H-pyrazolo[3,4-b]pyridin-4- |
| | yl)spiro[4,6-dihydropyrrolo[1,2-b]pyrazole-5,2′-cyclopropane]; |
| 252 | (*S)-1′,1′-Difluoro-2-(4-fluorophenyl)-3-(6-methyl-1H-pyrazolo[3,4-b]pyridin- |
| | 4-yl)spiro[4,6-dihydropyrrolo[1,2-b]pyrazole-5,2′-cyclopropane]; |
| 253 | (*R)-1′,1′-Difluoro-2-(4-fluorophenyl)-3-(6-methyl-1H-pyrazolo[3,4-b]pyridin- |
| | 4-yl)spiro[4,6-dihydropyrrolo[1,2-b]pyrazole-5,2′-cyclopropane]; |
| 254 | (*S)-1′,1′-Difluoro-2-(5-fluoro-2-pyridyl)-3-(1H-pyrazolo[3,4-b]pyridin-4- |
| | yl)spiro[4,6-dihydropyrrolo[1,2-b]pyrazole-5,2′-cyclopropane]; |
| 255 | (*R)-1′,1′-Difluoro-2-(5-fluoro-2-pyridyl)-3-(1H-pyrazolo[3,4-b]pyridin-4- |
| | yl)spiro[4,6-dihydropyrrolo[1,2-b]pyrazole-5,2′-cyclopropane]; |
| 256 | (*S)-3-(3-Chloro-1H-pyrazolo[3,4-b]pyridin-4-yl)-1′,1′-difluoro-2-(5-fluoro-2- |
| | pyridyl)spiro[4,6-dihydropyrrolo[1,2-b]pyrazole-5,2′-cyclopropane]; |
| 257 | (*R)-3-(3-Chloro-1H-pyrazolo[3,4-b]pyridin-4-yl)-1′,1′-difluoro-2-(5-fluoro-2- |
| | pyridyl)spiro[4,6-dihydropyrrolo[1,2-b]pyrazole-5,2′-cyclopropane]; |
| 258 | (*S)-2,2-Difluoro-3′-(5-fluoro-1H-pyrazolo[3,4-b]pyridin-4-yl)-2′-(5- |
| | fluoropyridin-2-yl)-4′H,6′H-spiro[cyclopropane-1,5′-pyrrolo[1,2-b]pyrazole]; |
| 259 | (*R)-1′,1′-Difluoro-3-(5-fluoro-1H-pyrazolo[3,4-b]pyridin-4-yl)-2-(5-fluoro-2- |
| | pyridyl)spiro[4,6-dihydropyrrolo[1,2-b]pyrazole-5,2′-cyclopropane]; |
| 260 | (*S)-1′,1′-Difluoro-2-(5-fluoro-2-pyridyl)-3-(6-methyl-1H-pyrazolo[3,4- |
| | b]pyridin-4-yl)spiro[4,6-dihydropyrrolo[1,2-b]pyrazole-5,2′-cyclopropane]; |
| 261 | (*R)-1′,1′-Difluoro-2-(5-fluoro-2-pyridyl)-3-(6-methyl-1H-pyrazolo[3,4- |
| | b]pyridin-4-yl)spiro[4,6-dihydropyrrolo[1,2-b]pyrazole-5,2′-cyclopropane]; |
| 262 | (*S)-3′-(3-Chloro-6-methyl-1H-pyrazolo[3,4-b]pyridin-4-yl)-2,2-difluoro-2′-(5- |
| | fluoropyridin-2-yl)-4′H,6′H-spiro[cyclopropane-1,5′-pyrrolo[1,2-b]pyrazole]; |
| 263 | (*R)-1′,1′-Difluoro-3-(5-fluoro-6-methyl-1H-pyrazolo[3,4-b]pyridin-4-yl)-2-(5- |
| | fluoro-2-pyridyl)spiro[4,6-dihydropyrrolo[1,2-b]pyrazole-5,2′-cyclopropane]; |
| 264 | (*S)-1′,1′-Difluoro-3-(5-fluoro-6-methyl-1H-pyrazolo[3,4-b]pyridin-4-yl)-2-(5- |
| | fluoro-2-pyridyl)spiro[4,6-dihydropyrrolo[1,2-b]pyrazole-5,2′-cyclopropane]; |
| 265 | (*R)-1′,1′-Difluoro-3-(5-fluoro-3,6-dimethyl-1H-pyrazolo[3,4-b]pyridin-4-yl)- |
| | 2-(5-fluoro-2-pyridyl)spiro[4,6-dihydropyrrolo[1,2-b]pyrazole-5,2′- |
| | cyclopropane]; |
| 266 | (*S)-1′,1′-Difluoro-3-(5-fluoro-3,6-dimethyl-1H-pyrazolo[3,4-b]pyridin-4-yl)-2- |
| | (5-fluoro-2-pyridyl)spiro[4,6-dihydropyrrolo[1,2-b]pyrazole-5,2′- |
| | cyclopropane]; |
| 267 | (4aR,5aR)-2-(4-Fluorophenyl)-3-(1H-pyrazolo[3,4-b]pyridin-4-yl)-4,4a,5,5a- |
| | tetrahydrocyclopropa[4,5]pyrrolo[1,2-b]pyrazole; |
| 268 | (4aS,5aS)-2-(4-Fluorophenyl)-3-(1H-pyrazolo[3,4-b]pyridin-4-yl)-4,4a,5,5a- |
| | tetrahydrocyclopropa[4,5]pyrrolo[1,2-b]pyrazole; |
| 269 | (4aR,5aR)-2-(4-Fluorophenyl)-3-(6-methyl-1-((2- |
| | (trimethylsilyl)ethoxy)methyl)-1H-pyrazolo[3,4-b]pyridin-4-yl)-4,4a,5,5a- |
| | tetrahydrocyclopropa[4,5]pyrrolo[1,2-b]pyrazole; |
| 270 | (4aS,5aS)-2-(4-Fluorophenyl)-3-(6-methyl-1-((2-(trimethylsilyl)ethoxy)methyl)- |
| | 1H-pyrazolo[3,4-b]pyridin-4-yl)-4,4a,5,5a- |
| | tetrahydrocyclopropa[4,5]pyrrolo[1,2-b]pyrazole; |
| 271 | (4aS,5aS)-3-(3-Chloro-1H-pyrazolo[3,4-b]pyridin-4-yl)-2-(5-fluoropyridin-2- |
| | yl)-4,4a,5,5a-tetrahydrocyclopropa[4,5]pyrrolo[1,2-b]pyrazole; |
| 272 | (4aS,5aS)-3-(5-Fluoro-1H-pyrazolo[3,4-b]pyridin-4-yl)-2-(5-fluoropyridin-2- |
| | yl)-4,4a,5,5a-tetrahydrocyclopropa[4,5]pyrrolo[1,2-b]pyrazole; |
| 273 | (4aS,5aS)-3-(5-Fluoro-6-methyl-1H-pyrazolo[3,4-b]pyridin-4-y])-2-(5- |
| | fluoropyridin-2-yl)-4,4a,5,5a-tetrahydrocyclopropa[4,5]pyrrolo[1,2-b]pyrazole; |
| 274 | (4aS,5aS)-3-(5-Fluoro-3,6-dimethyl-1H-pyrazolo[3,4-b]pyridin-4-yl)-2-(5- |
| | fluoropyridin-2-yl)-4,4a,5,5a-tetrahydrocyclopropa[4,5]pyrrolo[1,2-b]pyrazole; |
| 275 | (Racemic) 2-(4-Fluorophenyl)-3-(1H-pyrazolo[3,4-b]pyridin-4-yl)-3b,4,4a,5- |
| | tetrahydrocyclopropa[3,4]pyrrolo[1,2-b]pyrazole; |
| 276 | (3b*R,4a*S)-2-(4-Fluorophenyl)-3-(6-methyl-1H-pyrazolo[3,4-b]pyridin-4-yl)- |
| | 3b,4,4a,5-tetrahydrocyclopropa[3,4]pyrrolo[1,2-b]pyrazole; |
| 277 | (3b*S,4a*R)-2-(4-Fluorophenyl)-3-(6-methyl-1H-pyrazolo[3,4-b]pyridin-4-yl)- |
| | 3b,4,4a,5-tetrahydrocyclopropa[3,4]pyrrolo[1,2-b]pyrazole; |
| 278 | 4-[5,5-Difluoro-2-(4-fluorophenyl)-6,7-dihydro-4H-pyrazolo[1,5-a]pyridin-3- |
| | yl]-5-fluoro-1H-pyrazolo[3,4-b]pyridine; |
| 279 | 4-[6,6-Difluoro-2-(4-fluorophenyl)-5,7-dihydro-4H-pyrazolo[1,5-a]pyridin-3- |
| | yl]-6-methyl-1H-pyrazolo[3,4-d]pyrimidine; |
| 280 | 4-(6,6-Difluoro-2-(4-fluorophenyl)-4,5,6,7-tetrahydropyrazolo[1,5-a]pyridin-3- |
| | yl)-5-fluoro-1H-pyrazolo[3,4-b]pyridine; |
| 281 | 4-[2-(5-Fluoro-2-pyridyl)-6,6-dimethyl-5,7-dihydro-4H-pyrazolo[1,5-a]pyridin- |
| | 3-yl]pyridin-2-amine; |
| 282 | 5-Fluoro-4-[2-(5-fluoro-2-pyridyl)-6,6-dimethyl-5,7-dihydro-4H-pyrazolo[1,5- |
| | a]pyridin-3-yl]-1H-pyrazolo[3,4-b]pyridine; |
| 283 | 4-[2-(5-Fluoro-2-pyridyl)-6,6-bis(methyl-d₃)-5,7-dihydro-4H-pyrazolo[1,5- |
| | alpyridin-3-yl]-1H-pyrazolo[3,4-b]pyridine; |
| 284 | (*S)-6-(Fluoromethyl)-2-(5-fluoro-2-pyridyl)-6-methyl-3-(4-pyridyl)-5,7- |
| | dihydro-4H-pyrazolo[1,5-a]pyridine; |
| 285 | (*R)-6-(Fluoromethyl)-2-(5-fluoro-2-pyridyl)-6-methyl-3-(4-pyridyl)-5,7- |
| | dihydro-4H-pyrazolo[1,5-a]pyridine; |
| 286 | (*S)-4-[6-(Fluoromethyl)-2-(5-fluoro-2-pyridyl)-6-methyl-5,7-dihydro-4H- |
| | pyrazolo[1,5-a]pyridin-3-yl]pyridin-2-amine; |
| 287 | (*R)-4-[6-(Fluoromethyl)-2-(5-fluoro-2-pyridyl)-6-methyl-5,7-dihydro-4H- |
| | pyrazolo[1,5-a]pyridin-3-yl]pyridin-2-amine; |
| 288 | (Racemic) 3-(2,5-Difluoro-4-pyridyl)-6-(fluoromethyl)-2-(5-fluoro-2-pyridyl)- |
| | 6-methyl-5,7-dihydro-4H-pyrazolo[1,5-a]pyridine; |
| 289 | (*R)-5-[6-(Fluoromethyl)-2-(5-fluoro-2-pyridyl)-6-methyl-5,7-dihydro-4H- |
| | pyrazolo[1,5-a]pyridin-3-yl]pyrazolo[1,5-a]pyridine; |
| 290 | (*S)-5-[6-(Fluoromethyl)-2-(5-fluoro-2-pyridyl)-6-methyl-5,7-dihydro-4H- |
| | pyrazolo[1,5-a]pyridin-3-yl]pyrazolo[1,5-a]pyridine; |
| 291 | (*R)-3-Chloro-4-(6-(fluoromethyl)-2-(5-fluoropyridin-2-yl)-6-methyl-4,5,6,7- |
| | tetrahydropyrazolo[1,5-a]pyridin-3-yl)-1H-pyrazolo[3,4-b]pyridine; |
| 292 | (*R)-5-Fluoro-4-[6-(fluoromethyl)-2-(5-fluoro-2-pyridy])-6-methyl-5,7- |
| | dihydro-4H-pyrazolo[1,5-a]pyridin-3-yl]-1H-pyrazolo[3,4-b]pyridine; |
| 293 | (*S)-5-Fluoro-4-[6-(fluoromethyl)-2-(5-fluoro-2-pyridyl)-6-methyl-5,7-dihydro- |
| | 4H-pyrazolo[1,5-a]pyridin-3-yl]-1H-pyrazolo[3,4-b]pyridine; |
| 294 | (*R)-5-Fluoro-4-(6-(fluoromethyl-d₂)-2-(5-fluoropyridin-2-yl)-6-(methyl-d₃)- |
| | 4,5,6,7-tetrahydropyrazolo[1,5-a]pyridin-3-yl)-1H-pyrazolo[3,4-b]pyridine; |
| 295 | (*S)-5-Fluoro-4-(6-(fluoromethyl-d₂)-2-(5-fluoropyridin-2-yl)-6-(methyl-d₃)- |
| | 4,5,6,7-tetrahydropyrazolo[1,5-a]pyridin-3-yl)-1H-pyrazolo[3,4-b]pyridine; |
| 296 | (*S)-4-[2-(5-Fluoro-2-pyridyl)-6-(methoxymethyl)-4,5,6,7- |
| | tetrahydropyrazolo[1,5-a]pyridin-3-yl]-6-methyl-1H-pyrazolo[3,4-b]pyridine; |
| 297 | (*R)-4-[2-(5-Fluoro-2-pyridyl)-6-(methoxymethyl)-4,5,6,7- |
| | tetrahydropyrazolo[1,5-a]pyridin-3-yl]-6-methyl-1H-pyrazolo[3,4-b]pyridine; |
| 298 | (*S)-4-[6-Fluoro-2-(5-fluoro-2-pyridyl)-6-(methoxymethyl)-5,7-dihydro-4H- |
| | pyrazolo[1,5-a]pyridin-3-yl]-6-methyl-1H-pyrrolo[2,3-b]pyridine; |
| 299 | (*R)-4-[6-Fluoro-2-(5-fluoro-2-pyridyl)-6-(methoxymethyl)-5,7-dihydro-4H- |
| | pyrazolo[1,5-a]pyridin-3-yl]-6-methyl-1H-pyrrolo[2,3-b]pyridine; |
| 300 | (*S)-4-[6-Fluoro-2-(5-fluoro-2-pyridy])-6-(methoxymethyl)-5,7-dihydro-4H- |
| | pyrazolo[1,5-a]pyridin-3-yl]-1H-pyrazolo[3,4-b]pyridine; |
| 301 | (*R)-4-[6-Fluoro-2-(5-fluoro-2-pyridyl)-6-(methoxymethyl)-5,7-dihydro-4H- |
| | pyrazolo[1,5-a]pyridin-3-yl]-1H-pyrazolo[3,4-b]pyridine; |
| 302 | (*S)-5-Fluoro-4-[6-fluoro-2-(5-fluoro-2-pyridyl)-6-(methoxymethyl)-5,7- |
| | dihydro-4H-pyrazolo[1,5-a]pyridin-3-yl]-1H-pyrazolo[3,4-b]pyridine; |
| 303 | (*R)-5-Fluoro-4-[6-fluoro-2-(5-fluoro-2-pyridyl)-6-(methoxymethyl)-5,7- |
| | dihydro-4H-pyrazolo[1,5-a]pyridin-3-yl]-1H-pyrazolo[3,4-b]pyridine; |
| 304 | (*S)-4-[6-Fluoro-2-(5-fluoro-2-pyridyl)-6-(methoxymethyl)-5,7-dihydro-4H- |
| | pyrazolo[1,5-a]pyridin-3-yl]-6-methyl-1H-pyrazolo[3,4-b]pyridine; |
| 305 | (*R)-4-[6-Fluoro-2-(5-fluoro-2-pyridyl)-6-(methoxymethyl)-5,7-dihydro-4H- |
| | pyrazolo[1,5-a]pyridin-3-yl]-6-methyl-1H-pyrazolo[3,4-b]pyridine; |
| 306 | (*S)-6-(Difluoromethyl)-4-[6-fluoro-2-(5-fluoro-2-pyridyl)-6-(methoxymethyl)- |
| | 5,7-dihydro-4H-pyrazolo[1,5-a]pyridin-3-yl]-1H-pyrazolo[3,4-b]pyridine; |
| 307 | (*R)-6-(Difluoromethyl)-4-[6-fluoro-2-(5-fluoro-2-pyridyl)-6- |
| | (methoxymethyl)-5,7-dihydro-4H-pyrazolo[1,5-a]pyridin-3-yl]-1H- |
| | pyrazolo[3,4-b]pyridine; |
| 308 | (*S)-5-Fluoro-4-[6-fluoro-2-(5-fluoro-2-pyridyl)-6-(methoxymethyl)-5,7- |
| | dihydro-4H-pyrazolo[1,5-a]pyridin-3-yl]-6-methyl-1H-pyrazolo[3,4-b]pyridine; |
| 309 | (*R)-5-Fluoro-4-[6-fluoro-2-(5-fluoro-2-pyridyl)-6-(methoxymethyl)-5,7- |
| | dihydro-4H-pyrazolo[1,5-a]pyridin-3-yl]-6-methyl-1H-pyrazolo[3,4-b]pyridine; |
| 310 | (*S)- 4-(6-Fluoro-2-(5-fluoropyridin-2-yl)-6-((methoxy-d₃)methyl)-4,5,6,7- |
| | tetrahydropyrazolo[1,5-a]pyridin-3-yl)-6-methyl-1H-pyrazolo[3,4-b]pyridine; |
| 311 | (*R)- 4-(6-Fluoro-2-(5-fluoropyridin-2-yl)-6-((methoxy-d₃)methyl)-4,5,6,7- |
| | tetrahydropyrazolo[1,5-a]pyridin-3-yl)-6-methyl-1H-pyrazolo[3,4-b]pyridine; |
| 312 | (*S)-4-[6-[(Methoxy-d₃)methyl-d₂]-6-fluoro-2-(5-fluoro-2-pyridyl)-5,7-dihydro- |
| | 4H-pyrazolo[1,5-a]pyridin-3-yl]-6-methyl-1H-pyrazolo[3,4-b]pyridine; |
| 313 | (*R)-4-[6-[(Methoxy-d₃)methyl-d₂]-6-fluoro-2-(5-fluoro-2-pyridyl)-5,7- |
| | dihydro-4H-pyrazolo[1,5-a]pyridin-3-yl]-6-methyl-1H-pyrazolo[3,4-b]pyridine; |
| 314 | (*S)-4-(6-((Difluoromethoxy)methyl)-6-fluoro-2-(5-fluoropyridin-2-yl)-4,5,6,7- |
| | tetrahydropyrazolo[1,5-a]pyridin-3-yl)-1H-pyrazolo[3,4-b]pyridine; |
| 315 | (*R)-4-(6-((Difluoromethoxy)methyl)-6-fluoro-2-(5-fluoropyridin-2-yl)-4,5,6,7- |
| | tetrahydropyrazolo[1,5-a]pyridin-3-yl)-1H-pyrazolo[3,4-b]pyridine; |
| 316 | (*S)-4-[6-Fluoro-2-(5-fluoro-2-pyridyl)-6-(trifluoromethoxymethyl)-5,7- |
| | dihydro-4H-pyrazolo[1,5-a]pyridin-3-yl]-1H-pyrazolo[3,4-b]pyridine; |
| 317 | (*R)-4-[6-Fluoro-2-(5-fluoro-2-pyridyl)-6-(trifluoromethoxymethyl)-5,7- |
| | dihydro-4H-pyrazolo[1,5-a]pyridin-3-yl]-1H-pyrazolo[3,4-b]pyridine; |
| 318 | (*S)-4-[2-(5-Fluoro-2-pyridyl)-6-(2-methoxyethyl)-6-methyl-5,7-dihydro-4H- |
| | pyrazolo[1,5-a]pyridin-3-yl]-1H-pyrazolo[3,4-b]pyridine; |
| 319 | (*R)-4-[2-(5-Fluoro-2-pyridyl)-6-(2-methoxyethyl)-6-methyl-5,7-dihydro-4H- |
| | pyrazolo[1,5-a]pyridin-3-yl]-1H-pyrazolo[3,4-b]pyridine; |
| 320 | (*R)-4-(2-(5-Fluoropyridin-2-yl)-6-methyl-6-(oxetan-3-ylmethyl)-4,5,6,7- |
| | tetrahydropyrazolo[1,5-a]pyridin-3-yl)-1H-pyrazolo[3,4-b]pyridine; |
| 321 | (*S)-4-(2-(5-Fluoropyridin-2-yl)-6-methyl-6-(oxetan-3-ylmethyl)-4,5,6,7- |
| | tetrahydropyrazolo[1,5-a]pyridin-3-yl)-1H-pyrazolo[3,4-b]pyridine; |
| 322 | (Racemic) 2-(5-Fluoropyridin-2-yl)-6-(methyl-d₃)-3-(1H-pyrazolo[3,4- |
| | b]pyridin-4-yl)-4,5,6,7-tetrahydropyrazolo[1,5-a]pyridine-6-carbonitrile; |
| 323 | (*S)-2-(5-Fluoropyridin-2-yl)-6-(methyl-d₃)-3-(1H-pyrazolo[3,4-b]pyridin-4- |
| | yl)-4,5,6,7-tetrahydropyrazolo[1,5-a]pyridine-6-carbonitrile; |
| 324 | (*R)-2-(5-Fluoropyridin-2-yl)-6-(methyl-d₃)-3-(1H-pyrazolo[3,4-b]pyridin-4- |
| | yl)-4,5,6,7-tetrahydropyrazolo[1,5-a]pyridine-6-carbonitrile; |
| 325 | (*R)-2,2-Difluoro-2′-(4-fluorophenyl)-3′-(1H-pyrazolo[3,4-b]pyridin-4-yl)-4′,5′- |
| | dihydro-7′H-spiro[cyclopropane-1,6′-pyrazolo[1,5-a]pyridine]; |
| 326 | (*S)-2,2-Difluoro-2′-(4-fluorophenyl)-3′-(1H-pyrazolo[3,4-b]pyridin-4-yl)-4′,5′- |
| | dihydro-7′H-spiro[cyclopropane-1,6′-pyrazolo[1,5-a]pyridine]; |
| 327 | (*S)-2,2-Difluoro-2′-(5-fluoropyridin-2-yl)-3′-(1H-pyrazolo[3,4-b]pyridin-4-yl)- |
| | 4′,5′-dihydro-7′H-spiro[cyclopropane-1,6′-pyrazolo[1,5-a]pyridine]; |
| 328 | (*R)-2,2-Difluoro-2′-(5-fluoropyridin-2-yl)-3′-(1H-pyrazolo[3,4-b]pyridin-4- |
| | yl)-4′,5′-dihydro-7′H-spiro[cyclopropane-1,6′-pyrazolo[1,5-a]pyridine]; |
| 329 | (*S)-2,2-Difluoro-3′-(5-fluoro-1H-pyrazolo[3,4-b]pyridin-4-yl)-2′-(5- |
| | fluoropyridin-2-yl)-4′,5′-dihydro-7′H-spiro[cyclopropane-1,6′-pyrazolo[1,5- |
| | a]pyridine]; |
| 330 | (*R)-2,2-Difluoro-3′-(5-fluoro-1H-pyrazolo[3,4-b]pyridin-4-yl)-2′-(5- |
| | fluoropyridin-2-yl)-4′,5′-dihydro-7′H-spiro[cyclopropane-1,6′-pyrazolo[1,5- |
| | a]pyridine; |
| 331 | (*S)-2,2-Difluoro-3′-(5-fluoro-6-methyl-1H-pyrazolo[3,4-b]pyridin-4-yl)-2′-(5- |
| | fluoropyridin-2-yl)-4′,5′-dihydro-7′H-spiro[cyclopropane-1,6′-pyrazolo[1,5- |
| | alpyridine]; |
| 332 | (*R)-2,2-Difluoro-3′-(5-fluoro-6-methyl-1H-pyrazolo[3,4-b]pyridin-4-yl)-2′-(5- |
| | fluoropyridin-2-yl)-4′,5′-dihydro-7′H-spiro[cyclopropane-1,6′-pyrazolo[1,5- |
| | alpyridine]; |
| 333 | (*S)-2,2-Difluoro-3′-(5-fluoro-3,6-dimethyl-1H-pyrazolo[3,4-b]pyridin-4-yl)-2′- |
| | (5-fluoropyridin-2-yl)-4′,5′-dihydro-7′H-spiro[cyclopropane-1,6′-pyrazolo[1,5- |
| | alpyridine]; |
| 334 | (*R)-2,2-Difluoro-3′-(5-fluoro-3,6-dimethyl-1H-pyrazolo[3,4-b]pyridin-4-y])- |
| | 2′-(5-fluoropyridin-2-yl)-4′,5′-dihydro-7′H-spiro[cyclopropane-1,6′- |
| | pyrazolo[1,5-a]pyridine]; |
| 335 | (*S)-2,2-Difluoro-2′-(4-fluorophenyl)-3′-(6-methyl-1H-pyrazolo[3,4- |
| | d]pyrimidin-4-yl)-4′,5′-dihydro-7′H-spiro[cyclopropane-1,6′-pyrazolo[1,5- |
| | alpyridine]; |
| 336 | (*R)-2,2-Difluoro-2′-(4-fluorophenyl)-3′-(6-methyl-1H-pyrazolo[3,4- |
| | d]pyrimidin-4-yl)-4′,5′-dihydro-7′H-spiro[cyclopropane-1,6′-pyrazolo[1,5- |
| | alpyridine]; |
| 337 | (1*S,4′*S)-4′-Chloro-2,2-difluoro-2′-(5-fluoropyridin-2-yl)-3′-(1H-pyrazolo[3,4- |
| | b]pyridin-4-yl)-4′,5′-dihydro-7′H-spiro[cyclopropane-1,6′-pyrazolo[1,5- |
| | a]pyridine]; |
| 338 | (1*S,4′*R)-4′-Chloro-2,2-difluoro-2′-(5-fluoropyridin-2-yl)-3′-(1H- |
| | pyrazolo[3,4-b]pyridin-4-yl)-4′,5′-dihydro-7′H-spiro[cyclopropane-1,6′- |
| | pyrazolo[1,5-a]pyridine]; |
| 339 | (5a*S,6a*R)-2-(4-Fluorophenyl)-3-(1H-pyrazolo[3,4-b]pyridin-4-yl)-5,5a,6,6a- |
| | tetrahydro-4H-cyclopropa[e]pyrazolo[1,5-a]pyridine; |
| 340 | (5a*R,6a*S)-2-(4-Fluorophenyl)-3-(1H-pyrazolo[3,4-b]pyridin-4-yl)-5,5a,6,6a- |
| | tetrahydro-4H-cyclopropa[e]pyrazolo[1,5-a]pyridine; |
| 341 | N-(4-((5a*R,6a*S)-2-(5-Fluoropyridin-2-yl)-5,5a,6,6a-tetrahydro-4H- |
| | cyclopropa[e]pyrazolo[1,5-a]pyridin-3-yl)pyridin-2-yl)acetamide; |
| 342 | N-(4-((5a*S,6a*R)-2-(5-Fluoropyridin-2-yl)-5,5a,6,6a-tetrahydro-4H- |
| | cyclopropa[e]pyrazolo[1,5-a]pyridin-3-yl)pyridin-2-yl)acetamide; |
| 343 | (5a*R,6a*S)-3-(5-Fluoro-1H-pyrazolo[3,4-b]pyridin-4-yl)-2-(5-fluoropyridin-2- |
| | yl)-5,5a,6,6a-tetrahydro-4H-cyclopropa[e]pyrazolo[1,5-a]pyridine; |
| 344 | (5a*R,6a*S)-3-(5-Fluoro-6-methyl-1H-pyrazolo[3,4-b]pyridin-4-yl)-2-(5- |
| | fluoropyridin-2-yl)-5,5a,6,6a-tetrahydro-4H-cyclopropa[e]pyrazolo[1,5- |
| | alpyridine; |
| 345 | (5a*R,6a*S)-3-(5-Fluoro-3-methyl-1H-pyrazolo[3,4-b]pyridin-4-yl)-2-(5- |
| | fluoropyridin-2-yl)-5,5a,6,6a-tetrahydro-4H-cyclopropa[e]pyrazolo[1,5- |
| | alpyridine; |
| 346 | (5a*R,6a*S)-3-(5-Fluoro-3,6-dimethyl-1H-pyrazolo[3,4-b]pyridin-4-yl)-2-(5- |
| | fluoropyridin-2-yl)-5,5a,6,6a-tetrahydro-4H-cyclopropa[e]pyrazolo[1,5- |
| | a]pyridine; |
| 347 | (5a*R,6a*S)-2-(5-Fluoropyridin-2-yl)-3-(6-methyl-1H-pyrazolo[3,4- |
| | d]pyrimidin-4-yl)-5,5a,6,6a-tetrahydro-4H-cyclopropa[e]pyrazolo[1,5- |
| | alpyridine; |
| 348 | (Racemic) N-(4-(6,6-Difluoro-2-(4-fluorophenyl)-5,5a,6,6a-tetrahydro-4H- |
| | cyclopropa[e]pyrazolo[1,5-a]pyridin-3-yl)pyridin-2-yl)acetamide; |
| 349 | N-(4-((5a*R,6a*S)-6,6-Difluoro-2-(4-fluorophenyl)-5,5a,6,6a-tetrahydro-4H- |
| | cyclopropa[e]pyrazolo[1,5-a]pyridin-3-yl)pyridin-2-yl)acetamide; |
| 350 | N-(4-((5a*S,6a*R)-6,6-Difluoro-2-(4-fluorophenyl)-5,5a,6,6a-tetrahydro-4H- |
| | cyclopropa[e]pyrazolo[1,5-a]pyridin-3-yl)pyridin-2-yl)acetamide; |
| 351 | N-(4-((5a*S,6a*R)-6,6-Difluoro-2-(5-fluoropyridin-2-yl)-5,5a,6,6a-tetrahydro- |
| | 4H-cyclopropa[e]pyrazolo[1,5-a]pyridin-3-yl)pyridin-2-yl)acetamide; |
| 352 | N-(4-((5a*R,6a*S)-6,6-Difluoro-2-(5-fluoropyridin-2-yl)-5,5a,6,6a-tetrahydro- |
| | 4H-cyclopropa[e]pyrazolo[1,5-a]pyridin-3-yl)pyridin-2-yl)acetamide; |
| 353 | (5a*S,6a*R)-6,6-Difluoro-2-(4-fluorophenyl)-3-(pyrazolo[1,5-a]pyridin-4-yl)- |
| | 5,5a,6,6a-tetrahydro-4H-cyclopropa[e]pyrazolo[1,5-a]pyridine; |
| 354 | (5a*R,6a*S)-6,6-Difluoro-2-(4-fluorophenyl)-3-(pyrazolo[1,5-a]pyridin-4-yl)- |
| | 5,5a,6,6a-tetrahydro-4H-cyclopropa[e]pyrazolo[1,5-a]pyridine; |
| 355 | (5a*S,6a*R)-6,6-Difluoro-2-(4-fluorophenyl)-3-(1H-pyrazolo[3,4-b]pyridin-4- |
| | yl)-5,5a,6,6a-tetrahydro-4H-cyclopropa[e]pyrazolo[1,5-a]pyridine; |
| 356 | (5a*R,6a*S)-6,6-Difluoro-2-(4-fluorophenyl)-3-(1H-pyrazolo[3,4-b]pyridin-4- |
| | yl)-5,5a,6,6a-tetrahydro-4H-cyclopropa[e]pyrazolo[1,5-a]pyridine; |
| 357 | (5a*S,6a*R)-6,6-Difluoro-3-(5-fluoro-6-methyl-1H-pyrazolo[3,4-b]pyridin-4- |
| | yl)-2-(5-fluoropyridin-2-yl)-5,5a,6,6a-tetrahydro-4H- |
| | cyclopropa[e]pyrazolo[1,5-a]pyridine; |
| 358 | (5a*S,6a*R)-6,6-Difluoro-3-(5-fluoro-1H-pyrazolo[3,4-b]pyridin-4-yl)-2-(5- |
| | fluoropyridin-2-yl)-5,5a,6,6a-tetrahydro-4H-cyclopropa[e]pyrazolo[1,5- |
| | alpyridine; |
| 359 | (5a*S,6a*R)-6,6-Difluoro-3-(5-fluoro-3-methyl-1H-pyrazolo[3,4-b]pyridin-4- |
| | yl)-2-(5-fluoropyridin-2-yl)-5,5a,6,6a-tetrahydro-4H- |
| | cyclopropa[e]pyrazolo[1,5-a]pyridine; |
| 360 | (5a*S,6a*R)-6,6-Difluoro-3-(5-fluoro-3,6-dimethyl-1H-pyrazolo[3,4-b]pyridin- |
| | 4-yl)-2-(5-fluoropyridin-2-yl)-5,5a,6,6a-tetrahydro-4H- |
| | cyclopropa[e]pyrazolo[1,5-a]pyridine; |
| 361 | (5a*S,6a*R)-6,6-Difluoro-2-(5-fluoropyridin-2-yl)-3-(6-methyl-1H- |
| | pyrazolo[3,4-d]pyrimidin-4-yl)-5,5a,6,6a-tetrahydro-4H- |
| | cyclopropa[e]pyrazolo[1,5-a]pyridine; |
| 362 | (4a*R,5a*R)-5,5-Difluoro-2-(4-fluorophenyl)-3-(1H-pyrazolo[3,4-b]pyridin-4- |
| | yl)-4a,5,5a,6-tetrahydro-4H-cyclopropa[d]pyrazolo[1,5-a]pyridine; |
| 363 | (4a*S,5a*S)-5,5-Difluoro-2-(4-fluorophenyl)-3-(1H-pyrazolo[3,4-b]pyridin-4- |
| | yl)-4a,5,5a,6-tetrahydro-4H-cyclopropa[d]pyrazolo[1,5-a]pyridine; |
| 364 | (4*R,7*S)-2-(4-Fluorophenyl)-3-(6-methyl-1H-pyrazolo[3,4-b]pyridin-4-yl)- |
| | 4,5,6,7-tetrahydro-4,7-methanopyrazolo[1,5-a]pyridine; |
| 365 | (4*S,7*R)-2-(4-Fluorophenyl)-3-(6-methyl-1H-pyrazolo[3,4-b]pyridin-4-yl)- |
| | 4,5,6,7-tetrahydro-4,7-methanopyrazolo[1,5-a]pyridine; |
| 366 | 2-(5-Fluoropyridin-2-yl)-6,6-bis(methyl-d₃)-3-(1H-pyrazolo[3,4-b]pyridin-4- |
| | yl)-4,5,6,7-tetrahydropyrazolo[1,5-a]pyridin-4-ol; |
| 367 | 6-(5-Fluoro-2-pyridyl)-2,2-dimethyl-7-pyrazolo[1,5-a]pyridin-5-yl-3H- |
| | pyrazolo[5,1-b]oxazole; |
| 368 | 6-(5-Fluoro-2-pyridyl)-2,2-dimethyl-7-(6-methyl-1H-pyrazolo[3,4-b]pyridin-4- |
| | yl)-3H-pyrazolo[5,1-b]oxazole; |
| 369 | 2-(4-Fluorophenyl)-3-(4-pyridyl)-6,7-dihydro-4H-pyrazolo[5,1-c][1,4]oxazine; |
| 370 | 3-(1-Ethyl-1H-pyrazolo[3,4-b]pyridin-5-yl)-2-(4-fluorophenyl)-7-methyl-6,7- |
| | dihydro-4H-pyrazolo[5,1-c][1,4]oxazine; |
| 371 | (S)-3-(1-Ethyl-1H-pyrazolo[3,4-b]pyridin-5-yl)-2-(5-fluoropyridin-2-yl)-6- |
| | methyl-6,7-dihydro-4H-pyrazolo[5,1-c][1,4]oxazine; |
| 372 | (*S)-2-(4-Fluorophenyl)-3-(3-methyl-1H-pyrazolo[3,4-b]pyridin-4-yl)-6- |
| | (trifluoromethyl)-6,7-dihydro-4H-pyrazolo[5,1-c][1,4]oxazine; |
| 373 | (*R)-2-(4-Fluorophenyl)-3-(3-methyl-1H-pyrazolo[3,4-b]pyridin-4-yl)-6- |
| | (trifluoromethyl)-6,7-dihydro-4H-pyrazolo[5,1-c][1,4]oxazine; |
| 374 | (*S)-3-[6-(Difluoromethyl)-3-methyl-1H-pyrazolo[3,4-b]pyridin-4-yl]-2-(4- |
| | fluorophenyl)-6-(trifluoromethyl)-6,7-dihydro-4H-pyrazolo[5,1-c][1,4]oxazine; |
| 375 | (*R)-3-[6-(Difluoromethyl)-3-methyl-1H-pyrazolo[3,4-b]pyridin-4-yl]-2-(4- |
| | fluorophenyl)-6-(trifluoromethyl)-6,7-dihydro-4H-pyrazolo[5,1-c][1,4]oxazine; |
| 376 | (*R)-2-(4-Fluorophenyl)-3-(6-methyl-1H-pyrazolo[3,4-d]pyrimidin-4-yl)-6- |
| | (trifluoromethyl)-6,7-dihydro-4H-pyrazolo[5,1-c][1,4]oxazine; |
| 377 | (*S)-2-(4-Fluorophenyl)-3-(6-methyl-1H-pyrazolo[3,4-d]pyrimidin-4-yl)-6- |
| | (trifluoromethyl)-6,7-dihydro-4H-pyrazolo[5,1-c][1,4]oxazine; |
| 378 | 4-(2-(5-Fluoropyridin-2-yl)-6,6-dimethyl-6,7-dihydro-4H-pyrazolo[5,1- |
| | c][1,4]oxazin-3-yl)-5-methylpyridin-2-amine; |
| 379 | N-(4-(2-(5-Fluoropyridin-2-yl)-6,6-dimethyl-6,7-dihydro-4H-pyrazolo[5,1- |
| | c][1,4]oxazin-3-yl)-5-methylpyridin-2-yl)propionamide; |
| 380 | 5-[2-(5-Fluoro-2-pyridyl)-6,6-dimethyl-4,7-dihydropyrazolo[5,1-c][1,4]oxazin- |
| | 3-yl]pyrazolo[1,5-a]pyridin-7-amine; |
| 381 | 5-[2-(5-Fluoro-2-pyridyl)-6,6-dimethyl-4,7-dihydropyrazolo[5,1-c][1,4]oxazin- |
| | 3-yl]pyrazolo[1,5-a]pyridin-3-amine; |
| 382 | 2-(5-Fluoro-2-pyridyl)-6,6-dimethyl-3-(6-methylpyrazolo[1,5-a]pyridin-5-yl)- |
| | 4,7-dihydropyrazolo[5,1-c][1,4]oxazine; |
| 383 | 2-(5-Fluoro-2-pyridyl)-6,6-dimethyl-3-(3-methylpyrazolo[1,5-a]pyridin-5-yl)- |
| | 4,7-dihydropyrazolo[5,1-c][1,4]oxazine; |
| 384 | 3-(3-Chloropyrazolo[1,5-a]pyridin-5-yl)-2-(5-fluoropyridin-2-yl)-6,6-dimethyl- |
| | 6,7-dihydro-4H-pyrazolo[5,1-c][1,4]oxazine; |
| 385 | 3-([1,2,4]Triazolo[1,5-a]pyridin-7-yl)-2-(5-fluoropyridin-2-yl)-6,6-dimethyl- |
| | 6,7-dihydro-4H-pyrazolo[5,1-c][1,4]oxazine; |
| 386 | 2-(5-Fluoropyridin-2-yl)-6,6-dimethyl-3-(2-methyl-[1,2,4]triazolo[1,5- |
| | a]pyridin-7-yl)-6,7-dihydro-4H-pyrazolo[5,1-c][1,4]oxazine; |
| 387 | 2-(5-Fluoropyridin-2-yl)-6,6-dimethyl-3-(pyrazolo[1,5-a]pyrimidin-5-yl)-6,7- |
| | dihydro-4H-pyrazolo[5,1-c][1,4]oxazine; |
| 388 | 3-(3-Chloro-1H-pyrazolo[3,4-b]pyridin-4-yl)-2-(5-fluoropyridin-2-yl)-6,6- |
| | dimethyl-6,7-dihydro-4H-pyrazolo[5,1-c][1,4]oxazine; |
| 389 | 3-(5-Fluoro-1H-pyrazolo[3,4-b]pyridin-4-yl)-2-(5-fluoropyridin-2-yl)-6,6- |
| | dimethyl-6,7-dihydro-4H-pyrazolo[5,1-c][1,4]oxazine; |
| 390 | 3-[6-(Difluoromethyl)-1H-pyrazolo[3,4-b]pyridin-4-yl]-2-(5-fluoro-2-pyridyl)- |
| | 6,6-dimethyl-4,7-dihydropyrazolo[5,1-c][1,4]oxazine; |
| 391 | 2-(5-Fluoro-2-pyridyl)-6,6-dimethyl-3-[6-(3,3,3-trifluoropropyl)-1H- |
| | pyrazolo[3,4-b]pyridin-4-yl]-4,7-dihydropyrazolo[5,1-c][1,4]oxazine; |
| 392 | 3-(3-Chloro-5-fluoro-1H-pyrazolo[3,4-b]pyridin-4-yl)-2-(5-fluoropyridin-2-y])- |
| | 6,6-dimethyl-6,7-dihydro-4H-pyrazolo[5,1-c][1,4]oxazine; |
| 393 | 3-(3,6-Dimethyl-1H-pyrazolo[3,4-b]pyridin-4-yl)-2-(5-fluoro-6-methylpyridin- |
| | 2-yl)-6,6-dimethyl-6,7-dihydro-4H-pyrazolo[5,1-c][1,4]oxazine; |
| 394 | 2-(4-Fluorophenyl)-6,6-dimethyl-3-(6-methyl-1H-pyrazolo[3,4-d]pyrimidin-4- |
| | yl)-4,7-dihydropyrazolo[5,1-c][1,4]oxazine; |
| 395 | N-(4-(2-(5-Fluoropyridin-2-yl)-6,6-bis(methyl-d₃)-6,7-dihydro-4H- |
| | pyrazolo[5,1-c][1,4]oxazin-3-yl)pyridin-2-yl)propionamide; |
| 396 | 2-(5-Fluoropyridin-2-yl)-6,6-bis(methyl-d₃)-3-(pyrazolo[1,5-a]pyridin-5-y])- |
| | 6,7-dihydro-4H-pyrazolo[5,1-c][1,4]oxazine; |
| 397 | 3-(5-Fluoro-1H-pyrrolo[2,3-b]pyridin-4-yl)-2-(5-fluoropyridin-2-yl)-6,6- |
| | bis(methyl-d₃)-6,7-dihydro-4H-pyrazolo[5,1-c][1,4]oxazine; |
| 398 | 3-(3-Fluoro-1H-pyrazolo[3,4-b]pyridin-4-yl)-2-(5-fluoropyridin-2-yl)-6,6- |
| | bis(methyl-d₃)-6,7-dihydro-4H-pyrazolo[5,1-c][1,4]oxazine; |
| 399 | 3-(5-Fluoro-1H-pyrazolo[3,4-b]pyridin-4-yl)-2-(5-fluoropyridin-2-yl)-6,6- |
| | bis(methyl-d₃)-6,7-dihydro-4H-pyrazolo[5,1-c][1,4]oxazine; |
| 400 | 3-(3-Chloro-1H-pyrazolo[3,4-b]pyridin-4-yl)-2-(5-fluoropyridin-2-yl)-6,6- |
| | bis(methyl-d₃)-6,7-dihydro-4H-pyrazolo[5,1-c][1,4]oxazine; |
| 401 | 2-(5-Fluoropyridin-2-yl)-6,6-bis(methyl-d₃)-3-(6-methyl-1H-pyrazolo[3,4- |
| | b]pyridin-4-yl)-6,7-dihydro-4H-pyrazolo[5,1-c][1,4]oxazine; |
| 402 | 3-(6-(Difluoromethyl)-1H-pyrazolo[3,4-b]pyridin-4-yl)-2-(5-fluoropyridin-2- |
| | yl)-6,6-bis(methyl-d₃)-6,7-dihydro-4H-pyrazolo[5,1-c][1,4]oxazine; |
| 403 | 3-(3-Chloro-5-fluoro-1H-pyrazolo[3,4-b]pyridin-4-yl)-2-(5-fluoropyridin-2-y])- |
| | 6,6-bis(methyl-d₃)-6,7-dihydro-4H-pyrazolo[5,1-c][1,4]oxazine; |
| 404 | 3-(5-Fluoro-3-methyl-1H-pyrazolo[3,4-b]pyridin-4-yl)-2-(5-fluoropyridin-2-yl)- |
| | 6,6-bis(methyl-d₃)-6,7-dihydro-4H-pyrazolo[5,1-c][1,4]oxazine; |
| 405 | 3-(3-Chloro-6-methyl-1H-pyrazolo[3,4-b]pyridin-4-yl)-2-(5-fluoropyridin-2- |
| | yl)-6,6-bis(methyl-d₃)-6,7-dihydro-4H-pyrazolo[5,1-c][1,4]oxazine; |
| 406 | 3-(5-Fluoro-6-methyl-1H-pyrazolo[3,4-b]pyridin-4-yl)-2-(5-fluoropyridin-2-yl)- |
| | 6,6-bis(methyl-d₃)-6,7-dihydro-4H-pyrazolo[5,1-c][1,4]oxazine; |
| 407 | 3-(5-Chloro-6-methyl-1H-pyrazolo[3,4-b]pyridin-4-yl)-2-(5-fluoropyridin-2- |
| | yl)-6,6-bis(methyl-d₃)-6,7-dihydro-4H-pyrazolo[5,1-c][1,4]oxazine; |
| 408 | 3-(3-Chloro-5-fluoro-6-methyl-1H-pyrazolo[3,4-b]pyridin-4-yl)-2-(5- |
| | fluoropyridin-2-yl)-6,6-bis(methyl-d₃)-6,7-dihydro-4H-pyrazolo[5,1- |
| | c][1,4]oxazine; |
| 409 | 3-(5-Fluoro-3,6-dimethyl-1H-pyrazolo[3,4-b]pyridin-4-yl)-2-(5-fluoropyridin- |
| | 2-yl)-6,6-bis(methyl-d₃)-6,7-dihydro-4H-pyrazolo[5,1-c][1,4]oxazine; |
| 410 | 2-(4-Fluorophenyl)-6,6-bis(methyl-d₃)-3-(6-methyl-1H-pyrazolo[3,4- |
| | d]pyrimidin-4-yl)-6,7-dihydro-4H-pyrazolo[5,1-c][1,4]oxazine; |
| 411 | 2-(5-Fluoropyridin-2-yl)-6,6-bis(methyl-d₃)-3-(1H-pyrazolo[3,4-b]pyridin-4- |
| | yl)-6,7-dihydro-4H-pyrazolo[5,1-c][1,4]oxazine-4,4,7,7-d₄; |
| 412 | (*R)-2-(4-Fluorophenyl)-6-methyl-3-(pyrazolo[1,5-a]pyridin-5-yl)-6- |
| | (trifluoromethyl)-6,7-dihydro-4H-pyrazolo[5,1-c][1,4]oxazine; |
| 413 | (*S)-2-(4-Fluorophenyl)-6-methyl-3-(pyrazolo[1,5-a]pyridin-5-yl)-6- |
| | (trifluoromethyl)-6,7-dihydro-4H-pyrazolo[5,1-c][1,4]oxazine; |
| 414 | (*R)-2-(5-Fluoropyridin-2-yl)-6-methyl-3-(pyrazolo[1,5-a]pyridin-5-yl)-6- |
| | (trifluoromethyl)-6,7-dihydro-4H-pyrazolo[5,1-c][1,4]oxazine; |
| 415 | (*S)-2-(5-Fluoropyridin-2-yl)-6-methyl-3-(pyrazolo[1,5-a]pyridin-5-yl)-6- |
| | (trifluoromethyl)-6,7-dihydro-4H-pyrazolo[5,1-c][1,4]oxazine; |
| 416 | (*R)-2-(4-Fluorophenyl)-6-methyl-3-(1H-pyrazolo[3,4-b]pyridin-4-yl)-6- |
| | (trifluoromethyl)-6,7-dihydro-4H-pyrazolo[5,1-c][1,4]oxazine; |
| 417 | (*S)-2-(4-Fluorophenyl)-6-methyl-3-(1H-pyrazolo[3,4-b]pyridin-4-yl)-6- |
| | (trifluoromethyl)-6,7-dihydro-4H-pyrazolo[5,1-c][1,4]oxazine; |
| 418 | (*R)-2-(5-Fluoropyridin-2-yl)-6-methyl-3-(3-methyl-1H-pyrazolo[3,4- |
| | b]pyridin-4-yl)-6-(trifluoromethyl)-6,7-dihydro-4H-pyrazolo[5,1- |
| | c][1,4]oxazine; |
| 419 | (*S)-2-(5-Fluoropyridin-2-yl)-6-methyl-3-(3-methyl-1H-pyrazolo[3,4- |
| | b]pyridin-4-yl)-6-(trifluoromethyl)-6,7-dihydro-4H-pyrazolo[5,1- |
| | c][1,4]oxazine; |
| 420 | (*R)-3-(5-Fluoro-6-methyl-1H-pyrazolo[3,4-b]pyridin-4-yl)-2-(5-fluoropyridin- |
| | 2-yl)-6-methyl-6-(trifluoromethyl)-6,7-dihydro-4H-pyrazolo[5,1- |
| | c][1,4]oxazine; |
| 421 | (*S)-3-(5-Fluoro-6-methyl-1H-pyrazolo[3,4-b]pyridin-4-yl)-2-(5-fluoropyridin- |
| | 2-yl)-6-methyl-6-(trifluoromethyl)-6,7-dihydro-4H-pyrazolo[5,1- |
| | c][1,4]oxazine; |
| 422 | 2-(4-Fluorophenyl)-6-methyl-3-(6-methyl-1H-pyrazolo[3,4-d]pyrimidin-4-yl)- |
| | 6-(trifluoromethyl)-6,7-dihydro-4H-pyrazolo[5,1-c][1,4]oxazine; |
| 423 | (*R)-2-(5-Fluoropyridin-2-yl)-6-(methyl-d₃)-3-(1H-pyrazolo[3,4-b]pyridin-4- |
| | yl)-6-(trifluoromethyl)-6,7-dihydro-4H-pyrazolo[5,1-c][1,4]oxazine; |
| 424 | (*S)-2-(5-Fluoropyridin-2-yl)-6-(methyl-d₃)-3-(1H-pyrazolo[3,4-b]pyridin-4- |
| | yl)-6-(trifluoromethyl)-6,7-dihydro-4H-pyrazolo[5,1-c][1,4]oxazine; |
| 425 | (*R)-2-(5-Fluoropyridin-2-yl)-6-(methyl-d₃)-3-(3-methyl-1H-pyrazolo[3,4- |
| | b]pyridin-4-yl)-6-(trifluoromethyl)-6,7-dihydro-4H-pyrazolo[5,1- |
| | c][1,4]oxazine; |
| 426 | (*S)-2-(5-Fluoropyridin-2-yl)-6-(methyl-d₃)-3-(3-methyl-1H-pyrazolo[3,4- |
| | b]pyridin-4-yl)-6-(trifluoromethyl)-6,7-dihydro-4H-pyrazolo[5,1- |
| | c][1,4]oxazine; |
| 427 | (*S)-3-(3-Chloro-1H-pyrazolo[3,4-b]pyridin-4-yl)-2-(5-fluoropyridin-2-yl)-6- |
| | (methyl-d₃)-6-(trifluoromethyl)-6,7-dihydro-4H-pyrazolo[5,1-c][1,4]oxazine; |
| 428 | (*R)-3-(3,6-Dimethyl-1H-pyrazolo[3,4-b]pyridin-4-yl)-2-(5-fluoropyridin-2- |
| | yl)-6-(methyl-d₃)-6-(trifluoromethyl)-6,7-dihydro-4H-pyrazolo[5,1- |
| | c][1,4]oxazine; |
| 429 | (*S)-3-(3,6-Dimethyl-1H-pyrazolo[3,4-b]pyridin-4-yl)-2-(5-fluoropyridin-2-y])- |
| | 6-(methyl-d₃)-6-(trifluoromethyl)-6,7-dihydro-4H-pyrazolo[5,1-c][1,4]oxazine; |
| 430 | (*R)-3-(5-Fluoro-3,6-dimethyl-1H-pyrazolo[3,4-b]pyridin-4-yl)-2-(5- |
| | fluoropyridin-2-yl)-6-(methyl-d₃)-6-(trifluoromethyl)-6,7-dihydro-4H- |
| | pyrazolo[5,1-c][1,4]oxazine; |
| 431 | (*S)-3-(5-Fluoro-3,6-dimethyl-1H-pyrazolo[3,4-b]pyridin-4-yl)-2-(5- |
| | fluoropyridin-2-yl)-6-(methyl-d₃)-6-(trifluoromethyl)-6,7-dihydro-4H- |
| | pyrazolo[5,1-c][1,4]oxazine; |
| 432 | 2-(5-Fluoro-2-pyridyl)-3-(1H-pyrazolo[3,4-b]pyridin-4-yl)spiro[4,7- |
| | dihydropyrazolo[5,1-c][1,4]oxazine-6,1′-cyclopropane]; |
| 433 | (*R)-1′,1′-Difluoro-2-(5-fluoro-2-pyridyl)-3-(1H-pyrazolo[3,4-b]pyridin-4- |
| | yl)spiro[4,7-dihydropyrazolo[5,1-c][1,4]oxazine-6,2′-cyclopropane]; |
| 434 | (*S)-1′,1′-Difluoro-2-(5-fluoro-2-pyridyl)-3-(1H-pyrazolo[3,4-b]pyridin-4- |
| | yl)spiro[4,7-dihydropyrazolo[5,1-c][1,4]oxazine-6,2′-cyclopropane]; |
| 435 | (*R)-1′,1′-Difluoro-3-(5-fluoro-1H-pyrazolo[3,4-b]pyridin-4-yl)-2-(5-fluoro-2- |
| | pyridyl)spiro[4,7-dihydropyrazolo[5,1-c][1,4]oxazine-6,2′-cyclopropane]; |
| 436 | (*S)-1′,1′-Difluoro-3-(5-fluoro-1H-pyrazolo[3,4-b]pyridin-4-yl)-2-(5-fluoro-2- |
| | pyridyl)spiro[4,7-dihydropyrazolo[5,1-c][1,4]oxazine-6,2′-cyclopropane]; |
| 437 | (*S)-3-(3-Chloro-1H-pyrazolo[3,4-b]pyridin-4-yl)-2-(5-fluoropyridin-2-yl)- |
| | 6,6,7-trimethyl-6,7-dihydro-4H-pyrazolo[5,1-c][1,4]oxazine; |
| 438 | 2-(4-Fluorophenyl)-3-(4-pyridyl)-6,7-dihydro-5H-pyrazolo[5,1-b][1,3]oxazine; |
| 439 | N-(4-(2-(5-Fluoropyridin-2-yl)-6,7-dihydro-5H-pyrazolo[5,1-b][1,3]oxazin-3- |
| | yl)pyridin-2-yl)propionamide; |
| 440 | 2-(5-Fluoropyridin-2-yl)-3-(pyrazolo[1,5-a]pyridin-5-yl)-6,7-dihydro-5H- |
| | pyrazolo[5,1-b][1,3]oxazine; |
| 441 | 3-(1-Ethyl-1H-pyrazolo[3,4-b]pyridin-5-yl)-2-(5-fluoropyridin-2-yl)-6,7- |
| | dihydro-5H-pyrazolo[5,1-b][1,3]oxazine; |
| 442 | N-(4-(2-(5-Fluoropyridin-2-yl)-6-methyl-6,7-dihydro-5H-pyrazolo[5,1- |
| | b][1,3]oxazin-3-yl)pyridin-2-yl)propionamide; |
| 443 | 2-(5-Fluoropyridin-2-yl)-6-methyl-3-(pyrazolo[1,5-a]pyridin-5-yl)-6,7-dihydro- |
| | 5H-pyrazolo[5,1-b][1,3]oxazine; |
| 444 | 3-(1-Ethyl-1H-pyrazolo[3,4-b]pyridin-5-yl)-2-(5-fluoropyridin-2-yl)-6-methyl- |
| | 6,7-dihydro-5H-pyrazolo[5,1-b][1,3]oxazine; |
| 445 | N-(4-(2-(5-Fluoropyridin-2-yl)-7-methyl-6,7-dihydro-5H-pyrazolo[5,1- |
| | b][1,3]oxazin-3-yl)pyridin-2-yl)propionamide; |
| 446 | 2-(5-Fluoropyridin-2-yl)-7-methyl-3-(pyrazolo[1,5-a]pyridin-5-yl)-6,7-dihydro- |
| | 5H-pyrazolo[5,1-b][1,3]oxazine; |
| 447 | N-(4-(2-(5-Fluoropyridin-2-yl)-6,6-dimethyl-6,7-dihydro-5H-pyrazolo[5,1- |
| | b][1,3]oxazin-3-yl)pyridin-2-yl)propionamide; |
| 448 | 2-(4-Fluorophenyl)-6,6-dimethyl-3-(1H-pyrrolo[3,2-b]pyridin-7-yl)-5,7- |
| | dihydropyrazolo[5,1-b][1,3]oxazine; |
| 449 | 2-(5-Fluoropyridin-2-yl)-6,6-dimethyl-3-(pyrazolo[1,5-a]pyridin-5-yl)-6,7- |
| | dihydro-5H-pyrazolo[5,1-b][1,3]oxazine; |
| 450 | 2-(5-Fluoro-2-pyridyl)-6,6-dimethyl-3-(1H-pyrrolo[3,2-b]pyridin-7-yl)-5,7- |
| | dihydropyrazolo[5,1-b][1,3]oxazine; |
| 451 | 2-(5-Fluoro-2-pyridyl)-6,6-dimethyl-3-(6-methylpyrazolo[1,5-a]pyridin-5-y])- |
| | 5,7-dihydropyrazolo[5,1-b][1,3]oxazine; |
| 452 | 3-(1-Ethyl-1H-pyrazolo[3,4-b]pyridin-5-yl)-2-(5-fluoropyridin-2-yl)-6,6- |
| | dimethyl-6,7-dihydro-5H-pyrazolo[5,1-b][1,3]oxazine; |
| 453 | 2-(4-Fluorophenyl)-6,6-dimethyl-3-(1H-pyrazolo[4,3-b]pyridin-7-yl)-5,7- |
| | dihydropyrazolo[5,1-b][1,3]oxazine; |
| 454 | 2-(5-Fluoro-2-pyridyl)-6,6-dimethyl-3-(1H-pyrazolo[4,3-b]pyridin-7-yl)-5,7- |
| | dihydropyrazolo[5,1-b][1,3]oxazine; |
| 455 | N-(4-(2′-(4-Fluorophenyl)-5′H,7′H-spiro[cyclopropane-1,6′-pyrazolo[5,1- |
| | b][1,3]oxazin]-3′-yl)pyridin-2-yl)propionamide; |
| 456 | N-(4-(2′-(5-Fluoropyridin-2-yl)-5′H,7′H-spiro[cyclopropane-1,6′-pyrazolo[5,1- |
| | b][1,3]oxazin]-3′-yl)pyridin-2-yl)cyclopropanecarboxamide; |
| 457 | 2′-(4-Fluorophenyl)-3′-(1H-pyrazolo[3,4-b]pyridin-4-yl)-5′H,7′H- |
| | spiro[cyclopropane-1,6′-pyrazolo[5,1-b][1,3]oxazine]; |
| 458 | 3′-(3-Chloro-1H-pyrazolo[3,4-b]pyridin-4-yl)-2′-(5-fluoropyridin-2-yl)-5′H,7′H- |
| | spiro[cyclopropane-1,6′-pyrazolo[5,1-b][1,3]oxazine]; |
| 459 | 3′-(5-Fluoro-1H-pyrazolo[3,4-b]pyridin-4-yl)-2′-(5-fluoropyridin-2-yl)-5′H,7′H- |
| | spiro[cyclopropane-1,6′-pyrazolo[5,1-b][1,3]oxazine]; |
| 460 | 2′-(5-Fluoropyridin-2-yl)-3′-(1H-pyrazolo[3,4-b]pyridin-4-yl)-5′H,7′H- |
| | spiro[cyclopropane-1,6′-pyrazolo[5,1-b][1,3]oxazine]; |
| 461 | 2′-(4-Fluorophenyl)-3′-(6-methyl-1H-pyrazolo[3,4-b]pyridin-4-yl)-5′H,7′H- |
| | spiro[cyclopropane-1,6′-pyrazolo[5,1-b][1,3]oxazine]; |
| 462 | 2′-(5-Fluoropyridin-2-yl)-3′-(6-methyl-1H-pyrazolo[3,4-b]pyridin-4-yl)- |
| | 5′H,7′H-spiro[cyclopropane-1,6′-pyrazolo[5,1-b][1,3]oxazine]; |
| 463 | 3′-(5-Fluoro-6-methyl-1H-pyrazolo[3,4-b]pyridin-4-yl)-2′-(5-fluoropyridin-2- |
| | yl)-5′H,7′H-spiro[cyclopropane-1,6′-pyrazolo[5,1-b][1,3]oxazine]; |
| 464 | 3′-(3-Chloro-6-methyl-1H-pyrazolo[3,4-b]pyridin-4-yl)-2′-(5-fluoropyridin-2- |
| | yl)-5′H,7′H-spiro[cyclopropane-1,6′-pyrazolo[5,1-b][1,3]oxazine]; |
| 465 | 2′-(4-Fluorophenyl)-3′-(6-methyl-1H-pyrazolo[3,4-d]pyrimidin-4-yl)-5′H,7′H- |
| | spiro[cyclopropane-1,6′-pyrazolo[5,1-b][1,3]oxazine]; |
| 466 | 2′-(4-Fluorophenyl)-3′-(pyrazolo[1,5-a]pyridin-5-yl)-5′H,7′H- |
| | spiro[cyclopropane-1,6′-pyrazolo[5,1-b][1,3]oxazine]; |
| 467 | 6,6-Difluoro-2-(4-fluorophenyl)-3-(1H-pyrazolo[3,4-b]pyridin-4-yl)-5,7- |
| | dihydropyrazolo[5,1-b][1,3]oxazine; |
| 468 | 6,6-Difluoro-2-(4-fluorophenyl)-3-(1H-pyrazolo[4,3-b]pyridin-7-yl)-5,7- |
| | dihydropyrazolo[5,1-b][1,3]oxazine; |
| 469 | 6,6-Difluoro-2-(4-fluorophenyl)-3-(6-methyl-1H-pyrazolo[3,4-d]pyrimidin-4- |
| | yl)-5,7-dihydropyrazolo[5,1-b][1,3]oxazine; |
| 470 | 3-(3-Chloro-1H-pyrazolo[3,4-b]pyridin-4-yl)-6,6-difluoro-2-(4-fluorophenyl)- |
| | 6,7-dihydro-5H-pyrazolo[5,1-b][1,3]oxazine; |
| 471 | 2,2-Difluoro-3′-(5-fluoro-1H-pyrazolo[3,4-b]pyridin-4-yl)-2′-(4-fluorophenyl)- |
| | 5′H,7′H-spiro[cyclopropane-1,6′-pyrazolo[5,1-b][1,3]oxazine]; |
| 472 | (*S)-2,2-Difluoro-2′-(4-fluorophenyl)-3′-(3-methyl-1H-pyrazolo[3,4-b]pyridin- |
| | 4-yl)-5′H,7′H-spiro[cyclopropane-1,6′-pyrazolo[5,1-b][1,3]oxazine]; |
| 473 | (*R)-2,2-Difluoro-2′-(4-fluorophenyl)-3′-(3-methyl-1H-pyrazolo[3,4-b]pyridin- |
| | 4-yl)-5′H,7′H-spiro[cyclopropane-1,6′-pyrazolo[5,1-b][1,3]oxazine]; |
| 474 | (*S)-2,2-Difluoro-2′-(4-fluorophenyl)-3′-(6-methyl-1H-pyrazolo[3,4-b]pyridin- |
| | 4-yl)-5′H,7′H-spiro[cyclopropane-1,6′-pyrazolo[5,1-b][1,3]oxazine]; |
| 475 | (*R)-2,2-Difluoro-2′-(4-fluorophenyl)-3′-(6-methyl-1H-pyrazolo[3,4-b]pyridin- |
| | 4-yl)-5′H,7′H-spiro[cyclopropane-1,6′-pyrazolo[5,1-b][1,3]oxazine]; |
| 476 | (*S)-2,2-Difluoro-2′-(4-fluorophenyl)-3′-(1H-pyrazolo[3,4-b]pyridin-4-y])- |
| | 5′H,7′H-spiro[cyclopropane-1,6′-pyrazolo[5,1-b][1,3]oxazine]; |
| 477 | (*R)-2,2-Difluoro-2′-(4-fluorophenyl)-3′-(1H-pyrazolo[3,4-b]pyridin-4-yl)- |
| | 5′H,7′H-spiro[cyclopropane-1,6′-pyrazolo[5,1-b][1,3]oxazine]; |
| 478 | (*S)-2,2-Difluoro-2′-(5-fluoropyridin-2-yl)-3′-(1H-pyrazolo[3,4-b]pyridin-4-y])- |
| | 5′H,7′H-spiro[cyclopropane-1,6′-pyrazolo[5,1-b][1,3]oxazine]; |
| 479 | (*R)-2,2-Difluoro-2′-(5-fluoropyridin-2-yl)-3′-(1H-pyrazolo[3,4-b]pyridin-4- |
| | yl)-5′H,7′H-spiro[cyclopropane-1,6′-pyrazolo[5,1-b][1,3]oxazine]; |
| 480 | (5a *R,6a*S)-3-(5-Fluoro-1H-pyrazolo[3,4-b]pyridin-4-yl)-2-(5-fluoropyridin-2- |
| | yl)-5a,6,6a,7-tetrahydro-5H-cyclopropa[e]pyrazolo[5,1-b][1,3]oxazepine; |
| 481 | (5a*S,6a*R)-3-(5-Fluoro-1H-pyrazolo[3,4-b]pyridin-4-yl)-2-(5-fluoropyridin-2- |
| | yl)-5a,6,6a,7-tetrahydro-5H-cyclopropa[e]pyrazolo[5,1-b][1,3]oxazepane; |
| 482 | (5a*R,6a*S)-3-(5-Fluoro-6-methyl-1H-pyrazolo[3,4-b]pyridin-4-y])-2-(5- |
| | fluoropyridin-2-yl)-5a,6,6a,7-tetrahydro-5H-cyclopropa[e]pyrazolo[5,1- |
| | b][1,3]oxazepine; |
| 483 | (5a*S,6a*R)-3-(5-Fluoro-6-methyl-1H-pyrazolo[3,4-b]pyridin-4-yl)-2-(5- |
| | fluoropyridin-2-yl)-5a,6,6a,7-tetrahydro-5H-cyclopropa[e]pyrazolo[5,1- |
| | b][1,3]oxazepine; |
| 484 | 2-(5-Fluoropyridin-2-yl)-7-methyl-3-(1H-pyrazolo[3,4-b]pyridin-4-yl)-7,8- |
| | dihydro-4H,6H-pyrazolo[5,1-c][1,4]oxazepin-7-ol; |
| 485 | (*S)-2-(5-Fluoropyridin-2-yl)-7-methoxy-7-methyl-3-(1H-pyrazolo[3,4- |
| | b]pyridin-4-yl)-7,8-dihydro-4H,6H-pyrazolo[5,1-c][1,4]oxazepane; and |
| 486 | (*R)-2-(5-Fluoropyridin-2-yl)-7-methoxy-7-methyl-3-(1H-pyrazolo[3,4- |
| | b]pyridin-4-yl)-7,8-dihydro-4H,6H-pyrazolo[5,1-c][1,4]oxazepine; |

and pharmaceutically acceptable salts, isotopes, N-oxides, solvates, and stereoisomers thereof.

A further embodiment of the current invention is a compound selected from the group consisting of:
2-(5-Fluoropyridin-2-yl)-6,6-dimethyl-3-(1H-pyrazolo[3,4-b]pyridin-4-yl)-6,7-dihydro-4H-pyrazolo[5,1-c][1,4]oxazine;
2-(5-Fluoropyridin-2-yl)-6,6-bis(methyl-d₃)-3-(1H-pyrazolo[3,4-b]pyridin-4-yl)-6,7-dihydro-4H-pyrazolo[5,1-c][1,4]oxazine;
5-Fluoro-4-(2-(5-fluoropyridin-2-yl)-5,6-dihydro-4H-pyrrolo[1,2-b]pyrazol-3-yl)-6-methyl-1H-pyrazolo[3,4-b]pyridine;
(*S)-4-(6-Fluoro-2-(5-fluoropyridin-2-yl)-6-(methoxymethyl)-4,5,6,7-tetrahydropyrazolo[1,5-a]pyridin-3-yl)-6-methyl-1H-pyrazolo[3,4-b]pyridine;
N-(4-(2-(5-Fluoropyridin-2-yl)-6,6-dimethyl-6,7-dihydro-4H-pyrazolo[5,1-c][1,4]oxazin-3-yl)pyridin-2-yl)propionamide;
2-(4-Fluorophenyl)-6,6-dimethyl-3-(6-methyl-1H-pyrazolo[3,4-b]pyridin-4-yl)-6,7-dihydro-5H-pyrazolo[5,1-b][1,3]oxazine; and
(4aS,5aS)-2-(5-Fluoropyridin-2-yl)-3-(1H-pyrazolo[3,4-b]pyridin-4-yl)-4,4a,5,5a-tetrahydrocyclopropa[4,5]pyrrolo[1,2-b]pyrazole;
   and pharmaceutically acceptable salts, isotopes, N-oxides, solvates, and stereoisomers thereof.

An additional embodiment of the invention is a compound of Formula (I) having the Formula (IA):

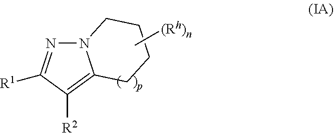

wherein
R¹ is selected from the group consisting of: 4-fluorophenyl, 5-fluoropyridin-2-yl, 3,5-difluoropyridin-2-yl,

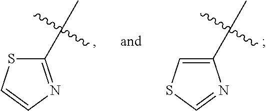 [SMILES: *c1nccs1 ; *c1cscn1]

R² is selected from the group consisting of:

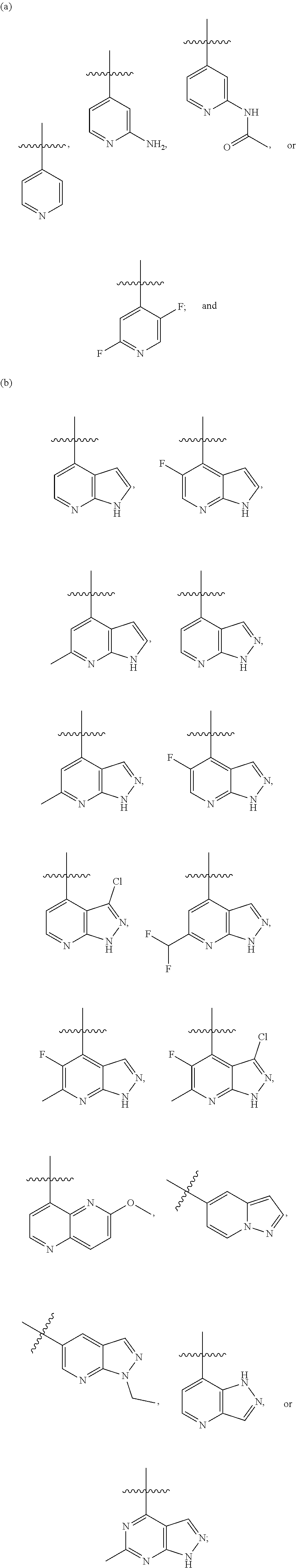

R^{h} is independently selected from the group consisting of: H, F, OH, CH₃, CD₃, CH₂F, CD₂F, CH₂OCH₃, CH₂OCD₃, CD₂₀CD₃, CH₂CH₂OCH₃, CH₂OCHF₂, CH₂OCF₃, CN, and

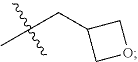 [SMILES: *CC1COC1]

n is 1, 2, or 3; and
p is 0 or 1.

An additional embodiment of the invention is a compound of Formula (I) having the Formula (IB):

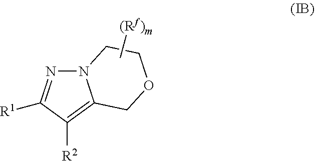

wherein
R¹ is selected from the group consisting of:

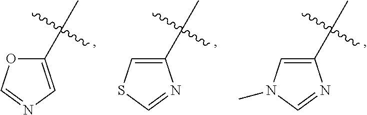

4-chlorophenyl, 4-fluorophenyl, 4-chloro-3-fluoro-phenyl, 5-fluoropyridin-2-yl, 5-chloropyridin-2-yl, 3-chloropyridin-4-yl, 5-fluoropyridin-3-yl, 3,5-difluoropyridin-2-yl, 3,5-difluoropyridin-4-yl, 5-fluoro-6-methyl-2-pyridyl, 6-methoxypyridin-2-yl, and 5-chloro-6-methylpyridin-2-yl;
R² is selected from the group consisting of:

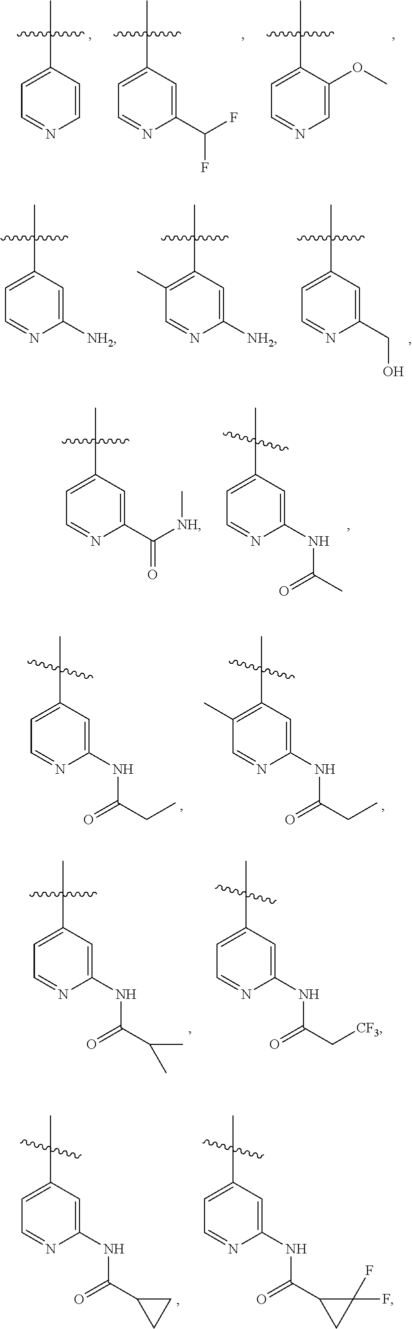

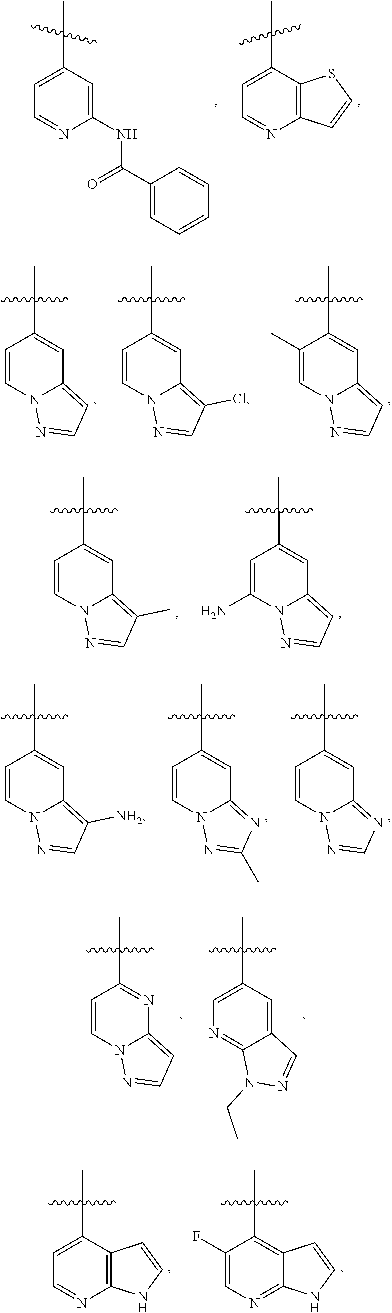

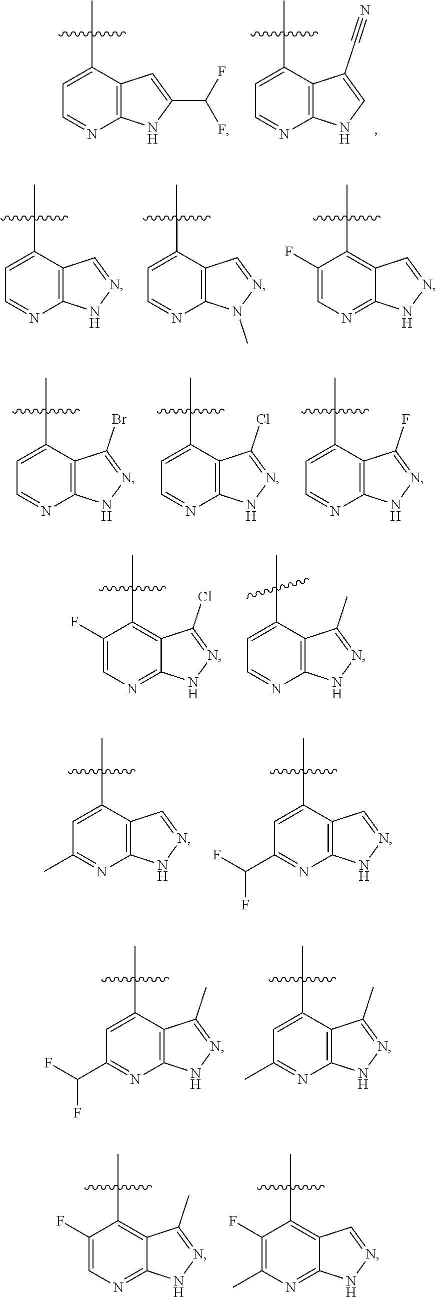

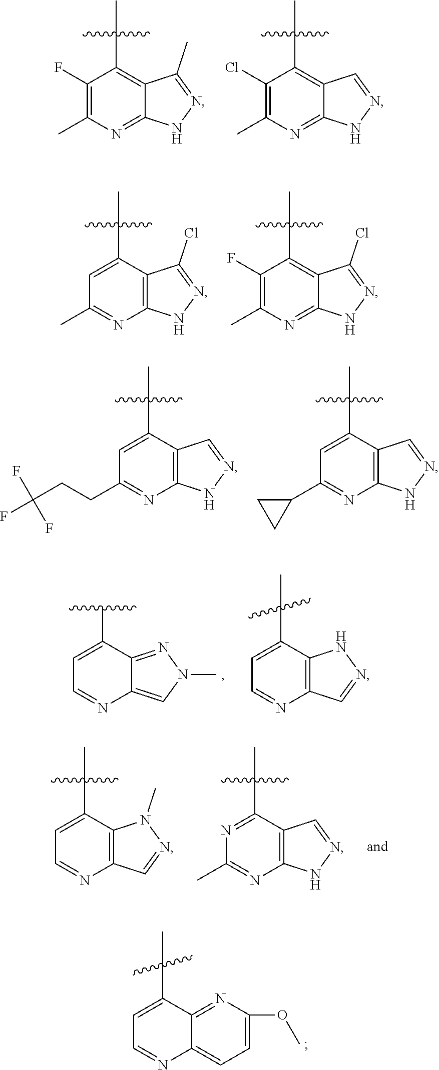

R^{f} is independently selected from the group consisting of: H, D, OH, CH₃, CD₃, CH₂CH₃, CH(CH₃)₂, CH₂F, CF₃, OCH₃, cyclopropyl, cyclobutyl, and two R^{f} members come together to form cyclopropyl wherein the cyclopropyl is optionally substituted with two F members; and
m is 1, 2, 3 or 4.

An additional embodiment of the invention is a compound of Formula (I) having the Formula (IC):

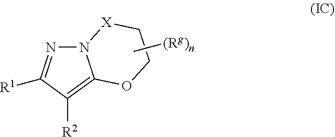

wherein
R¹ is selected from the group consisting of: 4-fluorophenyl, 5-fluoropyridin-2-yl, and 3,5-difluoropyridin-2-yl;
R² is selected from the group consisting of:

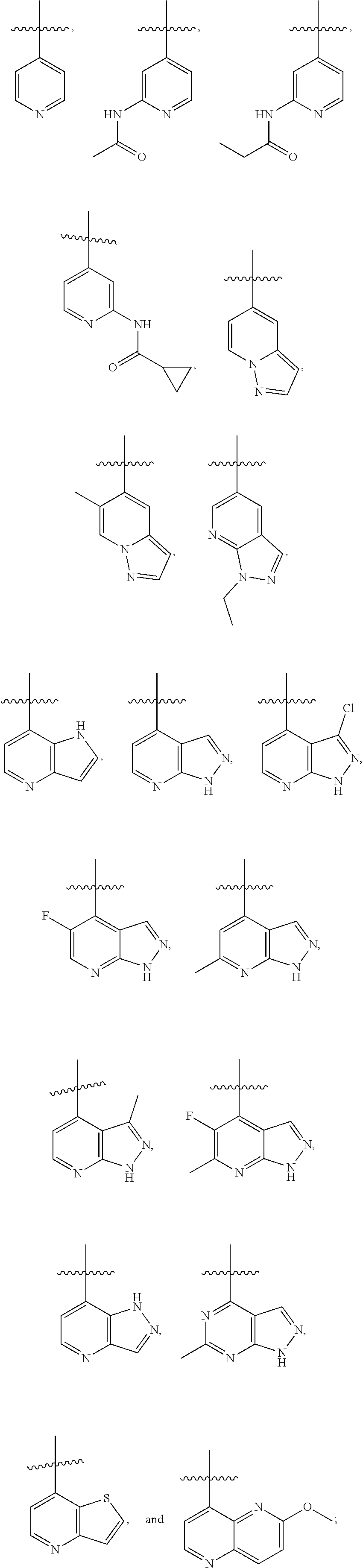

R^{g} is independently selected from the group consisting of: H, F, and CH₃;
X is selected from the group consisting of: a bond, CH₂, CH(CH₃), and CH₂CH₂; and
n is 1 or 2.

An additional embodiment of the invention is a compound of Formula (I) having the Formula (ID):

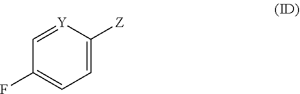

wherein
Y is CH or N; and
Z is selected from the group consisting of:

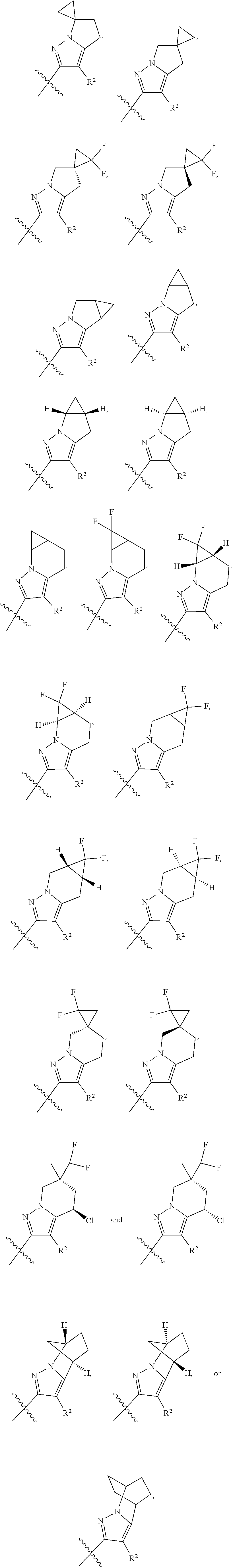

R² is

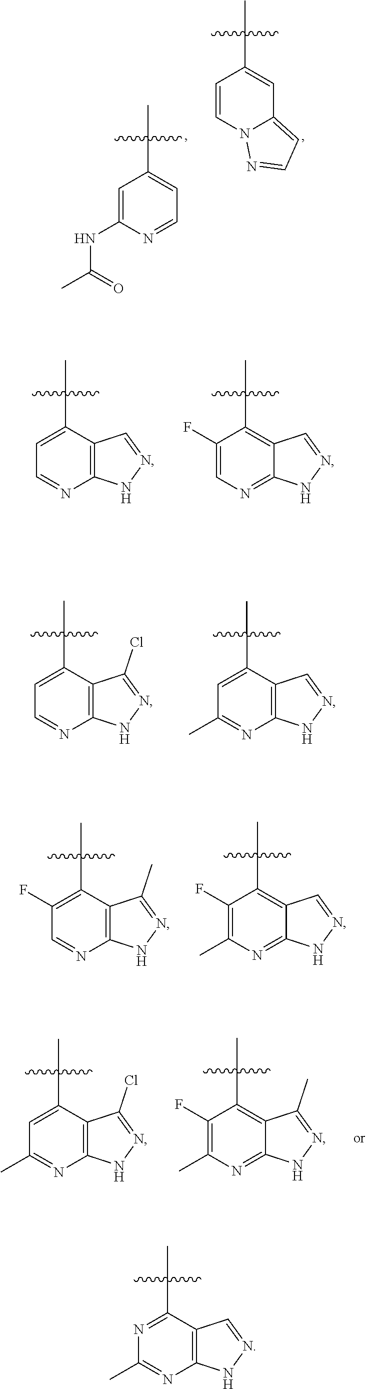

As used herein, the term “compound of the invention” includes all compounds encompassed by Formula (I) such as the species embodied in Formula (I-1), Formula (IA), Formula (IB), Formula (IC), and Formula (ID) or a combination thereof.

An additional embodiment of the invention is a pharmaceutical composition comprising:
(A) a therapeutically effective amount of at least one compound selected from compounds of Formula (I)

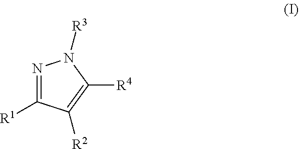

wherein
R¹ is selected from the group consisting of:
(a) phenyl substituted with one or two halo members;
(b) 5-fluoro-2-pyridyl optionally substituted with halo or C₁₋₃alkyl, 5-fluoro-3-pyridyl, 5-chloropyridin-2-yl, 3-chloropyridin-4-yl, 6-methoxypyridin-2-yl, 5-chloro-6-methylpyridin-2-yl, or 3,5-difluoropyridin-4-yl; and
(c) oxazol-5-yl, thiazol-2-yl, thiazol-4-yl, or 1-methyl-1H-imidazol-4-yl;
R² is selected from the group consisting of:
(d) 4-pyridyl optionally substituted with one member selected from the group consisting of: halo, C₁₋₃haloalkyl, CH₂OH, OC₁₋₃alkyl, (C═O)—NHCH₃, NH₂, NH—(C═O)C₁₋₃alkyl, NH—(C═O)C₁₋₃haloalkyl, NH—(C═O)phenyl, NH—(C═O)cyclopropyl, and NH—(C═O)cyclopropyl wherein the cyclopropyl is substituted with one or two halo; 2,5-difluoro-4-pyridyl;

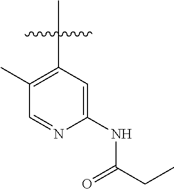 [SMILES: *c1cc(NC(=O)CC)ncc1C]

or 5-methylpyridin-2-amine;
(e) fused heteroaryl selected from the group consisting of: thieno[3,2-b]pyridinyl; pyrazolo[1,5-a]pyridin-5-yl optionally substituted with halo, C₁₋₃alkyl, or NH₂; 1H-pyrrolo[3,2-b]pyridinyl; 1H-pyrrolo[2,3-b]pyridinyl optionally substituted with one two or three members each independently selected from the group consisting of: halo, C₁₋₃alkyl, C₁₋₃haloalkyl, and CN; pyrazolo[1,5-a]pyrimidin-5-yl; [1,2,4]triazolo[1,5-a]pyridin-7-yl optionally substituted with C₁₋₃alkyl; 1H-pyrazolo[4,3-b]pyridin-7-yl optionally substituted with C₁₋₃alkyl; 1H-pyrazolo[3,4-b]pyridin-5-yl optionally substituted with C₁₋₃alkyl; 1H-pyrazolo[3,4-b]pyridinyl optionally substituted with one, two or three members each independently selected from the group consisting of: halo, C₁₋₃alkyl, C₁₋₃haloalkyl, and cyclopropyl; 2-methyl-2H-pyrazolo[4,3-b]pyridin-7-yl; 1-methyl-1H-pyrazolo[4,3-b]pyridin-7-yl; 2-methylpyrazolo[3,4-b]pyridin-4-yl; or 1H-pyrazolo[3,4-d]pyrimidin-4-yl optionally substituted with C₁₋₃alkyl; and
(f) 1,5-naphthyridin-4-yl optionally substituted with halo or OC₁₋₃alkyl;
R³ and R⁴ come together to form a group selected from the group consisting of:

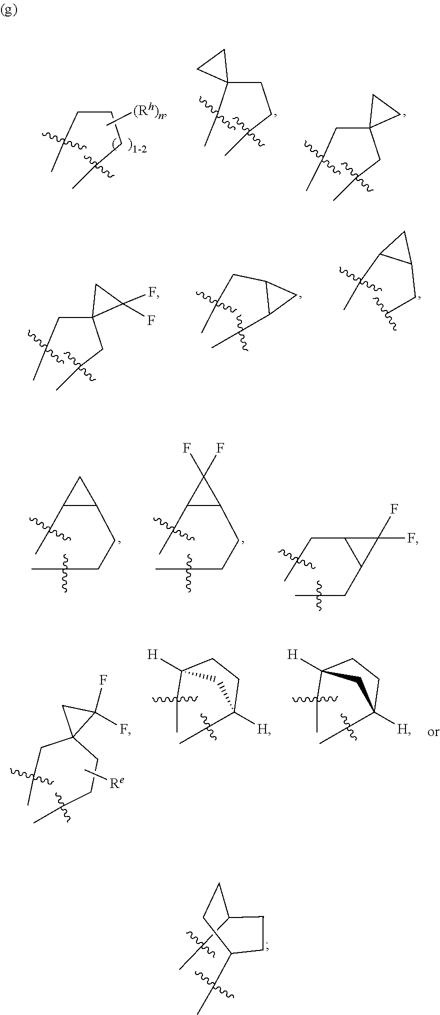

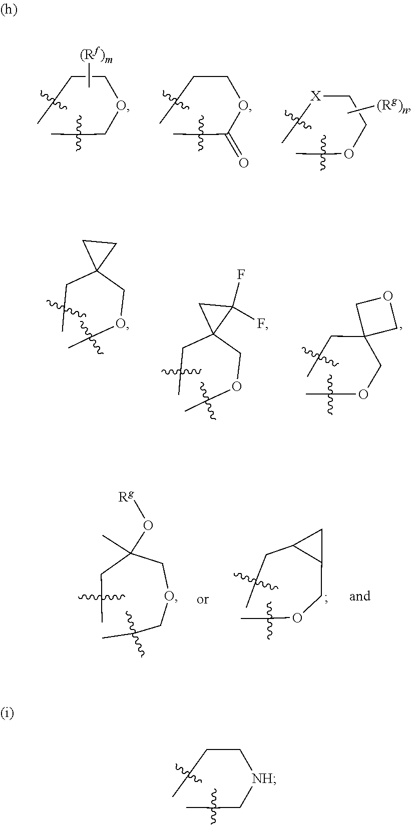

R^{f} is independently selected from the group consisting of: H, C₁₋₃alkyl, C₁₋₃haloalkyl, cyclopropyl, cyclobutyl, and two R^{f} members come together to form a C₃₋₆cycloalkyl wherein the C₃₋₆cycloalkyl is optionally substituted with one or two halo members;
R^{g} is H, halo, or C₁₋₃alkyl;
R^{h} is independently selected from the group consisting of H, halo, OH, C₁₋₃alkyl, CH₂OCH₃, CH₂CH₂OCH₃, C₁₋₃haloalkyl, CH₂OCHF₂, CH₂OCF₃, CN, and

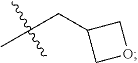 [SMILES: *CC1COC1]

X is selected from the group consisting of: a bond, CH₂, CH(CH₃), and CH₂CH₂;
m is 1, 2, 3 or 4; and
n is 1, 2, or 3;
and pharmaceutically acceptable salts, isotopes, N-oxides, solvates, and stereoisomers of compounds of Formula (I);
and (B) at least one pharmaceutically acceptable excipient.

An additional embodiment of the invention is a pharmaceutical composition comprising a therapeutically effective amount of at least one compound in Table 1, as well as and pharmaceutically acceptable salts, isotopes, N-oxides, solvates, and stereoisomers of compounds of Table 1, pharmaceutically acceptable prodrugs of compounds of Table 1, and pharmaceutically active metabolites of Table 1; and at least one pharmaceutically acceptable excipient.

An additional embodiment of the invention is a pharmaceutical composition comprising a therapeutically effective amount of at least one compound of Formula (IA), as well as pharmaceutically acceptable salts, N-oxides or solvates of compounds of Formula (IA), pharmaceutically acceptable prodrugs of compounds of Formula (IA), and pharmaceutically active metabolites of Formula (IA); and at least one pharmaceutically acceptable excipient.

An additional embodiment of the invention is a pharmaceutical composition comprising a therapeutically effective amount of at least one compound of Formula (IB), as well as pharmaceutically acceptable salts, N-oxides or solvates of compounds of Formula (IB), pharmaceutically acceptable prodrugs of compounds of Formula (IB), and pharmaceutically active metabolites of Formula (IB); and at least one pharmaceutically acceptable excipient.

An additional embodiment of the invention is a pharmaceutical composition comprising a therapeutically effective amount of at least one compound of Formula (IC), as well as pharmaceutically acceptable salts, N-oxides or solvates of compounds of Formula (IC), pharmaceutically acceptable prodrugs of compounds of Formula (IC), and pharmaceutically active metabolites of Formula (IC); and at least one pharmaceutically acceptable excipient.

An additional embodiment of the invention is a pharmaceutical composition comprising a therapeutically effective amount of at least one compound of Formula (ID), as well as pharmaceutically acceptable salts, N-oxides or solvates of compounds of Formula (ID), pharmaceutically acceptable prodrugs of compounds of Formula (ID), and pharmaceutically active metabolites of Formula (ID); and at least one pharmaceutically acceptable excipient.

Also within the scope of the invention are enantiomers and diastereomers of the compounds of Formula (I) (as well as Formula (I-1), Formula (IA), Formula (IB), Formula (IC) and Formula (ID)) Also within the scope of the invention are the pharmaceutically acceptable salts, N-oxides or solvates of the compounds of Formula (I) (as well as Formula (I-1), Formula (IA), Formula (IB), Formula (IC) and Formula (ID)). Also within the scope of the invention are the pharmaceutically acceptable prodrugs of compounds of Formula (I) (as well as Formula (I-1), Formula (IA), Formula (IB), Formula (IC) and Formula (ID)), and pharmaceutically active metabolites of the compounds of Formula (I) (as well as Formula (I-1), Formula (IA), Formula (IB), Formula (IC) and Formula (ID)).

Also within the scope of the invention are isotopic variations of compounds of Formula (I) (as well as Formula (I-1), Formula (IA), Formula (IB), Formula (IC) and Formula (ID)), such as, e.g., deuterated compounds of Formula (I). Also within the scope of the invention are the pharmaceutically acceptable salts, N-oxides or solvates of the isotopic variations of the compounds of Formula (I) (as well as Formula (I-1), Formula (IA), Formula (IB), Formula (IC) and Formula (ID)). Also within the scope of the invention are the pharmaceutically acceptable prodrugs of the isotopic variations of the compounds of Formula (I) (Formula (I) (as well as Formula (I-1), Formula (IA), Formula (IB), Formula (IC) and Formula (ID)), and pharmaceutically active metabolites of the isotopic variations of the compounds of Formula (I) (as well as Formula (I-1), Formula (IA), Formula (IB), Formula (IC) and Formula (ID)).

An additional embodiment of the invention is a method of treating a subject suffering from or diagnosed with a disease, disorder, or condition mediated by protein kinase CSNK1D activity, comprising administering to a subject in need of such treatment a therapeutically effective amount of at least one compound selected from compounds of Formula (I):

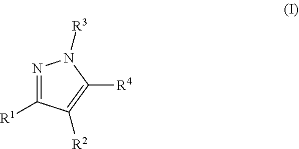

wherein
R¹ is selected from the group consisting of:
   (a) phenyl substituted with one or two halo members;
   (b) 5-fluoro-2-pyridyl optionally substituted with halo or C₁₋₃alkyl, 5-fluoro-3-pyridyl, 5-chloropyridin-2-yl, 3-chloropyridin-4-yl, 6-methoxypyridin-2-yl, 5-chloro-6-methylpyridin-2-yl, or 3,5-difluoropyridin-4-yl; and
   (c) oxazol-5-yl, thiazol-2-yl, thiazol-4-yl, or 1-methyl-1H-imidazol-4-yl;
R² is selected from the group consisting of:
   (d) 4-pyridyl optionally substituted with one member selected from the group consisting of: halo, C₁₋₃haloalkyl, CH₂OH, OC₁₋₃alkyl, (C═O)—NHCH₃, NH₂, NH—(C═O)C₁₋₃alkyl, NH—(C═O)C₁₋₃haloalkyl, NH—(C═O)phenyl, NH—(C═O)cyclopropyl, and NH—(C═O)cyclopropyl wherein the cyclopropyl is substituted with one or two halo; 2,5-difluoro-4-pyridyl;

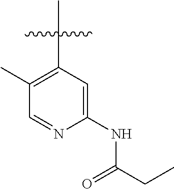 [SMILES: *c1cc(NC(=O)CC)ncc1C]

or 5-methylpyridin-2-amine;
(e) fused heteroaryl selected from the group consisting of: thieno[3,2-b]pyridinyl; pyrazolo[1,5-a]pyridin-5-yl optionally substituted with halo, C₁₋₃alkyl, or NH₂; 1H-pyrrolo[3,2-b]pyridinyl; 1H-pyrrolo[2,3-b]pyridinyl optionally substituted with one two or three members each independently selected from the group consisting of: halo, C₁₋₃alkyl, C₁₋₃haloalkyl, and CN; pyrazolo[1,5-a]pyrimidin-5-yl; [1,2,4]triazolo[1,5-a]pyridin-7-yl optionally substituted with C₁₋₃alkyl; 1H-pyrazolo[4,3-b]pyridin-7-yl optionally substituted with C₁₋₃alkyl; 1H-pyrazolo[3,4-b]pyridin-5-yl optionally substituted with C₁₋₃alkyl; 1H-pyrazolo[3,4-b]pyridinyl optionally substituted with one, two or three members each independently selected from the group consisting of: halo, C₁₋₃alkyl, C₁₋₃haloalkyl, and cyclopropyl; 2-methyl-2H-pyrazolo[4,3-b]pyridin-7-yl; 1-methyl-1H-pyrazolo[4,3-b]pyridin-7-yl; 2-methylpyrazolo[3,4-b]pyridin-4-yl; or 1H-pyrazolo[3,4-d]pyrimidin-4-yl optionally substituted with C₁₋₃alkyl; and
(f) 1,5-naphthyridin-4-yl optionally substituted with halo or OC₁₋₃alkyl;
R³ and R⁴ come together to form a group selected from the group consisting of:

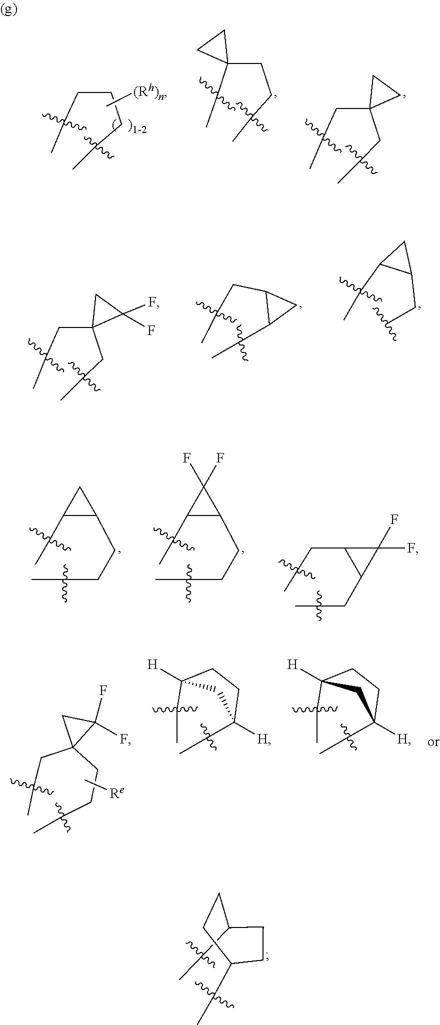

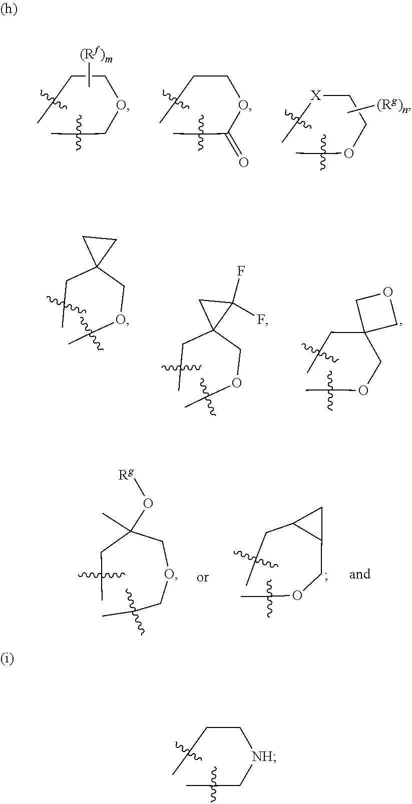

R^{f} is independently selected from the group consisting of: H, C₁₋₃alkyl, C₁₋₃haloalkyl, cyclopropyl, cyclobutyl, and two R^{f} members come together to form a C₃₋₆cycloalkyl wherein the C₃₋₆cycloalkyl is optionally substituted with one or two halo members;
R^{g} is H, halo, or C₁₋₃alkyl;
R^{h} is independently selected from the group consisting of H, halo, OH, C₁₋₃alkyl, CH₂OCH₃, CH₂CH₂OCH₃, C₁₋₃haloalkyl, CH₂OCHF₂, CH₂OCF₃, CN, and

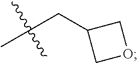 [SMILES: *CC1COC1]

X is selected from the group consisting of a bond, CH₂, CH(CH₃), and CH₂CH₂;
m is 1, 2, 3 or 4; and
n is 1, 2, or 3;
and pharmaceutically acceptable salts, isotopes, N-oxides, solvates, and stereoisomers thereof, to a subject in need thereof.

An additional embodiment of the invention is a method of treating a subject suffering from or diagnosed with a disease, disorder, or condition mediated by protein kinase CSNK1D receptor activity, comprising administering to a subject in need of such treatment a therapeutically effective amount of at least one compound selected from compounds of Formula (I) (as well as Formula (I-1), Formula (IA), Formula (IB), Formula (IC) and Formula (ID)), enantiomers and diastereomers of the compounds of (Formula (I) (as well as Formula (I-1), Formula (IA), Formula (IB), Formula (IC) and Formula (ID)), isotopic variations of the compounds of Formula (I) (Formula (I) (as well as Formula (I-1), Formula (IA), Formula (IB), Formula (IC) and Formula (ID)), and pharmaceutically acceptable salts of all of the foregoing.

Exemplary compounds useful in methods of the invention will now be described by reference to the illustrative synthetic schemes for their general preparation below and the specific examples that follow. Artisans will recognize that, to obtain the various compounds herein, starting materials may be suitably selected so that the ultimately desired substituents will be carried through the reaction scheme with or without protection as appropriate to yield the desired product. Alternatively, it may be necessary or desirable to employ, in the place of the ultimately desired substituent, a suitable group that may be carried through the reaction scheme and replaced as appropriate with the desired substituent. Unless otherwise specified, the variables are as defined above in reference to Formula (I). Reactions may be performed between the melting point and the reflux temperature of the solvent, and preferably between 0° C. and the reflux temperature of the solvent. Reactions may be heated employing conventional heating or microwave heating. Reactions may also be conducted in sealed pressure vessels above the normal reflux temperature of the solvent.

Abbreviations and acronyms used herein include the following:

TABLE 2

| Acronym | Term |
|---|---|
| Å | Angstrom |
| μL | Microliter |
| μmol, μm | micromole |
| ACN, MeCN | Acetonitrile |
| AcOH, HOAc | Acetic acid |
| Aq, or Aq. | Aqueous |
| atm | Atmosphere |
| BPin | Bis(pinacolato)diboron |
| [BMIM]BF4 | 1-Butyl-3-methylimidazolium tetrafluoroborate |
| br | Broad |
| ° C. | Celsius |
| Calcd. | Calculated |
| CataCXium ® A Pd | Mesylate[(di(1-adamanty])-n-butylphosphine)-2-(2′-amino-1,1′- |
| G4 | biphenyl)]palladium(II) |
| CataCXium ® A-Pd- | Chloro[(di(1-adamanty])-N-butylphosphine)-2-(2- |
| G2 | aminobiphenyl)]palladium(II) |
| CataCXium ® A Pd | Mesylate[(di(1-adamanty])-n-butylphosphine)-2-(2′-amino-1,1′- |
| G3 | biphenyl)]palladium(II) |
| CbzCl | Benzyl chloroformate |
| Celite ® | Diatomaceous Earth |
| Conc. | concentrated |
| DAST | Diethylaminosulfur trifluoride |
| DBU | 1,8-Diazabicyclo[5.4.0]undec-7-ene |
| DCM | dichloromethane |
| DIBAL-H | Diisobutyl aluminum hydride |
| DMA, DMAc | Dimethylacetamide |
| DMAP | 4-dimethylaminopyridine |
| DME | dimethoxyethane |
| DMF | dimethylformamide |
| DMSO | Dimethylsulfoxide |
| Dowtherm ™ A | Heat Transfer Fluid; eutectic mixture of biphenyl and diphenyl oxide |
| DPPA | Diphenylphosphoryl azide |
| DPPF | 1,1′-bis(diphenylphosphino)ferrocene |
| ESI | Electrospray ionization |
| Ether, Et₂O | Diethyl ether |
| EtOAc, or EA | Ethyl Acetate |
| EtOH | Ethanol |
| eq, equiv | Equivalents |
| FCC | Normal-phase silica gel chromatography |
| g | Grams |
| h, hr, hrs | Hours |
| HATU | 1-[bis(dimethylamino)methylene]-1H-1,2,3-triazolo[4,5-b]pyridinium |
| | 3-oxid hexafluorophosphate |
| Hex | hexanes |
| HMPA | Hexamethylphosphoramide |
| HPLC | High-pressure liquid chromatography |
| Hunig's base | N,N-Diisopropylethylamine |
| Hz | Hertz |
| IPA | Isopropyl alcohol |
| t-BuOK, tBuOK | Potassium tert-Butoxide |
| LCMS | Liquid chromatography and mass spectrometry |
| LiHMDS or | lithium bis(trimethylsilyl)amide |
| LHMDS | |
| M | Molar |
| m/z | Mass to charge ratio |
| Me | methyl |
| MeOH | Methanol |
| mg | Milligrams |
| MHz | Megahertz |
| min | Minute |
| mL | Milliliter |
| mm | millimeter |
| mM | Millimolar |
| mmol | Millimole |
| mol | moles |
| MS | Mass spectrometry |
| MsCl | Methanesulfonyl chloride |
| Ms | Methanesulfonyl |
| MTBE or TBME | tert-butyl methyl ether |
| N | Normal |
| NBS | N-Bromosuccinimide |
| NFSI | N-Fluorobenzenesulfonimide |
| nm | nanometer |
| NMP | N-Methyl-2-pyrrolidone |
| NMR | Nuclear magnetic resonance |
| Pd/C | Palladium on carbon |
| Pd(dppf)Cl₂•DCM | [1,1′-Bis(diphenylphosphino)ferrocene]dichloropalladium(II), complex |
| | with dichloromethane |
| PdCl₂(Cy*Phine)₂ | Dichlorobis(dicyclohexyl(2′,4′,6′-triisopropyl-[1,1′:3′,1″-terphenyl]-2- |
| | yl)phosphane)palladium(II) |
| Pd(amphos)Cl₂ | Bis(di-tert-butyl(4- |
| | dimethylaminophenyl)phosphine)dichloropalladium(II) |
| PE | Petroleum ether |
| ppm | Parts per million |
| psi | Pounds per square inch |
| PyBroP ® | bromotripyrrolidinophosphonium hexafluorophosphate |
| Rf | Retention factor |
| Rochelle's salt | Potassium sodium tartrate |
| RP | Reverse Phase |
| Rₜ | Retention time |
| rt, RT, or r.t. | Room temperature |
| sat, sat. | Saturated |
| SEMCI | 2-(Trimethylsilyl)ethoxymethyl chloride |
| SFC | Supercritical Fluid Chromatography |
| T3P ® | 2,4,6-tripropyl-1,3,5,2,4,6-trioxatriphosphorinane-2,4,6-trioxide |
| TBSCl | tert-Butyldimethylsilyl chloride |
| TBAF | Tetra-n-butylammonium fluoride |
| t-BuOH | tert-Butyl alcohol |
| TEA, Et₃N | triethylamine |
| TFA | trifluoroacetic acid |
| THF | tetrahydrofuran |
| TLC | Thin layer chromatography |
| TsCl, TosCl | 4-Toluenesulfonyl chloride |
| TsOH | p-Toluenesulfonic acid |
| V, or volumes | Volume in milliliters of solvent per gram of substrate |
| wt. | Weight |
| XPhos Pd G3 | (2-Dicyclohexylphosphino-2′,4′,6′-triisopropyl-1,1′-bipheny1)[2-(2′- |
| | amino-1,1′-bipheny1)]palladium(II) methanesulfonate |
| XtalFluor-E ® | (Diethylamino)difluorosulfonium tetrafluoroborate |

### Preparative Examples

Exemplary compounds useful in methods of the invention will now be described by reference to the illustrative synthetic schemes for their general preparation below and the specific examples to follow

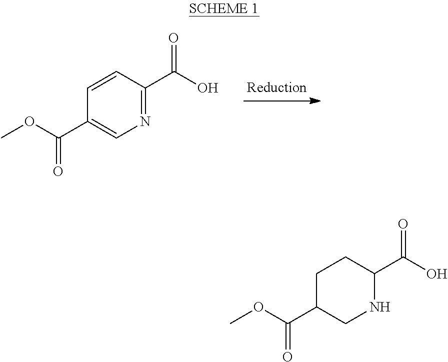

According to SCHEME 1, commercially available or synthetically accessible 5-(methoxycarbonyl)piperidine-2-carboxylic acid is prepared starting from 5-(methoxycarbonyl)picolinic acid by treatment with a hydrogenation catalyst such as palladium on carbon (Pd/C) and the like, in a suitable solvent mixture such as glacial acetic acid (AcOH)/methanol (MeOH) and the like, under 2 MPa of hydrogen (H₂)

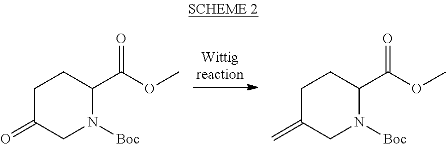

According to SCHEME 2, 1-tert-butyl 2-methyl 5-methylenepiperidine-1,2-dicarboxylate is prepared in a Wittig reaction between 1-tert-butyl 2-methyl 5-oxopiperidine-1,2-dicarboxylate and methyltriphenylphosphonium bromide, in the presence of a suitable strong base such as KHMDS, in a solvent such as toluene

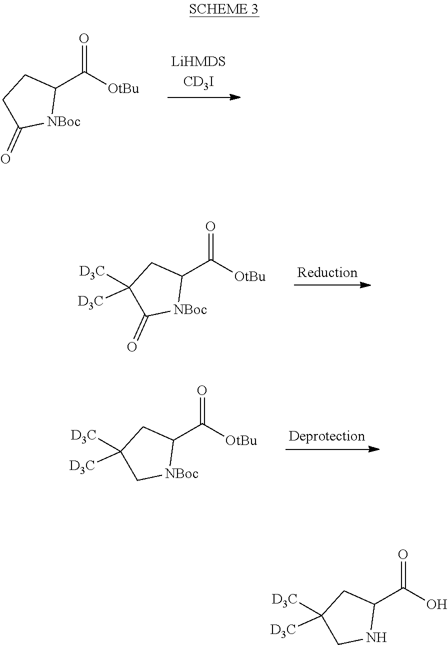 [SMILES: CC(C)(C)OC(=O)C1CCC(=O)N1C(=O)OC(C)(C)C ; [2H]C([2H])([2H])C1(C([2H])([2H])[2H])CC(C(=O)OC(C)(C)C)N(C(=O)OC(C)(C)C)C1=O ; [2H]C([2H])([2H])C1(C([2H])([2H])[2H])CC(C(=O)OC(C)(C)C)N(C(=O)OC(C)(C)C)C1 ; [2H]C([2H])([2H])C1(C([2H])([2H])[2H])CNC(C(=O)O)C1]

According to SCHEME 3, di-tert-butyl 4,4-bis(methyl-d₃)-5-oxopyrrolidine-1,2-dicarboxylate is prepared from (S)-di-tert-butyl 5-oxopyrrolidine-1,2-dicarboxylate by deprotonation with a strong base such as LiHMDS and the like, followed by treatment with CD₃I, in a suitable solvent such as THF and the like. Di-tert-butyl 4,4-bis(methyl-d₃)pyrrolidine-1,2-dicarboxylate is prepared from di-tert-butyl 4,4-bis(methyl-d₃)-5-oxopyrrolidine-1,2-dicarboxylate using a reducing agent such as BH₃·THF and the like, in a suitable solvent such as THF and the like. 4,4-bis(Methyl-d₃)pyrrolidine-2-carboxylic acid hydrochloride is prepared by acid-mediated deprotection of di-tert-butyl 4,4-bis(methyl-d₃)pyrrolidine-1,2-dicarboxylate, using conditions known to one skilled in the art

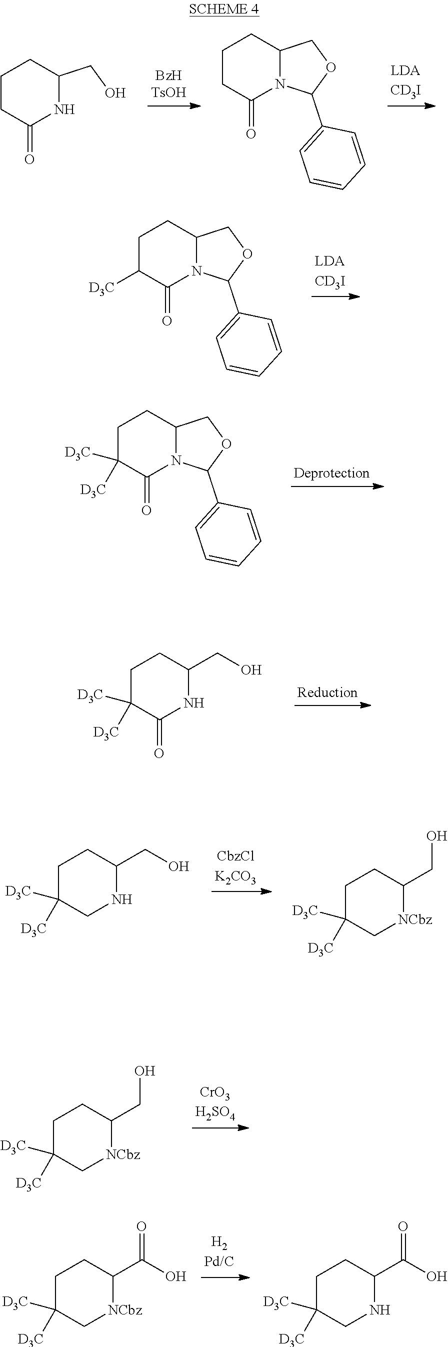

According to SCHEME 4, 6-(hydroxymethyl)piperidin-2-one is reacted with benzaldehyde, in the presence of an acid catalyst such as TsOH and the like, in a suitable solvent such as toluene and the like, to provide 3-phenyltetrahydro-1H-oxazolo[3,4-a]pyridin-5(3H)-one. 3-Phenyltetrahydro-1H-oxazolo[3,4-a]pyridin-5(3H)-one is deprotonated using a strong base such as LDA and the like, then treated with CD₃I, in a suitable solvent such as THF and the like, to provide 3-phenyl-6-(methyl-d₃)tetrahydro-1H-oxazolo[3,4-a]pyridin-5(3H)-one. 3-Phenyl-6-(methyl-d₃)tetrahydro-1H-oxazolo[3,4-a]pyridin-5(3H)-one is deprotonated using a strong base such as LDA and the like, then treated with an alkylating agent such as CD₃I, in a suitable solvent such as THF and the like, to provide 3-phenyl-6,6-bis-(methyl-d₃)tetrahydro-1H-oxazolo[3,4-a]pyridin-5(3H)-one. 3-Phenyl-6,6-bis-(methyl-d₃)tetrahydro-1H-oxazolo[3,4-a]pyridin-5(3H)-one is deprotected using acidic deprotection conditions such as TFA/CH₂Cl, to provide 6-(hydroxymethyl)-3,3-bis(methyl-d₃)piperidin-2-one. 6-(Hydroxymethyl)-3,3-bis(methyl-d₃)piperidin-2-one is reduced using a suitable reducing agent such as LiAlH₄, and the like, in a solvent such as THF and the like, at temperatures ranging from 0° C. to 65° C., to provide (5,5-bis(methyl-d₃)piperidin-2-yl)methanol. (5,5-Bis(methyl-d₃)piperidin-2-yl)methanol is reacted with CbzCl in the presence of a base such as K₂CO₃ and the like, in a suitable solvent or mixture of solvents such as THF/H₂O and the like, to provide benzyl 2-(hydroxymethyl)-5,5-bis(methyl-d₃)piperidine-1-carboxylate. Oxidation of benzyl 2-(hydroxymethyl)-5,5-bis(methyl-d₃)piperidine-1-carboxylate is achieved using an oxidizing agent such as CrO₃/H₂SO₄ and the like, in a suitable solvent such as water and the like, to provide 1-((benzyloxy)carbonyl)-5,5-bis(methyl-d₃)piperidine-2-carboxylic acid. Hydrogenation of 1-((benzyloxy)carbonyl)-5,5-bis(methyl-d₃)piperidine-2-carboxylic acid is achieved employing a suitable palladium catalyst such as 10% Pd/C, and the like; in a solvent such as EtOAc, MeOH, and the like; under H₂ (15 psi), to afford 5,5-bis(methyl-d₃)piperidine-2-carboxylic acid

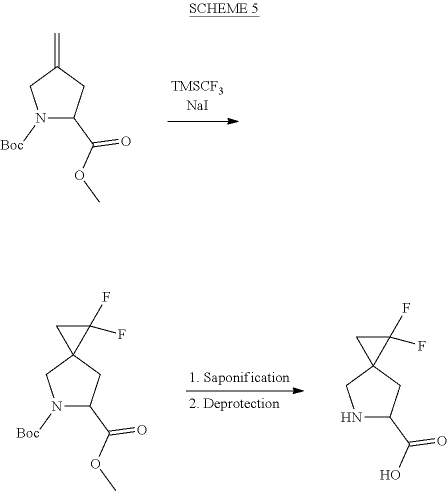 [SMILES: C=C1CC(C(=O)OC)N(C(=O)OC(C)(C)C)C1 ; COC(=O)C1CC2(CN1C(=O)OC(C)(C)C)CC2(F)F ; O=C(O)C1CC2(CN1)CC2(F)F]

According to SCHEME 5, (S)-1-tert-butyl 2-methyl 4-methylenepyrrolidine-1,2-dicarboxylate is reacted with trifluoromethyltrimethylsilane (known as Ruppert-Prakash reagent, TMSCF₃), sodium iodide, in a suitable solvent such as THF and the like, at a temperature ranging from rt to 70° C., for a period of 12 to 16 hrs, to provide (6S)-5-tert-butyl 6-methyl 1,1-difluoro-5-azaspiro[2.4]heptane-5,6-dicarboxylate. (6S)-1,1-Difluoro-5-azaspiro[2.4]heptane-6-carboxylic acid hydrochloride is prepared in two steps from (6S)-5-tert-butyl 6-methyl 1,1-difluoro-5-azaspiro[2.4]heptane-5,6-dicarboxylate. In a first step, (6S)-5-tert-butyl 6-methyl 1,1-difluoro-5-azaspiro[2.4]heptane-5,6-dicarboxylate is saponified using a suitable base such as lithium hydroxide monohydrate, aqueous sodium hydroxide (NaOH), and the like, in a suitable solvent such as EtOH, water, or a mixture thereof, at a temperature of 60° C. to 80° C., for a period of 1-6 h, to provide (6S)-5-(tert-butoxycarbonyl)-1,1-difluoro-5-azaspiro[2.4]heptane-6-carboxylic acid. (6S)-5-(tert-Butoxycarbonyl)-1,1-difluoro-5-azaspiro[2.4]heptane-6-carboxylic acid is deprotected with an acid such as TFA, HCl, and the like; in a suitable solvent such as DCM, and the like, at rt, to provide (6S)-1,1-difluoro-5-azaspiro[2.4]heptane-6-carboxylic acid.

In a similar fashion, 1,1-difluoro-5-azaspiro[2.5]octane-6-carboxylic acid is prepared from 1-tert-butyl 2-methyl 5-methylenepiperidine-1,2-dicarboxylate; employing methods as described above

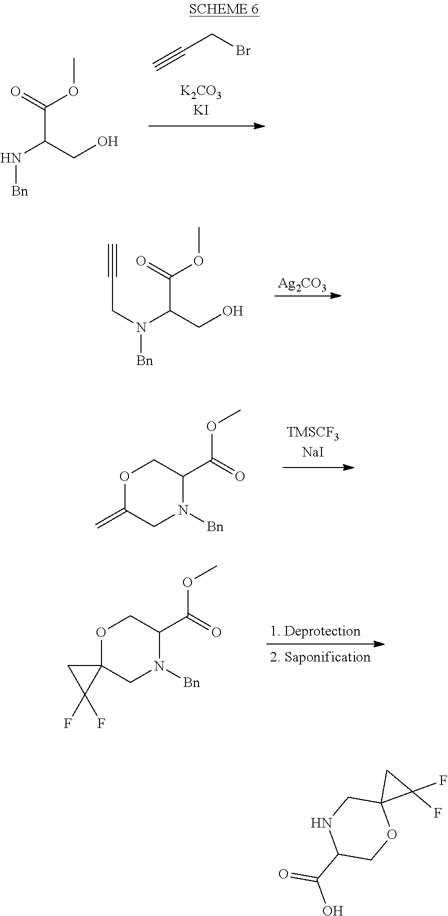

According to SCHEME 6, S_{N}2 alkylation of methyl 2-(benzylamino)-3-hydroxypropanoate; using a base such as K₂CO₃, Cs₂CO₃, sodium hydride (NaH), and the like; with or without an additive such as KI; a suitable solvent such as ACN, THF, DMF, DCM, N-methyl pyrrolidone (NMP), and the like; for a period of 4-16 h; affords methyl 2-(benzyl(prop-2-yn-1-yl)amino)-3-hydroxypropanoate. Cyclization of methyl 2-(benzyl(prop-2-yn-1-yl)amino)-3-hydroxypropanoate, using a catalyst such as Ag₂CO₃ and the like, in a suitable solvent such as toluene at the like, affords methyl 4-benzyl-6-methylenemorpholine-3-carboxylate. Difluorocylopronanation of methyl 4-benzyl-6-methylenemorpholine-3-carboxylate is achieved with trifluoromethyltrimethylsilane, employing methods previously described to afford methyl 7-benzyl-1, 1-difluoro-4-oxa-7-azaspiro[2.5]octane-6-carboxylate. Hydrogenolysis of methyl 7-benzyl-1, 1-difluoro-4-oxa-7-azaspiro[2.5]octane-6-carboxylate is achieved employing methods previously described to afford methyl 1, 1-difluoro-4-oxa-7-azaspiro [2.5] octane-6-carboxylate. Saponification of methyl 1, 1-difluoro-4-oxa-7-azaspiro [2.5] octane-6-carboxylate using conditions previously described affords 1,1-difluoro-4-oxa-7-azaspiro[2.5]octane-6-carboxylic acid

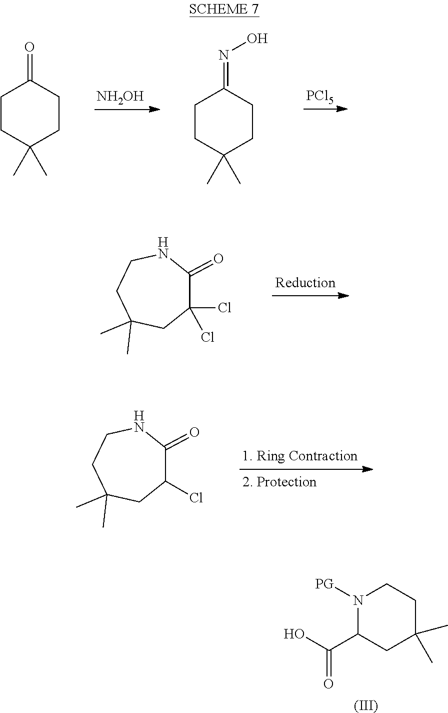

According to SCHEME 7, 1-((benzyloxy)carbonyl)-4,4-dimethylpiperidine-2-carboxylic acid is prepared starting from 4,4-dimethylcyclohexan-1-one. 4,4-Dimethylcyclohexan-1-one oxime is prepared from 4,4-dimethylcyclohexan-1-one by treatment with hydroxylamine hydrochloride, in the presence of a suitable base such as sodium carbonate (Na₂CO₃) and the like, in a suitable mixture of solvents such as ethanol (EtOH) and water (H₂O), at a temperature of 100° C. 3,3-Dichloro-5,5-dimethylazepan-2-one is prepared by the ring expansion of 4,4-dimethylcyclohexan-1-one oxime, using an electrophile such as phosphorus pentachloride (PCl₅) and the like, in a solvent such as xylenes and the like, at a temperature ranging from 35° C. to 90° C. 3-Chloro-5,5-dimethylazepan-2-one is prepared by the reduction of 3,3-dichloro-5,5-dimethylazepan-2-one, using a hydrogenation catalyst such as palladium on carbon (Pd/C) and the like, in a solvent such as glacial acetic acid (AcOH) and the like, under an atmosphere of 50 psi hydrogen (H₂). A compound of formula (III), where PG is a CBz protecting group, is prepared from 3-chloro-5,5-dimethylazepan-2-one using an inorganic base such as barium hydroxide octahydrate and the like, in a suitable solvent such as H₂O and the like, at a temperature of 115° C., followed by treatment with a protecting reagent such as benzyl chloroformate (CbzCl), in a suitable solvent such as THF and the like, at room temperature

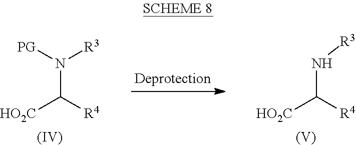

According to SCHEME 8, amino acids of formula (V), where R³ and R⁴ are as defined in Claim 1, are prepared from commercially available protected amino acids of formula (IV) (which includes a compound of formula (III)), where PG is a suitable nitrogen protecting group such as tert-butoxycarbonyl (BOC), benzyloxycarbonyl (Cbz), benzyl (Bn), and the like; using conditions known to one skilled in the art that are appropriate for the removal of the specific protecting group, such as TFA, Pd/C and hydrogen, and the like; with no co-solvent or in an appropriate solvent such as dichloromethane (DCM), methanol (MeOH), ethyl acetate (EtOAc), and the like

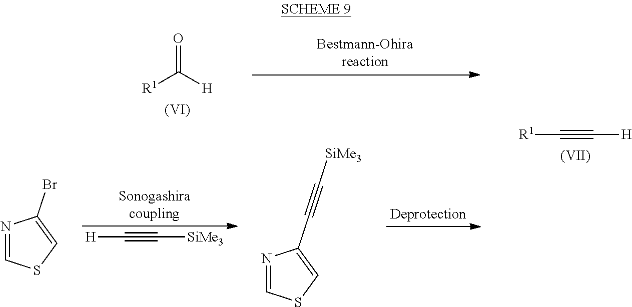

According to SCHEME 9, a compound of formula (VII) is prepared from a compound of formula (VI), where R¹ is a suitably substituted aryl, heteroaryl, alkyl, or cycloalkyaryl, heteroaryl, alkyl, or cycloalkyl as defined in Claim 1. For example, a commercially available or synthetically accessible compound of (VI), where R¹ is a suitably substituted aryl, heteroaryl, alkyl, or cycloalkyl; is subjected to Bestmann-Ohira conditions, using an alkynylating reagent such as dimethyl (1-diazo-2-oxopropyl)phosphonate and the like, in the presence of a suitable base such as potassium carbonate and the like, in a protic solvent such as methanol and the like. In an alternate method, a compound of formula (VII), where R¹ is thiazole, is prepared in two steps from 4-bromothiazole. In a first step, Sonogashira coupling between 4-bromothiazole and (trimethylsilyl)acetylene, using a palladium catalyst such as Pd(PPh₃)₂Cl₂ and the like, in the presence of a co-catalyst such as copper(I) iodide and the like, in a basic solvent such as triethylamine and the like, at a temperature of 85° C., using either conventional heating or a microwave reactor, provides 4-((trimethylsilyl)ethynyl)thiazole. Deprotection of the TMS protecting group is achieved employing methods known to one skilled in the art, for example, using a fluoride source such as TBAF and the like, in a solvent such as THF and the like, to provide a compound of formula (VII), where R¹ is thiazole

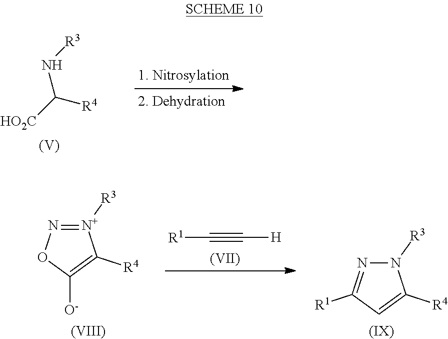

According to SCHEME 10, a pyrazole compound of formula (IX), where R¹ is a suitably substituted aryl, heteroaryl, alkyl, or cycloalkyl, and R³, and R⁴ are as defined in Claim 1, is prepared from commercially available or synthetically accessible appropriately substituted cyclic, bridged, or fused amino acid compound of formula (V). A compound of formula (VIII) is prepared from a compound of formula (V) via a nitroso source such as sodium nitrite and the like, in an acidic solvent such as aqueous hydrochloric acid and the like, followed by dehydration with a dehydrating reagent such as trifluoroacetic anhydride (TFAA) and the like, in a suitable solvent such as acetonitrile (ACN) and the like. A compound of formula (IX) is prepared by [3+2] cycloaddition between a compound of formula (VIII) and a compound of formula (VII), where R¹ is a suitably substituted aryl, heteroaryl, alkyl, or cycloalkyl; in a suitable solvent such as toluene, mesitylene, diphenyl ether, and the like; at a temperature between 150° C. and 210° C.; using either conventional heating or a microwave reactor, where R³ and R⁴ are as defined in Claim 1

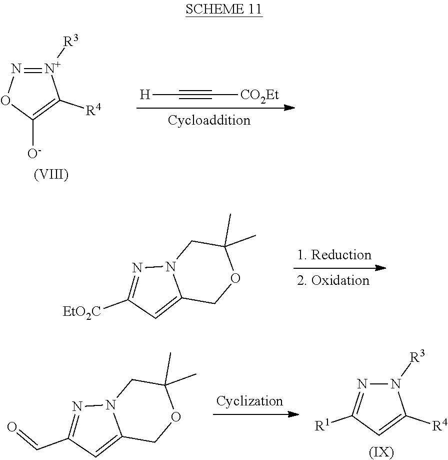

According to SCHEME 11, a compound of formula (VIII), where R³ and R⁴ come together to form

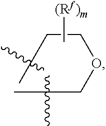

where R^{f} is CH₃, and m is 2, is reacted in a cycloaddition reaction with ethyl propiolate, employing methods previously described to provide ethyl 6,6-dimethyl-6,7-dihydro-4H-pyrazolo[5,1-c][1,4]oxazine-2-carboxylate. Ethyl 6,6-dimethyl-6,7-dihydro-4H-pyrazolo[5,1-c][1,4]oxazine-2-carboxylate is reduced using a reducing agent such as lithium borohydride and the like, with a protic additive such as methanol and the like, in a solvent such as THF and the like to provide (6,6-dimethyl-6,7-dihydro-4H-pyrazolo[5,1-c][1,4]oxazin-2-yl)methanol. (6,6-Dimethyl-6,7-dihydro-4H-pyrazolo[5,1-c][1,4]oxazin-2-yl)methanol is oxidized using an oxidizing agent such as manganese dioxide and the like, in a suitable solvent such as chloroform and the like, at a temperature of 60° C., to provide 6,6-dimethyl-6,7-dihydro-4H-pyrazolo[5,1-c][1,4]oxazine-2-carbaldehyde. 6,6-Dimethyl-6,7-dihydro-4H-pyrazolo[5,1-c][1,4]oxazine-2-carbaldehyde is reacted with 1-((isocyanomethyl)sulfonyl)-4-methylbenzene (TosMIC), in the presence of a base such as potassium carbonate, and the like; in a suitable solvent such as methanol, and the like; at a temperature of 80° C.; to provide a compound of formula (IX), where R¹ is oxazol-5-yl, R³ and R⁴ come together to form

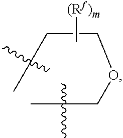

where R^{f} is CH₃, and m is 2

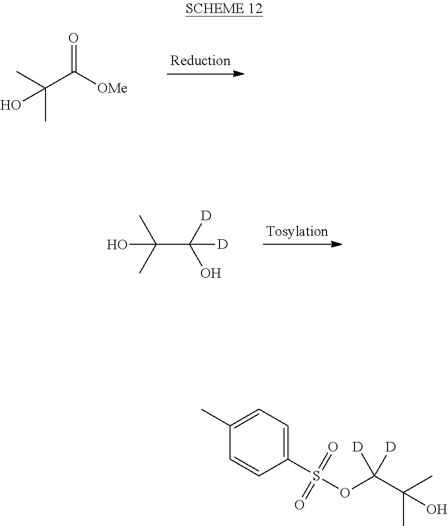

According to SCHEME 12, 2-methylpropane-1,1-d₂-1,2-diol is prepared from the reduction of methyl 2-hydroxy-2-methylpropanoate, using a reducing agent such a lithium aluminum deuteride and the like, in a solvent such a THF and the like, at a temperature ranging from 0° C. to room temperature. 2-Hydroxy-2-methylpropyl-1,1-d₂ 4-methylbenzenesulfonate is prepared from the treatment of 2-methylpropane-1,1-d₂-1,2-diol, using an electrophile such as toluenesulfonyl chloride (TsCl) and the like, an organic base such as triethylamine (NEt₃) and the like, 4-dimethyl aminopyridine (DMAP) and the like, in a solvent such as DCM and the like, at a temperature of RT ° C. for a period of 16 h

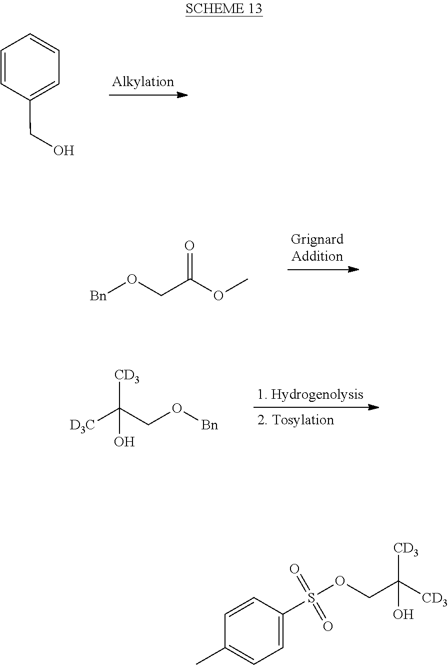 [SMILES: OCc1ccccc1 ; COC(=O)COCc1ccccc1 ; [2H]C([2H])([2H])C(O)(COCc1ccccc1)C([2H])([2H])[2H] ; [2H]C([2H])([2H])C(O)(COS(=O)(=O)c1ccc(C)cc1)C([2H])([2H])[2H]]

According to SCHEME 13, phenylmethanol is alkylated with an alkylating agent such methyl 2-bromoacetate, an inorganic base such as NaH, and the like, in a suitable solvent such as THF, and the like, at a temperature of about 0° C., for a period of 0.5 h to 18 h; to provide methyl 2-(benzyloxy)acetate. Methyl 2-(benzyloxy)acetate is reacted in a Grignard addition reaction with methyl-d₃-magnesium-iodide, in a suitable solvent such as THF, and the like, at a temperature ranging from 0° C. to rt ° C. for a period of 16 h, to provide 2-((benzyloxy)methyl)propan-1,1,1,3,3,3-d₆-2-ol. Deprotection of the benzyl protecting group is achieved employing a hydrogenation catalyst such as palladium on carbon (Pd/C) and the like, in a solvent such as EtOAc and the like, under an atmosphere (50 psi) of hydrogen (H₂), at a temperature of 50° C., for a period of 16 h, to provide 2-(methyl-d₃)propane-3,3,3-d₃-1,2-diol. 2-Hydroxy-2-(methyl-d₃)propyl-3,3,3-d₃ 4-methylbenzenesulfonate is prepared from 2-(methyl-d₃)propane-3,3,3-d₃-1,2-diol employing methods previously described.

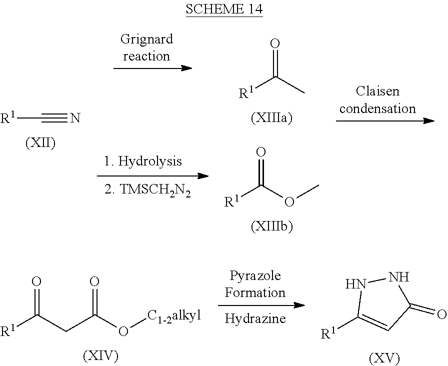

According to SCHEME 14, a compound of formula (XIIIa) is prepared from a commercially available or synthetically accessible compound of formula (XII), where R¹ is pyridyl substituted with one or two members each independently selected from halo, and OC₁₋₃alkyl; using a Grignard reagent such as methylmagnesium bromide, methylmagnesium iodide, and the like; in a suitable solvent such as THF and the like; at a temperature ranging from −70° C. to room temperature.

A compound of formula (XIIIb) is prepared from a commercially available or synthetically accessible compound of formula (XII), where R¹ is pyridyl substituted with one or two halo members in two steps. In a first step, hydrolysis of the nitrile, is achieved using an acidic catalyst such as sulfuric acid and the like, in aqueous solution, at a temperature of 110° C. to provide the acid. In a second step, a compound of formula (XIIIb) is prepared using a methylating reagent such as (trimethylsilyl)diazomethane and the like, in a solvent such as toluene and the like, with a protic co-solvent such as methanol and the like.

A compound of formula (XIV) is prepared from a Claisen condensation between a compound of formula (XIIIa) or (XIIIb); dimethyl carbonate or ethyl acetate; a suitable base such as potassium tert-pentoxide, sodium hydride, and the like, at temperatures ranging from −10° C. to 50° C. A pyrazolone compound of formula (XV) is prepared from a compound of formula (XIV), using a commercially available hydrazine source such as hydrazine hydrate and the like, in a solvent such as acetic acid and the like, at a temperature of 80° C.

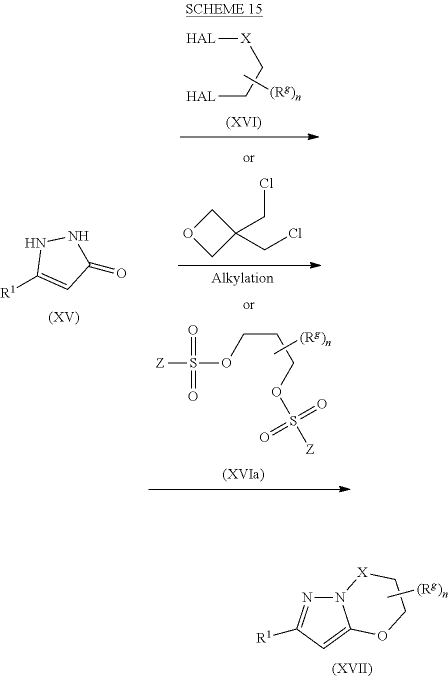

According to SCHEME 15, a compound of formula (XVII) where R¹ is a suitably substituted phenyl or pyridyl as defined in Claim 1, n is 1, or 2, R^{g} is H or C₁₋₃alkyl, and X is CH₂, CH(CH₃), or CH₂CH₂, is prepared by alkylating a compound of formula (XV) with a compound of formula (XVI) (or 3,3-bis(chloromethyl)oxetane), employing methods known to one skilled in the art. For example, a compound of formula (XV) where R¹ is a suitably substituted phenyl or pyridyl as defined in Claim 1, is reacted with an alkyl electrophile compound of formula (XVI), where HAL is independently Cl and Br, n is 1, or 2, R^{g} is H or C₁₋₃alkyl, and X is CH₂, CH(CH₃), or CH₂CH₂, with a suitable base such as K₂CO₃, Cs₂CO₃, sodium hydride (NaH), and the like; with or without an additive such as KI; a suitable solvent such as ACN, THF, DMF, DCM, N-methyl pyrrolidone (NMP), and the like; at temperatures ranging from room temperature to 180° C.; for a period of 4-16 h; employing conventional heating or a microwave heating, to provide a compound of formula (XVII). In a similar fashion, 3,3-bis(chloromethyl)oxetane is reacted with 5-(4-fluorophenyl)-1,2-dihydro-3H-pyrazol-3-one to provide 2′-(4-fluorophenyl)-5′H,7′H-spiro[oxetane-3,6′-pyrazolo[5,1-b][1,3]oxazine].

Similarly, cis-1,2-bis(bromomethyl)cyclopropane (commercially available or synthetically accessible using methods known to one skilled in the art from 3-oxabicyclo[3.1.0]hexane-2,4-dione) is reacted with 5-(5-fluoropyridin-2-yl)-1,2-dihydro-3H-pyrazol-3-one employing methods as previously described to provide 2-(5-fluoropyridin-2-yl)-5a,6,6a,7-tetrahydro-5H-cyclopropa[e]pyrazolo[5,1-b][1,3]oxazepane.

A compound of formula (XVII) wherein R¹ is a suitably substituted phenyl or pyridyl as defined in Claim 1, n is 1, or 2, and R^{g} is as defined in Claim 1; is prepared by reaction of a compound of formula (XVIa) (prepared by reaction of a suitably substituted synthetically accessible or commercially available propane-diol with TosCl or MsCl in the presence of a base such as triethylamine and the like, in a suitable solvent such as DCM and the like) where Z is methyl or p-tolyl and R^{g} is as defined in claim, with a compound of formula (XV) in the presence of a base such as Cs₂CO₃ and the like; in a solvent such as DMF and the like; at a temperature ranging from 50-70° C.

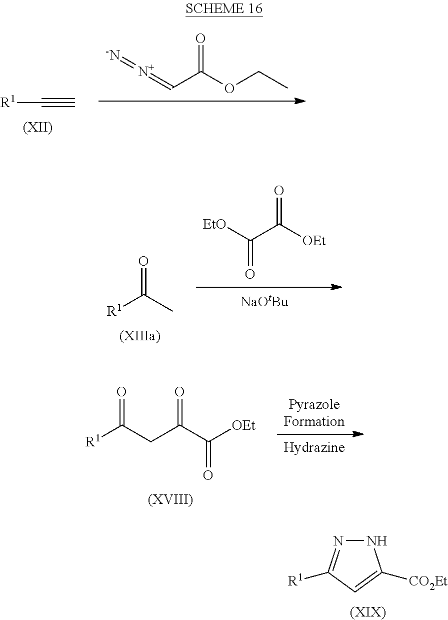

According to SCHEME 16, a compound of formula (VII), where R¹ is a suitably substituted phenyl or pyridyl as defined in Claim 1, is reacted with ethyl 2-diazoacetate, in a high-boiling solvent such as toluene, at a temperature of 105° C., employing microwave or conventional heating, to afford a pyrazole compound of formula (XIX). Alternately, a pyrazole compound of formula (XIX) is prepared in two steps from a commercially available or synthetically accessible compound of formula (XIIIa), where R¹ is a suitably substituted phenyl or pyridyl as defined in claim. In a first step, condensation of a compound of formula (XIIIa) with diethyl oxalate, a suitable base such as sodium tert-butoxide (NaOtBu) and the like, in a suitable solvent such as ethanol (EtOH), at room temperature, affords a compound of formula (XVIII). A pyrazole compound of formula (XIX) is prepared by reaction of a compound of formula (XVIII) with hydrazine, in a solvent such as acetic acid, and the like, at room temperature

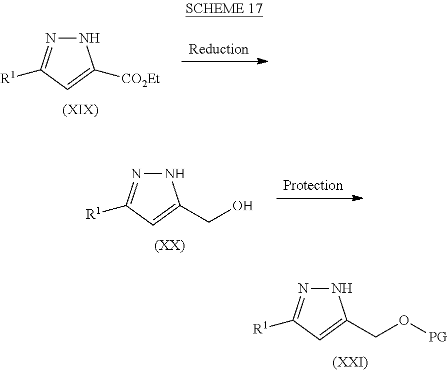

According to SCHEME 17, a compound of formula (XIX), where R¹ is a suitably substituted phenyl or pyridyl as defined in Claim 1, is reduced to a compound of formula (XX) employing a suitable reductant known to one skilled in the art such as LiAlH₄, lithium aluminum deuteride, DIBAL-H, BH₃·THF, NaBH₄, and the like; in a suitable solvent such as THF, toluene, MeOH, and the like, at temperatures ranging from 0° C. to rt. A compound of formula (XX) is protected with a silyl protecting group such as t-butyldiphenylsilyl ether (TBDPS), trimethylsilyl (TMS), tert-butyldimethylsilyl (TBDMS), and triisopropylsilyl (TIPS) ethers, preferably TBDPS. For example reaction of a compound of formula (XX) with tert-butyldimethylsilyl chloride, (TBDMSCl); a suitable base such as imidazole, triethylamine, DMAP, and the like; in a suitable solvent such as DCM, dimethylformamide (DMF), tetrahydrofuran (THF), and the like; for a period of about 2 h, affords a compound of formula (XXI), where PG is TBDMS

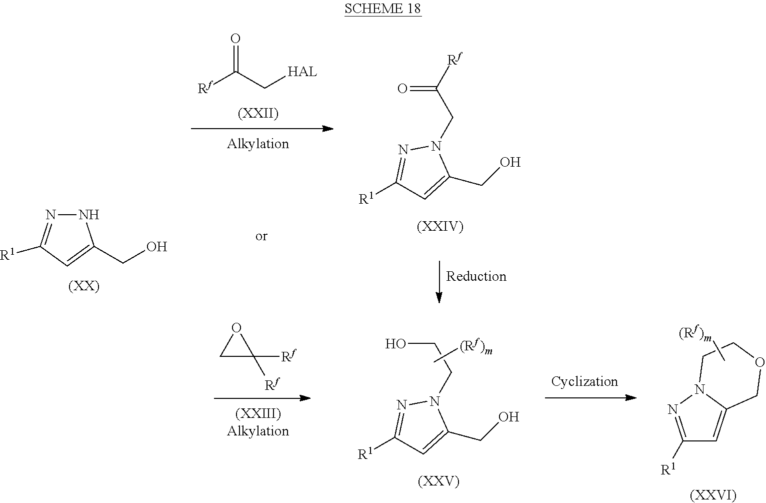

According to SCHEME 18, a compound of formula (XX), where R¹ is a suitably substituted phenyl or pyridyl as defined in Claim 1, is alkylated with an alpha halo ketone compound of formula (XXII), where HAL is Br, and R^{f} is C₁₋₃alkyl or C₃₋₆cycloalkyl; with a suitable base such as K₂CO₃, Cs₂CO₃, and the like; a suitable solvent such as ACN, THF, DMF, DCM, and the like; at rt; for a period of 4-16 h; to provide a compound of formula (XXIV). Reduction of a compound of formula (XXIV) is achieved employing a suitable reductant known to one skilled in the art such as LiAlH₄, DIBAL-H, BH₃·THF, or NaBH₄, in a suitable solvent such as THF, toluene, or MeOH, and the like; to provide a compound of formula (XXV).

A compound of formula (XX), where R¹ is a suitably substituted phenyl or pyridyl as defined in Claim 1, is alkylated with an oxirane compound of formula (XXIII), where each R^{f} is independently H and C₁₋₃alkyl; employing alkylation conditions previously described to provide a compound of formula (XXV), where each R^{f} is independently H or C₁₋₃alkyl. Cyclization of a compound of formula (XXV) under acidic or basic conditions employing H₂SO₄, H₃PO₄, ZnCl₂, a combination of KOH and TsCl, a combination of KOtBu and MsCl, and the like; optionally in a solvent such as toluene, 1,2-dichloroethane, 1,4-dioxane, and the like; at temperatures from 90° C. to 120° C., for a period of 6 to 18 h; provides a compound of formula (XXVI), where each R^{f} is independently H, C₁₋₃alkyl, C₃₋₆cycloalkyl, and m is 1, 2, or 3.

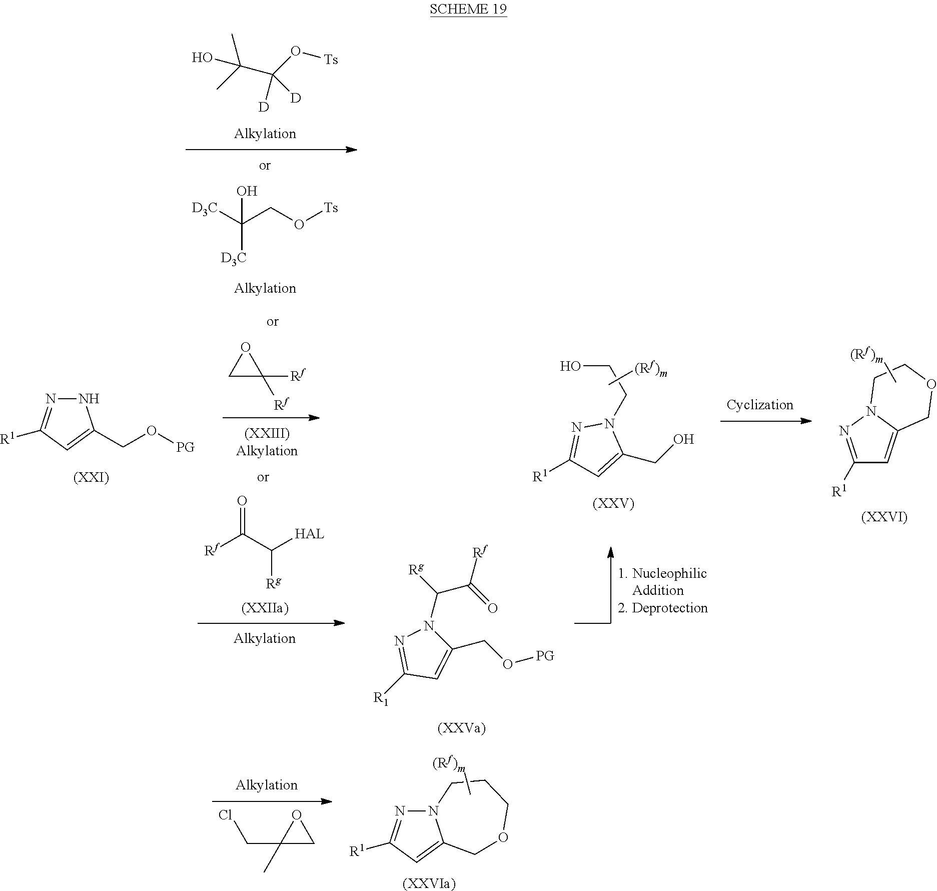

According to SCHEME 19, a compound of formula (XXI), where R¹ is a suitably substituted phenyl or pyridyl as defined in Claim 1, and PG is TBDMS, is reacted with 2-hydroxy-2-methylpropyl-1,1-d₂ 4-methylbenzenesulfonate or 2-hydroxy-2-(methyl-d₃)propyl-3,3,3-d₃ 4-methylbenzenesulfonate, employing alkylation conditions known to one skilled in the art. For example, alkylation of a compound of formula (XXI), where R¹ is a suitably substituted phenyl or pyridyl as defined in Claim 1, and PG is TBDMS, with 2-hydroxy-2-methylpropyl-1,1-d₂ 4-methylbenzenesulfonate or 2-hydroxy-2-(methyl-d₃)propyl-3,3,3-d₃ 4-methylbenzenesulfonate; utilizing a suitable inorganic base such as Cs₂CO₃, and the like; potassium iodide (KI) and the like; in a suitable solvent such as dimethylacetamide (DMA), and the like; at a temperature of 120° C., under microwave irradiation for a period of 0.5 h; affords a compound of formula (XXV), where each R^{f} is independently H, D, CH₃, and CD₃, and m is 1, 2, 3 or 4.

A compound of formula (XXI), where R¹ is a suitably substituted phenyl or pyridyl as defined in Claim 1, and PG is TBDMS, is reacted with an oxirane compound of formula (XXIII), where each R^{f} is independently H, C₁₋₃alkyl, and C₁₋₃haloalkyl; employing alkylation conditions previously described to provide a compound of formula (XXV), where each R^{f} is independently H, C₁₋₃alkyl, and C₁₋₃haloalkyl.

A compound of formula (XXI), where R¹ is a suitably substituted phenyl or pyridyl as defined in Claim 1, is alkylated with an alpha halo ketone compound of formula (XXIIa), where HAL is Br, R^{f} is C₁₋₃alkyl, and R^{g} is H or CH₃; employing conditions known to one skilled in the art or previously described, to provide a compound of formula (XXVa). A compound of formula (XXV), where m is 1, 2, 3 or 4, and each R^{f} is independently H or C₁₋₃alkyl, is prepared in two steps from a compound of formula (XXVa). In a first step, a compound of formula (XXVa) is reacted in nucleophilic addition reaction with trimethyl(trifluoromethyl)silane, or an alkyllithium or Grignard reagent, such as MeLi, MeMgBr, and the like; in a suitable solvent such as THF, DCM, and the like; at −70° C. to room temperature; for a period of 3 h to 16 h. Subsequent deprotection of the silyl protecting group is achieved using tetra-n-butylammonium fluoride (TBAF), in a suitable solvent such as THF and the like; at room temperature for a period of 1 h, affords a compound of formula (XXV). A compound of formula (XXV) is cyclized to a compound of formula (XXVI) employing methods previously described.

A compound of formula (XXI), where R¹ is a suitably substituted phenyl or pyridyl as defined in Claim 1, is alkylated, and cyclized with 2-(chloromethyl)-2-methyloxirane, employing conditions previously described. For example, reaction of a compound of formula (XXI), where R¹ is a suitably substituted phenyl or pyridyl as defined in Claim 1; with 2-(chloromethyl)-2-methyloxirane; a suitable base such as Cs₂CO₃, and the like; in a suitable solvent such as DMF, and the like; at temperatures ranging from 50° C. for 2 hrs employing conventional heating; followed by microwave irradiation at 120° C. for 10 minutes; provides a compound of formula (XXVIa), where m is 2, and R^{f} is independently C₁₋₃alkyl and OH

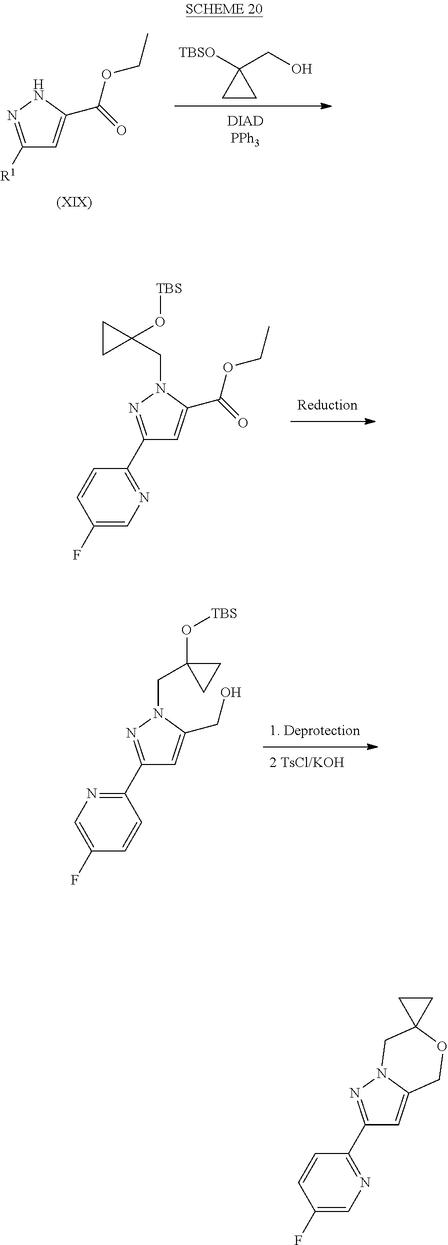

According to SCHEME 20, (1-((tert-butyldimethylsilyl)oxy)cyclopropyl)methanol (commercially available or prepared by ester reduction of methyl 1-((tert-butyldimethylsilyl)oxy)cyclopropanecarboxylate) is reacted in a Mitsunobu reaction with a compound of formula (XIX), where R¹ is 5-fluoropyridin-2-yl; using a coupling reagent such as DIAD and the like, an additive such as PPh₃ and the like, in a suitable solvent such as THF and the like; to afford ethyl 1-((1-((tert-butyldimethylsilyl)oxy)cyclopropyl)methyl)-3-(5-fluoropyridin-2-yl)-1H-pyrazole-5-carboxylate. Reduction of the ester of ethyl 1-((1-((tert-butyldimethylsilyl)oxy)cyclopropyl)methyl)-3-(5-fluoropyridin-2-yl)-1H-pyrazole-5-carboxylate is achieved employing methods known to one skilled in the art such as LiAlH₄; in a suitable solvent such as THF, and the like; at temperatures ranging from 0° C. to rt; to afford (1-((1-((tert-Butyldimethylsilyl)oxy)cyclopropyl)methyl)-3-(5-fluoropyridin-2-yl)-1H-pyrazol-5-yl)methanol. Deprotection of the silyl protecting group is achieved employing TBAF/THF to afford 1-((3-(5-fluoropyridin-2-yl)-5-(hydroxymethyl)-1H-pyrazol-1-yl)methyl)cyclopropanol. Dehydrative cyclization of 1-((3-(5-fluoropyridin-2-yl)-5-(hydroxymethyl)-1H-pyrazol-1-yl)methyl)cyclopropanol, using a reagent such as TsCl, in the presence of a base such as KOH, in a suitable solvent such as dioxane, at a temperature of 100° C., affords 2′-(5-fluoropyridin-2-yl)-4′,7′-dihydrospiro[cyclopropane-1,6′-pyrazolo[5,1-c][1,4]oxazine]

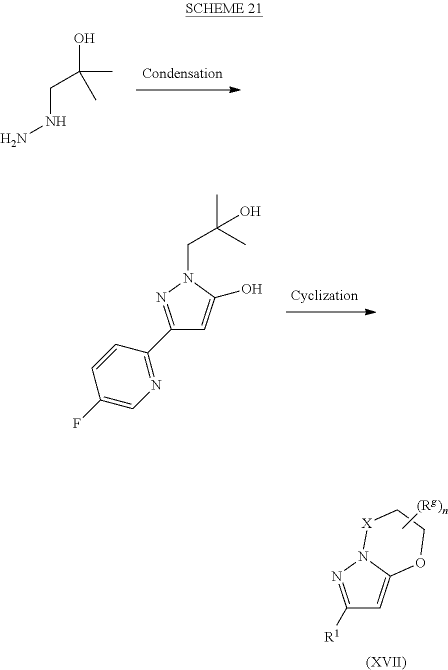

According to SCHEME 21, 1-hydrazineyl-2-methylpropan-2-ol (commercially available or prepared via the reaction between 2,2-dimethyloxirane and hydrazine hydrate employing methods known to one skilled in the art) is condensed with methyl 3-(5-fluoropyridin-2-yl)-3-oxopropanoate, employing methods previously described to provide 3-(5-fluoropyridin-2-yl)-1-(2-hydroxy-2-methylpropyl)-1H-pyrazol-5-ol. A compound of formula (XVII), where X is a bond, R^{g} is CH₃, and n is 2; is prepared by dehydrative cyclization of 3-(5-fluoropyridin-2-yl)-1-(2-hydroxy-2-methylpropyl)-1H-pyrazol-5-ol, using a dehydrating reagent such as polyphosphoric acid, neat, at a temperature of 120° C.

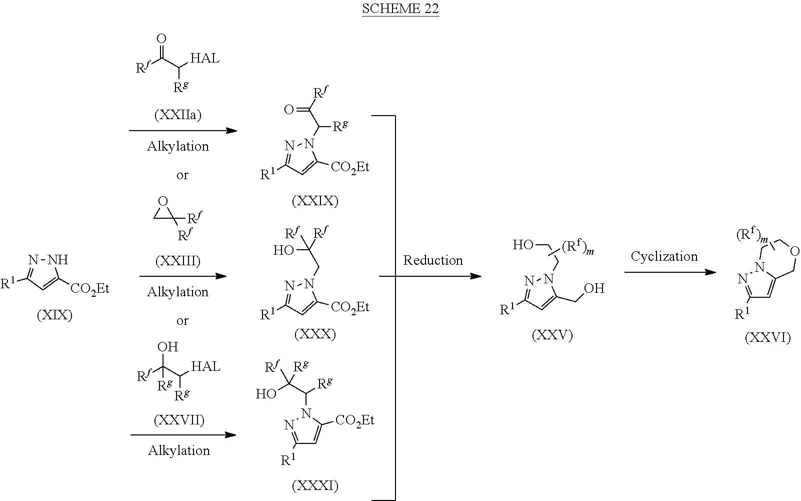

According to SCHEME 22, a compound of formula (XIX), where R¹ is a suitably substituted phenyl or pyridyl as defined in Claim 1, is alkylated with an alpha halo ketone compound of formula (XXIIa), where HAL is Br, R^{f} is C₁₋₃alkyl, and R^{g} is H or CH₃; employing conditions known to one skilled in the art or previously described, to provide a compound of formula (XXIX).

A compound of formula (XIX), where R¹ is a suitably substituted phenyl or pyridyl as defined in Claim 1, is alkylated with an oxirane compound of formula (XXIII), where each R^{f} is independently H, C₁₋₃alkyl and C₁₋₃haloalkyl; employing alkylation conditions previously described to provide a compound of formula (XXX).

A compound of formula (XIX), where R¹ is a suitably substituted phenyl or pyridyl as defined in Claim 1, is alkylated with 2-halo alcohol compound of formula (XXVII), where HAL is Br or Cl, each R^{g} is independently H or CH₃, and R^{f} is C₁₋₃haloalkyl; employing conditions previously described, to provide a compound of formula (XXXI). It is possible that under the alkylation conditions that the ethyl ester is hydrolyzed to the acid (CO₂H).

Reduction of compounds of formula (XXIX), (XXX), and (XXI), employing methods known to one skilled in the art, or as previously described affords a compound of formula (XXV). Subsequent cyclization of a compound of formula (XXV), employing conditions previously described affords a compound of formula (XXVI), where m is 1, 2, or 3, and each R^{f} is independently H, C₁₋₃alkyl, C₁₋₃haloalkyl, and two R^{f} members come together to form a cyclopropyl

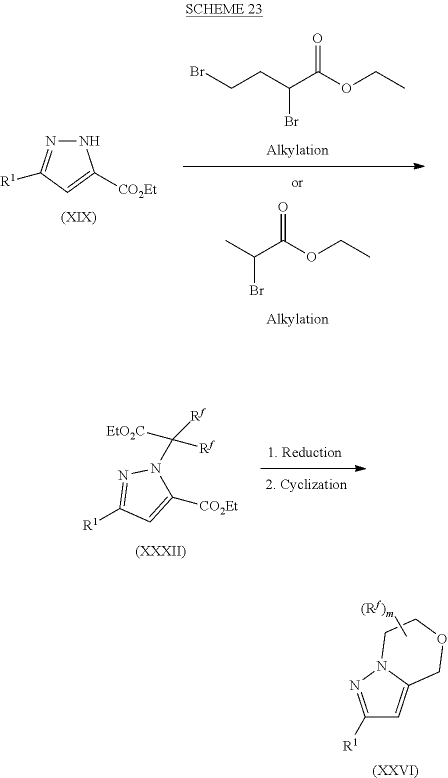

According to SCHEME 23, a compound of formula (XIX), where R¹ is a suitably substituted phenyl or pyridyl as defined in Claim 1, is alkylated with ethyl 2,4-dibromobutanoate, employing conditions previously described to provide a compound of formula (XXXII), where two R^{f} members come together to form a cyclopropyl ring. Similarly, a compound of formula (XIX) is alkylated with ethyl 2-bromopropanoate, employing conditions previously described to provide a compound of formula (XXXII), where one R^{f} member is H and the other R^{f} member is C₁₋₃alkyl. A compound of formula (XXXII), where one R^{f} member is H and the other R^{f} member is C₁₋₃alkyl is further alkylated with Mel utilizing a base such as LiHMDS, LDA, and the like; in a solvent such as THF, Et₂O, or toluene, and the like; at temperatures ranging from −78° C. to rt, to provide a compound of formula (XXXII), where each R^{f} is C₁₋₃alkyl. Subsequent reduction of the ester followed by cyclization, employing conditions previously described, provides a compound of formula (XXVI)

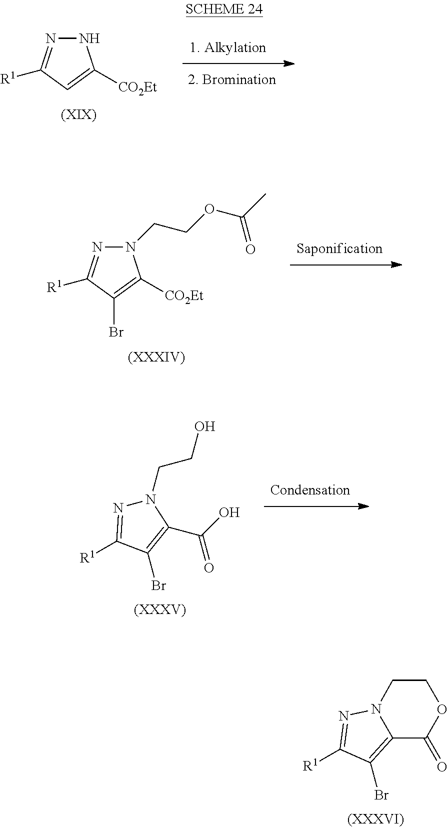

According to SCHEME 24, a compound of formula (XIX) is alkylated using an alkylating reagent such as (2-bromoethoxy)(tert-butyl)dimethylsilane and the like, in the presence of a base such as Cs₂CO₃ and the like, in a suitable solvent such as N,N-dimethylacetamide and the like. Subsequent bromination and acetylation is achieved in one step, employing a brominating reagent such as N-bromosuccinimide and the like, with acetic acid as solvent; at a temperature of 150° C., employing microwave irradiation; for a period of 0.1 h; to provide a compound of formula (XXXIV). A compound of formula (XXXIV) is saponified using a suitable base such as lithium hydroxide monohydrate, aqueous sodium hydroxide (NaOH), and the like; in a suitable solvent such as EtOH, water, or a mixture thereof, at a temperature of 60° C. to 80° C.; for a period of 1-6 h; to provide a compound of formula (XXXV). Intramolecular condensation of a compound of formula (XXXV) using a dehydrating agent such as T3P® and the like, in the presence of a base such as triethylamine and the like, in a solvent such as DMF and the like, affords a compound of formula (XXXVI)

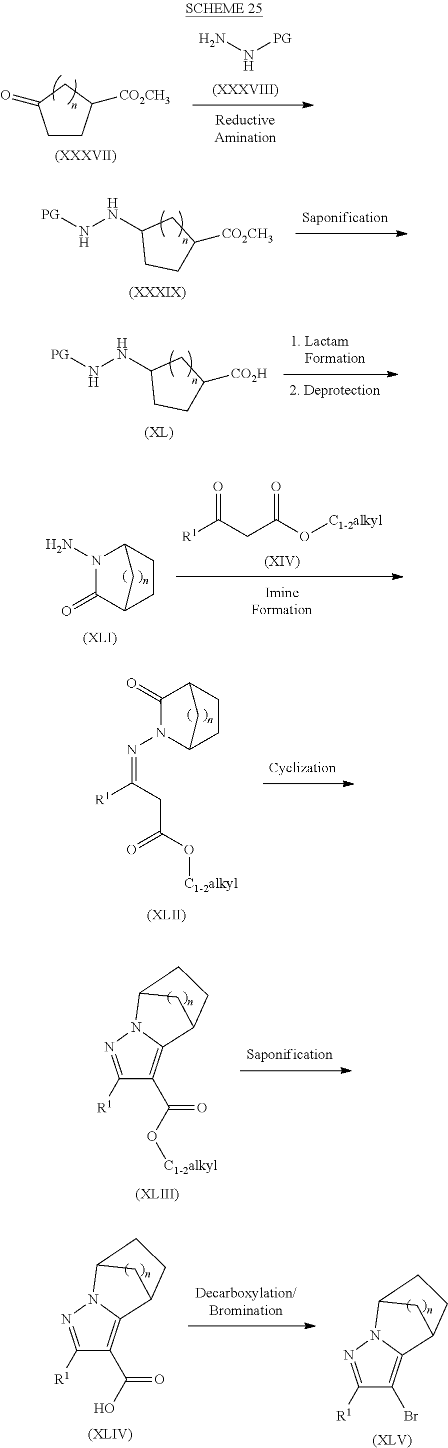

According to SCHEME 25, a commercially available or synthetically accessible compound of formula (XXXVII), where n is 1 or 2, is reacted with a hydrazine compound of formula (XXXVIII), where PG is CBz, employing reductive amination conditions known to one skilled in the art. For example, a compound of formula (XXXVII), where n is 1 or 2, is reacted with N-benzyloxycarbonylhydrazine; a reducing agent such as sodium cyanoborohydride (NaBH₃CN), and the like; in the presence of an acidic additive such as acetic acid, and the like; in a suitable solvent such as methanol, and the like; at room-temperature; for a period of 14-24 h; to provide a compound of formula (XXXIX), where PG is CBz. A compound of formula (XXXIX) is saponified, employing conditions known to one skilled in the art, or as previously described to provide a compound of formula (XL). A lactam of formula (XLI) is prepared in two steps from a compound of formula (XL). In a first step, a compound of formula (XL) is reacted with a peptide coupling reagent such as T3P®, and the like; in the presence of a suitable base such as triethylamine, and the like; in a suitable aprotic solvent such as DCM and the like. In a second step, deprotection of the CBz protecting group is achieved using a catalyst such as Pd/C and the like; in the presence of an acidic additive such asp-toluenesulfonic acid (TsOH), and the like; in a suitable solvent such as methanol, and the like; under an atmosphere of hydrogen; to provide a lactam compound of formula (XLI).

Imine condensation of a compound of formula (XLI), and a commercially available or synthetically accessible compound of formula (XIV), where R¹ is a suitably substituted phenyl or pyridyl as defined in Claim 1; in the presence of a dehydrating agent such as molecular sieves, and the like; in a suitable solvent such as pyridine, and the like; affords a compound of formula (XLII). A compound of formula (XLII) is reacted in a cyclic condensation reaction employing conditions known to one skilled in the art or as previously described, to afford a compound of formula (XLIII). A compound of formula (XLIII) is saponified, employing conditions previously described, to provide a compound of formula (XLIV).

A compound of formula (XLIV) is reacted in a decarboxylative bromination reaction using an electrophilic brominating reagent such as N-bromosuccinimide, and the like; in a suitable solvent such as DMF, and the like; at a temperature of 50° C.; to provide a compound of formula (XLV)

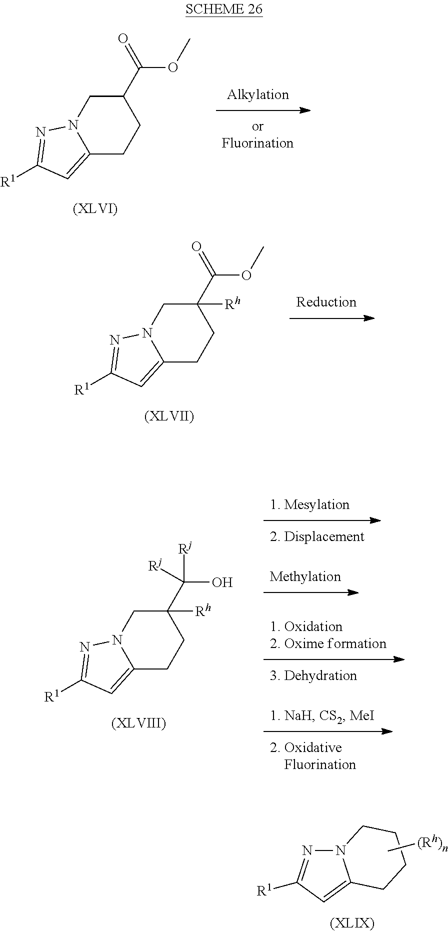

According to SCHEME 26, a compound of formula (XLVI) is alkylated with a suitable alkylating agent such as iodomethane, CD₃I, and the like; a suitable base such as lithium bis(trimethylsilyl)amide, NaH, and the like; in a suitable solvent such as THF, and the like; at temperatures ranging from −70° C. to ambient temperature; for a period of 3 to 6 h; to provide a compound of formula (XLVII), where R^{h} is C₁₋₃alkyl or CD₃. A compound of formula (XLVI) is fluorinated with a suitable fluorinating agent such as NFSI, and the like; a suitable base such as lithium bis(trimethylsilyl)amide (LDA), and the like; in a suitable solvent such as THF, and the like; a temperatures ranging from −70° C. to room temperature to afford a compound of formula (XLVII), were R^{h} is F. Reduction of the ester to the alcohol is achieved employing reduction conditions known to one skilled in the art. For example, a compound of formula (XLVII), where R^{h} is F, C₁₋₃alkyl, or CD₃; is reduced with a suitable reducing agent such as LiAlD₄, LiBH₄, and the like; in a suitable solvent such as THF, and the like; at temperatures ranging from 0° C. to room temperature; to afford a compound of formula (XLVIII), R^{h} is F, C₁₋₃alkyl, or CD₃, and each R^{j} is H or D.

A compound of formula (XLIX), where R^{h} is C₁₋₃alkyl, and n is 2; is prepared in two steps from a compound of formula (XLVIII), where R^{h} is C₁₋₃alkyl, and each R^{j} is H or D. In a first step, sulfonylation of a compound of formula (XLVIII) is achieved with methanesulfonyl chloride; in a suitable solvent such as dichloromethane, and the like; a tertiary amine base such as triethylamine, and the like; at temperatures ranging from 0° C. to ambient room temperature. Subsequent displacement of the methanesulfonate of a compound of formula (XLVIII), is achieved employing NaI; Zn (dust); in a suitable polar aprotic solvent such as HMPA, and the like; at temperatures ranging from room temperature to 125° C.; for a period of 72 h; to afford a compound of formula (XLIX), where R^{h} is C₁₋₃alkyl and n is 2. Alternately, the methanesulfonate of a compound of formula (XLVIII) where each R¹ is H or D, is reacted with tetrabutylammonium fluoride trihydrate; in a suitable solvent such as methyl ethyl ketone; at a temperature ranging from room temperature to 90° C.; for a period of 24; to provide a compound of formula compound of formula (XLIX), where each R^{h} is independently C₁₋₃alkyl and C₁₋₃haloalkyl, and n is 2, wherein the C₁₋₃alkyl and C₁₋₃haloalkyl is optionally substituted with one or more deuterium atoms.

A compound of formula (XLVIII), R^{h} is F or C₁₋₃alkyl, is alkylated with a suitable alkylating agent such as iodomethane; a suitable base such as NaH; in a suitable solvent such as THF, and the like; at temperatures ranging from 0° C. to room temperature; to afford a compound of formula (XLIX), where n is 2, and one R^{f} member is CH₂OCH₃, and one R^{f} member is either F or C₁₋₃alkyl.

A compound of formula (XLVIII), R^{h} is F, is reacted with 2,2-difluoro-2-(fluorosulfonyl)acetic acid; in the presence of a suitable catalyst such as CuI, and the like; in a suitable solvent such as MeCN, and the like; to provide a compound of formula (XLIX), where n is 2, and one R^{f} member is CH₂OCHF₂, and one R^{f} member is F.

A compound of formula (XLIX), where n is 2, one R^{h} member is F, and the other R^{h} member is CH₂OCF₃, is prepared in two steps from a compound of formula (XLVIII), where R^{h} is F. In a first step, deprotonation of a compound of formula (XLVIII) with a suitable base such as NaH, and the like; followed by treatment with carbon disulfide; then treatment with Mel; in a suitable solvent such as THF, and the like; affords a S-methyl carbonodithioate intermediate compound. In a second step, the S-methyl carbonodithioate intermediate is reacted under oxidative fluorination conditions known to one skilled in the art. For example, using a fluorine source such as HF-pyridine, and the like; an oxidant such as 1,3-dibromo-5,5-dimethylimidazolidine-2,4-dione, and the like; in a suitable solvent such as DCM, and the like; to afford a compound of formula (XLIX), where n is 2, one R^{h} member is F, and the other R^{h} member is CH₂OCF₃.

A compound of formula (XLIX), where n is 2, R^{h} is independently CN and CD₃, is prepared in three steps from a compound of formula (XLVIII), where R^{h} is CD₃. In a first step, oxidation of a compound of formula (XLVIII), using an oxidizing agent such Dess-Martin periodinane (DMP); in a suitable solvent such as dichloromethane, and the like; at temperatures ranging from about 0° C. to about 25° C.; for a period of approximately 0.5 to 4 hours; to provide the corresponding aldehyde intermediate. In a second step, oxime formation is achieved employing hydroxylamine hydrochloride; in the presence of a weak base such as sodium acetate and the like; in a suitable solvent such as THF, and the like; at a temperature of about 50° C.; for a period of about 4-7 hours. In a third step, dehydration of the oxime is achieved employing a dehydrating agent such as SOCl₂ and the like; in the presence of a base such as triethylamine, and the like; in a suitable solvent such as THF, and the like; to provide a compound of formula (XLIX), where n is 2, R^{h} is independently CN and CD₃

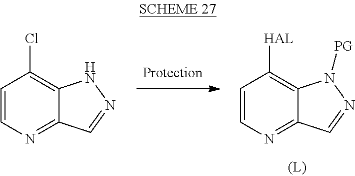

According to SCHEME 27, 7-chloro-1H-pyrazolo[4,3-b]pyridine is reacted with a suitable alkylating agent, such as (2-(chloromethoxy)ethyl)trimethylsilane, employing a base such as NaH, in a suitable solvent such as THF, at temperatures ranging from 0° C. to rt, for 1 to 12 hours, to afford a compound of formula (L), where PG is SEM and HAL is Cl

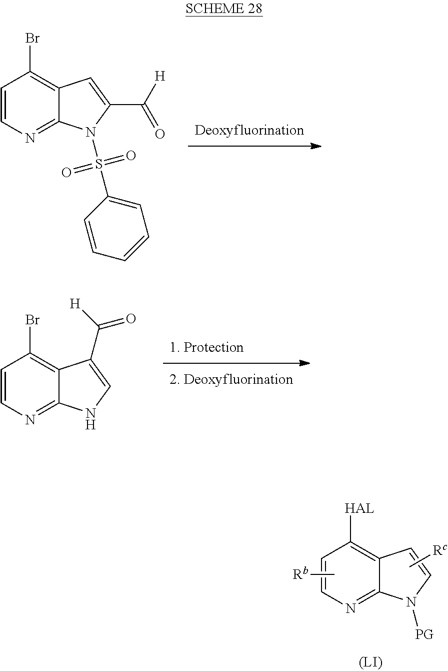

According to SCHEME 28, commercially available or synthetically accessible 4-bromo-1-(phenylsulfonyl)-1H-pyrrolo[2,3-b]pyridine-2-carbaldehyde is deoxyfluorinated by reaction with a reagent such as XtalFluor-E®, in the presence of a promoter such as triethylamine trihydrofluoride, in a suitable solvent such as CH₂Cl₂, at temperatures ranging from 0° C. to rt; to afford a compound of formula (LI), where R^{b} is H, HAL is Br, R^{c} is CHF₂, and PG is benzenesulfonyl (Bs).

In a similar fashion, a compound of formula (LI), where R^{b} is H, HAL is Br, R^{c} is CHF₂, and PG is SEM is prepared in two steps. In a first step, 4-bromo-1H-pyrrolo[2,3-b]pyridine-3-carbaldehyde is protected with a suitable nitrogen protecting group (PG) such as SEM (2-(trimethylsilyl)ethoxymethyl), employing methods known to one skilled in the art. For example, 4-bromo-1H-pyrrolo[2,3-b]pyridine-3-carbaldehyde is reacted with 2-chloromethoxyethyl)trimethylsilane, in the presence of a base such as NaH, and the like, in a suitable solvent such as DMF, and the like; at temperatures ranging from 0° C. to rt. In a second step, 4-bromo-1-((2-(trimethylsilyl)ethoxy)methyl)-1H-pyrrolo[2,3-b]pyridine-3-carbaldehyde is fluorinated with a fluorinating agent such as, DAST, XtalFluor®, Deoxo-Fluor®, and the like, in a suitable solvent such as DCM, and the like, at temperatures ranging from −78° C. to 50° C., for a period of 2-24 h. In a preferred method, 4-bromo-1-((2-(trimethylsilyl)ethoxy)methyl)-1H-pyrrolo[2,3-b]pyridine-3-carbaldehyde, is reacted with a fluorinating agent such as DAST, in a solvent suitable solvent such as DCM, at room temperature, for 20 h, to provide a compound of formula (LI), where R^{b} is H, R^{c} is CF₂H, and PG is SEM.

Additionally, 4-bromo-1H-pyrazolo[3,4-b]pyridine is treated with a fluorinating agent such as XeF₂ and the like; in a suitable solvent such as CCl₄ and the like; at a temperature of 40° C.; to provide 4-bromo-3-fluoro-1H-pyrazolo[3,4-b]pyridine

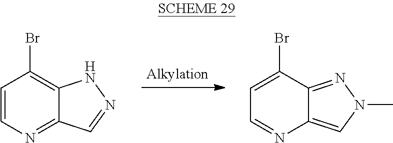

According to SCHEME 29, 7-bromo-1H-pyrazolo[4,3-b]pyridine is alkylated with a suitable alkylating agent such as iodomethane, employing conditions previously described to afford 7-bromo-2-methyl-2H-pyrazolo[4,3-b]pyridine

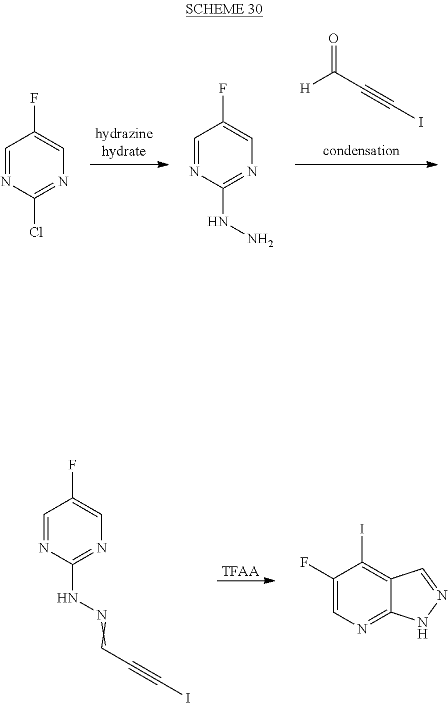 [SMILES: Fc1cnc(Cl)nc1 ; NNc1ncc(F)cn1 ; O=CC#CI ; Fc1cnc(NN=CC#CI)nc1 ; "TFAA" ; Fc1cnc2[nH]ncc2c1I]

According to SCHEME 30, 2-chloro-5-fluoropyrimidine is reacted in an S_{N}Ar reaction with hydrazine hydrate, in a solvent such as EtOH and the like, at a temperature of 60° C., to provide 5-fluoro-2-hydrazineylpyrimidine. Condensation reaction between 5-fluoro-2-hydrazineylpyrimidine and 3-iodopropiolaldehyde (prepared according to methods described in the art in two steps from trimethylsilylacetylene) in the presence of an additive such as TFA and the like, in a suitable solvent such as THF and the like, provides 5-fluoro-2-(2-(3-iodoprop-2-yn-1-ylidene)hydrazineyl)pyrimidine. 5-Fluoro-2-(2-(3-iodoprop-2-yn-1-ylidene)hydrazineyl)pyrimidine is reacted in the presence of a dehydrating agent such as TFAA and the like, in the presence of an additive such as 3-pentanone and the like, in a suitable solvent such as THF and the like, at a temperature of 60° C., to provide 5-fluoro-4-iodo-1H-pyrazolo[3,4-b]pyridine.

In a similar fashion, 5-fluoro-4-iodo-6-methyl-1H-pyrazolo[3,4-b]pyridine is prepared according to methods described above starting from 2-chloro-5-fluoro-4-methylpyrimidine (prepared by reacting 2,4-dichloro-5-fluoropyrimidine with methylmagnesium chloride, in the presence of a catalyst such as Fe(acac)₃ employing methods known to one skilled in the art)

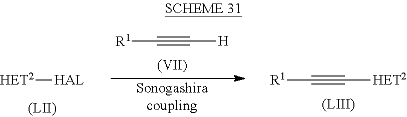

According to SCHEME 31, a commercially available or synthetically accessible compound of formula (LII), where HET² is a suitably substituted pyridyl as defined in Claim 1, 1-methyl-1H-pyrazolo[4,3-b]pyridine or 1-((2-(trimethylsilyl)ethoxy)methyl)-1H-pyrrolo[2,3-b]pyridine-3-carbonitrile; is coupled with a compound of formula (VII), where R¹ is a suitably substituted pyridyl as described in claim 1; under Sonogashira conditions, employing a palladium catalyst XPhos Pd G3, PdCl₂(Cy*Phine)₂, and the like; a suitable base such as Cs₂CO₃, Et₃N, and the like; with or without the presence of a copper additive such as CuI; in a suitable solvent such as MeCN, DMF, and the like; at a temperature of 80° C. to 125° C.; for a period of 2 to 8 h; to afford a compound of formula (LIII), where HET² is optionally substituted with an appropriate nitrogen protecting group

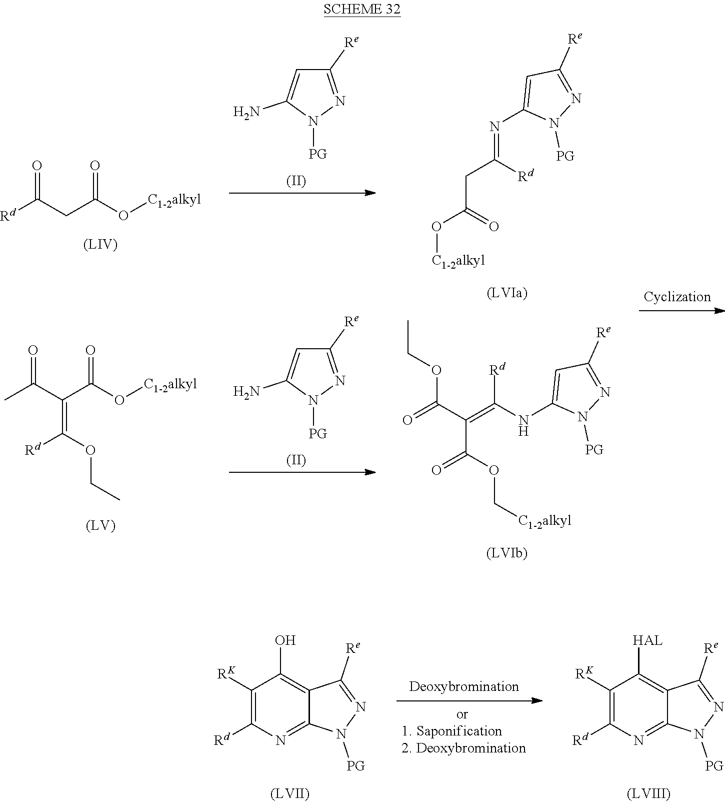

According to SCHEME 32, a commercially available or synthetically accessible compound of formula (LVIa) where R^{d} is C₁₋₃alkyl or C₃₋₆cycloalkyl, R^{e} is H or C₁₋₃alkyl, and PG is benzyl; is prepared condensation of a compound of formula (II), where R^{e} is H or C₁₋₃alkyl, and PG is benzyl; with a compound of formula (LIV) where R^{d} is C₁₋₃alkyl or C₃₋₆cycloalkyl; using a catalyst such asp-toluenesulfonic acid (TsOH) or acetic acid and the like; in a suitable solvent such as toluene and the like; at a temperature ranging from 70° C. to the reflux temperature of the solvent; for a period of about 14-24 h.

A compound of formula (LV), where R^{d} is C₁₋₃alkyl, is prepared via a Knoevenagel condensation between diethyl malonate and triethyl orthoacetate, in the presence of a Lewis acid such as zinc chloride and the like, at a temperature of 140° C. A compound of formula (LVIb) is prepared from the neat reaction of a compound of formula (LV) with formula (II), where R^{e} is H or C₁₋₃alkyl, and PG is benzyl; at a temperature of 120° C.

Thermal cyclization of a compound of formula (LVIa) or a compound of formula (LVIb) is achieved in a high-boiling solvent mixture such as Dowtherm™ A and the like, at a temperature of 275° C., for a period of about 1-6 h to provide a compound of formula (LVII), where R^{k} is H or CO₂Et. Deoxybromination of compound of formula (LVII), where R^{k} is H, is achieved using a brominating agent such as phosphorus oxybromide (POBr₃) and the like, in a mixture of solvents such as toluene and DMF, and the like, at a temperature ranging from 60 to 115° C., for a period of 1-2 h, to afford a compound of formula (LVIII), where R^{g} is H or C₁₋₃alkyl, R^{d} is C₁₋₃alkyl or C₃₋₆cycloalkyl, and PG is benzyl.

In an alternate method a compound of formula (LVIII), where R^{g} is H or C₁₋₃alkyl, R^{d} is C₁₋₃alkyl, R^{k} is CO₂H, and PG is benzyl, is prepared in two steps from a compound of formula (LVII), where R^{k} is CO₂Et. In a first step, saponification of a compound of formula (LVII), where R^{k} is CO₂Et, is achieved using a suitable base such as aqueous sodium hydroxide (NaOH), in a suitable solvent such as EtOH, and the like; at a temperature of 78° C., affords a compound of formula (LVII), where R^{k} is CO₂H. Deoxybromination of a compound of formula (LVII), where R^{k} is CO₂H is achieved employing methods known to one skilled in the art or as previously described, to afford a compound of formula (LVIII), where R^{g} is H or C₁₋₃alkyl, R^{d} is C₁₋₃alkyl, HAL is Br, and PG is benzyl

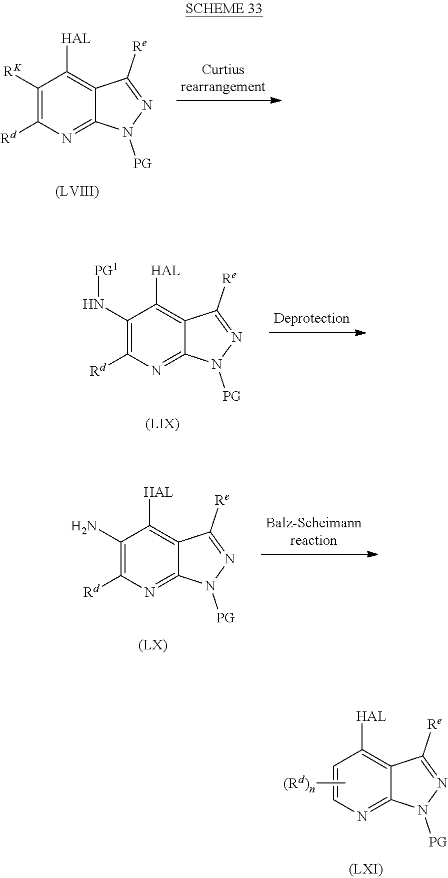

According to SCHEME 33, a compound of formula (LVIII), where HAL is Br, R^{k} is CO₂H, R^{g} is H or C₁₋₃alkyl, R^{d} is C₁₋₃alkyl, and PG is benzyl; is reacted in a Curtius rearrangement using a reagent such as diphenylphosphoryl azide (DPPA) and the like, in the presence of a base such as triethylamine (TEA) and the like, an alcohol such as tert-butanol (t-BuOH) and the like, in a solvent such as toluene and the like, and a temperature of 80-90° C.; to afford a compound of formula (LIX), where PG¹ is BOC. Deprotection of the BOC protecting group is achieved by reaction with an suitable acid such as TFA, HCl, and the like, in a suitable solvent such as DCM, dioxane, and the like, at temperatures ranging from 0° C. to 40° C. to afford a compound of formula (LX). A compound of formula (LX), is reacted under Balz-Scheimann reaction conditions, for example, diazotization of compound of formula (LX), using a tetrafluoroborate source such as tetrafluoroboric acid diethyl ether complex (HBF₄·Et₂O) and the like, with a diazotization reagent such as isopentyl nitrite and the like, in a solvent such as ACN and the like, at room temperature, followed by thermal decomposition of the diazotized intermediate, in an ionic liquid solvent such as [BMIM]BF₄ and the like, at a temperature of 200° C., affords a compound of formula (LXI), where HAL is Br, n is 2, R^{d} is independently selected from C₁₋₃alkyl and F, and R^{e} is C₁₋₃alkyl, and PG is benzyl

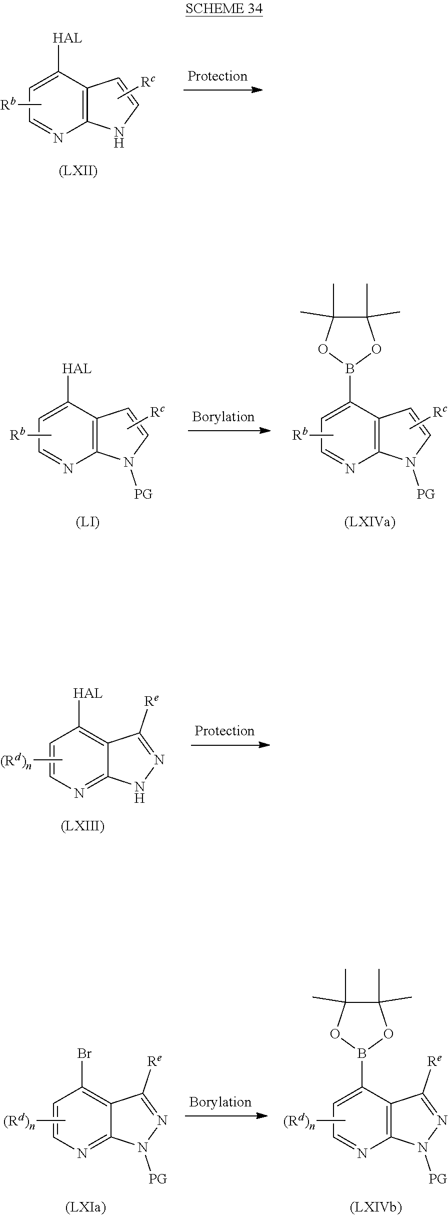

According to SCHEME 34, a commercially available or synthetically accessible compound of formula (LXII) where HAL is Br or Cl, R^{b} is H or OC₁₋₃alkyl, and R^{c} is H, C₁₋₃haloalkyl, or CN; is protected with a suitable nitrogen protecting group (PG) such as SEM (2-(trimethylsilyl)ethoxymethyl), tert-butyloxycarbonyl (BOC), Ts (toluenesulfonyl) or benzenesulfonyl, and the like, under conditions known to one skilled in the art, to provide a compound of formula (LI). A compound of formula (LXII) is protected with a SEM protecting group, employing conditions known to one skilled in the art, for example, by reaction of a compound of formula (LXII) with 2-chloromethoxyethyl)trimethylsilane, in the presence of a base such as NaH, and the like, in a suitable solvent such as DMF, and the like; at temperatures ranging from 0° C. to rt, to provide a compound of formula (LI), where PG is SEM. A compound of formula (LXII) is protected with a BOC protecting group, employing conditions known to one skilled in the art, for example, by reacting a compound of formula (LXII) with di-tert-butyl decarbonate (BOC anhydride) in the presence of a base such as Et₃N, and a catalyst such as DMAP, in a suitable solvent such as DCM, at temperatures ranging from 0° C. to rt, for a period of about 4-7 h to provide a compound of formula (LI), where PG is BOC. A compound of formula (LXII) is protected with a sulfonyl protecting group such as methanesulfonyl (Ms), benzenesulfonyl (Bs), toluenesulfonyl (Ts), nitrobenzenesulfonyl (Ns), and trifluoromethanesulfonyl (Tf); employing conditions known to one skilled in the art. For example, by reacting a compound of formula (LXII) is treated with a base such as Cs₂CO₃, and the like; 4-methylbenzenesulfonyl chloride; in a suitable solvent such as acetonitrile, and the like; to provide a compound of formula (LI), where PG is Ts. In a similar fashion, N-sulfonylation of a compound of formula (LXII), is achieved with benzenesulfonyl chloride, a base such as NaH, in a suitable solvent such as DMF, and the like; affords a compound of formula (LI), where PG is benzenesulfonyl (Bs).

An heteroaryl boron compound of formula (LXIVa) is prepared from a compound of formula (LI), where HAL is Br or Cl, R^{b} is H or OC₁₋₃alkyl, R^{c} is H, C₁₋₃haloalkyl, or CN, and PG is SEM (2-(trimethylsilyl)ethoxymethyl), Ts (toluenesulfonyl), benzenesulfonyl, or BOC (tert-butyloxycarbonyl; employing conditions known to one skilled in the art such as Miyaura borylation conditions. For example, a compound of formula (LI), where HAL is Br or Cl, R^{b} is H or OC₁₋₃alkyl, R^{c} is H, C₁₋₃haloalkyl, or CN, and PG is SEM (2-(trimethylsilyl)ethoxymethyl), Ts (toluenesulfonyl), benzenesulfonyl, or BOC (tert-butyloxycarbonyl) is treated with a transition metal catalyst such as 1,1′-bis(diphenylphosphino)ferrocene]dichloropalladium(II) (Pd(dppf)Cl₂), and the like; in a suitable solvent such as dimethylsulfoxide (DMSO) or 1,4-dioxane, and the like; and a base such as potassium acetate, and the like; and a boron source such as 4,4,4′,4′,5,5,5′,5′-octamethyl-2,2′-bi(1,3,2-dioxaborolane) (B₂Pin₂), bis(pinacolato)diboron, pinacol borane, and the like; at a temperature ranging from 80° C. to 100° C.; for a period of 2-8 h; to provide a compound of formula (LXIVa), where HAL is Br or Cl, R^{b} is H or OC₁₋₃alkyl, and R^{c} is H, C₁₋₃haloalkyl, or CN.

In a similar fashion, a compound of formula (LXIII), where HAL is Br, R^{e} is H, halo, and C₁₋₃alkyl, and R^{d} is independently selected from H, C₁₋₃alkyl, and cyclopropyl; is protected with a SEM protecting group, employing conditions known to one skilled in the art or as previously described to provide a compound of formula (LXIa), where PG is SEM. A compound of formula (LXIa) is borylated employing conditions known to one skilled in the art such as Miyaura borylation conditions, or as previously described, to provide a compound of formula (LXIVb)

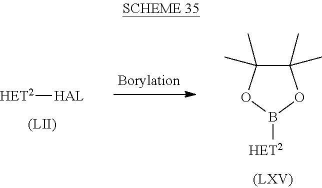

According to SCHEME 35, a commercially available or synthetically accessible compound of formula (LII), HET² is a 6-membered heteroaryl, fused 5,6- or fused 6,5-heteroaryl, or fused 6,6-heteroaryl ring optionally substituted with a suitable nitrogen protecting group, and HAL is Br or Cl; is borylated employing conditions known to one skilled in the art such as Miyaura borylation conditions, or as previously described, to provide a compound of formula (LXV)

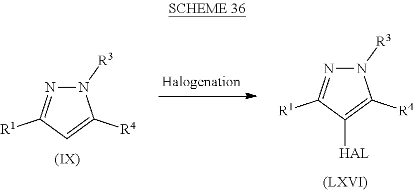

According to SCHEME 36, a compound of formula (LXVI), where R¹, R³, and R⁴ are as defined in Claim 1 and HAL is Cl, Br, or I, is prepared from a compound of formula (IX) (which encompasses compounds of formulas (XVII), (XXVI), and (XLIX)), using an electrophilic halogenating agent such as N-bromosuccinimide (NBS), N-iodosuccinimide (NIS), and the like; in a suitable solvent such as N,N-dimethylformamide (DMF), ACN, and the like

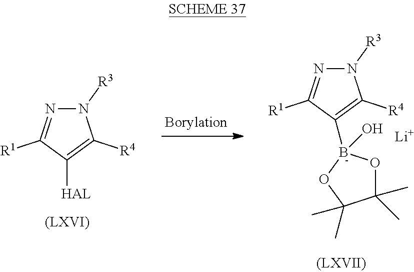

According to SCHEME 37, a compound of formula (LXVI) is borylated employing conditions known to one skilled in the art to provide a compound of formula (LXVII), where R¹, R³, and R⁴ are as described in claim 1. For example, 3-bromo-2-(5-fluoropyridin-2-yl)-6,6-dimethyl-6,7-dihydro-4H-pyrazolo[5,1-c][1,4]oxazine is borylated by treatment with a base such as n-butyllithium in the presence of 2-isopropoxy-4,4,5,5-tetramethyl-1,3,2-dioxaborolane in a solvent such as THF or toluene, and the like; at a temperature of −78° C., for 2 h, to afford lithium 2-(2-(5-fluoropyridin-2-yl)-6,6-dimethyl-6,7-dihydro-4H-pyrazolo[5,1-c][1,4]oxazin-3-yl)-2-hydroxy-4,4,5,5-tetramethyl-1,3,2-dioxaborolan-2-uide

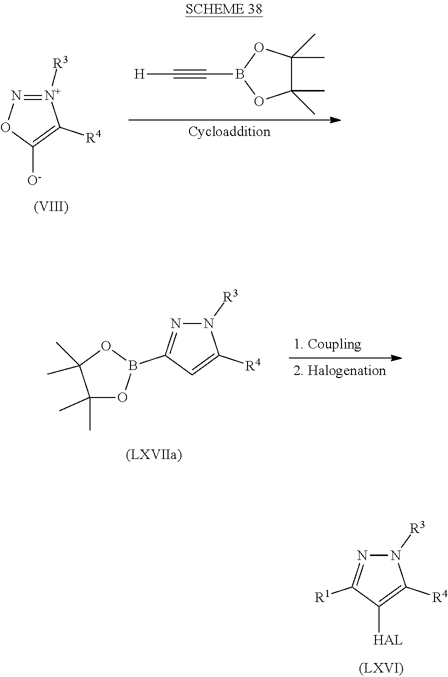

According to SCHEME 38, a compound of formula (VIII), where R³ and R⁴ are as defined in Claim 1, is reacted in a cycloaddition reaction with 2-ethynyl-4,4,5,5-tetramethyl-1,3,2-dioxaborolane, employing conditions previously described, to provide a compound of formula (LXVIIa). For example, 6,7-dihydro-4H-[1,2,3]oxadiazolo[4,3-c][1,4]oxazin-8-ium-3-olate is reacted with 2-ethynyl-4,4,5,5-tetramethyl-1,3,2-dioxaborolane in a solvent such as xylenes or toluene, and the like; at 150° C. for 16 h, to provide 2-(4,4,5,5-tetramethyl-1,3,2-dioxaborolan-2-yl)-6,7-dihydro-4H-pyrazolo[5,1-c][1,4]oxazine. A compound of formula (LXVI) is prepared in two steps from a compound of formula (LXVIIa). In a first step, a compound of formula (LXVIIa) is reacted in a metal mediated cross coupling reaction with a commercially available or synthetically accessible suitably substituted aryl or heteroaryl halide; in the presence of a palladium catalyst such as PdCl₂(dtbpf), Pd(PPh₃)₄, bis(triphenylphosphine)palladium(II)chloride (PdCl₂(PPh₃)₂), bis(diphenylphosphino)ferrocene]dichloropalladium(II) complex with dichloromethane, (2-dicyclohexylphosphino-2′,6′-diisopropoxy-1,1′-biphenyl)[2-(2′-amino-1,1′-biphenyl)]palladium(II) methanesulfonate (RuPhos Pd G3), [1,1′-bis(diphenylphosphino)ferrocene]dichloropalladium(II) (Pd(dppf)Cl₂), and the like; a base such as KF, K₃PO₄, aq. Na₂CO₃, Cs₂CO₃, and the like; in a suitable solvent such as 1,4-dioxane, DMF, acetonitrile (ACN), water, or a mixture thereof; at a temperature ranging from 60 to 120° C.; employing conventional or microwave heating; for a period of about 16 to 48 hours. Subsequent halogenation, employing methods previously described, affords a compound of formula (LXVI)

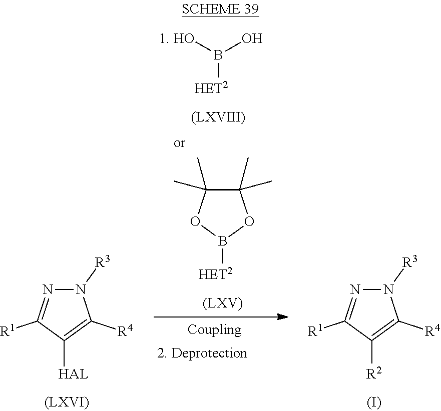

According to SCHEME 39, a compound of Formula (I) is prepared from a compound of formula (LXVI) (which includes compounds of formulas (XXXVI) and (XLV)), where R¹, R³ and R⁴ are as defined in Claim 1, and HAL is Br or I; and a commercially available or synthetically accessible suitably substituted monocyclic or bicyclic heteroaryl boronic acid of formula (LXVIII) or boronate ester of formula (LXV) (which also encompasses compounds of formulas (LXIVa), (LXIVb)) optionally containing a suitable nitrogen protecting group such as BOC, SEM, benzyl, tosyl, and the like; in a Pd-catalyzed cross-coupling reaction known to one skilled in the art. For example, reaction of a compound of formula (LXVI) with a compound of formula (LXVIII) or (LXV), employing Suzuki coupling conditions such as a palladium catalyst such as CataCXium® A Pd G3, XPhos Pd G3, Pd(dppf)Cl₂, and the like; optionally with a ligand such as dppf; a base such as K₃PO₄, K₂CO₃, aq. Na₂CO₃, Na₂CO₃, Cs₂CO₃, and the like; in a suitable solvent such as 1,4-dioxane, t-amyl alcohol, DMF, water, or a mixture thereof; at temperatures ranging from 60 to 130° C., employing microwave or conventional heating; for a period of 4 to 24 h. Deprotection of the nitrogen protecting group is achieved under conditions known to one skilled in the art provides a compound of Formula (I). For example, reaction with an acid such as TFA, HCl, and the like, optionally in a suitable solvent such as DCM, and the like, at room temperature; or reaction with a nucleophile such as fluoride, and the like, in a suitable solvent such as THF, and the like.

A compound of Formula (I), wherein the R² moiety is 1H-pyrazolo[3,4-b]pyridin-4-yl optionally substituted with F and CH₃; is chlorinated using a chlorinating reagent such as NCS and the like, optionally in the presence of a base such as sodium hydride and the like, in a suitable solvent such as DMF and the like; to provide a compound of Formula (I), where the 1H-pyrazolo[3,4-b]pyridin-4-yl is additionally substituted with C₁.

A compound of Formula (I), wherein the R² moiety is pyrazolo[1,5-a]pyridin-5-yl, is brominated or chlorinated employing conditions previously described or known to one skilled in the art, to provide a compound of Formula (I), wherein the R² moiety is pyrazolo[1,5-a]pyridin-5-yl substituted with Br or Cl. Iodination of a compound of Formula (I), wherein the R² moiety is pyrazolo[1,5-a]pyridin-5-yl is achieved using a strong base such as n-BuLi and the like; followed by addition of an iodinating agent such as diiodoethane and the like, in a suitable solvent such as THF and the like.

A compound of Formula (I), wherein the R² moiety is pyrazolo[1,5-a]pyridin-5-yl substituted with Br or Cl, is reacted under Suzuki coupling reaction conditions employing a boron reagent such as 2,4,6-trimethyl-1,3,5,2,4,6-trioxatriborinane; in the presence of a palladium catalyst such as Pd(dppf)Cl₁·CH₂Cl₂ and the like; a base such as K₂CO₃ and the like; in a suitable solvent such as dioxane and the like; to provide a compound of Formula (I), wherein the R² moiety is pyrazolo[1,5-a]pyridin-5-yl substituted with CH₃.

A compound of Formula (I), wherein the R² moiety is pyrazolo[1,5-a]pyridin-5-yl substituted with Br or Cl, is reacted under Buchwald coupling reaction conditions with diphenylmethanimine; in the presence of a base such as t-BuOK; a palladium catalyst such a Pd₂(dba)₃ and the like; optionally with a ligand such as rac-BINAP and the like; in a suitable solvent such as dioxane and the like; to provide a compound of Formula (I), wherein the R² moiety is pyrazolo[1,5-a]pyridin-5-yl substituted with NH₂.

A compound of Formula (I), wherein the R² moiety is pyrazolo[1,5-a]pyridin-5-yl is substituted with iodine, is reacted under Ullman coupling reaction conditions with tert-butyl carbamate; a copper reagent such as CuI and the like; in the presence of a base such as K₂CO₃ and the like; in a suitable solvent such as toluene and the like. Subsequent deprotection of the tert-butyl carbamate protecting group, employing conditions known to one skilled in the art, provides a compound of Formula (I), wherein the R² moiety is pyrazolo[1,5-a]pyridin-5-yl substituted with NH₂.

A compound of Formula (I), where R³ and R⁴ come together to form

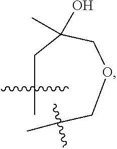 [SMILES: *COCC(C)(O)C*]

is alkylated with a suitable alkylating agent such as Mel, and the like; a suitable base such as Cs₂CO₃, and the like; employing conditions previously described to provide a compound of Formula (I), where R³ and R⁴ come together to form

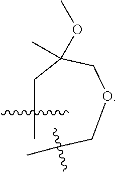 [SMILES: *COCC(C)(C*)OC]

A compound of Formula (I), where R³ and R⁴ come together to form

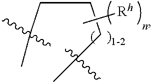

and R^{h} is as described in claim 1; is chlorinated employing methods known to one skilled in the art or as previously described to provide a compound of Formula (I), where R³ and R⁴ come together to form

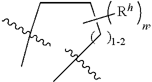

where one R^{h} member is Cl. A compound of Formula (I), where R³ and R⁴ come together to form

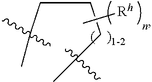

where one R^{h} member is Cl, is hydrolyzed using water and an appropriate organic solvent such as DMF and the like; to provide a compound of a compound of Formula (I), where R³ and R⁴ come together to form

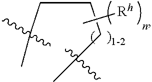

where one R^{h} member is OH

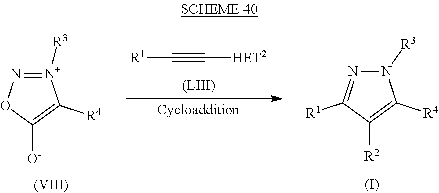

According to SCHEME 40, a compound of formula (LIII), where R¹ is a suitably substituted pyridyl as described in claim 1 and HET² is a suitably substituted pyridyl as defined in Claim 1, 1-methyl-1H-pyrazolo[4,3-b]pyridine or 1-((2-(trimethylsilyl)ethoxy)methyl)-1H-pyrrolo[2,3-b]pyridine-3-carbonitrile; is reacted with a compound of formula (VIII) such as 6-dimethyl-6,7-dihydro-4H-[1,2,3]oxadiazolo[4,3-c][1,4]oxazin-8-ium-3-olate; in a suitable solvent such as xylenes or toluene, and the like; at 150° C. for 16 h; to provide a compound of Formula (I).

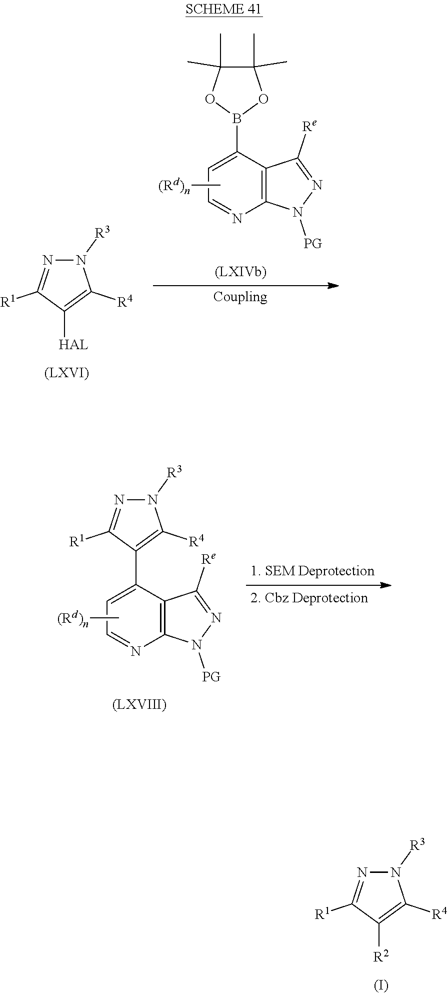

According to SCHEME 41, a compound of Formula (I), where R³ and R⁴ come together to form

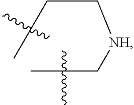 [SMILES: *CCNC*]

is prepared form a compound of formula (LXVI) and a compound of formula (LXIVb). For example, benzyl 2-(5-fluoropyridin-2-yl)-3-(6-methyl-1-((2-(trimethylsilyl)ethoxy)methyl)-1H-pyrazolo[3,4-b]pyridin-4-yl)-6,7-dihydropyrazolo[1,5-a]pyrazine-5(4H)-carboxylate is prepared from benzyl 3-bromo-2-(5-fluoropyridin-2-yl)-6,7-dihydropyrazolo[1,5-a]pyrazine-5(4H)-carboxylate and 6-methyl-4-(4,4,5,5-tetramethyl-1,3,2-dioxaborolan-2-yl)-1-((2-(trimethylsilyl)ethoxy)methyl)-1H-pyrazolo[3,4-b]pyridine employing Suzuki coupling conditions as previously described. 4-(2-(5-Fluoropyridin-2-yl)-4,5,6,7-tetrahydropyrazolo[1,5-a]pyrazin-3-yl)-6-methyl-1H-pyrazolo[3,4-b]pyridine is prepared in two steps from the cross-coupling product. In a first step, deprotection of the SEM protecting group is achieved reaction with an acid such as TFA, HCl, and the like, optionally in a suitable solvent such as DCM, and the like, at room temperature. In a second step, Cbz deprotection is achieved employing hydrogenation conditions known to one skilled in the art.

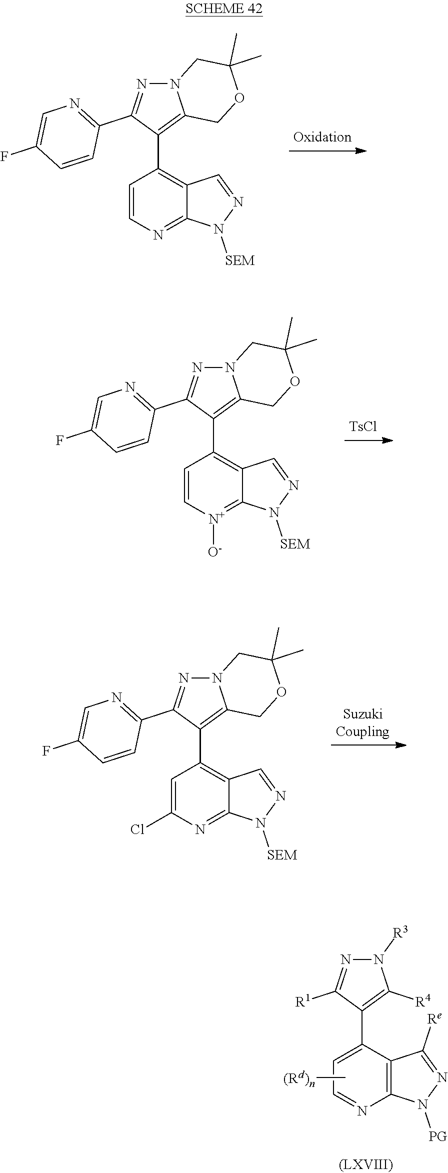

According to SCHEME 42, 2-(5-fluoropyridin-2-yl)-6,6-dimethyl-3-(1-((2-(trimethylsilyl)ethoxy)methyl)-1H-pyrazolo[3,4-b]pyridin-4-yl)-6,7-dihydro-4H-pyrazolo[5,1-c][1,4]oxazine is oxidized with an oxidant such as m-CPBA and the like, in a solvent such as DCM and the like, to provide 4-(2-(5-fluoropyridin-2-yl)-6,6-dimethyl-6,7-dihydro-4H-pyrazolo[5,1-c][1,4]oxazin-3-yl)-1-((2-(trimethylsilyl)ethoxy)methyl)-1H-pyrazolo[3,4-b]pyridine 7-oxide. Deoxychlorination of 4-(2-(5-fluoropyridin-2-yl)-6,6-dimethyl-6,7-dihydro-4H-pyrazolo[5,1-c][1,4]oxazin-3-yl)-1-((2-(trimethylsilyl)ethoxy)methyl)-1H-pyrazolo[3,4-b]pyridine 7-oxide is achieved using a reagent such as TsCl and the like, in a solvent such as DMF and the like, at a temperature of 85° C., to provide 3-(6-chloro-1-((2-(trimethylsilyl)ethoxy)methyl)-1H-pyrazolo[3,4-b]pyridin-4-yl)-2-(5-fluoropyridin-2-yl)-6,6-dimethyl-6,7-dihydro-4H-pyrazolo[5,1-c][1,4]oxazine. 3-(6-Chloro-1-((2-(trimethylsilyl)ethoxy)methyl)-1H-pyrazolo[3,4-b]pyridin-4-yl)-2-(5-fluoropyridin-2-yl)-6,6-dimethyl-6,7-dihydro-4H-pyrazolo[5,1-c][1,4]oxazine is reacted in a Suzuki coupling reaction with potassium trifluoro(3,3,3-trifluoropropyl)borate, employing methods previously described to afford a compound of formula (LXVIII), where R^{d} is 3,3,3-trifluoropropyl, R^{e} is H, and n is 1

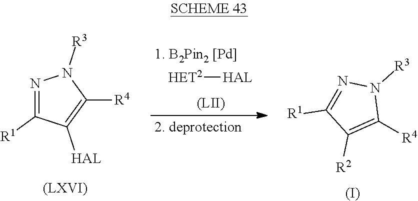

According to SCHEME 43, a compound of formula (LXVI), wherein R¹, R³, and R⁴ are as defined in Claim 1, and HAL is Cl, Br, or I is reacted with a compound of formula (LII), wherein HET² is methyl-1H-pyrazolo[3,4-d]pyrimidine, 5-fluoro-1H-pyrazolo[3,4-b]pyridin-4-yl, 4H-pyrrolo[1,2-b]pyrazol-3-yl, 6-methyl-1H-pyrazolo[3,4-d]pyrimidin-4-yl, 1H-pyrrolo[3,2-b]pyridin-7-yl, 1H-pyrazolo[4,3-b]pyridin-7-yl, and the like, wherein the HET² is protected with a suitable nitrogen protecting group such as SEM, THP, and the like; and HAL is Br; in a reductive cross-coupling reaction using a reagent such as B₂Pin₂ and the like; in the presence of a palladium catalyst such as Pd(tBu₃P)₂ and the like; a base such as K₃PO₄ and the like; in a solvent such as dioxane and the like; at a temperature of about 80 to 100° C. Deprotection of the nitrogen protecting group is achieved under conditions known to one skilled in the art, to provide compound of Formula (I).

Compounds of Formula (I) may be converted to their corresponding salts using methods known to one of ordinary skill in the art. For example, an amine of Formula (I) is treated with trifluoroacetic acid, HCl, or citric acid in a solvent such as Et₂O, CH₂Cl₂, THF, MeOH, chloroform, or isopropanol to provide the corresponding salt form. Alternately, trifluoroacetic acid or formic acid salts are obtained as a result of reverse phase HPLC purification conditions. Crystalline forms of pharmaceutically acceptable salts of compounds of Formula (I) may be obtained in crystalline form by recrystallization from polar solvents (including mixtures of polar solvents and aqueous mixtures of polar solvents) or from non-polar solvents (including mixtures of non-polar solvents).

Where the compounds according to this invention have at least one chiral center, they may accordingly exist as enantiomers. Where the compounds possess two or more chiral centers, they may additionally exist as diastereomers. It is to be understood that all such isomers and mixtures thereof are encompassed within the scope of the present invention.

Compounds prepared according to the schemes described above may be obtained as single forms, such as single enantiomers, by form-specific synthesis, or by resolution. Compounds prepared according to the schemes above may alternately be obtained as mixtures of various forms, such as racemic (1:1) or non-racemic (not 1:1) mixtures. Where racemic and non-racemic mixtures of enantiomers are obtained, single enantiomers may be isolated using conventional separation methods known to one of ordinary skill in the art, such as chiral chromatography, recrystallization, diastereomeric salt formation, derivatization into diastereomeric adducts, biotransformation, or enzymatic transformation. Where regioisomeric or diastereomeric mixtures are obtained, as applicable, single isomers may be separated using conventional methods such as chromatography or crystallization.

The following specific examples are provided to further illustrate the invention and various preferred embodiments.

### EXAMPLES

In obtaining the compounds described in the examples below and the corresponding analytical data, the following experimental and analytical protocols were followed unless otherwise indicated.

Unless otherwise stated, reaction mixtures were magnetically stirred at room temperature (rt) under a nitrogen atmosphere. Where solutions were “dried,” they were generally dried over a drying agent such as Na₂SO₄ or MgSO₄. Where mixtures, solutions, and extracts were “concentrated”, they were typically concentrated on a rotary evaporator under reduced pressure. Reactions under microwave irradiation conditions were carried out in a Biotage Initiator or CEM (Microwave Reactor) Discover instrument.

Normal-phase silica gel chromatography (FCC) was performed on silica gel (SiO₂) using prepacked cartridges.

Preparative reverse-phase high performance liquid chromatography (RP HPLC) was performed on either:

### Reverse Phase Preparative HPLC Method A:

Welch Xtimate C18 column (5 m, 150 mm×25 mm): eluent: 50% to 80% (v/v) CH₃CN and H₂O with 0.225% HCOOH.

Reverse Phase Preparative HPLC Method B:

Boston Uni C18 column (5 m, 150 mm×40 mm): eluent: 70% to 100% (v/v) CH₃CN and H₂O with 0.225% HCOOH.

### Reverse Phase Preparative HPLC Method C:

An Agilent HPLC; Waters XBridge C18 column (5 m, 50×100 mm) eluent: 5-90% MeCN/20 mM NH₄OH over 15 min, flow rate 80 mL/min.

### Reverse Phase Preparative HPLC Method D:

An ACCQ Prep HPLC, XBridge C18 OBD column (5 μM, 50×100): eluent: 0-100% MeCN/water, 20 mM NH₄OH modifier.

### Reverse Phase Preparative HPLC Method E:

Welch Xtimate C18 column (5 μM, 150×25 mm): eluent: 32% to 62% (v/v) CH₃CN and H₂O with 0.04% NH₃H₂O.

### Reverse Phase Preparative HPLC Method F:

Phenomenex Gemini NX-C18 column (3 m, 75 mm×30 mm): eluent: 33% to 63% (v/v) CH₃CN and H₂O with 0.05% NH₃+10 mM NH₄HCO₃; or eluent: 21% to 51% (v/v) CH₃CN and H₂O with 0.05% NH₃+10 mM NH₄HCO₃; or eluent: 35% to 65% (v/v) CH₃CN and H₂O with 0.05% NH₃+10 mM NH₄HCO₃; or eluent: 30% to 30% (v/v) CH₃CN and H₂O with 0.05% NH₃+10 mM NH₄HCO₃.

### Reverse Phase Preparative HPLC Method G:

Boston Prime C18 column (5 m, 150 mm×30 mm): eluent: 35% to 65% (v/v) CH₃CN and H₂O with 0.05% NH₃+10 mM NH₄HCO₃; or eluent: 40% to 70% (v/v) CH₃CN and H₂O with 0.05% NH₃+10 mM NH₄HCO₃; or eluent: 70% to 100% (v/v) CH₃CN and H₂O with 0.05% NH₃+10 mM NH₄HCO₃; or eluent: 30% to 60% (v/v) CH₃CN and H₂O with 0.05% NH₃+10 mM NH₄HCO₃.

### Reverse Phase Preparative HPLC Method H:

An ACCQ Prep HPLC; with an XBridge C18 OBD column (5 μM, 50×100), eluent 20-80% MeCN:H₂O w/0.05% TFA.

### Reverse Phase Preparative HPLC Method I:

Boston Green ODS column (5 μM, 150 mm×30 mm); eluent: 20% to 50% (v/v) CH₃CN and H₂O with 0.25% HCOOH.

Preparative supercritical fluid high performance liquid chromatography (SFC) was performed either on a Jasco preparative SFC system or a Waters Prep SFC 150 AP system The separations were conducted at 100 to 150 bar with a flow rate ranging from 40 to 60 mL/min. The column was heated to 35 to 40° C.

### SFC Method A:

DAICEL CHIRALCEL® OD column: (10 m, 250 mm×50 mm): isocratic elution: EtOH (containing 0.1% of 25% aq. NH₃): supercritical CO₂, 20%: 80% to 20%: 80% (v/v) or isocratic elution: EtOH (containing 0.1% of 25% aq. NH₃): supercritical CO₂, 30%: 70% to 30%: 70% (v/v).

### SFC Method B:

DAICEL CHIRALCEL® OD-H column: (5 m, 250 mm×30 mm): isocratic elution: EtOH (containing 0.1% of 25% aq. NH₃): supercritical CO₂, 20%: 80% to 20%: 80% (v/v) or isocratic elution: EtOH (containing 0.1% of 25% aq. NH₃): supercritical CO₂, 45%: 55% to 45%: 55% (v/v).

### SFC Method C:

DAICEL CHIRALPAK® AD-H column: (10 μm, 250 mm×30 mm) or (5 μm, 250 mm×30 mm): isocratic elution: EtOH (containing 0.1% of 25% aq. NH₃): supercritical CO₂, 45%: 55% to 45%: 55% (v/v) or isocratic elution: EtOH (containing 0.1% of 25% aq. NH₃): supercritical CO₂, 40%: 60% to 40%: 60% (v/v) or isocratic elution: IPA (containing 0.1% of 25% aq. NH₃): supercritical CO₂, 40%: 60% to 40%: 60% (v/v).

### SFC Method D:

DAICEL CHIRALPAK® AD column: (10 μm, 250 mm×30 mm) or (5 μm, 250 mm×30 mm): isocratic elution: EtOH (containing 0.1% of 25% aq. NH₃): supercritical CO₂, 45%: 55% to 45%: 55% (v/v)) or eluent: EtOH (containing 0.1% of 25% aq. NH₃): supercritical CO₂, 45% to 45% (v/v) or isocratic elution: EtOH (containing 0.1% of 25% aq. NH₃): supercritical CO₂, 55%: 45% to 55%: 45% (v/v) or isocratic elution: IPA (containing 0.1% of 25% aq. NH₃): supercritical CO₂, 55%: 45% to 55%: 45% (v/v) or isocratic elution: EtOH (containing 0.1% of 25% aq. NH₃): supercritical CO₂, 30%: 70% to 30%: 70% (v/v) or isocratic elution: EtOH (containing 0.1% of 25% aq. NH₃): supercritical CO₂, 40%: 60% to 40%: 60% (v/v) or isocratic elution: EtOH (containing 0.1% of 25% aq. NH₃): supercritical CO₂, 50%: 50% to 50%: 50% (v/v).

### SFC Method E:

DAICEL CHIRALCEL® OJ column (10 μm, 250 mm×30 mm): eluent: EtOH (containing 0.1% of 25% aq. NH₃): supercritical CO₂, 25% to 25% (v/v) or isocratic elution: EtOH (containing 0.1% of 25% aq. NH₃): supercritical CO₂, 15%: 85% to 15%: 85% (v/v).

### SFC Method F:

DAICEL CHIRALCEL®OJ-H column (5 μm, 250 mm×30 mm): isocratic elution: EtOH (containing 0.1% of 25% aq. NH₃): supercritical CO₂, 25%: 75% to 25%: 75% (v/v) or isocratic elution: EtOH (containing 0.1% of 25% aq. NH₃): supercritical CO₂, 30%: 70% to 30%: 70% (v/v) or isocratic elution: IPA (containing 0.1% of 25% aq. NH₃): supercritical CO₂, 65%: 35% to 65%: 35% (v/v).

### SFC Method G:

DAICEL CHIRALPAK® IG column: (10 μm, 250 mm×30 mm): isocratic elution: EtOH (containing 0.1% of 25% aq. NH₃): supercritical CO₂, 55%: 45% to 55%: 45% (v/v).

### SFC Method H:

DAICEL CHIRALPAK® IC column (5 μm, 250 mm×30 mm): isocratic elution: MeOH (containing 0.1% of 25% aq. NH₃): supercritical CO₂, 35%: 65% to 35%: 65% (v/v).

### SFC Method I:

Chiralcel OZ-H column (5 μm 250×21 mm): Mobile phase: 25% methanol with 0.2% triethylamine, 75% CO₂, flow rate 42 mL/min, monitor at 220 nm

Photochemical reactions were conducted in a PennOC Photoreactor M1 (450 nm wavelength, 100% LED power, 100% fan power, and 750 rpm stirring).

Mass spectra (MS) were obtained on an Agilent series 1100 MSD using electrospray ionization (ESI) in positive mode unless otherwise indicated. Calculated (calcd.) mass corresponds to the exact mass.

Analytical LCMS was obtained on an Agilent 1260 Series using an ACE Excel 3 C18 column (3 m, 2.1×35 mm, T=50 C). Mobile phase A: 0.05% TFA in H₂O and mobile phase B: 100% acetonitrile. Method gradient starts at 5% B to 100% B in 2.2 minutes at flow rate 1.0 mL/min. MS detector is an Agilent G6125B MSD set in positive mode.

Nuclear magnetic resonance (NMR) spectra were obtained on Bruker Avance Neo spectrometers. Definitions for multiplicity are as follows: s=singlet, d=doublet, t=triplet, q=quartet, m=multiplet, br=broad, dd=doublet of doublets, dt=doublet of triplets, td=triplet of doublets. It will be understood that for compounds comprising an exchangeable proton, said proton may or may not be visible on an NMR spectrum depending on the choice of solvent used for running the NMR spectrum and the concentration of the compound in the solution.

Chemical names were generated using ChemDraw Ultra 17.1 (CambridgeSoft Corp., Cambridge, MA) or OEMetaChem V1.4.0.4 (Open Eye).

Compounds designated as *R or *S are enantiopure compounds where the absolute configuration was not determined.

### Intermediate 1: 5,6-Dihydro-4H-pyrrolo[1,2-c][1,2,3]oxadiazol-7-ium-3-olate

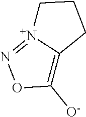

To a solution of L-proline (506 mg, 4.4 mmol) in H₂O (1 mL) was added sodium nitrite (455 mg, 6.6 mmol) followed by HCl (37% in H₂O, 0.77 mL, 9.2 mmol). The reaction was stirred at RT for 12 hours then extracted with EtOAc (3×5 mL). The combined organics were dried (Na₂SO₄), filtered, and concentrated. The residue was dissolved in MeCN (4.4 mL) and trifluoroacetic anhydride (0.92 mL, 6.6 mmol) was added. The reaction was stirred at RT for 2 hours then quenched with potassium carbonate (1.2 g, 8.8 mmol). The reaction was condensed then water (10 mL) was added and the resulting mixture was extracted with 4:1 CH₂Cl₂:isopropanol (5×25 mL). The combined organics were dried (Na₂SO₄), filtered, and condensed to afford the title compound (310 mg, 56%). MS (ESI): mass calcd. for C₅H₆N₂O₂, 126.0; m/z found, 127.1 [M+H]⁺. ¹H NMR (500 MHz, CD₃OD) δ 4.54-4.47 (m, 2H), 2.87-2.80 (m, 2H), 2.80-2.69 (m, 2H).

### Intermediate 2: 6,7-Dihydro-4H-[1,2,3]oxadiazolo[4,3-c][1,4]oxazin-8-ium-3-olate

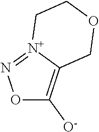

Step A: 4-Nitrosomorpholine-3-carboxylic acid. To morpholine-3-carboxylic acid (361 mg, 2.8 mmol) was added water (0.64 mL), sodium nitrite (285 mg, 4.1 mmol) and HCl (37% in H₂O, 0.46 mL). The reaction mixture was stirred at room temperature for 16 hours then water was added. The aqueous phase was extracted 3 times with a mixture of 20% IPA in CHCl₃. The combined organic layers were dried over MgSO₄, filtered and evaporated. The material was used as is in the next step without any further purification. MS (ESI): mass calcd. for C₅H₈N₂O₄, 160.1; m/z found, 161.1 [M+H]⁺.

Step B: 6,7-Dihydro-4H-[1,2,3]oxadiazolo[4,3-c][1,4]oxazin-8-ium-3-olate. To a solution of 4-nitrosomorpholine-3-carboxylic acid in acetonitrile (2.8 mL) was added trifluoroacetic anhydride (0.58 mL, 4.1 mmol) and the reaction mixture was stirred at room temperature for 2 hours. Potassium carbonate (762 mg, 5.5 mmol) was then added followed by water. The aqueous phase was extracted 4 times with a mixture of 20% IPA in CHCl₃. The combined organic layers were dried over MgSO₄, filtered, and evaporated to afford the title compound (248 mg, 63% yield). The material was used as is in the next step without any further purification. MS (ESI): mass calcd. for C₅H₆N₂O₃, 142.0; m/z found, 143.1 [M+H]⁺.

### Intermediate 3: (S)-6-Methyl-5,6-dihydro-4H-pyrrolo[1,2-c][1,2,3]oxadiazol-7-ium-3-olate

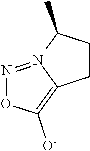

Step A: (2S,5S)-5-Methylpyrrolidine-2-carboxylic acid hydrochloride. HCl/1,4-dioxane (30 mL, 4M) was added dropwise to a 100 mL round-bottomed flask containing (2S,5S)-1-(tert-butoxycarbonyl)-5-methylpyrrolidine-2-carboxylic acid (5.00 g, 21.8 mmol). The resultant mixture was stirred at room-temperature for 3 hours. The mixture was concentrated to dryness under reduced pressure to afford the title compound (4 g) as a white solid, which was used in the next step without further purification. ¹H NMR (400 MHz, DMSO-d₆) δ 10.22 (br s, 1H), 8.52 (br s, 1H), 4.41-4.23 (m, 1H), 3.70-3.51 (m, 1H), 2.35-2.19 (m, 1H), 2.16-2.00 (m, 2H), 1.61-1.49 (m, 1H), 1.33-1.29 (m, J=6.8 Hz, 3H).

Step B: (S)-6-Methyl-5,6-dihydro-4H-pyrrolo[1,2-c][1,2,3]oxadiazol-7-ium-3-olate. The title compound was prepared in a manner analogous to Intermediate 2, Step A-B except using (2S,5S)-5-methylpyrrolidine-2-carboxylic acid instead of morpholine-3-carboxylic acid and AcOH instead of HCl in Step A and THF instead of ACN in Step B.

### Intermediate 4: (R)-6-Methyl-5,6-dihydro-4H-pyrrolo[1,2-c][1,2,3]oxadiazol-7-ium-3-olate

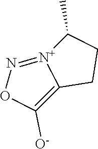

The title compound was made in a manner analogous to Intermediate 3, Steps A-B, except using (2R,5R)-1-(tert-butoxycarbonyl)-5-methylpyrrolidine-2-carboxylic acid instead of (2S,5S)-1-(tert-butoxycarbonyl)-5-methylpyrrolidine-2-carboxylic acid in Step A. ¹H NMR (400 MHz, CDCl₃): δ 4.85-4.65 (m, 1H), 2.97-2.77 (m, 3H), 2.43-2.31 (m, 1H), 1.68 (d, J=6.6 Hz, 3H).

### Intermediate 5: (S)-5-Fluoro-5,6-dihydro-4H-pyrrolo[1,2-c][1,2,3]oxadiazol-7-ium-3-olate

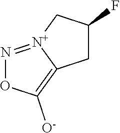

The title compound was prepared in a manner analogous to Intermediate 1, except using (2S,4S)-4-fluoropyrrolidine-2-carboxylic acid instead of L-proline. MS (ESI): mass calcd. for C₅H₅FN₂O₂, 144.0; m/z found, 145.1 [M+H]⁺.

### Intermediate 6: Racemic (3bS,4aR)-3b,4,4a,5-tetrahydrocyclopropa[3,4]pyrrolo[1,2-c][1,2,3]oxadiazol-6-ium-3-olate

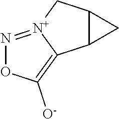

The title compound was prepared in a manner analogous to Intermediate 1, except using racemic (1S,5R)-3-azabicyclo[3.1.0]hexane-2-carboxylic acid instead of L-proline. MS (ESI): mass calcd. for C₆H₆N₂O₂, 138.0; m/z found, 139.1 [M+H]⁺.

### Intermediate 7: (4aR,5aR)-4,4a,5,5a-Tetrahydrocyclopropa[4,5]pyrrolo[1,2-c][1,2,3]oxadiazol-6-ium-3-olate

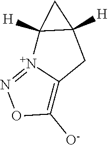

A solution of (1R,3S,5R)-2-(tert-butoxycarbonyl)-2-azabicyclo[3.1.0]hexane-3-carboxylic acid (1.00 g, 4.40 mmol) in TFA (10 mL) was stirred at r.t. for 30 minutes and then concentrated. The residue was dissolved in a mixture of water (5 mL) and aq. HCl (37%, 0.75 mL). Sodium nitrite (455 mg, 6.60 mmol) was added in one portion, and the reaction mixture was stirred at r.t. for 2 hours, then diluted with water and extracted 3× with a 4:1 mixture of chloroform/isopropanol. The combined organic layers were dried (MgSO₄), concentrated, and left under high vacuum overnight. The residue was dissolved in MeCN (15 mL), trifluoroacetic anhydride (0.92 mL, 6.6 mmol) was added dropwise, and the reaction mixture was stirred at r.t. for two hours. The mixture was quenched by added K₂CO₃ (3.0 g, 22 mmol) and stirred at r.t. for 20 minutes, then concentrated to remove solvent, and partitioned between water and 4:1 DCM/isopropanol. The aqueous layer was extracted 3× with 4:1 DCM/isopropanol and the combined organics were dried (MgSO₄) and concentrated to obtain 346 mg (2.51 mmol, 57% yield) of the title compound, which was used directly subsequent transformation without purification. MS (ESI): mass calcd. for C₆H₆N₂O₂, 138.0; m/z found, 139.1 [M+H]⁺.

### Intermediate 8: (4aS,5aS)-4,4a,5,5a-Tetrahydrocyclopropa[4,5]pyrrolo[1,2-c][1,2,3]oxadiazol-6-ium-3-olate

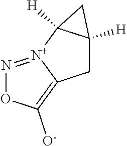

The title compound was prepared in a manner analogous to Intermediate 7, except using (1S,3S,5S)-2-(tert-butoxycarbonyl)-2-azabicyclo[3.1.0]hexane-3-carboxylic acid instead of (1R,3S,5R)-2-(tert-butoxycarbonyl)-2-azabicyclo[3.1.0]hexane-3-carboxylic acid. MS (ESI): mass calcd. for C₆H₆N₂O₂, 138.0; m/z found, 139.1 [M+H]⁺.

### Intermediate 9: 4,5,6,7-Tetrahydro-[1,2,3]oxadiazolo[3,4-a]pyridin-8-ium-3-olate

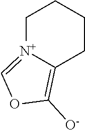

The title compound was prepared in a manner analogous to Intermediate 2, Steps A-B except using piperidine-2-carboxylic acid instead of morpholine-3-carboxylic acid in Step A. MS (ESI): mass calcd. for C₆H₈N₂O₂, 140.1; m/z found, 141.1 [M+H]⁺.

### Intermediate 10: Racemic 7-Methyl-4,5,6,7-tetrahydro-[1,2,3]oxadiazolo[3,4-a]pyridin-8-ium-3-olate

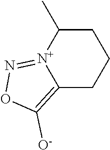

The title compound was prepared in a manner analogous to Intermediate 2, Steps A-B except using 6-methylpiperidine-2-carboxylic acid instead of morpholine-3-carboxylic acid in Step A. MS (ESI): mass calcd. for C₇H₁₀N₂O₂, 154.1; m/z found, 154.8 [M+H]⁺. ¹H NMR (400 MHz, CDCl₃): δ 4.44 (qd, J=6.6, 13.1 Hz, 1H), 2.73-2.47 (m, 2H), 2.30-2.16 (m, 1H), 2.11-1.97 (m, 1H), 1.86-1.75 (m, 2H), 1.67 (d, J=6.6 Hz, 3H).

### Intermediate 11: 6,6-Difluoro-4,5,6,7-tetrahydro-[1,2,3]oxadiazolo[3,4-a]pyridin-8-ium-3-olate

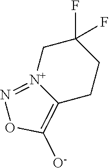 [SMILES: [O-]c1on[n+]2c1CCC(F)(F)C2]

The title compound was prepared in a manner analogous to Intermediate 2, Steps A-B except using 5,5-difluoropiperidine-2-carboxylic acid instead of morpholine-3-carboxylic acid in Step A. MS (ESI): mass calcd. for C₆H₆F₂N₂O₂, 176.0; m/z found, 177.0 [M+H]⁺. ¹H NMR (400 MHz, DMSO-d₆) δ 5.04 (t, J=11.9 Hz, 2H), 2.82-2.66 (m, 2H), 2.48-2.38 (m, 2H).

### Intermediate 12: 5,5-Difluoro-4,5,6,7-tetrahydro-[1,2,3]oxadiazolo[3,4-a]pyridin-8-ium-3-olate

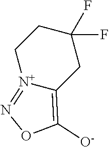 [SMILES: [O-]c1on[n+]2c1CC(F)(F)CC2]

Step A: 4,4-Difluoropiperidine-2-carboxylic acid. The title compound was made in a manner analogous to Intermediate 3, except using 1-(tert-butoxycarbonyl)-4,4-difluoropiperidine-2-carboxylic acid instead of (2S,5S)-1-(tert-butoxycarbonyl)-5-methylpyrrolidine-2-carboxylic acid in Step A. The title compound was used in the subsequent step without further purification.

Step B: 5,5-Difluoro-4,5,6,7-tetrahydro-[1,2,3]oxadiazolo[3,4-a]pyridin-8-ium-3-olate. The title compound was prepared in a manner analogous to Intermediate 2, Steps A-B except using 4,4-difluoropiperidine-2-carboxylic acid instead of morpholine-3-carboxylic acid in Step A. MS (ESI): mass calcd. for C₆H₆F₂N₂O₂, 176.0; m/z found, 176.8 [M+H]⁺. ¹H NMR (400 MHz, CDCl₃): δ 4.60-4.49 (m, 2H), 3.21 (t, J=13.4 Hz, 2H), 2.62 (tt, J=6.3, 12.1 Hz, 2H).

### Intermediate 13: 5,5-Dimethyl-4,5,6,7-tetrahydro-[1,2,3]oxadiazolo[3,4-a]pyridin-8-ium-3-olate

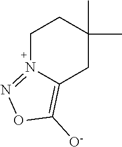

Step A: 4,4-Dimethylcyclohexan-1-one oxime. 4,4-Dimethylcyclohexanone (16.0 g, 127 mmol), NH₂OH·HCl (11.5 g, 165 mmol), Na₂CO₃ (17.6 g, 166 mmol), H₂O (80 mL), and EtOH (80 mL) were added to a 250 mL three-necked round-bottomed flask, equipped with mechanical stirrer, condensing tube, and thermometer. The resultant mixture was heated at 100° C. for 2 hours. The reaction mixture was cooled to room-temperature. The majority of the EtOH was removed under reduced pressure, the residue diluted with H₂O (80 mL), and the resultant mixture extracted with ethyl acetate (200 mL×3). The combined organic extracts were washed with brine (150 mL), dried over anhydrous Na₂SO₄, filtered, and concentrated to dryness under reduced pressure to give the title compound (17 g, 95%) as a white solid, which was used in the next step without further purification. ¹H NMR (400 MHz, DMSO-d₆) δ 10.14 (s, 1H), 2.41-2.34 (m, 2H), 2.17-2.09 (m, 2H), 1.40-1.34 (m, 2H), 1.30 (t, J=6.7 Hz, 2H), 0.95 (s, 6H).

Step B: 3,3-Dichloro-5,5-dimethylazepan-2-one. A solution consisting of 4,4-dimethylcyclohexanone oxime (8.0 g, 57 mmol) and xylenes (100 mL) was added dropwise to a stirred slurry mixture consisting of PCl₅ (35.4 g, 170 mmol) and xylene (300 mL) at 35° C. The resultant mixture was heated at 90° C. for 16 hours. The reaction mixture was cooled to room-temperature, poured it into sat. Na₂CO₃ (450 mL) and extracted with ethyl acetate (400 mL×3). The combined organic extracts were washed with brine (200 mL), dried over anhydrous Na₂SO₄, filtered, and concentrated to dryness under reduced pressure. The resulting residue was triturated with petroleum ether (100 mL) and the suspension isolated via filtration. The filter cake was washed with petroleum ether (100 mL). The resulting filtrate was concentrated under reduced pressure to afford the title compound (7.8 g, 66%) as a grey solid, which was used in the next step without further purification. ¹H NMR (400 MHz, DMSO-d₆) δ 8.36 (br s, 1H), 3.25-3.14 (m, 2H), 2.55 (s, 2H), 1.51-1.45 (m, 2H), 1.09 (s, 6H).

Step C: 3-Chloro-5,5-dimethylazepan-2-one. 3,3-Dichloro-5,5-dimethylazepan-2-one (1.0 g, 4.8 mmol), glacial acetic acid (30 mL), and wet Pd/C (500 mg, 10 wt. %) were added to a 100 mL hydrogenation bottle. The resultant mixture was stirred under H₂ (50 psi) at room-temperature for 15 hours. The suspension was filtered through a pad of Celite® and the pad washed with ethyl acetate (30 mL). The filtrate was concentrated to dryness under reduced pressure. Dichloromethane (60 mL) and aqueous saturated NaHCO₃(60 mL) were added to the residue and the mixture was stirred for 10 mins. The organic layer was separated, dried over anhydrous Na₂SO₄, filtered, and concentrated to dryness under reduced pressure to give the title compound (800 mg, 96%) as a yellow solid, which was used in the next step without further purification. ¹H NMR (400 MHz, DMSO-d₆) δ 7.12 (br s, 1H), 4.73 (dd, J=1.5, 11.8 Hz, 1H), 3.39-3.24 (m, 1H), 3.22-3.11 (m, 1H), 2.07-1.86 (m, 2H), 1.47-1.39 (m, 2H), 1.11 (s, 3H), 1.01 (s, 3H).

Step D: 1-((Benzyloxy)carbonyl)-4,4-dimethylpiperidine-2-carboxylic acid. 3-Chloro-5,5-dimethylazepan-2-one (800 mg, crude), Ba(OH)₂·8 H₂O (1.8 g, 5.7 mmol) and H₂O (30 mL) were added to a 100 mL three-necked round-bottomed flask equipped with mechanical stirrer, condensing tube, and thermometer. The resultant mixture was heated at 115° C. for 2 hours. The reaction mixture was cooled to room-temperature. The mixture was treated with a solution consisting of CbzCl (1.1 g, 6.4 mmol) and THF (30 mL). The resultant mixture was stirred at room-temperature for 16 hours. The pH was adjusted with 1M HCl to pH=3 and extracted with dichloromethane (60 mL×3). The combined organic extracts were washed with brine (50 mL), dried over anhydrous Na₂SO₄, filtered, and concentrated to dryness under reduced pressure to give the title compound (1.5 g, crude) was a yellow oil, which was used in the next step without further purification. ¹H NMR (400 MHz, CDCl₃): δ 7.34-7.28 (m, 5H), 5.19-5.12 (m, 2H), 4.09-3.91 (m, 2H), 3.33-3.13 (m, 1H), 2.13-2.06 (m, 1H), 1.66 (dd, J=7.4, 14.2 Hz, 1H), 1.45-1.34 (m, 2H), 0.96 (s, 3H), 0.92 (s, 3H).

Step E: 4,4-Dimethylpiperidine-2-carboxylic acid. 1-((Benzyloxy)carbonyl)-4,4-dimethylpiperidine-2-carboxylic acid (1.5 g, crude), methanol (30 mL), and wet Pd/C (500 mg, 10 wt. %) were added to a 100 mL hydrogenation bottle. The resultant mixture was stirred under H₂ (50 psi) at room-temperature for 15 hours. The suspension was filtered through a pad of Celite® and the pad washed with methanol (50 mL). The filtrate was concentrated to dryness under reduced pressure to afford the title product (1 g, crude) as a colorless oil, which was used in the next step without further purification.

Step F: 5,5-Dimethyl-4,5,6,7-tetrahydro-[1,2,3]oxadiazolo[3,4-a]pyridin-8-ium-3-olate. The title compound was prepared in a manner analogous to Intermediate 2, Step A-B expect using 4,4-dimethylpiperidine-2-carboxylic acid instead of morpholine-3-carboxylic acid in Step A and THF instead of CH₃CN in Step B. MS (ESI): mass calcd. for C₈H₁₂N₂O₂, 168.1; m/z found, 169.2 [M+H]⁺. ¹H NMR (400 MHz, CDCl₃): δ 4.29 (t, J=6.5 Hz, 2H), 2.43 (s, 2H), 1.90 (t, J=6.4 Hz, 2H), 1.14 (s, 6H).

### Intermediate 14: Racemic (5aR,6aS)-5,5a,6,6a-Tetrahydro-4H-cyclopropa[e][1,2,3]oxadiazolo[3,4-a]pyridin-7-ium-3-olate

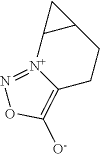 [SMILES: [O-]c1on[n+]2c1CCC1CC12]

The title compound was prepared in a manner analogous to Intermediate 7, except using 2-(tert-butoxycarbonyl)-2-azabicyclo[4.1.0]heptane-3-carboxylic acid instead of (1R,3S,5R)-2-(tert-butoxycarbonyl)-2-azabicyclo[3.1.0]hexane-3-carboxylic acid, 6 equivalents of trifluoroacetic anhydride were used instead of 1.5, and the acetonitrile solution was stirred overnight instead of for two hours. MS (ESI): mass calcd. for C₇H₈N₂O₂, 152.1; m/z found, 153.1 [M+H]⁺.

### Intermediate 15: Racemic (5aR,6aS)-6,6-Difluoro-5,5a,6,6a-tetrahydro-4H-cyclopropa[e][1,2,3]oxadiazolo[3,4-a]pyridin-7-ium-3-olate

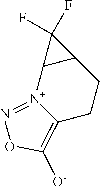 [SMILES: [O-]c1on[n+]2c1CCC1C2C1(F)F]

The title compound was prepared in a manner analogous to Intermediate 7, except using 2-(tert-butoxycarbonyl)-7,7-difluoro-2-azabicyclo[4.1.0]heptane-3-carboxylic acid instead of (1R,3S,5R)-2-(tert-butoxycarbonyl)-2-azabicyclo[3.1.0]hexane-3-carboxylic acid, 6 equivalents of trifluoroacetic anhydride were used instead of 1.5, and the acetonitrile solution was stirred overnight instead of for two hours. MS (ESI): mass calcd. for C₇H₆F₂N₂O₂, 188.0; m/z found, 189.1 [M+H]⁺.

### Intermediate 16: (R)-4-Methyl-6,7-dihydro-4H-[1,2,3]oxadiazolo[4,3-c][1,4]oxazin-8-ium-3-olate

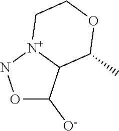

The title compound was prepared in a manner analogous to Intermediate 1, except using (2R,3S)-2-methylmorpholine-3-carboxylic acid instead of L-proline. MS (ESI): mass calcd. for C₆H₈N₂O₃, 156.1; m/z found, 157.0 [M+H]⁺. ¹H NMR (400 MHz, DMSO-d₆) δ 4.73 (q, J=6.50 Hz, 1H), 4.38-4.45 (m, 2H), 4.27 (dt, J=12.29, 3.49 Hz, 1H), 3.90-4.04 (m, 1H), 1.42 (d, J=6.50 Hz, 3H).

### Intermediate 17: 6,6-Dimethyl-6,7-dihydro-4H-[1,2,3]oxadiazolo[4,3-c][1,4]oxazin-8-ium-3-olate

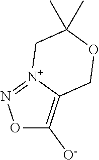

The title compound was prepared in a manner analogous to Intermediate 1, except using 6,6-dimethylmorpholine-3-carboxylic acid instead of L-proline. MS (ESI): mass calcd. for C₇H₁₀N₂O₃, 170.1; m/z found, 171.1 [M+H]⁺. ¹H NMR (500 MHz, DMSO-d₆) δ 4.64 (s, 2H), 4.31 (s, 2H), 1.31 (s, 6H).

### Intermediate 18: 5-((Benzyloxy)carbonyl)-4,5,6,7-tetrahydro-[1,2,3]oxadiazolo[3,4-a]pyrazin-8-ium-3-olate

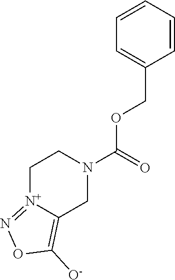 [SMILES: O=C(OCc1ccccc1)N1CC[n+]2noc([O-])c2C1]

The title compound was prepared in a manner analogous to Intermediate 1, except using 4-((benzyloxy)carbonyl)piperazine-2-carboxylic acid instead of morpholine-3-carboxylic acid in Step A. MS (ESI): mass calcd. for C₁₃H₁₃N₃O₄, 275.1; m/z found, 276.1 [M+H]⁺.

### Intermediate 19: 7-Chloro-1-((2-(trimethylsilyl)ethoxy)methyl)-1H-pyrazolo[4,3-b]pyridine

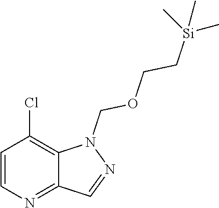 [SMILES: C[Si](C)(C)CCOCn1ncc2nccc(Cl)c21]

Sodium hydride in mineral oil (625 mg, 60% purity, 15.6 mmol) was added in portions to a 0° C. (ice/water) solution consisting of 7-chloro-1H-pyrazolo[4,3-b]pyridine (1.60 g, 10.4 mmol) and THF (15 mL). The resultant mixture was stirred at 0° C. for 1 hour and then treated with (2-(chloromethoxy)ethyl)trimethylsilane (3.30 mL, 18.6 mmol) added dropwise at 0° C. (ice/water). The resultant mixture was stirred for 12 hours. The reaction mixture was gradually warmed to room-temperature then quenched with water (20 mL) and extracted with ethyl acetate (50 mL×2). The combined organic extracts were dried over anhydrous Na₂SO₄, filtered, and concentrated to dryness under reduced pressure. The resulting residue was purified by FCC (SiO₂, eluent: petroleum ether:ethyl acetate=10:1 to 5:1) to afford the title compound (1.5 g, 46%) as a colourless oil. MS (ESI): mass calcd. for C₁₂H₁₈ClN₃OSi 283.1 m/z found 283.9 [M+H]⁺.

### Intermediate 20: 7-Bromo-2-methyl-2H-pyrazolo[4,3-b]pyridine

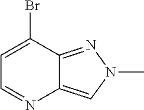

Sodium hydride in mineral oil (250 mg, 60% purity, 6.25 mmol) was added to a 0° C. solution consisting of 7-bromo-1H-pyrazolo[4,3-b]pyridine (600 mg, 3.03 mmol) and THF (12 mL). The resultant mixture was stirred at 0° C. for 0.5 hours before treating with Mel (3.16 g, 22.3 mmol) by dropwise. The mixture was stirred for 8 hours. The reaction mixture was gradually warmed to room-temperature then quenched with sat. NaHCO₃(10 mL) and extracted with ethyl acetate (100 mL×2). The combined organic extracts were dried over anhydrous Na₂SO₄, filtered, and concentrated to dryness under reduced pressure. The resulting residue was purified by FCC (SiO₂, eluent: petroleum ether:ethyl acetate=1:0 to 1:1) to afford the product (160 mg, 21%). MS (ESI): mass calcd. for C₇H₆BrN₃ 211.0; m/z found 212.1 [M+H]⁺. ¹H NMR (400 MHz, DMSO-d₆) δ 8.88 (s, 1H), 8.42 (d, J=4.6 Hz, 1H), 7.74 (d, J=4.8 Hz, 1H), 4.29-4.26 (m, 3H).

### Intermediate 21: 4-(4,4,5,5-Tetramethyl-1,3,2-dioxaborolan-2-yl)-1-((2-(trimethylsilyl)ethoxy)methyl)-1H-pyrrolo[2,3-b]pyridine

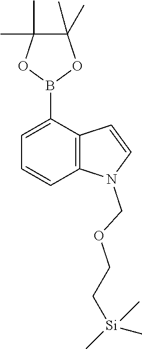

Step A: 4-Bromo-1-((2-(trimethylsilyl)ethoxy)methyl)-1H-pyrrolo[2,3-b]pyridine. To a solution of 4-bromo-1H-pyrrolo[2,3-b]pyridine (430 mg, 2.1 mmol) in DMF (4.3 mL) was added NaH (60% dispersion in mineral oil, 128 mg, 3.2 mmol). The reaction mixture was stirred for 20 minutes at room temperature than cooled to 0° C. followed by the slow addition of 2-(trimethylsilyl)ethoxymethyl chloride (0.42 mL, 2.4 mmol). The reaction mixture was then allowed to warm up to room temperature. After 16 hours, water was added and the aqueous phase was extracted twice with ethyl acetate. The combined organic layers were washed with a saturated aqueous solution of NaCl, dried over MgSO₄, filtered and evaporated. The resulting residue was purified by silica gel chromatography (0-100% EtOAc in hexanes) to afford the title compound (639 mg, 91% yield). MS (ESI): mass calcd. for C₁₃H₁₉BrN₂OSi, 326.1; m/z found, 327.0 [M+H]⁺.

Step B: 4-(4,4,5,5-Tetramethyl-1,3,2-dioxaborolan-2-yl)-1-((2-(trimethylsilyl)ethoxy)methyl)-1H-pyrrolo[2,3-b]pyridine. In a sealed vessel were combined 4-bromo-1-((2-(trimethylsilyl)ethoxy)methyl)-1H-pyrrolo[2,3-b]pyridine (639 mg, 1.95 mmol), 4,4,4′,4′,5,5,5′,5′-octamethyl-2,2′-bi(1,3,2-dioxaborolane) (595 mg, 2.3 mmol), potassium acetate (383 mg, 3.9 mmol), 1,4-dioxane (13 mL) and Pd(dppf)Cl₂ DCM (159 mg, 0.2 mmol). The reaction mixture was then degassed with nitrogen for 5 minutes, sealed and heated to 100° C. for 16 hours. The reaction mixture was cooled, filtered through a pad of Celite®, and filtrated was concentrated under reduced pressure. The resulting residue was purified by FCC (SiO₂, 0-100% EtOAc in hexanes) to afford the title compound (545 mg, 76% yield). MS (ESI): mass calcd. for C₁₉H₃₁BN₂O₃Si, 374.2; m/z found, 293.1 [(M-C₆H₁₀)+H]⁺. ¹H NMR (500 MHz, CDCl₃): δ 8.34 (d, J=4.6 Hz, 1H), 7.46 (d, J=4.6 Hz, 1H), 7.39 (d, J=3.5 Hz, 1H), 6.92 (d, J=3.6 Hz, 1H), 5.69 (s, 2H), 3.58-3.45 (m, 2H), 1.40 (s, 12H), 0.97-0.84 (m, 2H), −0.08 (s, 9H).

### Intermediate 22: (1-((2-(Trimethylsilyl)ethoxy)methyl)-1H-pyrazolo[3,4-b]pyridin-4-yl)boronic acid

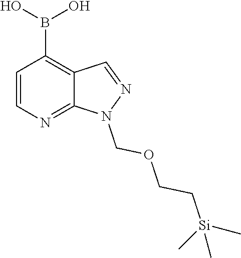 [SMILES: C[Si](C)(C)CCOCn1ncc2c(B(O)O)ccnc21]

The title compound was prepared in a manner analogous to Intermediate 21, Steps A-B, except using 4-bromo-1H-pyrazolo[3,4-b]pyridine instead of 4-bromo-1H-pyrrolo[2,3-b]pyridine in Step A. MS (ESI): mass calcd. for C₁₂H₂₀BN₃O₃Si, 293.1; m/z found, 294.1 [M+H]⁺.

### Intermediate 23: 4-(4,4,5,5-Tetramethyl-1,3,2-dioxaborolan-2-yl)-1-((2-(trimethylsilyl)ethoxy)methyl)-1H-pyrazolo[3,4-b]pyridine

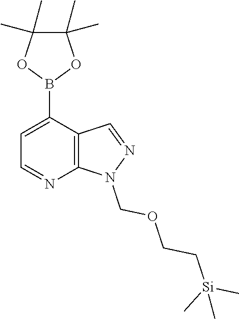

The title compound was prepared in a manner analogous to Intermediate 21, Steps A-B, except using 4-bromo-1H-pyrazolo[3,4-b]pyridine instead of 4-bromo-1H-pyrrolo[2,3-b]pyridine in Step A. MS (ESI): mass calcd. for C₁₈H₃₀BN₃O₃Si 375.2 m/z found 294.1 [M-C₆H₁₀)+H]⁺ and 376.2 [M+H]⁺. ¹H NMR (400 MHz, DMSO-d₆) δ 8.15 (s, 1H), 7.39 (s, 1H), 5.73 (s, 2H), 3.60-3.53 (m, 2H), 2.62 (s, 3H), 1.36 (s, 12H), 0.86-0.74 (m, 2H), 0.11 (s, 9H).

### Intermediate 24: 1-Benzyl-3,6-dimethyl-4-(4,4,5,5-tetramethyl-1,3,2-dioxaborolan-2-yl)-1H-pyrazolo[3,4-b]pyridine

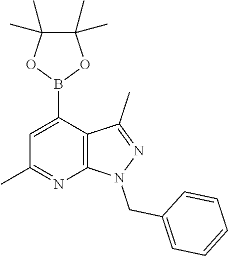 [SMILES: Cc1cc(B2OC(C)(C)C(C)(C)O2)c2c(C)nn(Cc3ccccc3)c2n1]

Step A: Methyl (E-3-((1-benzyl-3-methyl-1H-pyrazol-5-yl)imino)butanoate. A mixture of 1-benzyl-3-methyl-1H-pyrazol-5-amine (5.00 g, 26.7 mmol), methyl 3-oxobutanoate (5.59 g, 48.1 mmol) and TsOH (0.10 g, 0.53 mmol) in toluene (10V) was heated at 70° C. under N₂ for 14 hrs. The mixture was cooled to RT and filtered. The filter cake was washed with toluene (2V). The combined filtrate was concentrated to give a residue which was purified by chromatography on silica gel (PE-EA=20:1, 15:1, 10:1, 8:1) to afford the title compound as a yellow solid (6.3 g, 83%). MS (ESI): mass calcd. for C₁₆H₁₉N₃O₂, 285.1; m/z found, 286.2 [M+H]⁺. ¹HNMR (400 MHz, CDCl₃): δ 10.00 (s, 1H), 7.34-7.23 (m, 5H), 5.81 (s, 1H), 5.18 (s, 2H), 4.57 (s, 1H), 3.69 (s, 3H), 2.27 (s, 3H), 1.72 (s, 3H).

Step B: 1-Benzyl-3,6-dimethyl-1H-pyrazolo[3,4-b]pyridin-4-ol. Dowtherm A™ (48 mL, 8V) was heated up to 240° C. in a round bottom flask under N₂ and methyl (E)-3-((1-benzyl-3-methyl-1H-pyrazol-5-yl)imino)butanoate (6.0 g, 21.0 mmol) was added. The reaction mixture was stirred for 2 h, cooled to room temperature, and petroleum ether (48 mL, 8V) was added. The solid was collected by filtration and washed with petroleum ether twice to yield an off-white solid (5.0 g). Further purification by slurry with ethyl acetate and petroleum ether (V/V=1:5) afforded the title compound (4.5 g, 85%). MS (ESI): mass calcd. for C₁₅H₁₅N₃O, 253.1; m/z found, 254.2 [M+H]⁺.

Step C: 1-Benzyl-4-bromo-3,6-dimethyl-1H-pyrazolo[3,4-b]pyridine. To a mixture of 1-benzyl-3,6-dimethyl-1H-pyrazolo[3,4-b]pyridin-4-ol (4.5 g, 17.8 mmol) in toluene (45.0 ml, 10V) and DMF (13.5 ml, 3V) was added POBr₃ (6.1 g, 21.3 mmol) under N₂. The mixture was heated at 110° C. for 1 h and then cooled to RT. The reaction was quenched with cold water (225 mL, 50V) and then extracted with DCM (225 mL*2, 50V*2). The combined organic layers were concentrated to give a crude oil. Further purification by chromatography on silica gel (PE-EA=100:1 to 60:1 to 40:1 to 20:1) afforded the title compound (4.6 g, 82%). MS (ESI): mass calcd. for C₁₅H₁₄BrN₃, 315.0; m/z found, 316.0 [M+H]⁺. ¹H NMR (400 MHz, CDCl₃): δ 7.33-7.24 (m, 5H), 7.15 (s, 1H), 5.32 (s, 2H), 2.72 (s, 3H), 2.65 (s, 3H).

Step D: 1-Benzyl-3,6-dimethyl-4-(4,4,5,5-tetramethyl-1,3,2-dioxaborolan-2-yl)-1H-pyrazolo[3,4-b]pyridine. The title compound was made in a manner analogous to Intermediate 21, Step B except using 1-benzyl-4-bromo-3,6-dimethyl-1H-pyrazolo[3,4-b]pyridine instead of 4-bromo-1-((2-(trimethylsilyl)ethoxy)methyl)-1H-pyrrolo[2,3-b]pyridine (Intermediate 21, Step A). MS (ESI): mass calcd. for C₂₁H₂₆BN₃O₂ 363.2 m/z found 364.0 [M+H]⁺. ¹H NMR (400 MHz, CDCl₃): δ 7.37-7.28 (m, 4H), 7.27-7.20 (m, 1H), 6.88 (s, 1H), 5.79-5.69 (m, 1H), 5.66-5.59 (m, 1H), 2.71 (s, 3H), 1.91 (s, 3H), 1.26 (s, 12H).

### Intermediate 25: 1-Benzyl-4-bromo-5-fluoro-6-methyl-1H-pyrazolo[3,4-b]pyridine

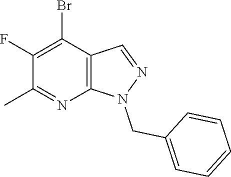 [SMILES: Cc1nc2c(cnn2Cc2ccccc2)c(Br)c1F]

Step A: Diethyl 2-(1-ethoxyethylidene)malonate. A solution of diethyl malonate (200.0 g, 1.25 mol) and ZnCl₂ (25.5 g, 0.187 mol) was heated to 140° C. under N₂. Triethyl orthoacetate (608.4 g, 3.75 mol) was added dropwise and the mixture was stirred at 140±5° C. The reaction mixture was cooled to room temperature and concentrated in vacuo. The residue was purified by chromatography on silica gel (PE:EA=50:1, 30:1 to 10:1) to yield the title compound (135.0 g, 47%). ¹H NMR (400 MHz, CDCl₃): δ 4.26 (q, J=7.2 Hz, 2H), 4.17 (q, J=7.1 Hz, 2H), 4.07 (q, J=7.0 Hz, 2H), 2.44 (s, 3H), 1.30 (td, J=7.1, 4.0 Hz, 6H), 1.25 (t, J=7.1 Hz, 3H).

Step B: Diethyl 2-(1-((1-benzyl-1H-pyrazol-5-yl)amino)ethylidene)malonate. A mixture of 1-benzyl-1H-pyrazol-5-amine (60.0 g, 0.35 mol) and diethyl 2-(1-ethoxyethylidene)malonate (122.0 g, 0.53 mol) was heated at 120° C. under N₂ for 12 hrs. After being cooled to room temperature, the reaction mixture was concentrated and purification by column chromatography (PE, PE:EA=30:1 to 10:1) afforded the title compound (89.0 g, 71%). MS (ESI): mass calcd. for C₁₉H₂₃N₃O₄, 357.2; m/z found, 358.2 [M+H]⁺. ¹H NMR (300 MHz, CDCl₃): δ 10.78 (s, 1H), 7.52 (s, 1H), 7.38-7.19 (m, 5H), 6.05 (s, 1H), 5.23 (s, 2H), 4.32-4.13 (m, 4H), 1.79 (s, 3H), 1.38-1.21 (m, 6H).

Step C: Ethyl 1-benzyl-4-hydroxy-6-methyl-1H-pyrazolo[3,4-b]pyridine-5-carboxylate. The title compound was prepared in a manner analogous to Intermediate 24, Step B, using diethyl 2-(1-((1-benzyl-1H-pyrazol-5-yl)amino)ethylidene)malonate instead of methyl (E)-3-((1-benzyl-3-methyl-1H-pyrazol-5-yl)imino)butanoate. MS (ESI): mass calcd. for C₁₇H₁₇N₃O₃, 311.1; m/z found, 312.1 [M+H]⁺. ¹H NMR (400 MHz, CDCl₃): δ 13.27 (s, 1H), 8.12 (s, 1H), 7.40-7.23 (m, 5H), 5.65 (s, 2H), 4.51 (q, J=7.1 Hz, 2H), 2.89 (s, 3H), 1.50 (t, J=7.1 Hz, 3H).

Step D: 1-Benzyl-4-hydroxy-6-methyl-1H-pyrazolo[3,4-b]pyridine-5-carboxylic acid. To a solution of ethyl 1-benzyl-4-hydroxy-6-methyl-1H-pyrazolo[3,4-b]pyridine-5-carboxylate (58.0 g, 0.186 mol) in EtOH (290 mL, 5V) was added NaOH (22.4 g, 0.56 mol) dissolved in H₂O (116 mL, 2V) and the mixture was heated to reflux (78° C.). After 3 hrs additional NaOH (22.4 g, 0.56 mol) dissolved in H₂O (29 mL, 0.5V) was added into the reaction mixture. After an additional 3 hrs, the reaction was cooled to RT and concentrated to remove EtOH. H₂O (870 mL, 15V) was added and the pH was adjusted to pH=2 with conc. HCl. The solid was collected by filtration and dried in vacuo to afford the title compound (50.0 g, 95%). MS (ESI): mass calcd. for C₁₅H₁₃N₃O₃, 283.1; m/z found, 284.1 [M+H]⁺.

Step E: 1-Benzyl-4-bromo-6-methyl-1H-pyrazolo[3,4-b]pyridine-5-carboxylic acid. The title compound was prepared in a manner analogous to Intermediate 24, Step C using 1-benzyl-4-hydroxy-6-methyl-1H-pyrazolo[3,4-b]pyridine-5-carboxylic acid instead of 1-benzyl-3,6-dimethyl-1H-pyrazolo[3,4-b]pyridin-4-ol. MS (ESI): mass calcd. for C₁₅H₁₂BrN₃O₂, 345.0 m/z found, 346.0 [M+H]⁺.

Step F: tert-Butyl (1-benzyl-4-bromo-6-methyl-1H-pyrazolo[3,4-b]pyridin-5-yl)carbamate. A mixture of 1-benzyl-4-bromo-6-methyl-1H-pyrazolo[3,4-b]pyridine-5-carboxylic acid (33.0 g, 95.4 mmol), DPPA (39.3 g, 143.2 mmol), TEA (19.3 g, 190.8 mmol), t-BuOH (21.2 g, 286.2 mmol) and toluene (330 mL, 10V) was heated to 80-90° C. for 4 hrs. The reaction was cooled to room temperature and water (330 mL, 10V) was added. The aqueous mixture was extracted with ethyl acetate (330 mL, 10V) and the organic layers were concentrated in vacuo to give 15.8 g crude oil with 94% purity by LCMS which was used directly for next step. MS (ESI): mass calcd. for C₁₉H₂₁BrN₄O₂, 416.1 m/z found, 417.1 [M+H]⁺.

Step G: 1-Benzyl-4-bromo-6-methyl-1H-pyrazolo[3,4-b]pyridin-5-amine. To tert-butyl (1-benzyl-4-bromo-6-methyl-1H-pyrazolo[3,4-b]pyridin-5-yl)carbamate (15.8 g, crude) (obtained from Step F) in dichloromethane (64 mL, 4V) was added TFA (64 ml, 4V) at 0° C. The mixture was warmed to room temperature and stirred for 30 minutes. The solvent was removed under reduced pressure and the residue was basified with aqueous NaHCO₃(50 mL), extracted with dichloromethane (50 mL×2). The organic layers were combined and concentrated. The residue was purified by chromatography to give 10.5 g 1-benzyl-4-bromo-6-methyl-1H-pyrazolo[3,4-b]pyridin-5-amine with 97.1% purity by LCMS and 34.7% yield for 2 steps. MS (ESI): mass calcd. for C₁₄H₁₃BrN₄, 316.0 m/z found, 317.0 [M+H]⁺.

Step H: 1-Benzyl-4-bromo-5-fluoro-6-methyl-1H-pyrazolo[3,4-b]pyridine. To a mixture of 1-benzyl-4-bromo-6-methyl-1H-pyrazolo[3,4-b]pyridin-5-amine (9.0 g, 28.4 mmol, 1.0 eq) and ACN (45 mL, 5V) was added HBF₄·Et₂O (5.5 g, 34.1 mmol, 1.2 eq). The mixture was stirred to give a clear solution. Then isopentyl nitrite (3.99 g, 34.0 mmol) was added dropwise at 0° C. The solvent was removed under reduced pressure to give crude 1-benzyl-4-bromo-6-methyl-1H-pyrazolo[3,4-b]pyridine-5-diazonium tetrafluoroborate, which was slurried in heptane (90 mL, 10V) to afford 1-benzyl-4-bromo-6-methyl-1H-pyrazolo[3,4-b]pyridine-5-diazonium tetrafluoroborate as a solid, 11.7 g with 99% yield. To a reactor was added [BMIM]BF₄ (10 mL, 50V) which was heated to 200° C. 1-Benzyl-4-bromo-6-methyl-1H-pyrazolo[3,4-b]pyridine-5-diazonium tetrafluoroborate (2.0 g, 1.20 mmol) was added to the reactor quickly and the resulting mixture was stirred for 5 min. Then the reaction was cooled to 30° C. quickly. Multiple batches were combined (12.5 g) for workup. Water (30 mL, 15V) was added and extracted with ethyl acetate (20 mL×2, 10V×2). The organic phase was removed under reduced pressure to give crude 1-benzyl-4-bromo-5-fluoro-6-methyl-1H-pyrazolo[3,4-b]pyridine, which was further purified by chromatography to give the title compound (720 mg, 7.5%). MS (ESI): mass calcd. for C₁₄H₁₁BrFN₃, 319.0 m/z found, 321.0 [M+H]⁺. ¹H NMR (400 MHz, DMSO-d₆) δ 7.96 (s, 1H), 7.36-7.26 (m, 5H), 5.68 (s, 2H), 2.71-2.68 (m, 3H).

### Intermediate 26: 6-Methyl-4-(4,4,5,5-tetramethyl-1,3,2-dioxaborolan-2-yl)-1-((2-(trimethylsilyl)ethoxy)methyl)-1H-pyrazolo[3,4-b]pyridine

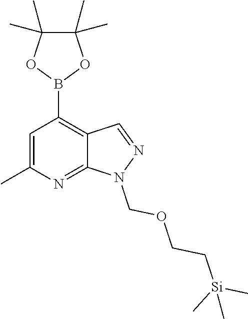

The title compound was prepared in a manner analogous to Intermediate 21, Steps A-B, except using 4-bromo-6-methyl-1H-pyrazolo[3,4-b]pyridine instead of 4-bromo-1H-pyrrolo[2,3-b]pyridine in Step A. MS (ESI): mass calcd. for C₁₉H₃₂BN₃O₃Si, 389.2; mass calcd. for C₁₃H₂₂BN₃O₃Si (hydrolyzed BPin ester), 307.2; m/z found, 308.2 [M+H]⁺.

¹H NMR (500 MHz, CDCl₃): δ ppm 8.3 (s, 1H) 7.4 (s, 1H) 5.9 (s, 2H) 3.6-3.7 (m, 2H) 2.7-2.7 (m, 3H) 1.3-1.4 (m, 12H) 0.9-1.0 (m, 2H) −0.1-0.0 (m, 9H).

### Intermediate 27: 2-(4,4,5,5-Tetramethyl-1,3,2-dioxaborolan-2-yl)-6,7-dihydro-4H-pyrazolo[5,1-c][1,4]oxazine

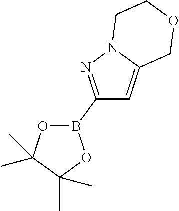 [SMILES: CC1(C)OB(c2cc3n(n2)CCOC3)OC1(C)C]

A solution of 6,7-dihydro-4H-[1,2,3]oxadiazolo[4,3-c][1,4]oxazin-8-ium-3-olate (Intermediate 2, 118 mg, 0.83 mmol) and 2-ethynyl-4,4,5,5-tetramethyl-1,3,2-dioxaborolane (252 mg, 1.66 mmol) in xylenes (0.8 mL) was heated to 150° C. for 16 h. The reaction mixture was cooled to RT then concentrated. Purification by chromatography FCC (silica gel, 0-100% EtOAc/hexanes) afforded 44 mg (21%) of the title compound. MS (ESI): mass calcd. for C₁₂H₁₉BN₂O₃, 250.1; m/z found, 251.1 [M+H]⁺. ¹H NMR (400 MHz, CDCl₃): δ 6.43 (s, 1H), 4.87 (s, 2H), 4.33 (t, J=5.19 Hz, 2H), 4.15-4.06 (m, 2H), 1.38-1.35 (m, 12H).

### Intermediate 28: 2-(Difluoromethyl)-4-(4,4,5,5-tetramethyl-1,3,2-dioxaborolan-2-yl)pyridine

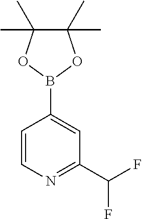 [SMILES: CC1(C)OB(c2ccnc(C(F)F)c2)OC1(C)C]

To a vial containing 4-bromo-2-(difluoromethyl)pyridine (62 mg, 0.3 mmol), bis(pinacolato)diboron (91 mg, 0.36 mmol), potassium acetate (59 mg, 0.6 mmol) and [1,1′-bis(diphenylphosphino)ferrocene]dichloropalladium(II) (24 mg, 0.03 mmol) under N₂ was added 1,4 dioxane. The vial was capped and the reaction heated to 90° C. for 3 hours. The mixture was allowed to cool to RT then diluted with EtOAc and filtered through a pad of Celite®. The filtrate was condensed to afford the title compound, which was used without further purification. MS (ESI): mass calcd. for C₆H₆BF₂NO₂, 173.0; m/z found, 174.1 [M+H]⁺.

### Intermediate 29: 7-(4,4,5,5-Tetramethyl-1,3,2-dioxaborolan-2-yl)thieno[3,2-b]pyridine dwu23_2379

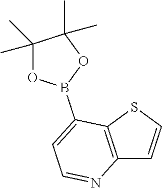 [SMILES: CC1(C)OB(c2ccnc3ccsc23)OC1(C)C]

The title compound was prepared in a manner analogous to Intermediate 28, except using 7-bromothieno[3,2-b]pyridine instead of 4-bromo-2-(difluoromethyl)pyridine. MS (ESI): mass calcd. for C₁₃H₁₆BNO₂S, 261.1; mass calcd. for C₇H₆BNO₂S (hydrolyzed BPin ester), 179.0; m/z found, 180.1 [M+H]⁺.

### Intermediate 30: 2-Methoxy-8-(4,4,5,5-tetramethyl-1,3,2-dioxaborolan-2-yl)-1,5-naphthyridine

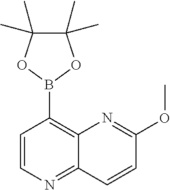

The title compound was prepared in a manner analogous to Intermediate 28, except using 8-bromo-2-methoxy-1,5-naphthyridine instead of 4-bromo-2-(difluoromethyl)pyridine. MS (ESI): mass calcd. for C₁₅H₁₉BN₂O₃, 286.1; mass calcd. for C₉H₉BN₂O₃ (hydrolyzed BPin ester), 204.1; m/z found, 205.2 [M+H]⁺.

### Intermediate 31: 1-Ethyl-5-(4,4,5,5-tetramethyl-1,3,2-dioxaborolan-2-yl)-1H-pyrazolo[3,4-b]pyridine

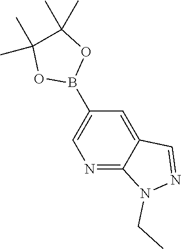 [SMILES: CCn1ncc2cc(B3OC(C)(C)C(C)(C)O3)cnc21]

The title compound was prepared in a manner analogous to Intermediate 28, except using 5-bromo-1-ethyl-1H-pyrazolo[3,4-b]pyridine instead of 4-bromo-2-(difluoromethyl)pyridine and DME instead of 1,4 dioxane. MS (ESI): mass calcd. for C₁₄H₂₀BN₃O₂, 273.1; m/z found, 274.3 [M+H]⁺.

### Intermediate 32: 2-(Difluoromethyl)-1-(phenylsulfonyl)-4-(4,4,5,5-tetramethyl-1,3,2-dioxaborolan-2-yl)-1H-pyrrolo[2,3-b]pyridine

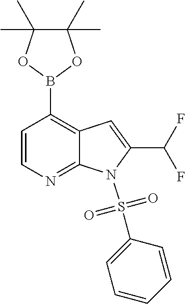 [SMILES: CC1(C)OB(c2ccnc3c2cc(C(F)F)n3S(=O)(=O)c2ccccc2)OC1(C)C]

Step A. 4-Bromo-2-(difluoromethyl)-1-(phenylsulfonyl)-1H-pyrrolo[2,3-b]pyridine. To a 0° C. solution of triethylamine trihydrofluoride (1.85 mL, 11.1 mmol) in dichloromethane (41 mL) was successively added XtalFluor-E® (1.88 g, 8.2 mmol) and 4-bromo-1-phenylsulfonyl-7-azaindole-2-carboxaldehyde (1.5 g, 4.1 mmol). After 30 minutes, the reaction was allowed to warm to room temperature. The reaction was quenched with saturated NaHCO₃ (aq) and extracted with dichloromethane (2×). The combined organics were dried over Na₂SO₄, filtered, and condensed. Purification by chromatography (silica gel, 0-100% EtOAc/hexanes) afforded 765 mg (48%) of the title compound. MS (ESI): mass calcd. for C₁₄H₉BrF₂N₂O₂S, 386.0; m/z found, 386.9 [M+H]⁺. ¹H NMR (400 MHz, CDCl₃): δ 8.31 (d, J=5.25 Hz, 1H) 8.26-8.29 (m, 2H) 7.60-7.65 (m, 1H) 7.49-7.55 (m, 2H) 7.43 (d, J=5.13 Hz, 1H) 7.44 (t, J=54.47 Hz, 1H) 7.06 (d, J=0.75 Hz, 1H).

Step B. 2-(Difluoromethyl)-1-(phenylsulfonyl)-4-(4,4,5,5-tetramethyl-1,3,2-dioxaborolan-2-yl)-1H-pyrrolo[2,3-b]pyridine. In a pressure vessel was dissolved 4-bromo-2-(difluoromethyl)-1-(phenylsulfonyl)-1H-pyrrolo[2,3-b]pyridine (50 mg, 0.13 mmol), potassium acetate (38 mg, 0.39 mmol), bis(pinacolato)diboron (49 mg, 0.19 mmol), and [1,1′-bis(diphenylphosphino)ferrocene]dichloropalladium(II) in 1,4-dioxane (0.65 mL). The resulting mixture was degassed with N₂ and heated for 2 h at 80° C. The reaction was cooled to room temperature and partitioned between ethyl acetate and H₂O. The layers were separated and the aqueous was extracted with ethyl acetate (2×5 mL). The organic layers were combined and washed with brine (5 mL), dried (Na₂SO₄), filtered through Celite®, and condensed. Used without further purification. MS (ESI): mass calcd. for C₁₄H₁₁BF₂N₂O₄S (hydrolyzed BPin ester), 352.1; m/z found, 353.1 [M+H]⁺. ¹H NMR (400 MHz, DMSO-d₆) δ 8.49 (d, J=4.63 Hz, 1H) 8.12-8.19 (m, 2H) 7.92 (s, 1H) 7.57-7.78 (m, 3H) 7.55 (d, J=4.63 Hz, 1H) 7.28 (s, 1H) 1.33 (s, 12H).

### Intermediate 33: Ethyl 3-(4-fluorophenyl)-1H-pyrazole-5-carboxylate

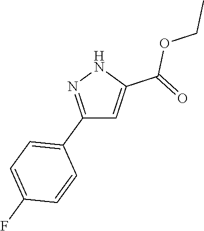 [SMILES: CCOC(=O)c1cc(-c2ccc(F)cc2)n[nH]1]

Ethyl 2-diazoacetate (3.9 mL, 37 mmol), 1-ethynyl-4-fluorobenzene (3.0 g, 25 mmol), and toluene (15 mL) were added to a 20 mL microwave tube. The resultant mixture was heated at 105° C. via microwave irradiation for 2 hours. The reaction mixture was cooled to room-temperature. The mixture was combined with additional batches and concentrated to dryness under reduced pressure. The residue was triturated with petroleum ether:ethyl acetate (30:1, 20 mL) and the resultant suspension isolated via filtration. The filter cake was washed with petroleum ether (10 mL) before drying under reduced pressure to afford the title compound (4.0 g) as a white solid. The combined filtrate was concentrated to dryness under reduced pressure. The resulting residue was purified by FCC (SiO₂, eluent: petroleum ether:ethyl acetate=10:1 to 4:1) to afford the title compound (0.8 g) as a white solid. LC-MS (ESI): mass calcd. for C₁₂H₁₁FN₂O₂ 234.08 m/z found 235.1 [M+H]⁺.

### Intermediate 34: Ethyl 3-(5-fluoropyridin-2-yl)-1H-pyrazole-5-carboxylate

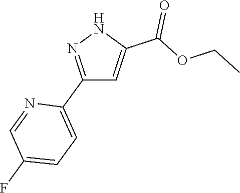 [SMILES: CCOC(=O)c1cc(-c2ccc(F)cn2)n[nH]1]

Method A:

Step A. Ethyl 4-(5-fluoropyridin-2-yl)-2,4-dioxobutanoate. 1-(5-Fluoropyridin-2-yl)ethan-1-one (4.03 g, 28.99 mmol), diethyl oxalate (5.91 mL, 1.08 g/mL, 43.48 mmol) and sodium tert-butoxide (5.01 g, 52.18 mmol) were dissolved in EtOH, and stirred at RT for 15 h. The resulting mixture was diluted with HCl (1M, 25 mL), then water (200 mL) to precipitate the product as a white solid. The solid was filtered off and analyzed by LCMS then carried forward without further purification. MS (ESI): mass calcd. for C₁₁H₁₀FNO₄, 239.20; m/z found, 240.1 [M+H]⁺.

Step B. Ethyl 3-(5-fluoropyridin-2-yl)-1H-pyrazole-5-carboxylate. Ethyl 4-(5-fluoropyridin-2-yl)-2,4-dioxobutanoate (6 g, 25.08 mmol) was dissolved in AcOH (26.73 mL, 1.049 g/mL, 466.97 mmol) and hydrazine (3.97 mL, 1.021 g/mL, 123.87 mmol) was added dropwise over 5 min after which the reaction was allowed to stir at RT for 15 h. The reaction was then diluted with 2M HCl (10 mL) and deionized water (200 mL) which precipitated the product, ethyl 3-(5-fluoropyridin-2-yl)-1H-pyrazole-5-carboxylate, which was filtered off as a white solid and lyophilized for 24 h to yield 2.3 g white powder. MS (ESI): mass calcd. for C₁₁H₁₀FN₃O₂, 235.2; m/z found, 236.1 [M+H]⁺.

Method B:

Ethyl 2-diazoacetate (6.6 mL, 62 mmol), 2-ethynyl-5-fluoropyridine (5.0 g, 41 mmol), and toluene (50 mL) were added to a 100 mL sealed tube. The resultant mixture was heated at 90° C. for 16 hours. The reaction mixture was cooled to room-temperature and concentrated to dryness under reduced pressure. The residue was combined with additional batches, triturated with petroleum ether:ethyl acetate=15:1 (100 mL), and the resultant suspension isolated via filtration. The filter cake was washed with petroleum ether:ethyl acetate=15:1 (100 mL) before drying under reduced pressure to afford the title compound (13 g) as a yellow solid. LC-MS (ESI): mass calcd. for C₁₁H₁₀FN₃O₂ 235.08 m/z found 236.1 [M+H]⁺.

### Intermediate 35: 2-(5-(((tert-Butyldimethylsilyl)oxy)methyl)-1H-pyrazol-3-yl)-5-fluoropyridine

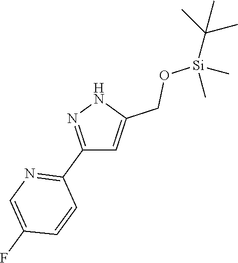 [SMILES: CC(C)(C)[Si](C)(C)OCc1cc(-c2ccc(F)cn2)n[nH]1]

Method A

Step A. (3-(5-Fluoropyridin-2-yl)-1H-pyrazol-5-yl) methanol. LiAlH₄ (4.72 g, 124 mmol) was added to a 0° C. (ice/water) solution consisting of ethyl 3-(5-fluoropyridin-2-yl)-1H-pyrazole-5-carboxylate (Intermediate 34, 11.7 g, 49.7 mmol) and THF (200 mL). The resultant mixture was stirred for 2 hours. The reaction mixture was gradually warmed to room-temperature then quenched with H₂O (5 mL) and aq. NaOH (15 wt %, 5 mL) slowly. The reaction mixture was stirred at room-temperature for 0.5 hours when an additional portion of H₂O (15 mL) was added. The resultant mixture was stirred at room-temperature for another 0.5 hours and then dried over anhydrous MgSO₄. The suspension was filtered through a pad of Celite® and the pad washed with ethyl acetate (200 mL). The filtrate was concentrated to dryness under reduced pressure to afford the title compound (8.8 g), which was used in the next step without further purification. LC-MS (ESI): mass calcd. for C₉H₈FN₃O 193.07 m/z found 194.1 [M+H]⁺.

Step B. 2-(5-(((tert-Butyldimethylsilyl)oxy)methyl)-1H-pyrazol-3-yl)-5-fluoropyridine. TBSCl (10.3 g, 68.3 mmol) was added to a solution consisting of (3-(5-fluoropyridin-2-yl)-1H-pyrazol-5-yl)methanol (8.5 g), 1H-imidazole (9.3 g, 137 mmol), dichloromethane (80 mL), and DMF (4 mL). The resultant mixture was stirred at room-temperature for 30 minutes. The suspension was filtered through a pad of Celite® and the pad washed with ethyl acetate (100 mL). The filtrate was concentrated to dryness under reduced pressure. The resulting residue was purified by FCC (SiO₂, eluent: petroleum ether:ethyl acetate=1:0 to 3:1) to afford the title compound (7.6 g, 54%) as a yellow solid. LC-MS (ESI): mass calcd. for C₁₅H₂₂FN₃OSi 307.15 m/z found 308.2 [M+H]⁺.

Or

Method B

TBSCl (3.0 g, 20 mmol) was added to a solution consisting of (3-(5-fluoropyridin-2-yl)-1H-pyrazol-5-yl)methanol (Intermediate 35, product from Step A, 2.6 g), 1H-imidazole (2.75 g, 40.4 mmol), CH₂Cl₂ (40 mL), and DMF (5 mL). The resultant mixture was stirred at room-temperature for 30 minutes. The suspension was filtered through a pad of Celite® and the pad washed with ethyl acetate (80 mL). The combined organic were concentrated to dryness under reduced pressure. The resulting residue was purified by FCC (SiO₂, eluent: petroleum ether:ethyl acetate=1:0 to 3:1) to afford the title compound (1.4 g) as a white solid. LCMS (ESI): mass calcd. for C₁₅H₂₂FN₃OSi 307.15 m/z, found 308.1 [M+1]⁺. Total run time was 9.5 minutes. ¹H NMR (400 MHz, DMSO-d₆) δ 13.33-12.93 (m, 1H), 8.65-8.51 (m, 1H), 8.04-7.89 (m, 1H), 7.87-7.68 (m, 1H), 6.81-6.64 (m, 1H), 4.77-4.61 (m, 2H), 0.88 (s, 9H), 0.07 (s, 6H).

### Intermediate 36: di-D-(3-(5-Fluoropyridin-2-yl)-1H-pyrazol-5-yl)methanol

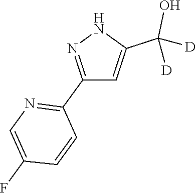 [SMILES: [2H]C([2H])(O)c1cc(-c2ccc(F)cn2)n[nH]1]

LiAlD₄ (390 mg, 9.29 mmol) was added to a solution consisting of ethyl 3-(5-fluoropyridin-2-yl)-1H-pyrazole-5-carboxylate (Intermediate 34, 1.0 g, 4.3 mmol) and THF (30 mL) under N₂ at room-temperature. The resultant mixture was stirred at room-temperature for 2 hours under N₂ before diluting with THF (20 mL). The mixture was quenched with H₂O (0.4 mL) slowly and then with 15% NaOH_{(aq)} (0.4 mL) slowly. The resultant mixture was stirred at room-temperature for 0.5 hours before diluting with H₂O (1.2 mL). The resultant mixture was stirred at room-temperature for another 0.5 hours before treating with anhydrous MgSO₄. The suspension was filtered through a pad of Celite® and the pad washed with ethyl acetate (30 mL). The filtrate was concentrated to dryness under reduced pressure to afford the title compound (1.2 g), which was used in the next step without further purification. LC-MS (ESI): mass calcd. for C₉H₆D₂FN₃O 195.08 m/z found 196.1 [M+H]⁺.

### Intermediate 37: 3-Bromo-2-(5-fluoropyridin-2-yl)-6,7-dihydro-4H-pyrazolo[5,1-c][1,4]oxazine

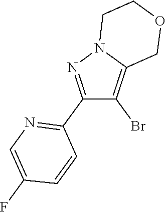 [SMILES: Fc1ccc(-c2nn3c(c2Br)COCC3)nc1]

Step A: 2-(5-Fluoropyridin-2-yl)-6,7-dihydro-4H-pyrazolo[5,1-c][1,4]oxazine and 3-(5-Fluoropyridin-2-yl)-6,7-dihydro-4H-pyrazolo[5,1-c][1,4]oxazine. To 6,7-dihydro-4H-[1,2,3]oxadiazolo[4,3-c][1,4]oxazin-8-ium-3-olate (Intermediate 2, 248 mg, 1.7 mmol) in xylenes (1.75 mL) was added 2-ethynyl-5-fluoropyridine (445 mg, 3.5 mmol). The reaction mixture was stirred at 150° C. for 16 hours. The crude material was directly purified via silica gel chromatography (0-100% EtOAc in hexanes) to give a mixture of the title compounds (87% of the desired cyclization product 2-(5-fluoropyridin-2-yl)-6,7-dihydro-4H-pyrazolo[5,1-c][1,4]oxazine, 312 mg, 82%). MS (ESI): mass calcd. for C₁₁H₁₀FN₃O, 219.1; m/z found, 220.1 [M+H]⁺. ¹H NMR (400 MHz, CDCl₃): δ 8.46 (d, J=2.9 Hz, 1H), 7.91 (ddd, J=8.8, 4.5, 0.6 Hz, 1H), 7.43 (ddd, J=8.8, 8.2, 2.9 Hz, 1H), 6.59-6.56 (m, 1H), 4.89 (d, J=0.8 Hz, 2H), 4.29-4.24 (m, 2H), 4.17-4.12 (m, 2H). Reporting only the major desired cyclization product.

Step B: 3-Bromo-2-(5-fluoropyridin-2-yl)-6,7-dihydro-4H-pyrazolo[5,1-c][1,4]oxazine. To a solution of 2-(5-fluoropyridin-2-yl)-6,7-dihydro-4H-pyrazolo[5,1-c][1,4]oxazine and 3-(5-fluoropyridin-2-yl)-6,7-dihydro-4H-pyrazolo[5,1-c][1,4]oxazine (250 mg, 1.1 mmol) in DMF (4.6 mL) was added N-bromosuccinimide (223 mg, 1.3 mmol). The reaction mixture was stirred at room temperature for 3 hours and then was diluted with ethyl acetate. The organic phase was washed twice with water, once with a saturated aqueous solution of NaCl, separated, dried over MgSO₄ and evaporated. The resulting residue was purified by silica gel chromatography (0-100% EtOAc in hexanes) gave the title compound (275 mg, 81% yield). MS (ESI): mass calcd. for C₁₁H₉BrFN₃O, 297.0; m/z found, 298.0 [M+H]⁺. ¹H NMR (400 MHz, CDCl₃): δ 8.57 (d, J=2.9 Hz, 1H), 7.98 (ddd, J=8.7, 4.4, 0.6 Hz, 1H), 7.46 (ddd, J=8.8, 8.1, 3.0 Hz, 1H), 4.79 (s, 2H), 4.26-4.21 (m, 2H), 4.16-4.10 (m, 2H).

### Intermediate 38: 3-Bromo-2-(4-fluorophenyl)-5,6-dihydro-4H-pyrrolo[1,2-b]pyrazole

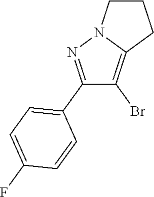 [SMILES: Fc1ccc(-c2nn3c(c2Br)CCC3)cc1]

The title compound was made in a manner analogous to Intermediate 37, Step A-B except using 1-ethynyl-4-fluorobenzene instead of 2-ethynyl-5-fluoropyridine, 5,6-dihydro-4H-pyrrolo[1,2-c][1,2,3]oxadiazol-7-ium-3-olate (Intermediate 1) instead of 6,7-dihydro-4H-[1,2,3]oxadiazolo[4,3-c][1,4]oxazin-8-ium-3-olate (Intermediate 2). MS (ESI): mass calcd. for C₁₂H₁₀BrFN₂, 280.0; m/z found, 281.1 [M+H]⁺.

### Intermediate 39: 3-Bromo-2-(5-fluoropyridin-2-yl)-5,6-dihydro-4H-pyrrolo[1,2-b]pyrazole

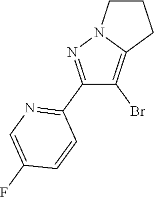 [SMILES: Fc1ccc(-c2nn3c(c2Br)CCC3)nc1]

The title compound was prepared in a manner analogous to Intermediate 37, Steps A-B, except using 5,6-dihydro-4H-pyrrolo[1,2-c][1,2,3]oxadiazol-7-ium-3-olate (Intermediate 1) instead of 6,6-dimethyl-6,7-dihydro-4H-[1,2,3]oxadiazolo[4,3-c][1,4]oxazin-8-ium-3-olate (Intermediate 17) in Step A. MS (ESI): mass calcd. for C₁₁H₉BrFN₃, 281.0; m/z found, 282.0 [M+H]⁺. ¹H NMR (500 MHz, DMSO-d₆) δ 8.61 (d, J=3.00 Hz, 1H), 7.88 (dd, J=8.76, 4.50 Hz, 1H), 7.78 (td, J=8.82, 3.00 Hz, 1H), 4.20 (t, J=7.32 Hz, 2H), 2.90-2.81 (m, 2H), 2.62-2.54 (m, 2H).

### Intermediate 40: 3-Bromo-5-fluoro-2-(5-fluoropyridin-2-yl)-5,6-dihydro-4H-pyrrolo[1,2-b]pyrazole

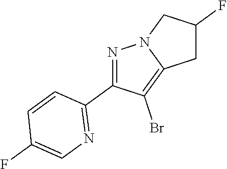 [SMILES: Fc1ccc(-c2nn3c(c2Br)CC(F)C3)nc1]

The title compound was made in a manner analogous to Intermediate 37, Step A-B except using (R)-5-fluoro-5,6-dihydro-4H-pyrrolo[1,2-c][1,2,3]oxadiazol-7-ium-3-olate (Intermediate 138) instead of 6,7-dihydro-4H-[1,2,3]oxadiazolo[4,3-c][1,4]oxazin-8-ium-3-olate (Intermediate 2) in Step A. MS (ESI): mass calcd. for C₁₁H₈BrF₂N₃, 299.0; found 299.9 [M+H]⁺. ¹H NMR (500 MHz, DMSO-d₆) δ 8.61 (d, J=3.0 Hz, 1H), 7.88 (dd, J=8.8, 4.5 Hz, 1H), 7.77 (td, J=8.8, 2.9 Hz, 1H), 5.95-5.80 (m, 1H), 4.59-4.47 (m, 1H), 4.42 (dd, J=25.7, 13.4 Hz, 1H), 3.43-3.30 (m, 1H), 3.06 (dd, J=26.1, 17.6 Hz, 1H).

### Intermediate 41: (S)-3-Bromo-5-fluoro-2-(4-fluorophenyl)-5,6-dihydro-4H-pyrrolo[1,2-b]pyrazole

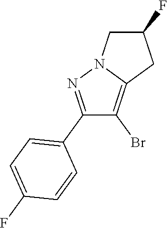 [SMILES: Fc1ccc(-c2nn3c(c2Br)C[C@H](F)C3)cc1]

The title compound was prepared in a manner analogous to Intermediate 37, except using (S)-5-fluoro-5,6-dihydro-4H-pyrrolo[1,2-c][1,2,3]oxadiazol-7-ium-3-olate (Intermediate 5) instead of 6,7-dihydro-4H-[1,2,3]oxadiazolo[4,3-c][1,4]oxazin-8-ium-3-olate (Intermediate 2) and 1-ethynyl-4-fluorobenzene instead of 2-ethynyl-5-fluoropyridine. MS (ESI): mass calcd. for C₁₂H₉BrF₂N₂, 298.0; m/z found, 299.0 [M+H]⁺.

### Intermediate 42: (S)-3-Bromo-2-(4-fluorophenyl)-6-methyl-5,6-dihydro-4H-pyrrolo[1,2-b]pyrazole

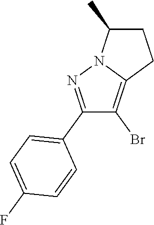 [SMILES: C[C@H]1CCc2c(Br)c(-c3ccc(F)cc3)nn21]

The title compound was prepared in a manner analogous to Intermediate 37 Steps A-B except using (S)-6-methyl-5,6-dihydro-4H-pyrrolo[1,2-c][1,2,3]oxadiazol-7-ium-3-olate (Intermediate 3) instead of 6,7-dihydro-4H-[1,2,3]oxadiazolo[4,3-c][1,4]oxazin-8-ium-3-olate (Intermediate 2), 1-ethynyl-4-fluorobenzene instead of 2-ethynyl-5-fluoropyridine, diphenyl ether instead of xylenes and also microwave irradiation at 240° C. for 2 h rather than conventional heating. DCM was used instead of DMF in Step B. MS (ESI): mass calcd. for C₁₃H₁₂BrFN₂ 294.0 m/z found 294.9 [M+H]⁺.

### Intermediate 43: (R)-3-Bromo-2-(4-fluorophenyl)-6-methyl-5,6-dihydro-4H-pyrrolo[1,2-b]pyrazole

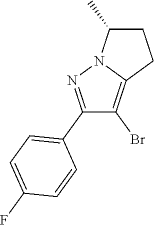 [SMILES: C[C@@H]1CCc2c(Br)c(-c3ccc(F)cc3)nn21]

The title compound was prepared in a manner analogous to Intermediate 37 Steps A-B except using (R)-6-methyl-5,6-dihydro-4H-pyrrolo[1,2-c][1,2,3]oxadiazol-7-ium-3-olate (Intermediate 4) instead of 6,7-dihydro-4H-[1,2,3]oxadiazolo[4,3-c][1,4]oxazin-8-ium-3-olate (Intermediate 2), 1-ethynyl-4-fluorobenzene instead of 2-ethynyl-5-fluoropyridine, diphenyl ether instead of xylenes and also microwave irradiation at 240° C. for 1 h rather than conventional heating. DCM was used instead of DMF in Step B. MS (ESI): mass calcd. for C₁₃H₁₂BrFN₂ 294.0 m/z found 294.9 [M+H]⁺.

### Intermediate 44: (S)-3-Bromo-2-(5-fluoropyridin-2-yl)-6-methyl-5,6-dihydro-4H-pyrrolo[1,2-b]pyrazole

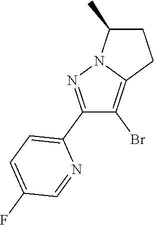 [SMILES: C[C@H]1CCc2c(Br)c(-c3ccc(F)cn3)nn21]

The title compound was prepared in a manner analogous to Intermediate 37 Steps A-B, except using (S)-6-methyl-5,6-dihydro-4H-pyrrolo[1,2-c][1,2,3]oxadiazol-7-ium-3-olate (Intermediate 3) instead of 6,7-dihydro-4H-[1,2,3]oxadiazolo[4,3-c][1,4]oxazin-8-ium-3-olate (Intermediate 2), diphenyl ether instead of xylenes and also microwave irradiation at 240° C. for 1 h rather than conventional heating. DCM was used instead of DMF in Step B. MS (ESI): mass calcd. for C₁₂H₁₁BrFN₃ 295.0 m/z found 295.9[M+H]⁺.

### Intermediate 45: (R)-3-Bromo-2-(5-fluoropyridin-2-yl)-6-methyl-5,6-dihydro-4H-pyrrolo[1,2-b]pyrazole

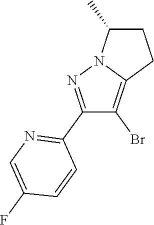 [SMILES: C[C@@H]1CCc2c(Br)c(-c3ccc(F)cn3)nn21]

The title compound was prepared in a manner analogous to Intermediate 37 Steps A-B except using (R)-6-methyl-5,6-dihydro-4H-pyrrolo[1,2-c][1,2,3]oxadiazol-7-ium-3-olate (Intermediate 4) instead of 6,7-dihydro-4H-[1,2,3]oxadiazolo[4,3-c][1,4]oxazin-8-ium-3-olate (Intermediate 2), microwave irradiation at 150° C. for 1.5 h rather than conventional heating in Step A. DCM was used instead of DMF in Step B. MS (ESI): mass calcd. for C₁₂H₁₁BrFN₃ 295.0 m/z found 295.7 [M+H]⁺.

### Intermediate 46: Racemic (3bS,4aR)-3-bromo-2-(5-fluoropyridin-2-yl)-3b,4,4a,5-tetrahydrocyclopropa[3,4]pyrrolo[1,2-b]pyrazole

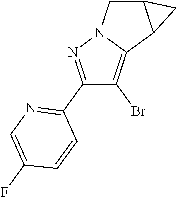 [SMILES: Fc1ccc(-c2nn3c(c2Br)C2CC2C3)nc1]

The title compound was prepared in a manner analogous to Intermediate 37, except using racemic (3bS,4aR)-3b,4,4a,5-tetrahydrocyclopropa[3,4]pyrrolo[1,2-c][1,2,3]oxadiazol-6-ium-3-olate (Intermediate 6) instead of 6,7-dihydro-4H-[1,2,3]oxadiazolo[4,3-c][1,4]oxazin-8-ium-3-olate (Intermediate 2). MS (ESI): mass calcd. for C₁₂H₉BrFN₃, 293.0; m/z found, 294.0 [M+H]⁺.

### Intermediate 47: (4aR,5aR)-3-Bromo-2-(5-fluoropyridin-2-yl)-4,4a,5,5a-tetrahydrocyclopropa[4,5]pyrrolo[1,2-b]pyrazole

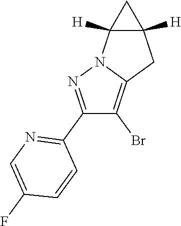 [SMILES: Fc1ccc(-c2nn3c(c2Br)C[C@H]2C[C@H]23)nc1]

The title compound was prepared in a manner analogous to Intermediate 37, except using (4aR,5aR)-4,4a,5,5a-tetrahydrocyclopropa[4,5]pyrrolo[1,2-c][1,2,3]oxadiazol-6-ium-3-olate (Intermediate 7) instead of 6,7-dihydro-4H-[1,2,3]oxadiazolo[4,3-c][1,4]oxazin-8-ium-3-olate (Intermediate 2). MS (ESI): mass calcd. for C₁₂H₉BrFN₃, 293.0; m/z found, 294.0 [M+H]⁺.

### Intermediate 48: (4aS,5aS)-3-Bromo-2-(5-fluoropyridin-2-yl)-4,4a,5,5a-tetrahydrocyclopropa[4,5]pyrrolo[1,2-b]pyrazole

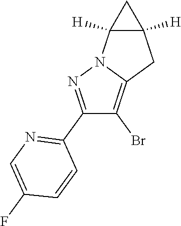 [SMILES: Fc1ccc(-c2nn3c(c2Br)C[C@@H]2C[C@@H]23)nc1]

The title compound was prepared in a manner analogous to Intermediate 37, except using (4aS,5aS)-4,4a,5,5a-tetrahydrocyclopropa[4,5]pyrrolo[1,2-c][1,2,3]oxadiazol-6-ium-3-olate (Intermediate 8) instead of 6,7-dihydro-4H-[1,2,3]oxadiazolo[4,3-c][1,4]oxazin-8-ium-3-olate (Intermediate 2). MS (ESI): mass calcd. for C₁₂H₉BrFN₃, 293.0; m/z found, 294.0 [M+H]⁺.

### Intermediate 49: 3-Bromo-2-(5-fluoropyridin-2-yl)-4,5,6,7-tetrahydropyrazolo[1,5-a]pyridine

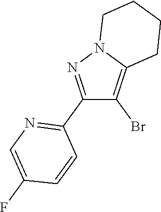 [SMILES: Fc1ccc(-c2nn3c(c2Br)CCCC3)nc1]

The title compound was prepared in a manner analogous to Intermediate 37, Steps A-B, except using 4,5,6,7-tetrahydro-[1,2,3]oxadiazolo[3,4-a]pyridin-8-ium-3-olate (Intermediate 9) instead of 6,6-dimethyl-6,7-dihydro-4H-[1,2,3]oxadiazolo[4,3-c][1,4]oxazin-8-ium-3-olate (Intermediate 17) in Step A. MS (ESI): mass calcd. for C₁₂H₁₁BrFN₃, 295.0; m/z found, 296.0 [M+H]⁺. ¹H NMR (400 MHz, CDCl₃): δ 8.57 (d, J=2.9 Hz, 1H), 8.00 (dd, J=8.8, 4.4 Hz, 1H), 7.45 (ddd, J=8.8, 8.1, 2.9 Hz, 1H), 4.21 (t, J=6.1 Hz, 2H), 2.76 (t, J=6.4 Hz, 2H), 2.14-2.01 (m, 2H), 2.00-1.85 (m, 2H).

### Intermediate 50: 3-Bromo-2-(3,5-difluoropyridin-2-yl)-4,5,6,7-tetrahydropyrazolo[1,5-a]pyridine

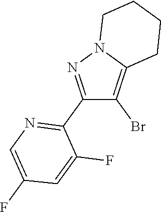 [SMILES: Fc1cnc(-c2nn3c(c2Br)CCCC3)c(F)c1]

The title compound was prepared in a manner analogous to Intermediate 37, Steps A-B, except using 4,5,6,7-tetrahydro-[1,2,3]oxadiazolo[3,4-a]pyridin-8-ium-3-olate (Intermediate 9) instead of 6,6-dimethyl-6,7-dihydro-4H-[1,2,3]oxadiazolo[4,3-c][1,4]oxazin-8-ium-3-olate (Intermediate 17) and 2-ethynyl-3,5-difluoropyridine instead of 2-ethynyl-5-fluoropyridine and also and microwave heating at 155° C. for 1 hr instead of conventional heating for 16 hrs in Step A. DCM was used instead of DMF in Step B. MS (ESI): mass calcd. for C₁₂H₁₀BrF₂N₃, 313.0; m/z found, 313.9 [M+H]⁺. ¹H NMR (400 MHz, DMSO-d₆) δ 8.62 (d, J=2.3 Hz, 1H), 8.12-8.01 (m, 1H), 4.13 (t, J=6.0 Hz, 2H), 2.69 (t, J=6.4 Hz, 2H), 2.04-1.96 (m, 2H), 1.91-1.82 (m, 2H) Intermediate 51: Racemic 3-Bromo-2-(4-fluorophenyl)-7-methyl-4,5,6,7-tetrahydropyrazolo[1,5-a]pyridine

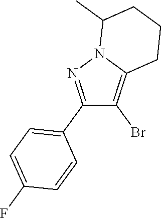 [SMILES: CC1CCCc2c(Br)c(-c3ccc(F)cc3)nn21]

The title compound was prepared in a manner analogous to Intermediate 37 Steps A-B except using Racemic 7-methyl-4,5,6,7-tetrahydro-[1,2,3]oxadiazolo[3,4-a]pyridin-8-ium-3-olate (Intermediate 10) instead of 6,7-dihydro-4H-[1,2,3]oxadiazolo[4,3-c][1,4]oxazin-8-ium-3-olate (Intermediate 2), 1-ethynyl-4-fluorobenzene instead of 2-ethynyl-5-fluoropyridine, and also microwave irradiation at 155° C. for 1.5 h rather than conventional heating. DCM was used instead of DMF in Step B. ¹H NMR (400 MHz, CDCl₃): δ 7.95-7.74 (m, 2H), 7.18-6.95 (m, 2H), 4.38-4.18 (m, 1H), 2.88-2.61 (m, 2H), 2.27-2.11 (m, 1H), 2.09-1.93 (m, 1H), 1.90-1.71 (m, 2H), 1.60 (d, J=6.6 Hz, 3H).

### Intermediate 52: 3-Bromo-6,6-difluoro-2-(4-fluorophenyl)-4,5,6,7-tetrahydropyrazolo[1,5-a]pyridine

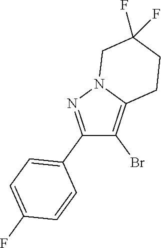 [SMILES: Fc1ccc(-c2nn3c(c2Br)CCC(F)(F)C3)cc1]

The title compound was prepared in a manner analogous to Intermediate 37 Steps A-B except using 6,6-difluoro-4,5,6,7-tetrahydro-[1,2,3]oxadiazolo[3,4-a]pyridin-8-ium-3-olate (Intermediate 11) instead of 6,7-dihydro-4H-[1,2,3]oxadiazolo[4,3-c][1,4]oxazin-8-ium-3-olate (Intermediate 2), 1-ethynyl-4-fluorobenzene instead of 2-ethynyl-5-fluoropyridine, diphenyl ether instead of xylenes and also microwave irradiation at 240° C. for 1 h rather than conventional heating. DCM was used instead of DMF in Step B. MS (ESI): mass calcd. for C₁₃H₁₀BrF₃N₂ 330.00 m/z found 330.9 [M+H]⁺.

### Intermediate 53: 3-Bromo-6,6-difluoro-2-(5-fluoropyridin-2-yl)-4,5,6,7-tetrahydropyrazolo[1,5-a]pyridine

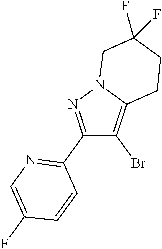 [SMILES: Fc1ccc(-c2nn3c(c2Br)CCC(F)(F)C3)nc1]

The title compound was prepared in a manner analogous to Intermediate 37, except using 6,6-difluoro-4,5,6,7-tetrahydro-[1,2,3]oxadiazolo[3,4-a]pyridin-8-ium-3-olate (Intermediate 11) instead of 6,7-dihydro-4H-[1,2,3]oxadiazolo[4,3-c][1,4]oxazin-8-ium-3-olate (Intermediate 2). MS (ESI): mass calcd. for C₁₂H₉BrF₃N₃, 331.0; m/z found, 332.0 [M+H]⁺.

### Intermediate 54: 3-Bromo-5,5-difluoro-2-(4-fluorophenyl)-4,5,6,7-tetrahydropyrazolo[1,5-a]pyridine

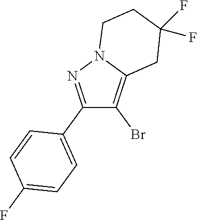 [SMILES: Fc1ccc(-c2nn3c(c2Br)CC(F)(F)CC3)cc1]

The title compound was made in a manner analogous to Intermediate 37, Step A-B except using 1-ethynyl-4-fluorobenzene instead of 2-ethynyl-5-fluoropyridine, 5,5-difluoro-4,5,6,7-tetrahydro-[1,2,3]oxadiazolo[3,4-a]pyridin-8-ium-3-olate (Intermediate 12) instead of 6,7-dihydro-4H-[1,2,3]oxadiazolo[4,3-c][1,4]oxazin-8-ium-3-olate (Intermediate 2), diphenyl ether instead of xylenes and also microwave irradiation at 240° C. for 2 h rather than conventional heating. DCM was used instead of DMF in Step B. LC-MS (ESI): mass calcd. for C₁₃H₁₀BrF₃N₂ 330.00 m/z found 330.9 [M+H]⁺.

### Intermediate 55: 3-Bromo-5,5-difluoro-2-(5-fluoropyridin-2-yl)-4,5,6,7-tetrahydropyrazolo[1,5-a]pyridine

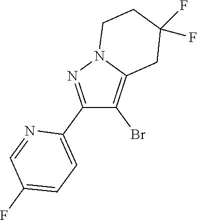 [SMILES: Fc1ccc(-c2nn3c(c2Br)CC(F)(F)CC3)nc1]

The title compound was prepared in a manner analogous to Intermediate 37 Steps A-B except using 5,5-difluoro-4,5,6,7-tetrahydro-[1,2,3]oxadiazolo[3,4-a]pyridin-8-ium-3-olate (Intermediate 12) instead of 6,7-dihydro-4H-[1,2,3]oxadiazolo[4,3-c][1,4]oxazin-8-ium-3-olate (Intermediate 2) and also microwave irradiation at 240° C. for 1 h rather than conventional heating. DCM was used instead of DMF in Step B. MS (ESI): mass calcd. for C₁₂H₉BrF₃N₃ 331.0 m/z, found 331.9 [M+H]⁺.

### Intermediate 56: 3-Bromo-2-(5-fluoropyridin-2-yl)-5,5-dimethyl-4,5,6,7-tetrahydropyrazolo[1,5-a]pyridine

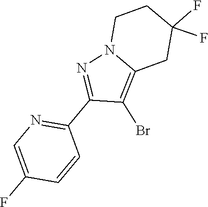 [SMILES: Fc1ccc(-c2nn3c(c2Br)CC(F)(F)CC3)nc1]

The title compound was prepared in a manner analogous to Intermediate 37 Steps A-B except using 5,5-dimethyl-4,5,6,7-tetrahydro-[1,2,3]oxadiazolo[3,4-a]pyridin-8-ium-3-olate (Intermediate 13) instead of 6,7-dihydro-4H-[1,2,3]oxadiazolo[4,3-c][1,4]oxazin-8-ium-3-olate (Intermediate 2), 1-ethynyl-4-fluorobenzene instead of 2-ethynyl-5-fluoropyridine, diphenyl ether instead of xylenes and also microwave irradiation at 240° C. for 1 h rather than conventional heating. DCM was used instead of DMF in Step B. LC-MS (ESI): mass calcd. for C₁₄H₁₅BrFN₃ 323.0 m/z, found 323.8 [M+2H]⁺.

### Intermediate 57: 3-Bromo-2-(5-fluoropyridin-2-yl)-6,6-dimethyl-4,5,6,7-tetrahydropyrazolo[1,5-a]pyridine

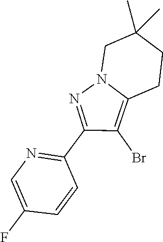 [SMILES: CC1(C)CCc2c(Br)c(-c3ccc(F)cn3)nn2C1]

Step A: (2-(5-Fluoropyridin-2-yl)-6-methyl-4,5,6,7-tetrahydropyrazolo[1,5-a]pyridin-6-yl)methyl methanesulfonate. MsCl (1.78 g, 15.5 mmol) was added in portions to a 0° C. (ice/water) solution consisting of (2-(5-fluoropyridin-2-yl)-6-methyl-4,5,6,7-tetrahydropyrazolo[1,5-a]pyridin-6-yl)methanol (Intermediate 59, 500 mg, 1.91 mmol), Et₃N (1.52 mL, 10.9 mmol), and dichloromethane (10 mL). The resultant mixture was stirred at room-temperature under N₂ for 4 h. The reaction mixture was quenched with sat. aq. NaHCO₃(5 mL) and extracted with dichloromethane (20 mL×3). The combined organic extracts were dried over anhydrous Na₂SO₄, filtered, and concentrated to dryness under reduced pressure. The resulting residue was purified by FCC (SiO₂, eluent: petroleum ether:ethyl acetate=1:0 to 1:5) to afford the title compound (665 mg, 99%) as a yellow solid. MS (ESI): mass calcd. for C₁₅H₁₈FN₃O₃S 339.1 m/z, found 340.1 [M+H]⁺.

Step B: 2-(5-Fluoropyridin-2-yl)-6,6-dimethyl-4,5,6,7-tetrahydropyrazolo[1,5-a]pyridine. NaI (537 mg, 3.58 mmol) was added to a solution consisting of (2-(5-fluoropyridin-2-yl)-6-methyl-4,5,6,7-tetrahydropyrazolo[1,5-a]pyridin-6-yl)methyl methanesulfonate (450 mg, 1.33 mmol), Zn dust (460 mg, 7.03 mmol), and HMPA (8 mL). The resulting mixture was stirred for 72 h at 125° C. The reaction mixture was gradually warmed to ambient temperature, then quenched with H₂O (5 mL), and extracted with dichloromethane (20 mL×3). The combined organic extracts were dried over anhydrous Na₂SO₄, filtered, and concentrated to dryness under reduced pressure. The resulting residue was purified by FCC (SiO₂, eluent: petroleum ether:ethyl acetate=1:0 to 1:5) to afford the title compound (250 mg, 76%) as a yellow solid. MS (ESI): mass calcd. for C₁₄H₁₆FN₃ 245.1 m/z, found 246.3 [M+H]⁺.

Step 3: 3-Bromo-2-(5-fluoropyridin-2-yl)-6,6-dimethyl-4,5,6,7-tetrahydropyrazolo[1,5-a]pyridine. The title compound was prepared in a manner analogous to Intermediate 37, Step B except using 2-(5-fluoropyridin-2-yl)-6,6-dimethyl-4,5,6,7-tetrahydropyrazolo[1,5-a]pyridine instead of 2-(5-fluoropyridin-2-yl)-6,7-dihydro-4H-pyrazolo[5,1-c][1,4]oxazine (Intermediate 37, Step A) in Step A and DCM instead of DMF in Step B. LC-MS (ESI): mass calcd. for C₁₄H₁₅BrFN₃ 323.0 m/z, found 323.9 [M+H]⁺. ¹H NMR (400 MHz, DMSO-d₆) δ 8.62 (d, J=3.1 Hz, 1H), 7.90 (dd, J=4.6, 8.8 Hz, 1H), 7.78 (d, J=3.0, 8.8 Hz, 1H), 3.85 (s, 2H), 2.69 (t, J=6.7 Hz, 2H), 1.69 (t, J=6.7 Hz, 2H), 1.03 (s, 6H).

### Intermediate 58: Methyl 2-(5-fluoropyridin-2-yl)-4,5,6,7-tetrahydropyrazolo[1,5-a]pyridine-6-carboxylate

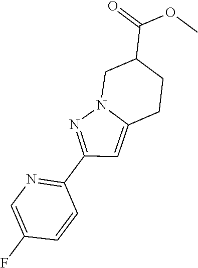 [SMILES: COC(=O)C1CCc2cc(-c3ccc(F)cn3)nn2C1]

Step A: 5-(Methoxycarbonyl)piperidine-2-carboxylic acid. 5-(Methoxycarbonyl)picolinic acid (25.0 g, 138 mmol), wet Pd/C (5 g, 10%), AcOH (500 mL), MeOH (62.5 mL) were added to a 1 L high pressure flask. The resultant mixture was stirred under H₂ (10 atm) at room-temperature for 24 hours. The suspension was filtered through a pad of Celite® and the pad was washed with ethyl acetate (100 mL). The filtrate was concentrated to dryness under reduced pressure to afford the title product (15 g, 57%), which was used in the next step without further purification.

Step B: 6-(Methoxycarbonyl)-4,5,6,7-tetrahydro-[1,2,3]oxadiazolo[3,4-a]pyridin-8-ium-3-olate. The title compound was prepared in a manner analogous to Intermediate 2, Step A-B expect using 5-(methoxycarbonyl)piperidine-2-carboxylic acid instead of morpholine-3-carboxylic acid in Step A. The title compound was used in the subsequent step without further purification.

Step C: Methyl 2-(5-fluoropyridin-2-yl)-4,5,6,7-tetrahydropyrazolo[1,5-a]pyridine-6-carboxylate. To a solution of 6-(methoxycarbonyl)-4,5,6,7-tetrahydro-[1,2,3]oxadiazolo[3,4-a]pyridin-8-ium-3-olate (7.52 g, 37.9 mmol) in xylenes (80 mL) was added 2-ethynyl-5-fluoropyridine (11.5 g, 95.0 mmol). The reaction was stirred at 145° C. for 5 hours. The reaction mixture was cooled to room-temperature. The reaction mixture was concentrated to dryness under reduced pressure to give the title compound. Purification (FCC, SiO₂, eluent: dichlormethane:methanol=1:0 to 10:1) afforded the title compound (8 g, 76%) as a yellow solid. MS (ESI): mass calcd. for C₁₅H₁₆FN₃O₂ 275.1 m/z, found 275.9 [M+H]⁺.

### Intermediate 59: (2-(5-Fluoropyridin-2-yl)-6-methyl-4,5,6,7-tetrahydropyrazolo[1,5-a]pyridin-6-yl)methanol

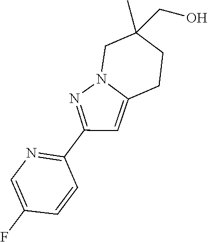 [SMILES: CC1(CO)CCc2cc(-c3ccc(F)cn3)nn2C1]

Step A: Methyl 2-(5-fluoropyridin-2-yl)-6-methyl-4,5,6,7-tetrahydropyrazolo[1,5-a]pyridine-6-carboxylate. Lithium bis(trimethylsilyl)amide (22.3 mL, 1 M in THF, 22.3 mmol) was added dropwise to a −70° C. (dry ice/ethanol) solution consisting of methyl 2-(5-fluoropyridin-2-yl)-4,5,6,7-tetrahydropyrazolo[1,5-a]pyridine-6-carboxylate (Intermediate 58, 2.45 g, 8.9 mmol) and THF (50 mL). The resultant mixture was stirred for 50 minutes at −70° C. and then treated with iodomethane (11 g, 71.1 mmol) dropwise at −70° C. The resultant mixture was stirred for another 4 h. The reaction mixture was gradually warmed to ambient temperature, then poured into sat. NH₄Cl (50 mL) and extracted with ethyl acetate (60 mL×3). The combined organic extracts were washed with brine (30 mL), dried over anhydrous Na₂SO₄, filtered, and concentrated to dryness under reduced pressure. The resulting residue was purified by FCC (SiO₂, eluent: petroleum ether:ethyl acetate=1:0 to 1:1) to afford the title compound (1.8 g) as a yellow oil. MS (ESI): mass calcd. for C₁₅H₁₆FN₃O₂ 289.1 m/z, found 290.3 [M+H]⁺.

Step B: (2-(5-Fluoropyridin-2-yl)-6-methyl-4,5,6,7-tetrahydropyrazolo[1,5-a]pyridin-6-yl)methanol. LiBH₄ (1.76 g, 81.0 mmol) was added in portions to a 0° C. (ice/water) mixture consisting of methyl 2-(5-fluoropyridin-2-yl)-6-methyl-4,5,6,7-tetrahydropyrazolo[1,5-a]pyridine-6-carboxylate (1.8 g, 6.22 mmol) and THF (35 mL). The resultant mixture was stirred at room-temperature for 16 h. The reaction mixture was quenched with water (30 mL) and extracted with ethyl acetate (50 mL×3). The combined organic extracts were dried over Na₂SO₄, filtered, and concentrated to dryness under reduced pressure to afford the title compound (1.5 g) as a yellow solid, which was used in the next step without further purification. ¹H NMR (400 MHz, DMSO-d₆) δ 8.53 (m, 1H), 7.91 (m, 1H), 7.71 (m, 1H), 6.53 (s, 1H), 4.85 (m, 1H), 3.96 (m, 1H), 3.78 (d, J=12.6 Hz, 1H), 3.31-3.28 (m, 2H), 2.83-2.76 (m, 2H), 1.79-1.69 (m, 1H), 1.62-1.53 (m, 1H), 0.98 (s, 3H).

### Intermediate 60: 3-Bromo-6-(fluoromethyl)-2-(5-fluoropyridin-2-yl)-6-methyl-4,5,6,7-tetrahydropyrazolo[1,5-a]pyridine

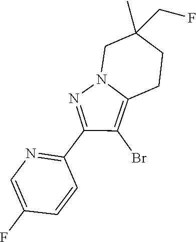 [SMILES: CC1(CF)CCc2c(Br)c(-c3ccc(F)cn3)nn2C1]

Step A: 6-(Fluoromethyl)-2-(5-fluoropyridin-2-yl)-6-methyl-4,5,6,7-tetrahydropyrazolo[1,5-a]pyridine. Tetrabutylammonium fluoride trihydrate (2.22 g, 7.04 mmol) was added to a solution consisting of (2-(5-fluoropyridin-2-yl)-6-methyl-4,5,6,7-tetrahydropyrazolo[1,5-a]pyridin-6-yl)methyl methanesulfonate (Intermediate 57 product from Step A, 450 mg, 1.33 mmol) and methyl ethyl ketone (10 mL). The resultant mixture was stirred at 90° C. for 24 h. The reaction mixture was gradually warmed to ambient temperature, then quenched with sat. NH₄Cl (8 mL) and extracted with ethyl acetate (25 mL×3). The combined organic extracts were dried over anhydrous Na₂SO₄, filtered, and concentrated to dryness under reduced pressure. The resulting residue was purified by FCC (SiO₂, eluent: petroleum ether:ethyl acetate=1:0 to 0:1) to afford the title compound (125 mg, 35%) as a yellow solid. MS (ESI): mass calcd. for C₁₄H₁₅F₂N₃ 263.1 m/z, found 263.9 [M+H]⁺.

Step B: 3-Bromo-6-(fluoromethyl)-2-(5-fluoropyridin-2-yl)-6-methyl-4,5,6,7-tetrahydropyrazolo[1,5-a]pyridine. The title compound was prepared in a manner analogous to Intermediate 7, Step B except using 6-(fluoromethyl)-2-(5-fluoropyridin-2-yl)-6-methyl-4,5,6,7-tetrahydropyrazolo[1,5-a]pyridine instead of 2-(5-fluoropyridin-2-yl)-6,7-dihydro-4H-pyrazolo[5,1-c][1,4]oxazine and DCM instead of DMF. MS (ESI): mass calcd. for C₁₄H₁₄BrF₂N₃ 341.0 m/z, found 341.9 [M+H]⁺. ¹H NMR (400 MHz, DMSO-d₆) δ 8.63 (d, J=2.9 Hz, 1H), 7.98-7.68 (m, 2H), 4.52-4.27 (m, 2H), 4.09-3.91 (m, 2H), 2.81-2.69 (m, 2H), 1.89-1.70 (m, 2H), 1.06 (s, 3H).

### Intermediate 61: 3-Bromo-6-fluoro-2-(5-fluoropyridin-2-yl)-6-(methoxymethyl)-4,5,6,7-tetrahydropyrazolo[1,5-a]pyridine

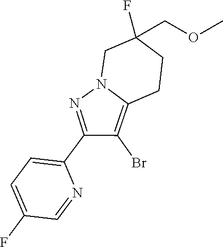 [SMILES: COCC1(F)CCc2c(Br)c(-c3ccc(F)cn3)nn2C1]

Step A: Methyl 6-fluoro-2-(5-fluoropyridin-2-yl)-4,5,6,7-tetrahydropyrazolo[1,5-a]pyridine-6-carboxylate. LiHMDS (4.4 mL, 4.34 mmol, 1M in THF) was added dropwise to a cooled (−70° C.; dry ice/ethanol) solution consisting of methyl 2-(5-fluoropyridin-2-yl)-4,5,6,7-tetrahydropyrazolo[1,5-a]pyridine-6-carboxylate (Intermediate 58, 800 mg, 2.91 mmol) and THF (15 mL). The resultant mixture was stirred at −70° C. for 50 minutes and then treated with NFSI (1.83 g, 5.80 mmol) by dropwise at −70° C. The resultant mixture was stirred for 5 hours. The reaction mixture was gradually warmed to room-temperature, then poured into sat·NH₄Cl (20 mL) and extracted with ethyl acetate (30 mL×3). The combined organic extracts were washed with brine (10 mL), dried over anhydrous Na₂SO₄, filtered, and concentrated to dryness under reduced pressure. The resulting residue was purified by FCC (SiO₂, eluent: petroleum ether:ethyl acetate=1:0 to 1:1) to afford the title compound (850 mg) as a yellow oil. MS (ESI): mass calcd. for C₁₄H₁₃F₂N₃O₂ 293.1 m/z, found 294.1 [M+H]⁺.

Step B: (6-Fluoro-2-(5-fluoropyridin-2-yl)-4,5,6,7-tetrahydropyrazolo[1,5-a]pyridin-6-yl)methanol. LiBH₄ (0.821 g, 37.68 mmol) was added in portions to a mixture of methyl 6-fluoro-2-(5-fluoropyridin-2-yl)-4,5,6,7-tetrahydropyrazolo[1,5-a]pyridine-6-carboxylate (0.85 g, 2.90 mmol) and THF (15 mL) at 0° C. The mixture was stirred at room-temperature for 16 hours. The reaction mixture was poured into water (30 mL) and extracted with ethyl acetate (50 mL×3). The organic layer was dried over Na₂SO₄, filtered and concentrated to dryness under reduced pressure to give the title compound (0.7 g) as a yellow solid, which was used in the next step without further purification. MS (ESI): mass calcd. for C₁₃H₁₃F₂N₃O 265.1 m/z, found 265.9 [M+H]⁺.

Step C: 6-Fluoro-2-(5-fluoropyridin-2-yl)-6-(methoxymethyl)-4,5,6,7-tetrahydropyrazolo[1,5-a]pyridine. Sodium hydride in mineral oil (151 mg, 60% purity, 1.53 mmol) was added in portions to a 0° C. (ice/water) solution consisting of (6-fluoro-2-(5-fluoropyridin-2-yl)-4,5,6,7-tetrahydropyrazolo[1,5-a]pyridin-6-yl)methanol (500 mg, 1.89 mmol) and THF (10 mL). The resultant mixture was treated with Mel (2.68 g, 18.9 mmol) at 0° C. and then stirred for 1.5 hours. The reaction mixture was gradually warmed to room-temperature, then quenched with sat. NH₄Cl (10 mL) and extracted with ethyl acetate (20 mL×3). The mixture was concentrated to dryness under reduced pressure. The resulting residue was purified by FCC (SiO₂, eluent: petroleum ether:ethyl acetate=1:0 to 10:1) to afford the product (350 mg) as a yellow solid. MS (ESI): mass calcd. for C₁₄H₁₅F₂N₃O 279.1 m/z, found 279.9 [M+H]⁺.

Step D: 3-Bromo-6-fluoro-2-(5-fluoropyridin-2-yl)-6-(methoxymethyl)-4,5,6,7-tetrahydropyrazolo[1,5-a]pyridine. The title compound was prepared in a manner analogous to Intermediate 37, Step B except using 6-fluoro-2-(5-fluoropyridin-2-yl)-6-(methoxymethyl)-4,5,6,7-tetrahydropyrazolo[1,5-a]pyridine instead of 2-(5-fluoropyridin-2-yl)-6,7-dihydro-4H-pyrazolo[5,1-c][1,4]oxazine and 3-(5-fluoropyridin-2-yl)-6,7-dihydro-4H-pyrazolo[5,1-c][1,4]oxazine (Intermediate 37, Step A) and DCM instead of DMF. MS (ESI): mass calcd. for C₁₄H₁₄BrF₂N₃O 357.0, found 357.8 [M+H]⁺.

### Intermediate 62: 3-Bromo-2-(5-fluoropyridin-2-yl)-6-(methoxymethyl)-6-methyl-4,5,6,7-tetrahydropyrazolo[1,5-a]pyridine

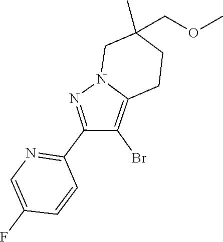 [SMILES: COCC1(C)CCc2c(Br)c(-c3ccc(F)cn3)nn2C1]

Step A: 2-(5-Fluoropyridin-2-yl)-6-(methoxymethyl)-6-methyl-4,5,6,7-tetrahydropyrazolo[1,5-a]pyridine. Sodium hydride in mineral oil (153 mg, 60% purity, 3.83 mmol) was added in portions to a 0° C. (ice/water) solution consisting of (2-(5-fluoropyridin-2-yl)-6-methyl-4,5,6,7-tetrahydropyrazolo[1,5-a]pyridin-6-yl)methanol (Intermediate 59, 500 mg, 1.91 mmol) and THF (15 mL). The resultant mixture was treated with Mel (3.40 g, 23.9 mmol) dropwise and stirred for 1.5 hours. The reaction mixture was gradually warmed to room-temperature, then quenched with sat. NH₄Cl (10 mL) and extracted with ethyl acetate (20 mL×3). The organic extracts were concentrated to dryness under reduced pressure. The resulting residue was purified by FCC (SiO₂, eluent: petroleum ether:ethyl acetate=1:0 to 10:1) to afford the product (320 mg, purity 96.41%, yield 59%) as a yellow solid. LC-MS (ESI): mass calcd. for C₁₅H₁₈FN₃O 275.3 m/z found 276.0 [M+H]⁺.

Step B: 3-Bromo-2-(5-fluoropyridin-2-yl)-6-(methoxymethyl)-6-methyl-4,5,6,7-tetrahydropyrazolo[1,5-a]pyridine. The title compound was prepared in a manner analogous to Intermediate 37, Step B except using 2-(5-fluoropyridin-2-yl)-6-(methoxymethyl)-6-methyl-4,5,6,7-tetrahydropyrazolo[1,5-a]pyridine (Intermediate 62, Step A) instead of 2-(5-fluoropyridin-2-yl)-6,7-dihydro-4H-pyrazolo[5,1-c][1,4]oxazine and 3-(5-fluoropyridin-2-yl)-6,7-dihydro-4H-pyrazolo[5,1-c][1,4]oxazine (Intermediate 37, Step A) and DCM instead of DMF. LC-MS (ESI): mass calcd. for C₁₅H₁₇BrFN₃O 353.05 m/z found 353.8 [M+H]⁺.

### Intermediate 63: Racemic (5aR,6aS)-3-bromo-2-(5-fluoropyridin-2-yl)-5,5a,6,6a-tetrahydro-4H-cyclopropa[e]pyrazolo[1,5-a]pyridine

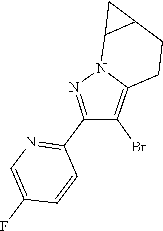 [SMILES: Fc1ccc(-c2nn3c(c2Br)CCC2CC23)nc1]

The title compound was prepared in a manner analogous to 3-bromo-2-(5-fluoropyridin-2-yl)-6,7-dihydro-4H-pyrazolo[5,1-c][1,4]oxazine (Intermediate 37), except using racemic (5aR,6aS)-5,5a,6,6a-tetrahydro-4H-cyclopropa[e][1,2,3]oxadiazolo[3,4-a]pyridin-7-ium-3-olate (Intermediate 14) instead of 6,7-dihydro-4H-[1,2,3]oxadiazolo[4,3-c][1,4]oxazin-8-ium-3-olate (Intermediate 2). MS (ESI): mass calcd. for C₁₃H₁₁BrFN₃, 307.0; m/z found, 308.0 [M+H]⁺.

### Intermediate 64: Racemic (5aR,6aS)-3-bromo-6,6-difluoro-2-(5-fluoropyridin-2-yl)-5,5a,6,6a-tetrahydro-4H-cyclopropa[e]pyrazolo[1,5-a]pyridine

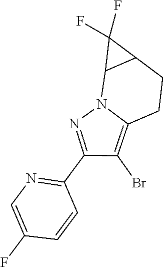 [SMILES: Fc1ccc(-c2nn3c(c2Br)CCC2C3C2(F)F)nc1]

The title compound was prepared in a manner analogous to 3-bromo-2-(5-fluoropyridin-2-yl)-6,7-dihydro-4H-pyrazolo[5,1-c][1,4]oxazine (Intermediate 37), except using racemic (5aR,6aS)-6,6-difluoro-5,5a,6,6a-tetrahydro-4H-cyclopropa[e][1,2,3]oxadiazolo[3,4-a]pyridin-7-ium-3-olate (Intermediate 15) instead of 6,7-dihydro-4H-[1,2,3]oxadiazolo[4,3-c][1,4]oxazin-8-ium-3-olate (Intermediate 2). MS (ESI): mass calcd. for C₁₃H₉BrF₃N₃, 343.0; m/z found, 344.0 [M+H]⁺.

### Intermediate 65: 3-Bromo-2-(4-fluorophenyl)-6,7-dihydro-4H-pyrazolo[5,1-c][1,4]oxazine

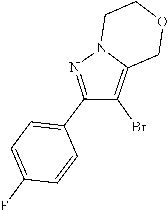 [SMILES: Fc1ccc(-c2nn3c(c2Br)COCC3)cc1]

Step A. 2-(4-Fluorophenyl)-6,7-dihydro-4H-pyrazolo[5,1-c][1,4]oxazine. To a solution of potassium phosphate tribasic (75 mg, 0.35 mmol) in H₂O (0.7 mL) was added 1-bromo-4-fluorobenzene (0.02 mL, 0.18 mmol), 2-(4,4,5,5-tetramethyl-1,3,2-dioxaborolan-2-yl)-6,7-dihydro-4H-pyrazolo[5,1-c][1,4]oxazine (Intermediate 27, 44 mg, 0.18 mmol), XPhos Pd G3 (6 mg, 0.007 mmol), and THF (0.7 mL). The reaction vial was capped and degassed by sparging with N₂. The mixture was heated to 50° C. for 16 h. The reaction mixture was cooled, diluted with H₂O (1 mL), and extracted with EtOAc (3×5 mL). The combined organics were dried (Na₂SO₄) and filtered. Purification by chromatography (silica gel, 0-100% EtOAc/hexanes) afforded 16 mg (42%) of the title compound. MS (ESI): mass calcd. for C₁₂H₁₁FN₂O, 218.1; m/z found, 219.1 [M+H]⁺. ¹H NMR (400 MHz, CDCl₃): δ 7.91-7.79 (m, 3H), 7.13 (t, J=8.69 Hz, 3H), 6.33 (s, 1H), 4.90 (s, 2H), 4.22-4.14 (m, 4H).

Step B. 3-Bromo-2-(4-fluorophenyl)-6,7-dihydro-4H-pyrazolo[5,1-c][1,4]oxazine. The title compound was prepared in a manner analogous to Intermediate 37, Step B using 2-(4-fluorophenyl)-6,7-dihydro-4H-pyrazolo[5,1-c][1,4]oxazine instead of 2-(5-fluoropyridin-2-yl)-6,7-dihydro-4H-pyrazolo[5,1-c][1,4]oxazine and DCM instead of DMF. MS (ESI): mass calcd. for C₁₂H₁₀BrFN₂O, 296.0; m/z found, 298.6 [M+H]⁺.

### Intermediate 66: 3-Bromo-2-(4-chlorophenyl)-6,7-dihydro-4H-pyrazolo[5,1-c][1,4]oxazine

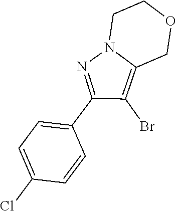 [SMILES: Clc1ccc(-c2nn3c(c2Br)COCC3)cc1]

The title compound was prepared in a manner analogous to Intermediate 37 Steps A-B, except using 1-chloro-4-ethynylbenzene instead of 2-ethynyl-5-fluoropyridine, and microwave irradiation at 150° C. for 1 hours instead of conventional heating in Step A; and DCM instead of DMF in Step B. ¹H NMR (400 MHz, DMSO-d₆) δ 7.86-7.81 (m, 2H), 7.56-7.51 (m, 2H), 4.75 (s, 2H), 4.18-4.13 (m, 2H), 4.13-4.07 (m, 2H).

### Intermediate 67: 3-Bromo-2-(4-chloro-3-fluorophenyl)-6,7-dihydro-4H-pyrazolo[5,1-c][1,4]oxazine

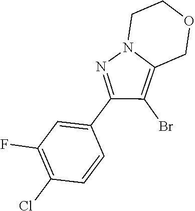 [SMILES: Fc1cc(-c2nn3c(c2Br)COCC3)ccc1Cl]

The title compound was prepared in a manner analogous to Intermediate 37 Steps A-B, except using 1-chloro-4-ethynyl-2-fluorobenzene instead of 2-ethynyl-5-fluoropyridine, and microwave irradiation at 150° C. for 1 hours instead of conventional heating in Step A; and DCM instead of DMF in Step B. ¹H NMR (400 MHz, DMSO-d₆): δ 7.80-7.75 (m, 1H), 7.74-7.68 (m, 2H), 4.76 (s, 2H), 4.20-4.14 (m, 2H), 4.14-4.07 (m, 2H) Intermediate 68: 3-Bromo-2-(5-fluoropyridin-3-yl)-6,7-dihydro-4H-pyrazolo[5,1-c][1,4]oxazine.

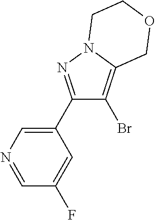 [SMILES: Fc1cncc(-c2nn3c(c2Br)COCC3)c1]

The title compound was prepared in a manner analogous to Intermediate 37 Steps A-B, except using 3-ethynyl-5-fluoropyridine instead of 2-ethynyl-5-fluoropyridine, and microwave irradiation at 150° C. for 1 hours instead of conventional heating in Step A; and DCM instead of DMF in Step B. MS (ESI): mass calcd. for C₁₁H₉BrFN₃O 297.0 m/z found 297.9 [M+H]⁺. ¹H NMR (400 MHz, DMSO-d₆) δ 8.90 (s, 1H), 8.63 (s, 1H), 8.05-8.00 (m, 1H), 4.78 (s, 2H), 4.23-4.18 (m, 2H), 4.14-4.10 (m, 2H).

### Intermediate 69: (R)-3-Bromo-2-(5-fluoropyridin-2-yl)-4-methyl-6,7-dihydro-4H-pyrazolo[5,1-c][1,4]oxazine

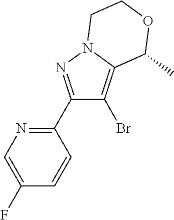 [SMILES: C[C@H]1OCCn2nc(-c3ccc(F)cn3)c(Br)c21]

The title compound was prepared in a manner analogous to Intermediate 37, Steps A-B, except using (R)-4-methyl-6,7-dihydro-4H-[1,2,3]oxadiazolo[4,3-c][1,4]oxazin-8-ium-3-olate (Intermediate 16) instead of 6,6-dimethyl-6,7-dihydro-4H-[1,2,3]oxadiazolo[4,3-c][1,4]oxazin-8-ium-3-olate (Intermediate 17) in Step A. ¹H NMR (400 MHz, CDCl₃): δ 8.58 (d, J=3.00 Hz, 1H) 8.00-7.93 (m, 1H) 7.51-7.44 (m, 1H) 4.97 (q, J=6.67 Hz, 1H) 4.32-4.18 (m, 3H) 4.04-3.95 (m, 1H) 1.72 (d, J=6.63 Hz, 3H).

### Intermediate 70: 3-Bromo-2-(4-fluorophenyl)-6-methyl-6,7-dihydro-4H-pyrazolo[5,1-c][1,4]oxazine

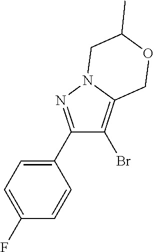 [SMILES: CC1Cn2nc(-c3ccc(F)cc3)c(Br)c2CO1]

Step A: (3-(4-Fluorophenyl)-1H-pyrazol-5-yl)methanol. LiAlH₄ (1.5 g, 39.5 mmol) was added in portions to a 250 mL three-necked round-bottomed flask containing a 0° C. (ice/water) solution consisting of ethyl 3-(4-fluorophenyl)-1H-pyrazole-5-carboxylate (Intermediate 33, 5.4 g, 23 mmol) and THF (50 mL) under N₂. The mixture was stirred under N₂ at room-temperature for 16 hours. The reaction mixture was quenched with H₂O (2 mL) and then 15% NaOH (aq., 2 mL). The resultant mixture was filtered through Celite® pad and the filter cake washed with ethyl acetate (40 mL×3). The filtrate was washed with brine (50 mL×2), dried over anhydrous MgSO₄, filtered, and concentrated to obtain the product, which was further purified by FCC (SiO₂, eluent: petroleum ether:ethyl acetate=1:0 to 0:1) to afford the title compound (4 g, 90%) as white solid. MS (ESI): mass calcd. for C₁₀H₉FN₂O 192.1 m/z found 193.2 [M+H]⁺. ¹H NMR (400 MHz, DMSO-d₆) δ 13.09-12.61 (m, 1H), 7.79 (d, J=6.0 Hz, 2H), 7.34-7.20 (m, 2H), 6.67-6.46 (m, 1H), 5.31 (br s, 1H), 4.55-4.36 (m, 2H).

Step B: (R)-1-(3-(4-Fluorophenyl)-5-(hydroxymethyl)-1H-pyrazol-1-yl)propan-2-ol. Cs₂CO₃ (4 g, 12.3 mmol) was added to a mixture consisting of (3-(4-fluorophenyl)-1H-pyrazol-5-yl)methanol (2 g, 10.4 mmol) and (R)-2-methyloxirane (6 g, 103.3 mmol). The resultant mixture was stirred for 12 hours at room-temperature. The suspension was filtered through a pad of Celite® and the pad washed with ethyl acetate (20 mL). The filtrate was concentrated to dryness under reduced pressure to give the product (1.9 g, crude) as a colorless oil, which was used in the next step without further purification. MS (ESI): mass calcd. for C₁₃H₁₅FN₂O₂ 250. 1 m/z found 251.2 [M+H]⁺.

Step C: 2-(4-Fluorophenyl)-6-methyl-6,7-dihydro-4H-pyrazolo[5,1-c][1,4]oxazine. (R)-1-(3-(4-fluorophenyl)-5-(hydroxymethyl)-1H-pyrazol-1-yl)propan-2-ol (1.9 g, 7.59 mmol) and conc. H₂SO₄ (15 mL) were added to a 100 mL round-bottomed flask. The reaction mixture was stirred at 90° C. for 16 hours. The reaction mixture was carefully added to 150 mL water cooled with ice, then the mixture was adjusted to pH=7-8 with 15% aq. NaOH, which was extracted with ethyl acetate (100 mL×3). The combined organic layers were dried over anhydrous Na₂SO₄, filtered, and concentrated to dryness under reduced pressure. The resulting residue was purified by FCC (SiO₂, eluent: petroleum ether:ethyl acetate=1:0 to 3:1) to afford the title compound (800 mg, 92%) as a white solid. MS (ESI): mass calcd. for C₁₃H₁₃FN₂O 232.1 m/z found 233.2 [M+H]⁺.

Step D: 3-Bromo-2-(4-fluorophenyl)-6-methyl-6,7-dihydro-4H-pyrazolo[5,1-c][1,4]oxazine. The title compound was prepared in a manner analogous to Intermediate 37, Step B, except using 2-(4-fluorophenyl)-6-methyl-6,7-dihydro-4H-pyrazolo[5,1-c][1,4]oxazine instead of 2-(5-fluoropyridin-2-yl)-6,7-dihydro-4H-pyrazolo[5,1-c][1,4]oxazine (Intermediate 37, Step A) and DCM instead of DMF. MS (ESI): mass calcd. for C₁₃H₁₂BrFN₂O 310.0 m/z found 311.1 [M+H]⁺.

### Intermediate 71: (*R)-3-Bromo-2-(4-fluorophenyl)-6-methyl-6,7-dihydro-4H-pyrazolo[5,1-c][1,4]oxazine

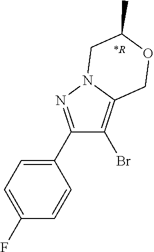 [SMILES: C[C@@H]1Cn2nc(-c3ccc(F)cc3)c(Br)c2CO1]

The title compound was purified from Intermediate 70. Purified by SFC Method A. The pure fractions were collected and the volatiles were removed under reduced pressure. The product was suspended in water (10 mL), the mixture frozen using dry ice/EtOH, and then lyophilized to dryness to afford the title compound (846 mg, 44%) as a white solid. MS (ESI): mass calcd. for C₁₃H₁₂BrFN₂O 310.01 m/z found 311.0 [M+H]⁺. ¹H NMR (400 MHz, DMSO-d₆) δ 7.87-7.78 (m, 2H), 7.36-7.25 (m, 2H), 4.87-4.81 (m, 1H), 4.77-4.66 (m, 1H), 4.22 (dd, J=2.9, 12.4 Hz, 1H), 4.13-4.02 (m, 1H), 3.79 (dd, J=10.7, 12.2 Hz, 1H), 1.31 (d, J=6.0 Hz, 3H).

### Intermediate 72: (*S)-3-Bromo-2-(4-fluorophenyl)-6-methyl-6,7-dihydro-4H-pyrazolo[5,1-c][1,4]oxazine

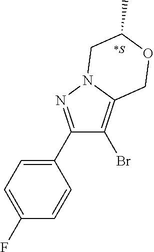 [SMILES: C[C@H]1Cn2nc(-c3ccc(F)cc3)c(Br)c2CO1]

The title compound was purified from Intermediate 70. Purified by SFC Method A. The pure fractions were collected and the volatiles were removed under reduced pressure. The product was suspended in water (10 mL), the mixture frozen using dry ice/EtOH, and then lyophilized to dryness to afford the title compound (756 mg, 39%) as white solid. MS (ESI): mass calcd. for C₁₃H₁₂BrFN₂O 310.0 m/z found 311.0 [M+H]⁺. ¹H NMR (400 MHz, DMSO-d₆) δ 7.87-7.80 (m, 2H), 7.34-7.26 (m, 2H), 4.87-4.82 (m, 1H), 4.75-4.68 (m, 1H), 4.26-4.19 (m, 1H), 4.12-4.03 (m, 1H), 3.83-3.74 (m, 1H), 1.32 (d, J=6.3 Hz, 3H).

### Intermediate 73: 3-Bromo-2-(5-fluoropyridin-2-yl)-6-methyl-6,7-dihydro-4H-pyrazolo[5,1-c][1,4]oxazine

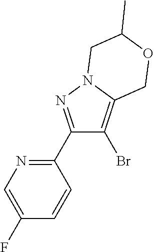 [SMILES: CC1Cn2nc(-c3ccc(F)cn3)c(Br)c2CO1]

Step A: (R)-1-(3-(5-Fluoropyridin-2-yl)-5-(hydroxymethyl)-1H-pyrazol-1-yl)propan-2-ol. Cs₂CO₃ (6.8 g, 20.9 mmol) was added to a mixture consisting of (3-(5-fluoropyridin-2-yl)-1H-pyrazol-5-yl)methanol (Intermediate 35, product from Step A, 2 g, 10.4 mmol) and (R)-2-methyloxirane (12 g, 206.6 mmol). The resultant mixture was stirred for 24 hours at room-temperature. The suspension was filtered through a pad of Celite® and the pad washed with ethyl acetate (30 mL). The filtrate was concentrated to dryness under reduced pressure to give the product (1.8 g, crude) as a yellow oil, which was used in the next step without further purification. MS (ESI): mass calcd. for C₁₂H₁₄FN₃O₂ 251.1 m/z found 252.1 [M+H]⁺.

Step B: 2-(5-Fluoropyridin-2-yl)-6-methyl-6,7-dihydro-4H-pyrazolo[5,1-c][1,4]oxazine. (R)-1-(3-(5-fluoropyridin-2-yl)-5-(hydroxymethyl)-1H-pyrazol-1-yl)propan-2-ol (1.8 g, 7.16 mmol) and conc. H₂SO₄ (15 mL) were added to a 100 mL round-bottomed flask. The reaction mixture was stirred at 90° C. for 16 hours. The reaction mixture was carefully added to 150 mL water cooled with ice, then the mixture was adjusted to pH=7-8 with 15% aq. NaOH, which was extracted with ethyl acetate (100 mL×3). The combined organic layers were dried over anhydrous Na₂SO₄, filtered, and concentrated to dryness under reduced pressure. The resulting residue was purified by FCC (SiO₂, eluent: petroleum ether:ethyl acetate=1:0 to 3:1) to afford the title compound (650 mg, 39%) as a white solid. MS (ESI): mass calcd. for C₁₂H₁₂FN₃O 233.10 m/z found 234.1 [M+H]⁺. ¹H NMR (400 MHz, CDCl₃) δ 8.46 (d, J=2.9 Hz, 1H), 7.94-7.86 (m, 1H), 7.47-7.40 (m, 1H), 6.57 (s, 1H), 5.01 (d, J=15.0 Hz, 1H), 4.82 (d, J=14.9 Hz, 1H), 4.25-4.17 (m, 1H), 4.09-4.01 (m, 1H), 3.94-3.85 (m, 1H), 1.43 (d, J=6.2 Hz, 3H).

Step C: 3-Bromo-2-(5-fluoropyridin-2-yl)-6-methyl-6,7-dihydro-4H-pyrazolo[5,1-c][1,4]oxazine. The title compound was prepared in a manner analogous to Intermediate 37, Step B, except using 2-(5-fluoropyridin-2-yl)-6-methyl-6,7-dihydro-4H-pyrazolo[5,1-c][1,4]oxazine instead of 2-(5-fluoropyridin-2-yl)-6,7-dihydro-4H-pyrazolo[5,1-c][1,4]oxazine (Intermediate 37, Step A) and DCM instead of DMF. MS (ESI): mass calcd. for C₁₂H₁₁BrFN₃O 311.0 m/z found 312.1 [M+H]⁺.

### Intermediate 74: (*R)-3-Bromo-2-(5-fluoropyridin-2-yl)-6-methyl-6,7-dihydro-4H-pyrazolo[5,1-c][1,4]oxazine

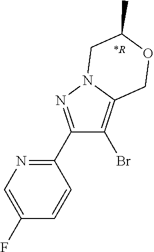 [SMILES: C[C@@H]1Cn2nc(-c3ccc(F)cn3)c(Br)c2CO1]

3-Bromo-2-(5-fluoropyridin-2-yl)-6-methyl-6,7-dihydro-4H-pyrazolo[5,1-c][1,4]oxazine (Intermediate 73, 640 mg) was purified by SFC Method A. The pure fractions were collected and the volatiles were removed under reduced pressure. The product was suspended in water (10 mL), the mixture frozen using dry ice/EtOH, and then lyophilized to dryness to afford the title compound (376 mg, 59%) as a white solid.

### Intermediate 75: (*S)-3-Bromo-2-(5-fluoropyridin-2-yl)-6-methyl-6,7-dihydro-4H-pyrazolo[5,1-c][1,4]oxazine

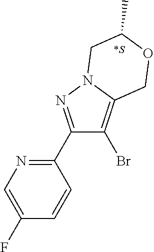 [SMILES: C[C@H]1Cn2nc(-c3ccc(F)cn3)c(Br)c2CO1]

3-Bromo-2-(5-fluoropyridin-2-yl)-6-methyl-6,7-dihydro-4H-pyrazolo[5,1-c][1,4]oxazine (Intermediate 73, 640 mg) was purified by SFC Method A. The pure fractions were collected and the volatiles were removed under reduced pressure. The product was suspended in water (10 mL), the mixture frozen using dry ice/EtOH, and then lyophilized to dryness to afford the title compound (182 mg, 29%) as white solid.

### Intermediate 76: 3-Bromo-2-(4-fluorophenyl)-7-methyl-6,7-dihydro-4H-pyrazolo[5,1-c][1,4]oxazine

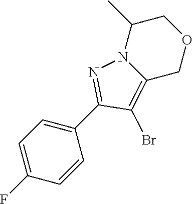 [SMILES: CC1COCc2c(Br)c(-c3ccc(F)cc3)nn21]

Step A: 2-(3-(4-Fluorophenyl)-5-(hydroxymethyl)-1H-pyrazol-1-yl)propan-1-ol. Ethyl 1-(1-ethoxy-1-oxopropan-2-yl)-3-(4-fluorophenyl)-1H-pyrazole-5-carboxylate (Intermediate 94, product from Step A, 4.9 g, 14.6 mmol) was dissolved in THF (250 mL) and cooled to −10° C. in an ice/MeOH bath under N₂ atm. The reaction mixture was stirred for 15 min, LiAlH₄(1M in THF, 22 mL, 22 mmol) was added dropwise over 15 min at −10° C., the reaction was allowed to slowly warm to 0° C. over 2 hours. The reaction was then diluted with ethyl acetate (150 mL) at 0° C. and stirred for 1 hour while warming to RT. The reaction was then quenched with saturated aqueous Rochelle's salt (100 mL) and then stirred vigorously for 2 hours before being transferred to a separatory funnel. The layers were separated, and the aqueous layer was extracted with more ethyl acetate (70 mL×3). The combined organic layers were dried over Na₂SO₄, filtered, and concentrated under reduced pressure to yield a clear colorless oil. MS (ESI): mass calcd. for C₁₃H₁₅FN₂O₂, 250.1; m/z found, 251.1 [M+H]⁺.

Step B: 3-Bromo-2-(4-fluorophenyl)-7-methyl-6,7-dihydro-4H-pyrazolo[5,1-c][1,4]oxazine. The title compound was made in a manner analogous to Intermediate 81, Steps C-D, except using 2-(3-(4-fluorophenyl)-5-(hydroxymethyl)-1H-pyrazol-1-yl)propan-1-ol (Intermediate 76, product from Step A) instead of 1-fluoro-3-(3-(4-fluorophenyl)-5-(hydroxymethyl)-1H-pyrazol-1-yl)propan-2-ol. MS (ESI): mass calcd. for C₁₃H₁₂BrFN₂O, 310.0; m/z found, 311.1 [M+H]⁺.

### Intermediate 77: 3-Bromo-6-ethyl-2-(5-fluoropyridin-2-yl)-6,7-dihydro-4H-pyrazolo[5,1-c][1,4]oxazine

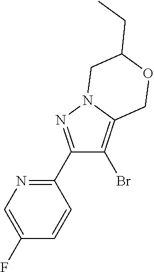 [SMILES: CCC1Cn2nc(-c3ccc(F)cn3)c(Br)c2CO1]

Step A. Ethyl 3-(5-fluoropyridin-2-yl)-1-(2-oxobutyl)-1H-pyrazole-5-carboxylate. 1-bromobutan-2-one (321 mg, 2.13 mmol) was added in portions to a 0° C. (ice/water) solution consisting of ethyl 3-(5-fluoropyridin-2-yl)-1H-pyrazole-5-carboxylate (Intermediate 34, 500 mg, 2.13 mmol), K₂CO₃ (441 mg, 3.19 mmol), and CH₃CN (6 mL). The resultant mixture was stirred at room-temperature for 4 hours. The mixture was concentrated to dryness under reduced pressure. The resulting residue was purified by FCC (SiO₂, eluent: petroleum ether:ethyl acetate=1:0 to 2:1) to afford the title compound (834 mg) as a white solid. LC-MS (ESI): mass calcd. for C₁₅H₁₆FN₃O₃ 305.12 m/z found 306.0 [M+H]⁺. ¹H NMR (400 MHz, CDCl₃): δ 8.48 (d, J=2.4 Hz, 1H), 7.93 (dd, J=4.4, 8.6 Hz, 1H), 7.58-7.36 (m, 2H), 5.50-5.30 (m, 2H), 4.31 (q, J=7.1 Hz, 2H), 2.52 (q, J=7.3 Hz, 2H), 1.36 (t, J=7.2 Hz, 3H), 1.13 (t, J=7.3 Hz, 3H).

Step B. 1-(3-(5-Fluoropyridin-2-yl)-5-(hydroxymethyl)-1H-pyrazol-1-yl)butan-2-ol. LiAlH₄ (264 mg, 6.96 mmol) was added in portions to a 0° C. (ice/water) solution consisting of ethyl 3-(5-fluoropyridin-2-yl)-1-(2-oxobutyl)-1H-pyrazole-5-carboxylate (709 mg, 2.32 mmol) and THF (8 mL) at a 100 mL three-necked round-bottomed flask under N₂. The resultant mixture was stirred for 4 hours. The reaction mixture was gradually warmed to room-temperature, then quenched with H₂O (1 mL) and aq·NaOH (15%, 1 mL) slowly. The mixture was filtered over Celite® pad and the filter cake washed with MeOH (20 mL×3). The filtrate was dried over anhydrous Na₂SO₄, filtered, and concentrated to dryness under reduced pressure to afford the title compound (872 mg, crude), which was used in the next step without further purification. LC-MS (ESI): mass calcd. for C₁₃H₁₆FN₃O₂ 265.12 m/z found 266.2 [M+H]⁺. ¹H NMR (400 MHz, CDCl₃): δ 8.46 (d, J=2.9 Hz, 1H), 7.91 (dd, J=4.2, 8.6 Hz, 1H), 7.43 (dt, J=2.9, 8.5 Hz, 1H), 6.61-6.53 (m, 1H), 5.08-4.95 (m, 1H), 4.79 (br d, J=14.8 Hz, 1H), 4.26-4.19 (m, 1H), 3.94-3.88 (m, 1H), 3.85-3.79 (m, 1H), 1.78-1.70 (m, 2H), 1.09-1.06 (m, 3H).

Step C. 6-Ethyl-2-(5-fluoropyridin-2-yl)-6,7-dihydro-4H-pyrazolo[5,1-c][1,4]oxazine. 1-(3-(5-Fluoropyridin-2-yl)-5-(hydroxymethyl)-1H-pyrazol-1-yl)butan-2-ol (632 mg, 2.38 mmol) was dissolved in H₃PO₄ (3 mL) and toluene (30 mL) at a 100 mL three-necked round-bottomed flask. The resultant mixture was stirred at 110° C. for 6 hours. The reaction mixture was gradually cooled to room-temperature. The pH was adjusted 7 with 1M NaOH (4 mL) and extracted with ethyl acetate (30 mL×3). The combined organic extracts were washed with water (30 mL×3), dried over anhydrous Na₂SO₄, filtered, and concentrated to dryness under reduced pressure to afford the title compound (420 mg), which was used in the next step without further purification. LC-MS (ESI): mass calcd. for C₁₃H₁₄FN₃O 247.11 m/z found 248.0 [M+H]⁺. ¹H NMR (400 MHz, DMSO-d₆) δ 8.51 (br d, J=2.9 Hz, 1H), 7.98-7.81 (m, 1H), 7.78-7.65 (m, 1H), 6.65-6.41 (m, 1H), 4.52 (br s, 1H), 4.11 (br d, J=5.1 Hz, 1H), 3.60 (br d, J=9.9 Hz, 1H), 3.10-2.97 (m, 2H), 1.48-1.31 (m, 2H), 0.90 (br t, J=7.3 Hz, 3H).

Step D. 3-Bromo-6-ethyl-2-(5-fluoropyridin-2-yl)-6,7-dihydro-4H-pyrazolo[5,1-c][1,4]oxazine. 6-Ethyl-2-(5-fluoropyridin-2-yl)-6,7-dihydro-4H-pyrazolo[5,1-c][1,4]oxazine (50 mg, 0.20 mmol) was dissolved in DMF (1 mL) in a 8 mL sealed tube. The mixture was cooled to 0° C. and then treated with a solution consisting of N-bromosuccinimide (36 mg, 0.20 mmol) and DMF (1 mL) by dropwise. The resultant mixture was stirred at room-temperature for 1 hour. The mixture was combined with additional batches then diluted with H₂O (15 mL). The resultant mixture was extracted with dichloromethane (10 mL×2). The combined organic extracts were dried over anhydrous Na₂SO₄, filtered, and concentrated to dryness under reduced pressure. The resulting residue was purified by FCC (SiO₂, eluent: petroleum ether:ethyl acetate=1:0 to 1:1) to afford the title compound (300 mg, 43%) as a yellow oil. LC-MS (ESI): mass calcd. for C₁₃H₁₃BrFN₃O 325.0 m/z found 326.0 [M+H]⁺.

### Intermediate 78: 3-Bromo-2-(5-fluoropyridin-2-yl)-6-isopropyl-6,7-dihydro-4H-pyrazolo[5,1-c][1,4]oxazine

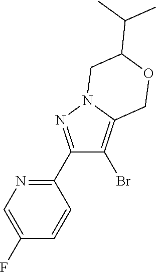 [SMILES: CC(C)C1Cn2nc(-c3ccc(F)cn3)c(Br)c2CO1]

Step A. 1-(3-(5-Fluoropyridin-2-yl)-5-(hydroxymethyl)-1H-pyrazol-1-yl)-3-methylbutan-2-one. 1-Bromo-3-methylbutan-2-one (1.8 g, 11 mmol) was added to a solution consisting of (3-(5-fluoropyridin-2-yl)-1H-pyrazol-5-yl)methanol (Intermediate 35, product from Step A, 2.2 g, crude), Cs₂CO₃ (7.4 g, 23 mmol), and CH₃CN (25 mL). The resultant mixture was stirred at room-temperature for 16 hours. The reaction mixture was diluted with H₂O (40 mL) and extracted with ethyl acetate (40 mL×2). The combined organic extracts were concentrated to dryness under reduced pressure. The resulting residue was purified by FCC (SiO₂, eluent: petroleum ether:ethyl acetate=1:0 to 1:9) to afford the title compound (900 mg) as a yellow oil. LC-MS (ESI): mass calcd. for C₁₄H₁₆FN₃O₂ 277.12 m/z found 278.4 [M+H]⁺.

Step B. 1-(3-(5-Fluoropyridin-2-yl)-5-(hydroxymethyl)-1H-pyrazol-1-yl)-3-methylbutan-2-ol. NaBH₄ (232 mg, 6.13 mmol) was added into a solution consisting of 1-(3-(5-fluoropyridin-2-yl)-5-(hydroxymethyl)-1H-pyrazol-1-yl)-3-methylbutan-2-one (850 mg, 3.07 mmol) and MeOH (10 mL). The resultant mixture was stirred at room-temperature for 2 hours before. The reaction mixture was quenched with sat. NH₄Cl (15 mL) and extracted with ethyl acetate (30 mL×3). The combined organic extracts were washed with brine (20 mL), dried over anhydrous Na₂SO₄, filtered, and concentrated to dryness under reduced pressure to afford the title compound (450 mg) as a yellow oil, which was used in the next step without further purification. LC-MS (ESI): mass calcd. for C₁₄H₁₈FN₃O₂ 279.14 m/z found 280.3 [M+H]⁺.

Step C. 2-(5-Fluoropyridin-2-yl)-6-isopropyl-6,7-dihydro-4H-pyrazolo[5,1-c][1,4]oxazine. H₃PO₄ (1.5 mL) was added into a solution consisting of 1-(3-(5-fluoropyridin-2-yl)-5-(hydroxymethyl)-1H-pyrazol-1-yl)-3-methylbutan-2-ol (330 mg, 1.18 mmol) and toluene (15 mL). The resultant suspension was stirred at 110° C. for 16 hours. The reaction mixture was gradually cooled to room-temperature, poured into H₂O (20 mL). The pH was adjusted to pH=8 with 3 M NaOH. The reaction mixture was extracted with ethyl acetate (30 mL×3). The combined organic extracts were washed with brine (15 mL), dried over anhydrous Na₂SO₄, filtered, and concentrated to dryness under reduced pressure to afford the title compound (300 mg), which was used in the next step without further purification. LC-MS (ESI): mass calcd. for C₁₄H₁₆FN₃O 261.13 m/z found 261.9 [M+H]⁺.

Step D. 3-Bromo-2-(5-fluoropyridin-2-yl)-6-isopropyl-6,7-dihydro-4H-pyrazolo[5,1-c][1,4]oxazine. The title compound was prepared in a manner analogous to Intermediate 37, Step B, except using 2-(5-fluoropyridin-2-yl)-6-isopropyl-6,7-dihydro-4H-pyrazolo[5,1-c][1,4]oxazine instead of 2-(5-fluoropyridin-2-yl)-6,7-dihydro-4H-pyrazolo[5,1-c][1,4]oxazine and 3-(5-fluoropyridin-2-yl)-6,7-dihydro-4H-pyrazolo[5,1-c][1,4]oxazine; and the reaction was run at 0° C. instead of room temperature. LC-MS (ESI): mass calcd. for C₁₄H₁₅BrFN₃O 339.04 m/z found 339.8 [M+H]⁺.

### Intermediate 79: 3-Bromo-2-(4-fluorophenyl)-6-(trifluoromethyl)-6,7-dihydro-4H-pyrazolo[5,1-c][1,4]oxazine

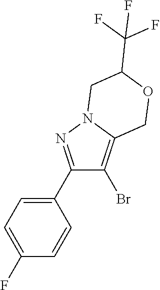 [SMILES: Fc1ccc(-c2nn3c(c2Br)COC(C(F)(F)F)C3)cc1]

Step A. 3-(4-Fluorophenyl)-1-(3,3,3-trifluoro-2-hydroxypropyl)-1H-pyrazole-5-carboxylic acid. A mixture of ethyl 3-(4-fluorophenyl)-1H-pyrazole-5-carboxylate (Intermediate 33, 5.0 g, 21 mmol), 3-bromo-1,1,1-trifluoropropan-2-ol (5.4 g, 28 mmol) and Cs₂CO₃ (13.9 g, 42.7 mmol) were stirred in MeCN (100 mL) for 16 hours at r.t, then 100 mL water was added to the reaction mixture at it was stirred at 75° C. for 3 hours. The mixture was extracted with ethyl acetate, the organic layer was dried (Na₂SO₄), filtered, and concentrated under reduced pressure. The resulting residue was purified by reverse-phase HPLC (Boston Uni C18 40*150*5 um column (eluent: 42% to 72% (v/v) CH₃CN and H₂O with 0.225% HCOOH)) to afford the title compound (4.5 g, 14 mmol, 66%). MS (ESI): mass calcd. for C₁₃H₁₀F₄N₂O₃ 318.1; found, 318.9 [M+H]⁺.

Step B. 1,1,1-Trifluoro-3-(3-(4-fluorophenyl)-5-(hydroxymethyl)-1H-pyrazol-1-yl)propan-2-ol. LiAlH₄ (268 mg, 7.06 mmol) was added in portions to a 0° C. solution consisting of 3-(4-fluorophenyl)-1-(3,3,3-trifluoro-2-hydroxypropyl)-1H-pyrazole-5-carboxylic acid (750 mg, 2.36 mmol) and THF (20 mL). The resultant mixture was stirred for 1 hours. The reaction mixture was gradually warmed to room-temperature. The mixture was quenched with water (0.3 mL) and then 15% NaOH. aq (0.3 mL), then Mg₂SO₄ (300 mg) was added to the mixture. The resultant mixture was filtered, the filter cake was washed with ethyl acetate (10 mL×3). The filtrate was concentrated to dryness under reduced pressure. The resulting residue was purified by FCC (eluent: petroleum ether:ethyl acetate=1:0 to 1:1) to afford the title compound (470 mg, 59%) as a white solid, which was used directly in the next step.

Step C. 2-(4-Fluorophenyl)-6-(trifluoromethyl)-6,7-dihydro-4H-pyrazolo[5,1-c][1,4]oxazine. t-BuOK (170 mg, 1.52 mmol) was added to a solution consisting of 1,1,1-trifluoro-3-(3-(4-fluorophenyl)-5-(hydroxymethyl)-1H-pyrazol-1-yl)propan-2-ol (470 mg, 1.55 mmol) and THF (10 mL). The resultant mixture was stirred at room-temperature for 15 mins. MsCl (540 mg, 4.71 mmol) and additional t-BuOK (350 mg, 3.12 mmol) were added to the mixture at room-temperature. The resultant mixture was stirred at room-temperature for 1 hour, then the heated at 75° C. for 2 hours. The reaction was quenched with sat. NaHCO₃(10 mL) and then stirred at room-temperature for 0.5 hours. The mixture was extracted with ethyl acetate (30 ml×3) and the combined organic extracts were concentrated to dryness under reduced pressure. The resulting residue was purified by FCC (eluent: petroleum ether:ethyl acetate=1:0 to 4:1) to afford the title compound (230 mg, 50%) as a white solid. MS (ESI): mass calcd. for C₁₃H₁₀F₄N₂O 286.1; m/z found 286.9 [M+H]⁺.

Step D. 3-Bromo-2-(4-fluorophenyl)-6-(trifluoromethyl)-6,7-dihydro-4H-pyrazolo[5,1-c][1,4]oxazine. NBS (170 mg, 0.955 mmol) was added to a mixture consisting of 2-(4-fluorophenyl)-6-(trifluoromethyl)-6,7-dihydro-4H-pyrazolo[5,1-c][1,4]oxazine (230 mg, 0.804 mmol) and dichloromethane (10 mL). The reaction mixture was stirred at room-temperature for 1 hour. The mixture was diluted with dichloromethane (15 mL) and washed with water (10 mL×3). The organic phase was concentrated to dryness under reduced pressure to give the residue, the residue was purified by FCC (eluent: petroleum ether:ethyl acetate=1:0 to 5:1) to afford the title compound (270 mg, 92%) as a white solid. MS (ESI): mass calcd. for C₁₃H₉BrF₄N₂O 363.98; m/z found 364.9 [M+H]⁺.

### Intermediate 80: 3-Bromo-2-(5-fluoropyridin-2-yl)-6-(trifluoromethyl)-6,7-dihydro-4H-pyrazolo[5,1-c][1,4]oxazine

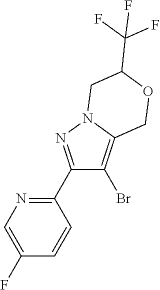 [SMILES: Fc1ccc(-c2nn3c(c2Br)COC(C(F)(F)F)C3)nc1]

Step A. Ethyl 3-(5-fluoropyridin-2-yl)-1-(3,3,3-trifluoro-2-hydroxypropyl)-1H-pyrazole-5-carboxylate. To a suspension of ethyl 3-(5-fluoropyridin-2-yl)-1H-pyrazole-5-carboxylate (Intermediate 34, 300 mg, 1.28 mmol) and Cs₂CO₃ (623 mg, 1.9 mmol) in DMF (6.4 mL) was added 3-bromo-1,1,1-trifluoro-2-propanol (0.27 mL, 2.6 mmol). The mixture was stirred at room temperature for 16 h then diluted with EtOAc (50 mL) and filtered. The filtrate was washed with 5% aqueous LiCl (2×25 mL) and brine (25 mL) then dried (Na₂SO₄) and filtered. Purification by chromatography (silica gel, 0-60% EtOAc/hexanes) afforded 284 mg (64%) of the title compound. MS (ESI): mass calcd. for C₁₄H₁₃F₄N₃O₃, 347.1; m/z found, 348.2 [M+H]⁺. ¹H NMR (400 MHz, CDCl₃): δ 8.48 (d, J=2.88 Hz, 1H), 7.94 (dd, J=8.76, 4.38 Hz, 1H), 7.52-7.43 (m, 2H), 5.07-4.97 (m, 1H), 4.94-4.86 (m, 1H), 4.57-4.47 (m, 1H), 4.40 (q, J=7.13 Hz, 2H), 4.25 (d, J=7.13 Hz, 1H), 1.41 (t, J=7.13 Hz, 3H).

Step B. 1,1,1-Trifluoro-3-(3-(5-fluoropyridin-2-yl)-5-(hydroxymethyl)-1H-pyrazol-1-yl)propan-2-ol. A solution of ethyl 3-(5-fluoropyridin-2-yl)-1-(3,3,3-trifluoro-2-hydroxypropyl)-1H-pyrazole-5-carboxylate (50 mg, 0.14 mmol) in THF (1.5 mL) was cooled to −78° C. then diisobutylaluminum hydride (0.32 mL of 1 M in toluene, 0.32 mmol) was added dropwise. The reaction was stirred at −78° C. for an hour then an additional portion of diisobutylaluminum hydride (0.32 mL of 1 M in toluene, 0.32 mmol) was added. The reaction was allowed to warm to room temperature then was stirred for 4 hours. The reaction was quenched with acetone then 30% aqueous potassium sodium tartrate (10 mL) was added and the mixture extracted with EtOAc (3×10 mL). The combined organics were dried (Na₂SO₄) and filtered to afford 46 mg (105%) of the title compound which was used without further purification. MS (ESI): mass calcd. for C₁₂H₁₁F₄N₃O₂, 305.1; m/z found, 306.2 [M+H]⁺. ¹H NMR (400 MHz, DMSO-d₆) δ 8.56 (d, J=3.00 Hz, 1H), 7.97 (dd, J=9.07, 4.32 Hz, 1H), 7.75 (td, J=8.79, 2.94 Hz, 1H), 6.72 (s, 1H), 5.46-5.40 (m, 1H), 4.60 (dd, J=6.75, 5.38 Hz, 2H), 4.41-4.30 (m, 3H).

Step C. 2-(5-Fluoropyridin-2-yl)-6-(trifluoromethyl)-6,7-dihydro-4H-pyrazolo[5,1-c][1,4]oxazine. A solution of 1,1,1-trifluoro-3-(3-(5-fluoropyridin-2-yl)-5-(hydroxymethyl)-1H-pyrazol-1-yl)propan-2-ol (203 mg, 0.67 mmol) in H₂SO₄ (0.7 mL, 13 mmol) was heated to 90° C. for 16 h. The reaction mixture was cooled then quenched with aqueous NaOH and extracted with EtOAc (2×10 mL). The combined organics were dried (Na₂SO₄) and filtered to afford 160 mg (84%) of the title compound. ¹H NMR (400 MHz, CDCl₃): δ 8.48 (d, J=3.00 Hz, 1H), 7.93 (dd, J=8.82, 4.44 Hz, 1H), 7.45 (td, J=8.44, 2.88 Hz, 1H), 6.65 (s, 1H), 5.19 (d, J=14.88 Hz, 1H), 4.94 (d, J=14.88 Hz, 1H), 4.49-4.27 (m, 3H).

Step D. 3-Bromo-2-(5-fluoropyridin-2-yl)-6-(trifluoromethyl)-6,7-dihydro-4H-pyrazolo[5,1-c][1,4]oxazine. The title compound was prepared in a manner analogous to Intermediate 37, Step B except using 2-(5-fluoropyridin-2-yl)-6-(trifluoromethyl)-6,7-dihydro-4H-pyrazolo[5,1-c][1,4]oxazine instead of 2-(5-fluoropyridin-2-yl)-6,7-dihydro-4H-pyrazolo[5,1-c][1,4]oxazine (Intermediate 37, Step A). MS (ESI): mass calcd. for C₁₂H₈BrF₄N₃O, 365.0; m/z found, 366.0 [M+H]⁺. ¹H NMR (400 MHz, CDCl₃): δ 8.59 (d, J=2.88 Hz, 1H), 7.99 (dd, J=8.76, 4.38 Hz, 1H), 7.53-7.46 (m, 1H), 5.12 (d, J=15.38 Hz, 1H), 4.84 (d, J=15.26 Hz, 1H), 4.48-4.35 (m, 2H), 4.34-4.24 (m, 1H).

### Intermediate 81: 3-Bromo-6-(fluoromethyl)-2-(4-fluorophenyl)-6,7-dihydro-4H-pyrazolo[5,1-c][1,4]oxazine

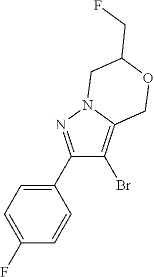 [SMILES: FCC1Cn2nc(-c3ccc(F)cc3)c(Br)c2CO1]

Step A. 1-(3-Fluoro-2-hydroxypropyl)-3-(4-fluorophenyl)-1H-pyrazole-5-carboxylic acid. To a suspension of ethyl 3-(4-fluorophenyl)-1H-pyrazole-5-carboxylate (Intermediate 33, 2.0 g, 8.5 mmol) and Cs₂CO₃ (8.3 g, 25.6 mmol) in DMF (11 mL) was added 1-chloro-3-fluoroisopropanol (0.727 mL, 10.2 mmol). The mixture was stirred at room temperature for 60 h then diluted with 1N HCl (38.4 mL) to adjust pH=2. The resulting mixture was extracted with DCM-IPA (4/1, 3×25 mL) then dried (Na₂SO₄), filtered and evaporated under reduced pressure to afford 3 g (99.6%, 80% pure) of the title compound. MS (ESI): mass calcd. for C₁₃H₁₂F₂N₂O₃, 282.1; m/z found, 283.1 [M+H]⁺.

Step B. 1-Fluoro-3-(3-(4-fluorophenyl)-5-(hydroxymethyl)-1H-pyrazol-1-yl)propan-2-ol. To a solution of 1-(3-fluoro-2-hydroxypropyl)-3-(4-fluorophenyl)-1H-pyrazole-5-carboxylic acid (2.8 g, 80% pure, 7.9 mmol) in THF (118 mL) was added borane tetrahydrofuran complex (32 mL of 1 M in THF, 32 mmol) dropwise and the resulting solution was stirred at 50° C. for 18 hours. Then it was cooled down and an additional portion of borane tetrahydrofuran complex (16 mL of 1 M in THF, 16 mmol) was added and the resulting solution was further stirred at 50° C. for 4 hours. The reaction was quenched with MeOH (4.4 mL) then concentrated under reduced pressure to remove the solvents. The resulting residue was purified by chromatography (silica gel, 0-50% EtOAc/DCM) to afford 1.38 g (65%) of the title compound. MS (ESI): mass calcd. for C₁₃H₁₄F₂N₂O₂, 268.1; m/z found, 269.1 [M+H]⁺.

Step C. 6-(Fluoromethyl)-2-(4-fluorophenyl)-6,7-dihydro-4H-pyrazolo[5,1-c][1,4]oxazine. A solution of 1-fluoro-3-(3-(4-fluorophenyl)-5-(hydroxymethyl)-1H-pyrazol-1-yl)propan-2-ol (1.38 g, 5.1 mmol) in H₂SO₄ (11.8 mL, 217.6 mmol) was heated to 120° C. for 18 h. The reaction mixture was cooled then quenched with aqueous NaOH and extracted with EtOAc (3×50 mL). The combined organics were dried (Na₂SO₄), filtered and evaporated. The resulting residue was purified by chromatography (silica gel, 0-50% EtOAc/Hexane) to afford 810 mg (63%) of the title compound. MS (ESI): mass calcd. for C₁₃H₁₂F₂N₂O, 250.1; m/z found, 251.1 [M+H]⁺.

Step D. 3-Bromo-6-(fluoromethyl)-2-(4-fluorophenyl)-6,7-dihydro-4H-pyrazolo[5,1-c][1,4]oxazine. To a solution of 6-(fluoromethyl)-2-(4-fluorophenyl)-6,7-dihydro-4H-pyrazolo[5,1-c][1,4]oxazine (810 mg, 3.2 mmol) in DMF (3.2 mL) was added N-bromosuccinimide (720 mg, 4.0 mmol). The reaction mixture was stirred at room temperature for 1 h and then evaporation removed the solvents. The resulting residue was purified by silica gel chromatography (0-40% EtOAc in hexanes) gave the title compound (680 mg, 64% yield). MS (ESI): mass calcd. for C₁₃H₁₁BrF₂N₂O, 328.0; m/z found, 329.1 [M+H]⁺.

### Intermediate 82: 3′-Bromo-2′-(4-fluorophenyl)-4′H,6′H-spiro[cyclopropane-1,7′-pyrazolo[5,1-c][1,4]oxazine]

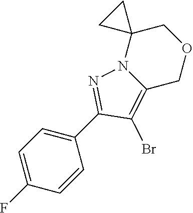 [SMILES: Fc1ccc(-c2nn3c(c2Br)COCC32CC2)cc1]

Step A: Ethyl 1-(1-(ethoxycarbonyl)cyclopropyl)-3-(4-fluorophenyl)-1H-pyrazole-5-carboxylate. Ethyl 2,4-dibromobutanoate (3.05 g, 11.1 mmol) was added to a solution consisting of ethyl 3-(4-fluorophenyl)-1H-pyrazole-5-carboxylate (Intermediate 33, 2.0 g, 8.5 mmol), Cs₂CO₃ (5.57 g, 17.1 mmol), and CH₃CN (30 mL). The resultant mixture was stirred at room-temperature for 20 hours. The reaction mixture was poured into H₂O (40 mL) and extracted with ethyl acetate (30 mL×3). The combined organic extracts were dried over anhydrous Na₂SO₄, filtered, and concentrated to dryness under reduced pressure. The resulting residue was purified by FCC (SiO₂, eluent: petroleum ether:ethyl acetate=1:0 to 3:1) to afford the title compound (700 mg, 19%) as a yellow solid.

Step B: (3-(4-Fluorophenyl)-1-(1-(hydroxymethyl)cyclopropyl)-1H-pyrazol-5-yl)methanol. LiAlH₄ (230 mg, 6.06 mmol) was added in portions to a 0° C. solution consisting of ethyl 1-(1-(ethoxycarbonyl)cyclopropyl)-3-(4-fluorophenyl)-1H-pyrazole-5-carboxylate (700 mg, 2.02 mmol) and THF (10 mL) at a 100 mL three-necked round-bottomed flask under N₂. The resultant mixture was stirred for 3 hours The reaction mixture was gradually warmed to room-temperature. H₂O (0.23 mL) was added to the reaction mixture at 0° C., and then 15% aq. NaOH (0.23 mL) was added to the mixture. The resultant mixture was filtered over Celite® pad and the filter cake was washed with MeOH (5 mL×4). The filtrate was dried over anhydrous Na₂SO₄, filtered, and concentrated to dryness under reduced pressure to give the title compound (700 mg) as a white solid, which was used in the next step without further purification. LC-MS (ESI): mass calcd. for C₁₄H₁₅FN₂O₂ 262.11 m/z found 262.9 [M+H]⁺.

Step C: 2′-(4-Fluorophenyl)-4′H,6′H-spiro[cyclopropane-1,7′-pyrazolo[5,1-c][1,4]oxazine]. t-BuOK (200 mg, 1.78 mmol) was added to a solution consisting of (3-(4-fluorophenyl)-1-(1-(hydroxymethyl)cyclopropyl)-1H-pyrazol-5-yl)methanol (550 mg, 2.10 mmol) and THF (10 mL). The resultant mixture was stirred at room-temperature for 15 minutes and then treated with MsCl (530 mg, 4.63 mmol) and additional t-BuOK (505 mg, 4.50 mmol) at room-temperature. The resultant mixture was stirred at room-temperature for another 1 hour and at 75° C. for 2 hours. The reaction mixture was quenched with sat NaHCO₃(20 mL). The mixture was stirred at room-temperature for 0.5 hours and extracted with ethyl acetate (20 mL×2). The combined organic extracts were concentrated to dryness under reduced pressure to afford the title compound (500 mg), which was used in the next step without further purification. LC-MS (ESI): mass calcd. for C₁₄H₁₃FN₂O 244.10 m/z found 245.0 [M+H]⁺.

Step D: 3′-Bromo-2′-(4-fluorophenyl)-4′H,6′H-spiro[cyclopropane-1,7′-pyrazolo[5,1-c][1,4]oxazine]. The title compound was prepared in a manner analogous to Intermediate 37, Step B except using 2′-(4-fluorophenyl)-4′H,6′H-spiro[cyclopropane-1,7′-pyrazolo[5,1-c][1,4]oxazine] instead of 2-(5-fluoropyridin-2-yl)-6,7-dihydro-4H-pyrazolo[5,1-c][1,4]oxazine and 3-(5-fluoropyridin-2-yl)-6,7-dihydro-4H-pyrazolo[5,1-c][1,4]oxazine (Intermediate 37, Step A) and DCM instead of DMF. LC-MS (ESI): mass calcd. for C₁₄H₁₂BrFN₂O 322.01 m/z found 322.7 [M+H]⁺.

### Intermediate 83: 3-Bromo-6-cyclopropyl-2-(5-fluoropyridin-2-yl)-6,7-dihydro-4H-pyrazolo[5,1-c][1,4]oxazine

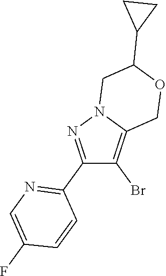 [SMILES: Fc1ccc(-c2nn3c(c2Br)COC(C2CC2)C3)nc1]

Step A. 1-Cyclopropyl-2-(3-(5-fluoropyridin-2-yl)-5-(hydroxymethyl)-1H-pyrazol-1-yl)ethanone. 2-Bromo-1-cyclopropylethanone (405.0 mg, 2.485 mmol) was added to a solution consisting of (3-(5-fluoropyridin-2-yl)-1H-pyrazol-5-yl)methanol (Intermediate 35, product from Step A, 400.0 mg, 2.071 mmol), Cs₂CO₃ (1.349 g, 4.141 mmol), and CH₃CN (20 mL). The resultant mixture was stirred at room-temperature for 2 hours. The suspension was filtered and the filtrate was concentrated to dryness under reduced pressure. The resulting residue was purified by FCC (SiO₂, eluent: petroleum ether:ethyl acetate=2:1 to 1:3) to afford the title compound (350 mg, 60%) as a yellow solid. LC-MS (ESI): mass calcd. for C₁₄H₁₄FN₃O₂ 275.11 m/z, found 276.1 [M+H]⁺.

Step B. 1-Cyclopropyl-2-(3-(5-fluoropyridin-2-yl)-5-(hydroxymethyl)-1H-pyrazol-1-yl)ethanol. Sodium tetrahydroborate (96.20 mg, 2.543 mmol) was added to a solution consisting of 1-cyclopropyl-2-(3-(5-fluoropyridin-2-yl)-5-(hydroxymethyl)-1H-pyrazol-1-yl)ethanone (350.0 mg, 1.271 mmol) and MeOH (10 mL). The resultant mixture was stirred at room-temperature for 2 hours. The reaction mixture was quenched with sat. NH₄Cl (50 mL) and extracted with ethyl acetate (50 mL×2). The combined organic extracts were washed with brine (50 mL), dried over anhydrous Na₂SO₄, filtered, and concentrated to dryness under reduced pressure to give the title compound (350 mg, 97%) as a yellow solid. LC-MS (ESI): mass calcd. for C₁₄H₁₆FN₃O₂ 277.12 m/z, found 278.1 [M+H]⁺.

Step C. 6-Cyclopropyl-2-(5-fluoropyridin-2-yl)-6,7-dihydro-4H-pyrazolo[5,1-c][1,4]oxazine. Zinc(II) chloride (1.57 g, 11.5 mmol) was added to a solution consisting of 1-cyclopropyl-2-(3-(5-fluoropyridin-2-yl)-5-(hydroxymethyl)-1H-pyrazol-1-yl)ethanol (320 mg, 1.15 mmol), and 1,2-dichloroethane (10 mL). The resultant mixture was stirred at 90° C. for 12 hours. The reaction mixture was quenched with water (50 mL) and extracted with ethyl acetate (50 mL×2). The combined organic extracts were dried over anhydrous Na₂SO₄, filtered, and concentrated to dryness under reduced pressure. The resulting residue was purified by FCC (SiO₂, eluent: petroleum ether:ethyl acetate=2:1 to 0:1) to afford the title compound (150 mg, 40%) as a yellow solid. LC-MS (ESI): mass calcd. for C₁₄H₁₄FN₃O 259.11 m/z, found 260.1 [M+H]⁺.

Step D. 3-Bromo-6-cyclopropyl-2-(5-fluoropyridin-2-yl)-6,7-dihydro-4H-pyrazolo[5,1-c][1,4]oxazine. The title compound was prepared in a manner analogous to Intermediate 37, Step B, except using 6-cyclopropyl-2-(5-fluoropyridin-2-yl)-6,7-dihydro-4H-pyrazolo[5,1-c][1,4]oxazine instead of 2-(5-fluoropyridin-2-yl)-6,7-dihydro-4H-pyrazolo[5,1-c][1,4]oxazine and 3-(5-fluoropyridin-2-yl)-6,7-dihydro-4H-pyrazolo[5,1-c][1,4]oxazine, in DCM instead of DMF. ¹H NMR (400 MHz, CDCl₃): δ 8.57 (d, J=2.8 Hz, 1H), 7.98 (dd, J=4.4, 8.8 Hz, 1H), 7.47 (dt, J=2.8, 8.4 Hz, 1H), 4.97 (d, J=15.6 Hz, 1H), 4.66 (d, J=15.2 Hz, 1H), 4.30 (dd, J=3.2, 12.8 Hz, 1H), 4.08 (dd, J=10.4, 12.4 Hz, 1H), 3.30-3.17 (m, 1H), 1.15-1.03 (m, 1H), 0.78-0.63 (m, 2H), 0.59-0.49 (m, 1H), 0.45-0.34 (m, 1H).

### Intermediate 84: 3-Bromo-6-cyclobutyl-2-(5-fluoropyridin-2-yl)-6,7-dihydro-4H-pyrazolo[5,1-c][1,4]oxazine

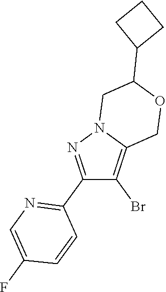 [SMILES: Fc1ccc(-c2nn3c(c2Br)COC(C2CCC2)C3)nc1]

Step A. 1-Cyclobutyl-2-(3-(5-fluoropyridin-2-yl)-5-(hydroxymethyl)-1H-pyrazol-1-yl)ethanone. 2-Bromo-1-cyclobutylethanone (549.8 mg, 3.106 mmol) was added to a mixture consisting of (3-(5-fluoropyridin-2-yl)-1H-pyrazol-5-yl)methanol (Intermediate 35, product from Step A, 500.0 mg, 2.588 mmol), Cs₂CO₃ (1.687 g, 5.177 mmol), and CH₃CN (20 mL). The resultant mixture was stirred at room-temperature for 12 hours. The resultant yellow suspension was filtered and the filtrate concentrated to dryness under reduced pressure. The resulting residue was purified by FCC (SiO₂, eluent: petroleum ether:ethyl acetate=3:1 to 1:2) to afford the title compound (360 mg, 44%) as yellow oil. LC-MS (ESI): mass calcd. for C₁₅H₁₆FN₃O₂ 289.12 m/z, found 290.1 [M+H]⁺. ¹H NMR (400 MHz, DMSO-d₆) δ 8.56 (d, J=2.8 Hz, 1H), 7.91 (dd, J=4.4, 8.8 Hz, 1H), 7.76-7.70 (m, 1H), 6.73 (s, 1H), 5.14 (s, 2H), 4.44 (d, J=5.6 Hz, 2H), 3.48-3.37 (m, 1H), 2.22-2.13 (m, 1H), 2.11-2.03 (m, 2H), 1.96-1.85 (m, 2H), 1.78-1.69 (m, 1H).

Step B. 1-Cyclobutyl-2-(3-(5-fluoropyridin-2-yl)-5-(hydroxymethyl)-1H-pyrazol-1-yl)ethanol. Sodium tetrahydroborate (94.16 mg, 2.489 mmol) was added to a solution consisting of 1-cyclobutyl-2-(3-(5-fluoropyridin-2-yl)-5-(hydroxymethyl)-1H-pyrazol-1-yl)ethanone (360.0 mg, 1.244 mmol) and MeOH (10 mL). The resultant mixture was stirred at room-temperature for 2 hours. The reaction mixture was quenched with saturated NH₄Cl (30 mL) and extracted with ethyl acetate (30 mL×3). The combined organic extracts were washed with brine (50 mL), dried over anhydrous Na₂SO₄, filtered, and concentrated to dryness under reduced pressure to afford the title compound (350 mg, 91%) as a yellow oil. LC-MS (ESI): mass calcd. for C₁₅H₁₈FN₃O₂ 291.14 m/z, found 292.2 [M+H]⁺. ¹H NMR (400 MHz, DMSO-d₆) δ 8.54 (d, J=2.8 Hz, 1H), 7.94 (dd, J=4.8, 8.8 Hz, 1H), 7.73 (dt, J=2.8, 8.8 Hz, 1H), 6.67 (s, 1H), 5.32 (t, J=5.6 Hz, 1H), 5.03 (d, J=5.6 Hz, 1H), 4.56 (t, J=5.2 Hz, 1H), 4.00-3.97 (m, 1H), 3.83-3.75 (m, 1H), 2.37-2.28 (m, 1H), 1.92-1.70 (m, 6H). ¹⁹F NMR (376 MHz, DMSO-d₆) δ −129.55 (s, 1F).

Step C. 6-Cyclobutyl-2-(5-fluoropyridin-2-yl)-6,7-dihydro-4H-pyrazolo[5,1-c][1,4]oxazine. KOH (191 mg, 3.40 mmol) and H₂O (1 mL) were added to a solution consisting of 1-cyclobutyl-2-(3-(5-fluoropyridin-2-yl)-5-(hydroxymethyl)-1H-pyrazol-1-yl)ethanol (330 mg, 1.13 mmol), TsCl (238 mg, 1.25 mmol), and 1,4-dioxane (10 mL). The resultant mixture was stirred at 100° C. for 12 hours. The reaction mixture was concentrated to dryness under reduced pressure. The resulting residue was purified by FCC (SiO₂, eluent: petroleum ether:ethyl acetate=10:1 to 1:1) to afford the title compound (150 mg, 48%) as a white solid. ¹H NMR (400 MHz, CDCl₃): δ 8.46 (d, J=2.8 Hz, 1H), 7.90 (dd, J=4.4, 8.8 Hz, 1H), 7.42 (dt, J=2.8, 8.4 Hz, 1H), 6.57 (s, 1H), 5.03 (d, J=14.8 Hz, 1H), 4.79 (d, J=15.2 Hz, 1H), 4.17 (d, J=9.6 Hz, 1H), 3.86-3.79 (m, 2H), 2.65-2.54 (m, 1H), 2.14-1.90 (m, 6H).

Step D. 3-Bromo-6-cyclobutyl-2-(5-fluoropyridin-2-yl)-6,7-dihydro-4H-pyrazolo[5,1-c][1,4]oxazine. The title compound was prepared in a manner analogous to Intermediate 37, Step B, except using 6-cyclobutyl-2-(5-fluoropyridin-2-yl)-6,7-dihydro-4H-pyrazolo[5,1-c][1,4]oxazine instead of 2-(5-fluoropyridin-2-yl)-6,7-dihydro-4H-pyrazolo[5,1-c][1,4]oxazine and 3-(5-fluoropyridin-2-yl)-6,7-dihydro-4H-pyrazolo[5,1-c][1,4]oxazine, in DCM instead of DMF. ¹H NMR (400 MHz, CDCl₃): δ 8.57 (d, J=2.8 Hz, 1H), 7.98 (dd, J=4.4, 8.8 Hz, 1H), 7.47 (dt, J=2.8, 8.4 Hz, 1H), 4.96 (d, J=15.2 Hz, 1H), 4.69 (d, J=15.2 Hz, 1H), 4.15 (d, J=9.2 Hz, 1H), 3.84-3.77 (m, 2H), 2.64-2.54 (m, 1H), 2.13-1.91 (m, 6H).

### Intermediate 85: 3-Bromo-2-(5-fluoropyridin-2-yl)-4,5,6,7-tetrahydro-4,7-ethanopyrazolo[1,5-a]pyridine

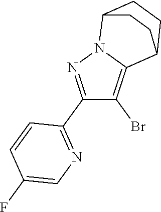 [SMILES: Fc1ccc(-c2nn3c(c2Br)C2CCC3CC2)nc1]

Step A: 1-(5-Fluoropyridin-2-yl)ethan-1-one. Methylmagnesium bromide (1M in THF, 18 mL) was added dropwise to a −60° C. solution consisting of 5-fluoropicolinonitrile (2.0 g, 16.4 mmol) and anhydrous THF (50 mL) under N₂. The reaction was stirred at −60° C. for 1-2 hours, and then at room-temperature for 4 hours. The reaction was quenched with sat·NH₄Cl solution, extracted with EtOAc (50 mL×3), washed with brine (30 mL), dried on anhydrous Na₂SO₄ and concentrated to dryness. The resulting residue was purified (FCC, SiO₂) to afford the title compound (1.5 g, 68%) as a yellow oil.

Step B: Methyl 3-(5-fluoropyridin-2-yl)-3-oxopropanoate. Sodium hydride in mineral oil (1.73 g, 60% purity, 43.3 mmol) was added in portions to a 0° C. (ice/water) solution consisting of 1-(5-fluoropyridin-2-yl)ethan-1-one (3.00 g, 21.6 mmol) and dimethyl carbonate (72.7 mL). The resulting mixture was stirred at room temperature for 3 h to give a orange/red suspension. The resultant mixture was poured into water (100 mL) and extracted with ethyl acetate (100 mL×2). The combined organic extracts were washed with brine (100 mL), dried over anhydrous Na₂SO₄, filtered, and concentrated to dryness under reduced pressure. The resulting reside was purified (FCC, SiO₂) to afford the title compound (3.4 g, 57% yield) as yellow oil. MS (ESI): mass calcd. for C₉H₈FNO₃ 197.2; m/z found 198.2 [M+H]⁺.

Step C: (4s,7s)-3-Bromo-2-(5-fluoropyridin-2-yl)-4,5,6,7-tetrahydro-4,7-ethanopyrazolo[1,5-a]pyridine. The title compound was prepared in a manner analogous to Intermediate 143, Steps A-E, except using methyl 4-oxocyclohexanecarboxylate instead of methyl 3-oxocyclopentanecarboxylate in Step A and methyl 3-(5-fluoropyridin-2-yl)-3-oxopropanoate instead of ethyl 3-(4-fluorophenyl)-3-oxopropanoate in Step E. MS (ESI): mass calcd. for C₁₄H₁₃BrFN₃ 321.0; m/z found 322.0 [M+H]⁺.

### Intermediate 86: 3-Bromo-2-(5-fluoropyridin-2-yl)-6,6-bis(methyl-d₃)-6,7-dihydro-4H-pyrazolo[5,1-c][1,4]oxazine

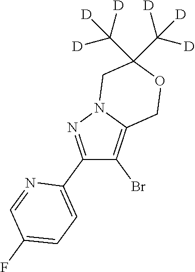 [SMILES: [2H]C([2H])([2H])C1(C([2H])([2H])[2H])Cn2nc(-c3ccc(F)cn3)c(Br)c2CO1]

Step A: Methyl 2-(benzyloxy)acetate. To a solution of phenylmethanol (10 g, 92 mmol) in THF under nitrogen was added NaH (60 percent) (7.00 g, 183 mmol) at 0° C. and the reaction mixture was allowed to stir at 0° C. for 0.5 hrs. Then, methyl 2-bromoacetate (17.0 mL, 184 mmol) was added at 0° C. and the reaction mixture was allowed to warm to RT and further stirred for 16 h. The reaction mixture was quenched with ice-water and concentrated. The residue was re-dissolved in EtOAc and washed with brine, dried over anhydrous sodium sulfate and concentrated. It was further purified by flash column to obtain the title compound as colorless oil (16 g, 96%).

Step B: 2-((Benzyloxy)methyl)propan-1,1,1,3,3,3-d₆-2-ol. methyl-d₃-magnesium-iodide (100 mL, 100 mmol, 1 M in ethoxyethane) was added dropwise to a 0° C. (ice/water) mixture consisting of methyl 2-(benzyloxy)acetate (4.5 g, 25 mmol) and THF (50 mL). The resulting mixture was stirred for 16 hours. The reaction mixture was gradually warmed to room-temperature. The mixture was quenched with sat. aq. NH₄Cl (100 mL) and extracted with ethyl acetate (60 mL×3). The combined organic extracts were dried over anhydrous Na₂SO₄, filtered and concentrated to dryness under reduced pressure to afford the crude compound, which was purified by flash column to afford the title compound (3.6 g, 77%), as an oil.

Step C: 2-(Methyl-d₃)propane-3,3,3-d₃-1,2-diol. 2-((Benzyloxy)methyl)propan-1,1,1,3,3,3-d₆-2-ol (3.6 g, 19 mmol), Pd/C (1.5 g), and EA (30 mL) were added to a 75 mL hydrogenation bottle. The resultant mixture was stirred under H₂ (50 psi) at 50° C. for 16 hours. The suspension was filtered through a pad of Celite® and the pad washed with EA (60 mL). The filtrate was concentrated to dryness under reduced pressure to afford the title product (1.5 g, crude) as a yellow oil, which was used in the next step without further purification. ¹H NMR (400 MHz, CDCl₃) δ 3.53-3.34 (m, 2H), 2.48-2.13 (m, 2H).

Step D: 2-Hydroxy-2-(methyl-d₃)propyl-3,3,3-d₃ 4-methylbenzenesulfonate. TosCl (2.97 g, 15.6 mmol) was added to a solution consisting of 2-(methyl-d₃)propane-3,3,3-d₃-1,2-diol (1.0 g, 10 mmol), TEA (3.62 ml, 26.0 mmol), DMAP (127 mg, 1.04 mmol), and dichloromethane (20 mL). The resulting mixture was stirred at room-temperature for 16 hours. The reaction mixture concentrated to dryness under reduced pressure. The resulting residue was purified by flash column to afford the title compound (1.8 g, 69%) as yellow oil.

Step E: 2-((3-(5-Fluoropyridin-2-yl)-5-(hydroxymethyl)-1H-pyrazol-1-yl)methyl)propan-1,1,1,3,3,3-d₆-2-ol. 2-Hydroxy-2-(methyl-d₃)propyl-3,3,3-d₃ 4-methylbenzenesulfonate (500 mg, 1.997 mmol), 2-(5-(((tert-butyldimethylsilyl)oxy)methyl)-1H-pyrazol-3-yl)-5-fluoropyridine (Intermediate 35, 614.03 mg, 1.997 mmol), Cs₂CO₃ (3.25 g, 9.975 mmol), KI (331.6 mg, 1.998 mmol), and DMA (12 mL) were added to a 20 mL microwave tube. The resulting mixture was heated at 120° C. via microwave irradiation for 0.5 hours. The reaction mixture was cooled to room-temperature. The mixture was poured into water (50 mL) and extracted with ethyl acetate (50 mL×3). The combined organic layers were washed with brine (50 mL), dried over anhydrous Na₂SO₄, filtered and concentrated under reduced pressure. The resulting residue was purified by FCC (SiO₂) to afford the compound (280 mg, 37.67%) as a clean oil. MS (ESI): mass calcd. for C₁₃H₁₀D₆FN₃O₂ 271.3; m/z found 272.2 [M+H]⁺.

Step F: 2-(5-Fluoropyridin-2-yl)-6,6-bis(methyl-d₃)-6,7-dihydro-4H-pyrazolo[5,1-c][1,4]oxazine. KOH (380.5 mg, 6.782 mmol) and H₂O (2.5 mL) were added to a solution consisting of 2-((3-(5-fluoropyridin-2-yl)-5-(hydroxymethyl)-1H-pyrazol-1-yl)methyl)propan-1,1,1,3,3,3-d₆-2-ol (460 mg, 1.695 mmol), TsCl (517.2 mg, 2.713 mmol) and dioxane (10 mL). The resulting mixture was stirred at 100° C. for 12 hours. The reaction mixture was concentrated to dryness under reduced pressure. The resulting residue was purified, (FCC, SiO₂) to afford the title compound (150 mg, 34.93% yield) as a white solid. ¹H NMR (400 MHz, DMSO-d₆) δ 8.56 (d, J=2.9 Hz, 1H), 7.97-7.92 (m, 1H), 7.78-7.72 (m, 1H), 6.59 (s, 1H), 4.84 (s, 2H), 4.00 (s, 2H).

Step G: 3-Bromo-2-(5-fluoropyridin-2-yl)-6,6-bis(methyl-d₃)-6,7-dihydro-4H-pyrazolo[5,1-c][1,4]oxazine. NBS (112 mg, 0.629 mmol) was added to a solution consisting of 2-(5-fluoropyridin-2-yl)-6,6-bis(methyl-d₃)-6,7-dihydro-4H-pyrazolo[5,1-c][1,4]oxazine (150 mg, 0.592 mmol) and dichloromethane (5 mL). The resulting mixture was stirred at room-temperature for 2 hours then quenched with water (50 mL) and extracted with ethyl acetate (50 mL×3). The combined organic extracts were dried over anhydrous Na₂SO₄, filtered, and concentrated to dryness under reduced pressure. The resulting residue was purified by flash column to afford the title compound (145 mg, 73.65%) as a white solid. ¹H NMR (400 MHz, DMSO-d₆) δ 8.64 (d, J=3.0 Hz, 1H), 7.95-7.90 (m, 1H), 7.84-7.77 (m, 1H), 4.75 (s, 2H), 4.02 (s, 2H).

### Intermediate 87: (Racemic) cis-3-Bromo-2-(4-fluorophenyl)-6,7-dimethyl-6,7-dihydro-4H-pyrazolo[5,1-c][1,4]oxazine

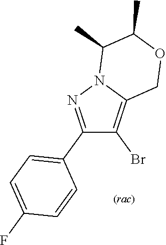 [SMILES: C[C@H]1OCc2c(Br)c(-c3ccc(F)cc3)nn2[C@H]1C]

Step A. Ethyl 3-(4-fluorophenyl)-1-(3-oxobutan-2-yl)-1H-pyrazole-5-carboxylate. 3-Bromobutan-2-one (2.3 mL, 3.8 mmol) was added to a solution consisting of ethyl 3-(4-fluorophenyl)-1H-pyrazole-5-carboxylate (Intermediate 33, 4.0 g, 17 mmol), Cs₂CO₃ (11 g, 34 mmol), and CH₃CN (60 mL). The resultant mixture was stirred at room-temperature for 2 hours. The suspension was filtered through a pad of Celite® and the pad washed with ethyl acetate (50 mL). The filtrate was concentrated to dryness under reduced pressure. The resulting residue was purified by FCC (SiO₂, eluent: petroleum ether:ethyl acetate=1:0 to 15:1) to afford the title compound (3.7 g, 71%) as a colorless oil. MS (ESI): mass calcd. for C₁₆H₁₇FN₂O₃ 304.1 m/z found 305.4 [M+H]⁺.

Step B. 3-(3-(4-Fluorophenyl)-5-(hydroxymethyl)-1H-pyrazol-1-yl)butan-2-ol. LiAlH₄ (1.4 g, 37 mmol) was added in portions to a 0° C. (ice/water) solution consisting of ethyl 3-(4-fluorophenyl)-1-(3-oxobutan-2-yl)-1H-pyrazole-5-carboxylate (3.7 g, 12 mmol) and THF (50 mL) under N₂. The resultant mixture was stirred for 2 hours. The reaction mixture was gradually warmed to room-temperature then diluted with THF (40 mL) and quenched with H₂O (1.4 mL) and aq. NaOH (15 wt %, 1.4 mL) slowly. The mixture was stirred at room-temperature for 0.5 hours then further treated with H₂O (4.2 mL). The resultant mixture was stirred at room-temperature for another 0.5 hours and then dried over anhydrous MgSO₄. The suspension was filtered through a pad of Celite® and the pad washed with ethyl acetate (100 mL). The filtrate was concentrated to dryness under reduced pressure to afford the title compound (3.0 g) as a yellow semi-solid, which was used in the next step without further purification. LC-MS (ESI): mass calcd. for C₁₄H₁₇FN₂O₂ 264.1 m/z found 265.1 [M+H]⁺.

Step C. (Racemic) cis-2-(4-Fluorophenyl)-6,7-dimethyl-6,7-dihydro-4H-pyrazolo[5,1-c][1,4]oxazine and (Racemic) trans-2-(4-fluorophenyl)-6,7-dimethyl-6,7-dihydro-4H-pyrazolo[5,1-c][1,4]oxazine. 3-(3-(4-Fluorophenyl)-5-(hydroxymethyl)-1H-pyrazol-1-yl)butan-2-ol (3.0 g) was added to a solution consisting of H₃PO₄ (3 mL) and toluene (30 mL). The resultant mixture was heated at 130° C. for 16 hours. The reaction mixture was cooled to room-temperature. The mixture was combined with additional batches then poured it into sat. NaHCO₃ (60 mL), and the resultant mixture extracted with ethyl acetate (70 mL×3). The combined organic extracts were washed with brine (60 mL), dried over anhydrous Na₂SO₄, filtered, and concentrated to dryness under reduced pressure. The resulting residue was purified by FCC (SiO₂, eluent: petroleum ether:ethyl acetate=10:1 to 4:1) to afford (Racemic) cis-2-(4-fluorophenyl)-6,7-dimethyl-6,7-dihydro-4H-pyrazolo[5,1-c][1,4]oxazine (800 mg, crude) as a yellow solid and (Racemic) trans-2-(4-fluorophenyl)-6,7-dimethyl-6,7-dihydro-4H-pyrazolo[5,1-c][1,4]oxazine (500 mg, 17%) as a white solid. (Racemic) cis-2-(4-Fluorophenyl)-6,7-dimethyl-6,7-dihydro-4H-pyrazolo[5,1-c][1,4]oxazine was further purified by preparative HPLC Method A to afford pure product. The product was suspended in water (10 mL), the mixture frozen using dry ice/EtOH, and then lyophilized to dryness to afford (Racemic) cis-2-(4-fluorophenyl)-6,7-dimethyl-6,7-dihydro-4H-pyrazolo[5,1-c][1,4]oxazine (520 mg) as a white solid. ¹H NMR (400 MHz, CDCl₃): δ 7.84-7.67 (m, 2H), 7.12-7.04 (m, 2H), 6.20 (s, 1H), 5.03-4.91 (m, 1H), 4.86-4.76 (m, 1H), 4.32-4.20 (m, 1H), 4.15-3.99 (m, 1H), 1.44 (d, J=6.7 Hz, 3H), 1.35 (d, J=6.4 Hz, 3H). (Racemic) trans-2-(4-Fluorophenyl)-6,7-dimethyl-6,7-dihydro-4H-pyrazolo[5,1-c][1,4]oxazine. MS (ESI): mass calcd. for C₁₄H₁₅FN₂O 246.12 m/z found 247.3 [M+H]⁺. ¹H NMR (400 MHz, CDCl₃): δ 7.83-7.71 (m, 2H), 7.14-7.03 (m, 2H), 6.24 (s, 1H), 5.01-4.92 (m, 1H), 4.84-4.74 (m, 1H), 4.04-3.91 (m, 1H), 3.71-3.60 (m, 1H), 1.63 (d, J=6.5 Hz, 3H), 1.42 (d, J=6.3 Hz, 3H).

Step D: (Racemic) cis-3-Bromo-2-(4-fluorophenyl)-6,7-dimethyl-6,7-dihydro-4H-pyrazolo[5,1-c][1,4]oxazine. The title compound was prepared in a manner analogous to Intermediate 37, Step B, except using cis-2-(4-fluorophenyl)-6,7-dimethyl-6,7-dihydro-4H-pyrazolo[5,1-c][1,4]oxazine instead of 2-(5-fluoropyridin-2-yl)-6,7-dihydro-4H-pyrazolo[5,1-c][1,4]oxazine and 3-(5-fluoropyridin-2-yl)-6,7-dihydro-4H-pyrazolo[5,1-c][1,4]oxazine. MS (ESI): mass calcd. for C₁₄H₁₄BrFN₂O 324.0 m/z found 324.8 [M+H]⁺.

### Intermediate 88: (Racemic) trans-3-Bromo-2-(4-fluorophenyl)-6,7-dimethyl-6,7-dihydro-4H-pyrazolo[5,1-c][1,4]oxazine

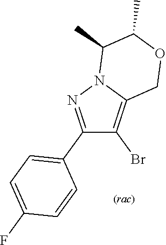 [SMILES: C[C@@H]1OCc2c(Br)c(-c3ccc(F)cc3)nn2[C@H]1C]

The title compound was prepared in a manner analogous to Intermediate 37, Step B, except using (Racemic) trans-2-(4-fluorophenyl)-6,7-dimethyl-6,7-dihydro-4H-pyrazolo[5,1-c][1,4]oxazine (Intermediate 87, product from Step C) instead of 2-(5-fluoropyridin-2-yl)-6,7-dihydro-4H-pyrazolo[5,1-c][1,4]oxazine and 3-(5-fluoropyridin-2-yl)-6,7-dihydro-4H-pyrazolo[5,1-c][1,4]oxazine. MS (ESI): mass calcd. for C₁₄H₁₄BrFN₂O 324.0 m/z found 325.0[M+H]⁺.

### Intermediate 89: (Racemic) cis-3-Bromo-2-(5-fluoropyridin-2-yl)-6,7-dimethyl-6,7-dihydro-4H-pyrazolo[5,1-c][1,4]oxazine

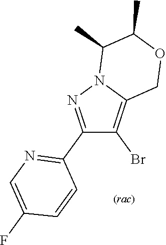 [SMILES: C[C@H]1OCc2c(Br)c(-c3ccc(F)cn3)nn2[C@H]1C]

Step A. 3-(3-(5-Fluoropyridin-2-yl)-5-(hydroxymethyl)-1H-pyrazol-1-yl)butan-2-ol. trans 2,3-Dimethyloxirane (2.5 mL, 28 mmol) was added to a solution consisting of 2-(5-(((tert-butyldimethylsilyl)oxy)methyl)-1H-pyrazol-3-yl)-5-fluoropyridine (Intermediate 35, 1.0 g, 3.3 mmol), Cs₂CO₃ (2.6 g, 8.0 mmol), and CH₃CN (50 mL). The resulting mixture was stirred at 70° C. for 16 hours. The reaction mixture was cooled to room-temperature. The suspension were filtered through a pad of Celite® and the pad washed with ethyl acetate (15 mL). The filtrate was concentrated to dryness under reduced pressure to afford the title compound (1.3 g) as a yellow oil, which was used in the next step without further purification. MS (ESI): mass calcd. for C₁₃H₁₆FN₃O₂ 265.1 m/z found 266.1 [M+H]⁺.

Step B. (Racemic) cis-2-(5-Fluoropyridin-2-yl)-6,7-dimethyl-6,7-dihydro-4H-pyrazolo[5,1-c][1,4]oxazine. 1-(3-(5-Fluoropyridin-2-yl)-5-(hydroxymethyl)-1H-pyrazol-1-yl)butan-2-ol (1.2 g, 4.5 mmol) was dissolved in H₃PO₄ (1.5 mL) and toluene (15 mL) in a 40 mL sealed tube. The resultant mixture was stirred at 110° C. for 16 hours. The reaction mixture was gradually cooled to room-temperature, then diluted with H₂O (20 mL). The pH was adjusted to pH=8 with 2 N NaOH, and extracted with ethyl acetate (30 mL×3). The combined organic extracts were washed with brine (15 mL), dried over anhydrous Na₂SO₄, filtered, and concentrated to dryness under reduced pressure. The resulting residue was purified by FCC (eluent: petroleum ether:ethyl acetate=1:0 to 3:1) to afford the title compound (100 mg, 8%) as a white solid. MS (ESI): mass calcd. for C₁₃H₁₄FN₃O₃ 247.1 m/z found 248.1 [M+H]⁺.

Step C. (Racemic) cis-3-Bromo-2-(5-fluoropyridin-2-yl)-6,7-dimethyl-6,7-dihydro-4H-pyrazolo[5,1-c][1,4]oxazine. NBS (144 mg, 0.809 mmol) was added to a solution consisting of cis-2-(5-fluoropyridin-2-yl)-6,7-dimethyl-6,7-dihydro-4H-pyrazolo[5,1-c][1,4]oxazine (200 mg, 0.809 mmol) and DMF (5 mL). The resultant mixture was stirred at room-temperature for 3 hours. The reaction mixture was quenched with sat·Na₂S₂O₅(20 mL) and extracted with ethyl acetate (10 mL×3). The combined organic extracts were dried over anhydrous Na₂SO₄, filtered, and concentrated to dryness under reduced pressure. The resulting residue was purified by FCC (SiO₂, eluent: petroleum ether:ethyl acetate=1:0 to 1:1) to afford the title compound (240 mg, 90%) as a white solid. MS (ESI): mass calcd. for C₁₃H₁₃BrFN₃O 325.0 m/z found 325.9 [M+H]⁺.

### Intermediate 90: (6*R,7*S)-3-Bromo-2-(5-fluoropyridin-2-yl)-6,7-dimethyl-6,7-dihydro-4H-pyrazolo[5,1-c][1,4]oxazine

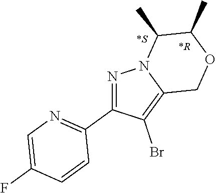 [SMILES: C[C@H]1OCc2c(Br)c(-c3ccc(F)cn3)nn2[C@H]1C]

(Racemic) cis-3-Bromo-2-(5-fluoropyridin-2-yl)-6,7-dimethyl-6,7-dihydro-4H-pyrazolo[5,1-c][1,4]oxazine (Intermediate 89, 240 mg, 0.736 mmol) was further purified by SFC Method B. The pure fractions were collected and the volatiles were removed under reduced pressure. The product was suspended in water (10 mL), the mixture frozen using dry ice/ethanol, and then lyophilized to dryness to afford (6*R,7*S)-3-bromo-2-(5-fluoropyridin-2-yl)-6,7-dimethyl-6,7-dihydro-4H-pyrazolo[5,1-c][1,4]oxazine (100 mg, 42%) as a white solid, MS (ESI): mass calcd. for C₁₃H₁₃BrFN₃O 325.0 m/z found 326.0 [M+H]⁺; ¹H NMR (400 MHz, DMSO-d₆) δ 8.64 (d, J=2.76 Hz, 1H), 7.87-7.97 (m, 1H), 7.75-7.85 (m, 1H), 4.66-4.88 (m, 2H), 4.28-4.38 (m, 1H), 4.10-4.20 (m, 1H), 1.22-1.33 (m, 6H); and (6*S,7*R)-3-bromo-2-(5-fluoropyridin-2-yl)-6,7-dimethyl-6,7-dihydro-4H-pyrazolo[5,1-c][1,4]oxazine (Intermediate 91) (120 mg, 50%) as a white solid.

### Intermediate 91: (6*S,7*R)-3-Bromo-2-(5-fluoropyridin-2-yl)-6,7-dimethyl-6,7-dihydro-4H-pyrazolo[5,1-c][1,4]oxazine

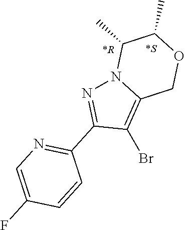 [SMILES: C[C@@H]1OCc2c(Br)c(-c3ccc(F)cn3)nn2[C@@H]1C]

The title compound was purified and isolated from Intermediate 89. MS (ESI): mass calcd. for C₁₃H₁₃BrFN₃O 325.0 m/z found 326.1 [M+H]⁺. ¹H NMR (400 MHz, DMSO-d₆) δ 8.64 (d, J=2.87 Hz, 1H), 7.87-8.03 (m, 1H), 7.74-7.84 (m, 1H), 4.64-4.88 (m, 2H), 4.28-4.37 (m, 1H), 4.09-4.18 (m, 1H), 1.23-1.32 (m, 6H).

### Intermediate 92: (Racemic) cis-3-Bromo 2-(5-Fluoropyridin-2-yl)-6,7-dimethyl-6,7-dihydro-4H-pyrazolo[5,1-c][1,4]oxazine

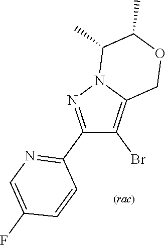 [SMILES: C[C@@H]1OCc2c(Br)c(-c3ccc(F)cn3)nn2[C@@H]1C]

Step A. Ethyl 3-(5-fluoropyridin-2-yl)-1-(3-oxobutan-2-yl)-1H-pyrazole-5-carboxylate. 3-Bromobutan-2-one (2.7 mL, 25 mmol) was added to a solution consisting of ethyl 3-(5-fluoropyridin-2-yl)-1H-pyrazole-5-carboxylate (Intermediate 34, 4.5 g, 19 mmol), Cs₂CO₃ (12.5 g, 38.4 mmol), and CH₃CN (100 mL). The resultant mixture was stirred at room-temperature for 16 hours. The mixture was concentrated to dryness under reduced pressure. The resulting residue was purified by FCC (SiO₂, eluent: petroleum ether:ethyl acetate=1:0 to 3:1) to afford the title compound (3.9 g, 63%) as a colourless oil. MS (ESI): mass calcd. for C₁₅H₁₆FN₃O₃ 305.1 m/z found 306.2 [M+H]⁺. ¹H NMR (400 MHz, DMSO-d₆) δ 8.60 (d, J=2.76 Hz, 1H), 8.00 (dd, J=4.52, 8.78 Hz, 1H), 7.73-7.84 (m, 1H), 7.39 (s, 1H), 5.82-5.94 (m, 1H), 4.26-4.35 (m, 2H), 2.08-2.19 (m, 3H), 1.72 (d, J=7.28 Hz, 3H), 1.26-1.37 (m, 3H).

Step B. 3-(3-(5-Fluoropyridin-2-yl)-5-(hydroxymethyl)-1H-pyrazol-1-yl)butan-2-ol. LiAlH₄ (1.5 g, 40 mmol) was added to a 0° C. (ice/water) solution consisting of ethyl 3-(5-fluoropyridin-2-yl)-1-(3-oxobutan-2-yl)-1H-pyrazole-5-carboxylate (3.9 g, 13 mmol) and THF (50 mL). The resultant mixture was stirred for 2 hours. The reaction mixture was gradually warmed to room-temperature, then quenched with H₂O (1.5 mL) and aq. NaOH (15 wt %, 1.5 mL) slowly. The mixture was stirred at room-temperature for 0.5 hours then further treated with H₂O (4.5 mL). The resultant mixture was stirred at room-temperature for another 0.5 hours and then dried over anhydrous MgSO₄. The suspension was filtered through a pad of Celite® and the pad washed with ethyl acetate (200 mL). The filtrate was concentrated to dryness under reduced pressure to afford the title compound (2.5 g), which was used in the next step without further purification. MS (ESI): mass calcd. for C₁₃H₁₆FN₃O₂ 265.1 m/z found 266.2 [M+H]⁺.

Step C. (Racemic) cis-2-(5-Fluoropyridin-2-yl)-6,7-dimethyl-6,7-dihydro-4H-pyrazolo[5,1-c][1,4]oxazine and (Racemic) trans-2-(5-Fluoropyridin-2-yl)-6,7-dimethyl-6,7-dihydro-4H-pyrazolo[5,1-c][1,4]oxazine. 3-(3-(5-Fluoropyridin-2-yl)-5-(hydroxymethyl)-1H-pyrazol-1-yl)butan-2-ol (2.5 g) was added to a 40 mL sealed tube containing a solution consisting of H₃PO₄ (3 mL) and toluene (30 mL). The resultant mixture was stirred at 130° C. for 6 hours. The reaction mixture was cooled to room-temperature. The reaction mixture was poured into H₂O (50 mL), the pH was adjusted pH=8 with 2 N NaOH and extracted with ethyl acetate (100 mL×3). The combined organic extracts were washed with brine (50 mL), dried over anhydrous Na₂SO₄, filtered, and concentrated to dryness under reduced pressure. The resulting residue was purified by FCC (SiO₂, eluent: petroleum ether:ethyl acetate=1:0 to 3:1) to afford (Racemic) cis-2-(5-fluoropyridin-2-yl)-6,7-dimethyl-6,7-dihydro-4H-pyrazolo[5,1-c][1,4]oxazine (400 mg, 15%) as a white solid; MS (ESI): mass calcd. for C₁₃H₁₄FN₃O 247.1 m/z found 248.1 [M+H]⁺; and (Racemic) trans-2-(5-fluoropyridin-2-yl)-6,7-dimethyl-6,7-dihydro-4H-pyrazolo[5,1-c][1,4]oxazine (500 mg, 17%) as a white solid; MS (ESI): mass calcd. for C₁₃H₁₄FN₃O 247.1 m/z found 248.1 [M+H]⁺.

Step D: (Racemic) cis-3-Bromo 2-(5-Fluoropyridin-2-yl)-6,7-dimethyl-6,7-dihydro-4H-pyrazolo[5,1-c][1,4]oxazine. The title compound was prepared in a manner analogous to Intermediate 37, Step B using (Racemic) cis-2-(5-fluoropyridin-2-yl)-6,7-dimethyl-6,7-dihydro-4H-pyrazolo[5,1-c][1,4]oxazine instead of 2-(5-fluoropyridin-2-yl)-6,7-dihydro-4H-pyrazolo[5,1-c][1,4]oxazine. MS (ESI): mass calcd. for C₁₃H₁₃BrFN₃O 325.0 m/z found 326.1 [M+H]⁺.

### Intermediate 93: (Racemic) trans-3-Bromo-2-(5-fluoropyridin-2-yl)-6,7-dimethyl-6,7-dihydro-4H-pyrazolo[5,1-c][1,4]oxazine

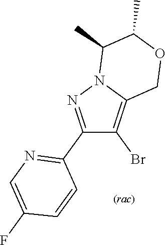 [SMILES: C[C@@H]1OCc2c(Br)c(-c3ccc(F)cn3)nn2[C@H]1C]

The title compound was prepared in a manner analogous to Intermediate 37, Step B using (Racemic) trans-2-(5-fluoropyridin-2-yl)-6,7-dimethyl-6,7-dihydro-4H-pyrazolo[5,1-c][1,4]oxazine (Intermediate 92, product from Step C) instead of 2-(5-fluoropyridin-2-yl)-6,7-dihydro-4H-pyrazolo[5,1-c][1,4]oxazine. MS (ESI): mass calcd. for C₁₃H₁₃BrFN₃O 325.1 m/z found 326.1 [M+H]⁺.

### Intermediate 94: 3-Bromo-2-(4-fluorophenyl)-7,7-dimethyl-6,7-dihydro-4H-pyrazolo[5,1-c][1,4]oxazine

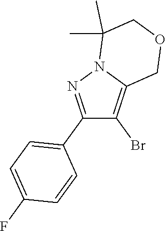 [SMILES: CC1(C)COCc2c(Br)c(-c3ccc(F)cc3)nn21]

Step A: Ethyl 1-(1-ethoxy-1-oxopropan-2-yl)-3-(4-fluorophenyl)-1H-pyrazole-5-carboxylate. Ethyl 2-bromopropanoate (3.7 g, 20 mmol) was added to a solution consisting of ethyl 3-(4-fluorophenyl)-1H-pyrazole-5-carboxylate (Intermediate 33, 4.0 g, 17 mmol), Cs₂CO₃ (13.6 g, 41.7 mmol) and MeCN (150 mL). The resultant mixture was stirred at room-temperature for 2 hours. The mixture was filtered through a pad of Celite® and the pad washed with ethyl acetate (150 mL). The filtrate was concentrated to dryness under reduced pressure. The resulting residue was purified by FCC (SiO₂, eluent: petroleum ether:ethyl acetate=1:0 to 10:1) to afford the title compound (4.9 g, 83%) as a colorless oil. LC-MS (ESI): mass calcd. for C₁₇H₁₉FN₂O₄ 334.13 m/z found 335.4 [M+H]⁺.

Step B: Ethyl 1-(1-ethoxy-2-methyl-1-oxopropan-2-yl)-3-(4-fluorophenyl)-1H-pyrazole-5-carboxylate. LiHMDS (14 mL, 14 mmol, 1M in THF) was added dropwise to a cooled (−70° C.; dry ice/ethanol) solution consisting of ethyl 1-(1-ethoxy-1-oxopropan-2-yl)-3-(4-fluorophenyl)-1H-pyrazole-5-carboxylate (2.8 g, 8.4 mmol) and THF (30 mL). The resultant mixture was stirred at −70° C. for 20 minutes and then Mel (20.0 g, 141 mmol) was added dropwise at −70° C. The resultant mixture was stirred for 16 hours. The reaction mixture was gradually warmed to room-temperature, then combined with additional batches and poured into sat·NH₄Cl (50 mL) and extracted with ethyl acetate (50 mL×3). The combined organic extracts were washed with brine (30 mL), dried over anhydrous Na₂SO₄, filtered, and concentrated to dryness under reduced pressure. The resulting residue was purified by FCC (SiO₂, eluent: petroleum ether:ethyl acetate=1:0 to 10:1) to afford the title compound (2.4 g. 82%) as a yellow solid. LC-MS (ESI): mass calcd. for C₁₈H₂₁FN₂O₄ 348.15 m/z found 349.1 [M+H]⁺.

Step C: 2-(3-(4-Fluorophenyl)-5-(hydroxymethyl)-1H-pyrazol-1-yl)-2-methylpropan-1-ol. LiAlH₄ (1.0 g, 26 mmol) was added in portions to a 100 mL three-necked round-bottomed flask containing a 0° C. (ice/water) solution consisting of ethyl 1-(1-ethoxy-2-methyl-1-oxopropan-2-yl)-3-(4-fluorophenyl)-1H-pyrazole-5-carboxylate (2.40 g, 6.89 mmol) and THF (30 mL) under N₂. The mixture was stirred under N₂ at room-temperature for 1 hour. The reaction mixture was quenched with water (5 mL) and 15% NaOH(aq) (5 mL). The mixture was filtered through Celite® pad and the filter cake washed with ethyl acetate (30 mL×3). The filtrate was washed with brine (30 mL×2), dried over anhydrous MgSO₄, filtered, and concentrated to dryness under reduced pressure to afford the title compound (1.8 g, crude), which was used directly for the next step without further purification. LC-MS (ESI): mass calcd. for C₁₄H₁₇FN₂O₂ 264.13 m/z found 265.2 [M+H]⁺.

Step D: 2-(4-Fluorophenyl)-7,7-dimethyl-6,7-dihydro-4H-pyrazolo[5,1-c][1,4]oxazine. 2-(3-(4-Fluorophenyl)-5-(hydroxymethyl)-1H-pyrazol-1-yl)-2-methylpropan-1-ol (1.8 g, 6.81 mmol) and conc. H₂SO₄ (12 mL) were added to a 100 mL round-bottomed flask. The reaction mixture was stirred at 90° C. for 16 hours. The reaction mixture was carefully added to water (150 mL) cooled with ice, then the mixture was adjusted to pH=7-8 with 15% aq. NaOH. The aqueous mixture was extracted with ethyl acetate (100 mL×3). The combined organic layers were dried over anhydrous Na₂SO₄, filtered, and concentrated to dryness under reduced pressure. The resulting residue was purified by FCC (SiO₂, eluent: petroleum ether:ethyl acetate=1:0 to 3:1) to afford the title compound (1 g, 60%) as a yellow solid. LC-MS (ESI): mass calcd. for C₁₄H₁₅FN₂O 246.12 m/z found 247.2 [M+H]⁺. ¹H NMR (400 MHz, DMSO-d₆) δ 7.84-7.73 (m, 2H), 7.25-7.15 (m, 2H), 6.42 (s, 1H), 4.82 (s, 2H), 3.81 (s, 2H), 1.46 (s, 6H).

Step D: 3-Bromo-2-(4-fluorophenyl)-7,7-dimethyl-6,7-dihydro-4H-pyrazolo[5,1-c][1,4]oxazine. The title compound was prepared in a manner analogous to Intermediate 37, Step B except using 2-(4-fluorophenyl)-7,7-dimethyl-6,7-dihydro-4H-pyrazolo[5,1-c][1,4]oxazine instead of 2-(5-fluoropyridin-2-yl)-6,7-dihydro-4H-pyrazolo[5,1-c][1,4]oxazine (Intermediate 37, Step A) and DCM instead of DMF. LC-MS (ESI): mass calcd. for C₁₄H₁₄BrFN₂O 324.03 m/z found 325.0 [M+H]⁺. ¹H NMR (400 MHz, DMSO-d₆) δ 7.87-7.79 (m, 2H), 7.34-7.26 (m, 2H), 4.76 (s, 2H), 3.86 (s, 2H), 1.47 (s, 6H).

### Intermediate 95: 3-Bromo-2-(5-fluoropyridin-2-yl)-7,7-dimethyl-6,7-dihydro-4H-pyrazolo[5,1-c][1,4]oxazine

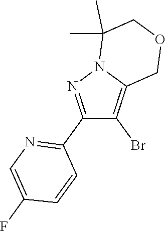 [SMILES: CC1(C)COCc2c(Br)c(-c3ccc(F)cn3)nn21]

The title compound was prepared in a manner analogous to Intermediate 94, Steps A-D except using ethyl 3-(5-fluoropyridin-2-yl)-1H-pyrazole-5-carboxylate (Intermediate 34) instead of ethyl 1-(1-ethoxy-1-oxopropan-2-yl)-3-(4-fluorophenyl)-1H-pyrazole-5-carboxylate (Intermediate 94, Step A) in Step A; LiBH₄ instead of LiAlH₄ in Step C; and DCM instead of DMF in Step D. ¹H NMR (400 MHz, DMSO-d₆) δ 8.65-8.63 (m, 1H), 7.95-7.90 (m, 1H), 7.83-7.77 (m, 1H), 4.77 (s, 2H), 3.88 (s, 2H), 1.48 (s, 6H).

### Intermediate 96: 3-Bromo-6,6-dimethyl-2-(thiazol-4-yl)-6,7-dihydro-4H-pyrazolo[5,1-c][1,4]oxazine

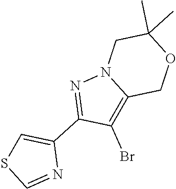 [SMILES: CC1(C)Cn2nc(-c3cscn3)c(Br)c2CO1]

Step A: 4-((Trimethylsilyl)ethynyl)thiazole. 4-Bromothiazole (2.0 g, 12.2 mmol), trimethylsilylacetylene (2.1 mL, 14.9 mmol), CuI (100 mg, 0.52 mmol) and triethylamine (8 mL) were added to a pressure vial. The mixture was sparged with N₂ for 5 minutes and then treated with Pd(PPh₃)₂C₁₂ (132 mg, 0.19 mmol). The vial was sealed, and the resultant mixture was heated at 85° C. via microwave irradiation for 1.5 h. The reaction mixture was cooled to room temp. The mixture was concentrated to dryness under reduced pressure and purified by FCC (SiO₂, eluent: (petroleum ether:ethyl acetate=1:0 to 10:1) to afford the title compound (2.3 g, 89%) as a yellow oil. MS (ESI): mass calcd. for C₈H₁₁NSSi 181.0 m/z found 182.1 [M+H]⁺.

Step B: 4-Ethynylthiazole. TBAF (14.0 mL, 14.0 mmol) was added to a solution of 4-((trimethylsilyl)ethynyl)thiazole (2.3 g, 12.7 mmol) in THF (40 mL). The solution was stirred at ambient temperature for 2 h. The resulting mixture was poured into water (50 mL), the layers were separated and the aqueous layer was extracted with DCM (50 mL, 2×). The combined organic extracts were dried over Na₂SO₄, filtered and evaporated. The crude residue was purified by FCC (SiO₂, eluent: petroleum ether:ethyl acetate=1:0 to 10:1) to afford the title compound (680 mg, 49%) as a colorless oil. ¹H NMR (400 MHz, DMSO-d₆) δ 9.12 (d, J=2.0 Hz, 1H), 8.10 (d, J=2.0 Hz, 1H), 4.39-4.14 (m, 1H)

Step C: 6,6-Dimethyl-2-(thiazol-4-yl)-6,7-dihydro-4H-pyrazolo[5,1-c][1,4]oxazine. The title compound was prepared in a manner analogous to Intermediate 37, Step A except using 4-ethynylthiazole instead of 2-ethynyl-5-fluoropyridine and 6,6-dimethyl-6,7-dihydro-4H-[1,2,3]oxadiazolo[4,3-c][1,4]oxazin-8-ium-3-olate (Intermediate 17) instead of 6,7-dihydro-4H-[1,2,3]oxadiazolo[4,3-c][1,4]oxazin-8-ium-3-olate (Intermediate 2). MS (ESI): mass calcd. for C₁₁H₁₃N₃OS 235.1 m/z found 236.0 [M+H]⁺. ¹H NMR (400 MHz, DMSO-d₆) δ 9.09 (d, J=2.0 Hz, 1H), 7.83 (d, J=2.0 Hz, 1H), 6.43 (s, 1H), 4.80 (s, 2H), 4.60 (s, 2H), 4.27 (s, 2H), 3.94 (s, 2H), 1.27 (d, J=3.7 Hz, 14H)

Step D: 3-Bromo-6,6-dimethyl-2-(thiazol-4-yl)-6,7-dihydro-4H-pyrazolo[5,1-c][1,4]oxazine. The title compound was prepared in a manner analogous to Intermediate 37, Step B except using 6,6-dimethyl-2-(thiazol-4-yl)-6,7-dihydro-4H-pyrazolo[5,1-c][1,4]oxazine (Intermediate 96, Step C) instead of 2-(5-fluoropyridin-2-yl)-6,7-dihydro-4H-pyrazolo[5,1-c][1,4]oxazine and 3-(5-fluoropyridin-2-yl)-6,7-dihydro-4H-pyrazolo[5,1-c][1,4]oxazine (Intermediate 37, Step A) except using DCM instead of DMF. MS (ESI): mass calcd. for C₁₁H₁₂BrN₃OS 313.0 m/z found 314.0 [M+H]⁺. ¹H NMR (400 MHz, DMSO-d₆) δ 9.16 (d, J=1.7 Hz, 1H), 8.00 (d, J=2.0 Hz, 1H), 4.72 (s, 2H), 3.97 (s, 2H), 1.27 (s, 6H).

### Intermediate 97: 3-Bromo-6,6-dimethyl-2-(oxazol-5-yl)-6,7-dihydro-4H-pyrazolo[5,1-c][1,4]oxazine

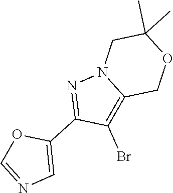 [SMILES: CC1(C)Cn2nc(-c3cnco3)c(Br)c2CO1]

Step A: Ethyl 6,6-dimethyl-6,7-dihydro-4H-pyrazolo[5,1-c][1,4]oxazine-2-carboxylate. A solution of ethyl propiolate (1.15 g, 11.72 mmol), 6,6-dimethyl-6,7-dihydro-4H-[1,2,3]oxadiazolo[4,3-c][1,4]oxazin-8-ium-3-olate (Intermediate 17, 1.5 g, 8.81 mmol) and xylenes (10 mL) was heated via microwave irradiation at 150° C. for 1 h. The reaction mixture was cooled to room temperature. Purification by FCC (eluent: petroleum ether:ethyl acetate=1:0 to 1:1) afforded 1.4 g (69%) of the title compound. MS (ESI): mass calcd. for C₁₁H₁₆N₂O₃, 224.1; m/z found, 225.3 [M+H]⁺. ¹H NMR (400 MHz, CDCl₃) δ 6.57 (s, 1H), 4.86 (s, 2H), 4.40 (q, J=7.1 Hz, 2H), 4.03 (s, 2H), 1.40 (t, J=7.1 Hz, 3H), 1.36 (s, 6H).

Step B: (6,6-Dimethyl-6,7-dihydro-4H-pyrazolo[5,1-c][1,4]oxazin-2-yl)methanol. MeOH (0.4 mL, 9.89 mmol) was added to a suspension of LiBH₄ (545 mg, 25.01 mmol) in THF (20 mL) at room temperature. Ethyl 6,6-dimethyl-6,7-dihydro-4H-pyrazolo[5,1-c][1,4]oxazine-2-carboxylate (1.4 g, 6.2 mmol) was added to the reaction and the resulting mixture was stirred at 40° C. for 16 h. The reaction was quenched with 1M HCl and the pH was adjusted to pH=1. The resulting mixture was stirred for 1 h at RT. The pH of the mixture was then adjusted to pH=8 with solid K₂CO₃ and the mixture was extracted with EtOAc (20 mL, ×2). The combined organic extracts were dried over anhydrous Na₂SO₄, filtered and concentrated to dryness under reduced pressure to afford the title compound (1.3 g) as a brown oil, which was used without further purification. MS (ESI): mass calcd. for C₉H₁₄N₂O₂, 182.1; m/z found, 182.9 [M+H]⁺.

Step C: 6,6-Dimethyl-6,7-dihydro-4H-pyrazolo[5,1-c][1,4]oxazine-2-carbaldehyde. MnO₂ (5.6 g, 64 mmol) was added to a solution of (6,6-dimethyl-6,7-dihydro-4H-pyrazolo[5,1-c][1,4]oxazin-2-yl)methanol (1.3 g, crude) and CHCl₃ (15 mL). The reaction was stirred at 60° C. for 1 h under N₂. The suspension was filtered through Celite® and the solvent was evaporated. The resulting residue was purified by FCC (SiO₂, eluent: petroleum ether:ethyl acetate=10:1 to 1:1) gave the title compound (900 mg, 98%). MS (ESI): mass calcd. for C₉H₁₂N₂O₂, 180.1; m/z found, 181.2 [M+H]⁺.

Step D: 6,6-Dimethyl-2-(oxazol-5-yl)-6,7-dihydro-4H-pyrazolo[5,1-c][1,4]oxazine. 6,6-dimethyl-6,7-dihydro-4H-pyrazolo[5,1-c][1,4]oxazine-2-carbaldehyde (900 mg, 4.99 mmol), 1-((isocyanomethyl)sulfonyl)-4-methylbenzene (1.5 g, 7.7 mmol), K₂CO₃ and MeOH (15 mL) were combined in a 40 mL flask. The resulting mixture was heated at 80° C. for 10 hrs. The suspension was filtered through Celite® and the pad was washed with MeOH (5 mL×3). The filtrate was concentrated to dryness under reduced pressure and then purified by FCC (eluent: petroleum ether:ethyl acetate=1:0 to 1:2) to afford the title compound (850 mg, 74%). MS (ESI): mass calcd. for C₁₁H₁₃N₃O₂, 219.1; m/z found, 220.2 [M+H]⁺. ¹H NMR (400 MHz, DMSO-d₆) δ 8.38 (s, 1H), 7.43 (s, 1H), 6.41 (s, 1H), 4.83 (s, 2H), 3.98 (s, 2H), 1.28 (s, 6H).

Step E: 3-Bromo-6,6-dimethyl-2-(oxazol-5-yl)-6,7-dihydro-4H-pyrazolo[5,1-c][1,4]oxazine. The title compound was prepared in a manner analogous to Intermediate 37, Step B except using 6,6-dimethyl-2-(oxazol-5-yl)-6,7-dihydro-4H-pyrazolo[5,1-c][1,4]oxazine instead of -(5-fluoropyridin-2-yl)-6,7-dihydro-4H-pyrazolo[5,1-c][1,4]oxazine and DCM instead of DMF. MS (ESI): mass calcd. for C₁₁H₁₂BrN₃O₂, 297.0; m/z found, 298.0 [M+H]⁺. ¹H NMR (400 MHz, CHLOROFORM-d) δ 7.96 (s, 1H), 7.67 (s, 1H), 4.79 (s, 2H), 3.99 (s, 2H), 1.39 (s, 6H).

### Intermediate 98: 3-Bromo-6,6-dimethyl-2-(1-methyl-1H-imidazol-4-yl)-6,7-dihydro-4H-pyrazolo[5,1-c][1,4]oxazine

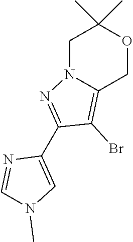 [SMILES: Cn1cnc(-c2nn3c(c2Br)COC(C)(C)C3)c1]

The title compound was prepared in a manner analogous to Intermediate 108, Steps A-B, except using 1-methyl-1H-imidazole-4-carbaldehyde instead of 5-chloro-6-methylpicolinaldehyde in Step A. MS (ESI): mass calcd. for C₁₂H₁₅BrN₄O 310.0 m/z found 311.1 [M+H]⁺. ¹H NMR (400 MHz, DMSO-d₆) δ 7.65-7.60 (m, 1H), 7.51 (d, J=1.1 Hz, 1H), 4.71 (s, 2H), 3.93 (s, 2H), 3.69 (s, 3H), 1.29 (s, 6H).

### Intermediate 99: 3-Bromo-2-(4-fluorophenyl)-6,6-dimethyl-6,7-dihydro-4H-pyrazolo[5,1-c][1,4]oxazine

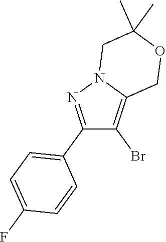 [SMILES: CC1(C)Cn2nc(-c3ccc(F)cc3)c(Br)c2CO1]

The title compound was prepared in a manner analogous to Intermediate 37, Steps A-B, except using 6,6-dimethyl-6,7-dihydro-4H-[1,2,3]oxadiazolo[4,3-c][1,4]oxazin-8-ium-3-olate (Intermediate 17) instead of 6,7-dihydro-4H-[1,2,3]oxadiazolo[4,3-c][1,4]oxazin-8-ium-3-olate (Intermediate 2) and using 1-ethynyl-4-fluorobenzene instead of 2-ethynyl-5-fluoropyridine in Step A. MS (ESI): mass calcd. for C₁₄H₁₄BrFN₂O, 324.0; m/z found, 325.0 [M+H]⁺. ¹H NMR (500 MHz, CDCl₃) δ 7.87 (t, J=6.59 Hz, 2H), 7.12 (t, J=8.33 Hz, 2H), 4.79 (s, 2H), 3.96 (s, 2H), 1.41 (s, 6H).

### Intermediate 100: 3-Bromo-2-(5-chloropyridin-2-yl)-6,6-dimethyl-6,7-dihydro-4H-pyrazolo[5,1-c][1,4]oxazine

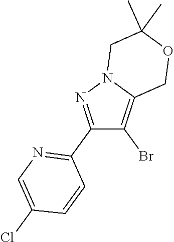 [SMILES: CC1(C)Cn2nc(-c3ccc(Cl)cn3)c(Br)c2CO1]

Step A. 5-Chloro-2-ethynylpyridine. A solution consisting of dimethyl (1-diazo-2-oxopropyl)phosphonate (4.99 g, 26.0 mmol) and methanol (20 mL) was added to a mixture consisting of 5-chloropicolinaldehyde (4.90 g, 34.6 mmol), K₂CO₃ (4.78 g, 34.6 mmol) and methanol (30 mL). The resulting mixture was stirred at room-temperature for 3 hours. The suspension was filtered through a pad of Celite® and the pad washed with ethyl acetate (300 mL). The filtrate was concentrated to dryness under reduced pressure. The resulting residue was purified by FCC (SiO₂, eluent: petroleum ether:ethyl acetate=1:0 to 10:1) to afford the title compound (2.7 g, 57%) as a light yellow solid. LCMS (ESI): mass calcd. for C₇H₄ClN 137.00 m/z, found 138.1 [M+H]⁺. ¹H NMR (400 MHz, CDCl₃) δ 8.55 (d, J=2.2 Hz, 1H), 7.66 (dd, J=2.4, 8.4 Hz, 1H), 7.44 (d, J=8.4 Hz, 1H), 3.21 (s, 1H).

Step B. 3-Bromo-2-(5-chloropyridin-2-yl)-6,6-dimethyl-6,7-dihydro-4H-pyrazolo[5,1-c][1,4]oxazine. The title compound was prepared in a manner analogous to Intermediate 37, Steps A-B, except using 6,6-dimethyl-6,7-dihydro-4H-[1,2,3]oxadiazolo[4,3-c][1,4]oxazin-8-ium-3-olate (Intermediate 17) instead of 6,7-dihydro-4H-[1,2,3]oxadiazolo[4,3-c][1,4]oxazin-8-ium-3-olate (Intermediate 2) and using 5-chloro-2-ethynylpyridine instead of 2-ethynyl-5-fluoropyridine, also microwave heating for 1 hr instead of conventional heating for 16 hrs in Step A. LCMS (ESI): mass calcd. for C₁₃H₁₃BrClN₃O 341.0 m/z, found 342.0 [M+H]⁺. ¹H NMR (400 MHz, CDCl₃) δ 8.68 (br s, 1H), 7.97 (d, J=8.4 Hz, 1H), 7.74 (dd, J=2.5, 8.5 Hz, 1H), 4.80 (s, 2H), 4.01 (s, 2H), 1.40 (s, 6H).

### Intermediate 101: 3-Bromo-2-(5-fluoropyridin-2-yl)-6,6-dimethyl-6,7-dihydro-4H-pyrazolo[5,1-c][1,4]oxazine

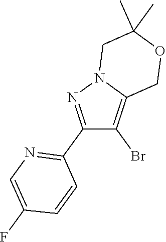 [SMILES: CC1(C)Cn2nc(-c3ccc(F)cn3)c(Br)c2CO1]

The title compound was prepared in a manner analogous to Intermediate 37, Steps A-B, except using 6,6-dimethyl-6,7-dihydro-4H-[1,2,3]oxadiazolo[4,3-c][1,4]oxazin-8-ium-3-olate (Intermediate 17) instead of 6,7-dihydro-4H-[1,2,3]oxadiazolo[4,3-c][1,4]oxazin-8-ium-3-olate (Intermediate 2) and microwave heating at 155° C. for 1 hr instead of conventional heating for 16 hrs in Step A and DCM instead of DMF in Step B. ¹H NMR (400 MHz, CDCl₃) δ 8.58 (d, J=2.8 Hz, 1H), 8.01 (dd, J=4.5, 8.8 Hz, 1H), 7.48 (dt, J=3.0, 8.4 Hz, 1H), 4.80 (s, 2H), 4.01 (s, 2H), 1.39 (s, 6H).

### Intermediate 102: 4-(3-Bromo-4,5,6,7-tetrahydropyrazolo[1,5-a]pyridin-2-yl)thiazole

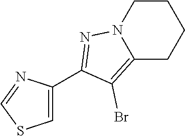 [SMILES: Brc1c(-c2cscn2)nn2c1CCCC2]

The title compound was prepared in a manner analogous to Intermediate 37, Steps A-B, except using 4,5,6,7-tetrahydro-[1,2,3]oxadiazolo[3,4-a]pyridin-8-ium-3-olate (Intermediate 9) and 4-ethynylthiazole in Step A. MS (ESI): mass calcd. For C₁₀H₁₀BrN₃S, 282.97 m/z found, 283.9 [M+H]⁺. ¹H NMR (400 MHz, DMSO-d₆): δ 9.19 (s, 1H), 7.99 (s, 1H), 4.11 (t, J=6.0 Hz, 2H), 2.68 (t, J=6.4 Hz, 2H), 2.03-1.97 (m, 2H), 1.88-1.80 (m, 2H).

### Intermediate 103: 3-Bromo-2-(5-fluoropyridin-2-yl)-6,6-dimethyl-6,7-dihydro-4H-pyrazolo[5,1-c][1,4]oxazine-4,4-d₂

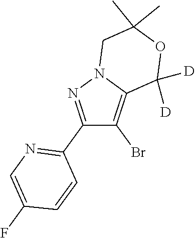 [SMILES: [2H]C1([2H])OC(C)(C)Cn2nc(-c3ccc(F)cn3)c(Br)c21]

Step A. 1-(3-(5-Fluoropyridin-2-yl)-5-(hydroxymethyl-d₂)-1H-pyrazol-1-yl)-2-methylpropan-2-ol. 2,2-Dimethyloxirane (10.0 mL, 112 mmol) was added to a solution consisting of di-D-(3-(5-fluoropyridin-2-yl)-1H-pyrazol-5-yl)methanol (1.0 g, crude), Cs₂CO₃ (4.0 g, 12 mmol), and CH₃CN (30 mL). The resultant mixture was stirred at 70° C. for 16 hours. The reaction mixture was cooled to room-temperature. The suspension was filtered through a pad of Celite® and the pad washed with ethyl acetate (40 mL). The filtrate was concentrated to dryness under reduced pressure to afford the title compound (1.0 g, crude) as a yellow semi-solid, which was used in the next step without further purification. LC-MS (ESI): mass calcd. for C₁₃H₁₄D₂FN₃O₂ 267.14 m/z found 267.9 [M+H]⁺.

Step B. 2-(5-Fluoropyridin-2-yl)-6,6-dimethyl-6,7-dihydro-4H-pyrazolo[5,1-c][1,4]oxazine-4,4-d₂. Conc. H₂SO₄ (15 mL) was added to a 0° C. (ice/water) solution consisting of 1-(3-(5-fluoropyridin-2-yl)-5-(hydroxymethyl-d₂)-1H-pyrazol-1-yl)-2-methylpropan-2-ol (1.0 g, crude) and dichloromethane (15 mL). The resultant mixture was heated at 70° C. for 2 hours. The reaction mixture was gradually cooled to room-temperature, poured it into ice water (60 mL), and the pH was adjusted to pH=6 with 4 N NaOH. The resultant mixture was extracted with ethyl acetate (100 mL×3). The combined organic extracts were dried over anhydrous Na₂SO₄, filtered, and concentrated to dryness under reduced pressure. The resulting residue was purified by FCC (SiO₂, eluent: petroleum ether:ethyl acetate=10:1 to 1:1) to afford the title compound (160 mg) as a yellow solid. LC-MS (ESI): mass calcd. for C₁₃H₁₂D₂FN₃O 249.12 m/z found 250.5 [M+H]⁺. ¹H NMR (400 MHz, CDCl₃): δ 8.47 (d, J=2.9 Hz, 1H), 7.90 (dd, J=4.5, 8.8 Hz, 1H), 7.43 (dt, J=2.9, 8.5 Hz, 1H), 6.58 (s, 1H), 4.03 (s, 2H), 1.40 (s, 6H).

Step C. 3-Bromo-2-(5-fluoropyridin-2-yl)-6,6-dimethyl-6,7-dihydro-4H-pyrazolo[5,1-c][1,4]oxazine-4,4-d₂. The title compound was prepared in a manner analogous to Intermediate 37, Step B, except using 2-(5-fluoropyridin-2-yl)-6,6-dimethyl-6,7-dihydro-4H-pyrazolo[5,1-c][1,4]oxazine-4,4-d₂ instead of 2-(5-fluoropyridin-2-yl)-6,7-dihydro-4H-pyrazolo[5,1-c][1,4]oxazine and 3-(5-fluoropyridin-2-yl)-6,7-dihydro-4H-pyrazolo[5,1-c][1,4]oxazine; and DCM instead of DMF. LC-MS (ESI): mass calcd. for C₁₃H₁₁BrD₂FN₃O 327.04 m/z found 328.0 [M+H]⁺.

### Intermediate 104: 4-(2-(5-Fluoropyridin-2-yl)-6,6-dimethyl-6,7-dihydro-4H-pyrazolo[5,1-c][1,4]oxazin-3-yl)pyridin-2-amine

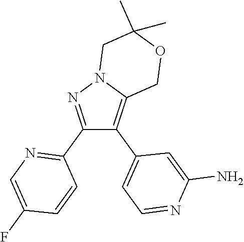 [SMILES: CC1(C)Cn2nc(-c3ccc(F)cn3)c(-c3ccnc(N)c3)c2CO1]

3-Bromo-2-(5-fluoropyridin-2-yl)-6,6-dimethyl-6,7-dihydro-4H-pyrazolo[5,1-c][1,4]oxazine (Intermediate 101, 400 mg, 1.23 mmol), 2-aminopyridine-4-boronic acid pinacol ester (337 mg, 1.53 mmol), Cs₂CO₃ (1.21 g, 3.68 mmol) and CataCXium® A Pd G3 (45 mg, 0.06 mmol) were combined in 2-methyl-2-butanol (9.8 mL) and water (2.4 mL). Nitrogen was bubbled through the reaction mixture for 2 minutes the resulting mixture was heated to 90° C. for 16 hours. The reaction mixture was extracted with EtOAc (3×25 mL) and the combined organic layers were dried over Na₂SO₄, filtered and evaporated. The resulting residue was purified by FCC (SiO₂, 10% MeOH containing 2M NH₃ in DCM, 0-50%) gave the title compound (174 mg, 42%). MS (ESI): mass calcd. for C₁₈H₁₈FN₅O, 339.1; m/z found, 340.1 [M+H]⁺.

### Intermediate 105: Lithium 2-(2-(5-fluoropyridin-2-yl)-6,6-dimethyl-6,7-dihydro-4H-pyrazolo[5,1-c][1,4]oxazin-3-yl)-2-hydroxy-4,4,5,5-tetramethyl-1,3,2-dioxaborolan-2-uide

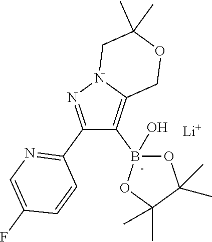

To a solution of 3-bromo-2-(5-fluoropyridin-2-yl)-6,6-dimethyl-6,7-dihydro-4H-pyrazolo[5,1-c][1,4]oxazine (Intermediate 101), 331 mg, 1 mmol) and 2-isopropoxy-4,4,5,5-tetramethyl-1,3,2-dioxaborolane (0.27 mL, 1.3 mmol) in THF (2 mL) and toluene (2 mL), cooled to −78° C., was added n-butyllithium (0.95 mL of 1.6 M in hexanes, 1.5 mmol) dropwise. After 2 h the reaction was warmed to room temperature then pinacol (36 mg, 0.3 mmol) and water (0.056 mL, 3 mmol) were added. The resulting precipitate was collected, rinsed with Et₂O, and air dried to afford 260 mg (64%) of the title compound. MS (ESI): mass calcd. for C₁₃H₁₅BFN₃O₃, 291.1; m/z found, 292.1 [M+H]⁺. ¹H NMR (400 MHz, DMSO-d₆) δ 8.48 (br dd, J=8.63, 4.88 Hz, 1H), 8.40 (d, J=2.75 Hz, 1H), 7.60 (td, J=8.85, 3.06 Hz, 1H), 4.92 (s, 2H), 3.85 (s, 2H), 1.25 (s, 6H), 1.02 (s, 6H), 0.84 (s, 6H).

### Intermediate 106: 3-Bromo-2-(6-methoxypyridin-2-yl)-6,6-dimethyl-6,7-dihydro-4H-pyrazolo[5,1-c][1,4]oxazine

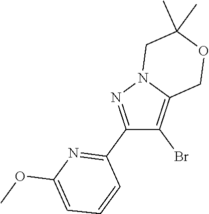 [SMILES: COc1cccc(-c2nn3c(c2Br)COC(C)(C)C3)n1]

The title compound was prepared in a manner analogous to Intermediate 37, Steps A-B, except using 6,6-dimethyl-6,7-dihydro-4H-[1,2,3]oxadiazolo[4,3-c][1,4]oxazin-8-ium-3-olate (Intermediate 17) instead of 6,7-dihydro-4H-[1,2,3]oxadiazolo[4,3-c][1,4]oxazin-8-ium-3-olate (Intermediate 2) and using ethynyl-6-methoxypyridine instead of 2-ethynyl-5-fluoropyridine, also microwave heating at 170° C. for 1.5 hr instead of conventional heating for 16 hrs in Step A. MS (ESI): mass calcd. for C₁₄H₁₆BrN₃O₂ 337.0 m/z, found 337.9 [M+H]⁺. ¹H NMR (400 MHz, DMSO-d₆) δ 7.81-7.70 (m, 1H), 7.49-7.44 (m, 1H), 6.79 (d, J=8.0 Hz, 1H), 4.75 (s, 2H), 4.03-4.02 (m, 2H), 3.96-3.94 (m, 3H), 1.31 (s, 6H).

### Intermediate 107: 3-Bromo-2-(3-chloropyridin-4-yl)-6,6-dimethyl-6,7-dihydro-4H-pyrazolo[5,1-c][1,4]oxazine

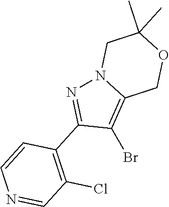 [SMILES: CC1(C)Cn2nc(-c3ccncc3Cl)c(Br)c2CO1]

The title compound was prepared in a manner analogous to Intermediate 100, Step A-B except using 3-chloroisonicotinaldehyde instead of 5-chloropicolinaldehyde in Step A. MS (ESI): mass calcd. for C₁₃H₁₃BrClN₃O 341.0 m/z, found 342.1 [M+H]⁺.

### Intermediate 108: 3-Bromo-2-(5-chloro-6-methylpyridin-2-yl)-6,6-dimethyl-6,7-dihydro-4H-pyrazolo[5,1-c][1,4]oxazine

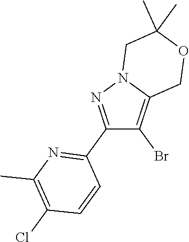 [SMILES: Cc1nc(-c2nn3c(c2Br)COC(C)(C)C3)ccc1Cl]

Step A. 3-Chloro-6-ethynyl-2-methylpyridine. Dimethyl (1-diazo-2-oxopropyl)phosphonate (0.988 g, 5.14 mmol) and MeOH (3 mL) were added to a mixture consisting of 5-chloro-6-methylpicolinaldehyde (500 mg, 3.21 mmol), K₂CO₃ (1.109 g, 8.023 mmol), and MeOH (8 mL). The resultant mixture was stirred at room-temperature for 3 hours. The suspension was filtered through a pad of Celite® and the pad washed with MeOH (20 mL×2). The filtrate was concentrated to dryness under reduced pressure. The resulting residue was purified by FCC (SiO₂, eluent: petroleum ether:ethyl acetate=0:1 to 3:1) to afford the title compound (233 mg, 47%) as a brown oil. ¹H NMR (400 MHz, DMSO-d₆) δ 7.88 (d, J=8.2 Hz, 1H), 7.44 (d, J=8.4 Hz, 1H), 4.39 (s, 1H), 2.52 (s, 3H).

Step B. 3-Bromo-2-(5-chloro-6-methylpyridin-2-yl)-6,6-dimethyl-6,7-dihydro-4H-pyrazolo[5,1-c][1,4]oxazine. The title compound was prepared in a manner analogous to Intermediate 37, Steps A-B, except using 6,6-dimethyl-6,7-dihydro-4H-[1,2,3]oxadiazolo[4,3-c][1,4]oxazin-8-ium-3-olate (Intermediate 17) instead of 6,7-dihydro-4H-[1,2,3]oxadiazolo[4,3-c][1,4]oxazin-8-ium-3-olate (Intermediate 2) and using chloro-6-ethynyl-2-methylpyridine instead of 2-ethynyl-5-fluoropyridine, also microwave heating at 170° C. for 2 hr instead of conventional heating for 16 hrs in Step A. LC-MS (ESI): mass calcd. for C₁₄H₁₅BrClN₃O 355.01 m/z, found 355.9 [M+H]⁺. ¹H NMR (400 MHz, CDCl₃): δ 7.80-7.74 (m, 1H), 7.71-7.67 (m, 1H), 4.79 (s, 2H), 4.02 (s, 2H), 2.72 (s, 3H), 1.39 (s, 6H).

### Intermediate 109: 3-Bromo-2-(5-fluoro-6-methylpyridin-2-yl)-6,6-dimethyl-6,7-dihydro-4H-pyrazolo[5,1-c][1,4]oxazine

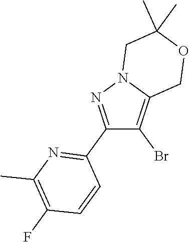 [SMILES: Cc1nc(-c2nn3c(c2Br)COC(C)(C)C3)ccc1F]

The title compound was prepared in a manner analogous to Intermediate 108, Steps A-B, except using 5-fluoro-6-methylpicolinaldehyde instead of 5-chloro-6-methylpicolinaldehyde in Step A. MS (ESI): mass calcd. for C₁₄H₁₅BrFN₃O 339.04 m/z found 339.8 [M+H]⁺.

### Intermediate 110: 3-Bromo-2-(3,5-difluoropyridin-4-yl)-6,6-dimethyl-6,7-dihydro-4H-pyrazolo[5,1-c][1,4]oxazine

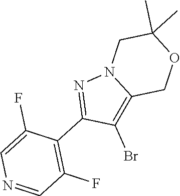 [SMILES: CC1(C)Cn2nc(-c3c(F)cncc3F)c(Br)c2CO1]

The title compound was prepared in a manner analogous to Intermediate 37, Steps A-B, except using 6,6-dimethyl-6,7-dihydro-4H-[1,2,3]oxadiazolo[4,3-c][1,4]oxazin-8-ium-3-olate (Intermediate 17) instead of 6,7-dihydro-4H-[1,2,3]oxadiazolo[4,3-c][1,4]oxazin-8-ium-3-olate (Intermediate 2) and 4-ethynyl-3,5-difluoropyridine instead of 2-ethynyl-5-fluoropyridine, also microwave heating at 150° C. for 1 hr instead of conventional heating for 16 hrs in Step A and DCM instead of DMF n Step B. MS (ESI): mass calcd. for C₁₃H₁₂BrF₂N₃O 343.0 m/z, found 343.9[M+H]⁺. ¹H NMR (400 MHz, CDCl₃): δ 8.47 (s, 2H), 4.82 (s, 2H), 4.03 (s, 2H), 1.42 (s, 6H).

### Intermediate 111: 3-Bromo-2-(3,5-difluoropyridin-2-yl)-6,6-dimethyl-6,7-dihydro-4H-pyrazolo[5,1-c][1,4]oxazine

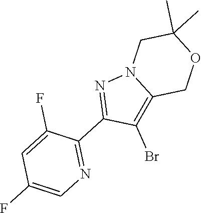 [SMILES: CC1(C)Cn2nc(-c3ncc(F)cc3F)c(Br)c2CO1]

The title compound was prepared in a manner analogous to Intermediate 37, Steps A-B, except using 6,6-dimethyl-6,7-dihydro-4H-[1,2,3]oxadiazolo[4,3-c][1,4]oxazin-8-ium-3-olate (Intermediate 17) instead of 6,7-dihydro-4H-[1,2,3]oxadiazolo[4,3-c][1,4]oxazin-8-ium-3-olate (Intermediate 2) and using 2-ethynyl-3,5-difluoropyridine instead of 2-ethynyl-5-fluoropyridine, also microwave heating at 155° C. for 1 hr instead of conventional heating for 16 hrs in Step A and DCM instead of DMF in Step B. MS (ESI): mass calcd. for C₁₃H₁₂BrF₂N₃O 343.0 m/z, found 343.9 [M+H]⁺. ¹H NMR (400 MHz, DMSO-d₆) δ 8.64 (d, J=2.3 Hz, 1H), 8.17-8.03 (m, 1H), 4.78 (s, 2H), 4.04 (s, 2H), 1.31 (s, 6H).

### Intermediate 112: 3-Bromo-6-ethyl-2-(5-fluoropyridin-2-yl)-6-methyl-6,7-dihydro-4H-pyrazolo[5,1-c][1,4]oxazine

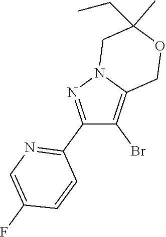 [SMILES: CCC1(C)Cn2nc(-c3ccc(F)cn3)c(Br)c2CO1]

Step A. 1-(5-(((tert-Butyldimethylsilyl)oxy)methyl)-3-(5-fluoropyridin-2-yl)-1H-pyrazol-1-yl)butan-2-one. 1-Bromobutan-2-one (640 mg, 4.24 mmol) was added to a solution consisting of 2-(5-(((tert-butyldimethylsilyl)oxy)methyl)-1H-pyrazol-3-yl)-5-fluoropyridine (Intermediate 35, 1.0 g, 3.3 mmol), Cs₂CO₃ (2.12 g, 6.51 mmol), and CH₃CN (15 mL). The resultant mixture was stirred at room-temperature for 16 hours. The reaction mixture was poured into H₂O (20 mL) and extracted with ethyl acetate (20 mL×2). The combined organic extracts were dried over anhydrous Na₂SO₄, filtered, and concentrated to dryness under reduced pressure. The resulting residue was purified by FCC (SiO₂, eluent: petroleum ether:ethyl acetate=1:0 to 2:1) to afford the title compound (1.3 g, 65%) as a yellow oil. LC-MS (ESI): mass calcd. for C₁₉H₂₈FN₃O₂Si 377.19 m/z found 378.9 [M+H]⁺.

Step B. 1-(5-(((tert-Butyldimethylsilyl)oxy)methyl)-3-(5-fluoropyridin-2-yl)-1H-pyrazol-1-yl)-2-methylbutan-2-ol. MeLi (5.25 mL, 1.6 M in Et₂O 8.40 mmol) was added drop wise to a −70° C. (dry ice/EtOH) solution consisting of 1-(5-(((tert-butyldimethylsilyl)oxy)methyl)-3-(5-fluoropyridin-2-yl)-1H-pyrazol-1-yl)butan-2-one (1.15 g, 1.86 mmol) and dichloromethane (15 mL). The mixture was stirred for 1 hour. The reaction mixture was gradually warmed to room-temperature and then stirred at room-temperature for 9 hours. The reaction mixture was quenched with sat·NH₄Cl (30 mL) and extracted with ethyl acetate (30 mL×3). The combined organic extracts were washed with brine (20 mL), dried over anhydrous Na₂SO₄, filtered, and concentrated to dryness under reduced pressure. The resulting residue was purified by FCC (SiO₂, eluent: petroleum ether:ethyl acetate=1:0 to 3:1) to afford title compound (700 mg, 89%) as a yellow oil. LC-MS (ESI): mass calcd. for C₂₀H₃₂FN₃O₂Si 393.22 m/z found 394.1 [M+H]⁺.

Step C. 1-(3-(5-Fluoropyridin-2-yl)-5-(hydroxymethyl)-1H-pyrazol-1-yl)-2-methylbutan-2-ol. TBAF (3.5 mL, 1 M in THF, 3.5 mmol) was added to a solution consisting of 1-(5-(((tert-butyldimethylsilyl)oxy)methyl)-3-(5-fluoropyridin-2-yl)-1H-pyrazol-1-yl)-2-methylbutan-2-ol (700 mg, 1.78 mmol) and THF (10 mL). The resultant solution was stirred for 1 hour at room-temperature. The reaction mixture was quenched with sat. NH₄Cl (20 mL) and extracted with ethyl acetate (15 mL×3). The combined organic extracts were washed with brine (15 mL), dried over anhydrous Na₂SO₄, filtered, and concentrated to dryness under reduced pressure. The resulting residue was purified by FCC (SiO₂, eluent: petroleum ether:ethyl acetate=1:0 to 1:2) to afford the title compound (490 mg, 97%) as a yellow oil. LC-MS (ESI): mass calcd. for C₁₄H₁₈FN₃O₂ 279.14 m/z found 280.1 [M+H]⁺.

Step D. 6-Ethyl-2-(5-fluoropyridin-2-yl)-6-methyl-6,7-dihydro-4H-pyrazolo[5,1-c][1,4]oxazine. H₃PO₄ (1.5 mL) was added into a solution consisting of 1-(3-(5-fluoropyridin-2-yl)-5-(hydroxymethyl)-1H-pyrazol-1-yl)-2-methylbutan-2-ol (460 mg, 1.65 mmol) and toluene (15 mL). The resultant mixture was stirred at 110° C. for 16 hours. The reaction mixture was poured into H₂O (20 mL), the pH was adjusted to pH=8 with 3 M NaOH and extracted with ethyl acetate (20 mL×3). The combined organic extracts were washed with brine (15 mL), dried over anhydrous Na₂SO₄, filtered, and concentrated to dryness under reduced pressure to afford the title compound (390 mg, crude), which was used in the next step without further purification. LC-MS (ESI): mass calcd. for C₁₄H₁₆FN₃O 261.13 m/z found 261.9 [M+H]⁺.

Step E. 3-Bromo-6-ethyl-2-(5-fluoropyridin-2-yl)-6-methyl-6,7-dihydro-4H-pyrazolo[5,1-c][1,4]oxazine. NBS (265 mg, 1.49 mmol) was added to a solution consisting of 6-ethyl-2-(5-fluoropyridin-2-yl)-6-methyl-6,7-dihydro-4H-pyrazolo[5,1-c][1,4]oxazine (390 mg, 1.49 mmol) and dichloromethane (5 mL). The resultant mixture was stirred at room-temperature for 1.5 hours. The reaction mixture was quenched with H₂O (15 mL) and extracted with dichloromethane (10 mL×2). The combined organic extracts were dried over anhydrous Na₂SO₄, filtered, and concentrated to dryness under reduced pressure afford the crude compound, which was purified by FCC (SiO₂, eluent: petroleum ether:ethyl acetate=1:0 to 1:1) to afford the title compound (300 mg) as a yellow oil. LC-MS (ESI): mass calcd. for C₁₄H₁₅BrFN₃O 339.1 m/z found 339.7 [M+H]⁺.

### Intermediate 113: 3-Bromo-2-(4-fluorophenyl)-6,6,7-trimethyl-6,7-dihydro-4H-pyrazolo[5,1-c][1,4]oxazine

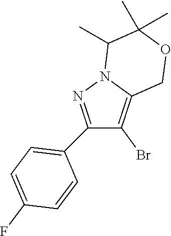 [SMILES: CC1n2nc(-c3ccc(F)cc3)c(Br)c2COC1(C)C]

Step A: 5-(((tert-Butyldimethylsilyl)oxy)methyl)-3-(4-fluorophenyl)-1H-pyrazole. TBSCl (6.6 g, 44 mmol) was added to a solution consisting of (3-(4-fluorophenyl)-1H-pyrazol-5-yl)methanol (Intermediate 70, product from Step A, 5.60 g, 29.1 mmol), imidazole (6.00 g, 87.4 mmol), dichloromethane (40 mL), and DMF (8 mL). The resultant mixture was stirred at room-temperature for 30 minutes. The suspension was filtered through a pad of Celite® and the pad washed with ethyl acetate (100 mL). The filtrate was concentrated to dryness under reduced pressure. The residue was diluted with water (100 mL) and the resultant mixture extracted with ethyl acetate (100 mL×3). The combined organic extracts were washed with brine (50 mL), dried over anhydrous Na₂SO₄, filtered, and concentrated to dryness under reduced pressure. The resulting residue was purified by FCC (SiO₂, eluent: petroleum ether:ethyl acetate=0:1 to 5:1) to afford the title compound (7.22 g, 75%) as a colorless oil. LC-MS (ESI): mass calcd. for C₁₆H₂₃FN₂OSi 306.16 m/z found 307.1 [M+H]⁺. ¹H NMR (400 MHz, CDCl₃): δ 10.62 (br s, 1H), 7.84-7.58 (m, 2H), 7.18-6.97 (m, 2H), 6.40 (s, 1H), 4.82 (s, 2H), 1.02-0.83 (m, 9H), 0.23-0.00 (m, 6H).

Step C: 3-(5-(((tert-Butyldimethylsilyl)oxy)methyl)-3-(4-fluorophenyl)-1H-pyrazol-1-yl)butan-2-one. 3-Bromobutan-2-one (2.176 mL, 20.7 mmol) was added to a solution consisting of 5-(((tert-butyldimethylsilyl)oxy)methyl)-3-(4-fluorophenyl)-1H-pyrazole (5.0 g, 16 mmol), Cs₂CO₃ (10.6 g, 32.6 mmol), and MeCN (50 mL). The resultant mixture was stirred at room-temperature for 2 hours. The suspension was filtered through a pad of Celite® and the pad washed with ethyl acetate (50 mL). The filtrate was concentrated to dryness under reduced pressure. The resulting residue was purified by FCC (SiO₂, eluent: petroleum ether:ethyl acetate=1:0 to 15:1) to afford the title compound (5.6 g, 89%) as a colorless oil. LC-MS (ESI): mass calcd. for C₂₀H₂₉FN₂O₂Si 376.20 m/z found 377.5 [M+H]⁺. ¹H NMR (400 MHz, CDCl₃): δ 7.87-7.69 (m, 2H), 7.13-7.00 (m, 2H), 6.52-6.38 (m, 1H), 4.98 (q, J=7.0 Hz, 1H), 4.78-4.62 (m, 2H), 2.02-1.90 (m, 3H), 1.74 (d, J=7.0 Hz, 3H), 0.98-0.79 (m, 9H), 0.12 (d, J=5.5 Hz, 6H).

Step D: 3-(5-(((tert-Butyldimethylsilyl)oxy)methyl)-3-(4-fluorophenyl)-1H-pyrazol-1-yl)-2-methylbutan-2-ol. MeLi (46 mL, 1.6 M in hexane, 74 mmol) was added dropwise to a (−70° C., dry ice/EtOH) solution consisting of 3-(5-(((tert-butyldimethylsilyl)oxy)methyl)-3-(5-fluoropyridin-2-yl)-1H-pyrazol-1-yl)butan-2-one (5.6 g, 15 mmol) and THF (60 mL) under N₂ atmosphere. The resultant mixture was stirred for 3 hours at −70° C. (dry ice/EtOH). The reaction mixture was quenched with sat. NH₄Cl (60 mL) and extracted with ethyl acetate (60 mL×3). The combined organic extracts were dried over anhydrous Na₂SO₄, filtered, and concentrated to dryness under reduced pressure. The resulting residue was purified by FCC (petroleum ether:ethyl acetate=1:0 to 10:1) to afford the still-impure product (5.3 g) as a colorless oil. The still-impure product was further purified by preparative HPLC Method B to afford pure product. The product was suspended in water (10 mL), the mixture frozen using dry ice/EtOH, and then lyophilized to dryness to afford the title compound (4.4 g, 75%) as a yellow oil. LC-MS (ESI): mass calcd. for C₂₁H₃₃FN₂O₂Si 392.23 m/z found 393.3 [M+H]⁺.

Step E: 3-(3-(4-Fluorophenyl)-5-(hydroxymethyl)-1H-pyrazol-1-yl)-2-methylbutan-2-ol. TBAF (22.4 mL, 1 M in THF, 22.4 mmol) was added dropwise to a 0° C. (ice/water) solution consisting of 3-(5-(((tert-butyldimethylsilyl)oxy)methyl)-3-(5-fluoropyridin-2-yl)-1H-pyrazol-1-yl)-2-methylbutan-2-ol (4.4 g, 11 mmol) and THF (30 mL). The resultant mixture was stirred at room-temperature for 1 hour. The reaction mixture was poured into brine (30 mL) and extracted with ethyl acetate (30 mL×3). The combined organic extracts were washed with brine (20 mL×2), dried over anhydrous Na₂SO₄, filtered, and concentrated to dryness under reduced pressure to give the title compound (5 g), which was used in the next step without further purification.

Step F: 2-(4-Fluorophenyl)-6,6,7-trimethyl-6,7-dihydro-4H-pyrazolo[5,1-c][1,4]oxazine. 3-(3-(4-Fluorophenyl)-5-(hydroxymethyl)-1H-pyrazol-1-yl)-2-methylbutan-2-ol (2 g, crude) was added to a solution consisting of H₃PO₄ (2 mL) and toluene (20 mL). The resultant mixture was heated at 130° C. for 16 hours. The reaction mixture was cooled to room-temperature. The reaction mixture was poured into sat·NaHCO₃(60 mL) and extracted with ethyl acetate (70 mL×3). The combined organic extracts were washed with brine (60 mL), dried over anhydrous Na₂SO₄, filtered, and concentrated to dryness under reduced pressure. The resulting residue was purified by FCC (SiO₂, eluent: petroleum ether:ethyl acetate=10:1 to 4:1) to afford the crude title compound (550 mg, crude) as a colorless oil, which was used in the next step without further purification.

Step F: 3-Bromo-2-(4-fluorophenyl)-6,6,7-trimethyl-6,7-dihydro-4H-pyrazolo[5,1-c][1,4]oxazine. 2-(4-Fluorophenyl)-6,6,7-trimethyl-6,7-dihydro-4H-pyrazolo[5,1-c][1,4]oxazine (550 mg, 2.11 mmol), NBS (564 mg, 3.17 mmol), and dichloromethane (10 mL) were added to a 50 mL round-bottomed flask. The resultant mixture was stirred at room-temperature for 2 hours. The reaction mixture was poured into water (20 mL) and extracted with dichloromethane (20 mL×3). The combined organic extracts were dried over anhydrous Na₂SO₄, filtered, and concentrated to dryness under reduced pressure. The resulting residue was purified by FCC (SiO₂, eluent: petroleum ether:ethyl acetate=1:0 to 3:1) to afford the title compound (630 mg, 84%) a white solid. LC-MS (ESI): mass calcd. for C₁₅H₁₆BrFN₂O 338.0 m/z found 338.9 [M+H]⁺.

### Intermediate 114: 3-Bromo-2-(5-fluoropyridin-2-yl)-6,6,7-trimethyl-6,7-dihydro-4H-pyrazolo[5,1-c][1,4]oxazine

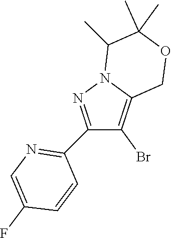 [SMILES: CC1n2nc(-c3ccc(F)cn3)c(Br)c2COC1(C)C]

Step A. 3-(5-(((tert-Butyldimethylsilyl)oxy)methyl)-3-(5-fluoropyridin-2-yl)-1H-pyrazol-1-yl)butan-2-one. 3-Bromobutan-2-one (2.55 g, 16.9 mmol) was added to a solution consisting of 2-(5-(((tert-butyldimethylsilyl)oxy)methyl)-1H-pyrazol-3-yl)-5-fluoropyridine (Intermediate 35, 4.0 g, 13 mmol), Cs₂CO₃ (8.48 g, 26.0 mmol), and CH₃CN (40 mL). The resultant mixture was stirred at room-temperature for 16 hours. The mixture was concentrated to dryness under reduced pressure. The resulting residue was purified by FCC (SiO₂, eluent: petroleum ether:ethyl acetate=1:0 to 3:1) to afford the title compound (4.0 g, 77%) as a clear oil. LC-MS (ESI): mass calcd. for C₁₉H₂₈FN₃O₂Si 377.19 m/z found 378.2 [M+H]⁺.

Step B. 3-(5-(((tert-Butyldimethylsilyl)oxy)methyl)-3-(5-fluoropyridin-2-yl)-1H-pyrazol-1-yl)-2-methylbutan-2-ol. MeLi (29 mL, 1.6 M in Et₂O, 46 mmol) was added dropwise to a −70° C. (dry ice/EtOH) solution consisting of 3-(5-(((tert-butyldimethylsilyl)oxy)methyl)-3-(5-fluoropyridin-2-yl)-1H-pyrazol-1-yl)butan-2-one (3.5 g, 9.3 mmol) and dichloromethane (40 mL). The resultant mixture was stirred for 1 hour. The reaction mixture was gradually warmed to room-temperature and stirred at room-temperature for 12 hours. The reaction mixture was quenched with sat·NH₄Cl (30 mL) and extracted with ethyl acetate (250 mL×3). The combined organic extracts were washed with brine (20 mL), dried over anhydrous Na₂SO₄, filtered, and concentrated to dryness under reduced pressure. The resulting residue was purified by FCC (SiO₂, eluent: petroleum ether:ethyl acetate=1:0 to 10:1) to afford the title compound (1.6 g, 34%) as a yellow oil. LC-MS (ESI): mass calcd. for C₂₀H₃₂FN₃O₂Si 393.22 m/z found 394.5 [M+H]⁺.

Step C. 3-(3-(5-Fluoropyridin-2-yl)-5-(hydroxymethyl)-1H-pyrazol-1-yl)-2-methylbutan-2-ol. TBAF (7.6 mL, 1 M in THF, 7.6 mmol) was added dropwise to a 0° C. (ice/water) solution consisting of 3-(5-(((tert-butyldimethylsilyl)oxy)methyl)-3-(5-fluoropyridin-2-yl)-1H-pyrazol-1-yl)-2-methylbutan-2-ol (1.5 g, 3.8 mmol), and THF (10 mL). The resultant mixture was stirred at room-temperature for 1 hour. The reaction mixture was poured into water (50 mL) and extracted with ethyl acetate (150 mL×3). The combined organic extracts were washed with brine (10 mL×2), dried over anhydrous Na₂SO₄, filtered, and concentrated to dryness under reduced pressure. The resulting residue was purified by FCC (SiO₂, eluent: petroleum ether:ethyl acetate=0:1 to 5:1) to afford the title compound (800 mg, 68%) as a white solid. LC-MS (ESI): mass calcd. for C₁₄H₁₈FN₃O₂ 279.14 m/z found 280.2 [M+H]⁺.

Step D. 2-(5-Fluoropyridin-2-yl)-6,6,7-trimethyl-6,7-dihydro-4H-pyrazolo[5,1-c][1,4]oxazine. 3-(3-(5-Fluoropyridin-2-yl)-5-(hydroxymethyl)-1H-pyrazol-1-yl)-2-methylbutan-2-ol (800 mg, 2.86 mmol) was dissolved in H₃PO₄ (2 mL) and toluene (20 mL) at a 40 mL sealed tube. The resultant mixture was stirred at 130° C. for 16 hours. The reaction mixture was cooled to room-temperature. The reaction mixture was poured into H₂O (30 mL), the pH was adjusted t to pH=8 with 2 N NaOH and extracted with ethyl acetate (150 mL×3). The combined organic extracts were washed with brine (10 mL), dried over anhydrous Na₂SO₄, filtered, and concentrated to dryness under reduced pressure. The resulting residue was purified by FCC (SiO₂, eluent: petroleum ether:ethyl acetate=1:0 to 3:1) to afford the title compound (550 mg, 68%) as a white solid. LC-MS (ESI): mass calcd. for C₁₄H₁₆FN₃O 261.13 m/z found 262.1 [M+H]⁺.

Step E. 3-Bromo-2-(5-fluoropyridin-2-yl)-6,6,7-trimethyl-6,7-dihydro-4H-pyrazolo[5,1-c][1,4]oxazine. The title compound was prepared in a manner analogous to Intermediate 37, Step B, except using 2-(5-fluoropyridin-2-yl)-6,6,7-trimethyl-6,7-dihydro-4H-pyrazolo[5,1-c][1,4]oxazine instead of 2-(5-fluoropyridin-2-yl)-6,7-dihydro-4H-pyrazolo[5,1-c][1,4]oxazine and 3-(5-fluoropyridin-2-yl)-6,7-dihydro-4H-pyrazolo[5,1-c][1,4]oxazine. LC-MS (ESI): mass calcd. for C₁₄H₁₅BrFN₃O 339.04 m/z found 340.1 [M+H]⁺.

### Intermediate 115: 3-Bromo-2-(5-fluoropyridin-2-yl)-6-methyl-6-(trifluoromethyl)-6,7-dihydro-4H-pyrazolo[5,1-c][1,4]oxazine

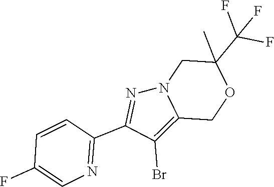 [SMILES: CC1(C(F)(F)F)Cn2nc(-c3ccc(F)cn3)c(Br)c2CO1]

Step A: Ethyl 3-(5-fluoropyridin-2-yl)-1-(3,3,3-trifluoro-2-hydroxy-2-methylpropyl)-1H-pyrazole-5-carboxylate. Ethyl 3-(5-fluoropyridin-2-yl)-1H-pyrazole-5-carboxylate (Intermediate 34, 33 mmol, 1 equiv) was dissolved in DMF, then DBU (36 mmol, 1.1 equiv) was added followed by addition of 2-methyl-2-(trifluoromethyl)oxirane (39 mmol, 1.2 equiv) as a solution in DMF. The flask was sealed and stirred for 15 hours at RT. The reaction was transferred to a separatory funnel and diluted with ethyl acetate and brine. The layers were separated, and the aqueous layer was extracted again with ethyl acetate (100 mL×4). The combined organic layers were washed again with brine, dried over Na₂SO₄, filtered, and concentrate under reduced pressure. The crude oil was purified on silica using hexanes/ethyl acetate 0-50% over 30 min (the product elutes around 7-11% ethyl acetate) to yield the title compound as transparent pale yellow oil. MS (ESI): mass calcd. for C₁₅H₁₅F₄N₃O₃, 361.1; m/z found, 362.1 [M+H]⁺.

Step B: 1,1,1-Trifluoro-3-(3-(5-fluoropyridin-2-yl)-5-(hydroxymethyl)-1H-pyrazol-1-yl)-2-methylpropan-2-ol. Ethyl 3-(5-fluoropyridin-2-yl)-1-(3,3,3-trifluoro-2-hydroxy-2-methylpropyl)-1H-pyrazole-5-carboxylate (9.7 mmol, 1 equiv) was dissolved in THF (180 mL), and cooled to −12° C. in an ice/MeOH bath under N₂ atm. The reaction mixture was stirred for 15 min, then lithium aluminum hydride (1M in THF, 14.4 mmol, 1.5 equiv) was added dropwise over 15 min at −10° C., the reaction was allowed to slowly warm to 0° C. over 2 hours. The reaction was then diluted with ethyl acetate (20 mL) at 0° C. and stirred for 1 hour while warming to RT. The reaction was then quenched with saturated aqueous Rochelle's salt (15 mL) and then stirred vigorously for 2 hours before being transferred to a separatory funnel. The layers were allowed to separate, and the aqueous layer was extracted with more ethyl acetate (70 mL×3). The combined organic layers were dried over Na₂SO₄, filtered, and concentrated under reduced pressure to yield a clear colorless oil. MS (ESI): mass calcd. for C₁₃H₁₃F₄N₃O₂, 319.0; m/z found, 320.0 [M+H]⁺.

Step C: 3-Bromo-2-(5-fluoropyridin-2-yl)-6-methyl-6-(trifluoromethyl)-6,7-dihydro-4H-pyrazolo[5,1-c][1,4]oxazine. The title compound was prepared in a manner analogous to Intermediate 81 Step C-D, except using 1,1,1-trifluoro-3-(3-(5-fluoropyridin-2-yl)-5-(hydroxymethyl)-1H-pyrazol-1-yl)-2-methylpropan-2-ol instead of 1-fluoro-3-(3-(4-fluorophenyl)-5-(hydroxymethyl)-1H-pyrazol-1-yl)propan-2-ol (Intermediate 81, Step B) in Step C. MS (ESI): mass calcd. for C₁₃H₁₀BrF₄N₃O, 379.0; m/z found, 380.0[M+H]⁺.

### Intermediate 116: 3-Bromo-2-(4-fluorophenyl)-6,7-dihydro-4H-pyrazolo[5,1-c][1,4]oxazin-4-one

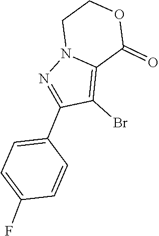 [SMILES: O=C1OCCn2nc(-c3ccc(F)cc3)c(Br)c21]

Step A: Ethyl 1-(2-((tert-butyldimethylsilyl)oxy)ethyl)-3-(4-fluorophenyl)-1H-pyrazole-5-carboxylate. (2-Bromoethoxy)(tert-butyl)dimethylsilane (0.221 mL, 1.03 mmol) was added to a solution consisting of ethyl 3-(4-fluorophenyl)-1H-pyrazole-5-carboxylate (Intermediate 33, 200 mg, 0.854 mmol), Cs₂CO₃ (835 mg, 2.56 mmol), and DMA (3 mL). The resulting mixture was stirred for 3 hours at room-temperature. The reaction mixture was poured into NH₄Cl (5 mL) and extracted with ethyl acetate (15 mL×3). The combined organic extracts were dried over anhydrous Na₂SO₄, filtered, and concentrated to dryness under reduced pressure. The resulting residue was purified by FCC (SiO₂, eluent: petroleum ether:ethyl acetate=1:0 to 10:1) to afford the title compound (110 mg, 31% yield) as a white solid. MS (ESI): mass calcd. for C₂₀H₂₉FN₂O₃Si 392.2; m/z found 393.2 [M+H]⁺.

Step B: Ethyl 1-(2-acetoxyethyl)-4-bromo-3-(4-fluorophenyl)-1H-pyrazole-5-carboxylate. Ethyl 1-(2-((tert-butyldimethylsilyl)oxy)ethyl)-3-(4-fluorophenyl)-1H-pyrazole-5-carboxylate (1.00 g, 2.55 mmol) and NBS (680 mg, 3.82 mmol) in HOAc (10 mL) were added to a 20 mL microwave tube. The resulting mixture was heated at 150° C. via microwave irradiation for 0.1 hours. The reaction mixture was cooled to room-temperature. The mixture was concentrated to dryness under reduced pressure. The resulting product was poured into sat aq·NaHCO₃(5 mL) and extracted with ethyl acetate (15 mL×3). The combined organic extracts were dried over anhydrous Na₂SO₄, filtered, and concentrated to dryness under reduced pressure. The resulting residue was purified by FCC (SiO₂, eluent: petroleum ether:ethyl acetate=1:0 to 1:3) to afford the title compound (727 mg, 90.65% purity, 32.97%) as a white solid. MS (ESI): mass calcd. for C₁₆H₁₆BrFN₂O₄ 399.2; m/z found 401.2 [M+H]⁺.

Step C: 4-Bromo-3-(4-fluorophenyl)-1-(2-hydroxyethyl)-1H-pyrazole-5-carboxylic acid. Lithium hydroxide monohydrate (114 mg, 2.71 mmol) was added to a solution of ethyl 1-(2-acetoxyethyl)-4-bromo-3-(4-fluorophenyl)-1H-pyrazole-5-carboxylate (720 mg, 1.80 mmol) in EtOH (8 mL) and H₂O (4 mL). The mixture was stirred at 65° C. for 3 hours. The reaction mixture was concentrated under reduced pressure, the reaction mixture was adjusted pH=4 with saturated 2 M HCl (1 mL) and extracted with ethyl acetate (10×3 mL). The organic extract was dried over anhydrous Na₂SO₄, filtered, and evaporated to dryness under reduced pressure to give the title product (486 mg, 98.32% purity) as a white solid. MS (ESI): mass calcd. for C₁₂H₁₀BrFN₂O₃ 329.1; m/z found 330.7 [M+H]⁺.

Step D: 3-Bromo-2-(4-fluorophenyl)-6,7-dihydro-4H-pyrazolo[5,1-c][1,4]oxazin-4-one. 4-Bromo-3-(4-fluorophenyl)-1-(2-hydroxyethyl)-1H-pyrazole-5-carboxylic acid (380 mg, 1.16 mmol), was dissolved in DMF (5 mL), then TEA (0.483 mL, 3.46 mmol) and T3P® (1.08 mL, 1.73 mmol) were added to the above solution. The solution was stirred at room-temperature for 16 hours. It was poured into sat aq. NH₄Cl (5 mL) and extracted with ethyl acetate (15 mL×3). The organic layer was evaporated and the resulting residue was purified by FCC (SiO₂, eluent: petroleum ether:ethyl acetate=10:1 to 1:1) to afford the title compound (300 mg, 98.25% purity, 82% yield) as a white solid. MS (ESI): mass calcd. for C₁₂H₈BrFN₂O₂ 310.0; m/z found 310.8 [M+H]⁺.

### Intermediate 117: 3-Bromo-2-(5-fluoropyridin-2-yl)-6,7-dihydro-4H-pyrazolo[5,1-c][1,4]oxazin-4-one

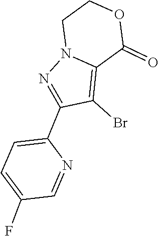 [SMILES: O=C1OCCn2nc(-c3ccc(F)cn3)c(Br)c21]

The title compound was prepared in a manner analogous to Intermediate 116, Steps A-B, except using ethyl 3-(5-fluoropyridin-2-yl)-1H-pyrazole-5-carboxylate (Intermediate 34) instead of ethyl 3-(4-fluorophenyl)-1H-pyrazole-5-carboxylate (Intermediate 33) and DMF instead of DMA in Step A. MS (ESI): mass calcd. or C₁₁H₇BrFN₃O₂ 311.0; m/z found 312.0 [M+H]⁺.

### Intermediate 118: 3-Bromo-2-(4-fluorophenyl)-6,7-dihydro-5H-pyrazolo[5,1-b][1,3]oxazine

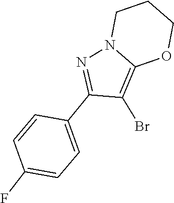 [SMILES: Fc1ccc(-c2nn3c(c2Br)OCCC3)cc1]

The title compound was prepared in a manner analogous to Intermediate 119, Step D-E except using 5-(4-fluorophenyl)-1,2-dihydro-3H-pyrazol-3-one (Intermediate 127, Step A) instead of 5-(5-fluoropyridin-2-yl)-1,2-dihydro-3H-pyrazol-3-one in Step D. MS (ESI): mass calcd. for C₁₂H₁₀BrFN₂O 296.0; m/z found 297.0 [M+H]⁺.

### Intermediate 119: 3-Bromo-2-(5-fluoropyridin-2-yl)-6,7-dihydro-5H-pyrazolo[5,1-b][1,3]oxazine

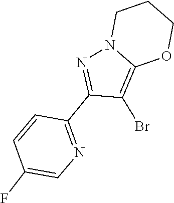 [SMILES: Fc1ccc(-c2nn3c(c2Br)OCCC3)nc1]

Step A: 1-(5-Fluoropyridin-2-yl)ethan-1-one. MeMgBr (6.6 mL, 3 M in 2-Me-THF, 19.7 mmol) was added dropwise to a cooled (−65° C.; dry ice/ethanol) solution consisting of 5-fluoropicolinonitrile (2 g, 16.4 mmol) and THF (20 mL). The resultant mixture was stirred at −65° C. (dry ice/ethanol) for 2 hours and then stirred at room-temperature for 2 hours. The reaction mixture was quenched with sat. NH₄Cl (10 mL) and extracted with ethyl acetate (100 mL×3). The combined organic extracts were washed with brine (100 mL), dried over anhydrous Na₂SO₄, filtered and concentrated to dryness under reduced pressure. The resulting residue was purified by FCC (SiO₂, eluent: petroleum ether:ethyl acetate 0-10%) to afford the title compound (1.9 g, 83%) as yellow oil. ¹H NMR (400 MHz, CDCl₃): δ=8.49 (d, J=2.8 Hz, 1H), 8.10 (dd, J=4.8, 8.8 Hz, 1H), 7.50 (dt, J=2.9, 8.3 Hz, 1H), 2.75-2.64 (m, 3H).

Step B: Methyl 3-(5-fluoropyridin-2-yl)-3-oxopropanoate. Sodium hydride in mineral oil (1.1 g, 27.3 mmol) was added in portions to a 0° C. (ice/water) solution consisting of 1-(5-fluoropyridin-2-yl)ethan-1-one (1.9 g, 13.7 mmol) and dimethyl carbonate (120 mL). The resultant mixture was stirred at stirred for 3 hours. The reaction mixture was cooled to room-temperature, quenched with sat. NH₄Cl (50 mL) at 0° C. (ice/water), and extracted with ethyl acetate (2×200 mL). The combined organic extracts were washed with brine (100 mL), dried over Na₂SO₄, filtered, and concentrated under reduce pressure. The resulting residue was purified by FCC (SiO₂, 0-20% ethyl acetate:petroleum ether:) to afford the title compound (1.4 g, 52%) as yellow oil. ¹H NMR (400 MHz, CDCl₃): δ 8.48 (d, J=2.8 Hz, 1H), 8.14 (d, J=4.5 Hz, 1H), 7.96 (dd, J=4.5, 8.8 Hz, 1H), 3.83 (s, 2H), 3.74 (s, 3H).

Step C: 5-(5-Fluoropyridin-2-yl)-1,2-dihydro-3H-pyrazol-3-one. Hydrazine hydrate (1.1 g, 21.3 mmol) was added to a mixture consisting of methyl 3-(5-fluoropyridin-2-yl)-3-oxopropanoate (1.4 g, 7.1 mmol) and acetic acid (14 mL). The resultant mixture was stirred at 80° C. for 24 hours. The reaction mixture was cooled to room-temperature and concentrated under reduce pressure. The reaction solution was concentrated and filtered. The filter cake was washed with water (15 mL) before drying under reduced pressure to afford the title compound (0.9 g, 70.7%) as a yellow solid. ¹H NMR (400 MHz, DMSO-d₆) δ 12.22 (br s, 1H), 9.91 (br s, 1H), 8.56 (d, J=2.8 Hz, 1H), 7.88-7.74 (m, 2H), 6.02 (s, 1H).

Step D: 2-(5-Fluoropyridin-2-yl)-6,7-dihydro-5H-pyrazolo[5,1-b][1,3]oxazine. A solution of 5-(5-fluoropyridin-2-yl)-1,2-dihydro-3H-pyrazol-3-one (680 mg, 3.80 mmol), 1-bromo-3-chloropropane (717 mg, 4.55 mmol), K₂CO₃ (2.1 g, 15.12 mmol), in CH₃CN (20 mL) was stirred at 90° C. for 4 hours. The reaction mixture was cooled to room-temperature. The mixture was concentrated under reduce pressure. The resulting residue was purified by FCC (SiO₂, eluent: petroleum ether:ethyl acetate=3:1 to 1:2) to afford 3-(5-fluoropyridin-2-yl)-1,4,5,6-tetrahydropyrano[2,3-c]pyrazole (undesired, 50 mg, 6%) and the title compound (180 mg, 21.6%). ¹H NMR (400 MHz, CDCl₃): δ 8.45 (d, J=2.8 Hz, 1H), 7.85 (dd, J=4.4, 8.8 Hz, 1H), 7.40 (dt, J=2.8, 8.4 Hz, 1H), 6.05 (s, 1H), 4.34-4.30 (m, 2H), 4.24 (t, J=6.4 Hz, 2H), 2.35-2.24 (m, 2H); 19F NMR (376 MHz, CDCl₃): −128.82 (br s, 1F).

Step E: 3-Bromo-2-(5-fluoropyridin-2-yl)-6,7-dihydro-5H-pyrazolo[5,1-b][1,3]oxazine. NBS (160.8 mg, 0.9 mmol) was added to a solution consisting of 2-(5-fluoropyridin-2-yl)-6,7-dihydro-5H-pyrazolo[5,1-b][1,3]oxazine (180 mg, 0.82 mmol) and dichloromethane (5 mL). The resultant mixture was stirred at room-temperature for 30 minutes. The reaction mixture was quenched with sat. NaHCO₃(80 mL) and extracted with dichloromethane (2×50 mL). The organic layers were combined and washed with brine (50 mL), dried over anhydrous Na₂SO₄, filtered, and concentrated under reduce pressure. The resulting residue was purified by FCC (SiO₂, eluent: petroleum ether:ethyl acetate=3:1 to 1:2) to afford the title compound (180 mg, 73.5% yield) as a white solid. ¹H NMR (400 MHz, CDCl₃): δ 8.56 (d, J=2.8 Hz, 1H), 7.99 (dd, J=4.4, 8.8 Hz, 1H), 7.46 (dt, J=3.2, 8.4 Hz, 1H), 4.44-4.39 (m, 2H), 4.26 (t, J=6.4 Hz, 2H), 2.36-2.29 (m, 2H).

### Intermediate 120: 3-Bromo-2-(3,5-difluoropyridin-2-yl)-6,7-dihydro-5H-pyrazolo[5,1-b][1,3]oxazine

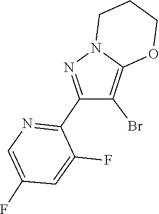 [SMILES: Fc1cnc(-c2nn3c(c2Br)OCCC3)c(F)c1]

Step A: 3,5-Difluoropicolinic acid. A solution of 3,5-difluoropicolinonitrile (2.6 g, 18.6 mmol) in H₂SO₄ (18 mL) and water (2 mL) was stirred and heated at 110° C. for 22 h. The reaction was cooled to room temperature and then poured into ice water (50 mL). The resulting precipitate was collected via filtration, washed with water (20 mL) and dried under high vacuum to afford the title compound (2.5 g, 85%). ¹H NMR (400 MHz, DMSO-d₆) 8.62 (d, J=2.4 Hz, 1H), 8.11 (ddd, J=2.2, 8.9, 10.7 Hz, 1H).

Step B: Methyl 3,5-difluoropicolinate. A solution of 3,5-difluoropicolinic acid (1.9 g, 11.9 mmol) in toluene (16 mL) and MeOH (4 mL) was added to (diazomethyl)trimethylsilane (8.96 mL, 17.9 mmol) at room temperature and the resulting mixture was stirred for 2 h. The reaction was quenched with aqueous NH₄Cl and extracted with EtOAc (100 mL). The combined organics layers were washed with brine, dried over Na₂SO₄, filtered, and concentrated under reduced pressure. The resulting residue was purified by FCC (eluent: petroleum ether:ethyl acetate=1:0 to 3:1) to afford the title compound (1.67 g, 79%) as a colorless solid. MS (ESI): mass calcd. for C₇H₅F₂NO₂ 173.0; m/z found 173.9 [M+H]⁺. ¹H NMR (400 MHz, CDCl₃): δ 8.45 (d, J=2.3 Hz, 1H), 7.34 (ddd, J=2.3, 7.7, 9.9 Hz, 1H), 4.01 (s, 3H).

Step C: Ethyl 3-(3,5-difluoropyridin-2-yl)-3-oxopropanoate. To a solution of methyl 3,5-difluoropicolinate (1.1 g, 6.35 mmol) in ethyl acetate (15 mL) was added potassium t-pentoxide (7.62 mL, 7.26 mmol) at −10° C. The reaction mixture was warmed to room temperature and stirred for 12 h. The reaction mixture was quenched with sat NH₄Cl (30 mL). The aqueous layer was extracted with EtOAc (40 mL) and the organics layer was dried over MgSO₄, filtered, and concentrated under reduced pressure. The resulting residue was purified by FCC (eluent: petroleum ether:ethyl acetate=1:0 to 3:1) to afford the title compound (450 mg, 30%) as a colorless oil.

Step D: 3-Bromo-2-(3,5-difluoropyridin-2-yl)-6,7-dihydro-5H-pyrazolo[5,1-b][1,3]oxazine. The title compound was prepared in a manner analogous to Intermediate 119, Step C-E except using ethyl 3-(3,5-difluoropyridin-2-yl)-3-oxopropanoate instead of methyl 3-(5-fluoropyridin-2-yl)-3-oxopropanoate (Intermediate 119, Step B) in Step C. MS (ESI): mass calcd. for C₁₁H₈BrF₂N₃O 315.0; m/z found 315.9[M+H]⁺. ¹H NMR (400 MHz, CDCl₃): δ 8.47 (d, J=2.0 Hz, 1H), 7.32 (ddd, J=2.4, 8, 9.2 Hz, 1H), 4.46-4.41 (m, 2H), 4.28 (t, J=6.4 Hz, 2H), 2.39-2.30 (m, 2H).

### Intermediate 121: 3-Bromo-2-(4-fluorophenyl)-6-methyl-6,7-dihydro-5H-pyrazolo[5,1-b][1,3]oxazine

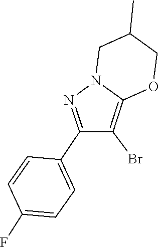 [SMILES: CC1COc2c(Br)c(-c3ccc(F)cc3)nn2C1]

The title compound was made in a manner analogous to Intermediate 127, step A-C except using 1-bromo-3-chloro-2-methylpropane instead of 1,3-dichloro-2,2-dimethylpropane, ACN instead of NMP and K₂CO₃ instead of NaH in Step B. In addition, Step B was heated at 90° C. for 4 h using conventional heating rather than microwave irradiation and KI was not added. MS (ESI): mass calcd. for C₁₃H₁₂BrFN₂O, 310.0; m/z found, 311.0 [M+H]⁺.

### Intermediate 122: 3-Bromo-2-(5-fluoropyridin-2-yl)-6-methyl-6,7-dihydro-5H-pyrazolo[5,1-b][1,3]oxazine-methane (1/1)

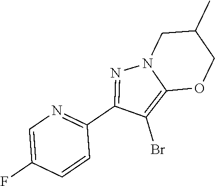 [SMILES: CC1COc2c(Br)c(-c3ccc(F)cn3)nn2C1]

The title compound was prepared in a manner analogous the Intermediate 119, Steps A-E except using 1-bromo-3-chloro-2-methylpropane instead of 1-bromo-3-chloropropane in Step D. ¹H NMR (400 MHz, CDCl₃): δ 8.57 (d, J=2.8 Hz, 1H), 8.00 (dd, J=4.4, 8.8 Hz, 1H), 7.49-7.44 (m, 1H), 4.44-4.29 (m, 2H), 4.02-3.94 (m, 1H), 3.82 (dd, J=9.2, 12.4 Hz, 1H), 2.56-2.51 (m, 1H), 1.18 (d, J=6.8 Hz, 3H).

### Intermediate 123: 3-Bromo-2-(5-fluoropyridin-2-yl)-5-methyl-6,7-dihydro-5H-pyrazolo[5,1-b][1,3]oxazine

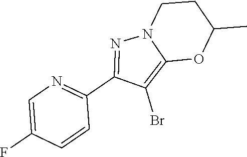 [SMILES: CC1CCn2nc(-c3ccc(F)cn3)c(Br)c2O1]

The title compound was prepared in a manner analogous the Intermediate 119, Steps A-E except using 1,3-dibromobutane instead of 1-bromo-3-chloropropane in Step D. ¹H NMR (400 MHz, CDCl₃): δ 8.56 (br s, 1H), 7.99 (br dd, J=4.4, 8.4 Hz, 1H), 7.48-7.41 (m, 1H), 4.51-4.43 (m, 1H), 4.34-4.27 (m, 1H), 4.23-4.15 (m, 1H), 2.28-2.20 (m, 1H), 2.19-2.06 (m, 1H), 1.55 (d, J=6.4 Hz, 3H).

### Intermediate 124: cis-3-Bromo-2-(5-fluoropyridin-2-yl)-5,7-dimethyl-6,7-dihydro-5H-pyrazolo[5,1-b][1,3]oxazine

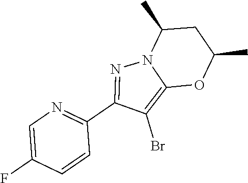 [SMILES: C[C@@H]1C[C@H](C)n2nc(-c3ccc(F)cn3)c(Br)c2O1]

Step A: cis-2-(5-Fluoropyridin-2-yl)-5,7-dimethyl-6,7-dihydro-5H-pyrazolo[5,1-b][1,3]oxazine. The title compound was prepared in a manner analogous to Intermediate 119, Step A-D except using 2,4-dibromopentane instead of 1-bromo-3-chloropropane in Step D. ¹H NMR (400 MHz, CDCl₃): δ 8.46 (d, J=2.8 Hz, 1H), 7.91 (dd, J=4.4, 8.8 Hz, 1H), 7.41 (dt, J=3.2, 8.4 Hz, 1H), 6.04 (s, 1H), 4.41-4.25 (m, 2H), 2.24 (ddd, J=1.6, 5.2, 14.0 Hz, 1H), 1.85 (td, J=11.6, 14.0 Hz, 1H), 1.66 (d, J=6.4 Hz, 3H), 1.48 (d, J=6.4 Hz, 3H).

Step B: cis-3-Bromo-2-(5-fluoropyridin-2-yl)-5,7-dimethyl-6,7-dihydro-5H-pyrazolo[5,1-b][1,3]oxazine. cis-2-(5-Fluoropyridin-2-yl)-5,7-dimethyl-6,7-dihydro-5H-pyrazolo[5,1-b][1,3]oxazine (600 mg, 2.43 mmol, racemic), NBS (518 mg, 2.91 mmol), and dichloromethane (10 mL) were added to a 100 mL round-bottomed flask. The resultant mixture was stirred at 25° C. for 10 minutes. The reaction mixture was quenched with the sat. NaHCO₃(100 mL) and extracted with ethyl acetate (30 mL×2). The combined organic extracts were washed with brine (20 mL×4), dried over anhydrous Na₂SO₄, filtered, and concentrated to dryness under reduced pressure. The resulting residue was purified by FCC (eluent: petroleum ether:ethyl acetate=1:0 to 2:1) to afford the product (514 mg, 65%) as a yellow solid. The product was combined with additional batches (247 mg) of the title compound and suspended in water (10 mL), the mixture frozen using dry ice/ethanol, and then lyophilized to dryness to afford the title compound (700.6 mg) as a yellow solid. LC-MS (ESI): mass calcd. for C₁₃H₁₃BrFN₃O 325.02 m/z, found 325.9 [M+H]⁺. ¹H NMR (400 MHz, CDCl₃): δ 8.57 (d, J=3.2 Hz, 1H), 7.98 (dd, J=4.4, 8.8 Hz, 1H), 7.46 (dt, J=3.2, 8.4 Hz, 1H), 4.49-4.27 (m, 2H), 2.25-2.31 (m, 1H), 1.88 (td, J=11.6, 14.2 Hz, 1H), 1.66 (d, J=6.4 Hz, 3H), 1.55 (d, J=6.4 Hz, 3H).

### Intermediate 125: trans-3-Bromo-2-(5-fluoropyridin-2-yl)-5,7-dimethyl-6,7-dihydro-5H-pyrazolo[5,1-b][1,3]oxazine

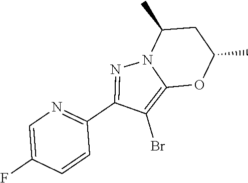 [SMILES: C[C@H]1C[C@H](C)n2nc(-c3ccc(F)cn3)c(Br)c2O1]

Step A: trans-2-(5-Fluoropyridin-2-yl)-5,7-dimethyl-6,7-dihydro-5H-pyrazolo[5,1-b][1,3]oxazine. The title compound was prepared in a manner analogous to Intermediate 119, Step A-D except using 2,4-dibromopentane instead of 1-bromo-3-chloropropane in Step D. ¹H NMR (400 MHz, CDCl₃): δ 8.46 (d, J=2.8 Hz, 1H), 7.93-7.82 (m, 1H), 7.41 (dt, J=3.2, 8.4 Hz, 1H), 5.99 (s, 1H), 4.58-4.46 (m, 2H), 2.28-2.15 (m, 1H), 1.95 (td, J=2.4, 14.0 Hz, 1H), 1.62 (d, J=6.8 Hz, 3H), 1.48 (d, J=6.4 Hz, 3H).

Step B: trans-3-Bromo-2-(5-fluoropyridin-2-yl)-5,7-dimethyl-6,7-dihydro-5H-pyrazolo[5,1-b][1,3]oxazine. trans-2-(5-Fluoropyridin-2-yl)-5,7-dimethyl-6,7-dihydro-5H-pyrazolo[5,1-b][1,3]oxazine (200 mg, 0.809 mmol, racemic), NBS (173 mg, 0.971 mmol), and dichloromethane (4 mL) were added to a 50 mL round-bottomed flask. The resultant mixture was stirred at 25° C. for 10 minutes. The reaction mixture was quenched with sat·NaHCO₃(50 mL) and extracted with ethyl acetate (20 mL×2). The combined organic extracts were washed with brine (20 mL×4), dried over anhydrous Na₂SO₄, filtered, and concentrated to dryness under reduced pressure. The resulting residue was purified by FCC (eluent: petroleum ether:ethyl acetate=1:0 to 2:1) to afford the product (145 mg, 55%) as a yellow solid. The product was combined with additional batches (112 mg) of the title compound and suspended in water (10 mL), the mixture frozen using dry ice/ethanol, and then lyophilized to dryness to afford the title compound (245 mg) as a yellow solid. LC-MS (ESI): mass calcd. for C₁₃H₁₃BrFN₃O 325.02 m/z, found 325.9 [M+H]⁺. ¹H NMR (400 MHz, CDCl₃): δ 8.57 (d, J=2.8 Hz, 1H), 7.99 (dd, J=4.4, 8.8 Hz, 1H), 7.46 (dt, J=2.8, 8.4 Hz, 1H), 4.67-4.48 (m, 2H), 2.29-2.18 (m, 1H), 1.99 (td, J=2.8, 14.4 Hz, 1H), 1.63 (s, 3H), 1.54 (d, J=6.4 Hz, 3H).

### Intermediate 126: 3′-Bromo-2′-(4-fluorophenyl)-5′H,7′H-spiro[oxetane-3,6′-pyrazolo[5,1-b][1,3]oxazine]

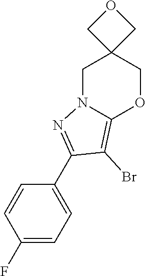 [SMILES: Fc1ccc(-c2nn3c(c2Br)OCC2(COC2)C3)cc1]

The title compound was prepared in a manner analogous to Intermediate 119, Step D-E except using 5-(4-fluorophenyl)-1,2-dihydro-3H-pyrazol-3-one (Intermediate 127, Step A) instead of 5-(5-fluoropyridin-2-yl)-1,2-dihydro-3H-pyrazol-3-one, and 3,3-bis(chloromethyl)oxetane instead of 1-bromo-3-chloropropane in Step D. MS (ESI): mass calcd. for C₁₄H₁₂BrFN₂O₂ 338.0; m/z found 339.0 [M+H]⁺.

### Intermediate 127: 3-Bromo-2-(4-fluorophenyl)-6,6-dimethyl-6,7-dihydro-5H-pyrazolo[5,1-b][1,3]oxazine

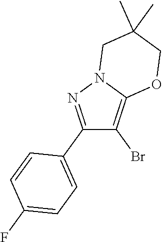 [SMILES: CC1(C)COc2c(Br)c(-c3ccc(F)cc3)nn2C1]

Step A: 5-(4-Fluorophenyl)-1,2-dihydro-3H-pyrazol-3-one. Hydrazine (3 mL, 93.7 mmol) was added to a solution of methyl 4-fluorobenzoylacetate (10.1 g, 51.5 mmol) in AcOH (20 mL) and the resulting mixture was heated to 80° C. for 24 h at which time an additional 5 mL of hydrazine was added to the reaction. After heating for an additional 18 h, the reaction was cooled to room temperature and diluted with Et₂₀ (20 mL) and cooled to 0° C. in an ice bath. The resulting solids were collected via filtration and dried under high vacuum to obtain the title compound (5.47 g, 60%). MS (ESI): mass calcd. for C₉H₇FN₂O, 178.1; m/z found, 179.0 [M+H]⁺.

Step B. 2-(4-Fluorophenyl)-6,6-dimethyl-6,7-dihydro-5H-pyrazolo[5,1-b][1,3]oxazine. 5-(4-Fluorophenyl)-1,2-dihydro-3H-pyrazol-3-one (893.5 mg, 4.251 mmol), potassium iodide (95.3 mg, 0.574 mmol), 1,3-dichloro-2,2-dimethylpropane (690 mg, 14.89 mmol), NMP (9 mL), was add to 2 mL microwave vial, followed by NaH (60% dispersion in mineral oil, 346.8 mg, 8.671 mmol). The mixture was allowed to stir without a cap for 2 min until bubbling subsided. Once all the reactants were homogeneously mixed the vial was capped and placed in a microwave reactor for 2 h at 180° C. The crude reaction was diluted with EtOAc (−50 mL), washed with 0.1M HCl (−25 mL×2), with 5% LiCl solution (−25 mL×1), dried over MgSO₄, filtered, and concentrated under reduced pressure to afford a brown-yellow solid. Purification (FCC, SiO₂, hex to 100% EtOAc) afforded the title compound (767 mg, 73%). MS (ESI): mass calcd. for C₁₄H₁₅FN₂O, 246.3; m/z found, 247.2[M+H]⁺.

Step C. 3-Bromo-2-(4-fluorophenyl)-6,6-dimethyl-6,7-dihydro-5H-pyrazolo[5,1-b][1,3]oxazine. The title compound was prepared in a manner analogous to Intermediate 37, Step B, except using 2-(4-fluorophenyl)-6,6-dimethyl-6,7-dihydro-5H-pyrazolo[5,1-b][1,3]oxazine instead of 2-(5-fluoropyridin-2-yl)-6,7-dihydro-4H-pyrazolo[5,1-c][1,4]oxazine and 3-(5-fluoropyridin-2-yl)-6,7-dihydro-4H-pyrazolo[5,1-c][1,4]oxazine. MS (ESI): mass calcd. for C₁₄H₁₄BrFN₂O, 324.0; m/z found, 325.1 [M+H]⁺.

### Intermediate 128: 3-Bromo-2-(5-fluoro-2-pyridyl)-6,6-dimethyl-5,7-dihydropyrazolo[5,1-b][1,3]oxazine

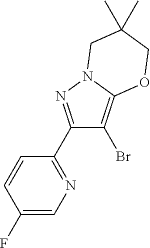 [SMILES: CC1(C)COc2c(Br)c(-c3ccc(F)cn3)nn2C1]

The title compound was made analogous to Intermediate 119, Steps A-E except using 1,3-dibromo-2,2-dimethylpropane instead of 1-bromo-3-chloropropane, and DMF instead of ACN in Step D. MS (ESI): mass calcd. for C₁₃H₁₃BrFN₃O, 325.0; m/z found, 326.0 [M+H]⁺. ¹H NMR (400 MHz, CDCl₃): δ 8.56 (br d, J=2.8 Hz, 1H), 8.00 (dd, J=4.4, 8.8 Hz, 1H), 7.49-7.42 (m, 1H), 4.01 (s, 2H), 3.93 (s, 2H), 1.17 (s, 6H).

### Intermediate 129: 3-Bromo-2-(4-fluorophenyl)-5,6,7,8-tetrahydropyrazolo[5,1-b][1,3]oxazepane

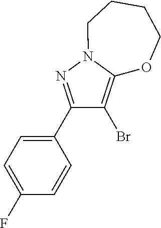 [SMILES: Fc1ccc(-c2nn3c(c2Br)OCCCC3)cc1]

The title compound was prepared in a manner analogous to Intermediate 127, Step A-C, except using 1,4-dibromobutane instead of 1,3-dichloro-2,2-dimethylpropane in Step B, and KI was not added in Step B. MS (ESI): mass calcd. for C₁₃H₁₂BrFN₂O, 310.1; m/z found, 311.0 [M+H]⁺.

### Intermediate 130: 3-Bromo-2-(5-fluoropyridin-2-yl)-5,6,7,8-tetrahydropyrazolo[5,1-b][1,3]oxazepane

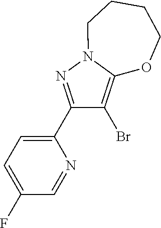 [SMILES: Fc1ccc(-c2nn3c(c2Br)OCCCC3)nc1]

The title compound was made analogous to Intermediate 119, Steps A-E except using 1,4-dibromobutane instead of 1-bromo-3-chloropropane in Step D. MS (ESI): mass calcd. for C₁₂H₁₁BrFN₃O, 311.1; m/z found, 312.0 [M+H]⁺. ¹H NMR (400 MHz, CDCl₃): δ 8.56 (d, J=2.8 Hz, 1H), 7.98 (dd, J=4.4, 8.8 Hz, 1H), 7.46 (dt, J=2.8, 8.4 Hz, 1H), 4.36-4.31 (m, 2H), 4.20-4.15 (m, 2H), 2.15-2.07 (m, 2H), 1.96-1.89 (m, 2H).

### Intermediate 131: Benzyl 3-bromo-2-(5-fluoropyridin-2-yl)-6,7-dihydropyrazolo[1,5-a]pyrazine-5(4H)-carboxylate

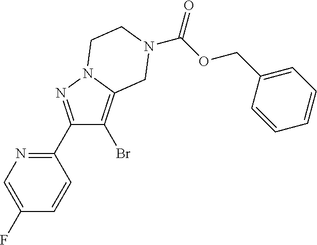 [SMILES: O=C(OCc1ccccc1)N1CCn2nc(-c3ccc(F)cn3)c(Br)c2C1]

The title compound was prepared in a manner analogous to Intermediate 37, Steps A-B, except using 5-((benzyloxy)carbonyl)-4,5,6,7-tetrahydro-[1,2,3]oxadiazolo[3,4-a]pyrazin-8-ium-3-olate (Intermediate 18) instead of 6,7-dihydro-4H-[1,2,3]oxadiazolo[4,3-c][1,4]oxazin-8-ium-3-olate (Intermediate 2) in Step A. MS (ESI): mass calcd. for C₁₉H₁₆BrFN₄O₂, 430.0; m/z found, 431.0 [M+H]⁺. ¹H NMR (400 MHz, CDCl₃): δ 8.54 (d, J=3.0 Hz, 1H), 7.95 (dd, J=8.8, 4.4 Hz, 1H), 7.47-7.40 (m, 1H), 7.40-7.29 (m, 5H), 5.19 (s, 2H), 4.65 (s, 2H), 4.22 (s, 2H), 4.00-3.91 (m, 2H).

### Intermediate 132: 1-Benzyl-4-bromo-6-cyclopropyl-1H-pyrazolo[3,4-b]pyridine

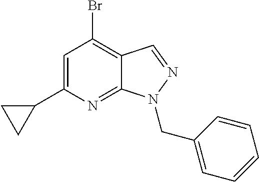 [SMILES: Brc1cc(C2CC2)nc2c1cnn2Cc1ccccc1]

Step A. 1-Benzyl-6-cyclopropyl-1H-pyrazolo[3,4-b]pyridin-4-ol. To a solution of 1-benzyl-1H-pyrazol-5-amine (2 g, 11 mmol) and ethyl 3-cyclopropyl-3-oxopropanoate (1.7 g, 11 mmol) in toluene (15 mL) was added acetic acid (0.063 mL, 1.1 mmol). The reaction mixture was heated to reflux for 10 h under a Dean-Stark trap. The mixture was cooled to room temperature and concentrated. The residue was dissolved in 3 mL of phenyl ether-biphenyl eutectic and added dropwise to 3 mL of phenyl ether-biphenyl eutectic at 275° C. The reaction was maintained at this temperature for 2 h then cooled to room temperature. Purification by chromatography (silica gel, 0-100% EtOAc/hexanes) afforded 821 mg (28%) of the title compound. MS (ESI): mass calcd. for C₁₆H₁₅N₃O, 265.1; m/z found, 266.2 [M+H]⁺.

Step B. 1-Benzyl-4-bromo-6-cyclopropyl-1H-pyrazolo[3,4-b]pyridine. A suspension of 1-benzyl-6-cyclopropyl-1H-pyrazolo[3,4-b]pyridin-4-ol (800 mg, 3 mmol) and phosphorus oxybromide (1.3 g, 4.6 mmol) in toluene was heated to 115° C. then dimethylformamide (2.4 mL, 30 mmol) was added slowly over 1 h. The reaction was cooled to room temperature then quenched with saturated aqueous NaHCO₃. The resulting mixture was extracted with EtOAc (3×). The combined organic extracts were washed with H₂O and brine then dried (Na₂SO₄), filtered, and concentrated. Purification by chromatography (silica gel, 0-100% EtOAc/hexanes) afforded 234 mg (23%) of the title compound. MS (ESI): mass calcd. for C₁₆H₁₄BrN₃, 327.0; m/z found, 328.0 [M+H]⁺. ¹H NMR (400 MHz, CDCl₃): δ 7.91 (s, 1H), 7.38-7.25 (m, 5H), 7.21 (s, 1H), 5.59 (s, 2H), 2.11 (tt, J=8.07, 4.75 Hz, 1H), 1.23-1.14 (m, 2H), 1.12-1.04 (m, 2H).

### Intermediate 134: 3-Chloro-2-fluoro-4-((5-fluoropyridin-2-yl)ethynyl)pyridine

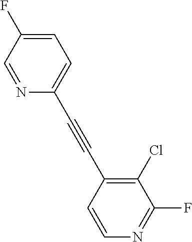 [SMILES: Fc1ccc(C#Cc2ccnc(F)c2Cl)nc1]

To a vial containing 4-bromo-3-chloro-2-fluoropyridine (105 mg, 0.5 mmol), 2-ethynyl-5-fluoropyridine (91 mg, 0.75 mmol), Cu(I) iodide (4.8 mg, 0.025 mmol), and [1,1′-bis(diphenylphosphino)ferrocene]dichloropalladium(II) (21 mg, 0.025 mmol) was added triethylamine (0.7 mL, 5 mmol). The vial was capped and the reaction mixture was degassed under vacuum then refilled with N₂. The mixture was heated to 90° C. for 4 h. The reaction mixture was cooled, diluted with H₂O (10 mL), and extracted with EtOAc (3×20 mL). The combined organics were dried (Na₂SO₄) and filtered. Purification by chromatography (silica gel, 0-40% EtOAc/hexanes) afforded 90 mg (72%) of the title compound. MS (ESI): mass calcd. for C₁₂H₅ClF₂N₂, 250.0; m/z found, 250.9 [M+H]⁺. ¹H NMR (500 MHz, DMSO-d₆) δ 8.72 (t, J=1.38 Hz, 1H), 8.28 (dd, J=5.07, 0.69 Hz, 1H), 7.90 (dd, J=6.50, 1.75 Hz, 2H), 7.72 (d, J=5.13 Hz, 1H).

### Intermediate 135: 2-((3-Chloropyridin-4-yl)ethynyl)-5-fluoropyridine

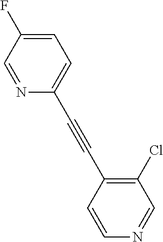 [SMILES: Fc1ccc(C#Cc2ccncc2Cl)nc1]

The title compound was prepared in a manner analogous to Intermediate 134, except using 4-bromo-3-chloropyridine instead of 4-bromo-3-chloro-2-fluoropyridine. MS (ESI): mass calcd. for C₁₂H₆ClFN₂, 232.0; m/z found, 233.1 [M+H]⁺. ¹H NMR (400 MHz, CDCl₃): δ 8.69 (s, 1H), 8.55 (d, J=2.88 Hz, 1H), 8.51 (d, J=5.00 Hz, 1H), 7.65 (dd, J=8.63, 4.50 Hz, 1H), 7.51-7.42 (m, 2H).

### Intermediate 136: 7-((5-Fluoropyridin-2-yl)ethynyl)-1-methyl-1H-pyrazolo[4,3-b]pyridine

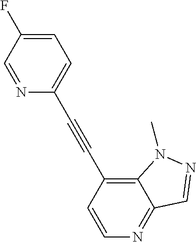 [SMILES: Cn1ncc2nccc(C#Cc3ccc(F)cn3)c21]

To a pressure vial was added 7-chloro-1-methyl-1H-pyrazolo[4,3-b]pyridine (100 mg, 0.60 mmol), 2-ethynyl-5-fluoropyridine (108 mg, 0.90 mmol), XPhos Pd G3 (25 mg, 0.03 mmol), Cs₂CO₃ (583 mg, 1.8 mmol), and acetonitrile (1.2 mL). The resulting mixture was degassed with N₂ and heated for 2 h at 80° C. The reaction was cooled to room temperature and partitioned between ethyl acetate and H₂O. The layers were separated and the aqueous was extracted with ethyl acetate (2×5 mL). The organic layers were combined and washed with brine (5 mL), dried (Na₂SO₄), filtered through Celite®, and condensed. Purification by chromatography (silica gel, 0-100% EtOAc/hexanes) afforded 72 mg (48%) of the title compound. MS (ESI): mass calcd. for C₁₄H₉FN₄, 232.0; m/z found, 233.1 [M+H]⁺. ¹H NMR (400 MHz, DMSO-d₆) δ 8.72 (dt, J=2.69, 0.72 Hz, 1H), 8.57 (d, J=4.50 Hz, 1H), 8.39 (s, 1H), 7.88-7.96 (m, 2H), 7.62 (d, J=4.63 Hz, 1H), 4.41 (s, 3H).

### Intermediate 137: 4-((5-Fluoropyridin-2-yl)ethynyl)-1-((2-(trimethylsilyl)ethoxy)methyl)-1H-pyrrolo[2,3-b]pyridine-3-carbonitrile

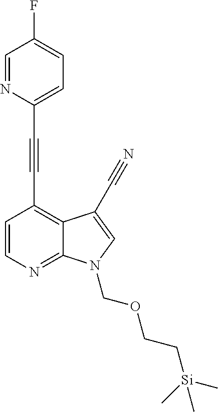 [SMILES: C[Si](C)(C)CCOCn1cc(C#N)c2c(C#Cc3ccc(F)cn3)ccnc21]

Step A. 4-Chloro-1-((2-(trimethylsilyl)ethoxy)methyl)-1H-pyrrolo[2,3-b]pyridine-3-carbonitrile. (2-(Chloromethoxy)ethyl)trimethylsilane (4.49 mL, 25.4 mmol) was added dropwise to a 0° C. (ice/water) solution consisting of 4-chloro-1H-pyrrolo[2,3-b]pyridine-3-carbonitrile (3.00 g, 16.9 mmol), Et₃N (7.04 mL, 50.6 mmol), and dichloromethane (30 mL). The resultant mixture was stirred for 12 hours. The reaction mixture was gradually warmed to room-temperature then diluted with H₂O (50 mL) and extracted with dichloromethane (150 mL×3). The combined organic extracts were dried over anhydrous Na₂SO₄, filtered, and concentrated to dryness under reduced pressure. The resulting residue was purified by FCC (SiO₂, eluent: petroleum ether:ethyl acetate=1:0 to 5:1) to afford the title compound (1.3 g, 24% yield) as a white solid. LC-MS (ESI): mass calcd. for C₁₄H₁₈ClN₃OSi 307.09 m/z found 308.2 [M+H]⁺.

Step B. 4-((5-Fluoropyridin-2-yl)ethynyl)-1-((2-(trimethylsilyl)ethoxy)methyl)-1H-pyrrolo[2,3-b]pyridine-3-carbonitrile. 4-Chloro-1-((2-(trimethylsilyl)ethoxy)methyl)-1H-pyrrolo[2,3-b]pyridine-3-carbonitrile (500 mg, 1.62 mmol), 2-ethynyl-5-fluoropyridine (590 mg, 4.87 mmol), CuI (130 mg, 0.683 mmol), Et₃N (15 mL), and DMF (15 mL) were added to a 100 mL round-bottomed flask. The resultant mixture was sparged with N₂ for 5 minutes and then treated with PdCl₂(Cy*Phine)₂ (230 mg, 0.179 mmol). The resultant mixture was sparged with N₂ for another 5 minutes and heated at 125° C. for 8 hours. The reaction mixture was gradually cooled to room-temperature, then diluted with H₂O (50 mL), and extracted with ethyl acetate (250 mL×2). The combined organic extracts were dried over anhydrous Na₂SO₄, filtered, and concentrated to dryness under reduced pressure. The resulting residue was purified by FCC (SiO₂, eluent: petroleum ether:ethyl acetate=1:0 to 4:1) to afford the product (400 mg, 60%) as a brown solid. MS (ESI): mass calcd. for C₂₁H₂₁FN₄OSi, 392.1; found 393.1 [M+H]⁺.

### Intermediate 138: (R)-5-Fluoro-5,6-dihydro-4H-pyrrolo[1,2-c][1,2,3]oxadiazol-7-ium-3-olate

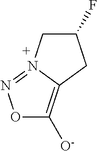

Step A: (2S,4R)-4-Fluoropyrrolidine-2-carboxylic acid hydrochloride. To a suspension of (2S,4R)-1-tert-butoxycarbonyl-4-fluoro-pyrrolidine-2-carboxylic acid (3 g, 12.9 mmol) in dichloromethane (60 mL) was added hydrogen chloride (4.2 M in 1,4-dioxane, 30 mL, 126 mmol). The reaction mixture was stirred at room temperature for 2 h. The reaction mixture was evaporated. To the residue was added diethyl ether (20 mL) and the mixture was evaporated. This procedure was repeated twice to remove excess hydrochloric acid to give the title compound (2.15 g, 12.678 mmol, 99%) as a white powder. MS (ESI): mass calcd. for C₅H₈FNO₂·HCl, 133.1; found 134.2 [M+H]⁺.

Step B: (R)-5-Fluoro-5,6-dihydro-4H-pyrrolo[1,2-c][1,2,3]oxadiazol-7-ium-3-olate. The title compound was prepared in a manner analogous to Intermediate 3, Step A-B except using (2S,4R)-4-fluoropyrrolidine-2-carboxylic acid hydrochloride instead of 4-nitrosomorpholine-3-carboxylic acid and diethyl ether instead of acetonitrile. MS (ESI): mass calcd. for C₅H₅FN₂O₂, 144.0; found 145.1 [M+H]⁺.

### Intermediate 139: tert-Butyl 4-(4,4,5,5-tetramethyl-1,3,2-dioxaborolan-2-yl)-1H-pyrrolo[2,3-b]pyridine-1-carboxylate

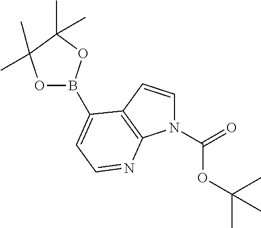 [SMILES: CC(C)(C)OC(=O)n1ccc2c(B3OC(C)(C)C(C)(C)O3)ccnc21]

Step A: tert-Butyl 4-bromo-1H-pyrrolo[2,3-b]pyridine-1-carboxylate. To a suspension of 4-bromo-7-azaindole (4.3 g, 21.8 mmol) in DCM (85 mL) was added TEA (4.55 mL, 32.6 mmol) and 4-dimethylamino-pyridine (267 mg, 2.18 mmol). The reaction was cooled to 0° C. and di-tert-butyl dicarbonate (6 mL, 26.1 mmol) was added dropwise. The reaction mixture was allowed to warm to room temperature and stir for 1 h then the reaction was diluted with DCM (400 mL). The organic layer was washed with H₂O (2×250 mL). The aqueous phase was extracted with dichloromethane (200 mL). The organic layers were combined and dried over MgSO₄, filtered and evaporated. Purification by chromatography (silica gel, 20:1 n-heptane:EtOAc) afforded 5.54 g (85%) of the title compound. MS (ESI): mass calcd. for C₁₂H₁₃BrN₂O₂, 296.0; m/z found, 243.0 [M+H-tBu]⁺.

Step B: tert-Butyl 4-(4,4,5,5-tetramethyl-1,3,2-dioxaborolan-2-yl)-1H-pyrrolo[2,3-b]pyridine-1-carboxylate. The title compound was prepared in a manner analogous to Intermediate 21, Step B except using tert-butyl 4-bromo-1H-pyrrolo[2,3-b]pyridine-1-carboxylate instead of 4-bromo-1-((2-(trimethylsilyl)ethoxy)methyl)-1H-pyrrolo[2,3-b]pyridine. ¹H NMR (500 MHz, DMSO-d₆) δ 8.39 (d, J=4.6 Hz, 1H), 7.82 (d, J=3.9 Hz, 1H), 7.45 (d, J=4.6 Hz, 1H), 6.84 (d, J=3.9 Hz, 1H), 1.34 (s, 12H), 1.05 (s, 9H).

### Intermediate 140: 2-(4-Fluorophenyl)-3-iodo-4,5,6,7-tetrahydropyrazolo[1,5-a]pyridine

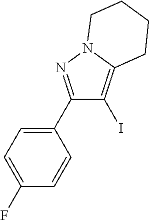 [SMILES: Fc1ccc(-c2nn3c(c2I)CCCC3)cc1]

The title compound was made in a manner analogous to Intermediate 37, Steps A-B, except using 4,5,6,7-tetrahydro-[1,2,3]oxadiazolo[3,4-a]pyridin-8-ium-3-olate instead of 6,7-dihydro-4H-[1,2,3]oxadiazolo[4,3-c][1,4]oxazin-8-ium-3-olate (Intermediate 2), 4-fluorophenylacetylene instead of 2-ethynyl-5-fluoropyridine and mesitylene instead of xylenes in Step A; and N-iodosuccinimide instead of N-bromosuccinimide and acetonitrile instead of DMF in Step B. MS (ESI): mass calcd. for C₁₃H₁₂FIN₂, 342.0; found 343.0 [M+H]⁺. ¹H NMR (300 MHz, Acetonitrile-d₃) δ 7.90-7.79 (m, 2H), 7.26-7.13 (m, 2H), 4.19-4.07 (m, 2H), 2.75-2.65 (m, 2H), 2.10-2.00 (m, 2H), 1.96-1.85 (m, 2H).

### Intermediate 141: 4,4-Difluoro-5,6-dihydro-4H-pyrrolo[1,2-c][1,2,3]oxadiazol-7-ium-3-olate

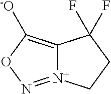

Step A: 3,3-Difluoropyrrolidine-2-carboxylic acid. To a solution consisting of 1-(tert-butoxycarbonyl)-3,3-difluoropyrrolidine-2-carboxylic acid (1.8 g, 7.2 mmol) and 1,4-dioxane (5 mL) at room-temperature was added HCl/1,4-dioxane (5 mL) and the mixture was stirred for 2 hours at room-temperature. The mixture was concentrated to dryness under reduced pressure to give the title product (1.8 g) as a brown oil which was used in the next step without further purification. ¹H NMR (400 MHz, DMSO-d₆) δ 13.90-12.76 (m, 1H), 4.41-4.29 (m, 1H), 3.57 (s, 2H), 1.43-1.33 (m, 1H), 1.43-1.33 (m, 1H).

Step B: 4,4-Difluoro-5,6-dihydro-4H-pyrrolo[1,2-c][1,2,3]oxadiazol-7-ium-3-olate. The title compound was prepared in a manner analogous to Intermediate 2, Steps A-B, except using 3,3-difluoropyrrolidine-2-carboxylic acid instead of morpholine-3-carboxylic acid in Step A. MS (ESI): mass calcd. for C₅H₄F₂N₂O₂ 162.1; m/z found 162.7 [M+H]⁺.

### Intermediate 142: 3-Bromo-4,4-difluoro-2-(5-fluoropyridin-2-yl)-5,6-dihydro-4H-pyrrolo[1,2-b]pyrazole

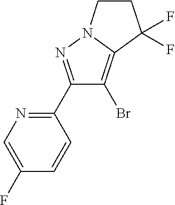 [SMILES: Fc1ccc(-c2nn3c(c2Br)C(F)(F)CC3)nc1]

The title compound was prepared in a manner analogous to Intermediate 37, Steps A-B, except using 4,4-difluoro-5,6-dihydro-4H-pyrrolo[1,2-c][1,2,3]oxadiazol-7-ium-3-olate (Intermediate 141) instead of 6,7-dihydro-4H-[1,2,3]oxadiazolo[4,3-c][1,4]oxazin-8-ium-3-olate (Intermediate 2) in Step A. MS (ESI): mass calcd. for C₁₁H₇BrF₃N₃ 317.0; m/z found 317.8 [M+H]⁺.

### Intermediate 143: 3-Bromo-2-(4-fluorophenyl)-4,5,6,7-tetrahydro-4,7-methanopyrazolo[1,5-a]pyridine

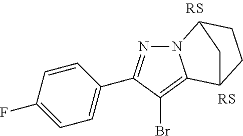 [SMILES: Fc1ccc(-c2nn3c(c2Br)C2CCC3C2)cc1]

Step A: Benzyl 2-(3-(methoxycarbonyl)cyclopentyl)hydrazine-1-carboxylate. A mixture of methyl 3-oxocyclopentanecarboxylate (14.2 g, 100 mmol), benzyl hydrazinecarboxylate (16.6 g, 100 mmL), MeOH (200 mL), AcOH (100 mL) and NaBH₃CN (18.86 g, 300 mmol) were stirred at room-temperature for 16 hours. The reaction was concentrated to dryness and quenched with sat·NaHCO₃ solution (150 mL) and water (100 mL), extracted with EtOAc (150 mL×2). The combined organic extracts were washed with brine (50 mL), dried over anhydrous Na₂SO₄, filtered, and concentrated to dryness under reduced pressure to give the crude oil, which was purified with FCC (SiO₂, eluent: petroleum ether:ethyl acetate=3:1, R^{f}=0.5) to afford the title compound as colorless oil (20 g, 68%). MS (ESI): mass calcd. for C₁₅H₂₀N₂O₄ 292.3; m/z found 293.0 [M+H]⁺.

Step B: 3-(2-((Benzyloxy)carbonyl)hydrazineyl)cyclopentane-1-carboxylic acid. A mixture of benzyl 2-(3-(methoxycarbonyl)cyclopentyl)hydrazinecarboxylate (2.9 g, 9.9 mmol) and EtOH (30 mL) was added NaOH (30 mL, 37.5 mmol, 1.25 M in H₂O). The mixture was stirred at room-temperature for 2 hours. The mixture was concentrated to remove most of EtOH, diluted with H₂O, acidified with 2 M HCl to pH=4-6, extracted with EtOAc (40 mL×3). The combined organic extracts were washed with brine (30 mL), dried over anhydrous Na₂SO₄, filtered, and concentrated to dryness under reduced pressure to afford product (1.7 g, 81% yield).

Step C: Benzyl (3-oxo-2-azabicyclo[2.2.1]heptan-2-yl)carbamate. To a mixture of 3-(2-((benzyloxy)carbonyl)hydrazineyl)cyclopentane-1-carboxylic acid (2.67 g, 9.6 mmol), TEA (4.85 g, 48 mmol) and DCM (80 mL) was added T3P® (7.3 mL). The mixture was stirred at room-temperature for 16 hours. The reaction was concentrated to dryness and quenched with sat·NaHCO₃ solution (50 mL) and water (50 mL), extracted with EtOAc (60 mL×2). The combined organic extracts were washed with brine (20 mL), dried over anhydrous Na₂SO₄, filtered, and concentrated to dryness to give crude oil, which was purified with by FCC (SiO₂, eluent: petroleum ether:ethyl acetate=1:1, R^{f}=0.2) to afford the title compound (1.2 g, 48%) as colorless oil. MS (ESI): mass calcd. for C₁₄H₁₆N₂O₃ 260.3; m/z found 261.2 [M+H]⁺.

Step D: 2-Amino-2-azabicyclo[2.2.1]heptan-3-one tosylate salt. Benzyl (3-oxo-2-azabicyclo[2.2.1]heptan-2-yl)carbamate (2.2 g, 8.45 mmol), TsOH (1.45 g, 8.45 mmol), MeOH (50 mL) and wet Pd/C (100 mg) was added to a round-bottomed flask. The resulting mixture was stirred under H₂ (balloon) at room-temperature for 24 hours. The suspension was filtered through a pad of Celite® and the pad washed with MeOH. The filtrate was concentrated to dryness under reduced pressure to afford the title product (2.2 g, 87%) as a white solid which was used directly in next step.

Step E: Ethyl (E)-3-(4-fluorophenyl)-3-((3-oxo-2-azabicyclo[2.2.1]heptan-2-yl)imino)propanoate. A mixture of 2-amino-2-azabicyclo[2.2.1]heptan-3-one tosylate salt (2.2 g, 7.37 mmol), ethyl 3-(4-fluorophenyl)-3-oxopropanoate (1.2 g, 5.67 mmol), 4 Å MS sieves (2 g) and pyridine (15 mL) was stirred at room-temperature for 24 hours. The reaction mixture was concentrated to dryness and quenched with NaHCO₃ solution (50 mL), extracted with EtOAc (50 mL). The combined organic extracts were washed with brine (10 mL), dried over anhydrous Na₂SO₄, filtered, and concentrated to dryness under reduced pressure. The resulting residue was purified by FCC (SiO₂, eluent: petroleum ether:ethyl acetate=1:1) to afford the title compound (1.0 g, 55%) as a yellow oil. MS (ESI): mass calcd. for C₁₇H₁₉FN₂O₃ 318.3; m/z found 319.2 [M+H]⁺.

Step F: Ethyl 2-(4-fluorophenyl)-4,5,6,7-tetrahydro-4,7-methanopyrazolo[1,5-a]pyridine-3-carboxylate. A mixture of ethyl 3-(4-fluorophenyl)-3-oxopropanoate (1.0 g, 3.14 momL), Cs₂CO₃ (2.57 g, 7.85 mmol) and DMF (15 mL) was stirred at 90° C. for 2 hours. The reaction was concentrated to dryness and quenched with NH₄Cl solution (50 mL), extracted with EtOAc (20 mL×2). The combined organic extracts were washed with brine (10 mL), dried over anhydrous Na₂SO₄, filtered, and concentrated to dryness under reduced pressure to give the title product (800 mg, 85% yield). MS (ESI): mass calcd. for C₁₇H₁₇FN₂O₂ 300.3; m/z found 301.2 [M+H]⁺.

Step G: 2-(4-Fluorophenyl)-4,5,6,7-tetrahydro-4,7-methanopyrazolo[1,5-a]pyridine-3-carboxylic acid. Ethyl 2-(4-fluorophenyl)-4,5,6,7-tetrahydro-4,7-methanopyrazolo[1,5-a]pyridine-3-carboxylate (600 mg, 12.1 mmol) was added to a solution of LiOH·H₂O (439.7 mg, 10.48 mmol), MeOH (10 mL) and H₂O (2 mL). The reaction was stirred at 100° C. for 2 hours. The reaction mixture was concentrated to a small volume and diluted with H₂O, acidified with HCl (4 M) to pH=4-6. The mixture was extracted with ethyl acetate (20 mL×3). The combined organic extracts were dried over anhydrous Na₂SO₄, filtered, and concentrated to dryness under reduced pressure to afford the title compound (550 mg, 93%) as a yellow oil. MS (ESI): mass calcd. for C₁₅H₁₃FN₂O₂ 272.3; m/z found 273.1 [M+H]⁺.

Step H: 3-Bromo-2-(4-fluorophenyl)-4,5,6,7-tetrahydro-4,7-methanopyrazolo[1,5-a]pyridine. NBS (637.35 mg, 3.58 mmol) was added to a solution containing of (2-(4-fluorophenyl)-4,5,6,7-tetrahydro-4,7-methanopyrazolo[1,5-a]pyridine-3-carboxylic acid (650 mg, 2.387 mmol) and DMF (15 mL). The resulted solution was heated at 50° C. for 5 hours. The reaction mixture was diluted with H₂O (50 mL), extracted with EtOAc (20 mL×2). The combined organic extracts were washed with brine (10 mL), dried over anhydrous Na₂SO₄, filtered, and concentrated to dryness under reduced pressure. The resulting residue was purified by FCC (SiO₂, eluent: petroleum ether:ethyl acetate=1:1) to afford the title compound (720 mg, 98%) as a yellow solid. MS (ESI): mass calcd. for C₁₄H₁₂BrFN₂ 306.2; m/z found 307.1 [M+H]⁺.

### Intermediate 144: 3-Bromo-2-(5-fluoropyridin-2-yl)-6,6-dimethyl-6,7-dihydro-4H-pyrazolo[5,1-c][1,4]oxazine-7,7-d₂

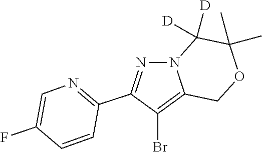 [SMILES: [2H]C1([2H])n2nc(-c3ccc(F)cn3)c(Br)c2COC1(C)C]

Step A: 2-Methylpropane-1,1-d₂-1,2-diol. LiAlH₄ (4.20 g, 100 mmol) was added to a 0° C. (ice/water) solution consisting of methyl 2-hydroxy-2-methylpropanoate (4.7 g, 40 mmol) and THF (100 mL) under N₂. The resulting mixture was stirred at room temperature for 16 hours under N₂. To the mixture 20 mL THF was added followed by slow addition of H₂O (4.2 mL), then 15% NaOH (aq, 4.2 mL) was added. The resulting mixture was stirred at room temperature for 0.5 hours. Then H₂O (12.6 mL) was added. The mixture was filtered and the filter cake was washed with THF (20 mL×3). The filtrate was under reduced pressure to afford the title compound (3.0 g, 82%). ¹H NMR (400 MHz, CDCl₃) δ 2.25-2.10 (m, 1H), 2.04 (br s, 1H), 1.75 (br s, 1H), 1.20 (s, 6H).

Step B: 2-Hydroxy-2-methylpropyl-1,1-d₂ 4-methylbenzenesulfonate. Toluene sulfonyl chloride (6.21 g, 32.6 mmol) was added to a mixture of 2-methylpropane-1,2-diol (3.0 g, 33 mmol), triethylamine (9.1 ml, 65 mmol), 4-dimethyl aminopyridine (399 mg, 3.27 mmol) and DCM (120 mL). The reaction was stirred at room temperature for 16 hours. The reaction solution was concentrated under reduced pressure and purified by FCC (SiO₂, ethyl acetate:petroleum ether=1:10 to 1:3) to afford the title compound as a colorless oil (3.2 g, 40%). ¹H NMR (400 MHz, CDCl₃) δ 7.79 (d, J=8.2 Hz, 2H), 7.35 (br d, J=8.2 Hz, 2H), 2.44 (s, 3H), 1.25-1.15 (m, 6H).

Step C: 1-(3-(5-Fluoropyridin-2-yl)-5-(hydroxymethyl)-1H-pyrazol-1-yl)-2-methylpropan-1,1-d₂-2-ol. 2-Hydroxy-2-methylpropyl-1,1-d₂ 4-methylbenzenesulfonate (250 mg, 1.02 mmol), 2-(5-(((tert-butyldimethylsilyl)oxy)methyl)-1H-pyrazol-3-yl)-5-fluoropyridine (Intermediate 35, 312 mg, 1.02 mmol), Cs₂CO₃ (1.65 g, 5.06 mmol), KI (168 mg, 1.01 mmol), and DMA (8 mL) were added to a 20 mL microwave tube. The resulting mixture was heated at 120° C. via microwave irradiation for 0.5 hours. The reaction mixture was cooled to room-temperature. The mixture was poured into water (30 mL) and extracted with ethyl acetate (30 mL×3). The combined organic layers were washed with brine (30 mL), dried over anhydrous Na₂SO₄, filtered and concentrated under reduced pressure. The resulting residue was purified by FCC (SiO₂, petroleum ether:ethyl acetate) to afford the title compound (300 mg, 74.42% purity) as a clean oil. MS (ESI): mass calcd. for C₁₃H₁₄D₂FN₃O₂ 267.3; m/z found 268.1 [M+H]⁺.

Step D: 2-(5-fluoropyridin-2-yl)-6,6-dimethyl-6,7-dihydro-4H-pyrazolo[5,1-c][1,4]oxazine-7,7-d₂. H₃PO₄ (1 mL) was added to a solution consisting of 1-(3-(5-fluoropyridin-2-yl)-5-(hydroxymethyl)-1H-pyrazol-1-yl)-2-methylpropan-1,1-d₂-2-ol (300 mg, 1.12 mmol), and toluene (10 mL). The mixture was stirred at 110° C. for 16 hours. The mixture was added into H₂O (20 mL) and adjust to pH=8 with aq·NaOH (2 M). Then mixture was extracted with ethyl acetate (30 mL×3) and the combined organic extracts were washed with brine (10 mL), dried over anhydrous Na₂SO₄, filtered, and concentrated to dryness under reduced pressure. The resulting residue was purified by FCC (SiO₂, petroleum ether:ethyl acetate=10:1 to 2:1) to afford the title compound (180 mg, 57%) as a white solid. MS (ESI): mass calcd. for C₁₃H₁₂D₂FN₃O 249.3; m/z found 250.2 [M+H]⁺. ¹H NMR (400 MHz, DMSO-d₆) δ 8.52 (d, J=2.9 Hz, 1H), 7.94-7.88 (m, 1H), 7.76-7.67 (m, 1H), 6.56 (s, 1H), 4.80 (s, 2H), 1.26 (s, 6H).

Step E: 3-Bromo-2-(5-fluoropyridin-2-yl)-6,6-dimethyl-6,7-dihydro-4H-pyrazolo[5,1-c][1,4]oxazine-7,7-d₂. NBS (154 mg, 0.865 mmol) was added to a solution consisting of 2-(5-fluoropyridin-2-yl)-6,6-dimethyl-6,7-dihydro-4H-pyrazolo[5,1-c][1,4]oxazine-7,7-d₂ (180 mg, 0.722 mmol) and DCM (5 mL). The resulting mixture was stirred at room-temperature for 2 hours. The reaction mixture was quenched with water (20 mL) and extracted with DCM (10 mL×3). The combined organic extracts were dried over Na₂SO₄, filtered, and concentrated to dryness under reduced pressure. The resulting residue was purified by FCC (SiO₂, petroleum ether:ethyl acetate=20:1 to 3:1) to afford the title compound (100 mg, 36%) as a white solid. MS (ESI): mass calcd. for C₁₃H₁₁BrD₂FN₃O 327.0; m/z found 328.0 [M+H]⁺.

### Intermediate 145: 3-Bromo-2-(4-fluorophenyl)-4,5,6,7-tetrahydro-4,7-ethanopyrazolo[1,5-a]pyridine

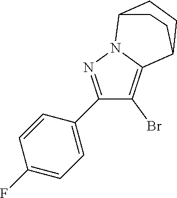 [SMILES: Fc1ccc(-c2nn3c(c2Br)C2CCC3CC2)cc1]

The title compound was prepared in a manner analogous to Intermediate 143, Steps A-E, except using methyl 4-oxocyclohexanecarboxylate instead of methyl 3-oxocyclopentanecarboxylate in Step A. MS (ESI): mass calcd. for C₁₅H₁₄BrFN₂ 320.0; m/z found 321.0 [M+H]⁺.

### Intermediate 146: 3-Bromo-2-(5-fluoropyridin-2-yl)-4,5,6,7-tetrahydro-4,7-methanopyrazolo[1,5-a]pyridine

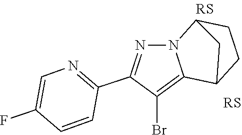 [SMILES: Fc1ccc(-c2nn3c(c2Br)C2CCC3C2)nc1]

The title compound was prepared in a manner analogous to Intermediate 143, Steps A-H, except using methyl 3-(5-fluoropyridin-2-yl)-3-oxopropanoate instead of ethyl 3-(4-fluorophenyl)-3-oxopropanoate in Step E. MS (ESI): mass calcd. for C₁₃H₁₁BrFN₃ 307.0; m/z found 308.1 [M+H]⁺.

### Intermediate 147: 2-(3-Bromo-4,5,6,7-tetrahydropyrazolo[1,5-a]pyridin-2-yl)thiazole

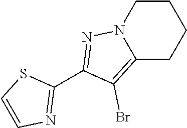 [SMILES: Brc1c(-c2nccs2)nn2c1CCCC2]

The title compound was prepared in a manner analogous to Intermediate 37, Steps A-B, except using 4,5,6,7-tetrahydro-[1,2,3]oxadiazolo[3,4-a]pyridin-8-ium-3-olate (Intermediate 9) and 2-ethynylthiazole in Step A. MS (ESI): mass calcd. For C₁₀H₁₀BrN₃S, 282.97 m/z found, 283.9 [M+H]⁺. ¹H NMR (400 MHz, DMSO-d₆): 7.92 (d, J=3.3 Hz, 1H), 7.74 (d, J=3.3 Hz, 1H), 4.12 (t, J=6.0 Hz, 2H), 2.68 (t, J=6.4 Hz, 2H), 2.03-1.94 (m, 2H), 1.89-1.79 (m, 2H).

### Intermediate 148: 4-Bromo-3-fluoro-1H-pyrazolo[3,4-b]pyridine

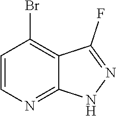 [SMILES: Fc1n[nH]c2nccc(Br)c12]

To a solution of 4-bromo-1H-pyrazolo[3,4-b]pyridine (1.36 g, 6.89 mmol) in CCl₄ (10 mL) was added xenon difluoride (3.5 g, 20.7 mmol). The reaction mixture was purged with N₂, the vial sealed, and the resulting mixture was stirred at 40° C. After three hours the internal pressure was released. After 19 hours the reaction mixture was quenched with sat. bicarb. (30 mL). The resulting mixture was extracted with DCM (3×30 mL). The combined organic layers were dried over Na₂SO₄, filtered, and concentrated under reduced pressure. The resulting residue was purified by FCC (SiO₂, EtOAc: DCM=0-50%), then further purified by acidic ACCQ-prep. HPLC (0.05% TFA in H₂O and 0.05% TFA in CH₃CN) to afford the title compound as a brownish solid (0.16 g, 11%). MS (ESI): mass calcd. for C₆H₃BrFN₃ 214.9; m/z found 216.1 [M+H]⁺. ¹H NMR (400 MHz, CD₃OD): δ 8.37 (d, J=5.0 Hz, 1H), 7.49 (d, J=5.0 Hz, 1H).

### Intermediate 150: (R)-3-Bromo-5-fluoro-2-(4-fluorophenyl)-5,6-dihydro-4H-pyrrolo[1,2-b]pyrazole

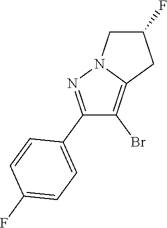 [SMILES: Fc1ccc(-c2nn3c(c2Br)C[C@@H](F)C3)cc1]

Step A: (R)-5-Fluoro-5,6-dihydro-4H-pyrrolo[1,2-c][1,2,3]oxadiazol-7-ium-3-olate (Intermediate 138). The title compound was prepared in a manner analogous to 5,6-dihydro-4H-pyrrolo[1,2-c][1,2,3]oxadiazol-7-ium-3-olate (Intermediate 1), except using (2S,4R)-4-fluoropyrrolidine-2-carboxylic acid instead of proline. ¹H NMR (400 MHz, DMSO-d₆) δ 6.04-5.80 (m, 1H), 5.00-4.79 (m, 2H), 3.33-3.17 (m, 1H), 3.08-2.93 (m, 1H).

Step B: (R)-5-Fluoro-2-(4-fluorophenyl)-5,6-dihydro-4H-pyrrolo[1,2-b]pyrazole. A solution of 4-fluorophenylacetylene (2.4 mL, 20.9 mmol), (R)-5-fluoro-5,6-dihydro-4H-pyrrolo[1,2-c][1,2,3]oxadiazol-7-ium-3-olate (Intermediate 138, 1 g, 6.93 mmol), in xylenes (10 mL), was sparged with N₂ and heated employing microwave irradiation at 200° C. for 1 hour. The reaction mixture was cooled. Purification (FCC, SiO₂, eluent: petroleum ether:ethyl acetate=1:0 to 2:1) afforded the title compound (1.1 g, 71% yield) as a yellow solid. ¹H NMR (400 MHz, DMSO-d₆): 7.86-7.75 (m, 2H), 7.28-7.15 (m, 2H), 6.54 (s, 1H), 6.00-5.72 (m, 1H), 4.55-4.24 (m, 2H), 3.32-3.25 (m, 1H), 3.18-2.99 (m, 1H).

Step C: (R)-3-Bromo-5-fluoro-2-(4-fluorophenyl)-5,6-dihydro-4H-pyrrolo[1,2-b]pyrazole. A solution of (R)-5-fluoro-2-(4-fluorophenyl)-5,6-dihydro-4H-pyrrolo[1,2-b]pyrazole (1 g, 4.5 mmol), NBS (974 mg, 5.47 mmol), and dichloromethane (20 mL) was stirred at room-temperature for 2 hours. The reaction mixture was poured into water (20 mL) and extracted with DCM (20 mL×3). The combined organic layers were dried over anhydrous Na₂SO₄, filtered, and concentrated to dryness under reduced pressure. Purification of the resulting residue (FCC SiO₂, eluent: petroleum ether:ethyl acetate=1:0 to 2:1) afforded the title compound (1.2 g, 88% yield) as a yellow solid. MS (ESI): mass calcd. for C₁₂H₉BrF₂N₂ 298.0; m/z found 299.0 [M+H]⁺. ¹H NMR (400 MHz, DMSO-d₆): 7.88-7.78 (m, 2H), 7.38-7.25 (m, 2H), 6.00-5.78 (m, 1H), 4.64-4.34 (m, 2H), 3.36-3.28 (m, 1H), 3.15-2.98 (m, 1H).

### Intermediate 151: 3-Bromo-2-(4-fluorophenyl)-5,5-dimethyl-5,6-dihydro-4H-pyrrolo[1,2-b]pyrazole

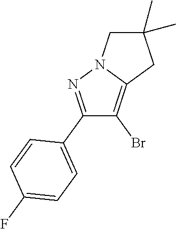 [SMILES: CC1(C)Cc2c(Br)c(-c3ccc(F)cc3)nn2C1]

The title compound was prepared in a manner analogous to (R)-3-bromo-5-fluoro-2-(4-fluorophenyl)-5,6-dihydro-4H-pyrrolo[1,2-b]pyrazole (Intermediate 150) Steps A-C; using (R)-4,4-dimethylpyrrolidine-2-carboxylic acid instead of (2S,4R)-4-fluoropyrrolidine-2-carboxylic acid in Step A; and using diphenyl ether instead of xylenes in Step B. MS (ESI): mass calcd. for C₁₄H₁₄BrFN₂ 308.0; m/z found 309.0 [M+H]⁺.

### Intermediate 152: 3-Bromo-2-(5-fluoropyridin-2-yl)-5,5-dimethyl-5,6-dihydro-4H-pyrrolo[1,2-b]pyrazole

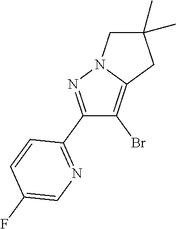 [SMILES: CC1(C)Cc2c(Br)c(-c3ccc(F)cn3)nn2C1]

The title compound was prepared in a manner analogous to (R)-3-bromo-5-fluoro-2-(4-fluorophenyl)-5,6-dihydro-4H-pyrrolo[1,2-b]pyrazole (Intermediate 150) Steps A-C; using (R)-4,4-dimethylpyrrolidine-2-carboxylic acid instead of (2S,4R)-4-fluoropyrrolidine-2-carboxylic acid in Step A; using 2-ethynyl-5-fluoropyridine instead of 4-fluorophenylacetylene, using diphenyl ether instead of xylenes, and heating the reaction mixture to 240° C. for 1.5 hours in Step B. MS (ESI): mass calcd. for C₁₃H₁₃BrFN₃ 309.0; m/z found 310.0 [M+H]⁺.

### Intermediate 153: 3-Bromo-2-(4-fluorophenyl)-5,5-bis(methyl-d₃)-5,6-dihydro-4H-pyrrolo[1,2-b]pyrazole

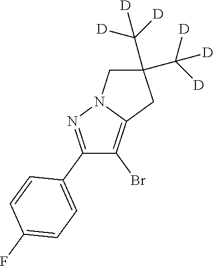 [SMILES: [2H]C([2H])([2H])C1(C([2H])([2H])[2H])Cc2c(Br)c(-c3ccc(F)cc3)nn2C1]

Step A. Di-tert-butyl 4,4-bis(methyl-d₃)-5-oxopyrrolidine-1,2-dicarboxylate. To a cooled solution (−70° C.; dry ice/acetone) of (S)-di-tert-butyl 5-oxopyrrolidine-1,2-dicarboxylate (10.0 g, 35.0 mmol) in anhydrous THF (150 mL) was added LiHMDS (77 mL, 1 M in THF, 77 mmol) dropwise. The resultant mixture was stirred at −70° C. for 1 hour. CD₃I (4.8 mL, 77 mmol) in THF (100 mL) was added dropwise to above solution. The resultant mixture was stirred for 12 hours. The reaction mixture was gradually warmed to room-temperature. The reaction mixture was poured into sat. NH₄Cl (50 mL), and extracted with ethyl acetate (200 mL×3). The combined organic extracts were dried over anhydrous Na₂SO₄, filtered, and concentrated to dryness under reduced pressure. Purification (FCC, SiO₂, eluent: petroleum ether:ethyl acetate=10:1 to 9:1) afforded the title compound (6.9 g, 55% yield, 90% purity) as a yellow solid.

Step B. Di-tert-butyl 4,4-bis(methyl-d₃)pyrrolidine-1,2-dicarboxylate. BH₃·THF (78 mL, 1 M in THF, 78 mmol) was added dropwise to a 0° C. (ice/water) solution of (S)-di-tert-butyl 5-oxo-4,4-bis(tri-D-methyl)pyrrolidine-1,2-dicarboxylate (5.0 g, 16 mmol) in THF (100 mL). The resultant solution was stirred at room-temperature for 16 hours. The reaction mixture was quenched with ice-water (150 mL) at 0° C. and stirred at 0° C. for 0.5 hours. The resultant solution was extracted with ethyl acetate (150 mL×3). The combined organic extracts were washed with brine (150 mL), dried over anhydrous Na₂SO₄, filtered, and concentrated to dryness under reduced pressure. Purification (preparative HPLC using a Phenomenex Genimi NX C18 150 mm×40 mm×5 m column (eluent: 45% to 75% (v/v) CH₃CN and H₂O with 0.225% FA)) afforded the title compound (1.6 g, 33%) as white solid. MS (ESI): mass calcd. for C₁₆H₂₃D₆NO₄ 305.3; m/z; found 193.9 [M−2tBu+H]⁺.

Step C. 4,4-Bis(methyl-d₃)pyrrolidine-2-carboxylic acid hydrochloride. To a solution consisting of (S)-di-tert-butyl 4,4-bis(tri-D-methyl)pyrrolidine-1,2-dicarboxylate (1.6 g, 5.2 mmol) and 1,4-dioxane (10 mL) was added HCl/1,4-dioxane (10 mL, 40 mmol) dropwise. The reaction mixture was stirred at 50° C. for 16 hours. The reaction mixture was concentrated to dryness under reduced pressure to afford the title compound (1.0 g) as a white solid, which was used without further purification.

Step D: 3-Bromo-2-(4-fluorophenyl)-5,5-bis(methyl-d₃)-5,6-dihydro-4H-pyrrolo[1,2-b]pyrazole. The title compound was prepared in a manner analogous to (R)-3-bromo-5-fluoro-2-(4-fluorophenyl)-5,6-dihydro-4H-pyrrolo[1,2-b]pyrazole (Intermediate 150) Steps A-C; using 4,4-bis(methyl-d₃)pyrrolidine-2-carboxylic acid hydrochloride instead of (2S,4R)-4-fluoropyrrolidine-2-carboxylic acid in Step A; using diphenyl ether instead of xylenes, and heating the reaction mixture in Step A to 200° C. for 3 hours in Step B. MS (ESI): mass calcd. For C₁₄H₈BrD₆FN₂ 314.1; m/z found 314.9 [M+H]⁺.

### Intermediate 154: 3-Bromo-2-(5-fluoropyridin-2-yl)-5,5-bis(methyl-d₃)-5,6-dihydro-4H-pyrrolo[1,2-b]pyrazole

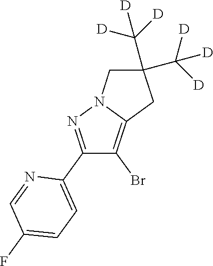 [SMILES: [2H]C([2H])([2H])C1(C([2H])([2H])[2H])Cc2c(Br)c(-c3ccc(F)cn3)nn2C1]

Step A. Di-tert-butyl 4,4-bis(methyl-d₃)-5-oxopyrrolidine-1,2-dicarboxylate: To a cooled solution (−70° C.; dry ice/acetone) of (S)-di-tert-butyl 5-oxopyrrolidine-1,2-dicarboxylate (10.0 g, 35.0 mmol) in anhydrous THF (150 mL) was added LiHMDS (77 mL, 1 M in THF, 77 mmol) dropwise. The resultant mixture was stirred at −70° C. for 1 hour. CD₃I (4.8 mL, 77 mmol) in THF (100 mL) was added dropwise to above solution. The resultant mixture was stirred for 12 hours. The reaction mixture was gradually warmed to room-temperature. The reaction mixture was poured into sat. NH₄Cl (50 mL), and extracted with ethyl acetate (200 mL×3). The combined organic extracts were dried over anhydrous Na₂SO₄, filtered, and concentrated to dryness under reduced pressure. Purification (FCC, SiO₂, eluent: petroleum ether:ethyl acetate=10:1 to 9:1) afforded the title compound (6.9 g, 55% yield, 90% purity) as a yellow solid.

Step B. Di-tert-butyl 4,4-bis(methyl-d₃)pyrrolidine-1,2-dicarboxylate: BH₃·THF (78 mL, 1 M in THF, 78 mmol) was added dropwise to a 0° C. (ice/water) solution of (S)-di-tert-butyl 5-oxo-4,4-bis(tri-D-methyl)pyrrolidine-1,2-dicarboxylate (5.0 g, 16 mmol) in THF (100 mL). The resultant solution was stirred at room-temperature for 16 hours. The reaction mixture was quenched with ice-water (150 mL) at 0° C. and stirred at 0° C. for 0.5 hours. The resultant solution was extracted with ethyl acetate (150 mL×3). The combined organic extracts were washed with brine (150 mL), dried over anhydrous Na₂SO₄, filtered, and concentrated to dryness under reduced pressure. Purification (preparative HPLC using a Phenomenex Genimi NX C18 150 mm×40 mm×5 m column (eluent: 45% to 75% (v/v) CH₃CN and H₂O with 0.225% FA)) afforded the title compound (1.6 g, 33%) as white solid. MS (ESI): mass calcd. for C₁₆H₂₃D₆NO₄ 305.3; m/z; found 193.9 [M−2tBu+H]⁺.

Step C. 4,4-Bis(methyl-d₃)pyrrolidine-2-carboxylic acid hydrochloride. To a solution consisting of (S)-di-tert-butyl 4,4-bis(tri-D-methyl)pyrrolidine-1,2-dicarboxylate (1.6 g, 5.2 mmol) and 1,4-dioxane (10 mL) was added HCl/1,4-dioxane (10 mL, 40 mmol) dropwise. The reaction mixture was stirred at 50° C. for 16 hours. The reaction mixture was concentrated to dryness under reduced pressure to afford the title compound (1.0 g) as a white solid, which was used without further purification.

Step D: 3-Bromo-2-(5-fluoropyridin-2-yl)-5,5-bis(methyl-d₃)-5,6-dihydro-4H-pyrrolo[1,2-b]pyrazole. The title compound was prepared in a manner analogous to (R)-3-bromo-5-fluoro-2-(4-fluorophenyl)-5,6-dihydro-4H-pyrrolo[1,2-b]pyrazole (Intermediate 150) Steps A-C; using 4,4-bis(methyl-d₃)pyrrolidine-2-carboxylic acid hydrochloride instead of (2S,4R)-4-fluoropyrrolidine-2-carboxylic acid in Step A; using 2-ethynyl-5-fluoropyridine instead of 4-fluorophenylacetylene and using diphenyl ether instead of xylenes in Step B. MS (ESI): mass calcd. for C₁₃H₇D₆BrFN₃ 315.1; m/z found 315.9 [M+H]⁺.

### Intermediate 155: 3-Bromo-5,5-difluoro-2-(4-fluorophenyl)-5,6-dihydro-4H-pyrrolo[1,2-b]pyrazole

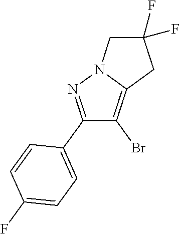 [SMILES: Fc1ccc(-c2nn3c(c2Br)CC(F)(F)C3)cc1]

The title compound was prepared in a manner analogous to (R)-3-bromo-5-fluoro-2-(4-fluorophenyl)-5,6-dihydro-4H-pyrrolo[1,2-b]pyrazole (Intermediate 150) Steps A-C; using 4,4-difluoropyrrolidine-2-carboxylic acid hydrochloride instead of (2S,4R)-4-fluoropyrrolidine-2-carboxylic acid in Step A; and using diphenyl ether instead of xylenes in Step B. MS mass calcd. for C₁₂H₉F₃N₂ 238.1; m/z found 239.1 [M+H]⁺. ¹H NMR (400 MHz, DMSO-d₆) δ 7.87-7.79 (m, 2H), 7.29-7.19 (m, 2H), 6.65 (s, 1H), 4.71 (t, J=13.1 Hz, 2H), 3.62 (t, J=14.3 Hz, 2H).

### Intermediate 156: 3-Bromo-2-(4-fluorophenyl)-4,4-dimethyl-5,6-dihydro-4H-pyrrolo[1,2-b]pyrazole

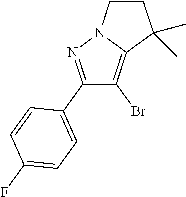 [SMILES: CC1(C)CCn2nc(-c3ccc(F)cc3)c(Br)c21]

The title compound was prepared in a manner analogous to (R)-3-bromo-5-fluoro-2-(4-fluorophenyl)-5,6-dihydro-4H-pyrrolo[1,2-b]pyrazole (Intermediate 150) Steps A-C; using 3,3-dimethylpyrrolidine-2-carboxylic acid hydrochloride instead of (2S,4R)-4-fluoropyrrolidine-2-carboxylic acid in Step A; using diphenyl ether instead of xylenes and heating the reaction mixture to 200° C. for 3 hours in Step B. LC-MS (ESI): mass calcd. For C₁₄H₁₅FN₂ 230.1; m/z found 231.0 [M+H]⁺.

### Intermediate 157: 3′-Bromo-2′-(4-fluorophenyl)-4′H,6′H-spiro[cyclopropane-1,5′-pyrrolo[1,2-b]pyrazole]

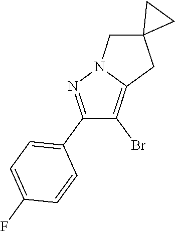 [SMILES: Fc1ccc(-c2nn3c(c2Br)CC2(CC2)C3)cc1]

Step A: (S)-5-Azaspiro[2.4]heptane-6-carboxylic acid. A solution of (S)-5-(tert-butoxycarbonyl)-5-azaspiro[2.4]heptane-6-carboxylic acid (3.0 g, 12 mmol) in HCl/dioxane (20 mL) was stirred at room-temperature for 16 hours. The reaction mixture was concentrated under reduced pressure to afford (2.5 g) of the title compound, which was used in the next step without further purification. ¹H NMR (400 MHz, MeOD) δ 4.63-4.51 (m, 1H), 3.28 (s, 2H), 2.38 (dd, J=8.8, 13.2 Hz, 1H), 2.16 (dd, J=6.6, 13.2 Hz, 1H), 0.85-0.69 (m, 4H).

Step B: 3′-Bromo-2′-(4-fluorophenyl)-4′H,6′H-spiro[cyclopropane-1,5′-pyrrolo[1,2-b]pyrazole]. The title compound was prepared in a manner analogous to (R)-3-bromo-5-fluoro-2-(4-fluorophenyl)-5,6-dihydro-4H-pyrrolo[1,2-b]pyrazole (Intermediate 150) Steps A-C; using (S)-5-azaspiro[2.4]heptane-6-carboxylic acid instead of (2S,4R)-4-fluoropyrrolidine-2-carboxylic acid in Step A; and using diphenyl ether instead of xylenes in Step B. MS (ESI): mass calcd. for C₁₂H₉F₃N₂ 228.1; m/z found 229.0 [M+H]⁺. ¹H NMR (400 MHz, DMSO-d₆) δ 7.84-7.71 (m, 2H), 7.29-7.12 (m, 2H), 6.46 (s, 1H), 4.06-4.04 (m, 2H), 2.87 (s, 2H), 0.90-0.71 (m, 4H).

### Intermediate 158: 3′-Bromo-2′-(5-fluoropyridin-2-yl)-4′,5′-dihydrospiro[cyclopropane-1,6′-pyrrolo[1,2-b]pyrazole]

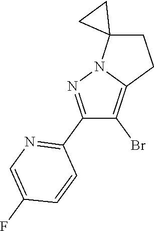 [SMILES: Fc1ccc(-c2nn3c(c2Br)CCC32CC2)nc1]

Step A. 4′,5′-Dihydrospiro[cyclopropane-1,6′-pyrrolo[1,2-c][1,2,3]oxadiazol]-7′-ium-3′-olate. (S)-4-(tert-butoxycarbonyl)-4-azaspiro[2.4]heptane-5-carboxylic acid (1000 mg, 4.14 mmol) was dissolved in TFA and stirred for 2 hours. A solution of sodium nitrite (1430 mg, 20.7 mmol) in water (4 mL) was added to the reaction mixture and the resulting mixture was stirred at r.t. for 1 hour. The reaction mixture was then diluted with water and extracted (3×) with 4:1 chloroform/iPrOH. The combined organic layers were dried (MgSO₄) and concentrated under reduced pressure. TFAA (870 μL, 6.2 mmol) was added dropwise. To a solution of the resulting residue in MeCN (12 mL) was added TFAA (870 μL, 6.2 mmol) dropwise. The reaction mixture was stirred at r.t. for 2 hours To the reaction mixture was added an excess of K₂CO₃ (2.9 g, 20.7 mmol) and stirred for 20 minutes. The solids were filtered, and the resulting filtrated was purified (FCC, SiO₂, 0-100% EtOAc/DCM) to afford (341 mg, 2.24 mmol, 54% yield) of the title compound. MS (ESI): mass calcd. for C₇H₈N₂O₂ 152.1; found 153.1 [M+H]⁺.

Step B. 2′-(5-Fluoropyridin-2-yl)-4′,5′-dihydrospiro[cyclopropane-1,6′-pyrrolo[1,2-b]pyrazole]. A mixture of 4′,5′-dihydrospiro[cyclopropane-1,6′-pyrrolo[1,2-c][1,2,3]oxadiazol]-7′-ium-3′-olate (341 mg, 2.24 mmol), 2-ethynyl-5-fluoropyridine (1360 mg, 11.2 mmol) and xylenes (5 mL) was sparged for 5 minutes with N₂ and heated to 160° C. in a sealed vessel for 8 hours. The mixture was cooled and concentrated under reduced pressure. Purification (FCC, SiO₂, 0-100% EtOAc/hexanes) afforded the title compound. MS (ESI): mass calcd. for C₁₃H₁₂FN₃ 229.1; found 232.2 [M+H]⁺.

Step C. 3′-Bromo-2′-(5-fluoropyridin-2-yl)-4′,5′-dihydrospiro[cyclopropane-1,6′-pyrrolo[1,2-b]pyrazole] A solution of 2′-(5-fluoropyridin-2-yl)-4′,5′-dihydrospiro[cyclopropane-1,6′-pyrrolo[1,2-b]pyrazole] (538 mg, 2.35 mmol) and NBS (418 mg, 2.35 mmol) in DMF (10 mL) was stirred at r.t. for 2 hours. The reaction mixture was concentrated under reduced pressure and the resulting residue was purified (FCC, SiO₂, 0-100% EtOAc/hexanes) to afford (422 mg, 1.37 mmol, 58%) the title compound. MS (ESI): mass calcd. for C₁₃H₁₁BrFN₃ 307.0; found 308.0 [M+H]⁺.

### Intermediate 159: 3′-Bromo-2,2-difluoro-2′-(4-fluorophenyl)-4′,6′-dihydrospiro[cyclopropane-1,5′-pyrrolo[1,2-b]pyrazole]

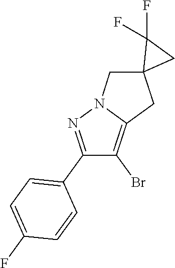 [SMILES: Fc1ccc(-c2nn3c(c2Br)CC2(C3)CC2(F)F)cc1]

Step A. (6S)-5-tert-Butyl 6-methyl 1,1-difluoro-5-azaspiro[2.4]heptane-5,6-dicarboxylate. To a solution consisting of (S)-1-tert-butyl 2-methyl 4-methylenepyrrolidine-1,2-dicarboxylate (5.0 g, 21 mmol), and NaI (6.21 g, 41.4 mmol), in THF (80 mL), was added TMSCF₃ (7.66 g, 53.9 mmol). The reaction mixture was stirred at 70° C. for 16 hours under N₂. The reaction mixture was cooled and quenched with aq. NH₄Cl (80 mL). The reaction mixture was extracted with ethyl acetate (80 mL×3), and the combined organic extracts were dried over anhydrous Na₂SO₄, filtered, and concentrated to dryness under reduced pressure. The resulting residue was purified (FCC, SiO₂, 5-25% EtOAc/petroleum ether) to afford the title compound (5.0 g, 83%) as orange oil. ¹H NMR (400 MHz, CDCl₃): δ 4.55-4.35 (m, 1H), 3.75 (d, J=3.3 Hz, 3H), 3.73-3.42 (m, 2H), 2.60-2.29 (m, 1H), 2.04-1.90 (m, 1H), 1.50-1.40 (m, 9H), 1.40-1.28 (m, 2H).

Step B. (6S)-5-(tert-Butoxycarbonyl)-1,1-difluoro-5-azaspiro[2.4]heptane-6-carboxylic acid. To a solution of (6S)-5-tert-butyl 6-methyl 1,1-difluoro-5-azaspiro[2.4]heptane-5,6-dicarboxylate (5.0 g, 17 mmol) in THF (20 mL), was added a solution of LiOH·H₂O (3.6 g, 86 mmol) in H₂O (10 mL). The reaction mixture was stirred at room-temperature for 2 hours. The reaction mixture was quenched with H₂O (50 mL)) and the aqueous phase was washed with ethyl acetate (50 mL). The pH of the aqueous phase was adjusted to pH=5 with aq. HCl (1 M) and extracted with ethyl acetate (50 mL×3). The combined organic extracts were dried over anhydrous Na₂SO₄, filtered, and concentrated to dryness under reduced pressure to afford the title compound (4.1 g, 86%) as a clear oil. MS (ESI): mass calcd. for C₁₂H₁₇F₂NO₄ 277.11; m/z found 178.1 [M-Boc+H]⁺.

Step C. (6S)-1,1-Difluoro-5-azaspiro[2.4]heptane-6-carboxylic acid hydrochloride: A solution of (65)-5-(tert-butoxycarbonyl)-1,1-difluoro-5-azaspiro[2.4]heptane-6-carboxylic acid (4.1 g, 15 mmol) in HCl/1,4-dioxane (80 mL, 4 M) was stirred at room-temperature for 3 hours. The reaction mixture was concentrated to dryness under reduced pressure afford the title compound (3.0 g) as an oil, which was used in subsequent transformations without additional purification.

Step D: 3′-Bromo-2,2-difluoro-2′-(4-fluorophenyl)-4′,6′-dihydrospiro[cyclopropane-1,5′-pyrrolo[1,2-b]pyrazole]. The title compound was prepared in a manner analogous to (R)-3-bromo-5-fluoro-2-(4-fluorophenyl)-5,6-dihydro-4H-pyrrolo[1,2-b]pyrazole (Intermediate 150) Steps A-C; using 65)-1,1-difluoro-5-azaspiro[2.4]heptane-6-carboxylic acid hydrochloride instead of (2S,4R)-4-fluoropyrrolidine-2-carboxylic acid in Step A; and using diphenyl ether instead of xylenes in Step B. MS (ESI): mass calcd. for C₁₄H₁₀BrF₃N₂ 342.0; m/z found 343.1 [M+H]⁺. ¹H NMR (400 MHz, CDCl₃) δ 7.89-7.81 (m, 2H), 7.19-7.06 (m, 2H), 4.47 (d, J=11.2 Hz, 1H), 4.23 (dd, J=4.0, 11.3 Hz, 1H), 3.29 (d, J=16.6 Hz, 1H), 2.96 (dd, J=3.9, 16.6 Hz, 1H), 1.73-1.60 (m, 2H).

### Intermediate 160: 3′-Bromo-2,2-difluoro-2′-(5-fluoropyridin-2-yl)-4′,6′-dihydrospiro[cyclopropane-1,5′-pyrrolo[1,2-b]pyrazole]

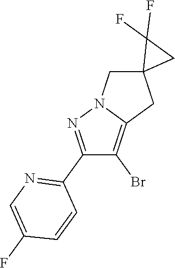 [SMILES: Fc1ccc(-c2nn3c(c2Br)CC2(C3)CC2(F)F)nc1]

The title compound was prepared in a manner analogous to 3′-bromo-2,2-difluoro-2′-(4-fluorophenyl)-4′,6′-dihydrospiro[cyclopropane-1,5′-pyrrolo[1,2-b]pyrazole] (Intermediate 159, Step D); using 2-ethynyl-5-fluoropyridine instead of 4-fluorophenylacetylene. MS (ESI): mass calcd. for C₁₃H₁₀F₃N₃ 265.1; m/z found 266.0 [M+H]⁺.

### Intermediate 161: (4aS,5aS)-3-Bromo-2-(4-fluorophenyl)-4,4a,5,5a-tetrahydrocyclopropa[4,5]pyrrolo[1,2-b]pyrazole

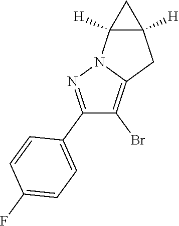 [SMILES: Fc1ccc(-c2nn3c(c2Br)C[C@@H]2C[C@@H]23)cc1]

The title compound was prepared in a manner analogous to 3-bromo-2-(5-fluoropyridin-2-yl)-6,7-dihydro-4H-pyrazolo[5,1-c][1,4]oxazine (Intermediate 37, Steps A-B), except using (4aS,5aS)-4,4a,5,5a-tetrahydrocyclopropa[4,5]pyrrolo[1,2-c][1,2,3]oxadiazol-6-ium-3-olate (Intermediate 8) instead of 6,7-dihydro-4H-[1,2,3]oxadiazolo[4,3-c][1,4]oxazin-8-ium-3-olate (Intermediate 2), using 4-fluorophenylacetylene instead of 2-ethynyl-5-fluoropyridine, using diphenyl ether instead of xylenes, and heating the reaction mixture to 200° C. for 1 hour in Step A. MS (ESI): mass calcd. for C₁₃H₁₀BrFN₂ 292.0; m/z found 292.9 [M+H]⁺.

### Intermediate 162: (4aR,5aR)-3-Bromo-2-(4-fluorophenyl)-4,4a,5,5a-tetrahydrocyclopropa[4,5]pyrrolo[1,2-b]pyrazole

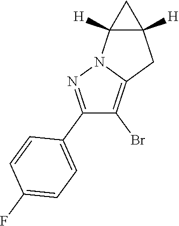 [SMILES: Fc1ccc(-c2nn3c(c2Br)C[C@H]2C[C@H]23)cc1]

The title compound was prepared in a manner analogous to 3-bromo-2-(5-fluoropyridin-2-yl)-6, 7-dihydro-4H-pyrazolo[5,1-c][1,4]oxazine (Intermediate 37, Steps A-B), except using (4aR,5aR)-4,4a,5,5a-tetrahydrocyclopropa[4,5]pyrrolo[1,2-c][1,2,3]oxadiazol-6-ium-3-olate (Intermediate 7) instead of 6,7-dihydro-4H-[1,2,3]oxadiazolo[4,3-c][1,4]oxazin-8-ium-3-olate (Intermediate 2), using 4-fluorophenylacetylene instead of 2-ethynyl-5-fluoropyridine, using diphenyl ether instead of xylenes, and heating the reaction mixture to 200° C. for 1 hour. MS (ESI): mass calcd. for C₁₉H₁₄FN₅ 331.1; m/z found 332.0 [M+H]⁺. ¹H NMR (400 MHz, DMSO-d₆) δ 7.81-7.71 (m, 2H), 7.25-7.14 (m, 2H), 6.38 (s, 1H), 4.08 (t, J=5.5 Hz, 1H), 3.11 (dd, J=6.6, 16.9 Hz, 1H), 2.89 (d, J=16.8 Hz, 1H), 2.27-2.16 (m, 1H), 1.15-1.04 (m, 1H), 0.39-0.30 (m, 1H).

### Intermediate 163: Racemic (3bS,4aR)-3-Bromo-2-(4-fluorophenyl)-3b,4,4a,5-tetrahydrocyclopropa[3,4]pyrrolo[1,2-b]pyrazole

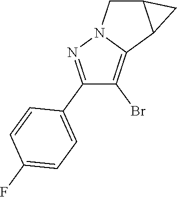 [SMILES: Fc1ccc(-c2nn3c(c2Br)C2CC2C3)cc1]

The title compound was prepared in a manner analogous to 3-bromo-2-(5-fluoropyridin-2-yl)-6,7-dihydro-4H-pyrazolo[5,1-c][1,4]oxazine (Intermediate 37, Steps A-B), except using racemic (3bS,4aR)-3b,4,4a,5-tetrahydrocyclopropa[3,4]pyrrolo[1,2-c][1,2,3]oxadiazol-6-ium-3-olate (Intermediate 6) instead of 6,7-dihydro-4H-[1,2,3]oxadiazolo[4,3-c][1,4]oxazin-8-ium-3-olate (Intermediate 2), using 4-fluorophenylacetylene instead of 2-ethynyl-5-fluoropyridine, using diphenyl ether instead of xylenes, and heating the reaction mixture to 200° C. for 1 hour. MS (ESI): mass calcd. for C₁₃H₁₀BrFN₂ 292.0; m/z found 293.0 [M+H]⁺.

### Intermediate 164: 7-Bromo-6-(5-fluoropyridin-2-yl)-2,2-dimethyl-2,3-dihydropyrazolo[5,1-b]oxazole

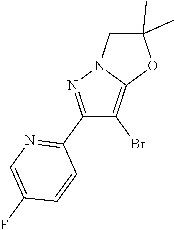 [SMILES: CC1(C)Cn2nc(-c3ccc(F)cn3)c(Br)c2O1]

Step A. 1-Hydrazineyl-2-methylpropan-2-ol. A solution of 2,2-dimethyldioxirane (1 g, 13.9 mmol), and hydrazine hydrate (2.45 g, 41.6 mmol) in ethanol (10 mL) was stirred at 60° C. for 4 hours. The reaction mixture was concentrated to dryness to afford the title compound. The resulting residue was used directly in the next step without purification.

Step B. 3-(5-Fluoropyridin-2-yl)-1-(2-hydroxy-2-methylpropyl)-1H-pyrazol-5-ol: A solution of methyl 3-(5-fluoropyridin-2-yl)-3-oxopropanoate (Intermediate 119 Step B, 1 g, 5.07 mmol) and 1-hydrazinyl-2-methylpropan-2-ol (2.38 g, 22.9 mmol) in AcOH (10 mL) was heated at 95° C. for 5 hours. The reaction mixture was cooled and concentrated under reduced pressure. Purification (FCC, SiO₂, 0-100% EtOAc/petroleum ether) afforded the title compound (1.1 g, 70% purity, 3.07 mmol, 60% yield). MS (ESI): mass calcd. for C₁₂H₁₄FN₃O₂ 251.1; found, 252.1 [M+H]⁺.

Step C. 6-(5-Fluoropyridin-2-yl)-2,2-dimethyl-2,3-dihydropyrazolo[5,1-b]oxazole. A solution of 3-(5-fluoropyridin-2-yl)-1-(2-hydroxy-2-methylpropyl)-1H-pyrazol-5-ol (1.1 g, 4.38 mmol) in PPA (10 mL) was stirred at 120° C. for 6 hours. The reaction mixture was then cooled and quenched with sat. aq. NaHCO₃(100 mL). The resulting mixture was extracted with EtOAc 3×, and the combined organics were dried (Na₂SO₄), filtered, and concentrated under reduced pressure. Purification (FCC, SiO₂, 0-100% EtOAc/petroleum ether) afforded the title compound (550 mg, 88% purity, 2.08 mmol, 47% yield).

Step D. 7-Bromo-6-(5-fluoropyridin-2-yl)-2,2-dimethyl-2,3-dihydropyrazolo[5,1-b]oxazole. To a solution consisting of 6-(5-fluoropyridin-2-yl)-2,2-dimethyl-2,3-dihydropyrazolo[5,1-b]oxazole (500 mg, 2.14 mmol) in DCM (5 mL), was added NBS (570 mg, 3.20 mmol). The reaction mixture was stirred at room-temperature for 16 hours. The reaction mixture was diluted with water, extracted with DCM (3×), and the combined organics were dried (Na₂SO₄), filtered, and concentrated under reduced pressure. Purification of the resulting residue (FCC, SiO₂, 0-100% EtOAc/petroleum ether) afforded the title compound (350 mg, 24% yield). MS (ESI): mass calcd. for C₁₂H₁₁BrFN₃O 311.0; found, 312.0 [M+H]⁺.

### Intermediate 165: 3-Bromo-2-(5-fluoropyridin-2-yl)-6-(methoxymethyl)-4,5,6,7-tetrahydropyrazolo[1,5-a]pyridine

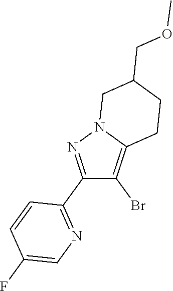 [SMILES: COCC1CCc2c(Br)c(-c3ccc(F)cn3)nn2C1]

Step A. (2-(5-Fluoropyridin-2-yl)-4,5,6,7-tetrahydropyrazolo[1,5-a]pyridin-6-yl)methanol. To a cooled (0° C.) solution of methyl 2-(5-fluoropyridin-2-yl)-4,5,6,7-tetrahydropyrazolo[1,5-a]pyridine-6-carboxylate (Intermediate 58, 500 mg, 1.82 mmol) in THF (15 mL) was added LiBH₄ (0.514 g, 23.6 mmol) portionwise The reaction mixture was stirred at room-temperature for 16 hours. The reaction mixture was poured into water (30 mL), and extracted with ethyl acetate (50 mL×3). The combined organic extracts were dried over anhydrous Na₂SO₄, filtered, and concentrated to dryness under reduced pressure to give the title compound (405 mg, 90%) as a yellow solid. MS (ESI): mass calcd. for C₁₃H₁₄FN₃O 247.1 m/z found 248.0 [M+H]⁺.

Step B. 2-(5-Fluoropyridin-2-yl)-6-(methoxymethyl)-4,5,6,7-tetrahydropyrazolo[1,5-a]pyridine. To a cooled (0° C.) solution of (2-(5-fluoropyridin-2-yl)-4,5,6,7-tetrahydropyrazolo[1,5-a]pyridin-6-yl)methanol (405 mg, 1.64 mmol) in THF (20 mL), was added NaH (131 mg, 60% purity, 3.28 mmol) portionwise. The reaction mixture was stirred at 0° C. (ice/water) for 0.5 h. Iodomethane (2.33 g, 16.4 mmol) was added dropwise to the reaction mixture. The reaction mixture was stirred for 3 hours and gradually warmed to room-temperature. The reaction mixture was quenched with sat. NaHCO₃(15 mL). The reaction mixture was extracted with ethyl acetate (30 mL×3), and the combined organic extracts were washed with brine (20 mL×2), dried over anhydrous Na₂SO₄, filtered, and concentrated to dryness under reduced pressure. The resulting residue was purified (FCC, SiO₂, petroleum ether:ethyl acetate=1:0 to 1:1) to afford the title compound (375 mg, 87%) as colorless oil. MS (ESI): mass calcd. for C₁₄H₁₆FN₃O 261.1; m/z found 261.9 [M+H]⁺.

Step C. 3-Bromo-2-(5-fluoropyridin-2-yl)-6-(methoxymethyl)-4,5,6,7-tetrahydropyrazolo[1,5-a]pyridine. To a solution of 2-(5-fluoropyridin-2-yl)-6-(methoxymethyl)-4,5,6,7-tetrahydropyrazolo[1,5-a]pyridine (325 mg, 1.24 mmol) in DMF (10 mL) was added NBS (244 mg, 1.37 mmol). The reaction mixture was stirred at 25° C. for 3 hours. The reaction mixture was combined with an additional batch of the same reaction mixture and concentrated to dryness under reduced pressure. The resulting residue was purified (FCC, SiO₂, eluent: petroleum ether:ethyl acetate=10:1 to 1:3) to afford the title compound (443 mg). MS (ESI): mass calcd. for C₁₄H₁₅BrFN₃O 339.0; m/z found 339.7 [M+H]⁺.

### Intermediate 166: 3-Bromo-2-(5-fluoropyridin-2-yl)-6-(2-methoxyethyl)-6-methyl-4,5,6,7-tetrahydropyrazolo[1,5-a]pyridine

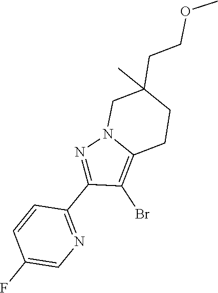 [SMILES: COCCC1(C)CCc2c(Br)c(-c3ccc(F)cn3)nn2C1]

Step A. Methyl 2-(5-fluoropyridin-2-yl)-6-(2-methoxyethyl)-4,5,6,7-tetrahydropyrazolo[1,5-a]pyridine-6-carboxylate. To a cooled (−70° C.) solution of methyl 2-(5-fluoropyridin-2-yl)-4,5,6,7-tetrahydropyrazolo[1,5-a]pyridine-6-carboxylate (Intermediate 58, 2.30 g, 8.36 mmol) in THF (30 mL) was added LiHMDS (20.9 mL, 1 M in THF, 20.9 mmol) dropwise. The reaction mixture was stirred at −70° C. for 1 h. 1-Bromo-2-methoxyethane (7.85 mL, 83.5 mmol) was added to the reaction mixture at −70° C. The mixture was stirred at 20° C. for 16 h. The reaction mixture was quenched with H₂O (25 mL) and extracted with ethyl acetate (70 mL×3). The combined organic extracts were dried over anhydrous Na₂SO₄, filtered, and concentrated to dryness under reduced pressure. The resulting residue was purified (FCC, SiO₂, (10-100% EtOAc/petroleum ether) to afford the title compound (850 mg, 28%) as a yellow solid. MS (ESI): mass calcd. for C₁₇H₂₀FN₃O₃ 333.2 m/z found 334.0 [M+H]⁺.

Step B. (2-(5-Fluoropyridin-2-yl)-6-(2-methoxyethyl)-4,5,6,7-tetrahydropyrazolo[1,5-a]pyridin-6-yl)methanol. To a cooled solution (0° C.) of methyl 2-(5-fluoropyridin-2-yl)-6-(2-methoxyethyl)-4,5,6,7-tetrahydropyrazolo[1,5-a]pyridine-6-carboxylate (50 mg, 0.15 mmol) in THF (2 mL) was added LiBH₄ (49.0 mg, 2.25 mmol). The reaction mixture was stirred at room-temperature for 16 hours. The reaction mixture was quenched with H₂O (3 mL) and extracted with ethyl acetate (10 mL×3). The combined organic extracts were dried Na₂SO₄, filtered, and concentrated to dryness under reduced pressure to afford the title compound (35 mg) as a yellow solid, which was used in the next step without further purification. MS (ESI): mass calcd. for C₁₆H₂₀FN₃O₂ 305.2; m/z found 306.1 [M+H]⁺.

Step C. (2-(5-Fluoropyridin-2-yl)-6-(2-methoxyethyl)-4,5,6,7-tetrahydropyrazolo[1,5-a]pyridin-6-yl)methyl methanesulfonate. To a cooled (0° C.) solution of (2-(5-fluoropyridin-2-yl)-6-(2-methoxyethyl)-4,5,6,7-tetrahydropyrazolo[1,5-a]pyridin-6-yl)methanol (380 mg, 1.24 mmol), and Et₃N (0.520 mL, 3.73 mmol), in dichloromethane (10 mL) was added MsCl (1.73 g, 15.1 mmol) portion-wise. The reaction mixture was stirred at room-temperature under N₂ for 3 hours. The reaction mixture was quenched with sat. NaHCO₃(15 mL) and extracted with dichloromethane (40 mL×3). The combined organic extracts were dried over anhydrous Na₂SO₄, filtered, and concentrated to dryness under reduced pressure. The resulting residue was purified (FCC, SiO₂, 0-17% EtOAc/petroleum ether) to afford the title compound (450 mg) as a yellow solid. MS (ESI): mass calcd. for C₁₇H₂₂FN₃O₄S 383.1; m/z found 384.0 [M+H]⁺.

Step D. 2-(5-Fluoropyridin-2-yl)-6-(2-methoxyethyl)-6-methyl-4,5,6,7-tetrahydropyrazolo[1,5-a]pyridine. To a solution of (2-(5-fluoropyridin-2-yl)-6-(oxetan-3-ylmethyl)-4,5,6,7-tetrahydropyrazolo[1,5-a]pyridin-6-yl)methyl methanesulfonate (370 mg, 0.965 mmol), Zn (335 mg, 5.12 mmol), and HMPA (10 mL) was added NaI (391 mg, 2.61 mmol). The reaction mixture was stirred for 60 hours at 125° C. The reaction mixture was cooled, then quenched with H₂O (20 mL). The reaction mixture was extracted with ethyl acetate (50 mL×3) and the combined organic extracts were dried over anhydrous Na₂SO₄, filtered, and concentrated to dryness under reduced pressure. Purification of the resulting residue (FCC, SiO₂, 0-17% EtOAc/petroleum ether) afforded the title compound (210 mg, 66%) as a yellow solid. MS (ESI): mass calcd. for C₁₆H₂₀FN₃O 289.2; m/z found 289.9 [M+H]⁺.

Step E. 3-Bromo-2-(5-fluoropyridin-2-yl)-6-(2-methoxyethyl)-6-methyl-4,5,6,7-tetrahydropyrazolo[1,5-a]pyridine. To a cooled (0° C.) solution of 2-(5-fluoropyridin-2-yl)-6-(2-methoxyethyl)-6-methyl-4,5,6,7-tetrahydropyrazolo[1,5-a]pyridine (131 mg, 0.453 mmol) in dichloromethane (5 mL) was added NBS (88.6 mg, 0.498 mol). The reaction mixture was stirred at room-temperature for 2 hours. The reaction mixture was poured into H₂O (1 mL) and extracted with dichloromethane (5 mL×3). The combined organic extracts were concentrated to dryness under reduced pressure. The resulting residue was purified (FCC, SiO₂, 10-25% EtOAc/petroleum ether) to afford the title compound (90 mg, 48%) as a yellow solid. MS (ESI): mass calcd. for C₁₆H₁₉BrFN₃O 367.1; m/z found 367.9 [M+H]⁺.

### Intermediate 167: Racemic 3-Bromo-2-(5-fluoropyridin-2-yl)-6-methyl-6-(oxetan-3-ylmethyl)-4,5,6,7-tetrahydropyrazolo[1,5-a]pyridine

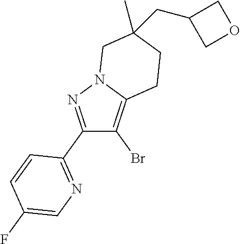 [SMILES: CC1(CC2COC2)CCc2c(Br)c(-c3ccc(F)cn3)nn2C1]

The title compound was prepared in a manner analogous to 3-bromo-2-(5-fluoropyridin-2-yl)-6-(2-methoxyethyl)-6-methyl-4,5,6,7-tetrahydropyrazolo[1,5-a]pyridine (Intermediate 166, Steps A-E), except using 3-(iodomethyl)oxetane instead of 1-bromo-2-methoxyethane in Step A. MS (ESI): mass calcd. for C₁₇H₁₉BrFN₃O 379.1; m/z found 379.9 [M+H]⁺.

### Intermediate 168: 3-Bromo-2-(5-fluoropyridin-2-yl)-6,6-bis(methyl-d₃)-4,5,6,7-tetrahydropyrazolo[1,5-a]pyridine

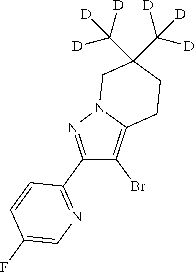 [SMILES: [2H]C([2H])([2H])C1(C([2H])([2H])[2H])CCc2c(Br)c(-c3ccc(F)cn3)nn2C1]

Step A. 3-Phenyltetrahydro-1H-oxazolo[3,4-a]pyridin-5(3H)-one. 4-Methylbenzenesulfonic acid To a cooled solution (0° C.) of 6-(hydroxymethyl)piperidin-2-one (15.0 g, 116 mmol), and benzaldehyde (27.2 mL, 268 mmol), in toluene (450 mL) was added (340 mg, 1.97 mmol). The reaction mixture was stirred at 130° C. for 72 hours. The reaction mixture was cooled and concentrated to dryness under reduced pressure. The resulting residue was purified (FCC, SiO₂, 10-50% EtOAc/petroleum ether) to afford the title compound (23 g, 82%) as a white solid. MS (ESI): mass calcd. for C₁₃H₁₅NO₂ 217.1; m/z found 217.9 [M+H]⁺.

Step B. 3-Phenyl-6-(methyl-d₃)tetrahydro-1H-oxazolo[3,4-a]pyridin-5(3H)-one. To a cooled (−70° C.) solution of diisopropylamine (7.90 mL, 56.4 mmol) in THF (15 mL) was added n-BuLi (25.0 mL, 62.5 mmol) dropwise. The reaction mixture was stirred at −70° C. for 30 mins. A solution of 3-phenyltetrahydro-1H-oxazolo[3,4-a]pyridin-5(3H)-one (6.80 g, 31.3 mmol) in THF (15 mL) was added to the reaction mixture at −70° C. The reaction mixture was stirred at −70° C. for 60 mins. CD₃I (3.89 mL, 62.5 mmol) was added to the reaction mixture at −70° C. The reaction mixture was stirred for an additional 5 mins at −70° C., then allowed to stir while warming to room-temperature over 2 hours. The reaction mixture was quenched with H₂O (30 mL) and extracted with ethyl acetate (100 mL×3). The combined organic extracts were concentrated to dryness under reduced pressure. The resulting residue was purified (FCC, SiO₂, 10-25% EtOAc/petroleum ether) to afford the title compound (6.0 g, 79%) as a yellow solid. MS (ESI): mass calcd. for C₁₄H₁₄D₃NO₂ 234.1; m/z found 235.0 [M+H]⁺.

Step C. 3-Phenyl-6,6-bis-(methyl-d₃)tetrahydro-1H-oxazolo[3,4-a]pyridin-5(3H)-one. To a cooled (−70° C.) solution of di-isopropylamine (3.59 mL, 25.6 mmol) in THF (10 mL) was added n-BuLi (11.3 mL, 2.5 M in THF, 28.3 mmol) dropwise. The reaction mixture was stirred at −70° C. for 0.5 h. 3-Phenyl-6-(methyl-d₃)tetrahydro-1H-oxazolo[3,4-a]pyridin-5(3H)-one (3.00 g, 12.8 mmol) in THF (10 mL) was added the reaction mixture solution at −70° C. The reaction mixture was stirred at −70° C. for 1 h. CD₃I (1.59 mL, 25.6 mmol) was added the reaction mixture and the reaction mixture was stirred at 20° C. for 16 h. The reaction mixture was quenched with H₂O (10 mL) and extracted with ethyl acetate (70 mL×3). The combined organic extracts were dried over anhydrous Na₂SO₄, filtered, and concentrated to dryness under reduced pressure. The resulting residue was purified by FCC (SiO₂, 10-50% EtOAc/petroleum ether) to afford the title compound (2.0 g, 53%) as a yellow oil. MS (ESI): mass calcd. for C₁₅H₁₃D₆NO₂ 251.2; m/z found 252.0 [M+H]⁺.

Step D. 6-(Hydroxymethyl)-3,3-bis(methyl-d₃)piperidin-2-one. To a cooled (0° C.) solution of 3-phenyl-6,6-bis(methyl-d₃)tetrahydro-1H-oxazolo[3,4-a]pyridin-5(3H)-one (1.20 g, 4.77 mmol) in dichloromethane (8 mL) was added TFA (8 mL). The reaction mixture was stirred at room-temperature for 16 hours. The reaction mixture was concentrated to dryness under reduced pressure. The resulting residue was purified (preparative HPLC using a Phenomenex Luna C18 150×30 mm×5 μm column (eluent: 10% to 30% (v/v) CH₃CN and H₂O with (0.225% HCOOH)) to afford the title compound (600 mg) as a yellow oil. MS (ESI): mass calcd. for C₈H₉D₆NO₂ 163.2; m/z found 163.9 [M+H]⁺.

Step E. (5,5-Bis(methyl-d₃)piperidin-2-yl)methanol. To a cooled (0° C.) solution of 6-(hydroxymethyl)-3,3-bis(methyl-d₃) piperidin-2-one (600 mg, 3.68 mmol) in THF (10 mL) was added LiAlH₄ (697 mg, 18.4 mmol) in portions. The reaction mixture was stirred at 65° C. for 16 hours. The reaction mixture was cooled to room-temperature, and quenched with NaOH (aq. 15%, 10 mL). The resulting reaction mixture was stirred at room-temperature for 30 mins, then filtered through Celite®. The filter cake was washed with EtOAc (20 mL), and the filtrate was concentrated to dryness under reduced pressure to give the title compound (450 mg) as a yellow solid, which was used in the next step without further purification.

Step F. Benzyl 2-(hydroxymethyl)-5,5-bis(methyl-d₃)piperidine-1-carboxylate. To a solution consisting of (5,5-bis(methyl-d₃)piperidin-2-yl)methanol (450 mg, 3.02 mmol), and K₂CO₃ (1.25 g, 9.04 mmol), in THF: H₂O (1:1, 10 mL) was added CbzCl (771 mg, 4.52 mmol). The resultant mixture was stirred for 16 hours at room-temperature under N₂. The reaction mixture was quenched with H₂O (15 mL) and extracted with ethyl acetate (50 mL×3). The combined organic extracts were dried over anhydrous Na₂SO₄, filtered, and concentrated to dryness under reduced pressure. Purification of the resulting residue, FCC (SiO₂, 10-20% EtOAc/petroleum ether) afforded the title compound (450 mg, 53%) as a yellow oil. MS (ESI): mass calcd. for C₁₆H₁₇D₆NO₃ 283.2; m/z found 284.0 [M+H]⁺.

Step G. 1-((Benzyloxy)carbonyl)-5,5-bis(methyl-d₃)piperidine-2-carboxylic acid. To a cooled (0° C.) solution of CrO₃ (635 mg, 6.35 mmol) in H₂O (1.40 mL) was added H₂SO₄ (0.500 mL). The reaction mixture was stirred at 0° C. for 0.5 h. The resultant solution was added to a cooled (0° C.) solution of benzyl 2-(hydroxymethyl)-5,5-bis(methyl-d₃)piperidine-1-carboxylate (450 mg, 1.59 mmol) in acetone (25 mL). The reaction mixture was stirred at 20° C. for 0.5 h. The reaction mixture was quenched with i-propanol (10 mL) and extracted with ethyl acetate (50 mL×3). The combined organic extracts were dried over anhydrous Na₂SO₄, filtered, and concentrated to dryness under reduced pressure to afford the title compound (460 mg, crude) as a yellow oil, which was used in the next step without further purification. MS (ESI): mass calcd. for C₁₆H₁₅D₆NO₄ 297.2; m/z found 320.0 [M+Na]⁺.

Step H. 5,5-Bis(methyl-d₃)piperidine-2-carboxylic acid. 1-((Benzyloxy)carbonyl)-5,5-bis(methyl-d₃)piperidine-2-carboxylic acid (200 mg, 0.673 mmol), 10% Pd/C (100 mg), and MeOH (5 mL) were added to a 50 mL hydrogenation bottle. The resultant mixture was stirred under H₂ (15 psi) at room-temperature for 16 hours. The suspension was filtered through a pad of Celite® and the pad washed with ethyl acetate (10 mL). The filtrate was concentrated to dryness under reduced pressure to afford the title compound (110 mg), which was used in the next step without further purification.

Step I: 3-Bromo-2-(5-fluoropyridin-2-yl)-6,6-bis(methyl-d₃)-4,5,6,7-tetrahydropyrazolo[1,5-a]pyridine. The title compound was prepared in a manner analogous to Intermediate 150 Steps A-C; using 5,5-bis(methyl-d₃)piperidine-2-carboxylic acid instead of (2S,4R)-4-fluoropyrrolidine-2-carboxylic acid in Step A; and using 2-ethynyl-5-fluoropyridine instead of 4-fluorophenylacetylene in Step B. MS (ESI): mass calcd. for C₁₄H₉BrD₆FN₃ 329.1; m/z found 329.9 [M+H]⁺.

### Intermediate 169: 3-Bromo-6-(fluoromethyl-d₂)-2-(5-fluoropyridin-2-yl)-6-(methyl-d₃)-4,5,6,7-tetrahydropyrazolo[1,5-a]pyridine

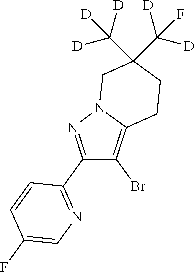 [SMILES: [2H]C([2H])([2H])C1(C([2H])([2H])F)CCc2c(Br)c(-c3ccc(F)cn3)nn2C1]

Step A: (2-(5-Fluoropyridin-2-yl)-6-(methyl-d₃)-4,5,6,7-tetrahydropyrazolo[1,5-a]pyridin-6-yl)methan-d₂-ol. The title compound was prepared in a manner analogous to Intermediate 59; except using CD₃I instead of methyl iodide and LiAlD₄ instead of LiBH₄. MS (ESI): mass calcd. for C₁₄H₁₁D₅FN₃O 266.2 m/z found 267.9 [M+H]⁺.

Step B. (2-(5-Fluoropyridin-2-yl)-6-(methyl-d₃)-4,5,6,7-tetrahydropyrazolo[1,5-a]pyridin-6-yl)methyl-d₂ methanesulfonate. To a cooled (0° C.) solution of (2-(5-fluoropyridin-2-yl)-6-(methyl-d₃)-4,5,6,7-tetrahydropyrazolo[1,5-a]pyridin-6-yl)methan-d₂-ol (2.00 g, 7.51 mmol), Et₃N (5.76 mL, 41.3 mmol), in dichloromethane (20 mL) was added MsCl (11.4 g, 99.5 mmol) dropwise. The reaction mixture was stirred at room-temperature under N₂ for 5 hours. The reaction mixture was quenched with sat. NaHCO₃(10 mL) and extracted with dichloromethane (35 mL×3). The combined organic extracts were dried over anhydrous Na₂SO₄, filtered, and concentrated to dryness under reduced pressure. The resulting residue was purified by FCC (SiO₂, 0-100% EtOAc/petroleum ether) to afford the title compound (750 mg, 29%) as a yellow oil. MS (ESI): mass calcd. for C₁₅H₁₃D₅FN₃₀₃S 344.1; m/z found 345.9 [M+H]⁺. ¹H NMR (400 MHz, DMSO-d₆): δ 8.54 (s, 1H), 7.95-7.90 (m, 1H), 7.79-7.67 (m, 1H), 6.57 (s, 1H), 4.05-3.90 (m, 2H), 3.22 (s, 3H), 2.90-2.78 (m, 2H), 1.86-1.69 (m, 2H).

Step C. 6-(Fluoromethyl-d₂)-2-(5-fluoropyridin-2-yl)-6-(methyl-d₃)-4,5,6,7-tetrahydropyrazolo[1,5-a]pyridine. To a solution of (2-(5-fluoropyridin-2-yl)-6-(methyl-d₃)-4,5,6,7-tetrahydropyrazolo[1,5-a]pyridin-6-yl)methyl-d₂ methanesulfonate (750 mg, 2.18 mmol) in methyl ethyl ketone (15 mL) was added tetrabutylammonium fluoride trihydrate (3.80 g, 12.0 mmol). The reaction mixture was stirred for 48 hours at 90° C. The reaction mixture was cooled and quenched with sat. NH₄Cl (15 mL) and extracted with ethyl acetate (30 mL×3). The combined organic extracts were dried over anhydrous Na₂SO₄, filtered, and concentrated to dryness under reduced pressure. The resulting residue was purified by FCC (SiO₂,0-100% EtOAc/petroleum ether) to afford the compound (190 mg, 32%) as a yellow solid. MS (ESI): mass calcd. for C₁₄H₁₀D₅F₂N₃ 268.2; m/z found 269.9 [M+H]⁺.

Step D. 3-Bromo-6-(fluoromethyl-d₂)-2-(5-fluoropyridin-2-yl)-6-(methyl-d₃)-4,5,6,7-tetrahydropyrazolo[1,5-a]pyridine. To a solution consisting of 6-(fluoromethyl-d₂)-2-(5-fluoropyridin-2-yl)-6-(methyl-d₃)-4,5,6,7-tetrahydropyrazolo[1,5-a]pyridine (190 mg, 0.708 mmol) in dichloromethane (5 mL) was added NBS (139 mg, 0.781 mmol). The reaction mixture was stirred at 25° C. for 3 hours. The reaction mixture was concentrated to dryness under reduced pressure. The resulting residue was purified by FCC (SiO₂, 10-75% EtOAc/petroleum ether) to afford the title compound (245 mg, 100%). MS (ESI): mass calcd. for C₁₄H₉BrD₅F₂N₃ 346.1; m/z found 347.1 [M+H]⁺.

### Intermediate 170: 3-Bromo-2-(5-fluoropyridin-2-yl)-6-(methyl-d₃)-4,5,6,7-tetrahydropyrazolo[1,5-a]pyridine-6-carbonitrile

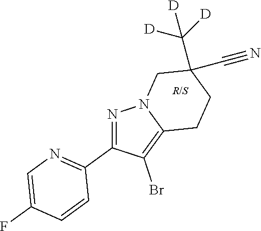 [SMILES: [2H]C([2H])([2H])C1(C#N)CCc2c(Br)c(-c3ccc(F)cn3)nn2C1]

Step A: Methyl 2-(5-fluoropyridin-2-yl)-6-(methyl-d₃)-4,5,6,7-tetrahydropyrazolo[1,5-a]pyridine-6-carboxylate. To a cooled solution, −78° C., of methyl 2-(5-fluoropyridin-2-yl)-4,5,6,7-tetrahydropyrazolo[1,5-a]pyridine-6-carboxylate (Intermediate 58, 615 mg, 2.234 mmol) in THF (9 mL), under nitrogen atmosphere, was added lithium bis(trimethylsilyl)amide (3.35 mL, 3.35 mmol) dropwise over 10 minutes. After the reaction mixture was stirred for 50 min. Iodomethane-d₃ (0.417 mL, 6.702 mmol) was added dropwise to the reaction mixture, then the reaction mixture was allowed to warm to ambient temperature and stirred overnight. The reaction mixture was diluted with water and extracted with ethyl acetate. The combined organic layers were dried over Na₂SO₄, filtered, and concentrated under reduced pressure. Purification of the resulting residue by FCC (SiO₂, Hex/EtOAc) afforded the title compound as a white solid. (571 mg, 87%). MS (ESI): mass calcd. for C₁₅H₁₃D₃FN₃O₂ 292.1; m/z found 293.2 [M+H]⁺.

Step B: (2-(5-Fluoropyridin-2-yl)-6-(methyl-d₃)-4,5,6,7-tetrahydropyrazolo[1,5-a]pyridin-6-yl)methanol. To a cooled solution, −78° C., of methyl 2-(5-fluoropyridin-2-yl)-6-(methyl-d₃)-4,5,6,7-tetrahydropyrazolo[1,5-a]pyridine-6-carboxylate (571 mg, 1.953 mmol) in THF (8 mL), nitrogen atmosphere, was added lithium aluminum hydride (2M in THF, 1.32 mL, 2.64 mmol) dropwise over 10 minutes. The reaction mixture was stirred −78° C. for 45 minutes. The reaction mixture was allowed to warm to ambient temperature over the course of 1 hour. EtOAc (15 mL) was added very slowly to quench excess lithium aluminum hydride. The reaction mixture was stirred for 30 minutes, followed by the addition of saturated aqueous Rochell's salt solution (25 mL). The resulting reaction mixture was stirred overnight at room temperature. The reaction mixture was extracted with EtOAc several times, and the combined organic layers were dried over Na₂SO₄, filtered, and concentrated under reduced pressure. Purification of the resulting residue by FCC (SiO₂, Hex/10% MeOH in EtOAc) afforded the title compound a white solid (458 mg, 89%). MS (ESI): mass calcd. for C₁₄H₁₃D₃FN₃O 264.1; m/z found 265.2 [M+H]⁺.

Step C: 2-(5-Fluoropyridin-2-yl)-6-(methyl-d₃)-4,5,6,7-tetrahydropyrazolo[1,5-a]pyridine-6-carbaldehyde. A solution of (2-(5-fluoropyridin-2-yl)-6-(methyl-d₃)-4,5,6,7-tetrahydropyrazolo[1,5-a]pyridin-6-yl)methanol (488 mg, 1.846 mmol) in DCM (5 mL) and Dess-Martin periodinane (2.4 g, 5.66 mmol) was stirred at room temperature for 4 hours. Water was added to the reaction mixture, and the resulting solution was extracted with DCM several times. The combined organic layers were dried over Na₂SO₄, filtered, and concentrated under reduced pressure. Purification of the resulting residue by FCC (SiO₂, Hex/EtOAc) afforded the title compound as an off white yellow solid (238 mg, 49%). MS (ESI): mass calcd. for C₁₄H₁₁D₃FN₃O, 262.1; m/z found 263.1 [M+H]⁺.

Step D: (E)-2-(5-Fluoropyridin-2-yl)-6-(methyl-d₃)-4,5,6,7-tetrahydropyrazolo[1,5-a]pyridine-6-carbaldehyde oxime. A solution of 2-(5-fluoropyridin-2-yl)-6-(methyl-d₃)-4,5,6,7-tetrahydropyrazolo[1,5-a]pyridine-6-carbaldehyde (238 mg, 0.907 mmol) in THF (3 mL) and hydroxylamine hydrochloride (127 mg, 1.815 mmol) and sodium acetate (223 mg, 2.722 mmol) were heated 50° C. for 5 hours. The reaction mixture solids we filtered off and washed with copious amounts of EtOAc. The resulting filtrate was concentrated under reduced pressure to afford the title compound as a white solid (251 mg, 99%) which was used without further purification. MS (ESI): mass calcd. for C₁₄H₁₂D₃FN₄O 277.1; m/z found 278.1 [M+H]⁺.

Step E: 2-(5-Fluoropyridin-2-yl)-6-(methyl-d₃)-4,5,6,7-tetrahydropyrazolo[1,5-a]pyridine-6-carbonitrile. To a cooled (−10° C.) solution of (E)-2-(5-fluoropyridin-2-yl)-6-(methyl-d₃)-4,5,6,7-tetrahydropyrazolo[1,5-a]pyridine-6-carbaldehyde oxime (251 mg, 0.905 mmol) in dry THF (15 mL) under nitrogen atmosphere was added thionyl chloride (1 mL, 13.6 mmol) dropwise. The reaction mixture was stirred at −10° C. for 1 hour. The reaction mixture was allowed to warm to room temperature and stirred at room temperature for 30 minutes. The reaction mixture was cooled to 0° C. and TEA (1.25 mL, 9 mmol) was added dropwise. The reaction mixture was allowed to warm to room temperature and stirred for an additional 2 hours. The reaction mixture was quenched first with water, followed by saturated aqueous NaHCO₃. The reaction mixture was extracted with EtOAc several times and the combined organic layers were dried over Na₂SO₄, filtered, and concentrated under reduced pressure. The resulting residue was purified by FCC (SiO₂, Hex/10% MeOH in EtOAc) to afford the title compound as an off white yellow solid (143 mg, 61%). MS (ESI): mass calcd. for C₁₄H₁₀D₃FN₄ 259.1; m/z found 260.1 [M+H]⁺.

Step F: 3-Bromo-2-(5-fluoropyridin-2-yl)-6-(methyl-d₃)-4,5,6,7-tetrahydropyrazolo[1,5-a]pyridine-6-carbonitrile. To a cooled (0° C.) solution of 2-(5-fluoropyridin-2-yl)-6-(methyl-d₃)-4,5,6,7-tetrahydropyrazolo[1,5-a]pyridine-6-carbonitrile (143 mg, 0.551 mmol) in DMF (1.5 mL) was added N-bromosuccinimide (106 mg, 0.596 mmol) in a few portions. The reaction mixture was allowed to warm to room temperature. The reaction mixture was diluted with water and extracted with EtOAc several times. The combined organic layers were dried over Na₂SO₄, filtered, and concentrated under reduced pressure. The resulting residue was purified by FCC (SiO₂, Hex/10% MeOH in EtOAc) to afford the compound as a white solid (153 mg, 82%). MS (ESI): mass calcd. for C₁₄H₉D₃BrF₄, 337.0; m/z found 338.1 [M+H]⁺.

### Intermediate 171: 3-Bromo-6-fluoro-2-(5-fluoropyridin-2-yl)-6-((methoxy-d₃)methyl)-4,5,6,7-tetrahydropyrazolo[1,5-a]pyridine

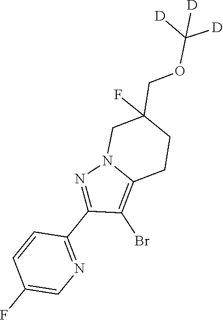 [SMILES: [2H]C([2H])([2H])OCC1(F)CCc2c(Br)c(-c3ccc(F)cn3)nn2C1]

The title compound was prepared in a manner analogous to 3-bromo-6-fluoro-2-(5-fluoropyridin-2-yl)-6-(methoxymethyl)-4,5,6,7-tetrahydropyrazolo[1,5-a]pyridine (Intermediate 61, Steps A-D), except using CD₃I instead of methyl iodide in Step C. MS (ESI): mass calcd. for C₁₄H₁₁BrD₃F₂N₃O 360.1; m/z found 360.8 [M+H]⁺.

### Intermediate 172: 3-Bromo-6-fluoro-2-(5-fluoropyridin-2-yl)-6-((methoxy-d₃)methyl-d₂)-4,5,6,7-tetrahydropyrazolo[1,5-a]pyridine

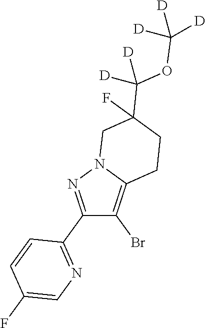 [SMILES: [2H]C([2H])([2H])OC([2H])([2H])C1(F)CCc2c(Br)c(-c3ccc(F)cn3)nn2C1]

The title compound was prepared in a manner analogous to 3-bromo-6-fluoro-2-(5-fluoropyridin-2-yl)-6-(methoxymethyl)-4,5,6,7-tetrahydropyrazolo[1,5-a]pyridine (Intermediate 61), Steps A-D, except using LiAlD₄ instead of LiBH₄ in Step B; and CD₃I instead of methyl iodide in Step C. MS (ESI): mass calcd. for C₁₄H₉BrD₅F₂N₃O 362.1; m/z found 362.9 [M+H]⁺.

### Intermediate 173: 3-Bromo-6-((difluoromethoxy)methyl)-6-fluoro-2-(5-fluoropyridin-2-yl)-4,5,6,7-tetrahydropyrazolo[1,5-a]pyridine

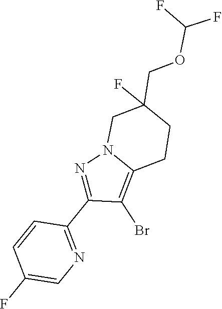 [SMILES: Fc1ccc(-c2nn3c(c2Br)CCC(F)(COC(F)F)C3)nc1]

Step A. 6-((Difluoromethoxy)methyl)-6-fluoro-2-(5-fluoropyridin-2-yl)-4,5,6,7-tetrahydropyrazolo[1,5-a]pyridine. To a solution of (6-fluoro-2-(5-fluoropyridin-2-yl)-4,5,6,7-tetrahydropyrazolo[1,5-a]pyridin-6-yl)methanol (Intermediate 61 Step B, 360 mg, 1.36 mmol), and CuI (51.7 mg, 271 μmol), in CH₃CN (5 mL) under N₂, was added 2,2-difluoro-2-(fluorosulfonyl)acetic acid (0.421 mL, 4.07 mmol) slowly, the reaction mixture was heated to 60° C. and stirred at 60° C. for 30 min before being cooled to room-temperature. The reaction mixture was quenched with sat. NaHCO₃(5 mL) and extracted with ethyl acetate (20 mL×3). The combined organic extracts were dried over anhydrous Na₂SO₄, filtered, and concentrated to dryness under reduced pressure. The resulting residue was purified by FCC (SiO₂, 10-20% EtOAc/petroleum ether) to afford the title compound (130 mg, 30%) as a yellow solid. MS (ESI): mass calcd. for C₁₄H₁₃F₄N₃O 315.10; m/z found 316.3 [M+H]⁺. ¹H NMR (400 MHz, DMSO-d₆) δ 8.55 (d, J=2.9 Hz, 1H), 7.95-7.92 (m, 1H), 7.77-7.72 (m, 1H), 7.15-6.63 (m, 1H), 6.60 (s, 1H), 4.47-4.30 (m, 2H), 4.27-4.15 (m, 2H), 3.04-2.98 (m, 1H), 2.92-2.75 (m, 1H), 2.31-2.20 (m, 1H), 2.14-1.91 (m, 1H).

Step B. 3-Bromo-6-((difluoromethoxy)methyl)-6-fluoro-2-(5-fluoropyridin-2-yl)-4,5,6,7-tetrahydropyrazolo[1,5-a]pyridine: To a cooled, 0° C., solution of 6-((difluoromethoxy)methyl)-6-fluoro-2-(5-fluoropyridin-2-yl)-4,5,6,7-tetrahydropyrazolo[1,5-a]pyridine (130 mg, 0.412 mmol) in dichloromethane (5 mL) was added NBS (80.7 mg, 0.453 mol). The reaction mixture was stirred at room-temperature for 2 hours. The reaction mixture was poured into H₂O (3 mL) and extracted with dichloromethane (15 mL×3). The combined organic layers were concentrated to dryness under reduced pressure. The resulting residue was purified by FCC (SiO₂, eluent: petroleum ether:ethyl acetate=10:1 to 1:3) to afford the title compound (137 mg, 84%) as a yellow solid. MS (ESI): mass calcd. for C₁₄H₁₂BrF₄N₃O 393.0; m/z found 393.9 [M+H]⁺.

### Intermediate 174: 3-Bromo-6-fluoro-2-(5-fluoropyridin-2-yl)-6-((trifluoromethoxy)methyl)-4,5,6,7-tetrahydropyrazolo[1,5-a]pyridine

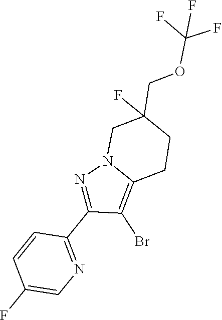 [SMILES: Fc1ccc(-c2nn3c(c2Br)CCC(F)(COC(F)(F)F)C3)nc1]

Step A. O-((6-Fluoro-2-(5-fluoropyridin-2-yl)-4,5,6,7-tetrahydropyrazolo[1,5-a]pyridin-6-yl)methyl)S-methyl carbonodithioate. To a cooled solution (0° C.) of (6-fluoro-2-(5-fluoropyridin-2-yl)-4,5,6,7-tetrahydropyrazolo[1,5-a]pyridin-6-yl)methanol (Intermediate 61 product from Step B, 330 mg, 1.24 mmol) in THF (3 mL) was added NaH (124 mg, 60% purity, 3.10 mmol). The reaction mixture was stirred at 20° C. under N₂ for 1 hour, then cooled to 0° C. CS₂ (0.374 mL. 6.22 mmol) was added to the cooled reaction mixture, and the reaction mixture was stirred at 20° C. under N₂ for 2 hours. The reaction mixture was cooled to 0° C., Mel (0.155 mL. 2.49 mmol) was added and the reaction mixture was stirred at room-temperature under N₂ for 2 hours. The reaction mixture was quenched with H₂O (5 mL) and extracted with ethyl acetate (20 mL×2). The combined organic extracts were concentrated to dryness under reduced pressure. The resulting residue was purified by FCC (SiO₂, 10-50% EtOAc/petroleum ether) to afford product (310 mg, 69%) as a yellow solid. MS (ESI): mass calcd. for C₁₅H₁₅F₂N₃OS₂ 355.1; m/z found 356.0 [M+H]⁺.

Step B. 3-Bromo-6-fluoro-2-(5-fluoropyridin-2-yl)-6-((trifluoromethoxy)methyl)-4,5,6,7-tetrahydropyrazolo[1,5-a]pyridine. To a cooled solution (−70° C.) of 1,3-dibromo-5,5-dimethylimidazolidine-2,4-dione (847 mg, 2.96 mmol) in dichloromethane (2 mL) was added HF-pyridine (3.80 mL, 29.5 mmol). The reaction mixture was stirred at −70° C. for 10 min. A solution of O-((6-fluoro-2-(5-fluoropyridin-2-yl)-4,5,6,7-tetrahydropyrazolo[1,5-a]pyridin-6-yl)methyl)S-methyl carbonodithioate (350 mg, 0.985 mmol) in dichloromethane (3 mL) was added to the above reaction mixture. The reaction mixture was stirred at −70° C. for 1 h. The reaction mixture was stirred at 20° C. for 4 h. The reaction mixture was quenched with aq. Na₂SO₃ (3 mL) and extracted with dichloromethane (15 mL×3). The combined organic extracts were dried over Na₂SO₄, filtered, and concentrated to dryness under reduced pressure. The resulting residue was purified by FCC (SiO₂, eluent: petroleum ether:ethyl acetate=10:1 to 1:2) to afford the title compound (270 mg, 63%) as a yellow solid. MS (ESI): mass calcd. for C₁₄H₁₁BrF₅N₃O 411.0; m/z found 411.9 [M+H]⁺.

### Intermediate 175: 3′-Bromo-2,2-difluoro-2′-(4-fluorophenyl)-4′,5′-dihydro-7′H-spiro[cyclopropane-1,6′-pyrazolo[1,5-a]pyridine]

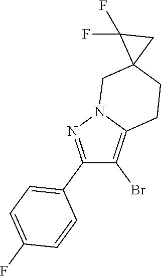 [SMILES: Fc1ccc(-c2nn3c(c2Br)CCC2(C3)CC2(F)F)cc1]

Step A. 1-tert-Butyl 2-methyl 5-methylenepiperidine-1,2-dicarboxylate. To a cooled solution (0° C.) of methyltriphenylphosphonium bromide (10 g, 28.0 mmol) in toluene (100 mL) under nitrogen, was added KHMDS (29 mL, 1 M solution in THF, 29 mmol) dropwise. The reaction mixture was warmed to room-temperature and stirred for 2 hours. A solution of 1-tert-butyl 2-methyl 5-oxopiperidine-1, 2-dicarboxylate (5 g, 19.4 mmol) in toluene (50 mL) was added dropwise to the above solution at 0° C. The reaction mixture was stirred this temperature for 4 hours. Then the reaction mixture was allowed to warm to room-temperature and stirred for 14 hours. The reaction mixture was quenched with sat. NH₄Cl (80 mL) and extracted with ethyl acetate (50 mL×3). The combined organic extracts were washed with brine (100 mL), dried over Na₂SO₄, filtered, and concentrated under reduced pressure. The resulting residue was purified by FCC (SiO₂, 0-10% EtOAc/petroleum ether) to afford the title product (2.46 g, 45%) as colorless oil. MS mass calcd. for C₁₃H₂₁NO₄ 255.2; m/z found 155.9 [M-Boc+H]⁺. ¹H NMR (400 MHz, CDCl₃): δ 4.99-4.68 (m, 3H), 4.49-4.23 (m, 1H), 3.81-3.56 (m, 4H), 2.34-2.06 (m, 3H), 1.80 (s, 1H), 1.50-1.38 (m, 9H).

Step B. 5-tert-Butyl 6-methyl 1,1-difluoro-5-azaspiro[2.5]octane-5,6-dicarboxylate. NaI (722 mg, 4.82 mmol) was added to a solution consisting of 1-tert-butyl 2-ethyl 5-methylenepiperidine-1, 2-dicarboxylate (2.46 g, 9.64 mmol) in dry THF (30 mL). The reaction mixture was heated to 70° C. TMSCF₃ (4.9 mL, 33.4 mmol) was added to above reaction mixture and the reaction mixture was stirred at 70° C. for 4 hours. The reaction mixture was cooled to room-temperature and quenched with sat. sodium thiosulfate (40 mL) and diluted with ethyl acetate (30 mL). The reaction mixture was separated and the aqueous phase was extracted with ethyl acetate (30 mL×2). The combined organic extracts were washed with brine (50 mL), dried over Na₂SO₄, filtered, and concentrated under reduced pressure. The resulting residue was purified by FCC (SiO₂, 0-17% EtOAc/petroleum ether) to afford the title compound (3.1 g, 97%) as a yellow oil. MS (ESI): mass calcd. For C₁₄H₂₁F₂NO₄ 305.1 m/z found 205.8 [M-Boc+H]⁺. ¹H NMR (400 MHz, CDCl₃): δ 5.11-4.76 (m, 1H), 3.86-3.52 (m, 4H), 3.47-3.15 (m, 1H), 2.39-2.21 (m, 1H), 1.95-1.57 (m, 3H), 1.47 (s, 9H), 1.37-1.30 (m, 2H).

Step C. 5-(tert-Butoxycarbonyl)-1,1-difluoro-5-azaspiro[2.5]octane-6-carboxylic acid. NaOH (1.4 g, 35.0 mmol) was added to a solution of 5-tert-butyl 6-methyl 1, 1-difluoro-5-azaspiro[2.5]octane-5,6-dicarboxylate (3.1 g, 10.2 mmol), MeOH (30 mL), and H₂O (6 mL). The reaction mixture was stirred at room-temperature for 2 hours. The reaction mixture was concentrated under reduced pressure. The resulting residue was diluted with H₂O (20 mL), and the pH was adjusted with conc. HCl (37%) to pH=5. The resulting mixture was extracted with ethyl acetate (20 mL×2). The combined organic extracts were dried over Na₂SO₄, filtered, and concentrated under reduced pressure to afford the title compound (2.8 g) as yellow oil. MS (ESI): mass calcd. for C₁₃H₁₉F₂NO₄ 291.1; m/z found 192.0 [M-Boc+H]⁺.

Step D. 1,1-Difluoro-5-azaspiro[2.5]octane-6-carboxylic acid hydrochloride. HCl/1,4-dioxane (15 mL, 60 mmol, 4 M) was added to a solution of 5-(tert-butoxycarbonyl)-1,1-difluoro-5-azaspiro[2.5]octane-6-carboxylic acid (2.8 g, crude) in dichloromethane (20 mL). The reaction mixture was stirred at room-temperature for 1 hour. The reaction mixture was concentrated under reduced pressure to afford the title compound (2.6 g) as a white solid. MS mass calcd. for C₈H₁₁F₂NO₂·HCl 191.1; m/z found 191.8 [M+H]⁺.

Step E: 3′-Bromo-2,2-difluoro-2′-(4-fluorophenyl)-4′,5′-dihydro-7′H-spiro[cyclopropane-1,6′-pyrazolo[1,5-a]pyridine]. The title compound was prepared in a manner analogous to (R)-3-bromo-5-fluoro-2-(4-fluorophenyl)-5,6-dihydro-4H-pyrrolo[1,2-b]pyrazole (Intermediate 150) Steps A-C; using 1,1-difluoro-5-azaspiro[2.5]octane-6-carboxylic acid hydrochloride instead of (2S,4R)-4-fluoropyrrolidine-2-carboxylic acid in Step A; and using oxydibenzene instead of xylenes, in Step B. MS (ESI): mass calcd. for C₁₅H₁₂BrF₃N₂ 356.0; m/z found 356.7 [M+H]⁺.

### Intermediate 176: 3′-Bromo-2,2-difluoro-2′-(5-fluoropyridin-2-yl)-5′, 7′-dihydro-4′H-spiro[cyclopropane-1,6′-pyrazolo[1,5-a]pyridine]

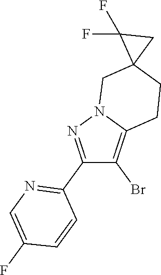 [SMILES: Fc1ccc(-c2nn3c(c2Br)CCC2(C3)CC2(F)F)nc1]

The title compound was prepared in a manner analogous to 3′-bromo-2,2-difluoro-2′-(4-fluorophenyl)-4′,5′-dihydro-7′H-spiro[cyclopropane-1,6′-pyrazolo[1,5-a]pyridine] (Intermediate 175, Steps A-E) except using 2-ethynyl-5-fluoropyridine instead of 4-fluorophenylacetylene in Step E. MS (ESI): mass calc'd. for C₁₄H₁₂F₃N₃ 279.1; m/z, found 280.3 [M+H]⁺. ¹H NMR (400 MHz, CDCl₃): δ 8.47 (d, J=2.8 Hz, 1H), 7.95-7.76 (m, 1H), 7.49-7.36 (m, 1H), 6.61 (s, 1H), 4.36-4.05 (m, 2H), 3.03-2.90 (m, 2H), 2.01 (t, J=6.4 Hz, 2H), 1.48-1.37 (m, 2H).

### Intermediate 177: Racemic 3-Bromo-5,5-difluoro-2-(4-fluorophenyl)-4a,5,5a,6-tetrahydro-4H-cyclopropa[d]pyrazolo[1,5-a]pyridine

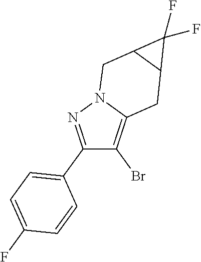 [SMILES: Fc1ccc(-c2nn3c(c2Br)CC2C(C3)C2(F)F)cc1]

Step A: 3-(tert-Butyl) 4-methyl 7,7-difluoro-3-azabicyclo[4.1.0]heptane-3,4-dicarboxylate. A vial was charged with 1-(tert-butyl) 2-methyl 3,6-dihydropyridine-1,2(2H)-dicarboxylate (500 mg, 2.072 mmol), sodium iodide (112 mg, 0.747 mmol), and THF (4 mL). The vial was sealed, and heated to 70° C., and trimethyl(trifluoromethyl)silane (150 μL, 0.001 mmol) was added every 30 minutes over a 4 hour period after which the reaction was incomplete. The reaction was cooled to r.t. overnight and then heated to 70° C. in the morning and trimethyl(trifluoromethyl)silane (150 μL, 0.001 mmol) was added every 30 minutes over a 6 hour period. The reaction mixture was partitioned between DCM/water and the aqueous layer was extracted with DCM (X2). The combined organics were concentrated under reduced pressure. The resulting residue was purified by FCC (SiO₂, 0-100% EA/hexanes) to afford the title compound. MS (ESI): mass calcd. for C₁₃H₁₉F₂NO₄ 291.1; m/z found 314.1 [M+Na]⁺.

Step B: 3-(tert-Butoxycarbonyl)-7,7-difluoro-3-azabicyclo[4.1.0]heptane-4-carboxylic acid. A solution of 3-(tert-butyl) 4-methyl 7,7-difluoro-3-azabicyclo[4.1.0]heptane-3,4-dicarboxylate (185 mg, 0.635 mmol), 4N aq. NaOH sol'n (2 mL, 8 mmol), in MeOH (5 mL) was stirred at r.t. for 2 hours, after which the mixture was concentrated to remove MeOH. The aqueous layer was washed with Et₂₀ (3×). The aqueous layer was then acidified to ˜pH1 and extracted with 20% iPrOH/DCM (4×). The organics were combined, dried with MgSO₄, filtered, and concentrated under reduced pressure to afford the title compound which was used directly in the next step. MS (ESI): mass calcd. for C₁₂H₁₇F₂NO₄ 277.1; m/z found 300.1 [M+Na]⁺.

Step C: Racemic 5,5-Difluoro-4a,5,5a,6-tetrahydro-4H-cyclopropa[d][1,2,3]oxadiazolo[3,4-a]pyridin-7-ium-3-olate. A solution of 3-(tert-butoxycarbonyl)-7,7-difluoro-3-azabicyclo[4.1.0]heptane-4-carboxylic acid (127 mg, 0.458 mmol) in TFA (1 mL) was stirred for 30 min. The reaction mixture was concentrated under reduced pressure. The resulting residue was dissolved in water (0.44 mL) and HCl (37% in H₂O, 0.08 mL). To the resulting reaction mixture was added sodium nitrite (47 mg, 0.69 mmol) in a single portion, and the reaction mixture was stirred at r.t. for 2 hours. The reaction was diluted with H₂O and extracted with 20% iPrOH/CHCl₃ (3×). The combined organic layers were dried with MgSO₄, filtered, and concentrated under high-vacuum. The resulting residue was dissolved in MeCN (1.3 mL) and TFAA (0.1 mL, 0.69 mmol) was added dropwise. The reaction mixture was stirred at r.t. for two hours, the reaction was quenched with K₂CO₃. After 20 minutes the reaction mixture was concentrated to remove MeCN. The resulting reaction mixture was partitioned between water and 20% iPrOH/DCM, and the aqueous layer was extracted three times with 20% iPrOH/DCM. The combined organics were dried (MgSO₄), filtered, and concentrated under reduced pressure to afford the title compound. MS (ESI): mass calcd. for C₇H₆F₂N₂O₄ 188.0; m/z found 189.1 [M+H]⁺.

Step D: Racemic 3-Bromo-5,5-difluoro-2-(4-fluorophenyl)-4a,5,5a,6-tetrahydro-4H-cyclopropa[d]pyrazolo[1,5-a]pyridine. The title compound was prepared in a manner analogous to (R)-3-bromo-5-fluoro-2-(4-fluorophenyl)-5,6-dihydro-4H-pyrrolo[1,2-b]pyrazole (Intermediate 150) Steps A-C; using 3-(tert-butoxycarbonyl)-7,7-difluoro-3-azabicyclo[4.1.0]heptane-4-carboxylic acid instead of (2S,4R)-4-fluoropyrrolidine-2-carboxylic acid in Step A. MS (ESI): mass calcd. for C₁₄H₁₀BrF₃N₂ 342.0; m/z found 343.0 [M+H]⁺.

### Intermediate 178: Racemic 3-Bromo-2-(4-fluorophenyl)-5,5a,6,6a-tetrahydro-4H-cyclopropa[e]pyrazolo[1,5-a]pyridine

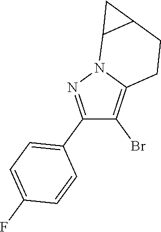 [SMILES: Fc1ccc(-c2nn3c(c2Br)CCC2CC23)cc1]

The title compound was prepared in a manner analogous to 3-bromo-2-(5-fluoropyridin-2-yl)-6,7-dihydro-4H-pyrazolo[5,1-c][1,4]oxazine (Intermediate 37, Steps A-B), except using racemic (5aR,6aS)-5,5a,6,6a-tetrahydro-4H-cyclopropa[e][1,2,3]oxadiazolo[3,4-a]pyridin-7-ium-3-olate (Intermediate 14) instead of 6,7-dihydro-4H-[1,2,3]oxadiazolo[4,3-c][1,4]oxazin-8-ium-3-olate (Intermediate 2) and 1-ethynyl-4-fluorobenzene instead of 2-ethynyl-5-fluoropyridine in Step A. MS (ESI): mass calcd. for C₁₃H₁₁BrN₃ 306.0; m/z found 307.0 [M+H]⁺.

### Intermediate 179: (5a*R,6a*S)-3-Bromo-2-(5-fluoropyridin-2-yl)-5,5a,6,6a-tetrahydro-4H-cyclopropa[e]pyrazolo[1,5-a]pyridine

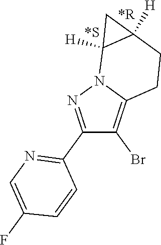 [SMILES: Fc1ccc(-c2nn3c(c2Br)CC[C@@H]2C[C@@H]23)nc1]

Step A: 6-Oxopiperidine-2-carboxylic acid. To a solution of 6-oxo-1,6-dihydropyridine-2-carboxylic acid (50.0 g, 359 mmol, 1.00 eq) in MeOH (1.50 L) was added Pd/C (13.0 g, 10% purity) under N₂. The reaction mixture was degassed under vacuum and purged with H₂ several times. The reaction mixture was stirred at 20° C. under H₂ (50 psi) for 12 h. The reaction mixture was filtered. The resulting filtrate was concentrated under reduced pressure to afford the title compound (380 g, 2.58 mol, 90% yield, 97% purity) as a white solid. MS (ESI): mass calcd. for C₆H₉NO₃ 143.1; m/z found 144.1 [M+H]⁺. ¹H NMR (400 MHz, CD₃OD): δ 2.47 (t, J=6.00 Hz, 1H), 0.78-0.67 (m, 2H), 0.48-0.47 (m, 1H), 0.30-0.18 (m, 1H), 0.17-0.15 (m, 2H).

Step B: Ethyl 6-oxopiperidine-2-carboxylate. SOCl₂ (283 g, 2.39 mol, 173 mL, 1.10 eq) was added dropwise to a cooled (−5° C.) solution of EtOH (3.20 L). To the reaction mixture was added 6-oxopiperidine-2-carboxylic acid (320 g, 2.17 mol, 97% purity, 1.00 eq) at 0° C. The reaction mixture was stirred at 20° C. for 6 h. The mixture was concentrated under reduced pressure. The residue resulting residue was dissolved in toluene (3.20 L). To the reaction mixture was added Et₃N (464 g, 4.59 mol, 638 mL, 2.12 eq) at 20° C. The mixture was stirred at 20° C. for 0.5 h. The mixture was filtered. The filtrate was concentrated to give the title compound (381 g) as yellow oil. MS (ESI): mass calcd. for C₈H₁₃NO₃ 171.1; m/z found 172.2 [M+H]⁺. ¹H NMR (400 MHz, CDCl₃): δ 6.70 (s, 1H), 4.20-4.17 (m, 2H), 4.16-4.03 (m, 1H), 2.34-2.31 (m, 2H), 2.31-2.29 (m, 1H), 1.83-1.76 (m, 3H), 1.31-1.17 (m, 3H).

Step C: 1-(tert-Butyl) 2-ethyl 6-oxopiperidine-1,2-dicarboxylate. To a solution of ethyl 6-oxopiperidine-2-carboxylate (380 g, 2.00 mol, 90.0% purity, 1.00 eq) in toluene (3.60 L) was added DMAP (12.2 g, 99.8 mmol, 0.05 eq) and Boc₂O (654 g, 3.00 mol, 688 mL, 1.50 eq) at 20° C. The reaction mixture was stirred at 20° C. for 12 h. The mixture was concentrated under reduced pressure. The resulting residue was purified (FCC, SiO₂, eluted with petroleum ether/ethyl acetate=4/1) to afford the title compound (380 g, 1.36 mol, 68.0% yield, 97.0% purity) as yellow oil. MS (ESI): mass calcd. for C₁₃H₂₁NO₅ 271.1; m/z found 172.2 [M+H]⁺. ¹H NMR (400 MHz, CDCl₃): δ 4.64-4.62 (m, 1H), 4.20-4.15 (m, 2H), 2.56-2.43 (m, 2H), 2.10-1.98 (m, 1H), 1.98-1.74 (m, 1H), 1.74-1.72 (m, 2H), 1.44 (s, 9H), 1.25-1.18 (m, 3H).

Step D: 1-(tert-Butyl) 2-ethyl 3,4-dihydropyridine-1,2(2H)-dicarboxylate. To a solution of 1-(tert-butyl) 2-ethyl 6-oxopiperidine-1,2-dicarboxylate (189 g, 675 mmol, 97% purity, 1.00 eq) in toluene (1.90 L) was added LiEt₃BH (1.00 M, 743 mL, 1.10 eq) at −50° C. The reaction mixture was stirred at −50° C. for 0.5 h. To the reaction mixture was added DIPEA (375 g, 2.91 mol, 506 mL, 4.30 eq), TFAA (212 g, 1.01 mol, 140 mL, 1.50 eq), and DMAP (1.24 g, 10.1 mmol, 0.02 eq) at −50° C. The reaction mixture was warmed to 20° C., and stirred at 20° C. for 2.5 h. The reaction mixture was quenched with water (2.00 L). The organic layer was separated and washed with water (2.00 L), dried over Na₂SO₄, filtered, and concentrated under reduced pressure. The resulting residue was purified (FCC, SiO₂, eluted with petroleum ether/ethyl acetate=100/1) to afford the title compound (303 g, 1.16 mol, 86% yield, 98% purity) as yellow oil. MS (ESI): mass calcd. for C₁₃H₂₁NO₄ 255.2; m/z found 156.2 [M+H]⁺. ¹H NMR (400 MHz, CDCl₃): δ 6.89-6.75 (m, 1H), 4.88-4.77 (m, 2H), 4.20-4.10 (m, 2H), 2.34-2.28 (m, 1H), 2.01-1.86 (m, 3H), 1.47-1.41 (m, 9H), 1.25-1.20 (m, 3H).

Step E: 2-(tert-Butyl) 3-ethyl 2-azabicyclo[4.1.0]heptane-2,3-dicarboxylate. To a solution of 1-(tert-butyl) 2-ethyl 3,4-dihydropyridine-1,2(2H)-dicarboxylate (50.0 g, 191 mmol, 98% purity, 1.00 eq) in toluene (1.00 L) was added Et₂Zn (1.00 M, 575 mL, 3.00 eq) and a solution of CH₂I₂ (308 g, 1.15 mol, 92.9 mL, 6.00 eq) in toluene (100 mL) under N₂ at −30° C. The reaction mixture was stirred at −15° C. for 16 h. The reaction mixture was quenched with saturated NaHCO₃ solution (2.00 L), filtered, and the organic layer was separated. The organic layer was washed with brine (2.00 L), dried over Na₂SO₄, filtered and concentrated under reduced pressure. The resulting residue was purified by FCC (SiO₂, eluted with petroleum ether/ethyl acetate=100/5) to afford the title compound (95.2 g, 307 mmol, 80% yield, 87% purity) as yellow oil. MS (ESI): mass calcd. for C₁₄H₂₃NO₄ 269.3; m/z found 170.3 [M+H]⁺. ¹H NMR (400 MHz, CDCl₃): δ 4.48-4.01 (m, 3H), 2.89-2.79 (m, 1H), 2.08-1.40 (m, 3H), 1.35-1.34 (m, 9H), 1.21-1.05 (m, 5H), 0.76-0.69 (m, 1H), 0.42-0.13 (m, 1H).

Step F: 2-(tert-Butoxycarbonyl)-2-azabicyclo[4.1.0]heptane-3-carboxylic acid. To a mixture of 2-(tert-butyl) 3-ethyl 2-azabicyclo[4.1.0]heptane-2,3-dicarboxylate (95.2 g, 307 mmol, 87% purity, 1.00 eq) in EtOH (900 mL) was added NaOH (2.00 M, 615 mL, 4.00 eq) at 0° C. The reaction mixture was stirred at 20° C. for 48 h. The reaction mixture was concentrated under reduced pressure. The resulting residue was dissolved in water (2.00 L). The pH was adjusted to 4-5 by progressively adding AcOH. The reaction mixture was extracted with dichloromethane (1.00 L*3). The combined organic layers were washed with brine (2.00 L), dried over Na₂SO₄, filtered, and concentrated under reduced pressure to afford the title compound (94.0 g) as yellow oil. MS (ESI): mass calcd. for C₁₂H₁₉NO₄ 241.1; m/z found 142.3 [M+H]⁺. ¹H NMR (400 MHz, CDCl₃): δ 10.1 (s, 1H), 4.61-4.08 (m, 1H), 2.96-2.85 (m, 1H), 2.09-1.86 (m, 2H), 1.68-1.67 (m, 1H), 1.67-1.64 (m, 1H), 1.48-1.42 (m, 9H), 1.25-1.81 (m, 1H), 0.83-0.81 (m, 1H), 0.24-0.22 (m, 1H).

Step G: 5,5a,6,6a-Tetrahydro-4H-cyclopropa[e][1,2,3]oxadiazolo[3,4-a]pyridin-7-ium-3-olate. A mixture of 2-(tert-butoxycarbonyl)-2-azabicyclo[4.1.0]heptane-3-carboxylic acid (82.0 g, 339 mmol, 1.00 eq) in TFA (561 g, 4.93 mol, 364 mL, 14.5 eq) was stirred at 20° C. for 0.5 h. The reaction mixture was concentrated under reduced pressure. The resulting residue was dissolved in H₂O (314 mL) and HCl (66.9 g, 679 mmol, 65.6 mL, 37.0% purity, 2.00 eq). To the reaction mixture was added NaNO₂ (35.1 g, 509 mmol, 1.50 eq) at 20° C. The reaction mixture was stirred at 20° C. for 4 h. The reaction mixture was diluted with water (100 mL) and extracted with methanol/dichoromethane=1/4(100 mL*3). The combined organic layers were dried over Na₂SO₄, filtered, and concentrated under reduced pressure. The resulting residue was dissolved in MeCN (840 mL). To the reaction mixture was added TFAA (428 g, 2.04 mol, 283 mL, 6.00 eq) at 20° C. The mixture was stirred at 20° C. for 12 h. The reaction mixture was quenched with a 2M K₂CO₃ solution (200 mL) and extracted with methanol/dichoromethane=1/4 (100 mL*3). The combined organic layers were dried over Na₂SO₄, filtered, and concentrated under reduced pressure to afford the title compound (18.0 g) as brown oil. MS (ESI): mass calcd. for C₇H₈N₂O₂ 152.1; m/z found 153.3 [M+H]⁺.

Step H: 5-Fluoro-2-((trimethylsilyl)ethynyl)pyridine. To a solution of 2-bromo-5-fluoropyridine (185 g, 1.05 mol, 1.00 eq) in THF (1.85 L) was added ethynyltrimethylsilane (206 g, 2.10 mol, 291 mL, 2.00 eq), Pd(PPh₃)₂Cl₂ (36.8 g, 52.5 mmol, 0.05 eq) and CuI (40.0 g, 210 mmol, 0.20 eq) at 20° C. The reaction mixture was purged 3 times with N₂. To the mixture was added DIEA (271 g, 2.10 mol, 366 mL, 2.00 eq) at 20° C. The reaction mixture was heated to 60° C. for 2 h. The reaction mixture was quenched with water (1.50 L) and extracted with dichoromethane (2.00 L*2). The combined organic layers were washed with brine (3.00 L), dried over Na₂SO₄, filtered, and concentrated under reduced pressure. The resulting residue was purified (FCC, SiO₂, eluted with petroleum ether/ethyl acetate=1/0) to afford the title compound (240 g, 1.21 mol, 58% yield, 98% purity) as brown oil. MS (ESI): mass calcd. for C₁₀H₁₂FNSi 193.1; m/z found 194.2 [M+H]⁺. ¹H NMR (400 MHz, DMSO) δ 8.54 (d, J=2.00 Hz, 1H), 7.76-7.71 (m, 1H), 7.64-7.60 (m, 1H), 0.23 (s, 9H).

Step I: 2-Ethynyl-5-fluoropyridine. A mixture of 5-fluoro-2-((trimethylsilyl)ethynyl)pyridine (72.0 g, 363 mmol, 97.6% purity, 1.00 eq) and KOH (1.30 M, 279 mL, 1.00 eq) in xylene (150 mL) was stirred at 20° C. for 4 h. The mixture was separated. The organic layer was washed with brine (50 mL), dried over Na₂SO₄, and filtered to afford a solution of the title compound (44 g) in xylene which was used to the next step without further purification. MS (ESI): mass calcd. for C₇H₄FN 121.0; m/z found 122.2 [M+H]⁺.

Step J: 2-(5-Fluoropyridin-2-yl)-5,5a,6,6a-tetrahydro-4H-cyclopropa[e]pyrazolo[1,5-a]pyridine. A mixture of 5,5a,6,6a-tetrahydro-4H-cyclopropa[e][1,2,3]oxadiazolo[3,4-a]pyridin-7-ium-3-olate (18.0 g, 118 mmol, 1.00 eq) and 2-ethynyl-5-fluoropyridine (42.9 g, 354 mmol, 3.00 eq) in xylene (50.0 mL) was stirred at 150° C. for 12 h. The mixture was concentrated. The resulting residue was purified (FCC, SiO₂, eluted with petroleum ether/ethyl acetate=10/1) to afford the title compound (10.0 g, 31.4 mmol, 27% yield, 97% purity) as brown oil. MS (ESI): mass calcd. for C₁₃H₁₂FN₃ 229.1; m/z found 230.2 [M+H]⁺. ¹H NMR (400 MHz, CDCl₃): δ 8.42 (s, 1H), 7.80 (s, 1H), 7.41-7.27 (m, 2H), 3.86-3.81 (m, 1H), 3.41-3.37 (m, 1H), 2.73-2.68 (m, 1H), 2.16-2.04 (m, 2H), 1.75-1.50 (m, 1H) 1.09-1.05 (m, 1H), 0.92-0.90 (m, 1H).

Step K: 3-Bromo-2-(5-fluoropyridin-2-yl)-5,5a,6,6a-tetrahydro-4H-cyclopropa[e]pyrazolo[1,5-a]pyridine. A mixture of 2-(5-fluoropyridin-2-yl)-5,5a,6,6a-tetrahydro-4H-cyclopropa[e]pyrazolo[1,5-a]pyridine (10.0 g, 42.3 mmol, 97% purity, 1.00 eq) and NBS (9.79 g, 55.0 mmol, 1.30 eq) in DMF (100 mL) was stirred at 20° C. for 2 h. The reaction mixture was poured into water (500 mL) and extracted with ethyl acetate (50 mL*3). The organic layer was washed with brine (100 mL), dried over Na₂SO₄, filtered, and concentrated under reduced pressure. The resulting residue was purified (FCC, SiO₂, eluted with petroleum ether/ethyl acetate=10/1) to afford 8.90 g (69%) of the title compound. SFC Purification (Column: Chiralpak AD-3 50×4.6 mm I.D., 3 um Mobile phase: Phase A for CO₂, and Phase B for MeOH (0.05% DEA); Gradient elution: MeOH (0.05% DEA) in CO₂ from 5% to 40% Flow rate: 3 mL/min; Detector: PDA Column Temp: 35 C; Back Pressure: 100 Bar) 3.60 g (40%) of the title compound; MS (ESI): mass calcd. for C₁₃H₁₁BrFN₃ 307.0; m/z found 310.0 [M+H]⁺; ¹H NMR (400 MHz, CDCl₃): δ 8.57-8.55 (m, 1H), 8.00-7.97 (m, 1H), 7.48-7.42 (m, 1H), 3.89-3.84 (m, 1H), 2.88-2.83 (m, 1H), 2.42-2.36 (m, 1H), 2.14-2.12 (m, 1H), 2.12-2.06 (m, 1H), 1.75-1.50 (m, 1H), 1.13-1.07 (m, 1H), 0.88-0.85 (m, 1H); and 4.00 g (45%) of (5a*S,6a*R)-3-bromo-2-(5-fluoropyridin-2-yl)-5, 5a,6,6a-tetrahydro-4H-cyclopropa[e]pyrazolo[1,5-a]pyridine (Intermediate 180).

### Intermediate 180: (5a*S,6a*R)-3-Bromo-2-(5-fluoropyridin-2-yl)-5,5a,6,6a-tetrahydro-4H-cyclopropa[e]pyrazolo[1,5-a]pyridine

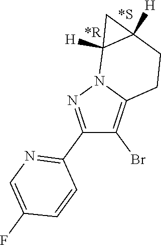 [SMILES: Fc1ccc(-c2nn3c(c2Br)CC[C@H]2C[C@H]23)nc1]

The title compound was isolated via SFC of (Intermediate 179). MS (ESI): mass calcd. for C₁₃H₁₁BrFN₃ 307.0; m/z found 310.0 [M+H]⁺. ¹H NMR (400 MHz, CDCl₃): δ 8.57-8.55 (m, 1H), 8.00-7.97 (m, 1H), 7.48-7.42 (m, 1H), 3.89-3.84 (m, 1H), 2.88-2.83 (m, 1H), 2.42-2.36 (m, 1H), 2.14-2.12 (m, 1H), 2.12-2.06 (m, 1H), 1.75-1.50 (m, 1H), 1.13-1.07 (m, 1H), 0.88-0.85 (m, 1H).

### Intermediate 181: Racemic 3-Bromo-6,6-difluoro-2-(4-fluorophenyl)-5,5a,6,6a-tetrahydro-4H-cyclopropa[e]pyrazolo[1,5-a]pyridine

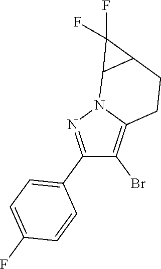 [SMILES: Fc1ccc(-c2nn3c(c2Br)CCC2C3C2(F)F)cc1]

The title compound was prepared in a manner analogous to 3-bromo-2-(5-fluoropyridin-2-yl)-6,7-dihydro-4H-pyrazolo[5,1-c][1,4]oxazine (Intermediate 37, Steps A-B), except using racemic (5aR,6aS)-6,6-difluoro-5,5a,6,6a-tetrahydro-4H-cyclopropa[e][1,2,3]oxadiazolo[3,4-a]pyridin-7-ium-3-olate (Intermediate 14) instead of 6,7-dihydro-4H-[1,2,3]oxadiazolo[4,3-c][1,4]oxazin-8-ium-3-olate (Intermediate 2) and 1-ethynyl-4-fluorobenzene instead of 2-ethynyl-5-fluoropyridine in Step A. MS (ESI): mass calcd. for C₁₃H₉BrF₃N₃ 343.0; m/z found 344.0 [M+H]⁺.

### Intermediate 182: (5a*S,6a*R)-6,6-Difluoro-3-bromo-2-(5-fluoropyridin-2-yl)-5,5a,6,6a-tetrahydro-4H-cyclopropa[e]pyrazolo[1,5-a]pyridine

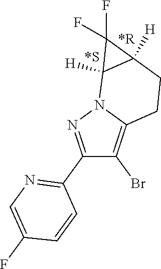 [SMILES: Fc1ccc(-c2nn3c(c2Br)CC[C@H]2[C@@H]3C2(F)F)nc1]

Step A: 2-(tert-Butyl) 3-ethyl 7,7-difluoro-2-azabicyclo[4.1.0]heptane-2,3-dicarboxylate. A mixture of 1-(tert-butyl) 2-ethyl 3,4-dihydropyridine-1,2(2H)-dicarboxylate (Intermediate 179, Step D, 110 g, 422 mmol, 98% purity), NaI (139 g, 928 mmol), and TMSCF₃ (150 g, 1.06 mol) in THF (1.1 L) was heated to 60° C. for 3 hrs under a nitrogen atmosphere. The reaction mixture was then concentrated and the residue purified by silica gel chromatography (eluted with petroleum ether/ethyl acetate=I/O to I/O) to yield the title compound (100 g, 73% yield, 94% purity) as a yellow oil. MS (ESI): mass calcd. for C₁₄H₂₁F₂NO₄ 305.1; m/z found 206.2 [M+H]⁺. ¹H NMR (400 MHz, CDCl₃) δ 4.59-4.70 (m, 1H) 4.14-4.24 (m, 2H) 3.22-3.35 (m, 1H) 1.62-2.00 (m, 5H) 1.43-1.51 (m, 9H) 1.24-1.30 (m, 3H).

Step B. (5a*S,6a*R)-6, 6-Difluoro-3-bromo-2-(5-fluoropyridin-2-yl)-5,5a,6,6a-tetrahydro-4H-cyclopropa[e]pyrazolo[1,5-a]pyridine. The title compound and its enantiomer (Example 183) was made in a manner analogous to (5a*R,6a*S)-3-Bromo-2-(5-fluoropyridin-2-yl)-5,5a,6,6a-tetrahydro-4H-cyclopropa[e]pyrazolo[1,5-a]pyridine (Intermediate 179), Steps F-K, except using 2-(tert-butyl) 3-ethyl 7,7-difluoro-2-azabicyclo[4.1.0]heptane-2,3-dicarboxylate instead of 2-(tert-Butyl) 3-ethyl 2-azabicyclo[4.1.0]heptane-2,3-dicarboxylate (Intermediate 179, Step E) in Step F, and using SFC (column: DAICEL CHIRALCEL OD (250 mm*50 mm, 10 um); mobile phase: [0.1% NH₃H₂O EtOH]) instead of SFC (Column: Chiralpak AD-3 50×4.6 mm I.D., 3 um Mobile phase: Phase A for CO₂, and Phase B for MeOH (0.05% DEA); Gradient elution: MeOH (0.05% DEA) in CO₂ from 5% to 40% Flow rate: 3 mL/min; Detector: PDA Column Temp: 35 C; Back Pressure: 100 Bar) in Step K. MS (ESI): mass calcd. for C₁₃H₉BrF₃N₃ 343.0; m/z found 344.0 [M+H]⁺. ¹H NMR (400 MHz, CDCl₃): δ 8.59 (br d, J=2.32 Hz, 1H) 8.02 (dd, J=8.80, 4.40 Hz, 1H) 7.48 (td, J=8.44, 2.93 Hz, 1H) 4.29 (dd, J=10.88, 6.24 Hz, 1H) 2.81-2.92 (m, 1H) 2.69-2.80 (m, 1H) 2.42 (ddtd, J=14.15, 11.32, 5.60, 5.60, 2.81 Hz, 1H) 2.12-2.24 (m, 2H); and (5a*R,6a*S)-6,6-Difluoro-3-bromo-2-(5-fluoropyridin-2-yl)-5,5a,6,6a-tetrahydro-4H-cyclopropa[e]pyrazolo[1,5-a]pyridine (Intermediate 183).

### Intermediate 183: (5a*R,6a*S)-6,6-Difluoro-3-bromo-2-(5-fluoropyridin-2-yl)-5,5a,6,6a-tetrahydro-4H-cyclopropa[e]pyrazolo[1,5-a]pyridine

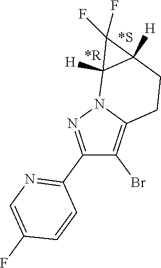 [SMILES: Fc1ccc(-c2nn3c(c2Br)CC[C@@H]2[C@H]3C2(F)F)nc1]

The title compound was isolated via SFC in Intermediate 182. MS (ESI): mass calcd. for C₁₃H₉BrF₃N₃ 343.0; m/z found 344.0 [M+H]⁺. ¹H NMR (400 MHz, CDCl₃): δ 8.50-8.66 (m, 1H) 7.93-8.08 (m, 1H) 7.43-7.52 (m, 1H) 4.16-4.38 (m, 1H) 2.80-2.92 (m, 1H) 2.67-2.79 (m, 1H) 2.35-2.47 (m, 1H) 2.14-2.22 (m, 2H).

### Intermediate 184: 3-Bromo-2-(5-fluoropyridin-2-yl)-6,6-bis(methyl-d₃)-6,7-dihydro-4H-pyrazolo[5,1-c][1,4]oxazine-4,4,7,7-d₄

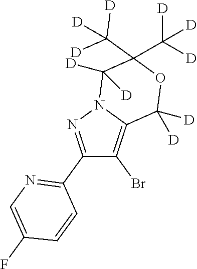

Step A: 2-(Benzyloxy)-2,2-di-D-acetic acid. Na (62 mg, 2.7 mmol) was added to CD₃OD (10 mL). The reaction mixture was stirred at r.t. for 1 h, methyl 2-(benzyloxy)acetate (5 g, 27.7 mmol) was added to the above solution. The reaction mixture was stirred at 70° C. for 48 h before being cooled to r.t. The solvent was removed under reduced pressure and another 10 mL of CD₃OD was added. The reaction mixture was stirred at 70° C. for another 16 h before being cooled to r.t. The reaction mixture was poured it into sat. NH₄Cl (50 mL) at 0° C. and extracted with EtOAc (10 mL×5). The combined organic extracts were dried over anhydrous Na₂SO₄, filtered, and concentrated to dryness under reduced pressure to afford the title compound (4.5 g, 97%). ¹H NMR (400 MHz, CDCl₃) δ7.46-7.29 (m, 5H), 4.64 (s, 2H).

Step B: Methyl 2-(benzyloxy)-2,2-di-D-acetate. To a solution of 2-(benzyloxy)-2,2-di-D-chloroacetic acid (4.8 g, crude) in MeOH (20 mL) was added SOCl₂ (2.1 mL, 28 mmol) dropwise at 0° C. The resultant mixture was stirred for 16 h then gradually warmed to room temperature. The reaction mixture was then poured into sat. NH₄Cl sol'n (50 mL) and extracted with EtOAc (10 mL×5). The combined organic extracts were dried over anhydrous Na₂SO₄, filtered, and concentrated to dryness under reduced pressure to afford the title compound (2.8 g, crude, 53% yield, ˜98% purity). ¹H NMR (400 MHz, CDCl₃) δ7.40-7.25 (m, 5H), 4.64 (s, 2H), 3.78 (s, 3H).

Step C: 1-(Benzyloxy)-2-(methyl-d₃)propan-1,1,3,3,3-d₅-2-ol. To a cooled (0° C.) solution of methyl 2-(benzyloxy)-2,2-di-D-acetate (500 mg, 2.7 mmol) in THF (20 mL) was added methyl-d₃ magnesium iodide (11 mL, 11 mmol, 1 M in ethoxyethane) drop wise. The reaction mixture was stirred for 16 hrs. The reaction mixture was gradually warmed to r.t. The reaction mixture was quenched with sat. NH₄Cl (30 mL) and extracted with EtOAc (60 mL×3). The combined organic extracts were dried over anhydrous Na₂SO₄, filtered, and concentrated to dryness under reduced pressure. The resulting residue was purified by FCC (SiO₂, eluent: pet ether:EtOAc=20:1 to 3:1) to afford the title compound (300 mg, crude, 59%). ¹H NMR (400 MHz, CDCl₃): δ 7.44-7.28 (m, 5H), 4.64-4.54 (m, 2H), 4.59 (s, 1H), 2.39 (s, 1H).

Step D: 2-(Methyl-d₃)propane-1,1,3,3,3-d₅-1,2-diol. 1-(Benzyloxy)-2-(methyl-d₃)propan-1,1,3,3,3-d₅-2-ol (1.5 g, 8.0 mmol), EA (50 mL) and dry Pd/C (0.8 g) were added to a 100 mL hydrogenation bottle. The resultant mixture was stirred under H₂ (50 psi) at 50° C. for 5 h. The reaction mixture was cooled to r.t. and the resulting suspension was filtered through a pad of Celite®. The pad washed with EA (200 mL) and the filtrate was concentrated to dryness under reduced pressure to afford the title product (750 mg, 95%) as a yellow oil, which was used in the next step without further purification.

Step E: 2-Hydroxy-2-(methyl-d₃)propyl-1,1,3,3,3-d₅ 4-methylbenzenesulfonate. To a solution 2-(methyl-d₃)propane-1,1,3,3,3-d₅-1,2-diol (700 mg, 7.13 mmol), DMAP (174 mg, 1.42 mmol), and TEA (4.96 ml, 35.7 mmol) in dichloromethane (20 mL) was added TosCl (1.63 g, 8.55 mmol). The resultant mixture was stirred at r.t. for 12 h. The reaction mixture was concentrated to dryness under reduced pressure. The resulting residue was purified FCC (SiO₂, pet ether:EtOAc=20:1 to 3:1) to afford the compound (1.0 g, 56%) as a yellow oil. ¹H NMR (400 MHz, CDCl₃): δ 7.80 (d, J=8.3 Hz, 2H), 7.36 (d, J=8.0 Hz, 2H), 2.45 (s, 3H).

Step F: 2-(5-(((tert-Butyldimethylsilyl)oxy)methyl-d₂)-1H-pyrazol-3-yl)-5-fluoropyridine. To a solution of (3-(5-fluoropyridin-2-yl)-1D-pyrazol-5-yl)methanol (Intermediate 36, 3.0 g, 15 mmol), and 1H-imidazole (3.14 g, 46.1 mmol), in dichloromethane (30 mL), in DMF (3 mL) was added TBSCl (3.48 g, 23.1 mmol). The reaction mixture was stirred at r.t. for 30 min. The reaction mixture was filtered through a pad of Celite® and the pad was washed with EtOAc (200 mL). The combined organics were concentrated to dryness under reduced pressure. The resulting residue was purified FCC (SiO₂, (eluent: pet ether:EtOAc=1:0 to 3:1) to afford the title compound (4.0 g, 78%), as a white solid. LCMS (ESI): mass calcd. for C₁₅H₂₀D₂FN₃OSi 309.2 m/z, found 310.2 [M+1]⁺. ¹H NMR (400 MHz, CDCl₃): δ 8.47 (d, J=2.8 Hz, 1H), 7.92-7.69 (m, 1H), 7.58-7.33 (m, 1H), 6.70 (s, 1H), 0.98-0.93 (m, 1H), 0.95 (s, 7H), 1.01-0.92 (m, 1H), 0.13 (s, 6H).

Step G: 1-(3-(5-Fluoropyridin-2-yl)-5-(hydroxymethyl-d₂)-1H-pyrazol-1-yl)-2-(methyl-d₃)propan-1,1,3,3,3-d₅-2-ol. A solution of 2-hydroxy-2-(methyl-d₃)propyl-1,1,3,3,3-d₅ 4-methylbenzenesulfonate (800 mg, 3.17 mmol), 2-(5-(((tert-butyldimethylsilyl)oxy)methyl-d₂)-1H-pyrazol-3-yl)-5-fluoropyridine (981 mg, 3.17 mmol), Cs₂CO₃ (5.2 g, 16 mmol), and KI (526 mg, 3.17 mmol), in DMA (10 mL) was heated at 120° C. via microwave irradiation for 0.5 h. The reaction mixture was cooled to r.t. and poured into water (50 mL). The reaction mixture was extracted with EtOAc (80 mL×3) and the combined organic layers were washed with brine (50 mL), dried over anhydrous Na₂SO₄, filtered and concentrated under reduced pressure. The resulting residue was purified FCC (SiO₂, pet ether:EtOAc=10:1 to 0:1) to afford the compound (400 mg, crude, 46%) as a clean oil.

Step H: 2-(5-Fluoropyridin-2-yl)-6,6-bis(methyl-d₃)-6,7-dihydro-4H-pyrazolo[5,1-c][1,4]oxazine-4,4,7,7-d₄. KOH (328 mg, 5.85 mmol) and H₂O (4 mL) were added to a solution of 1-(3-(5-fluoropyridin-2-yl)-5-(hydroxymethyl-d₂)-1H-pyrazol-1-yl)-2-(methyl-d₃)propan-1,1,3,3,3-d₅-2-ol (400 mg, 1.46 mmol), and TsCl (418 mg, 2.19 mmol) in dioxane (8 mL). The resultant mixture was stirred at 100° C. for 12 h. The reaction mixture was cooled and combined with an additional batch of the same reaction. The combined reaction mixtures were quenched with water (30 mL) and extracted with EtOAc (50 mL×3). The combined organic extracts were dried over anhydrous Na₂SO₄, filtered and concentrated to dryness under reduced pressure. The resulting residue was purified FCC (SiO₂, eluent: pet ether:EtOAc=1:0 to 1:1) to afford the title compound as white oil. LCMS (ESI): mass calcd. for C₁₃H₄D₁₀FN₃O 257.2; m/z found 258.2 [M+H]⁺.

Step I: 3-Bromo-2-(5-fluoropyridin-2-yl)-6,6-bis(methyl-d₃)-6,7-dihydro-4H-pyrazolo[5,1-c][1,4]oxazine-4,4,7,7-d₄. NBS (91 mg, 0.51 mmol) was added to a 0° C. (ice/water) solution of 2-(5-fluoropyridin-2-yl)-6,6-bis(methyl-d₃)-6,7-dihydro-4H-pyrazolo[5,1-c][1,4]oxazine-4,4,7,7-d₄ (110 mg, 0.43 mmol) in dichloromethane (10 mL). The reaction mixture was stirred for 0.5 h, and warmed to r.t. The reaction mixture was stirred at r.t. for 2 h. H₂O (30 mL) was added to the reaction mixture and extracted with dichloromethane (50 mL×3) The combined organic extracts were washed with brine (10 mL), dried over anhydrous Na₂SO₄, filtered, and concentrated to dryness under reduced pressure. The resulting residue was purified FCC (SiO₂, eluent: pet ether:EtOAc=1:0 to 3:1) to afford the crude title compound (0.19 g) as brown oil. LCMS (ESI): mass calcd. for C₁₃H₃BrD₁₀FN₃O 335.09; m/z found 336.1 [M+H]⁺.

### Intermediate 185: 3-Bromo-2-(4-fluorophenyl)-6-methyl-6-(trifluoromethyl)-6,7-dihydro-4H-pyrazolo[5,1-c][1,4]oxazine

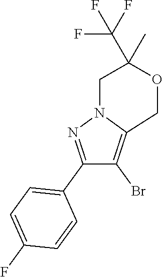 [SMILES: CC1(C(F)(F)F)Cn2nc(-c3ccc(F)cc3)c(Br)c2CO1]

The title compound was prepared in a manner analogous to Intermediate 115, Steps A-C, except using ethyl 3-(4-fluorophenyl)-1H-pyrazole-5-carboxylate (Intermediate 33) instead of ethyl 3-(5-fluoropyridin-2-yl)-1H-pyrazole-5-carboxylate (Intermediate 34) in Step A. MS (ESI): mass calcd. for C₁₄H₁₁BrF₄N₂O, 378.0; m/z found 379.1 [M+H]⁺.

### Intermediate 186: 3-Bromo-2-(5-fluoropyridin-2-yl)-6-methyl-6-(trifluoromethyl)-6,7-dihydro-4H-pyrazolo[5,1-c][1,4]oxazine

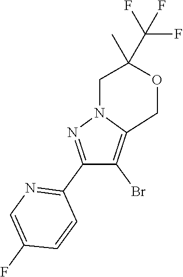 [SMILES: CC1(C(F)(F)F)Cn2nc(-c3ccc(F)cn3)c(Br)c2CO1]

Step A. 1-(5-(((tert-Butyldimethylsilyl)oxy)methyl)-3-(5-fluoropyridin-2-yl)-1H-pyrazol-1-yl)propan-2-one. 1-Bromopropan-2-one (2.02 g, 14.7 mmol) was added to a mixture consisting of 2-(5-(((tert-Butyldimethylsilyl)oxy)methyl)-1H-pyrazol-3-yl)-5-fluoropyridine (Intermediate 35, 2.0 g, 6.5 mmol), and Cs₂CO₃ (6.4 g, 20 mmol), in MeCN (80 mL). The reaction mixture was stirred at room-temperature for 16 hours. The reaction mixture was quenched with H₂O (150 mL) and extracted with ethyl acetate (60 mL×3). The combined organic extracts were dried over anhydrous Na₂SO₄, filtered and concentrated to dryness. The resulting residue was purified by FCC (SiO₂, eluent: ethyl acetate:petroleum ether=1:3) to afford the title compound (1.5 g, 63%) as yellow oil. MS (ESI): mass calcd. for C₁₂H₁₈BrN₃OSi 363.2; m/z found 364.4 [M+H]⁺.

Step B. 1,1,1-Trifluoro-3-(3-(5-fluoropyridin-2-yl)-5-(hydroxymethyl)-1H-pyrazol-1-yl)-2-methylpropan-2-ol. TMSCF₃ (2.45 g, 17.2 mmol) was added dropwise to a 0° C. (ice/water) solution of 1-(5-(((tert-butyldimethylsilyl)oxy)methyl)-3-(5-fluoropyridin-2-yl)-1H-pyrazol-1-yl)propan-2-one (2.5 g, 6.9 mmol) in THF (80 mL). TBAF (5.2 mL, 1 M in THF, 5.2 mmol) was added dropwise at 0° C. to the reaction mixture. The reaction mixture was stirred at room-temperature for 16 hours. The reaction mixture was cooled to 0° C., quenched with sat. aq. H₂O (50 mL) and extracted with ethyl acetate (50 mL×3). The combined organic extracts were washed with brine (50 mL), dried over anhydrous Na₂SO₄, and concentrated under reduced pressure to dryness. The resulting residue was purified by FCC (SiO₂, eluent: petroleum ether:ethyl acetate=1:0 to 1:1) to afford the title compound (1.4 g, 64%) as light yellow oil. MS (ESI): mass calcd. for C₁₃H₁₃F₄N₃O₂ 319.1 m/z found 319.9 [M+H]⁺.

Step C. 2-(5-Fluoropyridin-2-yl)-6-methyl-6-(trifluoromethyl)-6,7-dihydro-4H-pyrazolo[5,1-c][1,4]oxazine. KOH (743 mg, 13.3 mmol) and H₂O (10 mL) were added to a solution of 1,1,1-trifluoro-3-(3-(5-fluoropyridin-2-yl)-5-(hydroxymethyl)-1H-pyrazol-1-yl)-2-methylpropan-2-ol (1.4 g, 4.4 mmol), and TsCl (1.6 g, 8.4 mmol), in 1,4-dioxane (50 mL). The resultant mixture was stirred at 100° C. for 16 hours. The reaction mixture was cooled to room temperature, quenched with water (100 mL) and extracted with ethyl acetate (50 mL×3). The combined organic extracts were dried over anhydrous Na₂SO₄, filtered, and concentrated to dryness under reduced pressure. The resulting residue was purified by FCC (SiO₂, eluent: petroleum ether:ethyl acetate=20:1 to 3:1) to afford the title compound (1.0 g, 74%) as a white solid. MS (ESI): mass calcd. for C₁₃H₁₁F₄N₃O 301.2 m/z found 301.9 [M+H]⁺.

Step D. 3-Bromo-2-(5-fluoropyridin-2-yl)-6-methyl-6-(trifluoromethyl)-6,7-dihydro-4H-pyrazolo[5,1-c][1,4]oxazine. NBS (650 mg, 3.65 mmol) was added to a solution consisting of 2-(5-fluoropyridin-2-yl)-6-methyl-6-(trifluoromethyl)-6,7-dihydro-4H-pyrazolo[5,1-c][1,4]oxazine (1.0 g, 3.32 mmol) in dichloromethane (20 mL). The resultant mixture was stirred at room temperature for 20 minutes. The reaction mixture was poured it into sat. NaHCO₃(10 mL) and extracted with dichloromethane (10 mL×2). The combined organic extracts were washed with brine (10 mL), dried over anhydrous Na₂SO₄, filtered, and concentrated to dryness under reduced pressure. The resulting residue was purified by FCC (SiO₂, eluent: petroleum ether:ethyl acetate=10:1 to 5:1) to afford the product (1.2 g, 95%) as yellow solid. MS (ESI): mass calcd. for C₁₃H₁₀BrF₄N₃O 379.0 m/z found 381.7 [M+H]⁺.

### Intermediate 187: (*R)-3-Bromo-2-(5-fluoropyridin-2-yl)-6-methyl-6-(trifluoromethyl)-6,7-dihydro-4H-pyrazolo[5,1-c][1,4]oxazine

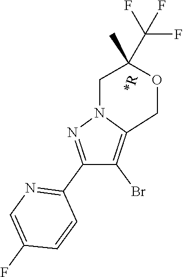 [SMILES: C[C@]1(C(F)(F)F)Cn2nc(-c3ccc(F)cn3)c(Br)c2CO1]

The title compound was prepared by chiral SFC purification (Stationary phase: Reflect I Cellulose B Sum 250×21 mm, Mobile phase: 20% methanol, 80% CO₂; Flow rate 42 mL/min, monitor at 220 nm) of 3-bromo-2-(5-fluoropyridin-2-yl)-6-methyl-6-(trifluoromethyl)-6,7-dihydro-4H-pyrazolo[5,1-c][1,4]oxazine (Intermediate 186). MS (ESI): mass calcd. for C₁₃H₁₀BrF₄N₃O 379.0 m/z found 380.0 [M+H]⁺.

### Intermediate 188: (*S)-3-Bromo-2-(5-fluoropyridin-2-yl)-6-methyl-6-(trifluoromethyl)-6,7-dihydro-4H-pyrazolo[5,1-c][1,4]oxazine

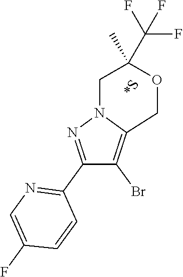 [SMILES: C[C@@]1(C(F)(F)F)Cn2nc(-c3ccc(F)cn3)c(Br)c2CO1]

The title compound was prepared by chiral SFC purification (Stationary phase: Reflect I Cellulose B 5 um 250×21 mm, Mobile phase: 20% methanol, 80% CO₂; Flow rate 42 mL/min, monitor at 220 nm) of 3-bromo-2-(5-fluoropyridin-2-yl)-6-methyl-6-(trifluoromethyl)-6,7-dihydro-4H-pyrazolo[5,1-c][1,4]oxazine (Intermediate 186). MS (ESI): mass calcd. for C₁₃H₁₀BrF₄N₃O 379.0 m/z found 380.0 [M+H]⁺.

### Intermediate 189: 3-Bromo-2-(5-fluoropyridin-2-yl)-6-(methyl-d₃)-6-(trifluoromethyl)-6,7-dihydro-4H-pyrazolo[5,1-c][1,4]oxazine

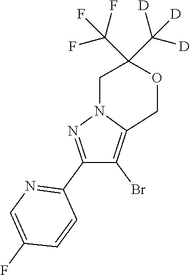 [SMILES: [2H]C([2H])([2H])C1(C(F)(F)F)Cn2nc(-c3ccc(F)cn3)c(Br)c2CO1]

Step A: 1-(Benzyloxy)propan-3,3,3-d₃-2-ol. methyl-d₃-magnesium-iodide (67 mL, 67 mmol) was added dropwise to a 0° C. (ice/water) solution consisting of 2-(benzyloxy)acetaldehyde (5.0 g, 33 mmol) in THF (60 mL). The reaction mixture was stirred for 1 hour. The reaction mixture was gradually warmed to room-temperature and then stirred at room-temperature for 4 hours. The reaction mixture was quenched with sat. NH₄Cl (50 mL) and extracted with ethyl acetate (50 mL×3). The combined organic extracts were washed with brine (30 mL×2), dried over anhydrous Na₂SO₄, filtered, and concentrated to dryness under reduced pressure. The resulting residue was purified by FCC (SiO₂, eluent: petroleum ether:ethyl acetate=1:0 to 4:1) to afford the title compound (3.8 g, 66%) as a yellow oil. ¹H NMR (400 MHz, DMSO-d₆): δ 7.36-7.31 (m, 4H), 7.31-7.26 (m, 1H), 4.60 (d, J=4.5 Hz, 1H), 4.49 (s, 2H), 3.76 (q, J=5.4 Hz, 1H), 3.34-3.30 (m, 1H), 3.26-3.20 (m, 1H).

Step B: 1-(Benzyloxy)propan-2-one-3,3,3-d₃. A solution of 1-(benzyloxy)propan-3,3,3-d₃-2-ol (3.8 g, 22 mmol) in dichloromethane (50 mL) was stirred at room-temperature for 5 mins. Dess-Martin (11.5 g, 27.1 mmol) was added to the reaction mixture, then the resulting mixture was stirred at room-temperature for 16 hours. The reaction mixture was concentrated to dryness under reduced pressure. The resulting residue was purified by FCC (SiO₂, eluent: petroleum ether:ethyl acetate=1:0 to 3:1) to afford the title compound (3.4 g, 86%) as a colorless oil. ¹H NMR (400 MHz, DMSO-d₆) δ 7.37-7.33 (m, 4H), 7.32-7.27 (m, 1H), 4.51 (s, 2H), 4.15 (s, 2H).

Step C: 2-((Benzyloxy)methyl)-1,1,1-trifluoropropan-3,3,3-d₃-2-ol. TMSCF₃ (5.0 mL, 34 mmol) was added to a solution consisting of 1-(benzyloxy)propan-2-one-3,3,3-d₃. (3.3 g, 20 mmol) in 1,2-dimethoxyethane (50 mL). The reaction mixture was stirred at room-temperature for 5 min and then TBAF (4 mL, 4 mmol) was added dropwise to the above mixture. The reaction mixture was stirred at room-temperature for 16 hours. The reaction mixture was diluted with H₂O (30 mL) and extracted with dichloromethane (20 ml×2). The combined organic extracts were concentrated to dryness under reduced pressure. The resulting residue was purified by FCC (SiO₂, eluent: petroleum ether:ethyl acetate=1:0 to 5:1) to afford the title compound (2.7 g, 55%) as a yellow oil. ¹H NMR (400 MHz, DMSO-d₆) δ 7.39-7.27 (m, 5H), 6.03 (s, 1H), 4.58-4.51 (m, 2H), 3.55-3.49 (m, 1H), 3.45-3.40 (m, 1H).

Step D: 3,3,3-Trifluoro-2-(methyl-d₃)propane-1,2-diol. A solution of 2-((benzyloxy)methyl)-1,1,1-trifluoropropan-3,3,3-d₃-2-ol (2.6 g, 11 mmol), dry Pd/C (1 g, 10 wt. %), and ethyl acetate (30 mL) was stirred under H₂ (50 psi) at 50° C. for 36 hours. The suspension was filtered through a pad of Celite® and the pad washed with ethyl acetate (10 mL×3). The filtrate was concentrated to dryness under reduced pressure to afford the title compound (1.4 g) as a colorless oil, which was used in the next step without further purification. ¹H NMR (400 MHz, CDCl₃): δ 3.91 (d, J=11.9 Hz, 1H), 3.57-3.50 (m, 1H), 3.31 (s, 1H), 2.21 (s, 1H).

Step E: 3,3,3-Trifluoro-2-hydroxy-2-(methyl-d₃)propyl 4-methylbenzenesulfonate. TsCl (2.43 g, 12.7 mmol) was added to a solution consisting of 3,3,3-trifluoro-2-(methyl-d₃)propane-1,2-diol (1.25 g, crude), TEA (2.9 mL, 21 mmol), and DMAP (105 mg, 0.859 mmol), in dichloromethane (15 mL). The resultant mixture was stirred at room-temperature for 16 hours. The reaction mixture concentrated to dryness under reduced pressure to afford the title compound (1.75 g), which was used in the next step without further purification.

Step F: 2-(5-Fluoropyridin-2-yl)-6-(methyl-d₃)-6-(trifluoromethyl)-6,7-dihydro-4H-pyrazolo[5,1-c][1,4]oxazine. To a solution consisting of 2-(5-(((tert-Butyldimethylsilyl)oxy)methyl)-1H-pyrazol-3-yl)-5-fluoropyridine (Intermediate 35, 1.5 g, 4.9 mmol), Cs₂CO₃ (4.75 g, 14.6 mmol), and KI (805 mg, 4.85 mmol) in DMA (20 mL) was added 3,3,3-trifluoro-2-hydroxy-2-(methyl-d₃)propyl 4-methylbenzenesulfonate (1.75 g, 5.81 mmol). The resultant mixture was stirred at 120° C. for 0.5 hours. The reaction mixture was poured into water (20 mL) and extracted with dichloromethane (15 mL×2). The combined organic extracts were washed with brine (10 mL×2), dried over anhydrous Na₂SO₄, filtered, and concentrated to dryness under reduced pressure. The resulting residue was purified by FCC (SiO₂, eluent: petroleum ether:ethyl acetate=1:0 to 4:1) to afford the title compound (320 mg) as a yellow solid. MS (ESI): mass calcd. for C₁₃H₈D₃F₄N₃O 304.10; m/z found 304.9 [M+H]⁺. ¹H NMR (400 MHz, CDCl₃): δ 8.47 (d, J=2.9 Hz, 1H), 7.90 (dd, J=4.5, 8.8 Hz, 1H), 7.47-7.41 (m, 1H), 6.64-6.62 (m, 1H), 5.11-5.04 (m, 1H), 5.00-4.92 (m, 1H), 4.45-4.38 (m, 1H), 4.21-4.15 (m, 1H).

Step G: 3-Bromo-2-(5-fluoropyridin-2-yl)-6-(methyl-d₃)-6-(trifluoromethyl)-6,7-dihydro-4H-pyrazolo[5,1-c][1,4]oxazine. NBS (200 mg, 1.12 mmol) was added to a solution consisting of 2-(5-fluoropyridin-2-yl)-6-(methyl-d₃)-6-(trifluoromethyl)-6,7-dihydro-4H-pyrazolo[5,1-c][1,4]oxazine (280 mg, 0.920 mmol) in dichloromethane (5 mL). The reaction mixture was stirred at room-temperature for 1 hour. The reaction mixture was quenched with H₂O (6 mL) and extracted with dichloromethane (5 mL×2). The combined organic extracts were dried over anhydrous Na₂SO₄, filtered, and concentrated to dryness under reduced pressure. The resulting residue was purified by FCC (SiO₂, eluent: petroleum ether:ethyl acetate=1:0 to 4:1) to afford the title compound (350 mg) as a yellow solid. MS (ESI): mass calcd. for C₁₃H₇D₃BrF₄N₃O 382.01; m/z found 382.9 [M+H]⁺. ¹H NMR (400 MHz, CDCl₃): δ 8.59 (d, J=3.0 Hz, 1H), 8.00 (dd, J=4.4, 8.8 Hz, 1H), 7.52-7.46 (m, 1H), 5.03-4.97 (m, 1H), 4.90-4.84 (m, 1H), 4.44-4.39 (m, 1H), 4.19-4.15 (m, 1H).

### Intermediate 190: 3′-Bromo-2′-(5-fluoropyridin-2-yl)-4′,7′-dihydrospiro[cyclopropane-1,6′-pyrazolo[5,1-c][1,4]oxazine]

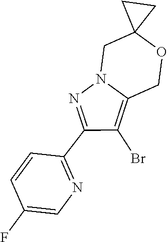 [SMILES: Fc1ccc(-c2nn3c(c2Br)COC2(CC2)C3)nc1]

Step A. Methyl 1-((tert-butyldimethylsilyl)oxy)cyclopropanecarboxylate. TBSCl (9.74 g, 64.6 mmol) was added to a solution consisting of methyl 1-hydroxycyclopropanecarboxylate (5.0 g, 43 mmol), and 1H-imidazole (8.80 g, 129 mmol), in dichloromethane (100 mL). The reaction mixture was stirred at room-temperature for 1 hour. The reaction mixture was filtered through a pad of Celite® and the pad was washed with ethyl acetate (20 mL×3). The combined organic extracts were concentrated to dryness under reduced pressure. The resulting residue was purified by FCC (SiO₂, eluent: petroleum ether:ethyl acetate=1:0 to 20:1) to afford the title compound (5.7 g, 56%) as a colorless oil. ¹H NMR (400 MHz, CDCl₃): δ 3.72 (s, 3H), 1.34-1.30 (m, 2H), 1.10-1.05 (m, 2H), 0.87 (s, 9H), 0.15 (s, 6H).

Step B. (1-((tert-Butyldimethylsilyl)oxy)cyclopropyl)methanol. DIBAL-H (50 mL, 75 mmol) was added dropwise to a 0° C. (ice/water) solution consisting of methyl 1-((tert-butyldimethylsilyl)oxy)cyclopropanecarboxylate (5.7 g, 25 mmol) in THF (100 mL) under N₂. The reaction mixture was stirred at 0° C. (ice/water) for 2 hours. Na₂SO₄·10 H₂O (30 g) was added in portions to the mixture at 0° C. (ice/water), and then stirred for 1 hour. The suspension was filtered through a pad of Celite® and the pad washed with ethyl acetate (50 mL×3). The filtrate was concentrated to dryness under reduced pressure. The resulting residue was purified by FCC (SiO₂, eluent: petroleum ether:ethyl acetate=1:0 to 10:1) to afford the title compound (2.8 g, 55%) as colorless oil. ¹H NMR (400 MHz, CDCl₃): δ 3.56 (d, J=5.8 Hz, 2H), 0.87 (s, 9H), 0.80-0.76 (m, 2H), 0.61-0.57 (m, 2H), 0.13 (s, 6H).

Step C. Ethyl 1-((1-((tert-butyldimethylsilyl)oxy)cyclopropyl)methyl)-3-(5-fluoropyridin-2-yl)-1H-pyrazole-5-carboxylate. DIAD (4.5 mL, 23 mmol) was added dropwise to a 0° C. (ice/water) solution consisting of (1-((tert-butyldimethylsilyl)oxy)cyclopropyl)methanol (2.3 g, 11 mmol), ethyl 3-(5-fluoropyridin-2-yl)-1H-pyrazole-5-carboxylate (2.68 g, 11.4 mmol), and PPh₃ (6.0 g, 23 mmol) in THF (50 mL) under N₂. The resultant mixture was stirred for 0.5 hours. The reaction mixture was gradually warmed to room-temperature and then stirred at this temperature for 16 hours. The reaction mixture was quenched with H₂O (50 mL) and extracted with dichloromethane (30 mL×2). The combined organic extracts were dried over anhydrous Na₂SO₄, filtered, and concentrated to dryness under reduced pressure. The resulting residue was purified by FCC (SiO₂, eluent: petroleum ether:ethyl acetate=1:0 to 20:1) to afford the title compound (2.6 g, 48%) as a yellow oil. MS (ESI): mass calcd. for C₂₁H₃₀FN₃O₃Si 419.2; m/z found 420.6 [M+H]⁺.

Step D. (1-((1-((tert-Butyldimethylsilylloxy)cyclopropyl)methyl)-3-(5-fluoropyridin-2-yl)-1H-pyrazol-5-yl)methanol. LiAlH₄ (520 mg, 13.7 mmol) was added in portions to a 0° C. (ice/water) solution of ethyl 1-((1-((tert-butyldimethylsilyl)oxy)cyclopropyl)methyl)-3-(5-fluoropyridin-2-yl)-1H-pyrazole-5-carboxylate (2.6 g, 5.5 mmol) in THF (50 mL) under N₂. The resultant mixture was stirred for 2 hours at 0° C. (ice/water). H₂O (0.6 mL) was added dropwise to the reaction mixture at 0° C. (ice/water), and then 15% aq. NaOH (0.6 mL) was added to the mixture. Then MgSO₄ (3 g) was added. The reaction mixture was stirred 0° C. for 10 min. The resultant was filtered through a pad of Celite® and the pad washed with MeOH (10 mL×3). The filtrate was concentrated to dryness under reduced pressure to give the title compound (2 g) as a yellow oil, which was used in the next step without further purification. MS (ESI): mass calcd. for C₁₉H₂₈FN₃O₂Si 377.2 m/z found 378.0 [M+H]⁺.

Step E. 1-((3-(5-Fluoropyridin-2-yl)-5-(hydroxymethyl)-1H-pyrazol-1-yl)methyl)cyclopropanol. TBAF (10.6 mL, 1 M in THF, 10.6 mmol) was added to a solution of (1-((1-((tert-butyldimethylsilyl)oxy)cyclopropyl)methyl)-3-(5-fluoropyridin-2-yl)-1H-pyrazol-5-yl)methanol (2 g) in THF (30 mL). The resultant solution was stirred for 2 hours at room-temperature. The reaction mixture was quenched with sat. NH₄Cl (30 mL) and extracted with ethyl acetate (20 mL×2). The combined organic extracts were dried over anhydrous Na₂SO₄, filtered, and concentrated to dryness under reduced pressure. The resulting residue was purified by FCC (SiO₂, eluent: petroleum ether:ethyl acetate=20:1 to 1:2) to afford the title compound (1 g) as white solid. MS (ESI): mass calcd. for C₁₃H₁₄FN₃O₂ 263.1; m/z found 263.9 [M+H]⁺.

Step F. 2′-(5-Fluoropyridin-2-yl)-4′,7′-dihydrospiro[cyclopropane-1,6′-pyrazolo[5,1-c][1,4]oxazine]. A solution of KOH (1.28 g, 22.8 mmol) in H₂O (5 mL) was added to a solution of 1-((3-(5-fluoropyridin-2-yl)-5-(hydroxymethyl)-1H-pyrazol-1-yl)methyl)cyclopropanol (1.0 g, 3.8 mmol), and TsCl (1.1 g, 5.8 mmol), in 1,4-dioxane (10 mL). The resultant mixture was stirred at 100° C. for 16 hours. The reaction mixture was quenched with H₂O (20 mL) and extracted with dichloromethane (15 mL×2). The combined organic extracts were dried over anhydrous Na₂SO₄, filtered, and concentrated to dryness under reduced pressure. The resulting residue was purified by FCC (SiO₂, eluent: petroleum ether:ethyl acetate=1:0 to 2:1) to afford the title compound (650 mg, 65%) as a white solid. MS (ESI): mass calcd. for C₁₃H₁₂FN₃O 245.1; m/z found 246.2 [M+H]⁺. ¹H NMR (400 MHz, DMSO-d₆) δ 8.56 (d, J=2.9 Hz, 1H), 7.95 (dd, J=4.6, 8.8 Hz, 1H), 7.78-7.72 (m, 1H), 6.63 (s, 1H), 4.82 (s, 2H), 4.21 (s, 2H), 1.03-0.98 (m, 2H), 0.81-0.77 (m, 2H).

Step G. 3′-Bromo-2′-(5-fluoropyridin-2-yl)-4′,7′-dihydrospiro[cyclopropane-1,6′-pyrazolo[5,1-c][1,4]oxazine]. NBS (525 mg, 2.95 mmol) was added to a solution of 2′-(5-fluoropyridin-2-yl)-4′,7′-dihydrospiro[cyclopropane-1,6′-pyrazolo[5,1-c][1,4]oxazine] (600 mg, 2.45 mmol) in dichloromethane (10 mL). Then the reaction mixture was stirred at room-temperature for 1 hour. The reaction mixture was quenched with H₂O (15 mL) and extracted with dichloromethane (10 mL×2). The combined organic extracts were dried over anhydrous Na₂SO₄, filtered, and concentrated to dryness under reduced pressure. The resulting residue was purified by FCC (SiO₂, eluent: petroleum ether:ethyl acetate=1:0 to 3:1) to afford the title compound (800 mg, 98%) as a yellow solid. MS (ESI): mass calcd. for C₁₃H₁₁BrFN₃O 323.0; m/z found 323.9 [M+H]⁺.

### Intermediate 191: 3′-Bromo-2,2-difluoro-2′-(5-fluoropyridin-2-yl)-4′H,7′H-spiro[cyclopropane-1,6′-pyrazolo[5,1-c][1,4]oxazine]

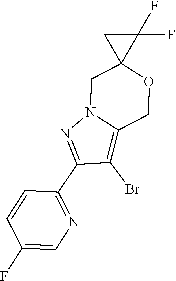 [SMILES: Fc1ccc(-c2nn3c(c2Br)COC2(C3)CC2(F)F)nc1]

Step A. Methyl 2-(benzyl(prop-2-yn-1-yl)amino)-3-hydroxypropanoate. K₂CO₃ (6.6 g, 47.8 mmol) and KI (1.6 g, 9.64 mmol) were added to a solution of methyl 2-(benzylamino)-3-hydroxypropanoate (10 g, 47.8 mmol), and 3-bromoprop-1-yne (8.5 g, 57.2 mmol), in MeCN (150 mL). The reaction mixture was stirred at room-temperature for 16 hours. The reaction mixture was filtered and the filter cake was washed with ethyl acetate (100 mL×2). The filtrate was concentrated under reduced pressure. The resulting residue was purified by FCC (SiO₂, eluent: petroleum ether:ethyl acetate=1:0 to 5:1) to afford the title compound (10.8 g, 90%) colorless oil. MS (ESI): mass calcd. for C₁₄H₁₇NO₃ 247.1; m/z found 247.9 [M+H]⁺. ¹H NMR (400 MHz, DMSO-d₆) δ 7.38-7.28 (m, 4H), 4.03 (d, J=13.6 Hz, 1H), 3.92-3.72 (m, 7H), 3.57-3.44 (m, 2H), 2.60-2.50 (m, 1H), 2.27 (t, J=2.4 Hz, 1H).

Step B. Methyl 4-benzyl-6-methylenemorpholine-3-carboxylate. Ag₂CO₃ (11 g, 39.9 mmol) was added to a solution of methyl 2-(benzyl(prop-2-yn-1-yl)amino)-3-hydroxypropanoate (9.8 g, 39.6 mmol) in toluene (150 mL). The reaction mixture was stirred at room-temperature for 12 hours. The reaction mixture was filtered and the filter cake was washed with ethyl acetate (200 mL×2). The filtrate was concentrated under reduced pressure. The resulting residue was purified by FCC (SiO₂, eluent: petroleum ether:ethyl acetate=1:0 to 20:1) to afford the title compound (5.7 g, 51%) as colorless oil. MS (ESI): mass calcd. for C₁₄H₁₇NO₃ 247.1; m/z found 247.9 [M+H]⁺. ¹H NMR (400 MHz, DMSO-d₆) δ 7.41-7.28 (m, 5H), 4.44 (s, 1H), 4.24-4.12 (m, 2H), 4.08-3.87 (m, 2H), 3.78-3.61 (m, 5H), 3.42 (t, J=4.4 Hz, 1H), 3.03 (d, J=13.6 Hz, 1H).

Step C. Methyl 7-benzyl-1, 1-difluoro-4-oxa-7-azaspiro[2.5]octane-6-carboxylate. NaI (1.74 g, 11.6 mmol) and TMSCF₃ (8.9 mL, 60.7 mmol) were added to a solution consisting of methyl 4-benzyl-6-methylenemorpholine-3-carboxylate (5.7 g, 23.1 mmol) in dry THF (80 mL). The reaction mixture was stirred at 70° C. for 3 hours. The reaction mixture was cooled to room-temperature, quenched with sat·Na₂S₂O₃ (200 mL), and diluted with ethyl acetate (150 mL). The layers were separated and the aqueous layer was extracted with ethyl acetate (100 mL×2). The combined organic extracts were washed with brine (150 mL), dried over Na₂SO₄, filtered, and concentrated under reduced pressure to give a residue. The resulting residue was purified by FCC (SiO₂, eluent: petroleum ether:ethyl acetate=1:0 to 20:1) to afford the title compound, which was further purified by preparative HPLC using a Boston Uni C18, 40 mm×150 mm×5 m column (eluent: 48% to 78% (v/v) CH₃CN and H₂O with 0.225% HCOOH) to afford the title compound (1.27 g, 27%) as yellow oil. MS (ESI): mass calcd. For C₁₅H₁₇F₂NO₃ 297.1; m/z found 297.9 [M+H]⁺. ¹H NMR (400 MHz, DMSO-d₆) δ 7.38-7.27 (m, 5H), 4.23-4.09 (m, 1H), 4.02-3.93 (m, 2H), 3.88-3.67 (m, 4H), 3.51-3.28 (m, 2H), 2.56-2.30 (m, 1H), 1.51-1.42 (m, 1H), 1.35-1.27 (m, 1H).

Step D. Methyl 1, 1-difluoro-4-oxa-7-azaspiro [2.5] octane-6-carboxylate. A solution of methyl 7-benzyl-1, 1-difluoro-4-oxa-7-azaspiro[2.5]octane-6-carboxylate (1.7 g, 5.72 mmol), wet Pd(OH)₂ (900 mg, 20%), in THF (15 mL), was stirred under H₂ (15 psi) at room-temperature for 3 hours. The suspension was filtered through a pad of Celite® and the pad washed with THF (50 mL×2). The filtrate was concentrated to dryness under reduced pressure to afford the title product (1.2 g, crude) as a brown oil. MS (ESI): mass calcd. for C₈H₁₁F₂NO₃ 207.1; m/z found 207.9 [M+H]⁺.

Step E. 1, 1-Difluoro-4-oxa-7-azaspiro[2.5]octane-6-carboxylic acid. NaOH (467 mg, 11.7 mmol) was added to a solution of methyl 1, 1-difluoro-4-oxa-7-azaspiro[2.5]octane-6-carboxylate (1.20 g, 5.79 mmol), in MeOH (10 mL), and H₂O (1 mL). The reaction mixture was stirred at room-temperature for 2 hours. The reaction mixture was concentrated under reduced pressure to give a residue, which was dissolved with H₂O (15 mL) and adjusted to pH=6 with 1 M HCl. The aqueous phase was washed with ethyl acetate (10 mL×2). The aqueous layer (1 g, crude in water (15 mL)) was used to next step directly without further workup. MS (ESI): mass calcd. for C₇H₉F₂NO₃ 193.1; m/z found 193.9 [M+H]⁺.

Step F: 3′-Bromo-2,2-difluoro-2′-(5-fluoropyridin-2-yl)-4′H,7′H-spiro[cyclopropane-1,6′-pyrazolo[5,1-c][1,4]oxazine]. The title compound was prepared in a manner analogous to (R)-3-bromo-5-fluoro-2-(4-fluorophenyl)-5,6-dihydro-4H-pyrrolo[1,2-b]pyrazole (Intermediate 150) Steps A-C; using 1, 1-difluoro-4-oxa-7-azaspiro[2.5]octane-6-carboxylic acid instead of (2S,4R)-4-fluoropyrrolidine-2-carboxylic acid in Step A; and using 2-ethynyl-5-fluoropyridine instead of 4-fluorophenylacetylene in Step B. MS (ESI): mass calcd. for C₁₃H₁₀F₃N₃O 281.1; m/z found 281.9 [M+H]⁺. ¹H NMR (400 MHz, DMSO-d₆) δ 8.52-8.46 (m, 1H), 7.97-7.87 (m, 1H), 7.50-7.37 (m, 1H), 6.69-6.56 (m, 1H), 5.09-4.90 (m, 2H), 4.43-4.34 (m, 2H), 1.93-1.61 (m, Intermediate 192: 3-Bromo-2-(5-fluoropyridin-2-yl)-7-methyl-6,7-dihydro-5H-pyrazolo[5,1-b][1,3]oxazine

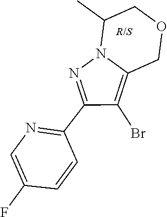 [SMILES: CC1COCc2c(Br)c(-c3ccc(F)cn3)nn21]

The title compound was prepared in a manner analogous to 3-bromo-2-(5-fluoropyridin-2-yl)-6,7-dihydro-5H-pyrazolo[5,1-b][1,3]oxazine (Intermediate 119, Steps A-E), except using 1,3-dibromobutane instead of 1-bromo-3-chloropropane in Step D. MS (ESI): mass calcd. for C₁₂H₁₁BrN₃O 311.0; m/z found 312.0 [M+H]⁺. ¹H NMR (400 MHz, CDCl₃): δ 8.57 (d, J=3.2 Hz, 1H), 7.97 (dd, J=4.4, 8.4 Hz, 1H), 7.48-7.42 (m, 1H), 4.50-4.33 (m, 3H), 2.44-2.35 (m, 1H), 2.10-2.01 (m, 1H), 1.65 (d, J=6.4 Hz, 3H); ¹⁹F NMR (376 MHz, CDCl₃): −127.70 (br s, 1F).

### Intermediate 193: 3-Bromo-6,6-difluoro-2-(4-fluorophenyl)-6,7-dihydro-5H-pyrazolo[5,1-b][1,3]oxazine

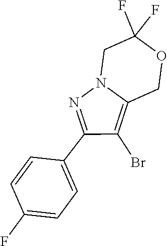 [SMILES: Fc1ccc(-c2nn3c(c2Br)COC(F)(F)C3)cc1]

Step A. 2,2-Difluoropropane-1,3-diyl dimethanesulfonate. MsCl (6.54 g, 57.1 mmol) was added dropwise to a 0° C. solution of 2,2-difluoropropane-1,3-diol (2.0 g, 18 mmol), and triethylamine (10 mL, 72 mmol), in dichloromethane (60 mL). The reaction mixture was stirred for at room-temperature 16 hours. The reaction mixture was quenched with water (100 mL) and extracted with dichloromethane (100 mL×2). The organic extracts were combined, dried over anhydrous Na₂SO₄, filtered and concentrated under reduced pressure to afford the title compound (4.0 g, 84%), which was used directly without further purification in the next step. ¹H NMR (400 MHz, CDCl₃): δ 4.45 (tt, J=2.5, 11.5 Hz, 4H), 3.21-3.03 (m, 6H).

Step B. 6,6-Difluoro-2-(4-fluorophenyl)-6,7-dihydro-5H-pyrazolo[5,1-b][1,3]oxazine. K₂CO₃ (1.72 g, 12.5 mmol) was added to a mixture of 5-(4-fluorophenyl)-1H-pyrazol-3(2H)-one (Intermediate 44 Step A, 500 mg, 2.81 mmol), and 2,2-difluoropropane-1,3-diyl dimethanesulfonate (833 mg, 3.11 mmol) in MeCN (20 mL). The reaction mixture was stirred at 90° C. for 48 hours. The reaction mixture was cooled to room-temperature, filtered and concentrated to dryness. The resulting residue was purified by FCC (SiO₂, euent: ethyl acetate: petroleum ether=1:10 to 1:5) to afford the title compound (250 mg, 35%) as white solid. MS (ESI): mass calcd. for C₁₂H₉F₃N₂O 254.1; m/z found 254.9 [M+H]⁺.

Step C. 3-Bromo-6,6-difluoro-2-(4-fluorophenyl)-6,7-dihydro-5H-pyrazolo[5,1-b][1,3]oxazine. NBS (176 mg, 0.989 mmol) was added to a mixture of 6,6-difluoro-2-(4-fluorophenyl)-6,7-dihydro-5H-pyrazolo[5,1-b][1,3]oxazine (250 mg, 0.983 mmol) in dichloromethane (5 mL). The reaction mixture was stirred at room-temperature for 30 mins. The reaction mixture was poured to H₂O (10 mL) and extracted with dichloromethane (10 mL×3). The organic extracts were combined, dried over anhydrous Na₂SO₄, filtered and concentrated to dryness. The resulting residue was purified by FCC (SiO₂, eluent: ethyl acetate:petroleum ether=1:10 to 1:3) to afford the title compound (300 mg, 92%) as yellow solid. MS (ESI): mass calcd. for C₁₂H₈BrF₃N₂O 332.0; m/z found 332.7 [M+H]⁺.

### Intermediate 194: 3-Bromo-2-(4-fluorophenyl)-6,6-dimethyl-6,7-dihydro-5H-pyrazolo[5,1-b][1,3]oxazine

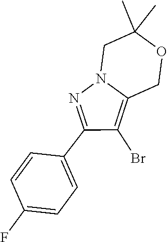 [SMILES: CC1(C)Cn2nc(-c3ccc(F)cc3)c(Br)c2CO1]

Step A. 2-(4-Fluorophenyl)-6,6-dimethyl-6,7-dihydro-5H-pyrazolo[5,1-b][1,3]oxazine. 1,3-Dibromo-2,2-dimethylpropane (1.09 g, 4.74 mmol) was added to a solution of 5-(4-fluorophenyl)-1,2-dihydro-3H-pyrazol-3-one (Intermediate 127, Step A, 600 mg, 3.37 mmol), and K₂CO₃ (1.4 g, 10 mmol), in DMF (10 mL). The resultant mixture was stirred at 120° C. for 16 hours. The reaction mixture was poured into aq. LiCl (15 mL, 1 M) and extracted with ethyl acetate (15 mL×3). The combined organic extracts were dried over anhydrous Na₂SO₄, filtered, and concentrated to dryness under reduced pressure to give the title compound (700 mg) as a yellow solid, which was used in the next step without further purification. MS (ESI): mass calcd. for C₁₄H₁₅FN₂O 246.1; m/z found 246.9 [M+H]⁺.

Step B. 3-Bromo-2-(4-fluorophenyl)-6,6-dimethyl-6,7-dihydro-5H-pyrazolo[5,1-b][1,3]oxazine. Trifluoromethanesulfonic acid (0.25 mL, 2.8 mmol) was added dropwise to a 0° C. (ice/water) solution of 2-(4-fluorophenyl)-6,6-dimethyl-6,7-dihydro-5H-pyrazolo[5,1-b][1,3]oxazine (700 mg), and NBS (612 mg, 3.44 mmol) in dichloromethane (10 mL). The reaction mixture was stirred at 0° C. (ice/water) for 1 hour. The reaction mixture was quenched with H₂O (15 mL) and extracted with dichloromethane (15 mL×2). The combined organic extracts were dried over anhydrous Na₂SO₄, filtered, and concentrated to dryness under reduced pressure. The resulting residue was purified by FCC (SiO₂, eluent: petroleum ether:ethyl acetate=1:0 to 2:1) to afford the title compound (600 mg) as a yellow solid. MS (ESI): mass calcd. for C₁₄H₁₄BrFN₂O 324.0; m/z found 324.9 [M+H]⁺.

### Intermediate 195: 3′-Bromo-2′-(4-fluorophenyl)-5′H,7′H-spiro[cyclopropane-1,6′-pyrazolo[5,1-b][1,3]oxazine]

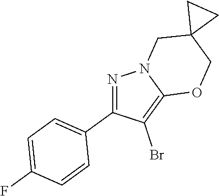 [SMILES: Fc1ccc(-c2nn3c(c2Br)OCC2(CC2)C3)cc1]

Step A: Cyclopropane-1,1-diylbis(methylene) dimethanesulfonate. To cyclopropane-1,1-diyldimethanol (10 g, 98 mmol) in DCM (200 mL) was added triethylamine (54.4 mL, 392 mmol). The reaction mixture was cooled to 0° C. and methanesulfonyl chloride (22.7 mL, 294 mmol) was added dropwise over 1 hour. The reaction was allowed to warm to room temperature and stirred for an additional 2 hours before water was added and the resulting solid was collected via filtration to provide the title compound (14.48 g, 57%). ¹H NMR (400 MHz, CD₃OD) δ 4.22 (s, 4H), 3.12 (s, 6H), 0.85 (s, 4H).

Step B: 2′-(4-Fluorophenyl)-5′H,7′H-spiro[cyclopropane-1,6′-pyrazolo[5,1-b][1,3]oxazine]. To a solution of 5-(4-fluorophenyl)-1,2-dihydro-3H-pyrazol-3-one (Intermediate 127, Step A, 3.07 g, 12.92 mmol), in DMF (31 mL) was added Cs₂CO₃ (12.6 g, 38.8 mmol). Cyclopropane-1,1-diylbis(methylene) dimethanesulfonate (4.01 g, 15.51 mmol) was added to the reaction mixture and the reaction mixture was heated at 50° C. overnight. The reaction mixture was cooled and filtered; the resulting solids were washed with EtOAc. The filtrate was diluted with water and extracted with EtOAc (2×). The combined organics were dried with sodium sulfate, filtered, and concentrated to produce a brown oil (13.7 g). DCM was then added causing a precipitate to form which was isolated via filtration. The solids were washed with a minimum amount of DCM and dried under high-vac overnight to provide the title compound (963 mg, 30%). MS (ESI): mass calcd. for C₁₄H₁₃FN₂O, 244.1; m/z found, 245.1 [M+H]⁺.

Step C: 3′-Bromo-2′-(4-fluorophenyl)-5′H,7′H-spiro[cyclopropane-1,6′-pyrazolo[5,1-b][1,3]oxazine]. To a cooled (0° C.) solution of 2′-(4-fluorophenyl)-5′H,7′H-spiro[cyclopropane-1,6′-pyrazolo[5,1-b][1,3]oxazine] (960 mg, 3.93 mmol) in DCM (22 mL) was added N-bromosuccinimide (707 mg, 3.93 mmol) in a single portion. After 10 minutes the solvent was removed under reduced pressure. The resulting residue was purified by FCC (SiO₂, Hex/EtOAc) to provide the title compound (1.07 g, 84%) as a brownish solid. MS (ESI): mass calcd. for C₁₄H₁₂BrFN₂O, 322.0; m/z found, 323.0 [M+H]⁺. ¹H NMR (500 MHz, CD₃OD) δ 7.91-7.69 (m, 2H), 7.25-7.01 (m, 2H), 4.17 (s, 2H), 4.02 (s, 2H), 1.00-0.75 (m, 4H).

### Intermediate 196: 3′-Bromo-2′-(5-fluoropyridin-2-yl)-5′H,7′H-spiro[cyclopropane-1,6′-pyrazolo[5,1-b][1,3]oxazine]

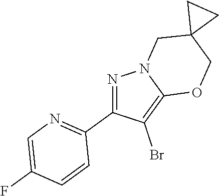 [SMILES: Fc1ccc(-c2nn3c(c2Br)OCC2(CC2)C3)nc1]

Step A: 2′-(5-Fluoropyridin-2-yl)-5′H,7′H-spiro[cyclopropane-1,6′-pyrazolo[5,1-b][1,3]oxazine]. A solution of 5-(5-fluoropyridin-2-yl)-1,2-dihydro-3H-pyrazol-3-one (Intermediate 119, Step C, 608 mg, 3.394 mmol), cyclopropane-1,1-diylbis(methylene) dimethanesulfonate (Intermediate 195, product from Step B, 1.052 g, 4.072 mmol), and Cs₂CO₃ (3.317 g, 10.181 mmol) in DMF was heated at 60° C. for 18 hours. The reaction mixture was cooled, brine was added to the reaction mixture, and the reaction mixture was extracted with EtOAc. The combined organic extracts were concentrated under reduced pressure. The resulting residue was purified by FCC (hex/(EtOAc/10% MeOH)) to provide the title compound (563 mg, 68%). MS (ESI): mass calcd. for C₁₃H₁₂FN₃O, 245.1; m/z found, 246.1 [M+H]⁺.

Step B: Bromo-2′-(5-fluoropyridin-2-yl)-5′H,7′H-spiro[cyclopropane-1,6′-pyrazolo[5,1-b][1,3]oxazine]. To a cooled (0° C.) solution of 2′-(5-fluoropyridin-2-yl)-5′H,7′H-spiro[cyclopropane-1,6′-pyrazolo[5,1-b][1,3]oxazine] (563 mg, 2.296 mmol) in DMF (13.9 mL) was added N-bromosuccinimide (425 mg, 2.388 mmol) in portions. The reaction mixture was allowed to warm to room-temperature over 2.5 hours and then diluted with brine and extracted with EtOAc. The organics were dried with sodium sulfate, filtered, and concentrated. The resulting residue was purified by FCC (SiO₂, hex/(EtOAc/10% MeOH)) to provide the title compound (587 mg, 79%) as a yellow solid. MS (ESI): mass calcd. for C₁₃H₁₁BrFN₃O, 323.0; m/z found, 324.0 [M+H]⁺.

### Intermediate 197: 3′-Bromo-2,2-difluoro-2′-(4-fluorophenyl)-5′H,7′H-spiro[cyclopropane-1,6′-pyrazolo[5,1-b][1,3]oxazine]

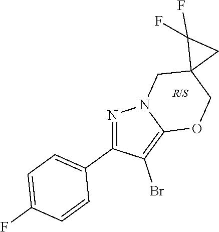 [SMILES: Fc1ccc(-c2nn3c(c2Br)OCC2(C3)CC2(F)F)cc1]

Step A: (2,2-Difluorocyclopropane-1,1-diyl)bis(methylene) bis(4-methylbenzenesulfonate). To a cooled solution (0° C.) of (2,2-difluorocyclopropane-1,1-diyl)dimethanol (1 g, 7.2 mmol) in DCM (5 mL), and triethylamine (5.0 mL, 36.2 mmol) was added tosyl chloride (3.439 g, 18.10 mmol). The reaction was removed from the cold bath and allowed to stir for 1 hour. The reaction mixture was then washed with water and extracted with EtOAc. The organics were dried with sodium sulfate, filtered, concentrated under reduced pressure The resulting residue was purified by FCC (hex/EtOAc) to provide the title compound (1.36 g, 42%).

Step B: 2,2-Difluoro-2′-(4-fluorophenyl)-5′H,7′H-spiro[cyclopropane-1,6′-pyrazolo[5,1-b][1,3]oxazine]. A solution of 5-(4-fluorophenyl)-1,2-dihydro-3H-pyrazol-3-one (Intermediate 119, Step C, 500 mg, 2.806 mmol), (2,2-difluorocyclopropane-1,1-diyl)bis(methylene) bis(4-methylbenzenesulfonate) (Intermediate 197, product from Step A, 1.364 g, 3.055 mmol), and Cs₂CO₃ (2.286 g, 7.016 mmol) in DMF (8 mL) was heated at 68° C. for 20 hours. Water was then added and the reaction mixture was extracted with EtOAc, concentrated under reduced pressure. The resulting residue was purified by FCC (hex/(10% MeOH in EtOAc)) to provide the title compound (487 mg, 62%). MS (ESI): mass calcd. for C₁₄H₁₁F₃N₂O, 280.1; m/z found, 281.1 [M+H]⁺.

Step C: 3′-Bromo-2,2-difluoro-2′-(4-fluorophenyl)-5′H,7′H-spiro[cyclopropane-1,6′-pyrazolo[5,1-b][1,3]oxazine]. To a solution of 2,2-difluoro-2′-(4-fluorophenyl)-5′H,7′H-spiro[cyclopropane-1,6′-pyrazolo[5,1-b][1,3]oxazine] (487 mg, 1.738 mmol) in DMF was added N-bromosuccinimide (325 mg, 1.825 mmol) in three portions. The reaction mixture was stirred at room temperature for 2 hours and then diluted with brine and extracted with EtOAc. The organics were concentrated under reduced pressure. The resulting residue was purified by FCC (SiO₂, hex/(10% MeOH in EtOAc)) to provide the title compound (580 mg, 93%). MS (ESI): mass calcd. for C₁₄H₁₀BrF₃N₂O, 358.0; m/z found, 359.0 [M+H]⁺.

### Intermediate 198: 3′-Bromo-2,2-difluoro-2′-(5-fluoropyridin-2-yl)-5′H,7′H-spiro[cyclopropane-1,6′-pyrazolo[5,1-b][1,3]oxazine]

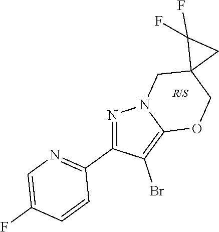 [SMILES: Fc1ccc(-c2nn3c(c2Br)OCC2(C3)CC2(F)F)nc1]

Step A: 2,2-difluoro-2′-(5-fluoropyridin-2-yl)-5′H,7′H-spiro[cyclopropane-1,6′-pyrazolo[5,1-b][1,3]oxazine]. A solution of 5-(5-fluoropyridin-2-yl)-1,2-dihydro-3H-pyrazol-3-one (Intermediate 119, Step C, 435 mg, 2.428 mmol), (2,2-difluorocyclopropane-1,1-diyl)bis(methylene) bis(4-methylbenzenesulfonate) (Intermediate 197 product from Step A) 1.18 g, 2.64 mmol), and Cs₂CO₃ (1.948 g, 5.979 mmol) in DMF (7 mL) was heated at 68° C. for 20 hours. Water was then added and the reaction mixture was extracted with EtOAc. The combined organics were concentrated under reduced pressure. The resulting residue was purified by FCC (hex/(EtOAc/10% MeOH)) to provide the title compound (483 mg, 71%). MS (ESI): mass calcd. for C₁₃H₁₀F₃N₃O, 281.1; m/z found, 282.0 [M+H]⁺.

Step B: 3′-Bromo-2,2-difluoro-2′-(5-fluoropyridin-2-yl)-5′H,7′H-spiro[cyclopropane-1,6′-pyrazolo[5,1-b][1,3]oxazine]. To a cooled to 0° C. solution of 2,2-difluoro-2′-(5-fluoropyridin-2-yl)-5′H,7′H-spiro[cyclopropane-1,6′-pyrazolo[5,1-b][1,3]oxazine] (432 mg, 1.536 mmol) in DMF (2.2 mL) was added N-bromosuccinimide (287 mg, 1.613 mmol) in portions. The reaction mixture was allowed to warm to room-temperature over 1.5 hours and then diluted with water and extracted with EtOAc. The organics were dried with sodium sulfate, filtered, and concentrated under reduced pressure. The resulting residue was purified by FCC (SiO₂, hex/(EtOAc/10% MeOH)) to provide the title compound (410 mg, 74%). MS (ESI): mass calcd. for C₁₃H₉BrF₃N₃O, 359.0; m/z found, 360.0 [M+H]⁺.

### Intermediate 199: 3-Bromo-2-(5-fluoropyridin-2-yl)-7-methyl-7,8-dihydro-4H,6H-pyrazolo[5,1-c][1,4]oxazepin-7-ol

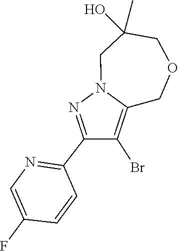 [SMILES: CC1(O)COCc2c(Br)c(-c3ccc(F)cn3)nn2C1]

Step A: 2-(5-Fluoropyridin-2-yl)-7-methyl-7,8-dihydro-4H,6H-pyrazolo[5,1-c][1,4]oxazepin-7-ol. To a solution of tert-butyl-[[3-(5-fluoro-2-pyridyl)-1 h-pyrazol-5-yl]methoxy]-dimethyl-silane (Intermediate 35, 200 mg, 0.65 mmol) and Cs₂CO₃ (636 mg, 1.95 mmol, 3 eq.) in DMF (3.3 mL) was added 2-(chloromethyl)-2-methyloxirane (79 μL, d=1.057 g/ml, 0.78 mmol, 1.2 eq.). The resulting brown suspension was stirred at room-temperature for 18 hrs; then heated at 50° C. for 2 hrs followed by microwave irradiation at 120° C. for 10 minutes. The reaction mixture was diluted with H₂O (5 mL), extracted with EtOAc (5 mL×2), the aqueous layer was further extracted with DCM/IPA (4/1, 5 mL×2). The combined organic layer was dried over anhydrous Na₂SO₄, filtered, and concentrated under reduced. The resulting residue was purified by FCC (SiO₂, eluent: EtOAc: DCM=0-50%) to afford the title compound (40 mg, 23%) as a white solid. MS (ESI): mass calcd. for C₁₃H₁₄FN₃O₂ 263.1; m/z found 264.1 [M+H]⁺. ¹H NMR (400 MHz, CD₃OD) δ 8.42 (dt, J=3.0, 0.7 Hz, 1H), 8.03-7.93 (m, 1H), 7.63 (td, J=8.6, 2.9 Hz, 1H), 6.76 (s, 1H), 4.70 (d, J=2.3 Hz, 2H), 4.41 (d, J=0.8 Hz, 2H), 3.79 (dd, J=1.8, 0.9 Hz, 2H), 1.13 (s, 3H).

Step B: 3-Bromo-2-(5-fluoropyridin-2-yl)-7-methyl-7,8-dihydro-4H,6H-pyrazolo[5,1-c][1,4]oxazepin-7-ol. To a solution of 2-(5-fluoropyridin-2-yl)-7-methyl-7,8-dihydro-4H,6H-pyrazolo[5,1-c][1,4]oxazepin-7-ol (57 mg, 0.217 mmol, 1 eq.) in DCM (1.2 mL) was added NBS (39 mg, 0.22 mmol, 1 eq.) in one portion at room temperature. After 1 hr another 1 eq of NBS (39 mg, 0.22 mmol) was added to the reaction mixture, and the reaction mixture was stirred at room temperature for an additional 2 hrs. To the reaction mixture was added DMF (0.5 mL) and another 1 eq. of NBS (39 mg, 0.22 mmol). The reaction mixture was stirred at room temperature for another hour. The reaction mixture was concentrated under reduced pressure. The resulting residue was purified by FCC (SiO₂, EtOAc/DCM 0-50%) to afford the title compound 83 mg (100%, 89% purity) as a colorless oil. MS (ESI): mass calcd. for C₁₃H₁₃BrFN₃O₂ 341.0; m/z found 342.0 [M+H]⁺.

### Intermediate 200: cis-3-Bromo-2-(5-fluoropyridin-2-yl)-5a,6,6a,7-tetrahydro-5H-cyclopropa[e]pyrazolo[5,1-b][1,3]oxazepane

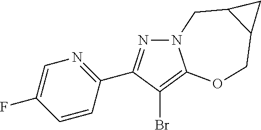 [SMILES: Fc1ccc(-c2nn3c(c2Br)OCC2CC2C3)nc1]

Step A: cis-Cyclopropane-1,2-diyldimethanol. To a cooled solution (0° C.) of 3-oxabicyclo[3.1.0]hexane-2,4-dione (10.0 g, 89.2 mmol) in THF (200 mL) was added LiAlH₄ (8.50 g, 224 mmol) in portions under N₂. The reaction mixture was stirred at 70° C. for 16 hours. The reaction mixture was cooled to room-temperature, and the reaction mixture was quenched with water (9 mL), aq. NaOH (2 M, 9 mL), then H₂O (18 mL). The resulting solution was stirred at room-temperature for 1 hour. The reaction mixture was filtered and concentrated to dryness under reduced pressure to give the title compound (7.5 g, 82%) as a yellow oil. ¹H NMR (400 MHz, CDCl₃): δ 4.00-3.78 (m, 2H), 3.06 (t, J=10.4 Hz, 2H), 2.48 (s, 2H), 1.23-1.04 (m, 2H), 0.76-0.52 (m, 1H), 0.02 (q, J=5.2 Hz, 1H).

Step B. cis-1,2-Bis(bromomethyl)cyclopropane. To a solution consisting of cyclopropane-1,2-diyldimethanol (2.00 g, 19.6 mmol) in dry dichloromethane (100 mL) under argon, was added PPh₃ (10.8 g, 41.2 mmol). The reaction mixture was cooled to 5° C. N-bromosuccinimide (7.3 g, 41 mmol) was added in portions at 0-5° C. The reaction mixture was stirred at 22° C. for 18 hours. The reaction mixture was concentrated under reduced pressure. The residue was diluted with petroleum ether (25 mL), the pooled extracts were cooled at 5° C., and the precipitated triphenylphosphine oxide was filtered off. The filtrate was evaporated to dryness to afford the title compound (1.5 g, 34%), which was directly used in the subsequent transformations without further purification. ¹H NMR (400 MHz, CDCl₃) δ 3.55-3.40 (m, 4H), 1.70-1.60 (m, 2H), 1.15-1.11 (m, 1H), 0.42 (d, J=5.7 Hz, 1H).

Step C: cis-3-Bromo-2-(5-fluoropyridin-2-yl)-5a,6,6a,7-tetrahydro-5H-cyclopropa[e]pyrazolo[5,1-b][1,3]oxazepane. The title compound was prepared in a manner analogous to 3-bromo-2-(5-fluoropyridin-2-yl)-6,7-dihydro-5H-pyrazolo[5,1-b][1,3]oxazine (Intermediate 119), except using cis-1,2-bis(bromomethyl)cyclopropane (Intermediate 200, product from Step B) instead of 1-bromo-3-chloropropane. MS (ESI): mass calcd. for C₁₃H₁₁BrFN₃O 323.0; m/z found 323.8 [M+H]⁺.

### Intermediate 201: cis-2-(5-Fluoropyridin-2-yl)-3-iodo-5a,6,6a,7-tetrahydro-5H-cyclopropa[e]pyrazolo[5,1-b][1,3]oxazepine

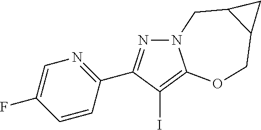 [SMILES: Fc1ccc(-c2nn3c(c2I)OCC2CC2C3)nc1]

The title compound was prepared in a manner analogous to 3-bromo-2-(5-fluoropyridin-2-yl)-6,7-dihydro-5H-pyrazolo[5,1-b][1,3]oxazine (Intermediate 119, Steps A-E), except using cis-1,2-bis(bromomethyl)cyclopropane (Intermediate 200 product from Step B) instead of 1-bromo-3-chloropropane in Step D and using NIS instead of NBS in Step E. MS (ESI): mass calcd. for C₁₃H₁₁FIN₃O 371.0; m/z found 371.9 [M+H]⁺.

### Intermediate 202: 4-Iodo-3-methyl-1-((2-(trimethylsilyl)ethoxy)methyl)-1H-pyrazolo[3,4-b]pyridine

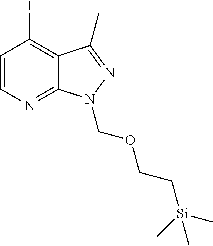 [SMILES: Cc1nn(COCC[Si](C)(C)C)c2nccc(I)c12]

Step A: 4-Iodobut-3-yn-2-one. To a solution of 4-(trimethylsilyl)but-3-yn-2-one (50 g, 356 mmol) in MeCN (600 mL) was added AgF (47.5 g, 374 mmol). The reaction mixture was stirred for 30 minutes. NIS (84 g, 374 mmol) was added slowly (approx. half of the amount) to the reaction mixture, and the reaction exothermed to 25° C. The rest of the NIS was then added maintaining the temperature about 25° C., and the reaction mixture was stirred at RT for 2 h. Additional AgF (2 g, 16 mmol) and NIS (2 g, 8.9 mmol) were then added and the resulting mixture was stirred for 20 min before additional AgF (0.5 g, 3.9 mmol) was added. After 20 mins the reaction mixture was diluted with MTBE (600 mL) and water (400 mL) and the layers separated. The MTBE layer was washed with water (300 mL) and the organic layer was dried over MgSO₄, filtered, and partially concentrated under reduced pressure to produce a hazy solution. Additional MgSO₄ was then added and the mixture was filtered to give a clear reddish solution which was further concentrated to yield 60 g of material. 50 g of this material was stored in the freezer and upon thawing the product had solidified. The solids were diluted with 10:1 Hex/EtOAc (100 mL) and stirred at 0° C. for 20 min. The solids were then filtered, rinsed with cold 10:1 Hex/EA, placed on high-vac to yield the title compound (19.6 g, 94% purity, 27% yield). ¹H NMR (400 MHz, CDCl₃): δ 2.35 (s, 3H).

Step B. 4-Iodo-3-methyl-1H-pyrazolo[3,4-b]pyridine. To a solution of 2-hydrazineylpyrimidine (4.0 g, 36 mmol) in THF (150 mL) was added TFA (0.115 mL, 1.5 mmol) followed by 4-iodobut-3-yn-2-one (Step A, 6.2 g, 30 mmol) in THF (10 mL+2 mL rinse). The reaction mixture was stirred for 30 min at RT. TFAA (12.5 mL, 90 mmol) was added followed by pentanone (9.5 mL, 90 mmol), and the reaction mixture was heated to 60° C. for one hour. The reaction mixture was diluted with EtOAc (800 mL) and washed with sat aq Na₂CO₃ (200 mL). The organic layer was separated, dried over MgSO₄, filtered, preabsorbed onto Celite®, and purified by FCC (SiO₂, Hex/EA 0-60%) to the title compound (2.29 g). 1.9 g of the title compound was then mixed with silica as a slurry in ACN and stirred for 1 h before being filtered to afford the title compound as light brown solid (1.6 g, 20.6%). MS (ESI): mass calcd. for C₇H₆IN₃ 259.0; m/z found 260.0 [M+H]⁺.

Step C. 4-Iodo-3-methyl-1-((2-(trimethylsilyl)ethoxy)methyl)-1H-pyrazolo[3,4-b]pyridine. To a cooled solution (0° C.) of 4-iodo-3-methyl-1H-pyrazolo[3,4-b]pyridine (2 g, 7.7 mmol) in DMF (2 mL) was added NaHMDS (2M in THF, 7.7 ml, 15.4 mmol). The reaction mixture was stirred for 5 mins, and SEM-Cl (2.57 g, 15.4 mmol) was added. The reaction mixture was stirred for 30 min at 0° C. after which LCMS analysis showed the reaction was complete. The reaction mixture was then diluted with water and extracted with EtOAc. The combined organic layers were dried over MgSO₄, filtered, and concentrated under reduced pressure. The resulting residue was purified by FCC (SiO₂, Hex/EtOAc 0-20% to deliver the title compound as an orange solid (2.04 g, 68%). MS (ESI): mass calcd. for C₁₃H₂₀IN₃OSi 389.0; m/z found 390.0 [M+H]⁺. ¹H NMR (500 MHz, CDCl₃): δ 8.05 (d, J=4.8 Hz, 1H), 7.59 (d, J=4.8 Hz, 1H), 5.77 (s, 2H), 3.66-3.59 (m, 2H), 2.73 (s, 3H), 0.98-0.89 (m, 2H), −0.05 (s, 9H).

### Intermediate 203: 4-Chloro-3-methyl-1-((2-(trimethylsilyl)ethoxy)methyl)-1H-pyrazolo[3,4-b]pyridine

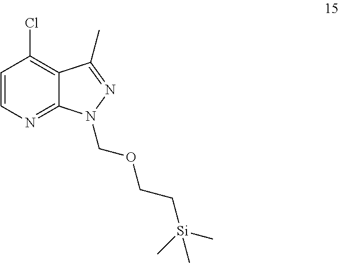 [SMILES: Cc1nn(COCC[Si](C)(C)C)c2nccc(Cl)c12]

Step A: 4-Chloro-3-iodo-1H-pyrazolo[3,4-b]pyridine. NIS (38.0 g, 169 mmol) was added to a solution of 4-chloro-1H-pyrazolo[3,4-b]pyridine (20.0 g, 130 mmol) and DMF (200 mL). The reaction mixture was stirred at 80° C. for 8 h. The reaction mixture was cooled to room-temperature and poured into 300 mL of water. The resulting suspension was isolated via filtration. The filter cake was washed with water (100 mL×3) and ACN (50 mL×3). The resulting solids were dried under reduced pressure to afford the title compound (30.0 g, 83%) as a yellow solid.

Step B: 4-Chloro-3-iodo-1-((2-(trimethylsilyl)ethoxy)methyl)-1H-pyrazolo[3,4-b]pyridine. Sodium hydride in mineral oil (3.72 g, 60% purity, 93.0 mmol) was added in portions to a 0° C. (ice/water) solution of 4-chloro-3-iodo-1H-pyrazolo[3,4-b]pyridine (20.0 g, 71.6 mmol) and THF (200 mL). The reaction mixture was stirred at 0° C. for 30 mins. SEMCl (19.0 mL, 107 mmol) was added to the above mixture at 0° C. The resultant mixture was stirred for 3 hours. The reaction mixture was warmed to room-temperature, poured into sat·NH₄Cl (100 mL) and extracted with ethyl acetate (200 mL×3). The combined organic extracts were washed with brine (100 mL×3), dried over anhydrous Na₂SO₄, filtered, and concentrated to dryness under reduced pressure. The resulting residue was purified by FCC (SiO₂, petroleum ether:ethyl acetate=1:0 to 95:5) to afford the title compound (10 g, 34%) as a colorless oil. ¹H NMR (400 MHz, DMSO-d₆) δ 8.57 (d, J=4.8 Hz, 1H), 7.45 (d, J=5.0 Hz, 1H), 5.75 (s, 2H), 3.58 (s, 2H), 0.81 (s, 2H), −0.03-−0.21 (m, 9H). MS (ESI): mass calcd. for C₁₂H₁₇ClIN₃OSi 409.0; m/z found 409.9 [M+H]⁺.

Step C: 4-Chloro-3-methyl-1-((2-(trimethylsilyl)ethoxy)methyl)-1H-pyrazolo[3,4-b]pyridine. To a mixture of 4-chloro-3-iodo-1-((2-(trimethylsilyl)ethoxy)methyl)-1H-pyrazolo[3,4-b]pyridine (8.00 g, 19.5 mmol), 2,4,6-trimethyl-1,3,5,2,4,6-trioxatriborinane (11.0 mL, 39.3 mmol), and Cs₂CO₃ (19.0 g, 58.3 mmol) in 1,4-dioxane (80 mL), and H₂O (20 mL), sparged with Ar for 5 minutes, was added Pd(PPh₃)₄ (2.24 g, 1.94 mmol). The reaction mixture was sparged with Ar for another 5 minutes and then stirred at 80° C. for 8 hours. The reaction mixture was cooled to room-temperature, diluted with H₂O (100 mL) and extracted with ethyl acetate (250 mL×3). The combined organic extracts were dried over anhydrous Na₂SO₄, filtered, and concentrated to dryness under reduced pressure. The resulting residue was purified by FCC (SiO₂, eluent: petroleum ether:ethyl acetate=1:0 to 9:1) to afford the product (3.9 g, 59%) as a brown oil. MS (ESI): mass calcd. for C₁₃H₂₀ClN₃OSi 297.1; m/z found 298.1 [M+H]⁺. ¹H NMR (400 MHz, CDCl₃): δ 8.39 (d, J=4.9 Hz, 1H), 7.11 (d, J=4.9 Hz, 1H), 5.79 (s, 2H), 3.66-3.57 (m, 2H), 2.75 (s, 3H), 0.97-0.92 (m, 2H), −0.03-−0.06 (m, 9H).

### Intermediate 204: 5-Fluoro-4-iodo-1-((2-(trimethylsilyl)ethoxy)methyl)-1H-pyrazolo[3,4-b]pyridine

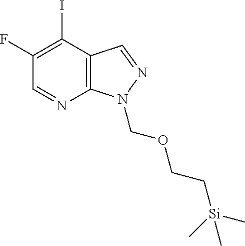 [SMILES: C[Si](C)(C)CCOCn1ncc2c(I)c(F)cnc21]

Step A: 3-(Trimethylsilyl)propiolaldehyde. To a solution of trimethylsilylacetylene (650 g, 6.62 mol), in THF (6.50 L), purged and maintained under an inert atmosphere of nitrogen, was added n-BuLi (3.04 L, 2.5 mol/L) dropwise at −70° C. The reaction mixture was stirred for 1 h at −70 to −60° C. DMF (967 g, 13.2 mol,) was added to the reaction mixture dropwise at −45° C. The reaction mixture was stirred for an additional 1 h at −45 to −30° C. The reaction mixture was quenched with aqueous citric·acid sol'n (5 L, 3 wt %) and extracted with MTBE (3×6 L). The organic layers were separated and washed with brine (2×3 L). The organics layer was dried over anhydrous sodium sulfate, filtered, and concentrated under reduced pressure to afford the title compound as a yellow oil (550 g, 66%). ¹H NMR (300 MHz, DMSO-d₆): δ 9.19 (s, 9H), 0.27 (s, 9H).

Step B: 3-Iodopropiolaldehyde. A solution of 3-(trimethylsilyl)propiolaldehyde (550.00 g, 4357.12 mmol, 1.00 equiv), and AgF (582.00 g, 4583.30 mmol, 1.05 equiv), in ACN (6.00 L), under an inert atmosphere of nitrogen was stirred for 30 min. To the reaction mixture was added NIS (1031.00 g, 4583.30 mmol, 1.05 equiv), portion-wise at 25° C. The reaction mixture was stirred for 2 h at room temperature. The resulting solids were filtered off. The reaction filtrate was diluted with MTBE (6 L) and quenched water/ice (4 L). The layers were separated and the organics dried over anhydrous sodium sulfate and concentrated at 20° C. to yield the title compound as a yellow solid (605 g, 77%). ¹H NMR (400 MHz, CDCl₃): 9.14 (s, 1H).

Step C: 5-Fluoro-2-hydrazineylpyrimidine. A solution of 2-chloro-5-fluoropyrimidine (260 g, 2.0 mol), EtOH (1.60 L), and hydrazine hydrate (1.50 L, 30.9 mol), under an inert atmosphere of nitrogen, was stirred for 1 h at 60° C. The reaction mixture was cooled to 25° C. and concentrated under reduced pressure. The reaction mixture was quenched with water/ice (1 L). The resulting solids were collected by filtration and dried in an oven to provide the title compound as a white solid (200 g, 80%). MS (ESI): mass calcd. for C₄H₅FN₄ 128.1; m/z found 129.2 [M+H]⁺.

Step D: (E)-5-Fluoro-2-(2-(3-iodoprop-2-yn-1-ylidene)hydrazineyl)pyrimidine. To a solution of 3-iodopropiolaldehyde (240 g, 1.3 mol), and 5-fluoro-2-hydrazineylpyrimidine (162 g, 1.26 mmol), in THF (3.60 L), under an inert atmosphere of nitrogen, was added of TFA (30 g, 266 mmol) dropwise at 25° C. The reaction mixture was stirred for 2 h at room temperature. The reaction mixture was diluted with MTBE (4 L). The reaction mixture was quenched with aqueous NaHCO₃ sol'n (3 L, 7 wt %). The resulting solution was extracted with MTBE (3 L) and the organic layers were combined. The organics were washed with brine (1×3 L), separated, dried (Na₂SO₄), filtered, and concentrated under reduced pressure. The resulting residue was re-crystallized from EA:PE in the ratio of 1:2, to afford the title compound as a yellow solid (190 g, 49%). MS (ESI): mass calcd. for C₇H₄FIN₄ 290.0; m/z found 291.0 [M+H]⁺.

Step E: 5-Fluoro-4-iodo-1H-pyrazolo[3,4-b]pyridine. To a solution of (E)-5-fluoro-2-(2-(3-iodoprop-2-yn-1-ylidene)hydrazineyl)pyrimidine (190 g, 655 mmol), THF (2.90 L), and 3-pentanone (169 g, 1.96 mol), under an inert atmosphere of N₂, was added TFAA (206 g, 983 mmol) dropwise at 25° C. The reaction mixture was stirred for 5 h at 60° C. The reaction mixture was cooled to 20° C. and diluted with MTBE (3 L). The reaction mixture was quenched by the addition of aqueous NaHCO₃ sol'n (3 L, 7 wt %). The resulting solution was extracted with MTBE (2×2 L) and the organic layers were combined. The combined organics were washed with brine (1×3 L), dried (Na₂SO₄), filtered, and concentrated under reduced pressure. The resulting residue was purified by FCC (SiO₂, ethyl acetate/petroleum ether, 1:2) to afford the title compound as a yellow solid (52 g, 30%). MS (ESI): mass calcd. for C₆H₃FIN₃ 263.0; m/z found 264.0 [M+H]⁺.

Step F: 5-Fluoro-4-iodo-1-((2-(trimethylsilyl)ethoxy)methyl)-1H-pyrazolo[3,4-b]pyridine. To a cooled (0° C.) solution of 5-fluoro-4-iodo-1H-pyrazolo[3,4-b]pyridine (52 g, 198 mmol), and Cs₂CO₃ (129 g, 395 mmol), in DMF (350 mL), under an inert atmosphere of N₂, was added SEM-Cl (43 g, 257 mmol) dropwise. The reaction mixture was stirred for 2 h at 0 to 5° C. The reaction mixture was quenched with water/ice (300 mL). The resulting solution was extracted with ethyl acetate (3×400 mL) and the organic layers were combined. The combined organic layers were washed with brine (2×300 mL), dried (Na₂SO₄), filtered, and concentrated under reduced pressure. The resulting residue was purified by FCC (SiO₂, ethyl acetate/petroleum ether, 1:15) to afford the title compound as a white solid (25 g, 33%). MS (ESI): mass calcd. for C₁₂H₁₇FIN₃OSi 393.0; m/z found 394.1 [M+H]⁺. ¹H NMR (300 MHz, CDCl₃): δ 8.33-8.33 (d, J=0.9 Hz, 1H), 7.96 (s, 1H), 5.87-5.83 (d, J=14.1 Hz, 2H), 3.71-3.66 (m, 2H), 1.00-0.94 (m, 2H), −0.002 (s, 9H).

### Intermediate 205: 5-Fluoro-4-iodo-6-methyl-1-((2-(trimethylsilyl)ethoxy)methyl)-1H-pyrazolo[3,4-b]pyridine

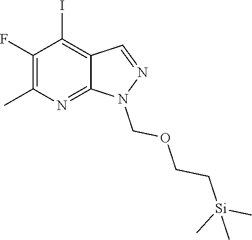 [SMILES: Cc1nc2c(cnn2COCC[Si](C)(C)C)c(I)c1F]

Step A. 2-Chloro-5-fluoro-4-methylpyrimidine. To a solution of 2,4-dichloro-5-fluoropyrimidine (600 g, 3.59 mol) in THF (6 L) under N₂, was added Fe(acac)₃ (63.5 g, 180 mmol) at 0° C. Chloromethylmagnesium (1.45 L, 4.31 mol) was added to the reaction mixture dropwise at 0° C. The resulting solution was stirred for 1 h at 0-10° C. The reaction mixture was then quenched by the addition of 1 N of HCl (2.6 L) and the resulting solution was extracted with MTBE (2×5 L). The combined organics were washed with brine (2×5 L), dried over anhydrous sodium sulfate, filtered, and concentrated under reduced pressure to yield the title compound (550 g, 94%) as a brown oil.

Step B. 5-Fluoro-2-hydrazineyl-4-methylpyrimidine. To a solution of 2-chloro-5-fluoro-4-methylpyrimidine (550 g, 3.75 mol) in EtOH (5.5 L) under N₂, was added NH₂NH₂·H₂O (1.33 kg, 22.5 mol, 85%) dropwise at 25° C. The resulting solution was stirred for 12 h at 60° C. The reaction mixture was cooled to room temperature and concentrated under reduced pressure. The resulting title compound was re-crystallized from EtOH:H₂O (1 L) in the ratio of 1:1 to yield the title compound (250 g, 47%) as an off-white solid.

Step C: (E)-5-Fluoro-2-(2-(3-iodoprop-2-yn-1-ylidene)hydrazineyl)-4-methylpyrimidine. To a solution of 3-iodopropiolaldehyde (Intermediate 204 product of Step B, 330 g, 1.83 mol), 5-fluoro-2-hydrazineyl-4-methylpyrimidine (248 g, 1.74 mol) in THF (6.6 L) at 25° C., was added TFA (20.9 g, 183 mmol) dropwise. The resulting solution was stirred for 4 h at 25° C. The resulting solution was diluted with MTBE (7 L). The pH value of the solution was adjusted to 8 with aqueous NaHCO₃ sol'n (3 L, 7 wt %) and the resulting solution was extracted with EA (2×3 L). The organics were combined and washed with brine (2×3 L) and concentrated. The title compound was re-crystallized from PE:EA in the ratio of 5:1 (3 L) to yield the title compound (286 g, 51%) as a brown solid. MS (ESI): mass calcd. for C₈H₆FIN₄ 304.0; m/z found 304.9 [M+H]⁺.

Step D. 5-Fluoro-4-iodo-6-methyl-1H-pyrazolo[3,4-b]pyridine. To a solution of (E)-5-fluoro-2-(2-(3-iodoprop-2-yn-1-ylidene)hydrazineyl)-4-methylpyrimidine (286 g, 941 mmol) and pentan-3-one (243 g, 2.82 mol) in THF (5.6 L) under N₂, was added TFAA (296 g, 1.41 mol) dropwise at 10° C. The resulting solution was stirred for 12 h at 60° C. The reaction mixture was then cooled to 20° C. and diluted with MTBE (3 L). The pH value of the solution was adjusted to 8 with aqueous NaHCO₃ sol'n (5 L, 8 wt %) and the resulting solution was extracted with ethyl acetate (2×3 L). The combined organics were then washed with brine (2×2 L), dried with Na₂SO₄, filtered, and concentrated under reduced pressure. The title compound was re-crystallized from PE:EA in the ratio of 5:1 (3 L) to yield the title compound (170 g, 20%) as a black solid. MS (ESI): mass calcd. for C₇H₅FIN₃ 277.0; m/z found 278.0 [M+H]⁺.

Step E. 5-Fluoro-4-iodo-6-methyl-1-((2-(trimethylsilyl)ethoxy)methyl)-1H-pyrazolo[3,4-b]pyridine. T a solution of 5-fluoro-4-iodo-6-methyl-1H-pyrazolo[3,4-b]pyridine (170 g, 613 mmol) in DMF (1.7 L) under N₂, was added Cs₂CO₃ (400 g, 1.23 mol) at 25° C. To the reaction mixture was added [2-(chloromethoxy)ethyl]trimethylsilane (102 g, 614 mmol) dropwise below 10° C. The resulting solution was then stirred for 4 h at 25° C. The reaction was quenched by the addition of water/ice (2 L) and the resulting solution was diluted with MTBE (2 L). The layers were separated and the aqueous was extracted with EA (2×2 L). The combined organics were washed with brine (2×2 L) and concentrated. The resulting residue was purified by silica gel column chromatography (eluting with ethyl acetate/petroleum ether (1:20)) The desired fractions were collected and the resulting product was purified by Flash-Prep-HPLC (ACN:H₂O (0.1% TFA)=4:1] to yield the title compound (25.1 g, 33%) as a light yellow solid. MS (ESI): mass calcd. for C₁₃H₁₉FIN₃OSi 407.0; m/z found 408.1 [M+H]⁺. ¹H NMR (300 MHz, CDCl₃) δ: 7.85 (s, 1H), 5.82 (s, 2H), 3.73-3.62 (m, 2H), 2.68 (d, J=3.6 Hz, 3H), 1.02-0.90 (m, 2H), −0.02 (S, 9H). ¹⁹F NMR (282 MHz, CDCl₃, ppm) δ: −117.26.

### Intermediate 206: 5-Fluoro-3-methyl-1-(tetrahydro-2H-pyran-2-yl)-4-(4,4,5,5-tetramethyl-1,3,2-dioxaborolan-2-yl)-1H-pyrazolo[3,4-b]pyridine

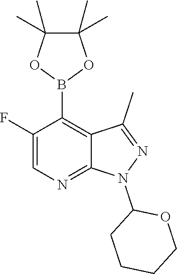 [SMILES: Cc1nn(C2CCCCO2)c2ncc(F)c(B3OC(C)(C)C(C)(C)O3)c12]

Step A: (E)-5-Fluoro-2-(2-(4-(trimethylsilyl)but-3-yn-2-ylidene)hydrazineyl)pyrimidine. Into a 10-L 4-necked round-bottom flask, was placed 5-fluoro-2-hydrazinylpyrimidine (Intermediate 204 product from Step C, 250 g, 1.95 mol) and 4-(trimethylsilyl)but-3-yn-2-one (315 g, 2.22 mol, 1.15) in THF (5 L) at 25° C. Then TFA (44.5 g, 390 mmol) was added dropwise. The resulting solution was stirred for 4 h at 25° C. The resulting solution was then diluted with MTBE (7 L). The pH value of the solution was adjusted to 8 with aqueous NaHCO₃ sol'n (3 L, 7 wt %) and the resulting solution was extracted with EA (2×3 L). The combined organics were then washed with brine (2×3 L) and concentrated. The title compound was re-crystallized from PE:EA in the ratio of 5:1 (3 L) to afford the title compound (460 g, 89%) as a brown solid. MS (ESI): mass calcd. for C₁₁H₁₅FN₄Si 250.1; m/z found 251.1 [M+H]⁺.

Step B. 5-Fluoro-3-methyl-4-(trimethylsilyl)-1H-pyrazolo[3,4-b]pyridine. Into a 10-L 4-necked round-bottom flask purged and maintained with an inert atmosphere of nitrogen, was placed a solution of (E)-5-fluoro-2-(2-(4-(trimethylsilyl)but-3-yn-2-ylidene)hydrazineyl)pyrimidine (460 g, 1.75 mol), pentan-3-one (451 g, 5.24 mol) in THF (4.6 L). This was followed by the addition of TFAA (550 g, 2.62 mmol, 1.50) dropwise at 10° C. The resulting solution was stirred for 12 h at 60° C. The reaction mixture was then cooled to 20° C. and diluted with MTBE (3 L). The pH value of the solution was adjusted to 8 with aqueous NaHCO₃ sol'n (5 L, 8 wt %) and the resulting solution was extracted with ethyl acetate (2×3 L). The combined organics were washed with brine (2×2 L), dried with Na₂SO₄, filtered, and concentrated. The title compound was re-crystallized from PE:EA in the ratio of 5:1 (3 L) to yield the title compound (350 g, 81%) as a yellow solid. MS (ESI): mass calcd. for C₁₀H₁₄FN₃Si 223.1; m/z found 224.1 [M+H]⁺.

Step C. 5-Fluoro-3-methyl-1H-pyrazolo[3,4-b]pyridine. Into a 3-L 4-necked round-bottom flask purged and maintained with an inert atmosphere of nitrogen, was placed a solution of 5-fluoro-3-methyl-4-(trimethylsilyl)-1H-pyrazolo[3,4-b]pyridine (350 g, 1.41 mol) in THF (1.5 L). This was followed by the addition of TBAF (184 g, 705 mmol) in 3 batches at 0° C. The resulting solution was stirred for 2 h at 25° C. The reaction was then quenched by the addition of water/ice (2 L) and the resulting mixture was extracted with ethyl acetate (2×2 L). The combined organics were then washed with brine (2×1 L), dried over anhydrous sodium sulfate, filtered, and concentrated. The residue was purified by silica gel column chromatography (eluting with ethyl acetate/petroleum ether (1:1)) to yield the title compound (148 g, 69%) as a white solid. MS (ESI): mass calcd. for C₇H₆FN₃ 151.1; m/z found 152.1 [M+H]⁺.

Step D. 5-Fluoro-3-methyl-1H-pyrazolo[3,4-b]pyridine 7-oxide. Into a 3-L 4-necked round-bottom flask purged and maintained with an inert atmosphere of nitrogen, was placed a solution of 5-fluoro-3-methyl-1H-pyrazolo[3,4-b]pyridine (148 g, 979 mmol) in MTBE (1.5 L). This was followed by the addition of m-CPBA (338 g, 1.96 mol) in 3 batches at 25° C. The resulting solution was stirred overnight at 25° C. The solids were collected by filtration, washed with MTBE (2×500 mL), and dried at 40° C. for 1 h to yield the title compound (131 g, 80%) as a white solid. MS (ESI): mass calcd. for C₇H₆FN₃O 167.1; m/z found 168.2 [M+H]⁺.

Step E. 4-Bromo-5-fluoro-3-methyl-1H-pyrazolo[3,4-b]pyridine. Into a 3-L 4-necked round-bottom flask purged and maintained with an inert atmosphere of nitrogen, was placed a solution of 5-fluoro-3-methyl-1H-pyrazolo[3,4-b]pyridine 7-oxide (131 g, 784 mmol) in DMF (1.5 L). This was followed by the addition of phosphoryl bromide (449 g, 1.57 mol) in 3 batches at 10° C. The resulting solution was stirred for 5 h at 25° C. The reaction was then quenched by the addition of water/ice (5 L). The pH value of the solution was adjusted to 8 with NaHCO₃(750 g). The resulting solution was extracted with ethyl acetate (3×2 L). The combined organics were washed with brine (3×2 L), dried over anhydrous sodium sulfate, filtered, and concentrated. The residue was purified by silica gel column chromatography (eluting with ethyl acetate/petroleum ether (1:1)) to yield the title compound (40 g, 21%) as a white solid. MS (ESI): mass calcd. for C₇H₅BrFN₃ 229.0; m/z found 230.0 [M+H]⁺.

Step F. 4-Bromo-5-fluoro-3-methyl-1-(tetrahydro-2H-pyran-2-yl)-1H-pyrazolo[3,4-b]pyridine. Into a 1-L 4-necked round-bottom flask purged and maintained with an inert atmosphere of nitrogen, was placed a solution of 4-bromo-5-fluoro-3-methyl-1H-pyrazolo[3,4-b]pyridine (40 g, 165 mmol), (+/−)-camphor-10-sulfonic acid (5.8 g, 25 mmol) in THF (400 mL). This was followed by the addition of dihydropyran (69.5 g, 826 mmol) dropwise at 25° C. The resulting solution was stirred for 3 h at 25° C. The reaction was then quenched by the addition of water/ice (500 mL). The pH value of the solution was adjusted to 8 with aqueous NaHCO₃ sol'n (1 L, 8 wt %) and the resulting solution was extracted with ethyl acetate (2×2 L). The combined organics were washed with brine (2×2 L), dried with Na₂SO₄, filtered, and concentrated. The title compound was purified by re-crystallization from hexane (500 mL) to afford the title compound (25 g, 48%) of as a white solid. MS (ESI): mass calcd. for C₁₂H₁₃BrFN₃O 313.0; m/z found 314.0.0 [M+H]⁺.

Step G. 5-Bluoro-3-methyl-1-(tetrahydro-2H-pyran-2-yl)-4-(4,4,5,5-tetramethyl-1,3,2-dioxaborolan-2-yl)-1H-pyrazolo[3,4-b]pyridine. KOAc (936 mg, 9.55 mmol) was added to a mixture of 4-bromo-5-fluoro-3-methyl-1-(tetrahydro-2H-pyran-2-yl)-1H-pyrazolo[3,4-b]pyridine (1.0 g, 3.2 mmol) and 4,4,5,5-tetramethyl-2-(1,4-dioxaspiro[4.5]dec-7-en-8-yl)-1,3,2-dioxaborolane (970 mg, 3.82 mmol) in 1,4-dioxane (20 mL). The mixture was sparged with Ar for 5 minutes and then treated with Pd(dppf)Cl₂ (130 mg, 0.159 mmol). The mixture was sparged with Ar for another 5 minutes and then heated at 90° C. for 16 hours after which LCMS analysis showed the reaction was complete. The reaction mixture was filtered and purified by FCC (ethyl acetate:petroleum ether=1:10 to 1:3) to yield the title compound (1 g, 61% yield, 70% purity) as a white solid. MS (ESI): mass calcd. for C₁₈H₂₅BFN₃O₃ 361.2; m/z found 362.1 [M+H]⁺.

### Intermediate 207: 4-Chloro-6-(difluoromethyl)-3-methyl-1-((2-(trimethylsilyl)ethoxy)methyl)-1H-pyrazolo[3,4-b]pyridine

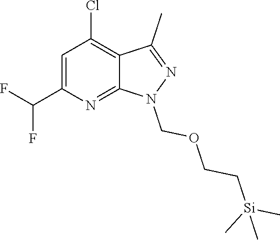 [SMILES: Cc1nn(COCC[Si](C)(C)C)c2nc(C(F)F)cc(Cl)c12]

Step A. 4-Chloro-6-(difluoromethyl)-3-methyl-1H-pyrazolo[3,4-b]pyridine. To a mixture of 4-chloro-3-methyl-1H-pyrazolo[3,4-b]pyridine (500 mg, 2.98 mmol), sodium difluoromethanesulfinate (2.06 g, 14.9 mmol), and water (5 mL) was added TFA until a pH of 7 was reached. Additional TFA (0.31 mL, 4.2 mmol) was then added followed by DCM (15 mL) and dropwise addition of tert-butyl hydroperoxide (2.47 mL, 15.5 mmol). The mixture was stirred for 1 h at room-temperature before diluting with sat. NaHCO₃ sol'n (25 mL) and DCM (25 mL). The reaction mixture was stirred for 10 min, then the organic phase was separated and the aqueous phase was extracted with methylene chloride (20 mL×3). The combined organic layers were dried over Na₂SO₄, filtered, and concentrated under reduced pressure. The resulting residue was purified by FCC (SiO₂, from petroleum ether:ethyl acetate=10:1 to 1:1) to give the title compound. ¹H NMR (400 MHz, CDCl₃) δ7.45 (s, 1H), 6.88-6.52 (m, 1H), 2.85-2.71 (m, 3H).

Step B. 4-Chloro-6-(difluoromethyl)-3-methyl-1-((2-(trimethylsilyl)ethoxy)methyl)-1H-pyrazolo[3,4-b]pyridine. Sodium hydride in mineral oil (83 mg, 60% purity, 2.1 mmol) was added in portions to a 0° C. (ice/water) solution consisting of 4-chloro-6-(difluoromethyl)-3-methyl-1H-pyrazolo[3,4-b]pyridine (300 mg, 1.38 mmol) in THF (10 mL). After 15 mins SEMCl (317 uL, 1.79 mmol) was added at 0° C. and the reaction mixture was stirred for 16 hours at room-temperature. The reaction mixture was quenched with sat. NH₄Cl (10 mL) and extracted with ethyl acetate (10 mL×2). The combined organic extracts were washed with brine (10 mL), dried over anhydrous Na₂SO₄, filtered, and concentrated. Purification by FCC (eluent: petroleum ether:ethyl acetate=1:0 to 10:1) afforded the title compound (180 mg, 27%) as a yellow oil. MS (ESI): mass calcd. for C₁₄H₂₀ClF₂N₃OSi 347.1; m/z found 348.0 [M+H]⁺.

### Intermediate 208: 4-Bromo-5-fluoro-6-methyl-1-((2-(trimethylsilyl)ethoxy)methyl)-1H-pyrazolo[3,4-b]pyridine

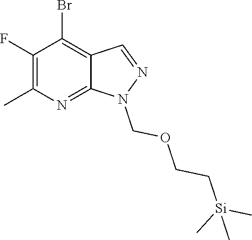 [SMILES: Cc1nc2c(cnn2COCC[Si](C)(C)C)c(Br)c1F]

Step A: (Z)-5-Fluoro-4-methyl-2-(2-(4-(trimethylsilyl)but-3-yn-2-ylidene)hydrazineyl)pyrimidine. A solution of 4-(trimethylsilyl)but-3-yn-2-one (417.90 g, 2979.53 mmol), 5-fluoro-2-hydrazineyl-4-methylpyrimidine (Intermediate 205 product from Step B, 385.00 g, 2708.65 mmol), and TFA (61.77 g, 541.731 mmol) in THF (6.00 L), under an inert atmosphere of N₂, was stirred for 2 h at 25° C. The reaction mixture was diluted with EA (5 L), and quenched with aqueous NaHCO₃ sol'n (5 L, 7 wt %). The resulting solution was extracted with ethyl acetate (2×5 L). The combined organic layers were separated, dried (Na₂SO₄), filtered, and concentrated under vacuum to afford the title compound (688 g, 96%) as a yellow solid. MS (ESI): mass calcd. for C₁₂H₁₇FN₄Si 264.1; m/z found 265.1 [M+H]⁺.

Step B: 5-Fluoro-3,6-dimethyl-4-(trimethylsilyl)-1H-pyrazolo[3,4-b]pyridine. To a solution (20° C.) of (Z)-5-fluoro-4-methyl-2-(2-(4-(trimethylsilyl)but-3-yn-2-ylidene)hydrazineyl)pyrimidine (688.00 g, 2602.32 mmol), and 3-pentanone (672 g, 7814 mmol), in THF (6.0 L) was added TFAA (819.85 g, 3903.48 mmol, 1.50) dropwise. The reaction mixture was stirred for 2-3 h at 60° C. The reaction mixture was cooled to 25° C. and diluted with MTBE (8 L). The reaction mixture was quenched with ice/aqueous NaHCO₃ sol'n (6 L, 7 wt %). The resulting solution was extracted with MTBE (2×5 L), and the combined organic layers were washed with brine (2×4 L). The organic layers were separated, dried (Na₂SO₄), filtered, and concentrated under vacuum. The resulting solids were diluted with 1 L (3 V) of PE. The resulting solids were collected by filtration and re-crystallized from EA in the ratio of 500 mL (2V) to afford the title compound (200 g, 33%) as a light yellow solid. MS (ESI): mass calcd. for C₁₁H₁₆FN₃Si 237.1; m/z found 238.2 [M+H]⁺.

Step C: 5-Fluoro-3,6-dimethyl-1H-pyrazolo[3,4-b]pyridine. To a solution (20° C.) of 5-fluoro-3,6-dimethyl-4-(trimethylsilyl)-1H-pyrazolo[3,4-b]pyridine (200.00 g, 842.62 mmol), in THF (2.00 L), under an inert atmosphere of N₂, was added TBAF·3H₂O (133.00 g, 422.222 mmol), in portions. The reaction mixture was stirred for 1 h at 25° C. The reaction mixture was diluted with MTBE (3 L). The resulting solution was quenched with water/ice (2 L) and extracted with MTBE (2 L). The combined organics were washed with brine (2×2 L), dried over anhydrous sodium sulfate, filtered, and concentrated under reduced pressure to afford the title compound (125 g, 90%) as a light yellow solid. MS (ESI): mass calcd. for C₈H₈FN₃ 165.1; m/z found 166.2 [M+H]⁺.

Step D: 5-Fluoro-3,6-dimethyl-1H-pyrazolo[3,4-b]pyridine 7-oxide. To a solution (20° C.) of 5-fluoro-3,6-dimethyl-1H-pyrazolo[3,4-b]pyridine (125.00 g, 756.79 mmol), in MTBE (2.50 L), under an inert atmosphere of N₂, was added m-CPBA (261.19 g, 1513.56 mmol) portion-wise. The reaction mixture was stirred for 15 h at 25° C. The resulting solids were collected by filtration and washed with MTBE (300 mL) to afford the title compound (110 g, 80%) as a white solid. MS (ESI): mass calcd. for C₈H₈FN₃O 181.1; m/z found 182.2 [M+H]⁺.

Step E: 4-Bromo-5-fluoro-3,6-dimethyl-1H-pyrazolo[3,4-b]pyridine. To a cooled solution (0° C.) of 5-fluoro-3,6-dimethyl-1H-pyrazolo[3,4-b]pyridine 7-oxide (110.00 g, 607.16 mmol), in DMF (1.10 L), under an inert atmosphere of N₂, was added POBr₃ (226.28 g, 789.298 mmol, 1.30 equiv), in portions. The reaction mixture was stirred for 1 h at 0 to 5° C. The reaction mixture was quenched with water/ice (500 mL). The pH value of the solution was adjusted to 8 with ice/aqueous NaHCO₃ sol'n (7 wt %). The resulting solution was extracted with ethyl acetate (3×1.5 L) and the organic layers were combined. The combined organic layers were washed with brine (2×1.5 L), separated, dried over anhydrous sodium sulfate, filtered, and concentrated under reduced pressure. The resulting residue was re-crystallized from PE 100 mL (2 V). The solids were collected by filtration to afford the title compound (51 g, 34%) as a light yellow solid. MS (ESI): mass calcd. for C₈H₇BrFN₃ 243.0; m/z found 244.0 [M+H]⁺.

Step F: 4-Bromo-5-fluoro-6-methyl-1-((2-(trimethylsilyl)ethoxy)methyl)-1H-pyrazolo[3,4-b]pyridine. To a cooled solution (0° C.) of 4-bromo-5-fluoro-3,6-dimethyl-1H-pyrazolo[3,4-b]pyridine (40.00 g, 163.88 mmol), and Cs₂CO₃ (106.80 g, 327.77 mmol), in DMF (400.00 mL), under an inert atmosphere of N₂, was added SEM-Cl (35.52 g, 213.05 mmol) dropwise. The reaction mixture was stirred for 3 h at 0° C. The reaction mixture was quenched with water/ice (500 mL). The resulting solution was extracted with ethyl acetate (3×500 mL) and the organic layers were combined. The combined organic layers were washed with brine (2×300 mL), separated, dried over anhydrous sodium sulfate, filtered, and concentrated under reduced pressure. The resulting residue was purified by FCC (SiO₂, ethyl acetate/petroleum ether (1:15)) to afford the title compound (26.1 g, 43%) as a white solid. MS (ESI): mass calcd. for C₁₄H₂₁BrFN₃OSi 373.1; m/z found 374.1 [M+H]⁺. ¹H (300 MHz, CDCl₃): δ 5.75 (s, 2H), 3.71-3.60 (m, 2H), 2.75-2.62 (m, 6H), 1.02-0.90 (m, 2H), −0.02 (s, 9H).

### Intermediate 209: 4-Bromo-5-fluoro-3,6-dimethyl-1-((2-(trimethylsilyl)ethoxy)methyl)-1H-pyrazolo[3,4-b]pyridine

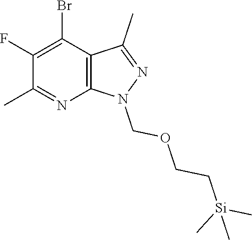 [SMILES: Cc1nc2c(c(C)nn2COCC[Si](C)(C)C)c(Br)c1F]

Step A: (Z)-5-Fluoro-4-methyl-2-(2-(4-(trimethylsilyl)but-3-yn-2-ylidene)hydrazineyl)pyrimidine. Under an atmosphere of N₂, a mixture of 4-(trimethylsilyl)but-3-yn-2-one (380 g, 2.71 mol), 5-fluoro-2-hydrazineyl-4-methylpyrimidine (Intermediate 205, Step B, 350 g, 2.46 mol), and TFA (56 g, 49 mol) in THF (1 L) was stirred for 2 h at 25° C. after which LCMS showed the reaction was complete. The reaction mixture was then diluted with EtOAc (500 mL) and quenched with aqueous NaHCO₃ sol'n (1 L, 7 wt %). The layers were separated and the aqueous was extracted with EtOAc (2×500 mL). The combined organics were dried with MgSO₄, filtered, and concentrated to afford the title compound (895 g, 60% yield, 87% purity) as a brown oil. MS (ESI): mass calcd. for C₁₂H₁₇FN₄Si 264.1; m/z found 265.2 [M+H]⁺. ¹H (400 MHz, CDCl₃): δ 9.00 (s, 1H), 8.19 (d, J=1.20 Hz, 1H), 2.44 (d, J=2.40 Hz, 3H), 2.20 (s, 3H), 0.29 (s, 9H).

Step B: 5-Fluoro-3,6-dimethyl-4-(trimethylsilyl)-1H-pyrazolo[3,4-b]pyridine. Under an atmosphere of N₂, TFAA (527 g, 5.02 mol, 532 mL) was slowly added to a mixture of (Z)-5-fluoro-4-methyl-2-(2-(4-(trimethylsilyl)but-3-yn-2-ylidene)hydrazineyl)pyrimidine (442 g, 1.67 mol), and 3-pentanone (432 g, 5.02 mol) in THF (2.25 L). The reaction mixture was then stirred for 3 h at 60° C. after which LCMS analysis showed the reaction was complete. The reaction mixture was then quenched with ice/NaHCO₃ sol'n (6 L, 7 wt %) and the resulting solution was extracted with MTBE (5×3 L). The combined organics were then washed with brine (3 L), dried with Na₂SO₄, filtered, and concentrated. The title compound was re-crystallized from EtOAc (300 mL, 2V) to obtain the title compound (226 g) as a light yellow solid. The residue from the recrystallization was further purified by column chromatography (SiO₂, Petroleum ether/Ethyl acetate=0% to 100%) to yield additional title compound (10.0 g) as a light yellow solid for combined recovery of 236 g and 30% overall yield. MS (ESI): mass calcd. for C₁₁H₁₆FN₃Si 237.1; m/z found 238.2 [M+H]⁺. ¹H (400 MHz, CDCl₃): δ 10.05-8.48 (m, 1H), 2.76-2.61 (m, 6H), 0.53 (d, J=2.40 Hz, 9H).

Step C: 5-Fluoro-3,6-dimethyl-1H-pyrazolo[3,4-b]pyridine. To a mixture of 5-fluoro-3,6-dimethyl-4-(trimethylsilyl)-1H-pyrazolo[3,4-b]pyridine (226 g, 952 mmol), in THF (2.25 L), under an inert atmosphere of N₂, was added TBAF (1M, 476 ml, 476 mmol), in one portion. The reaction mixture was stirred for 2 h at 25° C. after which LCMS analysis showed the reaction was complete. The reaction mixture was diluted with MTBE (2 L) and the resulting solution was quenched with water (2 L). The layers were separated and the aqueous was extracted with MTBE (2×2 L). The combined organics were washed with brine (2×2 L), dried over anhydrous sodium sulfate, filtered, and concentrated under reduced pressure to afford the title compound (165 g, crude) as a light yellow solid. MS (ESI): mass calcd. for C₈H₈FN₃ 165.1; m/z found 166.2 [M+H]⁺. ¹H (400 MHz, CDCl₃): δ 11.96 (br s, 1H), 7.61 (d, J=8.80 Hz, 1H), 2.71 (d, J=3.20 Hz, 3H), 2.57 (s, 3H).

Step D: 5-Fluoro-3,6-dimethyl-1H-pyrazolo[3,4-b]pyridine 7-oxide. To a mixture of 5-fluoro-3,6-dimethyl-1H-pyrazolo[3,4-b]pyridine (163 g, 987 mmol), in MTBE (3.26 L), under an inert atmosphere of N₂, was added m-CPBA (401 g, 1.97 mmol, 80% purity). The reaction mixture was stirred for 15 h at 25° C. after which LCMS analysis showed the reaction was complete. The resulting solids were collected by filtration and washed with MTBE (300 mL) to afford the title compound (156 g, 87% yield, 99% purity) as a white solid. MS (ESI): mass calcd. for C₈H₈FN₃O 181.1; m/z found 182.2 [M+H]⁺. ¹H (400 MHz, CDCl₃): δ 14.60-12.92 (m, 1H), 7.44 (d, J=6.80 Hz, 1H), 2.75 (d, J=2.80 Hz, 3H), 2.55 (s, 3H).

Step E: 4-Bromo-5-fluoro-3,6-dimethyl-1H-pyrazolo[3,4-b]pyridine. To a cooled solution (0° C.) of 5-fluoro-3,6-dimethyl-1H-pyrazolo[3,4-b]pyridine 7-oxide (88.4 g, 488 mmol), in DMF (900 mL), under an inert atmosphere of N₂, was added POBr₃ (285 g, 976 mmol, 101 mL). The reaction mixture was stirred for 1 h at 25° C. after which LCMS analysis showed the reaction was complete. The reaction mixture was then combined with an earlier batch and quenched with water/ice (5 L). The pH value of the solution was adjusted to 8 with ice/NaHCO₃ sol'n (7 wt %). The resulting solution was extracted with ethyl acetate (3×5 L) and the organic layers were combined. The combined organic layers were washed with brine (2×5 L), dried over anhydrous sodium sulfate, filtered, and concentrated under reduced pressure. The title compound was triturated with petroleum (200 mL). MS (ESI): mass calcd. for C₈H₇BrFN₃ 243.0; m/z found 246.1 M+2+H]⁺.

Step F: 4-Bromo-5-fluoro-3,6-dimethyl-1-((2-(trimethylsilyl)ethoxy)methyl)-1H-pyrazolo[3,4-b]pyridine. To a cooled solution (0° C.) of 4-bromo-5-fluoro-3,6-dimethyl-1H-pyrazolo[3,4-b]pyridine (36.0 g, 147 mmol), and Cs₂CO₃ (96.1 g, 295 mmol), in DMF (200.00 mL), under an inert atmosphere of N₂, was added SEM-Cl (49.2 g, 295 mmol). The reaction mixture was stirred for 3 h at 0° C. The reaction mixture was quenched with water/ice (1 L) and the resulting solution was extracted with EtOAc (3×1 L) and the organic layers were combined. The combined organic layers were washed with brine (2×1 L), dried over anhydrous sodium sulfate, filtered, and concentrated under reduced pressure. The resulting residue was combined with earlier batches and purified by FCC (SiO₂, Petroleum ether/Ethyl acetate=0% to 4%) to provide impure material. The impure material was triturated with petroleum ether (30 mL, 0.8V) for 20 min at 25° C. and then filtered and the cake collected. The trituration process was carried out four times to yield the title compound (26.8 g) as a white solid. The filtrate from the trituration was concentrated and the impure material was triturated with petroleum ether (5 mL, 0.8V) for 40 min at 25° C. and then filtered and the cake collected. The trituration process was carried out four times to yield additional title compound (4.1 g) as a white solid. Combined title compound (30.9 g). MS (ESI): mass calcd. for C₁₄H₂₁BrFN₃OSi 373.1; m/z found 374.2 [M+H]⁺. ¹H (400 MHz, CDCl₃): δ 5.73 (s, 2H), 3.66-3.61 (m, 2H), 2.71 (s, 3H), 2.64 (d, J=3.60 Hz, 3H), 0.97-0.93 (m, 2H), −0.04 (s, 9H).

### Intermediate 210: 4-Bromo-5-fluoro-1-((2-(trimethylsilyl)ethoxy)methyl)-1H-pyrrolo[2,3-b]pyridine

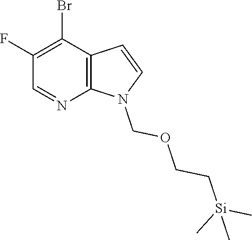 [SMILES: C[Si](C)(C)CCOCn1ccc2c(Br)c(F)cnc21]

The title compound was prepared in a manner analogous to Intermediate 19, except using 4-bromo-5-fluoro-1H-pyrrolo[2,3-b]pyridine instead of 7-chloro-1H-pyrazolo[4,3-b]pyridine. MS (ESI): mass calcd. for C₁₃H₁₈BrFN₂OSi 344.0; m/z found 345.1 [M+H−106]⁺.

### Intermediate 211: 7-Bromo-1-((2-(trimethylsilyl)ethoxy)methyl)-1H-pyrazolo[4,3-b]pyridine

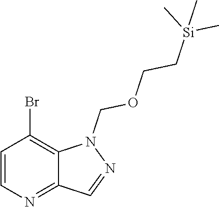 [SMILES: C[Si](C)(C)CCOCn1ncc2nccc(Br)c21]

The title compound was prepared in a manner analogous to 7-chloro-1-((2-(trimethylsilyl)ethoxy)methyl)-1H-pyrazolo[4,3-b]pyridine (Intermediate 19), except using 7-bromo-1H-pyrazolo[4,3-b]pyridine instead of 7-chloro-1H-pyrazolo[4,3-b]pyridine. MS (ESI): mass calcd. for C₁₂H₁₈BrN₃OSi 327.04; m/z found 327.8 [M+H]⁺.

### Intermediate 212: 4-Chloro-6-methyl-1-((2-(trimethylsilyl)ethoxy)methyl)-1H-pyrazolo[3,4-d]pyrimidine

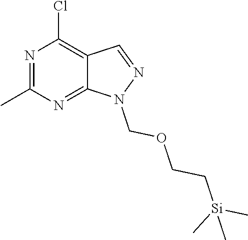

Sodium hydride in mineral oil (300 mg, 60% purity, 7.5 mmol) was added in portions to a 0° C. solution of 4-chloro-6-methyl-1H-pyrazolo[3,4-d]pyrimidine (1.0 g, 5.9 mmol) in THF (30 mL). SEM-Cl (1.40 mL, 7.91 mmol) was added to the reaction mixture at 0° C. The resultant mixture was stirred for 2 hours. The reaction mixture was gradually warmed to room-temperature. The reaction mixture was poured into sat. aq. NH₄Cl (30 mL) and extracted with ethyl acetate (30 mL×3). The combined organic extracts were washed with brine (30 mL), dried over anhydrous Na₂SO₄, filtered, and concentrated to dryness under reduced pressure. The resulting residue was purified by FCC (SiO₂, 0-5% EtOAc/petroleum ether) to afford the title compound (650 mg, 35%) as a white solid. MS (ESI): mass calcd. for C₁₂H₁₉ClN₄OSi 298.1; m/z found 298.9 [M+H]⁺.

### Intermediate 213: 5-Fluoro-4-iodo-1-(tetrahydro-2H-pyran-2-yl)-1H-pyrazolo[3,4-b]pyridine

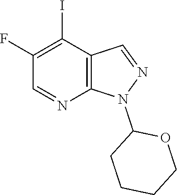 [SMILES: Fc1cnc2c(cnn2C2CCCCO2)c1I]

To a solution of 5-fluoro-4-iodo-1H-pyrazolo[3,4-b]pyridine (Intermediate 204 product from Step E, 74.6 g, 283 mmol, 1.00 equiv), THF (746 mL), 3,4-dihydro-2H-pyran (120 g, 1428 mol, 5.00 equiv) under N₂, was added TsOH (10.0 g, 42.45 mmol, 0.15 equiv). The reaction mixture was stirred for 2 h at 25° C. The reaction mixture was concentrated under reduced pressure. The resulting residue was purified by FCC (SiO₂, ethyl acetate/petroleum ether (1:15)) to afford the title compound as a white solid (46.2 g, 51%). MS (ESI): mass calcd. for C₁₁H₁₁FIN₃O 347.0; m/z found 348.0 [M+H]⁺. ¹H NMR (300 MHz, DMSO-d₆): δ 8.50 (d, J=1.1 Hz, 1H), 8.10 (s, 1H), 5.98 (dd, J=10.3, 2.5 Hz, 1H), 3.97-3.90 (m, 1H), 3.74-3.65 (m, 1H), 2.56-2.37 (m, 1H), 2.11-1.99 (m, 1H), 2.04-1.71 (m, 2H), 1.62-1.53 (m, 2H).

### Intermediate 214: 5-Fluoro-4-iodo-3-methyl-1-(tetrahydro-2H-pyran-2-yl)-1H-pyrazolo[3,4-b]pyridine

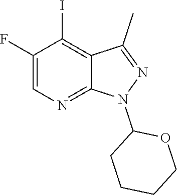 [SMILES: Cc1nn(C2CCCCO2)c2ncc(F)c(I)c12]

Step A. 5-Fluoro-4-iodo-3-methyl-1H-pyrazolo[3,4-b]pyridine. To a solution of 4-iodobut-3-yn-2-one (Intermediate 202 product from Step A, 4.8 g, 23 mmol) and 5-fluoro-2-hydrazineylpyrimidine (Intermediate 204 product from Step C, 3.3 g, 25 mmol) was added THF (103 mL) and TFA (0.089 mL). The reaction mixture was stirred for 1 hr, became cloudy then became a thick slurry. TFAA (9.7 mL, 69 mmol) was added, followed by pentanone (7.3 mL, 69 mmol) and the reaction mixture was heated to 60° C. for 1 hr. The reaction mixture was cooled, diluted with EtOAc (600 mL) and washed with sat aq Na₂CO₃ (200 mL). The organic layer was separated, dried over MgSO₄, filtered, dry loaded onto Celite®, and purified by FCC (SiO₂, Hex/EA 0-60%) to give a dark brown solid. The solids were slurred in MeCN (10 mL), filtered, washed with additional MeCN (4 mL), and dried on high-vac to yield the title compound as a tan colored solid (5.2 g, 74%). MS (ESI): mass calcd. for C₈H₆FIN₄ 277.0; m/z found 278.0 [M+H]⁺. ¹H NMR (500 MHz, DMSO-d₆) δ 8.35 (d, J=1.1 Hz, 1H), 2.62 (s, 3H).

Step B. 5-Fluoro-4-iodo-3-methyl-1-(tetrahydro-2H-pyran-2-yl)-1H-pyrazolo[3,4-b]pyridine. To a solution of 5-fluoro-4-iodo-3-methyl-1H-pyrazolo[3,4-b]pyridine (200 mg, 0.7 mmol) and 3,4-dihydro-2 h-pyran (0.14 g, 1.6 mmol) in THF (3.6 mL) was added p-toluenesulfonic acid monohydrate (34 mg, 0.18 mmol, 0.25 eq.). The resulting mixture was stirred at room temperature overnight after which it was diluted with EA/water and the aqueous layer extracted with EA (2×20 mL). The organics were concentrated to produce a yellow solid which was purified by silica gel chromatography (0-20% EA/hex,) to afford the title compound as a white solid (252 mg, 97%). MS (ESI): mass calcd. for C₁₂H₁₃FIN₃O 361.0; m/z found 362.0 [M+H]⁺. ¹H NMR (500 MHz, CDCl₃): δ 8.20 (d, J=0.9 Hz, 1H), 5.99 (dd, J=10.7, 2.5 Hz, 1H), 4.15-4.08 (m, 1H), 3.81-3.76 (m, 1H), 2.72 (s, 3H), 2.64-2.56 (tdd, J=12.9, 10.7, 4.3 Hz, 1H), 2.19-2.10 (m, 1H), 1.97-1.90 (m, 1H), 1.84-1.71 (m, 2H), 1.66-1.58 (m, 1H).

### Intermediate 215: 5-Fluoro-4-iodo-6-methyl-1-(tetrahydro-2H-pyran-2-yl)-1H-pyrazolo[3,4-b]pyridine

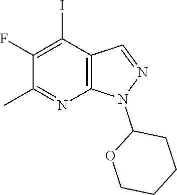 [SMILES: Cc1nc2c(cnn2C2CCCCO2)c(I)c1F]

Into a 2-L 4-necked round-bottom flask purged and maintained with an inert atmosphere of nitrogen, was placed a solution of 5-fluoro-4-iodo-6-methyl-1H-pyrazolo[3,4-b]pyridine (Intermediate 205, product from Step D, 55 g, 198.52 mmol), DL-Camphor sulfonic acid (6.92 g, 29.78 mmol) in THF (550 mL). This was followed by the addition of dihydropyran (83.5 g, 992.67 mmol, 5.00 equiv) dropwise at 25° C. The resulting solution was stirred for 3 hours at 25° C. The reaction was then quenched by the addition of 500 mL of water/ice. The pH value of the solution was adjusted to 8 with NaHCO₃(1 L, 8%). The resulting solution was extracted with 2×2 L of ethyl acetate and washed with 2×2 L of brine, then dried by Na₂SO₄, filtered, and concentrated under reduced pressure. Purification (by Prep-HPLC (ACN:H₂O (0.1% TFA)=7:3)) afforded the title compound as a white solid (50.01 g, 65%). MS (ESI): mass calcd. for C₁₂H₁₃FIN₃O 361.0; m/z found 362.0 [M+H]⁺. ¹H NMR (300 MHz, DMSO-d₆) δ 7.99 (s, 1H), 5.94 (dd, J=10.3, 2.5 Hz, 1H), 3.93 (d, J=11.5 Hz, 1H), 3.75-3.62 (m, 1H), 2.59 (d, J=3.5 Hz, 3H), 2.46-2.37 (m, 1H), 2.05-2.01 (m, 1H), 1.95-1.67 (m, 2H), 1.61-1.54 (m, 2H). ¹⁹F NMR (282 MHz, CDCl₃): δ −118.07.

### Intermediate 217: 1-(4-Methoxybenzyl)-6-methyl-4-(4,4,5,5-tetramethyl-1,3,2-dioxaborolan-2-yl)-1H-pyrazolo[3,4-b]pyridine

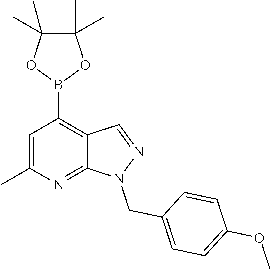

Step A: Ethyl 5-amino-1-(4-methoxybenzyl)-1H-pyrazole-4-carboxylate.

To a mixture of ethyl (E)-2-cyano-3-ethoxyacrylate (210 g, 1.24 mol) and (4-methoxybenzyl)hydrazine hydrochloride salt (258 g, 1.37 mol) in EtOH (1.20 L) was added TEA (176 g, 1.74 mol, 242 mL) at 25° C. The reaction mixture was stirred at 80° C. for 14 hours after which it was concentrated under reduced pressure to remove EtOH. Water (1.00 L) was then added to the residual solid and stirred for 2 hrs. The mixture was then filtered and the precipitate was washed with water (300 mL*3). The precipitate was then slurried in petroleum ether (1.00 L), filtered, and the cake washed with petroleum ether (300 mL*3) to afford the title compound (340 g, 1.24 mol, crude) as a white solid. The title compound was used in the next step without further purification. MS (ESI): mass calcd. for C₁₄H₁₇N₃O 275.1; m/z found 275.9 [M+H]⁺. ¹H NMR (400 MHz, CDCl₃) δ 7.67 (s, 1H), 7.14 (d, J=8.80 Hz, 2H), 6.88 (d, J=8.80 Hz, 2H), 5.10 (s, 2H), 4.84 (s, 2H), 4.26 (q, J=7.20 Hz, 2H), 3.80 (s, 3H), 1.33 (t, J=6.80 Hz, 3H).

Step B: 5-Amino-1-(4-methoxybenzyl)-1H-pyrazole-4-carboxylic acid. To a mixture of ethyl 5-amino-1-(4-methoxybenzyl)-1H-pyrazole-4-carboxylate (334 g, 1.21 mol) in EtOH (1.80 L) was added NaOH (97.1 g, 2.43 mol, 2 eq) and H₂O (300 mL) in one portion at 20° C. The reaction mixture was stirred at 80° C. for 12 hours After which the reaction mixture was cooled and concentrated under reduced pressure. The resulting residue was diluted with H₂O (2.00 L) and the pH was adjusted to pH 5-6 by addition 18% HCl (˜264 mL). The resulting mixture was stirred for 30 min, then filtered. The resulting filter cake was washed with H₂O. The filter cake solids were slurried in water twice and filtered. The resulting filter cake was washed with petroleum ether to give yellow gum. EtOH was added to the resulting gum, then concentrated under pressure to afford the title compound (265 g, 1.06 mol, 88% yield, 99% purity) as a yellow solid. MS (ESI): mass calcd. for C₁₂H₁₃N₃O 247.1; m/z found 248.2 [M+H]⁺. ¹H NMR (400 MHz DMSO-d₆) δ 7.43 (s, 1H), 7.14 (d, J=8.40 Hz, 2H), 6.88 (d, J=8.40 Hz, 2H), 6.28 (br s, 2H), 5.07 (s, 2H), 3.72 (s, 3H).

Step C: 1-(4-Methoxybenzyl)-1H-pyrazol-5-amine. 5-Amino-1-(4-methoxybenzyl)-1H-pyrazole-4-carboxylic acid (210 g, 841 mmol, 99% purity) was added in a single-necked round bottom flask and stirred at 140° C. for 2 hours under N₂. The reaction mixture was filtered and concentrated under reduced pressure to give a yellow gum. The resulting residue was triturated with petroleum ether at 20° C. for 12 hours to afford the title compound (150 g, 738 mmol, 88% yield) as a yellow solid. MS (ESI): mass calcd. for C₁₁H₁₃N₃O 203.1; m/z found 204.1 [M+H]⁺. ¹H NMR (400 MHz DMSO-d₆) δ 7.10 (d, J=8.40 Hz, 2H), 7.04 (d, J=1.60 Hz, 1H), 6.85 (d, J=8.40 Hz, 2H), 5.26 (d, J=1.60 Hz, 1H), 5.20 (s, 2H), 5.10 (s, 2H), 3.71 (s, 3H).

Step D: (1E,3E)-1-Ethoxy-3-((1-(4-methoxybenzyl)-1H-pyrazol-5-yl)imino)but-1-en-1-ol. To a mixture of 1-(4-methoxybenzyl)-1H-pyrazol-5-amine (150 g, 738 mmol) and ethyl 3-oxobutanoate (135 g, 1.04 mol, 131 mL) in toluene (1.5 L) was added TsOH (2.54 g, 14.8 mmol) in one portion at 20° C. under N₂. The reaction mixture was then stirred at 70° C. for 12 hours. The reaction mixture was filtered and concentrated under reduced pressure. The resulting residue was purified by column chromatography (SiO₂, petroleum ether:ethyl acetate=10:1) to afford the title compound (82.0 g, 260 mmol, 35%) as a yellow solid. MS (ESI): mass calcd. for C₁₇H₂₁N₃O 315.2; m/z found 316.0 [M+H]⁺. ¹H NMR (400 MHz DMSO-d₆) δ 10.07 (br s, 1H), 7.45 (d, J=1.60 Hz, 1H), 7.21 (d, J=8.80 Hz, 2H), 6.83 (d, J=8.80 Hz, 2H), 5.98 (d, J=1.60 Hz, 1H), 5.16 (s, 2H), 4.77 (s, 1H), 4.16 (q, J=7.20 Hz, 2H), 3.77 (s, 3H), 1.71 (s, 3H), 1.29 (t, J=7.20 Hz, 3H).

Step E: 1-(4-Methoxybenzyl)-6-methyl-1H-pyrazolo[3,4-b]pyridin-4-ol. Dowtherm A (150 mL) was heated to 230° C., then (1E,3E)-1-ethoxy-3-((1-(4-methoxybenzyl)-1H-pyrazol-5-yl)imino)but-1-en-1-ol (40.0 g, 127 mmol, 1 eq) was added to this solution. The mixture was stirred at 240° C. for 40 min. The reaction mixture was cooled to 20° C., filtered, and concentrated under reduced pressure. The resulting residue was triturated with petroleum (200 mL) at 20° C. for 12 hours to afford the title compound (31.0 g, 115 mmol, 91%) as a yellow solid. MS (ESI): mass calcd. for C₁₅H₁₅N₃O₂ 269.1; m/z found 269.9 [M+H]⁺. ¹H NMR (400 MHz, DMSO-d₆) δ 11.36 (br s, 1H), 8.00 (s, 1H), 7.16 (d, J=8.40 Hz, 2H), 6.85 (d, J=8.00 Hz, 2H), 6.39 (s, 1H), 5.46 (s, 2H), 3.70 (s, 3H), 2.47 (s, 3H).

Step F: 4-Bromo-1-(4-methoxybenzyl)-6-methyl-1H-pyrazolo[3,4-b]pyridine. To a mixture of 1-(4-methoxybenzyl)-6-methyl-1H-pyrazolo[3,4-b]pyridin-4-ol (60 g, 223 mmol) in toluene (600 mL) and DMF (180 mL) was added POBr₃ (95.8 g, 334 mmol, 34.0 mL) in one portion at 20° C. The reaction mixture was stirred at 80° C. for 2 hours. The reaction mixture was cooled to 20° C., quenched by addition cold water (500 mL) at 20° C. and extracted with dichloromethane (500 mL*3). The combined organic layers were dried over anhydrous Na₂SO₄, filtered and concentrated under reduced pressure. The resulting residue was purified by FCC (SiO₂, petroleum ether:ethyl acetate=5:1) to afford the title compound (60.0 g, 178 mmol, 80% yield) as a light yellow solid. MS (ESI): mass calcd. for C₁₅H₁₄BrN₃O₂ 331.0; m/z found 331.8 [M+H]⁺. ¹H NMR (400 MHz, DMSO-d₆) δ 8.08 (s, 1H), 7.45 (s, 1H), 7.19 (d, J=8.80 Hz, 2H), 6.85 (d, J=8.40 Hz, 2H), 5.55 (s, 2H), 3.69 (s, 3H), 2.61 (s, 3H).

Step G. 1-(4-Methoxybenzyl)-6-methyl-4-(4,4,5,5-tetramethyl-1,3,2-dioxaborolan-2-yl)-1H-pyrazolo[3,4-b]pyridine. A solution of 4-bromo-1-(4-methoxybenzyl)-6-methyl-1H-pyrazolo[3,4-b]pyridine (600 mg, 1.81 mmol), B₂Pin₂ (688 mg, 2.71 mmol), KOAc (355 mg, 3.62 mmol) and Pd(dppf)Cl₂ (132 mg, 0.18 mmol) in DME (10 mL) was sparged with N₂ for five minutes and stirred at 90° C. for 16 hours. The reaction mixture was filtered to remove solids. The resulting filtrate was concentrated under reduced pressure. The resulting residue was purified (FCC, SiO₂, 0-17% EtOAc/petroleum ether) to afford the title compound (450 mg, 1.19 mmol, 66%). MS (ESI): mass calcd. for C₂₁H₂₆BN₃O₃ 379.2; found, 380.4 [M+H]⁺.

### Intermediate 218: 1-(4-Methoxybenzyl)-3,6-dimethyl-4-(4,4,5,5-tetramethyl-1,3,2-dioxaborolan-2-yl)-1H-pyrazolo[3,4-b]pyridine

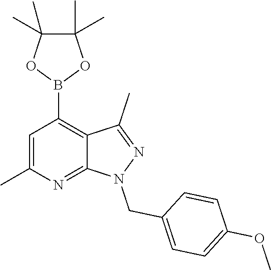

Step A. 1-(4-Methoxybenzyl)-3-methyl-1H-pyrazol-5-amine. Hydrazine hydrate (70.5 g, 1.41 mol, 68.4 mL) was slowly added (over −20 mins) to a stirred solution of (E)-but-2-enenitrile (90.0 g, 1.34 mol, 109 mL) in THF (400 mL) at 0° C. The reaction was then maintained at 25° C. for about 2 hours before 4-methoxybenzaldehyde (191 g, 1.41 mol, 171 mL) was slowly added (over −15 min). The reaction was then maintained at 25° C. for −3 hours before the THF was removed. The crude material was then diluted with n-BuOH (200 mL) and n-BuONa (128 g, 1.34 mol) in n-BuOH (200 mL) was added (over −20 mins) at 25° C. This mixture was heated to 120° C. for −3 hrs. The reaction mixture was diluted with water (500 mL) and extracted with EtOAc (3×200 mL). The organic layer was separated and treated with 1 N HCl (2×250 mL). The aqueous layer was separated and the pH was adjusted to about 14 with 50% NaOH solution. The mixture was extracted with CH₂Cl₂ (3×500 mL). The combined organic layers were washed with water (200 mL), brine (100 mL), dried over anhydrous Na₂SO₄, and concentrated under reduced pressure to yield the title compound as a light yellow oil (106 g) which was used in the next step without further purification. MS (ESI): mass calcd. for C₁₂H₁₅N₃O 217.1; m/z found 218.1 [M+H]⁺.

Step B. Ethyl (E-3-((1-(4-methoxybenzyl)-3-methyl-1H-pyrazol-5-yl)imino)butanoate.

A mixture of 1-(4-methoxybenzyl)-3-methyl-1H-pyrazol-5-amine (50.0 g, 230 mmol), TsOH (792 mg, 4.60 mmol) and ethyl 3-oxobutanoate (29.9 g, 230 mmol, 29.0 mL) in toluene (500 mL) was degassed and purged with N₂ (×3). The mixture was then stirred at 60° C. for 15 hours under a N₂ atmosphere. The reaction mixture was then cooled to 25° C. and concentrated to give a crude oil which was purified by column chromatography (SiO₂, Petroleum ether/Ethyl acetate=100/1 to 10/1) to yield the title compound as a yellow oil (26.9 g, 36%). was obtained as yellow oil. MS (ESI): mass calcd. for C₁₈H₂₃N₃O₃ 329.2; m/z found 330.3 [M+H]⁺.

Step C. 1-(4-Methoxybenzyl)-3,6-dimethyl-1H-pyrazolo[3,4-b]pyridin-4-ol. Dowtherm A™ (143 g, 442 mmol) was heated to 240° C. and then ethyl (E)-3-((1-(4-methoxybenzyl)-3-methyl-1H-pyrazol-5-yl)imino)butanoate (29.1 g, 88.5 mmol) was added. The mixture was stirred at 240° C. for 1 hour. The reaction mixture was then cooled to 25° C. and slurried with Petroleum ether (2×500 mL). The resulting precipitate was filtered and dried to yield the title compound as a white solid (23.1 g, 92% yield) which was used in the next step without further purification. MS (ESI): mass calcd. for C₁₆H₁₇N₃O₂ 283.1; m/z found 284.2 [M+H]⁺. ¹H NMR (400 MHz, CDCl₃) δ 7.16 (d, J=8.6 Hz, 2H), 6.77 (d, J=8.6 Hz, 2H), 5.79 (br s, 1H), 5.46 (s, 2H), 3.74 (s, 3H), 2.58 (s, 3H), 2.27 (s, 3H). O—H proton is not observed.

Step D. 4-Bromo-1-(4-methoxybenzyl)-3,6-dimethyl-1H-pyrazolo[3,4-b]pyridine. To a solution of 1-(4-methoxybenzyl)-3,6-dimethyl-1H-pyrazolo[3,4-b]pyridin-4-ol (30.0 g, 105 mmol) in toluene (300 mL) and DMF (90 mL) was added phosphoryl tribromide (45.5 g, 158 mmol, 16.1 mL). The mixture was stirred at 80° C. for 2 hours. The reaction mixture was then cooled to 25° C. and slowly poured into water (400 mL). The resulting reaction mixture was extracted with EtOAc (3×300 mL). The combined organics were dried, filtered, and concentrated under reduced pressure. The resulting residue was purified by FCC (SiO₂, Petroleum ether/Ethyl acetate=100/1 to 15/1) to yield the title compound as a white solid (21.5 g, 57% yield, 97% purity). MS (ESI): mass calcd. for C₁₆H₁₆BrN₃O 345.1; m/z found 346.1 [M+H]⁺. ¹H NMR (400 MHz, CDCl₃) δ 7.26-7.23 (m, 2H), 7.10 (s, 1H), 6.80 (d, J=8.6 Hz, 2H), 5.50 (s, 2H), 3.74 (s, 3H), 2.65 (s, 3H), 2.60 (s, 3H).

Step E. 1-(4-Methoxybenzyl)-3,6-dimethyl-4-(4,4,5,5-tetramethyl-1,3,2-dioxaborolan-2-yl)-1H-pyrazolo[3,4-b]pyridine. A solution of 4-bromo-1-(4-methoxybenzyl)-3,6-dimethyl-1H-pyrazolo[3,4-b]pyridine (500 mg, 1.44 mmol), 4,4,4′,4′,5,5,5′,5′-octamethyl-2,2′-bi(1,3,2-dioxaborolane (550 mg, 2.17 mmol), KOAc (285 mg, 2.90 mmol), in 1,2-dimethoxyethane (10 mL) was sparged with N₂ for 5 minutes and then treated with Pd(dtbpf)Cl₂ (95 mg, 0.15 mmol). The reaction mixture was sparged with N₂ for another 5 minutes and heated at 90° C. for 12 hours. The reaction mixture was cooled to room-temperature. The reaction mixture was filtered and the filter cake was washed with ethyl acetate (2×10 mL). The filtrate was concentrated to dryness under reduced pressure. The resulting residue was purified by FCC (eluent: petroleum ether:ethyl acetate=1:0 to 3:1) to yield the title compound as a yellow oil (350 mg, 38%). MS (ESI): mass calcd. for C₂₂H₂₈BN₃O₃ 393.2; m/z found 393.9 [M+H]⁺.

### Intermediate 219: 6-Methyl-4-(4,4,5,5-tetramethyl-1,3,2-dioxaborolan-2-yl)-1-tosyl-1H-pyrrolo[2,3-b]pyridine

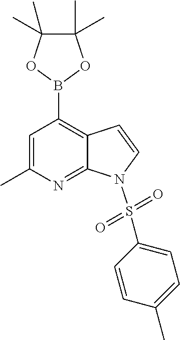 [SMILES: Cc1ccc(S(=O)(=O)n2ccc3c(B4OC(C)(C)C(C)(C)O4)cc(C)nc32)cc1]

Step A. 4-Chloro-6-methyl-1-tosyl-1H-pyrrolo[2,3-b]pyridine. TsCl (700 mg, 3.67 mmol) was added in portions to a 0° C. (ice/water) solution consisting of 4-chloro-6-methyl-1H-pyrrolo[2,3-b]pyridine (500 mg, 3.00 mmol), TEA (2.10 mL, 15.1 mmol), DMAP (91.7 mg, 0.750 mmol), and dichloromethane (10 mL). The resultant mixture was stirred for 12 hours. The reaction mixture was gradually warmed to room-temperature. The reaction mixture was poured into water (10 mL) and extracted with dichloromethane (10 mL×3). The combined organic extracts were washed with brine (10 mL), dried over anhydrous Na₂SO₄, filtered, and concentrated to dryness under reduced pressure. The resulting residue was purified by FCC (SiO₂, 0-10% EtOAc/petroleum ether) to afford the compound (290 mg, 28%) as a white solid. MS (ESI): mass calcd. for C₁₅H₁₃ClN₂O₂S 320.0 m/z; found 320.9 [M+H]⁺.

Step B. 6-Methyl-4-(4,4,5,5-tetramethyl-1,3,2-dioxaborolan-2-yl)-1-tosyl-1H-pyrrolo[2,3-b]pyridine. Pd(dppf)Cl₂ (108 mg, 0.132 mmol) was added to a mixture consisting of 4-chloro-6-methyl-1-tosyl-1H-pyrrolo[2,3-b]pyridine (290 mg, 0.904 mmol), (BPin)₂ (348 mg, 1.37 mmol), KOAc (181 mg, 1.84 mmol), and 1,2-dimethoxyethane (10 mL) under N₂. The mixture was stirred at 90° C. for 16 hours under N₂. The mixture was filtered, the filter cake was washed with ethyl acetate (30 mL×3). The filtrate was concentrated to dryness under reduced pressure and purified by FCC (SiO₂, 0-10% EtOAc/petroleum ether) to afford the product (150 mg, 40%) as a light yellow solid. MS (ESI): for C₂₁H₂₅BN₂O₄S 412.2; m/z found 413.1 [M+H]⁺. ¹H NMR (400 MHz, CDCl₃) δ 8.08 (d, J=8.5 Hz, 2H), 7.66 (d, J=4.1 Hz, 1H), 7.40 (s, 1H), 7.24 (d, J=8.3 Hz, 2H), 6.93 (d, J=3.9 Hz, 1H), 2.64 (s, 3H), 2.36 (s, 3H), 1.36 (s, 12H).

### Intermediate 220: 3-Methyl-4-(4,4,5,5-tetramethyl-1,3,2-dioxaborolan-2-yl)-1-((2-(trimethylsilyl)ethoxy)methyl)-1H-pyrazolo[3,4-b]pyridine

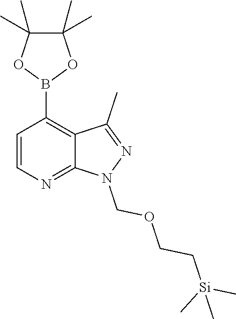

Step A. 4-Chloro-3-iodo-1H-pyrazolo[3,4-b]pyridine. NIS (9.52 g, 42.3 mmol) was added to a solution consisting of 4-chloro-1H-pyrazolo[3,4-b]pyridine (5.00 g, 32.6 mmol) and DMF (50 mL). The mixture was stirred at 80° C. for 3 h. The reaction mixture was gradually cooled to room-temperature, poured into water (300 mL). And the suspension isolated via filtration, the filter cake was washed with water (30 mL×3) and dried under reduced pressure to afford the title compound (7.0 g, 77%) as a yellow solid. MS (ESI): mass calcd. for C₆H₃ClIN₃ 278.9; m/z found 279.9 [M+H]⁺. ¹H NMR (400 MHz, DMSO-d₆) δ 14.40 (s, 1H), 8.50-8.45 (m, 1H), 7.38-7.22 (m, 1H) Step B. 4-Chloro-3-iodo-1-((2-(trimethylsilyl)ethoxy)methyl)-1H-pyrazolo[3,4-b]pyridine. Sodium hydride in mineral oil (744 mg, 60% purity, 18.6 mmol) was added in portions to a 0° C. (ice/water) solution consisting of 4-chloro-3-iodo-1H-pyrazolo[3,4-b]pyridine (4.00 g, 14.3 mmol) and THF (40 mL), The mixture was stirred at this temperature for 30 minutes. Then SEMCl (3.80 mL, 21.5 mmol) was added to the above mixture at 0° C. The resultant mixture was stirred for 12 hours. The reaction mixture was gradually warmed to room-temperature, then poured into sat·NH₄Cl (50 mL) and extracted with ethyl acetate (100 mL×3). The combined organic extracts were washed with brine (50 mL×3), dried over anhydrous Na₂SO₄, filtered, and concentrated to dryness under reduced pressure. The resulting residue was purified by FCC (SiO₂, eluent: petroleum ether:ethyl acetate=1:0 to 95:5) to afford the title compound (2 g, 34%) as a colourless oil. MS (ESI): mass calcd. for C₁₂H₁₇C₁IN₃OSi 409.0; m/z found 409.9 [M+H]⁺.

Step C. 4-Chloro-3-methyl-1-((2-(trimethylsilyl)ethoxy)methyl)-1H-pyrazolo[3,4-b]pyridine. 4-chloro-3-iodo-1-((2-(trimethylsilyl)ethoxy)methyl)-1H-pyrazolo[3,4-b]pyridine (1.50 g, 3.66 mmol), 2,4,6-trimethyl-1,3,5,2,4,6-trioxatriborinane (2.1 mL, 7.5 mmol), Cs₂CO₃ (3.57 g, 11.0 mmol) and 1,4-dioxane (12 mL), H₂O (3 mL) were added to a 40 mL reaction flask. The resultant mixture was sparged with Ar for 5 minutes and then treated with Pd(PPh₃)₄ (420 mg, 0.363 mmol). The resultant mixture was sparged with Ar for another 5 minutes and heated at 80° C. for 8 hours. The reaction mixture was cooled to room-temperature. The mixture was diluted with H₂O (20 mL) and extracted with ethyl acetate (50 mL×3). The combined organic extracts were dried over anhydrous Na₂SO₄, filtered, and concentrated to dryness under reduced pressure. The resulting residue was purified by FCC (SiO₂, eluent: petroleum ether:ethyl acetate=1:0 to 9:1) to afford the product (700 mg, 51%) as a yellow oil. MS (ESI): mass calcd. for C₁₃H₂₀ClN₃OSi 297.1; m/z found 298.1 [M+H]⁺.

Step D. 3-Methyl-4-(4,4,5,5-tetramethyl-1,3,2-dioxaborolan-2-yl)-1-((2-(trimethylsilyl)ethoxy)methyl)-1H-pyrazolo[3,4-b]pyridine. 4-Chloro-3-methyl-1-((2-(trimethylsilyl)ethoxy)methyl)-1H-pyrazolo[3,4-b]pyridine (550 mg, 1.85 mmol), 4,4,4′,4′,5,5,5′,5′-octamethyl-2,2′-bi(1,3,2-dioxaborolane) (703 mg, 2.77 mmol), potassium acetate (362 mg, 3.69 mmol), and 1,2-dimethoxyethane (10 mL) were added to a 100 mL three-necked round-bottomed flask. The resultant mixture was sparged with N₂ for 5 minutes and then treated with PdPd(dtbpf)Cl₂ (120 mg, 0.184 mmol). The resultant mixture was sparged with N₂ for another 5 minutes. The resultant mixture was heated at 90° C. for 12 hours. The reaction mixture was cooled to room-temperature. The mixture was filtered and the filter cake was washed with ethyl acetate (10 mL×3). The filtrate was concentrated to dryness under reduced pressure and purified by FCC (SiO₂, eluent: petroleum ether:ethyl acetate=1:0 to 92:8) to afford the product (560 mg) as a yellow oil. MS (ESI): mass calcd. for C₁₉H₃₂BN₃O₃Si 389.2; m/z found 390.1 [M+H]⁺.

### Intermediate 221: 6-(Difluoromethyl)-4-(4,4,5,5-tetramethyl-1,3,2-dioxaborolan-2-yl)-1-((2-(trimethylsilyl)ethoxy)methyl)-1H-pyrazolo[3,4-b]pyridine

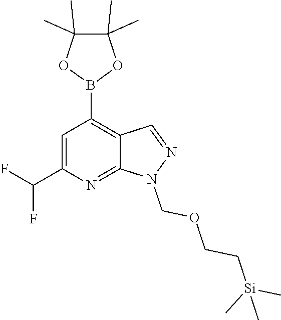 [SMILES: CC1(C)OB(c2cc(C(F)F)nc3c2cnn3COCC[Si](C)(C)C)OC1(C)C]

Step A. 4-Bromo-6-(difluoromethyl)-1H-pyrazolo[3,4-b]pyridine.

TFA (1.5 mL, 40 mmol) was added to a mixture consisting of sodium difluoromethanesulfinate (10.5 g, 76.0 mmol), 4-bromo-1H-pyrazolo[3,4-b]pyridine (5.00 g, 25.2 mmol) and H₂O (30 mL). Dichloromethane (30 mL) followed by TFA (1.5 mL, 20 mmol) was then added and stirred at room-temperature for 0.5 hour before adding t-BuOOH (21 mL, 25 mmol). The resultant mixture was stirred at room-temperature for 16 hours. The reaction mixture was poured into sat. Na₂SO₃ (150 mL) and extracted with dichloromethane (150 mL×3). The combined organic extracts were washed with brine (150 mL), dried over anhydrous Na₂SO₄, filtered, and concentrated. Purification by FCC (eluent: petroleum ether:ethyl acetate=1:0 to 5:1) yielded the title compound (3.0 g, 48%) as a yellow solid. MS (ESI): mass calcd. for C₇H₄BrF₂N₃ 248.0; m/z found 248.9 [M+H]⁺.

Step B. 4-Bromo-6-(difluoromethyl)-1-((2-(trimethylsilyl)ethoxy)methyl)-1H-pyrazolo[3,4-b]pyridine. Sodium hydride in mineral oil (505 mg, 60% purity, 12.6 mmol) was added in portions to a 0° C. (ice/water) solution consisting of 4-bromo-6-(difluoromethyl)-1H-pyrazolo[3,4-b]pyridine (2.50 g, 10.1 mmol) and THF (40 mL). The mixture was stirred at this temperature for 30 mins and then SEMCl (2.7 mL, 15 mmol) was added. The resultant mixture was stirred for 2 hours. The reaction mixture was gradually warmed to room-temperature. The mixture was poured into sat. NH₄Cl (100 mL) and extracted with ethyl acetate (100 mL×3). The combined organics were washed with brine (100 mL), dried over anhydrous Na₂SO₄, filtered, and concentrated. Purification by FCC (petroleum ether:ethyl acetate=1:0 to 10:1) yielded the title compound (2.6 g, 68%) as a yellow oil. ¹H NMR (400 MHz, CDCl₃): δ 8.15 (s, 1H), 7.71 (s, 1H), 6.87-6.51 (m, 1H), 5.87 (s, 2H), 3.71-3.64 (m, 2H), 0.97-0.92 (m, 2H), −0.02-−0.05 (m, 9H).

Step C. 6-(Difluoromethyl)-4-(4,4,5,5-tetramethyl-1,3,2-dioxaborolan-2-yl)-1-((2-(trimethylsilyl)ethoxy)methyl)-1H-pyrazolo[3,4-b]pyridine.

Pd(dppf)Cl₂ (550 mg, 0.752 mmol) was added to a mixture consisting of 4-bromo-6-(difluoromethyl)-1-((2-(trimethylsilyl)ethoxy)methyl)-1H-pyrazolo[3,4-b]pyridine (2.60 g, 6.87 mmol), (BPin)₂ (2.70 g, 10.6 mmol), KOAc (1.5 g, 15 mmol) and 1,2-dimethoxyethane (80 mL) under N₂. The mixture was stirred at 90° C. for 2.5 hours. The reaction mixture was filtered and the filter cake was washed with ethyl acetate (30 mL×3). The filtrate was concentrated to dryness under reduced pressure. The resulting residue was purified by FCC (SiO₂, petroleum ether:ethyl acetate=1:0 to 85:15) to afford the title compound (2.3 g, 65% purity) as a light yellow solid. ¹H NMR (400 MHz, CDCl₃): δ 8.42 (s, 1H), 7.90 (s, 1H), 6.85-6.54 (m, 1H), 5.89 (s, 2H), 3.68-3.59 (m, 2H), 1.42 (s, 12H), 0.96-0.88 (m, 2H), −0.03-−0.09 (m, 9H).

### Intermediate 222: 5-Fluoro-4-(4,4,5,5-tetramethyl-1,3,2-dioxaborolan-2-yl)-1-((2-(trimethylsilyl)ethoxy)methyl)-1H-pyrazolo[3,4-b]pyridine

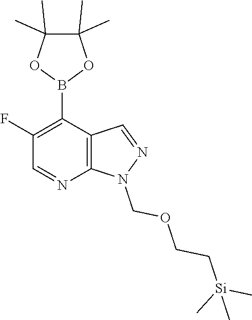 [SMILES: CC1(C)OB(c2c(F)cnc3c2cnn3COCC[Si](C)(C)C)OC1(C)C]

Pd(dppf)Cl₂·CH₂Cl₂ (0.156 g, 0.191 mmol) was added to a solution consisting of 5-fluoro-4-iodo-1-((2-(trimethylsilyl)ethoxy)methyl)-1H-pyrazolo[3,4-b]pyridine (Intermediate 204, 1.50 g, 3.81 mmol), 4,4,4′,4′,5,5,5′,5′-octamethyl (1.16 g, 4.57 mmol), KOAc (1.12 g, 11.4 mmol) and 1,4-dioxane (12 mL) under N₂ atmosphere The resultant mixture was sparged with N₂ for another 5 minutes and heated at 120° C. via microwave irradiation for 10 hours. The reaction mixture was cooled to room-temperature. The resultant mixture was concentrated to dryness under reduced pressure. The resulting residue was purified by FCC (SiO₂, 0-25% EtOAc/petroleum ether) to afford the compound (750 mg, 21%) as a white solid. MS mass calcd. for C₁₈H₂₉BFN₃O₃Si 393.2 m/z; found 311.9 [M-Pin+H]⁺. ¹H NMR (400 MHz, DMSO-d₆): δ 8.62 (d, J=1.8 Hz, 1H), 8.32-8.23 (m, 1H), 5.77 (s, 2H), 3.60-3.46 (m, 2H), 1.16 (s, 9H), 0.82-0.78 (m, 2H), −0.09 (s, 12H).

### Intermediate 223: 5-Fluoro-6-methyl-4-(4,4,5,5-tetramethyl-1,3,2-dioxaborolan-2-yl)-1-((2-(trimethylsilyl)ethoxy)methyl)-1H-pyrazolo[3,4-b]pyridine

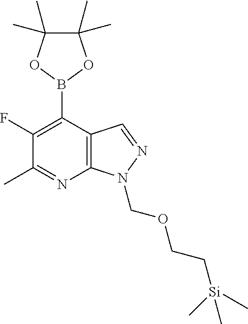 [SMILES: Cc1nc2c(cnn2COCC[Si](C)(C)C)c(B2OC(C)(C)C(C)(C)O2)c1F]

The title compound was prepared in a manner analogous to 5-fluoro-4-(4,4,5,5-tetramethyl-1,3,2-dioxaborolan-2-yl)-1-((2-(trimethylsilyl)ethoxy)methyl)-1H-pyrazolo[3,4-b]pyridine (Intermediate 222), except using 5-fluoro-4-iodo-6-methyl-1-((2-(trimethylsilyl)ethoxy)methyl)-1H-pyrazolo[3,4-b]pyridine (Intermediate 205) instead of 5-fluoro-4-iodo-1-((2-(trimethylsilyl)ethoxy)methyl)-1H-pyrazolo[3,4-b]pyridine (Intermediate 204). LCMS (ESI): mass calcd. for C₁₉H₃₁BFN₃O₃Si 407.2 m/z; found 407.9 [M+H]⁺.

### Intermediate 224: 5-(4,4,5,5-Tetramethyl-1,3,2-dioxaborolan-2-yl)pyrazolo[1,5-a]pyridine

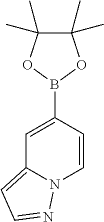 [SMILES: CC1(C)OB(c2ccn3nccc3c2)OC1(C)C]

Pd(dppf)Cl₂ (92.8 mg, 0.127 mmol) was added to a solution consisting of 5-bromopyrazolo[1,5-a]pyridine (500 mg, 2.54 mmol), 4,4,4′,4′,5,5,5′,5′-octamethyl-2,2′-bi(1,3,2-dioxaborolane) (773 mg, 3.04 mmol), KOAc (747 mg, 7.61 mmol), and 1,4-dioxane (10 mL) under N₂ atmosphere. The resultant mixture was stirred at 100° C. for 16 hours under N₂ atmosphere. The resultant mixture was concentrated to dryness under reduced pressure. The resulting residue was re-dissolved in H₂O (15 mL), and extracted with ethyl acetate (50 mL×2). The combined organic extracts were dried over anhydrous Na₂SO₄, filtered, and concentrated to dryness under reduced pressure. The resulting residue was purified by FCC (SiO₂, 10-50% EtOAc/petroleum ether) to afford the title compound (501 mg, 2.05 mmol, 81% yield) as a white solid. MS (ESI): mass calcd. for C₁₃H₁₇BN₂O₂ 244.1; m/z found 245.2 [M+H]⁺.

### Intermediate 225: 6-Methyl-5-(4,4,5,5-tetramethyl-1,3,2-dioxaborolan-2-yl)pyrazolo[1,5-a]pyridine

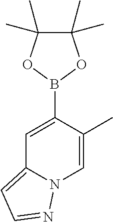 [SMILES: Cc1cn2nccc2cc1B1OC(C)(C)C(C)(C)O1]

The title compound was prepared in a manner analogous to 5-fluoro-4-(4,4,5,5-tetramethyl-1,3,2-dioxaborolan-2-yl)-1-((2-(trimethylsilyl)ethoxy)methyl)-1H-pyrazolo[3,4-b]pyridine (Intermediate 222), except using 5-bromo-6-methylpyrazolo[1,5-a]pyridine instead of 5-fluoro-4-iodo-1-((2-(trimethylsilyl)ethoxy)methyl)-1H-pyrazolo[3,4-b]pyridine (Intermediate 204). MS (ESI): mass calcd. for C₁₄H₁₉BN₂O₂ 258.2; m/z found 258.9 [M+H]⁺.

### Intermediate 226: 2-(4-Fluorophenyl)-3-(4,4,5,5-tetramethyl-1,3,2-dioxaborolan-2-yl)-6-(trifluoromethyl)-6,7-dihydro-4H-pyrazolo[5,1-c][1,4]oxazine

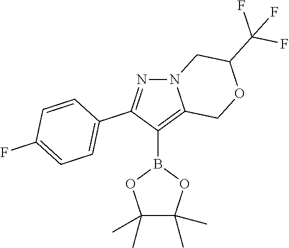 [SMILES: CC1(C)OB(c2c(-c3ccc(F)cc3)nn3c2COC(C(F)(F)F)C3)OC1(C)C]

Step A. 2-(4-Fluorophenyl)-3-iodo-6-(trifluoromethyl)-6,7-dihydro-4H-pyrazolo[5,1-c][1,4]oxazine. NIS (314 mg, 1.40 mmol) was added to a mixture consisting of 2-(4-fluorophenyl)-6-(trifluoromethyl)-6,7-dihydro-4H-pyrazolo[5,1-c][1,4]oxazine (Intermediate 79 Step C, 200 mg, 0.699 mmol) and dichloromethane (10 mL) at room-temperature. Then the reaction mixture was stirred at room-temperature for 2 hours. The reaction mixture was quenched with water (10 mL) and extracted with dichloromethane (10 mL×2). The combined organic extracts were concentrated to dryness under reduced pressure. The resulting residue was purified by FCC (SiO₂, eluent: petroleum ether:ethyl acetate=1:0 to 10:1) to afford the title compound (180 mg, 63%) as a white solid. MS (ESI): mass calcd. for C₁₃H₉F₄IN₂O 412.0; m/z found 412.9 [M+H]⁺. ¹H NMR (400 MHz, CDCl₃) δ 7.86-7.76 (m, 2H), 7.18-7.10 (m, 2H), 5.05 (d, J=15.3 Hz, 1H), 4.80 (d, J=15.4 Hz, 1H), 4.43-4.36 (m, 2H), 4.33-4.26 (m, 1H).

Step B. 2-(4-Fluorophenyl)-3-(4,4,5,5-tetramethyl-1,3,2-dioxaborolan-2-yl)-6-(trifluoromethyl)-6,7-dihydro-4H-pyrazolo[5,1-c][1,4]oxazine: Isopropylmagnesium lithium chloride (182 uL, 2 M in THF, 0.364 mmol) was dropwise to a mixture consisting of 2-(4-fluorophenyl)-3-iodo-6-(trifluoromethyl)-6,7-dihydro-4H-pyrazolo[5,1-c][1,4]oxazine (50 mg, 0.12 mmol) and THF (2 mL) at −20° C. The mixture was stirred at −20° C. for 30 mins. Then 2-isopropoxy-4,4,5,5-tetramethyl-1,3,2-dioxaborolane (74 uL, 0.36 mmol) was added to the mixture at −20° C. The resultant mixture was stirred for 16 hours. The reaction mixture was gradually warmed to room-temperature. The mixture was quenched with methanol (15 uL) and concentrated to dryness under reduced pressure. The resulting residue was purified by FCC (SiO₂, eluent: petroleum ether:ethyl acetate=1:0 to 10:1) to afford the title compound (50 mg, 92%) as a white solid. MS (ESI): mass calcd. for C₁₉H₂₁BF₄N₂O₃ 412.2; m/z found 413.0 [M+H]⁺. ¹H NMR (400 MHz, CDCl₃) δ 7.94-7.86 (m, 2H), 7.11-7.02 (m, 2H), 5.38 (d, J=16.3 Hz, 1H), 4.97 (d, J=16.1 Hz, 1H), 4.43-4.34 (m, 2H), 4.31-4.23 (m, 1H), 1.29 (s, 12H).

### Intermediate 227: 2-(5-Fluoropyridin-2-yl)-6,6-dimethyl-3-(4,4,5,5-tetramethyl-1,3,2-dioxaborolan-2-yl)-6,7-dihydro-4H-pyrazolo[5,1-c][1,4]oxazine

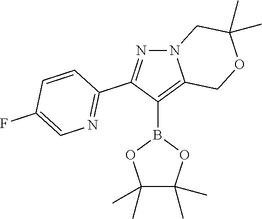 [SMILES: CC1(C)Cn2nc(-c3ccc(F)cn3)c(B3OC(C)(C)C(C)(C)O3)c2CO1]

n-BuLi (2.06 mL, 5.15 mmol, 2.5 M in hexane) was added dropwise to a −70° C. (ethanol/dry ice) solution consisting of 3-bromo-2-(5-fluoropyridin-2-yl)-6,6-dimethyl-6,7-dihydro-4H-pyrazolo[5,1-c][1,4]oxazine (Intermediate 101, 600 mg, 1.84 mmol), 2-isopropoxy-4,4,5,5-tetramethyl-1,3,2-dioxaborolane (1.13 mL, 5.54 mmol), and THF (20 mL). The reaction mixture was stirred at −78° C. for 1 hour. Then the resultant mixture was stirred for 1 hour. The reaction mixture was gradually warmed to room-temperature, then quenched with H₂O (0.33 mL). The mixture was stirred at room-temperature for 30 min, then dried over anhydrous Na₂SO₄, filtered, and concentrated to dryness under reduced pressure to afford the title product (900 mg, crude) as a white solid, which was used in the next step without further purification. MS (ESI): mass calcd. for C₁₉H₂₅BFN₃O₃ 373.2; m/z found 291.7 [M-Pin+H]⁺.

### Intermediate 228: 2-(4-Fluorophenyl)-6,6-dimethyl-3-(4,4,5,5-tetramethyl-1,3,2-dioxaborolan-2-yl)-6,7-dihydro-4H-pyrazolo[5,1-c][1,4]oxazine

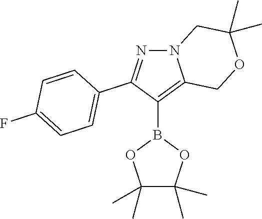 [SMILES: CC1(C)Cn2nc(-c3ccc(F)cc3)c(B3OC(C)(C)C(C)(C)O3)c2CO1]

The title compound was prepared in a manner analogous to 2-(5-fluoropyridin-2-yl)-6,6-dimethyl-3-(4,4,5,5-tetramethyl-1,3,2-dioxaborolan-2-yl)-6,7-dihydro-4H-pyrazolo[5,1-c][1,4]oxazine (Intermediate 227), except using 2-(4-fluorophenyl)-6,6-dimethyl-6,7-dihydro-4H-pyrazolo[5,1-c][1,4]oxazine (Intermediate 99) instead of 3-bromo-2-(5-fluoropyridin-2-yl)-6,6-dimethyl-6,7-dihydro-4H-pyrazolo[5,1-c][1,4]oxazine (Intermediate 101). MS (ESI): mass calcd. for C₁₄H₁₄FIN₂O 372.0; m/z found 372.9 [M+H]⁺.

### Intermediate 229: 2-(4-Fluorophenyl)-6,6-bis(methyl-d₃)-3-(4,4,5,5-tetramethyl-1,3,2-dioxaborolan-2-yl)-6,7-dihydro-4H-pyrazolo[5,1-c][1,4]oxazine

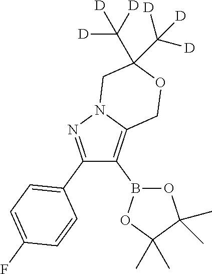 [SMILES: [2H]C([2H])([2H])C1(C([2H])([2H])[2H])Cn2nc(-c3ccc(F)cc3)c(B3OC(C)(C)C(C)(C)O3)c2CO1]

Step A: 5-(((tert-Butyldimethylsilyl)oxy)methyl)-3-(4-fluorophenyl)-1H-pyrazole. The title compound was prepared analogous to Intermediate 35 except using ethyl 3-(4-fluorophenyl)-1H-pyrazole-5-carboxylate (Intermediate 33) instead of ethyl 3-(5-fluoropyridin-2-yl)-1H-pyrazole-5-carboxylate (Intermediate 34), reaction was maintained at 0° C. instead of warming to RT. Reaction was stirred for 16 hours at room temperature instead of 30 min in Step B. MS (ESI): mass calcd. for C₁₆H₂₃FN₂OSi 306.2; m/z found 307.1 [M+H]⁺.

Step B: 2-((3-(4-Fluorophenyl)-5-(hydroxymethyl)-1H-pyrazol-1-yl)methyl)propan-1,1,1,3,3,3-d₆-2-ol. Cs₂CO₃ (4.3 g, 13 mmol) was added to a solution of 5-(((tert-butyldimethylsilyl)oxy)methyl)-3-(4-fluorophenyl)-1H-pyrazole (2.0 g, 6.5 mmol), 2-hydroxy-2-(methyl-d₃)propyl-3,3,3-d₃ 4-methylbenzenesulfonate (Intermediate 86 Step D, 2.0 g) in DMA (25 mL). The resultant mixture was heated at 135° C. for 15 hours. The reaction mixture was cooled to room-temperature. The suspension was filtered through a pad of Celite® and the pad washed with ethyl acetate (60 mL). The filtrate was concentrated. The resulting residue was purified by preparative HPLC using a Boston Uni C18, 150 mm×40 mm×5 μm column (eluent: 25% to 55% (v/v) CH₃CN and H₂O with 0.225% HCOOH) to afford pure product. The product was suspended in water (10 mL), the mixture frozen using dry ice/EtOH, and then lyophilized to dryness to afford the title compound (500 mg, 28%) as a white solid. MS (ESI): mass calcd. for C₁₄H₁₁D₆FN₂O₂ 270.2; m/z found 271.0 [M+H]⁺.

Step C: 2-(4-Fluorophenyl)-6,6-bis(methyl-d₃)-6,7-dihydro-4H-pyrazolo[5,1-c][1,4]oxazine. KOH (625 mg, 11.1 mmol) and H₂O (5 mL) were added to a solution consisting of 2-((3-(4-fluorophenyl)-5-(hydroxymethyl)-1H-pyrazol-1-yl)methyl)propan-1,1,1,3,3,3-d₆-2-ol (500 mg, 1.85 mmol) and TsCl (530 mg, 2.78 mmol) in 1,4-dioxane (10 mL).

The resultant mixture was stirred at 100° C. for 16 hours after which the resultant mixture was quenched with water (20 mL) and extracted with ethyl acetate (40 mL×3). The combined organics were dried over anhydrous Na₂SO₄, filtered, and concentrated under reduce pressure. The resulting residue was purified by FCC (SiO₂, eluent: petroleum ether:ethyl acetate=20:1 to 3:1) to afford the title compound (400 mg, 86%) as a white solid. MS (ESI): mass calcd. for C₁₄H₉D₆FN₂O 252.2; m/z found 253.0 [M+H]⁺.

Step D: 2-(4-Fluorophenyl)-3-iodo-6,6-bis(methyl-d₃)-6,7-dihydro-4H-pyrazolo[5,1-c][1,4]oxazine. To a mixture of 2-(4-fluorophenyl)-6,6-bis(methyl-d₃)-6,7-dihydro-4H-pyrazolo[5,1-c][1,4]oxazine (800 mg, 3.171 mmol) and NaHCO₃(400 mg, 4.762 mmol) in DCM (20 mL) was added N-iodosuccinimide (1.2 g, 5.3 mmol). The resulting mixture was stirred for 2 hours before being combined with an earlier batch, poured into water (20 mL) and extracted with DCM (25 mL, ×3). The combined organics were dried over anhydrous Na₂SO₄, filtered, and concentrated. The resulting residue was purified by FCC (SiO₂, eluent: petroleum ether:ethyl acetate=1:0 to 5:1) and concentrated to afford the title compound as a yellow solid (1.05 g, 84%) as a yellow solid. MS (ESI): mass calcd. for C₁₄H₈D₆FIN₂O 378.1; m/z found 379.0 [M+H]⁺.

Step E: 2-(4-Fluorophenyl)-6,6-bis(methyl-d₃)-3-(4,4,5,5-tetramethyl-1,3,2-dioxaborolan-2-yl)-6,7-dihydro-4H-pyrazolo[5,1-c][1,4]oxazine. i-PrMgCl (2.0 mL, 4.0 mmol, 2 M in THF) was added dropwise to a −5° C. (dry ice/water) solution consisting of 2-(4-fluorophenyl)-3-iodo-6,6-bis(methyl-d₃)-6,7-dihydro-4H-pyrazolo[5,1-c][1,4]oxazine (780 mg, 2.06 mmol) and dry THF (10 mL) under N₂. The reaction mixture was stirred at −5° C. (dry ice/water) for 0.5 hours, and then treated with 2-isopropoxy-4,4,5,5-tetramethyl-1,3,2-dioxaborolane (1.2 mL, 5.9 mmol). The resultant mixture was stirred for 6 hours. The reaction mixture was gradually warmed to room-temperature. The reaction mixture was quenched with sat. NH₄Cl (20 mL) and extracted with ethyl acetate (20 mL×3). The combined organics were washed with brine (50 mL), dried over anhydrous Na₂SO₄, filtered, and concentrated. The resulting residue was purified by FCC (SiO₂, eluent: petroleum ether:ethyl acetate=10:1 to 3:1) to afford the title compound (670 mg, crude) as a white solid, which was used in the next step without further purification. MS (ESI): mass calcd. for C₂₀H₂₀BD₆FN₂O₃ 378.2; m/z found 379.1 [M+H]⁺.

### Intermediate 230: Lithium 2-(2-(5-fluoropyridin-2-yl)-6,6-bis(methyl-d₃)-6,7-dihydro-4H-pyrazolo[5,1-c][1,4]oxazin-3-yl)-2-hydroxy-4,4,5,5-tetramethyl-1,3,2-dioxaborolan-2-uide

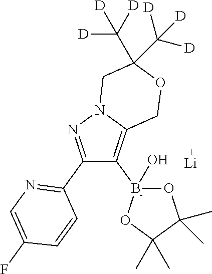

The title compound was prepared in a manner analogous to Intermediate 105, except using 3-bromo-2-(5-fluoropyridin-2-yl)-6,6-bis(methyl-d₃)-6,7-dihydro-4H-pyrazolo[5,1-c][1,4]oxazine (Intermediate 86) instead of 3-bromo-2-(5-fluoropyridin-2-yl)-6,6-dimethyl-6,7-dihydro-4H-pyrazolo[5,1-c][1,4]oxazine (Intermediate 101). MS (ESI): mass calcd. for C₁₉H₂₆BFLiN₃O₄, 403.3; m/z found 298.2 [M+H−106]⁺. ¹H NMR (400 MHz, CD₃OD) δ 8.46 (d, J=3.0 Hz, 1H), 8.34 (dd, J=9.0, 5.0 Hz, 1H), 7.63 (td, J=8.8, 3.1 Hz, 1H), 4.93 (s, 2H), 3.85 (s, 2H), 0.94-0.73 (m, 12H).

### Intermediate 231: Lithium 2-(2′-(4-fluorophenyl)-5′H,7′H-spiro[cyclopropane-1,6′-pyrazolo[5,1-b][1,3]oxazin]-3′-yl)-2-hydroxy-4,4,5,5-tetramethyl-1,3,2-dioxaborolan-2-uide

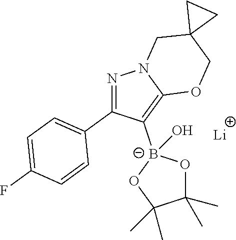 [SMILES: CC1(C)O[B-](O)(c2c(-c3ccc(F)cc3)nn3c2OCC2(CC2)C3)OC1(C)C ; [Li+]]

The title compound was prepared in a manner analogous to Intermediate 105, except using 3′-bromo-2′-(4-fluorophenyl)-5′H,7′H-spiro[cyclopropane-1,6′-pyrazolo[5,1-b][1,3]oxazine](Intermediate 195) instead of 3-bromo-2-(5-fluoropyridin-2-yl)-6,6-dimethyl-6,7-dihydro-4H-pyrazolo[5,1-c][1,4]oxazine (Intermediate 101). MS (ESI): mass calcd. for C₂₀H₂₅BFLiN₂O₄ 394.2; m/z found 371.1 [M+H−24]⁺.

### Intermediate 232: Lithium 2-(2′-(5-fluoropyridin-2-yl)-5′H,7′H-spiro[cyclopropane-1,6′-pyrazolo[5,1-b][1,3]oxazin]-3′-yl)-2-hydroxy-4,4,5,5-tetramethyl-1,3,2-dioxaborolan-2-uide

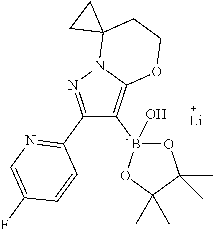

The title compound was prepared in a manner analogous to Intermediate 105, except using 3′-bromo-2′-(5-fluoropyridin-2-yl)-5′H,7′H-spiro[cyclopropane-1,6′-pyrazolo[5,1-b][1,3]oxazine](Intermediate 196) instead of 3-bromo-2-(5-fluoropyridin-2-yl)-6,6-dimethyl-6,7-dihydro-4H-pyrazolo[5,1-c][1,4]oxazine (Intermediate 101). MS (ESI): mass calcd. for C₁₉H₂₄BFLiN₃O₄ 395.2; m/z found 290.1 [M+H−106]⁺.

### Intermediate 233: 2′-(5-Fluoropyridin-2-yl)-3′-(4,4,5,5-tetramethyl-1,3,2-dioxaborolan-2-yl)-5′H,7′H-spiro[cyclopropane-1,6′-pyrazolo[5,1-b][1,3]oxazine]

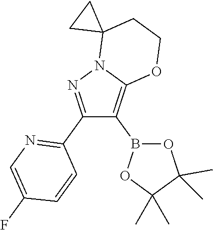 [SMILES: CC1(C)OB(c2c(-c3ccc(F)cn3)nn3c2OCCC32CC2)OC1(C)C]

The title compound was prepared in a manner analogous to Intermediate 214, except using 3′-bromo-2′-(5-fluoropyridin-2-yl)-5′H,7′H-spiro[cyclopropane-1,6′-pyrazolo[5,1-b][1,3]oxazine](Intermediate 196) instead of 3-bromo-2-(5-fluoropyridin-2-yl)-6,6-dimethyl-6,7-dihydro-4H-pyrazolo[5,1-c][1,4]oxazine (Intermediate 101). MS (ESI): mass calcd. for C₁₉H₂₃BFN₃O₃ 371.2; m/z found 290.1 [M+H−82]⁺.

### Intermediate 234: Lithium 2-(2,2-difluoro-2′-(4-fluorophenyl)-5′H,7′H-spiro[cyclopropane-1,6′-pyrazolo[5,1-b][1,3]oxazin]-3′-yl)-2-hydroxy-4,4,5,5-tetramethyl-1,3,2-dioxaborolan-2-uide

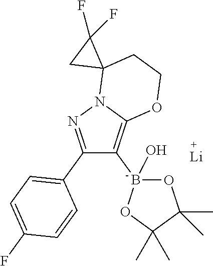

The title compound was prepared in a manner analogous to Intermediate 105, except using 3′-bromo-2,2-difluoro-2′-(4-fluorophenyl)-5′H,7′H-spiro[cyclopropane-1,6′-pyrazolo[5,1-b][1,3]oxazine] (Intermediate 197) instead of 3-bromo-2-(5-fluoropyridin-2-yl)-6,6-dimethyl-6,7-dihydro-4H-pyrazolo[5,1-c][1,4]oxazine (Intermediate 101). MS (ESI): mass calcd. for C₂₀H₂₃BF₃LiN₂O₄ 430.2; m/z found 325.1 [M+H−106]⁺.

### Intermediate 235: 5-Fluoro-4-(tributylstannyl)-1-((2-(trimethylsilyl)ethoxy)methyl)-1H-pyrazolo[3,4-b]pyridine

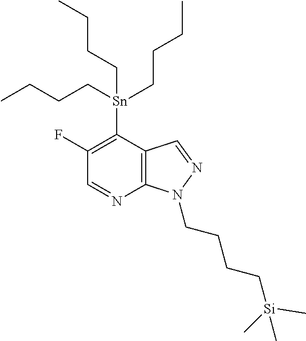 [SMILES: CCC[CH2][Sn]([CH2]CCC)([CH2]CCC)[c]1c(F)cnc2c1cnn2CCCC[Si](C)(C)C]

A solution of 5-fluoro-4-iodo-1-((2-(trimethylsilyl)ethoxy)methyl)-1H-pyrazolo[3,4-b]pyridine (Intermediate 204, 500 mg, 1.27 mmol) in dry THF (10 mL) was cooled to 0° C. iPrMgCl (2M, 700 μL, 1.40 mmol) was added dropwise and the reaction mixture was stirred for 30 minutes at 0° C. Tributyltin chloride (430 μL, 1.53 mmol) was added in one portion, and the mixture was allowed to warm to r.t. and stirred overnight. The reaction mixture was partitioned between DCM and water, the aqueous layer extracted with DCM (2×) and the combined organics were concentrated under reduced pressure. The resulting residue was purified by FCC (SiO₂, 0-40% EtOAc/hexanes) to obtain (460 mg, 0.827 mmol, 65%) of the title compound. MS (ESI): mass calcd. for C₂₄H₄₄FN₃OSiSn 557.2; found 579.7 [M+Na]⁺.

### Intermediate 236: 5-Fluoro-3,6-dimethyl-4-(tributylstannyl)-1-((2-(trimethylsilyl)ethoxy)methyl)-1H-pyrazolo[3,4-b]pyridine

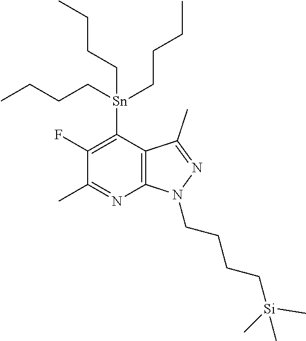 [SMILES: CCC[CH2][Sn]([CH2]CCC)([CH2]CCC)[c]1c(F)c(C)nc2c1c(C)nn2CCCC[Si](C)(C)C]

A solution of 4-bromo-5-fluoro-3,6-dimethyl-1-((2-(trimethylsilyl)ethoxy)methyl)-1H-pyrazolo[3,4-b]pyridine (Intermediate 209, 500 mg, 1.34 mmol) in dry THF (10 mL) was cooled to −78° C. nBuLi (1.6M in hexane, 920 μL, 1.40 mmol) was added dropwise and the reaction mixture was stirred for 30 minutes at −78° C. Tributyltin chloride (450 μL, 1.6 mmol) was added in one portion to the reaction mixture. The reaction mixture was allowed to warm to r.t. and stirred for 2 hours. The reaction mixture was then partitioned between DCM and water, the aqueous layer extracted 2× with DCM, and the combined organics were concentrated under reduced pressure. The resulting residue was purified by FCC (SiO₂, 0-35% EtOAc/hexanes) to obtain (562 mg, 0.962 mmol, 72%) of the title compound. MS (ESI): mass calcd. for C₂₆H₄₈FN₃OSiSn 585.3; found 608.7 [M+Na]⁺.

### Intermediate 237: 5-Fluoro-6-methyl-1-(tetrahydro-2H-pyran-2-yl)-4-(tributylstannyl)-1H-pyrazolo[3,4-b]pyridine

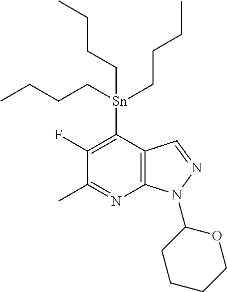 [SMILES: CCC[CH2][Sn]([CH2]CCC)([CH2]CCC)[c]1c(F)c(C)nc2c1cnn2C1CCCCO1]

The title compound was prepared in a manner analogous to 5-fluoro-4-(tributylstannyl)-1-((2-(trimethylsilyl)ethoxy)methyl)-1H-pyrazolo[3,4-b]pyridine (Intermediate 235), except using 5-fluoro-4-iodo-6-methyl-1-(tetrahydro-2H-pyran-2-yl)-1H-pyrazolo[3,4-b]pyridine (Intermediate 215) instead of 5-fluoro-4-iodo-1-((2-(trimethylsilyl)ethoxy)methyl)-1H-pyrazolo[3,4-b]pyridine (Intermediate 204), and stirred for one hour instead of overnight. MS (ESI): mass calcd. for C₂₄H₄₀FN₃OSn 525.2; found 548.2 [M+Na]⁺.

### Intermediate 238: 5-Fluoro-3-methyl-1-(tetrahydro-2H-pyran-2-yl)-4-(tributylstannyl)-1H-pyrazolo[3,4-b]pyridine

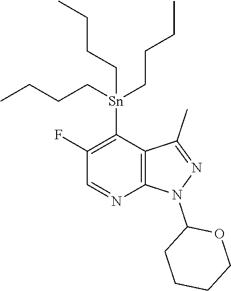 [SMILES: CCC[CH2][Sn]([CH2]CCC)([CH2]CCC)[c]1c(F)cnc2c1c(C)nn2C1CCCCO1]

The title compound was prepared in a manner analogous to 5-fluoro-3,6-dimethyl-4-(tributylstannyl)-1-((2-(trimethylsilyl)ethoxy)methyl)-1H-pyrazolo[3,4-b]pyridine (Intermediate 236), except using 5-fluoro-4-iodo-3-methyl-1-(tetrahydro-2H-pyran-2-yl)-1H-pyrazolo[3,4-b]pyridine (Intermediate 214) instead of 4-bromo-5-fluoro-3,6-dimethyl-1-((2-(trimethylsilyl)ethoxy)methyl)-1H-pyrazolo[3,4-b]pyridine (Intermediate 209). MS (ESI): mass calcd. for C₂₄H₄₀FN₃OSn 525.2; found 548.2 [M+Na]⁺.

### Intermediate 239: (S)-6-methyl-6,7-dihydro-4H-[1,2,3]oxadiazolo[4,3-c][1,4]oxazin-8-ium-3-olate

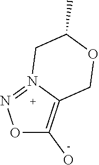

To (3S,6S)-6-methylmorpholine-3-carboxylic acid hydrochloride (500 mg, 2.75 mmol) in water (4 mL) was added HCl (37% in H₂O, 241 μL). The reaction mixture was cooled to 0° C. and sodium nitrite (209 mg, 3.03 mmol) dissolved in water (1 mL) was then added. The reaction mixture was allowed to warm to room temperature and stirred for 3 hours after which the reaction was diluted with brine. The aqueous phase was extracted 5 times with a mixture of 20% IPA in CHCl₃. The combined organic layers were dried over MgSO₄, filtered and evaporated. The residue was then taken up in diethyl ether (10 mL), cooled to 0° C., and trifluoroacetic anhydride (575 μL, 4.14 mmol) was added. The reaction mixture was allowed to warm to room temperature and stirred for 2 hours after which the reaction was quenched with potassium carbonate (600 mg, 4.34 mmol) and water (15 mL). The aqueous phase was extracted 6 times with a mixture of 20% IPA in CHCl₃. The combined organic layers were dried over MgSO₄, filtered, and evaporated to afford the title compound (256 mg, 60% yield). The material was used as is in the next step without any further purification. MS (ESI): mass calcd. for C₆H₈N₂O₃, 156.1; m/z found, 157.1 [M+H]⁺.

### Intermediate 240: (S)-3-bromo-2-(5-fluoropyridin-2-yl)-6-methyl-6,7-dihydro-4H-pyrazolo[5,1-c][1,4]oxazine

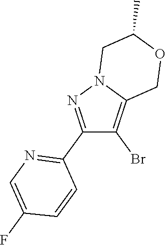 [SMILES: C[C@H]1Cn2nc(-c3ccc(F)cn3)c(Br)c2CO1]

The title compound was made in a manner analogous to Intermediate 37, Steps A-B except using (S)-6-methyl-6,7-dihydro-4H-[1,2,3]oxadiazolo[4,3-c][1,4]oxazin-8-ium-3-olate (Intermediate 239) instead of 6,7-dihydro-4H-[1,2,3]oxadiazolo[4,3-c][1,4]oxazin-8-ium-3-olate (Intermediate 2). MS (ESI): mass calcd. for C₁₂H₁₁BrFN₃O, 311.0; m/z found, 311.9 [M+H]⁺. ¹H NMR (500 MHz, DMSO-d₆) δ 8.61 (d, J=2.9 Hz, 1H), 7.89 (dd, J=8.8, 4.5 Hz, 1H), 7.78 (td, J=8.8, 3.0 Hz, 1H), 4.83 (d, J=15.2 Hz, 1H), 4.70 (d, J=15.2 Hz, 1H), 4.23 (dd, J=12.6, 3.2 Hz, 1H), 4.11-4.02 (m, 1H), 3.84-3.76 (m, 1H), 1.30 (d, J=6.2 Hz, 3H).

### Example 1: 2-(5-Fluoro-2-pyridyl)-3-(1H-pyrrolo[2,3-b]pyridin-4-yl)-6,7-dihydro-4H-pyrazolo[5,1-c][1,4]oxazine

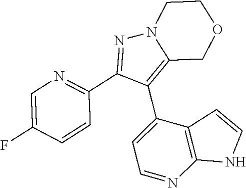 [SMILES: Fc1ccc(-c2nn3c(c2-c2ccnc4[nH]ccc24)COCC3)nc1]

Step A: 2-(5-Fluoropyridin-2-yl)-3-(1-((2-(trimethylsilyl)ethoxy)methyl)-1H-pyrrolo[2,3-b]pyridin-4-yl)-6,7-dihydro-4H-pyrazolo[5,1-c][1,4]oxazine. In a pressure vial was placed 3-bromo-2-(5-fluoropyridin-2-yl)-6,7-dihydro-4H-pyrazolo[5,1-c][1,4]oxazine (Intermediate 37, 121 mg, 0.41 mmol), 4-(4,4,5,5-tetramethyl-1,3,2-dioxaborolan-2-yl)-1-((2-(trimethylsilyl)ethoxy)methyl)-1H-pyrrolo[2,3-b]pyridine (Intermediate 21, 175 mg, 0.47 mmol), Cs₂CO₃ (397 mg, 1.2 mmol) and CataCXium® A Pd G3 (31 mg, 0.04 mmol). The tube was sealed then 2-methyl-2-butanol (3.2 mL) was added followed by water (0.8 mL). Nitrogen was bubbled through the reaction mixture for 2 minutes then heated to 90° C. for 16 hours. The reaction mixture was filtered through Celite® and the solvent was evaporated. The resulting residue was purified by FCC (SiO₂, 0-50% EtOAc in hexanes) gave the title compound (126 mg, 67%). MS (ESI): mass calcd. for C₂₄H₂₈FN₅O₂Si, 465.2; m/z found, 466.2 [M+H]⁺.

Step B: 2-(5-Fluoro-2-pyridyl)-3-(1H-pyrrolo[2,3-b]pyridin-4-yl)-6,7-dihydro-4H-pyrazolo[5,1-c][1,4]oxazine. To a solution of 2-(5-fluoropyridin-2-yl)-3-(1-((2-(trimethylsilyl)ethoxy)methyl)-1H-pyrrolo[2,3-b]pyridin-4-yl)-6,7-dihydro-4H-pyrazolo[5,1-c][1,4]oxazine (126 mg, 0.27 mmol) in dichloromethane (3.9 mL) was added trifluoroacetic acid (0.4 mL, 5.4 mmol) and the reaction mixture was stirred at room temperature for 16 hours. Solvent was evaporated and the residue was taken up in 2M NH₃ in MeOH (4 mL) and stirred at room temperature for 16 hours. Solvent was evaporated and purification by HPLC Method C; gave the title compound (58 mg, 63%). MS (ESI): mass calcd. for C₁₈H₁₄FN₅O, 335.1; m/z found, 336.1 [M+H]⁺. ¹H NMR (400 MHz, CDCl₃): δ 10.00 (s, 1H), 8.49-8.16 (m, 2H), 7.34-7.28 (m, 1H), 7.28-7.23 (m, 1H), 7.23-7.17 (m, 1H), 7.11-6.89 (m, 1H), 6.09 (s, 1H), 4.79 (s, 2H), 4.39 (t, J=5.2 Hz, 2H), 4.21 (t, J=5.2 Hz, 2H).

### Example 2: 4-[2-(5-Fluoro-2-pyridyl)-4,5,6,7-tetrahydropyrazolo[1,5-a]pyrazin-3-yl]-6-methyl-1H-pyrazolo[3,4-b]pyridine

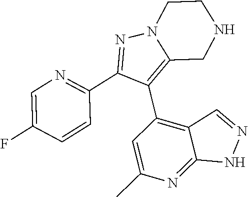 [SMILES: Cc1cc(-c2c(-c3ccc(F)cn3)nn3c2CNCC3)c2cn[nH]c2n1]

Step A: Benzyl 2-(5-fluoropyridin-2-yl)-3-(6-methyl-1H-pyrazolo[3,4-b]pyridin-4-yl)-6,7-dihydropyrazolo[1,5-a]pyrazine-5(4H)-carboxylate. The title compound was prepared in a manner analogous to Example 1, Steps A-B, except using 6-methyl-4-(4,4,5,5-tetramethyl-1,3,2-dioxaborolan-2-yl)-1-((2-(trimethylsilyl)ethoxy)methyl)-1H-pyrazolo[3,4-b]pyridine (Intermediate 26) instead of 4-(4,4,5,5-tetramethyl-1,3,2-dioxaborolan-2-yl)-1-((2-(trimethylsilyl)ethoxy)methyl)-1H-pyrrolo[2,3-b]pyridine (Intermediate 21) and benzyl 3-bromo-2-(5-fluoropyridin-2-yl)-6,7-dihydropyrazolo[1,5-a]pyrazine-5(4H)-carboxylate (Intermediate 131) instead of 3-bromo-2-(5-fluoropyridin-2-yl)-6,7-dihydro-4H-pyrazolo[5,1-c][1,4]oxazine (Intermediate 37) in Step A. MS (ESI): mass calcd. for C₂₆H₂₂FN₇O₂, 483.2; m/z found, 484.2 [M+H]⁺.

Step B: 4-[2-(5-Fluoro-2-pyridyl)-4,5,6,7-tetrahydropyrazolo[1,5-a]pyrazin-3-yl]-6-methyl-1H-pyrazolo[3,4-b]pyridine. To a solution of benzyl 2-(5-fluoropyridin-2-yl)-3-(6-methyl-1H-pyrazolo[3,4-b]pyridin-4-yl)-6,7-dihydropyrazolo[1,5-a]pyrazine-5(4H)-carboxylate (28 mg, 0.06 mmol) in methanol (2 mL) was added Pd/C (10%) Degussa type (12 mg) and the reaction mixture was stirred at room temperature under an atmosphere of H₂ for 16 hours. The palladium was filtered, and the solvent was evaporated. Purification by HPLC Method C; gave the title compound (5.7 mg, 28%). MS (ESI): mass calcd. for C₁₈H₁₆FN₇, 349.1; m/z found, 350.1 [M+H]⁺. ¹H NMR (400 MHz, CDCl₃): δ 12.21 (s, 1H), 8.29 (d, J=3.0 Hz, 1H), 7.49 (dd, J=8.8, 4.4 Hz, 1H), 7.38 (s, 1H), 7.32-7.27 (m, 1H), 6.90 (s, 1H), 4.31 (t, J=5.6 Hz, 2H), 4.09 (s, 2H), 3.42 (t, J=5.7 Hz, 2H), 2.75 (s, 3H). One of the exchangeable N—H protons is not observed.

### Example 3: 4-[2-(5-Fluoro-2-pyridyl)-4,5,6,7-tetrahydropyrazolo[1,5-a]pyridin-3-yl]-1H-pyrrolo[2,3-b]pyridine

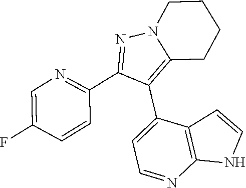 [SMILES: Fc1ccc(-c2nn3c(c2-c2ccnc4[nH]ccc24)CCCC3)nc1]

Step A: 4-(2-(5-Fluoropyridin-2-yl)-4,5,6,7-tetrahydropyrazolo[1,5-a]pyridin-3-yl)-1-(phenylsulfonyl)-1H-pyrrolo[2,3-b]pyridine. In pressure vial was placed 3-bromo-2-(5-fluoropyridin-2-yl)-4,5,6,7-tetrahydropyrazolo[1,5-a]pyridine (Intermediate 49, 101 mg, 0.34 mmol), 1-(phenylsulfonyl)-4-(4,4,5,5-tetramethyl-1,3,2-dioxaborolan-2-yl)-1H-pyrrolo[2,3-b]pyridine (197 mg, 0.51 mmol), Cs₂CO₃ (222 mg, 0.68 mmol) and XPhos Pd G3 (29 mg, 0.03 mmol). The tube was sealed then 1,4-dioxane (2.3 mL) was added followed by water (1.1 mL). Nitrogen was bubbled through the reaction mixture for 2 minutes then heated to 100° C. for 16 hours. The reaction mixture was filtered through Celite® and the solvent was evaporated. The resulting residue was purified by FCC (SiO₂, 0-100% EtOAc in hexanes) to yield the title compound (95 mg, 58%). MS (ESI): mass calcd. for C₂₅H₂₀FN₅O₂S, 473.1; m/z found, 474.1 [M+H]⁺.

Step B: 4-[2-(5-Fluoro-2-pyridyl)-4,5,6,7-tetrahydropyrazolo[1,5-a]pyridin-3-yl]-1H-pyrrolo[2,3-b]pyridine. To 4-(2-(5-fluoropyridin-2-yl)-4,5,6,7-tetrahydropyrazolo[1,5-a]pyridin-3-yl)-1-(phenylsulfonyl)-1H-pyrrolo[2,3-b]pyridine (95 mg, 0.2 mmol) was added TBAF (1M in THF, 2 mL) and the reaction was stirred at room temperature for 16 hours then heated to 45° C. for 16 hours. The solvent was evaporated under reduced pressure. The resulting residue was purified by FCC (SiO₂, 0-100% EtOAc in hexanes) to yield the title compound (41 mg, 62%). MS (ESI): mass calcd. for C₁₉H₁₆FN₅, 333.1; m/z found, 334.2 [M+H]⁺. ¹H NMR (500 MHz, CDCl₃): δ 10.61 (s, 1H), 8.41 (d, J=2.8 Hz, 1H), 8.31 (d, J=5.0 Hz, 1H), 7.23 (dd, J=3.5, 1.8 Hz, 1H), 7.19-7.15 (m, 1H), 7.12 (td, J=8.4, 2.9 Hz, 1H), 6.98 (d, J=4.9 Hz, 1H), 6.05 (dd, J=3.5, 1.4 Hz, 1H), 4.34 (t, J=6.2 Hz, 2H), 2.74 (t, J=6.3 Hz, 2H), 2.17-2.11 (m, 2H), 1.91-1.84 (m, 2H).

### Example 4: 4-(2-(4-Fluorophenyl)-5,6-dihydro-4H-pyrrolo[1,2-b]pyrazol-3-yl)-1H-pyrazolo[3,4-b]pyridine

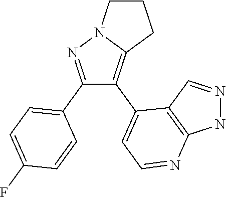 [SMILES: Fc1ccc(-c2nn3c(c2-c2ccnc4[nH]ncc24)CCC3)cc1]

The title compound was prepared in a manner analogous to Example 1, Steps A-B, except using 3-bromo-2-(4-fluorophenyl)-5,6-dihydro-4H-pyrrolo[1,2-b]pyrazole (Intermediate 38) instead of -bromo-2-(5-fluoropyridin-2-yl)-6,7-dihydro-4H-pyrazolo[5,1-c][1,4]oxazine (Intermediate 37) and 4-(4,4,5,5-tetramethyl-1,3,2-dioxaborolan-2-yl)-1-((2-(trimethylsilyl)ethoxy)methyl)-1H-pyrazolo[3,4-b]pyridine (Intermediate 23) instead of 4-(4,4,5,5-tetramethyl-1,3,2-dioxaborolan-2-yl)-1-((2-(trimethylsilyl)ethoxy)methyl)-1H-pyrrolo[2,3-b]pyridine (Intermediate 21) in Step A. MS (ESI): mass calcd. for C₁₈H₁₄FN₅, 319.1; m/z found, 320.1 [M+H]⁺. ¹H NMR (400 MHz, CD₃OD) δ 8.46 (d, J=4.8 Hz, 1H), 7.46 (s, 1H), 7.44-7.34 (m, 2H), 7.11 (d, J=4.9 Hz, 1H), 7.08-6.97 (m, 2H), 4.39-4.23 (m, 2H), 3.16-3.04 (m, 2H), 2.76 (tt, J=8.2, 6.7 Hz, 2H). N—H proton not observed

### Example 5: 4-(2-(4-Fluorophenyl)-5,6-dihydro-4H-pyrrolo[1,2-b]pyrazol-3-yl)-6-methyl-1H-pyrazolo[3,4-b]pyridine

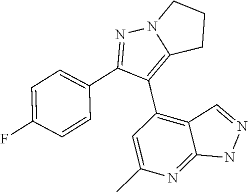 [SMILES: Cc1cc(-c2c(-c3ccc(F)cc3)nn3c2CCC3)c2cn[nH]c2n1]

The title compound was prepared in a manner analogous to Example 1, Steps A-B, except using 3-bromo-2-(4-fluorophenyl)-5,6-dihydro-4H-pyrrolo[1,2-b]pyrazole (Intermediate 38) instead of -bromo-2-(5-fluoropyridin-2-yl)-6,7-dihydro-4H-pyrazolo[5,1-c][1,4]oxazine (Intermediate 37) and 6-methyl-4-(4,4,5,5-tetramethyl-1,3,2-dioxaborolan-2-yl)-1-((2-(trimethylsilyl)ethoxy)methyl)-1H-pyrazolo[3,4-b]pyridine (Intermediate 26) instead of 4-(4,4,5,5-tetramethyl-1,3,2-dioxaborolan-2-yl)-1-((2-(trimethylsilyl)ethoxy)methyl)-1H-pyrrolo[2,3-b]pyridine (Intermediate 21) in Step A. MS (ESI): mass calcd. for C₁₉H₁₆FN₅, 333.1; m/z found, 334.1 [M+H]⁺. ¹H NMR (400 MHz, CD₃OD) δ 7.44-7.35 (m, 2H), 7.34 (s, 1H), 7.09-6.98 (m, 3H), 4.30 (t, J=7.3 Hz, 2H), 3.15-3.04 (m, 2H), 2.86-2.66 (m, 2H), 2.63 (s, 3H). N—H proton not observed.

### Example 6: 4-(2-(5-Fluoropyridin-2-yl)-5,6-dihydro-4H-pyrrolo[1,2-b]pyrazol-3-yl)-1H-pyrrolo[2,3-b]pyridine

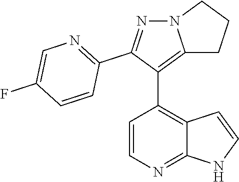 [SMILES: Fc1ccc(-c2nn3c(c2-c2ccnc4[nH]ccc24)CCC3)nc1]

The title compound was prepared in a manner analogous to Example 1, Step A, except using 3-bromo-2-(5-fluoropyridin-2-yl)-5,6-dihydro-4H-pyrrolo[1,2-b]pyrazole (Intermediate 39) instead of 3-bromo-2-(5-fluoropyridin-2-yl)-6,7-dihydro-4H-pyrazolo[5,1-c][1,4]oxazine (Intermediate 37) and using 4-(4,4,5,5-tetramethyl-1,3,2-dioxaborolan-2-yl)-1H-pyrrolo[2,3-b]pyridine instead of 4-(4,4,5,5-tetramethyl-1,3,2-dioxaborolan-2-yl)-1-((2-(trimethylsilyl)ethoxy)methyl)-1H-pyrrolo[2,3-b]pyridine (Intermediate 21). MS (ESI): mass calcd. for C₁₈H₁₄FN₅, 319.1; m/z found, 320.1 [M+H]⁺. ¹H NMR (500 MHz, DMSO-d₆) δ 11.49 (br s, 1H), 8.28 (d, J=2.25 Hz, 1H), 8.10 (d, J=4.88 Hz, 1H), 7.77-7.65 (m, 2H), 7.28 (dd, J=3.38, 2.50 Hz, 1H), 6.86 (d, J=4.88 Hz, 1H), 5.86 (dd, J=3.50, 1.88 Hz, 1H), 4.24 (t, J=7.19 Hz, 2H), 2.92 (t, J=7.32 Hz, 2H), 2.66-2.56 (m, 2H).

### Example 7: 4-[2-(5-Fluoro-2-pyridyl)-5,6-dihydro-4H-pyrrolo[1,2-b]pyrazol-3-yl]-1H-pyrazolo[3,4-b]pyridine

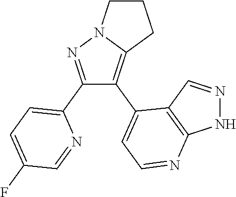 [SMILES: Fc1ccc(-c2nn3c(c2-c2ccnc4[nH]ncc24)CCC3)nc1]

The title compound was prepared in a manner analogous to Example 1, Steps A-B, except using 3-bromo-2-(5-fluoropyridin-2-yl)-5,6-dihydro-4H-pyrrolo[1,2-b]pyrazole (Intermediate 39) instead of 3-bromo-2-(5-fluoropyridin-2-yl)-6,7-dihydro-4H-pyrazolo[5,1-c][1,4]oxazine (Intermediate 37) and 4-(4,4,5,5-tetramethyl-1,3,2-dioxaborolan-2-yl)-1-((2-(trimethylsilyl)ethoxy)methyl)-1H-pyrazolo[3,4-b]pyridine (Intermediate 23) instead of 4-(4,4,5,5-tetramethyl-1,3,2-dioxaborolan-2-yl)-1-((2-(trimethylsilyl)ethoxy)methyl)-1H-pyrrolo[2,3-b]pyridine (Intermediate 21). MS (ESI): mass calcd. for C₁₇H₁₃FN₆, 320.1; m/z found, 321.0 [M+H]⁺. ¹H NMR (400 MHz, DMSO-d₆) δ 13.50 (br s, 1H), 8.41 (d, J=4.9 Hz, 1H), 8.29-8.24 (m, 1H), 7.88-7.73 (m, 2H), 7.38 (s, 1H), 7.01 (d, J=4.9 Hz, 1H), 4.25 (t, J=7.2 Hz, 2H), 3.00 (t, J=7.2 Hz, 2H), 2.68-2.57 (m, 2H).

### Example 8: 4-[2-(5-Fluoro-2-pyridyl)-5,6-dihydro-4H-pyrrolo[1,2-b]pyrazol-3-yl]-6-methyl-1H-pyrazolo[3,4-b]pyridine

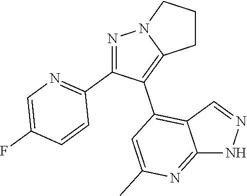 [SMILES: Cc1cc(-c2c(-c3ccc(F)cn3)nn3c2CCC3)c2cn[nH]c2n1]

The title compound was prepared in a manner analogous to Example 1, Steps A-B, except using 6-methyl-4-(4,4,5,5-tetramethyl-1,3,2-dioxaborolan-2-yl)-1-((2-(trimethylsilyl)ethoxy)methyl)-1H-pyrazolo[3,4-b]pyridine (Intermediate 26) instead of 4-(4,4,5,5-tetramethyl-1,3,2-dioxaborolan-2-yl)-1-((2-(trimethylsilyl)ethoxy)methyl)-1H-pyrrolo[2,3-b]pyridine (Intermediate 21) and 3-bromo-2-(5-fluoropyridin-2-yl)-5,6-dihydro-4H-pyrrolo[1,2-b]pyrazole (Intermediate 39) instead of 3-bromo-2-(5-fluoropyridin-2-yl)-6,7-dihydro-4H-pyrazolo[5,1-c][1,4]oxazine (Intermediate 37) in Step A. MS (ESI): mass calcd. for C₁₈H₁₅FN₆, 334.1; m/z found, 335.1 [M+H]⁺. ¹H NMR (500 MHz, CDCl₃): δ 12.34 (s, 1H), 8.30 (d, J=2.9 Hz, 1H), 7.58 (dd, J=8.7, 4.4 Hz, 1H), 7.38 (s, 1H), 7.33 (td, J=8.4, 2.9 Hz, 1H), 6.95 (s, 1H), 4.33 (t, J=7.3 Hz, 2H), 3.05 (t, J=7.3 Hz, 2H), 2.78-2.68 (m, 5H).

### Example 9: 5-Fluoro-4-(2-(5-fluoropyridin-2-yl)-5,6-dihydro-4H-pyrrolo[1,2-b]pyrazol-3-yl)-6-methyl-1H-pyrazolo[3,4-b]pyridine

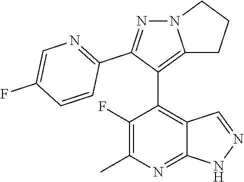 [SMILES: Cc1nc2[nH]ncc2c(-c2c(-c3ccc(F)cn3)nn3c2CCC3)c1F]

The title compound was prepared in a manner analogous to Example 157, Steps A-B, except using 3-bromo-2-(5-fluoropyridin-2-yl)-5,6-dihydro-4H-pyrrolo[1,2-b]pyrazole (Intermediate 39) instead of 3-bromo-2-(5-fluoropyridin-2-yl)-6,6-dimethyl-6,7-dihydro-4H-pyrazolo[5,1-c][1,4]oxazine (Intermediate 101) in Step A. LCMS (ESI): mass calcd. for C₁₈H₁₄F₂N₆ 352.1 m/z, found 353.0 [M+H]⁺. ¹H NMR (400 MHz, DMSO-d₆) δ 13.47 (s, 1H), 8.22 (d, J=2.8 Hz, 1H), 7.94-7.87 (m, 1H), 7.81-7.71 (m, 1H), 7.56 (s, 1H), 4.27 (t, J=7.3 Hz, 2H), 2.95-2.84 (m, 2H), 2.66-2.57 (m, 2H), 2.56-2.52 (m, 3H).

### Example 10: (S)-4-(5-Fluoro-2-(4-fluorophenyl)-5,6-dihydro-4H-pyrrolo[1,2-b]pyrazol-3-yl)pyridin-2-amine

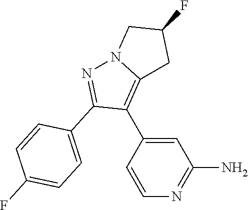 [SMILES: Nc1cc(-c2c(-c3ccc(F)cc3)nn3c2C[C@H](F)C3)ccn1]

(S)-3-Bromo-5-fluoro-2-(4-fluorophenyl)-5,6-dihydro-4H-pyrrolo[1,2-b]pyrazole (Intermediate 41, 35 mg, 0.117 mmol), 4-(4,4,5,5-tetramethyl-1,3,2-dioxaborolan-2-yl)pyridin-2-amine (103 mg, 0.468 mmol), CataXcium Pd G4 (4.3 mg, 0.0059 mmol), and Cs₂CO₃ (116 mg, 0.351 mmol) were dissolved in a mixture of 2-methyl-2-butanol (2 mL) and water (1 mL). The biphasic mixture was stirred at 90° C. for 2 hours, then stirred overnight at 110° C. The reaction mixture was allowed to cool to room temperature, partitioned between DCM and water, and the aqueous layer was extracted 2× with DCM. The combined organics were concentrated and the residue was purified HPLC Method D: to obtain 19.7 mg (0.631 mmol, 54% yield) of the title compound. MS (ESI): mass calcd. for C₁₇H₁₄F₂N₄, 312.1; m/z found, 313.1 [M+H]⁺. ¹H NMR (400 MHz, CDCl₃): δ 7.91 (d, J=5.4, 0.7 Hz, 1H), 7.45-7.34 (m, 2H), 7.00-6.91 (m, 2H), 6.41 (dd, J=5.3, 1.5 Hz, 1H), 6.27-6.19 (m, 1H), 5.87-5.59 (m, 1H), 4.43 (d, J=3.0 Hz, 1H), 4.38-4.34 (m, 1H), 4.26 (s, 2H), 3.43-3.14 (m, 2H).

### Example 11: (S)-4-(5-Fluoro-2-(4-fluorophenyl)-5,6-dihydro-4H-pyrrolo[1,2-b]pyrazol-3-yl)-1H-pyrazolo[3,4-b]pyridine

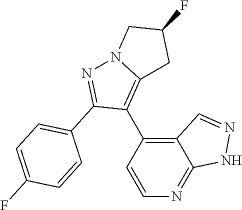 [SMILES: Fc1ccc(-c2nn3c(c2-c2ccnc4[nH]ncc24)C[C@H](F)C3)cc1]

The title compound was prepared in a manner analogous to Example 27, Steps A-B, except using (S)-3-bromo-5-fluoro-2-(4-fluorophenyl)-5,6-dihydro-4H-pyrrolo[1,2-b]pyrazole (Intermediate 41) instead of (4aR,5aR)-3-Bromo-2-(5-fluoropyridin-2-yl)-4,4a,5,5a-tetrahydrocyclopropa[4,5]pyrrolo[1,2-b]pyrazole (Intermediate 47) and 4-(4,4,5,5-tetramethyl-1,3,2-dioxaborolan-2-yl)-1-((2-(trimethylsilyl)ethoxy)methyl)-1H-pyrazolo[3,4-b]pyridine (Intermediate 23) instead of 6-methyl-4-(4,4,5,5-tetramethyl-1,3,2-dioxaborolan-2-yl)-1-((2-(trimethylsilyl)ethoxy)methyl)-1H-pyrazolo[3,4-b]pyridine (Intermediate 26). MS (ESI): mass calcd. for C₁₈H₁₃F₂N₅, 337.1; m/z found, 338.1 [M+H]⁺. ¹H NMR (400 MHz, CDCl₃): δ 10.82 (s, 1H), 8.43 (d, J=4.8 Hz, 1H), 7.47 (s, 1H), 7.35-7.28 (m, 2H), 6.94-6.81 (m, 3H), 5.92-5.66 (m, 1H), 4.52 (d, J=3.0 Hz, 1H), 4.45 (dd, J=3.0, 1.6 Hz, 1H), 3.43-3.15 (m, 2H).

### Example 12: (S)-4-(5-Fluoro-2-(4-fluorophenyl)-5,6-dihydro-4H-pyrrolo[1,2-b]pyrazol-3-yl)-6-methyl-1H-pyrazolo[3,4-b]pyridine

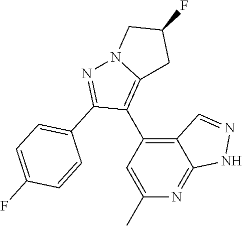 [SMILES: Cc1cc(-c2c(-c3ccc(F)cc3)nn3c2C[C@H](F)C3)c2cn[nH]c2n1]

The title compound was prepared in a manner analogous to (4aR,5aR)-2-(5-fluoropyridin-2-yl)-3-(6-methyl-1H-pyrazolo[3,4-b]pyridin-4-yl)-4,4a,5,5a-tetrahydrocyclopropa[4,5]pyrrolo[1,2-b]pyrazole (Example 27), Steps A-B, except using (S)-3-Bromo-5-fluoro-2-(4-fluorophenyl)-5,6-dihydro-4H-pyrrolo[1,2-b]pyrazole (Intermediate 41) instead of (4aR,5aR)-3-Bromo-2-(5-fluoropyridin-2-yl)-4,4a,5,5a-tetrahydrocyclopropa[4,5]pyrrolo[1,2-b]pyrazole (Intermediate 47). MS (ESI): mass calcd. for C₁₉H₁₅F₂N₅, 351.1; m/z found, 352.2 [M+H]⁺. ¹H NMR (400 MHz, CDCl₃): δ 11.16 (s, 1H), 7.37 (s, 1H), 7.36-7.28 (m, 2H), 6.87 (t, J=8.7 Hz, 2H), 6.80 (s, 1H), 5.89-5.61 (m, 1H), 4.57-4.31 (m, 2H), 3.43-3.15 (m, 2H), 2.60 (s, 3H).

### Example 13: 4-(5-Fluoro-2-(5-fluoropyridin-2-yl)-5,6-dihydro-4H-pyrrolo[1,2-b]pyrazol-3-yl)-6-methyl-1H-pyrazolo[3,4-b]pyridine

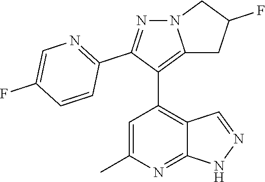 [SMILES: Cc1cc(-c2c(-c3ccc(F)cn3)nn3c2CC(F)C3)c2cn[nH]c2n1]

The title compound was prepared in a manner analogous to Example 1, Steps A-B, except using 3-bromo-5-fluoro-2-(5-fluoropyridin-2-yl)-5,6-dihydro-4H-pyrrolo[1,2-b]pyrazole (Intermediate 40) instead of 3-bromo-2-(5-fluoropyridin-2-yl)-6,7-dihydro-4H-pyrazolo[5,1-c][1,4]oxazine (Intermediate 37) and 6-methyl-4-(4,4,5,5-tetramethyl-1,3,2-dioxaborolan-2-yl)-1-((2-(trimethylsilyl)ethoxy)methyl)-1H-pyrazolo[3,4-b]pyridine (Intermediate 26) instead of 4-(4,4,5,5-tetramethyl-1,3,2-dioxaborolan-2-yl)-1-((2-(trimethylsilyl)ethoxy)methyl)-1H-pyrazolo[3,4-b]pyridine (Intermediate 23) in Step A. MS (ESI): mass calcd. for C₁₈H₁₄F₂N₆, 352.1; m/z found, 353.2 [M+H]⁺. ¹H NMR (400 MHz, DMSO-d₆) δ 13.27 (s, 1H), 8.28 (d, J=2.88 Hz, 1H), 7.88-7.82 (m, 1H), 7.81-7.74 (m, 1H), 7.17 (s, 1H), 6.98 (s, 1H), 6.02-5.85 (m, 1H), 4.70-4.55 (m, 1H), 4.54-4.42 (m, 1H), 3.64-3.47 (m, 1H), 3.26-3.13 (m, 1H), 2.56 (s, 3H).

### Example 14: 4-(4,4-Difluoro-2-(5-fluoropyridin-2-yl)-5,6-dihydro-4H-pyrrolo[1,2-b]pyrazol-3-yl)-1H-pyrazolo[3,4-b]pyridine

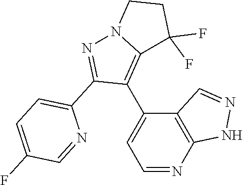 [SMILES: Fc1ccc(-c2nn3c(c2-c2ccnc4[nH]ncc24)C(F)(F)CC3)nc1]

The title compound was prepared in a manner analogous to Example 1, Steps A-B, except using 3-bromo-4,4-difluoro-2-(5-fluoropyridin-2-yl)-5,6-dihydro-4H-pyrrolo[1,2-b]pyrazole (Intermediate 142) instead of 3-bromo-2-(5-fluoropyridin-2-yl)-6,7-dihydro-4H-pyrazolo[5,1-c][1,4]oxazine (Intermediate 37) and 4-(4,4,5,5-tetramethyl-1,3,2-dioxaborolan-2-yl)-1-((2-(trimethylsilyl)ethoxy)methyl)-1H-pyrazolo[3,4-b]pyridine (Intermediate 23) instead of 4-(4,4,5,5-tetramethyl-1,3,2-dioxaborolan-2-yl)-1-((2-(trimethylsilyl)ethoxy)methyl)-1H-pyrrolo[2,3-b]pyridine (Intermediate 21) in Step A. MS (ESI): mass calcd. for C₁₇H₁₁F₃N₆ 356.1; m/z found 357.0 [M+H]⁺. ¹H NMR (400 MHz, DMSO-d₆) δ 13.62 (br s, 1H), 8.53 (d, J=4.5 Hz, 1H), 8.34-8.19 (m, 1H), 7.98-7.87 (m, 1H), 7.86-7.74 (m, 1H), 7.35-7.19 (m, 1H), 7.12 (d, J=4.5 Hz, 1H), 4.67-4.53 (m, 2H), 3.32-3.29 (m, 2H).

### Example 15: 4-[(6S)-2-(4-Fluorophenyl)-6-methyl-5,6-dihydro-4H-pyrrolo[1,2-b]pyrazol-3-yl]-1H-pyrazolo[3,4-b]pyridine

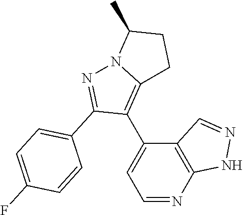 [SMILES: C[C@H]1CCc2c(-c3ccnc4[nH]ncc34)c(-c3ccc(F)cc3)nn21]

The title compound was made in a manner analogous to Example 1, Steps A-B except using (S)-3-bromo-2-(4-fluorophenyl)-6-methyl-5,6-dihydro-4H-pyrrolo[1,2-b]pyrazole (Intermediate 42) instead of 3-bromo-2-(5-fluoropyridin-2-yl)-6,7-dihydro-4H-pyrazolo[5,1-c][1,4]oxazine (Intermediate 37) and 4-(4,4,5,5-tetramethyl-1,3,2-dioxaborolan-2-yl)-1-((2-(trimethylsilyl)ethoxy)methyl)-1H-pyrazolo[3,4-b]pyridine (Intermediate 23) instead of 4-(4,4,5,5-tetramethyl-1,3,2-dioxaborolan-2-yl)-1-((2-(trimethylsilyl)ethoxy)methyl)-1H-pyrrolo[2,3-b]pyridine (Intermediate 21) and also microwave irradiation at 80° C. for 1 hr rather than conventional heating in Step A. MS (ESI): mass calcd. for C₁₉H₁₆FN₅ 333.1 m/z found 334.0 [M+H]⁺. ¹H NMR (400 MHz, DMSO-d₆) δ 13.57 (br s, 1H), 8.43 (d, J=4.8 Hz, 1H), 7.48 (s, 1H), 7.39-7.22 (m, 2H), 7.16-7.04 (m, 2H), 6.99 (d, J=4.8 Hz, 1H), 4.61-4.45 (m, 1H), 3.08-2.87 (m, 2H), 2.86-2.72 (m, 1H), 2.29-2.13 (m, 1H), 1.52 (d, J=6.3 Hz, 3H).

### Example 16: 4-[(6R)-2-(4-Fluorophenyl)-6-methyl-5,6-dihydro-4H-pyrrolo[1,2-b]pyrazol-3-yl]-1H-pyrazolo[3,4-b]pyridine

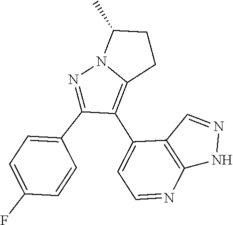 [SMILES: C[C@@H]1CCc2c(-c3ccnc4[nH]ncc34)c(-c3ccc(F)cc3)nn21]

The title compound was prepared in a manner analogous to Example 1, Steps A-B except using (R)-3-bromo-2-(4-fluorophenyl)-6-methyl-5,6-dihydro-4H-pyrrolo[1,2-b]pyrazole (Intermediate 43) instead of 3-bromo-2-(5-fluoropyridin-2-yl)-6,7-dihydro-4H-pyrazolo[5,1-c][1,4]oxazine (Intermediate 37) and 4-(4,4,5,5-tetramethyl-1,3,2-dioxaborolan-2-yl)-1-((2-(trimethylsilyl)ethoxy)methyl)-1H-pyrazolo[3,4-b]pyridine (Intermediate 23) instead of 4-(4,4,5,5-tetramethyl-1,3,2-dioxaborolan-2-yl)-1-((2-(trimethylsilyl)ethoxy)methyl)-1H-pyrrolo[2,3-b]pyridine (Intermediate 21) and also microwave irradiation at 100° C. for 1 h rather than conventional heating in Step A. MS (ESI): mass calcd. for C₁₉H₁₆FN₅ 333.1 m/z found 334.0 [M+H]⁺. ¹H NMR (400 MHz, DMSO-d₆): δ 13.57 (s, 1H), 8.42 (d, J=4.8 Hz, 1H), 7.48 (d, J=1.2 Hz, 1H), 7.38-7.29 (m, 2H), 7.15-7.06 (m, 2H), 6.98 (d, J=4.8 Hz, 1H), 4.52 (sxt, J=6.6 Hz, 1H), 3.08-2.98 (m, 1H), 2.98-2.88 (m, 1H), 2.79 (dtd, J=4.3, 8.2, 12.1 Hz, 1H), 2.20 (tdd, J=6.6, 8.9, 12.8 Hz, 1H), 1.52 (d, J=6.3 Hz, 3H).

### Example 17: 4-[(6S)-2-(4-Fluorophenyl)-6-methyl-5,6-dihydro-4H-pyrrolo[1,2-b]pyrazol-3-yl]-6-methyl-1H-pyrazolo[3,4-b]pyridine

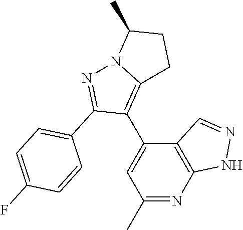 [SMILES: Cc1cc(-c2c(-c3ccc(F)cc3)nn3c2CC[C@@H]3C)c2cn[nH]c2n1]

The title compound was prepared in a manner analogous to Example 1, Steps A-B, except using (S)-3-bromo-2-(4-fluorophenyl)-6-methyl-5,6-dihydro-4H-pyrrolo[1,2-b]pyrazole (Intermediate 42) instead of 3-bromo-2-(5-fluoropyridin-2-yl)-6,7-dihydro-4H-pyrazolo[5,1-c][1,4]oxazine (Intermediate 37) and 6-methyl-4-(4,4,5,5-tetramethyl-1,3,2-dioxaborolan-2-yl)-1-((2-(trimethylsilyl)ethoxy)methyl)-1H-pyrazolo[3,4-b]pyridine (Intermediate 26) instead of 4-(4,4,5,5-tetramethyl-1,3,2-dioxaborolan-2-yl)-1-((2-(trimethylsilyl)ethoxy)methyl)-1H-pyrrolo[2,3-b]pyridine (Intermediate 21) and also microwave irradiation at 80° C. for 1 hr rather than conventional heating in Step A. MS (ESI): mass calcd. for C₂₀H₁₈FN₅ 347.2 m/z found 348.0 [M+H]⁺. ¹H NMR (400 MHz, DMSO-d₆) δ 13.33 (s, 1H), 7.37-7.31 (m, 2H), 7.29 (d, J=1.0 Hz, 1H), 7.17-7.05 (m, 2H), 6.92 (s, 1H), 4.65-4.27 (m, 1H), 3.09-2.98 (m, 1H), 2.98-2.87 (m, 1H), 2.85-2.74 (m, 1H), 2.52 (s, 3H), 2.27-2.12 (m, 1H), 1.52 (d, J=6.3 Hz, 3H).

### Example 18: 4-[(6R)-2-(4-Fluorophenyl)-6-methyl-5,6-dihydro-4H-pyrrolo[1,2-b]pyrazol-3-yl]-6-methyl-1H-pyrazolo[3,4-b]pyridine

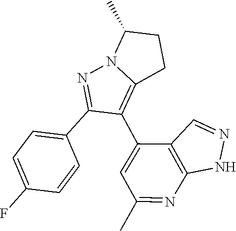 [SMILES: Cc1cc(-c2c(-c3ccc(F)cc3)nn3c2CC[C@H]3C)c2cn[nH]c2n1]

The title compound was prepared in a manner analogous to Example 1, Steps A-B, except using (R)-3-bromo-2-(4-fluorophenyl)-6-methyl-5,6-dihydro-4H-pyrrolo[1,2-b]pyrazole (Intermediate 43) instead of 3-bromo-2-(5-fluoropyridin-2-yl)-6,7-dihydro-4H-pyrazolo[5,1-c][1,4]oxazine (Intermediate 37) and 6-methyl-4-(4,4,5,5-tetramethyl-1,3,2-dioxaborolan-2-yl)-1-((2-(trimethylsilyl)ethoxy)methyl)-1H-pyrazolo[3,4-b]pyridine (Intermediate 26) instead of 4-(4,4,5,5-tetramethyl-1,3,2-dioxaborolan-2-yl)-1-((2-(trimethylsilyl)ethoxy)methyl)-1H-pyrrolo[2,3-b]pyridine (Intermediate 21) and also microwave irradiation at 100° C. for 1 h rather than conventional heating in Step A. MS (ESI): mass calcd. for C₂₀H₁₈FN₅ 347.2 m/z found 348.0 [M+H]⁺. ¹H NMR (400 MHz, DMSO-d₆) δ 13.32 (s, 1H), 7.37-7.31 (m, 2H), 7.30 (d, J=1.3 Hz, 1H), 7.15-7.07 (m, 2H), 6.92 (s, 1H), 4.52 (sxt, J=6.6 Hz, 1H), 3.09-2.98 (m, 1H), 2.97-2.87 (m, 1H), 2.80 (dtd, J=4.2, 8.2, 12.1 Hz, 1H), 2.53 (s, 3H), 2.20 (tdd, J=6.6, 8.9, 12.8 Hz, 1H), 1.52 (d, J=6.4 Hz, 3H).

### Example 19: 4-[(6S)-2-(5-Fluoro-2-pyridyl)-6-methyl-5,6-dihydro-4H-pyrrolo[1,2-b]pyrazol-3-yl]-1H-pyrazolo[3,4-b]pyridine

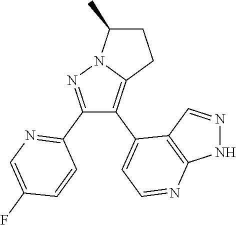 [SMILES: C[C@H]1CCc2c(-c3ccnc4[nH]ncc34)c(-c3ccc(F)cn3)nn21]

The title compound was prepared in a manner analogous to Example 1, Steps A-B, except using (6S)-3-bromo-2-(5-fluoro-2-pyridyl)-6-methyl-5, 6-dihydro-4H-pyrrolo[1,2-b]pyrazole (Intermediate 44) instead of 3-bromo-2-(5-fluoropyridin-2-yl)-6,7-dihydro-4H-pyrazolo[5,1-c][1,4]oxazine (Intermediate 37) and 4-(4,4,5,5-tetramethyl-1,3,2-dioxaborolan-2-yl)-1-((2-(trimethylsilyl)ethoxy)methyl)-1H-pyrazolo[3,4-b]pyridine (Intermediate 23) instead of 4-(4,4,5,5-tetramethyl-1,3,2-dioxaborolan-2-yl)-1-((2-(trimethylsilyl)ethoxy)methyl)-1H-pyrrolo[2,3-b]pyridine (Intermediate 21) in Step A. MS (ESI): mass calcd. for C₁₈H₁₅FN₆, 334.1; m/z found, 335.1 [M+H]⁺. ¹H NMR (500 MHz, CD₃OD) δ 8.43 (d, J=4.8 Hz, 1H), 8.23 (d, J=2.9 Hz, 1H), 7.72 (dd, J=8.7, 4.5 Hz, 1H), 7.60 (td, J=8.6, 2.9 Hz, 1H), 7.40 (s, 1H), 7.09 (dd, J=4.8, 2.2 Hz, 1H), 4.59 (q, J=6.6 Hz, 1H), 3.15-2.97 (m, 2H), 2.92 (dddd, J=12.1, 8.6, 7.4, 4.6 Hz, 1H), 2.32 (ddt, J=13.0, 9.0, 6.6 Hz, 1H), 1.63 (dd, J=6.4, 1.6 Hz, 3H). N—H proton is not observed.

### Example 20: 4-[(6R)-2-(5-Fluoro-2-pyridyl)-6-methyl-5,6-dihydro-4H-pyrrolo[1,2-b]pyrazol-3-yl]-1H-pyrazolo[3,4-b]pyridine

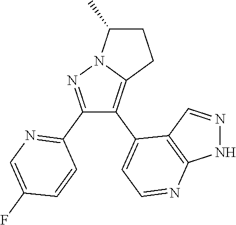 [SMILES: C[C@@H]1CCc2c(-c3ccnc4[nH]ncc34)c(-c3ccc(F)cn3)nn21]

The title compound was prepared in a manner analogous to Example 1, Steps A-B, except using (R)-3-bromo-2-(5-fluoropyridin-2-yl)-6-methyl-5,6-dihydro-4H-pyrrolo[1,2-b]pyrazole (Intermediate 45) instead of 3-bromo-2-(5-fluoropyridin-2-yl)-6,7-dihydro-4H-pyrazolo[5,1-c][1,4]oxazine (Intermediate 37) and 4-(4,4,5,5-tetramethyl-1,3,2-dioxaborolan-2-yl)-1-((2-(trimethylsilyl)ethoxy)methyl)-1H-pyrazolo[3,4-b]pyridine (Intermediate 23) instead of 4-(4,4,5,5-tetramethyl-1,3,2-dioxaborolan-2-yl)-1-((2-(trimethylsilyl)ethoxy)methyl)-1H-pyrrolo[2,3-b]pyridine (Intermediate 21) and also microwave irradiation at 100° C. for 1 h rather than conventional heating in Step A. MS (ESI): mass calcd. for C₁₈H₁₅FN₆ 334.1 m/z found 335.0 [M+H]⁺. ¹H NMR (400 MHz, DMSO-d₆) δ 13.49 (s, 1H), 8.40 (d, J=4.8 Hz, 1H), 8.28 (d, J=2.9 Hz, 1H), 7.86-7.73 (m, 2H), 7.39 (d, J=1.3 Hz, 1H), 7.01 (d, J=4.8 Hz, 1H), 4.55 (t, J=6.6 Hz, 1H), 3.08-2.89 (m, 2H), 2.87-2.76 (m, 1H), 2.22 (tdd, J=6.7, 8.8, 12.8 Hz, 1H), 1.53 (d, J=6.4 Hz, 3H).

### Example 21: 4-[(6R)-2-(5-Fluoro-2-pyridyl)-6-methyl-5,6-dihydro-4H-pyrrolo[1,2-b]pyrazol-3-yl]-6-methyl-1H-pyrazolo[3,4-b]pyridine

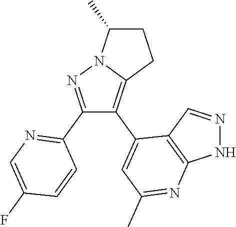 [SMILES: Cc1cc(-c2c(-c3ccc(F)cn3)nn3c2CC[C@H]3C)c2cn[nH]c2n1]

The title compound was prepared in a manner analogous to Example 1, Steps A-B, except using (R)-3-bromo-2-(5-fluoropyridin-2-yl)-6-methyl-5,6-dihydro-4H-pyrrolo[1,2-b]pyrazole (Intermediate 45) instead of 3-bromo-2-(5-fluoropyridin-2-yl)-6,7-dihydro-4H-pyrazolo[5,1-c][1,4]oxazine (Intermediate 37) and 6-methyl-4-(4,4,5,5-tetramethyl-1,3,2-dioxaborolan-2-yl)-1-((2-(trimethylsilyl)ethoxy)methyl)-1H-pyrazolo[3,4-b]pyridine (Intermediate 26) instead of 4-(4,4,5,5-tetramethyl-1,3,2-dioxaborolan-2-yl)-1-((2-(trimethylsilyl)ethoxy)methyl)-1H-pyrrolo[2,3-b]pyridine (Intermediate 21) and also microwave irradiation at 100° C. for 1 h rather than conventional heating in Step A. MS (ESI): mass calcd. for C₁₉H₁₇FN₆ 348.1 m/z, found 349.0 [M+H]⁺. ¹H NMR (400 MHz, DMSO-d₆) δ 13.25 (s, 1H), 8.28 (d, J=2.7 Hz, 1H), 7.87-7.69 (m, 2H), 7.22 (d, J=1.3 Hz, 1H), 6.93 (s, 1H), 4.54 (t, J=6.6 Hz, 1H), 3.09-2.88 (m, 2H), 2.81 (dtd, J=4.4, 8.1, 12.2 Hz, 1H), 2.53 (s, 3H), 2.21 (tdd, J=6.6, 8.8, 12.8 Hz, 1H), 1.53 (d, J=6.3 Hz, 3H).

### Example 22: 8-[(6S)-2-(5-Fluoro-2-pyridyl)-6-methyl-5,6-dihydro-4H-pyrrolo[1,2-b]pyrazol-3-yl]-2-methoxy-1,5-naphthyridine

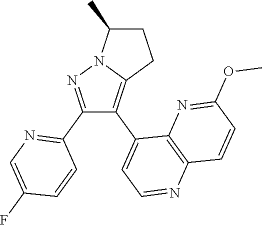 [SMILES: COc1ccc2nccc(-c3c(-c4ccc(F)cn4)nn4c3CC[C@@H]4C)c2n1]

The title compound was prepared in a manner analogous to Example 1, Step A, except using (6S)-3-bromo-2-(5-fluoro-2-pyridyl)-6-methyl-5,6-dihydro-4H-pyrrolo[1,2-b]pyrazole (Intermediate 44) instead of 3-bromo-2-(5-fluoropyridin-2-yl)-6,7-dihydro-4H-pyrazolo[5,1-c][1,4]oxazine (Intermediate 37) and 2-methoxy-8-(4,4,5,5-tetramethyl-1,3,2-dioxaborolan-2-yl)-1,5-naphthyridine (Intermediate 30) instead of 4-(4,4,5,5-tetramethyl-1,3,2-dioxaborolan-2-yl)-1-((2-(trimethylsilyl)ethoxy)methyl)-1H-pyrrolo[2,3-b]pyridine (Intermediate 21). MS (ESI): mass calcd. for C₂₁H₁₈FN₅O, 375.2; m/z found, 376.1 [M+H]⁺. ¹H NMR (400 MHz, CD₃OD) δ 8.65 (d, J=4.6 Hz, 1H), 8.17 (d, J=9.1 Hz, 1H), 8.12-8.03 (m, 1H), 7.70-7.61 (m, 1H), 7.59 (d, J=4.6 Hz, 1H), 7.54-7.41 (m, 1H), 7.10 (d, J=9.1 Hz, 1H), 4.60 (h, J=6.5 Hz, 1H), 3.58 (s, 3H), 3.18-2.82 (m, 3H), 2.44-2.20 (m, 1H), 1.63 (d, J=6.4 Hz, 3H).

### Example 23: (Racemic) 2-(5-Fluoropyridin-2-yl)-3-(1H-pyrazolo[3,4-b]pyridin-4-yl)-3b,4,4a,5-tetrahydrocyclopropa[3,4]pyrrolo[1,2-b]pyrazole

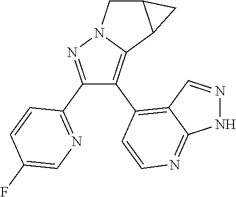 [SMILES: Fc1ccc(-c2nn3c(c2-c2ccnc4[nH]ncc24)C2CC2C3)nc1]

The title compound was prepared in a manner analogous to (4aR,5aR)-2-(5-fluoropyridin-2-yl)-3-(6-methyl-1H-pyrazolo[3,4-b]pyridin-4-yl)-4,4a,5,5a-tetrahydrocyclopropa[4,5]pyrrolo[1,2-b]pyrazole (Example 27) except using racemic (3bS,4aR)-3-bromo-2-(5-fluoropyridin-2-yl)-3b,4,4a,5-tetrahydrocyclopropa[3,4]pyrrolo[1,2-b]pyrazole (Intermediate 46) instead of (4aR,5aR)-3-Bromo-2-(5-fluoropyridin-2-yl)-4,4a,5,5a-tetrahydrocyclopropa[4,5]pyrrolo[1,2-b]pyrazole (Intermediate 47) and 4-(4,4,5,5-tetramethyl-1,3,2-dioxaborolan-2-yl)-1-((2-(trimethylsilyl)ethoxy)methyl)-1H-pyrazolo[3,4-b]pyridine (Intermediate 23) instead of 6-methyl-4-(4,4,5,5-tetramethyl-1,3,2-dioxaborolan-2-yl)-1-((2-(trimethylsilyl)ethoxy)methyl)-1H-pyrazolo[3,4-b]pyridine (Intermediate 26). MS (ESI): mass calcd. for C₁₈H₁₃FN₆, 332.1; m/z found, 333.1 [M+H]⁺. ¹H NMR (400 MHz, CDCl₃): δ 10.60 (s, 1H), 8.41 (d, J=4.8 Hz, 1H), 8.24 (d, J=2.9 Hz, 1H), 7.66 (d, J=0.8 Hz, 1H), 7.52-7.42 (m, 1H), 7.29-7.21 (m, 1H), 7.01 (d, J=4.8 Hz, 1H), 4.41 (dd, J=11.9, 5.6 Hz, 1H), 4.24 (d, J=12.0 Hz, 1H), 2.41-2.27 (m, 2H), 1.41-1.30 (m, 1H), 0.78 (q, J=4.5 Hz, 1H).

### Example 24: (Racemic) 2-(5-Fluoropyridin-2-yl)-3-(6-methyl-1H-pyrazolo[3,4-b]pyridin-4-yl)-3b,4,4a,5-tetrahydrocyclopropa[3,4]pyrrolo[1,2-b]pyrazole

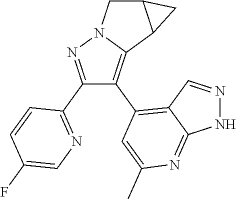 [SMILES: Cc1cc(-c2c(-c3ccc(F)cn3)nn3c2C2CC2C3)c2cn[nH]c2n1]

The title compound was prepared in a manner analogous to (4aR,5aR)-2-(5-fluoropyridin-2-yl)-3-(6-methyl-1H-pyrazolo[3,4-b]pyridin-4-yl)-4,4a,5,5a-tetrahydrocyclopropa[4,5]pyrrolo[1,2-b]pyrazole (Example 27), except using racemic (3bS,4aR)-3-bromo-2-(5-fluoropyridin-2-yl)-3b,4,4a,5-tetrahydrocyclopropa[3,4]pyrrolo[1,2-b]pyrazole (Intermediate 46) instead of (4aR,5aR)-3-bromo-2-(5-fluoropyridin-2-yl)-4,4a,5,5a-tetrahydrocyclopropa[4,5]pyrrolo[1,2-b]pyrazole (Intermediate 47). MS (ESI): mass calcd. for C₁₉H₁₅FN₆, 346.1; m/z found, 347.1 [M+H]⁺. ¹H NMR (400 MHz, CDCl₃): δ 10.77 (s, 1H), 8.24 (d, J=2.9 Hz, 1H), 7.53 (s, 1H), 7.52-7.39 (m, 1H), 7.31-7.21 (m, 1H), 6.91 (s, 1H), 4.41 (dd, J=11.9, 5.8 Hz, 1H), 4.23 (d, J=12.0 Hz, 1H), 2.59 (s, 3H), 2.44-2.26 (m, 2H), 1.41-1.30 (m, 1H), 0.77 (q, J=4.5 Hz, 1H).

### Example 25: (4aR,5aR)-2-(5-Fluoropyridin-2-yl)-3-(1H-pyrazolo[3,4-b]pyridin-4-yl)-4,4a,5,5a-tetrahydrocyclopropa[4,5]pyrrolo[1,2-b]pyrazole

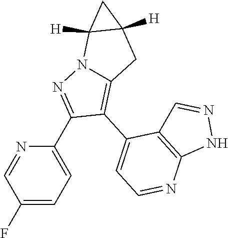 [SMILES: Fc1ccc(-c2nn3c(c2-c2ccnc4[nH]ncc24)C[C@H]2C[C@H]23)nc1]

The title compound was prepared in a manner analogous to (4aR,5aR)-2-(5-fluoropyridin-2-yl)-3-(6-methyl-1H-pyrazolo[3,4-b]pyridin-4-yl)-4,4a,5,5a-tetrahydrocyclopropa[4,5]pyrrolo[1,2-b]pyrazole (Example 27), except using 4-(4,4,5,5-tetramethyl-1,3,2-dioxaborolan-2-yl)-1-((2-(trimethylsilyl)ethoxy)methyl)-1H-pyrazolo[3,4-b]pyridine (Intermediate 23) instead of 6-methyl-4-(4,4,5,5-tetramethyl-1,3,2-dioxaborolan-2-yl)-1-((2-(trimethylsilyl)ethoxy)methyl)-1H-pyrazolo[3,4-b]pyridine (Intermediate 26). MS (ESI): mass calcd. for C₁₈H₁₃FN₆, 332.1; m/z found, 333.1 [M+H]⁺. ¹H NMR (500 MHz, CDCl₃): δ 10.83 (s, 1H), 8.42 (d, J=4.8 Hz, 1H), 8.18 (d, J=2.9 Hz, 1H), 7.62-7.52 (m, 1H), 7.37 (s, 1H), 7.29-7.23 (m, 1H), 6.94 (d, J=4.8 Hz, 1H), 4.13-4.05 (m, 1H), 3.24 (dd, J=17.1, 6.6 Hz, 1H), 3.02-2.85 (m, 1H), 2.31-2.18 (m, 1H), 1.24-1.12 (m, 1H), 0.66-0.49 (m, 1H).

### Example 26: (4aS,5aS)-2-(5-Fluoropyridin-2-yl)-3-(1H-pyrazolo[3,4-b]pyridin-4-yl)-4,4a,5,5a-tetrahydrocyclopropa[4,5]pyrrolo[1,2-b]pyrazole

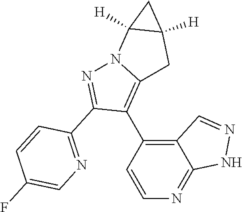 [SMILES: Fc1ccc(-c2nn3c(c2-c2ccnc4[nH]ncc24)C[C@@H]2C[C@@H]23)nc1]

The title compound was prepared in a manner analogous to (4aR,5aR)-2-(5-fluoropyridin-2-yl)-3-(6-methyl-1H-pyrazolo[3,4-b]pyridin-4-yl)-4,4a,5,5a-tetrahydrocyclopropa[4,5]pyrrolo[1,2-b]pyrazole (Example 27), except using (4aS,5aS)-3-bromo-2-(5-fluoropyridin-2-yl)-4,4a,5,5a-tetrahydrocyclopropa[4,5]pyrrolo[1,2-b]pyrazole (Intermediate 48) instead of (4aR,5aR)-3-bromo-2-(5-fluoropyridin-2-yl)-4,4a,5,5a-tetrahydrocyclopropa[4,5]pyrrolo[1,2-b]pyrazole (Intermediate 47) and 4-(4,4,5,5-tetramethyl-1,3,2-dioxaborolan-2-yl)-1-((2-(trimethylsilyl)ethoxy)methyl)-1H-pyrazolo[3,4-b]pyridine (Intermediate 23) instead of 6-methyl-4-(4,4,5,5-tetramethyl-1,3,2-dioxaborolan-2-yl)-1-((2-(trimethylsilyl)ethoxy)methyl)-1H-pyrazolo[3,4-b]pyridine (Intermediate 26). MS (ESI): mass calcd. for C₁₈H₁₃FN₆, 332.1; m/z found, 333.1 [M+H]⁺. ¹H NMR (500 MHz, CDCl₃): δ 10.71 (s, 1H), 8.42 (d, J=4.8 Hz, 1H), 8.18 (d, J=2.9 Hz, 1H), 7.64-7.47 (m, 1H), 7.37 (s, 1H), 7.31-7.21 (m, 1H), 6.94 (d, J=4.8 Hz, 1H), 4.13-4.03 (m, 1H), 3.30-3.16 (m, 1H), 3.00-2.82 (m, 1H), 2.27-2.16 (m, 1H), 1.28-1.09 (m, 1H), 0.67-0.53 (m, 1H).

### Example 27: (4aR,5aR)-2-(5-Fluoropyridin-2-yl)-3-(6-methyl-1H-pyrazolo[3,4-b]pyridin-4-yl)-4,4a,5,5a-tetrahydrocyclopropa[4,5]pyrrolo[1,2-b]pyrazole

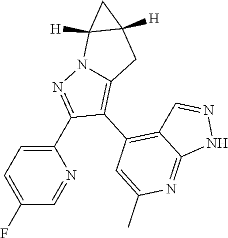 [SMILES: Cc1cc(-c2c(-c3ccc(F)cn3)nn3c2C[C@H]2C[C@H]23)c2cn[nH]c2n1]

Step A: (4aR,5aR)-2-(5-Fluoropyridin-2-yl)-3-(6-methyl-1-((2-(trimethylsilyl)ethoxy)methyl)-1H-pyrazolo[3,4-b]pyridin-4-yl)-4,4a,5,5a-tetrahydrocyclopropa[4,5]pyrrolo[1,2-b]pyrazole. (4aR,5aR)-3-Bromo-2-(5-fluoropyridin-2-yl)-4,4a,5,5a-tetrahydrocyclopropa[4,5]pyrrolo[1,2-b]pyrazole (Intermediate 47, 35 mg, 0.119 mmol), 6-methyl-4-(4,4,5,5-tetramethyl-1,3,2-dioxaborolan-2-yl)-1-((2-(trimethylsilyl)ethoxy)methyl)-1H-pyrazolo[3,4-b]pyridine (Intermediate 26, 60.2 mg, 0.155 mmol), CataXcium Pd G4 (4.3 mg, 0.006 mmol), and Cs₂CO₃ (117 mg, 0.36 mmol) were dissolved in a mixture of 2-methyl-2-butanol (2 mL) and water (1 mL). The biphasic mixture was stirred at 90° C. for 2 hours and then partitioned between water and DCM. The aqueous layer was extracted 2×DCM, and the combined organics were concentrated and purified on silica gel (0-100% ethyl acetate/hexanes) to obtain 21.0 mg (37% yield) of the title compound. MS (ESI): mass calcd. for C₂₅H₂₉FN₆OSi, 476.2; m/z found, 477.3 [M+H]⁺.

Step B: (4aR,5aR)-2-(5-Fluoropyridin-2-yl)-3-(6-methyl-1H-pyrazolo[3,4-b]pyridin-4-yl)-4,4a,5,5a-tetrahydrocyclopropa[4,5]pyrrolo[1,2-b]pyrazole. (4aR,5aR)-2-(5-Fluoropyridin-2-yl)-3-(6-methyl-1-((2-(trimethylsilyl)ethoxy)methyl)-1H-pyrazolo[3,4-b]pyridin-4-yl)-4,4a,5,5a-tetrahydrocyclopropa[4,5]pyrrolo[1,2-b]pyrazole (21.0 mg, 0.0441 mmol) was dissolved in TFA (1 mL) and stirred at r.t. for two hours. The reaction mixture was concentrated under reduced pressure. The resulting residue was purified by HPLC Method D: to obtain 8.1 mg (53% yield) of the title compound. MS (ESI): mass calcd. for C₁₉H₁₅FN₆, 346.1; m/z found, 347.1 [M+H]⁺. ¹H NMR (500 MHz, CDCl₃): δ 8.10 (d, J=2.8 Hz, 1H), 7.77-7.64 (m, 1H), 7.38-7.29 (m, 2H), 6.91 (s, 1H), 4.16-4.07 (m, 1H), 3.26 (dd, J=17.2, 6.7 Hz, 1H), 2.95 (d, J=16.9 Hz, 1H), 2.71 (s, 3H), 2.34-2.18 (m, 1H), 1.25-1.14 (m, 1H), 0.69-0.53 (m, 1H). The product contains an exchangeable N—H proton which was not observed by ¹H NMR.

### Example 28: (4aS,5aS)-2-(5-Fluoropyridin-2-yl)-3-(6-methyl-1H-pyrazolo[3,4-b]pyridin-4-yl)-4,4a,5,5a-tetrahydrocyclopropa[4,5]pyrrolo[1,2-b]pyrazole

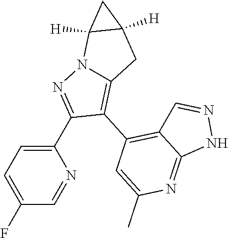 [SMILES: Cc1cc(-c2c(-c3ccc(F)cn3)nn3c2C[C@@H]2C[C@@H]23)c2cn[nH]c2n1]

The title compound was prepared in a manner analogous to (4aR,5aR)-2-(5-fluoropyridin-2-yl)-3-(6-methyl-1H-pyrazolo[3,4-b]pyridin-4-yl)-4,4a,5,5a-tetrahydrocyclopropa[4,5]pyrrolo[1,2-b]pyrazole (Example 27), except using (4aS,5aS)-3-bromo-2-(5-fluoropyridin-2-yl)-4,4a,5,5a-tetrahydrocyclopropa[4,5]pyrrolo[1,2-b]pyrazole (Intermediate 48) instead of (4aR,5aR)-3-bromo-2-(5-fluoropyridin-2-yl)-4,4a,5,5a-tetrahydrocyclopropa[4,5]pyrrolo[1,2-b]pyrazole (Intermediate 47). MS (ESI): mass calcd. for C₁₉H₁₅FN₆, 346.1; m/z found, 347.1 [M+H]⁺. ¹H NMR (500 MHz, CDCl₃): δ 11.15 (s, 1H), 8.27 (d, J=2.9 Hz, 1H), 7.65-7.50 (m, 1H), 7.39-7.30 (m, 2H), 6.91 (s, 1H), 4.22-4.10 (m, 1H), 3.32 (dd, J=17.0, 6.6 Hz, 1H), 3.01 (dd, J=17.3, 1.3 Hz, 1H), 2.68 (s, 3H), 2.39-2.21 (m, 1H), 1.33-1.16 (m, 1H), 0.73-0.55 (m, 1H).

### Example 29: 2-(3-(1H-Pyrazolo[3,4-b]pyridin-4-yl)-4,5,6,7-tetrahydropyrazolo[1,5-a]pyridin-2-yl)thiazole

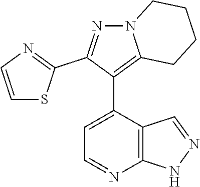 [SMILES: c1csc(-c2nn3c(c2-c2ccnc4[nH]ncc24)CCCC3)n1]

The title compound was prepared in a manner analogous to Example 1, Steps A-B, except using 2-(3-bromo-4,5,6,7-tetrahydropyrazolo[1,5-a]pyridin-2-yl)thiazole (Intermediate 147) instead of 3-bromo-2-(5-fluoropyridin-2-yl)-6,7-dihydro-4H-pyrazolo[5,1-c][1,4]oxazine (Intermediate 37) and 4-(4,4,5,5-tetramethyl-1,3,2-dioxaborolan-2-yl)-1-((2-(trimethylsilyl)ethoxy)methyl)-1H-pyrazolo[3,4-b]pyridine (Intermediate 23) instead of 4-(4,4,5,5-tetramethyl-1,3,2-dioxaborolan-2-yl)-1-((2-(trimethylsilyl)ethoxy)methyl)-1H-pyrrolo[2,3-b]pyridine (Intermediate 21) in Step A. MS (ESI): mass calcd. for C₁₆H₁₄N₆S, 322.1; m/z found, 323.0 [M+H]⁺. ¹H NMR (500 MHz, CD₃OD) δ 8.55 (d, J=4.8 Hz, 1H), 7.62 (d, J=3.3 Hz, 1H), 7.57-7.53 (m, 2H), 7.22 (d, J=4.8 Hz, 1H), 4.33 (t, J=6.2 Hz, 2H), 2.86 (t, J=6.4 Hz, 2H), 2.25-2.18 (m, 2H), 2.00-1.93 (m, 2H).

### Example 30: 2-(3-(6-Methyl-1H-pyrazolo[3,4-b]pyridin-4-yl)-4,5,6,7-tetrahydropyrazolo[1,5-a]pyridin-2-yl)thiazole

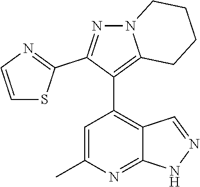 [SMILES: Cc1cc(-c2c(-c3nccs3)nn3c2CCCC3)c2cn[nH]c2n1]

The title compound was prepared in a manner analogous to Example 1, Steps A-B, except using 2-(3-bromo-4,5,6,7-tetrahydropyrazolo[1,5-a]pyridin-2-yl)thiazole (Intermediate 147) instead of 3-bromo-2-(5-fluoropyridin-2-yl)-6,7-dihydro-4H-pyrazolo[5,1-c][1,4]oxazine (Intermediate 37) and 6-methyl-4-(4,4,5,5-tetramethyl-1,3,2-dioxaborolan-2-yl)-1-((2-(trimethylsilyl)ethoxy)methyl)-1H-pyrazolo[3,4-b]pyridine (Intermediate 26) instead of 4-(4,4,5,5-tetramethyl-1,3,2-dioxaborolan-2-yl)-1-((2-(trimethylsilyl)ethoxy)methyl)-1H-pyrrolo[2,3-b]pyridine (Intermediate 21) in Step A. MS (ESI): mass calcd. for C₁₇H₁₆N₆S, 336.1; m/z found, 337.0 [M+H]⁺. ¹H NMR (500 MHz, CD₃OD) δ 8.01 (s, 1H), 7.63 (d, J=3.3 Hz, 1H), 7.54 (d, J=3.3 Hz, 1H), 7.46 (s, 1H), 7.11 (s, 1H), 4.32 (t, J=6.2 Hz, 2H), 2.85 (t, J=6.4 Hz, 2H), 2.70 (s, 3H), 2.21 (td, J=8.0, 7.0, 4.2 Hz, 2H), 2.00-1.92 (m, 2H).

### Example 31: 4-(3-(1H-Pyrazolo[3,4-b]pyridin-4-yl)-4,5,6,7-tetrahydropyrazolo[1,5-a]pyridin-2-yl)thiazole

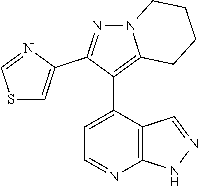 [SMILES: c1cc(-c2c(-c3cscn3)nn3c2CCCC3)c2cn[nH]c2n1]

The title compound was prepared in a manner analogous to Example 1, Steps A-B, except using 4-(3-bromo-4,5,6,7-tetrahydropyrazolo[1,5-a]pyridin-2-yl)thiazole (Intermediate 102) instead of 3-bromo-2-(5-fluoropyridin-2-yl)-6,7-dihydro-4H-pyrazolo[5,1-c][1,4]oxazine (Intermediate 37) and 4-(4,4,5,5-tetramethyl-1,3,2-dioxaborolan-2-yl)-1-((2-(trimethylsilyl)ethoxy)methyl)-1H-pyrazolo[3,4-b]pyridine (Intermediate 23) instead of 4-(4,4,5,5-tetramethyl-1,3,2-dioxaborolan-2-yl)-1-((2-(trimethylsilyl)ethoxy)methyl)-1H-pyrrolo[2,3-b]pyridine (Intermediate 21) in Step A. MS (ESI): mass calcd. for C₁₆H₁₄N₆S, 322.1; m/z found, 323.1 [M+H]⁺. ¹H NMR (500 MHz, CD₃OD) δ 8.88 (d, J=2.0 Hz, 1H), 8.53 (d, J=4.8 Hz, 1H), 7.55 (d, J=2.0 Hz, 1H), 7.47 (s, 1H), 7.17 (d, J=4.8 Hz, 1H), 4.32 (t, J=6.2 Hz, 2H), 2.89 (t, J=6.3 Hz, 2H), 2.26-2.17 (m, 2H), 2.00-1.92 (m, 2H).

### Example 32: 4-(3-(6-Methyl-1H-pyrazolo[3,4-b]pyridin-4-yl)-4,5,6,7-tetrahydropyrazolo[1,5-a]pyridin-2-yl)thiazole

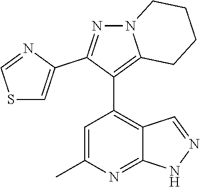 [SMILES: Cc1cc(-c2c(-c3cscn3)nn3c2CCCC3)c2cn[nH]c2n1]

The title compound was prepared in a manner analogous to Example 1, Steps A-B, except using 4-(3-bromo-4,5,6,7-tetrahydropyrazolo[1,5-a]pyridin-2-yl)thiazole (Intermediate 102) instead of 3-bromo-2-(5-fluoropyridin-2-yl)-6,7-dihydro-4H-pyrazolo[5,1-c][1,4]oxazine (Intermediate 37) and 6-methyl-4-(4,4,5,5-tetramethyl-1,3,2-dioxaborolan-2-yl)-1-((2-(trimethylsilyl)ethoxy)methyl)-1H-pyrazolo[3,4-b]pyridine (Intermediate 26) instead of 4-(4,4,5,5-tetramethyl-1,3,2-dioxaborolan-2-yl)-1-((2-(trimethylsilyl)ethoxy)methyl)-1H-pyrrolo[2,3-b]pyridine (Intermediate 21) in Step A. MS (ESI): mass calcd. for C₁₇H₁₆N₆S, 336.1; m/z found, 337.0 [M+H]⁺. ¹H NMR (400 MHz, CD₃OD) δ 8.87 (d, J=2.0 Hz, 1H), 7.52 (d, J=2.0 Hz, 1H), 7.36 (s, 1H), 7.06 (s, 1H), 4.31 (t, J=6.2 Hz, 2H), 2.88 (t, J=6.3 Hz, 2H), 2.68 (s, 3H), 2.26-2.16 (m, 2H), 2.02-1.90 (m, 2H).

### Example 33: 4-[2-(4-Fluorophenyl)-4,5,6,7-tetrahydropyrazolo[1,5-a]pyridin-3-yl]-1H-pyrrolo[2,3-b]pyridine

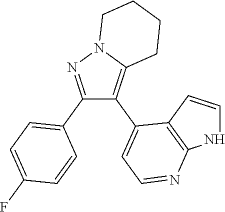 [SMILES: Fc1ccc(-c2nn3c(c2-c2ccnc4[nH]ccc24)CCCC3)cc1]

In a pressure vial was placed 2-(4-fluorophenyl)-3-iodo-4,5,6,7-tetrahydropyrazolo[1,5-a]pyridine (Intermediate 140, 110 mg, 0.321 mmol), tert-butyl 4-(4,4,5,5-tetramethyl-1,3,2-dioxaborolan-2-yl)-1H-pyrrolo[2,3-b]pyridine-1-carboxylate (Intermediate 139, 165 mg, 0.479 mmol), Pd(dppf)Cl₂ and 2M Na₂CO₃ (0.321 mL, 0.642 mmol). The reagents were brought up in 1,4-dioxane (3.5 mL) and the vial was sealed. The resultant mixture was heated at 130° C. via microwave irradiation for 3 h. The reaction mixture was cooled to room temperature. The resultant mixture was diluted with H₂O, the suspension was filtered through a pad of Celite® and the pad washed with ethyl acetate (2×20 mL). The filtrate layers were separated and the aqueous phase was extracted with EtOAc (40 mL). The combined organics were washed with brine, dried over MgSO₄, filtered and evaporated. The resulting residue was purified by FCC (SiO₂, 2:1 then 1:4, hexanes:EtOAc) followed by trituration with diisopropyl ether afforded the title compound (25 mg, 23%). MS (ESI): mass calcd. for C₂₀H₁₇FN₄, 332.1; found 333.2 [M+H]⁺. ¹H NMR (300 MHz, DMSO-d₆) δ 11.60 (s, 1H), 8.18 (d, J=4.9 Hz, 1H), 7.39-7.23 (m, 3H), 7.10-6.96 (m, 2H), 6.88 (d, J=4.9 Hz, 1H), 5.91 (dd, J=3.5, 1.9 Hz, 1H), 4.27-4.11 (m, 2H), 2.69-2.59 (m, 2H), 2.13-1.96 (m, 2H), 1.89-1.70 (m, 2H).

### Example 34: 5-Fluoro-4-[2-(5-fluoro-2-pyridyl)-4,5,6,7-tetrahydropyrazolo[1,5-a]pyridin-3-yl]-1H-pyrrolo[2,3-b]pyridine

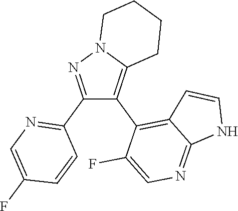 [SMILES: Fc1ccc(-c2nn3c(c2-c2c(F)cnc4[nH]ccc24)CCCC3)nc1]

The title compound was prepared in a manner analogous to Example 1, Step A, using 5-fluoro-4-(4,4,5,5-tetramethyl-1,3,2-dioxaborolan-2-yl)-1-(triisopropylsilyl)-1H-pyrrolo[2,3-b]pyridine instead of 4-(4,4,5,5-tetramethyl-1,3,2-dioxaborolan-2-yl)-1-((2-(trimethylsilyl)ethoxy)methyl)-1H-pyrrolo[2,3-b]pyridine (Intermediate 21) and 3-bromo-2-(5-fluoropyridin-2-yl)-4,5,6,7-tetrahydropyrazolo[1,5-a]pyridine (Intermediate 49) instead of 3-bromo-2-(5-fluoropyridin-2-yl)-6,7-dihydro-4H-pyrazolo[5,1-c][1,4]oxazine (Intermediate 37). MS (ESI): mass calcd. for C₁₉H₁₅F₂N₅, 351.1; m/z found, 352.1 [M+H]⁺. ¹H NMR (500 MHz, CDCl₃): δ 9.96 (s, 1H), 8.30 (d, J=2.9 Hz, 1H), 8.23 (s, 1H), 7.41 (ddd, J=8.8, 4.5, 0.6 Hz, 1H), 7.25-7.24 (m, 1H), 7.20 (ddd, J=8.8, 8.1, 2.9 Hz, 1H), 6.05 (dd, J=3.5, 1.1 Hz, 1H), 4.41-4.27 (m, 2H), 2.79-2.59 (m, 2H), 2.20-2.11 (m, 2H), 1.98-1.79 (m, 2H).

### Example 35: 4-[2-(5-Fluoro-2-pyridyl)-4,5,6,7-tetrahydropyrazolo[1,5-a]pyridin-3-yl]-1H-pyrazolo[3,4-b]pyridine

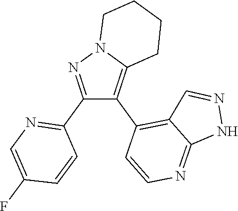 [SMILES: Fc1ccc(-c2nn3c(c2-c2ccnc4[nH]ncc24)CCCC3)nc1]

The title compound was prepared in a manner analogous to Example 1, Steps A-B, except using 4-(4,4,5,5-tetramethyl-1,3,2-dioxaborolan-2-yl)-1-((2-(trimethylsilyl)ethoxy)methyl)-1H-pyrazolo[3,4-b]pyridine (Intermediate 23) instead of 4-(4,4,5,5-tetramethyl-1,3,2-dioxaborolan-2-yl)-1-((2-(trimethylsilyl)ethoxy)methyl)-1H-pyrrolo[2,3-b]pyridine (Intermediate 21) and 3-bromo-2-(5-fluoropyridin-2-yl)-4,5,6,7-tetrahydropyrazolo[1,5-a]pyridine (Intermediate 49) instead of 3-bromo-2-(5-fluoropyridin-2-yl)-6,7-dihydro-4H-pyrazolo[5,1-c][1,4]oxazine (Intermediate 37) in Step A. MS (ESI): mass calcd. for C₁₈H₁₅FN₆, 334.1; m/z found, 335.1 [M+H]⁺. ¹H NMR (500 MHz, CDCl₃): δ 8.59 (d, J=4.8 Hz, 1H), 8.28 (d, J=2.9 Hz, 1H), 7.52 (s, 1H), 7.47 (dd, J=8.7, 4.4 Hz, 1H), 7.29-7.22 (m, 2H), 7.06 (d, J=4.9 Hz, 1H), 4.34 (t, J=6.2 Hz, 2H), 2.81 (t, J=6.3 Hz, 2H), 2.20-2.12 (m, 2H), 1.96-1.86 (m, 2H).

### Example 36: 4-[2-(5-Fluoro-2-pyridyl)-4,5,6,7-tetrahydropyrazolo[1,5-a]pyridin-3-yl]-6-methyl-1H-pyrazolo[3,4-b]pyridine

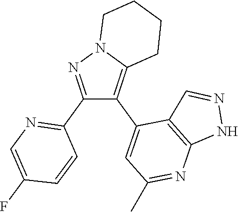 [SMILES: Cc1cc(-c2c(-c3ccc(F)cn3)nn3c2CCCC3)c2cn[nH]c2n1]

The title compound was prepared in a manner analogous to Example 1, Steps A-B, using 6-methyl-4-(4,4,5,5-tetramethyl-1,3,2-dioxaborolan-2-yl)-1-((2-(trimethylsilyl)ethoxy)methyl)-1H-pyrazolo[3,4-b]pyridine (Intermediate 26) instead of 4-(4,4,5,5-tetramethyl-1,3,2-dioxaborolan-2-yl)-1-((2-(trimethylsilyl)ethoxy)methyl)-1H-pyrrolo[2,3-b]pyridine (Intermediate 21) and 3-bromo-2-(5-fluoropyridin-2-yl)-4,5,6,7-tetrahydropyrazolo[1,5-a]pyridine (Intermediate 49) instead of 3-bromo-2-(5-fluoropyridin-2-yl)-6,7-dihydro-4H-pyrazolo[5,1-c][1,4]oxazine (Intermediate 37) in Step A. MS (ESI): mass calcd. for C₁₉H₁₇FN₆, 348.1; m/z found, 349.1 [M+H]⁺. ¹H NMR (400 MHz, CDCl₃): δ 12.98 (s, 1H), 8.30 (d, J=2.9 Hz, 1H), 7.44 (dd, J=8.7, 4.4 Hz, 1H), 7.40 (s, 1H), 7.28-7.22 (m, 1H), 6.94 (s, 1H), 4.33 (t, J=6.2 Hz, 2H), 2.82 (t, J=6.3 Hz, 2H), 2.77 (s, 3H), 2.20-2.11 (m, 2H), 1.96-1.86 (m, 2H).

### Example 37: 4-[2-(3,5-Difluoro-2-pyridyl)-4,5,6,7-tetrahydropyrazolo[1,5-a]pyridin-3-yl]-6-methyl-1H-pyrazolo[3,4-b]pyridine

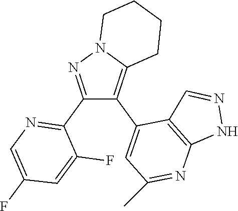 [SMILES: Cc1cc(-c2c(-c3ncc(F)cc3F)nn3c2CCCC3)c2cn[nH]c2n1]

Step A: 4-(2-(3,5-Difluoropyridin-2-yl)-4,5,6,7-tetrahydropyrazolo[1,5-a]pyridin-3-yl)-6-methyl-1-((2-(trimethylsilyl)ethoxy)methyl)-1H-pyrazolo[3,4-b]pyridine. The title compound was prepared in a manner analogous to Example 1, Step A, using 6-methyl-4-(4,4,5,5-tetramethyl-1,3,2-dioxaborolan-2-yl)-1-((2-(trimethylsilyl)ethoxy)methyl)-1H-pyrazolo[3,4-b]pyridine (Intermediate 26) instead of 4-(4,4,5,5-tetramethyl-1,3,2-dioxaborolan-2-yl)-1-((2-(trimethylsilyl)ethoxy)methyl)-1H-pyrrolo[2,3-b]pyridine (Intermediate 21) and 3-bromo-2-(3,5-difluoropyridin-2-yl)-4,5,6,7-tetrahydropyrazolo[1,5-a]pyridine (Intermediate 50) instead of 3-bromo-2-(5-fluoropyridin-2-yl)-6,7-dihydro-4H-pyrazolo[5,1-c][1,4]oxazine (Intermediate 37). MS (ESI): mass calcd. for C₂₅H₃₀F₂N₆OSi, 496.2; m/z found, 497.2 [M+H]⁺.

Step B: 4-[2-(3,5-Difluoro-2-pyridyl)-4,5,6,7-tetrahydropyrazolo[1,5-a]pyridin-3-yl]-6-methyl-1H-pyrazolo[3,4-b]pyridine. To 4-(2-(3,5-difluoropyridin-2-yl)-4,5,6,7-tetrahydropyrazolo[1,5-a]pyridin-3-yl)-6-methyl-1-((2-(trimethylsilyl)ethoxy)methyl)-1H-pyrazolo[3,4-b]pyridine in 1,4-dioxane (1.6 mL) was added 4M HCl in 1,4-dioxane (0.8 mL, 3.2 mmol) and reaction mixture was stirred at room temperature for 16 hours. More 4M HCl in 1,4-dioxane (0.8 mL, 3.2 mmol) was added and the reaction mixture was stirred at room temperature for 3 hours. The reaction mixture was concentrated under reduced pressure. Purification by basic prep HPLC Method C: gave the title compound (29 mg, 50%). MS (ESI): mass calcd. for C₁₉H₁₆F₂N₆, 366.1; m/z found, 367.1 [M+H]⁺. ¹H NMR (400 MHz, DMSO-d₆) δ 13.28 (s, 1H), 8.40 (d, J=2.4 Hz, 1H), 7.90 (ddd, J=10.0, 8.9, 2.4 Hz, 1H), 7.19 (d, J=1.0 Hz, 1H), 6.88 (s, 1H), 4.24 (t, J=6.1 Hz, 2H), 2.86 (t, J=6.2 Hz, 2H), 2.53 (s, 3H), 2.13-2.04 (m, 2H), 1.91-1.80 (m, 2H).

### Example 38: 4-[2-(3,5-Difluoro-2-pyridyl)-4,5,6,7-tetrahydropyrazolo[1,5-a]pyridin-3-yl]-1H-pyrazolo[3,4-b]pyridine

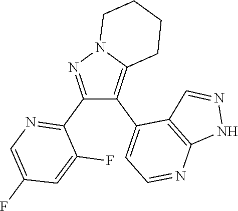 [SMILES: Fc1cnc(-c2nn3c(c2-c2ccnc4[nH]ncc24)CCCC3)c(F)c1]

The title compound was prepared in a manner analogous to Example 37, Steps A-B, except using 4-(4,4,5,5-tetramethyl-1,3,2-dioxaborolan-2-yl)-1-((2-(trimethylsilyl)ethoxy)methyl)-1H-pyrazolo[3,4-b]pyridine (Intermediate 23) instead of 6-methyl-4-(4,4,5,5-tetramethyl-1,3,2-dioxaborolan-2-yl)-1-((2-(trimethylsilyl)ethoxy)methyl)-1H-pyrazolo[3,4-b]pyridine (Intermediate 26) in Step A. MS (ESI): mass calcd. for C₁₈H₁₄F₂N₆, 352.1; m/z found, 353.1 [M+H]⁺. ¹H NMR (400 MHz, DMSO-d₆) δ 13.53 (s, 1H), 8.46-8.36 (m, 2H), 7.90 (ddd, J=10.0, 8.9, 2.4 Hz, 1H), 7.36 (s, 1H), 6.95 (d, J=4.8 Hz, 1H), 4.25 (t, J=6.1 Hz, 2H), 2.85 (t, J=6.2 Hz, 2H), 2.14-2.04 (m, 2H), 1.91-1.79 (m, 2H).

### Example 39: (*R)-4-[2-(4-Fluorophenyl)-7-methyl-4,5,6,7-tetrahydropyrazolo[1,5-a]pyridin-3-yl]-1H-pyrazolo[3,4-b]pyridine

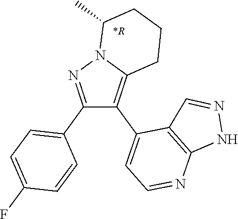 [SMILES: C[C@@H]1CCCc2c(-c3ccnc4[nH]ncc34)c(-c3ccc(F)cc3)nn21]

Step A: Racemic-4-(2-(4-Fluorophenyl)-7-methyl-4,5,6,7-tetrahydropyrazolo[1,5-a]pyridin-3-vi)-1H-pyrazolo[3,4-b]pyridine. The title compound was prepared in a manner analogous to Example 1, Steps A-B, except using 4-(4,4,5,5-tetramethyl-1,3,2-dioxaborolan-2-yl)-1-((2-(trimethylsilyl)ethoxy)methyl)-1H-pyrazolo[3,4-b]pyridine (Intermediate 23) instead of 4-(4,4,5,5-tetramethyl-1,3,2-dioxaborolan-2-yl)-1-((2-(trimethylsilyl)ethoxy)methyl)-1H-pyrrolo[2,3-b]pyridine (Intermediate 21) and 3-bromo-2-(4-fluorophenyl)-7-methyl-4,5,6,7-tetrahydropyrazolo[1,5-a]pyridine (Intermediate 51) instead of 3-bromo-2-(5-fluoropyridin-2-yl)-6,7-dihydro-4H-pyrazolo[5,1-c][1,4]oxazine (Intermediate 37) and Pd(dppf)Cl₂ instead of CataCXium® A Pd G3 in Step A.

Step B: (*R)-4-[2-(4-Fluorophenyl)-7-methyl-4,5,6,7-tetrahydropyrazolo[1,5-a]pyridin-3-yl]-1H-pyrazolo[3,4-b]pyridine. Racemic-4-(2-(4-fluorophenyl)-7-methyl-4,5,6,7-tetrahydropyrazolo[1,5-a]pyridin-3-yl)-1H-pyrazolo[3,4-b]pyridine was purified by SFC Method F. The pure fractions were collected, and the volatiles were removed under reduced pressure. The product was suspended in water (10 mL), the mixture frozen using dry ice/EtOH, and then lyophilized to dryness to afford (*R)-4-[2-(4-fluorophenyl)-7-methyl-4,5,6,7-tetrahydropyrazolo[1,5-a]pyridin-3-yl]-1H-pyrazolo[3,4-b]pyridine as a white solid (58.0 mg, 19%) as a white solid. MS (ESI): mass calcd. for C₂₀H₁₈FN₅ 347.2 m/z found 348.0 [M+H]⁺. ¹H NMR (400 MHz, DMSO-d₆) δ 13.55 (br s, 1H), 8.47 (d, J=4.6 Hz, 1H), 7.43 (s, 1H), 7.31-7.25 (m, 2H), 7.12-7.03 (m, 2H), 7.01 (d, J=4.6 Hz, 1H), 4.41-4.28 (m, 1H), 2.85-2.74 (m, 1H), 2.73-2.62 (m, 1H), 2.26-2.15 (m, 1H), 1.96-1.85 (m, 1H), 1.84-1.65 (m, 2H), 1.58 (d, J=6.4 Hz, 3H); and (*S)-4-[2-(4-Fluorophenyl)-7-methyl-4,5,6,7-tetrahydropyrazolo[1,5-a]pyridin-3-yl]-1H-pyrazolo[3,4-b]pyridine (Example 40, 41.9 mg, 14%)

### Example 40: (*S)-4-[2-(4-Fluorophenyl)-7-methyl-4,5,6,7-tetrahydropyrazolo[1,5-a]pyridin-3-yl]-1H-pyrazolo[3,4-b]pyridine

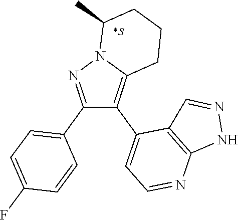 [SMILES: C[C@H]1CCCc2c(-c3ccnc4[nH]ncc34)c(-c3ccc(F)cc3)nn21]

The title compound was isolated from Example 39, Step B. MS (ESI): mass calcd. for C₂₀H₁₈FN₅ 347.2 m/z found 348.0 [M+H]⁺. ¹H NMR (400 MHz, DMSO-d₆) δ 13.56 (br s, 1H), 8.47 (d, J=4.8 Hz, 1H), 7.43 (s, 1H), 7.32-7.24 (m, 2H), 7.13-7.04 (m, 2H), 7.01 (d, J=4.8 Hz, 1H), 4.42-4.27 (m, 1H), 2.86-2.73 (m, 1H), 2.72-2.62 (m, 1H), 2.27-2.15 (m, 1H), 1.97-1.84 (m, 1H), 1.84-1.65 (m, 2H), 1.59 (d, J=6.4 Hz, 3H).

### Example 41: 4-(6,6-Difluoro-2-(4-fluorophenyl)-4,5,6,7-tetrahydropyrazolo[1,5-a]pyridin-3-yl)pyridin-2-amine

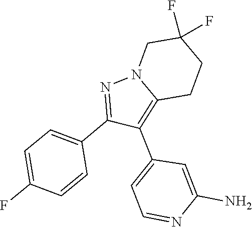 [SMILES: Nc1cc(-c2c(-c3ccc(F)cc3)nn3c2CCC(F)(F)C3)ccn1]

3-Bromo-6,6-difluoro-2-(4-fluorophenyl)-4,5,6,7-tetrahydropyrazolo[1,5-a]pyridine (Intermediate 52, 35 mg, 0.106 mmol), 4-(4,4,5,5-tetramethyl-1,3,2-dioxaborolan-2-yl)pyridin-2-amine (30.2 mg, 0.137 mmol), CataXcium Pd G4 (3.9 mg, 0.0053 mmol), and Cs₂CO₃ (104 mg, 0.317 mmol) were dissolved in a mixture of 2-methyl-2-butanol (2 mL) and water (1 mL). The biphasic mixture was stirred at 90° C. for 2 hours, at which point another 60 mg (0.28 mmol) 4-(4,4,5,5-tetramethyl-1,3,2-dioxaborolan-2-yl)pyridin-2-amine and 8 mg (0.01 mmol) of CataXcium Pd G4 were added and the mixture was stirred overnight at 90° C. The reaction mixture was partitioned between DCM and water, the aqueous layer was extracted 2× with DCM, the combined organics were concentrated and purified by reverse-phase HPLC Method D: to obtain 7.5 mg (0.022 mmol, 21% yield) of the title compound. MS (ESI): mass calcd. for C₁₈H₁₅F₃N₄, 344.1; m/z found, 345.2 [M+H]⁺. ¹H NMR (400 MHz, CDCl₃): δ 7.95 (dd, J=5.3, 0.8 Hz, 1H), 7.38-7.29 (m, 2H), 6.99-6.83 (m, 2H), 6.38 (dd, J=5.3, 1.4 Hz, 1H), 6.24-6.12 (m, 1H), 4.46 (t, J=12.3 Hz, 2H), 4.30 (s, 2H), 2.94 (t, J=6.8 Hz, 2H), 2.36-2.20 (m, 2H).

### Example 42: 4-(6,6-Difluoro-2-(4-fluorophenyl)-4,5,6,7-tetrahydropyrazolo[1,5-a]pyridin-3-yl)-1H-pyrazolo[3,4-b]pyridine

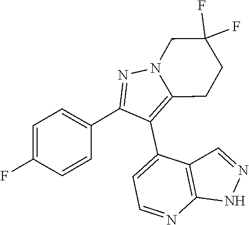 [SMILES: Fc1ccc(-c2nn3c(c2-c2ccnc4[nH]ncc24)CCC(F)(F)C3)cc1]

The title compound was prepared in a manner analogous to (4aR,5aR)-2-(5-fluoropyridin-2-yl)-3-(6-methyl-1H-pyrazolo[3,4-b]pyridin-4-yl)-4,4a,5,5a-tetrahydrocyclopropa[4,5]pyrrolo[1,2-b]pyrazole (Example 27), except using 3-bromo-6,6-difluoro-2-(4-fluorophenyl)-4,5,6,7-tetrahydropyrazolo[1,5-a]pyridine (Intermediate 52) instead of (4aR,5aR)-3-Bromo-2-(5-fluoropyridin-2-yl)-4,4a,5,5a-tetrahydrocyclopropa[4,5]pyrrolo[1,2-b]pyrazole (Intermediate 47) and 4-(4,4,5,5-tetramethyl-1,3,2-dioxaborolan-2-yl)-1-((2-(trimethylsilyl)ethoxy)methyl)-1H-pyrazolo[3,4-b]pyridine (Intermediate 23) instead of 6-methyl-4-(4,4,5,5-tetramethyl-1,3,2-dioxaborolan-2-yl)-1-((2-(trimethylsilyl)ethoxy)methyl)-1H-pyrazolo[3,4-b]pyridine (Intermediate 26). MS (ESI): mass calcd. for C₁₉H₁₄F₃N₅, 369.1; m/z found, 370.1 [M+H]⁺. ¹H NMR (400 MHz, CDCl₃): δ 10.77 (s, 1H), 8.48 (d, J=4.8 Hz, 1H), 7.44 (s, 1H), 7.29-7.22 (m, 2H), 6.92 (d, J=4.7 Hz, 1H), 6.87-6.80 (m, 2H), 4.54 (t, J=12.3 Hz, 2H), 2.93 (t, J=6.7 Hz, 2H), 2.40-2.21 (m, 2H).

### Example 43: 4-[6,6-Difluoro-2-(4-fluorophenyl)-5,7-dihydro-4H-pyrazolo[1,5-a]pyridin-3-yl]-6-methyl-1H-pyrazolo[3,4-b]pyridine

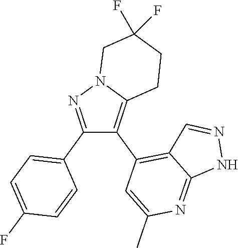 [SMILES: Cc1cc(-c2c(-c3ccc(F)cc3)nn3c2CCC(F)(F)C3)c2cn[nH]c2n1]

The title compound was prepared in a manner analogous to Example 1, Steps A-B, except using 6-methyl-4-(4,4,5,5-tetramethyl-1,3,2-dioxaborolan-2-yl)-1-((2-(trimethylsilyl)ethoxy)methyl)-1H-pyrazolo[3,4-b]pyridine (Intermediate 26) instead of 4-(4,4,5,5-tetramethyl-1,3,2-dioxaborolan-2-yl)-1-((2-(trimethylsilyl)ethoxy)methyl)-1H-pyrrolo[2,3-b]pyridine (Intermediate 21) and 3-bromo-6,6-difluoro-2-(4-fluorophenyl)-4,5,6,7-tetrahydropyrazolo[1,5-a]pyridine (Intermediate 52) instead of 3-bromo-2-(5-fluoropyridin-2-yl)-6,7-dihydro-4H-pyrazolo[5,1-c][1,4]oxazine (Intermediate 37) and Pd(dppf)Cl₂ instead of CataCXium® A Pd G3 and also microwave irradiation at 90° C. for 2 h rather than conventional heating in Step A. MS (ESI): mass calcd. for C₂₀H₁₆F₃N₅ 383.1 m/z found 384.0 [M+H]⁺. ¹H NMR (400 MHz, DMSO-d₆) δ 13.37 (br s, 1H), 7.35-7.27 (m, 2H), 7.23 (s, 1H), 7.13-7.03 (m, 3H), 4.73 (t, J=12.6 Hz, 2H), 2.98 (t, J=6.6 Hz, 2H), 2.58 (s, 3H), 2.48-2.37 (m, 2H).

### Example 44: 4-(6,6-Difluoro-2-(5-fluoropyridin-2-yl)-4,5,6,7-tetrahydropyrazolo[1,5-a]pyridin-3-yl)-1H-pyrazolo[3,4-b]pyridine

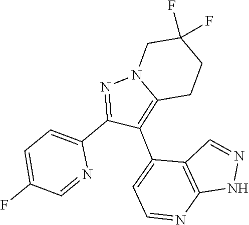 [SMILES: Fc1ccc(-c2nn3c(c2-c2ccnc4[nH]ncc24)CCC(F)(F)C3)nc1]

The title compound was prepared in a manner analogous to Example 27, except using 3-Bromo-6,6-difluoro-2-(5-fluoropyridin-2-yl)-4,5,6,7-tetrahydropyrazolo[1,5-a]pyridine (Intermediate 53) instead of (4aR,5aR)-3-Bromo-2-(5-fluoropyridin-2-yl)-4,4a,5,5a-tetrahydrocyclopropa[4,5]pyrrolo[1,2-b]pyrazole (Intermediate 47) and 4-(4,4,5,5-tetramethyl-1,3,2-dioxaborolan-2-yl)-1-((2-(trimethylsilyl)ethoxy)methyl)-1H-pyrazolo[3,4-b]pyridine (Intermediate 23) instead of 6-methyl-4-(4,4,5,5-tetramethyl-1,3,2-dioxaborolan-2-yl)-1-((2-(trimethylsilyl)ethoxy)methyl)-1H-pyrazolo[3,4-b]pyridine (Intermediate 26). MS (ESI): mass calcd. for C₁₈H₁₃F₃N₆, 370.1; m/z found, 371.1 [M+H]⁺. ¹H NMR (400 MHz, CDCl₃): δ 10.76 (s, 1H), 8.50 (d, J=4.8 Hz, 1H), 8.20 (d, J=2.9 Hz, 1H), 7.47-7.36 (m, 2H), 7.25-7.19 (m, 1H), 6.98 (d, J=4.8 Hz, 1H), 4.57 (t, J=12.3 Hz, 2H), 2.93 (t, J=6.7 Hz, 2H), 2.39-2.20 (m, 2H).

### Example 45: 4-(6,6-Difluoro-2-(5-fluoropyridin-2-yl)-4,5,6,7-tetrahydropyrazolo[1,5-a]pyridin-3-yl)-6-methyl-1H-pyrazolo[3,4-b]pyridine

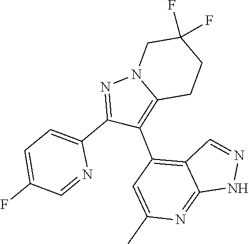 [SMILES: Cc1cc(-c2c(-c3ccc(F)cn3)nn3c2CCC(F)(F)C3)c2cn[nH]c2n1]

The title compound was prepared in a manner analogous to Example 27, except using 3-Bromo-6,6-difluoro-2-(5-fluoropyridin-2-yl)-4,5,6,7-tetrahydropyrazolo[1,5-a]pyridine (Intermediate 53) instead of (4aR,5aR)-3-Bromo-2-(5-fluoropyridin-2-yl)-4,4a,5,5a-tetrahydrocyclopropa[4,5]pyrrolo[1,2-b]pyrazole (Intermediate 47). MS (ESI): mass calcd. for C₁₉H₁₅F₃N₆, 384.1; m/z found, 385.2 [M+H]⁺. ¹H NMR (400 MHz, CDCl₃): δ 10.92 (s, 1H), 8.21 (d, J=2.9 Hz, 1H), 7.43-7.34 (m, 1H), 7.31 (s, 1H), 7.24-7.19 (m, 1H), 6.86 (s, 1H), 4.56 (t, J=12.3 Hz, 2H), 2.94 (t, J=6.7 Hz, 2H), 2.64 (s, 3H), 2.40-2.21 (m, 2H).

### Example 46: 4-(5,5-Difluoro-2-(4-fluorophenyl)-4,5,6,7-tetrahydropyrazolo[1,5-a]pyridin-3-yl)-1H-pyrazolo[3,4-b]pyridine

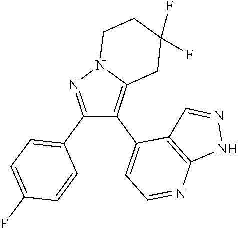 [SMILES: Fc1ccc(-c2nn3c(c2-c2ccnc4[nH]ncc24)CC(F)(F)CC3)cc1]

The title compound was prepared in a manner analogous to Example 1, Step A, except using 3-bromo-5,5-difluoro-2-(4-fluorophenyl)-4,5,6,7-tetrahydropyrazolo[1,5-a]pyridine (Intermediate 54) instead of 3-bromo-2-(5-fluoropyridin-2-yl)-6,7-dihydro-4H-pyrazolo[5,1-c][1,4]oxazine (Intermediate 37), 4-(4,4,5,5-tetramethyl-1,3,2-dioxaborolan-2-yl)-1-((2-(trimethylsilyl)ethoxy)methyl)-1H-pyrazolo[3,4-b]pyridine (Intermediate 23) instead of 4-(4,4,5,5-tetramethyl-1,3,2-dioxaborolan-2-yl)-1-((2-(trimethylsilyl)ethoxy)methyl)-1H-pyrrolo[2,3-b]pyridine (Intermediate 21), Pd(dppf)Cl₂ instead of CataCXium® A Pd G3 and also microwave irradiation at 90° C. for 2 h rather than conventional heating in Step A. LC-MS (ESI): mass calcd. for C₁₉H₁₄F₃N₅ 369.12 m/z found 370.0 [M+H]⁺. ¹H NMR (400 MHz, DMSO-d₆) δ 13.64 (s, 1H), 8.51 (d, J=4.8 Hz, 1H), 7.44 (s, 1H), 7.37-7.22 (m, 2H), 7.18-6.97 (m, 3H), 4.44 (t, J=6.4 Hz, 2H), 3.47 (t, J=14.1 Hz, 2H), 2.70 (tt, J=6.4, 12.9 Hz, 2H).

### Example 47: 4-(5,5-Difluoro-2-(4-fluorophenyl)-4,5,6,7-tetrahydropyrazolo[1,5-a]pyridin-3-yl)-6-methyl-1H-pyrazolo[3,4-b]pyridine

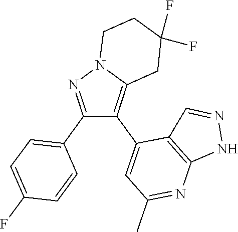 [SMILES: Cc1cc(-c2c(-c3ccc(F)cc3)nn3c2CC(F)(F)CC3)c2cn[nH]c2n1]

The title compound was prepared in a manner analogous to Example 1, Steps A-B, except using 3-bromo-5, 5-difluoro-2-(4-fluorophenyl)-4,5,6,7-tetrahydropyrazolo[1,5-a]pyridine (Intermediate 54) instead of 3-bromo-2-(5-fluoropyridin-2-yl)-6,7-dihydro-4H-pyrazolo[5,1-c][1,4]oxazine (Intermediate 37) and 6-methyl-4-(4,4,5,5-tetramethyl-1,3,2-dioxaborolan-2-yl)-1-((2-(trimethylsilyl)ethoxy)methyl)-1H-pyrazolo[3,4-b]pyridine (Intermediate 26) instead of 4-(4,4,5,5-tetramethyl-1,3,2-dioxaborolan-2-yl)-1-((2-(trimethylsilyl)ethoxy)methyl)-1H-pyrrolo[2,3-b]pyridine (Intermediate 21), Pd(dppf)Cl₂ instead of CataCXium® A Pd G3 and also microwave irradiation at 90° C. for 2 h rather than conventional heating in Step A. LC-MS (ESI): mass calcd. for C₂₀H₁₆F₃N₅ 383.14 m/z found 384.1 [M+H]⁺. ¹H NMR (400 MHz, DMSO-d₆) δ 13.39 (br s, 1H), 7.36-7.24 (m, 3H), 7.14-7.04 (m, 2H), 7.01 (s, 1H), 4.43 (t, J=6.4 Hz, 2H), 3.48 (t, J=14.1 Hz, 2H), 2.78-2.63 (m, 2H), 2.58 (s, 3H).

### Example 48: 4-[5,5-Difluoro-2-(5-fluoro-2-pyridyl)-6,7-dihydro-4H-pyrazolo[1,5-a]pyridin-3-yl]-1H-pyrazolo[3,4-b]pyridine

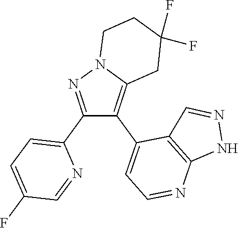 [SMILES: Fc1ccc(-c2nn3c(c2-c2ccnc4[nH]ncc24)CC(F)(F)CC3)nc1]

The title compound was prepared in a manner analogous to Example 1, Steps A-B, except using 4-(4,4,5,5-tetramethyl-1,3,2-dioxaborolan-2-yl)-1-((2-(trimethylsilyl)ethoxy)methyl)-1H-pyrazolo[3,4-b]pyridine (Intermediate 23) instead of 4-(4,4,5,5-tetramethyl-1,3,2-dioxaborolan-2-yl)-1-((2-(trimethylsilyl)ethoxy)methyl)-1H-pyrrolo[2,3-b]pyridine (Intermediate 21) and 3-bromo-5,5-difluoro-2-(5-fluoropyridin-2-yl)-4,5,6,7-tetrahydropyrazolo[1,5-a]pyridine. (Intermediate 55) instead of 3-bromo-2-(5-fluoropyridin-2-yl)-6,7-dihydro-4H-pyrazolo[5,1-c][1,4]oxazine (Intermediate 37) and Pd(dppf)Cl₂ instead of CataCXium® A Pd G3 and also microwave irradiation at 90° C. for 2 h rather than conventional heating in Step A. MS (ESI): mass calcd. for C₁₈H₁₃F₃N₆ 370.1 m/z found 371.0 [M+H]⁺. ¹H NMR (400 MHz, DMSO-d₆) δ 13.54 (br s, 1H), 8.47 (d, J=4.6 Hz, 1H), 8.22 (d, J=2.9 Hz, 1H), 7.89-7.82 (m, 1H), 7.80-7.70 (m, 1H), 7.37 (s, 1H), 7.07 (d, J=4.8 Hz, 1H), 4.46 (t, J=6.4 Hz, 2H), 3.47 (t, J=14.1 Hz, 2H), 2.79-2.62 (m, 2H).

### Example 49: 4-[2-(5-Fluoro-2-pyridyl)-6,6-dimethyl-5,7-dihydro-4H-pyrazolo[1,5-a]pyridin-3-yl]-1H-pyrazolo[3,4-b]pyridine

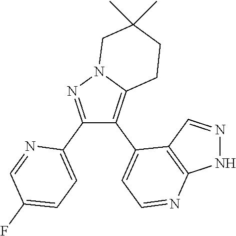 [SMILES: CC1(C)CCc2c(-c3ccnc4[nH]ncc34)c(-c3ccc(F)cn3)nn2C1]

The title compound was prepared in a manner analogous to Example 1, Step A-B except using 3-bromo-2-(5-fluoropyridin-2-yl)-6,6-dimethyl-4,5,6,7-tetrahydropyrazolo[1,5-a]pyridine (Intermediate 57) instead of 3-bromo-2-(5-fluoropyridin-2-yl)-6,7-dihydro-4H-pyrazolo[5,1-c][1,4]oxazine (Intermediate 37) and 4-(4,4,5,5-tetramethyl-1,3,2-dioxaborolan-2-yl)-1-((2-(trimethylsilyl)ethoxy)methyl)-1H-pyrazolo[3,4-b]pyridine (Intermediate 23) instead of -(4,4,5,5-tetramethyl-1,3,2-dioxaborolan-2-yl)-1-((2-(trimethylsilyl)ethoxy)methyl)-1H-pyrrolo[2,3-b]pyridine (Intermediate 21) in Step A and microwave irradiation at 90° C. for 1 h rather than conventional heat. MS (ESI): mass calcd. for C₂₀H₁₉FN₆ 362.2 m/z found 363.1 [M+H]⁺. ¹H NMR (400 MHz, DMSO-d₆) δ 13.48 (s, 1H), 8.45 (d, J=4.6 Hz, 1H), 8.21 (d, J=2.7 Hz, 1H), 7.91-7.79 (m, 1H), 7.75-7.71 (m, 1H), 7.40-7.25 (m, 1H), 7.09 (d, J=4.6 Hz, 1H), 3.95 (s, 2H), 2.78 (t, J=6.1 Hz, 2H), 1.65 (t, J=6.2 Hz, 2H), 1.10 (s, 6H).

### Example 50: 4-(2-(5-Fluoropyridin-2-yl)-5,5-dimethyl-4,5,6,7-tetrahydropyrazolo[1,5-a]pyridin-3-yl)-1H-pyrazolo[3,4-b]pyridine

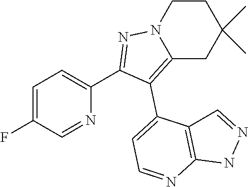 [SMILES: CC1(C)CCn2nc(-c3ccc(F)cn3)c(-c3ccnc4[nH]ncc34)c2C1]

The title compound was prepared in a manner analogous to Example 1, Steps A-B, except using 3-bromo-2-(5-fluoropyridin-2-yl)-5,5-dimethyl-4,5,6,7-tetrahydropyrazolo[1,5-a]pyridine (Intermediate 56) instead of 3-bromo-2-(5-fluoropyridin-2-yl)-6,7-dihydro-4H-pyrazolo[5,1-c][1,4]oxazine (Intermediate 37) and 4-(4,4,5,5-tetramethyl-1,3,2-dioxaborolan-2-yl)-1-((2-(trimethylsilyl)ethoxy)methyl)-1H-pyrazolo[3,4-b]pyridine (Intermediate 23) instead of 4-(4,4,5,5-tetramethyl-1,3,2-dioxaborolan-2-yl)-1-((2-(trimethylsilyl)ethoxy)methyl)-1H-pyrrolo[2,3-b]pyridine (Intermediate 21) and also microwave irradiation at 100° C. for 1 h rather than conventional heating in Step A. LC-MS (ESI): mass calcd. for C₂₀H₁₉FN₆ 362.17 m/z, found 363.1 [M+H]⁺. ¹H NMR (400 MHz, DMSO-d₆) δ 11.99 (br s, 1H), 8.59 (d, J=4.9 Hz, 1H), 8.33 (d, J=2.7 Hz, 1H), 7.53 (s, 1H), 7.42 (dd, J=4.4, 8.8 Hz, 1H), 7.28-7.23 (m, 1H), 7.04 (d, J=4.8 Hz, 1H), 4.37 (t, J=6.4 Hz, 2H), 2.60 (s, 2H), 1.96 (t, J=6.4 Hz, 2H), 1.10 (s, 6H).

### Example 51: (*)-4-[6-(Fluoromethyl)-2-(5-fluoro-2-pyridyl)-6-methyl-5,7-dihydro-4H-pyrazolo[1,5-a]pyridin-3-yl]-1H-pyrazolo[3,4-b]pyridine

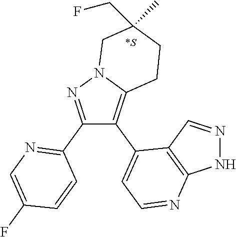 [SMILES: C[C@]1(CF)CCc2c(-c3ccnc4[nH]ncc34)c(-c3ccc(F)cn3)nn2C1]

The title compound was prepared in a manner analogous to Example 1, Steps A-B, except using 3-bromo-6-(fluoromethyl)-2-(5-fluoropyridin-2-yl)-6-methyl-4,5,6,7-tetrahydropyrazolo[1,5-a]pyridine (Intermediate 60) instead of 3-bromo-2-(5-fluoropyridin-2-yl)-6,7-dihydro-4H-pyrazolo[5,1-c][1,4]oxazine (Intermediate 37), 4-(4,4,5,5-tetramethyl-1,3,2-dioxaborolan-2-yl)-1-((2-(trimethylsilyl)ethoxy)methyl)-1H-pyrazolo[3,4-b]pyridine (Intermediate 23) instead of 4-(4,4,5,5-tetramethyl-1,3,2-dioxaborolan-2-yl)-1-((2-(trimethylsilyl)ethoxy)methyl)-1H-pyrrolo[2,3-b]pyridine (Intermediate 21), and also microwave irradiation at 90° C. for 1 h rather than conventional heating in Step A. The final compound was obtained by SFC purification Method G. The pure fractions were collected, and the volatiles were removed under reduced pressure. The product was suspended in water (10 mL), the mixture frozen using dry ice/acetone, and then lyophilized to dryness to afford the title compound (40.0 mg, >99% purity, 27% yield) as a white solid. MS (ESI): mass calcd. for C₂₀H₁₈F₂N₆ 380.2 m/z found 381.0 [M+H]⁺. ¹H NMR (400 MHz, DMSO-d₆) δ 13.48 (s, 1H), 8.45 (d, J=4.6 Hz, 1H), 8.22 (d, J=2.7 Hz, 1H), 7.92-7.65 (m, 2H), 7.35 (s, 1H), 7.09 (d, J=4.9 Hz, 1H), 4.62-4.33 (m, 2H), 4.22-3.98 (m, 2H), 2.81 (t, J=6.4 Hz, 2H), 1.91-1.63 (m, 2H), 1.14 (s, 3H).

### Example 52: (*R)-4-[6-(Fluoromethyl)-2-(5-fluoro-2-pyridyl)-6-methyl-5,7-dihydro-4H-pyrazolo[1,5-a]pyridin-3-yl]-1H-pyrazolo[3,4-b]pyridine

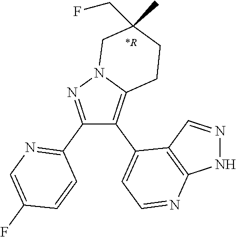 [SMILES: C[C@@]1(CF)CCc2c(-c3ccnc4[nH]ncc34)c(-c3ccc(F)cn3)nn2C1]

The title compound was prepared in a manner analogous to Example 1, Steps A-B, except using 3-bromo-6-(fluoromethyl)-2-(5-fluoropyridin-2-yl)-6-methyl-4,5,6,7-tetrahydropyrazolo[1,5-a]pyridine (Intermediate 60) instead of 3-bromo-2-(5-fluoropyridin-2-yl)-6,7-dihydro-4H-pyrazolo[5,1-c][1,4]oxazine (Intermediate 37), 4-(4,4,5,5-tetramethyl-1,3,2-dioxaborolan-2-yl)-1-((2-(trimethylsilyl)ethoxy)methyl)-1H-pyrazolo[3,4-b]pyridine (Intermediate 23) instead of 4-(4,4,5,5-tetramethyl-1,3,2-dioxaborolan-2-yl)-1-((2-(trimethylsilyl)ethoxy)methyl)-1H-pyrrolo[2,3-b]pyridine (Intermediate 21), and also microwave irradiation at 90° C. for 1 h rather than conventional heating in Step A. The final compound was obtained by SFC purification Method G. The pure fractions were collected, and the volatiles were removed under reduced pressure. The product was suspended in water (10 mL), the mixture frozen using dry ice/acetone, and then lyophilized to dryness to afford the title compound as a white solid (42.3 mg, >99% purity, 28% yield). MS (ESI): mass calcd. for C₂₀H₁₈F₂N₆ 380.2 m/z found 381.0 [M+H]⁺. ¹H NMR (400 MHz, DMSO-d₆) δ 13.46 (s, 1H), 8.45 (d, J=4.6 Hz, 1H), 8.22 (d, J=2.6 Hz, 1H), 7.88-7.69 (m, 2H), 7.35 (s, 1H), 7.09 (d, J=4.6 Hz, 1H), 4.58-4.31 (m, 2H), 4.24-3.93 (m, 2H), 2.81 (t, J=6.4 Hz, 2H), 1.94-1.65 (m, 2H), 1.14 (s, 3H).

### Example 53: (*S)-4-(6-Fluoro-2-(5-fluoropyridin-2-yl)-6-(methoxymethyl)-4,5,6,7-tetrahydropyrazolo[1,5-a]pyridin-3-yl)-6-methyl-1H-pyrazolo[3,4-b]pyridine

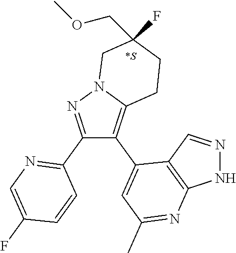 [SMILES: COC[C@]1(F)CCc2c(-c3cc(C)nc4[nH]ncc34)c(-c3ccc(F)cn3)nn2C1]

The title compound was prepared in a manner analogous to Example 1, Steps A-B, except using 3-bromo-6-fluoro-2-(5-fluoropyridin-2-yl)-6-(methoxymethyl)-4,5,6,7-tetrahydropyrazolo[1,5-a]pyridine (Intermediate 61) instead of 3-bromo-2-(5-fluoropyridin-2-yl)-6,7-dihydro-4H-pyrazolo[5,1-c][1,4]oxazine (Intermediate 37), 6-methyl-4-(4,4,5,5-tetramethyl-1,3,2-dioxaborolan-2-yl)-1-((2-(trimethylsilyl)ethoxy)methyl)-1H-pyrazolo[3,4-b]pyridine (Intermediate 26) instead of 4-(4,4,5,5-tetramethyl-1,3,2-dioxaborolan-2-yl)-1-((2-(trimethylsilyl)ethoxy)methyl)-1H-pyrrolo[2,3-b]pyridine (Intermediate 21) and also microwave irradiation at 90° C. for 2 h rather than conventional heating in Step A. The product of Step B was purified by preparative SFC over DAICEL CHIRALPAK AD (250 mm*30 mm, 10 um) (isocratic elution: EtOH (containing 0.1% aq. NH₃): supercritical CO₂, 45%: 55% to 45%: 55% (v/v)). The pure fractions were collected and the volatiles were removed under reduced pressure. The product was suspended in water (10 mL), the mixture frozen using dry ice/EtOH, and then lyophilized to dryness to afford the title compound (11.7 mg, 10%) as a white solid. MS (ESI): mass calcd. for C₂₁H₂₀F₂N₆O 410.2 m/z found 411.1 [M+H]⁺. ¹H NMR (400 MHz, DMSO-d₆) δ 13.26 (s, 1H), 8.23 (d, J=2.5 Hz, 1H), 7.85-7.71 (m, 2H), 7.16-6.99 (m, 2H), 4.51-4.32 (m, 2H), 3.76-3.64 (m, 2H), 3.40 (s, 3H), 2.98-2.80 (m, 2H), 2.58 (s, 3H), 2.27-1.84 (m, 2H).

### Example 54: (*R)-4-(6-Fluoro-2-(5-fluoropyridin-2-yl)-6-(methoxymethyl)-4,5,6,7-tetrahydropyrazolo[1,5-a]pyridin-3-yl)-6-methyl-1H-pyrazolo[3,4-b]pyridine

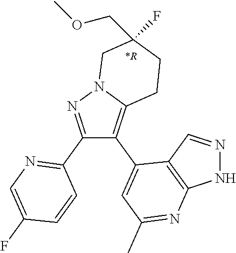 [SMILES: COC[C@@]1(F)CCc2c(-c3cc(C)nc4[nH]ncc34)c(-c3ccc(F)cn3)nn2C1]

The title compound was prepared in a manner analogous Example 1, Steps A-B, except using 3-bromo-6-fluoro-2-(5-fluoropyridin-2-yl)-6-(methoxymethyl)-4,5,6,7-tetrahydropyrazolo[1,5-a]pyridine (Intermediate 61) instead of 3-bromo-2-(5-fluoropyridin-2-yl)-6,7-dihydro-4H-pyrazolo[5,1-c][1,4]oxazine (Intermediate 37), 6-methyl-4-(4,4,5,5-tetramethyl-1,3,2-dioxaborolan-2-yl)-1-((2-(trimethylsilyl)ethoxy)methyl)-1H-pyrazolo[3,4-b]pyridine (Intermediate 26) instead of 4-(4,4,5,5-tetramethyl-1,3,2-dioxaborolan-2-yl)-1-((2-(trimethylsilyl)ethoxy)methyl)-1H-pyrrolo[2,3-b]pyridine (Intermediate 21) and also microwave irradiation at 90° C. for 2 h rather than conventional heating in Step A. The product of Step B was purified by preparative SFC over DAICEL CHIRALPAK AD (250 mm*30 mm, 10 um) (isocratic elution: EtOH (containing 0.1% aq. NH₃): supercritical CO₂, 45%: 55% to 45%: 55% (v/v)). The pure fractions were collected and the volatiles were removed under reduced pressure. The product was suspended in water (10 mL), the mixture frozen using dry ice/EtOH, and then lyophilized to dryness to afford the title compound (24.6 mg, 20%) as a white solid. MS (ESI): mass calcd. for C₂₁H₂₀F₂N₆O 410.17 m/z found 411.1 [M+H]⁺. ¹H NMR (400 MHz, DMSO-d₆) δ 13.26 (s, 1H), 8.23 (d, J=2.7 Hz, 1H), 7.85-7.70 (m, 2H), 7.15-7.00 (m, 2H), 4.51-4.34 (m, 2H), 3.77-3.62 (m, 2H), 3.39 (s, 3H), 3.01-2.80 (m, 2H), 2.58 (s, 3H), 2.24-1.88 (m, 2H).

### Example 55: (*S)-4-(2-(5-Fluoropyridin-2-yl)-6-(methoxymethyl)-6-methyl-4,5,6,7-tetrahydropyrazolo[1,5-a]pyridin-3-yl)-6-methyl-1H-pyrazolo[3,4-b]pyridine

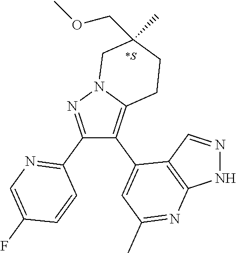 [SMILES: COC[C@@]1(C)CCc2c(-c3cc(C)nc4[nH]ncc34)c(-c3ccc(F)cn3)nn2C1]

The title compound was prepared in a manner analogous to Example 1, Steps A-B, except using 3-bromo-2-(5-fluoropyridin-2-yl)-6-(methoxymethyl)-6-methyl-4,5,6,7-tetrahydropyrazolo[1,5-a]pyridine (Intermediate 62) instead of 3-bromo-2-(5-fluoropyridin-2-yl)-6,7-dihydro-4H-pyrazolo[5,1-c][1,4]oxazine (Intermediate 37), 6-methyl-4-(4,4,5,5-tetramethyl-1,3,2-dioxaborolan-2-yl)-1-((2-(trimethylsilyl)ethoxy)methyl)-1H-pyrazolo[3,4-b]pyridine (Intermediate 26) instead of 4-(4,4,5,5-tetramethyl-1,3,2-dioxaborolan-2-yl)-1-((2-(trimethylsilyl)ethoxy)methyl)-1H-pyrrolo[2,3-b]pyridine (Intermediate 21) and also microwave irradiation at 90° C. for 1 h rather than conventional heating in Step A. The product of Step B was purified by preparative SFC over DAICEL CHIRALPAK AD-H (250 mm*30 mm, 5 um) (isocratic elution: EtOH (containing 0.1% aq. NH₃): supercritical CO₂, 45%: 55% to 45%: 55% (v/v)). The pure fractions were collected and the volatiles were removed under reduced pressure. The product was suspended in water (10 mL), the mixture frozen using dry ice/EtOH, and then lyophilized to dryness to afford the title compound (72.3 mg, 20%) as a white solid. MS (ESI): mass calcd. for C₂₂H₂₃FN₆O 406.2 m/z found 407.1 [M+H]⁺. ¹H NMR (400 MHz, DMSO-d₆) δ 13.24 (s, 1H), 8.22 (m, 1H), 7.85-7.69 (m, 2H), 7.16 (s, 1H), 7.01 (s, 1H), 4.14-3.88 (m, 2H), 3.35 (m, 5H), 2.86-2.73 (m, 2H), 2.57 (s, 3H), 1.84-1.57 (m, 2H), 1.10 (s, 3H).

### Example 56: (*R)-4-(2-(S-Fluoropyridin-2-yl)-6-(methoxymethyl)-6-methyl-4,5,6,7-tetrahydropyrazolo[1,5-a]pyridin-3-yl)-6-methyl-1H-pyrazolo[3,4-b]pyridine

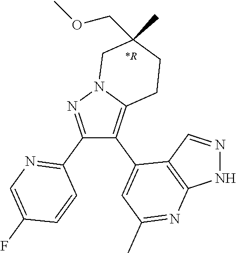 [SMILES: COC[C@]1(C)CCc2c(-c3cc(C)nc4[nH]ncc34)c(-c3ccc(F)cn3)nn2C1]

The title compound was prepared in a manner analogous Example 1, Steps A-B, except using 3-bromo-2-(5-fluoropyridin-2-yl)-6-(methoxymethyl)-6-methyl-4,5,6,7-tetrahydropyrazolo[1,5-a]pyridine (Intermediate 62) instead of 3-bromo-2-(5-fluoropyridin-2-yl)-6,7-dihydro-4H-pyrazolo[5,1-c][1,4]oxazine (Intermediate 37), 6-methyl-4-(4,4,5,5-tetramethyl-1,3,2-dioxaborolan-2-yl)-1-((2-(trimethylsilyl)ethoxy)methyl)-1H-pyrazolo[3,4-b]pyridine (Intermediate 26) instead of 4-(4,4,5,5-tetramethyl-1,3,2-dioxaborolan-2-yl)-1-((2-(trimethylsilyl)ethoxy)methyl)-1H-pyrrolo[2,3-b]pyridine (Intermediate 21) and also microwave irradiation at 90° C. for 1 h rather than conventional heating in Step A. The product of Step B was purified by preparative SFC over DAICEL CHIRALPAK AD-H (250 mm*30 mm, 5 um) (isocratic elution: EtOH (containing 0.1% aq. NH₃): supercritical CO₂, 45%: 55% to 45%: 55% (v/v)). The pure fractions were collected and the volatiles were removed under reduced pressure. The product was suspended in water (10 mL), the mixture frozen using dry ice/EtOH, and then lyophilized to dryness to afford the title compound (93.9 mg, 26%) as a white solid. MS (ESI): mass calcd. for C₂₂H₂₃FN₆O 406.2 m/z found 407.1 [M+H]⁺. ¹H NMR (400 MHz, DMSO-d₆) δ 13.24 (s, 1H), 8.22 (m, 1H), 7.85-7.69 (m, 2H), 7.16 (s, 1H), 7.01 (s, 1H), 4.14-3.88 (m, 2H), 3.35 (m, 5H), 2.86-2.73 (m, 2H), 2.57 (s, 3H), 1.84-1.57 (m, 2H), 1.10 (s, 3H).

### Example 57: (Racemic) 2-(5-Fluoropyridin-2-yl)-3-(6-methyl-1H-pyrazolo[3,4-b]pyridin-4-yl)-5,5a,6,6a-tetrahydro-4H-cyclopropa[e]pyrazolo[1,5-a]pyridine

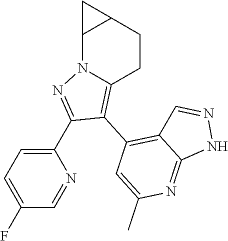 [SMILES: Cc1cc(-c2c(-c3ccc(F)cn3)nn3c2CCC2CC23)c2cn[nH]c2n1]

The title compound was prepared in a manner analogous to Example 27, Steps A-B, except using racemic (5aR,6aS)-3-bromo-2-(5-fluoropyridin-2-yl)-5,5a,6,6a-tetrahydro-4H-cyclopropa[e]pyrazolo[1,5-a]pyridine (Intermediate 63) instead of (4aR,5aR)-3-bromo-2-(5-fluoropyridin-2-yl)-4,4a,5,5a-tetrahydrocyclopropa[4,5]pyrrolo[1,2-b]pyrazole (Intermediate 47) in Step A. MS (ESI): mass calcd. for C₂₀H₁₇FN₆, 360.2; m/z found, 361.2 [M+H]⁺. ¹H NMR (400 MHz, CDCl₃): δ 11.09 (s, 1H), 8.19 (d, J=2.8 Hz, 1H), 7.44 (dd, J=8.8, 4.4 Hz, 1H), 7.30 (s, 1H), 7.24-7.18 (m, 1H), 6.80 (s, 1H), 3.96-3.84 (m, 1H), 2.82-2.68 (m, 1H), 2.62 (s, 3H), 2.52-2.37 (m, 1H), 2.11-1.91 (m, 2H), 1.70 (d, J=13.3 Hz, 1H), 1.19-1.04 (m, 1H), 1.00-0.86 (m, 1H).

### Example 58: (Racemic) 6,6-Difluoro-2-(5-fluoropyridin-2-yl)-3-(1H-pyrazolo[3,4-b]pyridin-4-yl)-5,5a,6,6a-tetrahydro-4H-cyclopropa[e]pyrazolo[1,5-a]pyridine

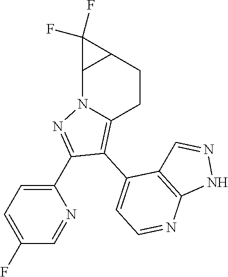 [SMILES: Fc1ccc(-c2nn3c(c2-c2ccnc4[nH]ncc24)CCC2C3C2(F)F)nc1]

The title compound was prepared in a manner analogous to Example 27, Steps A-B, except using racemic (5aR,6aS)-3-bromo-6,6-difluoro-2-(5-fluoropyridin-2-yl)-5,5a,6,6a-tetrahydro-4H-cyclopropa[e]pyrazolo[1,5-a]pyridine (Intermediate 64) instead of (4aR,5aR)-3-bromo-2-(5-fluoropyridin-2-yl)-4,4a,5,5a-tetrahydrocyclopropa[4,5]pyrrolo[1,2-b]pyrazole (Intermediate 47) and 4-(4,4,5,5-tetramethyl-1,3,2-dioxaborolan-2-yl)-1-((2-(trimethylsilyl)ethoxy)methyl)-1H-pyrazolo[3,4-b]pyridine (Intermediate 23) instead of 6-methyl-4-(4,4,5,5-tetramethyl-1,3,2-dioxaborolan-2-yl)-1-((2-(trimethylsilyl)ethoxy)methyl)-1H-pyrazolo[3,4-b]pyridine (Intermediate 26) in Step A. MS (ESI): mass calcd. for C₁₉H₁₃F₃N₆, 382.1; m/z found, 383.1 [M+H]⁺. ¹H NMR (400 MHz, CDCl₃): δ 10.72 (s, 1H), 8.47 (d, J=4.8 Hz, 1H), 8.17 (d, J=2.9 Hz, 1H), 7.54-7.46 (m, 1H), 7.43 (s, 1H), 7.29-7.20 (m, 1H), 6.94 (d, J=4.8 Hz, 1H), 4.30 (dd, J=10.8, 6.4 Hz, 1H), 2.82-2.59 (m, 2H), 2.46-2.34 (m, 1H), 2.08 (t, J=6.1 Hz, 2H).

### Example 59: (Racemic) 6,6-Difluoro-2-(5-fluoropyridin-2-yl)-3-(6-methyl-1H-pyrazolo[3,4-b]pyridin-4-yl)-5,5a,6,6a-tetrahydro-4H-cyclopropa[e]pyrazolo[1,5-a]pyridine

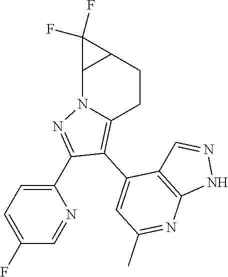 [SMILES: Cc1cc(-c2c(-c3ccc(F)cn3)nn3c2CCC2C3C2(F)F)c2cn[nH]c2n1]

The title compound was prepared in a manner analogous to Example 27, Steps A-B, except using racemic (5aR,6aS)-3-bromo-6,6-difluoro-2-(5-fluoropyridin-2-yl)-5,5a,6,6a-tetrahydro-4H-cyclopropa[e]pyrazolo[1,5-a]pyridine (Intermediate 64) instead of (4aR,5aR)-3-Bromo-2-(5-fluoropyridin-2-yl)-4,4a,5,5a-tetrahydrocyclopropa[4,5]pyrrolo[1,2-b]pyrazole (Intermediate 47) in Step A. MS (ESI): mass calcd. for C₂₀H₁₅F₃N₆, 396.1; m/z found, 397.1 [M+H]⁺. ¹H NMR (400 MHz, CDCl₃): δ 10.66 (s, 1H), 8.19 (d, J=2.9 Hz, 1H), 7.49 (dd, J=8.7, 4.4 Hz, 1H), 7.32 (s, 1H), 7.26-7.20 (m, 1H), 6.82 (s, 1H), 4.30 (dd, J=10.8, 6.4 Hz, 1H), 2.85-2.63 (m, 2H), 2.61 (s, 3H), 2.49-2.31 (m, 1H), 2.20-1.93 (m, 2H).

### Example 60: 4*R,7*S)-2-(4-Fluorophenyl)-3-(1H-pyrazolo[3,4-b]pyridin-4-yl)-4,5,6,7-tetrahydro-4,7-methanopyrazolo[1,5-a]pyridine

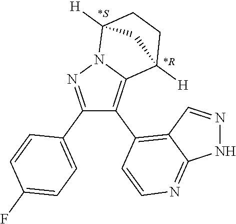 [SMILES: Fc1ccc(-c2nn3c(c2-c2ccnc4[nH]ncc24)[C@@H]2CC[C@H]3C2)cc1]

The title compound was prepared in a manner analogous to Example 1, Steps A-B, except using 3-bromo-2-(4-fluorophenyl)-4,5,6,7-tetrahydro-4,7-methanopyrazolo[1,5-a]pyridine (Intermediate 143) instead of 3-bromo-2-(5-fluoropyridin-2-yl)-6,7-dihydro-4H-pyrazolo[5,1-c][1,4]oxazine (Intermediate 37), 4-(4,4,5,5-tetramethyl-1,3,2-dioxaborolan-2-yl)-1-((2-(trimethylsilyl)ethoxy)methyl)-1H-pyrazolo[3,4-b]pyridine (Intermediate 23) instead of 4-(4,4,5,5-tetramethyl-1,3,2-dioxaborolan-2-yl)-1-((2-(trimethylsilyl)ethoxy)methyl)-1H-pyrrolo[2,3-b]pyridine (Intermediate 21), and also microwave irradiation at 100° C. for 1 h instead of conventional heating in Step A. The racemic product, 2-(4-Fluorophenyl)-3-(1H-pyrazolo[3,4-b]pyridin-4-yl)-4,5,6,7-tetrahydro-4,7-methanopyrazolo[1,5-a]pyridine (170 mg, 0.49 mmol) was purified by SFC Method E. The pure fractions were collected and the volatiles were removed under reduced pressure. The product was suspended in water (20 mL), the mixture frozen using dry ice/acetone, and then lyophilized to dryness to afford the title compound as a white solid (43 mg, 25%) and its enantiomer (Example 61). MS (ESI): mass calcd. for C₂₀H₁₆FN₅ 345.1; m/z found 346.2 [M+H]⁺. ¹H NMR (400 MHz, DMSO-d₆) δ 13.56 (s, 1H), 8.39 (d, J=4.6 Hz, 1H), 7.50 (d, J=1.1 Hz, 1H), 7.35-7.25 (m, 2H), 7.13-7.02 (m, 2H), 6.89 (d, J=4.9 Hz, 1H), 4.97 (s, 1H), 3.65 (s, 1H), 2.31-2.18 (m, 1H), 2.05-1.92 (m, 2H), 1.86 (d, J=9.0 Hz, 1H), 1.29-1.17 (m, 1H), 1.16-1.07 (m, 1H).

### Example 61: (4*S,7*R)-2-(4-Fluorophenyl)-3-(1H-pyridin-4-yl)-4,5,6,7-tetrahydro-4,7-methanopyrazolo[1,5-a]pyridine

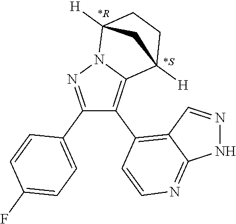 [SMILES: Fc1ccc(-c2nn3c(c2-c2ccnc4[nH]ncc24)[C@H]2CC[C@@H]3C2)cc1]

2-(4-Fluorophenyl)-3-(1H-pyrazolo[3,4-b]pyridin-4-yl)-4,5,6,7-tetrahydro-4,7-methanopyrazolo[1,5-a]pyridine (see Example 60, 170 mg, 0.49 mmol) was purified by SFC Method E. The pure fractions were collected and the volatiles were removed under reduced pressure. The product was suspended in water (20 mL), the mixture frozen using dry ice/acetone, and then lyophilized to dryness to afford the title compound as a white solid (43 mg, 25%). MS (ESI): mass calcd. for C₂₀H₁₆FN₅ 345.1; m/z found 346.2 [M+H]⁺. ¹H NMR (400 MHz, DMSO-d₆) δ 13.60 (s, 1H), 8.42 (d, J=4.6 Hz, 1H), 7.54 (s, 1H), 7.40-7.28 (m, 2H), 7.16-7.03 (m, 2H), 6.93 (d, J=4.9 Hz, 1H), 5.01 (s, 1H), 3.69 (s, 1H), 2.34-2.19 (m, 1H), 2.07-1.96 (m, 2H), 1.90 (d, J=9.0 Hz, 1H), 1.33-1.21 (m, 1H), 1.21-1.09 (m, 1H).

### Example 62: (4*R,7*S)-2-(5-Fluoropyridin-2-yl)-3-(1H-pyrazolo[3,4-b]pyridin-4-yl)-4,5,6,7-tetrahydro-4,7-methanopyrazolo[1,5-a]pyridine

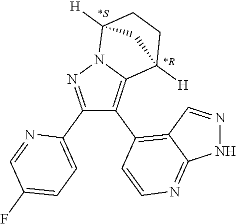 [SMILES: Fc1ccc(-c2nn3c(c2-c2ccnc4[nH]ncc24)[C@@H]2CC[C@H]3C2)nc1]

The title compound was prepared in a manner analogous to Example 1, Steps A-B, except using 3-bromo-2-(5-fluoropyridin-2-yl)-4,5,6,7-tetrahydro-4,7-methanopyrazolo[1,5-a]pyridine (Intermediate 146) instead of 3-bromo-2-(5-fluoropyridin-2-yl)-6,7-dihydro-4H-pyrazolo[5,1-c][1,4]oxazine (Intermediate 37), 4-(4,4,5,5-tetramethyl-1,3,2-dioxaborolan-2-yl)-1-((2-(trimethylsilyl)ethoxy)methyl)-1H-pyrazolo[3,4-b]pyridine (Intermediate 23) instead of 4-(4,4,5,5-tetramethyl-1,3,2-dioxaborolan-2-yl)-1-((2-(trimethylsilyl)ethoxy)methyl)-1H-pyrrolo[2,3-b]pyridine (Intermediate 21), and also microwave irradiation at 100° C. for 1 h instead of conventional heating in Step A. The racemic product, 2-(5-fluoropyridin-2-yl)-3-(1H-pyrazolo[3,4-b]pyridin-4-yl)-4,5,6,7-tetrahydro-4,7-methanopyrazolo[1,5-a]pyridine was purified by SFC Method D. The pure fractions were collected, and the volatiles were removed under reduced pressure. The product was suspended in water (20 mL), the mixture was frozen using dry ice/acetone, then lyophilized to dryness to afford the title compound as a white solid (22 mg, 18%) and its enantiomer. MS (ESI): mass calcd. for C₁₉H₁₅FN₆ 346.1; m/z found 347.1 [M+H]⁺. ¹H NMR (400 MHz, DMSO-d₆) δ 13.52 (s, 1H), 8.41 (d, J=4.6 Hz, 1H), 8.27 (d, J=2.9 Hz, 1H), 7.89-7.82 (m, 1H), 7.81-7.72 (m, 1H), 7.45 (s, 1H), 6.98 (d, J=4.9 Hz, 1H), 5.04 (s, 1H), 3.69 (s, 1H), 2.34-2.23 (m, 1H), 2.08-1.97 (m, 2H), 1.92 (d, J=9.5 Hz, 1H), 1.33-1.23 (m, 1H), 1.23-1.12 (m, 1H).

### Example 63: (4*S,7*R)-2-(5-Fluoropyridin-2-yl)-3-(1H-pyrazolo[3,4-b]pyridin-4-yl)-4,5,6,7-tetrahydro-4,7-methanopyrazolo[1,5-a]pyridine

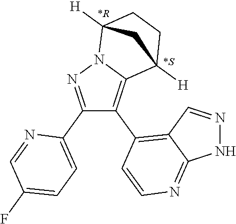 [SMILES: Fc1ccc(-c2nn3c(c2-c2ccnc4[nH]ncc24)[C@H]2CC[C@@H]3C2)nc1]

2-(5-Fluoropyridin-2-yl)-3-(1H-pyrazolo[3,4-b]pyridin-4-yl)-4,5,6,7-tetrahydro-4,7-methanopyrazolo[1,5-a]pyridine (see Example 62) was purified by purified by SFC Method D. The pure fractions were collected, and the volatiles were removed under reduced pressure. The product was suspended in water (20 mL), the mixture frozen using dry ice/acetone, then lyophilized to dryness to afford the title compound as a white solid. MS (ESI): mass calcd. for C₁₉H₁₅FN₆ 346.1; m/z found 347.1 [M+H]⁺. ¹H NMR (400 MHz, DMSO-d₆) δ 13.49 (s, 1H), 8.37 (d, J=4.6 Hz, 1H), 8.24 (d, J=2.9 Hz, 1H), 7.84-7.78 (m, 1H), 7.78-7.69 (m, 1H), 7.41 (d, J=1.2 Hz, 1H), 6.95 (d, J=4.6 Hz, 1H), 5.00 (s, 1H), 3.65 (s, 1H), 2.33-2.18 (m, 1H), 2.06-1.93 (m, 2H), 1.88 (d, J=9.0 Hz, 1H), 1.25-1.15 (m, 2H).

### Example 64: 2-(4-Fluorophenyl)-3-(1H-pyrazolo[3,4-b]pyridin-4-yl)-4,5,6,7-tetrahydro-4,7-ethanopyrazolo[1,5-a]pyridine

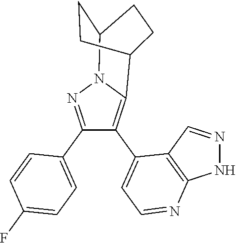 [SMILES: Fc1ccc(-c2nn3c(c2-c2ccnc4[nH]ncc24)C2CCC3CC2)cc1]

The title compound was prepared in a manner analogous to Example 1, Steps A-B, except using 3-bromo-2-(4-fluorophenyl)-4,5,6,7-tetrahydro-4,7-ethanopyrazolo[1,5-a]pyridine (Intermediate 145) instead of 3-bromo-2-(5-fluoropyridin-2-yl)-6,7-dihydro-4H-pyrazolo[5,1-c][1,4]oxazine (Intermediate 37), 4-(4,4,5,5-tetramethyl-1,3,2-dioxaborolan-2-yl)-1-((2-(trimethylsilyl)ethoxy)methyl)-1H-pyrazolo[3,4-b]pyridine (Intermediate 23) instead of 4-(4,4,5,5-tetramethyl-1,3,2-dioxaborolan-2-yl)-1-((2-(trimethylsilyl)ethoxy)methyl)-1H-pyrrolo[2,3-b]pyridine (Intermediate 21), and also microwave irradiation at 100° C. for 1 h instead of conventional heating in Step A. MS (ESI): mass calcd. for C₂₁H₁₈FN₅ 359.2; m/z found 360.1 [M+H]⁺. ¹H NMR (400 MHz, DMSO-d₆) δ 8.45 (d, J=4.6 Hz, 1H), 7.51 (s, 1H), 7.37-7.30 (m, 2H), 7.13-7.07 (m, 2H), 6.94 (d, J=4.6 Hz, 1H), 4.86-4.81 (m, 1H), 3.31-3.28 (m, 1H), 2.03-1.87 (m, 4H), 1.79-1.70 (m, 2H), 1.66-1.57 (m, 2H). N—H proton is not observed.

### Example 65: 2-(5-Fluoropyridin-2-yl)-3-(1H-pyrazolo[3,4-b]pyridin-4-yl)-4,5,6,7-tetrahydro-4,7-ethanopyrazolo[1,5-a]pyridine

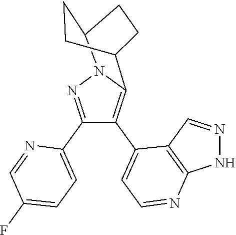 [SMILES: Fc1ccc(-c2nn3c(c2-c2ccnc4[nH]ncc24)C2CCC3CC2)nc1]

The title compound was prepared in a manner analogous to Example 1, Steps A-B, except using 3-bromo-2-(5-fluoropyridin-2-yl)-4,5,6,7-tetrahydro-4,7-ethanopyrazolo[1,5-a]pyridine (Intermediate 85) instead of 3-bromo-2-(5-fluoropyridin-2-yl)-6,7-dihydro-4H-pyrazolo[5,1-c][1,4]oxazine (Intermediate 37), 4-(4,4,5,5-tetramethyl-1,3,2-dioxaborolan-2-yl)-1-((2-(trimethylsilyl)ethoxy)methyl)-1H-pyrazolo[3,4-b]pyridine (Intermediate 23) instead of 4-(4,4,5,5-tetramethyl-1,3,2-dioxaborolan-2-yl)-1-((2-(trimethylsilyl)ethoxy)methyl)-1H-pyrrolo[2,3-b]pyridine (Intermediate 21), and also microwave irradiation at 100° C. for 1 h instead of conventional heating in Step A. MS (ESI): mass calcd. for C₂₀H₁₇FN₆ 360.2; m/z found 361.1 [M+H]⁺. ¹H NMR (400 MHz, DMSO-d₆) δ 8.42 (d, J=4.9 Hz, 1H), 8.25 (d, J=2.9 Hz, 1H), 7.88-7.82 (m, 1H), 7.79-7.72 (m, 1H), 7.43 (s, 1H), 6.97 (d, J=4.9 Hz, 1H), 4.89-4.80 (m, 1H), 3.31-3.28 (m, 1H), 2.04-1.87 (m, 4H), 1.81-1.70 (m, 2H), 1.67-1.56 (m, 2H). N—H proton is not observed.

### Example 66: 2-(4-Fluorophenyl)-3-(6-methyl-1H-pyrazolo[3,4-b]pyridin-4-yl)-4,5,6,7-tetrahydro-4,7-ethanopyrazolo[1,5-a]pyridine

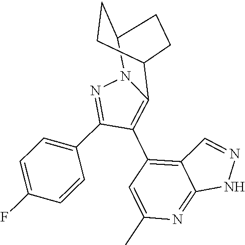 [SMILES: Cc1cc(-c2c(-c3ccc(F)cc3)nn3c2C2CCC3CC2)c2cn[nH]c2n1]

The title compound was prepared in a manner analogous to Example 1, Steps A-B, except using 3-bromo-2-(4-fluorophenyl)-4,5,6,7-tetrahydro-4,7-ethanopyrazolo[1,5-a]pyridine (Intermediate 145) instead of 3-bromo-2-(5-fluoropyridin-2-yl)-6,7-dihydro-4H-pyrazolo[5,1-c][1,4]oxazine (Intermediate 37), 6-methyl-4-(4,4,5,5-tetramethyl-1,3,2-dioxaborolan-2-yl)-1-((2-(trimethylsilyl)ethoxy)methyl)-1H-pyrazolo[3,4-b]pyridine (Intermediate 26) instead of 4-(4,4,5,5-tetramethyl-1,3,2-dioxaborolan-2-yl)-1-((2-(trimethylsilyl)ethoxy)methyl)-1H-pyrrolo[2,3-b]pyridine (Intermediate 21), and also microwave irradiation at 100° C. for 1 h instead of conventional heating in Step A. MS (ESI): mass calcd. for C₂₂H₂₀FN₅ 373.2; m/z found 374.1 [M+H]⁺. ¹H NMR (400 MHz, DMSO-d₆) δ 13.37 (br s, 1H), 7.38-7.29 (m, 3H), 7.13-7.05 (m, 2H), 6.85 (s, 1H), 4.85-4.78 (m, 1H), 3.31-3.28 (m, 1H), 2.54 (s, 3H), 2.03-1.86 (m, 4H), 1.79-1.68 (m, 2H), 1.66-1.54 (m, 2H).

### Example 67: 2-(4-Chlorophenyl)-3-(1H-pyrazolo[3,4-b]pyridin-4-yl)-6,7-dihydro-4H-pyrazolo[5,1-c][1,4]oxazine

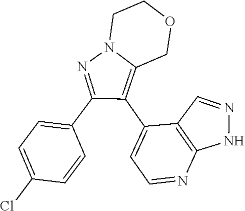 [SMILES: Clc1ccc(-c2nn3c(c2-c2ccnc4[nH]ncc24)COCC3)cc1]

The title compound was prepared in a manner analogous to Example 1, Steps A-B except using 3-bromo-2-(4-chlorophenyl)-6,7-dihydro-4H-pyrazolo[5,1-c][1,4]oxazine (Intermediate 66) instead of 3-bromo-2-(5-fluoropyridin-2-yl)-6,7-dihydro-4H-pyrazolo[5,1-c][1,4]oxazine (Intermediate 37), 4-(4,4,5,5-tetramethyl-1,3,2-dioxaborolan-2-yl)-1-((2-(trimethylsilyl)ethoxy)methyl)-1H-pyrazolo[3,4-b]pyridine (Intermediate 23) instead of 4-(4,4,5,5-tetramethyl-1,3,2-dioxaborolan-2-yl)-1-((2-(trimethylsilyl)ethoxy)methyl)-1H-pyrrolo[2,3-b]pyridine (Intermediate 21), Pd(dppf)Cl₂ instead of CataCXium® A Pd G3, and microwave irradiation at 90° C. for 1 h instead of conventional heating in Step A. MS (ESI): mass calcd. for C₁₈H₁₄ClN₅O 351.1 m/z found 352.0 [M+H]⁺. ¹H NMR (400 MHz, CDCl₃) δ 12.37 (br s, 1H), 8.59 (d, J=4.8 Hz, 1H), 7.55 (s, 1H), 7.34 (d, J=8.5 Hz, 2H), 7.21 (d, J=8.5 Hz, 2H), 6.94 (d, J=4.8 Hz, 1H), 4.86 (s, 2H), 4.42-4.31 (m, 2H), 4.30-4.19 (m, 2H).

### Example 68: 2-(4-Chlorophenyl)-3-(6-methyl-1H-pyrazolo[3,4-b]pyridin-4-yl)-6,7-dihydro-4H-pyrazolo[5,1-c][1,4]oxazine

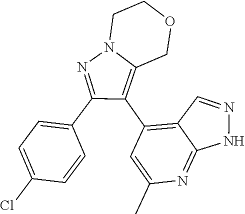 [SMILES: Cc1cc(-c2c(-c3ccc(Cl)cc3)nn3c2COCC3)c2cn[nH]c2n1]

The title compound was prepared in a manner analogous to Example 1, Steps A-B except using 3-bromo-2-(4-chlorophenyl)-6,7-dihydro-4H-pyrazolo[5,1-c][1,4]oxazine (Intermediate 66) instead of 3-bromo-2-(5-fluoropyridin-2-yl)-6,7-dihydro-4H-pyrazolo[5,1-c][1,4]oxazine (Intermediate 37), 6-Methyl-4-(4,4,5,5-tetramethyl-1,3,2-dioxaborolan-2-yl)-1-((2-(trimethylsilyl)ethoxy)methyl)-1H-pyrazolo[3,4-b]pyridine (Intermediate 26) instead of 4-(4,4,5,5-tetramethyl-1,3,2-dioxaborolan-2-yl)-1-((2-(trimethylsilyl)ethoxy)methyl)-1H-pyrrolo[2,3-b]pyridine (Intermediate 21), Pd(dppf)Cl₂ instead of CataCXium® A Pd G3, and microwave irradiation at 90° C. for 1 h instead of conventional heating in Step A. MS (ESI): mass calcd. for C₁₉H₁₆ClN₅O 365.1 m/z found 366.0 [M+H]⁺. ¹H NMR (400 MHz, CDCl₃) δ 11.54 (br s, 1H), 7.45 (s, 1H), 7.37-7.32 (m, 2H), 7.24-7.18 (m, 2H), 6.81 (s, 1H), 4.85 (s, 2H), 4.40-4.32 (m, 2H), 4.26-4.21 (m, 2H), 2.72 (s, 3H).

### Example 69: 2-(4-Fluorophenyl)-3-(1H-pyrrolo[2,3-b]pyridin-4-yl)-6,7-dihydro-4H-pyrazolo[5,1-c][1,4]oxazine

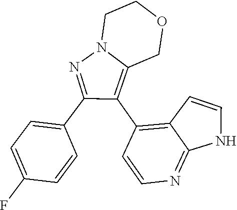 [SMILES: Fc1ccc(-c2nn3c(c2-c2ccnc4[nH]ccc24)COCC3)cc1]

To a solution of potassium phosphate tribasic (5 mg, 0.024 mmol) in H₂O (0.19 mL) was added XPhos Pd G3 (2 mg, 0.0024 mmol), 3-bromo-2-(4-fluorophenyl)-6,7-dihydro-4H-pyrazolo[5,1-c][1,4]oxazine (Intermediate 65, 7 mg, 0.024 mmol), 4-(4,4,5,5-tetramethyl-1,3,2-dioxaborolan-2-yl)-1H-pyrrolo[2,3-b]pyridine (Intermediate 21, 12 mg, 0.047 mmol), and THF (0.19 mL). The reaction vial was capped and degassed by sparging with N₂. The mixture was heated to 60° C. for 5 h. The reaction mixture was cooled, diluted with H₂O (2 mL), and extracted with EtOAc (3×2 mL). The combined organics were dried (Na₂SO₄) and filtered. Purification by chromatography (silica gel, 0-100% EtOAc/hexanes) afforded 6 mg (76%) of the title compound. MS (ESI): mass calcd. for C₁₉H₁₅FN₄O, 334.1; m/z found, 335.1 [M+H]⁺. ¹H NMR (400 MHz, CDCl₃): δ 10.48-10.23 (m, 1H), 7.43-7.27 (m, 4H), 7.06-6.83 (m, 3H), 6.27-6.09 (m, 1H), 4.83-4.75 (m, 2H), 4.40-4.30 (m, 2H), 4.29-4.18 (m, 2H).

### Example 70: 2-(4-Fluorophenyl)-3-(1H-pyrazolo[3,4-b]pyridin-4-yl)-6,7-dihydro-4H-pyrazolo[5,1-c][1,4]oxazine

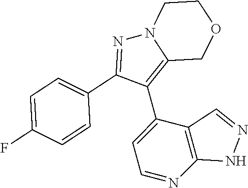 [SMILES: Fc1ccc(-c2nn3c(c2-c2ccnc4[nH]ncc24)COCC3)cc1]

The title compound was prepared in a manner analogous to Example 1, Steps A-B, except using 3-bromo-2-(4-fluorophenyl)-6,7-dihydro-4H-pyrazolo[5,1-c][1,4]oxazine (Intermediate 65) instead of 3-bromo-2-(5-fluoropyridin-2-yl)-6,7-dihydro-4H-pyrazolo[5,1-c][1,4]oxazine (Intermediate 37); and 4-(4,4,5,5-tetramethyl-1,3,2-dioxaborolan-2-yl)-1-((2-(trimethylsilyl)ethoxy)methyl)-1H-pyrazolo[3,4-b]pyridine (Intermediate 23) instead of 4-(4,4,5,5-tetramethyl-1,3,2-dioxaborolan-2-yl)-1-((2-(trimethylsilyl)ethoxy)methyl)-1H-pyrrolo[2,3-b]pyridine (Intermediate 21) and [1,1′-bis(diphenylphosphino)ferrocene]dichloropalladium(II) instead of CataCXium® A Pd G3 in Step A. MS (ESI): mass calcd. for C₁₈H₁₄FN₅O, 335.1; m/z found, 335.9 [M+H]⁺. ¹H NMR (400 MHz, DMSO-d₆) δ 8.48 (d, J=4.6 Hz, 1H), 7.39 (s, 1H), 7.34-7.27 (m, 2H), 7.14-7.06 (m, 2H), 6.99 (d, J=4.6 Hz, 1H), 4.85 (s, 2H), 4.32-4.22 (m, 2H), 4.20-4.12 (m, 2H). N—H proton is not observed.

### Example 71: 2-(4-Fluorophenyl)-3-(6-methyl-1H-pyrazolo[3,4-b]pyridin-4-yl)-6,7-dihydro-4H-pyrazolo[5,1-c][1,4]oxazine

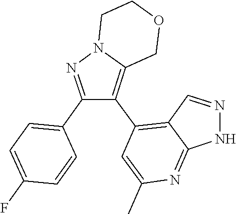 [SMILES: Cc1cc(-c2c(-c3ccc(F)cc3)nn3c2COCC3)c2cn[nH]c2n1]

The title compound was prepared in a manner analogous to Example 1, Steps A-B, except using 3-bromo-2-(4-fluorophenyl)-6,7-dihydro-4H-pyrazolo[5,1-c][1,4]oxazine (Intermediate 65) instead of 3-bromo-2-(5-fluoropyridin-2-yl)-6,7-dihydro-4H-pyrazolo[5,1-c][1,4]oxazine (Intermediate 37) and 6-methyl-4-(4,4,5,5-tetramethyl-1,3,2-dioxaborolan-2-yl)-1-((2-(trimethylsilyl)ethoxy)methyl)-1H-pyrazolo[3,4-b]pyridine (Intermediate 26) instead of 4-(4,4,5,5-tetramethyl-1,3,2-dioxaborolan-2-yl)-1-((2-(trimethylsilyl)ethoxy)methyl)-1H-pyrrolo[2,3-b]pyridine (Intermediate 21) in Step A. MS (ESI): mass calcd. for C₁₉H₁₆FN₅O, 349.1; m/z found, 349.9 [M+H]⁺. ¹H NMR (400 MHz, DMSO-d₆) δ 13.37 (br s, 1H), 7.39-7.28 (m, 2H), 7.23 (s, 1H), 7.14-7.03 (m, 2H), 6.92 (s, 1H), 4.86 (s, 2H), 4.31-4.21 (m, 2H), 4.21-4.09 (m, 2H), 2.56 (s, 3H).

### Example 72: 2-(4-Chloro-3-fluoro-phenyl)-3-(1H-pyrazolo[3,4-b]pyridin-4-yl)-6,7-dihydro-4H-pyrazolo[5,1-c][1,4]oxazine

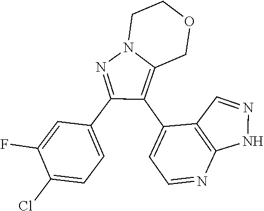 [SMILES: Fc1cc(-c2nn3c(c2-c2ccnc4[nH]ncc24)COCC3)ccc1Cl]

The title compound was prepared in a manner analogous to Example 1, Step A-B except using 3-bromo-2-(4-chloro-3-fluorophenyl)-6,7-dihydro-4H-pyrazolo[5,1-c][1,4]oxazine (Intermediate 67) instead of 3-bromo-2-(5-fluoropyridin-2-yl)-6,7-dihydro-4H-pyrazolo[5,1-c][1,4]oxazine (Intermediate 37), 4-(4,4,5,5-tetramethyl-1,3,2-dioxaborolan-2-yl)-1-((2-(trimethylsilyl)ethoxy)methyl)-1H-pyrazolo[3,4-b]pyridine (Intermediate 23) instead of 4-(4,4,5,5-tetramethyl-1,3,2-dioxaborolan-2-yl)-1-((2-(trimethylsilyl)ethoxy)methyl)-1H-pyrrolo[2,3-b]pyridine (Intermediate 21), Pd(dppf)Cl₂ instead of CataCXium® A Pd G3, and microwave irradiation at 90° C. for 1 h instead of conventional heating. MS (ESI): mass calcd. for C₁₈H₁₃ClFN₅O 369.1 m/z found 370.1 [M+H]⁺. ¹H NMR (400 MHz, DMSO-d₆) δ 13.67 (br s, 1H), 8.51 (d, J=4.5 Hz, 1H), 7.52-7.42 (m, 2H), 7.32 (d, J=10.5 Hz, 1H), 7.07-7.02 (m, 2H), 4.85 (s, 2H), 4.33-4.25 (m, 2H), 4.21-4.14 (m, 2H).

### Example 73: 2-(4-Chloro-3-fluoro-phenyl)-3-(6-methyl-1H-pyrazolo[3,4-b]pyridin-4-yl)-6,7-dihydro-4H-pyrazolo[5,1-c][1,4]oxazine

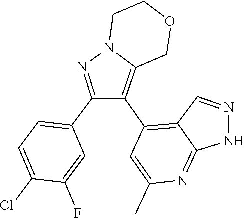 [SMILES: Cc1cc(-c2c(-c3ccc(Cl)c(F)c3)nn3c2COCC3)c2cn[nH]c2n1]

Step A: 2-(4-Chloro-3-fluorophenyl)-3-(6-methyl-1-((2-(trimethylsilyl)ethoxy)methyl)-1H-pyrazolo[3,4-b]pyridin-4-yl)-6,7-dihydro-4H-pyrazolo[5,1-c][1,4]oxazine. 3-Bromo-2-(4-chloro-3-fluorophenyl)-6,7-dihydro-4H-pyrazolo[5,1-c][1,4]oxazine (Intermediate 67, 150 mg, 0.45 mmol), 6-methyl-4-(4,4,5,5-tetramethyl-1,3,2-dioxaborolan-2-yl)-1-((2-(trimethylsilyl)ethoxy)methyl)-1H-pyrazolo[3,4-b]pyridine (Intermediate 26, 210 mg, 0.539 mmol) and Cs₂CO₃ (450 mg, 1.38 mmol) were dissolved 2-methyl-2-butanol (4 mL) and H₂O (1 mL). The resulting mixture was sparged with N₂ for 5 minutes then treated with Pd(dppf)Cl₂ (35 mg, 0.048 mmol). The mixture was microwaved at 90° C. for 1 hour then concentrated to dryness under reduced pressure. The resulting residue was purified by FCC (SiO₂, eluent: dichloromethane:methanol=1:0 to 10:1) to afford the title compound (200 mg).

Step B: 2-(4-Chloro-3-fluoro-phenyl)-3-(6-methyl-1H-pyrazolo[3,4-b]pyridin-4-yl)-6,7-dihydro-4H-pyrazolo[5,1-c][1,4]oxazine. A mixture consisting of 2-(4-chloro-3-fluorophenyl)-3-(6-methyl-1-((2-(trimethylsilyl)ethoxy)methyl)-1H-pyrazolo[3,4-b]pyridin-4-yl)-6,7-dihydro-4H-pyrazolo[5,1-c][1,4]oxazine (200 mg, 0.389 mmol) and TFA/dichloromethane (1:3, 4 mL) were stirred at room-temperature for 1 hour. The reaction mixture was concentrated to dryness under reduced pressure. The residue was basified with 2M NH₃ in MeOH (2 mL) and the resulting residue was purified by preparative HPLC Method E: to afford the title compound (31.1 mg, 21%) as a white solid. MS (ESI): mass calcd. for C₁₉H₁₅ClFN₅O 383.1; m/z found 384.0 [M+H]⁺. ¹H NMR (400 MHz, DMSO-d₆) δ 13.43 (br s, 1H), 7.48-7.41 (m, 1H), 7.36-7.28 (m, 2H), 7.08-7.02 (m, 1H), 6.96 (s, 1H), 4.86 (s, 2H), 4.33-4.23 (m, 2H), 4.21-4.12 (m, 2H), 2.58 (s, 3H).

### Example 74: 2-(5-Fluoro-2-pyridyl)-3-(4-pyridyl)-6,7-dihydro-4H-pyrazolo[5,1-c][1,4]oxazine

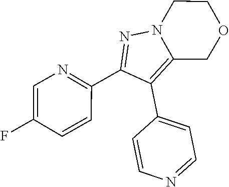 [SMILES: Fc1ccc(-c2nn3c(c2-c2ccncc2)COCC3)nc1]

The title compound was prepared in a manner analogous to Example 1 Step A, using pyridin-4-ylboronic acid instead of 4-(4,4,5,5-tetramethyl-1,3,2-dioxaborolan-2-yl)-1-((2-(trimethylsilyl)ethoxy)methyl)-1H-pyrrolo[2,3-b]pyridine (Intermediate 21). MS (ESI): mass calcd. for C₁₆H₁₃FN₄O, 296.1; m/z found, 297.1 [M+H]⁺. ¹H NMR (500 MHz, CDCl₃): δ 8.58-8.53 (m, 2H), 8.39 (d, J=2.9 Hz, 1H), 7.61 (dd, J=8.7, 4.4 Hz, 1H), 7.39 (td, J=8.4, 2.9 Hz, 1H), 7.16-7.12 (m, 2H), 4.89 (s, 2H), 4.35-4.29 (m, 2H), 4.24-4.16 (m, 2H).

### Example 75: 3-(2-(Difluoromethyl)-1H-pyrrolo[2,3-b]pyridin-4-yl)-2-(5-fluoropyridin-2-yl)-6,7-dihydro-4H-pyrazolo[5,1-c][1,4]oxazine

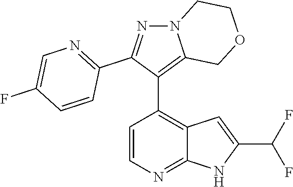 [SMILES: Fc1ccc(-c2nn3c(c2-c2ccnc4[nH]c(C(F)F)cc24)COCC3)nc1]

The title compound was prepared in a manner analogous to Example 3, Steps A-B, except using 2-(difluoromethyl)-1-(phenylsulfonyl)-4-(4,4,5,5-tetramethyl-1,3,2-dioxaborolan-2-yl)-1H-pyrrolo[2,3-b]pyridine (Intermediate 32) instead of 1-(phenylsulfonyl)-4-(4,4,5,5-tetramethyl-1,3,2-dioxaborolan-2-yl)-1H-pyrrolo[2,3-b]pyridine and 3-bromo-2-(5-fluoropyridin-2-yl)-6,7-dihydro-4H-pyrazolo[5,1-c][1,4]oxazine (Intermediate 37) instead of 3-bromo-2-(5-fluoropyridin-2-yl)-4,5,6,7-tetrahydropyrazolo[1,5-a]pyridine (Intermediate 49) in Step A. MS (ESI): mass calcd. for C₁₉H₁₄F₃N₅O, 385.1; m/z found, 386.1 [M+H]⁺. ¹H NMR (500 MHz, DMSO-d₆) δ 12.30 (s, 1H) 8.27 (d, J=4.88 Hz, 1H) 8.25 (d, J=2.88 Hz, 1H) 7.77-7.82 (m, 1H) 7.69-7.75 (m, 1H) 6.92-7.16 (m, 2H) 6.15 (d, J=2.13 Hz, 1H) 4.76 (s, 2H) 4.25-4.31 (m, 2H) 4.15-4.21 (m, 2H).

### Example 76: 2-(5-Fluoro-3-pyridyl)-3-(1H-pyrazolo[3,4-b]pyridin-4-yl)-6,7-dihydro-4H-pyrazolo[5,1-c][1,4]oxazine

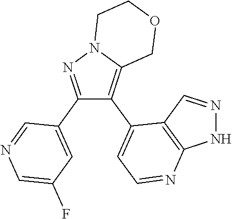 [SMILES: Fc1cncc(-c2nn3c(c2-c2ccnc4[nH]ncc24)COCC3)c1]

The title compound was prepared in a manner analogous to Example 1, Step A-B except using 3-bromo-2-(5-fluoropyridin-3-yl)-6,7-dihydro-4H-pyrazolo[5,1-c][1,4]oxazine (Intermediate 68) instead of 3-bromo-2-(5-fluoropyridin-2-yl)-6,7-dihydro-4H-pyrazolo[5,1-c][1,4]oxazine (Intermediate 37), 4-(4,4,5,5-tetramethyl-1,3,2-dioxaborolan-2-yl)-1-((2-(trimethylsilyl)ethoxy)methyl)-1H-pyrazolo[3,4-b]pyridine (Intermediate 23) instead of 4-(4,4,5,5-tetramethyl-1,3,2-dioxaborolan-2-yl)-1-((2-(trimethylsilyl)ethoxy)methyl)-1H-pyrrolo[2,3-b]pyridine (Intermediate 21) and microwave irradiation at 90° C. for 1 h instead of conventional heating. MS (ESI): mass calcd. for C₁₇H₁₃FN₆O 336.1 m/z found 337.0 [M+H]⁺. ¹H NMR (400 MHz, CDCl₃) δ 13.09 (br s, 1H), 8.62 (d, J=4.8 Hz, 1H), 8.41 (s, 1H), 8.36 (d, J=2.8 Hz, 1H), 7.60 (s, 1H), 7.57-7.50 (m, 1H), 6.96 (d, J=4.8 Hz, 1H), 4.86 (s, 2H), 4.44-4.33 (m, 2H), 4.32-4.20 (m, 2H).

### Example 77: 2-(5-Fluoro-3-pyridyl)-3-(6-methyl-1H-pyrazolo[3,4-b]pyridin-4-yl)-6,7-dihydro-4H-pyrazolo[5,1-c][1,4]oxazine

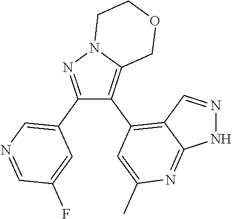 [SMILES: Cc1cc(-c2c(-c3cncc(F)c3)nn3c2COCC3)c2cn[nH]c2n1]

The title compound was prepared in a manner analogous to Example 1, Step A-B except using 3-bromo-2-(5-fluoropyridin-3-yl)-6,7-dihydro-4H-pyrazolo[5,1-c][1,4]oxazine (Intermediate 68) instead of 3-bromo-2-(5-fluoropyridin-2-yl)-6,7-dihydro-4H-pyrazolo[5,1-c][1,4]oxazine (Intermediate 37), 6-methyl-4-(4,4,5,5-tetramethyl-1,3,2-dioxaborolan-2-yl)-1-((2-(trimethylsilyl)ethoxy)methyl)-1H-pyrazolo[3,4-b]pyridine (Intermediate 26) instead of 4-(4,4,5,5-tetramethyl-1,3,2-dioxaborolan-2-yl)-1-((2-(trimethylsilyl)ethoxy)methyl)-1H-pyrrolo[2,3-b]pyridine (Intermediate 21) and microwave irradiation at 90° C. for 1 h instead of conventional heating. MS (ESI): mass calcd. for C₁₈H₁₅FN₆O 350.1 m/z found 351.0 [M+H]⁺. ¹H NMR (400 MHz, CDCl₃) δ 8.41 (s, 1H), 8.39-8.33 (m, 1H), 7.55-7.50 (m, 2H), 6.84 (s, 1H), 4.85 (s, 2H), 4.42-4.33 (m, 2H), 4.30-4.22 (m, 2H), 2.81-2.72 (m, 3H).

### Example 78: 2-(5-Fluoro-2-pyridyl)-3-(1H-pyrazolo[3,4-b]pyridin-4-yl)-6,7-dihydro-4H-pyrazolo[5,1-c][1,4]oxazine

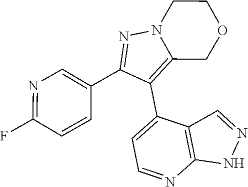 [SMILES: Fc1ccc(-c2nn3c(c2-c2ccnc4[nH]ncc24)COCC3)cn1]

The title compound was prepared in a manner analogous to Example 1, Steps A-B, using 4-(4,4,5,5-tetramethyl-1,3,2-dioxaborolan-2-yl)-1-((2-(trimethylsilyl)ethoxy)methyl)-1H-pyrazolo[3,4-b]pyridine (Intermediate 23) instead of 4-(4,4,5,5-tetramethyl-1,3,2-dioxaborolan-2-yl)-1-((2-(trimethylsilyl)ethoxy)methyl)-1H-pyrrolo[2,3-b]pyridine (Intermediate 21) in Step A. MS (ESI): mass calcd. for C₁₇H₁₃FN₆O, 336.1; m/z found, 337.2 [M+H]⁺. ¹H NMR (400 MHz, CDCl₃): δ 11.95 (s, 1H), 8.69-8.47 (m, 1H), 8.28 (s, 1H), 7.51 (d, J=23.7 Hz, 2H), 7.37-7.28 (m, 1H), 7.08-6.91 (m, 1H), 4.86 (s, 2H), 4.47-4.09 (m, 4H).

### Example 79: 2-(5-Fluoro-2-pyridyl)-3-(6-methyl-1H-pyrazolo[3,4-b]pyridin-4-yl)-6,7-dihydro-4H-pyrazolo[5,1-c][1,4]oxazine

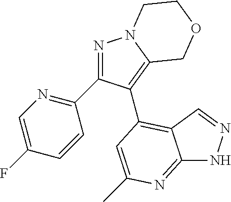 [SMILES: Cc1cc(-c2c(-c3ccc(F)cn3)nn3c2COCC3)c2cn[nH]c2n1]

The title compound was prepared in a manner analogous to Example 1, Steps A-B, using 6-methyl-4-(4,4,5,5-tetramethyl-1,3,2-dioxaborolan-2-yl)-1-((2-(trimethylsilyl)ethoxy)methyl)-1H-pyrazolo[3,4-b]pyridine (Intermediate 26) instead of 4-(4,4,5,5-tetramethyl-1,3,2-dioxaborolan-2-yl)-1-((2-(trimethylsilyl)ethoxy)methyl)-1H-pyrrolo[2,3-b]pyridine (Intermediate 21) in Step A. MS (ESI): mass calcd. for C₁₈H₁₅FN₆O, 350.1; m/z found, 351.1 [M+H]⁺. ¹H NMR (500 MHz, DMSO-d₆) δ 13.26 (s, 1H), 8.24 (d, J=2.9 Hz, 1H), 7.88-7.81 (m, 1H), 7.79-7.71 (m, 1H), 7.17 (s, 1H), 6.90 (s, 1H), 4.87 (s, 2H), 4.35-4.25 (m, 2H), 4.22-4.13 (m, 2H), 2.56 (s, 3H).

### Example 80: (R)-2-(5-Fluoropyridin-2-yl)-4-methyl-3-(1H-pyrrolo[2,3-b]pyridin-4-yl)-6,7-dihydro-4H-pyrazolo[5,1-c][1,4]oxazine

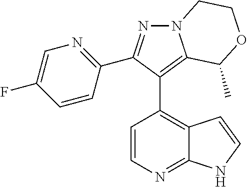 [SMILES: C[C@H]1OCCn2nc(-c3ccc(F)cn3)c(-c3ccnc4[nH]ccc34)c21]

The title compound was prepared in a manner analogous to Example 3, Step A-B except using (R)-3-bromo-2-(5-fluoropyridin-2-yl)-4-methyl-6,7-dihydro-4H-pyrazolo[5,1-c][1,4]oxazine (Intermediate 69) instead of 3-bromo-2-(5-fluoropyridin-2-yl)-4,5,6,7-tetrahydropyrazolo[1,5-a]pyridine (Intermediate 49). MS (ESI): mass calcd. for C₁₉H₁₆FN₅O, 349.1; m/z found, 350.1 [M+H]⁺. ¹H NMR (400 MHz, DMSO-d₆) δ 11.65-11.51 (m, 1H), 8.29-8.21 (m, 1H), 8.17-8.09 (m, 1H), 7.70-7.61 (m, 3H), 7.48-7.22 (m, 1H), 7.03-6.69 (m, 1H), 4.31-4.21 (m, 4H), 4.15-4.04 (m, 1H), 3.30-3.27 (m, 3H).

### Example 81: (*)-2-(4-Fluorophenyl)-6-methyl-3-(1H-pyrazolo[3,4-b]pyridin-4-yl)-6,7-dihydro-4H-pyrazolo[5,1-c][1,4]oxazine

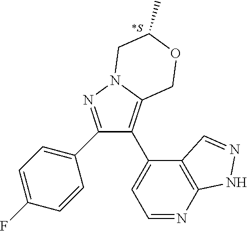 [SMILES: C[C@H]1Cn2nc(-c3ccc(F)cc3)c(-c3ccnc4[nH]ncc34)c2CO1]

The title compound was prepared in a manner analogous to Example 1, Steps A-B except using (*S)-3-bromo-2-(4-fluorophenyl)-6-methyl-6,7-dihydro-4H-pyrazolo[5,1-c][1,4]oxazine (Intermediate 72) instead of 3-bromo-2-(5-fluoropyridin-2-yl)-6,7-dihydro-4H-pyrazolo[5,1-c][1,4]oxazine (Intermediate 37), 4-(4,4,5,5-tetramethyl-1,3,2-dioxaborolan-2-yl)-1-((2-(trimethylsilyl)ethoxy)methyl)-1H-pyrazolo[3,4-b]pyridine (Intermediate 23) instead of 4-(4,4,5,5-tetramethyl-1,3,2-dioxaborolan-2-yl)-1-((2-(trimethylsilyl)ethoxy)methyl)-1H-pyrrolo[2,3-b]pyridine (Intermediate 21), Pd(dppf)Cl₂ instead of CataCXium® A Pd G3, and microwave irradiation at 95° C. for 1 h instead of conventional heating in Step A. MS (ESI): mass calcd. for C₁₉H₁₆FN₅O 349.1 m/z found 350.0 [M+H]⁺. ¹H NMR (400 MHz, CDCl₃): δ 12.49 (br s, 1H), 8.59 (d, J=4.8 Hz, 1H), 7.52 (s, 1H), 7.42-7.32 (m, 2H), 6.99-6.88 (m, 3H), 4.89 (s, 2H), 4.32 (dd, J=3.1, 12.4 Hz, 1H), 4.22-4.10 (m, 1H), 4.04-3.95 (m, 1H), 1.48 (d, J=6.0 Hz, 3H).

### Example 82: (*R)-2-(4-Fluorophenyl)-6-methyl-3-(1H-pyrazolo[3,4-b]pyridin-4-yl)-6,7-dihydro-4H-pyrazolo[5,1-c][1,4]oxazine

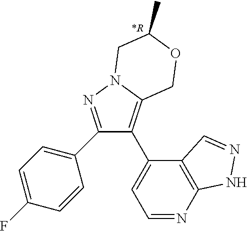 [SMILES: C[C@@H]1Cn2nc(-c3ccc(F)cc3)c(-c3ccnc4[nH]ncc34)c2CO1]

The title compound was prepared in a manner analogous to Example 1, Steps A-B except using (*R)-3-bromo-2-(4-fluorophenyl)-6-methyl-6,7-dihydro-4H-pyrazolo[5,1-c][1,4]oxazine (Intermediate 71) instead of 3-bromo-2-(5-fluoropyridin-2-yl)-6,7-dihydro-4H-pyrazolo[5,1-c][1,4]oxazine (Intermediate 37), 4-(4,4,5,5-tetramethyl-1,3,2-dioxaborolan-2-yl)-1-((2-(trimethylsilyl)ethoxy)methyl)-1H-pyrazolo[3,4-b]pyridine (Intermediate 23) instead of 4-(4,4,5,5-tetramethyl-1,3,2-dioxaborolan-2-yl)-1-((2-(trimethylsilyl)ethoxy)methyl)-1H-pyrrolo[2,3-b]pyridine (Intermediate 21), Pd(dppf)Cl₂ instead of CataCXium® A Pd G3, and microwave irradiation at 95° C. for 1 h instead of conventional heating in Step A. MS (ESI): mass calcd. for C₁₉H₁₆FN₅O 349.1 m/z found 350.0 [M+H]⁺. ¹H NMR (400 MHz, DMSO-d₆) δ 13.61 (br s, 1H), 8.48 (d, J=4.8 Hz, 1H), 7.38 (s, 1H), 7.35-7.28 (m, 2H), 7.15-7.05 (m, 2H), 7.00 (d, J=4.8 Hz, 1H), 4.98 (d, J=15.3 Hz, 1H), 4.78 (d, J=15.1 Hz, 1H), 4.35 (dd, J=3.1, 12.7 Hz, 1H), 4.22-4.13 (m, 1H), 3.94-3.84 (m, 1H), 1.34 (d, J=6.3 Hz, 3H).

### Example 83: (*S)-2-(4-Fluorophenyl)-6-methyl-3-(6-methyl-1H-pyrazolo[3,4-b]pyridin-4-yl)-6,7-dihydro-4H-pyrazolo[5,1-c][1,4]oxazine

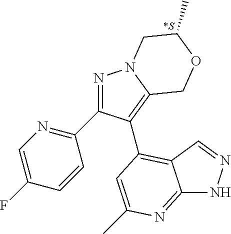 [SMILES: Cc1cc(-c2c(-c3ccc(F)cn3)nn3c2CO[C@@H](C)C3)c2cn[nH]c2n1]

The title compound was prepared in a manner analogous to Example 1, Steps A-B except using (*S)-3-bromo-2-(4-fluorophenyl)-6-methyl-6,7-dihydro-4H-pyrazolo[5,1-c][1,4]oxazine (Intermediate 72) instead of 3-bromo-2-(5-fluoropyridin-2-yl)-6,7-dihydro-4H-pyrazolo[5,1-c][1,4]oxazine (Intermediate 37), 6-Methyl-4-(4,4,5,5-tetramethyl-1,3,2-dioxaborolan-2-yl)-1-((2-(trimethylsilyl)ethoxy)methyl)-1H-pyrazolo[3,4-b]pyridine (Intermediate 26) instead of 4-(4,4,5,5-tetramethyl-1,3,2-dioxaborolan-2-yl)-1-((2-(trimethylsilyl)ethoxy)methyl)-1H-pyrrolo[2,3-b]pyridine (Intermediate 21), Pd(dppf)Cl₂ instead of CataCXium® A Pd G3, and microwave irradiation at 95° C. for 1 h instead of conventional heating in Step A. MS (ESI): mass calcd. for C₂₀H₁₈FN₅O 363.2 m/z found 364.1 [M+H]⁺. ¹H NMR (400 MHz, CDCl₃): δ 11.68 (br s, 1H), 7.42 (s, 1H), 7.40-7.34 (m, 2H), 6.97-6.89 (m, 2H), 6.80 (s, 1H), 4.88 (s, 2H), 4.31 (dd, J=3.1, 12.4 Hz, 1H), 4.22-4.09 (m, 1H), 4.05-3.91 (m, 1H), 2.72 (s, 3H), 1.48 (d, J=6.1 Hz, 3H).

### Example 84: (*R)-2-(4-Fluorophenyl)-6-methyl-3-(6-methyl-1H-pyrazolo[3,4-b]pyridin-4-yl)-6,7-dihydro-4H-pyrazolo[5,1-c][1,4]oxazine

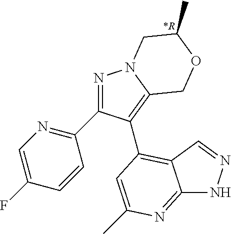 [SMILES: Cc1cc(-c2c(-c3ccc(F)cn3)nn3c2CO[C@H](C)C3)c2cn[nH]c2n1]

The title compound was prepared in a manner analogous to Example 1, Steps A-B except using (*R)-3-bromo-2-(4-fluorophenyl)-6-methyl-6,7-dihydro-4H-pyrazolo[5,1-c][1,4]oxazine (Intermediate 71) instead of 3-bromo-2-(5-fluoropyridin-2-yl)-6,7-dihydro-4H-pyrazolo[5,1-c][1,4]oxazine (Intermediate 37), 6-Methyl-4-(4,4,5,5-tetramethyl-1,3,2-dioxaborolan-2-yl)-1-((2-(trimethylsilyl)ethoxy)methyl)-1H-pyrazolo[3,4-b]pyridine (Intermediate 26) instead of 4-(4,4,5,5-tetramethyl-1,3,2-dioxaborolan-2-yl)-1-((2-(trimethylsilyl)ethoxy)methyl)-1H-pyrrolo[2,3-b]pyridine (Intermediate 21), Pd(dppf)Cl₂ instead of CataCXium® A Pd G3, and microwave irradiation at 90° C. for 1 h instead of conventional heating in Step A. MS (ESI): mass calcd. for C₂₀H₁₈FN₅O 363.2 m/z found 364.0 [M+H]⁺. ¹H NMR (400 MHz, CDCl₃): δ 11.15 (br s, 1H), 7.41 (s, 1H), 7.39-7.34 (m, 2H), 6.97-6.90 (m, 2H), 6.80 (s, 1H), 4.88 (s, 2H), 4.31 (dd, J=3.3, 12.3 Hz, 1H), 4.19-4.10 (m, 1H), 4.03-3.94 (m, 1H), 2.70 (s, 3H), 1.48 (d, J=6.3 Hz, 3H).

### Example 85: (*R)-2-(5-Fluoro-2-pyridyl)-6-methyl-3-(1H-pyrazolo[3,4-b]pyridin-4-yl)-6,7-dihydro-4H-pyrazolo[5,1-c][1,4]oxazine

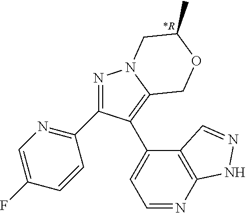 [SMILES: C[C@@H]1Cn2nc(-c3ccc(F)cn3)c(-c3ccnc4[nH]ncc34)c2CO1]

The title compound was prepared in a manner analogous to Example 1, Steps A-B except using (*R)-3-bromo-2-(5-fluoropyridin-2-yl)-6-methyl-6,7-dihydro-4H-pyrazolo[5,1-c][1,4]oxazine (Intermediate 74) instead of 3-bromo-2-(5-fluoropyridin-2-yl)-6,7-dihydro-4H-pyrazolo[5,1-c][1,4]oxazine (Intermediate 37) and (1-((2-(rimethylsilyl)ethoxy)methyl)-1H-pyrazolo[3,4-b]pyridin-4-yl)boronic acid. (Intermediate 22) instead of 4-(4,4,5,5-tetramethyl-1,3,2-dioxaborolan-2-yl)-1-((2-(trimethylsilyl)ethoxy)methyl)-1H-pyrrolo[2,3-b]pyridine (Intermediate 21) in Step A. MS (ESI): mass calcd. for C₁₈H₁₅FN₆O 350.1 m/z found 351.0 [M+H]⁺. ¹H NMR (400 MHz, CDCl₃) δ 11.67 (br s, 1H), 8.58 (d, J=4.8 Hz, 1H), 8.29 (d, J=2.8 Hz, 1H), 7.53 (dd, J=4.5, 8.8 Hz, 1H), 7.48 (s, 1H), 7.31 (dt, J=3.0, 8.4 Hz, 1H), 6.98 (d, J=4.8 Hz, 1H), 4.89 (s, 2H), 4.35 (dd, J=3.0, 12.5 Hz, 1H), 4.20-4.11 (m, 1H), 4.06-3.97 (m, 1H), 1.48 (d, J=6.0 Hz, 3H).

### Example 86: (*S)-2-(5-Fluoro-2-pyridyl)-6-methyl-3-(1H-pyrazolo[3,4-b]pyridin-4-yl)-6,7-dihydro-4H-pyrazolo[5,1-c][1,4]oxazine

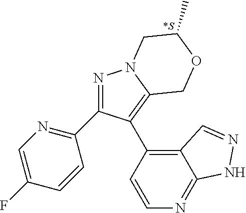 [SMILES: C[C@H]1Cn2nc(-c3ccc(F)cn3)c(-c3ccnc4[nH]ncc34)c2CO1]

The title compound was prepared in a manner analogous to Example 1, Steps A-B except using (*S)-3-bromo-2-(5-fluoropyridin-2-yl)-6-methyl-6,7-dihydro-4H-pyrazolo[5,1-c][1,4]oxazine (Intermediate 75) instead of 3-bromo-2-(5-fluoropyridin-2-yl)-6,7-dihydro-4H-pyrazolo[5,1-c][1,4]oxazine (Intermediate 37), 1-((2-(trimethylsilyl)ethoxy)methyl)-1H-pyrazolo[3,4-b]pyridin-4-yl)boronic acid. (Intermediate 22) instead of 4-(4,4,5,5-tetramethyl-1,3,2-dioxaborolan-2-yl)-1-((2-(trimethylsilyl)ethoxy)methyl)-1H-pyrrolo[2,3-b]pyridine (Intermediate 21) and microwave irradiation at 90° C. for 1 h rather than conventional heating in Step A. MS (ESI): mass calcd. for C₁₈H₁₅FN₆O 350.1 m/z found 351.0 [M+H]⁺. ¹H NMR (400 MHz, CDCl₃) δ 12.21 (br s, 1H), 8.59 (d, J=4.8 Hz, 1H), 8.29 (d, J=2.9 Hz, 1H), 7.55-7.51 (m, 1H), 7.48 (s, 1H), 7.34-7.28 (m, 1H), 6.99 (d, J=4.8 Hz, 1H), 4.89 (s, 2H), 4.39-4.32 (m, 1H), 4.21-4.11 (m, 1H), 4.06-3.97 (m, 1H), 1.48 (d, J=6.1 Hz, 3H).

### Example 87: (*R)-2-(5-Fluoro-2-pyridyl)-6-methyl-3-(6-methyl-1H-pyrazolo[3,4-b]pyridin-4-yl)-6,7-dihydro-4H-pyrazolo[5,1-c][1,4]oxazine

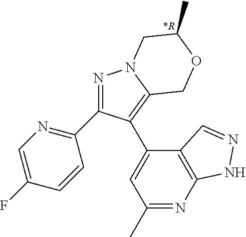 [SMILES: Cc1cc(-c2c(-c3ccc(F)cn3)nn3c2CO[C@H](C)C3)c2cn[nH]c2n1]

The title compound was prepared in a manner analogous to Example 1, Steps A-B except using (*R)-3-bromo-2-(4-fluorophenyl)-6-methyl-6,7-dihydro-4H-pyrazolo[5,1-c][1,4]oxazine (Intermediate 71) instead of 3-bromo-2-(5-fluoropyridin-2-yl)-6,7-dihydro-4H-pyrazolo[5,1-c][1,4]oxazine (Intermediate 37), 6-methyl-4-(4,4,5,5-tetramethyl-1,3,2-dioxaborolan-2-yl)-1-((2-(trimethylsilyl)ethoxy)methyl)-1H-pyrazolo[3,4-b]pyridine (Intermediate 26) instead of 4-(4,4,5,5-tetramethyl-1,3,2-dioxaborolan-2-yl)-1-((2-(trimethylsilyl)ethoxy)methyl)-1H-pyrrolo[2,3-b]pyridine (Intermediate 21), and microwave irradiation at 90° C. for 1 h instead of conventional heating in Step A. MS (ESI): mass calcd. for C₁₉H₁₇FN₆O 364.1 m/z found 365.1 [M+H]⁺. ¹H NMR (400 MHz, CDCl₃): δ 11.70 (br s, 1H), 8.30 (d, J=2.6 Hz, 1H), 7.55-7.49 (m, 1H), 7.37 (s, 1H), 7.34-7.28 (m, 1H), 6.86 (s, 1H), 4.94-4.81 (m, 2H), 4.35 (dd, J=2.8, 12.5 Hz, 1H), 4.19-4.10 (m, 1H), 4.07-3.96 (m, 1H), 2.73 (s, 3H), 1.48 (d, J=6.1 Hz, 3H).

### Example 88: (*S)-2-(5-Fluoro-2-pyridyl)-6-methyl-3-(6-methyl-1H-pyrazolo[3,4-b]pyridin-4-yl)-6,7-dihydro-4H-pyrazolo[5,1-c][1,4]oxazine

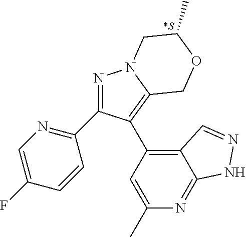 [SMILES: Cc1cc(-c2c(-c3ccc(F)cn3)nn3c2CO[C@@H](C)C3)c2cn[nH]c2n1]

The title compound was prepared in a manner analogous to Example 1, Steps A-B except using (*S)-3-bromo-2-(4-fluorophenyl)-6-methyl-6,7-dihydro-4H-pyrazolo[5,1-c][1,4]oxazine (Intermediate 72) instead of 3-bromo-2-(5-fluoropyridin-2-yl)-6,7-dihydro-4H-pyrazolo[5,1-c][1,4]oxazine (Intermediate 37), 6-Methyl-4-(4,4,5,5-tetramethyl-1,3,2-dioxaborolan-2-yl)-1-((2-(trimethylsilyl)ethoxy)methyl)-1H-pyrazolo[3,4-b]pyridine (Intermediate 26) instead of 4-(4,4,5,5-tetramethyl-1,3,2-dioxaborolan-2-yl)-1-((2-(trimethylsilyl)ethoxy)methyl)-1H-pyrrolo[2,3-b]pyridine (Intermediate 21), and microwave irradiation at 90° C. for 1 h instead of conventional heating in Step A. MS (ESI): mass calcd. for C₁₉H₁₇FN₆O 364.1 m/z found 365.0 [M+H]⁺. ¹H NMR (400 MHz, CDCl₃): δ 8.29 (d, J=2.9 Hz, 1H), 7.56-7.48 (m, 1H), 7.37 (s, 1H), 7.34-7.28 (m, 1H), 6.86 (s, 1H), 4.89 (s, 2H), 4.39-4.31 (m, 1H), 4.20-4.10 (m, 1H), 4.06-3.97 (m, 1H), 2.75 (s, 3H), 1.48 (d, J=6.1 Hz, 3H).

### Example 89: (S)-2-(5-Fluoropyridin-2-yl)-6-methyl-3-(6-methyl-1H-pyrazolo[3,4-b]pyridin-4-yl)-6,7-dihydro-4H-pyrazolo[5,1-c][1,4]oxazine

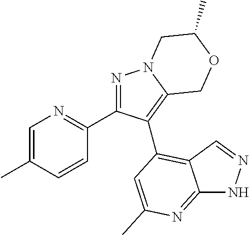 [SMILES: Cc1ccc(-c2nn3c(c2-c2cc(C)nc4[nH]ncc24)CO[C@@H](C)C3)nc1]

The title compound was prepared in a manner analogous to Example 1, Steps A-B, using 6-methyl-4-(4,4,5,5-tetramethyl-1,3,2-dioxaborolan-2-yl)-1-((2-(trimethylsilyl)ethoxy)methyl)-1H-pyrazolo[3,4-b]pyridine (Intermediate 26) instead of 4-(4,4,5,5-tetramethyl-1,3,2-dioxaborolan-2-yl)-1-((2-(trimethylsilyl)ethoxy)methyl)-1H-pyrrolo[2,3-b]pyridine (Intermediate 21) and (S)-(S)-6-methyl-6,7-dihydro-4H-[1,2,3]oxadiazolo[4,3-c][1,4]oxazin-8-ium-3-olate (Intermediate 240) instead of 3-bromo-2-(5-fluoropyridin-2-yl)-6,7-dihydro-4H-pyrazolo[5,1-c][1,4]oxazine (Intermediate 37) in Step A. MS (ESI): mass calcd. for C₁₉H₁₇FN₆O, 364.1; m/z found, 365.1 [M+H]⁺. ¹H NMR (500 MHz, CDCl₃): δ 11.74 (s, 1H), 8.29 (d, J=2.8 Hz, 1H), 7.51 (dd, J=8.8, 4.4 Hz, 1H), 7.36 (s, 1H), 7.30 (td, J=8.4, 2.9 Hz, 1H), 6.85 (s, 1H), 4.88 (d, J=3.2 Hz, 2H), 4.34 (dd, J=12.5, 3.1 Hz, 1H), 4.18-4.09 (m, 1H), 4.00 (dd, J=12.5, 10.5 Hz, 1H), 2.73 (s, 3H), 1.47 (d, J=6.1 Hz, 3H).

### Example 90: (4-(2-(4-Fluorophenyl)-7-methyl-6,7-dihydro-4H-pyrazolo[5,1-c][1,4]oxazin-3-yl)pyridin-2-yl)methanol

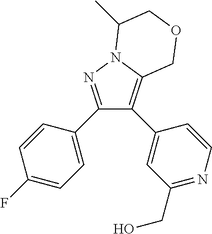 [SMILES: CC1COCc2c(-c3ccnc(CO)c3)c(-c3ccc(F)cc3)nn21]

The title compound was prepared in a manner analogous to Example 1, Step A, except using 3-bromo-2-(4-fluorophenyl)-7-methyl-6,7-dihydro-4H-pyrazolo[5,1-c][1,4]oxazine (Intermediate 76) instead of 3-bromo-2-(5-fluoropyridin-2-yl)-6,7-dihydro-4H-pyrazolo[5,1-c][1,4]oxazine (Intermediate 37) and (4-(4,4,5,5-tetramethyl-1,3,2-dioxaborolan-2-yl)pyridin-2-yl)methanol instead of 4-(4,4,5,5-tetramethyl-1,3,2-dioxaborolan-2-yl)-1-((2-(trimethylsilyl)ethoxy)methyl)-1H-pyrrolo[2,3-b]pyridine (Intermediate 21). MS (ESI): mass calcd. for C₁₉H₁₈FN₃O₂, 339.1; m/z found, 340.1 [M+H]⁺. ¹H NMR (600 MHz, DMSO-d₆) δ 8.38 (dd, J=5.1, 0.8 Hz, 1H), 7.43-7.36 (m, 2H), 7.23-7.15 (m, 3H), 6.92 (dd, J=5.1, 1.8 Hz, 1H), 5.29 (t, J=5.9 Hz, 1H), 4.97-4.77 (m, 2H), 4.51 (d, J=5.9 Hz, 2H), 4.4 (m, 1H), 4.20 (dd, J=11.9, 4.3 Hz, 1H), 3.81 (dd, J=11.9, 6.4 Hz, 1H), 1.49 (d, J=6.5 Hz, 3H).

### Example 91: (*S)-2-(4-Fluorophenyl)-7-methyl-3-(1H-pyrazolo[3,4-b]pyridin-4-yl)-6,7-dihydro-4H-pyrazolo[5,1-c][1,4]oxazine

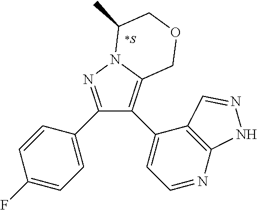 [SMILES: C[C@H]1COCc2c(-c3ccnc4[nH]ncc34)c(-c3ccc(F)cc3)nn21]

The title compound was prepared in a manner analogous to Example 1, Step A-B, except using 3-bromo-2-(4-fluorophenyl)-7-methyl-6,7-dihydro-4H-pyrazolo[5,1-c][1,4]oxazine (Intermediate 76) instead of 3-bromo-2-(5-fluoropyridin-2-yl)-6,7-dihydro-4H-pyrazolo[5,1-c][1,4]oxazine (Intermediate 37) and 4-(4,4,5,5-tetramethyl-1,3,2-dioxaborolan-2-yl)-1-((2-(trimethylsilyl)ethoxy)methyl)-1H-pyrazolo[3,4-b]pyridine (Intermediate 23) instead of 4-(4,4,5,5-tetramethyl-1,3,2-dioxaborolan-2-yl)-1-((2-(trimethylsilyl)ethoxy)methyl)-1H-pyrrolo[2,3-b]pyridine (Intermediate 21). The product of Step B was further purified by SFC over Reflect I Amylose A, 5 μm 250×21 mm, Mobile phase: 25% methanol with 0.2% triethylamine, 75% CO₂, flow rate 42 mL/min, monitor at 220 nm. The pure fractions were collected and the solvent was removed under reduced pressure to afford the title compound (143 mg). MS (ESI): mass calcd. for C₁₉H₁₆FN₅O, 349.1; m/z found, 350.2 [M+H]⁺. ¹H NMR (600 MHz, DMSO-d₆) δ 13.60 (s, 1H), 8.48 (d, J=4.7 Hz, 1H), 7.40 (d, J=1.3 Hz, 1H), 7.36-7.30 (m, 2H), 7.11 (t, J=8.9 Hz, 2H), 6.99 (d, J=4.7 Hz, 1H), 4.96-4.75 (m, 2H), 4.52-4.41 (m, 1H), 4.24 (dd, J=11.9, 4.3 Hz, 1H), 3.84 (dd, J=11.9, 6.5 Hz, 1H), 1.54 (d, J=6.5 Hz, 3H).

### Example 92: (*R)-2-(4-Fluorophenyl)-7-methyl-3-(1H-pyrazolo[3,4-b]pyridin-4-yl)-6,7-dihydro-4H-pyrazolo[5,1-c][1,4]oxazine

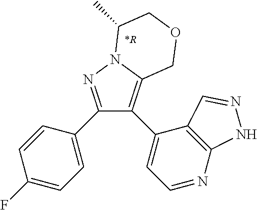 [SMILES: C[C@@H]1COCc2c(-c3ccnc4[nH]ncc34)c(-c3ccc(F)cc3)nn21]

The title compound was prepared in a manner analogous to Example 1, Step A-B, except using 3-bromo-2-(4-fluorophenyl)-7-methyl-6,7-dihydro-4H-pyrazolo[5,1-c][1,4]oxazine (Intermediate 76) instead of 3-bromo-2-(5-fluoropyridin-2-yl)-6,7-dihydro-4H-pyrazolo[5,1-c][1,4]oxazine (Intermediate 37) and 4-(4,4,5,5-tetramethyl-1,3,2-dioxaborolan-2-yl)-1-((2-(trimethylsilyl)ethoxy)methyl)-1H-pyrazolo[3,4-b]pyridine (Intermediate 23) instead of 4-(4,4,5,5-tetramethyl-1,3,2-dioxaborolan-2-yl)-1-((2-(trimethylsilyl)ethoxy)methyl)-1H-pyrrolo[2,3-b]pyridine (Intermediate 21). The product of Step B was further purified by SFC over Reflect I Amylose A, 5 μm 250×21 mm, Mobile phase: 25% methanol with 0.2% triethylamine, 75% CO₂, flow rate 42 mL/min, monitor at 220 nm. The pure fractions were collected and the solvent was removed under reduced pressure to afford the title compound (146 mg). MS (ESI): mass calcd. for MS (ESI): mass calcd. for C₁₉H₁₆FN₅O, 349.1; m/z found, 350.2 [M+H]⁺. ¹H NMR (600 MHz, DMSO-d₆) δ 13.60 (s, 1H), 8.48 (d, J=4.7 Hz, 1H), 7.40 (d, J=1.3 Hz, 1H), 7.37-7.30 (m, 2H), 7.14-7.08 (m, 2H), 6.99 (d, J=4.7 Hz, 1H), 4.96-4.75 (m, 2H), 4.52-4.41 (m, 1H), 4.24 (dd, J=11.9, 4.3 Hz, 1H), 3.84 (dd, J=11.9, 6.5 Hz, 1H), 1.54 (d, J=6.5 Hz, 2H).

### Example 93: (*S)-2-(4-Fluorophenyl)-7-methyl-3-(6-methyl-1H-pyrazolo[3,4-b]pyridin-4-yl)-6,7-dihydro-4H-pyrazolo[5,1-c][1,4]oxazine

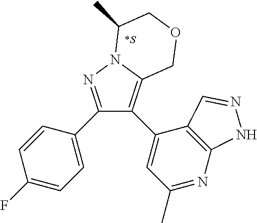 [SMILES: Cc1cc(-c2c(-c3ccc(F)cc3)nn3c2COC[C@@H]3C)c2cn[nH]c2n1]

The title compound was prepared in a manner analogous to Example 1, Step A-B, except using 3-bromo-2-(4-fluorophenyl)-7-methyl-6,7-dihydro-4H-pyrazolo[5,1-c][1,4]oxazine (Intermediate 76) instead of 3-bromo-2-(5-fluoropyridin-2-yl)-6,7-dihydro-4H-pyrazolo[5,1-c][1,4]oxazine (Intermediate 37) and 6-methyl-4-(4,4,5,5-tetramethyl-1,3,2-dioxaborolan-2-yl)-1-((2-(trimethylsilyl)ethoxy)methyl)-1H-pyrazolo[3,4-b]pyridine (Intermediate 26) instead of 4-(4,4,5,5-tetramethyl-1,3,2-dioxaborolan-2-yl)-1-((2-(trimethylsilyl)ethoxy)methyl)-1H-pyrrolo[2,3-b]pyridine (Intermediate 21). The product of Step B was further purified by SFC over Reflect I Amylose A, 5 μm 250×21 mm, Mobile phase: 25% methanol with 0.2% triethylamine, 75% CO₂, flow rate 42 mL/min, monitor at 220 nm. The pure fractions were collected and the solvent was removed under reduced pressure to afford the title compound (178 mg). MS (ESI): mass calcd. for C₂₀H₁₈FN₅O, 363.1; m/z found, 364.1 [M+H]⁺. ¹H NMR (600 MHz, DMSO-d₆) δ 13.35 (s, 1H), 7.36-7.29 (m, 2H), 7.24 (d, J=1.4 Hz, 1H), 7.09 (t, J=8.9 Hz, 2H), 6.91 (s, 1H), 4.95-4.75 (m, 2H), 4.44 (q, J=6.0 Hz, 1H), 4.25-4.18 (m, 1H), 3.88-3.78 (m, 1H), 2.56 (s, 3H), 1.53 (d, J=6.5 Hz, 3H).

### Example 94: (*R)-2-(4-Fluorophenyl)-7-methyl-3-(6-methyl-1H-pyrazolo[3,4-b]pyridin-4-yl)-6,7-dihydro-4H-pyrazolo[5,1-c][1,4]oxazine

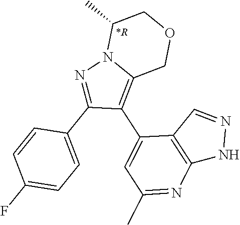 [SMILES: Cc1cc(-c2c(-c3ccc(F)cc3)nn3c2COC[C@H]3C)c2cn[nH]c2n1]

The title compound was prepared in a manner analogous to Example 1, Step A-B, except using 3-bromo-2-(4-fluorophenyl)-7-methyl-6,7-dihydro-4H-pyrazolo[5,1-c][1,4]oxazine (Intermediate 76) instead of 3-bromo-2-(5-fluoropyridin-2-yl)-6,7-dihydro-4H-pyrazolo[5,1-c][1,4]oxazine (Intermediate 37) and 6-methyl-4-(4,4,5,5-tetramethyl-1,3,2-dioxaborolan-2-yl)-1-((2-(trimethylsilyl)ethoxy)methyl)-1H-pyrazolo[3,4-b]pyridine (Intermediate 26) instead of 4-(4,4,5,5-tetramethyl-1,3,2-dioxaborolan-2-yl)-1-((2-(trimethylsilyl)ethoxy)methyl)-1H-pyrrolo[2,3-b]pyridine (Intermediate 21). The product of Step B was further purified by SFC over Reflect I Amylose A, 5 μm 250×21 mm, Mobile phase: 25% methanol with 0.2% triethylamine, 75% CO₂, flow rate 42 mL/min, monitor at 220 nm. The pure fractions were collected and the solvent was removed under reduced pressure to afford the title compound (239 mg). MS (ESI): mass calcd. for C₂₀H₁₈FN₅O, 363.1; m/z found, 364.1 [M+H]⁺. ¹H NMR (600 MHz, DMSO-d₆) δ 13.35 (s, 1H), 7.36-7.29 (m, 2H), 7.24 (d, J=1.4 Hz, 1H), 7.09 (t, J=8.9 Hz, 2H), 6.91 (s, 1H), 4.95-4.75 (m, 2H), 4.44 (q, J=6.0 Hz, 1H), 4.25-4.18 (m, 1H), 3.88-3.78 (m, 1H), 2.56 (s, 3H), 1.53 (d, J=6.5 Hz, 3H).

### Example 95: (*R)-6-Ethyl-2-(5-fluoropyridin-2-yl)-3-(1H-pyrazolo[3,4-b]pyridin-4-yl)-6,7-dihydro-4H-pyrazolo[5,1-c][1,4]oxazine

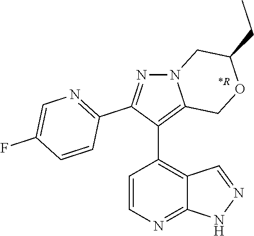 [SMILES: CC[C@@H]1Cn2nc(-c3ccc(F)cn3)c(-c3ccnc4[nH]ncc34)c2CO1]

The title compound was prepared in a manner analogous to Example 1, Steps A-B, using 3-bromo-6-ethyl-2-(5-fluoropyridin-2-yl)-6,7-dihydro-4H-pyrazolo[5,1-c][1,4]oxazine (Intermediate 77) instead of 3-bromo-2-(5-fluoropyridin-2-yl)-6,7-dihydro-4H-pyrazolo[5,1-c][1,4]oxazine (Intermediate 37) and 4-(4,4,5,5-tetramethyl-1,3,2-dioxaborolan-2-yl)-1-((2-(trimethylsilyl)ethoxy)methyl)-1H-pyrazolo[3,4-b]pyridine (Intermediate 23) instead of 4-(4,4,5,5-tetramethyl-1,3,2-dioxaborolan-2-yl)-1-((2-(trimethylsilyl)ethoxy)methyl)-1H-pyrrolo[2,3-b]pyridine (Intermediate 21). The product of Step B was further purified by SFC Method F. The pure fractions were collected and the solvent was removed under reduced pressure to afford the title compound (13.1 mg) as a white solid. LC-MS (ESI): mass calcd. for C₁₉H₁₇FN₆O 364.14 m/z found 365.1 [M+H]⁺. ¹H NMR (400 MHz, DMSO-d₆) δ 13.51 (br s, 1H), 8.44 (d, J=4.9 Hz, 1H), 8.23 (d, J=2.7 Hz, 1H), 7.88-7.84 (m, 1H), 7.81-7.75 (m, 1H), 7.31 (s, 1H), 6.99 (d, J=4.6 Hz, 1H), 5.01-4.95 (m, 1H), 4.84-4.77 (m, 1H), 4.37 (d, J=9.8 Hz, 1H), 4.00-3.91 (m, 2H), 1.73-1.66 (m, 2H), 1.02 (t, J=7.3 Hz, 3H).

### Example 96: (*S)-6-Ethyl-2-(5-fluoropyridin-2-yl)-3-(1H-pyrazolo[3,4-b]pyridin-4-yl)-6,7-dihydro-4H-pyrazolo[5,1-c][1,4]oxazine

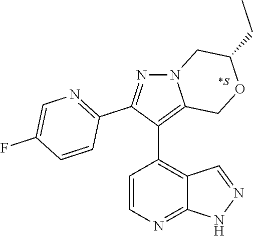 [SMILES: CC[C@H]1Cn2nc(-c3ccc(F)cn3)c(-c3ccnc4[nH]ncc34)c2CO1]

The title compound was prepared in a manner analogous to Example 1, Steps A-B, 3-bromo-6-ethyl-2-(5-fluoropyridin-2-yl)-6,7-dihydro-4H-pyrazolo[5,1-c][1,4]oxazine (Intermediate 77) instead of 3-bromo-2-(5-fluoropyridin-2-yl)-6,7-dihydro-4H-pyrazolo[5,1-c][1,4]oxazine (Intermediate 37) and 4-(4,4,5,5-tetramethyl-1,3,2-dioxaborolan-2-yl)-1-((2-(trimethylsilyl)ethoxy)methyl)-1H-pyrazolo[3,4-b]pyridine (Intermediate 23) instead of 4-(4,4,5,5-tetramethyl-1,3,2-dioxaborolan-2-yl)-1-((2-(trimethylsilyl)ethoxy)methyl)-1H-pyrrolo[2,3-b]pyridine (Intermediate 21). The product of Step B was further purified by SFC Method F. The pure fractions were collected and the solvent was removed under reduced pressure to afford the title compound (19.2 mg) as a white solid. LC-MS (ESI): mass calcd. for C₁₉H₁₇FN₆O 364.14 m/z found 365.0 [M+H]⁺. ¹H NMR (400 MHz, DMSO-d₆) δ 13.52 (br s, 1H), 8.45 (d, J=4.6 Hz, 1H), 8.24 (d, J=2.9 Hz, 1H), 7.89-7.84 (m, 1H), 7.81-7.75 (m, 1H), 7.31 (s, 1H), 6.99 (d, J=4.6 Hz, 1H), 5.01-4.96 (m, 1H), 4.84-4.78 (m, 1H), 4.38 (d, J=9.8 Hz, 1H), 4.00-3.92 (m, 2H), 1.73-1.66 (m, 2H), 1.02 (t, J=7.3 Hz, 3H).

### Example 97: (*R)-2-(5-Fluoropyridin-2-yl)-6-isopropyl-3-(1H-pyrazolo[3,4-b]pyridin-4-yl)-6,7-dihydro-4H-pyrazolo[5,1-c][1,4]oxazine

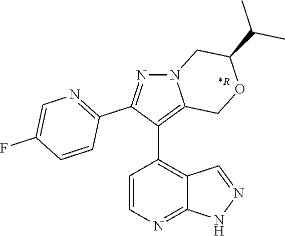 [SMILES: CC(C)[C@@H]1Cn2nc(-c3ccc(F)cn3)c(-c3ccnc4[nH]ncc34)c2CO1]

The title compound was prepared in a manner analogous to Example 1, Steps A-B, using 3-bromo-2-(5-fluoropyridin-2-yl)-6-isopropyl-6,7-dihydro-4H-pyrazolo[5,1-c][1,4]oxazine (Intermediate 78) instead of 3-bromo-2-(5-fluoropyridin-2-yl)-6,7-dihydro-4H-pyrazolo[5,1-c][1,4]oxazine (Intermediate 37) and (1-((2-(trimethylsilyl)ethoxy)methyl)-1H-pyrazolo[3,4-b]pyridin-4-yl)boronic acid (Intermediate 22) instead of 4-(4,4,5,5-tetramethyl-1,3,2-dioxaborolan-2-yl)-1-((2-(trimethylsilyl)ethoxy)methyl)-1H-pyrrolo[2,3-b]pyridine (Intermediate 21). The product of Step B was further purified by SFC Method D. The pure fractions were collected and removed under reduced pressure to afford the title compound (30.2 mg) as a white solid. LC-MS (ESI): mass calcd. for C₂₀H₁₉FN₆O 378.2 m/z found 379.1 [M+H]⁺. ¹H NMR (400 MHz, DMSO-d₆) δ 13.52 (s, 1H), 8.44 (d, J=4.8 Hz, 1H), 8.23 (d, J=2.7 Hz, 1H), 7.88-7.83 (m, 1H), 7.81-7.75 (m, 1H), 7.30 (s, 1H), 6.99 (d, J=4.6 Hz, 1H), 5.01-4.93 (m, 1H), 4.85-4.79 (m, 1H), 4.36 (dd, J=2.9, 12.6 Hz, 1H), 4.04 (t, J=11.6 Hz, 1H), 3.83-3.75 (m, 1H), 1.96-1.84 (m, 1H), 1.03 (d, J=6.7 Hz, 3H), 1.00 (d, J=6.8 Hz, 3H).

### Example 98: (*S)-2-(5-Fluoropyridin-2-yl)-6-isopropyl-3-(1H-pyrazolo[3,4-b]pyridin-4-yl)-6,7-dihydro-4H-pyrazolo[5,1-c][1,4]oxazine

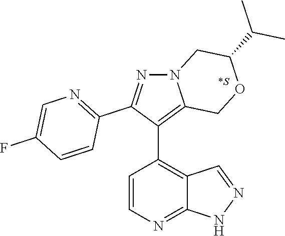 [SMILES: CC(C)[C@H]1Cn2nc(-c3ccc(F)cn3)c(-c3ccnc4[nH]ncc34)c2CO1]

The title compound was prepared in a manner analogous to Example 1, A-B, except using 3-bromo-2-(5-fluoropyridin-2-yl)-6-isopropyl-6,7-dihydro-4H-pyrazolo[5,1-c][1,4]oxazine (Intermediate 78) instead of 3-bromo-2-(5-fluoropyridin-2-yl)-6,7-dihydro-4H-pyrazolo[5,1-c][1,4]oxazine (Intermediate 37) and (1-((2-(trimethylsilyl)ethoxy)methyl)-1H-pyrazolo[3,4-b]pyridin-4-yl)boronic acid (Intermediate 22) instead of 4-(4,4,5,5-tetramethyl-1,3,2-dioxaborolan-2-yl)-1-((2-(trimethylsilyl)ethoxy)methyl)-1H-pyrrolo[2,3-b]pyridine (Intermediate 21). The product of Step B was further purified by SFC Method D. The pure fractions were collected and removed under reduced pressure to afford the title compound (40 mg, crude) which was subjected to an additional preparative HPLC Method F. The product was suspended in water (15 mL), the mixture frozen using dry ice/EtOH, and then lyophilized to dryness to afford the title compound (25.3 mg) as a white solid. LC-MS (ESI): mass calcd. for C₂₀H₁₉FN₆O 378.16 m/z found 379.1 [M+H]⁺. ¹H NMR (400 MHz, DMSO-d₆) δ 8.44 (d, J=4.6 Hz, 1H), 8.23 (d, J=2.7 Hz, 1H), 7.88-7.84 (m, 1H), 7.81-7.75 (m, 1H), 7.31 (s, 1H), 6.99 (d, J=4.8 Hz, 1H), 5.00-4.95 (m, 1H), 4.85-4.80 (m, 1H), 4.37 (dd, J=3.0, 12.5 Hz, 1H), 4.04 (t, J=11.7 Hz, 1H), 3.83-3.76 (m, 1H), 1.96-1.86 (m, 1H), 1.03 (d, J=6.7 Hz, 3H), 1.00 (d, J=6.8 Hz, 3H).

### Example 99: 2-(4-Fluorophenyl)-3-(1H-pyrazolo[3,4-b]pyridin-4-yl)-6-(trifluoromethyl)-6,7-dihydro-4H-pyrazolo[5,1-c][1,4]oxazine

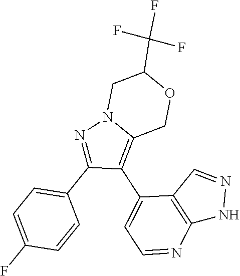 [SMILES: Fc1ccc(-c2nn3c(c2-c2ccnc4[nH]ncc24)COC(C(F)(F)F)C3)cc1]

The title compound was prepared in a manner analogous to Example 1, Step A-B except using 3-bromo-2-(4-fluorophenyl)-6-(trifluoromethyl)-6,7-dihydro-4H-pyrazolo[5,1-c][1,4]oxazine (Intermediate 79) instead of 3-bromo-2-(5-fluoropyridin-2-yl)-6,7-dihydro-4H-pyrazolo[5,1-c][1,4]oxazine (Intermediate 37) and 4-(4,4,5,5-tetramethyl-1,3,2-dioxaborolan-2-yl)-1-((2-(trimethylsilyl)ethoxy)methyl)-1H-pyrazolo[3,4-b]pyridine (Intermediate 23) instead of 4-(4,4,5,5-tetramethyl-1,3,2-dioxaborolan-2-yl)-1-((2-(trimethylsilyl)ethoxy)methyl)-1H-pyrrolo[2,3-b]pyridine (Intermediate 21). MS (ESI): mass calcd. for C₁₉H₁₃F₄N₅O, 403.1; m/z found, 404.1 [M+H]⁺. ¹H NMR (500 MHz, CD₃OD) δ 8.51 (d, J=4.8 Hz, 1H), 7.41 (s, 1H), 7.40-7.33 (m, 2H), 7.06 (d, J=4.8 Hz, 1H), 7.04-6.96 (m, 2H), 5.14 (d, J=15.2 Hz, 1H), 5.06 (d, J=15.2 Hz, 1H), 4.87 (ddd, J=10.3, 6.0, 4.0 Hz, 1H), 4.57 (dd, J=12.3, 3.8 Hz, 1H), 4.35 (dd, J=12.3, 10.6 Hz, 1H).

### Example 100: (S)-2-(4-Fluorophenyl)-3-(1H-pyrazolo[3,4-b]pyridin-4-yl)-6-(trifluoromethyl)-6,7-dihydro-4H-pyrazolo[5,1-c][1,4]oxazine

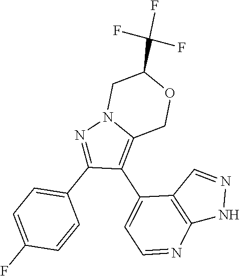 [SMILES: Fc1ccc(-c2nn3c(c2-c2ccnc4[nH]ncc24)CO[C@H](C(F)(F)F)C3)cc1]

The title compound was prepared by via SFC purification of the racemic product of Example 99 (Stationary phase: Reflect I Cellulose B 5 μm 250×21 mm, Mobile phase: 35% methanol with 0.2% triethylamine, 65% CO₂, Flow rate 42 mL/min, monitor at 220 nm). MS (ESI): mass calcd. for C₁₉H₁₃F₄N₅O, 403.1; m/z found, 404.1 [M+H]⁺.

### Example 101: (R)-2-(4-Fluorophenyl)-3-(1H-pyrazolo[3,4-b]pyridin-4-yl)-6-(trifluoromethyl)-6,7-dihydro-4H-pyrazolo[5,1-c][1,4]oxazine

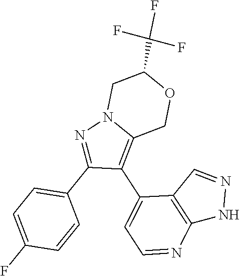 [SMILES: Fc1ccc(-c2nn3c(c2-c2ccnc4[nH]ncc24)CO[C@@H](C(F)(F)F)C3)cc1]

The title compound was prepared via SFC purification of the racemic product of Example 99 (Stationary phase: Reflect I Cellulose B 5 μm 250×21 mm, Mobile phase: 35% methanol with 0.2% triethylamine, 65% CO₂, Flow rate 42 mL/min, monitor at 220 nm). MS (ESI): mass calcd. for C₁₉H₁₃F₄N₅O, 403.1; m/z found, 404.1 [M+H]⁺.

### Example 102: 2-(5-Fluoropyridin-2-yl)-3-(1H-pyrazolo[3,4-b]pyridin-4-yl)-6-(trifluoromethyl)-6,7-dihydro-4H-pyrazolo[5,1-c][1,4]oxazine

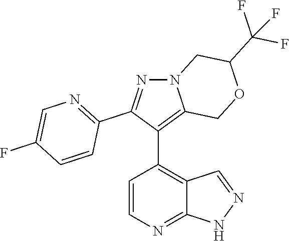 [SMILES: Fc1ccc(-c2nn3c(c2-c2ccnc4[nH]ncc24)COC(C(F)(F)F)C3)nc1]

The title compound was prepared in a manner analogous to Example 1, Steps A-B, except using 3-bromo-2-(5-fluoropyridin-2-yl)-6-(trifluoromethyl)-6,7-dihydro-4H-pyrazolo[5,1-c][1,4]oxazine (Intermediate 80) instead of 3-bromo-2-(5-fluoropyridin-2-yl)-6,7-dihydro-4H-pyrazolo[5,1-c][1,4]oxazine (Intermediate 37) and 4-(4,4,5,5-tetramethyl-1,3,2-dioxaborolan-2-yl)-1-((2-(trimethylsilyl)ethoxy)methyl)-1H-pyrazolo[3,4-b]pyridine (Intermediate 23) instead of 4-(4,4,5,5-tetramethyl-1,3,2-dioxaborolan-2-yl)-1-((2-(trimethylsilyl)ethoxy)methyl)-1H-pyrrolo[2,3-b]pyridine (Intermediate 21) in Step A. MS (ESI): mass calcd. for C₁₈H₁₂F₄N₆O, 404.1; m/z found, 405.2 [M+H]⁺. ¹H NMR (400 MHz, DMSO-d₆) δ 13.55 (s, 1H), 8.47 (d, J=4.75 Hz, 1H), 8.25 (d, J=2.88 Hz, 1H), 7.89-7.76 (m, 2H), 7.33 (s, 1H), 7.01 (d, J=4.75 Hz, 1H), 5.21-5.14 (m, 1H), 5.14-5.00 (m, 2H), 4.64 (dd, J=12.32, 3.69 Hz, 1H), 4.43-4.33 (m, 1H).

### Example 103: 6-(Fluoromethyl)-2-(4-fluorophenyl)-3-(1H-pyrazolo[3,4-b]pyridin-5-yl)-6,7-dihydro-4H-pyrazolo[5,1-c][1,4]oxazine

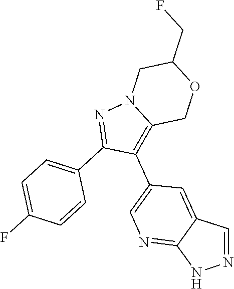 [SMILES: FCC1Cn2nc(-c3ccc(F)cc3)c(-c3cnc4[nH]ncc4c3)c2CO1]

The title compound was prepared in a manner analogous to Example 1, Step A-B except using 3-bromo-6-(fluoromethyl)-2-(4-fluorophenyl)-6,7-dihydro-4H-pyrazolo[5,1-c][1,4]oxazine (Intermediate 81) instead of 3-bromo-2-(5-fluoropyridin-2-yl)-6,7-dihydro-4H-pyrazolo[5,1-c][1,4]oxazine (Intermediate 37) and 4-(4,4,5,5-tetramethyl-1,3,2-dioxaborolan-2-yl)-1-((2-(trimethylsilyl)ethoxy)methyl)-1H-pyrazolo[3,4-b]pyridine (Intermediate 23) instead of 4-(4,4,5,5-tetramethyl-1,3,2-dioxaborolan-2-yl)-1-((2-(trimethylsilyl)ethoxy)methyl)-1H-pyrrolo[2,3-b]pyridine (Intermediate 21) in Step A. MS (ESI): mass calcd. for C₁₉H₁₅F₂N₅O, 367.1; m/z found, 368.2 [M+H]⁺. ¹H NMR (400 MHz, CD₃OD) δ 8.52 (d, J=4.8 Hz, 1H), 7.48-7.31 (m, 3H), 7.08 (d, J=4.8 Hz, 1H), 7.06-6.96 (m, 2H), 5.07 (dt, J=15.4, 1.0 Hz, 1H), 4.96 (d, J=15.3 Hz, 1H), 4.81-4.71 (m, 1H), 4.70-4.60 (m, 1H), 4.48-4.34 (m, 2H), 4.18 (dd, J=12.6, 11.3 Hz, 1H).

### Example 104: (*S)-6-(Fluoromethyl)-2-(4-fluorophenyl)-3-(1H-pyrazolo[3,4-b]pyridin-4-yl)-6,7-dihydro-4H-pyrazolo[5,1-c][1,4]oxazine

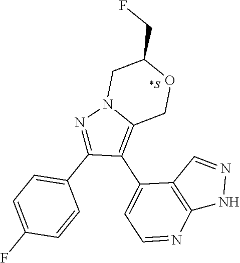 [SMILES: FC[C@@H]1Cn2nc(-c3ccc(F)cc3)c(-c3ccnc4[nH]ncc34)c2CO1]

The title compound was prepared via SFC purification of the racemic product of Example 103 (Stationary phase: Reflect I Cellulose J 5 μm 250×21 mm, Mobile phase: 35% methanol with 0.2% triethylamine, 65% CO₂, Flow rate 42 mL/min, monitor at 220 nm). MS (ESI): mass calcd. for C₁₉H₁₅F₂N₅O, 367.1; m/z found, 368.1 [M+H]⁺.

### Example 105: (*R)-(Fluoromethyl)-2-(4-fluorophenyl)-3-(1H-pyrazolo[3,4-b]pyridin-4-yl)-6,7-dihydro-4H-pyrazolo[5,1-c][1,4]oxazine

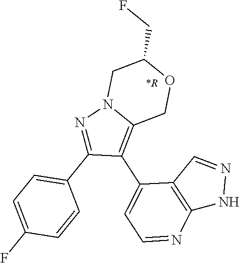 [SMILES: FC[C@H]1Cn2nc(-c3ccc(F)cc3)c(-c3ccnc4[nH]ncc34)c2CO1]

The title compound was prepared via SFC purification of the racemic product of Example 103 (Stationary phase: Reflect I Cellulose J 5 μm 250×21 mm, Mobile phase: 35% methanol with 0.2% triethylamine, 65% CO₂, Flow rate 42 mL/min, monitor at 220 nm). MS (ESI): mass calcd. for C₁₉H₁₅F₂N₅O, 367.1; m/z found, 368.2 [M+H]⁺.

### Example 106: 2′-(4-Fluorophenyl)-3′-(1H-pyrazolo[3,4-b]pyridin-4-yl)-4′H,6′H-spiro[cyclopropane-1,7′-pyrazolo[5,1-c][1,4]oxazine]

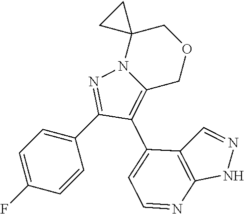 [SMILES: Fc1ccc(-c2nn3c(c2-c2ccnc4[nH]ncc24)COCC32CC2)cc1]

The title compound was prepared in a manner analogous to Example 1, Steps A-B, except using 3′-bromo-2′-(4-fluorophenyl)-4′H,6′H-spiro[cyclopropane-1,7′-pyrazolo[5,1-c][1,4]oxazine](Intermediate 82) instead of 3-bromo-2-(5-fluoropyridin-2-yl)-6,7-dihydro-4H-pyrazolo[5,1-c][1,4]oxazine (Intermediate 37) and 4-(4,4,5,5-tetramethyl-1,3,2-dioxaborolan-2-yl)-1-((2-(trimethylsilyl)ethoxy)methyl)-1H-pyrazolo[3,4-b]pyridine (Intermediate 23) instead of 4-(4,4,5,5-tetramethyl-1,3,2-dioxaborolan-2-yl)-1-((2-(trimethylsilyl)ethoxy)methyl)-1H-pyrrolo[2,3-b]pyridine (Intermediate 21) in Step A. LC-MS (ESI): mass calcd. for C₂₀H₁₆FN₅O 361.13 m/z found 362.1 [M+H]⁺. ¹H NMR (400 MHz, DMSO-d₆) δ 13.61 (br s, 1H), 8.48 (d, J=4.8 Hz, 1H), 7.42 (s, 1H), 7.31-7.26 (m, 2H), 7.12-7.07 (m, 2H), 7.00 (d, J=4.6 Hz, 1H), 4.96 (s, 2H), 4.10 (s, 2H), 1.57-1.51 (m, 2H), 1.12-1.07 (m, 2H).

### Example 107: 2′-(4-Fluorophenyl)-3′-(6-methyl-1H-pyrazolo[3,4-b]pyridin-4-yl)-4′H,6′H-spiro[cyclopropane-1,7-pyrazolo[5,1-c][1,4]oxazine]

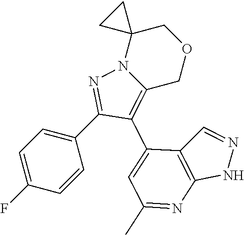 [SMILES: Cc1cc(-c2c(-c3ccc(F)cc3)nn3c2COCC32CC2)c2cn[nH]c2n1]

The title compound was prepared in a manner analogous to Example 1, Step A-B, except using 3′-bromo-2′-(4-fluorophenyl)-4′H,6′H-spiro[cyclopropane-1,7′-pyrazolo[5,1-c][1,4]oxazine](Intermediate 82) instead of 3-bromo-2-(5-fluoropyridin-2-yl)-6,7-dihydro-4H-pyrazolo[5,1-c][1,4]oxazine (Intermediate 37), 6-methyl-4-(4,4,5,5-tetramethyl-1,3,2-dioxaborolan-2-yl)-1-((2-(trimethylsilyl)ethoxy)methyl)-1H-pyrazolo[3,4-b]pyridine (Intermediate 26) instead of 4-(4,4,5,5-tetramethyl-1,3,2-dioxaborolan-2-yl)-1-((2-(trimethylsilyl)ethoxy)methyl)-1H-pyrrolo[2,3-b]pyridine (Intermediate 21). LC-MS (ESI): mass calcd. for C₂₁H₁₈FN₅O 375.15 m/z found 376.2 [M+H]⁺. ¹H NMR (400 MHz, DMSO-d₆) δ 13.37 (br s, 1H), 7.31-7.26 (m, 2H), 7.25 (s, 1H), 7.12-7.05 (m, 2H), 6.92 (s, 1H), 4.97 (s, 2H), 4.09 (s, 2H), 2.56 (s, 3H), 1.56-1.51 (m, 2H), 1.11-1.07 (m, 2H).

### Example 108: (*R)-6-Cyclopropyl-2-(5-fluoropyridin-2-yl)-3-(1H-pyrazolo[3,4-b]pyridin-4-yl)-6,7-dihydro-4H-pyrazolo[5,1-c][1,4]oxazine

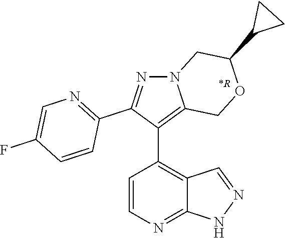 [SMILES: Fc1ccc(-c2nn3c(c2-c2ccnc4[nH]ncc24)CO[C@H](C2CC2)C3)nc1]

The title compound was prepared in a manner analogous to Example 1, Steps A-B, except using 3-bromo-6-cyclopropyl-2-(5-fluoropyridin-2-yl)-6,7-dihydro-4H-pyrazolo[5,1-c][1,4]oxazine (Intermediate 83) instead of 3-bromo-2-(5-fluoropyridin-2-yl)-6,7-dihydro-4H-pyrazolo[5,1-c][1,4]oxazine (Intermediate 37) and 4-(4,4,5,5-tetramethyl-1,3,2-dioxaborolan-2-yl)-1-((2-(trimethylsilyl)ethoxy)methyl)-1H-pyrazolo[3,4-b]pyridine (Intermediate 23) instead of 4-(4,4,5,5-tetramethyl-1,3,2-dioxaborolan-2-yl)-1-((2-(trimethylsilyl)ethoxy)methyl)-1H-pyrrolo[2,3-b]pyridine (Intermediate 21). The product of Step B was further purified by SFC Method F. The pure fractions were collected and the volatiles were removed under reduced pressure. The product was suspended in water (10 mL), the mixture frozen using dry ice/ethanol, and then lyophilized to dryness to afford the title compound (50 mg, crude) as a white solid. This material was subjected to an additional preparative HPLC Method G: to afford pure product. The product was suspended in water (10 mL), the mixture frozen using dry ice/ethanol, and then lyophilized to dryness to yield the title compound (27.5 mg, 25%) as a light yellow solid. LC-MS (ESI): mass calcd. for C₂₀H₁₇FN₆O 376.14 m/z, found 377.2 [M+H]⁺. ¹H NMR (400 MHz, CDCl₃): δ 11.58 (br s, 1H), 8.57 (d, J=4.4 Hz, 1H), 8.28 (d, J=2.8 Hz, 1H), 7.53 (dd, J=4.4, 8.8 Hz, 1H), 7.46 (s, 1H), 7.35-7.28 (m, 1H), 6.97 (d, J=4.0 Hz, 1H), 4.99-4.77 (m, 2H), 4.44 (br dd, J=3.2, 12.8 Hz, 1H), 4.26-4.17 (m, 1H), 3.43-3.31 (m, 1H), 1.20-1.07 (m, 1H), 0.78-0.67 (m, 2H), 0.61-0.52 (m, 1H), 0.49-0.38 (m, 1H).

### Example 109: (*S)-6-Cyclopropyl-2-(5-fluoropyridin-2-yl)-3-(1H-pyrazolo[3,4-b]pyridin-4-yl)-6,7-dihydro-4H-pyrazolo[5,1-c][1,4]oxazine

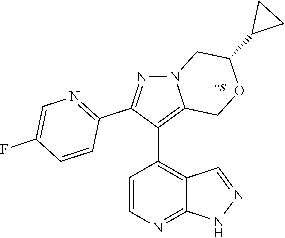 [SMILES: Fc1ccc(-c2nn3c(c2-c2ccnc4[nH]ncc24)CO[C@@H](C2CC2)C3)nc1]

The title compound was prepared in a manner analogous to Example 1, Steps A-B, except using 3-bromo-6-cyclopropyl-2-(5-fluoropyridin-2-yl)-6,7-dihydro-4H-pyrazolo[5,1-c][1,4]oxazine (Intermediate 83) instead of 3-bromo-2-(5-fluoropyridin-2-yl)-6,7-dihydro-4H-pyrazolo[5,1-c][1,4]oxazine (Intermediate 37) and 4-(4,4,5,5-tetramethyl-1,3,2-dioxaborolan-2-yl)-1-((2-(trimethylsilyl)ethoxy)methyl)-1H-pyrazolo[3,4-b]pyridine (Intermediate 23) instead of 4-(4,4,5,5-tetramethyl-1,3,2-dioxaborolan-2-yl)-1-((2-(trimethylsilyl)ethoxy)methyl)-1H-pyrrolo[2,3-b]pyridine (Intermediate 21). The product of Step B was further purified by SFC Method F. The pure fractions were collected and the volatiles were removed under reduced pressure. The product was suspended in water (10 mL), the mixture frozen using dry ice/ethanol, and then lyophilized to dryness to afford the title compound (36.9 mg, 32%) as a white solid. LC-MS (ESI): mass calcd. for C₂₀H₁₇FN₆O 376.14 m/z, found 377.1 [M+H]⁺. ¹H NMR (400 MHz, CDCl₃): δ 11.49 (br s, 1H), 8.57 (d, J=4.4 Hz, 1H), 8.28 (d, J=2.8 Hz, 1H), 7.53 (dd, J=4.4, 8.8 Hz, 1H), 7.45 (s, 1H), 7.34-7.27 (m, 1H), 6.96 (d, J=4.8 Hz, 1H), 4.97-4.79 (m, 2H), 4.43 (dd, J=3.2, 12.8 Hz, 1H), 4.21 (dd, J=10.4, 12.8 Hz, 1H), 3.42-3.31 (m, 1H), 1.19-1.08 (m, 1H), 0.77-0.66 (m, 2H), 0.62-0.50 (m, 1H), 0.49-0.37 (m, 1H).

### Example 110: (*R)-6-Cyclobutyl-2-(5-fluoropyridin-2-yl)-3-(1H-pyrazolo[3,4-b]pyridin-4-yl)-6,7-dihydro-4H-pyrazolo[5,1-c][1,4]oxazine

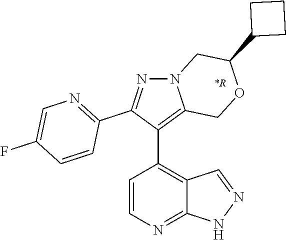 [SMILES: Fc1ccc(-c2nn3c(c2-c2ccnc4[nH]ncc24)CO[C@H](C2CCC2)C3)nc1]

The title compound was prepared in a manner analogous to Example 1, Steps A-B, except using 3-bromo-6-cyclobutyl-2-(5-fluoropyridin-2-yl)-6,7-dihydro-4H-pyrazolo[5,1-c][1,4]oxazine (Intermediate 84) instead of 3-bromo-2-(5-fluoropyridin-2-yl)-6,7-dihydro-4H-pyrazolo[5,1-c][1,4]oxazine (Intermediate 37) and (1-((2-(trimethylsilyl)ethoxy)methyl)-1H-pyrazolo[3,4-b]pyridin-4-yl)boronic acid (Intermediate 22) instead of 4-(4,4,5,5-tetramethyl-1,3,2-dioxaborolan-2-yl)-1-((2-(trimethylsilyl)ethoxy)methyl)-1H-pyrrolo[2,3-b]pyridine (Intermediate 21) in Step A. The product of Step B was further purified by SFC Method D. The pure fractions were collected and the volatiles were removed under reduced pressure. The product was suspended in water (10 mL), the mixture frozen using dry ice/ethanol, and then lyophilized to dryness to afford (63 mg, 40%) as a white solid. LC-MS (ESI): mass calcd. for C₂₁H₁₉FN₆O 390.16 m/z, found 391.2 [M+H]⁺. ¹H NMR (400 MHz, CDCl₃): δ 11.18 (br s, 1H), 8.56 (d, J=4.8 Hz, 1H), 8.27 (d, J=2.8 Hz, 1H), 7.54 (dd, J=4.4, 8.8 Hz, 1H), 7.46 (s, 1H), 7.31 (dt, J=2.8, 8.4 Hz, 1H), 6.97 (d, J=4.8 Hz, 1H), 4.95-4.83 (m, 2H), 4.33-4.24 (m, 1H), 3.97-3.88 (m, 2H), 2.69-2.57 (m, 1H), 2.15-1.90 (m, 6H).

### Example 111: (*S)-6-Cyclobutyl-2-(5-fluoropyridin-2-yl)-3-(1H-pyrazolo[3,4-b]pyridin-4-yl)-6,7-dihydro-4H-pyrazolo[5,1-c][1,4]oxazine

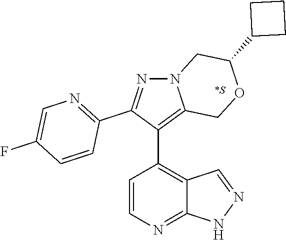 [SMILES: Fc1ccc(-c2nn3c(c2-c2ccnc4[nH]ncc24)CO[C@@H](C2CCC2)C3)nc1]

The title compound was prepared in a manner analogous to Example 1, except using 3-bromo-6-cyclobutyl-2-(5-fluoropyridin-2-yl)-6,7-dihydro-4H-pyrazolo[5,1-c][1,4]oxazine (Intermediate 84) instead of 3-bromo-2-(5-fluoropyridin-2-yl)-6,7-dihydro-4H-pyrazolo[5,1-c][1,4]oxazine (Intermediate 37) and (1-((2-(trimethylsilyl)ethoxy)methyl)-1H-pyrazolo[3,4-b]pyridin-4-yl)boronic acid (Intermediate 22) instead of 4-(4,4,5,5-tetramethyl-1,3,2-dioxaborolan-2-yl)-1-((2-(trimethylsilyl)ethoxy)methyl)-1H-pyrrolo[2,3-b]pyridine (Intermediate 21). The product of Step B was further purified by SFC Method D. The pure fractions were collected and the volatiles were removed under reduced pressure. The product was suspended in water (10 mL), the mixture frozen using dry ice/ethanol, and then lyophilized to dryness to afford (63 mg, 40%) as a white solid. LC-MS (ESI): mass calcd. for C₂₁H₁₉FN₆O 390.16 m/z, found 391.2 [M+H]⁺. ¹H NMR (400 MHz, CDCl₃): δ 11.66 (br s, 1H), 8.57 (d, J=4.8 Hz, 1H), 8.27 (d, J=2.8 Hz, 1H), 7.54 (dd, J=4.4, 8.8 Hz, 1H), 7.47 (s, 1H), 7.31 (dt, J=2.8, 8.4 Hz, 1H), 6.98 (d, J=4.8 Hz, 1H), 4.95-4.81 (m, 2H), 4.34-4.24 (m, 1H), 3.98-3.87 (m, 2H), 2.69-2.57 (m, 1H), 2.16-1.88 (m, 6H).

### Example 112: (6*R,7*S)-2-(4-Fluorophenyl)-6,7-dimethyl-3-(1H-pyrazolo[3,4-b]pyridin-4-yl)-6,7-dihydro-4H-pyrazolo[5,1-c][1,4]oxazine

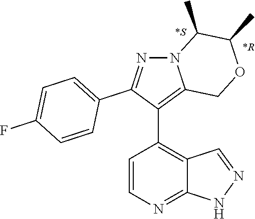 [SMILES: C[C@H]1OCc2c(-c3ccnc4[nH]ncc34)c(-c3ccc(F)cc3)nn2[C@H]1C]

Step A: (6*R,7*S)-2-(4-Fluorophenyl)-6,7-dimethyl-3-(1-((2-(trimethylsilyl)ethoxy)methyl)-1H-pyrazolo[3,4-b]pyridin-4-yl)-6,7-dihydro-4H-pyrazolo[5,1-c][1,4]oxazine and (6*S,7*R)-2-(4-fluorophenyl)-6,7-dimethyl-3-(1-((2-(trimethylsilyl)ethoxy)methyl)-1H-pyrazolo[3,4-b]pyridin-4-yl)-6,7-dihydro-4H-pyrazolo[5,1-c][1,4]oxazine. The title compounds were prepared in a manner analogous to Example 1, Step A, except using: (Racemic) cis-3-bromo-2-(4-fluorophenyl)-6,7-dimethyl-6,7-dihydro-4H-pyrazolo[5,1-c][1,4]oxazine (Intermediate 87) instead of 3-bromo-2-(5-fluoropyridin-2-yl)-6,7-dihydro-4H-pyrazolo[5,1-c][1,4]oxazine (Intermediate 37) and 4-(4,4,5,5-tetramethyl-1,3,2-dioxaborolan-2-yl)-1-((2-(trimethylsilyl)ethoxy)methyl)-1H-pyrazolo[3,4-b]pyridine (Intermediate 23) instead of 4-(4,4,5,5-tetramethyl-1,3,2-dioxaborolan-2-yl)-1-((2-(trimethylsilyl)ethoxy)methyl)-1H-pyrrolo[2,3-b]pyridine (Intermediate 21) in Step A. (Racemic) cis-2-(4-Fluorophenyl)-6,7-dimethyl-3-(1-((2-(trimethylsilyl)ethoxy)methyl)-1H-pyrazolo[3,4-b]pyridin-4-yl)-6,7-dihydro-4H-pyrazolo[5,1-c][1,4]oxazine was purified by SFC Method E. The pure fractions were collected and the volatiles were removed under reduced pressure. The product was suspended in water (10 mL), the mixture frozen using dry ice/EtOH, and then lyophilized to dryness to afford (6*R,7*S)-2-(4-Fluorophenyl)-6,7-dimethyl-3-(1-((2-(trimethylsilyl)ethoxy)methyl)-1H-pyrazolo[3,4-b]pyridin-4-yl)-6,7-dihydro-4H-pyrazolo[5,1-c][1,4]oxazine (300 mg, 43%) and (6*S,7*R)-2-(4-fluorophenyl)-6,7-dimethyl-3-(1-((2-(trimethylsilyl)ethoxy)methyl)-1H-pyrazolo[3,4-b]pyridin-4-yl)-6,7-dihydro-4H-pyrazolo[5,1-c][1,4]oxazine (300 mg, 46%).

Step B: (6*R,7*S)-2-(4-Fluorophenyl)-6,7-dimethyl-3-(1H-pyrazolo[3,4-b]pyridin-4-yl)-6,7-dihydro-4H-pyrazolo[5,1-c][1,4]oxazine. The title compound was prepared in a manner analogous to Example 1, Step B, using (6*R,7*S)-2-(4-fluorophenyl)-6,7-dimethyl-3-(1-((2-(trimethylsilyl)ethoxy)methyl)-1H-pyrazolo[3,4-b]pyridin-4-yl)-6,7-dihydro-4H-pyrazolo[5,1-c][1,4]oxazine. LC-MS (ESI): mass calcd. for C₂₀H₁₈FN₅O 363.2 m/z found 364.0 [M+H]⁺. ¹H NMR (400 MHz, DMSO-d₆) δ 13.59 (s, 1H), 8.47 (d, J=4.8 Hz, 1H), 7.39 (s, 1H), 7.35-7.27 (m, 2H), 7.13-7.05 (m, 2H), 7.02 (d, J=4.8 Hz, 1H), 4.99-4.90 (m, 1H), 4.85-4.76 (m, 1H), 4.45-4.36 (m, 1H), 4.27-4.18 (m, 1H), 2.07 (s, 1H), 1.39 (d, J=6.6 Hz, 3H), 1.27 (d, J=6.4 Hz, 3H).

### Example 113: (6*S,7*R)-2-(4-Fluorophenyl)-6,7-dimethyl-3-(1H-pyrazolo[3,4-b]pyridin-4-yl)-6,7-dihydro-4H-pyrazolo[5,1-c][1,4]oxazine

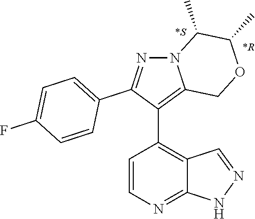 [SMILES: C[C@@H]1OCc2c(-c3ccnc4[nH]ncc34)c(-c3ccc(F)cc3)nn2[C@@H]1C]

The title compound was prepared in a manner analogous to Example 1, Step B, except using (6*S,7*R)-2-(4-fluorophenyl)-6,7-dimethyl-3-(1-((2-(trimethylsilyl)ethoxy)methyl)-1H-pyrazolo[3,4-b]pyridin-4-yl)-6,7-dihydro-4H-pyrazolo[5,1-c][1,4]oxazine (Example 112, Step A). MS (ESI): mass calcd. for C₂₀H₁₈FN₅O 363.2 m/z found 364.0 [M+H]⁺. ¹H NMR (400 MHz, DMSO-d₆) δ 13.60 (br s, 1H), 8.47 (d, J=4.8 Hz, 1H), 7.39 (s, 1H), 7.35-7.28 (m, 2H), 7.14-7.06 (m, 2H), 7.02 (d, J=4.8 Hz, 1H), 4.99-4.91 (m, 1H), 4.84-4.76 (m, 1H), 4.45-4.36 (m, 1H), 4.28-4.18 (m, 1H), 1.39 (d, J=6.6 Hz, 3H), 1.27 (d, J=6.3 Hz, 3H).

### Example 114: (6*R,7*R)-2-(4-Fluorophenyl)-6,7-dimethyl-3-(1H-pyrazolo[3,4-b]pyridin-4-yl)-6,7-dihydro-4H-pyrazolo[5,1-c][1,4]oxazine

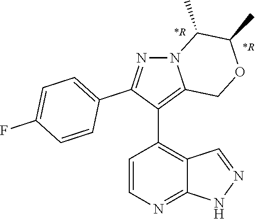 [SMILES: C[C@@H]1[C@@H](C)OCc2c(-c3ccnc4[nH]ncc34)c(-c3ccc(F)cc3)nn21]

The title compound was prepared in a manner analogous to Example 1, Steps A-B except using (Racemic) trans-3-bromo-2-(4-fluorophenyl)-6,7-dimethyl-6,7-dihydro-4H-pyrazolo[5,1-c][1,4]oxazine (Intermediate 88) instead of 3-bromo-2-(5-fluoropyridin-2-yl)-6,7-dihydro-4H-pyrazolo[5,1-c][1,4]oxazine (Intermediate 37) and 4-(4,4,5,5-tetramethyl-1,3,2-dioxaborolan-2-yl)-1-((2-(trimethylsilyl)ethoxy)methyl)-1H-pyrazolo[3,4-b]pyridine (Intermediate 23) instead of 4-(4,4,5,5-tetramethyl-1,3,2-dioxaborolan-2-yl)-1-((2-(trimethylsilyl)ethoxy)methyl)-1H-pyrrolo[2,3-b]pyridine (Intermediate 21) and microwave irradiation at 90° C. for 2 h rather than conventional heating in Step A. Purification by SFC Method D afforded (6*R,7*R)-2-(4-fluorophenyl)-6,7-dimethyl-3-(1H-pyrazolo[3,4-b]pyridin-4-yl)-6,7-dihydro-4H-pyrazolo[5,1-c][1,4]oxazine. MS (ESI): mass calcd. for C₂₀H₁₈FN₅O 363.2 m/z found 364.1 [M+H]⁺. ¹H NMR (400 MHz, DMSO-d₆) δ 8.48-13.61 (br s, 1H), 8.48 (d, J=4.6 Hz, 1H), 7.38 (s, 1H), 7.36-7.28 (m, 2H), 7.15-7.05 (m, 2H), 6.96 (d, J=4.8 Hz, 1H), 5.01 (d, J=15.0 Hz, 1H), 4.70 (d, J=15.0 Hz, 1H), 4.13-3.98 (m, 1H), 3.91-3.76 (m, 1H), 1.59 (d, J=6.3 Hz, 3H), 1.36 (d, J=6.2 Hz, 3H).

### Example 115: (6*S,7*S)-2-(4-Fluorophenyl)-6,7-dimethyl-3-(1H-pyrazolo[3,4-b]pyridin-4-yl)-6,7-dihydro-4H-pyrazolo[5,1-c][1,4]oxazine

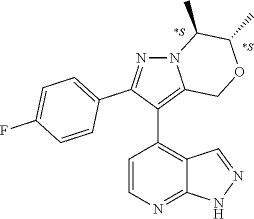 [SMILES: C[C@@H]1OCc2c(-c3ccnc4[nH]ncc34)c(-c3ccc(F)cc3)nn2[C@H]1C]

The title compound was isolated from Example 114, by SFC Method D and SFC Method B; afforded the title compound. LC-MS (ESI): mass calcd. for C₂₀H₁₈FN₅O 363.15 m/z found 364.0 [M+H]⁺. ¹H NMR (400 MHz, DMSO-d₆) δ 13.61 (s, 1H), 8.48 (d, J=4.6 Hz, 1H), 7.38 (s, 1H), 7.36-7.28 (m, 2H), 7.14-7.07 (m, 2H), 6.96 (d, J=4.8 Hz, 1H), 5.01 (d, J=15.0 Hz, 1H), 4.71 (d, J=15.0 Hz, 1H), 4.13-3.98 (m, 1H), 3.91-3.77 (m, 1H), 1.59 (d, J=6.4 Hz, 3H), 1.37 (d, J=6.1 Hz, 3H).

### Example 116: (6*R,7*S)-2-(5-Fluoropyridin-2-yl)-6,7-dimethyl-3-(1H-pyrazolo[3,4-b]pyridin-4-yl)-6,7-dihydro-4H-pyrazolo[5,1-c][1,4]oxazine

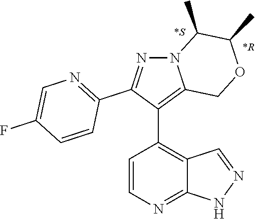 [SMILES: C[C@H]1OCc2c(-c3ccnc4[nH]ncc34)c(-c3ccc(F)cn3)nn2[C@H]1C]

The title compound was prepared in a manner analogous to Example 1, Steps A-B, except using (6*R,7*S)-3-bromo-2-(5-fluoropyridin-2-yl)-6,7-dimethyl-6,7-dihydro-4H-pyrazolo[5,1-c][1,4]oxazine (Intermediate 90) instead of 3-bromo-2-(5-fluoropyridin-2-yl)-6,7-dihydro-4H-pyrazolo[5,1-c][1,4]oxazine (Intermediate 37) and 4-(4,4,5,5-tetramethyl-1,3,2-dioxaborolan-2-yl)-1-((2-(trimethylsilyl)ethoxy)methyl)-1H-pyrazolo[3,4-b]pyridine (Intermediate 23) instead of 4-(4,4,5,5-tetramethyl-1,3,2-dioxaborolan-2-yl)-1-((2-(trimethylsilyl)ethoxy)methyl)-1H-pyrrolo[2,3-b]pyridine (Intermediate 21) and also microwave irradiation at 80° C. for 1 h in Step A. LC-MS (ESI): mass calcd. for C₁₉H₁₇FN₆O 364.14 m/z found 365.0 [M+H]⁺. ¹H NMR (400 MHz, DMSO-d₆) δ 13.52 (s, 1H), 8.44 (d, J=4.63 Hz, 1H), 8.16-8.31 (m, 1H), 7.82-7.90 (m, 1H), 7.71-7.81 (m, 1H), 7.31 (s, 1H), 7.01 (d, J=4.63 Hz, 1H), 4.74-5.04 (m, 2H), 4.38-4.51 (m, 1H), 4.19-4.31 (m, 1H), 1.24-1.44 (m, 6H).

### Example 117: (6*S,7*R)-2-(5-Fluoropyridin-2-yl)-6,7-dimethyl-3-(1H-pyrazolo[3,4-b]pyridin-4-yl)-6,7-dihydro-4H-pyrazolo[5,1-c][1,4]oxazine

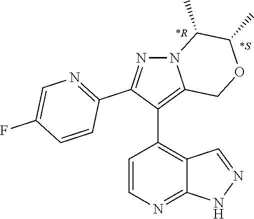 [SMILES: C[C@@H]1OCc2c(-c3ccnc4[nH]ncc34)c(-c3ccc(F)cn3)nn2[C@@H]1C]

The title compound was prepared in a manner analogous to Example 1, Steps A-B, except using (6*S,7*R)-3-bromo-2-(5-fluoropyridin-2-yl)-6,7-dimethyl-6,7-dihydro-4H-pyrazolo[5,1-c][1,4]oxazine (Intermediate 91), instead of 3-bromo-2-(5-fluoropyridin-2-yl)-6,7-dihydro-4H-pyrazolo[5,1-c][1,4]oxazine (Intermediate 37) and 4-(4,4,5,5-tetramethyl-1,3,2-dioxaborolan-2-yl)-1-((2-(trimethylsilyl)ethoxy)methyl)-1H-pyrazolo[3,4-b]pyridine (Intermediate 23) instead of 4-(4,4,5,5-tetramethyl-1,3,2-dioxaborolan-2-yl)-1-((2-(trimethylsilyl)ethoxy)methyl)-1H-pyrrolo[2,3-b]pyridine (Intermediate 21) and also microwave irradiation at 90° C. for 1 h in Step A. LC-MS (ESI): mass calcd. for C₁₉H₁₇FN₆O 364.14 m/z found 365.0 [M+H]⁺. ¹H NMR (400 MHz, DMSO-d₆) δ 13.52 (br s, 1H), 8.44 (d, J=4.8 Hz, 1H), 8.24 (d, J=2.9 Hz, 1H), 7.90-7.82 (m, 1H), 7.81-7.73 (m, 1H), 7.31 (s, 1H), 7.01 (d, J=4.8 Hz, 1H), 5.05-4.91 (m, 1H), 4.81 (d, J=15.3 Hz, 1H), 4.51-4.38 (m, 1H), 4.32-4.19 (m, 1H), 1.40 (d, J=6.7 Hz, 3H), 1.28 (d, J=6.4 Hz, 3H).

### Example 118: (6*R,7*R)-2-(5-Fluoropyridin-2-yl)-6,7-dimethyl-3-(1H-pyrazolo[3,4-b]pyridin-4-yl)-6,7-dihydro-4H-pyrazolo[5,1-c][1,4]oxazine

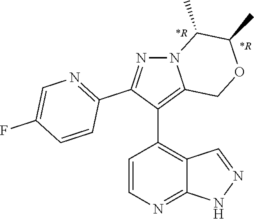 [SMILES: C[C@@H]1[C@@H](C)OCc2c(-c3ccnc4[nH]ncc34)c(-c3ccc(F)cn3)nn21]

The title compound was prepared in a manner analogous to Example 1 except using (Racemic) trans-3-bromo-2-(5-fluoropyridin-2-yl)-6,7-dimethyl-6,7-dihydro-4H-pyrazolo[5,1-c][1,4]oxazine (Intermediate 93) instead of 3-bromo-2-(5-fluoropyridin-2-yl)-6,7-dihydro-4H-pyrazolo[5,1-c][1,4]oxazine (Intermediate 37), and 4-(4,4,5,5-tetramethyl-1,3,2-dioxaborolan-2-yl)-1-((2-(trimethylsilyl)ethoxy)methyl)-1H-pyrazolo[3,4-b]pyridine (Intermediate 23) instead of 4-(4,4,5,5-tetramethyl-1,3,2-dioxaborolan-2-yl)-1-((2-(trimethylsilyl)ethoxy)methyl)-1H-pyrrolo[2,3-b]pyridine (Intermediate 21) and microwave irradiation at 80° C. for 1 h rather than conventional heating. The product from Step B was further purified by SFC Method D. The pure fractions were collected and the volatiles were removed under reduced pressure. The product was suspended in water (10 mL), the mixture frozen using dry ice/acetone, and then lyophilized to dryness to afford the title compound (87 mg, 43%) as a white solid. MS (ESI): mass calcd. for C₁₉H₁₇FN₆O 364.1 m/z found 365.0 [M+H]⁺. ¹H NMR (400 MHz, DMSO-d₆) δ 13.53 (s, 1H), 8.45 (d, J=4.77 Hz, 1H), 8.25 (d, J=2.76 Hz, 1H), 7.82-7.90 (m, 1H), 7.74-7.81 (m, 1H), 7.30 (s, 1H), 6.96 (d, J=4.77 Hz, 1H), 5.02 (d, J=15.06 Hz, 1H), 4.72 (d, J=15.06 Hz, 1H), 4.04-4.12 (m, 1H), 3.81-3.90 (m, 1H), 1.61 (d, J=6.53 Hz, 3H), 1.37 (d, J=6.27 Hz, 3H).

### Example 119: (6*S,7*S)-2-(5-Fluoropyridin-2-yl)-6,7-dimethyl-3-(1H-pyrazolo[3,4-b]pyridin-4-yl)-6,7-dihydro-4H-pyrazolo[5,1-c][1,4]oxazine

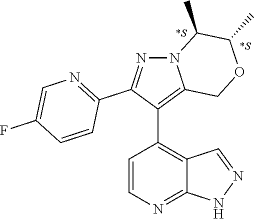 [SMILES: C[C@@H]1OCc2c(-c3ccnc4[nH]ncc34)c(-c3ccc(F)cn3)nn2[C@H]1C]

The title compound was prepared in a manner analogous to Example 1 except using (Racemic) trans-3-bromo-2-(5-fluoropyridin-2-yl)-6,7-dimethyl-6,7-dihydro-4H-pyrazolo[5,1-c][1,4]oxazine (Intermediate 93) instead of 3-bromo-2-(5-fluoropyridin-2-yl)-6,7-dihydro-4H-pyrazolo[5,1-c][1,4]oxazine (Intermediate 37) and 4-(4,4,5,5-tetramethyl-1,3,2-dioxaborolan-2-yl)-1-((2-(trimethylsilyl)ethoxy)methyl)-1H-pyrazolo[3,4-b]pyridine (Intermediate 23) instead of 4-(4,4,5,5-tetramethyl-1,3,2-dioxaborolan-2-yl)-1-((2-(trimethylsilyl)ethoxy)methyl)-1H-pyrrolo[2,3-b]pyridine (Intermediate 21) and microwave irradiation at 80° C. for 1 h rather than conventional heating. The product from Step B was further purified by SFC Method D. The pure fractions were collected and the volatiles were removed under reduced pressure. The product was suspended in water (10 mL), the mixture frozen using dry ice/acetone, and then lyophilized to dryness to afford the title compound (89 mg, 44%) as a white solid. MS (ESI): mass calcd. for C₁₉H₁₇FN₆O 364.1 m/z found 365.0 [M+H]⁺. ¹H NMR (400 MHz, DMSO-d₆) δ 13.53 (s, 1H), 8.44 (d, J=4.77 Hz, 1H), 8.25 (d, J=2.76 Hz, 1H), 7.83-7.89 (m, 1H), 7.74-7.81 (m, 1H), 7.30 (s, 1H), 6.96 (d, J=4.77 Hz, 1H), 4.98-5.07 (m, 1H), 4.68-4.76 (m, 1H), 4.03-4.12 (m, 1H), 3.81-3.89 (m, 1H), 1.60 (d, J=6.27 Hz, 3H), 1.37 (d, J=6.27 Hz, 3H).

### Example 120: 2-(4-Fluorophenyl)-7,7-dimethyl-3-(1H-pyrazolo[3,4-b]pyridin-4-yl)-4,6-dihydropyrazolo[5,1-c][1,4]oxazine

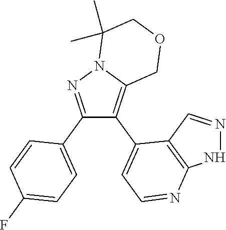 [SMILES: CC1(C)COCc2c(-c3ccnc4[nH]ncc34)c(-c3ccc(F)cc3)nn21]

The title compound was prepared in a manner analogous to Example 1, Step A-B using 3-bromo-2-(4-fluorophenyl)-7,7-dimethyl-6,7-dihydro-4H-pyrazolo[5,1-c][1,4]oxazine (Intermediate 94) instead of 3-bromo-2-(5-fluoropyridin-2-yl)-6,7-dihydro-4H-pyrazolo[5,1-c][1,4]oxazine (Intermediate 37), 4-(4,4,5,5-tetramethyl-1,3,2-dioxaborolan-2-yl)-1-((2-(trimethylsilyl)ethoxy)methyl)-1H-pyrazolo[3,4-b]pyridine (Intermediate 23) instead of 4-(4,4,5,5-tetramethyl-1,3,2-dioxaborolan-2-yl)-1-((2-(trimethylsilyl)ethoxy)methyl)-1H-pyrrolo[2,3-b]pyridine (Intermediate 21), Pd(dppf)Cl₂ instead of CataCXium® A Pd G3 and also microwave irradiation at 90° C. for 1 h rather than conventional heating in Step A. MS (ESI): mass calcd. for C₂₀H₁₈FN₅O 363.1 m/z found 364.0 [M+H]⁺. ¹H NMR (400 MHz, CDCl₃) δ 12.47 (br s, 1H), 8.58 (d, J=4.8 Hz, 1H), 7.51 (s, 1H), 7.41-7.35 (m, 2H), 6.96-6.90 (m, 3H), 4.86 (s, 2H), 3.91 (s, 2H), 1.68 (s, 6H).

### Example 121: 2-(4-Fluorophenyl)-7,7-dimethyl-3-(6-methyl-1H-pyrazolo[3,4-b]pyridin-4-yl)-4,6-dihydropyrazolo[5,1-c][1,4]oxazine

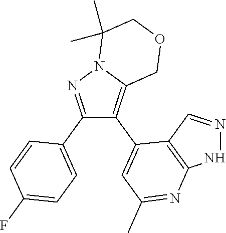 [SMILES: Cc1cc(-c2c(-c3ccc(F)cc3)nn3c2COCC3(C)C)c2cn[nH]c2n1]

The title compound was prepared in a manner analogous to Example 1, Step A-B using 3-bromo-2-(4-fluorophenyl)-7,7-dimethyl-6,7-dihydro-4H-pyrazolo[5,1-c][1,4]oxazine (Intermediate 94) instead of 3-bromo-2-(5-fluoropyridin-2-yl)-6,7-dihydro-4H-pyrazolo[5,1-c][1,4]oxazine (Intermediate 37), 6-methyl-4-(4,4,5,5-tetramethyl-1,3,2-dioxaborolan-2-yl)-1-((2-(trimethylsilyl)ethoxy)methyl)-1H-pyrazolo[3,4-b]pyridine (Intermediate 26) instead of 4-(4,4,5,5-tetramethyl-1,3,2-dioxaborolan-2-yl)-1-((2-(trimethylsilyl)ethoxy)methyl)-1H-pyrrolo[2,3-b]pyridine (Intermediate 21), Pd(dppf)Cl₂ instead of CataCXium® A Pd G3 and also microwave irradiation at 90° C. for 1 h rather than conventional heating in Step A. LC-MS (ESI): mass calcd. for C₂₁H₂₀FN₅O 377.2 m/z found 378.1 [M+H]⁺. ¹H NMR (400 MHz, CDCl₃) δ 12.58 (br s, 1H), 7.42-7.35 (m, 3H), 6.96-6.89 (m, 2H), 6.80 (s, 1H), 4.86 (s, 2H), 3.91 (s, 2H), 2.75 (s, 3H), 1.68 (s, 6H).

### Example 122: 2-(5-Fluoro-2-pyridyl)-7,7-dimethyl-3-(1H-pyrazolo[3,4-b]pyridin-4-yl)-4,6-dihydropyrazolo[5,1-c][1,4]oxazine

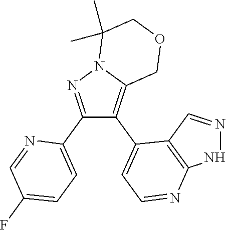 [SMILES: CC1(C)COCc2c(-c3ccnc4[nH]ncc34)c(-c3ccc(F)cn3)nn21]

The title compound was prepared in a manner analogous to Example 1, Step A-B using 3-bromo-2-(5-fluoropyridin-2-yl)-7,7-dimethyl-6,7-dihydro-4H-pyrazolo[5,1-c][1,4]oxazine (Intermediate 95) instead of 3-bromo-2-(5-fluoropyridin-2-yl)-6,7-dihydro-4H-pyrazolo[5,1-c][1,4]oxazine (Intermediate 37), (1-((2-(trimethylsilyl)ethoxy)methyl)-1H-pyrazolo[3,4-b]pyridin-4-yl)boronic acid (Intermediate 22) instead of 4-(4,4,5,5-tetramethyl-1,3,2-dioxaborolan-2-yl)-1-((2-(trimethylsilyl)ethoxy)methyl)-1H-pyrrolo[2,3-b]pyridine (Intermediate 21) and also microwave irradiation at 90° C. for 1 h rather than conventional heating in Step A. LC-MS (ESI): mass calcd. for C₁₉H₁₇FN₆O 364.14 m/z found 365.1 [M+H]⁺. ¹H NMR (400 MHz, DMSO-d₆) δ 13.53 (s, 1H), 8.43 (d, J=4.9 Hz, 1H), 8.25 (d, J=2.9 Hz, 1H), 7.87-7.73 (m, 2H), 7.31 (s, 1H), 6.97 (d, J=4.9 Hz, 1H), 4.87 (s, 2H), 3.94 (s, 2H), 1.57 (s, 6H).

### Example 123: 2-(5-Fluoro-2-pyridyl)-7,7-dimethyl-3-(6-methyl-1H-pyrazolo[3,4-b]pyridin-4-yl)-4,6-dihydropyrazolo[5,1-c][1,4]oxazine

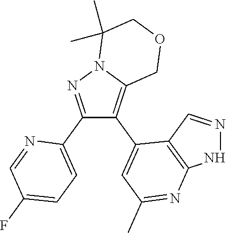 [SMILES: Cc1cc(-c2c(-c3ccc(F)cn3)nn3c2COCC3(C)C)c2cn[nH]c2n1]

The title compound was prepared in a manner analogous to Example 1, Step A-B using 3-bromo-2-(5-fluoropyridin-2-yl)-7,7-dimethyl-6,7-dihydro-4H-pyrazolo[5,1-c][1,4]oxazine (Intermediate 95) instead of 3-bromo-2-(5-fluoropyridin-2-yl)-6,7-dihydro-4H-pyrazolo[5,1-c][1,4]oxazine (Intermediate 37), 6-methyl-4-(4,4,5,5-tetramethyl-1,3,2-dioxaborolan-2-yl)-1-((2-(trimethylsilyl)ethoxy)methyl)-1H-pyrazolo[3,4-b]pyridine (Intermediate 26) instead of 4-(4,4,5,5-tetramethyl-1,3,2-dioxaborolan-2-yl)-1-((2-(trimethylsilyl)ethoxy)methyl)-1H-pyrrolo[2,3-b]pyridine (Intermediate 21) and also microwave irradiation at 90° C. for 1 h rather than conventional heating in Step A. LC-MS (ESI): mass calcd. for C₂₀H₁₉FN₆O 378.2 m/z found 379.1 [M+H]⁺. ¹H NMR (400 MHz, DMSO-d₆) δ 13.29 (s, 1H), 8.26 (d, J=2.6 Hz, 1H), 7.87-7.71 (m, 2H), 7.15 (s, 1H), 6.88 (s, 1H), 4.88 (s, 2H), 3.93 (s, 2H), 2.55 (s, 3H), 1.56 (s, 6H).

### Example 124: 6,6-Dimethyl-3-(1H-pyrazolo[3,4-b]pyridin-4-yl)-2-(thiazol-4-yl)-6,7-dihydro-4H-pyrazolo[5,1-c][1,4]oxazine

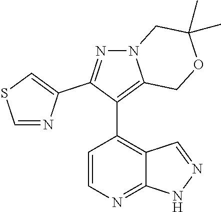 [SMILES: CC1(C)Cn2nc(-c3cscn3)c(-c3ccnc4[nH]ncc34)c2CO1]

The title compound was prepared in a manner analogous to Example 1, Step A-B except using 3-bromo-6,6-dimethyl-2-(thiazol-4-yl)-6,7-dihydro-4H-pyrazolo[5,1-c][1,4]oxazine (Intermediate 96) instead of 3-bromo-2-(5-fluoropyridin-2-yl)-6,7-dihydro-4H-pyrazolo[5,1-c][1,4]oxazine (Intermediate 37) and 4-(4,4,5,5-tetramethyl-1,3,2-dioxaborolan-2-yl)-1-((2-(trimethylsilyl)ethoxy)methyl)-1H-pyrazolo[3,4-b]pyridine (Intermediate 23) instead of 4-(4,4,5,5-tetramethyl-1,3,2-dioxaborolan-2-yl)-1-((2-(trimethylsilyl)ethoxy)methyl)-1H-pyrrolo[2,3-b]pyridine (Intermediate 21) in Step A. MS (ESI): mass calcd. for C₁₇H₁₆N₆O_{S}, 352.1; m/z found, 353.2 [M+H]⁺. ¹H NMR (400 MHz, DMSO-d₆) δ 13.50 (s, 1H), 8.90 (d, J=2.00 Hz, 1H), 8.46 (d, J=4.75 Hz, 1H), 7.81 (d, J=2.00 Hz, 1H), 7.29 (s, 1H), 7.09 (d, J=4.75 Hz, 1H), 4.87 (s, 2H), 4.10 (s, 2H), 1.38 (s, 6H).

### Example 125: 6,6-Dimethyl-3-(6-methyl-1H-pyrazolo[3,4-b]pyridin-4-yl)-2-(thiazol-4-yl)-6,7-dihydro-4H-pyrazolo[5,1-c][1,4]oxazine

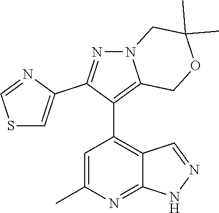 [SMILES: Cc1cc(-c2c(-c3cscn3)nn3c2COC(C)(C)C3)c2cn[nH]c2n1]

The title compound was prepared in a manner analogous to Example 1, Step A-B except using 3-bromo-6,6-dimethyl-2-(thiazol-4-yl)-6,7-dihydro-4H-pyrazolo[5,1-c][1,4]oxazine (Intermediate 96) instead of 3-bromo-2-(5-fluoropyridin-2-yl)-6,7-dihydro-4H-pyrazolo[5,1-c][1,4]oxazine (Intermediate 37) and 6-methyl-4-(4,4,5,5-tetramethyl-1,3,2-dioxaborolan-2-yl)-1-((2-(trimethylsilyl)ethoxy)methyl)-1H-pyrazolo[3,4-b]pyridine (Intermediate 26) instead of 4-(4,4,5,5-tetramethyl-1,3,2-dioxaborolan-2-yl)-1-((2-(trimethylsilyl)ethoxy)methyl)-1H-pyrrolo[2,3-b]pyridine (Intermediate 21) in step A. MS (ESI): mass calcd. for C₁₈H₁₈N₆OS, 366.1; m/z found, 367.2 [M+H]⁺. ¹H NMR (500 MHz, CD₃OD) δ 8.90 (d, J=2.0 Hz, 1H), 7.59 (d, J=2.0 Hz, 1H), 7.32 (s, 1H), 7.07 (s, 1H), 4.97 (s, 2H), 4.16 (s, 2H), 2.69 (s, 3H), 1.48 (s, 6H).

### Example 126: 6,6-Dimethyl-2-(oxazol-5-yl)-3-(1H-pyrazolo[3,4-b]pyridin-4-yl)-6,7-dihydro-4H-pyrazolo[5,1-c][1,4]oxazine

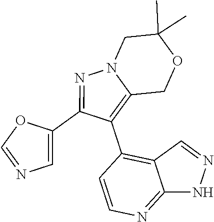 [SMILES: CC1(C)Cn2nc(-c3cnco3)c(-c3ccnc4[nH]ncc34)c2CO1]

The title compound was prepared in a manner analogous to Example 1, Step A-B except using 3-bromo-6,6-dimethyl-2-(oxazol-5-yl)-6,7-dihydro-4H-pyrazolo[5,1-c][1,4]oxazine (Intermediate 97) instead of 3-bromo-2-(5-fluoropyridin-2-yl)-6,7-dihydro-4H-pyrazolo[5,1-c][1,4]oxazine (Intermediate 37) and 4-(4,4,5,5-tetramethyl-1,3,2-dioxaborolan-2-yl)-1-((2-(trimethylsilyl)ethoxy)methyl)-1H-pyrazolo[3,4-b]pyridine (Intermediate 23) instead of 4-(4,4,5,5-tetramethyl-1,3,2-dioxaborolan-2-yl)-1-((2-(trimethylsilyl)ethoxy)methyl)-1H-pyrrolo[2,3-b]pyridine (Intermediate 21) in Step A. MS (ESI): mass calcd. for C₁₇H₁₆N₆O₂, 336.1; m/z found, 337.1 [M+H]⁺. ¹H NMR (400 MHz, DMSO-d₆) δ 8.52 (d, J=4.8 Hz, 1H), 8.27 (s, 1H), 7.59 (s, 1H), 7.18-7.15 (m, 2H), 4.86 (s, 2H), 4.12 (s, 2H), 1.37 (s, 6H).

### Example 127: 6,6-Dimethyl-2-(1-methyl-1H-imidazol-4-yl)-3-(1H-pyrazolo[3,4-b]pyridin-4-yl)-6,7-dihydro-4H-pyrazolo[5,1-c][1,4]oxazine

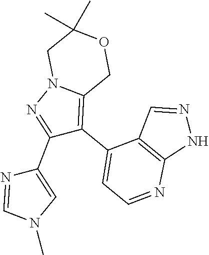 [SMILES: Cn1cnc(-c2nn3c(c2-c2ccnc4[nH]ncc24)COC(C)(C)C3)c1]

The title compound was prepared in a manner analogous to Example 1, except using 3-bromo-6,6-dimethyl-2-(1-methyl-1H-imidazol-4-yl)-6,7-dihydro-4H-pyrazolo[5,1-c][1,4]oxazine (Intermediate 98) instead of 3-bromo-2-(5-fluoropyridin-2-yl)-6,7-dihydro-4H-pyrazolo[5,1-c][1,4]oxazine (Intermediate 37) and 4-(4,4,5,5-tetramethyl-1,3,2-dioxaborolan-2-yl)-1-((2-(trimethylsilyl)ethoxy)methyl)-1H-pyrazolo[3,4-b]pyridine (Intermediate 23) instead of 4-(4,4,5,5-tetramethyl-1,3,2-dioxaborolan-2-yl)-1-((2-(trimethylsilyl)ethoxy)methyl)-1H-pyrrolo[2,3-b]pyridine (Intermediate 21) in Step A. MS (ESI): mass calcd. for C₁₈H₁₉N₇O, 349.2; m/z found, 350.2 [M+H]⁺. ¹H NMR (400 MHz, DMSO-d₆) δ 13.47 (s, 1H), 8.43 (s, 1H), 7.52-7.30 (m, 2H), 7.27-6.97 (m, 2H), 4.81 (s, 2H), 4.03 (s, 2H), 3.60 (s, 3H), 1.42-1.28 (m, 6H).

### Example 128: 2-(5-Fluoropyridin-2-yl)-6,6-dimethyl-3-(pyridin-4-yl)-6,7-dihydro-4H-pyrazolo[5,1-c][1,4]oxazine

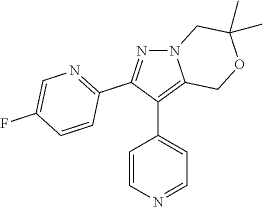 [SMILES: CC1(C)Cn2nc(-c3ccc(F)cn3)c(-c3ccncc3)c2CO1]

The title compound was prepared in a manner analogous to Example 1, Step A, except using 3-bromo-2-(5-fluoropyridin-2-yl)-6,6-dimethyl-6,7-dihydro-4H-pyrazolo[5,1-c][1,4]oxazine (Intermediate 101) instead of 3-bromo-2-(5-fluoropyridin-2-yl)-6,7-dihydro-4H-pyrazolo[5,1-c][1,4]oxazine (Intermediate 37) and pyridin-4-ylboronic acid instead of 4-(4,4,5,5-tetramethyl-1,3,2-dioxaborolan-2-yl)-1-((2-(trimethylsilyl)ethoxy)methyl)-1H-pyrrolo[2,3-b]pyridine (Intermediate 21). MS (ESI): mass calcd. for C₁₈H₁₇FN₄O, 324.1; m/z found, 325.2 [M+H]⁺. ¹H NMR (500 MHz, DMSO-d₆) δ 8.49-8.42 (m, 3H), 7.85-7.74 (m, 2H), 7.21-7.17 (m, 2H), 4.93 (s, 2H), 4.07 (s, 2H), 1.36 (s, 6H).

### Example 129: 3-(2-(Difluoromethyl)pyridin-4-yl)-2-(5-fluoropyridin-2-yl)-6,6-dimethyl-6,7-dihydro-4H-pyrazolo[5,1-c][1,4]oxazine

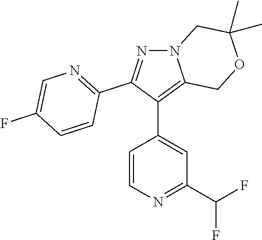 [SMILES: CC1(C)Cn2nc(-c3ccc(F)cn3)c(-c3ccnc(C(F)F)c3)c2CO1]

The title compound was prepared in a manner analogous to Example 1, Step A, except using 3-bromo-2-(5-fluoropyridin-2-yl)-6,6-dimethyl-6,7-dihydro-4H-pyrazolo[5,1-c][1,4]oxazine (Intermediate 101) instead of 3-bromo-2-(5-fluoropyridin-2-yl)-6,7-dihydro-4H-pyrazolo[5,1-c][1,4]oxazine (Intermediate 37) and 2-(difluoromethyl)-4-(4,4,5,5-tetramethyl-1,3,2-dioxaborolan-2-yl)pyridine (Intermediate 28) instead of 4-(4,4,5,5-tetramethyl-1,3,2-dioxaborolan-2-yl)-1-((2-(trimethylsilyl)ethoxy)methyl)-1H-pyrrolo[2,3-b]pyridine (Intermediate 21). MS (ESI): mass calcd. for C₁₉H₁₇F₃N₄O, 374.1; m/z found, 375.2 [M+H]⁺. ¹H NMR (500 MHz, DMSO-d₆) δ 8.57 (d, J=5.13 Hz, 1H), 8.40 (d, J=2.88 Hz, 1H), 7.91-7.85 (m, 1H), 7.84-7.77 (m, 1H), 7.48 (s, 1H), 7.40 (dt, J=5.03, 0.73 Hz, 1H), 6.78 (t, J=56.40 Hz, 1H), 4.96 (s, 2H), 4.08 (s, 2H), 1.36 (s, 6H).

### Example 130: 2-(5-Fluoropyridin-2-yl)-3-(3-methoxypyridin-4-yl)-6,6-dimethyl-6,7-dihydro-4H-pyrazolo[5,1-c][1,4]oxazine

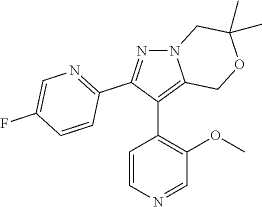 [SMILES: COc1cnccc1-c1c(-c2ccc(F)cn2)nn2c1COC(C)(C)C2]

The title compound was prepared in a manner analogous to Example 1, Step A, except using 3-bromo-2-(5-fluoropyridin-2-yl)-6,6-dimethyl-6,7-dihydro-4H-pyrazolo[5,1-c][1,4]oxazine (Intermediate 101) instead of 3-bromo-2-(5-fluoropyridin-2-yl)-6,7-dihydro-4H-pyrazolo[5,1-c][1,4]oxazine (Intermediate 37) and (3-methoxypyridin-4-yl)boronic acid instead of 4-(4,4,5,5-tetramethyl-1,3,2-dioxaborolan-2-yl)-1-((2-(trimethylsilyl)ethoxy)methyl)-1H-pyrrolo[2,3-b]pyridine (Intermediate 21). MS (ESI): mass calcd. for C₁₉H₁₉FN₄O₂, 354.1; m/z found, 355.2 [M+H]⁺. ¹H NMR (400 MHz, DMSO-d₆) δ 8.34 (dt, J=2.9, 0.7 Hz, 1H), 8.31 (s, 1H), 8.13 (d, J=4.8 Hz, 1H), 7.82-7.70 (m, 2H), 7.17 (dd, J=4.8, 0.5 Hz, 1H), 4.75 (s, 2H), 4.07 (s, 2H), 3.60 (s, 3H), 1.35 (s, 6H).

### Example 131: N-(4-(2-(5-Fluoropyridin-2-yl)-6,6-dimethyl-6,7-dihydro-4H-pyrazolo[5,1-c][1,4]oxazin-3-yl)pyridin-2-yl)propionamide

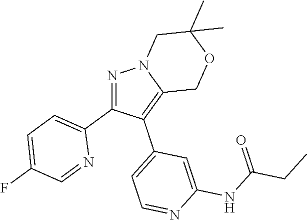 [SMILES: CCC(=O)Nc1cc(-c2c(-c3ccc(F)cn3)nn3c2COC(C)(C)C3)ccn1]

The title compound was prepared in a manner analogous to Example 135, except using propionic acid instead of 2,2-difluorocyclopropanecarboxylic acid. MS (ESI): mass calcd. for C₂₁H₂₂FN₅O₂, 395.2; m/z found, 396.2 [M+H]⁺. ¹H NMR (400 MHz, CD₃OD) δ 8.40 (d, J=2.9 Hz, 1H), 8.18 (dd, J=5.3, 0.8 Hz, 1H), 7.96 (t, J=1.0 Hz, 1H), 7.74 (dd, J=8.7, 4.5 Hz, 1H), 7.71-7.60 (m, 1H), 6.87 (dd, J=5.3, 1.6 Hz, 1H), 5.03 (s, 2H), 4.10 (s, 2H), 2.45 (q, J=7.6 Hz, 2H), 1.45 (s, 6H), 1.20 (t, J=7.6 Hz, 3H).

### Example 132: N-(4-(2-(5-Fluoropyridin-2-yl)-6,6-dimethyl-6,7-dihydro-4H-pyrazolo[5,1-c][1,4]oxazin-3-yl)pyridin-2-yl)isobutyramide

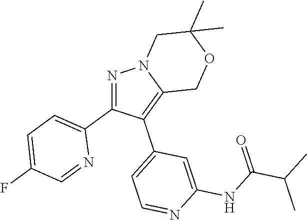 [SMILES: CC(C)C(=O)Nc1cc(-c2c(-c3ccc(F)cn3)nn3c2COC(C)(C)C3)ccn1]

The title compound was prepared in a manner analogous to Example 135, except using isobutyric acid instead of 2,2-difluorocyclopropanecarboxylic acid. MS (ESI): mass calcd. for C₂₂H₂₄FN₅O₂, 409.2; m/z found, 410.2 [M+H]⁺. ¹H NMR (400 MHz, CD₃OD) δ 8.40 (d, J=2.8 Hz, 1H), 8.18 (dd, J=5.3, 0.8 Hz, 1H), 7.97 (t, J=1.1 Hz, 1H), 7.74 (dd, J=8.8, 4.6 Hz, 1H), 7.71-7.62 (m, 1H), 6.87 (dd, J=5.3, 1.6 Hz, 1H), 5.03 (s, 2H), 4.11 (s, 2H), 2.71 (hept, J=6.9 Hz, 1H), 1.46 (s, 6H), 1.21 (d, J=6.9 Hz, 6H).

### Example 133: 3,3,3-Trifluoro-N-(4-(2-(5-fluoropyridin-2-yl)-6,6-dimethyl-6,7-dihydro-4H-pyrazolo[5,1-c][1,4]oxazin-3-yl)pyridin-2-yl)propenamide

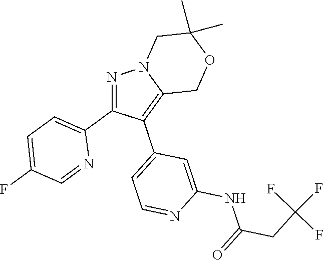 [SMILES: CC1(C)Cn2nc(-c3ccc(F)cn3)c(-c3ccnc(NC(=O)CC(F)(F)F)c3)c2CO1]

The title compound was prepared in a manner analogous to Example 135, except using 3,3,3-trifluoropropionic acid instead of 2,2-difluorocyclopropanecarboxylic acid. MS (ESI): mass calcd. for C₂₁H₁₉F₄N₅O₂, 449.1; m/z found, 450.2 [M+H]⁺. ¹H NMR (400 MHz, CD₃OD) δ 8.40 (d, J=2.9 Hz, 1H), 8.24 (dd, J=5.9, 0.7 Hz, 1H), 7.87 (dd, J=8.8, 4.5 Hz, 1H), 7.79-7.69 (m, 1H), 7.66 (d, J=1.6 Hz, 1H), 7.19 (dd, J=5.9, 1.7 Hz, 1H), 5.06 (s, 2H), 4.13 (s, 2H), 3.53 (q, J=10.4 Hz, 2H), 1.46 (s, 6H).

### Example 134: N-(4-(2-(5-Fluoropyridin-2-yl)-6,6-dimethyl-6,7-dihydro-4H-pyrazolo[5,1-c][1,4]oxazin-3-yl)pyridin-2-yl)cyclopropanecarboxamide

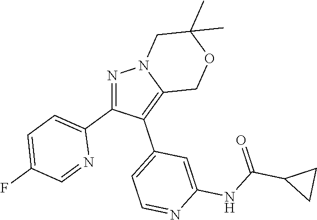 [SMILES: CC1(C)Cn2nc(-c3ccc(F)cn3)c(-c3ccnc(NC(=O)C4CC4)c3)c2CO1]

The title compound was prepared in a manner analogous to Example 135, except using cyclopropanecarboxylic acid instead of 2,2-difluorocyclopropanecarboxylic acid. MS (ESI): mass calcd. for C₂₂H₂₂FN₅O₂, 407.2; m/z found, 408.2 [M+H]⁺. ¹H NMR (400 MHz, CD₃OD) δ 8.40 (d, J=2.8 Hz, 1H), 8.17 (dd, J=5.2, 0.8 Hz, 1H), 7.94 (dd, J=1.6, 0.8 Hz, 1H), 7.73 (dd, J=8.7, 4.5 Hz, 1H), 7.71-7.58 (m, 1H), 6.86 (dd, J=5.3, 1.6 Hz, 1H), 5.51 (s, 1H), 5.01 (s, 2H), 4.10 (s, 2H), 1.88 (tt, J=7.8, 4.7 Hz, 1H), 1.45 (s, 6H), 1.06-0.79 (m, 4H).

### Example 135: 2,2-Difluoro-N-(4-(2-(5-fluoropyridin-2-yl)-6,6-dimethyl-6,7-dihydro-4H-pyrazolo[5,1-c][1,4]oxazin-3-yl)pyridin-2-yl)cyclopropane-1-carboxamide

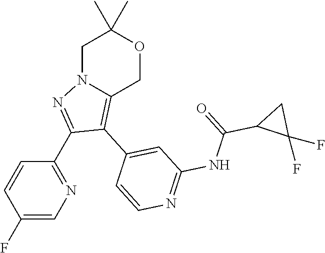 [SMILES: CC1(C)Cn2nc(-c3ccc(F)cn3)c(-c3ccnc(NC(=O)C4CC4(F)F)c3)c2CO1]

To a solution of 4-(2-(5-fluoropyridin-2-yl)-6,6-dimethyl-6,7-dihydro-4H-pyrazolo[5,1-c][1,4]oxazin-3-yl)pyridin-2-amine (Intermediate 104, 29 mg, 0.086 mmol) and 2,2-difluorocyclopropanecarboxylic acid (15.6 mg, 0.128 mmol) in DMF (1 mL) were added Hunig's base (58.9 μL, 0.34 mmol) and HATU (40.6 mg, 0.11 mmol) and the resulting mixture was stirred at room temperature for 18 hours. To it another 2,2-difluorocyclopropanecarboxylic acid (15.6 mg, 0.128 mmol) was added and the resulting mixture was stirred at 50° C. for 18 h. The reaction mixture was purified by reversed phase HPLC method H; to afford 8.1 mg (21%) of the title compound. MS (ESI): mass calcd. for C₂₂H₂₀F₃N₅O₂, 443.2; m/z found, 444.2 [M+H]⁺. ¹H NMR (400 MHz, CD₃OD) δ 8.45-8.38 (m, 1H), 8.22 (dd, J=6.0, 0.6 Hz, 1H), 7.97-7.84 (m, 1H), 7.78-7.64 (m, 1H), 7.54 (dd, J=1.8, 0.6 Hz, 1H), 7.24 (dd, J=6.1, 1.7 Hz, 1H), 5.07 (s, 2H), 4.13 (s, 2H), 2.95-2.73 (m, 1H), 2.29-2.10 (m, 1H), 2.10-1.87 (m, 1H), 1.46 (s, 6H).

### Example 136: N-(4-(2-(5-Fluoropyridin-2-yl)-6,6-dimethyl-6,7-dihydro-4H-pyrazolo[5,1-c][1,4]oxazin-3-yl)pyridin-2-yl)benzamide

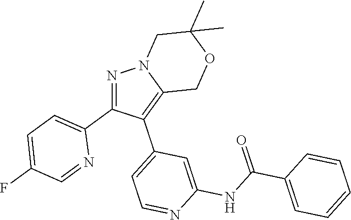 [SMILES: CC1(C)Cn2nc(-c3ccc(F)cn3)c(-c3ccnc(NC(=O)c4ccccc4)c3)c2CO1]

The title compound was prepared in a manner analogous to Example 135, except using benzoic acid instead of 2,2-difluorocyclopropanecarboxylic acid. MS (ESI): mass calcd. for C₂₅H₂₂FN₅O₂, 443.2; m/z found, 444.2 [M+H]⁺. ¹H NMR (400 MHz, CD₃OD) δ 8.43 (d, J=2.8 Hz, 1H), 8.26 (d, J=5.3 Hz, 1H), 8.11 (d, J=1.3 Hz, 1H), 8.02-7.94 (m, 2H), 7.78 (dd, J=8.7, 4.5 Hz, 1H), 7.74-7.58 (m, 2H), 7.60-7.50 (m, 2H), 6.96 (dd, J=5.2, 1.6 Hz, 1H), 5.08 (s, 2H), 4.12 (s, 2H), 1.47 (s, 6H).

### Example 137: 2-(5-Fluoropyridin-2-yl)-6,6-dimethyl-3-(thieno[3,2-b]pyridin-7-yl)-6,7-dihydro-4H-pyrazolo[5,1-c][1,4]oxazine

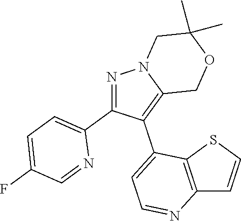 [SMILES: CC1(C)Cn2nc(-c3ccc(F)cn3)c(-c3ccnc4ccsc34)c2CO1]

The title compound was prepared in a manner analogous to Example 1, Step A, except using 3-bromo-2-(5-fluoro-2-pyridyl)-6,6-dimethyl-4,7-dihydropyrazolo[5,1-c][1,4]oxazine (Intermediate 101) instead of 3-bromo-2-(5-fluoropyridin-2-yl)-6,7-dihydro-4H-pyrazolo[5,1-c][1,4]oxazine (Intermediate 37) and 7-(4,4,5,5-tetramethyl-1,3,2-dioxaborolan-2-yl)thieno[3,2-b]pyridine (Intermediate 29) instead of 4-(4,4,5,5-tetramethyl-1,3,2-dioxaborolan-2-yl)-1-((2-(trimethylsilyl)ethoxy)methyl)-1H-pyrrolo[2,3-b]pyridine (Intermediate 21). MS (ESI): mass calcd. for C₂₀H₁₇FN₄OS, 380.1; m/z found, 381.1 [M+H]⁺. ¹H NMR (400 MHz, CD₃OD) δ 8.81 (s, 1H), 8.30 (d, J=5.6 Hz, 1H), 8.00 (s, 2H), 7.70 (dd, J=14.2, 5.6 Hz, 2H), 7.63 (t, J=8.1 Hz, 1H), 4.89 (s, 2H), 4.16 (s, 2H), 1.45 (s, 6H).

### Example 138: 2-(5-Fluoropyridin-2-yl)-6,6-dimethyl-3-(2-methyl-2H-pyrazolo[4,3-b]pyridin-7-yl)-6,7-dihydro-4H-pyrazolo[5,1-c][1,4]oxazine

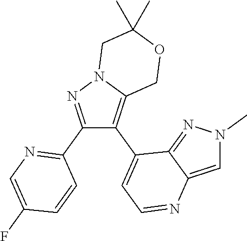 [SMILES: Cn1cc2nccc(-c3c(-c4ccc(F)cn4)nn4c3COC(C)(C)C4)c2n1]

The title compound was prepared in a manner analogous to Example 157, Step A except using 7-bromo-2-methyl-2H-pyrazolo[4,3-b]pyridine (Intermediate 20) instead of 1-benzyl-4-bromo-5-fluoro-6-methyl-1H-pyrazolo[3,4-b]pyridine (Intermediate 25) and microwave irradiation at 120° C. for 2 h instead of conventional heating in Step A. LC-MS (ESI): mass calcd. for C₂₀H₁₉FN₆O 378.16 m/z found 379.2 [M+H]⁺. ¹H NMR (400 MHz, DMSO-d₆) δ 8.59 (s, 1H), 8.37 (d, J=4.3 Hz, 1H), 8.28 (d, J=2.8 Hz, 1H), 7.80-7.69 (m, 2H), 6.98 (d, J=4.5 Hz, 1H), 4.89 (s, 2H), 4.11 (s, 5H), 1.36 (s, 6H).

### Example 139: 2-(5-Fluoropyridin-2-yl)-6,6-dimethyl-3-(1H-pyrazolo[4,3-b]pyridin-7-yl)-6,7-dihydro-4H-pyrazolo[5,1-c][1,4]oxazine

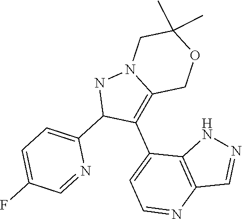

Step A: 2-(5-Fluoropyridin-2-yl)-6,6-dimethyl-3-(1-((2-(trimethylsilyl)ethoxy)methyl)-1H-pyrazolo[4,3-b]pyridin-7-yl)-6,7-dihydro-4H-pyrazolo[5,1-c][1,4]oxazine. The title compound was prepared in a manner analogous to Example 157, Step A except using 7-chloro-1-((2-(trimethylsilyl)ethoxy)methyl)-1H-pyrazolo[4,3-b]pyridine (Intermediate 19) instead of 1-benzyl-4-bromo-5-fluoro-6-methyl-1H-pyrazolo[3,4-b]pyridine (Intermediate 25) and CataCXium® A Pd G3 instead of CataCXium® A-Pd-G2. MS (ESI): mass calcd. for C₂₅H₃₁FN₆O₂Si 494.2 m/z, found 495.3 [M+H]⁺.

Step B: 2-(5-Fluoropyridin-2-yl)-6,6-dimethyl-3-(1H-pyrazolo[4,3-b]pyridin-7-yl)-6,7-dihydro-4H-pyrazolo[5,1-c][1,4]oxazine. 2-(5-Fluoropyridin-2-yl)-6,6-dimethyl-3-(1-((2-(trimethylsilyl)ethoxy)methyl)-1H-pyrazolo[4,3-b]pyridin-7-yl)-6,7-dihydro-4H-pyrazolo[5,1-c][1,4]oxazine (80.0 mg, 0.162 mmol), TFA (1 mL) and dichloromethane (1 mL) were added to a 50 mL round-bottomed. The resultant mixture was stirred at room-temperature for 12 hours. The reaction mixture was concentrated to dryness under reduced pressure. The residue was treated with 2 M NH₃ in MeOH (5 mL) and the resultant mixture stirred at room-temperature for 1 hour. The mixture was concentrated to dryness under reduced pressure. The resulting residue was purified by preparative HPLC Method F: to afford pure product. The product was suspended in water (10 mL), the mixture frozen using dry ice/acetone, and then lyophilized to dryness to afford the title compound (15.5 mg, 26%) as a white solid. LC-MS (ESI): mass calcd. for C₁₉H₁₇FN₆O 364.14 m/z found 365.1 [M+H]⁺. ¹H NMR (400 MHz, CDCl₃): δ 10.61 (br s, 1H), 8.61 (d, J=4.5 Hz, 1H), 8.31 (s, 1H), 8.25 (d, J=2.9 Hz, 1H), 7.66 (dd, J=4.3, 8.7 Hz, 1H), 7.42-7.34 (m, 1H), 7.13 (d, J=4.5 Hz, 1H), 4.85 (s, 2H), 4.10 (s, 2H), 1.46 (s, 6H).

### Example 140: 2-(5-Fluoropyridin-2-yl)-6,6-dimethyl-3-(pyrazolo[1,5-a]pyridin-5-yl)-6,7-dihydro-4H-pyrazolo[5,1-c][1,4]oxazine

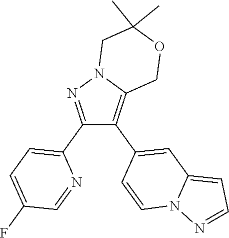 [SMILES: CC1(C)Cn2nc(-c3ccc(F)cn3)c(-c3ccn4nccc4c3)c2CO1]

The title compound was prepared in a manner analogous to Example 1, Step A, except using 3-bromo-2-(5-fluoropyridin-2-yl)-6,6-dimethyl-6,7-dihydro-4H-pyrazolo[5,1-c][1,4]oxazine (Intermediate 101) and 1-pyrazolo[1,5-a]pyridin-5-ylboronic acid instead of 4-(4,4,5,5-tetramethyl-1,3,2-dioxaborolan-2-yl)-1-((2-(trimethylsilyl)ethoxy)methyl)-1H-pyrrolo[2,3-b]pyridine (Intermediate 21). MS (ESI): mass calcd. for C₂₀H₁₈FN₅O, 363.1; m/z found, 364.2 [M+H]⁺.

### Example 141: 3-(1-Ethyl-1H-pyrazolo[3,4-b]pyridin-5-yl)-2-(5-fluoropyridin-2-yl)-6,6-dimethyl-6,7-dihydro-4H-pyrazolo[5,1-c][1,4]oxazine

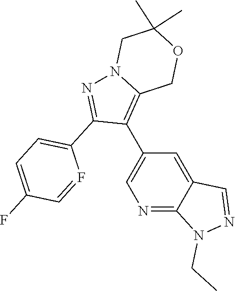

The title compound was prepared in a manner analogous to Example 1, Step A, except using 3-bromo-2-(5-fluoropyridin-2-yl)-6,6-dimethyl-6,7-dihydro-4H-pyrazolo[5,1-c][1,4]oxazine (Intermediate 101) instead of 3-bromo-2-(5-fluoropyridin-2-yl)-6,7-dihydro-4H-pyrazolo[5,1-c][1,4]oxazine (Intermediate 37) and 1-ethyl-5-(4,4,5,5-tetramethyl-1,3,2-dioxaborolan-2-yl)-1H-pyrazolo[3,4-b]pyridine (Intermediate 31) instead of 4-(4,4,5,5-tetramethyl-1,3,2-dioxaborolan-2-yl)-1-((2-(trimethylsilyl)ethoxy)methyl)-1H-pyrrolo[2,3-b]pyridine (Intermediate 21). MS (ESI): mass calcd. for C₂₁H₂₁FN₆O, 392.2; m/z found, 393.2 [M+H]⁺.

### Example 142: 2-(5-Fluoropyridin-2-yl)-6,6-bis(methyl-d₃)-3-(1H-pyrazolo[3,4-b]pyridin-4-yl)-6,7-dihydro-4H-pyrazolo[5,1-c][1,4]oxazine

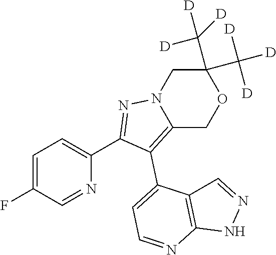 [SMILES: [2H]C([2H])([2H])C1(C([2H])([2H])[2H])Cn2nc(-c3ccc(F)cn3)c(-c3ccnc4[nH]ncc34)c2CO1]

The title compound was prepared in a manner analogous to Example 1, Steps A-B, except using 3-bromo-2-(5-fluoropyridin-2-yl)-6,6-bis(methyl-d₃)-6,7-dihydro-4H-pyrazolo[5,1-c][1,4]oxazine (Intermediate 86) instead of 3-bromo-2-(5-fluoropyridin-2-yl)-6,7-dihydro-4H-pyrazolo[5,1-c][1,4]oxazine (Intermediate 37) and 4-(4,4,5,5-tetramethyl-1,3,2-dioxaborolan-2-yl)-1-((2-(trimethylsilyl)ethoxy)methyl)-1H-pyrazolo[3,4-b]pyridine (Intermediate 23) instead of 4-(4,4,5,5-tetramethyl-1,3,2-dioxaborolan-2-yl)-1-((2-(trimethylsilyl)ethoxy)methyl)-1H-pyrrolo[2,3-b]pyridine (Intermediate 21) and also microwave irradiation at 100° C. for 1 h instead of conventional heating in Step A. MS (ESI): mass calcd. for C₁₉H₁₁D₆FN₆O 370.2; m/z found 371.2 [M+H]⁺. ¹H NMR (400 MHz, DMSO-d₆) δ 13.51 (br s, 1H), 8.45 (d, J=4.6 Hz, 1H), 8.22 (d, J=2.9 Hz, 1H), 7.89-7.85 (m, 1H), 7.80-7.74 (m, 1H), 7.27 (s, 1H), 7.09 (d, J=4.8 Hz, 1H), 4.86 (s, 2H), 4.12 (s, 2H).

### Example 143: 2-(5-Fluoropyridin-2-yl)-6,6-dimethyl-3-(1H-pyrazolo[3,4-b]pyridin-4-yl)-6,7-dihydro-4H-pyrazolo[5,1-c][1,4]oxazine-7,7-d₂

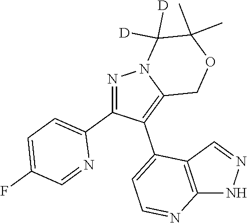 [SMILES: [2H]C1([2H])n2nc(-c3ccc(F)cn3)c(-c3ccnc4[nH]ncc34)c2COC1(C)C]

The title compound was prepared in a manner analogous to Example 1, Steps A-B, except using 3-bromo-2-(5-fluoropyridin-2-yl)-6,6-dimethyl-6,7-dihydro-4H-pyrazolo[5,1-c][1,4]oxazine-7,7-d₂ (Intermediate 144) instead of 3-bromo-2-(5-fluoropyridin-2-yl)-6,7-dihydro-4H-pyrazolo[5,1-c][1,4]oxazine (Intermediate 37) and 4-(4,4,5,5-tetramethyl-1,3,2-dioxaborolan-2-yl)-1-((2-(trimethylsilyl)ethoxy)methyl)-1H-pyrazolo[3,4-b]pyridine (Intermediate 23) instead of 4-(4,4,5,5-tetramethyl-1,3,2-dioxaborolan-2-yl)-1-((2-(trimethylsilyl)ethoxy)methyl)-1H-pyrrolo[2,3-b]pyridine (Intermediate 21) and also microwave irradiation at 100° C. for 1 h instead of conventional heating in Step A. MS (ESI): mass calcd. for C₁₉H₁₅D₂FN₆O 366.2; m/z found 367.1 [M+H]⁺. ¹H NMR (400 MHz, DMSO-d₆) δ 13.51 (br s, 1H), 8.45 (d, J=4.6 Hz, 1H), 8.22 (d, J=2.9 Hz, 1H), 7.91-7.84 (m, 1H), 7.81-7.73 (m, 1H), 7.27 (s, 1H), 7.09 (d, J=4.6 Hz, 1H), 4.87 (s, 2H), 1.37 (s, 6H).

### Example 144: 2-(4-Fluorophenyl)-6,6-dimethyl-3-(1H-pyrazolo[3,4-b]pyridin-4-yl)-6,7-dihydro-4H-pyrazolo[5,1-c][1,4]oxazine

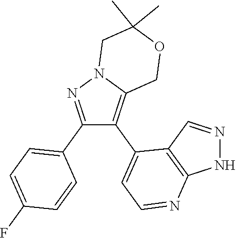 [SMILES: CC1(C)Cn2nc(-c3ccc(F)cc3)c(-c3ccnc4[nH]ncc34)c2CO1]

The title compound was prepared in a manner analogous to Example 1, Steps A-B, except using 3-bromo-2-(4-fluorophenyl)-6,6-dimethyl-6,7-dihydro-4H-pyrazolo[5,1-c][1,4]oxazine (Intermediate 99) instead of 3-bromo-2-(5-fluoropyridin-2-yl)-6,7-dihydro-4H-pyrazolo[5,1-c][1,4]oxazine (Intermediate 37) and (1-((2-(trimethylsilyl)ethoxy)methyl)-1H-pyrazolo[3,4-b]pyridin-4-yl)boronic acid (Intermediate 22) instead of 4-(4,4,5,5-tetramethyl-1,3,2-dioxaborolan-2-yl)-1-((2-(trimethylsilyl)ethoxy)methyl)-1H-pyrrolo[2,3-b]pyridine (Intermediate 21) in step A. MS (ESI): mass calcd. for C₂₀H₁₈FN₅O, 363.2; m/z found, 364.2 [M+H]⁺. ¹H NMR (400 MHz, DMSO-d₆) δ 13.60 (br s, 1H), 8.49 (d, J=4.8 Hz, 1H), 7.41-7.25 (m, 3H), 7.16-6.98 (m, 3H), 4.86 (s, 2H), 4.09 (s, 2H), 1.37 (s, 6H).

### Example 145: 2-(4-Fluorophenyl)-6,6-dimethyl-3-(6-methyl-1H-pyrazolo[3,4-b]pyridin-4-yl)-6,7-dihydro-4H-pyrazolo[5,1-c][1,4]oxazine

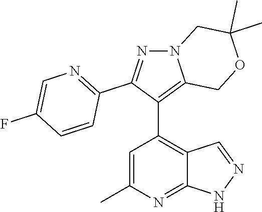 [SMILES: Cc1cc(-c2c(-c3ccc(F)cn3)nn3c2COC(C)(C)C3)c2cn[nH]c2n1]

The title compound was prepared in a manner analogous to Example 1, Steps A-B, except using 3-bromo-2-(4-fluorophenyl)-6,6-dimethyl-6,7-dihydro-4H-pyrazolo[5,1-c][1,4]oxazine (Intermediate 99) instead of 3-bromo-2-(5-fluoropyridin-2-yl)-6,7-dihydro-4H-pyrazolo[5,1-c][1,4]oxazine (Intermediate 37) and 6-methyl-4-(4,4,5,5-tetramethyl-1,3,2-dioxaborolan-2-yl)-1-((2-(trimethylsilyl)ethoxy)methyl)-1H-pyrazolo[3,4-b]pyridine (Intermediate 26) instead of 4-(4,4,5,5-tetramethyl-1,3,2-dioxaborolan-2-yl)-1-((2-(trimethylsilyl)ethoxy)methyl)-1H-pyrrolo[2,3-b]pyridine (Intermediate 21) in Step A. MS (ESI): mass calcd. for C₂₁H₂₀FN₅O, 377.2; m/z found, 378.2 [M+H]⁺. ¹H NMR (400 MHz, DMSO-d₆) δ 13.40 (s, 1H), 7.42-7.36 (m, 2H), 7.26 (s, 1H), 7.18-7.11 (m, 2H), 7.08 (s, 1H), 4.93 (s, 2H), 4.15 (s, 2H), 2.64 (s, 3H), 1.44 (s, 6H).

### Example 146: 2-(5-Chloropyridin-2-yl)-6,6-dimethyl-3-(1H-pyrazolo[3,4-b]pyridin-4-yl)-6,7-dihydro-4H-pyrazolo[5,1-c][1,4]oxazine

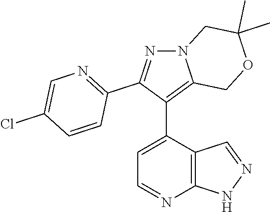 [SMILES: CC1(C)Cn2nc(-c3ccc(Cl)cn3)c(-c3ccnc4[nH]ncc34)c2CO1]

The title compound was prepared in a manner analogous to Example 1, Steps A-B, except using 3-bromo-2-(5-chloropyridin-2-yl)-6,6-dimethyl-6,7-dihydro-4H-pyrazolo[5,1-c][1,4]oxazine (Intermediate 100) instead of 3-bromo-2-(5-fluoropyridin-2-yl)-6,7-dihydro-4H-pyrazolo[5,1-c][1,4]oxazine (Intermediate 37) and 4-(4,4,5,5-tetramethyl-1,3,2-dioxaborolan-2-yl)-1-((2-(trimethylsilyl)ethoxy)methyl)-1H-pyrazolo[3,4-b]pyridine (Intermediate 23) instead of 4-(4,4,5,5-tetramethyl-1,3,2-dioxaborolan-2-yl)-1-((2-(trimethylsilyl)ethoxy)methyl)-1H-pyrrolo[2,3-b]pyridine (Intermediate 21). MS (ESI): mass calcd. for C₁₉H₁₇ClN₆O, 380.1; m/z found, 381.0 [M+H]⁺. ¹H NMR (400 MHz, DMSO-d₆) δ 13.52 (s, 1H), 8.46 (d, J=4.6 Hz, 1H), 8.25 (d, J=2.5 Hz, 1H), 8.10-7.91 (m, 1H), 7.84 (d, J=8.6 Hz, 1H), 7.31 (s, 1H), 7.10 (d, J=4.8 Hz, 1H), 4.86 (s, 2H), 4.13 (s, 2H), 1.38 (s, 6H).

### Example 147: 2-(5-Fluoropyridin-2-yl)-6,6-dimethyl-3-(1H-pyrrolo[2,3-b]pyridin-4-yl)-6,7-dihydro-4H-pyrazolo[5,1-c][1,4]oxazine

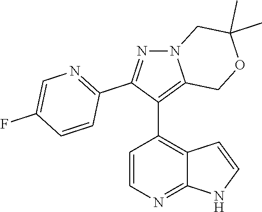 [SMILES: CC1(C)Cn2nc(-c3ccc(F)cn3)c(-c3ccnc4[nH]ccc34)c2CO1]

The title compound was prepared in a manner analogous to Example 1, Step A, except using 3-bromo-2-(5-fluoropyridin-2-yl)-6,6-dimethyl-6,7-dihydro-4H-pyrazolo[5,1-c][1,4]oxazine (Intermediate 101) instead of 3-bromo-2-(5-fluoropyridin-2-yl)-6,7-dihydro-4H-pyrazolo[5,1-c][1,4]oxazine (Intermediate 37) and 4-(4,4,5,5-tetramethyl-1,3,2-dioxaborolan-2-yl)-1-((2-(trimethylsilyl)ethoxy)methyl)-1H-pyrazolo[3,4-b]pyridine (Intermediate 23) instead of 4-(4,4,5,5-tetramethyl-1,3,2-dioxaborolan-2-yl)-1-((2-(trimethylsilyl)ethoxy)methyl)-1H-pyrrolo[2,3-b]pyridine (Intermediate 21). MS (ESI): mass calcd. for C₂₀H₁₈FN₅O, 363.2; m/z found, 364.2 [M+H]⁺. ¹H NMR (500 MHz, DMSO-d₆) δ 11.54 (br s, 1H), 8.27 (d, J=2.75 Hz, 1H), 8.15 (d, J=4.88 Hz, 1H), 7.75-7.65 (m, 2H), 7.30-7.26 (m, 1H), 6.93 (d, J=4.88 Hz, 1H), 5.77 (dd, J=3.38, 1.88 Hz, 1H), 4.76 (s, 2H), 4.11 (s, 2H), 1.37 (s, 6H).

### Example 148: 4-(2-(5-Fluoropyridin-2-yl)-6,6-dimethyl-6,7-dihydro-4H-pyrazolo[5,1-c][1,4]oxazin-3-yl)-1H-pyrrolo[2,3-b]pyridine-3-carbonitrile

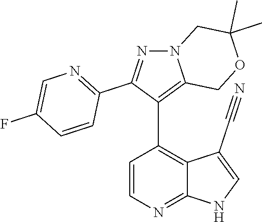 [SMILES: CC1(C)Cn2nc(-c3ccc(F)cn3)c(-c3ccnc4[nH]cc(C#N)c34)c2CO1]

Step A. 4-(2-(5-Fluoropyridin-2-yl)-6,6-dimethyl-6,7-dihydro-4H-pyrazolo[5,1-c][1,4]oxazin-3-yl)-1-((2-(trimethylsilyl)ethoxy)methyl)-1H-pyrrolo[2,3-b]pyridine-3-carbonitrile. 4-((5-Fluoropyridin-2-yl)ethynyl)-1-((2-(trimethylsilyl)ethoxy)methyl)-1H-pyrrolo[2,3-b]pyridine-3-carbonitrile (Intermediate 137, 350 mg, 0.892 mmol), 6,6-dimethyl-6,7-dihydro-4H-[1,2,3]oxadiazolo[4,3-c][1,4]oxazin-8-ium-3-olate (Intermediate 17, 303 mg, 1.78 mmol) and oxydibenzene (4 mL) were added to a 8 mL round-bottomed flask. The mixture was stirred at 200° C. for 3 hours. The reaction mixture was cooled to room-temperature. The mixture was directly purified by FCC (SiO₂, eluent: petroleum ether:ethyl acetate=1:0 to 1:1) to afford the title compound (350 mg, 72%) as a yellow oil. LC-MS (ESI): mass calcd. for C₂₇H₃₁FN₆O₂Si 518.23 m/z found 519.1 [M+H]⁺.

Step B. 4-(2-(5-Fluoropyridin-2-yl)-6,6-dimethyl-6,7-dihydro-4H-pyrazolo[5,1-c][1,4]oxazin-3-yl)-1H-pyrrolo[2,3-b]pyridine-3-carbonitrile. 4-(2-(5-Fluoropyridin-2-yl)-6,6-dimethyl-6,7-dihydro-4H-pyrazolo[5,1-c][1,4]oxazin-3-yl)-1-((2-(trimethylsilyl)ethoxy)methyl)-1H-pyrrolo[2,3-b]pyridine-3-carbonitrile (200 mg, 0.386 mmol), TFA (2 mL), and dichloromethane (6 mL) were added to a 50 mL round-bottomed. The resultant mixture was stirred for 12 hours at room-temperature. The resultant mixture was concentrated to dryness under reduced pressure. The resulting residue was purified by preparative HPLC Method G: to afford pure product. The product was suspended in water (10 mL), the mixture frozen using dry ice/EtOH, and then lyophilized to dryness to afford the title compound (28.1 mg, 19%) as a yellow solid. LC-MS (ESI): mass calcd. for C₂₁H₁₇FN₆O 388.14 m/z found 389.1 [M+H]⁺. ¹H NMR (400 MHz, DMSO-d₆) δ 12.76 (br s, 1H), 8.39-8.24 (m, 2H), 8.14-8.04 (m, 1H), 7.98-7.87 (m, 1H), 7.75-7.65 (m, 1H), 7.11 (d, J=4.6 Hz, 1H), 4.81-4.68 (m, 1H), 4.65-4.52 (m, 1H), 4.20-4.03 (m, 2H), 1.44-1.33 (m, 6H).

### Example 149: 2-(5-Fluoro-2-pyridyl)-6,6-dimethyl-3-(1H-pyrrolo[3,2-b]pyridin-7-yl)-4,7-dihydropyrazolo[5,1-c][1,4]oxazine

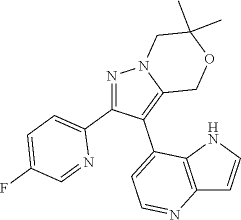 [SMILES: CC1(C)Cn2nc(-c3ccc(F)cn3)c(-c3ccnc4cc[nH]c34)c2CO1]

Step A: 7-((5-Fluoropyridin-2-yl)ethynyl)-1H-pyrrolo[3,2-b]pyridine. 7-bromo-1H-pyrrolo[3,2-b]pyridine (200 mg, 1.02 mmol), 2-ethynyl-5-fluoropyridine (185 mg, 1.53 mmol), CuI (80 mg, 0.42 mmol), Et₃N (4 mL), and DMF (4 mL) were added to a 40 mL sealed tube. The resultant mixture was sparged with N₂ for 5 minutes and then treated PdCl₂(Cy*Phine)₂ (260 mg, 0.203 mmol). The resultant mixture was sparged with N₂ for another 5 minutes and heated at 130° C. for 12 hours. The reaction mixture was gradually cooled to room-temperature, diluted with water (20 mL), and extracted with ethyl acetate (50 mL×3), The combined organic extracts were washed with brine (30 mL×2), dried over anhydrous Na₂SO₄, filtered, and concentrated to dryness under reduced pressure. The resulting residue was purified by FCC (SiO₂, eluent: petroleum ether:ethyl acetate=1:0 to 1:3) to afford the title compound (100 mg, 37%) as a brown solid.

Step B: 2-(5-Fluoropyridin-2-yl)-6,6-dimethyl-3-(1H-pyrrolo[3,2-b]pyridin-7-yl)-6,7-dihydro-4H-pyrazolo[5,1-c][1,4]oxazine. 7-((5-Fluoropyridin-2-yl)ethynyl)-1H-pyrrolo[3,2-b]pyridine (100 mg, 0.422 mmol), 6,6-dimethyl-6,7-dihydro-4H-[1,2,3]oxadiazolo[4,3-c][1,4]oxazin-8-ium-3-olate (Intermediate 17, 86 mg, 0.505 mmol) and toluene (1.5 mL) were added to a 10 mL pressure vial. The resultant mixture was heated at 150° C. via microwave irradiation for 1.5 hours. The reaction mixture was cooled to room-temperature. The resultant mixture was concentrated to dryness under reduced pressure to afford the title compound, which was purified by preparative HPLC Method F: to afford pure product. (20.5 mg, 13%) as a yellow solid. LC-MS (ESI): mass calcd. for C₂₀H₁₈FN₅O 363.15 m/z found 364.1 [M+H]⁺. ¹H NMR (400 MHz, CDCl₃): δ 8.76 (br s, 1H), 8.49 (d, J=4.5 Hz, 1H), 8.32 (d, J=2.8 Hz, 1H), 7.36-7.28 (m, 2H), 7.25-7.19 (m, 1H), 6.97 (d, J=4.8 Hz, 1H), 6.79-6.68 (m, 1H), 4.80 (s, 2H), 4.07 (s, 2H), 1.43 (s, 6H).

### Example 150: 2-(5-Fluoropyridin-2-yl)-6,6-dimethyl-3-(1H-pyrazolo[3,4-b]pyridin-4-yl)-6,7-dihydro-4H-pyrazolo[5,1-c][1,4]oxazine

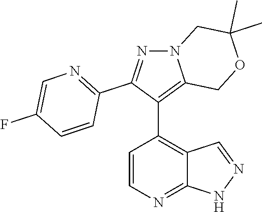 [SMILES: CC1(C)Cn2nc(-c3ccc(F)cn3)c(-c3ccnc4[nH]ncc34)c2CO1]

The title compound was prepared in a manner analogous to Example 1 except using 3-bromo-2-(5-fluoropyridin-2-yl)-6,6-dimethyl-6,7-dihydro-4H-pyrazolo[5,1-c][1,4]oxazine (Intermediate 101) instead of 3-bromo-2-(5-fluoropyridin-2-yl)-6,7-dihydro-4H-pyrazolo[5,1-c][1,4]oxazine (Intermediate 37) and 4-(4,4,5,5-tetramethyl-1,3,2-dioxaborolan-2-yl)-1-((2-(trimethylsilyl)ethoxy)methyl)-1H-pyrazolo[3,4-b]pyridine (Intermediate 23) instead of 4-(4,4,5,5-tetramethyl-1,3,2-dioxaborolan-2-yl)-1-((2-(trimethylsilyl)ethoxy)methyl)-1H-pyrrolo[2,3-b]pyridine (Intermediate 21). MS (ESI): mass calcd. for C₁₉H₁₇FN₆O, 364.1; m/z found, 365.1 [M+H]⁺. ¹H NMR (400 MHz, DMSO-d₆) δ 13.49 (s, 1H), 8.45 (d, J=4.75 Hz, 1H), 8.22 (d, J=3.00 Hz, 1H), 7.87 (dd, J=8.76, 4.13 Hz, 1H), 7.76 (td, J=8.76, 3.00 Hz, 1H), 7.28 (s, 1H), 7.09 (d, J=4.75 Hz, 1H), 4.86 (s, 2H), 4.13 (s, 2H), 1.38 (s, 6H).

### Example 151: 2-(5-Fluoropyridin-2-yl)-6,6-dimethyl-3-(1H-pyrazolo[3,4-b]pyridin-4-yl)-6,7-dihydro-4H-pyrazolo[5,1-c][1,4]oxazine-4,4-d₂

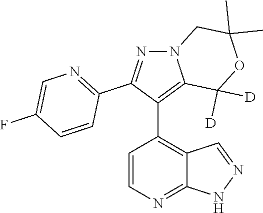 [SMILES: [2H]C1([2H])OC(C)(C)Cn2nc(-c3ccc(F)cn3)c(-c3ccnc4[nH]ncc34)c21]

The title compound was prepared in a manner analogous to Example 1 Step A-B except using 3-bromo-2-(5-fluoropyridin-2-yl)-6,6-dimethyl-6,7-dihydro-4H-pyrazolo[5,1-c][1,4]oxazine-4,4-d₂ (Intermediate 103) instead of 3-bromo-2-(5-fluoropyridin-2-yl)-6,7-dihydro-4H-pyrazolo[5,1-c][1,4]oxazine (Intermediate 37) and (1-((2-(trimethylsilyl)ethoxy)methyl)-1H-pyrazolo[3,4-b]pyridin-4-yl)boronic acid (Intermediate 22) instead of 4-(4,4,5,5-tetramethyl-1,3,2-dioxaborolan-2-yl)-1-((2-(trimethylsilyl)ethoxy)methyl)-1H-pyrrolo[2,3-b]pyridine (Intermediate 21) in Step A and HCl/dioxane instead of TFA/DCM in Step B. MS (ESI): mass calcd. for C₁₉H₁₅D₂FN₆O 366.16 m/z found 367.1 [M+H]⁺. ¹H NMR (400 MHz, DMSO-d₆) δ 13.51 (s, 1H), 8.45 (d, J=4.8 Hz, 1H), 8.22 (d, J=2.6 Hz, 1H), 7.89-7.84 (m, 1H), 7.80-7.73 (m, 1H), 7.27 (s, 1H), 7.09 (d, J=4.8 Hz, 1H), 4.12 (s, 2H), 1.38 (s, 6H).

### Example 152: 3-(3-Bromo-1H-pyrazolo[3,4-b]pyridin-4-yl)-2-(5-fluoropyridin-2-yl)-6,6-dimethyl-6,7-dihydro-4H-pyrazolo[5,1-c][1,4]oxazine

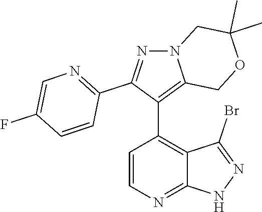 [SMILES: CC1(C)Cn2nc(-c3ccc(F)cn3)c(-c3ccnc4[nH]nc(Br)c34)c2CO1]

NBS (16 mg, 0.091 mmol) was added to a solution consisting of 2-(5-fluoropyridin-2-yl)-6,6-dimethyl-3-(1H-pyrazolo[3,4-b]pyridin-4-yl)-6,7-dihydro-4H-pyrazolo[5,1-c][1,4]oxazine (Example 150, 30 mg, 0.082 mmol), and dichloromethane (5 mL). The resultant mixture was stirred at room-temperature for 2 hours. The reaction mixture was quenched with sat. NaHCO₃ (20 mL) and extracted with dichloromethane (20 mL×2). The combined organic extracts were washed with brine (20 mL), dried over anhydrous Na₂SO₄, filtered, and concentrated to dryness under reduced pressure. The resulting residue was purified by FCC (SiO₂, eluent: petroleum ether:ethyl acetate=3:1 to 0:1) to afford the title compound (35 mg, 96%) as a light yellow oil. ¹H NMR (400 MHz, CDCl₃): δ 12.34 (br s, 1H), 8.68-8.53 (m, 1H), 8.13 (br d, J=2.8 Hz, 1H), 7.59 (dd, J=4.4, 8.8 Hz, 1H), 7.32-7.27 (m, 1H), 7.11-7.06 (m, 1H), 4.77-4.67 (m, 2H), 2.78 (s, 2H), 1.45 (br s, 6H).

### Example 153: 2-(5-Fluoro-2-pyridyl)-6,6-dimethyl-3-(1-methylpyrazolo[3,4-b]pyridin-4-yl)-4,7-dihydropyrazolo[5,1-c][1,4]oxazine

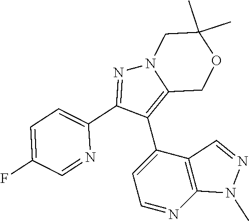 [SMILES: Cn1ncc2c(-c3c(-c4ccc(F)cn4)nn4c3COC(C)(C)C4)ccnc21]

To a solution of 2-(5-fluoropyridin-2-yl)-6,6-dimethyl-3-(1H-pyrazolo[3,4-b]pyridin-4-yl)-6,7-dihydro-4H-pyrazolo[5,1-c][1,4]oxazine (Example 150, 2.0 g, 5.49 mmol) in DMF (16 mL) was added NaH (439.2 mg, 10.98 mmol) at 0° C. and the mixture was stirred for further 30 mins. A solution of CH₃I (1.25 g, 8.80 mmol) in DMF (6 mL) was added at 0° C. The reaction was quenched with H₂O (50 mL), and then extracted with ethyl acetate (100 mL) and the organic phase was evaporated. The residue was slurred in EtOH to afford the title compound (1.0 g, 2.64 mmol, 48%). The mother liquor was reserved for further purification. MS (ESI): mass calcd. for C₂₀H₁₉FN₆O, 378.2; m/z found, 379.2 [M+H]⁺. ¹H NMR (400 MHz, CDCl₃): δ 8.52 (d, J=4.7 Hz, 1H), 8.30 (d, J=2.9 Hz, 1H), 7.44 (dd, J=8.7, 4.4 Hz, 1H), 7.39 (s, 1H), 7.31-7.22 (m, 1H), 6.95 (d, J=4.7 Hz, 1H), 4.84 (s, 2H), 4.13 (s, 3H), 4.12 (s, 2H), 1.43 (s, 6H).

### Example 154: 2-(5-Fluoro-2-pyridyl)-6,6-dimethyl-3-(6-methyl-1H-pyrazolo[3,4-b]pyridin-4-yl)-4,7-dihydropyrazolo[5,1-c][1,4]oxazine

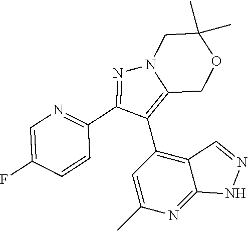 [SMILES: Cc1cc(-c2c(-c3ccc(F)cn3)nn3c2COC(C)(C)C3)c2cn[nH]c2n1]

The title compound was prepared in a manner analogous to Example 1, Steps A-B, except using 6-methyl-4-(4,4,5,5-tetramethyl-1,3,2-dioxaborolan-2-yl)-1-((2-(trimethylsilyl)ethoxy)methyl)-1H-pyrazolo[3,4-b]pyridine (Intermediate 26) instead of 4-(4,4,5,5-tetramethyl-1,3,2-dioxaborolan-2-yl)-1-((2-(trimethylsilyl)ethoxy)methyl)-1H-pyrrolo[2,3-b]pyridine (Intermediate 21) and 3-bromo-2-(5-fluoropyridin-2-yl)-6,6-dimethyl-6,7-dihydro-4H-pyrazolo[5,1-c][1,4]oxazine (Intermediate 101) instead of 3-bromo-2-(5-fluoropyridin-2-yl)-6,7-dihydro-4H-pyrazolo[5,1-c][1,4]oxazine (Intermediate 37) in Step A. MS (ESI): mass calcd. for C₂₀H₁₉FN₆O, 378.2; m/z found, 379.2 [M+H]⁺. ¹H NMR (400 MHz, CDCl₃): δ 11.79 (s, 1H), 8.30 (d, J=2.9 Hz, 1H), 7.49 (dd, J=8.8, 4.4 Hz, 1H), 7.37 (s, 1H), 7.32-7.26 (m, 1H), 6.90 (s, 1H), 4.88 (s, 2H), 4.13 (s, 2H), 2.74 (s, 3H), 1.45 (s, 6H).

### Example 155: 3-(6-Cyclopropyl-1H-pyrazolo[3,4-b]pyridin-4-yl)-2-(5-fluoropyridin-2-yl)-6,6-dimethyl-6,7-dihydro-4H-pyrazolo[5,1-c][1,4]oxazine

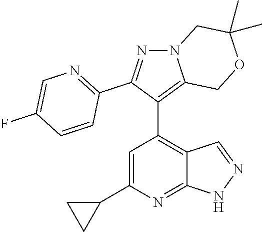 [SMILES: CC1(C)Cn2nc(-c3ccc(F)cn3)c(-c3cc(C4CC4)nc4[nH]ncc34)c2CO1]

Step A. 3-(1-Benzyl-6-cyclopropyl-1H-pyrazolo[3,4-b]pyridin-4-yl)-2-(5-fluoropyridin-2-yl)-6,6-dimethyl-6,7-dihydro-4H-pyrazolo[5,1-c][1,4]oxazine. To a solution of Cs₂CO₃ (41 mg, 0.13 mmol) in H₂O (0.13 mL) was added CataCXium® A Pd G3 (5 mg, 0.0063 mmol), lithium 2-(2-(5-fluoropyridin-2-yl)-6,6-dimethyl-6,7-dihydro-4H-pyrazolo[5,1-c][1,4]oxazin-3-yl)-2-hydroxy-4,4,5,5-tetramethyl-1,3,2-dioxaborolan-2-uide (Intermediate 105, 79 mg, 0.2 mmol), 1-benzyl-4-bromo-6-cyclopropyl-1H-pyrazolo[3,4-b]pyridine (Intermediate 132, 41 mg, 0.13 mmol), and 2-methyl-2-butanol (1 mL). The reaction vial was capped and degassed under vacuum then refilled with N₂. The mixture was heated to 75° C. for 16 h. The reaction mixture was cooled, diluted with H₂O (1 mL), and extracted with EtOAc (3×2 mL). The combined organics were dried (Na₂SO₄) and filtered. Purification by chromatography (silica gel, 0-100% EtOAc/hexanes) afforded 7 mg (11%) of the title compound. MS (ESI): mass calcd. for C₂₉H₂₇FN₆O, 494.2; m/z found, 495.3 [M+H]⁺.

Step B. 3-(6-Cyclopropyl-1H-pyrazolo[3,4-b]pyridin-4-yl)-2-(5-fluoropyridin-2-yl)-6,6-dimethyl-6,7-dihydro-4H-pyrazolo[5,1-c][1,4]oxazine. A solution of 3-(1-benzyl-6-cyclopropyl-1H-pyrazolo[3,4-b]pyridin-4-yl)-2-(5-fluoropyridin-2-yl)-6,6-dimethyl-6,7-dihydro-4H-pyrazolo[5,1-c][1,4]oxazine (17 mg, 0.035 mmol) in neat H₂SO₄ (0.093 mL, 1.7 mmol) was heated to 70° C. for 4 h. The reaction mixture was cooled then quenched with aqueous NaOH and extracted with 4:1 CH₂Cl₂:IPA. The combined organics were dried (Na₂SO₄) and filtered. Purification by chromatography (silica gel, 1% methanol saturated with ammonia/9% methanol/CH₂Cl₂) afforded 6 mg (45%) of the title compound. MS (ESI): mass calcd. for C₂₂H₂₁FN₆O, 404.2; m/z found, 405.2 [M+H]⁺. ¹H NMR (500 MHz, DMSO-d₆) δ 13.20 (s, 1H), 8.24 (d, J=2.88 Hz, 1H), 7.84 (dd, J=8.82, 4.57 Hz, 1H), 7.76 (td, J=8.72, 2.94 Hz, 1H), 7.09 (s, 1H), 7.04 (s, 1H), 4.89 (s, 2H), 4.12 (s, 2H), 2.28-2.17 (m, 1H), 1.38 (s, 6H), 1.06-0.98 (m, 4H).

### Example 156: 3-(3,6-Dimethyl-1H-pyrazolo[3,4-b]pyridin-4-yl)-2-(5-fluoro-2-pyridyl)-6,6-dimethyl-4,7-dihydropyrazolo[5,1-c][1,4]oxazine

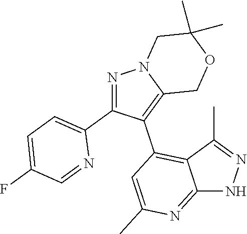 [SMILES: Cc1cc(-c2c(-c3ccc(F)cn3)nn3c2COC(C)(C)C3)c2c(C)n[nH]c2n1]

The title compound was made analogous to Example 155, Step A-B except using 3-bromo-2-(5-fluoropyridin-2-yl)-6,6-dimethyl-6,7-dihydro-4H-pyrazolo[5,1-c][1,4]oxazine (Intermediate 101) instead of 1-benzyl-4-bromo-6-cyclopropyl-1H-pyrazolo[3,4-b]pyridine (Intermediate 132) and 1-Benzyl-3,6-dimethyl-4-(4,4,5,5-tetramethyl-1,3,2-dioxaborolan-2-yl)-1H-pyrazolo[3,4-b]pyridine (Intermediate 24) instead of lithium 2-(2-(5-fluoropyridin-2-yl)-6,6-dimethyl-6,7-dihydro-4H-pyrazolo[5,1-c][1,4]oxazin-3-yl)-2-hydroxy-4,4,5,5-tetramethyl-1,3,2-dioxaborolan-2-uide (Intermediate 105) in Step A. LC-MS (ESI): mass calcd. for C₂₁H₂₁FN₆O 392.18 m/z found 393.1 [M+H]⁺. H NMR (400 MHz, DMSO-d₆) δ 12.92 (s, 1H), 8.14 (d, J=2.7 Hz, 1H), 7.86 (dd, J=4.5, 8.8 Hz, 1H), 7.69 (m, 1H), 6.90 (s, 1H), 4.85 (d, J=15.9 Hz, 1H), 4.49 (d, J=15.9 Hz, 1H), 4.12 (s, 2H), 2.55 (s, 3H), 1.78 (s, 3H), 1.38 (s, 3H), 1.32 (s, 3H).

### Example 157: 3-(5-Fluoro-6-methyl-1H-pyrazolo[3,4-b]pyridin-4-yl)-2-(5-fluoro-2-pyridyl)-6,6-dimethyl-4,7-dihydropyrazolo[5,1-c][1,4]oxazine

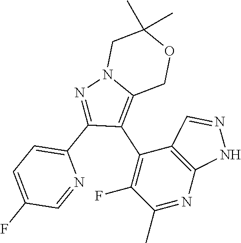 [SMILES: Cc1nc2[nH]ncc2c(-c2c(-c3ccc(F)cn3)nn3c2COC(C)(C)C3)c1F]

Step A: 3-(1-Benzyl-5-fluoro-6-methyl-1H-pyrazolo[3,4-b]pyridin-4-yl)-2-(5-fluoropyridin-2-yl)-6,6-dimethyl-6,7-dihydro-4H-pyrazolo[5,1-c][1,4]oxazine. 1-Benzyl-4-bromo-5-fluoro-6-methyl-1H-pyrazolo[3,4-b]pyridine (Intermediate 25, 100 mg, 0.312 mmol), 3-bromo-2-(5-fluoropyridin-2-yl)-6,6-dimethyl-6,7-dihydro-4H-pyrazolo[5,1-c][1,4]oxazine (Intermediate 101, 102 mg, 0.313 mmol), 4,4,4′,4′,5,5,5′,5′-octamethyl-2,2′-bi(1,3,2-dioxaborolane) (119 mg, 0.469 mmol), Cs₂CO₃ (305 mg, 0.936 mmol), and 1,4-dioxane: H₂O (10:1, 3 mL) were added to a 8 mL sealed tube. The mixture was sparged with N₂ for 5 minutes and then treated with CataCXium® A-Pd-G2 (21 mg, 0.031 mmol). The resultant mixture was heated at 90° C. for 16 hours. The mixture was combined with additional batches and filtered through a pad of Celite® and the pad washed with ethyl acetate (5 mL×2). The filtrate was poured into H₂O (10 mL) and the resultant mixture extracted with ethyl acetate (5 mL×3). The combined organic extracts were washed with brine (10 mL), dried over anhydrous Na₂SO₄, filtered, and concentrated to dryness under reduced pressure. The resulting residue was purified by FCC (SiO₂, eluent: petroleum ether:ethyl acetate=1:0 to 1:1) to afford the pure compound (123 mg) as brown oil and the crude compound, which was further purified by preparative HPLC Method G: to afford pure product (6 mg) as a brown oil. LCMS (ESI): mass calcd. for C₂₇H₂₄F₂N₆O 486.52 m/z, found 487.5 [M+H]⁺. ¹H NMR (400 MHz, DMSO-d₆) δ 8.19 (d, J=2.9 Hz, 1H), 7.99-7.93 (m, 1H), 7.79-7.72 (m, 1H), 7.54 (s, 1H), 7.35-7.17 (m, 5H), 5.68-5.53 (m, 2H), 4.81-4.64 (m, 2H), 4.21-4.08 (m, 2H), 2.57 (d, J=3.5 Hz, 3H), 1.37 (br. d, J=11.9 Hz, 6H)

Step B: 3-(5-Fluoro-6-methyl-1H-pyrazolo[3,4-b]pyridin-4-yl)-2-(5-fluoro-2-pyridyl)-6,6-dimethyl-4,7-dihydropyrazolo[5,1-c][1,4]oxazine. A mixture consisting of 3-(1-benzyl-5-fluoro-6-methyl-1H-pyrazolo[3,4-b]pyridin-4-yl)-2-(5-fluoropyridin-2-yl)-6,6-dimethyl-6,7-dihydro-4H-pyrazolo[5,1-c][1,4]oxazine (109 mg, 0.224 mmol) and H₂SO₄: H₂O (1:1) (2 mL) was stirred at 100° C. for 16 hours. The mixture was combined with additional batches and concentrated to dryness under reduced pressure. The residue was basified with sat·NaHCO₃ to pH=7 to 8. The mixture was poured into water (10 mL) and extracted with dichloromethane (10 mL×3). The combined organic extracts were concentrated to dryness under reduced pressure. The resulting residue was purified by preparative HPLC Method G: to afford pure product. The product was suspended in water (5 mL), the mixture frozen using dry ice/ethanol, and then lyophilized to dryness to afford the title compound (16.5 mg) as a white solid. LCMS (ESI): mass calcd. for C₂₀H₁₈F₂N₆O 396.2 m/z, found 397.0 [M+H]⁺. ¹H NMR (400 MHz, CDCl₃): δ 10.54 (br s, 1H), 8.22 (d, J=2.4 Hz, 1H), 7.63 (dd, J=4.2, 8.6 Hz, 1H), 7.37 (s, 1H), 7.36-7.30 (m, 1H), 5.01-4.82 (m, 1H), 4.75-4.54 (m, 1H), 4.14 (s, 2H), 2.69 (d, J=3.4 Hz, 3H), 1.49-1.44 (m, 6H).

### Example 158: 2-(5-Fluoro-2-pyridyl)-6,6-dimethyl-3-(2-methylpyrazolo[3,4-b]pyridin-4-yl)-4,7-dihydropyrazolo[5,1-c][1,4]oxazine

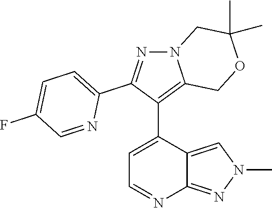 [SMILES: Cn1cc2c(-c3c(-c4ccc(F)cn4)nn4c3COC(C)(C)C4)ccnc2n1]

The title compound was obtained from further purification of the mother liquor reserved from the isolation of 2-(5-fluoro-2-pyridyl)-6,6-dimethyl-3-(1-methylpyrazolo[3,4-b]pyridin-4-yl)-4,7-dihydropyrazolo[5,1-c][1,4]oxazine (Example 153). The filtrate was evaporated and purified by prep HPLC purification to afford the title compound (110 mg, 5.3%) MS (ESI): mass calcd. for C₂₀H₁₉FN₆O, 378.2; m/z found, 379.2 [M+H]⁺. ¹H NMR (400 MHz, CDCl₃): δ 8.67 (d, J=4.4 Hz, 1H), 8.35 (d, J=2.9 Hz, 1H), 7.37-7.27 (m, 2H), 7.21 (td, J=8.4, 2.9 Hz, 1H), 6.89 (d, J=4.4 Hz, 1H), 4.80 (s, 2H), 4.11 (s, 2H), 4.09 (s, 3H), 1.43 (s, 6H).

### Example 159: 2-(5-Fluoropyridin-2-yl)-6,6-dimethyl-3-(1-methyl-1H-pyrazolo[4,3-b]pyridin-7-yl)-6,7-dihydro-4H-pyrazolo[5,1-c][1,4]oxazine

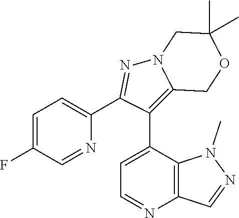 [SMILES: Cn1ncc2nccc(-c3c(-c4ccc(F)cn4)nn4c3COC(C)(C)C4)c21]

A solution of 6,6-dimethyl-6,7-dihydro-4H-[1,2,3]oxadiazolo[4,3-c][1,4]oxazin-8-ium-3-olate (Intermediate 17, 31 mg, 0.18 mmol) and 7-((5-fluoropyridin-2-yl)ethynyl)-1-methyl-1H-pyrazolo[4,3-b]pyridine (Intermediate 136, 64 mg, 0.25 mmol) in xylenes (0.2 mL) was heated to 150° C. for 16 h. The reaction mixture was cooled to room temperature then condensed. Purification by RP HPLC Method H: afforded 11 mg (16%) of the title compound. MS (ESI): mass calcd. for C₂₀H₁₉FN₆O, 378.2; m/z found, 379.0 [M+H]⁺. ¹H NMR (400 MHz, DMSO-d₆) δ 8.46 (d, J=4.38 Hz, 1H), 8.24 (s, 1H), 7.96-8.03 (m, 2H), 7.72 (td, J=8.82, 3.00 Hz, 1H), 7.23 (d, J=4.38 Hz, 1H), 4.83 (d, J=15.88 Hz, 1H), 4.62 (d, J=15.88 Hz, 1H), 4.15 (s, 2H), 3.51 (s, 3H), 1.39 (s, 3H), 1.33 (s, 3H).

### Example 160: 2-(5-Fluoropyridin-2-yl)-6,6-dimethyl-3-(3-methyl-1H-pyrazolo[3,4-b]pyridin-4-yl)-6,7-dihydro-4H-pyrazolo[5,1-c][1,4]oxazine

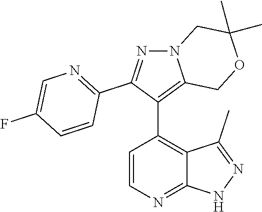 [SMILES: Cc1n[nH]c2nccc(-c3c(-c4ccc(F)cn4)nn4c3COC(C)(C)C4)c12]

3-(3-Bromo-1H-pyrazolo[3,4-b]pyridin-4-yl)-2-(5-fluoropyridin-2-yl)-6,6-dimethyl-6,7-dihydro-4H-pyrazolo[5,1-c][1,4]oxazine (Example 152, 35 mg, 0.079 mmol), 2,4,6-trimethyl-1,3,5,2,4,6-trioxatriborinane (0.316 mL, 0.158 mmol, 0.5 M in THF), and K₂CO₃ (32.7 mg, 0.237 mmol) were dissolved 1,4-dioxane (2 mL) and H₂O (0.2 mL). The resultant mixture was sparged with N₂ for 5 minutes and then treated with Pd(dppf)Cl₂ CH₂Cl₂ (6 mg, 0.008 mmol). The mixture was sparged with N₂ for another 5 minutes and heated at 120° C. for 12 hours. The mixture was concentrated to dryness under reduced pressure. The resulting residue was purified by FCC (SiO₂, eluent: dichloromethane:methanol=1:0 to 10:1) to afford still-impure product (30 mg, crude). The post chromatographic product was further purified by preparative HPLC Method G: to afford pure product. The product was suspended in water (10 mL), the mixture frozen using dry ice/ethanol, and then lyophilized to dryness to afford the title compound (12.5 mg, 42%) as a white solid. MS (ESI): mass calcd. for C₂₀H₁₉FN₆O 378.16 m/z, found 379.0 [M+H]⁺. ¹H NMR (400 MHz, CDCl₃): δ 10.98 (br s, 1H), 8.55 (d, J=4.8 Hz, 1H), 8.23 (d, J=2.8 Hz, 1H), 7.42 (dd, J=4.4, 8.8 Hz, 1H), 7.22 (dt, J=2.8, 8.4 Hz, 1H), 6.98 (d, J=4.8 Hz, 1H), 4.83-4.56 (m, 2H), 4.14 (s, 2H), 2.06 (s, 3H), 1.45 (s, 3H), 1.42 (s, 3H).

### Example 161: 2-(6-Methoxypyridin-2-yl)-6,6-dimethyl-3-(1H-pyrazolo[3,4-b]pyridin-4-yl)-6,7-dihydro-4H-pyrazolo[5,1-c][1,4]oxazine

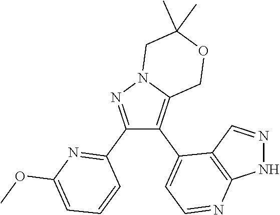 [SMILES: COc1cccc(-c2nn3c(c2-c2ccnc4[nH]ncc24)COC(C)(C)C3)n1]

The title compound was prepared in a manner analogous to Example 1, Steps A-B, except using 3-bromo-2-(6-methoxypyridin-2-yl)-6,6-dimethyl-6,7-dihydro-4H-pyrazolo[5,1-c][1,4]oxazine (Intermediate 106) instead of 3-bromo-2-(5-fluoropyridin-2-yl)-6,7-dihydro-4H-pyrazolo[5,1-c][1,4]oxazine (Intermediate 37) and (1-((2-(trimethylsilyl)ethoxy)methyl)-1H-pyrazolo[3,4-b]pyridin-4-yl)boronic acid (Intermediate 22) instead of 4-(4,4,5,5-tetramethyl-1,3,2-dioxaborolan-2-yl)-1-((2-(trimethylsilyl)ethoxy)methyl)-1H-pyrrolo[2,3-b]pyridine (Intermediate 21) in Step A. MS (ESI): mass calcd. for C₂₀H₂₀N₆O₂ 376.16 m/z found 377.1 [M+H]⁺. ¹H NMR (400 MHz, DMSO-d₆) δ 13.50 (s, 1H), 8.47 (d, J=4.9 Hz, 1H), 7.75-7.68 (m, 1H), 7.54-7.48 (m, 1H), 7.36 (s, 1H), 7.14 (d, J=4.9 Hz, 1H), 6.59 (d, J=8.2 Hz, 1H), 4.83 (s, 2H), 4.12 (s, 2H), 2.66 (s, 3H), 1.37 (s, 5H), 1.40-1.35 (m, 1H).

### Example 162: 2-(3-Chloropyridin-4-yl)-6,6-dimethyl-3-(1H-pyrazolo[3,4-b]pyridin-4-yl)-6,7-dihydro-4H-pyrazolo[5,1-c][1,4]oxazine

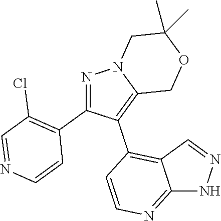 [SMILES: CC1(C)Cn2nc(-c3ccncc3Cl)c(-c3ccnc4[nH]ncc34)c2CO1]

The title compound was prepared in a manner analogous to Example 1, Steps A-B, except using 3-bromo-2-(3-chloropyridin-4-yl)-6,6-dimethyl-6,7-dihydro-4H-pyrazolo[5,1-c][1,4]oxazine (Intermediate 107) instead of 3-bromo-2-(5-fluoropyridin-2-yl)-6,7-dihydro-4H-pyrazolo[5,1-c][1,4]oxazine (Intermediate 37) and (1-((2-(trimethylsilyl)ethoxy)methyl)-1H-pyrazolo[3,4-b]pyridin-4-yl)boronic acid (Intermediate 22) instead of 4-(4,4,5,5-tetramethyl-1,3,2-dioxaborolan-2-yl)-1-((2-(trimethylsilyl)ethoxy)methyl)-1H-pyrrolo[2,3-b]pyridine (Intermediate 21) in Step A. MS (ESI): mass calcd. for C₁₉H₁₇ClN₆O, 380.8; m/z found, 381.1 [M+H]⁺. ¹H NMR (400 MHz, CDCl₃): δ 11.61 (br s, 1H), 8.54-8.46 (m, 3H), 7.57 (s, 1H), 7.42 (d, J=5.0 Hz, 1H), 6.83 (d, J=4.8 Hz, 1H), 4.95 (s, 2H), 4.16 (s, 2H), 1.50 (s, 6H).

### Example 163: 2-(5-Chloro-6-methylpyridin-2-yl)-6,6-dimethyl-3-(1H-pyrazolo[3,4-b]pyridin-4-yl)-6,7-dihydro-4H-pyrazolo[5,1-c][1,4]oxazine

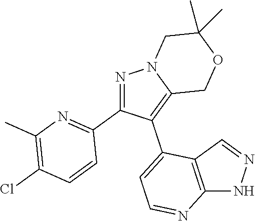 [SMILES: Cc1nc(-c2nn3c(c2-c2ccnc4[nH]ncc24)COC(C)(C)C3)ccc1Cl]

The title compound was prepared in a manner analogous to Example 1, Steps A-B, except using 3-bromo-2-(5-chloro-6-methylpyridin-2-yl)-6,6-dimethyl-6,7-dihydro-4H-pyrazolo[5,1-c][1,4]oxazine (Intermediate 108) instead of 3-bromo-2-(5-fluoropyridin-2-yl)-6,7-dihydro-4H-pyrazolo[5,1-c][1,4]oxazine (Intermediate 37) and (1-((2-(trimethylsilyl)ethoxy)methyl)-1H-pyrazolo[3,4-b]pyridin-4-yl)boronic acid (Intermediate 22) instead of 4-(4,4,5,5-tetramethyl-1,3,2-dioxaborolan-2-yl)-1-((2-(trimethylsilyl)ethoxy)methyl)-1H-pyrrolo[2,3-b]pyridine (Intermediate 21) in Step A. MS (ESI): mass calcd. for C₂₀H₁₉ClN₆O 394.14 m/z found 395.1 [M+H]⁺. ¹H NMR (400 MHz, CDCl₃): δ 11.80 (s, 1H), 8.68-8.56 (m, 1H), 7.65-7.43 (m, 2H), 7.25-7.19 (m, 1H), 7.09-7.01 (m, 1H), 4.98-4.86 (m, 2H), 4.24-4.13 (m, 2H), 2.44-2.33 (m, 3H), 1.66 (s, 6H).

### Example 164: 2-(5-Fluoro-6-methylpyridin-2-yl)-6,6-dimethyl-3-(1H-pyrazolo[3,4-b]pyridin-4-yl)-6,7-dihydro-4H-pyrazolo[5,1-c][1,4]oxazine

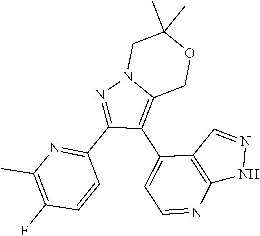 [SMILES: Cc1nc(-c2nn3c(c2-c2ccnc4[nH]ncc24)COC(C)(C)C3)ccc1F]

The title compound was prepared in a manner analogous to Example 1, Steps A-B, except 3-bromo-2-(5-fluoro-6-methylpyridin-2-yl)-6,6-dimethyl-6,7-dihydro-4H-pyrazolo[5,1-c][1,4]oxazine (Intermediate 109) instead of 3-bromo-2-(5-fluoropyridin-2-yl)-6,7-dihydro-4H-pyrazolo[5,1-c][1,4]oxazine (Intermediate 37) and (1-((2-(trimethylsilyl)ethoxy)methyl)-1H-pyrazolo[3,4-b]pyridin-4-yl)boronic acid (Intermediate 22) instead of 4-(4,4,5,5-tetramethyl-1,3,2-dioxaborolan-2-yl)-1-((2-(trimethylsilyl)ethoxy)methyl)-1H-pyrrolo[2,3-b]pyridine (Intermediate 21) in Step A. MS (ESI): mass calcd. for C₂₀H₁₉FN₆O 378.16 m/z found 379.2 [M+H]⁺. ¹H NMR (400 MHz, CDCl₃): δ 11.94 (s, 1H), 8.59 (d, J=4.8 Hz, 1H), 7.50 (s, 1H), 7.24-7.11 (m, 2H), 7.03 (d, J=4.8 Hz, 1H), 4.89 (s, 2H), 4.14 (s, 2H), 2.33 (d, J=2.9 Hz, 3H), 1.45 (s, 6H).

### Example 165: 2-(3,5-Difluoropyridin-4-yl)-6,6-dimethyl-3-(1H-pyrazolo[3,4-b]pyridin-4-yl)-6,7-dihydro-4H-pyrazolo[5,1-c][1,4]oxazine

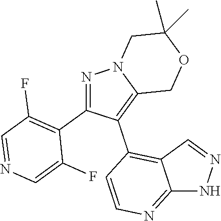 [SMILES: CC1(C)Cn2nc(-c3c(F)cncc3F)c(-c3ccnc4[nH]ncc34)c2CO1]

The title compound was prepared in a manner analogous to Example 1, Steps A-B, except using 3-bromo-2-(3,5-difluoropyridin-4-yl)-6,6-dimethyl-6,7-dihydro-4H-pyrazolo[5,1-c][1,4]oxazine (Intermediate 110) instead of 3-bromo-2-(5-fluoropyridin-2-yl)-6,7-dihydro-4H-pyrazolo[5,1-c][1,4]oxazine (Intermediate 37) and (1-((2-(trimethylsilyl)ethoxy)methyl)-1H-pyrazolo[3,4-b]pyridin-4-yl)boronic acid (Intermediate 22) instead of 4-(4,4,5,5-tetramethyl-1,3,2-dioxaborolan-2-yl)-1-((2-(trimethylsilyl)ethoxy)methyl)-1H-pyrrolo[2,3-b]pyridine (Intermediate 21) in Step A. MS (ESI): mass calcd. for C₁₉H₁₆F₂N₆O, 382.4; m/z found, 383.0 [M+H]⁺. ¹H NMR (400 MHz, DMSO-d₆) δ 13.68 (br s, 1H), 8.52 (s, 2H), 8.45 (d, J=4.8 Hz, 1H), 7.50 (s, 1H), 7.01 (d, J=4.6 Hz, 1H), 4.98 (s, 2H), 4.19 (s, 2H), 1.39 (s, 6H).

### Example 166: 2-(3,5-Difluoropyridin-2-yl)-6,6-dimethyl-3-(1H-pyrazolo[3,4-b]pyridin-4-yl)-6,7-dihydro-4H-pyrazolo[5,1-c][1,4]oxazine

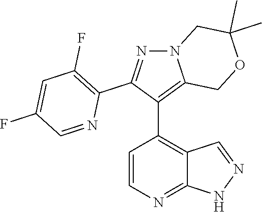 [SMILES: CC1(C)Cn2nc(-c3ncc(F)cc3F)c(-c3ccnc4[nH]ncc34)c2CO1]

The title compound was prepared in a manner analogous to Example 1, Steps A-B, except using 3-bromo-2-(3,5-difluoropyridin-2-yl)-6,6-dimethyl-6,7-dihydro-4H-pyrazolo[5,1-c][1,4]oxazine (Intermediate 111) instead of 3-bromo-2-(5-fluoropyridin-2-yl)-6,7-dihydro-4H-pyrazolo[5,1-c][1,4]oxazine (Intermediate 37) and 4-(4,4,5,5-tetramethyl-1,3,2-dioxaborolan-2-yl)-1-((2-(trimethylsilyl)ethoxy)methyl)-1H-pyrazolo[3,4-b]pyridine (Intermediate 23) instead of 4-(4,4,5,5-tetramethyl-1,3,2-dioxaborolan-2-yl)-1-((2-(trimethylsilyl)ethoxy)methyl)-1H-pyrrolo[2,3-b]pyridine (Intermediate 21) in Step A. MS (ESI): mass calcd. for C₁₉H₁₆F₂N₆O, 382.1; m/z found, 383.2 [M+H]⁺. ¹H NMR (400 MHz, DMSO-d₆) δ 13.56 (s, 1H), 8.44 (d, J=4.63 Hz, 1H), 8.41 (d, J=2.38 Hz, 1H), 7.97-7.91 (m, 1H), 7.26 (d, J=1.25 Hz, 1H), 7.03 (d, J=4.75 Hz, 1H), 4.96 (s, 2H), 4.15 (s, 2H), 1.39 (s, 6H).

### Example 167: 2-(3,5-Difluoropyridin-2-yl)-6,6-dimethyl-3-(6-methyl-1H-pyrazolo[3,4-b]pyridin-4-yl)-6,7-dihydro-4H-pyrazolo[5,1-c][1,4]oxazine

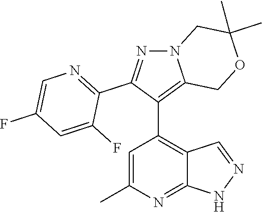 [SMILES: Cc1cc(-c2c(-c3ncc(F)cc3F)nn3c2COC(C)(C)C3)c2cn[nH]c2n1]

The title compound was prepared in a manner analogous to Example 1, Steps A-B, except using 3-bromo-2-(3,5-difluoropyridin-2-yl)-6,6-dimethyl-6,7-dihydro-4H-pyrazolo[5,1-c][1,4]oxazine (Intermediate 111) instead of 3-bromo-2-(5-fluoropyridin-2-yl)-6,7-dihydro-4H-pyrazolo[5,1-c][1,4]oxazine (Intermediate 37) and 6-methyl-4-(4,4,5,5-tetramethyl-1,3,2-dioxaborolan-2-yl)-1-((2-(trimethylsilyl)ethoxy)methyl)-1H-pyrazolo[3,4-b]pyridine (Intermediate 26) instead of 4-(4,4,5,5-tetramethyl-1,3,2-dioxaborolan-2-yl)-1-((2-(trimethylsilyl)ethoxy)methyl)-1H-pyrazolo[3,4-b]pyridine (Intermediate 23) in Step A. MS (ESI): mass calcd. for C₂₀H₁₈F₂N₆O, 396.4; m/z found, 397.2 [M+H]⁺. ¹H NMR (500 MHz, CD₃OD) δ 8.38 (d, J=2.4 Hz, 1H), 7.61-7.54 (m, 1H), 7.28 (s, 1H), 7.01 (s, 1H), 5.05 (s, 2H), 4.20 (s, 2H), 2.67 (s, 3H), 1.50 (s, 6H).

### Example 168: 2-(5-Fluoropyridin-2-yl)-3-(6-methoxy-1,5-naphthyridin-4-yl)-6,6-dimethyl-6,7-dihydro-4H-pyrazolo[5,1-c][1,4]oxazine

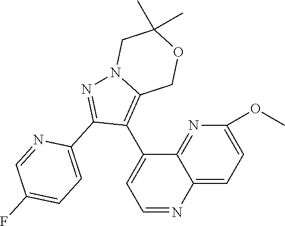 [SMILES: COc1ccc2nccc(-c3c(-c4ccc(F)cn4)nn4c3COC(C)(C)C4)c2n1]

The title compound was prepared in a manner analogous to Example 1, Step A, except using 3-bromo-2-(5-fluoro-2-pyridyl)-6,6-dimethyl-4,7-dihydropyrazolo[5,1-c][1,4]oxazine (Intermediate 101) instead of 3-bromo-2-(5-fluoropyridin-2-yl)-6,7-dihydro-4H-pyrazolo[5,1-c][1,4]oxazine (Intermediate 37) and 2-methoxy-8-(4,4,5,5-tetramethyl-1,3,2-dioxaborolan-2-yl)-1,5-naphthyridine (Intermediate 30) instead of 4-(4,4,5,5-tetramethyl-1,3,2-dioxaborolan-2-yl)-1-((2-(trimethylsilyl)ethoxy)methyl)-1H-pyrrolo[2,3-b]pyridine (Intermediate 21). MS (ESI): mass calcd. for C₂₂H₂₀FN₅O₂, 405.2; m/z found, 406.2 [M+H]⁺. ¹H NMR (400 MHz, CD₃OD) δ 8.66 (d, J=4.6 Hz, 1H), 8.20 (d, J=9.1 Hz, 1H), 8.10 (d, J=2.9 Hz, 1H), 7.67 (dd, J=8.8, 4.5 Hz, 1H), 7.59-7.43 (m, 2H), 7.14 (d, J=9.1 Hz, 1H), 4.88 (s, 2H), 4.14 (s, 2H), 3.67 (s, 3H), 1.45 (s, 6H).

### Example 169: (*R)-6-Ethyl-2-(5-fluoropyridin-2-yl)-6-methyl-3-(1H-pyrazolo[3,4-b]pyridin-4-yl)-6,7-dihydro-4H-pyrazolo[5,1-c][1,4]oxazine

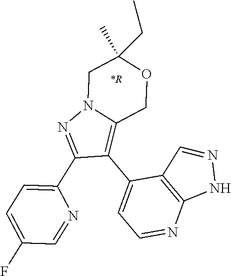 [SMILES: CC[C@]1(C)Cn2nc(-c3ccc(F)cn3)c(-c3ccnc4[nH]ncc34)c2CO1]

The title compound was prepared in a manner analogous to Example 1, Steps A-B, except using 3-bromo-6-ethyl-2-(5-fluoropyridin-2-yl)-6-methyl-6,7-dihydro-4H-pyrazolo[5,1-c][1,4]oxazine (Intermediate 112) instead of 3-bromo-2-(5-fluoropyridin-2-yl)-6,7-dihydro-4H-pyrazolo[5,1-c][1,4]oxazine (Intermediate 37) and 4-(4,4,5,5-tetramethyl-1,3,2-dioxaborolan-2-yl)-1-((2-(trimethylsilyl)ethoxy)methyl)-1H-pyrazolo[3,4-b]pyridine (Intermediate 23) instead of 4-(4,4,5,5-tetramethyl-1,3,2-dioxaborolan-2-yl)-1-((2-(trimethylsilyl)ethoxy)methyl)-1H-pyrrolo[2,3-b]pyridine (Intermediate 21) in Step A. The product of Step B was purified by preparative HPLC Method F: to afford pure product. The product was suspended in water (20 mL), the mixture frozen using dry ice/acetone, and then lyophilized to dryness to afford the title compound. LC-MS (ESI): mass calcd. for C₂₀H₁₉FN₆O 378.16 m/z found 379.2 [M+H]⁺. ¹H NMR (400 MHz, DMSO-d₆) δ 8.45 (d, J=4.6 Hz, 1H), 8.22 (d, J=2.9 Hz, 1H), 7.89-7.85 (m, 1H), 7.80-7.74 (m, 1H), 7.26 (s, 1H), 7.09 (d, J=4.8 Hz, 1H), 4.91-4.79 (m, 2H), 4.15-4.08 (m, 2H), 1.81-1.56 (m, 2H), 1.31 (s, 3H), 0.95 (t, J=7.5 Hz, 3H).

### Example 170: *S-6-Ethyl-2-(5-fluoropyridin-2-(l)-6-methyl-3-(1H-pyrazolo[3,4-b]pyridin-4-yl)-6,7-dihydro-4H-pyrazolo[5,1-c][1,4]oxazine

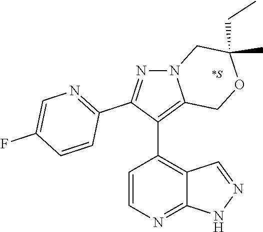 [SMILES: CC[C@@]1(C)Cn2nc(-c3ccc(F)cn3)c(-c3ccnc4[nH]ncc34)c2CO1]

The title compound was prepared in a manner analogous to Example 1, Steps A-B, except using 3-bromo-6-ethyl-2-(5-fluoropyridin-2-yl)-6-methyl-6,7-dihydro-4H-pyrazolo[5,1-c][1,4]oxazine (Intermediate 112) instead of 3-bromo-2-(5-fluoropyridin-2-yl)-6,7-dihydro-4H-pyrazolo[5,1-c][1,4]oxazine (Intermediate 37) and 4-(4,4,5,5-tetramethyl-1,3,2-dioxaborolan-2-yl)-1-((2-(trimethylsilyl)ethoxy)methyl)-1H-pyrazolo[3,4-b]pyridine (Intermediate 23) instead of 4-(4,4,5,5-tetramethyl-1,3,2-dioxaborolan-2-yl)-1-((2-(trimethylsilyl)ethoxy)methyl)-1H-pyrrolo[2,3-b]pyridine (Intermediate 21) in Step A. The product of Step B was purified by preparative HPLC Method F: to afford pure product. The product was suspended in water (20 mL), the mixture frozen using dry ice/acetone, and then lyophilized to dryness to afford the title compound. LC-MS (ESI): mass calcd. for C₂₀H₁₉FN₆O 378.16 m/z found 379.1 [M+H]⁺. ¹H NMR (400 MHz, DMSO-d₆) δ 8.45 (d, J=4.8 Hz, 1H), 8.22 (d, J=3.0 Hz, 1H), 7.89-7.84 (m, 1H), 7.80-7.74 (m, 1H), 7.25 (s, 1H), 7.09 (d, J=4.8 Hz, 1H), 4.91-4.79 (m, 2H), 4.13-4.08 (m, 2H), 1.81-1.60 (m, 2H), 1.31 (s, 3H), 0.95 (t, J=7.5 Hz, 3H).

### Example 171: (*S)-2-(4-Fluorophenyl)-6,6,7-trimethyl-3-(1H-pyrazolo[3,4-b]pyridin-4-yl)-6,7-dihydro-5H-pyrazolo[5,1-b][1,3]oxazine

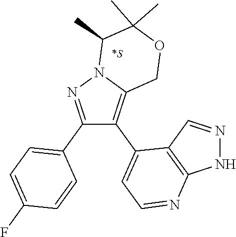 [SMILES: C[C@@H]1n2nc(-c3ccc(F)cc3)c(-c3ccnc4[nH]ncc34)c2COC1(C)C]

The title compound was made in a manner analogous to Example 1, Step A-B, except using 3-bromo-2-(4-fluorophenyl)-6,6,7-trimethyl-6,7-dihydro-4H-pyrazolo[5,1-c][1,4]oxazine (Intermediate 113) instead of 3-bromo-2-(5-fluoropyridin-2-yl)-6,7-dihydro-4H-pyrazolo[5,1-c][1,4]oxazine (Intermediate 37) and 4-(4,4,5,5-tetramethyl-1,3,2-dioxaborolan-2-yl)-1-((2-(trimethylsilyl)ethoxy)methyl)-1H-pyrazolo[3,4-b]pyridine (Intermediate 23) instead of 4-(4,4,5,5-tetramethyl-1,3,2-dioxaborolan-2-yl)-1-((2-(trimethylsilyl)ethoxy)methyl)-1H-pyrrolo[2,3-b]pyridine (Intermediate 21) and also microwave irradiation at 95° C. for 1 hr rather than conventional heating. The product of Step B was purified by SFC Method D. The pure fractions were collected and the volatiles were removed under reduced pressure. The product was suspended in water (10 mL), the mixture frozen using dry ice/EtOH, and then lyophilized to dryness to afford the title compound (118.4 mg, 39%) as a white solid. LC-MS (ESI): mass calcd. for C₂₁H₂₀FN₅O 377.17 m/z found 378.2 [M+H]⁺. ¹H NMR (400 MHz, DMSO-d₆) δ 13.61 (s, 1H), 8.48 (d, J=4.8 Hz, 1H), 7.36-7.27 (m, 3H), 7.14-7.02 (m, 3H), 4.97-4.85 (m, 1H), 4.81-4.69 (m, 1H), 4.21 (q, J=6.6 Hz, 1H), 1.50 (d, J=6.6 Hz, 3H), 1.35 (s, 3H), 1.29 (s, 3H).

### Example 172: (*R)-2-(4-Fluorophenyl)-6,6,7-trimethyl-3-(1H-pyrazolo[3,4-b]pyridin-4-yl)-6,7-dihydro-5H-pyrazolo[5,1-b][1,3]oxazine

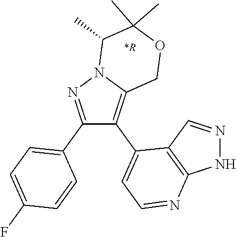 [SMILES: C[C@H]1n2nc(-c3ccc(F)cc3)c(-c3ccnc4[nH]ncc34)c2COC1(C)C]

The title compound was made in a manner analogous to Example 1, Step A-B, except 3-bromo-2-(4-fluorophenyl)-6,6,7-trimethyl-6,7-dihydro-4H-pyrazolo[5,1-c][1,4]oxazine. (Intermediate 113) instead of 3-bromo-2-(5-fluoropyridin-2-yl)-6,7-dihydro-4H-pyrazolo[5,1-c][1,4]oxazine (Intermediate 37) and 4-(4,4,5,5-tetramethyl-1,3,2-dioxaborolan-2-yl)-1-((2-(trimethylsilyl)ethoxy)methyl)-1H-pyrazolo[3,4-b]pyridine (Intermediate 23) instead of 4-(4,4,5,5-tetramethyl-1,3,2-dioxaborolan-2-yl)-1-((2-(trimethylsilyl)ethoxy)methyl)-1H-pyrrolo[2,3-b]pyridine (Intermediate 21) and also microwave irradiation at 95° C. for 1 hr rather than conventional heating. The product of Step B was purified by SFC Method D. The pure fractions were collected and the volatiles were removed under reduced pressure to afford the title compound (120.4 mg, 40%) as a white solid. LC-MS (ESI): mass calcd. for C₂₁H₂₀FN₅O 377.17 m/z found 378.2 [M+H]⁺. ¹H NMR (400 MHz, DMSO-d₆) δ 13.61 (br s, 1H), 8.48 (d, J=4.8 Hz, 1H), 7.36-7.27 (m, 3H), 7.14-7.02 (m, 3H), 4.97-4.85 (m, 1H), 4.81-4.69 (m, 1H), 4.21 (q, J=6.6 Hz, 1H), 1.50 (d, J=6.6 Hz, 3H), 1.35 (s, 3H), 1.29 (s, 3H).

### Example 173: (*R)-2-(5-Fluoropyridin-2-yl)-6,6,7-trimethyl-3-(1H-pyrazolo[3,4-b]pyridin-4-yl)-6,7-dihydro-4H-pyrazolo[5,1-c][1,4]oxazine

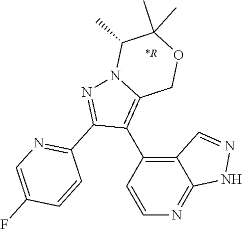 [SMILES: C[C@H]1n2nc(-c3ccc(F)cn3)c(-c3ccnc4[nH]ncc34)c2COC1(C)C]

The title compound was prepared in a manner analogous to Example 1, Steps A-B, except using 3-bromo-2-(5-fluoropyridin-2-yl)-6,6,7-trimethyl-6,7-dihydro-4H-pyrazolo[5,1-c][1,4]oxazine (Intermediate 114) instead of 3-bromo-2-(5-fluoropyridin-2-yl)-6,7-dihydro-4H-pyrazolo[5,1-c][1,4]oxazine (Intermediate 37) and 4-(4,4,5,5-tetramethyl-1,3,2-dioxaborolan-2-yl)-1-((2-(trimethylsilyl)ethoxy)methyl)-1H-pyrazolo[3,4-b]pyridine (Intermediate 23) instead of 4-(4,4,5,5-tetramethyl-1,3,2-dioxaborolan-2-yl)-1-((2-(trimethylsilyl)ethoxy)methyl)-1H-pyrrolo[2,3-b]pyridine (Intermediate 21) in Step A. The product of Step B was further purified by SFC Method C; to afford pure products. The pure fractions were collected and the volatiles were removed under reduced pressure. The product was suspended in water (10 mL), the mixture frozen using dry ice/EtOH, and then lyophilized to dryness to afford the title compound. LC-MS (ESI): mass calcd. for C₂₀H₁₉FN₆O 378.16 m/z found 379.0 [M+H]⁺. ¹H NMR (400 MHz, DMSO-d₆) δ 13.50 (s, 1H), 8.45 (d, J=4.8 Hz, 1H), 8.23 (d, J=3.0 Hz, 1H), 7.92-7.82 (m, 1H), 7.76 (d, J=8.8 Hz, 1H), 7.26 (s, 1H), 7.08 (d, J=4.8 Hz, 1H), 4.99-4.86 (m, 1H), 4.83-4.69 (m, 1H), 4.24 (q, J=6.6 Hz, 1H), 1.51 (d, J=6.7 Hz, 3H), 1.35 (s, 3H), 1.29 (s, 3H).

### Example 174: (*S)-2-(5-Fluoropyridin-2-yl)-6,6,7-trimethyl-3-(1H-pyrazolo[3,4-b]pyridin-4-yl)-6,7-dihydro-4H-pyrazolo[5,1-c][1,4]oxazine

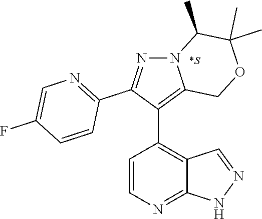 [SMILES: C[C@@H]1n2nc(-c3ccc(F)cn3)c(-c3ccnc4[nH]ncc34)c2COC1(C)C]

The title compound was prepared in a manner analogous to Example 1, Steps A-B, except using 3-bromo-2-(5-fluoropyridin-2-yl)-6,6,7-trimethyl-6,7-dihydro-4H-pyrazolo[5,1-c][1,4]oxazine (Intermediate 114) instead of 3-bromo-2-(5-fluoropyridin-2-yl)-6,7-dihydro-4H-pyrazolo[5,1-c][1,4]oxazine (Intermediate 37) and 4-(4,4,5,5-tetramethyl-1,3,2-dioxaborolan-2-yl)-1-((2-(trimethylsilyl)ethoxy)methyl)-1H-pyrazolo[3,4-b]pyridine (Intermediate 23) instead of 4-(4,4,5,5-tetramethyl-1,3,2-dioxaborolan-2-yl)-1-((2-(trimethylsilyl)ethoxy)methyl)-1H-pyrrolo[2,3-b]pyridine (Intermediate 21) in Step A. The racemic product was further purified by SFC Method C. The pure fractions were collected and the volatiles were removed under reduced pressure. The product was suspended in water (10 mL), the mixture frozen using dry ice/ethanol, and then lyophilized to dryness to afford the title compound (146 mg, 40%) as a white solid. LC-MS (ESI): mass calcd. for C₂₀H₁₉FN₆O 378.16 m/z found 379.0 [M+H]⁺. ¹H NMR (400 MHz, DMSO-d₆) δ 13.50 (s, 1H), 8.45 (d, J=4.8 Hz, 1H), 8.23 (d, J=2.7 Hz, 1H), 7.92-7.83 (m, 1H), 7.76 (d, J=1.0 Hz, 1H), 7.26 (s, 1H), 7.08 (d, J=4.6 Hz, 1H), 4.96-4.87 (m, 1H), 4.84-4.74 (m, 1H), 4.24 (q, J=6.5 Hz, 1H), 1.51 (d, J=6.7 Hz, 3H), 1.36 (s, 3H), 1.30 (s, 3H).

### Example 175: 4-(2-(5-Fluoropyridin-2-yl)-6-methyl-6-(trifluoromethyl)-6,7-dihydro-4H-pyrazolo[5,1-c][1,4]oxazin-3-yl)-N-methylpicolinamide

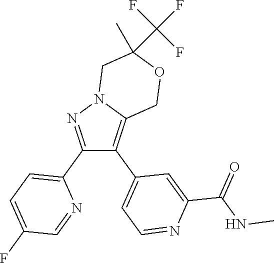 [SMILES: CNC(=O)c1cc(-c2c(-c3ccc(F)cn3)nn3c2COC(C)(C(F)(F)F)C3)ccn1]

The title compound was prepared in a manner analogous to Example 1, Step A-B, except using 3-bromo-2-(5-fluoropyridin-2-yl)-6-methyl-6-(trifluoromethyl)-6,7-dihydro-4H-pyrazolo[5,1-c][1,4]oxazine (Intermediate 115) instead of 3-bromo-2-(5-fluoropyridin-2-yl)-6,7-dihydro-4H-pyrazolo[5,1-c][1,4]oxazine (Intermediate 37), and N-methyl-4-(4,4,5,5-tetramethyl-1,3,2-dioxaborolan-2-yl)picolinamide instead of 4-(4,4,5,5-tetramethyl-1,3,2-dioxaborolan-2-yl)-1-((2-(trimethylsilyl)ethoxy)methyl)-1H-pyrrolo[2,3-b]pyridine (Intermediate 21). MS (ESI): mass calcd. for C₂₀H₁₇F₄N₅O₂, 435.1; m/z found, 436.1 [M+H]⁺. ¹H NMR (600 MHz, DMSO-d₆) δ 8.73 (q, J=4.8 Hz, 1H), 8.54 (dd, J=5.0, 0.8 Hz, 1H), 8.41 (d, J=3.0 Hz, 1H), 7.89-7.86 (m, 1H), 7.82 (td, J=8.8, 2.9 Hz, 1H), 7.77 (dd, J=1.8, 0.8 Hz, 1H), 7.44 (dd, J=5.0, 1.8 Hz, 1H), 5.23-5.05 (m, 2H), 4.49 (s, 2H), 2.80 (d, J=4.9 Hz, 3H), 1.59 (s, 3H).

### Example 176: 2-(5-Fluoropyridin-2-yl)-6-methyl-3-(1H-pyrazolo[3,4-b]pyridin-4-yl)-6-(trifluoromethyl)-6,7-dihydro-4H-pyrazolo[5,1-c][1,4]oxazine

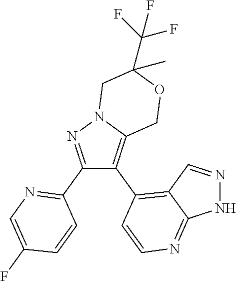 [SMILES: CC1(C(F)(F)F)Cn2nc(-c3ccc(F)cn3)c(-c3ccnc4[nH]ncc34)c2CO1]

The title compound was prepared in a manner analogous to Example 1, Step A-B, except using 3-bromo-2-(5-fluoropyridin-2-yl)-6-methyl-6-(trifluoromethyl)-6,7-dihydro-4H-pyrazolo[5,1-c][1,4]oxazine (Intermediate 115) instead of 3-bromo-2-(5-fluoropyridin-2-yl)-6,7-dihydro-4H-pyrazolo[5,1-c][1,4]oxazine (Intermediate 37), and 4-(4,4,5,5-tetramethyl-1,3,2-dioxaborolan-2-yl)-1-((2-(trimethylsilyl)ethoxy)methyl)-1H-pyrazolo[3,4-b]pyridine (Intermediate 23) instead of 4-(4,4,5,5-tetramethyl-1,3,2-dioxaborolan-2-yl)-1-((2-(trimethylsilyl)ethoxy)methyl)-1H-pyrrolo[2,3-b]pyridine (Intermediate 21). MS (ESI): mass calcd. for C₁₉H₁₄F₄N₆O, 418.1; m/z found, 419.1 [M+H]⁺. ¹H NMR (600 MHz, DMSO-d₆) δ 13.53 (s, 1H), 8.47 (d, J=4.7 Hz, 1H), 8.24 (d, J=3.0 Hz, 1H), 7.88 (dd, J=8.8, 4.5 Hz, 1H), 7.79 (td, J=8.7, 3.0 Hz, 1H), 7.30 (d, J=1.4 Hz, 1H), 7.09 (d, J=4.7 Hz, 1H), 5.15-4.99 (m, 2H), 4.53 (s, 2H), 1.60 (s, 3H).

### Example 177: (*S)-2-(5-Fluoropyridin-2-yl)-6-methyl-3-(1H-pyrazolo[3,4-b]pyridin-4-yl)-6-(trifluoromethyl)-6,7-dihydro-4H-pyrazolo[5,1-c][1,4]oxazine

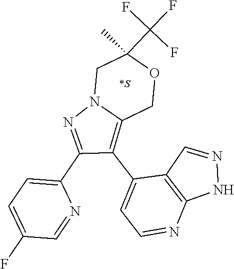 [SMILES: C[C@@]1(C(F)(F)F)Cn2nc(-c3ccc(F)cn3)c(-c3ccnc4[nH]ncc34)c2CO1]

The title compound was prepared in a manner analogous to Example 1, Step A-B, except using 3-bromo-2-(5-fluoropyridin-2-yl)-6-methyl-6-(trifluoromethyl)-6,7-dihydro-4H-pyrazolo[5,1-c][1,4]oxazine (Intermediate 115) instead of 3-bromo-2-(5-fluoropyridin-2-yl)-6,7-dihydro-4H-pyrazolo[5,1-c][1,4]oxazine (Intermediate 37), and 4-(4,4,5,5-tetramethyl-1,3,2-dioxaborolan-2-yl)-1-((2-(trimethylsilyl)ethoxy)methyl)-1H-pyrazolo[3,4-b]pyridine (Intermediate 23) instead of 4-(4,4,5,5-tetramethyl-1,3,2-dioxaborolan-2-yl)-1-((2-(trimethylsilyl)ethoxy)methyl)-1H-pyrrolo[2,3-b]pyridine (Intermediate 21). The product of Step B was further purified by SFC over Method I. The pure fractions were collected and the solvent was removed under reduced pressure to afford the title compound. MS (ESI): mass calcd. for C₁₉H₁₄F₄N₆O, 418.1; m/z found, 419.1 [M+H]⁺. ¹H NMR (600 MHz, DMSO-d₆) δ 13.54 (s, 1H), 8.47 (d, J=4.7 Hz, 1H), 8.24 (d, J=3.0 Hz, 1H), 7.88 (m, 1H), 7.79 (td, J=8.7, 2.9 Hz, 1H), 7.30 (s, 1H), 7.09 (d, J=4.7 Hz, 1H), 5.18-4.91 (m, 2H), 4.53 (s, 2H), 1.60 (s, 3H).

### Example 178: (*R)-2-(5-Fluoropyridin-2-yl)-6-methyl-3-(1H-pyrazolo[3,4-b]pyridin-4-yl)-6-(trifluoromethyl)-6,7-dihydro-4H-pyrazolo[5,1-c][1,4]oxazine

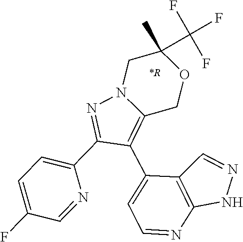 [SMILES: C[C@]1(C(F)(F)F)Cn2nc(-c3ccc(F)cn3)c(-c3ccnc4[nH]ncc34)c2CO1]

The title compound was prepared in a manner analogous to Example 1, Step A-B, except using 3-bromo-2-(5-fluoropyridin-2-yl)-6-methyl-6-(trifluoromethyl)-6,7-dihydro-4H-pyrazolo[5,1-c][1,4]oxazine (Intermediate 115) instead of 3-bromo-2-(5-fluoropyridin-2-yl)-6,7-dihydro-4H-pyrazolo[5,1-c][1,4]oxazine (Intermediate 37), and 4-(4,4,5,5-tetramethyl-1,3,2-dioxaborolan-2-yl)-1-((2-(trimethylsilyl)ethoxy)methyl)-1H-pyrazolo[3,4-b]pyridine (Intermediate 23) instead of 4-(4,4,5,5-tetramethyl-1,3,2-dioxaborolan-2-yl)-1-((2-(trimethylsilyl)ethoxy)methyl)-1H-pyrrolo[2,3-b]pyridine (Intermediate 21). The product of Step B was further purified by SFC Method I. The pure fractions were collected and the solvent was removed under reduced pressure to afford the title compound. MS (ESI): mass calcd. for C₁₉H₁₄F₄N₆O, 418.1; m/z found, 419.1 [M+H]⁺. ¹H NMR (600 MHz, DMSO-d₆) δ 13.53 (s, 1H), 8.46 (dd, J=4.7, 0.8 Hz, 1H), 8.24 (d, J=2.9 Hz, 1H), 7.87 (dd, J=8.8, 4.5 Hz, 1H), 7.78 (td, J=8.7, 3.0 Hz, 1H), 7.30 (s, 1H), 7.08 (dd, J=4.8, 1.3 Hz, 1H), 5.20-4.91 (m, 2H), 4.53 (s, 2H), 1.60 (s, 3H).

### Example 179: (*S)-2-(5-Fluoropyridin-2-yl)-6-methyl-3-(6-methyl-1H-pyrazolo[3,4-b]pyridin-4-yl)-6-(trifluoromethyl)-6,7-dihydro-4H-pyrazolo[5,1-c][1,4]oxazine

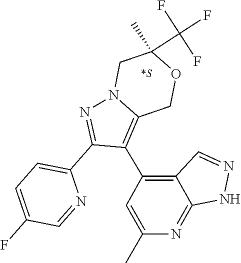 [SMILES: Cc1cc(-c2c(-c3ccc(F)cn3)nn3c2CO[C@](C)(C(F)(F)F)C3)c2cn[nH]c2n1]

The title compound was prepared in a manner analogous to Example 1, Step A-B, except using 3-bromo-2-(5-fluoropyridin-2-yl)-6-methyl-6-(trifluoromethyl)-6,7-dihydro-4H-pyrazolo[5,1-c][1,4]oxazine (Intermediate 115) instead of 3-bromo-2-(5-fluoropyridin-2-yl)-6,7-dihydro-4H-pyrazolo[5,1-c][1,4]oxazine (Intermediate 37), and 6-methyl-4-(4,4,5,5-tetramethyl-1,3,2-dioxaborolan-2-yl)-1-((2-(trimethylsilyl)ethoxy)methyl)-1H-pyrazolo[3,4-b]pyridine (Intermediate 26) instead of 4-(4,4,5,5-tetramethyl-1,3,2-dioxaborolan-2-yl)-1-((2-(trimethylsilyl)ethoxy)methyl)-1H-pyrrolo[2,3-b]pyridine (Intermediate 21). MS (ESI): mass calcd. for C₂₀H₁₆F₄N₆O, 432.1; m/z found, 433.1 [M+H]⁺. ¹H NMR (600 MHz, DMSO-d₆) δ 13.29 (s, 1H), 8.25 (d, J=2.9 Hz, 1H), 7.86 (dd, J=8.8, 4.5 Hz, 1H), 7.78 (td, J=8.7, 3.0 Hz, 1H), 7.14 (d, J=1.4 Hz, 1H), 7.00 (s, 1H), 5.16-4.96 (m, 2H), 4.52 (s, 2H), 2.58 (s, 3H), 1.60 (s, 3H).

### Example 180: (*R)-2-(5-Fluoropyridin-2-yl)-6-methyl-3-(6-methyl-1H-pyrazolo[3,4-b]pyridin-4-yl)-6-(trifluoromethyl)-6,7-dihydro-4H-pyrazolo[5,1-c][1,4]oxazine

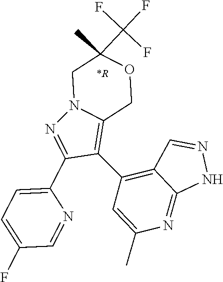 [SMILES: Cc1cc(-c2c(-c3ccc(F)cn3)nn3c2CO[C@@](C)(C(F)(F)F)C3)c2cn[nH]c2n1]

The title compound was prepared in a manner analogous to Example 1, Step A-B, except using 3-bromo-2-(5-fluoropyridin-2-yl)-6-methyl-6-(trifluoromethyl)-6,7-dihydro-4H-pyrazolo[5,1-c][1,4]oxazine (Intermediate 115) instead of 3-bromo-2-(5-fluoropyridin-2-yl)-6,7-dihydro-4H-pyrazolo[5,1-c][1,4]oxazine (Intermediate 37), and 6-methyl-4-(4,4,5,5-tetramethyl-1,3,2-dioxaborolan-2-yl)-1-((2-(trimethylsilyl)ethoxy)methyl)-1H-pyrazolo[3,4-b]pyridine (Intermediate 26) instead of 4-(4,4,5,5-tetramethyl-1,3,2-dioxaborolan-2-yl)-1-((2-(trimethylsilyl)ethoxy)methyl)-1H-pyrrolo[2,3-b]pyridine (Intermediate 21). MS (ESI): mass calcd. for C₂₀H₁₆F₄N₆O, 432.1; m/z found, 433.1 [M+H]⁺. ¹H NMR (600 MHz, DMSO-d₆) δ 13.29 (s, 1H), 8.25 (d, J=2.9 Hz, 1H), 7.88-7.83 (m, 1H), 7.78 (td, J=8.7, 2.9 Hz, 1H), 7.14 (d, J=1.4 Hz, 1H), 7.00 (s, 1H), 5.16-5.00 (m, 2H), 4.55-4.48 (m, 2H), 2.58 (s, 3H), 1.60 (s, 3H).

### Example 181: 2-(4-Fluorophenyl)-3-(1H-pyrazolo[3,4-b]pyridin-4-yl)-6,7-dihydropyrazolo[5,1-c][1,4]oxazin-4-one

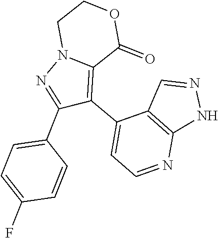 [SMILES: O=C1OCCn2nc(-c3ccc(F)cc3)c(-c3ccnc4[nH]ncc34)c21]

The title compound was prepared in a manner analogous to Example 73, Steps A-B, except using 3-bromo-2-(4-fluorophenyl)-6,7-dihydro-4H-pyrazolo[5,1-c][1,4]oxazin-4-one (Intermediate 116) instead of 3-bromo-2-(4-chloro-3-fluorophenyl)-6,7-dihydro-4H-pyrazolo[5,1-c][1,4]oxazine (Intermediate 67) and 4-(4,4,5,5-tetramethyl-1,3,2-dioxaborolan-2-yl)-1-((2-(trimethylsilyl)ethoxy)methyl)-1H-pyrazolo[3,4-b]pyridine (Intermediate 23) instead of 6-methyl-4-(4,4,5,5-tetramethyl-1,3,2-dioxaborolan-2-yl)-1-((2-(trimethylsilyl)ethoxy)methyl)-1H-pyrazolo[3,4-b]pyridine (Intermediate 26) in Step A. MS (ESI): mass calcd. for C₁₈H₁₂FN₅O₂ 349.1; m/z found 350.0 [M+H]⁺. ¹H NMR (400 MHz, DMSO-d₆) δ 13.66 (s, 1H), 8.52 (m, 1H), 7.71 (s, 1H), 7.33-7.23 (m, 2H), 7.17-7.09 (m, 3H), 4.88 (m, 2H), 4.67 (m, 2H).

### Example 182: 2-(5-Fluoro-2-pyridyl)-3-(1H-pyrazolo[3,4-b]pyridin-4-yl)-6,7-dihydropyrazolo[5,1-c][1,4]oxazin-4-one

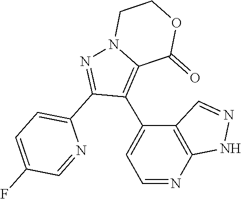 [SMILES: O=C1OCCn2nc(-c3ccc(F)cn3)c(-c3ccnc4[nH]ncc34)c21]

The title compound was prepared in a manner analogous to Example 73, Steps A-B, except using 3-Bromo-2-(5-fluoropyridin-2-yl)-6,7-dihydro-4H-pyrazolo[5,1-c][1,4]oxazin-4-one (Intermediate 117) instead of 3-bromo-2-(4-chloro-3-fluorophenyl)-6,7-dihydro-4H-pyrazolo[5,1-c][1,4]oxazine (Intermediate 67) and 4-(4,4,5,5-tetramethyl-1,3,2-dioxaborolan-2-yl)-1-((2-(trimethylsilyl)ethoxy)methyl)-1H-pyrazolo[3,4-b]pyridine (Intermediate 23) instead of 6-methyl-4-(4,4,5,5-tetramethyl-1,3,2-dioxaborolan-2-yl)-1-((2-(trimethylsilyl)ethoxy)methyl)-1H-pyrazolo[3,4-b]pyridine (Intermediate 26) in Step A. MS (ESI): mass calcd. for C₁₇H₁₁FN₆O₂ 350.3; m/z found 351.1 [M+H]⁺. ¹H NMR (400 MHz, DMSO-d₆) δ 8.49 (m, 1H), 8.26 (m, 1H), 7.87-7.74 (m, 2H), 7.67 (s, 1H), 7.13 (m, 1H), 4.90 (m, 2H), 4.76-4.65 (m, 2H).

### Example 183: N-(4-(2-(5-Fluoropyridin-2-yl)-6,7-dihydro-5H-pyrazolo[5,1-b][1,3]oxazin-3-yl)pyridin-2-yl)acetamide

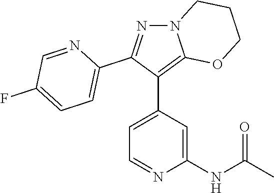 [SMILES: CC(=O)Nc1cc(-c2c(-c3ccc(F)cn3)nn3c2OCCC3)ccn1]

The title compound was prepared in a manner analogous to Example 1, Step A, except using 3-bromo-2-(5-fluoropyridin-2-yl)-6,7-dihydro-5H-pyrazolo[5,1-b][1,3]oxazine (Intermediate 119) instead of 3-bromo-2-(5-fluoropyridin-2-yl)-6,7-dihydro-4H-pyrazolo[5,1-c][1,4]oxazine (Intermediate 37) and N-(4-(4,4,5,5-tetramethyl-1,3,2-dioxaborolan-2-yl)pyridin-2-yl)acetamide instead of 4-(4,4,5,5-tetramethyl-1,3,2-dioxaborolan-2-yl)-1-((2-(trimethylsilyl)ethoxy)methyl)-1H-pyrrolo[2,3-b]pyridine (Intermediate 21) in Step A. MS (ESI): mass calcd. for C₁₈H₁₆FN₅O₂, 353.1; m/z found, 354.0 [M+H]⁺. ¹H NMR (500 MHz, CD₃OD) S 8.42-8.41 (m, 1H), 8.08 (dd, J=5.4, 0.8 Hz, 1H), 7.95 (s, 1H), 7.70-7.64 (m, 2H), 7.02 (dd, J=5.4, 1.6 Hz, 1H), 4.51-4.46 (m, 2H), 4.26 (t, J=6.2 Hz, 2H), 2.43-2.33 (m, 2H), 2.10 (s, 3H).

### Example 184: 2-(4-Fluorophenyl)-3-(1H-pyrazolo[3,4-b]pyridin-4-yl)-6,7-dihydro-5H-pyrazolo[5,1-b][1,3]oxazine

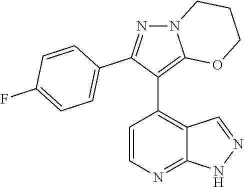 [SMILES: Fc1ccc(-c2nn3c(c2-c2ccnc4[nH]ncc24)OCCC3)cc1]

The title compound was prepared in a manner analogous to Example 1, Step A-B except using 3-bromo-2-(4-fluorophenyl)-6,7-dihydro-5H-pyrazolo[5,1-b][1,3]oxazine (Intermediate 118) instead of 3-bromo-2-(5-fluoropyridin-2-yl)-6,7-dihydro-4H-pyrazolo[5,1-c][1,4]oxazine (Intermediate 37) and 4-(4,4,5,5-tetramethyl-1,3,2-dioxaborolan-2-yl)-1-((2-(trimethylsilyl)ethoxy)methyl)-1H-pyrazolo[3,4-b]pyridine (Intermediate 23) instead of 4-(4,4,5,5-tetramethyl-1,3,2-dioxaborolan-2-yl)-1-((2-(trimethylsilyl)ethoxy)methyl)-1H-pyrrolo[2,3-b]pyridine (Intermediate 21) in Step A. MS (ESI): mass calcd. for C₁₈H₁₄FN₅O, 335.1; m/z found, 336.1 [M+H]⁺. ¹H NMR (400 MHz, CD₃OD) δ 8.38 (d, J=5.0 Hz, 1H), 7.59 (s, 1H), 7.48-7.27 (m, 2H), 7.15-6.96 (m, 3H), 4.59-4.44 (m, 2H), 4.32 (t, J=6.2 Hz, 2H), 3.38 (s, 1H), 2.44 (qd, J=6.1, 4.4 Hz, 2H).

### Example 185: 2-(4-Fluorophenyl)-3-(6-methyl-1H-pyrazolo[3,4-b]pyridin-4-yl)-6,7-dihydro-5H-pyrazolo[5,1-b][1,3]oxazine

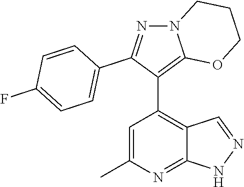 [SMILES: Cc1cc(-c2c(-c3ccc(F)cc3)nn3c2OCCC3)c2cn[nH]c2n1]

The title compound was prepared in a manner analogous to Example 1, Steps A-B, except using 3-bromo-2-(4-fluorophenyl)-6,7-dihydro-5H-pyrazolo[5,1-b][1,3]oxazine (Intermediate 118) instead of 3-bromo-2-(5-fluoropyridin-2-yl)-6,7-dihydro-4H-pyrazolo[5,1-c][1,4]oxazine (Intermediate 37) and 6-methyl-4-(4,4,5,5-tetramethyl-1,3,2-dioxaborolan-2-yl)-1-((2-(trimethylsilyl)ethoxy)methyl)-1H-pyrazolo[3,4-b]pyridine (Intermediate 26) instead of 4-(4,4,5,5-tetramethyl-1,3,2-dioxaborolan-2-yl)-1-((2-(trimethylsilyl)ethoxy)methyl)-1H-pyrrolo[2,3-b]pyridine (Intermediate 21) in Step A. MS (ESI): mass calcd. for C₁₉H₁₆FN₅O, 349.1; m/z found, 350.1 [M+H]⁺. ¹H NMR (500 MHz, CD₃OD) δ 7.39 (s, 1H), 7.38-7.30 (m, 2H), 7.08-6.95 (m, 2H), 6.89 (s, 1H), 4.50-4.39 (m, 2H), 4.26 (t, J=6.2 Hz, 2H), 2.52 (s, 3H), 2.38 (qd, J=6.1, 4.2 Hz, 2H).

### Example 186: 2-(5-Fluoropyridin-2-yl)-3-(1H-pyrazolo[3,4-b]pyridin-4-yl)-6,7-dihydro-5H-pyrazolo[5,1-b][1,3]oxazine

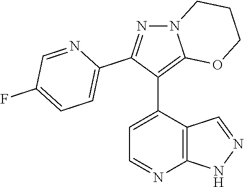 [SMILES: Fc1ccc(-c2nn3c(c2-c2ccnc4[nH]ncc24)OCCC3)nc1]

The title compound was prepared in a manner analogous to Example 1, Steps A-B, except using 3-bromo-2-(5-fluoropyridin-2-yl)-6,7-dihydro-5H-pyrazolo[5,1-b][1,3]oxazine (Intermediate 119) instead of 3-bromo-2-(5-fluoropyridin-2-yl)-6,7-dihydro-4H-pyrazolo[5,1-c][1,4]oxazine (Intermediate 37) and 4-(4,4,5,5-tetramethyl-1,3,2-dioxaborolan-2-yl)-1-((2-(trimethylsilyl)ethoxy)methyl)-1H-pyrazolo[3,4-b]pyridine (Intermediate 23) instead of 4-(4,4,5,5-tetramethyl-1,3,2-dioxaborolan-2-yl)-1-((2-(trimethylsilyl)ethoxy)methyl)-1H-pyrrolo[2,3-b]pyridine (Intermediate 21) in Step A. MS (ESI): mass calcd. for C₁₇H₁₃FN₆O, 336.3; m/z found, 337.0 [M+H]⁺. ¹H NMR (500 MHz, CD₃OD) δ 8.36 (d, J=4.9 Hz, 1H), 8.28-8.26 (m, 1H), 7.71-7.68 (m, 1H), 7.62 (td, J=8.6, 2.9 Hz, 1H), 7.52 (s, 1H), 7.02 (d, J=4.9 Hz, 1H), 4.52-4.47 (m, 2H), 4.33 (t, J=6.2 Hz, 2H), 2.42 (dt, J=11.4, 6.0 Hz, 2H).

### Example 187: 2-(5-Fluoropyridin-2-yl)-3-(6-methyl-1H-pyrazolo[3,4-b]pyridin-4-yl)-6,7-dihydro-5H-pyrazolo[5,1-b][1,3]oxazine

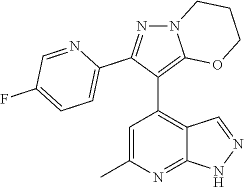 [SMILES: Cc1cc(-c2c(-c3ccc(F)cn3)nn3c2OCCC3)c2cn[nH]c2n1]

The title compound was prepared in a manner analogous to Example 1, Steps A-B, except using 3-bromo-2-(5-fluoropyridin-2-yl)-6,7-dihydro-5H-pyrazolo[5,1-b][1,3]oxazine (Intermediate 119) instead of 3-bromo-2-(5-fluoropyridin-2-yl)-6,7-dihydro-4H-pyrazolo[5,1-c][1,4]oxazine (Intermediate 37) and 6-methyl-4-(4,4,5,5-tetramethyl-1,3,2-dioxaborolan-2-yl)-1-((2-(trimethylsilyl)ethoxy)methyl)-1H-pyrazolo[3,4-b]pyridine (Intermediate 26) instead of 4-(4,4,5,5-tetramethyl-1,3,2-dioxaborolan-2-yl)-1-((2-(trimethylsilyl)ethoxy)methyl)-1H-pyrrolo[2,3-b]pyridine (Intermediate 21) in Step A. MS (ESI): mass calcd. for C₁₈H₁₅FN₆O, 350.4; m/z found, 351.0 [M+H]⁺. ¹H NMR (500 MHz, CD₃OD) δ 8.28-8.27 (m, 1H), 7.68-7.65 (m, 1H), 7.61 (td, J=8.5, 2.9 Hz, 1H), 7.36 (s, 1H), 6.94 (s, 1H), 4.50-4.45 (m, 2H), 4.32 (t, J=6.2 Hz, 2H), 2.56 (s, 3H), 2.45-2.38 (m, 2H).

### Example 188: 2-(3,5-Difluoro-2-pyridyl)-3-(1H-pyrazolo[3,4-b]pyridin-4-yl)-6,7-dihydro-5H-pyrazolo[5,1-b][1,3]oxazine

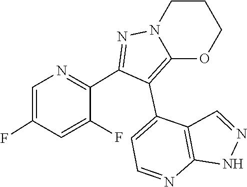 [SMILES: Fc1cnc(-c2nn3c(c2-c2ccnc4[nH]ncc24)OCCC3)c(F)c1]

The title compound was prepared in a manner analogous to Example 1, Steps A-B, except using 4-(4,4,5,5-tetramethyl-1,3,2-dioxaborolan-2-yl)-1-((2-(trimethylsilyl)ethoxy)methyl)-1H-pyrazolo[3,4-b]pyridine (Intermediate 23) instead of 4-(4,4,5,5-tetramethyl-1,3,2-dioxaborolan-2-yl)-1-((2-(trimethylsilyl)ethoxy)methyl)-1H-pyrrolo[2,3-b]pyridine (Intermediate 21) and 3-bromo-2-(3,5-difluoropyridin-2-yl)-6,7-dihydro-5H-pyrazolo[5,1-b][1,3]oxazine (Intermediate 120) instead of 3-bromo-2-(5-fluoropyridin-2-yl)-6,7-dihydro-4H-pyrazolo[5,1-c][1,4]oxazine (Intermediate 37) in Step A. MS (ESI): mass calcd. for C₁₇H₁₂F₂N₆O, 354.1; m/z found, 355.1 [M+H]⁺. ¹H NMR (500 MHz, DMSO-d₆) δ 13.46 (s, 1H), 8.47 (d, J=2.4 Hz, 1H), 8.31 (d, J=4.8 Hz, 1H), 8.05-7.92 (m, 1H), 7.54 (s, 1H), 6.75 (d, J=4.8 Hz, 1H), 4.54-4.46 (m, 2H), 4.27 (t, J=6.1 Hz, 2H), 2.39-2.31 (m, 2H).

### Example 189: 2-(3,5-Difluoro-2-pyridyl)-3-(6-methyl-1H-pyrazolo[3,4-b]pyridin-4-yl)-6,7-dihydro-5H-pyrazolo[5,1-b][1,3]oxazine

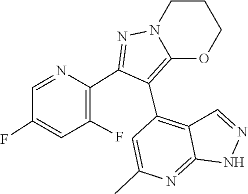 [SMILES: Cc1cc(-c2c(-c3ncc(F)cc3F)nn3c2OCCC3)c2cn[nH]c2n1]

The title compound was prepared in a manner analogous to Example 1, Steps A-B, except using 3-bromo-2-(3,5-difluoropyridin-2-yl)-6,7-dihydro-5H-pyrazolo[5,1-b][1,3]oxazine (Intermediate 120) instead of 3-bromo-2-(5-fluoropyridin-2-yl)-6,7-dihydro-4H-pyrazolo[5,1-c][1,4]oxazine (Intermediate 37) and 6-methyl-4-(4,4,5,5-tetramethyl-1,3,2-dioxaborolan-2-yl)-1-((2-(trimethylsilyl)ethoxy)methyl)-1H-pyrazolo[3,4-b]pyridine (Intermediate 26) instead of 4-(4,4,5,5-tetramethyl-1,3,2-dioxaborolan-2-yl)-1-((2-(trimethylsilyl)ethoxy)methyl)-1H-pyrrolo[2,3-b]pyridine (Intermediate 21) in Step A and HCl/dioxane instead of TFA/DCM in Step B. MS (ESI): mass calcd. for C₁₈H₁₄F₂N₆O, 368.1; m/z found, 369.1 [M+H]⁺. ¹H NMR (500 MHz, DMSO-d₆) δ 13.23 (s, 1H), 8.47 (d, J=2.4 Hz, 1H), 7.95 (ddd, J=9.9, 8.9, 2.4 Hz, 1H), 7.32 (s, 1H), 6.70 (s, 1H), 4.52-4.44 (m, 2H), 4.27 (t, J=6.1 Hz, 2H), 2.45 (s, 3H), 2.37-2.29 (m, 2H).

### Example 190: 2-(5-Fluoro-2-pyridyl)-3-(6-methoxy-1,5-naphthyridin-4-yl)-6,7-dihydro-5H-pyrazolo[5,1-b][1,3]oxazine

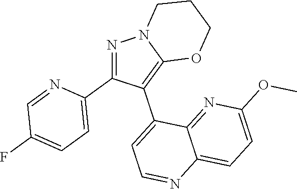 [SMILES: COc1ccc2nccc(-c3c(-c4ccc(F)cn4)nn4c3OCCC4)c2n1]

The title compound was prepared in a manner analogous to Example 1, step A, except using (3-bromo-2-(5-fluoropyridin-2-yl)-6,7-dihydro-5H-pyrazolo[5,1-b][1,3]oxazine (Intermediate 119) instead of 3-bromo-2-(5-fluoropyridin-2-yl)-6,7-dihydro-4H-pyrazolo[5,1-c][1,4]oxazine (Intermediate 37) and 2-methoxy-8-(4,4,5,5-tetramethyl-1,3,2-dioxaborolan-2-yl)-1,5-naphthyridine (Intermediate 30) instead of 4-(4,4,5,5-tetramethyl-1,3,2-dioxaborolan-2-yl)-1-((2-(trimethylsilyl)ethoxy)methyl)-1H-pyrrolo[2,3-b]pyridine (Intermediate 21). MS (ESI): mass calcd. for C₂₀H₁₆FN₅O₂, 377.1; m/z found, 378.1 [M+H]⁺. ¹H NMR (400 MHz, CD₃OD) δ 8.67 (d, J=4.7 Hz, 1H), 8.13 (d, J=9.1 Hz, 1H), 8.09-7.97 (m, 1H), 7.66 (d, J=4.6 Hz, 1H), 7.64-7.52 (m, 1H), 7.54-7.39 (m, 1H), 7.04 (d, J=9.1 Hz, 1H), 4.49-4.39 (m, 2H), 4.33 (t, J=6.2 Hz, 2H), 3.46 (s, 3H), 2.52-2.30 (m, 2H).

### Example 191: (R S)-2-(4-Fluorophenyl)-6-methyl-3-(1H-pyrazolo[3,4-b]pyridin-4-yl)-6,7-dihydro-5H-pyrazolo[5,1-b][1,3]oxazine

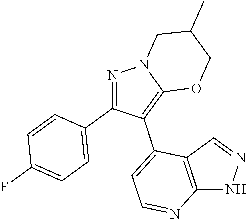 [SMILES: CC1COc2c(-c3ccnc4[nH]ncc34)c(-c3ccc(F)cc3)nn2C1]

The title compound was prepared in a manner analogous to Example 1, Steps A-B, except using 4-(4,4,5,5-tetramethyl-1,3,2-dioxaborolan-2-yl)-1-((2-(trimethylsilyl)ethoxy)methyl)-1H-pyrazolo[3,4-b]pyridine (Intermediate 23) instead of 4-(4,4,5,5-tetramethyl-1,3,2-dioxaborolan-2-yl)-1-((2-(trimethylsilyl)ethoxy)methyl)-1H-pyrrolo[2,3-b]pyridine (Intermediate 21) and 3-bromo-2-(4-fluorophenyl)-6-methyl-6,7-dihydro-5H-pyrazolo[5,1-b][1,3]oxazine (Intermediate 121) instead of 3-bromo-2-(5-fluoropyridin-2-yl)-6,7-dihydro-4H-pyrazolo[5,1-c][1,4]oxazine (Intermediate 37) in Step A. MS (ESI): mass calcd. for C₁₉H₁₆FN₅O, 349.1; m/z found, 350.1 [M+H]⁺. ¹H NMR (400 MHz, CDCl₃): δ 12.54 (s, 1H), 8.47 (d, J=4.9 Hz, 1H), 7.68 (s, 1H), 7.43-7.33 (m, 2H), 7.04-6.88 (m, 3H), 4.46-4.32 (m, 2H), 4.00 (dd, J=11.0, 9.4 Hz, 1H), 3.88 (dd, J=12.4, 9.0 Hz, 1H), 2.71-2.56 (m, 1H), 1.22 (d, J=6.8 Hz, 3H).

### Example 192: (R/S)-2-(4-Fluorophenyl)-6-methyl-3-(6-methyl-1H-pyrazolo[3,4-b]pyridin-4-yl)-6,7-dihydro-5H-pyrazolo[5,1-b][1,3]oxazine

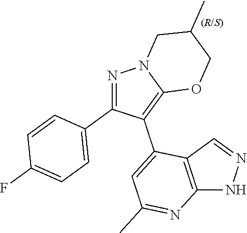 [SMILES: Cc1cc(-c2c(-c3ccc(F)cc3)nn3c2OCC(C)C3)c2cn[nH]c2n1]

The title compound was prepared in a manner analogous to Example 1, Steps A-B, except using 6-methyl-4-(4,4,5,5-tetramethyl-1,3,2-dioxaborolan-2-yl)-1-((2-(trimethylsilyl)ethoxy)methyl)-1H-pyrazolo[3,4-b]pyridine (Intermediate 26) instead of 4-(4,4,5,5-tetramethyl-1,3,2-dioxaborolan-2-yl)-1-((2-(trimethylsilyl)ethoxy)methyl)-1H-pyrrolo[2,3-b]pyridine (Intermediate 21) and 3-bromo-2-(4-fluorophenyl)-6-methyl-6,7-dihydro-5H-pyrazolo[5,1-b][1,3]oxazine (Intermediate 121) instead of 3-bromo-2-(5-fluoropyridin-2-yl)-6,7-dihydro-4H-pyrazolo[5,1-c][1,4]oxazine (Intermediate 37) in Step A. MS (ESI): mass calcd. for C₂₀H₁₈FN₅O, 363.1; m/z found, 364.1 [M+H]⁺. ¹H NMR (400 MHz, CDCl₃): δ 12.39 (s, 1H), 7.65-7.17 (m, 4H), 7.10-6.83 (m, 2H), 4.42 (s, 2H), 3.95 (dd, J=49.7, 13.2 Hz, 2H), 2.69 (s, 3H), 1.74 (s, 1H), 1.25 (s, 3H).

### Example 193: (*S)-2-(4-Fluorophenyl)-6-methyl-3-(6-methyl-1H-pyrazolo[3,4-b]pyridin-4-yl)-6,7-dihydro-5H-pyrazolo[5,1-b][1,3]oxazine

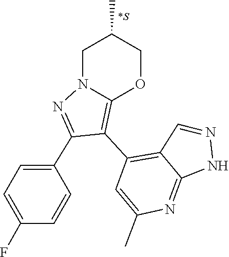 [SMILES: Cc1cc(-c2c(-c3ccc(F)cc3)nn3c2OC[C@@H](C)C3)c2cn[nH]c2n1]

The title compound was obtained after chiral separation of (R/S)-2-(4-fluorophenyl)-6-methyl-3-(6-methyl-1H-pyrazolo[3,4-b]pyridin-4-yl)-6,7-dihydro-5H-pyrazolo[5,1-b][1,3]oxazine (Example 192). The purification was performed via SFC (Stationary phase: Chiralpak AD, 5 μm 250×21 mm, Mobile phase: 35% methanol with 0.2% triethylamine, 65% CO₂) at flow rate of 2 mL/min for 12 min. and monitor at 220 nm. Second peak eluting at 7.10 min. Enantiopurity of 98.8%. MS (ESI): mass calcd. for C₂₀H₁₈FN₅O, 363.1; m/z found, 364.1 [M+H]⁺. ¹H NMR (500 MHz, DMSO-d₆) δ 13.24 (s, 1H), 7.38-7.28 (m, 3H), 7.16-7.04 (m, 2H), 6.81 (s, 1H), 4.45-4.38 (m, 1H), 4.31 (dd, J=12.0, 5.4 Hz, 1H), 4.07 (t, J=10.0 Hz, 1H), 3.85 (dd, J=12.0, 8.8 Hz, 1H), 3.36-3.27 (m, 4H), 1.09 (d, J=6.7 Hz, 3H).

### Example 194: (*R)-2-(4-Fluorophenyl)-6-methyl-3-(6-methyl-1H-pyrazolo[3,4-b]pyridin-4-yl)-6,7-dihydro-5H-pyrazolo[5,1-b][1,3]oxazine

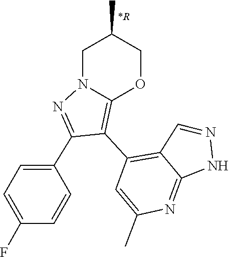 [SMILES: Cc1cc(-c2c(-c3ccc(F)cc3)nn3c2OC[C@H](C)C3)c2cn[nH]c2n1]

The title compound was obtained after chiral separation of (R/S)-2-(4-fluorophenyl)-6-methyl-3-(6-methyl-1H-pyrazolo[3,4-b]pyridin-4-yl)-6,7-dihydro-5H-pyrazolo[5,1-b][1,3]oxazine (Example 192). The purification was performed via SFC (Stationary phase: Chiralpak AD, 5 μm 250×21 mm, Mobile phase: 35% methanol with 0.2% triethylamine, 65% CO₂) at flow rate of 2 mL/min for 12 min. and monitor at 220 nm. First peak eluting at 5.29 min. Enantiopurity of 98.7%. MS (ESI): mass calcd. for C₂₀H₁₈FN₅O, 363.1; m/z found, 364.1 [M+H]⁺. ¹H NMR (500 MHz, DMSO-d₆) δ 13.24 (s, 1H), 7.36-7.30 (m, 3H), 7.15-7.07 (m, 2H), 6.81 (s, 1H), 4.44-4.38 (m, 1H), 4.34-4.28 (m, 1H), 4.06 (dd, J=10.7, 9.3 Hz, 1H), 3.85 (dd, J=12.1, 8.8 Hz, 1H), 3.35-3.26 (m, 4H), 1.09 (d, J=6.8 Hz, 3H).

### Example 195: 2-(5-Fluoropyridin-2-yl)-6-methyl-3-(thieno[3,2-b]pyridin-7-yl)-6,7-dihydro-5H-pyrazolo[5,1-b][1,3]oxazine

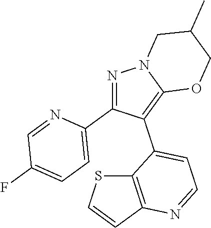 [SMILES: CC1COc2c(-c3ccnc4ccsc34)c(-c3ccc(F)cn3)nn2C1]

The title compound was prepared in a manner analogous to Example 1, step A, except using 3-bromo-2-(5-fluoropyridin-2-yl)-6-methyl-6,7-dihydro-5H-pyrazolo[5,1-b][1,3]oxazine (Intermediate 122) instead of 3-bromo-2-(5-fluoropyridin-2-yl)-6,7-dihydro-4H-pyrazolo[5,1-c][1,4]oxazine (Intermediate 37) and 7-(4,4,5,5-tetramethyl-1,3,2-dioxaborolan-2-yl)thieno[3,2-b]pyridine (Intermediate 29) instead of 4-(4,4,5,5-tetramethyl-1,3,2-dioxaborolan-2-yl)-1-((2-(trimethylsilyl)ethoxy)methyl)-1H-pyrrolo[2,3-b]pyridine (Intermediate 21). MS (ESI): mass calcd. for C₁₉H₁₅FN₄OS, 366.1; m/z found, 367.1 [M+H]⁺. ¹H NMR (400 MHz, CD₃OD) δ 8.54 (d, J=5.0 Hz, 1H), 8.17 (d, J=2.9 Hz, 1H), 7.85 (d, J=5.6 Hz, 1H), 7.69 (dd, J=8.8, 4.5 Hz, 1H), 7.56 (td, J=8.6, 2.9 Hz, 1H), 7.45 (d, J=5.6 Hz, 1H), 7.28 (d, J=5.0 Hz, 1H), 4.47-4.33 (m, 2H), 4.07 (dd, J=11.0, 9.1 Hz, 1H), 3.92 (dd, J=12.3, 8.8 Hz, 1H), 2.71-2.50 (m, 1H), 1.19 (d, J=6.8 Hz, 3H).

### Example 196: 2-(5-Fluoropyridin-2-yl)-6-methyl-3-(1H-pyrazolo[3,4-b]pyridin-4-yl)-6,7-dihydro-5H-pyrazolo[5,1-b][1,3]oxazine

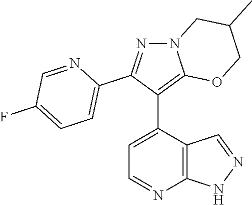 [SMILES: CC1COc2c(-c3ccnc4[nH]ncc34)c(-c3ccc(F)cn3)nn2C1]

The title compound was prepared in a manner analogous to Example 1, Steps A-B, except using 3-bromo-2-(5-fluoropyridin-2-yl)-6-methyl-6,7-dihydro-5H-pyrazolo[5,1-b][1,3]oxazine-methane (1/1) (Intermediate 122) instead of 3-bromo-2-(5-fluoropyridin-2-yl)-6,7-dihydro-4H-pyrazolo[5,1-c][1,4]oxazine (Intermediate 37) and 4-(4,4,5,5-tetramethyl-1,3,2-dioxaborolan-2-yl)-1-((2-(trimethylsilyl)ethoxy)methyl)-1H-pyrazolo[3,4-b]pyridine (Intermediate 23) instead of 4-(4,4,5,5-tetramethyl-1,3,2-dioxaborolan-2-yl)-1-((2-(trimethylsilyl)ethoxy)methyl)-1H-pyrrolo[2,3-b]pyridine (Intermediate 21) in Step A. MS (ESI): mass calcd. for C₁₈H₁₅FN₆O, 350.4; m/z found, 351.0 [M+H]⁺. ¹H NMR (500 MHz, CD₃OD) δ 8.40 (d, J=4.9 Hz, 1H), 8.32 (d, J=2.9 Hz, 1H), 7.76-7.71 (m, 1H), 7.66 (td, J=8.5, 2.9 Hz, 1H), 7.56 (s, 1H), 7.06 (d, J=4.9 Hz, 1H), 4.54-4.49 (m, 1H), 4.46-4.41 (m, 1H), 4.15 (dd, J=10.8, 9.3 Hz, 1H), 3.96 (dd, J=12.2, 8.9 Hz, 1H), 2.74-2.60 (m, 1H), 1.24 (d, J=6.9 Hz, 3H).

### Example 197: 2-(5-Fluoropyridin-2-yl)-6-methyl-3-(6-methyl-1H-pyrazolo[3,4-b]pyridin-4-yl)-6,7-dihydro-5H-pyrazolo[5,1-b][1,3]oxazine

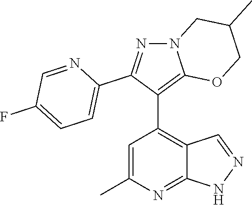 [SMILES: Cc1cc(-c2c(-c3ccc(F)cn3)nn3c2OCC(C)C3)c2cn[nH]c2n1]

The title compound was prepared in a manner analogous to Example 1, Steps A-B, except using 3-bromo-2-(5-fluoropyridin-2-yl)-6-methyl-6,7-dihydro-5H-pyrazolo[5,1-b][1,3]oxazine-methane (1/1) (Intermediate 122) instead of 3-bromo-2-(5-fluoropyridin-2-yl)-6,7-dihydro-4H-pyrazolo[5,1-c][1,4]oxazine (Intermediate 37) and 6-methyl-4-(4,4,5,5-tetramethyl-1,3,2-dioxaborolan-2-yl)-1-((2-(trimethylsilyl)ethoxy)methyl)-1H-pyrazolo[3,4-b]pyridine (Intermediate 26) instead of 4-(4,4,5,5-tetramethyl-1,3,2-dioxaborolan-2-yl)-1-((2-(trimethylsilyl)ethoxy)methyl)-1H-pyrrolo[2,3-b]pyridine (Intermediate 21) in Step A. MS (ESI): mass calcd. for C₁₉H₁₇FN₆O, 364.4; m/z found, 365.1 [M+H]⁺. ¹H NMR (500 MHz, CD₃OD) δ 8.34-8.31 (m, 1H), 7.74-7.62 (m, 2H), 7.40 (s, 1H), 6.98 (s, 1H), 4.53-4.49 (m, 1H), 4.46-4.40 (m, 1H), 4.17-4.12 (m, 1H), 3.98-3.92 (m, 1H), 2.71-2.62 (m, 1H), 2.60 (s, 3H), 1.24 (d, J=6.8 Hz, 3H).

### Example 198: (*S)-2-(5-Fluoro-2-pyridyl)-5-methyl-3-(1H-pyrazolo[3,4-b]pyridin-4-yl)-6,7-dihydro-5H-pyrazolo[5,1-b][1,3]oxazine

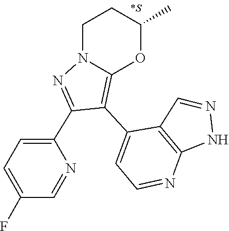 [SMILES: C[C@H]1CCn2nc(-c3ccc(F)cn3)c(-c3ccnc4[nH]ncc34)c2O1]

Step A: (Racemic)-2-(5-Fluoro-2-pyridyl)-5-methyl-3-(1H-pyrazolo[3,4-b]pyridin-4-yl)-6,7-dihydro-5H-pyrazolo[5,1-b][1,3]oxazine. The title compound was prepared in a manner analogous to Example 1, Steps A-B, except using 3-bromo-2-(5-fluoropyridin-2-yl)-5-methyl-6,7-dihydro-5H-pyrazolo[5,1-b][1,3]oxazine (Intermediate 123) instead of 3-bromo-2-(5-fluoropyridin-2-yl)-6,7-dihydro-4H-pyrazolo[5,1-c][1,4]oxazine (Intermediate 37) and 4-(4,4,5,5-tetramethyl-1,3,2-dioxaborolan-2-yl)-1-((2-(trimethylsilyl)ethoxy)methyl)-1H-pyrazolo[3,4-b]pyridine (Intermediate 23) instead of 4-(4,4,5,5-tetramethyl-1,3,2-dioxaborolan-2-yl)-1-((2-(trimethylsilyl)ethoxy)methyl)-1H-pyrrolo[2,3-b]pyridine (Intermediate 21) in Step A.

Step B: (*S)-2-(5-Fluoro-2-pyridyl)-5-methyl-3-(1H-pyrazolo[3,4-b]pyridin-4-yl)-6,7-dihydro-5H-pyrazolo[5,1-b][1,3]oxazine. (Racemic)-2-(5-Fluoro-2-pyridyl)-5-methyl-3-(1H-pyrazolo[3,4-b]pyridin-4-yl)-6,7-dihydro-5H-pyrazolo[5,1-b][1,3]oxazine was further purified by SFC Method D. The pure fractions were collected, and the volatiles were removed under reduced pressure. The product was suspended in water (10 mL), the mixture frozen using dry ice/ethanol, and then lyophilized to dryness to afford the title compound (62.1 mg, 31%) as a white solid. LC-MS (ESI): mass calcd. for C₁₈H₁₅FN₆O 350.13 m/z, found 351.0 [M+H]⁺. ¹H NMR (400 MHz, CDCl₃): δ 11.29 (br s, 1H), 8.46 (d, J=5.2 Hz, 1H), 8.39 (d, J=2.8 Hz, 1H), 7.62 (s, 1H), 7.47 (dd, J=4.8, 8.8 Hz, 1H), 7.35-7.29 (m, 1H), 7.03 (d, J=5.2 Hz, 1H), 4.56-4.49 (m, 1H), 4.44-4.38 (m, 1H), 4.34-4.26 (m, 1H), 2.37-2.29 (m, 1H), 2.28-2.19 (m, 1H), 1.53 (d, J=6.4 Hz, 3H).

### Example 199: (*R)-2-(5-Fluoro-2-pyridyl)-5-methyl-3-(1H-pyrazolo[3,4-b]pyridin-4-yl)-6,7-dihydro-5H-pyrazolo[5,1-b][1,3]oxazine

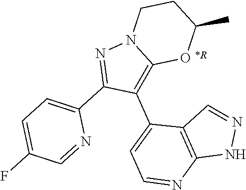 [SMILES: C[C@@H]1CCn2nc(-c3ccc(F)cn3)c(-c3ccnc4[nH]ncc34)c2O1]

The title compound was isolated (27.5 mg, 13%) as a white solid from SFC purification of Example 198, LC-MS (ESI): mass calcd. for C₁₈H₁₅FN₆O 350.13 m/z, found 351.0 [M+H]⁺. ¹H NMR (400 MHz, CDCl₃): δ 11.37 (br s, 1H), 8.46 (d, J=4.8 Hz, 1H), 8.39 (d, J=2.8 Hz, 1H), 7.63 (s, 1H), 7.47 (dd, J=4.8, 8.8 Hz, 1H), 7.35-7.29 (m, 1H), 7.03 (d, J=5.2 Hz, 1H), 4.56-4.48 (m, 1H), 4.44-4.38 (m, 1H), 4.34-4.26 (m, 1H), 2.37-2.29 (m, 1H), 2.28-2.19 (m, 1H), 1.53 (d, J=6.4 Hz, 3H).

### Example 200: (*S)-2-(5-Fluoro-2-pyridyl)-5-methyl-3-(6-methyl-1H-pyrazolo[3,4-b]pyridin-4-yl)-6,7-dihydro-5H-pyrazolo[5,1-b][1,3]oxazine

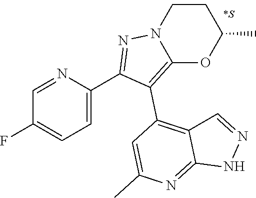 [SMILES: Cc1cc(-c2c(-c3ccc(F)cn3)nn3c2O[C@@H](C)CC3)c2cn[nH]c2n1]

Step A: (Racemic)-2-(5-Fluoro-2-pyridyl)-5-methyl-3-(6-methyl-1H-pyrazolo[3,4-b]pyridin-4-yl)-6,7-dihydro-5H-pyrazolo[5,1-b][1,3]oxazine The title compound was prepared in a manner analogous to Example 1, Steps A-B, except using 3-bromo-2-(5-fluoropyridin-2-yl)-5-methyl-6,7-dihydro-5H-pyrazolo[5,1-b][1,3]oxazine (Intermediate 123) instead of 3-bromo-2-(5-fluoropyridin-2-yl)-6,7-dihydro-4H-pyrazolo[5,1-c][1,4]oxazine (Intermediate 37) and 6-Methyl-4-(4,4,5,5-tetramethyl-1,3,2-dioxaborolan-2-yl)-1-((2-(trimethylsilyl)ethoxy)methyl)-1H-pyrazolo[3,4-b]pyridine. (Intermediate 26) instead of 4-(4,4,5,5-tetramethyl-1,3,2-dioxaborolan-2-yl)-1-((2-(trimethylsilyl)ethoxy)methyl)-1H-pyrrolo[2,3-b]pyridine (Intermediate 21) in Step A.

Step B: (*S)-2-(5-Fluoro-2-pyridyl)-5-methyl-3-(6-methyl-1H-pyrazolo[3,4-b]pyridin-4-yl)-6,7-dihydro-5H-pyrazolo[5,1-b][1,3]oxazine. (Racemic)-2-(5-Fluoro-2-pyridyl)-5-methyl-3-(6-methyl-1H-pyrazolo[3,4-b]pyridin-4-yl)-6,7-dihydro-5H-pyrazolo[5,1-b][1,3]oxazine was further purified by SFC Method C. The pure fractions were collected and the volatiles were removed under reduced pressure. The product was suspended in water (10 mL), the mixture frozen using dry ice/ethanol, and then lyophilized to dryness to afford the title compound (54.0 mg, 42%) as a white solid. LC-MS (ESI): mass calcd. for C₁₉H₁₇FN₆O 364.15 m/z, found 365.0 [M+H]⁺. ¹H NMR (400 MHz, CDCl₃): δ 11.27 (br s, 1H), 8.39 (d, J=2.8 Hz, 1H), 7.49 (s, 1H), 7.46 (dd, J=4.4, 8.8 Hz, 1H), 7.34-7.28 (m, 1H), 6.94 (s, 1H), 4.55-4.48 (m, 1H), 4.43-4.37 (m, 1H), 4.33-4.26 (m, 1H), 2.65 (s, 3H), 2.36-2.29 (m, 1H), 2.27-2.18 (m, 1H), 1.53 (d, J=6.4 Hz, 3H).

### Example 201: (*R)-2-(5-Fluoro-2-pyridyl)-5-methyl-3-(6-methyl-1H-pyrazolo[3,4-b]pyridin-4-yl)-6,7-dihydro-5H-pyrazolo[5,1-b][1,3]oxazine

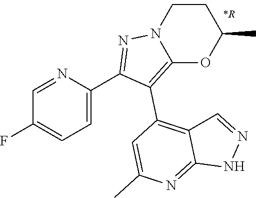 [SMILES: Cc1cc(-c2c(-c3ccc(F)cn3)nn3c2O[C@H](C)CC3)c2cn[nH]c2n1]

The title compound was prepared in a manner analogous to Example 1, Steps A-B, except using 3-bromo-2-(5-fluoropyridin-2-yl)-5-methyl-6,7-dihydro-5H-pyrazolo[5,1-b][1,3]oxazine (Intermediate 123) instead of 3-bromo-2-(5-fluoropyridin-2-yl)-6,7-dihydro-4H-pyrazolo[5,1-c][1,4]oxazine (Intermediate 37) and 6-Methyl-4-(4,4,5,5-tetramethyl-1,3,2-dioxaborolan-2-yl)-1-((2-(trimethylsilyl)ethoxy)methyl)-1H-pyrazolo[3,4-b]pyridine. (Intermediate 26) instead of 4-(4,4,5,5-tetramethyl-1,3,2-dioxaborolan-2-yl)-1-((2-(trimethylsilyl)ethoxy)methyl)-1H-pyrrolo[2,3-b]pyridine (Intermediate 21) in Step A. The product of Step B was further purified by SFC Method C. The pure fractions were collected and the volatiles were removed under reduced pressure. The product was suspended in water (10 mL), the mixture frozen using dry ice/ethanol, and then lyophilized to dryness to afford the title compound (50.9 mg, 39%) as a white solid. LC-MS (ESI): mass calcd. for C₁₉H₁₇FN₆O 364.15 m/z, found 365.0 [M+H]⁺. ¹H NMR (400 MHz, CDCl₃): δ 11.39 (br s, 1H), 8.39 (d, J=2.8 Hz, 1H), 7.49 (s, 1H), 7.46 (dd, J=4.4, 8.8 Hz, 1H), 7.34-7.28 (m, 1H), 6.94 (s, 1H), 4.56-4.48 (m, 1H), 4.44-4.37 (m, 1H), 4.34-4.26 (m, 1H), 2.65 (s, 3H), 2.36-2.29 (m, 1H), 2.27-2.16 (m, 1H), 1.53 (d, J=6.4 Hz, 3H).

### Example 202: (5*S,7*R)-2-(5-Fluoro-2-pyridyl)-5,7-dimethyl-3-(1H-pyrazolo[3,4-b]pyridin-4-yl)-6,7-dihydro-5H-pyrazolo[5,1-b][1,3]oxazine

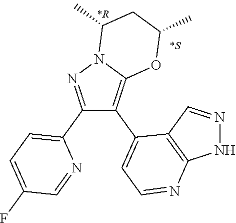 [SMILES: C[C@@H]1C[C@H](C)Oc2c(-c3ccnc4[nH]ncc34)c(-c3ccc(F)cn3)nn21]

The title compound was prepared in a manner analogous to Example 1, Steps A-B, except using cis-3-bromo-2-(5-fluoropyridin-2-yl)-5,7-dimethyl-6,7-dihydro-5H-pyrazolo[5,1-b][1,3]oxazine (Intermediate 124) instead of 3-bromo-2-(5-fluoropyridin-2-yl)-6,7-dihydro-4H-pyrazolo[5,1-c][1,4]oxazine (Intermediate 37) and 4-(4,4,5,5-tetramethyl-1,3,2-dioxaborolan-2-yl)-1-((2-(trimethylsilyl)ethoxy)methyl)-1H-pyrazolo[3,4-b]pyridine (Intermediate 23) instead of 4-(4,4,5,5-tetramethyl-1,3,2-dioxaborolan-2-yl)-1-((2-(trimethylsilyl)ethoxy)methyl)-1H-pyrrolo[2,3-b]pyridine (Intermediate 21) and Pd(dppf)Cl₂ instead of CataCXium® A Pd G3 in Step A. The product of Step B was further purified by SFC Method D to afford pure products. The pure fractions were collected and the volatiles were removed under reduced pressure. The product was suspended in water (10 mL) and CH₃CN (10 mL), the mixture frozen using dry ice/EtOH, and then lyophilized to dryness to afford the title compound (25.5 mg, 26%). LCMS (ESI): mass calcd. for C₁₉H₁₇FN₆O 364.14 m/z, found 365.0 [M+H]⁺. ¹H NMR (400 MHz, CDCl₃) δ 11.03 (br s, 1H), 8.45 (d, J=4.8 Hz, 1H), 8.39 (d, J=2.8 Hz, 1H), 7.63 (s, 1H), 7.52 (dd, J=4.0, 8.4 Hz, 1H), 7.34 (dt, J=2.8, 8.4 Hz, 1H), 7.02 (d, J=4.8 Hz, 1H), 4.52-4.37 (m, 2H), 2.36 (dd, J=5.4, 12.4 Hz, 1H), 2.05-1.93 (m, 1H), 1.75 (d, J=6.4 Hz, 3H), 1.52 (d, J=6.0 Hz, 3H).

### Example 203: (5*R,7*S)-2-(5-Fluoro-2-pyridyl)-5,7-dimethyl-3-(1H-pyrazolo[3,4-b]pyridin-4-yl)-6,7-dihydro-5H-pyrazolo[5,1-b][1,3]oxazine

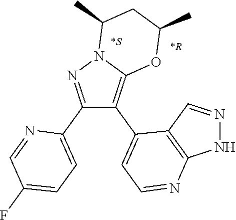 [SMILES: C[C@@H]1C[C@H](C)n2nc(-c3ccc(F)cn3)c(-c3ccnc4[nH]ncc34)c2O1]

The title compound was prepared in a manner analogous to Example 1, Steps A-B, except using cis-3-bromo-2-(5-fluoropyridin-2-yl)-5,7-dimethyl-6,7-dihydro-5H-pyrazolo[5,1-b][1,3]oxazine (Intermediate 124) instead of 3-bromo-2-(5-fluoropyridin-2-yl)-6,7-dihydro-4H-pyrazolo[5,1-c][1,4]oxazine (Intermediate 37) and 4-(4,4,5,5-tetramethyl-1,3,2-dioxaborolan-2-yl)-1-((2-(trimethylsilyl)ethoxy)methyl)-1H-pyrazolo[3,4-b]pyridine (Intermediate 23) instead of 4-(4,4,5,5-tetramethyl-1,3,2-dioxaborolan-2-yl)-1-((2-(trimethylsilyl)ethoxy)methyl)-1H-pyrrolo[2,3-b]pyridine (Intermediate 21) and Pd(dppf)Cl₂ instead of CataCXium® A Pd G3 in Step A. The product of Step B was further purified by SFC Method D to afford pure products. The pure fractions were collected and the volatiles were removed under reduced pressure. The product was suspended in water (10 mL) and CH₃CN (10 mL), the mixture frozen using dry ice/EtOH, and then lyophilized to dryness to afford the title compound (33.8 mg, 34%) as a white solid. LCMS (ESI): mass calcd. for C₁₉H₁₇FN₆O 364.14 m/z, found 365.1 [M+H]⁺. ¹H NMR (400 MHz, CDCl₃) δ 11.22 (br s, 1H), 8.45 (d, J=4.8 Hz, 1H), 8.39 (d, J=2.8 Hz, 1H), 7.63 (s, 1H), 7.52 (dd, J=4.4, 8.8 Hz, 1H), 7.33 (dt, J=2.8, 8.4 Hz, 1H), 7.02 (d, J=4.8 Hz, 1H), 4.56-4.38 (m, 2H), 2.36 (br dd, J=5.4, 12.4 Hz, 1H), 2.06-1.91 (m, 1H), 1.75 (br d, J=6.4 Hz, 3H), 1.52 (br d, J=6.4 Hz, 3H).

### Example 204: (5*R,7*R)-2-(S-Fluoropyridin-2-yl)-5,7-dimethyl-3-(1H-pyrazolo[3,4-b]pyridin-4-yl)-6,7-dihydro-5H-pyrazolo[5,1-b][1,3]oxazine

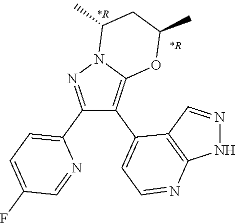 [SMILES: C[C@@H]1C[C@@H](C)n2nc(-c3ccc(F)cn3)c(-c3ccnc4[nH]ncc34)c2O1]

The title compound was prepared in a manner analogous to Example 1, Steps A-B, except using trans-3-bromo-2-(5-fluoropyridin-2-yl)-5,7-dimethyl-6,7-dihydro-5H-pyrazolo[5,1-b][1,3]oxazine (Intermediate 125) instead of 3-bromo-2-(5-fluoropyridin-2-yl)-6,7-dihydro-4H-pyrazolo[5,1-c][1,4]oxazine (Intermediate 37) and 4-(4,4,5,5-tetramethyl-1,3,2-dioxaborolan-2-yl)-1-((2-(trimethylsilyl)ethoxy)methyl)-1H-pyrazolo[3,4-b]pyridine (Intermediate 23) instead of 4-(4,4,5,5-tetramethyl-1,3,2-dioxaborolan-2-yl)-1-((2-(trimethylsilyl)ethoxy)methyl)-1H-pyrrolo[2,3-b]pyridine (Intermediate 21) in Step A. The product of Step B was further purified by SFC Method C; to afford pure products. The pure fractions were collected and the volatiles were removed under reduced pressure. The product was suspended in water (20 mL) and CH₃CN (20 mL), the mixture frozen using dry ice/EtOH, and then lyophilized to dryness to afford the title compound (26.2 mg, 35%) as a white solid. LCMS (ESI): mass calcd. for C₁₉H₁₇FN₆O 364.38 m/z found 365.0 [M+H]⁺. ¹H NMR (400 MHz, CDCl₃) δ 11.32 (br s, 1H), 8.56-8.31 (m, 2H), 7.62 (s, 1H), 7.51 (dd, J=4.4, 8.8 Hz, 1H), 7.34 (dt, J=2.8, 8.4 Hz, 1H), 7.03 (d, J=4.8 Hz, 1H), 4.71-4.58 (m, 2H), 2.38-2.26 (m, 1H), 2.07 (br d, J=14.4 Hz, 1H), 1.72 (d, J=6.8 Hz, 3H), 1.53 (d, J=6.0 Hz, 3H).

### Example 205: (5*S,7*S)-2-(5-Fluoropyridin-2-yl)-5,7-dimethyl-3-(1H-pyrazolo[3,4-b]pyridin-4-yl)-6,7-dihydro-5H-pyrazolo[5,1-b][1,3]oxazine

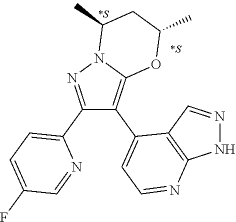 [SMILES: C[C@H]1C[C@H](C)n2nc(-c3ccc(F)cn3)c(-c3ccnc4[nH]ncc34)c2O1]

The title compound was prepared in a manner analogous to Example 1, Steps A-B, except using trans-3-bromo-2-(5-fluoropyridin-2-yl)-5,7-dimethyl-6,7-dihydro-5H-pyrazolo[5,1-b][1,3]oxazine (Intermediate 125) instead of 3-bromo-2-(5-fluoropyridin-2-yl)-6,7-dihydro-4H-pyrazolo[5,1-c][1,4]oxazine (Intermediate 37) and 4-(4,4,5,5-tetramethyl-1,3,2-dioxaborolan-2-yl)-1-((2-(trimethylsilyl)ethoxy)methyl)-1H-pyrazolo[3,4-b]pyridine (Intermediate 23) instead of 4-(4,4,5,5-tetramethyl-1,3,2-dioxaborolan-2-yl)-1-((2-(trimethylsilyl)ethoxy)methyl)-1H-pyrrolo[2,3-b]pyridine (Intermediate 21) in Step A. The product of Step B was further purified by SFC Method C; to afford pure products. The pure fractions were collected and the volatiles were removed under reduced pressure. The product was suspended in water (20 mL) and CH₃CN (20 mL), the mixture frozen using dry ice/EtOH, and then lyophilized to dryness to afford the title compound (26.6 mg, 35%) as a white solid. LCMS (ESI): mass calcd. for C₁₉H₁₇FN₆O 364.38 m/z found 365.1 [M+H]⁺. ¹H NMR (400 MHz, CDCl₃) δ 11.17 (br s, 1H), 8.57-8.31 (m, 2H), 7.62 (s, 1H), 7.51 (br dd, J=4.4, 8.8 Hz, 1H), 7.37-7.30 (m, 1H), 7.03 (br d, J=5.2 Hz, 1H), 4.63 (br d, J=4.4 Hz, 2H), 2.39-2.26 (m, 1H), 2.12-2.01 (m, 1H), 1.72 (br d, J=6.8 Hz, 3H), 1.53 (br d, J=6.0 Hz, 3H).

### Example 206: (5*S,7*R)-2-(S-Fluoro-2-pyridyl)-5,7-dimethyl-3-(6-methyl-1H-pyrazolo[3,4-b]pyridin-4-yl)-6,7-dihydro-5H-pyrazolo[5,1-b][1,3]oxazine

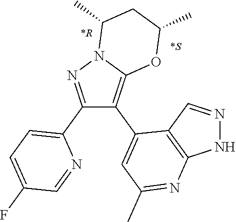 [SMILES: Cc1cc(-c2c(-c3ccc(F)cn3)nn3c2O[C@@H](C)C[C@H]3C)c2cn[nH]c2n1]

The title compound was prepared in a manner analogous to Example 1, Steps A-B, except using cis-3-bromo-2-(5-fluoropyridin-2-yl)-5,7-dimethyl-6,7-dihydro-5H-pyrazolo[5,1-b][1,3]oxazine (Intermediate 124) instead of 3-bromo-2-(5-fluoropyridin-2-yl)-6,7-dihydro-4H-pyrazolo[5,1-c][1,4]oxazine (Intermediate 37) and 6-Methyl-4-(4,4,5,5-tetramethyl-1,3,2-dioxaborolan-2-yl)-1-((2-(trimethylsilyl)ethoxy)methyl)-1H-pyrazolo[3,4-b]pyridine (Intermediate 26) instead of 4-(4,4,5,5-tetramethyl-1,3,2-dioxaborolan-2-yl)-1-((2-(trimethylsilyl)ethoxy)methyl)-1H-pyrrolo[2,3-b]pyridine (Intermediate 21) in Step A. The product of Step B was further purified by SFC Method H; to afford pure products. The pure fractions were collected and the volatiles were removed under reduced pressure. The product was suspended in water (20 mL) and CH₃CN (20 mL), the mixture frozen using dry ice/EtOH, and then lyophilized to dryness to afford the title compound (62.7 mg, 36%) as a white solid. LCMS (ESI): mass calcd. for C₂₀H₁₉FN₆O 378.16 m/z, found 379.1 [M+H]⁺. ¹H NMR (400 MHz, CDCl₃) δ 10.81 (br s, 1H), 8.39 (d, J=3.2 Hz, 1H), 7.54-7.51 (m, 1H), 7.50 (s, 1H), 7.35-7.31 (m, 1H), 6.93 (s, 1H), 4.45-4.40 (m, 2H), 2.63 (s, 3H), 2.40-2.30 (m, 1H), 2.06-1.92 (m, 1H), 1.75 (d, J=6.4 Hz, 3H), 1.52 (d, J=6.4 Hz, 3H).

### Example 207: (5*R,7*S)-2-(5-Fluoro-2-pyridyl)-5,7-dimethyl-3-(6-methyl-H-pyrazolo[3,4-b]pyridin-4-yl)-6,7-dihydro-5H-pyrazolo[5,1-b][1,3]oxazine

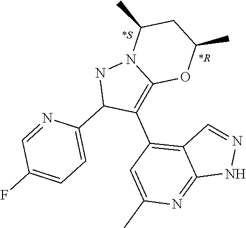

The title compound was prepared in a manner analogous to Example 1, Steps A-B, except using cis-3-bromo-2-(5-fluoropyridin-2-yl)-5,7-dimethyl-6,7-dihydro-5H-pyrazolo[5,1-b][1,3]oxazine (Intermediate 124) instead of 3-bromo-2-(5-fluoropyridin-2-yl)-6,7-dihydro-4H-pyrazolo[5,1-c][1,4]oxazine (Intermediate 37) and 6-Methyl-4-(4,4,5,5-tetramethyl-1,3,2-dioxaborolan-2-yl)-1-((2-(trimethylsilyl)ethoxy)methyl)-1H-pyrazolo[3,4-b]pyridine (Intermediate 26) instead of 4-(4,4,5,5-tetramethyl-1,3,2-dioxaborolan-2-yl)-1-((2-(trimethylsilyl)ethoxy)methyl)-1H-pyrrolo[2,3-b]pyridine (Intermediate 21) in Step A. The product of Step B was further purified by SFC Method H; to afford pure products. The pure fractions were collected and the volatiles were removed under reduced pressure. The product was suspended in water (20 mL) and CH₃CN (20 mL), the mixture frozen using dry ice/EtOH, and then lyophilized to dryness to afford a white solid (72 mg, crude), which was then subjected to preparative HPLC Method I: to afford pure product. The product was suspended in water (10 mL), the mixture frozen using dry ice/EtOH, and then lyophilized to dryness to afford the title compound (55.6 mg, 34%) as a white solid. LCMS (ESI): mass calcd. for C₂₀H₁₉FN₆O 378.16 m/z, found 379.0 [M+H]⁺. ¹H NMR (400 MHz, CDCl₃) δ 8.39 (d, J=2.8 Hz, 1H), 7.54-7.50 (m, 1H), 7.50 (s, 1H), 7.35-7.33 (m, 1H), 6.93 (s, 1H), 4.50-4.40 (m, 2H), 2.64 (s, 3H), 2.36-2.33 (m, 1H), 2.04-1.91 (m, 1H), 1.75 (br d, J=6.4 Hz, 3H), 1.52 (d, J=6.4 Hz, 3H).

### Example 208: (5*R,7*R)-2-(5-Fluoro-2-pyridyl)-5,7-dimethyl-3-(6-methyl-1H-pyrazolo[3,4-b]pyridin-4-yl)-6,7-dihydro-5H-pyrazolo[5,1-b][1,3]oxazine

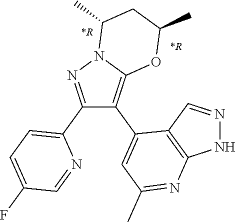 [SMILES: Cc1cc(-c2c(-c3ccc(F)cn3)nn3c2O[C@H](C)C[C@H]3C)c2cn[nH]c2n1]

The title compound was prepared in a manner analogous to Example 1, Steps A-B, except using trans-3-bromo-2-(5-fluoropyridin-2-yl)-5,7-dimethyl-6,7-dihydro-5H-pyrazolo[5,1-b][1,3]oxazine (Intermediate 125) instead of 3-bromo-2-(5-fluoropyridin-2-yl)-6,7-dihydro-4H-pyrazolo[5,1-c][1,4]oxazine (Intermediate 37) and 6-methyl-4-(4,4,5,5-tetramethyl-1,3,2-dioxaborolan-2-yl)-1-((2-(trimethylsilyl)ethoxy)methyl)-1H-pyrazolo[3,4-b]pyridine (Intermediate 26) instead of 4-(4,4,5,5-tetramethyl-1,3,2-dioxaborolan-2-yl)-1-((2-(trimethylsilyl)ethoxy)methyl)-1H-pyrrolo[2,3-b]pyridine (Intermediate 21) in Step A. The product of Step B was further purified by SFC Method D. The product was suspended in water (10 mL)/CH₃CN (10 mL), the mixture frozen using dry ice/ethanol, and then lyophilized to dryness to afford the title compound (32.8 mg, 24%) as a white solid. LC-MS (ESI): mass calcd. for C₂₀H₁₉FN₆O 378.16 m/z, found 379.1 [M+H]⁺. ¹H NMR (400 MHz, CDCl₃) δ 11.15 (br s, 1H), 8.39 (d, J=2.8 Hz, 1H), 7.54-7.47 (m, 2H), 7.33 (dt, J=3.2, 8.4 Hz, 1H), 6.94 (s, 1H), 4.70-4.57 (m, 2H), 2.65 (s, 3H), 2.36-2.26 (m, 1H), 2.07 (td, J=2.8, 14.4 Hz, 1H), 1.72 (d, J=6.8 Hz, 3H), 1.53 (d, J=6.4 Hz, 3H).

### Example 209: (5*S,7*S)-2-(5-Fluoro-2-pyridyl)-5,7-dimethyl-3-(6-methyl-1H-pyrazolo[3,4-b]pyridin-4-yl)-6,7-dihydro-5H-pyrazolo[5,1-b][1,3]oxazine

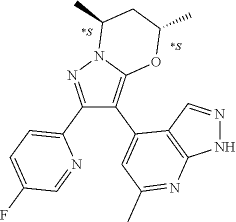 [SMILES: Cc1cc(-c2c(-c3ccc(F)cn3)nn3c2O[C@@H](C)C[C@@H]3C)c2cn[nH]c2n1]

The title compound was prepared in a manner analogous to Example 1, Steps A-B, except using trans-3-bromo-2-(5-fluoropyridin-2-yl)-5,7-dimethyl-6,7-dihydro-5H-pyrazolo[5,1-b][1,3]oxazine (Intermediate 125) instead of 3-bromo-2-(5-fluoropyridin-2-yl)-6,7-dihydro-4H-pyrazolo[5,1-c][1,4]oxazine (Intermediate 37) and 6-Methyl-4-(4,4,5,5-tetramethyl-1,3,2-dioxaborolan-2-yl)-1-((2-(trimethylsilyl)ethoxy)methyl)-1H-pyrazolo[3,4-b]pyridine (Intermediate 26) instead of 4-(4,4,5,5-tetramethyl-1,3,2-dioxaborolan-2-yl)-1-((2-(trimethylsilyl)ethoxy)methyl)-1H-pyrrolo[2,3-b]pyridine (Intermediate 21) in Step A. The product of Step B was further purified by SFC Method D. The product was suspended in water (10 mL)/CH₃CN (10 mL), the mixture frozen using dry ice/ethanol, and then lyophilized to dryness to afford the title compound (33.3 mg, 25%) as a white solid. LC-MS (ESI): mass calcd. for C₂₀H₁₉FN₆O 378.16 m/z, found 379.1 [M+H]⁺. ¹H NMR (400 MHz, CDCl₃): δ 11.50 (br s, 1H), 8.38 (d, J=2.8 Hz, 1H), 7.53-7.47 (m, 2H), 7.32 (dt, J=3.2, 8.4 Hz, 1H), 6.94 (s, 1H), 4.69-4.56 (m, 2H), 2.65 (s, 3H), 2.36-2.25 (m, 1H), 2.09-2.01 (m, 1H), 1.71 (d, J=6.8 Hz, 3H), 1.52 (d, J=6.4 Hz, 3H).

### Example 210: 2-(4-Fluorophenyl)-3-(1H-pyrazolo[3,4-b]pyridin-4-yl)spiro[5,7-dihydropyrazolo[5,1-b][1,3]oxazine-6,3′-oxetane]

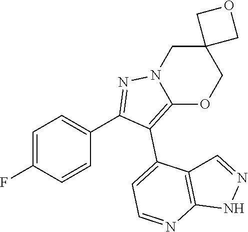 [SMILES: Fc1ccc(-c2nn3c(c2-c2ccnc4[nH]ncc24)OCC2(COC2)C3)cc1]

The title compound was prepared in a manner analogous to Example 1, Steps A-B, except using 4-(4,4,5,5-tetramethyl-1,3,2-dioxaborolan-2-yl)-1-((2-(trimethylsilyl)ethoxy)methyl)-1H-pyrazolo[3,4-b]pyridine (Intermediate 23) instead of 4-(4,4,5,5-tetramethyl-1,3,2-dioxaborolan-2-yl)-1-((2-(trimethylsilyl)ethoxy)methyl)-1H-pyrrolo[2,3-b]pyridine (Intermediate 21) and 3′-bromo-2′-(4-fluorophenyl)-5′H,7′H-spiro[oxetane-3,6′-pyrazolo[5,1-b][1,3]oxazine](Intermediate 126) instead of 3-bromo-2-(5-fluoropyridin-2-yl)-6,7-dihydro-4H-pyrazolo[5,1-c][1,4]oxazine (Intermediate 37) in Step A. MS (ESI): mass calcd. for C₂₀H₁₆FN₅O₂, 377.1; m/z found, 378.1 [M+H]⁺. ¹H NMR (500 MHz, CDCl₃): δ 11.91 (s, 1H), 8.48 (d, J=4.9 Hz, 1H), 7.62 (s, 1H), 7.43-7.34 (m, 2H), 7.02-6.90 (m, 3H), 4.74 (d, J=6.9 Hz, 2H), 4.69 (d, J=6.8 Hz, 2H), 4.60 (s, 2H), 4.53 (s, 2H).

### Example 211: 2′-(4-Fluorophenyl)-3′-(6-methyl-1H-pyrazolo[3,4-b]pyridin-4-yl)-5′H,7′H-spiro[oxetane-3,6′-pyrazolo[5,1-b][1,3]oxazine]

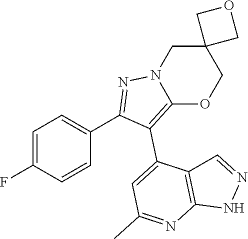 [SMILES: Cc1cc(-c2c(-c3ccc(F)cc3)nn3c2OCC2(COC2)C3)c2cn[nH]c2n1]

The title compound was prepared in a manner analogous to Example 1, Steps A-B, except using 6-methyl-4-(4,4,5,5-tetramethyl-1,3,2-dioxaborolan-2-yl)-1-((2-(trimethylsilyl)ethoxy)methyl)-1H-pyrazolo[3,4-b]pyridine (Intermediate 26) instead of 4-(4,4,5,5-tetramethyl-1,3,2-dioxaborolan-2-yl)-1-((2-(trimethylsilyl)ethoxy)methyl)-1H-pyrrolo[2,3-b]pyridine (Intermediate 21) and 3′-bromo-2′-(4-fluorophenyl)-5′H,7′H-spiro[oxetane-3,6′-pyrazolo[5,1-b][1,3]oxazine](Intermediate 126) instead of 3-bromo-2-(5-fluoropyridin-2-yl)-6,7-dihydro-4H-pyrazolo[5,1-c][1,4]oxazine (Intermediate 37) in Step A. MS (ESI): mass calcd. for C₂₁H₁₈FN₅O₂, 391.1; m/z found, 392.1 [M+H]⁺. ¹H NMR (500 MHz, CDCl₃): δ 11.99 (s, 1H), 7.48 (s, 1H), 7.41-7.36 (m, 2H), 6.99-6.93 (m, 2H), 6.89 (s, 1H), 4.73 (d, J=6.8 Hz, 2H), 4.69 (d, J=6.8 Hz, 2H), 4.59 (s, 2H), 4.52 (s, 2H), 2.68 (s, 3H).

### Example 212: 2-(4-Fluorophenyl)-6,6-dimethyl-3-(1H-pyrazolo[3,4-b]pyridin-4-yl)-6,7-dihydro-5H-pyrazolo[5,1-b][1,3]oxazine

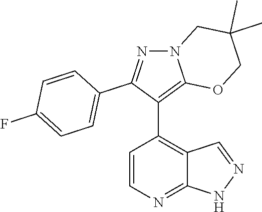 [SMILES: CC1(C)COc2c(-c3ccnc4[nH]ncc34)c(-c3ccc(F)cc3)nn2C1]

The title compound was prepared in a manner analogous to Example 1, Steps A-B, except using 3-bromo-2-(4-fluorophenyl)-6,6-dimethyl-6,7-dihydro-5H-pyrazolo[5,1-b][1,3]oxazine (Intermediate 127) instead of 3-bromo-2-(5-fluoropyridin-2-yl)-6,7-dihydro-4H-pyrazolo[5,1-c][1,4]oxazine (Intermediate 37) and 4-(4,4,5,5-tetramethyl-1,3,2-dioxaborolan-2-yl)-1-((2-(trimethylsilyl)ethoxy)methyl)-1H-pyrazolo[3,4-b]pyridine (Intermediate 23) instead of 4-(4,4,5,5-tetramethyl-1,3,2-dioxaborolan-2-yl)-1-((2-(trimethylsilyl)ethoxy)methyl)-1H-pyrrolo[2,3-b]pyridine (Intermediate 21) in Step A. MS (ESI): mass calcd. for C₂₀H₁₈FN₅O, 363.4; m/z found, 364.2 [M+H]⁺. ¹H NMR (500 MHz, DMSO-d₆) δ 13.49 (s, 1H), 8.38 (d, J=4.8 Hz, 1H), 7.53 (s, 1H), 7.38-7.30 (m, 2H), 7.17-7.08 (m, 2H), 6.89 (d, J=4.8 Hz, 1H), 4.11 (s, 2H), 3.99 (s, 2H), 1.14 (s, 6H).

### Example 213: 2-(4-Fluorophenyl)-6,6-dimethyl-3-(6-methyl-1H-pyrazolo[3,4-b]pyridin-4-yl)-6,7-dihydro-5H-pyrazolo[5,1-b][1,3]oxazine

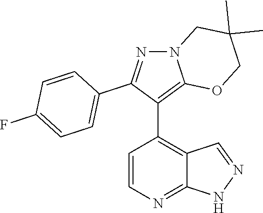 [SMILES: CC1(C)COc2c(-c3ccnc4[nH]ncc34)c(-c3ccc(F)cc3)nn2C1]

The title compound was prepared in a manner analogous to Example 1, except using 3-bromo-2-(4-fluorophenyl)-6,6-dimethyl-6,7-dihydro-5H-pyrazolo[5,1-b][1,3]oxazine (Intermediate 127) instead of 3-bromo-2-(5-fluoropyridin-2-yl)-6,7-dihydro-4H-pyrazolo[5,1-c][1,4]oxazine (Intermediate 37) and 6-methyl-4-(4,4,5,5-tetramethyl-1,3,2-dioxaborolan-2-yl)-1-((2-(trimethylsilyl)ethoxy)methyl)-1H-pyrazolo[3,4-b]pyridine (Intermediate 26) instead of 4-(4,4,5,5-tetramethyl-1,3,2-dioxaborolan-2-yl)-1-((2-(trimethylsilyl)ethoxy)methyl)-1H-pyrrolo[2,3-b]pyridine (Intermediate 21) in Step A. MS (ESI): mass calcd. for C₂₁H₂₀FN₅O, 377.4; m/z found, 378.2 [M+H]⁺. ¹H NMR (500 MHz, Benzene-d₆) δ 8.18 (s, 1H), 7.77-7.70 (m, 2H), 7.06 (s, 1H), 6.88-6.81 (m, 2H), 3.53 (s, 2H), 3.18 (s, 2H), 2.62 (s, 3H), 0.57 (s, 6H).

### Example 214: 2-(5-Fluoropyridin-2-yl)-6,6-dimethyl-3-(thieno[3,2-b]pyridin-7-yl)-6,7-dihydro-5H-pyrazolo[5,1-b][1,3]oxazine

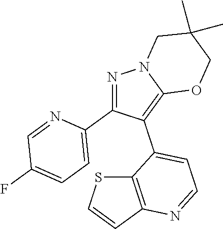 [SMILES: CC1(C)COc2c(-c3ccnc4ccsc34)c(-c3ccc(F)cn3)nn2C1]

The title compound was prepared in a manner analogous to Example 1, Step A, except using 3-bromo-2-(5-fluoro-2-pyridyl)-6,6-dimethyl-5,7-dihydropyrazolo[5,1-b][1,3]oxazine (Intermediate 128) instead of 3-bromo-2-(5-fluoropyridin-2-yl)-6,7-dihydro-4H-pyrazolo[5,1-c][1,4]oxazine (Intermediate 37) and 7-(4,4,5,5-tetramethyl-1,3,2-dioxaborolan-2-yl)thieno[3,2-b]pyridine (Intermediate 29) instead of 4-(4,4,5,5-tetramethyl-1,3,2-dioxaborolan-2-yl)-1-((2-(trimethylsilyl)ethoxy)methyl)-1H-pyrrolo[2,3-b]pyridine (Intermediate 21). MS (ESI): mass calcd. for C₂₀H₁₇FN₄OS, 380.1; m/z found, 381.1 [M+H]⁺. ¹H NMR (400 MHz, CD₃OD) δ 8.77 (d, J=6.1 Hz, 1H), 8.45 (d, J=5.7 Hz, 1H), 8.13 (s, 1H), 8.00 (dd, J=8.8, 4.6 Hz, 1H), 7.77 (d, J=6.1 Hz, 1H), 7.74-7.59 (m, 2H), 4.20 (s, 2H), 4.10 (s, 2H), 1.26 (s, 6H).

### Example 215: N-(4-(2-(4-Fluorophenyl)-5,6,7,8-tetrahydropyrazolo[5,1-b][1,3]oxazepin-3-yl)pyridin-2-yl)acetamide

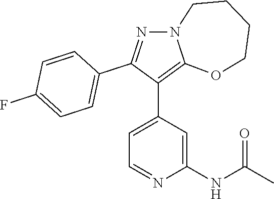 [SMILES: CC(=O)Nc1cc(-c2c(-c3ccc(F)cc3)nn3c2OCCCC3)ccn1]

The title compound was prepared in a manner analogous to Example 1, Step A, except using 3-bromo-2-(4-fluorophenyl)-5,6,7,8-tetrahydropyrazolo[5,1-b][1,3]oxazepane (Intermediate 129) instead 3-bromo-2-(5-fluoropyridin-2-yl)-6,7-dihydro-4H-pyrazolo[5,1-c][1,4]oxazine (Intermediate 37) and N-(4-(4,4,5,5-tetramethyl-1,3,2-dioxaborolan-2-yl)pyridin-2-yl)acetamide instead of 4-(4,4,5,5-tetramethyl-1,3,2-dioxaborolan-2-yl)-1-((2-(trimethylsilyl)ethoxy)methyl)-1H-pyrrolo[2,3-b]pyridine (Intermediate 21) in Step A. MS (ESI): mass calcd. for C₂₀H₁₉FN₄O₂, 366.4; m/z found, 367.0 [M+H]⁺. ¹H NMR (500 MHz, CD₃OD) δ 8.10 (dd, J=5.3, 0.8 Hz, 1H), 8.08 (s, 1H), 7.44-7.37 (m, 2H), 7.13-7.06 (m, 2H), 6.87 (dd, J=5.3, 1.6 Hz, 1H), 4.33-4.25 (m, 2H), 4.24-4.18 (m, 2H), 2.17-2.14 (m, 2H), 2.13 (s, 3H), 1.99-1.91 (m, 2H).

### Example 216: 2-(4-Fluorophenyl)-3-(1H-pyrazolo[3,4-b]pyridin-4-yl)-5,6,7,8-tetrahydropyrazolo[5,1-b][1,3]oxazepane

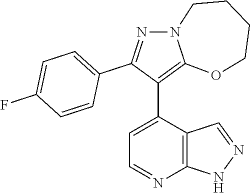 [SMILES: Fc1ccc(-c2nn3c(c2-c2ccnc4[nH]ncc24)OCCCC3)cc1]

The title compound was prepared in a manner analogous to Example 1, Steps A-B, except using 3-bromo-2-(4-fluorophenyl)-5,6,7,8-tetrahydropyrazolo[5,1-b][1,3]oxazepane (Intermediate 129) instead of 3-bromo-2-(5-fluoropyridin-2-yl)-6,7-dihydro-4H-pyrazolo[5,1-c][1,4]oxazine (Intermediate 37) and 4-(4,4,5,5-tetramethyl-1,3,2-dioxaborolan-2-yl)-1-((2-(trimethylsilyl)ethoxy)methyl)-1H-pyrazolo[3,4-b]pyridine (Intermediate 23) instead of 4-(4,4,5,5-tetramethyl-1,3,2-dioxaborolan-2-yl)-1-((2-(trimethylsilyl)ethoxy)methyl)-1H-pyrrolo[2,3-b]pyridine (Intermediate 21) in Step A. MS (ESI): mass calcd. for C₁₉H₁₆FN₅O, 349.4; m/z found, 350.2 [M+H]⁺. ¹H NMR (500 MHz, Benzene-d₆) δ 13.76 (s, 1H), 8.41-8.32 (m, 1H), 8.12 (s, 1H), 7.57-7.49 (m, 2H), 6.95-6.89 (m, 1H), 6.77-6.67 (m, 2H), 3.94-3.83 (m, 2H), 3.43-3.33 (m, 2H), 1.27-1.07 (m, 4H).

### Example 217: 2-(4-Fluorophenyl)-3-(6-methyl-1H-pyrazolo[3,4-b]pyridin-4-yl)-5,6,7,8-tetrahydropyrazolo[5,1-b][1,3]oxazepane

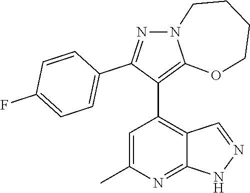 [SMILES: Cc1cc(-c2c(-c3ccc(F)cc3)nn3c2OCCCC3)c2cn[nH]c2n1]

The title compound was prepared in a manner analogous to Example 1, Steps A-B, except using 3-bromo-2-(4-fluorophenyl)-5,6,7,8-tetrahydropyrazolo[5,1-b][1,3]oxazepane (Intermediate 129) instead of 3-bromo-2-(5-fluoropyridin-2-yl)-6,7-dihydro-4H-pyrazolo[5,1-c][1,4]oxazine (Intermediate 37) and 6-methyl-4-(4,4,5,5-tetramethyl-1,3,2-dioxaborolan-2-yl)-1-((2-(trimethylsilyl)ethoxy)methyl)-1H-pyrazolo[3,4-b]pyridine (Intermediate 26) instead of 4-(4,4,5,5-tetramethyl-1,3,2-dioxaborolan-2-yl)-1-((2-(trimethylsilyl)ethoxy)methyl)-1H-pyrrolo[2,3-b]pyridine (Intermediate 21) in Step A. MS (ESI): mass calcd. for C₂₀H₁₈FN₅O, 363.4; m/z found, 364.2 [M+H]⁺. ¹H NMR (500 MHz, CD₃OD) δ 7.43 (s, 1H), 7.38-7.31 (m, 2H), 7.04-6.98 (m, 2H), 6.92 (s, 1H), 4.40-4.32 (m, 2H), 4.18-4.11 (m, 2H), 2.57 (s, 3H), 2.17-2.09 (m, 2H), 2.03-1.94 (m, 2H).

### Example 218: 2-(5-Fluoro-2-pyridyl)-3-(1H-pyrazolo[3,4-b]pyridin-4-yl)-5,6,7,8-tetrahydropyrazolo[5,1-b][1,3]oxazepane

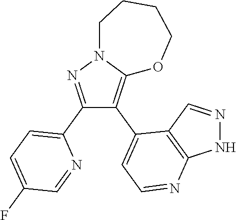 [SMILES: Fc1ccc(-c2nn3c(c2-c2ccnc4[nH]ncc24)OCCCC3)nc1]

The title compound was prepared in a manner analogous to Example 1, Steps A-B, except using 3-bromo-2-(5-fluoropyridin-2-yl)-5,6,7,8-tetrahydropyrazolo[5,1-b][1,3]oxazepane (Intermediate 130) instead of 3-bromo-2-(5-fluoropyridin-2-yl)-6,7-dihydro-4H-pyrazolo[5,1-c][1,4]oxazine (Intermediate 37) and 4-(4,4,5,5-tetramethyl-1,3,2-dioxaborolan-2-yl)-1-((2-(trimethylsilyl)ethoxy)methyl)-1H-pyrazolo[3,4-b]pyridine (Intermediate 23) instead of 4-(4,4,5,5-tetramethyl-1,3,2-dioxaborolan-2-yl)-1-((2-(trimethylsilyl)ethoxy)methyl)-1H-pyrrolo[2,3-b]pyridine (Intermediate 21) in Step A. LC-MS (ESI): mass calcd. for C₁₈H₁₅FN₆O 350.13 m/z, found 351.1 [M+H]⁺. ¹H NMR (400 MHz, CDCl₃): δ 11.40 (br s, 1H), 8.50 (d, J=4.8 Hz, 1H), 8.37 (d, J=2.8 Hz, 1H), 7.62 (s, 1H), 7.42 (dd, J=4.4, 8.8 Hz, 1H), 7.30 (dt, J=2.8, 8.4 Hz, 1H), 7.04 (d, J=5.2 Hz, 1H), 4.49-4.39 (m, 2H), 4.17-4.03 (m, 2H), 2.18-2.11 (m, 2H), 2.04-1.98 (m, 2H).

### Example 219: 2-(5-Fluoro-2-pyridyl)-3-(6-methyl-1H-pyrazolo[3,4-b]pyridin-4-yl)-5,6,7,8-tetrahydropyrazolo[5,1-b][1,3]oxazepane

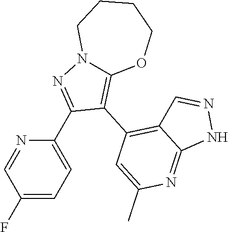 [SMILES: Cc1cc(-c2c(-c3ccc(F)cn3)nn3c2OCCCC3)c2cn[nH]c2n1]

The title compound was prepared in a manner analogous to Example 1, Steps A-B, except using 3-bromo-2-(5-fluoropyridin-2-yl)-5,6,7,8-tetrahydropyrazolo[5,1-b][1,3]oxazepane (Intermediate 130) instead of 3-bromo-2-(5-fluoropyridin-2-yl)-6,7-dihydro-4H-pyrazolo[5,1-c][1,4]oxazine (Intermediate 37) and 6-methyl-4-(4,4,5,5-tetramethyl-1,3,2-dioxaborolan-2-yl)-1-((2-(trimethylsilyl)ethoxy)methyl)-1H-pyrazolo[3,4-b]pyridine (Intermediate 26) instead of 4-(4,4,5,5-tetramethyl-1,3,2-dioxaborolan-2-yl)-1-((2-(trimethylsilyl)ethoxy)methyl)-1H-pyrrolo[2,3-b]pyridine (Intermediate 21) in Step A. LC-MS (ESI): mass calcd. for C₁₉H₁₇FN₆O 364.15 m/z, found 365.1 [M+H]⁺. ¹H NMR (400 MHz, CDCl₃): δ 8.38 (d, J=2.8 Hz, 1H), 7.50 (s, 1H), 7.45 (dd, J=2.8, 8.8 Hz, 1H), 7.32 (dt, J=2.8, 8.4 Hz, 1H), 6.97 (s, 1H), 4.49-4.42 (m, 2H), 4.16-4.09 (m, 2H), 2.69 (s, 3H), 2.22-2.11 (m, 2H), 2.07-1.98 (m, 2H).

### Example 220: 7-[2-(5-Fluoro-2-pyridyl)-5,6-dihydro-4H-pyrrolo[1,2-b]pyrazol-3-yl]-1H-pyrazolo[4,3-b]pyridine

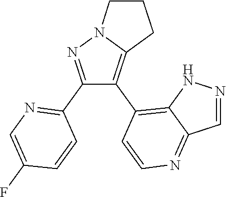 [SMILES: Fc1ccc(-c2nn3c(c2-c2ccnc4cn[nH]c24)CCC3)nc1]

The title compound was prepared in a manner analogous to 4-[6,6-difluoro-2-(4-fluorophenyl)-5,7-dihydro-4H-pyrazolo[1,5-a]pyridin-3-yl]-6-methyl-1H-pyrazolo[3,4-d]pyrimidine (Example 279), Steps A-B, except using 3-bromo-2-(4-fluorophenyl)-5,6-dihydro-4H-pyrrolo[1,2-b]pyrazole (Intermediate 38) instead of Intermediate 52 and 7-chloro-1-((2-(trimethylsilyl)ethoxy)methyl)-1H-pyrazolo[4,3-b]pyridine (Intermediate 191) instead of Intermediate 185 in Step A. MS (ESI): mass calcd. for C₁₇H₁₃FN₆ 320.1; m/z found 321.1 [M+H]⁺. ¹H NMR (400 MHz, DMSO-d₆): δ 12.95 (s, 1H), 8.41 (d, J=4.5 Hz, 1H), 8.25 (s, 1H), 8.16 (d, J=2.8 Hz, 1H), 7.96-7.88 (m, 1H), 7.78-7.69 (m, 1H), 7.12 (d, J=4.5 Hz, 1H), 4.26 (t, J=7.3 Hz, 2H), 2.94-2.81 (m, 2H), 2.63 (quin, J=7.2 Hz, 2H).

### Example 221: 5-Fluoro-4-[2-(5-fluoro-2-pyridyl)-5,6-dihydro-4H-pyrrolo[1,2-b]pyrazol-3-yl]-1H-pyrazolo[3,4-b]pyridine

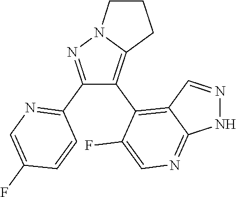 [SMILES: Fc1ccc(-c2nn3c(c2-c2c(F)cnc4[nH]ncc24)CCC3)nc1]

Step A. 2-(2-(5-Fluoropyridin-2-yl)-5,6-dihydro-4H-pyrrolo[1,2-b]pyrazol-3-yl)-2-hydroxy-4,4,5,5-tetramethyl-1,3,2-dioxaborolan-2-uide. n-BuLi (2.1 mL, 1 M in THF and hexane, 2.1 mmol) was added dropwise to a −78° C. mixture consisting of 3-bromo-2-(5-fluoropyridin-2-yl)-5,6-dihydro-4H-pyrrolo[1,2-b]pyrazole (Intermediate 39, 200 mg, 0.709 mmol) and 2-isopropoxy-4,4,5,5-tetramethyl-1,3,2-dioxaborolane (434 μL, 2.13 mmol). The mixture was stirred at −78° C. for 1 hour and stirred at room-temperature for 2 hours. The mixture was quenched with water (64 μL, 3.6 mmol), stirred at room-temperature for 1 hour and the suspension isolated via filtration. The filter cake was washed ethyl acetate (5 mL×3) before drying under reduced pressure to afford the title compound (400 mg, crude) as a white solid, which was used in the next step without further purification. MS (ESI): mass calcd. for C₁₇H₂₁BFN₃O₂ 329.2; m/z found 280.8 [M-Pin+Na]⁺. ¹H NMR (400 MHz, CDCl₃) δ 8.44 (d, J=2.8 Hz, 1H), 8.03-7.96 (m, 1H), 7.71-7.63 (m, 1H), 4.10 (t, J=7.3 Hz, 2H), 2.96 (d, J=6.8 Hz, 2H), 2.63-2.56 (m, 2H), 1.20 (s, 12H).

Step B. 5-Fluoro-4-(2-(5-fluoropyridin-2-yl)-5,6-dihydro-4H-pyrrolo[1,2-b]pyrazol-3-yl)-1-((2-(trimethylsilyl)ethoxy)methyl)-1H-pyrazolo[3,4-b]pyridine. 2-(2-(5-Fluoropyridin-2-yl)-5, 6-dihydro-4H-pyrrolo[1,2-b]pyrazol-3-yl)-2-hydroxy-4,4,5,5-tetramethyl-1,3,2-dioxaborolan-2-uide (350 mg, 0.595 mmol), 5-fluoro-4-iodo-1-((2-(trimethylsilyl)ethoxy)methyl)-1H-pyrazolo[3,4-b]pyridine (Intermediate 204, 156 mg, 0.397 mmol), and Cs₂CO₃ (388 mg, 1.19 mmol) were added to a 40 mL flask and the resulting mixture dissolved in 2-methyl-2-butanol (10 mL) and H₂O (2 mL). The resultant mixture was sparged with N₂ for 5 minutes and then treated with CataCXium® A Pd G3 (29 mg, 0.040 mmol). The resultant mixture was sparged with N₂ for another 5 minutes and heated at 90° C. for 16 hours. The reaction mixture was cooled to room-temperature. The mixture was filtered, the filter cake was washed with ethyl acetate (5 mL×3). The filtrate was concentrated to dryness under reduced pressure and purified by FCC (SiO₂, petroleum ether:ethyl acetate=1:0 to 1:1) to afford the product (220 mg, 81%) as a yellow solid. MS (ESI) mass calcd. for C₂₃H₂₆F₂N₆OSi 468.2; m/z found 469.2 [M+H]⁺.

Step C. 5-Fluoro-4-(2-(5-fluoropyridin-2-yl)-5,6-dihydro-4H-pyrrolo[1,2-b]pyrazol-3-yl)-1H-pyrazolo[3,4-b]pyridine. TFA (10 mL) was added to 5-fluoro-4-(2-(5-fluoropyridin-2-yl)-5,6-dihydro-4H-pyrrolo[1,2-b]pyrazol-3-yl)-1-((2-(trimethylsilyl)ethoxy)methyl)-1H-pyrazolo[3,4-b]pyridine (220 mg, 0.282 mmol) in a 100 mL round-bottomed flask. The mixture was stirred at room-temperature for 1 hour. The mixture was concentrated to dryness under reduced pressure to give the residue, which was suspended in NH₃/CH₃₀H (2 M, 10 mL) and stirred for 30 mins. The mixture was concentrated to dryness under reduced pressure. The resulting residue was purified by preparative HPLC using a Boston Prime C18 150*30 mm*5 um column (eluent: 25% to 55% (v/v) CH₃CN and H₂O with 0.05% NH₃H₂O) to afford still-impure product. The impure product was further purified by SFC over DAICEL CHIRALCEL OD-H 250 mm×30 mm×5 m (isocratic elution: EtOH (containing 0.1% of 25% aq. NH₃): supercritical CO₂, 30%: 70% to 30%: 70% (v/v)). The pure fractions were collected and the volatiles were removed under reduced pressure to afford the title compound (15.0 mg, 16%) as a white solid. MS (ESI): mass calcd. for C₁₇H₁₂F₂N₆ 338.1; m/z found 339.1 [M+H]⁺. ¹H NMR (400 MHz, DMSO-d₆): δ 13.69 (s, 1H), 8.47 (d, J=2.5 Hz, 1H), 8.22 (d, J=2.5 Hz, 1H), 7.92 (dd, J=4.5, 8.8 Hz, 1H), 7.80-7.74 (m, 1H), 7.72 (s, 1H), 4.28 (t, J=7.2 Hz, 2H), 2.95-2.87 (m, 2H), 2.70-2.58 (m, 2H).

### Example 222: 6-(Difluoromethyl)-4-[2-(5-fluoro-2-pyridyl)-5,6-dihydro-4H-pyrrolo[1,2-b]pyrazol-3-yl]-1H-pyrazolo[3,4-b]pyridine

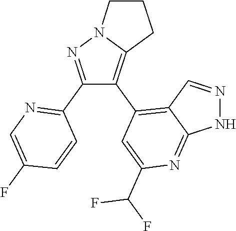 [SMILES: Fc1ccc(-c2nn3c(c2-c2cc(C(F)F)nc4[nH]ncc24)CCC3)nc1]

The title compound was prepared in a manner analogous to Example 1, Steps A-B, except using 3-bromo-2-(5-fluoropyridin-2-yl)-5,6-dihydro-4H-pyrrolo[1,2-b]pyrazole (Intermediate 39) instead of Intermediate 37 and 6-(difluoromethyl)-4-(4,4,5,5-tetramethyl-1,3,2-dioxaborolan-2-yl)-1-((2-(trimethylsilyl)ethoxy)methyl)-1H-pyrazolo[3,4-b]pyridine (Intermediate 221) instead of 4-(4,4,5,5-tetramethyl-1,3,2-dioxaborolan-2-yl)-1-((2-(trimethylsilyl)ethoxy)methyl)-1H-pyrrolo[2,3-b]pyridine (Intermediate 21) in Step A. MS (ESI): mass calcd. for C₁₈H₁₃F₃N₆ 370.1; m/z found 371.0 [M+H]⁺. ¹H NMR (400 MHz, DMSO-d₆): δ 13.81 (s, 1H), 8.24 (d, J=2.9 Hz, 1H), 7.90 (dd, J=4.6, 8.8 Hz, 1H), 7.84-7.77 (m, 1H), 7.54 (s, 1H), 7.29 (s, 1H), 7.02 (t, J=56.0 Hz, 1H), 4.27 (t, J=7.3 Hz, 2H), 3.03 (t, J=7.3 Hz, 2H), 2.69-2.58 (m, 2H).

### Example 223: 1-Ethyl-5-(2-(5-fluoropyridin-2-yl)-5,6-dihydro-4H-pyrrolo[1,2-b]pyrazol-3-yl)-1H-pyrazolo[3,4-b]pyridine

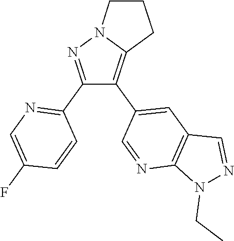 [SMILES: CCn1ncc2cc(-c3c(-c4ccc(F)cn4)nn4c3CCC4)cnc21]

3-Bromo-2-(5-fluoropyridin-2-yl)-5,6-dihydro-4H-pyrrolo[1,2-b]pyrazole (Intermediate 39, 50 mg, 0.18 mmol), 1-ethyl-5-(4,4,5,5-tetramethyl-1,3,2-dioxaborolan-2-yl)-1H-pyrazolo[3,4-b]pyridine (Intermediate 31, 53 mg, 0.19 mmol), CataCXium® A Pd G3 (13 mg, 0.018 mmol), and Cs₂CO₃ (173 mg, 0.532 mmol) were suspended in a mixture of 2-methyl-2-butanol (0.82 mL) and water (0.13 mL). The vial was flushed with nitrogen, sealed, and placed in a preheated heating block at 85-90° C. for 5.5 hours. The reaction mixture was cooled, diluted with water, and extracted with ethyl acetate. The combined organics were dried with MgSO₄, filtered, and concentrated under reduced pressure. Purification (Basic AQQU Prep with ACN/NH₄OH 0-100% over 25 min) afforded the title compound (52.9 mg, 0.152 mmol, 86%). MS (ESI): mass calcd. for C₁₉H₁₇FN₆ 348.1; m/z found 349.2 [M+H]⁺. ¹H NMR (600 MHz, DMSO-d₆): δ 8.46 (d, J=2.1 Hz, 1H), 8.39-8.35 (m, 1H), 8.11 (d, J=2.0 Hz, 1H), 8.10 (s, 1H), 7.90-7.85 (m, 1H), 7.76 (td, J=8.8, 3.0 Hz, 1H), 4.49 (q, J=7.2 Hz, 2H), 4.23 (t, J=7.3 Hz, 2H), 3.02 (t, J=7.3 Hz, 2H), 2.66-2.62 (m, 2H), 1.45 (t, J=7.2 Hz, 3H).

### Example 224: 3-Chloro-5-fluoro-4-(2-(5-fluoropyridin-2-yl)-5,6-dihydro-4H-pyrrolo[1,2-b]pyrazol-3-yl)-6-methyl-1H-pyrazolo[3,4-b]pyridine

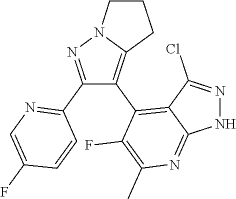 [SMILES: Cc1nc2[nH]nc(Cl)c2c(-c2c(-c3ccc(F)cn3)nn3c2CCC3)c1F]

To a solution of 5-fluoro-4-(2-(5-fluoropyridin-2-yl)-5,6-dihydro-4H-pyrrolo[1,2-b]pyrazol-3-yl)-6-methyl-1H-pyrazolo[3,4-b]pyridine (Example 9) in DMF (0.84 mL) was added sodium hydride (60% dispersion in mineral oil, 4 mg, 0.1 mmol). The reaction mixture was stirred for 10 minutes before N-chlorosuccinimide (12.9 mg, 0.097 mmol) was added. After 2.25 hours the reaction mixture was diluted with water, extracted with ethyl acetate (×3), and concentrated under reduced pressure. Purification (ACCQ-prep. HPLC (0.05% TFA in H₂O and 0.05% TFA in CH₃CN) followed by lyophilization of the pure fractions afforded the title compound (16.3 mg, 0.0421 mmol, 50%). MS (ESI): mass calcd. for C₁₈H₁₃ClF₂N₆ 386.1; m/z found 387.1 [M+H]⁺. ¹H NMR (400 MHz, CD₃OD): δ 8.06 (dd, J=2.9, 0.7 Hz, 1H), 7.90-7.80 (m, 1H), 7.56 (td, J=8.6, 2.9 Hz, 1H), 4.33 (td, J=7.7, 7.1, 2.0 Hz, 2H), 2.93 (dd, J=7.9, 6.3 Hz, 2H), 2.81-2.68 (m, 2H), 2.60 (d, J=3.6 Hz, 3H). N—H proton not observed.

### Example 225: 4-[2-(4-Fluorophenyl)-5,6-dihydro-4H-pyrrolo[1,2-b]pyrazol-3-yl]-6-methyl-1H-pyrazolo[3,4-d]pyrimidine

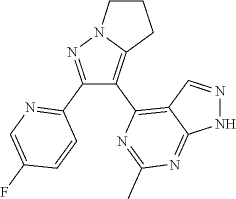 [SMILES: Cc1nc(-c2c(-c3ccc(F)cn3)nn3c2CCC3)c2cn[nH]c2n1]

The title compound was prepared in a manner analogous to 4-[6,6-difluoro-2-(4-fluorophenyl)-5,7-dihydro-4H-pyrazolo[1,5-a]pyridin-3-yl]-6-methyl-1H-pyrazolo[3,4-d]pyrimidine (Example 279), Steps A-B except using 3-bromo-2-(4-fluorophenyl)-5,6-dihydro-4H-pyrrolo[1,2-b]pyrazole (Intermediate 38) instead of 3-bromo-6,6-difluoro-2-(4-fluorophenyl)-4,5,6,7-tetrahydropyrazolo[1,5-a]pyridine (Intermediate 52) in Step A. MS (ESI): mass calcd. for C₁₈H₁₅FN₆ 334.1; m/z found 335.0 [M+H]⁺. ¹H NMR (400 MHz, DMSO-d₆): δ 7.49-7.44 (m, 2H), 7.19-7.14 (m, 2H), 7.14 (s, 1H), 4.23 (t, J=7.3 Hz, 2H), 3.11 (t, J=7.3 Hz, 2H), 2.67-2.59 (m, 5H). N—H proton not observed.

### Example 226: 5-[(5S)-5-Fluoro-2-(4-fluorophenyl)-5,6-dihydro-4H-pyrrolo[1,2-b]pyrazol-3-yl]pyrazolo[1,5-a]pyridine

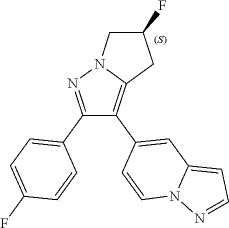

The title compound was prepared in a manner analogous to (5a*R,6a*S)-6,6-difluoro-2-(4-fluorophenyl)-3-(pyrazolo[1,5-a]pyridin-4-yl)-5,5a,6,6a-tetrahydro-4H-cyclopropa[e]pyrazolo[1,5-a]pyridine (Example 354), except using (S)-3-bromo-5-fluoro-2-(4-fluorophenyl)-5,6-dihydro-4H-pyrrolo[1,2-b]pyrazole (Intermediate 41) instead of racemic 3-bromo-6,6-difluoro-2-(4-fluorophenyl)-5, 5a,6,6a-tetrahydro-4H-cyclopropa[e]pyrazolo[1,5-a]pyridine (Intermediate 181), and no chiral SFC was performed. MS (ESI): mass calcd. for C₁₉H₁₄F₂N₄ 336.1; m/z found 337.1 [M+H]⁺. ¹H NMR (400 MHz, Chloroform-d): δ 8.36 (dt, J=7.2, 1.0 Hz, 1H), 7.94 (d, J=2.3 Hz, 1H), 7.54-7.42 (m, 2H), 7.37 (dd, J=1.9, 1.0 Hz, 1H), 7.09-6.98 (m, 2H), 6.57-6.51 (m, 1H), 6.45 (dd, J=2.3, 0.9 Hz, 1H), 5.98-5.70 (m, 1H), 4.58-4.42 (m, 2H), 3.57-3.20 (m, 2H).

### Example 227: 5-Fluoro-4-[(5S)-5-fluoro-2-(4-fluorophenyl)-5,6-dihydro-4H-pyrrolo[1,2-b]pyrazol-3-yl]-1H-pyrazolo[3,4-b]pyridine

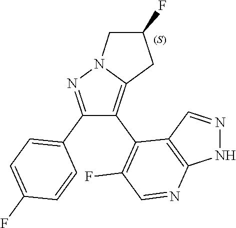

The title compound was prepared in a manner analogous to 4-(6,6-difluoro-2-(4-fluorophenyl)-4,5,6,7-tetrahydropyrazolo[1,5-a]pyridin-3-yl)-5-fluoro-1H-pyrazolo[3,4-b]pyridine (Example 280) Steps A-B, except using (S)-3-bromo-5-fluoro-2-(4-fluorophenyl)-5,6-dihydro-4H-pyrrolo[1,2-b]pyrazole (Intermediate 41) instead of 3-bromo-6,6-difluoro-2-(4-fluorophenyl)-4,5,6,7-tetrahydropyrazolo[1,5-a]pyridine (Intermediate 52) in Step A. MS (ESI): mass calcd. for C₁₈H₁₂F₃N₅ 355.1; m/z found 356.1 [M+H]⁺. ¹H NMR (400 MHz, Chloroform-d): δ 10.79 (s, 1H), 8.51 (d, J=2.8 Hz, 1H), 7.43-7.35 (m, 2H), 7.33 (s, 1H), 6.98-6.90 (m, 2H), 5.99-5.64 (m, 1H), 4.67-4.40 (m, 2H), 3.54-3.11 (m, 2H).

### Example 228: 4-[(5R)-5-Fluoro-2-(4-fluorophenyl)-5,6-dihydro-4H-pyrrolo[1,2-b]pyrazol-3-yl]-1H-pyrazolo[3,4-b]pyridine

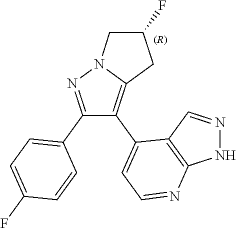

The title compound was prepared in a manner analogous to Example 1, Steps A-B, except using (R)-3-bromo-5-fluoro-2-(4-fluorophenyl)-5,6-dihydro-4H-pyrrolo[1,2-b]pyrazole (Intermediate 150) instead of 3-bromo-2-(5-fluoropyridin-2-yl)-6,7-dihydro-4H-pyrazolo[5,1-c][1,4]oxazine (Intermediate 37); 4-(4,4,5,5-tetramethyl-1,3,2-dioxaborolan-2-yl)-1-((2-(trimethylsilyl)ethoxy)methyl)-1H-pyrazolo[3,4-b]pyridine (Intermediate 23) instead of 4-(4,4,5,5-tetramethyl-1,3,2-dioxaborolan-2-yl)-1-((2-(trimethylsilyl)ethoxy)methyl)-1H-pyrrolo[2,3-b]pyridine (Intermediate 21); and Pd(dppf)Cl₂ instead of CataCXium® A Pd G3 in Step A. MS (ESI): mass calcd. for C₁₈H₁₃F₂N₅, 337.1; m/z found 338.0 [M+H]⁺. ¹H NMR (400 MHz, DMSO-d₆): δ 13.60 (br s, 1H), 8.46 (d, J=4.5 Hz, 1H), 7.42 (s, 1H), 7.39-7.32 (m, 2H), 7.17-7.10 (m, 2H), 7.04 (d, J=4.8 Hz, 1H), 6.04-5.83 (m, 1H), 4.69-4.38 (m, 2H), 3.69-3.49 (m, 1H), 3.26-3.10 (m, 1H).

### Example 229: N-[4-[(5S)-5-Fluoro-2-(4-fluorophenyl)-5,6-dihydro-4H-pyrrolo[1,2-b]pyrazol-3-yl]-2-pyridyl]acetamide

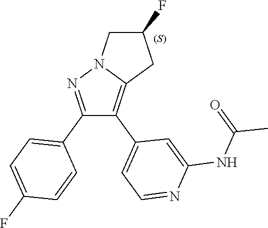

The title compound was prepared in a manner analogous to (5a*R,6a*S)-6,6-difluoro-2-(4-fluorophenyl)-3-(pyrazolo[1,5-a]pyridin-4-yl)-5,5a,6,6a-tetrahydro-4H-cyclopropa[e]pyrazolo[1,5-a]pyridine (Example 354), except using (S)-3-bromo-5-fluoro-2-(4-fluorophenyl)-5,6-dihydro-4H-pyrrolo[1,2-b]pyrazole (Intermediate 41) instead of racemic 3-bromo-6,6-difluoro-2-(4-fluorophenyl)-5,5a,6,6a-tetrahydro-4H-cyclopropa[e]pyrazolo[1,5-a]pyridine (Intermediate 181); N-(4-(4,4,5,5-tetramethyl-1,3,2-dioxaborolan-2-yl)pyridin-2-yl)acetamide was used instead of pyrazolo[1,5-a]pyridin-5-ylboronic acid, and no chiral SFC was performed. MS (ESI): mass calcd. for C₁₉H₁₆F₂N₄O 354.1; m/z found 355.1 [M+H]⁺. ¹H NMR (500 MHz, Chloroform-d): δ 8.28-7.87 (m, 3H), 7.53-7.41 (m, 2H), 7.10-6.98 (m, 2H), 6.75 (dd, J=5.3, 1.6 Hz, 1H), 5.91-5.74 (m, 1H), 4.51 (d, J=3.0 Hz, 1H), 4.47-4.43 (m, 1H), 3.62-3.34 (m, 2H), 2.21 (s, 3H).

### Example 230: 4-[5,5-Difluoro-2-(4-fluorophenyl)-4,6-dihydropyrrolo[1,2-b]pyrazol-3-yl]-1H-pyrazolo[3,4-b]pyridine

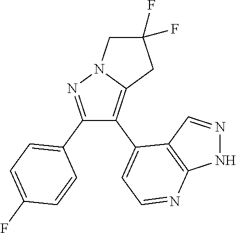 [SMILES: Fc1ccc(-c2nn3c(c2-c2ccnc4[nH]ncc24)CC(F)(F)C3)cc1]

The title compound was prepared in a manner analogous to Example 1, Steps A-B, except using 3-bromo-5,5-difluoro-2-(4-fluorophenyl)-5,6-dihydro-4H-pyrrolo[1,2-b]pyrazole (Intermediate 155) instead of 3-bromo-2-(5-fluoropyridin-2-yl)-6,7-dihydro-4H-pyrazolo[5,1-c][1,4]oxazine (Intermediate 37); 4-(4,4,5,5-tetramethyl-1,3,2-dioxaborolan-2-yl)-1-((2-(trimethylsilyl)ethoxy)methyl)-1H-pyrazolo[3,4-b]pyridine (Intermediate 23) instead of 4-(4,4,5,5-tetramethyl-1,3,2-dioxaborolan-2-yl)-1-((2-(trimethylsilyl)ethoxy)methyl)-1H-pyrrolo[2,3-b]pyridine (Intermediate 21), and Pd(dppf)Cl₂ instead of CataCXium A Pd G3 in Step A. MS (ESI): mass calcd. for C₁₈H₁₂F₃N₅ 355.1; m/z found 356.0 [M+H]⁺. ¹H NMR (400 MHz, DMSO-d₆): δ 13.63 (s, 1H), 8.47 (d, J=4.8 Hz, 1H), 7.44 (s, 1H), 7.39-7.32 (m, 2H), 7.18-7.11 (m, 2H), 7.08 (d, J=4.8 Hz, 1H), 4.86 (t, J=13.0 Hz, 2H), 3.81 (t, J=14.1 Hz, 2H).

### Example 231: 4-[2-(4-Fluorophenyl)-5,5-dimethyl-4,6-dihydropyrrolo[1,2-b]pyrazol-3-yl]-1H-pyrazolo[3,4-b]pyridine

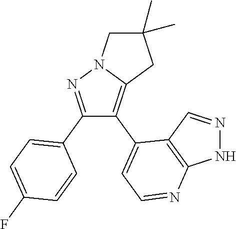 [SMILES: CC1(C)Cc2c(-c3ccnc4[nH]ncc34)c(-c3ccc(F)cc3)nn2C1]

The title compound was prepared in a manner analogous to Example 1, Steps A-B, except using 3-bromo-2-(4-fluorophenyl)-5,5-dimethyl-5,6-dihydro-4H-pyrrolo[1,2-b]pyrazole (Intermediate 151) instead of 3-bromo-2-(5-fluoropyridin-2-yl)-6,7-dihydro-4H-pyrazolo[5,1-c][1,4]oxazine (Intermediate 37); 4-(4,4,5,5-tetramethyl-1,3,2-dioxaborolan-2-yl)-1-((2-(trimethylsilyl)ethoxy)methyl)-1H-pyrazolo[3,4-b]pyridine (Intermediate 23) instead of 4-(4,4,5,5-tetramethyl-1,3,2-dioxaborolan-2-yl)-1-((2-(trimethylsilyl)ethoxy)methyl)-1H-pyrrolo[2,3-b]pyridine (Intermediate 21); and Pd(dppf)₂Cl₂ instead of CataCXium® A Pd G3 in Step A. MS (ESI): mass calcd. for C₂₀H₁₈FN₅ 347.2; m/z found 348.2 [M+H]⁺. ¹H NMR (400 MHz, DMSO-d₆): δ 13.58 (s, 1H), 8.42 (d, J=4.6 Hz, 1H), 7.45 (s, 1H), 7.40-7.26 (m, 2H), 7.12 (t, J=8.9 Hz, 2H), 6.97 (d, J=4.8 Hz, 1H), 4.02 (s, 2H), 2.85 (s, 2H), 1.29 (s, 6H).

### Example 232: 4-[2-(4-Fluorophenyl)-5,5-dimethyl-4,6-dihydropyrrolo[1,2-b]pyrazol-3-yl]-6-methyl-1H-pyrazolo[3,4-b]pyridine

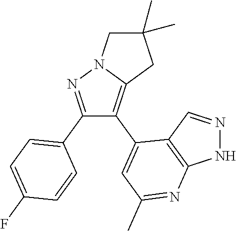 [SMILES: Cc1cc(-c2c(-c3ccc(F)cc3)nn3c2CC(C)(C)C3)c2cn[nH]c2n1]

The title compound was prepared in a manner analogous to Example 1, Steps A-B, except using 3-bromo-2-(4-fluorophenyl)-5,5-dimethyl-5,6-dihydro-4H-pyrrolo[1,2-b]pyrazole (Intermediate 151) instead of 3-bromo-2-(5-fluoropyridin-2-yl)-6,7-dihydro-4H-pyrazolo[5,1-c][1,4]oxazine (Intermediate 37); 6-methyl-4-(4,4,5,5-tetramethyl-1,3,2-dioxaborolan-2-yl)-1-((2-(trimethylsilyl)ethoxy)methyl)-1H-pyrazolo[3,4-b]pyridine (Intermediate 26) instead of 4-(4,4,5,5-tetramethyl-1,3,2-dioxaborolan-2-yl)-1-((2-(trimethylsilyl)ethoxy)methyl)-1H-pyrrolo[2,3-b]pyridine (Intermediate 21); and Pd(dppf)₂Cl₂ instead of CataCXium® A Pd G3 in Step A. MS (ESI): mass calcd. for C₂₁H₂₀FN₅ 361.2; m/z found 362.2 [M+H]⁺. ¹H NMR (400 MHz, CDCl₃): δ 12.59 (br s, 1H), 7.47 (s, 1H), 7.45-7.37 (m, 2H), 7.01-6.91 (m, 2H), 6.86 (s, 1H), 4.06 (s, 2H), 2.86 (s, 2H), 2.72 (s, 3H), 1.40 (s, 6H).

### Example 233: 4-[2-(5-Fluoro-2-pyridyl)-5,5-dimethyl-4,6-dihydropyrrolo[1,2-b]pyrazol-3-yl]-1H-pyrrolo[2,3-b]pyridine

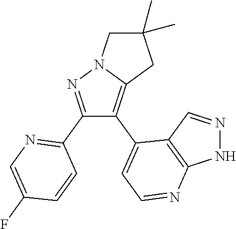 [SMILES: CC1(C)Cc2c(-c3ccnc4[nH]ncc34)c(-c3ccc(F)cn3)nn2C1]

To a solution of 3-bromo-2-(5-fluoropyridin-2-yl)-5,5-dimethyl-5,6-dihydro-4H-pyrrolo[1,2-b]pyrazole (Intermediate 152, 200 mg, 0.645 mmol), 4-(4,4,5,5-tetramethyl-1,3,2-dioxaborolan-2-yl)-1H-pyrrolo[2,3-b]pyridine (344 mg, 0.775 mmol), and Cs₂CO₃ (632 mg, 1.94 mmol) in 2-methyl-2-butanol (4 mL), and H₂O (1 mL), sparged with N₂ for 5 minutes, was added CataCXium® A Pd G3 (48 mg, 0.066 mmol). The reaction mixture was sparged with N₂ for an additional 5 minutes and heated to 90° C. employing microwave irradiation for 1 hour. The reaction mixture was cooled to room-temperature. The reaction mixture was poured into water (10 mL) and extracted with ethyl acetate (10 mL×3). The combined organic extracts were washed with brine (10 mL), dried over anhydrous Na₂SO₄, filtered, and concentrated to dryness under reduced pressure. Purification of the resulting residue (preparative HPLC using a Welch Xtimate C18 150×25 mm×5 μm column (eluent: 22% to 52% (v/v) CH₃CN and H₂O with 0.225% HCOOH) afforded the title compound (98.1 mg, 44%) as a white solid. MS (ESI): mass calcd. for C₂₀H₁₈FN₅ 347.2; m/z found 348.1 [M+H]⁺. ¹H NMR (400 MHz, DMSO-d₆): δ 11.52 (br s, 1H), 8.33-8.26 (m, 1H), 8.12-8.05 (m, 1H), 7.81-7.64 (m, 2H), 7.38-7.23 (m, 1H), 6.90-6.76 (m, 1H), 5.93-5.79 (m, 1H), 4.02 (s, 2H), 2.77 (s, 2H), 1.28 (s, 6H).

### Example 234: 4-[2-(5-Fluoro-2-pyridyl)-5,5-dimethyl-4,6-dihydropyrrolo[1,2-b]pyrazol-3-yl]-1H-pyrazolo[3,4-b]pyridine

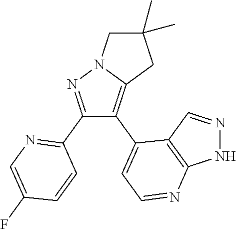 [SMILES: CC1(C)Cc2c(-c3ccnc4[nH]ncc34)c(-c3ccc(F)cn3)nn2C1]

The title compound was prepared in a manner analogous to Example 1, Steps A-B, except using 3-bromo-2-(5-fluoropyridin-2-yl)-5,5-dimethyl-5,6-dihydro-4H-pyrrolo[1,2-b]pyrazole (Intermediate 152) instead of 3-bromo-2-(5-fluoropyridin-2-yl)-6,7-dihydro-4H-pyrazolo[5,1-c][1,4]oxazine (Intermediate 37) and 4-(4,4,5,5-tetramethyl-1,3,2-dioxaborolan-2-yl)-1-((2-(trimethylsilyl)ethoxy)methyl)-1H-pyrazolo[3,4-b]pyridine (Intermediate 23) instead of 4-(4,4,5,5-tetramethyl-1,3,2-dioxaborolan-2-yl)-1-((2-(trimethylsilyl)ethoxy)methyl)-1H-pyrrolo[2,3-b]pyridine (Intermediate 21) in Step A. MS (ESI): mass calcd. for C₁₉H₁₇FN₆ 348.2; m/z found 349.2 [M+H]⁺. ¹H NMR (400 MHz, DMSO-d₆): δ 13.50 (br s, 1H), 8.40 (d, J=4.9 Hz, 1H), 8.27 (d, J=2.9 Hz, 1H), 7.87-7.74 (m, 2H), 7.40-7.32 (m, 1H), 7.00 (d, J=4.6 Hz, 1H), 4.04 (s, 2H), 2.85 (s, 2H), 1.29 (s, 6H).

### Example 235: 4-[2-(5-Fluoro-2-pyridyl)-5,5-dimethyl-4,6-dihydropyrrolo[1,2-b]pyrazol-3-yl]-6-methyl-1H-pyrrolo[2,3-b]pyridine

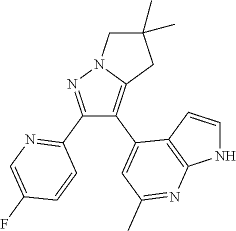 [SMILES: Cc1cc(-c2c(-c3ccc(F)cn3)nn3c2CC(C)(C)C3)c2cc[nH]c2n1]

Step A. 4-(2-(5-Fluoropyridin-2-yl)-5,5-dimethyl-5,6-dihydro-4H-pyrrolo[1,2-b]pyrazol-3-yl)-6-methyl-1-tosyl-1H-pyrrolo[2,3-b]pyridine. 3-Bromo-2-(5-fluoropyridin-2-yl)-5,5-dimethyl-5,6-dihydro-4H-pyrrolo[1,2-b]pyrazole (Intermediate 152, 135 mg, 0.437 mmol), 6-methyl-4-(4,4,5,5-tetramethyl-1,3,2-dioxaborolan-2-yl)-1-tosyl-1H-pyrrolo[2,3-b]pyridine (Intermediate 219, 150 mg, 0.364 mmol), Cs₂CO₃ (357 mg, 1.09 mmol), 2-methyl-2-butanol (4 mL), and H₂O (1 mL) were added to a 10 mL microwave tube. The resultant mixture was sparged with N₂ for 5 minutes and then treated with CataCXium® A Pd G3 (27 mg, 0.037 mmol). The resultant mixture was sparged with N₂ for another 5 minutes and heated at 90° C. via microwave irradiation for 1 hour. The reaction mixture was cooled to room-temperature. The reaction mixture was poured into water (10 mL) and extracted with ethyl acetate (10 mL×3). The combined organic extracts were washed with brine (10 mL), dried over anhydrous Na₂SO₄, filtered, and concentrated to dryness under reduced pressure. The resulting residue was purified by FCC (SiO₂, 0-33% EtOAc/petroleum ether) to afford the title compound (108 mg, 58%) as a yellow oil. MS (ESI): mass calcd. for C₂₈H₂₆FN₅₀₂S 515.2; m/z found 516.1 [M+H]⁺.

Step B. 4-(2-(5-Fluoropyridin-2-yl)-5,5-dimethyl-5,6-dihydro-4H-pyrrolo[1,2-b]pyrazol-3-yl)-6-methyl-1H-pyrrolo[2,3-b]pyridine. NaOH (1.0 mL, 2 M in water, 2.1 mmol) was added to a mixture consisting of 4-(2-(5-fluoropyridin-2-yl)-5,5-dimethyl-5,6-dihydro-4H-pyrrolo[1,2-b]pyrazol-3-yl)-6-methyl-1-tosyl-1H-pyrrolo[2,3-b]pyridine (108 mg, 0.209 mmol) and 1,4-dioxane (3 mL). The reaction mixture was stirred at 50° C. for 16 hours. The reaction mixture was cooled to room-temperature and concentrated to dryness under reduced pressure. Purification of the resulting residue (preparative HPLC using a Boston Prime C18 150×30 mm×5 μm column (eluent: 33% to 63% (v/v) CH₃CN and H₂O with 0.05% NH₃)) afforded the title compound (29.6 mg, 39%) as a white solid. MS (ESI): mass calcd. for C₂₁H₂₀FN₅ 361.2; m/z found 362.1 [M+H]⁺. ¹H NMR (400 MHz, CDCl₃): δ 9.85 (br s, 1H), 8.55-8.29 (m, 1H), 7.35-7.29 (m, 1H), 7.24-7.17 (m, 1H), 7.13-7.09 (m, 1H), 6.85 (s, 1H), 5.97 (d, J=3.2 Hz, 1H), 4.06 (s, 2H), 2.81 (s, 2H), 2.62 (s, 3H), 1.37 (s, 6H).

### Example 236: 5-Fluoro-4-[2-(5-fluoro-2-pyridyl)-5,5-dimethyl-4,6-dihydropyrrolo[1,2-b]pyrazol-3-yl]-1H-pyrazolo[3,4-b]pyridine

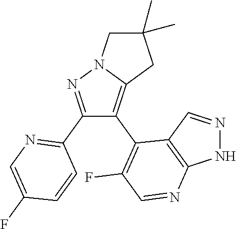 [SMILES: CC1(C)Cc2c(-c3c(F)cnc4[nH]ncc34)c(-c3ccc(F)cn3)nn2C1]

Step A. (2-(5-Fluoropyridin-2-yl)-5,5-dimethyl-5,6-dihydro-4H-pyrrolo[1,2-b]pyrazol-3-yl)boronic acid. n-BuLi (1.8 mL, 1.6 M in THF, 2.9 mmol) was added dropwise to a −5° C. (dry ice/water) solution consisting of 3-bromo-2-(5-fluoropyridin-2-yl)-5,5-dimethyl-5,6-dihydro-4H-pyrrolo[1,2-b]pyrazole (Intermediate 152, 300 mg, 0.967 mmol), 2-isopropoxy-4,4,5,5-tetramethyl-1,3,2-dioxaborolane (0.60 mL, 2.9 mmol) and dry THF (10 mL) under N₂. The resultant mixture was stirred for 1 hour. The reaction mixture was gradually warmed to room-temperature. The reaction mixture was quenched with H₂O (1 mL). The suspension isolated via filtration. The filter cake was washed with ethyl acetate (20 mL×3) before drying under reduced pressure to afford the title compound (400 mg, crude), which was used in the next step without further purification.

Step B. 5-Fluoro-4-(2-(5-fluoropyridin-2-yl)-5,5-dimethyl-5,6-dihydro-4H-pyrrolo[1,2-b]pyrazol-3-yl)-1-((2-(trimethylsilyl)ethoxy)methyl)-1H-pyrazolo[3,4-b]pyridine. (2-(5-Fluoropyridin-2-yl)-5,5-dimethyl-5,6-dihydro-4H-pyrrolo[1,2-b]pyrazol-3-yl)boronic acid (240 mg, crude), 5-fluoro-4-iodo-1-((2-(trimethylsilyl)ethoxy)methyl)-1H-pyrazolo[3,4-b]pyridine (Intermediate 204, 172 mg, 0.437 mmol), K₃PO₄ (278 mg, 1.31 mmol) and 1,4-dioxane (8 mL) and H₂O (2 mL) were added to a 20 mL microwave tube. The resultant mixture was sparged with N₂ for 5 minutes and then treated with Pd(dtbpf)Cl₂ (29 mg, 0.044 mmol). The resultant mixture was sparged with N₂ for another 5 minutes and heated at 90° C. via microwave irradiation for 1 hour. The reaction mixture was cooled to room-temperature. The reaction mixture was poured into water (20 mL) and extracted with ethyl acetate (20 mL×3). The combined organic extracts were washed with brine (20 mL), dried over anhydrous Na₂SO₄, filtered, and concentrated to dryness under reduced pressure. The resulting residue was purified by FCC (SiO₂, 0-25% EtOAc/petroleum ether) to afford the title compound (100 mg, 41%) as a yellow oil. MS (ESI): mass calcd. for C₂₅H₃₀F₂N₆OSi 496.2; m/z found 497.1 [M+H]⁺.

Step C. 5-Fluoro-4-[2-(5-fluoro-2-pyridyl)-5,5-dimethyl-4,6-dihydropyrrolo[1,2-b]pyrazol-3-yl]-1H-pyrazolo[3,4-b]pyridine. 5-Fluoro-4-(2-(5-fluoropyridin-2-yl)-5,5-dimethyl-5,6-dihydro-4H-pyrrolo[1,2-b]pyrazol-3-yl)-1-((2-(trimethylsilyl)ethoxy)methyl)-1H-pyrazolo[3,4-b]pyridine (150 mg, 0.302 mmol), TFA (1.5 mL), and dichloromethane (3 mL) were added to a 50 mL round-bottomed flask. The resultant solution was stirred at room-temperature for 2 hours. The reaction solution was concentrated to dryness under reduced pressure to afford the title compound, which was diluted with methanol (5 mL), and then ammonia 7 M in methanol (2 mL) was added. The resultant solution was stirred at room-temperature for 30 mins. The mixture was concentrated to dryness under reduced pressure. The resulting residue was purified by preparative HPLC using a Welch Xtimate C18 150*25 mm*5 μm column (eluent: 35% to 65% (v/v) CH₃CN and H₂O with 0.225% HCOOH) to afford still-impure product. The product was suspended in water (10 mL), the mixture frozen using dry ice/EtOH, and then lyophilized to dryness to afford the still-impure compound. The product was further purified by SFC over DAICEL CHIRALCEL OD-H 250 mm×30 mm×5 μm (isocratic elution: EtOH (containing 0.1% of 25% aq. NH₃): supercritical CO₂, 30%: 70% to 30%: 70% (v/v)). The pure fractions were collected and the volatiles were removed under reduced pressure. The product was suspended in water (10 mL), the mixture frozen using dry ice/EtOH, and then lyophilized to dryness to afford the title compound (17.6 mg, 16%) as a white solid. MS (ESI): mass calcd. for C₁₉H₁₆F₂N₆ 366.1; m/z found 367.1 [M+H]⁺. ¹H NMR (400 MHz, DMSO-d₆): δ 13.71 (br s, 1H), 8.47 (d, J=2.8 Hz, 1H), 8.23 (d, J=3.0 Hz, 1H), 8.03-7.86 (m, 1H), 7.84-7.71 (m, 1H), 7.66 (s, 1H), 4.06 (s, 2H), 2.76 (s, 2H), 1.29 (s, 6H).

### Example 237: 3-Chloro-4-(2-(5-fluoropyridin-2-yl)-5,5-dimethyl-5,6-dihydro-4H-pyrrolo[1,2-b]pyrazol-3-yl)-1H-pyrazolo[3,4-b]pyridine

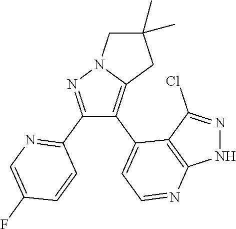 [SMILES: CC1(C)Cc2c(-c3ccnc4[nH]nc(Cl)c34)c(-c3ccc(F)cn3)nn2C1]

The title compound was prepared in a manner analogous to Example 224, except using 4-(2-(5-fluoropyridin-2-yl)-5,5-dimethyl-5,6-dihydro-4H-pyrrolo[1,2-b]pyrazol-3-yl)-1H-pyrazolo[3,4-b]pyridine (Example 234) instead of 5-fluoro-4-(2-(5-fluoropyridin-2-yl)-5,6-dihydro-4H-pyrrolo[1,2-b]pyrazol-3-yl)-6-methyl-1H-pyrazolo[3,4-b]pyridine (Example 9). MS (ESI): mass calcd. for C₁₉H₁₆ClFN₆ 382.1; m/z found 383.1 [M+H]⁺. ¹H NMR (400 MHz, CD₃OD): δ 8.50 (d, J=4.8 Hz, 1H), 8.10 (dt, J=3.0, 0.7 Hz, 1H), 7.79-7.65 (m, 1H), 7.55 (td, J=8.6, 2.9 Hz, 1H), 7.08 (d, J=4.8 Hz, 1H), 4.07 (s, 2H), 2.83 (d, J=15.9 Hz, 1H), 2.72 (d, J=15.9 Hz, 1H), 1.36 (d, J=10.9 Hz, 6H).

### Example 238: 4-[2-(5-Fluoro-2-pyridyl)-5,5-dimethyl-4,6-dihydropyrrolo[1,2-b]pyrazol-3-yl]-6-methyl-1H-pyrazolo[3,4-b]pyridine

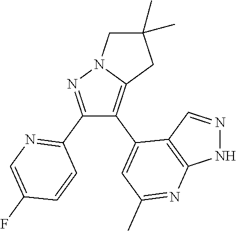 [SMILES: Cc1cc(-c2c(-c3ccc(F)cn3)nn3c2CC(C)(C)C3)c2cn[nH]c2n1]

The title compound was prepared in a manner analogous to Example 1, Steps A-B, except using 3-bromo-2-(5-fluoropyridin-2-yl)-5,5-dimethyl-5,6-dihydro-4H-pyrrolo[1,2-b]pyrazole (Intermediate 152) instead of Intermediate 37 and 6-methyl-4-(4,4,5,5-tetramethyl-1,3,2-dioxaborolan-2-yl)-1-((2-(trimethylsilyl)ethoxy)methyl)-1H-pyrazolo[3,4-b]pyridine (Intermediate 26) instead of 4-(4,4,5,5-tetramethyl-1,3,2-dioxaborolan-2-yl)-1-((2-(trimethylsilyl)ethoxy)methyl)-1H-pyrrolo[2,3-b]pyridine (Intermediate 21) in Step A. MS (ESI): mass calcd. for C₂₀H₁₉FN₆ 362.2; m/z found 363.2 [M+H]⁺. ¹H NMR (400 MHz, DMSO-d₆): δ 13.26 (br s, 1H), 8.27 (d, J=2.7 Hz, 1H), 7.91-7.69 (m, 2H), 7.19 (s, 1H), 6.92 (s, 1H), 4.15-3.92 (m, 2H), 2.86 (s, 2H), 2.53 (s, 3H), 1.29 (s, 6H).

### Example 239: 6-(Difluoromethyl)-4-[2-(5-fluoro-2-pyridyl)-5,5-dimethyl-4,6-dihydropyrrolo[1,2-b]pyrazol-3-yl]-1H-pyrazolo[3,4-b]pyridine

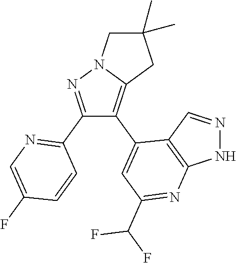 [SMILES: CC1(C)Cc2c(-c3cc(C(F)F)nc4[nH]ncc34)c(-c3ccc(F)cn3)nn2C1]

The title compound was prepared in a manner analogous to Example 1, Steps A-B, except using 3-bromo-2-(5-fluoropyridin-2-yl)-5,5-dimethyl-5,6-dihydro-4H-pyrrolo[1,2-b]pyrazole (Intermediate 152) instead of 3-bromo-2-(5-fluoropyridin-2-yl)-6,7-dihydro-4H-pyrazolo[5,1-c][1,4]oxazine (Intermediate 37); and using 6-(difluoromethyl)-4-(4,4,5,5-tetramethyl-1,3,2-dioxaborolan-2-yl)-1-((2-(trimethylsilyl)ethoxy)methyl)-1H-pyrazolo[3,4-b]pyridine (Intermediate 221) instead of 4-(4,4,5,5-tetramethyl-1,3,2-dioxaborolan-2-yl)-1-((2-(trimethylsilyl)ethoxy)methyl)-1H-pyrrolo[2,3-b]pyridine (Intermediate 21) in Step A. MS (ESI): mass calcd. for C₂₀H₁₇F₃N₆ 398.1; m/z found 399.1 [M+H]⁺. ¹H NMR (400 MHz, CDCl₃): δ 11.65 (br s, 1H), 8.39-8.14 (m, 1H), 7.79-7.65 (m, 1H), 7.54-7.49 (m, 1H), 7.44-7.34 (m, 2H), 6.93-6.59 (m, 1H), 4.09 (s, 2H), 2.89 (s, 2H), 1.40 (s, 6H).

### Example 240: 5-Fluoro-4-[2-(5-fluoro-2-pyridyl)-5,5-dimethyl-4,6-dihydropyrrolo[1,2-b]pyrazol-3-yl]-6-methyl-1H-pyrazolo[3,4-b]pyridine

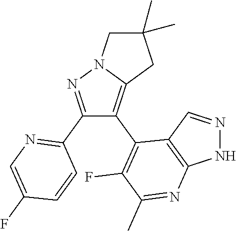 [SMILES: Cc1nc2[nH]ncc2c(-c2c(-c3ccc(F)cn3)nn3c2CC(C)(C)C3)c1F]

The title compound was prepared in a manner analogous to Example 1, Steps A-B, except using 3-bromo-2-(5-fluoropyridin-2-yl)-5,5-dimethyl-5,6-dihydro-4H-pyrrolo[1,2-b]pyrazole (Intermediate 152) instead of 3-bromo-2-(5-fluoropyridin-2-yl)-6,7-dihydro-4H-pyrazolo[5,1-c][1,4]oxazine (Intermediate 37); and using 5-fluoro-6-methyl-4-(4,4,5,5-tetramethyl-1,3,2-dioxaborolan-2-yl)-1-((2-(trimethylsilyl)ethoxy)methyl)-1H-pyrazolo[3,4-b]pyridine (Intermediate 223) instead of 4-(4,4,5,5-tetramethyl-1,3,2-dioxaborolan-2-yl)-1-((2-(trimethylsilyl)ethoxy)methyl)-1H-pyrrolo[2,3-b]pyridine (Intermediate 21) in Step A. MS (ESI): mass calcd. for C₂₀H₁₈F₂N₆, 380.2; m/z found 381.1 [M+H]⁺. ¹H NMR (400 MHz, CDCl₃): δ 11.24 (br s, 1H), 8.45-8.12 (m, 1H), 7.88-7.60 (m, 1H), 7.46-7.31 (m, 2H), 4.09 (s, 2H), 2.82 (s, 2H), 2.68 (d, J=3.5 Hz, 3H), 1.39 (s, 6H).

### Example 241: 4-[2-(4-Fluorophenyl)-5,5-bis(methyl-d₃)-4,6-dihydropyrrolo[1,2-b]pyrazol-3-yl]-1H-pyrazolo[3,4-b]pyridine

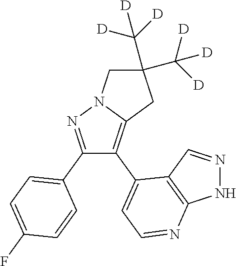 [SMILES: [2H]C([2H])([2H])C1(C([2H])([2H])[2H])Cc2c(-c3ccnc4[nH]ncc34)c(-c3ccc(F)cc3)nn2C1]

The title compound was prepared in a manner analogous to Example 1, Steps A-B, except using 3-bromo-2-(4-fluorophenyl)-5,5-bis(methyl-d₃)-5, 6-dihydro-4H-pyrrolo[1,2-b]pyrazole (Intermediate 153) instead of 3-bromo-2-(5-fluoropyridin-2-yl)-6,7-dihydro-4H-pyrazolo[5,1-c][1,4]oxazine (Intermediate 37); and using 4-(4,4,5,5-tetramethyl-1,3,2-dioxaborolan-2-yl)-1-((2-(trimethylsilyl)ethoxy)methyl)-1H-pyrazolo[3,4-b]pyridine (Intermediate 23) instead of 4-(4,4,5,5-tetramethyl-1,3,2-dioxaborolan-2-yl)-1-((2-(trimethylsilyl)ethoxy)methyl)-1H-pyrrolo[2,3-b]pyridine (Intermediate 21) in Step A. MS (ESI): mass calcd. for C₂₀H₁₂D₆FN₅, 353.2; m/z found 354.2 [M+H]⁺. ¹H NMR (400 MHz, CDCl₃): δ 12.07 (br s, 1H), 8.51 (d, J=4.6 Hz, 1H), 7.57 (s, 1H), 7.48-7.34 (m, 2H), 7.09-6.84 (m, 3H), 4.06 (s, 2H), 2.86 (s, 2H).

### Example 242: 4-[2-(4-Fluorophenyl)-5,5-bis(methyl-d₃)-4,6-dihydropyrrolo[1,2-b]pyrazol-3-yl]-6-methyl-1H-pyrazolo[3,4-b]pyridine

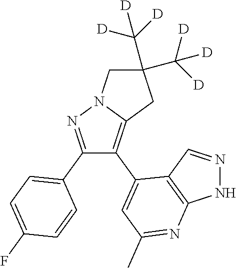 [SMILES: [2H]C([2H])([2H])C1(C([2H])([2H])[2H])Cc2c(-c3cc(C)nc4[nH]ncc34)c(-c3ccc(F)cc3)nn2C1]

The title compound was prepared in a manner analogous to Example 1, Steps A-B, except using 3-bromo-2-(4-fluorophenyl)-5,5-bis(methyl-d₃)-5,6-dihydro-4H-pyrrolo[1,2-b]pyrazole (Intermediate 153) instead of 3-bromo-2-(5-fluoropyridin-2-yl)-6,7-dihydro-4H-pyrazolo[5,1-c][1,4]oxazine (Intermediate 37); and using 6-methyl-4-(4,4,5,5-tetramethyl-1,3,2-dioxaborolan-2-yl)-1-((2-(trimethylsilyl)ethoxy)methyl)-1H-pyrazolo[3,4-b]pyridine (Intermediate 26) instead of 4-(4,4,5,5-tetramethyl-1,3,2-dioxaborolan-2-yl)-1-((2-(trimethylsilyl)ethoxy)methyl)-1H-pyrrolo[2,3-b]pyridine (Intermediate 21) in Step A. MS (ESI): mass calcd. for C₂₁H₁₄D₆FN₅, 367.2; m/z found 368.5 [M+H]⁺. ¹H NMR (400 MHz, CDCl₃): δ 7.46 (s, 1H), 7.44-7.37 (m, 2H), 7.01-6.91 (m, 2H), 6.85 (s, 1H), 4.05 (s, 2H), 2.85 (s, 2H), 2.67 (s, 3H). N—H proton not observed.

### Example 243: 5-Fluoro-4-[2-(5-fluoro-2-pyridyl)-5,5-bis(methyl-d₃)-4,6-dihydropyrrolo[1,2-b]pyrazol-3-yl]-1H-pyrazolo[3,4-b]pyridine

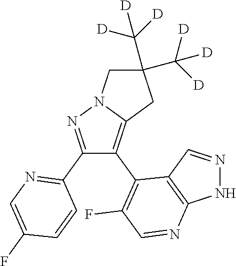 [SMILES: [2H]C([2H])([2H])C1(C([2H])([2H])[2H])Cc2c(-c3c(F)cnc4[nH]ncc34)c(-c3ccc(F)cn3)nn2C1]

The title compound was prepared in a manner analogous to Example 1, Steps A-B, except using 3-bromo-2-(5-fluoropyridin-2-yl)-5,5-bis(methyl-d₃)-5, 6-dihydro-4H-pyrrolo[1,2-b]pyrazole (Intermediate 154) instead of 3-bromo-2-(5-fluoropyridin-2-yl)-6,7-dihydro-4H-pyrazolo[5,1-c][1,4]oxazine (Intermediate 37); and using 5-fluoro-4-(4,4,5,5-tetramethyl-1,3,2-dioxaborolan-2-yl)-1-((2-(trimethylsilyl)ethoxy)methyl)-1H-pyrazolo[3,4-b]pyridine (Intermediate 222) instead of 4-(4,4,5,5-tetramethyl-1,3,2-dioxaborolan-2-yl)-1-((2-(trimethylsilyl)ethoxy)methyl)-1H-pyrrolo[2,3-b]pyridine (Intermediate 21) in Step A. MS (ESI): mass calcd. for C₁₉H₁₀D₆F₂N₆, 372.2; m/z found 373.1 [M+H]⁺. ¹H NMR (400 MHz, DMSO-d₆): δ 13.70 (br s, 1H), 8.47 (d, J=2.7 Hz, 1H), 8.22 (d, J=2.9 Hz, 1H), 8.00-7.87 (m, 1H), 7.83-7.70 (m, 1H), 7.66 (s, 1H), 4.06 (s, 2H), 2.75 (s, 2H).

### Example 244: 5-Fluoro-4-[2-(5-fluoro-2-pyridyl)-5,5-bis(methyl-d₃)-4,6-dihydropyrrolo[1,2-b]pyrazol-3-yl]-6-methyl-1H-pyrazolo[3,4-b]pyridine

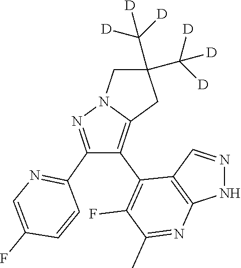 [SMILES: [2H]C([2H])([2H])C1(C([2H])([2H])[2H])Cc2c(-c3c(F)c(C)nc4[nH]ncc34)c(-c3ccc(F)cn3)nn2C1]

The title compound was prepared in a manner analogous to Example 1, Steps A-B, except using 3-bromo-2-(5-fluoropyridin-2-yl)-5,5-bis(methyl-d₃)-5, 6-dihydro-4H-pyrrolo[1,2-b]pyrazole (Intermediate 154) instead of 3-bromo-2-(5-fluoropyridin-2-yl)-6,7-dihydro-4H-pyrazolo[5,1-c][1,4]oxazine (Intermediate 37); and 5-fluoro-6-methyl-4-(4,4,5,5-tetramethyl-1,3,2-dioxaborolan-2-yl)-1-((2-(trimethylsilyl)ethoxy)methyl)-1H-pyrazolo[3,4-b]pyridine (Intermediate 223) instead of 4-(4,4,5,5-tetramethyl-1,3,2-dioxaborolan-2-yl)-1-((2-(trimethylsilyl)ethoxy)methyl)-1H-pyrrolo[2,3-b]pyridine (Intermediate 21) in Step A. MS (ESI): mass calcd. for C₂₀H₁₂D₆F₂N₆ 386.2; m/z found 387.3 [M+H]⁺. ¹H NMR (400 MHz, CDCl₃): δ 10.82 (br s, 1H), 8.23 (d, J=2.7 Hz, 1H), 7.82-7.58 (m, 1H), 7.41-7.30 (m, 2H), 4.08 (s, 2H), 2.81 (s, 2H), 2.67 (d, J=3.5 Hz, 3H).

### Example 245: 4-[2-(4-Fluorophenyl)-4,4-dimethyl-5,6-dihydropyrrolo[1,2-b]pyrazol-3-yl]-1H-pyrazolo[3,4-b]pyridine

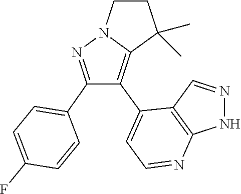 [SMILES: CC1(C)CCn2nc(-c3ccc(F)cc3)c(-c3ccnc4[nH]ncc34)c21]

The title compound was prepared in a manner analogous to Example 1, Steps A-B, except using 3-bromo-2-(5-fluoropyridin-2-yl)-4,4-dimethyl-5,6-dihydro-4H-pyrrolo[1,2-b]pyrazole (Intermediate 156) instead of 3-bromo-2-(5-fluoropyridin-2-yl)-6,7-dihydro-4H-pyrazolo[5,1-c][1,4]oxazine (Intermediate 37); using 4-(4,4,5,5-tetramethyl-1,3,2-dioxaborolan-2-yl)-1-((2-(trimethylsilyl)ethoxy)methyl)-1H-pyrazolo[3,4-b]pyridine (Intermediate 23) instead of 4-(4,4,5,5-tetramethyl-1,3,2-dioxaborolan-2-yl)-1-((2-(trimethylsilyl)ethoxy)methyl)-1H-pyrrolo[2,3-b]pyridine (Intermediate 21); and Pd(dppf)Cl₂ instead of CataCXium® A Pd G3 in Step A. MS (ESI): mass calcd. for C₂₀H₁₈FN₅ 347.2; m/z found 348.1 [M+H]⁺. ¹H NMR (400 MHz, DMSO-d₆): δ 13.61 (s, 1H), 8.55 (d, J=4.6 Hz, 1H), 7.41 (s, 1H), 7.28-7.17 (m, 2H), 7.13 (d, J=4.8 Hz, 1H), 7.07-6.94 (m, 2H), 4.27 (t, J=7.0 Hz, 2H), 2.42 (t, J=6.9 Hz, 2H), 1.20 (s, 6H).

### Example 246: 4-[2-(4-Fluorophenyl)-4,4-dimethyl-5,6-dihydropyrrolo[1,2-b]pyrazol-3-yl]-6-methyl-1H-pyrazolo[3,4-b]pyridine

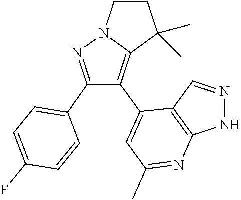 [SMILES: Cc1cc(-c2c(-c3ccc(F)cc3)nn3c2C(C)(C)CC3)c2cn[nH]c2n1]

The title compound was prepared in a manner analogous to Example 1, Steps A-B, except using 3-bromo-2-(5-fluoropyridin-2-yl)-4,4-dimethyl-5,6-dihydro-4H-pyrrolo[1,2-b]pyrazole (Intermediate 156) instead of 3-bromo-2-(5-fluoropyridin-2-yl)-6,7-dihydro-4H-pyrazolo[5,1-c][1,4]oxazine (Intermediate 37); using 6-methyl-4-(4,4,5,5-tetramethyl-1,3,2-dioxaborolan-2-yl)-1-((2-(trimethylsilyl)ethoxy)methyl)-1H-pyrazolo[3,4-b]pyridine (Intermediate 26) instead of 4-(4,4,5,5-tetramethyl-1,3,2-dioxaborolan-2-yl)-1-((2-(trimethylsilyl)ethoxy)methyl)-1H-pyrrolo[2,3-b]pyridine (Intermediate 21); and Pd(dppf)Cl₂ instead of CcataCXium® A Pd G3 in Step A. MS (ESI): mass calcd. for C₂₁H₂₀FN₅ 361.2; m/z found 362.2 [M+H]⁺. ¹H NMR (400 MHz, DMSO-d₆): δ 13.38 (br s, 1H), 7.30 (s, 1H), 7.28-7.18 (m, 2H), 7.06-6.93 (m, 3H), 4.26 (t, J=6.9 Hz, 2H), 2.61 (s, 3H), 2.45-2.35 (m, 2H), 1.21 (s, 6H).

### Example 247: 2-(4-Fluorophenyl)-3-(1H-pyrazolo[3,4-b]pyridin-4-yl)spiro[4,6-dihydropyrrolo[1,2-b]pyrazole-5,1′-cyclopropane]

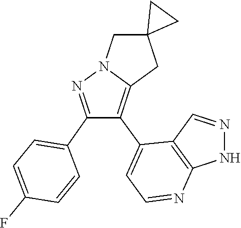 [SMILES: Fc1ccc(-c2nn3c(c2-c2ccnc4[nH]ncc24)CC2(CC2)C3)cc1]

The title compound was prepared in a manner analogous to Example 1, Steps A-B, except using 3′-bromo-2′-(4-fluorophenyl)-4′H,6′H-spiro[cyclopropane-1,5′-pyrrolo[1,2-b]pyrazole](Intermediate 157) instead of 3-bromo-2-(5-fluoropyridin-2-yl)-6,7-dihydro-4H-pyrazolo[5,1-c][1,4]oxazine (Intermediate 37); using 4-(4,4,5,5-tetramethyl-1,3,2-dioxaborolan-2-yl)-1-((2-(trimethylsilyl)ethoxy)methyl)-1H-pyrazolo[3,4-b]pyridine (Intermediate 23) instead of 4-(4,4,5,5-tetramethyl-1,3,2-dioxaborolan-2-yl)-1-((2-(trimethylsilyl)ethoxy)methyl)-1H-pyrrolo[2,3-b]pyridine (Intermediate 21); and Pd(dppf)Cl₂ instead of CataCXium® A Pd G3 in Step A. MS (ESI): mass calcd. for C₂₀H₁₆FN₅ 345.1; m/z found 346.0 [M+H]⁺. ¹H NMR (400 MHz, DMSO-d₆): δ 13.57 (s, 1H), 8.42 (d, J=4.9 Hz, 1H), 7.46 (s, 1H), 7.39-7.32 (m, 2H), 7.16-7.08 (m, 2H), 7.00 (d, J=4.8 Hz, 1H), 4.20 (s, 2H), 3.04 (s, 2H), 0.89-0.84 (m, 2H), 0.83-0.78 (m, 2H).

### Example 248: 2-(4-Fluorophenyl)-3-(6-methyl-1H-pyrazolo[3,4-b]pyridin-4-yl)spiro[4,6-dihydropyrrolo[1,2-b]pyrazole-5,1′-cyclopropane]

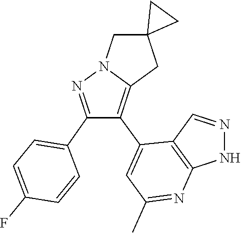 [SMILES: Cc1cc(-c2c(-c3ccc(F)cc3)nn3c2CC2(CC2)C3)c2cn[nH]c2n1]

The title compound was prepared in a manner analogous to Example 1, Steps A-B, except using 3′-bromo-2′-(4-fluorophenyl)-4′H,6′H-spiro[cyclopropane-1,5′-pyrrolo[1,2-b]pyrazole](Intermediate 157) instead of 3-bromo-2-(5-fluoropyridin-2-yl)-6,7-dihydro-4H-pyrazolo[5,1-c][1,4]oxazine (Intermediate 37); using 6-methyl-4-(4,4,5,5-tetramethyl-1,3,2-dioxaborolan-2-yl)-1-((2-(trimethylsilyl)ethoxy)methyl)-1H-pyrazolo[3,4-b]pyridine (Intermediate 26) instead of 4-(4,4,5,5-tetramethyl-1,3,2-dioxaborolan-2-yl)-1-((2-(trimethylsilyl)ethoxy)methyl)-1H-pyrrolo[2,3-b]pyridine (Intermediate 21); and Pd(dppf)Cl₂ instead of CataCXium® A Pd G3 in Step A. MS (ESI): mass calcd. for C₂₁H₁₈FN₅ 359.2; m/z found 360.1 [M+H]⁺. ¹H NMR (400 MHz, DMSO-d₆): δ 13.32 (s, 1H), 7.39-7.33 (m, 2H), 7.25 (s, 1H), 7.15-7.07 (m, 2H), 6.94 (s, 1H), 4.18 (s, 2H), 3.04 (s, 2H), 2.52 (s, 3H), 0.89-0.83 (m, 2H), 0.82-0.77 (m, 2H).

### Example 249: 2-(5-Fluoro-2-pyridyl)-3-(1H-pyrazolo[3,4-b]pyridin-4-yl)spiro[4,5-dihydropyrrolo[1,2-b]pyrazole-6,1′-cyclopropane]

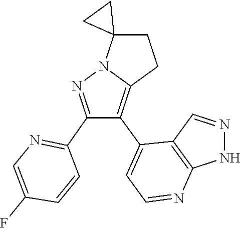 [SMILES: Fc1ccc(-c2nn3c(c2-c2ccnc4[nH]ncc24)CCC32CC2)nc1]

The title compound was prepared in a manner analogous to 4-(6,6-difluoro-2-(4-fluorophenyl)-4,5,6,7-tetrahydropyrazolo[1,5-a]pyridin-3-yl)-5-fluoro-1H-pyrazolo[3,4-b]pyridine (Example 280) Steps A-B, except using 4-(4,4,5,5-tetramethyl-1,3,2-dioxaborolan-2-yl)-1-((2-(trimethylsilyl)ethoxy)methyl)-1H-pyrazolo[3,4-b]pyridine (Intermediate 23) instead of 5-fluoro-4-(4,4,5,5-tetramethyl-1,3,2-dioxaborolan-2-yl)-1-((2-(trimethylsilyl)ethoxy)methyl)-1H-pyrazolo[3,4-b]pyridine (Intermediate 222); and using 3′-bromo-2′-(5-fluoropyridin-2-yl)-4′,5′-dihydrospiro[cyclopropane-1,6′-pyrrolo[1,2-b]pyrazole] (Intermediate 158) instead of 3-bromo-6,6-difluoro-2-(4-fluorophenyl)-4,5,6,7-tetrahydropyrazolo[1,5-a]pyridine (Intermediate 52) in Step A. MS (ESI): mass calcd. for C₁₉H₁₅FN₆ 346.1; m/z found 347.1 [M+H]⁺. ¹H NMR (500 MHz, Chloroform-d): δ 8.46 (d, J=6.0 Hz, 1H), 8.17 (d, J=2.9 Hz, 1H), 7.87 (dd, J=8.8, 4.4 Hz, 1H), 7.71 (s, 1H), 7.52-7.43 (m, 1H), 7.34 (d, J=5.8 Hz, 1H), 3.22 (dd, J=8.2, 6.7 Hz, 2H), 2.92-2.83 (m, 2H), 1.72-1.56 (m, 2H), 1.17-1.03 (m, 2H). N—H proton not observed.

### Example 250: (*S)-1′,1′-Difluoro-2-(4-fluorophenyl)-3-(1H-pyrazolo[3,4-b]pyridin-4-yl)spiro[4,6-dihydropyrrolo[1,2-b]pyrazole-5,2′-cyclopropane]

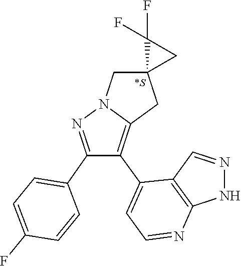 [SMILES: Fc1ccc(-c2nn3c(c2-c2ccnc4[nH]ncc24)C[C@@]2(C3)CC2(F)F)cc1]

The title compound was prepared in a manner analogous to Example 1 Steps A-B, except using 3′-bromo-2,2-difluoro-2′-(4-fluorophenyl)-4′,6′-dihydrospiro[cyclopropane-1,5′-pyrrolo[1,2-b]pyrazole] (Intermediate 159) instead of 3-bromo-2-(5-fluoropyridin-2-yl)-6,7-dihydro-4H-pyrazolo[5,1-c][1,4]oxazine (Intermediate 37); and using 4-(4,4,5,5-tetramethyl-1,3,2-dioxaborolan-2-yl)-1-((2-(trimethylsilyl)ethoxy)methyl)-1H-pyrazolo[3,4-b]pyridine (Intermediate 23) instead of 4-(4,4,5,5-tetramethyl-1,3,2-dioxaborolan-2-yl)-1-((2-(trimethylsilyl)ethoxy)methyl)-1H-pyrrolo[2,3-b]pyridine (Intermediate 21). Chiral SFC purification (DAICEL CHIRALPAK AD 250 mm×30 mm, 10 μm (isocratic elution: I (containing 0.1% of 25% aq. NH₃): supercritical CO₂, 45%: 55% to 45%: 55% (v/v)) afforded the title compound: MS (ESI): mass calcd. for C₂₀H₁₄F₃N₅ 381.1; m/z found 382.2 [M+H]⁺; ¹H NMR (400 MHz, DMSO-d₆): δ 13.62 (s, 1H), 8.45 (d, J=4.8 Hz, 1H), 7.48 (s, 1H), 7.41-7.33 (m, 2H), 7.19-7.10 (m, 2H), 7.04 (d, J=4.8 Hz, 1H), 4.49-4.40 (m, 2H), 3.33-3.17 (m, 2H), 2.02-1.82 (m, 2H) and (*R)-1′,1′-difluoro-2-(4-fluorophenyl)-3-(1H-pyrazolo[3,4-b]pyridin-4-yl)spiro[4,6-dihydropyrrolo[1,2-b]pyrazole-5,2′-cyclopropane] (Example 251).

### Example 251: (*R)-1′,1′-Difluoro-2-(4-fluorophenyl)-3-(1H-pyrazolo[3,4-b]pyridin-4-yl)spiro[4,6-dihydropyrrolo[1,2-b]pyrazole-5,2′-cyclopropane]

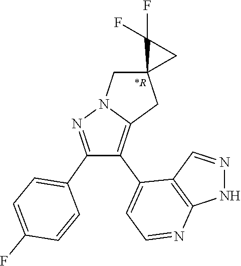 [SMILES: Fc1ccc(-c2nn3c(c2-c2ccnc4[nH]ncc24)C[C@]2(C3)CC2(F)F)cc1]

The title compound was isolated from chiral SFC purification of Example 250. MS (ESI): mass calcd. for C₂₀H₁₄F₃N₅, 381.1; m/z found, 382.1 [M+H]⁺. ¹H NMR (400 MHz, DMSO-d₆): δ 13.61 (s, 1H), 8.45 (d, J=4.6 Hz, 1H), 7.47 (s, 1H), 7.40-7.32 (m, 2H), 7.21-7.09 (m, 2H), 7.03 (d, J=4.8 Hz, 1H), 4.52-4.38 (m, 2H), 3.32-3.15 (m, 2H), 2.04-1.81 (m, 2H).

### Example 252: (*S)-1′,1′-Difluoro-2-(4-fluorophenyl)-3-(6-methyl-1H-pyrazolo[3,4-b]pyridin-4-yl)spiro[4,6-dihydropyrrolo[1,2-b]pyrazole-5,2′-cyclopropane]

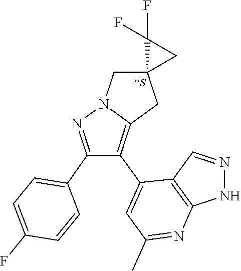 [SMILES: Cc1cc(-c2c(-c3ccc(F)cc3)nn3c2C[C@@]2(C3)CC2(F)F)c2cn[nH]c2n1]

The title compound was prepared in a manner analogous to Example 1, Steps A-B, except using bromo-2,2-difluoro-2′-(4-fluorophenyl)-4′,6′-dihydrospiro[cyclopropane-1,5′-pyrrolo[1,2-b]pyrazole] (Intermediate 159) instead of 3-bromo-2-(5-fluoropyridin-2-yl)-6,7-dihydro-4H-pyrazolo[5,1-c][1,4]oxazine (Intermediate 37); and 1-(4-methoxybenzyl)-6-methyl-4-(4,4,5,5-tetramethyl-1,3,2-dioxaborolan-2-yl)-1H-pyrazolo[3,4-b]pyridine (Intermediate 217) instead of 4-(4,4,5,5-tetramethyl-1,3,2-dioxaborolan-2-yl)-1-((2-(trimethylsilyl)ethoxy)methyl)-1H-pyrrolo[2,3-b]pyridine (Intermediate 21) in Step A. Purification (via SFC (DAICEL CHIRALPAK AD 250 mm×30 mm, 10 μm (isocratic elution: I (containing 0.1% of 25% aq. NH₃): supercritical CO₂, 45%: 55% to 45%: 55% (v/v))) afforded the title compound: MS (ESI): mass calcd. For C₂₁H₁₆F₃N₅ 395.1; m/z found 396.1 [M+H]⁺; ¹H NMR (400 MHz, CDCl₃): δ 7.54-7.37 (m, 3H), 7.03-6.86 (m, 3H), 4.56 (d, J=11.3 Hz, 1H), 4.39-4.28 (m, 1H), 3.39 (d, J=16.7 Hz, 1H), 3.14-3.05 (m, 1H), 2.76 (s, 3H), 1.79-1.59 (m, 2H). N—H proton not observed; and (*R)-1′,1′-difluoro-2-(4-fluorophenyl)-3-(6-methyl-1H-pyrazolo[3,4-b]pyridin-4-yl)spiro[4,6-dihydropyrrolo[1,2-b]pyrazole-5,2′-cyclopropane] (Example 253).

### Example 253: (*R)-1′.1′-Difluoro-2-(4-fluorophenyl)-3-(6-methyl-1H-pyrazolo[3,4-b]pyridin-4-yl)spiro[4,6-dihydropyrrolo[1,2-b]pyrazole-5,2′-cyclopropane]

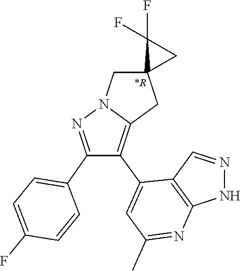

The title compound was isolated from chiral SFC purification of Example 252. MS (ESI): mass calcd. for C₂₁H₁₆F₃N₅ 395.1; m/z found 396.1 [M+H]⁺. ¹H NMR (400 MHz, CDCl₃): δ 7.50-7.36 (m, 3H), 7.05-6.84 (m, 3H), 4.56 (d, J=11.3 Hz, 1H), 4.39-4.28 (m, 1H), 3.39 (d, J=16.7 Hz, 1H), 3.16-3.05 (m, 1H), 2.75 (s, 3H), 1.79-1.61 (m, 2H). N—H proton not observed.

### Example 254: (*S)-1′1l′-Difluoro-2-(5-fluoro-2-pyridyl)-3-(1H-pyrazolo[3,4-b]pyridin-4-yl)spiro[4,6-dihydropyrrolo[1,2-b]pyrazole-5,2′-cyclopropane]

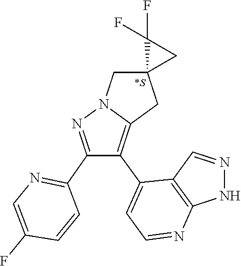 [SMILES: Fc1ccc(-c2nn3c(c2-c2ccnc4[nH]ncc24)C[C@@]2(C3)CC2(F)F)nc1]

The title compound was prepared in a manner analogous to Example 1, Steps A-B, except using 3′-bromo-2,2-difluoro-2′-(5-fluoropyridin-2-yl)-4′,6′-dihydrospiro[cyclopropane-1,5′-pyrrolo[1,2-b]pyrazole] (Intermediate 160) instead of 3-bromo-2-(5-fluoropyridin-2-yl)-6,7-dihydro-4H-pyrazolo[5,1-c][1,4]oxazine (Intermediate 37); and 4-(4,4,5,5-tetramethyl-1,3,2-dioxaborolan-2-yl)-1-((2-(trimethylsilyl)ethoxy)methyl)-1H-pyrazolo[3,4-b]pyridine (Intermediate 23) instead of 4-(4,4,5,5-tetramethyl-1,3,2-dioxaborolan-2-yl)-1-((2-(trimethylsilyl)ethoxy)methyl)-1H-pyrrolo[2,3-b]pyridine (Intermediate 21) in Step A. The title compound was purified by chiral SFC (DAICEL CHIRALPAK AD 250 mm×30 mm, 10 μm (isocratic elution: IPA (containing 0.1% of 25% aq. NH₃): supercritical CO₂, 45%: 55% to 55%: 45% (v/v)): MS (ESI): mass calcd. for C₁₉H₁₃F₃N₆ 382.1; m/z found 383.0 [M+H]⁺; ¹H NMR (400 MHz, DMSO-d₆): δ 13.52 (br s, 1H), 8.42 (d, J=4.9 Hz, 1H), 8.29 (d, J=2.7 Hz, 1H), 7.89-7.84 (m, 1H), 7.83-7.76 (m, 1H), 7.39 (s, 1H), 7.05 (d, J=4.9 Hz, 1H), 4.51-4.41 (m, 2H), 3.31-3.16 (m, 2H), 2.02-1.83 (m, 2H); and *R-1′,1′-difluoro-2-(5-fluoro-2-pyridyl)-3-(1H-pyrazolo[3,4-b]pyridin-4-yl)spiro[4,6-dihydropyrrolo[1,2-b]pyrazole-5,2′-cyclopropane] (Example 255).

### Example 255: (*R)-1′.1′-Difluoro-2-(5-fluoro-2-pyridyl)-3-(1H-pyrazolo[3,4-b]pyridin-4-yl)spiro[4,6-dihydropyrrolo[1,2-b]pyrazole-5,2′-cyclopropane]

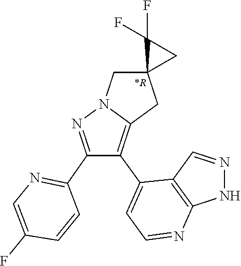 [SMILES: Fc1ccc(-c2nn3c(c2-c2ccnc4[nH]ncc24)C[C@]2(C3)CC2(F)F)nc1]

The title compound was isolated from chiral SFC purification of Example 254. MS (ESI): mass calcd. for C₁₉H₁₃F₃N₆ 382.1; m/z found 383.0 [M+H]⁺. ¹H NMR (400 MHz, DMSO-d₆): δ 13.53 (br s, 1H), 8.42 (d, J=4.6 Hz, 1H), 8.29 (d, J=2.9 Hz, 1H), 7.89-7.84 (m, 1H), 7.83-7.76 (m, 1H), 7.39 (s, 1H), 7.05 (d, J=4.9 Hz, 1H), 4.51-4.41 (m, 2H), 3.32-3.16 (m, 2H), 2.02-1.83 (m, 2H).

### Example 256: (*S)-3-(3-Chloro-1H-pyrazolo[3,4-b]pyridin-4-yl)-1′,1′-difluoro-2-(5-fluoro-2-pyridyl)spiro[4,6-dihydropyrrolo[1,2-b]pyrazole-5,2′-cyclopropane]

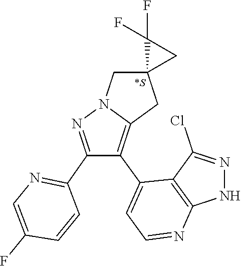 [SMILES: Fc1ccc(-c2nn3c(c2-c2ccnc4[nH]nc(Cl)c24)C[C@@]2(C3)CC2(F)F)nc1]

Step A. 2,2-Difluoro-2′-(5-fluoropyridin-2-yl)-3′-(1H-pyrazolo[3,4-b]pyridin-4-yl)-4′H,6′H-spiro[cyclopropane-1,5′-pyrrolo[1,2-b]pyrazole]. The title compound was prepared in a manner analogous to Example 1, Steps A-B, except using 3′-bromo-2,2-difluoro-2′-(5-fluoropyridin-2-yl)-4′,6′-dihydrospiro[cyclopropane-1,5′-pyrrolo[1,2-b]pyrazole] (Intermediate 160) instead of 3-bromo-2-(5-fluoropyridin-2-yl)-6,7-dihydro-4H-pyrazolo[5,1-c][1,4]oxazine (Intermediate 37); and 4-(4,4,5,5-tetramethyl-1,3,2-dioxaborolan-2-yl)-1-((2-(trimethylsilyl)ethoxy)methyl)-1H-pyrazolo[3,4-b]pyridine (Intermediate 23) instead of 4-(4,4,5,5-tetramethyl-1,3,2-dioxaborolan-2-yl)-1-((2-(trimethylsilyl)ethoxy)methyl)-1H-pyrrolo[2,3-b]pyridine (Intermediate 21) in Step A. MS (ESI): mass calcd. for C₁₉H₁₃F₃N₆, 382.1; m/z found, 383.0 [M+H]⁺.

Step B. (*R)-3-(3-Chloro-1H-pyrazolo[3,4-b]pyridin-4-yl)-1′,1′-difluoro-2-(5-fluoro-2-pyridyl)spiro[4,6-dihydropyrrolo[1,2-b]pyrazole-5,2′-cyclopropane]. 2,2-Difluoro-2′-(5-fluoropyridin-2-yl)-3′-(1H-pyrazolo[3,4-b]pyridin-4-yl)-4′H,6′H-spiro[cyclopropane-1,5′-pyrrolo[1,2-b]pyrazole] (500 mg) and N-chlorosuccinimide (262 mg, 1.96 mmol) were dissolved in DMF (4 mL). The reaction mixture was stirred at r.t. for 16 hours. Additional N-chlorosuccinimide (200 mg) was added and the reaction mixture, and the reaction mixture was stirred at r.t. for 16 hours. The reaction mixture was partitioned between water and EtOAc, the aqueous layer was extracted with EtOAc (2×), and the combined organics were washed with brine, dried (Na₂SO₄), and concentrated under reduced pressure. Purification (preparative HPLC using a Boston Green ODS C18 150×30 mm×5 μm column (eluent: 38% to 68% (v/v) CH₃CN and H₂O with 0.225% HCOOH)), then resolution with chiral SFC (DAICEL CHIRALCEL OJ-H 250 mm×30 mm, 5 μm (isocratic elution: I (containing 0.1% of 25% aq. NH₃): supercritical CO₂, 20%: 80% to 20%: 80% (v/v))) afforded the title compound and (*R)-3-(3-chloro-1H-pyrazolo[3,4-b]pyridin-4-yl)-1′,1′-difluoro-2-(5-fluoro-2-pyridyl)spiro[4,6-dihydropyrrolo[1,2-b]pyrazole-5,2′-cyclopropane] (Example 257). MS (ESI): mass calcd. for C₁₉H₁₂ClF₃N₆, 416.1; m/z found 417.0 [M+H]⁺. ¹H NMR (400 MHz, DMSO-d₆): δ 13.80 (d, J=7.4 Hz, 1H), 8.51 (d, J=4.5 Hz, 1H), 8.18 (dd, J=2.9, 5.6 Hz, 1H), 7.93 (dd, J=4.6, 8.8 Hz, 1H), 7.82-7.69 (m, 2H), 4.59-4.40 (m, 2H), 3.27-2.96 (m, 2H), 2.03-1.80 (m, 2H).

### Example 257: (*R)-3-(3-Chloro-1H-pyrazolo[3,4-b]pyridin-4-yl)-1′,1′-difluoro-2-(5-fluoro-2-pyridyl)spiro[4,6-dihydropyrrolo[1,2-b]pyrazole-5,2′-cyclopropane]

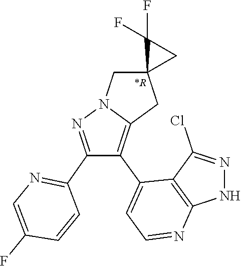 [SMILES: Fc1ccc(-c2nn3c(c2-c2ccnc4[nH]nc(Cl)c24)C[C@]2(C3)CC2(F)F)nc1]

The title compound was isolated from chiral SFC purification of Example 256. MS (ESI): mass calcd. for C₁₉H₁₂ClF₃N₆ 416.1; m/z found 417.0 [M+H]⁺. ¹H NMR (400 MHz, DMSO-d₆): δ 13.80 (br s, 1H), 8.51 (d, J=4.5 Hz, 1H), 8.18 (dd, J=3.0, 5.7 Hz, 1H), 7.93 (dd, J=4.6, 8.8 Hz, 1H), 7.83-7.68 (m, 2H), 4.54-4.45 (m, 2H), 3.27-2.93 (m, 2H), 2.04-1.79 (m, 2H).

### Example 258: (*S)-2,2-Difluoro-3′-(5-fluoro-1H-pyrazolo[3,4-b]pyridin-4-yl)-2′-(5-fluoropyridin-2-yl)-4′H,6′H-spiro[cyclopropane-1,5′-pyrrolo[1,2-b]pyrazole]

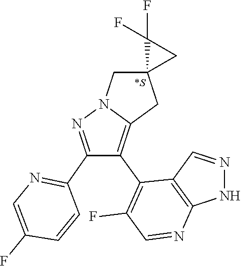 [SMILES: Fc1ccc(-c2nn3c(c2-c2c(F)cnc4[nH]ncc24)C[C@@]2(C3)CC2(F)F)nc1]

A mixture of the title compound was prepared in a manner analogous to 5-fluoro-4-[2-(5-fluoro-2-pyridyl)-5,6-dihydro-4H-pyrrolo[1,2-b]pyrazol-3-yl]-1H-pyrazolo[3,4-b]pyridine (Example 221), except using 3′-bromo-2,2-difluoro-2′-(5-fluoropyridin-2-yl)-4′,6′-dihydrospiro[cyclopropane-1,5′-pyrrolo[1,2-b]pyrazole (Intermediate 160) instead of 3-bromo-2-(5-fluoropyridin-2-yl)-5,6-dihydro-4H-pyrrolo[1,2-b]pyrazole (Intermediate 39); 5-fluoro-4-iodo-1-(tetrahydro-2H-pyran-2-yl)-1H-pyrazolo[3,4-b]pyridine (Intermediate 230) instead of lithium 2-(2′-(4-fluorophenyl)-5′H,7′H-spiro[cyclopropane-1,6′-pyrazolo[5,1-b][1,3]oxazin]-3′-yl)-2-hydroxy-4,4,5,5-tetramethyl-1,3,2-dioxaborolan-2-uide (Intermediate 231), and 4N HCl in dioxane as a deprotecting reagent instead of TFA, ammonia, and methanol. Purification (chiral SFC (DAICEL CHIRALPAK AD 250 mm×30 mm, 10 μm (isocratic elution: EtOH (containing 0.1% of 25% aq. NH₃): supercritical CO₂, 35%: 65% to 35%: 65% (v/v)) afforded the title compound: MS (ESI): mass calcd. for C₁₉H₁₂F₄N₆ 400.1; m/z found 401.5 [M+H]⁺; ¹H NMR (400 MHz, DMSO-d₆): δ 13.72 (s, 1H), 8.48 (d, J=2.9 Hz, 1H), 8.24 (d, J=2.9 Hz, 1H), 7.94 (dd, J=4.5, 8.8 Hz, 1H), 7.84-7.75 (m, 1H), 7.72 (d, J=1.3 Hz, 1H), 4.55-4.42 (m, 2H), 3.27-3.07 (m, 2H), 2.02-1.83 (m, 2H); and (*R)-1′,1′-difluoro-3-(5-fluoro-1H-pyrazolo[3,4-b]pyridin-4-yl)-2-(5-fluoro-2-pyridyl)spiro[4,6-dihydropyrrolo[1,2-b]pyrazole-5,2′-cyclopropane] (Example 259).

### Example 259: (*R)-1′.1′-Difluoro-3-(5-fluoro-1H-pyrazolo[3,4-b]pyridin-4-yl)-2-(5-fluoro-2-pyridyl)spiro[4,6-dihydropyrrolo[1,2-b]pyrazole-5,2′-cyclopropane]

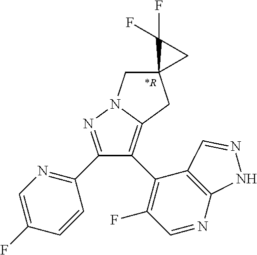 [SMILES: Fc1ccc(-c2nn3c(c2-c2c(F)cnc4[nH]ncc24)C[C@]2(C3)CC2(F)F)nc1]

The title compound was isolated from chiral SFC purification of Example 258. MS (ESI): mass calcd. for C₁₉H₁₂F₄N₆ 400.1; m/z found 401.4 [M+H]⁺. ¹H NMR (400 MHz, DMSO-d₆): δ 13.72 (br s, 1H), 8.49 (d, J=2.9 Hz, 1H), 8.25 (d, J=3.0 Hz, 1H), 7.94 (dd, J=4.5, 8.8 Hz, 1H), 7.83-7.76 (m, 1H), 7.72 (s, 1H), 4.53-4.45 (m, 2H), 3.28-3.07 (m, 2H), 2.02-1.83 (m, 2H).

### Example 260: (*S)-1′,1′-Difluoro-2-(5-fluoro-2-pyridyl)-3-(6-methyl-1H-pyrazolo[3,4-b]pyridin-4-yl)spiro[4,6-dihydropyrrolo[1,2-b]pyrazole-5,2′-cyclopropane]

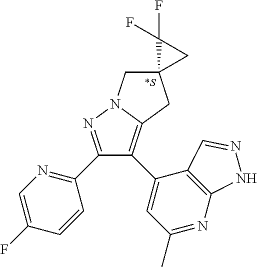 [SMILES: Cc1cc(-c2c(-c3ccc(F)cn3)nn3c2C[C@@]2(C3)CC2(F)F)c2cn[nH]c2n1]

The title compound was prepared in a manner analogous to Example 1, Steps A-B, except using 3′-bromo-2,2-difluoro-2′-(5-fluoropyridin-2-yl)-4′,6′-dihydrospiro[cyclopropane-1,5′-pyrrolo[1,2-b]pyrazole] (Intermediate 160) instead of 3-bromo-2-(5-fluoropyridin-2-yl)-6,7-dihydro-4H-pyrazolo[5,1-c][1,4]oxazine (Intermediate 37) and 6-methyl-4-(4,4,5,5-tetramethyl-1,3,2-dioxaborolan-2-yl)-1-((2-(trimethylsilyl)ethoxy)methyl)-1H-pyrazolo[3,4-b]pyridine (Intermediate 26) instead of 4-(4,4,5,5-tetramethyl-1,3,2-dioxaborolan-2-yl)-1-((2-(trimethylsilyl)ethoxy)methyl)-1H-pyrrolo[2,3-b]pyridine (Intermediate 21) in Step A. The title compound and (*R)-1′,1′-difluoro-2-(5-fluoro-2-pyridyl)-3-(6-methyl-1H-pyrazolo[3,4-b]pyridin-4-yl)spiro[4,6-dihydropyrrolo[1,2-b]pyrazole-5,2′-cyclopropane] (Example 261) were isolated via SFC purification (DAICEL CHIRALPAK AD 250 mm×30 mm, 10 um (eluent: 55% to 55% (v/v) supercritical CO₂ in EtOH and H₂O with 0.1% NH₃)). MS (ESI): mass calcd. for C₂₀H₁₅F₃N₆ 396.1; m/z found 397.0 [M+H]⁺. ¹H NMR (400 MHz, DMSO-d₆): δ 13.27 (s, 1H), 8.28 (d, J=2.74 Hz, 1H), 7.75-7.89 (m, 2H), 7.20 (s, 1H), 6.97 (s, 1H), 4.40-4.50 (m, 2H), 3.29-3.32 (m, 1H), 3.17-3.24 (m, 1H), 2.54 (s, 3H), 1.83-2.00 (m, 2H).

### Example 261: (*R)-1′,1′-Difluoro-2-(5-fluoro-2-pyridyl)-3-(6-methyl-1H-pyrazolo[3,4-b]pyridin-4-yl)spiro[4,6-dihydropyrrolo[1,2-b]pyrazole-5,2′-cyclopropane]

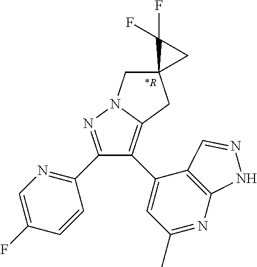 [SMILES: Cc1cc(-c2c(-c3ccc(F)cn3)nn3c2C[C@]2(C3)CC2(F)F)c2cn[nH]c2n1]

The title compound was isolated from chiral SFC purification of Example 260. MS (ESI): mass calcd. for C₂₀H₁₅F₃N₆ 396.1; m/z found 397.0 [M+H]⁺. ¹H NMR (400 MHz, DMSO-d₆): δ 13.28 (s, 1H), 8.28 (d, J=2.86 Hz, 1H), 7.74-7.89 (m, 2H), 7.16-7.24 (m, 1H), 6.98 (s, 1H), 4.39-4.53 (m, 2H), 3.29-3.32 (m, 1H), 3.16-3.26 (m, 1H), 2.54 (s, 3H), 1.83-2.01 (m, 2H).

### Example 262: (*S)-3′-(3-Chloro-6-methyl-1H-pyrazolo[3,4-b]pyridin-4-yl)-2,2-difluoro-2′-(5-fluoropyridin-2-yl)-4′H,6′H-spiro[cyclopropane-1,5′-pyrrolo[1,2-b]pyrazole]

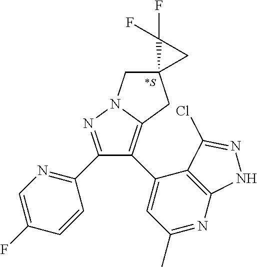 [SMILES: Cc1cc(-c2c(-c3ccc(F)cn3)nn3c2C[C@@]2(C3)CC2(F)F)c2c(Cl)n[nH]c2n1]

The title compound was prepared in a manner analogous to Example 224, except using *S-1′,1′-difluoro-2-(5-fluoro-2-pyridyl)-3-(6-methyl-1H-pyrazolo[3,4-b]pyridin-4-yl)spiro[4,6-dihydropyrrolo[1,2-b]pyrazole-5,2′-cyclopropane] (Example 260) instead of 5-fluoro-4-(2-(5-fluoropyridin-2-yl)-5, 6-dihydro-4H-pyrrolo[1,2-b]pyrazol-3-yl)-6-methyl-1H-pyrazolo[3,4-b]pyridine (Example 9). After two hours, additional NaH (60% in mineral oil, 0.78 equiv) and N-chlorosuccinimide (0.83 equiv) was added. MS (ESI): mass calcd. for C₂₀H₁₄ClF₃N₆ 430.1; m/z found, 431.1 [M+H]⁺. ¹H NMR (400 MHz, CD₃OD): δ 8.18-8.05 (m, 1H), 7.84-7.72 (m, 1H), 7.62-7.48 (m, 1H), 7.06 (d, J=7.5 Hz, 1H), 4.52 (dd, J=11.4, 7.0 Hz, 1H), 4.46-4.34 (m, 1H), 3.36 (d, J=16.7 Hz, 1H), 2.98 (dd, J=16.7, 4.0 Hz, 1H), 2.64 (d, J=4.5 Hz, 3H), 1.92-1.63 (m, 2H). N—H proton not observed.

### Example 263: (*R)-1′.1′-Difluoro-3-(5-fluoro-6-methyl-1H-pyrazolo[3,4-b]pyridin-4-yl)-2-(5-fluoro-2-pyridyl)spiro[4,6-dihydropyrrolo[1,2-b]pyrazole-5,2′-cyclopropane]

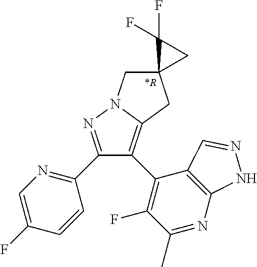 [SMILES: Cc1nc2[nH]ncc2c(-c2c(-c3ccc(F)cn3)nn3c2C[C@]2(C3)CC2(F)F)c1F]

The title compound was prepared in a manner analogous to 3-bromo-2-(5-fluoropyridin-2-yl)-5,6-dihydro-4H-pyrrolo[1,2-b]pyrazole (Example 221 Z56), except using 3′-bromo-2,2-difluoro-2′-(5-fluoropyridin-2-yl)-4′,6′-dihydrospiro[cyclopropane-1,5′-pyrrolo[1,2-b]pyrazole (Intermediate 160) instead of 3-bromo-2-(5-fluoropyridin-2-yl)-5,6-dihydro-4H-pyrrolo[1,2-b]pyrazole (Intermediate 39) and 5-fluoro-4-iodo-6-methyl-1-((2-(trimethylsilyl)ethoxy)methyl)-1H-pyrazolo[3,4-b]pyridine (Intermediate 205) instead of lithium 2-(2′-(4-fluorophenyl)-5′H,7′H-spiro[cyclopropane-1,6′-pyrazolo[5,1-b][1,3]oxazin]-3′-yl)-2-hydroxy-4,4,5,5-tetramethyl-1,3,2-dioxaborolan-2-uide (Intermediate 231). The title compound and (*S)-1′,1′-difluoro-3-(5-fluoro-6-methyl-1H-pyrazolo[3,4-b]pyridin-4-yl)-2-(5-fluoro-2-pyridyl)spiro[4,6-dihydropyrrolo[1,2-b]pyrazole-5,2′-cyclopropane] (Example 264) were resolved by chiral SFC (DAICEL CHIRALPAK AD 250 mm×30 mm, 10 μm (isocratic elution: EtOH (containing 0.1% of 25% aq. NH₃): supercritical CO₂, 55%: 45% to 55%: 45% (v/v))). MS (ESI): mass calcd. for C₂₀H₁₄F₄N₆ 414.1; m/z found 415.1 [M+H]⁺. ¹H NMR (400 MHz, DMSO-d₆): δ 13.49 (s, 1H), 8.24 (d, J=2.9 Hz, 1H), 7.97-7.88 (m, 1H), 7.84-7.73 (m, 1H), 7.53 (m, 1H), 4.48 (s, 2H), 3.22-3.06 (m, 2H), 2.55-2.51 (m, 3H), 2.02-1.82 (m, 2H).

### Example 264: (*S)-1′.1′-Difluoro-3-(5-fluoro-6-methyl-1H-pyrazolo[3,4-b]pyridin-4-yl)-2-(5-fluoro-2-pyridyl)spiro[4,6-dihydropyrrolo[1,2-b]pyrazole-5,2′-cyclopropane]

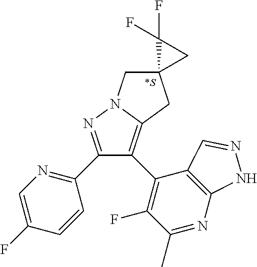 [SMILES: Cc1nc2[nH]ncc2c(-c2c(-c3ccc(F)cn3)nn3c2C[C@@]2(C3)CC2(F)F)c1F]

The title compound was isolated from chiral SFC purification of Example 263. MS (ESI): mass calcd. for C₂₀H₁₄F₄N₆ 414.1; m/z found 415.1 [M+H]⁺. ¹H NMR (400 MHz, DMSO-d₆): δ 13.49 (s, 1H), 8.24 (d, J=2.9 Hz, 1H), 7.96-7.89 (m, 1H), 7.83-7.74 (m, 1H), 7.55-7.51 (m, 1H), 4.53-4.42 (m, 2H), 3.25-3.07 (m, 2H), 2.52-2.51 (m, 3H), 2.01-1.83 (m, 2H).

### Example 265: (*R)-1′.1′-Difluoro-3-(5-fluoro-3,6-dimethyl-1H-pyrazolo[3,4-b]pyridin-4-yl)-2-(5-fluoro-2-pyridyl)spiro[4,6-dihydropyrrolo[1,2-b]pyrazole-5,2′-cyclopropane]

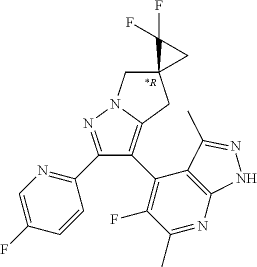 [SMILES: Cc1nc2[nH]nc(C)c2c(-c2c(-c3ccc(F)cn3)nn3c2C[C@]2(C3)CC2(F)F)c1F]

A mixture of the title compound was prepared in a manner analogous to 3-bromo-2-(5-fluoropyridin-2-yl)-5,6-dihydro-4H-pyrrolo[1,2-b]pyrazole (Example 221), except using 3′-bromo-2,2-difluoro-2′-(5-fluoropyridin-2-yl)-4′,6′-dihydrospiro[cyclopropane-1,5′-pyrrolo[1,2-b]pyrazole (Intermediate 160) instead of 3-bromo-2-(5-fluoropyridin-2-yl)-5,6-dihydro-4H-pyrrolo[1,2-b]pyrazole (Intermediate 39); and 4-bromo-5-fluoro-6-methyl-1-((2-(trimethylsilyl)ethoxy)methyl)-1H-pyrazolo[3,4-b]pyridine (Intermediate 208) instead of lithium 2-(2′-(4-fluorophenyl)-5′H,7′H-spiro[cyclopropane-1,6′-pyrazolo[5,1-b][1,3]oxazin]-3′-yl)-2-hydroxy-4,4,5,5-tetramethyl-1,3,2-dioxaborolan-2-uide (Intermediate 231). The title compound and (*S)-1′,1′-Difluoro-3-(5-fluoro-3,6-dimethyl-1H-pyrazolo[3,4-b]pyridin-4-yl)-2-(5-fluoro-2-pyridyl)spiro[4,6-dihydropyrrolo[1,2-b]pyrazole-5,2′-cyclopropane] (Example 266) were resolved by chiral SFC (DAICEL CHIRALPAK AD 250 mm×30 mm, 10 μm (isocratic elution: i-PrOH (containing 0.1% of 25% aq. NH₃): supercritical CO₂, 30%: 70% to 30%: 70% (v/v))). MS (ESI): mass calcd. for C₂₁H₁₆F₄N₆ 428.1; m/z found 429.1 [M+H]⁺. ¹H NMR (400 MHz, DMSO-d₆) δ 13.08 (s, 1H), 8.14 (d, J=2.9 Hz, 1H), 7.96 (dd, J=4.5, 8.8 Hz, 1H), 7.76-7.69 (m, 1H), 4.57-4.43 (m, 2H), 3.18-2.89 (m, 2H), 2.53 (d, J=3.5 Hz, 3H), 2.00-1.83 (m, 5H).

### Example 266: (*S)-1′1l′-Difluoro-3-(5-fluoro-3,6-dimethyl-1H-pyrazolo[3,4-b]pyridin-4-yl)-2-(5-fluoro-2-pyridyl)spiro[4,6-dihydropyrrolo[1,2-b]pyrazole-5,2′-cyclopropane]

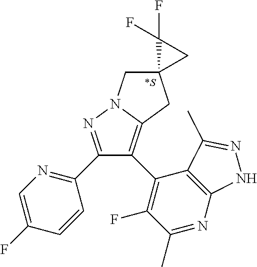 [SMILES: Cc1nc2[nH]nc(C)c2c(-c2c(-c3ccc(F)cn3)nn3c2C[C@@]2(C3)CC2(F)F)c1F]

The title compound was isolated from chiral SFC purification of Example 265. MS (ESI): mass calcd. for C₂₁H₁₆F₄N₆ 428.1; m/z found 429.1 [M+H]⁺. ¹H NMR (400 MHz, DMSO-d₆): δ 13.09 (s, 1H), 8.14 (d, J=3.0 Hz, 1H), 7.96 (dd, J=4.5, 8.9 Hz, 1H), 7.76-7.69 (m, 1H), 4.49 (s, 2H), 3.08-2.94 (m, 2H), 2.52 (d, J=3.5 Hz, 3H), 2.02-1.84 (m, 5H).

### Example 267: (4aR,5aR)-2-(4-Fluorophenyl)-3-(1H-pyrazolo[3,4-b]pyridin-4-yl)-4,4a,5,5a-tetrahydrocyclopropa[4,5]pyrrolo[1,2-b]pyrazole

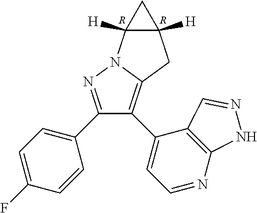

The title compound was prepared in a manner analogous to Example 1, Steps A-B, except using (4aR,5aR)-3-bromo-2-(4-fluorophenyl)-4,4a,5,5a-tetrahydrocyclopropa[4,5]pyrrolo[1,2-b]pyrazole (Intermediate 162) instead of 3-bromo-2-(5-fluoropyridin-2-yl)-6,7-dihydro-4H-pyrazolo[5,1-c][1,4]oxazine (Intermediate 37), 4-(4,4,5,5-tetramethyl-1,3,2-dioxaborolan-2-yl)-1-((2-(trimethylsilyl)ethoxy)methyl)-1H-pyrazolo[3,4-b]pyridine (Intermediate 23) instead of 4-(4,4,5,5-tetramethyl-1,3,2-dioxaborolan-2-yl)-1-((2-(trimethylsilyl)ethoxy)methyl)-1H-pyrrolo[2,3-b]pyridine (Intermediate 21), and Pd(dppf)Cl₂ instead of CataCXium A Pd G3 in Step A. MS (ESI): mass calcd. for C₁₉H₁₄FN₅, 331.1; m/z found, 332.0 [M+H]⁺. ¹H NMR (400 MHz, DMSO-d₆): δ 13.57 (br s, 1H), 8.42 (d, J=4.8 Hz, 1H), 7.46 (s, 1H), 7.37-7.28 (m, 2H), 7.16-7.07 (m, 2H), 6.97 (d, J=4.8 Hz, 1H), 4.28-4.17 (m, 1H), 3.33-3.30 (m, 1H), 2.98 (d, J=16.8 Hz, 1H), 2.36-2.24 (m, 1H), 1.24-1.13 (m, 1H), 0.63-0.54 (m, 1H).

### Example 268: (4aS,5aS)-2-(4-Fluorophenyl)-3-(1H-pyrazolo[3,4-b]pyridin-4-yl)-4,4a,5,5a-tetrahydrocyclopropa[4,5]pyrrolo[1,2-b]pyrazole

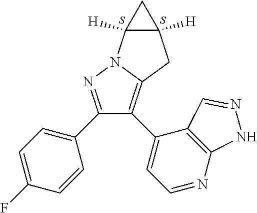

The title compound was prepared in a manner analogous to Example 1, Steps A-B, except using (4aS,5aS)-3-bromo-2-(4-fluorophenyl)-4,4a,5,5a-tetrahydrocyclopropa[4,5]pyrrolo[1,2-b]pyrazole (Intermediate 161) instead of 3-bromo-2-(5-fluoropyridin-2-yl)-6,7-dihydro-4H-pyrazolo[5,1-c][1,4]oxazine (Intermediate 37), 4-(4,4,5,5-tetramethyl-1,3,2-dioxaborolan-2-yl)-1-((2-(trimethylsilyl)ethoxy)methyl)-1H-pyrazolo[3,4-b]pyridine (Intermediate 23) instead of 4-(4,4,5,5-tetramethyl-1,3,2-dioxaborolan-2-yl)-1-((2-(trimethylsilyl)ethoxy)methyl)-1H-pyrrolo[2,3-b]pyridine (Intermediate 21), and Pd(dppf)Cl₂ instead of CataCXium® A Pd G3 in Step A. MS (ESI): mass calcd. for C₁₉H₁₄FN₅ 331.1; m/z found 332.0 [M+H]⁺. ¹H NMR (400 MHz, DMSO-d₆): δ 13.58 (br s, 1H), 8.43 (d, J=4.4 Hz, 1H), 7.46 (s, 1H), 7.37-7.31 (m, 2H), 7.16-7.08 (m, 2H), 6.98 (d, J=4.8 Hz, 1H), 4.27-4.19 (m, 1H), 3.35-3.29 (m, 1H), 3.01-2.95 (m, 1H), 2.35-2.25 (m, 1H), 1.25-1.15 (m, 1H), 0.62-0.56 (m, 1H).

### Example 269: (4aR,5aR)-2-(4-Fluorophenyl)-3-(6-methyl-1-((2-(trimethylsilyl)ethoxy)methyl)-1H-pyrazolo[3,4-b]pyridin-4-yl)-4,4a,5,5a-tetrahydrocyclopropa[4,5]pyrrolo[1,2-b]pyrazole

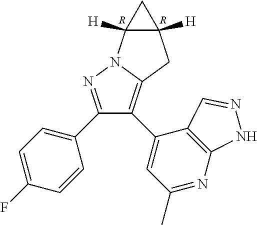

The title compound was prepared in a manner analogous to Example 1, Steps A-B, except using 6-methyl-4-(4,4,5,5-tetramethyl-1,3,2-dioxaborolan-2-yl)-1-((2-(trimethylsilyl)ethoxy)methyl)-1H-pyrazolo[3,4-b]pyridine (Intermediate 26) instead of 4-(4,4,5,5-tetramethyl-1,3,2-dioxaborolan-2-yl)-1-((2-(trimethylsilyl)ethoxy)methyl)-1H-pyrrolo[2,3-b]pyridine (Intermediate 21)(4aR,5aR)-3-Bromo-2-(4-fluorophenyl)-4,4a,5,5a-tetrahydrocyclopropa[4,5]pyrrolo[1,2-b]pyrazole (Intermediate 162) instead of 3-bromo-2-(5-fluoropyridin-2-yl)-6,7-dihydro-4H-pyrazolo[5,1-c][1,4]oxazine (Intermediate 37), and Pd(dppf)Cl₂ instead of CataCXium® A Pd G3 in Step A. MS (ESI): mass calcd. for C₂₀H₁₆FN₅ 345.1; m/z found 346.1 [M+H]⁺. ¹H NMR (400 MHz, DMSO-d₆): δ 13.33 (s, 1H), 7.36-7.27 (m, 3H), 7.14-7.06 (m, 2H), 6.91 (s, 1H), 4.27-4.19 (m, 1H), 3.32-3.29 (m, 1H), 2.99 (d, J=16.8 Hz, 1H), 2.52 (s, 3H), 2.34-2.21 (m, 1H), 1.26-1.11 (m, 1H), 0.61-0.50 (m, 1H).

### Example 270: (4aS,5aS)-2-(4-Fluorophenyl)-3-(6-methyl-1-((2-(trimethylsilyl)ethoxy)methyl)-1H-pyrazolo[3,4-b]pyridin-4-yl)-4,4a,5,5a-tetrahydrocyclopropa[4,5]pyrrolo[1,2-b]pyrazole

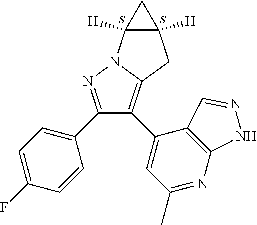

The title compound was prepared in a manner analogous to Example 1, Steps A-B, except using 6-methyl-4-(4,4,5,5-tetramethyl-1,3,2-dioxaborolan-2-yl)-1-((2-(trimethylsilyl)ethoxy)methyl)-1H-pyrazolo[3,4-b]pyridine (Intermediate 26) instead of 4-(4,4,5,5-tetramethyl-1,3,2-dioxaborolan-2-yl)-1-((2-(trimethylsilyl)ethoxy)methyl)-1H-pyrrolo[2,3-b]pyridine (Intermediate 21)(4aS,5aS)-3-bromo-2-(4-fluorophenyl)-4,4a,5,5a-tetrahydrocyclopropa[4,5]pyrrolo[1,2-b]pyrazole (Intermediate 161) instead of 3-bromo-2-(5-fluoropyridin-2-yl)-6,7-dihydro-4H-pyrazolo[5,1-c][1,4]oxazine (Intermediate 37), and Pd(dppf)Cl₂ instead of CataCXium® A Pd G3 in Step A. MS (ESI): mass calcd. for C₂₀H₁₆FN₅ 345.1; m/z found 346.1 [M+H]⁺. ¹H NMR (400 MHz, DMSO-d₆): δ 13.34 (s, 1H), 7.38-7.30 (m, 2H), 7.29 (s, 1H), 7.15-7.06 (m, 2H), 6.91 (s, 1H), 4.24 (t, J=5.6 Hz, 1H), 3.34-3.28 (m, 1H), 2.99 (d, J=16.8 Hz, 1H), 2.53 (s, 3H), 2.39-2.25 (m, 1H), 1.25-1.15 (m, 1H), 0.63-0.54 (m, 1H).

### Example 271: (4aS,5aS)-3-(3-Chloro-1H-pyrazolo[3,4-b]pyridin-4-yl)-2-(5-fluoropyridin-2-yl)-4,4a,5,5a-tetrahydrocyclopropa[4,5]pyrrolo[1,2-b]pyrazole

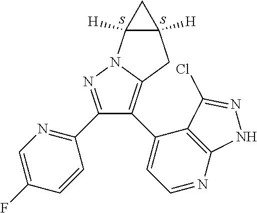

The title compound was prepared in a manner analogous to Example 224, except using (4aS,5aS)-2-(5-fluoropyridin-2-yl)-3-(1H-pyrazolo[3,4-b]pyridin-4-yl)-4,4a,5,5a-tetrahydrocyclopropa[4,5]pyrrolo[1,2-b]pyrazole (Example 26) instead of 5-fluoro-4-(2-(5-fluoropyridin-2-yl)-5, 6-dihydro-4H-pyrrolo[1,2-b]pyrazol-3-yl)-6-methyl-1H-pyrazolo[3,4-b]pyridine (Example 9). MS (ESI): mass calcd. for C₁₈H₁₂ClFN₆ 366.1; m/z found 367.1 [M+H]⁺. ¹H NMR (400 MHz, CD₃OD): δ 8.49 (dd, J=4.8, 4.1 Hz, 1H), 8.15-8.04 (m, 1H), 7.81-7.66 (m, 1H), 7.63-7.47 (m, 1H), 7.09 (dd, J=8.4, 4.8 Hz, 1H), 4.21 (t, J=6.1 Hz, 1H), 3.27-3.09 (m, 1H), 2.97 (dd, J=53.3, 17.1 Hz, 1H), 2.35 (p, J=6.0 Hz, 1H), 1.29 (dt, J=8.7, 5.9 Hz, 1H), 0.72-0.49 (m, 1H). N—H proton not observed.

### Example 272: (4aS,5aS)-3-(5-Fluoro-1H-pyrazolo[3,4-b]pyridin-4-yl)-2-(5-fluoropyridin-2-yl)-4,4a,5,5a-tetrahydrocyclopropa[4,5]pyrrolo[1,2-b]pyrazole

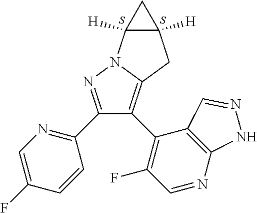

The title compound was prepared in a manner analogous to (5a*S,6a*R)-6,6-difluoro-3-(5-fluoro-1H-pyrazolo[3,4-b]pyridin-4-yl)-2-(5-fluoropyridin-2-yl)-5,5a,6,6a-tetrahydro-4H-cyclopropa[e]pyrazolo[1,5-a]pyridine (Example 358), except using and (4aS,5aS)-3-Bromo-2-(5-fluoropyridin-2-yl)-4,4a,5,5a-tetrahydrocyclopropa[4,5]pyrrolo[1,2-b]pyrazole (Intermediate 48) instead of (5a*S,6a*R)-6,6-difluoro-3-bromo-2-(5-fluoropyridin-2-yl)-5,5a,6,6a-tetrahydro-4H-cyclopropa[e]pyrazolo[1,5-a]pyridine (Intermediate 182). MS (ESI): mass calcd. for C₁₈H₁₂F₂N₆ 350.1; m/z found 351.2 [M+H]⁺. ¹H NMR (400 MHz, CDCl₃): δ 10.41 (s, 1H), 8.44 (d, J=2.8 Hz, 1H), 8.16 (d, J=2.9 Hz, 1H), 7.75 (dd, J=8.8, 4.4 Hz, 1H), 7.42 (s, 1H), 7.36 (td, J=8.4, 2.9 Hz, 1H), 4.18 (t, J=5.9 Hz, 1H), 3.25 (dd, J=17.3, 6.6 Hz, 1H), 2.99 (d, J=17.3 Hz, 1H), 2.38-2.20 (m, 1H), 1.34-1.14 (m, 1H), 0.82-0.64 (m, 1H).

### Example 273: (4aS,5aS)-3-(5-Fluoro-6-methyl-1H-pyrazolo[3,4-b]pyridin-4-yl)-2-(5-fluoropyridin-2-yl)-4,4a,5,5a-tetrahydrocyclopropa[4,5]pyrrolo[1,2-b]pyrazole

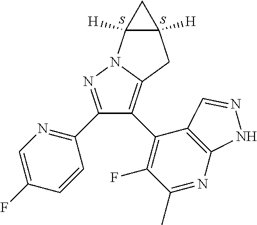

The title compound was prepared in a manner analogous to (5a*S,6a*R)-6,6-difluoro-3-(5-fluoro-1H-pyrazolo[3,4-b]pyridin-4-yl)-2-(5-fluoropyridin-2-yl)-5,5a,6,6a-tetrahydro-4H-cyclopropa[e]pyrazolo[1,5-a]pyridine (Example 358) except using 5-fluoro-6-methyl-1-(tetrahydro-2H-pyran-2-yl)-4-(tributylstannyl)-1H-pyrazolo[3,4-b]pyridine (Intermediate 237) instead of 5-fluoro-4-(tributylstannyl)-1-((2-(trimethylsilyl)ethoxy)methyl)-1H-pyrazolo[3,4-b]pyridine (Intermediate 235), (4aS,5aS)-3-Bromo-2-(5-fluoropyridin-2-yl)-4,4a,5,5a-tetrahydrocyclopropa[4,5]pyrrolo[1,2-b]pyrazole (Intermediate 48) instead of (5a*S,6a*R)-6,6-difluoro-3-bromo-2-(5-fluoropyridin-2-yl)-5, 5a,6,6a-tetrahydro-4H-cyclopropa[e]pyrazolo[1,5-a]pyridine (Intermediate 182), and 4N HCl in dioxane instead of TFA. MS (ESI): mass calcd. for C₁₉H₁₄F₂N₆ 364.1; m/z found 365.2 [M+H]⁺. ¹H NMR (400 MHz, CDCl₃): δ 10.72 (s, 1H), 8.17 (d, J=2.8 Hz, 1H), 7.81-7.64 (m, 1H), 7.39-7.29 (m, 2H), 4.24-4.06 (m, 1H), 3.25 (dd, J=17.3, 6.6 Hz, 1H), 2.99 (d, J=17.2 Hz, 1H), 2.66 (d, J=3.6 Hz, 3H), 2.38-2.21 (m, 1H), 1.32-1.15 (m, 1H), 0.75-0.58 (m, 1H).

### Example 274: (4aS,5aS)-3-(5-Fluoro-3,6-dimethyl-1H-pyrazolo[3,4-b]pyridin-4-yl)-2-(5-fluoropyridin-2-yl)-4,4a,5,5a-tetrahydrocyclopropa[4,5]pyrrolo[1,2-b]pyrazole

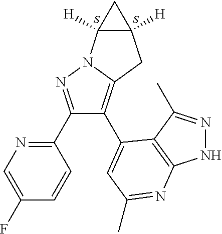

The title compound was prepared in a manner analogous to Example 358, except using and (4aS,5aS)-3-bromo-2-(5-fluoropyridin-2-yl)-4,4a,5,5a-tetrahydrocyclopropa[4,5]pyrrolo[1,2-b]pyrazole (Intermediate 48) instead of (5a*S,6a*R)-6,6-difluoro-3-bromo-2-(5-fluoropyridin-2-yl)-5,5a,6,6a-tetrahydro-4H-cyclopropa[e]pyrazolo[1,5-a]pyridine (Intermediate 182) and 5-fluoro-3,6-dimethyl-4-(tributylstannyl)-1-((2-(trimethylsilyl)ethoxy)methyl)-1H-pyrazolo[3,4-b]pyridine (Intermediate 236) instead of 5-fluoro-4-(tributylstannyl)-1-((2-(trimethylsilyl)ethoxy)methyl)-1H-pyrazolo[3,4-b]pyridine (Intermediate 235). MS (ESI): mass calcd. for C₂₀H₁₆F₂N₆ 378.1; m/z found 379.2 [M+H]⁺. ¹H NMR (400 MHz, CDCl₃): δ 8.53 (s, 1H), 7.99 (d, J=2.8 Hz, 1H), 7.97-7.87 (m, 1H), 7.46-7.34 (m, 1H), 4.28-4.16 (m, 1H), 3.30-3.02 (m, 1H), 3.02-2.81 (m, 1H), 2.75 (t, J=3.0 Hz, 3H), 2.36-2.25 (m, 1H), 2.02 (d, J=17.1 Hz, 3H), 1.43-1.24 (m, 1H), 0.76-0.56 (m, 1H).

### Example 275: (Racemic) 2-(4-Fluorophenyl)-3-(1H-pyrazolo[3,4-b]pyridin-4-yl)-3b,4,4a,5-tetrahydrocyclopropa[3,4]pyrrolo[1,2-b]pyrazole

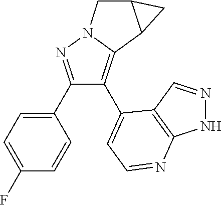 [SMILES: Fc1ccc(-c2nn3c(c2-c2ccnc4[nH]ncc24)C2CC2C3)cc1]

The title compound was prepared in a manner analogous to Example 280, except using 4-(4,4,5,5-tetramethyl-1,3,2-dioxaborolan-2-yl)-1-((2-(trimethylsilyl)ethoxy)methyl)-1H-pyrazolo[3,4-b]pyridine (Intermediate 23) instead of 5-fluoro-4-(4,4,5,5-tetramethyl-1,3,2-dioxaborolan-2-yl)-1-((2-(trimethylsilyl)ethoxy)methyl)-1H-pyrazolo[3,4-b]pyridine (Intermediate 222) and racemic (3bS,4aR)-3-bromo-2-(4-fluorophenyl)-3b,4,4a,5-tetrahydrocyclopropa[3,4]pyrrolo[1,2-b]pyrazole (Intermediate 218) instead of 3-bromo-6,6-difluoro-2-(4-fluorophenyl)-4,5,6,7-tetrahydropyrazolo[1,5-a]pyridine (Intermediate 52). MS (ESI): mass calcd. for C₁₉H₁₄FN₅ 331.1; m/z found 332.1 [M+H]⁺. ¹H NMR (400 MHz, CDCl₃): δ 12.04 (s, 1H), 8.51 (d, J=4.9 Hz, 1H), 7.84 (s, 1H), 7.44-7.34 (m, 2H), 7.02-6.92 (m, 3H), 4.48 (dd, J=11.8, 5.7 Hz, 1H), 4.40-4.19 (m, 1H), 2.56-2.37 (m, 2H), 1.52-1.38 (m, 1H), 1.00-0.84 (m, 1H).

### Example 276: (3b*R,4a*S)-2-(4-Fluorophenyl)-3-(6-methyl-1H-pyrazolo[3,4-b]pyridin-4-yl)-3b,4,4a,5-tetrahydrocyclopropa[3,4]pyrrolo[1,2-b]pyrazole

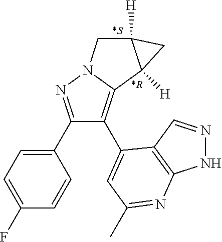 [SMILES: Cc1cc(-c2c(-c3ccc(F)cc3)nn3c2[C@@H]2C[C@@H]2C3)c2cn[nH]c2n1]

The title compound was prepared in a manner analogous to Example 1, Steps A-B, except using racemic (3bS,4aR)-3-bromo-2-(4-fluorophenyl)-3b,4,4a,5-tetrahydrocyclopropa[3,4]pyrrolo[1,2-b]pyrazole (Intermediate 163) instead of 3-bromo-2-(5-fluoropyridin-2-yl)-6,7-dihydro-4H-pyrazolo[5,1-c][1,4]oxazine (Intermediate 37), 6-methyl-4-(4,4,5,5-tetramethyl-1,3,2-dioxaborolan-2-yl)-1-((2-(trimethylsilyl)ethoxy)methyl)-1H-pyrazolo[3,4-b]pyridine (Intermediate 26) instead of 4-(4,4,5,5-tetramethyl-1,3,2-dioxaborolan-2-yl)-1-((2-(trimethylsilyl)ethoxy)methyl)-1H-pyrrolo[2,3-b]pyridine (Intermediate 21), and Pd(dppf)₂C₁₂ instead of CataCXium® A Pd G3 in Step A. The title compound and (3b*S,4a*R)-2-(4-fluorophenyl)-3-(6-methyl-1H-pyrazolo[3,4-b]pyridin-4-yl)-3b,4,4a,5-tetrahydrocyclopropa[3,4]pyrrolo[1,2-b]pyrazole (Example 277) were isolated by SFC (DAICEL CHIRALPAK AD-H 250 mm×30 mm, 5 μm (isocratic elution: EtOH (containing 0.1% of 25% aq. NH₃): supercritical CO₂, 45%: 55% to 45%: 55% (v/v))). MS (ESI): mass calcd. for C₂₀H₁₆FN₅ 345.1; m/z found 346.1 [M+H]⁺. ¹H NMR (400 MHz, DMSO-d₆) δ 13.37 (s, 1H), 7.42 (s, 1H), 7.36-7.29 (m, 2H), 7.15-7.06 (m, 2H), 6.93 (s, 1H), 4.47-4.37 (m, 1H), 4.29-4.18 (m, 1H), 2.53 (s, 3H), 2.49-2.40 (m, 2H), 1.40-1.28 (m, 1H), 0.81-0.72 (m, 1H).

### Example 277: (3b*S,4a*R)-2-(4-Fluorophenyl)-3-(6-methyl-1H-pyrazolo[3,4-b]pyridin-4-yl)-3b,4,4a,5-tetrahydrocyclopropa[3,4]pyrrolo[1,2-b]pyrazole

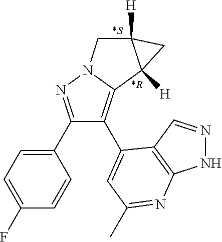 [SMILES: Cc1cc(-c2c(-c3ccc(F)cc3)nn3c2[C@H]2C[C@H]2C3)c2cn[nH]c2n1]

The title compound was isolated from chiral purification of Example 277. MS (ESI): mass calcd. for C₂₀H₁₆FN₅ 345.1; m/z found 346.0 [M+H]⁺. ¹H NMR (400 MHz, DMSO-d₆) δ 13.37 (s, 1H), 7.42 (s, 1H), 7.36-7.29 (m, 2H), 7.14-7.07 (m, 2H), 6.93 (s, 1H), 4.46-4.37 (m, 1H), 4.28-4.21 (m, 1H), 2.53 (s, 3H), 2.49-2.30 (m, 2H), 1.34 (s, 1H), 0.81-0.72 (m, 1H).

### Example 278: 4-[5,5-Difluoro-2-(4-fluorophenyl)-6,7-dihydro-4H-pyrazolo[1,5-a]pyridin-3-yl]-5-fluoro-1H-pyrazolo[3,4-b]pyridine

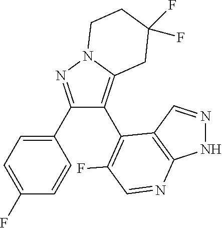 [SMILES: Fc1ccc(-c2nn3c(c2-c2c(F)cnc4[nH]ncc24)CC(F)(F)CC3)cc1]

The title compound was prepared in a manner analogous to Example 1, Steps A-B, except using 3-bromo-5, 5-difluoro-2-(4-fluorophenyl)-4,5,6,7-tetrahydropyrazolo[1,5-a]pyridine (Intermediate 54) instead of 3-bromo-2-(5-fluoropyridin-2-yl)-6,7-dihydro-4H-pyrazolo[5,1-c][1,4]oxazine (Intermediate 37) and 5-fluoro-4-(4,4,5,5-tetramethyl-1,3,2-dioxaborolan-2-yl)-1-((2-(trimethylsilyl)ethoxy)methyl)-1H-pyrazolo[3,4-b]pyridine (Intermediate 222) instead of 4-(4,4,5,5-tetramethyl-1,3,2-dioxaborolan-2-yl)-1-((2-(trimethylsilyl)ethoxy)methyl)-1H-pyrrolo[2,3-b]pyridine (Intermediate 21) in Step A. MS (ESI): mass calcd. for C₁₉H₁₃F₄N₅ 387.1; m/z found 388.1 [M+H]⁺. ¹H NMR (400 MHz, DMSO-d₆): δ 13.82 (br s, 1H), 8.60 (d, J=2.5 Hz, 1H), 7.60 (s, 1H), 7.41-7.23 (m, 2H), 7.16-6.99 (m, 2H), 4.46 (t, J=6.5 Hz, 2H), 3.51-3.35 (m, 2H), 2.83-2.62 (m, 2H).

### Example 279: 4-[6,6-Difluoro-2-(4-fluorophenyl)-5,7-dihydro-4H-pyrazolo[1,5-a]pyridin-3-yl]-6-methyl-1H-pyrazolo[3,4-d]pyrimidine

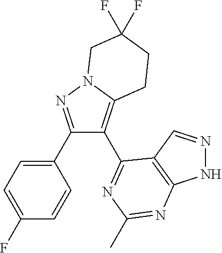 [SMILES: Cc1nc(-c2c(-c3ccc(F)cc3)nn3c2CCC(F)(F)C3)c2cn[nH]c2n1]

Step A. 4-(6,6-Difluoro-2-(4-fluorophenyl)-4,5,6,7-tetrahydropyrazolo[1,5-a]pyridin-3-yl)-6-methyl-1-((2-(trimethylsilyl)ethoxy)methyl)-1H-pyrazolo[3,4-d]pyrimidine. 4-Chloro-6-methyl-1-((2-(trimethylsilyl)ethoxy)methyl)-1H-pyrazolo[3,4-d]pyrimidine (Intermediate 212, 140 mg, 0.423 mmol) was added to a solution consisting of 3-bromo-6,6-difluoro-2-(4-fluorophenyl)-4,5,6,7-tetrahydropyrazolo[1,5-a]pyridine (Intermediate 52, 126 mg, 0.422 mmol), B₂Pin₂ (154 mg, 0.606 mmol), K₃PO₄ (280 mg, 1.32 mmol) and 1,4-dioxane/H₂O (10:1, 3.3 mL). The resultant mixture was sparged with N₂ for 5 minutes and then treated with Pd(t-Bu₃P)₂ (28 mg, 0.055 mmol). The resultant mixture was sparged with N₂ for another 5 minutes and heated at 80° C. for 16 hours. The reaction mixture was cooled to room-temperature. The mixture was poured into water (20 mL) and extracted with ethyl acetate (20 mL×3). The combined organic extracts were washed with brine (20 mL), dried over anhydrous Na₂SO₄, filtered, and concentrated to dryness under reduced pressure. The resulting residue was purified by preparative HPLC using a Welch Xtimate C18 150×25 mm×5 μm column (eluent: 70% to 100% (v/v) CH₃CN and H₂O with 0.225% HCOOH) to afford pure product. The product was suspended in water (10 mL), the mixture frozen using dry ice/EtOH, and then lyophilized to dryness to afford the title compound (35 mg, 16%) as a white solid. MS (ESI): mass calcd. for C₂₅H₂₉F₃N₆OSi 514.2; m/z found 515.1 [M+H]⁺.

Step B. 4-[6,6-Difluoro-2-(4-fluorophenyl)-5,7-dihydro-4H-pyrazolo[1,5-a]pyridin-3-yl]-6-methyl-1H-pyrazolo[3,4-d]pyrimidine. 4-(6,6-Difluoro-2-(4-fluorophenyl)-4,5,6,7-tetrahydropyrazolo[1,5-a]pyridin-3-yl)-6-methyl-1-((2-(trimethylsilyl)ethoxy)methyl)-1H-pyrazolo[3,4-d]pyrimidine (35 mg, 0.056 mmol), TFA (2 mL), and dichloromethane (1 mL) were added to a 25 mL round-bottomed flask. The resulting solution was stirred at room-temperature for 16 hours. The reaction solution was concentrated to dryness under reduced pressure to afford the title compound, which was diluted with methanol (5 mL), and treated with 7 M NH₃ in methanol (2 mL). The resultant solution was stirred at room-temperature for 30 min. The reaction solution was concentrated to dryness under reduced pressure. The resulting residue was purified by preparative HPLC using a Welch Xtimate C18 150×25 mm×5 μm column (eluent: 35% to 65% (v/v) CH₃CN and H₂O with 0.2% HCOOH) to afford the title compound (12.8 mg, 60%) as a light yellow solid. MS (ESI): mass calcd. for C₁₉H₁₅F₃N₆ 384.1; m/z found 385.0 [M+H]⁺. ¹H NMR (400 MHz, CDCl₃): δ 11.06 (br s, 1H), 7.48-7.36 (m, 2H), 7.09-6.90 (m, 3H), 4.60 (t, J=12.2 Hz, 2H), 3.39 (t, J=6.8 Hz, 2H), 2.88 (s, 3H), 2.43 (tt, J=6.9, 13.4 Hz, 2H).

### Example 280: 4-(6,6-Difluoro-2-(4-fluorophenyl)-4,5,6,7-tetrahydropyrazolo[1,5-a]pyridin-3-yl)-5-fluoro-1H-pyrazolo[3,4-b]pyridine

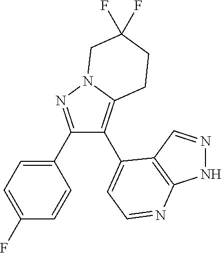 [SMILES: Fc1ccc(-c2nn3c(c2-c2ccnc4[nH]ncc24)CCC(F)(F)C3)cc1]

The title compound was prepared in a manner analogous to Example 221, Steps B-C, except using 5-fluoro-4-(4,4,5,5-tetramethyl-1,3,2-dioxaborolan-2-yl)-1-((2-(trimethylsilyl)ethoxy)methyl)-1H-pyrazolo[3,4-b]pyridine (Intermediate 222) instead of 2-(2-(5-fluoropyridin-2-yl)-5, 6-dihydro-4H-pyrrolo[1,2-b]pyrazol-3-yl)-2-hydroxy-4,4,5,5-tetramethyl-1,3,2-dioxaborolan-2-uide (product from Step A); 3-bromo-6,6-difluoro-2-(4-fluorophenyl)-4,5,6,7-tetrahydropyrazolo[1,5-a]pyridine (Intermediate 52) instead of 5-fluoro-4-iodo-1-((2-(trimethylsilyl)ethoxy)methyl)-1H-pyrazolo[3,4-b]pyridine (Intermediate 204); CatacXium Pd G4® instead of CataCXium® A Pd G3; and heating for 2 hrs instead of 16 hrs in Step B. MS (ESI): mass calcd. for C₁₉H₁₃F₄N₅ 387.1; m/z found, 388.1 [M+H]⁺. ¹H NMR (400 MHz, CDCl₃): δ 10.46 (s, 1H), 8.52 (d, J=2.6 Hz, 1H), 7.43 (s, 1H), 7.38-7.29 (m, 2H), 6.91 (t, J=8.7 Hz, 2H), 4.62 (q, J=12.2 Hz, 2H), 3.16-3.01 (m, 1H), 2.90-2.77 (m, 1H), 2.55-2.26 (m, 2H).

### Example 281: 4-[2-(5-Fluoro-2-pyridyl)-6,6-dimethyl-5,7-dihydro-4H-pyrazolo[1,5-a]pyridin-3-yl]pyridin-2-amine

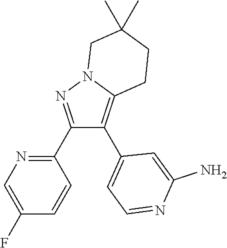 [SMILES: CC1(C)CCc2c(-c3ccnc(N)c3)c(-c3ccc(F)cn3)nn2C1]

The title compound was prepared in a manner analogous to Example 1, Step A, using (2-aminopyridin-4-yl)boronic acid instead of 4-(4,4,5,5-tetramethyl-1,3,2-dioxaborolan-2-yl)-1-((2-(trimethylsilyl)ethoxy)methyl)-1H-pyrrolo[2,3-b]pyridine (Intermediate 21) and 3-bromo-2-(5-fluoropyridin-2-yl)-6,6-dimethyl-4,5,6,7-tetrahydropyrazolo[1,5-a]pyridine (Intermediate 57) instead of 3-bromo-2-(5-fluoropyridin-2-yl)-6,7-dihydro-4H-pyrazolo[5,1-c][1,4]oxazine (Intermediate 37). MS (ESI): mass calcd. for C₁₉H₂₀FN₅ 337.2; m/z found 338.1 [M+H]⁺. ¹H NMR (400 MHz, DMSO-d₆): δ 8.46 (d, J=2.5 Hz, 1H), 7.78-7.66 (m, 3H), 6.33-6.24 (m, 2H), 5.76 (s, 2H), 3.87 (s, 2H), 2.79 (m, 2H), 1.66 (m, 2H), 1.07 (s, 6H).

### Example 282: 5-Fluoro-4-[2-(5-fluoro-2-pyridyl)-6,6-dimethyl-5,7-dihydro-4H-pyrazolo[1,5-a]pyridin-3-yl]-1H-pyrazolo[3,4-b]pyridine

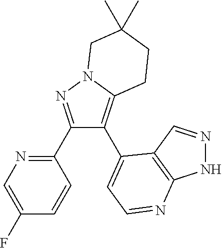 [SMILES: CC1(C)CCc2c(-c3ccnc4[nH]ncc34)c(-c3ccc(F)cn3)nn2C1]

The title compound was prepared in a manner analogous to Example 1, Steps A-B, using 3-bromo-2-(5-fluoropyridin-2-yl)-6,6-dimethyl-4,5,6,7-tetrahydropyrazolo[1,5-a]pyridine (Intermediate 57) instead of 3-bromo-2-(5-fluoropyridin-2-yl)-6,7-dihydro-4H-pyrazolo[5,1-c][1,4]oxazine (Intermediate 37) and 5-fluoro-4-(4,4,5,5-tetramethyl-1,3,2-dioxaborolan-2-yl)-1-((2-(trimethylsilyl)ethoxy)methyl)-1H-pyrazolo[3,4-b]pyridine (Intermediate 222) instead of 4-(4,4,5,5-tetramethyl-1,3,2-dioxaborolan-2-yl)-1-((2-(trimethylsilyl)ethoxy)methyl)-1H-pyrrolo[2,3-b]pyridine (Intermediate 21) in Step A. MS (ESI): mass calcd. for C₂₀H₁₈F₂N₆ 380.2; m/z found 381.2 [M+H]⁺. ¹H NMR (400 MHz, DMSO-d₆): δ 8.50 (d, J=2.5 Hz, 1H), 8.17 (d, J=3.0 Hz, 1H), 7.92 (m, 1H), 7.73 (m, 1H), 7.67 (s, 1H), 3.97 (s, 2H), 2.79-2.54 (m, 2H), 1.77-1.57 (m, 2H), 1.12 (s, 3H), 1.08 (s, 3H). N—H proton not observed.

### Example 283: 4-[2-(5-Fluoro-2-pyridyl)-6,6-bis(methyl-d₃)-5,7-dihydro-4H-pyrazolo[1,5-a]pyridin-3-yl]-1H-pyrazolo[3,4-b]pyridine

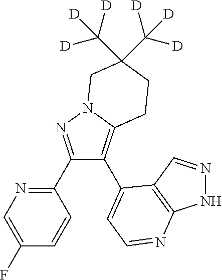 [SMILES: [2H]C([2H])([2H])C1(C([2H])([2H])[2H])CCc2c(-c3ccnc4[nH]ncc34)c(-c3ccc(F)cn3)nn2C1]

The title compound was prepared in a manner analogous to Example 1, Steps A-B, except using 3-bromo-2-(5-fluoropyridin-2-yl)-6,6-bis(methyl-d₃)-4,5,6,7-tetrahydropyrazolo[1,5-a]pyridine (Intermediate 168) instead of 3-bromo-2-(5-fluoropyridin-2-yl)-6,7-dihydro-4H-pyrazolo[5,1-c][1,4]oxazine (Intermediate 37) and 4-(4,4,5,5-tetramethyl-1,3,2-dioxaborolan-2-yl)-1-((2-(trimethylsilyl)ethoxy)methyl)-1H-pyrazolo[3,4-b]pyridine (Intermediate 23) instead of 4-(4,4,5,5-tetramethyl-1,3,2-dioxaborolan-2-yl)-1-((2-(trimethylsilyl)ethoxy)methyl)-1H-pyrrolo[2,3-b]pyridine (Intermediate 21) in Step A. MS (ESI): mass calcd. for C₂₀H₁₃D₆FN₆ 368.2; m/z found 369.5 [M+H]⁺. ¹H NMR (400 MHz, DMSO-d₆): δ 13.48 (s, 1H), 8.45 (d, J=3.0 Hz, 1H), 8.20 (s, 1H), 7.95-7.62 (m, 2H), 7.33-7.30 (m, 1H), 7.12-7.09 (m, 1H), 4.01-3.89 (m, 2H), 2.88-2.75 (m, 2H), 1.74-1.59 (m, 2H).

### Example 284: (*S)-6-(Fluoromethyl)-2-(5-fluoro-2-pyridyl)-6-methyl-3-(4-pyridyl)-5,7-dihydro-4H-pyrazolo[1,5-a]pyridine

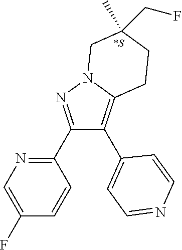 [SMILES: C[C@]1(CF)CCc2c(-c3ccncc3)c(-c3ccc(F)cn3)nn2C1]

The title compound was prepared in a manner analogous to Example 1, Step A, using pyridin-4-ylboronic acid instead of 4-(4,4,5,5-tetramethyl-1,3,2-dioxaborolan-2-yl)-1-((2-(trimethylsilyl)ethoxy)methyl)-1H-pyrrolo[2,3-b]pyridine (Intermediate 21) and 3-bromo-6-(fluoromethyl)-2-(5-fluoropyridin-2-yl)-6-methyl-4,5,6,7-tetrahydropyrazolo[1,5-a]pyridine (Intermediate 60) instead of 3-bromo-2-(5-fluoropyridin-2-yl)-6,7-dihydro-4H-pyrazolo[5,1-c][1,4]oxazine (Intermediate 37). The title compound and (*R)-6-(fluoromethyl)-2-(5-fluoro-2-pyridyl)-6-methyl-3-(4-pyridyl)-5,7-dihydro-4H-pyrazolo[1,5-a]pyridine were resolved using chiral SFC (CHIRALPAK IC 250 mm×30 mm, 10 μm (isocratic elution: EtOH (containing 0.1% of 25% aq. NH₃): supercritical CO₂, 40%: 60% to 40%: 60% (v/v)). MS (ESI): mass calcd. for C₁₉H₁₈F₂N₄ 340.2; m/z found 341.1 [M+H]⁺. ¹H NMR (400 MHz, DMSO-d₆): δ 8.51-8.35 (m, 3H), 7.87-7.70 (m, 2H), 7.27-7.16 (m, 2H), 4.55-4.43 (m, 1H), 4.40-4.30 (m, 1H), 4.18-4.06 (m, 1H), 4.03-3.90 (m, 1H), 2.87 (t, J=6.6 Hz, 2H), 1.89-1.60 (m, 2H), 1.11 (s, 3H).

### Example 285: (*R)-6-(Fluoromethyl)-2-(5-fluoro-2-pyridyl)-6-methyl-3-(4-pyridyl)-5,7-dihydro-4H-pyrazolo[1,5-a]pyridine

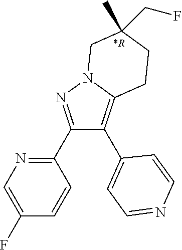 [SMILES: C[C@@]1(CF)CCc2c(-c3ccncc3)c(-c3ccc(F)cn3)nn2C1]

The title compound was isolated from chiral purification of Example 284. MS (ESI): mass calcd. for C₁₉H₁₈F₂N₄ 340.2; m/z found 341.1 [M+H]⁺. ¹H NMR (400 MHz, DMSO-d₆): δ 8.54-8.34 (m, 3H), 7.90-7.68 (m, 2H), 7.28-7.15 (m, 2H), 4.56-4.44 (m, 1H), 4.40-4.29 (m, 1H), 4.17-4.04 (m, 1H), 4.01-3.89 (m, 1H), 2.87 (t, J=6.6 Hz, 2H), 1.88-1.62 (m, 2H), 1.11 (s, 3H).

### Example 286: (*S)-4-[6-(Fluoromethyl)-2-(5-fluoro-2-pyridyl)-6-methyl-5,7-dihydro-4H-pyrazolo[1,5-a]pyridin-3-yl]pyridin-2-amine

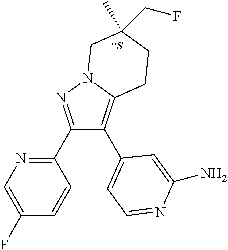 [SMILES: C[C@]1(CF)CCc2c(-c3ccnc(N)c3)c(-c3ccc(F)cn3)nn2C1]

The title compound was prepared in a manner analogous to Example 1, Step A, using (2-aminopyridin-4-yl)boronic acid instead of 4-(4,4,5,5-tetramethyl-1,3,2-dioxaborolan-2-yl)-1-((2-(trimethylsilyl)ethoxy)methyl)-1H-pyrrolo[2,3-b]pyridine (Intermediate 21) and 3-bromo-6-(fluoromethyl)-2-(5-fluoropyridin-2-yl)-6-methyl-4,5,6,7-tetrahydropyrazolo[1,5-a]pyridine (Intermediate 60) instead of 3-bromo-2-(5-fluoropyridin-2-yl)-6,7-dihydro-4H-pyrazolo[5,1-c][1,4]oxazine (Intermediate 37). The title compound and (*R)-4-[6-(fluoromethyl)-2-(5-fluoro-2-pyridyl)-6-methyl-5,7-dihydro-4H-pyrazolo[1,5-a]pyridin-3-yl]pyridin-2-amine (Example 287) were resolved using chiral SFC (DAICEL CHIRALPAK IG 250 mm×30 mm, 10 μm (isocratic elution: MeOH (containing 0.1% of 25% aq. NH₃): supercritical CO₂, 50%: 50% to 50%: 50% (v/v))). MS (ESI): mass calcd. for C₁₉H₁₉F₂N₅ 355.2; m/z found 356.1 [M+H]⁺. ¹H NMR (400 MHz, DMSO-d₆): δ 8.47 (d, J=2.8 Hz, 1H), 7.83-7.62 (m, 3H), 6.41-6.26 (m, 2H), 5.92 (s, 2H), 4.47 (s, 1H), 4.35 (s, 1H), 4.14-4.03 (m, 1H), 4.01-3.89 (m, 1H), 2.83 (t, J=6.5 Hz, 2H), 1.88-1.65 (m, 2H), 1.10 (s, 3H).

### Example 287: (*R)-4-[6-(Fluoromethyl)-2-(5-fluoro-2-pyridyl)-6-methyl-5,7-dihydro-4H-pyrazolo[1,5-a]pyridin-3-yl]pyridin-2-amine

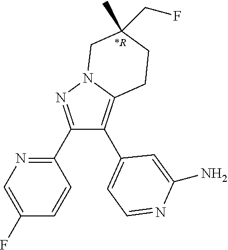 [SMILES: C[C@@]1(CF)CCc2c(-c3ccnc(N)c3)c(-c3ccc(F)cn3)nn2C1]

The title compound was isolated from chiral purification of Example 286. MS (ESI): mass calcd. for C₁₉H₁₉F₂N₅ 355.2; m/z found 356.1 [M+H]⁺. ¹H NMR (400 MHz, DMSO-d₆): δ 8.46 (d, J=2.5 Hz, 1H), 7.80-7.66 (m, 3H), 6.37-6.25 (m, 2H), 5.87 (s, 2H), 4.47 (s, 1H), 4.35 (s, 1H), 4.16-4.04 (m, 1H), 4.01-3.87 (m, 1H), 2.83 (t, J=6.4 Hz, 2H), 1.86-1.67 (m, 2H), 1.10 (s, 3H).

### Example 288: (Racemic) 3-(2,5-Difluoro-4-pyridyl)-6-(fluoromethyl)-2-(5-fluoro-2-pyridyl)-6-methyl-5,7-dihydro-4H-pyrazolo[1,5-a]pyridine

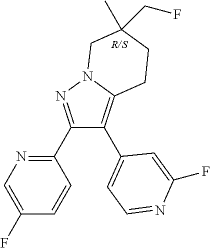 [SMILES: CC1(CF)CCc2c(-c3ccnc(F)c3)c(-c3ccc(F)cn3)nn2C1]

The title compound was prepared in a manner analogous to Example 1, Step A, using (2,5-difluoropyridin-4-yl)boronic acid instead of 4-(4,4,5,5-tetramethyl-1,3,2-dioxaborolan-2-yl)-1-((2-(trimethylsilyl)ethoxy)methyl)-1H-pyrrolo[2,3-b]pyridine (Intermediate 21), 3-bromo-6-(fluoromethyl)-2-(5-fluoropyridin-2-yl)-6-methyl-4,5,6,7-tetrahydropyrazolo[1,5-a]pyridine (Intermediate 60) instead of 3-bromo-2-(5-fluoropyridin-2-yl)-6,7-dihydro-4H-pyrazolo[5,1-c][1,4]oxazine (Intermediate 37), and Pd(dppf)Cl₂ instead of CataCXium® A Pd G3. MS (ESI): mass calcd. for C₁₉H₁₆F₄N₄ 376.1; m/z found 377.0 [M+H]⁺. ¹H NMR (400 MHz, DMSO-d₆): δ 8.35 (d, J=2.8 Hz, 1H), 8.13 (s, 1H), 7.98-7.87 (m, 1H), 7.83-7.71 (m, 1H), 7.38-7.37 (m, 1H), 4.55-4.45 (m, 1H), 4.41-4.28 (m, 1H), 4.18-4.08 (m, 1H), 4.05-3.90 (m, 1H), 2.86-2.83 (m, 2H), 1.86-1.65 (m, 2H), 1.11 (s, 3H).

### Example 289: (*R)-5-[6-(Fluoromethyl)-2-(5-fluoro-2-pyridyl)-6-methyl-5,7-dihydro-4H-pyrazolo[1,5-a]pyridin-3-yl]pyrazolo[1,5-a]pyridine

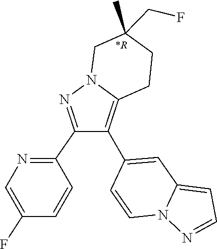 [SMILES: C[C@@]1(CF)CCc2c(-c3ccn4nccc4c3)c(-c3ccc(F)cn3)nn2C1]

The title compound was prepared in a manner analogous to Example 1, Step A, using 5-(4,4,5,5-tetramethyl-1,3,2-dioxaborolan-2-yl)pyrazolo[1,5-a]pyridine (Intermediate 224) instead of 4-(4,4,5,5-tetramethyl-1,3,2-dioxaborolan-2-yl)-1-((2-(trimethylsilyl)ethoxy)methyl)-1H-pyrrolo[2,3-b]pyridine (Intermediate 21) and 3-bromo-6-(fluoromethyl)-2-(5-fluoropyridin-2-yl)-6-methyl-4,5,6,7-tetrahydropyrazolo[1,5-a]pyridine (Intermediate 60) instead of 3-bromo-2-(5-fluoropyridin-2-yl)-6,7-dihydro-4H-pyrazolo[5,1-c][1,4]oxazine (Intermediate 37). The title compound and (*S)-5-[6-(fluoromethyl)-2-(5-fluoro-2-pyridyl)-6-methyl-5,7-dihydro-4H-pyrazolo[1,5-a]pyridin-3-yl]pyrazolo[1,5-a]pyridine (Example 290) were resolved using chiral SFC (over DAICEL CHIRALPAK IC 250 mm×30 mm, 10 μm (isocratic elution: EtOH (containing 0.1% of 25% aq. NH₃): supercritical CO₂, 50%: 50% to 50%: 50% (v/v))). MS (ESI): mass calcd. for C₂₁H₁₉F₂N₅ 379.2; m/z found 380.2 [M+H]⁺. ¹H NMR (400 MHz, DMSO-d₆): δ 8.52 (d, J=7.1 Hz, 1H), 8.42 (d, J=2.9 Hz, 1H), 7.96 (d, J=2.2 Hz, 1H), 7.87-7.79 (m, 1H), 7.78-7.70 (m, 1H), 7.60 (d, J=0.7 Hz, 1H), 6.60-6.58 (m, 1H), 6.53 (d, J=1.5 Hz, 1H), 4.55-4.44 (m, 1H), 4.42-4.29 (m, 1H), 4.17-4.05 (m, 1H), 4.03-3.93 (m, 1H), 2.88 (t, J=6.5 Hz, 2H), 1.85-1.79 (m, 1H), 1.76-1.62 (m, 1H), 1.13 (s, 3H).

### Example 290: (*S)-5-[6-(Fluoromethyl)-2-(5-fluoro-2-pyridyl)-6-methyl-5,7-dihydro-4H-pyrazolo[1,5-a]pyridin-3-yl]pyrazolo[1,5-a]pyridine

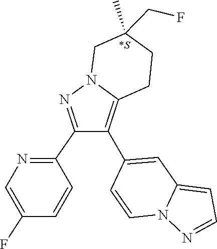 [SMILES: C[C@]1(CF)CCc2c(-c3ccn4nccc4c3)c(-c3ccc(F)cn3)nn2C1]

The title compound was isolated from chiral SFC purification of Example 289. MS (ESI): mass calcd. for C₂₁H₁₉F₂N₅ 379.2; m/z found 380.2 [M+H]⁺. ¹H NMR (400 MHz, DMSO-d₆): δ 8.52 (d, J=7.3 Hz, 1H), 8.42 (d, J=2.9 Hz, 1H), 7.96 (d, J=2.0 Hz, 1H), 7.88-7.80 (m, 1H), 7.79-7.70 (m, 1H), 7.60 (s, 1H), 6.60-6.58 (m, 1H), 6.53 (d, J=1.8 Hz, 1H), 4.55-4.45 (m, 1H), 4.42-4.32 (m, 1H), 4.17-4.05 (m, 1H), 4.02-3.89 (m, 1H), 2.88 (t, J=6.5 Hz, 2H), 1.85-1.78 (m, 1H), 1.76-1.64 (m, 1H), 1.12 (s, 3H).

### Example 291: (*R)-3-Chloro-4-(6-(fluoromethyl)-2-(5-fluoropyridin-2-yl)-6-methyl-4,5,6,7-tetrahydropyrazolo[1,5-a]pyridin-3-yl)-1H-pyrazolo[3,4-b]pyridine

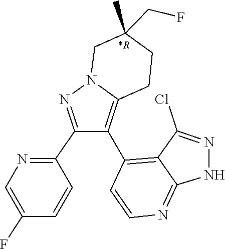 [SMILES: C[C@@]1(CF)CCc2c(-c3ccnc4[nH]nc(Cl)c34)c(-c3ccc(F)cn3)nn2C1]

The title compound was prepared in a manner analogous to Example 224, except using (R)-4-(6-(fluoromethyl)-2-(5-fluoropyridin-2-yl)-6-methyl-4,5,6,7-tetrahydropyrazolo[1,5-a]pyridin-3-yl)-1H-pyrazolo[3,4-b]pyridine (Example 51) instead of Example 9. MS (ESI): mass calcd. for C₂₀H₁₇ClF₂N₆ 414.1; m/z found 415.1 [M+H]⁺. ¹H NMR (400 MHz, CD₃OD): δ δ 8.54 (d, J=4.7 Hz, 1H), 8.12 (dd, J=3.1, 0.9 Hz, 1H), 7.80-7.66 (m, 1H), 7.55 (td, J=8.6, 2.9 Hz, 1H), 7.13 (dd, J=4.8, 0.9 Hz, 1H), 4.55-4.41 (m, 1H), 4.39-4.31 (m, 1H), 4.24 (dd, J=13.0, 5.5 Hz, 1H), 4.08 (dd, J=13.0, 5.4 Hz, 1H), 2.92-2.75 (m, 1H), 2.75-2.57 (m, 1H), 1.93 (q, J=7.1, 6.6 Hz, 1H), 1.77 (dt, J=14.2, 7.1 Hz, 1H), 1.20 (dd, J=10.1, 1.6 Hz, 3H). N—H proton not observed.

### Example 292: (*R)-5-Fluoro-4-[6-(fluoromethyl)-2-(5-fluoro-2-pyridyl)-6-methyl-5,7-dihydro-4H-pyrazolo[1,5-a]pyridin-3-yl]-1H-pyrazolo[3,4-b]pyridine

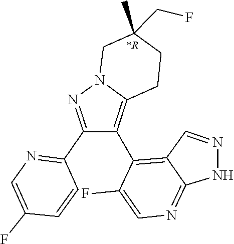 [SMILES: C[C@@]1(CF)CCc2c(-c3c(F)cnc4[nH]ncc34)c(-c3ccc(F)cn3)nn2C1]

A mixture of title compound was prepared in a manner analogous to Example 1, Steps A-B, except using 3-bromo-6-(fluoromethyl)-2-(5-fluoropyridin-2-yl)-6-methyl-4,5,6,7-tetrahydropyrazolo[1,5-a]pyridine (Intermediate 60) instead of 3-bromo-2-(5-fluoropyridin-2-yl)-6,7-dihydro-4H-pyrazolo[5,1-c][1,4]oxazine (Intermediate 37) and 5-fluoro-4-(4,4,5,5-tetramethyl-1,3,2-dioxaborolan-2-yl)-1-((2-(trimethylsilyl)ethoxy)methyl)-1H-pyrazolo[3,4-b]pyridine (Intermediate 222) instead of 4-(4,4,5,5-tetramethyl-1,3,2-dioxaborolan-2-yl)-1-((2-(trimethylsilyl)ethoxy)methyl)-1H-pyrrolo[2,3-b]pyridine (Intermediate 21) in Step A. Purification (SFC, DAICEL CHIRALPAK AD 250 mm×30 mm, 10 μm (isocratic elution: IPA (containing 0.1% of 25% aq. NH₃): supercritical CO₂, 30%: 70% to 30%: 70% (v/v)) afforded the title compound: MS (ESI): mass calcd. for C₂₀H₁₇F₃N₆ 398.1; m/z found 399.1 [M+H]⁺; ¹H NMR (400 MHz, DMSO-d₆): δ 13.71 (s, 1H), 8.56-8.41 (m, 1H), 8.19 (s, 1H), 7.95-7.91 (m, 1H), 7.81-7.64 (m, 2H), 4.68-4.29 (m, 2H), 4.25-4.12 (m, 1H), 4.10-4.00 (m, 1H), 2.77-2.60 (m, 2H), 1.97-1.62 (m, 2H), 1.20-1.09 (m, 3H); and (*S)-5-Fluoro-4-[6-(fluoromethyl)-2-(5-fluoro-2-pyridyl)-6-methyl-5,7-dihydro-4H-pyrazolo[1,5-a]pyridin-3-yl]-1H-pyrazolo[3,4-b]pyridine (Example 293).

### Example 293: (*S)-5-Fluoro-4-[6-(fluoromethyl)-2-(5-fluoro-2-pyridyl)-6-methyl-5,7-dihydro-4H-pyrazolo[1,5-a]pyridin-3-yl]-1H-pyrazolo[3,4-b]pyridine

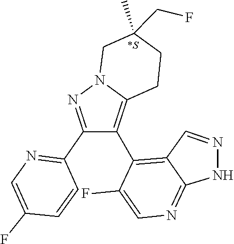 [SMILES: C[C@]1(CF)CCc2c(-c3c(F)cnc4[nH]ncc34)c(-c3ccc(F)cn3)nn2C1]

The title compound was isolated from chiral SFC purification of Example 292. MS (ESI): mass calcd. for C₂₀H₁₇F₃N₆ 398.1; m/z found 399.1 [M+H]⁺. ¹H NMR (400 MHz, DMSO-d₆): δ 13.72 (s, 1H), 8.55-8.44 (m, 1H), 8.19 (s, 1H), 7.95-7.91 (m, 1H), 7.82-7.69 (m, 2H), 4.57-4.29 (m, 2H), 4.25-4.13 (m, 1H), 4.08-4.00 (m, 1H), 2.81-2.60 (m, 2H), 1.93-1.62 (m, 2H), 1.18-1.09 (m, 3H).

### Example 294: (*R)-5-Fluoro-4-(6-(fluoromethyl-d₂)-2-(5-fluoropyridin-2-yl)-6-(methyl-d₃)-4,5,6,7-tetrahydropyrazolo[1,5-a]pyridin-3-yl)-1H-pyrazolo[3,4-b]pyridine

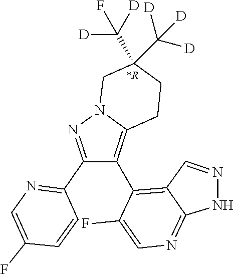 [SMILES: [2H]C([2H])([2H])[C@@]1(C([2H])([2H])F)CCc2c(-c3c(F)cnc4[nH]ncc34)c(-c3ccc(F)cn3)nn2C1]

Step A. 5-Fluoro-4-(6-(fluoromethyl-d₂)-2-(5-fluoropyridin-2-yl)-6-(methyl-d₃)-4,5,6,7-tetrahydropyrazolo[1,5-a]pyridin-3-yl)-1-(tetrahydro-2H-pyran-2-yl)-1H-pyrazolo[3,4-b]pyridine. 3-Bromo-6-(fluoromethyl-d₂)-2-(5-fluoropyridin-2-yl)-6-(methyl-d₃)-4,5,6,7-tetrahydropyrazolo[1,5-a]pyridine (Intermediate 169, 230 mg, 0.662 mmol), 5-fluoro-4-iodo-1-(tetrahydro-2H-pyran-2-yl)-1H-pyrazolo[3,4-b]pyridine (Intermediate 230, 345 mg, 0.994 mmol), B₂Pin₂ (252 mg, 0.992 mmol) and Cs₂CO₃ (648 mg, 1.99 mmol) were dissolved in 1,4-dioxane (10 mL) and H₂O (1 mL). The resultant mixture was sparged with N₂ for 5 minutes and then treated with Pd(t-Bu₃P)₂ (34.2 mg, 0.067 mmol). The resultant mixture was sparged with N₂ for another 5 minutes and then then the mixture was stirred at 100° C. for 16 hours. The resultant mixture was concentrated to dryness under reduced pressure. The resulting residue was purified by FCC (SiO₂, eluent: petroleum ether:ethyl acetate=0-75% EtOAc) to afford the crude compound (120 mg) as a white solid. The crude compound was purified by preparative HPLC using a Boston Green ODS 150×30 mm×5 μm (eluent: 55% to 85% (v/v) MeCN and H₂O 0.225% HCOOH) to afford pure product, which was used directly in the next step.

Step B. (*R)-5-Fluoro-4-(6-(fluoromethyl-d₂)-2-(5-fluoropyridin-2-yl)-6-(methyl-d₃)-4,5,6,7-tetrahydropyrazolo[1,5-a]pyridin-3-yl)-1H-pyrazolo[3,4-b]pyridine. HCl/1,4-dioxane (5 mL) was added to 5-Fluoro-4-(6-(fluoromethyl-d₂)-2-(5-fluoropyridin-2-yl)-6-(methyl-d₃)-4,5,6,7-tetrahydropyrazolo[1,5-a]pyridin-3-yl)-1-(tetrahydro-2H-pyran-2-yl)-1H-pyrazolo[3,4-b]pyridine (120 mg, 0.246 mmol). The mixture was stirred at room-temperature for 1 hour. The mixture was concentrated to dryness under reduced pressure. The resulting residue was purified by preparative HPLC using a Welch Xtimate C18 150 mm×30 mm×5 μm (eluent: 35% to 65% (v/v) MeCN and H₂O with 0.05% NH₃+10 mM NH₄HCO₃) followed by chiral SFC (DAICEL CHIRALCEL OJ (250 mm×30 mm×10 m) (isocratic elution: IPA (containing 0.1% aq. NH₃): supercritical CO₂, 30%: 70% to 30%: 70% (v/v))). MS (ESI): mass calcd. for C₂₀H₁₂D₅F₃N₆ 403.2; m/z found 404.2 [M+H]⁺. ¹H NMR (400 MHz, CDCl₃): δ 10.70 (s, 1H), 8.53-8.47 (m, 1H), 8.22-8.16 (m, 1H), 7.69-7.60 (m, 1H), 7.52 (d, J=16.6 Hz, 1H), 7.37-7.28 (m, 1H), 4.30-4.21 (m, 1H), 4.16-4.03 (m, 1H), 3.02-2.83 (m, 1H), 2.79-2.65 (m, 1H), 2.04-1.88 (m, 1H), 1.78-1.69 (m, 1H).

### Example 295: (*S)-5-Fluoro-4-(6-(fluoromethyl-d₂)-2-(5-fluoropyridin-2-yl)-6-(methyl-d₅)-4,5,6,7-tetrahydropyrazolo[1,5-a]pyridin-3-yl)-1H-pyrazolo[3,4-b]pyridine

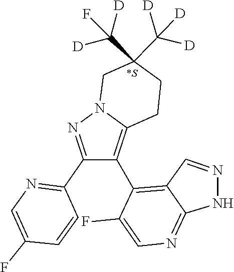 [SMILES: [2H]C([2H])([2H])[C@]1(C([2H])([2H])F)CCc2c(-c3c(F)cnc4[nH]ncc34)c(-c3ccc(F)cn3)nn2C1]

The title compound was prepared in a manner analogous to (*R)-5-fluoro-4-(6-(fluoromethyl-d₂)-2-(5-fluoropyridin-2-yl)-6-(methyl-d₃)-4,5,6,7-tetrahydropyrazolo[1,5-a]pyridin-3-yl)-1H-pyrazolo[3,4-b]pyridine (Example 294). MS (ESI): mass calcd. for C₂₀H₁₂D₅F₃N₆ 403.2; m/z found 404.1 [M+H]⁺. ¹H NMR (400 MHz, CDCl₃): δ 10.73 (s, 1H), 8.53-8.47 (m, 1H), 8.23-8.15 (m, 1H), 7.69-7.61 (m, 1H), 7.55-7.48 (m, 1H), 7.36-7.28 (m, 1H), 4.30-4.21 (m, 1H), 4.15-4.05 (m, 1H), 2.97-2.84 (m, 1H), 2.78-2.67 (m, 1H), 2.03-1.91 (m, 1H), 1.79-1.70 (m, 1H).

### Example 296: (*S)-4-[2-(5-Fluoro-2-pyridyl)-6-(methoxymethyl)-4,5,6,7-tetrahydropyrazolo[1,5-a]pyridin-3-yl]-6-methyl-1H-pyrazolo[3,4-b]pyridine

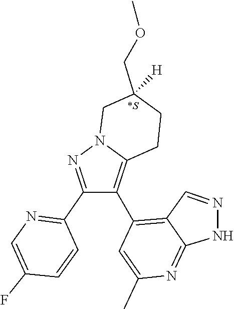 [SMILES: COC[C@H]1CCc2c(-c3cc(C)nc4[nH]ncc34)c(-c3ccc(F)cn3)nn2C1]

Title compound was prepared in a manner analogous to Example 1, Steps A-B, except using 3-bromo-2-(5-fluoropyridin-2-yl)-6-(methoxymethyl)-4,5,6,7-tetrahydropyrazolo[1,5-a]pyridine (Intermediate 165) instead of 3-bromo-2-(5-fluoropyridin-2-yl)-6,7-dihydro-4H-pyrazolo[5,1-c][1,4]oxazine (Intermediate 37) and 6-methyl-4-(4,4,5,5-tetramethyl-1,3,2-dioxaborolan-2-yl)-1-((2-(trimethylsilyl)ethoxy)methyl)-1H-pyrazolo[3,4-b]pyridine (Intermediate 26) instead of 4-(4,4,5,5-tetramethyl-1,3,2-dioxaborolan-2-yl)-1-((2-(trimethylsilyl)ethoxy)methyl)-1H-pyrrolo[2,3-b]pyridine (Intermediate 21) in Step A. Purification via chiral SFC (DAICEL CHIRALPAK AD (250 mm×30 mm, 10 um) (isocratic elution: EtOH (containing 0.1% aq. NH₃): supercritical CO₂, 40%: 60% to 40%: 60% (v/v))) afforded the title compound: MS (ESI): mass calcd. for C₂₁H₂₁FN₆O 392.2; m/z found 393.3 [M+H]⁺. ¹H NMR (400 MHz, DMSO-d₆): δ 13.24 (s, 1H), 8.22 (d, J=2.8 Hz, 1H), 7.83-7.77 (m, 1H), 7.76-7.69 (m, 1H), 7.19 (s, 1H), 6.94 (s, 1H), 4.35 (dd, J=5.1, 12.7 Hz, 1H), 3.96-3.87 (m, 1H), 3.48-3.42 (m, 2H), 3.31 (s, 3H), 2.87-2.75 (m, 2H), 2.56 (s, 3H), 2.49-2.39 (m, 1H), 2.00-1.92 (m, 1H), 1.61-1.50 (m, 1H); and (*R)-4-[2-(5-Fluoro-2-pyridyl)-6-(methoxymethyl)-4,5,6,7-tetrahydropyrazolo[1,5-a]pyridin-3-yl]-6-methyl-1H-pyrazolo[3,4-b]pyridine (Example 297).

### Example 297: (*R)-4-[2-(5-Fluoro-2-pyridyl)-6-(methoxymethyl)-4,5,6,7-tetrahydropyrazolo[1,5-a]pyridin-3-yl]-6-methyl-1H-pyrazolo[3,4-b]pyridine

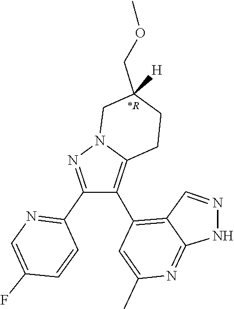 [SMILES: COC[C@@H]1CCc2c(-c3cc(C)nc4[nH]ncc34)c(-c3ccc(F)cn3)nn2C1]

The title compound was isolated from chiral SFC purification of Example 296. MS (ESI): mass calcd. for C₂₁H₂₁FN₆O 392.2; m/z found 393.3 [M+H]⁺. ¹H NMR (400 MHz, DMSO-d₆): δ 13.24 (s, 1H), 8.22 (d, J=2.8 Hz, 1H), 7.83-7.69 (m, 2H), 7.19 (s, 1H), 6.94 (s, 1H), 4.39-4.32 (m, 1H), 3.95-3.86 (m, 1H), 3.46-3.43 (m, 2H), 3.31 (s, 3H), 2.86-2.76 (m, 2H), 2.56 (s, 3H), 2.49-2.39 (m, 1H), 2.00-1.88 (m, 1H), 1.65-1.46 (m, 1H).

### Example 298: (*S)-4-[6-Fluoro-2-(5-fluoro-2-pyridyl)-6-(methoxymethyl)-5,7-dihydro-4H-pyrazolo[1,5-a]pyridin-3-yl]-6-methyl-1H-pyrrolo[2,3-b]pyridine

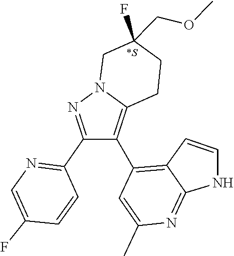 [SMILES: COC[C@]1(F)CCc2c(-c3cc(C)nc4[nH]ccc34)c(-c3ccc(F)cn3)nn2C1]

The title compound was prepared in a manner analogous to 4-[2-(5-fluoro-2-pyridyl)-5,5-dimethyl-4,6-dihydropyrrolo[1,2-b]pyrazol-3-yl]-6-methyl-1H-pyrrolo[2,3-b]pyridine (Example 235), except using 3-bromo-6-fluoro-2-(5-fluoropyridin-2-yl)-6-(methoxymethyl)-4,5,6,7-tetrahydropyrazolo[1,5-a]pyridine (Intermediate 61) instead of Intermediate 152. Purification (via chiral SFC, Phenomenex-Cellulose-2 (250 mm×30 mm, 10 um) (isocratic elution: MeOH (containing 0.1% aq. NH₃): supercritical CO₂, 35%: 65% to 35%: 65% (v/v)) afforded the title compound: MS (ESI): mass calcd. for C₂₂H₂₁F₂N₅O 409.2; m/z found 410.1 [M+H]⁺; ¹H NMR (400 MHz, CDCl₃): δ 10.26 (s, 1H), 8.43 (d, J=2.8 Hz, 1H), 7.23-7.12 (m, 3H), 6.89 (s, 1H), 5.98 (s, 1H), 4.71-4.60 (m, 1H), 4.46-4.31 (m, 1H), 3.73-3.62 (m, 2H), 3.49 (s, 3H), 3.09-2.93 (m, 1H), 2.86-2.76 (m, 1H), 2.67 (s, 3H), 2.32-2.20 (m, 1H), 2.06-1.84 (m, 1H); and (*R)-4-[6-Fluoro-2-(5-fluoro-2-pyridyl)-6-(methoxymethyl)-5,7-dihydro-4H-pyrazolo[1,5-a]pyridin-3-yl]-6-methyl-1H-pyrrolo[2,3-b]pyridine (Example 299).

### Example 299: (*R)-4-[6-Fluoro-2-(5-fluoro-2-pyridyl)-6-(methoxymethyl)-5,7-dihydro-4H-pyrazolo[1,5-a]pyridin-3-yl]-6-methyl-1H-pyrrolo[2,3-b]pyridine

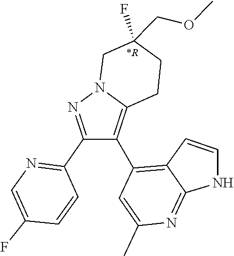 [SMILES: COC[C@@]1(F)CCc2c(-c3cc(C)nc4[nH]ccc34)c(-c3ccc(F)cn3)nn2C1]

The title compound was isolated from chiral SFC purification of Example 298. MS (ESI): mass calcd. for C₂₂H₂₁F₂N₅O 409.2; m/z found 410.1 [M+H]⁺. ¹H NMR (400 MHz, CDCl₃): δ 9.71 (s, 1H), 8.43 (d, J=2.8 Hz, 1H), 7.23-7.09 (m, 3H), 6.89 (s, 1H), 5.98 (s, 1H), 4.70-4.59 (m, 1H), 4.46-4.32 (m, 1H), 3.73-3.62 (m, 2H), 3.49 (s, 3H), 3.08-2.95 (m, 1H), 2.86-2.76 (m, 1H), 2.66 (s, 3H), 2.31-2.17 (m, 1H), 2.06-1.84 (m, 1H).

### Example 300: (*S)-4-[6-Fluoro-2-(5-fluoro-2-pyridyl)-6-(methoxymethyl)-5,7-dihydro-4H-pyrazolo[1,5-a]pyridin-3-yl]-1H-pyrazolo[3,4-b]pyridine

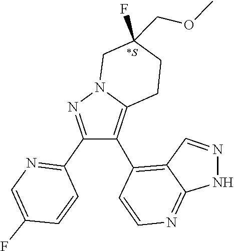 [SMILES: COC[C@]1(F)CCc2c(-c3ccnc4[nH]ncc34)c(-c3ccc(F)cn3)nn2C1]

The title compound was prepared in a manner analogous to Example 1, Steps A-B, except using 3-bromo-6-fluoro-2-(5-fluoropyridin-2-yl)-6-(methoxymethyl)-4,5,6,7-tetrahydropyrazolo[1,5-a]pyridine (Intermediate 61) instead of 3-bromo-2-(5-fluoropyridin-2-yl)-6,7-dihydro-4H-pyrazolo[5,1-c][1,4]oxazine (Intermediate 37) and 4-(4,4,5,5-tetramethyl-1,3,2-dioxaborolan-2-yl)-1-((2-(trimethylsilyl)ethoxy)methyl)-1H-pyrazolo[3,4-b]pyridine (Intermediate 23) instead of 4-(4,4,5,5-tetramethyl-1,3,2-dioxaborolan-2-yl)-1-((2-(trimethylsilyl)ethoxy)methyl)-1H-pyrrolo[2,3-b]pyridine (Intermediate 21) in Step A. Purification (SFC, DAICEL CHIRALCEL OD-H (250 mm×30 mm, 5 um) (isocratic elution: EtOH (containing 0.1% aq. NH₃): supercritical CO₂, 30%: 70% to 30%: 70% (v/v)) afforded the title compound: MS (ESI): mass calcd. for C₂₀H₁₈F₂N₆O 396.2; m/z found 397.1 [M+H]⁺. ¹H NMR (400 MHz, DMSO-d₆): δ 13.50 (s, 1H), 8.46 (d, J=4.5 Hz, 1H), 8.22 (d, J=2.5 Hz, 1H), 7.86-7.80 (m, 1H), 7.79-7.72 (m, 1H), 7.29 (s, 1H), 7.10 (d, J=4.8 Hz, 1H), 4.49-4.47 (m, 1H), 4.46-4.34 (m, 1H), 3.73 (s, 1H), 3.68 (s, 1H), 3.40 (s, 3H), 2. 98-2.78 (m, 2H), 2.26-2.15 (m, 1H), 2.10-1.88 (m, 1H); and (*R)-4-[6-Fluoro-2-(5-fluoro-2-pyridyl)-6-(methoxymethyl)-5,7-dihydro-4H-pyrazolo[1,5-a]pyridin-3-yl]-1H-pyrazolo[3,4-b]pyridine (Example 301).

### Example 301: (*R)-4-[6-Fluoro-2-(5-fluoro-2-pyridyl)-6-(methoxymethyl)-5,7-dihydro-4H-pyrazolo[1,5-a]pyridin-3-yl]-1H-pyrazolo[3,4-b]pyridine

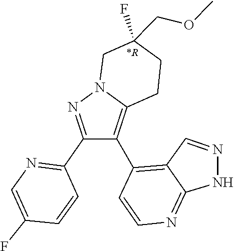 [SMILES: COC[C@@]1(F)CCc2c(-c3ccnc4[nH]ncc34)c(-c3ccc(F)cn3)nn2C1]

The title compound was isolated from chiral SFC purification of Example 300. MS (ESI): mass calcd. for C₂₀H₁₈F₂N₆O 396.2; m/z found 397.1 [M+H]⁺. ¹H NMR (400 MHz, DMSO-d₆): δ 13.50 (s, 1H), 8.46 (d, J=4.8 Hz, 1H), 8.22 (d, J=2.8 Hz, 1H), 7.86-7.80 (m, 1H), 7.79-7.72 (m, 1H), 7.29 (s, 1H), 7.10 (d, J=4.8 Hz, 1H), 4.51-4.47 (m, 1H), 4.46-4.33 (m, 1H), 3.73 (s, 1H), 3.68 (s, 1H), 3.40 (s, 3H), 2.96-2.80 (m, 2H), 2.26-2.16 (m, 1H), 2.10-1.90 (m, 1H).

### Example 302: (*S)-5-Fluoro-4-[6-fluoro-2-(5-fluoro-2-pyridyl)-6-(methoxymethyl)-5,7-dihydro-4H-pyrazolo[1,5-a]pyridin-3-yl]-1H-pyrazolo[3,4-b]pyridine

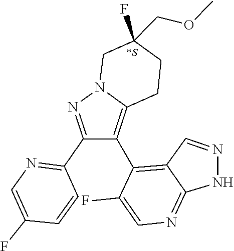 [SMILES: COC[C@]1(F)CCc2c(-c3c(F)cnc4[nH]ncc34)c(-c3ccc(F)cn3)nn2C1]

The title compound was prepared in a manner analogous to Example 1, Steps A-B, except using 3-Bromo-6-fluoro-2-(5-fluoropyridin-2-yl)-6-(methoxymethyl)-4,5,6,7-tetrahydropyrazolo[1,5-a]pyridine (Intermediate 61) instead of 3-bromo-2-(5-fluoropyridin-2-yl)-6,7-dihydro-4H-pyrazolo[5,1-c][1,4]oxazine (Intermediate 37), 5-fluoro-4-(4,4,5,5-tetramethyl-1,3,2-dioxaborolan-2-yl)-1-((2-(trimethylsilyl)ethoxy)methyl)-1H-pyrazolo[3,4-b]pyridine (Intermediate 222) instead of 4-(4,4,5,5-tetramethyl-1,3,2-dioxaborolan-2-yl)-1-((2-(trimethylsilyl)ethoxy)methyl)-1H-pyrrolo[2,3-b]pyridine (Intermediate 21), and Pd(tBu₃P)₂ instead of CataCXium® A Pd G3 in Step A. Purification (chiral SFC; DAICEL CHIRALCEL OD-H 250 mm×30 mm, 5 μm (isocratic elution: EtOH (containing 0.1% of 25% aq. NH₃): supercritical CO₂, 25%: 75% to 25%: 75% (v/v)) afforded the title compound: MS (ESI): mass calcd. for C₂₀H₁₇F₃N₆O 414.1; m/z found 415.3 [M+H]⁺; ¹H NMR (400 MHz, DMSO-d₆): δ 13.75-13.71 (m, 1H), 8.55-8.50 (m, 1H), 8.20-8.18 (m, 1H), 7.94-7.91 (m, 1H), 7.82-7.57 (m, 2H), 4.54-4.34 (m, 2H), 3.76-3.66 (m, 2H), 3.40 (s, 3H), 2.78-2.63 (m, 2H), 2.26-2.16 (m, 1H), 2.12-1.93 (m, 1H); and (*R)-5-Fluoro-4-[6-fluoro-2-(5-fluoro-2-pyridyl)-6-(methoxymethyl)-5,7-dihydro-4H-pyrazolo[1,5-a]pyridin-3-yl]-1H-pyrazolo[3,4-b]pyridine (Example 303).

### Example 303: (*R)-5-Fluoro-4-[6-fluoro-2-(5-fluoro-2-pyridyl)-6-(methoxymethyl)-5,7-dihydro-4H-pyrazolo[1,5-a]pyridin-3-yl]-1H-pyrazolo[3,4-b]pyridine

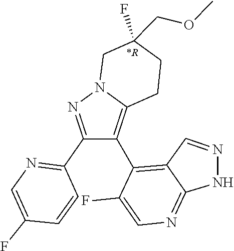 [SMILES: COC[C@@]1(F)CCc2c(-c3c(F)cnc4[nH]ncc34)c(-c3ccc(F)cn3)nn2C1]

The title compound was isolated from chiral SFC purification of Example 302. MS (ESI): mass calcd. for C₂₀H₁₇F₃N₆O 414.1; m/z found, 415.3 [M+H]⁺. ¹H NMR (400 MHz, DMSO-d₆): δ 13.75-13.71 (m, 1H), 8.55-8.50 (m, 1H), 8.20-8.18 (m, 1H), 8.01-7.84 (m, 1H), 7.82-7.57 (m, 2H), 4.58-4.36 (m, 2H), 3.79-3.64 (m, 2H), 3.40 (s, 3H), 2.94-2.59 (m, 2H), 2.25-2.16 (m, 1H), 2.12-1.93 (m, 1H).

### Example 304: (*S)-4-[6-Fluoro-2-(5-fluoro-2-pyridyl)-6-(methoxymethyl)-5,7-dihydro-4H-pyrazolo[1,5-a]pyridin-3-yl]-6-methyl-1H-pyrazolo[3,4-b]pyridine

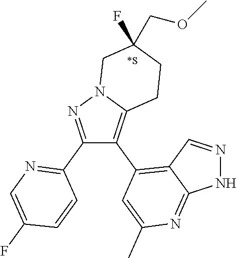 [SMILES: COC[C@]1(F)CCc2c(-c3cc(C)nc4[nH]ncc34)c(-c3ccc(F)cn3)nn2C1]

The title compound was prepared in a manner analogous to Example 1, Steps A-B, except using 3-bromo-6-fluoro-2-(5-fluoropyridin-2-yl)-6-(methoxymethyl)-4,5,6,7-tetrahydropyrazolo[1,5-a]pyridine (Intermediate 61) instead of 3-bromo-2-(5-fluoropyridin-2-yl)-6,7-dihydro-4H-pyrazolo[5,1-c][1,4]oxazine (Intermediate 37), 6-methyl-4-(4,4,5,5-tetramethyl-1,3,2-dioxaborolan-2-yl)-1-((2-(trimethylsilyl)ethoxy)methyl)-1H-pyrazolo[3,4-b]pyridine (Intermediate 26) instead of 4-(4,4,5,5-tetramethyl-1,3,2-dioxaborolan-2-yl)-1-((2-(trimethylsilyl)ethoxy)methyl)-1H-pyrrolo[2,3-b]pyridine (Intermediate 21), and Pd(dppf)₂Cl₂ instead of CataCXium® A Pd G3 in Step A. Purification (SFC: DAICEL CHIRALPAK AD 250 mm×30 mm, 5 μm (isocratic elution: EtOH (containing 0.1% of 25% aq. NH₃): supercritical CO₂, 45%: 55% to 45%: 55% (v/v)) afforded the title compound: MS (ESI): mass calcd. for C₂₁H₂₀F₂N₆O, 410.2; m/z found, 411.1 [M+H]⁺. ¹H NMR (400 MHz, DMSO-d₆): δ 13.26 (s, 1H), 8.23 (d, J=2.8 Hz, 1H), 7.84-7.78 (m, 1H), 7.78-7.70 (m, 1H), 7.13 (s, 1H), 7.02 (s, 1H), 4.55-4.32 (m, 2H), 3.73 (s, 1H), 3.68 (s, 1H), 3.40 (s, 3H), 2.99-2.78 (m, 2H), 2.58 (s, 3H), 2.28-2.13 (m, 1H), 2.11-1.86 (m, 1H); and (*R)-4-[6-Fluoro-2-(5-fluoro-2-pyridyl)-6-(methoxymethyl)-5,7-dihydro-4H-pyrazolo[1,5-a]pyridin-3-yl]-6-methyl-1H-pyrazolo[3,4-b]pyridine (Example 305).

### Example 305: (*R)-4-[6-Fluoro-2-(5-fluoro-2-pyridyl)-6-(methoxymethyl)-5,7-dihydro-4H-pyrazolo[1,5-a]pyridin-3-yl]-6-methyl-1H-pyrazolo[3,4-b]pyridine

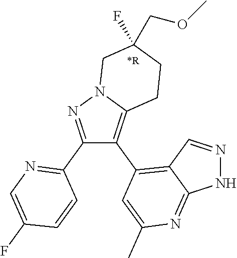 [SMILES: COC[C@@]1(F)CCc2c(-c3cc(C)nc4[nH]ncc34)c(-c3ccc(F)cn3)nn2C1]

The title compound was isolated from chiral SFC purification of Example 304. MS (ESI): mass calcd. for C₂₁H₂₀F₂N₆O 410.2; m/z found 411.1 [M+H]⁺. ¹H NMR (400 MHz, DMSO-d₆): δ 13.26 (s, 1H), 8.23 (d, J=2.8 Hz, 1H), 7.86-7.78 (m, 1H), 7.78-7.69 (m, 1H), 7.13 (s, 1H), 7.02 (s, 1H), 4.58-4.30 (m, 2H), 3.73 (s, 1H), 3.68 (s, 1H), 3.40 (s, 3H), 3.00-2.79 (m, 2H), 2.58 (s, 3H), 2.27-2.15 (m, 1H), 2.10-1.87 (m, 1H).

### Example 306: (*S)-6-(Difluoromethyl)-4-[6-fluoro-2-(5-fluoro-2-pyridyl)-6-(methoxymethyl)-5,7-dihydro-4H-pyrazolo[1,5-a]pyridin-3-yl]-1H-pyrazolo[3,4-b]pyridine

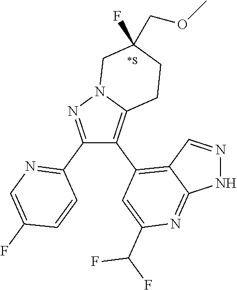 [SMILES: COC[C@]1(F)CCc2c(-c3cc(C(F)F)nc4[nH]ncc34)c(-c3ccc(F)cn3)nn2C1]

The title compound was prepared in a manner analogous to Example 1, Steps A-B, except using 3-bromo-6-fluoro-2-(5-fluoropyridin-2-yl)-6-(methoxymethyl)-4,5,6,7-tetrahydropyrazolo[1,5-a]pyridine (Intermediate 61) instead of 3-bromo-2-(5-fluoropyridin-2-yl)-6,7-dihydro-4H-pyrazolo[5,1-c][1,4]oxazine (Intermediate 37) and 6-(difluoromethyl)-4-(4,4,5,5-tetramethyl-1,3,2-dioxaborolan-2-yl)-1-((2-(trimethylsilyl)ethoxy)methyl)-1H-pyrazolo[3,4-b]pyridine (Intermediate 221) instead of 4-(4,4,5,5-tetramethyl-1,3,2-dioxaborolan-2-yl)-1-((2-(trimethylsilyl)ethoxy)methyl)-1H-pyrrolo[2,3-b]pyridine (Intermediate 21) in Step A. Purification (chiral SFC, DAICEL CHIRALPAK AD 250 mm×30 mm, 10 μm (isocratic elution: EtOH (containing 0.1% of 25% aq. NH₃): supercritical CO₂, 45%: 55% to 45%: 55% (v/v)) afforded the title compound: MS (ESI): mass calcd. for C₂₁H₁₈F₄N₆O 446.1; m/z found 447.1 [M+H]⁺; ¹H NMR (400 MHz, DMSO-d₆): δ 13.82 (s, 1H), 8.19 (d, J=3.0 Hz, 1H), 7.92-7.88 (m, 1H), 7.81-7.76 (m, 1H), 7.50-7.34 (m, 2H), 7.06 (t, J=56.0, 52.0 Hz, 1H), 4.55-4.36 (m, 2H), 3.74 (s, 1H), 3.69 (s, 1H), 3.40 (s, 3H), 3.01-2.78 (m, 2H), 2.28-2.14 (m, 1H), 2.12-1.89 (m, 1H); and (*R)-6-(Difluoromethyl)-4-[6-fluoro-2-(5-fluoro-2-pyridyl)-6-(methoxymethyl)-5,7-dihydro-4H-pyrazolo[1,5-a]pyridin-3-yl]-1H-pyrazolo[3,4-b]pyridine (example 307).

### Example 307: (*R)-6-(Difluoromethyl)-4-[6-fluoro-2-(5-fluoro-2-pyridyl)-6-(methoxymethyl)-5,7-dihydro-4H-pyrazolo[1,5-a]pyridin-3-yl]-1H-pyrazolo[3,4-b]pyridine

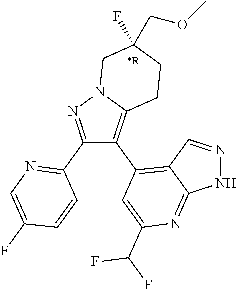 [SMILES: COC[C@@]1(F)CCc2c(-c3cc(C(F)F)nc4[nH]ncc34)c(-c3ccc(F)cn3)nn2C1]

The title compound was isolated from chiral SFC purification of Example 306. MS (ESI): mass calcd. for C₂₁H₁₈F₄N₆O 446.1; m/z found 447.1 [M+H]⁺. ¹H NMR (400 MHz, DMSO-d₆): δ 13.82 (s, 1H), 8.25-8.13 (m, 1H), 7.91-7.88 (m, 1H), 7.81-7.76 (m, 1H), 7.47-7.34 (m, 2H), 7.06 (t, J=56, 52 Hz, 1H), 4.56-4.29 (m, 2H), 3.74 (s, 1H), 3.69 (s, 1H), 3.40 (s, 3H), 3.05-2.82 (m, 2H), 2.29-2.16 (m, 1H), 2.10-1.84 (m, 1H).

### Example 308: (*S)-5-Fluoro-4-[6-fluoro-2-(5-fluoro-2-pyridyl)-6-(methoxymethyl)-5,7-dihydro-4H-pyrazolo[1,5-a]pyridin-3-yl]-6-methyl-1H-pyrazolo[3,4-b]pyridine

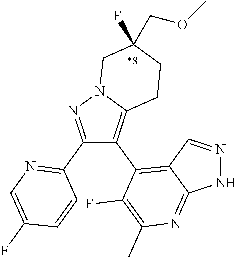 [SMILES: COC[C@]1(F)CCc2c(-c3c(F)c(C)nc4[nH]ncc34)c(-c3ccc(F)cn3)nn2C1]

The title compound was prepared in a manner analogous to Example 1, Steps A-B, except using 3-Bromo-6-fluoro-2-(5-fluoropyridin-2-yl)-6-(methoxymethyl)-4,5,6,7-tetrahydropyrazolo[1,5-a]pyridine (Intermediate 61) instead of 3-bromo-2-(5-fluoropyridin-2-yl)-6,7-dihydro-4H-pyrazolo[5,1-c][1,4]oxazine (Intermediate 37) and 5-fluoro-6-methyl-4-(4,4,5,5-tetramethyl-1,3,2-dioxaborolan-2-yl)-1-((2-(trimethylsilyl)ethoxy)methyl)-1H-pyrazolo[3,4-b]pyridine (Intermediate 223) instead of 4-(4,4,5,5-tetramethyl-1,3,2-dioxaborolan-2-yl)-1-((2-(trimethylsilyl)ethoxy)methyl)-1H-pyrrolo[2,3-b]pyridine (Intermediate 21) in Step A. Purification (chiral SFC: DAICEL CHIRALPAK AD 250 mm×30 mm, 10 μm (isocratic elution: EtOH (containing 0.1% of 25% aq. NH₃): supercritical CO₂, 35%: 65% to 35%: 65% (v/v))) afforded the title compound: MS (ESI): mass calcd. for C₂₁H₁₉F₃N₆O 428.2; m/z found 429.1 [M+H]⁺; ¹H NMR (400 MHz, DMSO-d₆): δ 13.50 (d, J=14.3 Hz, 1H), 8.20-8.18 (m, 1H), 7.99-7.85 (m, 1H), 7.78-7.72 (m, 1H), 7.62-7.37 (m, 1H), 4.59-4.36 (m, 2H), 3.76-3.67 (m, 2H), 3.40 (s, 3H), 2.86-2.63 (m, 2H), 2.62-2.52 (m, 3H), 2.28-2.15 (m, 1H), 2.14-1.91 (m, 1H); and (*R)-5-Fluoro-4-[6-fluoro-2-(5-fluoro-2-pyridyl)-6-(methoxymethyl)-5,7-dihydro-4H-pyrazolo[1,5-a]pyridin-3-yl]-6-methyl-1H-pyrazolo[3,4-b]pyridine (Example 309).

### Example 309: (*R)-5-Fluoro-4-[6-fluoro-2-(5-fluoro-2-pyridyl)-6-(methoxymethyl)-5,7-dihydro-4H-pyrazolo[1,5-a]pyridin-3-yl]-6-methyl-1H-pyrazolo[3,4-b]pyridine

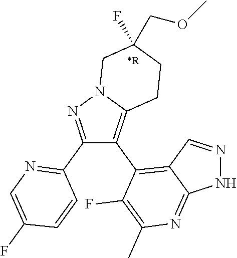 [SMILES: COC[C@@]1(F)CCc2c(-c3c(F)c(C)nc4[nH]ncc34)c(-c3ccc(F)cn3)nn2C1]

The title compound was isolated from chiral SFC purification of Example 308. MS (ESI): mass calcd. for C₂₁H₁₉F₃N₆O 428.2; m/z found 429.1 [M+H]⁺. ¹H NMR (400 MHz, DMSO-d₆): δ 13.55-13.44 (m, 1H), 8.33-8.14 (m, 1H), 7.97-7.87 (m, 1H), 7.80-7.70 (m, 1H), 7.61-7.36 (m, 1H), 4.57-4.32 (m, 2H), 3.84-3.62 (m, 2H), 3.40 (s, 3H), 2.82-2.63 (m, 2H), 2.57-2.52 (m, 3H), 2.25-2.14 (m, 1H), 2.12-1.86 (m, 1H).

### Example 310: (*S)-4-(6-Fluoro-2-(5-fluoropyridin-2-yl)-6-((methoxy-d₃)methyl)-4,5,6,7-tetrahydropyrazolo[1,5-a]pyridin-3-yl)-6-methyl-1H-pyrazolo[3,4-b]pyridine

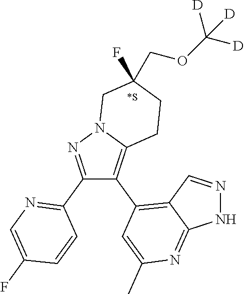 [SMILES: [2H]C([2H])([2H])OC[C@]1(F)CCc2c(-c3cc(C)nc4[nH]ncc34)c(-c3ccc(F)cn3)nn2C1]

Title compound was prepared in a manner analogous to Example 1, Steps A-B, except using 3-bromo-6-fluoro-2-(5-fluoropyridin-2-yl)-6-((methoxy-d₃)methyl)-4,5,6,7-tetrahydropyrazolo[1,5-a]pyridine (Intermediate 171) instead of 3-bromo-2-(5-fluoropyridin-2-yl)-6,7-dihydro-4H-pyrazolo[5,1-c][1,4]oxazine (Intermediate 37) and 6-methyl-4-(4,4,5,5-tetramethyl-1,3,2-dioxaborolan-2-yl)-1-((2-(trimethylsilyl)ethoxy)methyl)-1H-pyrazolo[3,4-b]pyridine (Intermediate 26) instead of 4-(4,4,5,5-tetramethyl-1,3,2-dioxaborolan-2-yl)-1-((2-(trimethylsilyl)ethoxy)methyl)-1H-pyrrolo[2,3-b]pyridine (Intermediate 21) in Step A. Purification (chiral SFC: DAICEL CHIRALPAK AD 250 mm×30 mm, 10 μm (isocratic elution: EtOH (containing 0.1% of 25% aq. NH₃): supercritical CO₂, 55%: 45% to 55%: 45% (v/v))) afforded the title compound: MS (ESI): mass calcd. for C₂₁H₁₇D₃F₂N₆O 413.2; m/z found 414.1 [M+H]⁺. ¹H NMR (400 MHz, DMSO-d₆): δ 13.25 (s, 1H), 8.22 (d, J=2.9 Hz, 1H), 7.86-7.78 (m, 1H), 7.77-7.68 (m, 1H), 7.13 (d, J=1.2 Hz, 1H), 7.02 (s, 1H), 4.50-4.32 (m, 2H), 3.73 (d, J=1.7 Hz, 1H), 3.68 (s, 1H), 2.98-2.80 (m, 2H), 2.58 (s, 3H), 2.27-2.15 (m, 1H), 2.09-1.86 (m, 1H); and (*R)-4-(6-Fluoro-2-(5-fluoropyridin-2-yl)-6-((methoxy-d₃)methyl)-4,5,6,7-tetrahydropyrazolo[1,5-a]pyridin-3-yl)-6-methyl-1H-pyrazolo[3,4-b]pyridine (Example 311).

### Example 311: (*R)-4-(6-Fluoro-2-(5-fluoropyridin-2-yl)-6-((methoxy-d₃)methyl)-4,5,6,7-tetrahydropyrazolo[1,5-a]pyridin-3-yl)-6-methyl-1H-pyrazolo[3,4-b]pyridine

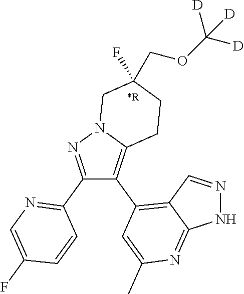 [SMILES: [2H]C([2H])([2H])OC[C@@]1(F)CCc2c(-c3cc(C)nc4[nH]ncc34)c(-c3ccc(F)cn3)nn2C1]

The title compound was isolated from chiral SFC purification of Example 310. MS (ESI): mass calcd. for C₂₁H₁₇D₃F₂N₆O 413.2; m/z found 414.1 [M+H]⁺. ¹H NMR (400 MHz, DMSO-d₆): δ 13.26 (s, 1H), 8.23 (d, J=2.9 Hz, 1H), 7.85-7.78 (m, 1H), 7.77-7.69 (m, 1H), 7.13 (s, 1H), 7.02 (s, 1H), 4.51-4.34 (m, 2H), 3.73 (d, J=1.4 Hz, 1H), 3.67 (s, 1H), 2.97-2.81 (m, 2H), 2.58 (s, 3H), 2.28-2.14 (m, 1H), 2.10-1.83 (m, 1H).

### Example 312: (*S)-4-[6-[(Methoxy-d₃)methyl-d₂]-6-fluoro-2-(5-fluoro-2-pyridyl)-5,7-dihydro-4H-pyrazolo[1,5-a]pyridin-3-yl]-6-methyl-1H-pyrazolo[3,4-b]pyridine

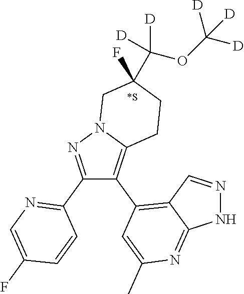 [SMILES: [2H]C([2H])([2H])OC([2H])([2H])[C@]1(F)CCc2c(-c3cc(C)nc4[nH]ncc34)c(-c3ccc(F)cn3)nn2C1]

The title compound was prepared in a manner analogous to Example 1, Steps A-B, except using 3-bromo-6-fluoro-2-(5-fluoropyridin-2-yl)-6-((methoxy-d₃)methyl-d₂)-4,5,6,7-tetrahydropyrazolo[1,5-a]pyridine (Intermediate 172) instead of 3-bromo-2-(5-fluoropyridin-2-yl)-6,7-dihydro-4H-pyrazolo[5,1-c][1,4]oxazine (Intermediate 37) and 6-methyl-4-(4,4,5,5-tetramethyl-1,3,2-dioxaborolan-2-yl)-1-((2-(trimethylsilyl)ethoxy)methyl)-1H-pyrazolo[3,4-b]pyridine (Intermediate 26) instead of 4-(4,4,5,5-tetramethyl-1,3,2-dioxaborolan-2-yl)-1-((2-(trimethylsilyl)ethoxy)methyl)-1H-pyrrolo[2,3-b]pyridine (Intermediate 21) in Step A. Purification (chiral SFC: DAICEL CHIRALPAK AD 250 mm×30 mm, 10 μm (isocratic elution: EtOH (containing 0.1% of 25% aq. NH₃): supercritical CO₂, 55%: 45% to 55%: 45% (v/v))) afforded the title compound: MS (ESI): mass calcd. for C₂₁H₁₅D₅F₂N₆O 415.2; m/z found 416.1 [M+H]⁺; ¹H NMR (400 MHz, DMSO-d₆): δ 13.26 (s, 1H), 8.23-8.21 (m, 1H), 7.85-7.71 (m, 2H), 7.13 (s, 1H), 7.02 (s, 1H), 4.52-4.34 (m, 2H), 3.02-2.80 (m, 2H), 2.58 (s, 3H), 2.28-2.16 (m, 1H), 2.07-1.88 (m, 1H); and (*R)-4-[6-[(methoxy-d₃)methyl-d₂]-6-fluoro-2-(5-fluoro-2-pyridyl)-5,7-dihydro-4H-pyrazolo[1,5-a]pyridin-3-yl]-6-methyl-1H-pyrazolo[3,4-b]pyridine (Example 313).

### Example 313: (*R)-4-[6-[(Methoxy-d₃)methyl-d₂]-6-fluoro-2-(5-fluoro-2-pyridyl)-5,7-dihydro-4H-pyrazolo[1,5-a]pyridin-3-yl]-6-methyl-1H-pyrazolo[3,4-b]pyridine

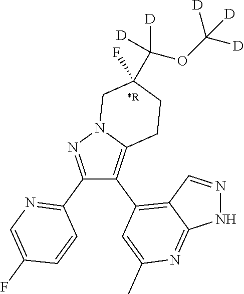 [SMILES: [2H]C([2H])([2H])OC([2H])([2H])[C@@]1(F)CCc2c(-c3cc(C)nc4[nH]ncc34)c(-c3ccc(F)cn3)nn2C1]

The title compound was isolated from chiral SFC purification of Example 312. MS (ESI): mass calcd. for C₂₁H₁₅D₅F₂N₆O 415.2; m/z found 416.1 [M+H]⁺. ¹H NMR (400 MHz, DMSO-d₆): δ 13.26 (s, 1H), 8.23-8.21 (m, 1H), 7.95-7.59 (m, 2H), 7.13 (s, 1H), 7.01 (s, 1H), 4.59-4.32 (m, 2H), 3.02-2.78 (m, 2H), 2.57 (s, 3H), 2.22-2.20 (m, 1H), 2.08-1.84 (m, 1H).

### Example 314: (*S)-4-(6-((Difluoromethoxy)methyl)-6-fluoro-2-(5-fluoropyridin-2-yl)-4,5,6,7-tetrahydropyrazolo[1,5-a]pyridin-3-yl)-1H-pyrazolo[3,4-b]pyridine

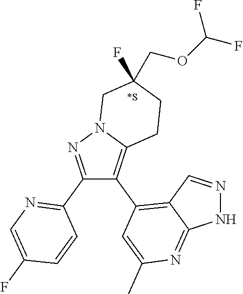 [SMILES: Cc1cc(-c2c(-c3ccc(F)cn3)nn3c2CC[C@@](F)(COC(F)F)C3)c2cn[nH]c2n1]

The title compound was prepared in a manner analogous to Example 1, Steps A-B, except using 3-bromo-6-((difluoromethoxy)methyl)-6-fluoro-2-(5-fluoropyridin-2-yl)-4,5,6,7-tetrahydropyrazolo[1,5-a]pyridine (Intermediate 173) instead of 3-bromo-2-(5-fluoropyridin-2-yl)-6,7-dihydro-4H-pyrazolo[5,1-c][1,4]oxazine (Intermediate 37) and 4-(4,4,5,5-Tetramethyl-1,3,2-dioxaborolan-2-yl)-1-((2-(trimethylsilyl)ethoxy)methyl)-1H-pyrazolo[3,4-b]pyridine (Intermediate 23) instead of 4-(4,4,5,5-tetramethyl-1,3,2-dioxaborolan-2-yl)-1-((2-(trimethylsilyl)ethoxy)methyl)-1H-pyrrolo[2,3-b]pyridine (Intermediate 21) in Step A. Purification (chiral SFC: DAICEL CHIRALPAK AD 250 mm×30 mm, 10 μm (isocratic elution: EtOH (containing 0.1% of 25% aq. NH₃): supercritical CO₂, 50%: 50% to 50%: 50% (v/v)) afforded the title compound: MS (ESI): mass calcd. for C₂₀H₁₆F₄N₆O 432.1; m/z found, 433.0 [M+H]⁺; ¹H NMR (400 MHz, DMSO-d₆): δ 13.52 (s, 1H), 8.47 (d, J=4.8 Hz, 1H), 8.23 (d, J=2.5 Hz, 1H), 8.00-7.60 (m, 2H), 7.29 (s, 1H), 7.11 (d, J=4.8 Hz, 1H), 6.84 (t, J=76.0 Hz, 76.0 Hz, 1H), 4.64-4.38 (m, 2H), 4.36-4.16 (m, 2H), 3.02-2.79 (m, 2H), 2. 35-2.24 (m, 1H), 2.14-1.90 (m, 1H); and (*R)-4-(6-((Difluoromethoxy)methyl)-6-fluoro-2-(5-fluoropyridin-2-yl)-4,5,6,7-tetrahydropyrazolo[1,5-a]pyridin-3-yl)-1H-pyrazolo[3,4-b]pyridine (Example 315).

### Example 315: (*R)-4-(6-((Difluoromethoxy)methyl)-6-fluoro-2-(5-fluoropyridin-2-yl)-4,5,6,7-tetrahydropyrazolo[1,5-a]pyridin-3-yl)-1H-pyrazolo[3,4-b]pyridine

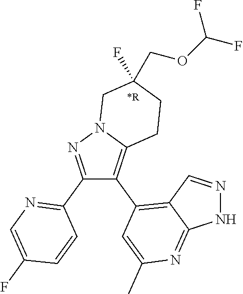 [SMILES: Cc1cc(-c2c(-c3ccc(F)cn3)nn3c2CC[C@](F)(COC(F)F)C3)c2cn[nH]c2n1]

The title compound was isolated from chiral SFC purification of Example 314. MS (ESI): mass calcd. for C₂₀H₁₆F₄N₆O 432.1; m/z found 433.1 [M+H]⁺. ¹H NMR (400 MHz, DMSO-d₆): δ 13.52 (s, 1H), 8.47 (d, J=4.5 Hz, 1H), 8.23 (s, 1H), 7.98-7.65 (m, 2H), 7.29 (s, 1H), 7.11 (d, J=4.5 Hz, 1H), 6.84 (t, J=76.0 Hz, 76.0 Hz, 1H), 4.62-4.38 (m, 2H), 4.35-4.19 (m, 2H), 3.06-2.78 (m, 2H), 2.36-2.23 (m, 1H), 2.18-1.88 (m, 1H).

### Example 316: (*S)-4-[6-Fluoro-2-(5-fluoro-2-pyridyl)-6-(rifluoromethoxymethyl)-5,7-dihydro-4H-pyrazolo[1,5-a]pyridin-3-yl]-1H-pyrazolo[3,4-b]pyridine

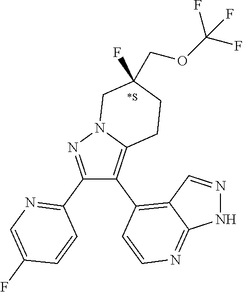 [SMILES: Fc1ccc(-c2nn3c(c2-c2ccnc4[nH]ncc24)CC[C@@](F)(COC(F)(F)F)C3)nc1]

The title compound was prepared in a manner analogous to Example 1, Steps A-B, except using: 3-bromo-6-fluoro-2-(5-fluoropyridin-2-yl)-6-((trifluoromethoxy)methyl)-4,5,6,7-tetrahydropyrazolo[1,5-a]pyridine (Intermediate 174) instead of 3-bromo-2-(5-fluoropyridin-2-yl)-6,7-dihydro-4H-pyrazolo[5,1-c][1,4]oxazine (Intermediate 37) and 4-(4,4,5,5-tetramethyl-1,3,2-dioxaborolan-2-yl)-1-((2-(trimethylsilyl)ethoxy)methyl)-1H-pyrazolo[3,4-b]pyridine (Intermediate 23) instead of 4-(4,4,5,5-tetramethyl-1,3,2-dioxaborolan-2-yl)-1-((2-(trimethylsilyl)ethoxy)methyl)-1H-pyrrolo[2,3-b]pyridine (Intermediate 21) in Step A. Purification (chiral SFC: DAICEL CHIRALPAK AD 250 mm×30 mm, 10 μm (isocratic elution: EtOH (containing 0.1% of 25% aq. NH₃): supercritical CO₂, 45%: 55% to 45%: 55% (v/v)) afforded the title compound: MS (ESI): mass calcd. for C₂₀H₁₅F₅N₆O 450.1; m/z found 451.0 [M+H]⁺; ¹H NMR (400 MHz, DMSO-d₆) δ 13.52 (s, 1H), 8.47 (s, 1H), 8.25-8.22 (m, 1H), 7.95-7.63 (m, 2H), 7.31-7.29 (m, 1H), 7.11 (s, 1H), 4.69-4.34 (m, 4H), 3.01-2.80 (m, 2H), 2.40-2.28 (m, 1H), 2.19-1.91 (m, 1H); and (*R)-4-[6-Fluoro-2-(5-fluoro-2-pyridyl)-6-(trifluoromethoxymethyl)-5,7-dihydro-4H-pyrazolo[1,5-a]pyridin-3-yl]-1H-pyrazolo[3,4-b]pyridine (Example 317).

### Example 317: (*R)-4-[6-Fluoro-2-(5-fluoro-2-pyridyl)-6-(trifluoromethoxymethyl)-5,7-dihydro-4H-pyrazolo[1,5-a]pyridin-3-yl]-1H-pyrazolo[3,4-b]pyridine

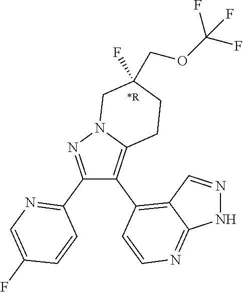 [SMILES: Fc1ccc(-c2nn3c(c2-c2ccnc4[nH]ncc24)CC[C@](F)(COC(F)(F)F)C3)nc1]

The title compound was isolated from chiral SFC purification of Example 316. MS (ESI): mass calcd. for C₂₀H₁₅F₅N₆O 450.1; m/z found, 451.0 [M+H]⁺. ¹H NMR (400 MHz, DMSO-d₆) δ 13.52 (s, 1H), 8.47 (s, 1H), 8.25-8.22 (m, 1H), 7.96-7.63 (m, 2H), 7.32-7.29 (m, 1H), 7.12 (s, 1H), 4.62-4.40 (m, 4H), 3.01-2.81 (m, 2H), 2.40-2.29 (m, 1H), 2.19-1.89 (m, 1H).

### Example 318: (*S)-4-[2-(5-Fluoro-2-pyridyl)-6-(2-methoxyethyl)-6-methyl-5,7-dihydro-4H-pyrazolo[1,5-a]pyridin-3-yl]-1H-pyrazolo[3,4-b]pyridine

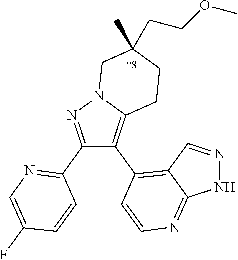 [SMILES: COCC[C@]1(C)CCc2c(-c3ccnc4[nH]ncc34)c(-c3ccc(F)cn3)nn2C1]

The title compound was prepared in a manner analogous to Example 1, Steps A-B, except using 3-bromo-2-(5-fluoropyridin-2-yl)-6-(2-methoxyethyl)-6-methyl-4,5,6,7-tetrahydropyrazolo[1,5-a]pyridine (Intermediate 166) instead of 3-bromo-2-(5-fluoropyridin-2-yl)-6,7-dihydro-4H-pyrazolo[5,1-c][1,4]oxazine (Intermediate 37); and 4-(4,4,5,5-tetramethyl-1,3,2-dioxaborolan-2-yl)-1-((2-(trimethylsilyl)ethoxy)methyl)-1H-pyrazolo[3,4-b]pyridine (Intermediate 23) instead of 4-(4,4,5,5-tetramethyl-1,3,2-dioxaborolan-2-yl)-1-((2-(trimethylsilyl)ethoxy)methyl)-1H-pyrrolo[2,3-b]pyridine (Intermediate 21) in Step A. Purification (chiral SFC (DAICEL CHIRALPAK AD 250 mm×30 mm, 10 μm (isocratic elution: MeOH (containing 0.1% of 25% aq. NH₃): supercritical CO₂, 60%: 40% to 60%: 40% (v/v)) afforded the title compound: MS (ESI): mass calcd. For C₂₂H₂₃FN₆O 406.2; m/z found 407.1 [M+H]⁺; ¹H NMR (400 MHz, DMSO-d₆): δ 13.47 (s, 1H), 8.45 (d, J=4.8 Hz, 1H), 8.20 (d, J=2.8 Hz, 1H), 7.89-7.79 (m, 1H), 7.78-7.70 (m, 1H), 7.30 (s, 1H), 7.09 (d, J=4.5 Hz, 1H), 4.12-4.03 (m, 1H), 4.00-3.91 (m, 1H), 3.51-3.48 (m, 2H), 3.25 (s, 3H), 2.78 (t, J=6.4 Hz, 2H), 1.77-1.60 (m, 4H), 1.09 (s, 3H); and (*R)-4-[2-(5-Fluoro-2-pyridyl)-6-(2-methoxyethyl)-6-methyl-5,7-dihydro-4H-pyrazolo[1,5-a]pyridin-3-yl]-1H-pyrazolo[3,4-b]pyridine (Example 319).

### Example 319: (*R)-4-[2-(5-Fluoro-2-pyridyl)-6-(2-methoxyethyl)-6-methyl-5,7-dihydro-4H-pyrazolo[1,5-a]pyridin-3-yl]-1H-pyrazolo[3,4-b]pyridine

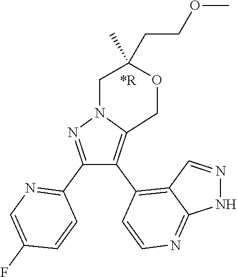 [SMILES: COCC[C@]1(C)Cn2nc(-c3ccc(F)cn3)c(-c3ccnc4[nH]ncc34)c2CO1]

The title compound was isolated from chiral SFC purification of Example 318. MS (ESI): mass calcd. For C₂₂H₂₃FN₆O 406.2; m/z found 407.1 [M+H]⁺. ¹H NMR (400 MHz, DMSO-d₆) δ 13.47 (s, 1H), 8.45 (d, J=4.8 Hz, 1H), 8.20 (d, J=2.8 Hz, 1H), 7.88-7.80 (m, 1H), 7.79-7.68 (m, 1H), 7.30 (s, 1H), 7.10 (d, J=4.8 Hz, 1H), 4.13-4.01 (m, 1H), 4.00-3.88 (m, 1H), 3.59-3.45 (m, 2H), 3.25 (s, 3H), 2.78 (t, J=6.5 Hz, 2H), 1.77-1.58 (m, 4H), 1.09 (s, 3H).

### Example 320: (*R)-4-(2-(5-Fluoropyridin-2-yl)-6-methyl-6-(oxetan-3-ylmethyl)-4,5,6,7-tetrahydropyrazolo[1,5-a]pyridin-3-yl)-1H-pyrazolo[3,4-b]pyridine

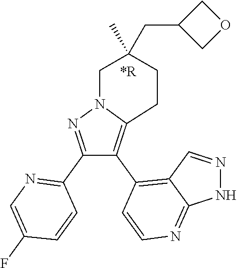 [SMILES: C[C@]1(CC2COC2)CCc2c(-c3ccnc4[nH]ncc34)c(-c3ccc(F)cn3)nn2C1]

The title compound was prepared in a manner analogous to Example 1, Steps A-B, except using racemic 3-bromo-2-(5-fluoropyridin-2-yl)-6-methyl-6-(oxetan-3-ylmethyl)-4,5,6,7-tetrahydropyrazolo[1,5-a]pyridine (Intermediate 167) instead of 3-bromo-2-(5-fluoropyridin-2-yl)-6,7-dihydro-4H-pyrazolo[5,1-c][1,4]oxazine (Intermediate 37); and 4-(4,4,5,5-tetramethyl-1,3,2-dioxaborolan-2-yl)-1-((2-(trimethylsilyl)ethoxy)methyl)-1H-pyrazolo[3,4-b]pyridine (Intermediate 23) instead of 4-(4,4,5,5-tetramethyl-1,3,2-dioxaborolan-2-yl)-1-((2-(trimethylsilyl)ethoxy)methyl)-1H-pyrrolo[2,3-b]pyridine (Intermediate 21) in Step A. Purification (chiral SFC (Phenomenex-Cellulose-2 250 mm×30 mm, 5 μm (isocratic elution: MeOH (containing 0.1% of 25% aq. NH₃): supercritical CO₂, 45%: 55% to 45%: 55% (v/v)) afforded the title compound: MS (ESI): mass calcd. for C₂₃H₂₃FN₆O 418.2; m/z found 419.1 [M+H]⁺; ¹H NMR (400 MHz, CDCl₃): δ 8.56 (s, 1H), 8.26 (s, 1H), 7.73-7.46 (m, 2H), 7.39-7.32 (m, 1H), 7.16 (d, J=4.6 Hz, 1H), 4.59-4.27 (m, 2H), 4.00 (d, J=6.6 Hz, 1H), 3.89-3.83 (m, 1H), 3.30-3.22 (m, 1H), 3.06 (d, J=7.6 Hz, 1H), 2.96 (t, J=11.2 Hz, 1H), 2.84-2.78 (m, 2H), 2.02 (s, 1H), 1.69-1.67 (m, 1H), 0.89-0.84 (m, 6H); and (*S)-4-(2-(5-Fluoropyridin-2-yl)-6-methyl-6-(oxetan-3-ylmethyl)-4,5,6,7-tetrahydropyrazolo[1,5-a]pyridin-3-yl)-1H-pyrazolo[3,4-b]pyridine (Example 321).

### Example 321: (*S)-4-(2-(5-Fluoropyridin-2-yl)-6-methyl-6-(oxetan-3-ylmethyl)-4,5,6,7-tetrahydropyrazolo[1,5-a]pyridin-3-yl)-1H-pyrazolo[3,4-b]pyridine

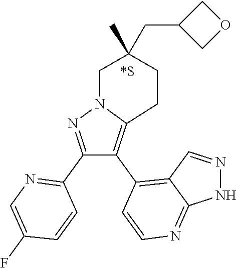 [SMILES: C[C@@]1(CC2COC2)CCc2c(-c3ccnc4[nH]ncc34)c(-c3ccc(F)cn3)nn2C1]

The title compound was isolated from chiral SFC purification of Example 320. MS (ESI): mass calcd. for C₂₃H₂₃FN₆O 418.2; m/z found 419.1 [M+H]⁺. ¹H NMR (400 MHz, CDCl₃): δ 8.64-8.46 (m, 1H), 8.36-8.22 (m, 1H), 7.55-7.35 (m, 2H), 7.24 (d, J=2.9 Hz, 1H), 7.14-7.03 (m, 1H), 4.49-4.25 (m, 1H), 4.00-3.94 (m, 2H), 3.91-3.85 (m, 1H), 3.34-3.19 (m, 1H), 3.00-2.81 (m, 3H), 2.27-2.15 (m, 1H), 1.97-1.84 (m, 2H), 0.92-0.84 (m, 6H).

### Example 322: (Racemic) 2-(5-Fluoropyridin-2-yl)-6-(methyl-d₃)-3-(1H-pyrazolo[3,4-b]pyridin-4-yl)-4,5,6,7-tetrahydropyrazolo[1,5-a]pyridine-6-carbonitrile

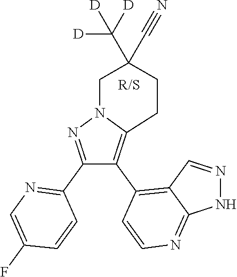 [SMILES: [2H]C([2H])([2H])C1(C#N)CCc2c(-c3ccnc4[nH]ncc34)c(-c3ccc(F)cn3)nn2C1]

The title compound was prepared in a manner analogous to Example 1, Steps A-B, except using 3-bromo-2-(5-fluoropyridin-2-yl)-6-(methyl-d₃)-4,5,6,7-tetrahydropyrazolo[1,5-a]pyridine-6-carbonitrile (Intermediate 170) instead of 3-bromo-2-(5-fluoropyridin-2-yl)-6,7-dihydro-4H-pyrazolo[5,1-c][1,4]oxazine (Intermediate 37) and 4-(4,4,5,5-tetramethyl-1,3,2-dioxaborolan-2-yl)-1-((2-(trimethylsilyl)ethoxy)methyl)-1H-pyrazolo[3,4-b]pyridine (Intermediate 23) instead of 4-(4,4,5,5-tetramethyl-1,3,2-dioxaborolan-2-yl)-1-((2-(trimethylsilyl)ethoxy)methyl)-1H-pyrrolo[2,3-b]pyridine (Intermediate 21) in Step A. Purification was carried out on a basic AQQU prep ACN/20 mM-NH₄OH 0-100% over 25 min. MS (ESI): mass calcd. for C₂₀H₁₃D₃FN₇, 376.1; m/z found, 377.2 [M+H]⁺.

### Example 323: *S-2-(5-Fluoropyridin-2-yl)-6-(methyl-d₃)-3-(1H-pyrazolo[3,4-b]pyridin-4-yl)-4,5,6,7-tetrahydropyrazolo[1,5-a]pyridine-6-carbonitrile

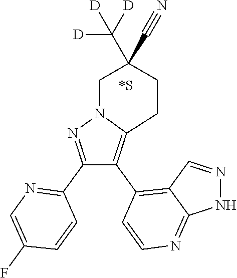 [SMILES: [2H]C([2H])([2H])[C@]1(C#N)CCc2c(-c3ccnc4[nH]ncc34)c(-c3ccc(F)cn3)nn2C1]

The title compound was isolated from chiral SFC purification [(Stationary phase: Chiralpak 1H Sum 250×21 mm, Mobile phase: 25% methanol, 75% CO₂) Flow rate 42 mL/min, monitor at 220 nm] of Example 322. MS (ESI): mass calcd. for C₂₀H₁₃D₃FN₇, 376.1; m/z found, 377.2 [M+H]⁺. ¹H NMR (600 MHz, DMSO-d₆): δ 13.54 (s, 1H), 8.50 (d, J=4.7 Hz, 1H), 8.25 (m, 1H), 7.87 (m, 1H), 7.78 (m, 1H), 7.31 (d, J=1.4 Hz, 1H), 7.14 (d, J=4.7 Hz, 1H), 4.72 (m, 1H), 4.24 (d, J=13.1 Hz, 1H), 2.97 (m, 2H), 2.33-2.25 (m, 1H), 2.02 (m, 1H).

### Example 324: (*R)-2-(S-Fluoropyridin-2-yl)-6-(methyl-d₃)-3-(1H-pyrazolo[3,4-b]pyridin-4-yl)-4,5,6,7-tetrahydropyrazolo[1,5-a]pyridine-6-carbonitrile

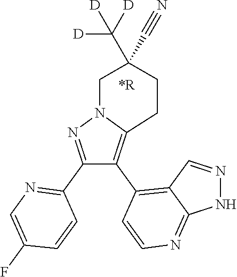 [SMILES: [2H]C([2H])([2H])[C@@]1(C#N)CCc2c(-c3ccnc4[nH]ncc34)c(-c3ccc(F)cn3)nn2C1]

The title compound was isolated from chiral SFC purification [(Stationary phase: Chiralpak 1H Sum 250×21 mm, Mobile phase: 25% methanol, 75% CO₂) Flow rate 42 mL/min, monitor at 220 nm] of Example 322. MS (ESI): mass calcd. for C₂₀H₁₃D₃FN₇, 376.1; m/z found, 377.2 [M+H]⁺. ¹H NMR (600 MHz, DMSO-d₆): δ 13.54 (s, 1H), 8.50 (d, J=4.7 Hz, 1H), 8.25 (m, 1H), 7.87 (m, 1H), 7.78 (m, 1H), 7.31 (d, J=1.4 Hz, 1H), 7.14 (d, J=4.8 Hz, 1H), 4.72 (m, 1H), 4.24 (d, J=13.1 Hz, 1H), 2.97 (m, 2H), 2.32-2.21 (m, 1H), 2.10-1.90 (m, 1H).

### Example 325: (*R)-2,2-Difluoro-2′-(4-fluorophenyl)-3′-(1H-pyrazolo[3,4-b]pyridin-4-yl)-4′,5′-dihydro-7′H-spiro[cyclopropane-1,6′-pyrazolo[1,5-a]pyridine]

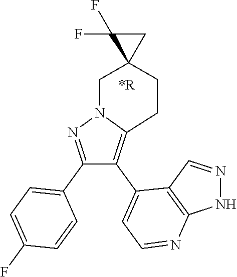 [SMILES: Fc1ccc(-c2nn3c(c2-c2ccnc4[nH]ncc24)CC[C@]2(C3)CC2(F)F)cc1]

The title compound was prepared in a manner analogous to Example 1, Steps A-B, except using 3′-bromo-2,2-difluoro-2′-(4-fluorophenyl)-4′,5′-dihydro-7′H-spiro[cyclopropane-1,6′-pyrazolo[1,5-a]pyridine] (Intermediate 175) instead of 3-bromo-2-(5-fluoropyridin-2-yl)-6,7-dihydro-4H-pyrazolo[5,1-c][1,4]oxazine (Intermediate 37); and 4-(4,4,5,5-tetramethyl-1,3,2-dioxaborolan-2-yl)-1-((2-(trimethylsilyl)ethoxy)methyl)-1H-pyrazolo[3,4-b]pyridine (Intermediate 23) instead of 4-(4,4,5,5-tetramethyl-1,3,2-dioxaborolan-2-yl)-1-((2-(trimethylsilyl)ethoxy)methyl)-1H-pyrrolo[2,3-b]pyridine (Intermediate 21) in Step A. Purification (chiral SFC: [DAICEL CHIRALPAK IG (250 mm×30 mm, 10 m) (isocratic elution: EtOH (containing 0.1% aq. NH₃): supercritical CO₂, 60%: 40% to 60%: 40% (v/v)) afforded the title compound: MS (ESI): mass calcd. for C₂₁H₁₆F₃N₅ 395.1; m/z found 396.2 [M+H]⁺; ¹H NMR (400 MHz, DMSO-d₆) δ 13.58 (br s, 1H), 8.47 (d, J=4.9 Hz, 1H), 7.39 (s, 1H), 7.32-7.23 (m, 2H), 7.11-7.00 (m, 3H), 4.39-4.21 (m, 2H), 2.94-2.72 (m, 2H), 2.02-1.86 (m, 2H), 1.83-1.72 (m, 1H), 1.71-1.60 (m, 1H). 13.60 (br s, 1H), 8.61-8.39 (m, 1H), 7.47-6.98 (m, 6H), 4.47-4.18 (m, 2H), 3.00-2.71 (m, 2H), 2.06-1.89 (m, 2H), 1.86-1.60 (m, 2H); and (*S)-2,2-Difluoro-2′-(4-fluorophenyl)-3′-(1H-pyrazolo[3,4-b]pyridin-4-yl)-4′,5′-dihydro-7′H-spiro[cyclopropane-1,6′-pyrazolo[1,5-a]pyridine] (Example 326).

### Example 326: (*S)-2,2-Difluoro-2′-(4-fluorophenyl)-3′-1H-pyrazolo[3,4-b]pyridin-4-yl)-4′,5′-dihydro-7′H-spiro[cyclopropane-1,6′-pyrazolo[1,5-a]pyridine]

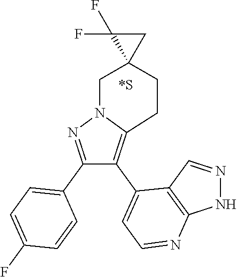 [SMILES: Fc1ccc(-c2nn3c(c2-c2ccnc4[nH]ncc24)CC[C@@]2(C3)CC2(F)F)cc1]

The title compound was isolated from chiral SFC purification of Example 325. MS (ESI): mass calcd. for C₂₁H₁₆F₃N₅ 395.14; m/z found 396.2 [M+H]⁺. ¹H NMR (400 MHz, DMSO-d₆) δ 13.60 (br s, 1H), 8.61-8.39 (m, 1H), 7.47-6.98 (m, 6H), 4.47-4.18 (m, 2H), 3.00-2.71 (m, 2H), 2.06-1.89 (m, 2H), 1.86-1.60 (m, 2H).

### Example 327: (*S)-2,2-Difluoro-2′-(5-fluoropyridin-2-yl)-3′-(1H-pyrazolo[3,4-b]pyridin-4-yl)-4′,5′-dihydro-7′H-spiro[cyclopropane-1,6′-pyrazolo[1,5-a]pyridine]

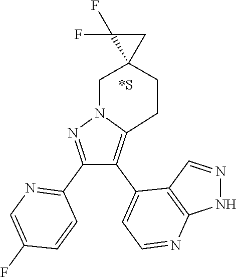 [SMILES: Fc1ccc(-c2nn3c(c2-c2ccnc4[nH]ncc24)CC[C@@]2(C3)CC2(F)F)nc1]

The title compound was prepared in a manner analogous to Example 1, Steps A-B, except using 3′-bromo-2,2-difluoro-2′-(5-fluoropyridin-2-yl)-5′, 7′-dihydro-4′H-spiro[cyclopropane-1,6′-pyrazolo[1,5-a]pyridine] (Intermediate 176) instead of 3-bromo-2-(5-fluoropyridin-2-yl)-6,7-dihydro-4H-pyrazolo[5,1-c][1,4]oxazine (Intermediate 37); and 4-(4,4,5,5-tetramethyl-1,3,2-dioxaborolan-2-yl)-1-((2-(trimethylsilyl)ethoxy)methyl)-1H-pyrazolo[3,4-b]pyridine (Intermediate 23) instead of 4-(4,4,5,5-tetramethyl-1,3,2-dioxaborolan-2-yl)-1-((2-(trimethylsilyl)ethoxy)methyl)-1H-pyrrolo[2,3-b]pyridine (Intermediate 21) in Step A. Purification (chiral HPLC [DAICEL CHIRALCEL OD-H (250 mm*30 mm, 5 um), eluent: 25% to 25% (v/v) EtOH with 0.1% NH₃H₂O)] followed by further purification by HPLC [Phenomenex Gemini-NX C18 75*30 mm*3 um, Mobile Phase A: water (0.05% NH₃H₂O+10 mM NH₄HCO₃), Mobile Phase B: acetonitrile, Flow rate: 25 mL/min, gradient condition from 29% B to 59%)]) afforded the title compound: MS (ESI): mass calcd. for C₂₀H₁₅F₃N₆ 396.1; m/z found 397.1 [M+H]⁺; ¹H NMR (400 MHz, DMSO-d₆): δ 13.50 (br s, 1H), 8.46 (d, J=4.4 Hz, 1H), 8.22 (d, J=2.8 Hz, 1H), 7.92-7.68 (m, 2H), 7.33 (s, 1H), 7.08 (d, J=4.8 Hz, 1H), 4.42-4.27 (m, 2H), 3.02-2.78 (m, 2H), 2.04-1.90 (m, 2H), 1.86-1.61 (m, 2H); and (*R)-2,2-Difluoro-2′-(5-fluoropyridin-2-yl)-3′-(1H-pyrazolo[3,4-b]pyridin-4-yl)-4′,5′-dihydro-7′H-spiro[cyclopropane-1,6′-pyrazolo[1,5-a]pyridine] (Example 328).

### Example 328: (*R)-2,2-Difluoro-2′-(5-fluoropyridin-2-yl)-3′-(1H-pyrazolo[3,4-b]pyridin-4-yl)-4′,5′-dihydro-7′H-spiro[cyclopropane-1,6′-pyrazolo[1,5-a]pyridine]

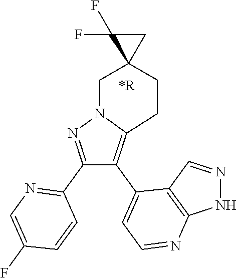 [SMILES: Fc1ccc(-c2nn3c(c2-c2ccnc4[nH]ncc24)CC[C@]2(C3)CC2(F)F)nc1]

The title compound was isolated from chiral SFC purification of Example 327. MS (ESI): mass calcd. for C₂₀H₁₅F₃N₆ 396.1; m/z found 397.0 [M+H]⁺. ¹H NMR (400 MHz, DMSO-d₆): δ 13.50 (br s, 1H), 8.46 (d, J=4.8 Hz, 1H), 8.22 (d, J=2.8 Hz, 1H), 7.86-7.71 (m, 2H), 7.33 (s, 1H), 7.08 (d, J=4.8 Hz, 1H), 4.43-4.26 (m, 2H), 2.97-2.77 (m, 2H), 2.05-1.89 (m, 2H), 1.85-1.64 (m, 2H)

### Example 329: (*S)-2,2-Difluoro-3′-(5-fluoro-1H-pyrazolo[3,4-b]pyridin-4-yl)-2′-(5-fluoropyridin-2-yl)-4′,5′-dihydro-7′H-spiro[cyclopropane-1,6′-pyrazolo[1,5-a]pyridine]

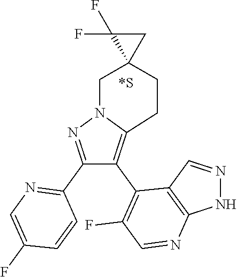 [SMILES: Fc1ccc(-c2nn3c(c2-c2c(F)cnc4[nH]ncc24)CC[C@@]2(C3)CC2(F)F)nc1]

Step A: 2,2-Difluoro-3′-(5-fluoro-1-(tetrahydro-2H-pyran-2-yl)-1H-pyrazolo[3,4-b]pyridin-4-yl)-2′-(5-fluoropyridin-2-yl)-4′,5′-dihydro-7′H-spiro[cyclopropane-1,6′-pyrazolo[1,5-a]pyridine]. 3′-Bromo-2,2-difluoro-2′-(5-fluoropyridin-2-yl)-5′,7′-dihydro-4′H-spiro[cyclopropane-1,6′-pyrazolo[1,5-a]pyridine] (Intermediate 184, 240 mg, 0.536 mmol), 5-fluoro-4-iodo-1-(tetrahydro-2H-pyran-2-yl)-1H-pyrazolo[3,4-b]pyridine (Intermediate 230, 220 mg, 0.634 mmol), and 4,4,4′,4′,5,5,5′,5′-octamethyl-2,2′-bi(1,3,2-dioxaborolane) (220 mg, 0.866 mmol), and Cs₂CO₃ (528 mg, 1.62 mmol) were added to a 10 mL microwave tube and the resulting mixture dissolved in 1,4-dioxane (3 mL) and H₂O (0.5 mL). The resultant mixture was sparged with Ar for 5 minutes and then treated with CataCXium® A-Pd-G3 (77.0 mg, 0.106 mmol). The resultant mixture was sparged with Ar for another 3 minutes and heated at 100° C. via microwave irradiation for 2 hours then cooled to room-temperature. The reaction mixture was poured into H₂O (30 mL) and extracted with dichloromethane (30 mL×3). The combined organic extracts were dried over anhydrous Na₂SO₄, filtered, and concentrated to dryness under reduced pressure. The resulting residue was purified by FCC (SiO₂, eluent: petroleum ether:ethyl acetate=1:0 to 1:1) to afford the title compound (50.0 mg, 18%) as a yellow solid. MS (ESI): mass calcd. for C₂₅H₂₂F₄N₆O 498.2; m/z found 499.1 [M+H]⁺.

Step B: 2,2-Difluoro-3′-(5-fluoro-1H-pyrazolo[3,4-b]pyridin-4-yl)-2′-(5-fluoropyridin-2-yl)-4′,5′-dihydro-7′H-spiro[cyclopropane-1,6′-pyrazolo[1,5-a]pyridine]. HCl/1,4-dioxane (2 mL, 8 mmol, 4 M) was added to a solution consisting of 2,2-difluoro-3′-(5-fluoro-1-(tetrahydro-2H-pyran-2-yl)-1H-pyrazolo[3,4-b]pyridin-4-yl)-2′-(5-fluoropyridin-2-yl)-5′,7′-dihydro-4′H-spiro[cyclopropane-1,6′-pyrazolo[1,5-a]pyridine] (50.0 mg, 0.100 mmol) and dichloromethane (3 mL). The mixture was stirred at room-temperature for 6 hours. The reaction mixture was concentrated to dryness under reduced pressure to give a residue, which was diluted with MeCN (1 mL), basified with aq. NH₃H₂O (25%) to pH=9. The mixture was combined with an earlier batch and purified by reverse phase silica gel column (Agela C18, 4 g) using water and acetonitrile as eluents (Mobile phase A water, Mobile phase B acetonitrile, Mobile phase B from 35% to 65%) to afford the title compound (40.0 mg) as a white solid. MS (ESI): mass calcd. for C₂₀H₁₄F₄N₆ 414.1 m/z found; 415.1 [M+H]⁺. Purification (SFC over DAICEL CHIRALPAK AD 250 mm×30 mm, 10 μm (isocratic elution: EtOH (containing 0.1% of 25% aq. NH₃): supercritical CO₂, 80%: 20% to 80%: 20% (v/v)) and concentration of the desired fractions afforded the title compound (6.1 mg, 15% yield) as a white powder: MS (ESI): mass calcd. for C₂₀H₁₄F₄N₆ 414.1; m/z found 415.0 [M+H]⁺; ¹H NMR (400 MHz, DMSO-d₆) δ 13.92-13.62 (m, 1H), 8.58-8.48 (m, 1H), 8.24-8.16 (m, 1H), 7.98-7.89 (m, 1H), 7.81-7.62 (m, 2H), 4.50-4.26 (m, 2H), 2.94-2.58 (m, 2H), 2.12-1.64 (m, 4H); and (*R)-2,2-Difluoro-3′-(5-fluoro-1H-pyrazolo[3,4-b]pyridin-4-yl)-2′-(5-fluoropyridin-2-yl)-4′,5′-dihydro-7′H-spiro[cyclopropane-1,6′-pyrazolo[1,5-a]pyridine] (Example 330) (4.2 mg, 10% yield) as a white powder.

### Example 330: (*R)-2,2-Difluoro-3′-(5-fluoro-1H-pyrazolo[3,4-b]pyridin-4-yl)-2′-(5-fluoropyridin-2-yl)-4′,5′-dihydro-7′H-spiro[cyclopropane-1,6′-pyrazolo[1,5-a]pyridine]

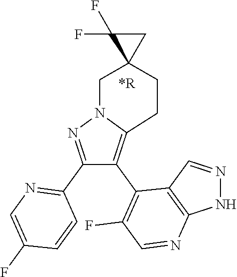 [SMILES: Fc1ccc(-c2nn3c(c2-c2c(F)cnc4[nH]ncc24)CC[C@]2(C3)CC2(F)F)nc1]

The title compound was isolated from chiral SFC purification of Example 329, Step B. MS (ESI): mass calcd. for C₂₀H₁₄F₄N₆ 414.1; m/z found 415.0 [M+H]⁺. ¹H NMR (400 MHz, DMSO-d₆): δ 13.74 (br s, 1H), 8.58-8.48 (m, 1H), 8.25-8.16 (m, 1H), 7.97-7.89 (m, 1H), 7.80-7.61 (m, 2H), 4.55-4.22 (m, 2H), 2.95-2.58 (m, 2H), 2.22-1.58 (m, 4H).

### Example 331: (*S)-2,2-Difluoro-3′-(5-fluoro-6-methyl-1H-pyrazolo[3,4-b]pyridin-4-yl)-2′-(5-fluoropyridin-2-yl)-4′,5′-dihydro-7′H-spiro[cyclopropane-1,6′-pyrazolo[1,5-a]pyridine]

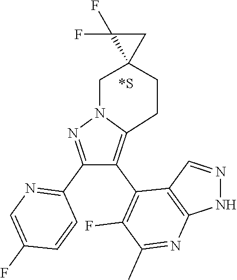 [SMILES: Cc1nc2[nH]ncc2c(-c2c(-c3ccc(F)cn3)nn3c2CC[C@@]2(C3)CC2(F)F)c1F]

The title compound was prepared in a manner analogous to Example 329, Steps A, B and C, except using 5-fluoro-4-iodo-6-methyl-1-(tetrahydro-2H-pyran-2-yl)-1H-pyrazolo[3,4-b]pyridine (Intermediate 215) instead of 5-fluoro-4-iodo-1-(tetrahydro-2H-pyran-2-yl)-1H-pyrazolo[3,4-b]pyridine (Intermediate 230) in Step A. MS (ESI): mass calcd. for C₂₁H₁₆F₄N₆ 428.1; m/z found 429.1 [M+H]⁺. ¹H NMR (400 MHz, CD₃OD-d₄) δ 8.27-8.09 (m, 1H), 7.89-7.76 (m, 1H), 7.68-7.54 (m, 1H), 7.51-7.39 (m, 1H), 4.51-4.25 (m, 2H), 2.97-2.75 (m, 2H), 2.67-2.60 (m, 3H), 2.21-1.92 (m, 2H), 1.72-1.55 (m, 2H). N—H proton not observed.

### Example 332: (*R)-2,2-Difluoro-3′-(5-fluoro-6-methyl-1H-pyrazolo[3,4-b]pyridin-4-yl)-2′-(5-fluoropyridin-2-yl)-4′,5′-dihydro-7′H-spiro[cyclopropane-1,6′-pyrazolo[1,5-a]pyridine]

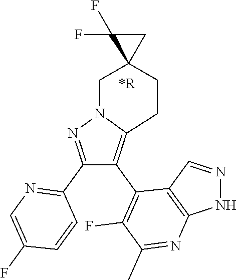 [SMILES: Cc1nc2[nH]ncc2c(-c2c(-c3ccc(F)cn3)nn3c2CC[C@]2(C3)CC2(F)F)c1F]

The title compound was prepared in a manner analogous to Example 329, Steps A, B and C, except using 5-fluoro-4-iodo-6-methyl-1-(tetrahydro-2H-pyran-2-yl)-1H-pyrazolo[3,4-b]pyridine (Intermediate 215) instead of 5-fluoro-4-iodo-1-(tetrahydro-2H-pyran-2-yl)-1H-pyrazolo[3,4-b]pyridine (Intermediate 230) in Step A. MS (ESI): mass calcd. for C₂₁H₁₆F₄N₆ 428.1; m/z found 429.1 [M+H]⁺. ¹H NMR (400 MHz, CD₃OD-d4) δ 8.28-8.10 (m, 1H), 7.90-7.70 (m, 1H), 7.66-7.52 (m, 1H), 7.48-7.37 (m, 1H), 4.50-4.24 (m, 2H), 2.96-2.71 (m, 2H), 2.68-2.55 (m, 3H), 2.18-1.95 (m, 2H), 1.71-1.52 (m, 2H). N—H proton not observed.

### Example 333: (*S)-2,2-Difluoro-3′-(5-fluoro-3,6-dimethyl-1H-pyrazolo[3,4-b]pyridin-4-yl)-2′-(5-fluoropyridin-2-yl)-4′,5′-dihydro-7′H-spiro[cyclopropane-1,6′-pyrazolo[1,5-a]pyridine]

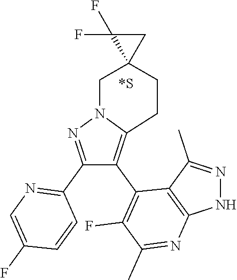 [SMILES: Cc1nc2[nH]nc(C)c2c(-c2c(-c3ccc(F)cn3)nn3c2CC[C@@]2(C3)CC2(F)F)c1F]

The title compound was prepared in a manner similar to Example 329, Steps A, B and C, except using 4-bromo-5-fluoro-3,6-dimethyl-1-((2-(trimethylsilyl)ethoxy)methyl)-1H-pyrazolo[3,4-b]pyridine (Intermediate 209) instead of 5-Fluoro-4-iodo-1-(tetrahydro-2H-pyran-2-yl)-1H-pyrazolo[3,4-b]pyridine (Intermediate 230) in Step A. Alternative purification of SFC method: (DAICEL CHIRALPAK AD (250 mm*30 mm, 10 um), Condition:Mobile phase: A: CO₂; Mobile phase: B: 0.1% NH₃H₂O ETOH, Flow rate: 70 mL/min, gradient condition from 55% B to 55%) was employed in Step C. MS (ESI): mass calcd. for C₂₂H₁₈F₄N₆ 442.2; m/z found 443.2 [M+H]⁺. ¹H NMR (400 MHz, DMSO-d₆) δ 13.11 (s, 1H), 8.15 (d, J=2.8 Hz, 1H), 7.99-7.91 (m, 1H), 7.77-7.68 (m, 1H), 4.49-4.28 (m, 2H), 2.74-2.61 (m, 1H), 2.54 (d, J=3.6 Hz, 3H), 2.06-1.90 (m, 2H), 1.86 (s, 4H), 1.75-1.64 (m, 1H), 1.30-1.17 (m, 1H).

### Example 334: (*R)-2,2-Difluoro-3′-(5-fluoro-3,6-dimethyl-1H-pyrazolo[3,4-b]pyridin-4-yl)-2′-(5-fluoropyridin-2-yl)-4′,5′-dihydro-7′H-spiro[cyclopropane-1,6′-pyrazolo[1,5-a]pyridine]

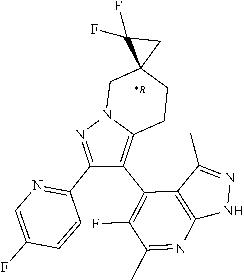 [SMILES: Cc1nc2[nH]nc(C)c2c(-c2c(-c3ccc(F)cn3)nn3c2CC[C@]2(C3)CC2(F)F)c1F]

The title compound was prepared in a manner similar to Example 329, Steps A, B and C, except using 4-bromo-5-fluoro-3,6-dimethyl-1-((2-(trimethylsilyl)ethoxy)methyl)-1H-pyrazolo[3,4-b]pyridine (Intermediate 209) instead of 5-Fluoro-4-iodo-1-(tetrahydro-2H-pyran-2-yl)-1H-pyrazolo[3,4-b]pyridine (Intermediate 230) in Step A. Alternative purification of SFC method: (DAICEL CHIRALPAK AD (250 mm*30 mm, 10 um), Condition:Mobile phase: A: CO₂; Mobile phase: B: 0.1% NH₃H₂O ETOH, Flow rate: 70 mL/min, gradient condition from 55% B to 55%) was employed in Step C. MS (ESI): mass calcd. for C₂₂H₁₈F₄N₆ 442.2; m/z found 443.2 [M+H]⁺. ¹H NMR (400 MHz, DMSO-d₆) δ13.11 (s, 1H), 8.15 (d, J=3.2 Hz, 1H), 7.99-7.92 (m, 1H), 7.76-7.67 (m, 1H), 4.49-4.27 (m, 2H), 2.76-2.61 (m, 1H), 2.54 (d, J=3.6 Hz, 3H), 2.08-1.89 (m, 2H), 1.88-1.78 (m, 4H), 1.75-1.65 (m, 1H), 1.29-1.19 (m, 1H).

### Example 335: (*S)-2,2-Difluoro-2′-(4-fluorophenyl)-3′-(6-methyl-1H-pyrazolo[3,4-d]pyrimidin-4-yl)-4′,5′-dihydro-7′H-spiro[cyclopropane-1,6′-pyrazolo[1,5-a]pyridine]

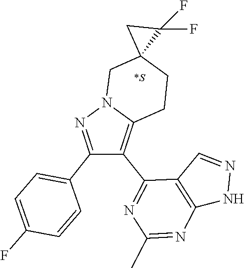 [SMILES: Cc1nc(-c2c(-c3ccc(F)cc3)nn3c2CC[C@@]2(C3)CC2(F)F)c2cn[nH]c2n1]

The title compound was prepared in a manner analogous Example 329, Steps A, B and C, except using 3′-bromo-2,2-difluoro-2′-(4-fluorophenyl)-5′,7′-dihydro-4′H-spiro[cyclopropane-1,6′-pyrazolo[1,5-a]pyridine] (Intermediate 233) instead of 3′-bromo-2,2-difluoro-2′-(5-fluoropyridin-2-yl)-5′,7′-dihydro-4′H-spiro[cyclopropane-1,6′-pyrazolo[1,5-a]pyridine] (Intermediate 184) and 4-chloro-6-methyl-1-((2-(trimethylsilyl)ethoxy)methyl)-1H-pyrazolo[3,4-d]pyrimidine (Intermediate 185) instead of 5-fluoro-4-iodo-1-(tetrahydro-2H-pyran-2-yl)-1H-pyrazolo[3,4-b]pyridine (Intermediate 230) in Step A. Alternative purification of SFC over DAICEL CHIRALPAK AD (250 mm*30 mm, 10 um) (isocratic elution: EtOH (containing 0.1% of 25% aq. NH₃): supercritical CO₂, 15%: 85% to 15%: 85% (v/v)) was used for Step C. MS (ESI): mass calcd. for C₂₁H₁₇F₃N₆ 410.2; m/z found 411.2 [M+H]⁺. ¹H NMR (400 MHz, DMSO-d₆) δ 13.64 (s, 1H), 7.42-7.31 (m, 2H), 7.19-7.09 (m, 2H), 7.02 (s, 1H), 4.38-4.25 (m, 2H), 3.17-2.97 (m, 2H), 2.69 (s, 3H), 2.05-1.91 (m, 2H), 1.85-1.63 (m, 2H).

### Example 336: (*R)-2,2-Difluoro-2′-(4-fluorophenyl)-3′-(6-methyl-1H-pyrazolo[3,4-d]pyrimidin-4-yl)-4′,5′-dihydro-7′H-spiro[cyclopropane-1,6′-pyrazolo[1,5-a]pyridine]

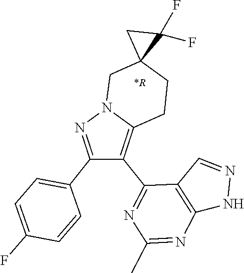 [SMILES: Cc1nc(-c2c(-c3ccc(F)cc3)nn3c2CC[C@]2(C3)CC2(F)F)c2cn[nH]c2n1]

The title compound was prepared in a manner analogous Example 329, Steps A, B and C, except using 3′-bromo-2,2-difluoro-2′-(4-fluorophenyl)-5′,7′-dihydro-4′H-spiro[cyclopropane-1,6′-pyrazolo[1,5-a]pyridine] (Intermediate 233) instead of 3′-bromo-2,2-difluoro-2′-(5-fluoropyridin-2-yl)-5′,7′-dihydro-4′H-spiro[cyclopropane-1,6′-pyrazolo[1,5-a]pyridine] (Intermediate 184) and 4-chloro-6-methyl-1-((2-(trimethylsilyl)ethoxy)methyl)-1H-pyrazolo[3,4-d]pyrimidine (Intermediate 185) instead of 5-Fluoro-4-iodo-1-(tetrahydro-2H-pyran-2-yl)-1H-pyrazolo[3,4-b]pyridine (Intermediate 230) in Step A. Alternative purification of SFC over DAICEL CHIRALPAK AD (250 mm*30 mm, 10 um) (isocratic elution: EtOH (containing 0.1% of 25% aq. NH₃): supercritical CO₂, 15%: 85% to 15%: 85% (v/v)) was used for Step C. MS (ESI): mass calcd. for C₂₁H₁₇F₃N₆ 410.2; m/z found 411.2 [M+H]⁺. ¹H NMR (400 MHz, DMSO-d₆) δ 13.61 (br s, 1H), 7.44-7.32 (m, 2H), 7.19-7.10 (m, 2H), 7.02 (s, 1H), 4.40-4.23 (m, 2H), 3.15-2.97 (m, 2H), 2.69 (s, 3H), 2.06-1.90 (m, 2H), 1.85-1.62 (m, 2H).

### Example 337: (1*S,4′*S)-4′-Chloro-2,2-difluoro-2′-(5-fluoropyridin-2-yl)-3′-(1H-pyrazolo[3,4-b]pyridin-4-yl)-4′,5′-dihydro-7′H-spiro[cyclopropane-1,6′-pyrazolo[1,5-a]pyridine]

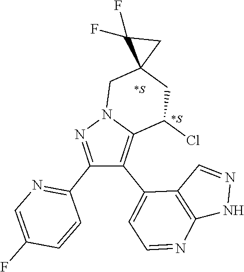 [SMILES: Fc1ccc(-c2nn3c(c2-c2ccnc4[nH]ncc24)[C@@H](Cl)C[C@]2(C3)CC2(F)F)nc1]

To a solution of (*R)-2,2-difluoro-2′-(5-fluoropyridin-2-yl)-3′-(1H-pyrazolo[3,4-b]pyridin-4-yl)-4′,5′-dihydro-7′H-spiro[cyclopropane-1,6′-pyrazolo[1,5-a]pyridine] (Example 328, 74 mg, 0.187 mol) in DMF (2 mL) was added NCS (26 mg, 0.195 mmol). The reaction mixture was stirred at room temperature for 17 hr. The title compound and (1*S,4′*R)-4′-chloro-2,2-difluoro-2′-(5-fluoropyridin-2-yl)-3′-(1H-pyrazolo[3,4-b]pyridin-4-yl)-4′,5′-dihydro-7′H-spiro[cyclopropane-1,6′-pyrazolo[1,5-a]pyridine] (Example 338) was purified (Prep-HPLC (Phenomenex Gemini-NX 80*40 mm*3 um, Mobile Phase A: water (0.05% NH₃/H₂O), Mobile Phase B: acetonitrile, Flow rate: 30 mL/min, gradient condition from 29% B to 59%)); and further purified by Prep-HPLC (Boston Green ODS 150*30 mm*5 um, Mobile Phase A: water (0.225% FA), Mobile Phase B: acetonitrile, Flow rate: 35 mL/min, gradient condition from 35% B to 65%)) and concentrated to afford the title compound (4 mg, yield: 4%) as a white solid. MS (ESI): mass calcd. for C₂₀H₁₄ClF₃N₆ 430.1; m/z found 431.1 [M+H]⁺. ¹H NMR (400 MHz, DMSO-d₆): δ 13.57 (s, 1H), 8.54 (d, J=5.2 Hz, 1H), 8.20 (d, J=2.8 Hz, 1H), 7.89-7.84 (m, 1H), 7.80-7.74 (m, 1H), 7.38-7.29 (m, 2H), 5.79 (br s, 1H), 4.60 (d, J=13.6 Hz, 1H), 4.37 (d, J=13.2 Hz, 1H), 2.95-2.88 (m, 1H), 2.23-2.14 (m, 1H), 2.08 (d, J=15.2 Hz, 1H), 1.90-1.82 (m, 1H).

### Example 338: (1*S,4′*R)-4′-Chloro-2,2-difluoro-2′-(5-fluoropyridin-2-yl)-3′-(1H-pyrazolo[3,4-b]pyridin-4-yl)-4′,5′-dihydro-7′H-spiro[cyclopropane-1,6′-pyrazolo[1,5-a]pyridine]

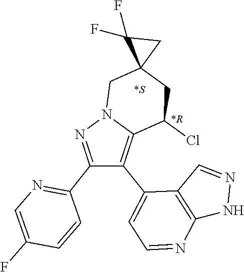 [SMILES: Fc1ccc(-c2nn3c(c2-c2ccnc4[nH]ncc24)[C@H](Cl)C[C@]2(C3)CC2(F)F)nc1]

The title compound was purified from Example 337; (Prep-HPLC (Welch Xtimate C18 150*25 mm*5 um, Mobile Phase A: water (0.225% FA), Mobile Phase B: acetonitrile, Flow rate: 25 mL/min, gradient condition from 37% B to 67%)) to afford the title compound (6 mg, 7%) as a white solid. MS (ESI): mass calcd. for C₂₀H₁₄ClF₃N₆ 430.1 m/z found 431.1 [M+H]⁺. ¹H NMR (400 MHz, DMSO-d₆): δ 13.56 (s, 1H), 8.53 (d, J=4.8 Hz, 1H), 8.20 (d, J=2.7 Hz, 1H), 7.91-7.71 (m, 2H), 7.46-7.22 (m, 2H), 5.71 (br s, 1H), 4.59-4.25 (m, 2H), 3.02-2.85 (m, 1H), 2.17 (br d, J=13.7 Hz, 1H), 1.89-1.72 (m, 2H).

### Example 339: (5a*S,6a*R)-2-(4-Fluorophenyl)-3-(1H-pyrazolo[3,4-b]pyridin-4-yl)-5,5a,6,6a-tetrahydro-4H-cyclopropa[e]pyrazolo[1,5-a]pyridine

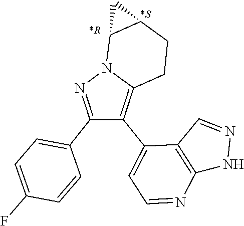 [SMILES: Fc1ccc(-c2nn3c(c2-c2ccnc4[nH]ncc24)CC[C@H]2C[C@H]23)cc1]

The title compound was prepared in a manner analogous to 4-(6,6-difluoro-2-(4-fluorophenyl)-4,5,6,7-tetrahydropyrazolo[1,5-a]pyridin-3-yl)-5-fluoro-1H-pyrazolo[3,4-b]pyridine (Example 280), except using 4-(4,4,5,5-tetramethyl-1,3,2-dioxaborolan-2-yl)-1-((2-(trimethylsilyl)ethoxy)methyl)-1H-pyrazolo[3,4-b]pyridine (Intermediate 23) instead of 5-fluoro-4-(4,4,5,5-tetramethyl-1,3,2-dioxaborolan-2-yl)-1-((2-(trimethylsilyl)ethoxy)methyl)-1H-pyrazolo[3,4-b]pyridine (Intermediate 222) and racemic 3-bromo-2-(4-fluorophenyl)-5,5a,6,6a-tetrahydro-4H-cyclopropa[e]pyrazolo[1,5-a]pyridine (Intermediate 178) instead of 3-bromo-6,6-difluoro-2-(4-fluorophenyl)-4,5,6,7-tetrahydropyrazolo[1,5-a]pyridine (Intermediate 52). The title compound was separated from its enantiomer using chiral SFC (Stationary phase: Chiralpak AD3 Sum 250×21 mm, Mobile phase: 20% methanol, 80% CO₂). MS (ESI): mass calcd. for C₂₀H₁₆FN₅ 345.1; m/z found 346.1 [M+H]⁺. ¹H NMR (600 MHz, CDCl₃): δ 12.19 (s, 1H), 8.55 (d, J=4.8 Hz, 1H), 7.56 (s, 1H), 7.39-7.33 (m, 2H), 6.94 (d, J=4.8 Hz, 1H), 6.93-6.89 (m, 2H), 3.99-3.87 (m, 1H), 2.88-2.77 (m, 1H), 2.59-2.48 (m, 1H), 2.13-2.06 (m, 2H), 1.81-1.73 (m, 1H), 1.24-1.15 (m, 1H), 1.06-0.97 (m, 1H).

### Example 340: (5a*R,6a*S)-2-(4-Fluorophenyl)-3-(1H-pyrazolo[3,4-b]pyridin-4-yl)-5,5a,6,6a-tetrahydro-4H-cyclopropa[e]pyrazolo[1,5-a]pyridine

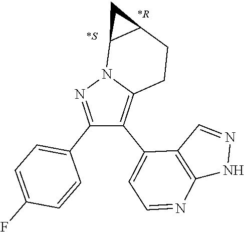 [SMILES: Fc1ccc(-c2nn3c(c2-c2ccnc4[nH]ncc24)CC[C@@H]2C[C@@H]23)cc1]

The title compound was prepared in a manner analogous to 4-(6,6-difluoro-2-(4-fluorophenyl)-4,5,6,7-tetrahydropyrazolo[1,5-a]pyridin-3-yl)-5-fluoro-1H-pyrazolo[3,4-b]pyridine (Example 281), except using 4-(4,4,5,5-tetramethyl-1,3,2-dioxaborolan-2-yl)-1-((2-(trimethylsilyl)ethoxy)methyl)-1H-pyrazolo[3,4-b]pyridine (Intermediate 23) instead of 5-fluoro-4-(4,4,5,5-tetramethyl-1,3,2-dioxaborolan-2-yl)-1-((2-(trimethylsilyl)ethoxy)methyl)-1H-pyrazolo[3,4-b]pyridine (Intermediate 222) and racemic 3-bromo-2-(4-fluorophenyl)-5,5a,6,6a-tetrahydro-4H-cyclopropa[e]pyrazolo[1,5-a]pyridine (Intermediate 178) instead of 3-bromo-6,6-difluoro-2-(4-fluorophenyl)-4,5,6,7-tetrahydropyrazolo[1,5-a]pyridine (Intermediate 52). The title compound was separated from its enantiomer using chiral SFC (Stationary phase: Chiralpak AD3 Sum 250×21 mm, Mobile phase: 20% methanol, 80% CO₂). MS (ESI): mass calcd. for C₂₀H₁₆FN₅ 345.1; m/z found 346.2 [M+H]⁺. ¹H NMR (600 MHz, CDCl₃): δ 12.10 (s, 1H), 8.55 (d, J=4.8 Hz, 1H), 7.55 (s, 1H), 7.39-7.33 (m, 2H), 6.97-6.88 (m, 3H), 4.02-3.88 (m, 1H), 2.91-2.80 (m, 1H), 2.63-2.46 (m, 1H), 2.17-2.04 (m, 2H), 1.81-1.73 (m, 1H), 1.30-1.13 (m, 1H), 1.07-0.94 (m, 1H).

### Example 341: N-(4-((5a*R,6a*S)-2-(5-Fluoropyridin-2-yl)-5,5a,6,6a-tetrahydro-4H-cyclopropa[e]pyrazolo[1,5-a]pyridin-3-yl)pyridin-2-yl)acetamide

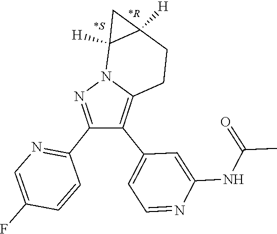 [SMILES: CC(=O)Nc1cc(-c2c(-c3ccc(F)cn3)nn3c2CC[C@@H]2C[C@@H]23)ccn1]

The title compound was prepared in a manner analogous to (5a*R,6a*S)-6,6-difluoro-2-(4-fluorophenyl)-3-(pyrazolo[1,5-a]pyridin-4-yl)-5,5a,6,6a-tetrahydro-4H-cyclopropa[e]pyrazolo[1,5-a]pyridine (Example 354), except that (5a*R,6a*S)-3-bromo-2-(5-fluoropyridin-2-yl)-5,5a,6,6a-tetrahydro-4H-cyclopropa[e]pyrazolo[1,5-a]pyridine (Intermediate 179) was used instead of racemic 3-bromo-6,6-difluoro-2-(4-fluorophenyl)-5,5a,6,6a-tetrahydro-4H-cyclopropa[e]pyrazolo[1,5-a]pyridine (Intermediate 181), N-(4-(4,4,5,5-tetramethyl-1,3,2-dioxaborolan-2-yl)pyridin-2-yl)acetamide was used instead of pyrazolo[1,5-a]pyridin-5-ylboronic acid, and no chiral separation was performed. MS (ESI): mass calcd. for C₂₀H₁₈FN₅O 363.2; m/z found 364.2 [M+H]⁺. ¹H NMR (400 MHz, CDCl₃): δ 8.43-8.37 (m, 1H), 8.17-8.08 (m, 2H), 7.88 (s, 1H), 7.63-7.55 (m, 1H), 7.39-7.30 (m, 1H), 6.88-6.79 (m, 1H), 4.01-3.83 (m, 1H), 3.00-2.85 (m, 1H), 2.69-2.48 (m, 1H), 2.19 (s, 3H), 2.17-1.96 (m, 2H), 1.82-1.65 (m, 1H), 1.22-1.10 (m, 1H), 1.03-0.91 (m, 1H).

### Example 342: N-(4-((5a*S,6a*R)-2-(5-Fluoropyridin-2-yl)-5,5a,6,6a-tetrahydro-4H-cyclopropa[e]pyrazolo[1,5-a]pyridin-3-yl)pyridin-2-yl)acetamide

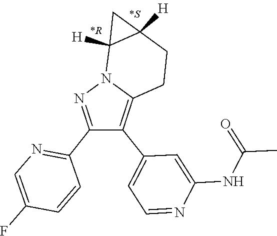 [SMILES: CC(=O)Nc1cc(-c2c(-c3ccc(F)cn3)nn3c2CC[C@H]2C[C@H]23)ccn1]

The title compound was prepared in a manner analogous to (5a*R,6a*S)-6,6-difluoro-2-(4-fluorophenyl)-3-(pyrazolo[1,5-a]pyridin-4-yl)-5,5a,6,6a-tetrahydro-4H-cyclopropa[e]pyrazolo[1,5-a]pyridine (Example 354), except that (5a*S,6a*R)-3-bromo-2-(5-fluoropyridin-2-yl)-5,5a,6,6a-tetrahydro-4H-cyclopropa[e]pyrazolo[1,5-a]pyridine (Intermediate 180) was used instead of racemic 3-bromo-6,6-difluoro-2-(4-fluorophenyl)-5,5a,6,6a-tetrahydro-4H-cyclopropa[e]pyrazolo[1,5-a]pyridine (Intermediate 181), N-(4-(4,4,5,5-tetramethyl-1,3,2-dioxaborolan-2-yl)pyridin-2-yl)acetamide was used instead of pyrazolo[1,5-a]pyridin-5-ylboronic acid, and no chiral separation was performed. MS (ESI): mass calcd. for C₂₀H₁₈FN₅O 363.2; m/z found 364.2 [M+H]⁺. ¹H NMR (400 MHz, CDCl₃): δ 8.40 (d, J=3.0 Hz, 1H), 8.16-8.03 (m, 2H), 7.88 (s, 1H), 7.64-7.57 (m, 1H), 7.41-7.31 (m, 1H), 6.83 (dd, J=5.2, 1.6 Hz, 1H), 3.97-3.84 (m, 1H), 2.97-2.87 (m, 1H), 2.68-2.52 (m, 1H), 2.19 (s, 3H), 2.16-1.97 (m, 2H), 1.81-1.69 (m, 1H), 1.20-1.10 (m, 1H), 1.02-0.95 (m, 1H).

### Example 343: (5a*R,6a*S)-3-(5-Fluoro-1H-pyrazolo[3,4-b]pyridin-4-yl)-2-(5-fluoropyridin-2-yl)-5,5a,6,6a-tetrahydro-4H-cyclopropa[e]pyrazolo[1,5-a]pyridine

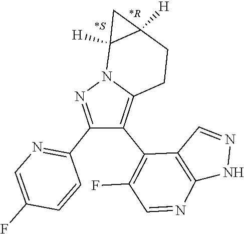 [SMILES: Fc1ccc(-c2nn3c(c2-c2c(F)cnc4[nH]ncc24)CC[C@@H]2C[C@@H]23)nc1]

The title compound was prepared in a manner analogous to (5a*S,6a*R)-6,6-difluoro-3-(5-fluoro-1H-pyrazolo[3,4-b]pyridin-4-yl)-2-(5-fluoropyridin-2-yl)-5,5a,6,6a-tetrahydro-4H-cyclopropa[e]pyrazolo[1,5-a]pyridine (Example 358) except using (5a*R,6a*S)-3-bromo-2-(5-fluoropyridin-2-yl)-5,5a,6,6a-tetrahydro-4H-cyclopropa[e]pyrazolo[1,5-a]pyridine (Intermediate 179) instead of (5a*S,6a*R)-6,6-difluoro-3-bromo-2-(5-fluoropyridin-2-yl)-5,5a,6,6a-tetrahydro-4H-cyclopropa[e]pyrazolo[1,5-a]pyridine (Intermediate 182). MS (ESI): mass calcd. for C₁₉H₁₄F₂N₆ 364.1; m/z found 365.2 [M+H]⁺. ¹H NMR (400 MHz, CDCl₃): δ 11.71 (s, 1H), 8.49 (dd, J=10.9, 2.7 Hz, 1H), 8.17 (d, J=2.8 Hz, 1H), 7.77-7.64 (m, 1H), 7.54 (d, J=47.9 Hz, 1H), 7.40-7.29 (m, 1H), 4.04-3.87 (m, 1H), 2.80-2.62 (m, 1H), 2.61-2.35 (m, 1H), 2.24-1.97 (m, 2H), 1.82-1.74 (m, 1H), 1.24-1.15 (m, 1H), 1.12-1.00 (m, 1H).

### Example 344: (5a*R,6a*S)-3-(5-Fluoro-6-methyl-1H-pyrazolo[3,4-b]pyridin-4-yl)-2-(5-fluoropyridin-2-yl)-5,5a,6,6a-tetrahydro-4H-cyclopropa[e]pyrazolo[1,5-a]pyridine

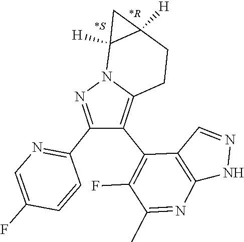 [SMILES: Cc1nc2[nH]ncc2c(-c2c(-c3ccc(F)cn3)nn3c2CC[C@@H]2C[C@@H]23)c1F]

The title compound was prepared in a manner analogous to (5a*S,6a*R)-6,6-difluoro-3-(5-fluoro-1H-pyrazolo[3,4-b]pyridin-4-yl)-2-(5-fluoropyridin-2-yl)-5,5a,6,6a-tetrahydro-4H-cyclopropa[e]pyrazolo[1,5-a]pyridine (Example 358) except using (5a*R,6a*S)-3-bromo-2-(5-fluoropyridin-2-yl)-5,5a,6,6a-tetrahydro-4H-cyclopropa[e]pyrazolo[1,5-a]pyridine (Intermediate 179) instead of (5a*S,6a*R)-6,6-difluoro-3-bromo-2-(5-fluoropyridin-2-yl)-5,5a,6,6a-tetrahydro-4H-cyclopropa[e]pyrazolo[1,5-a]pyridine (Intermediate 182), 5-fluoro-6-methyl-1-(tetrahydro-2H-pyran-2-yl)-4-(tributylstannyl)-1H-pyrazolo[3,4-b]pyridine (Intermediate 237) instead of 5-fluoro-4-(tributylstannyl)-1-((2-(trimethylsilyl)ethoxy)methyl)-1H-pyrazolo[3,4-b]pyridine (Intermediate 235), and 4N HCl in dioxane instead of TFA. MS (ESI): mass calcd. for C₂₀H₁₆F₂N₆ 378.1; m/z; found 379.2 [M+H]⁺. ¹H NMR (400 MHz, CDCl₃): δ 10.63 (s, 1H), 8.18 (d, J=2.9 Hz, 1H), 7.69 (td, J=8.3, 4.4 Hz, 1H), 7.38-7.29 (m, 2H), 4.06-3.83 (m, 1H), 2.88-2.33 (m, 5H), 2.22-1.94 (m, 2H), 1.77 (s, 1H), 1.27-1.13 (m, 1H), 1.13-0.95 (m, 1H).

### Example 345: (5a*R,6a*S)-3-(5-Fluoro-3-methyl-1H-pyrazolo[3,4-b]pyridin-4-yl)-2-(5-fluoropyridin-2-yl)-5,5a,6,6a-tetrahydro-4H-cyclopropa[e]pyrazolo[1,5-a]pyridine

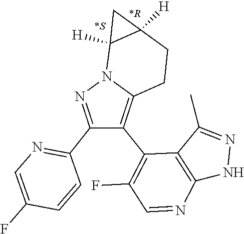 [SMILES: Cc1n[nH]c2ncc(F)c(-c3c(-c4ccc(F)cn4)nn4c3CC[C@@H]3C[C@@H]34)c12]

The title compound was prepared in a manner analogous to (5a*S,6a*R)-6,6-difluoro-3-(5-fluoro-1H-pyrazolo[3,4-b]pyridin-4-yl)-2-(5-fluoropyridin-2-yl)-5,5a,6,6a-tetrahydro-4H-cyclopropa[e]pyrazolo[1,5-a]pyridine (Example 358) except using (5a*R,6a*S)-3-bromo-2-(5-fluoropyridin-2-yl)-5,5a,6,6a-tetrahydro-4H-cyclopropa[e]pyrazolo[1,5-a]pyridine (Intermediate 179) instead of (5a*S,6a*R)-6,6-difluoro-3-bromo-2-(5-fluoropyridin-2-yl)-5,5a,6,6a-tetrahydro-4H-cyclopropa[e]pyrazolo[1,5-a]pyridine (Intermediate 182), 5-fluoro-3-methyl-1-(tetrahydro-2H-pyran-2-yl)-4-(tributylstannyl)-1H-pyrazolo[3,4-b]pyridine (Intermediate 238) instead of 5-fluoro-4-(tributylstannyl)-1-((2-(trimethylsilyl)ethoxy)methyl)-1H-pyrazolo[3,4-b]pyridine (Intermediate 235), and 4N HCl in dioxane instead of TFA. MS (ESI): mass calcd. for C₂₀H₁₆F₂N₆, 378.1; m/z found, 379.2 [M+H]⁺. ¹H NMR (400 MHz, CDCl₃): δ 8.43 (s, 1H), 7.95 (d, J=3.0 Hz, 1H), 7.84 (dd, J=8.7, 4.5 Hz, 1H), 7.38-7.25 (m, 1H), 4.02-3.70 (m, 2H), 2.25 (dd, J=8.5, 0.8 Hz, 2H), 2.22-2.06 (m, 1H), 2.01 (s, 3H), 1.72 (s, 2H), 1.25-0.68 (m, 2H).

### Example 346: (5a*R,6a*S)-3-(5-Fluoro-3,6-dimethyl-1H-pyrazolo[3,4-b]pyridin-4-yl)-2-(5-fluoropyridin-2-yl)-5,5a,6,6a-tetrahydro-4H-cyclopropa[e]pyrazolo[1,5-a]pyridine

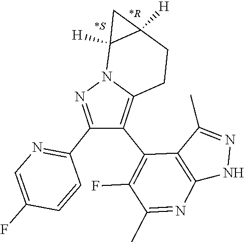 [SMILES: Cc1nc2[nH]nc(C)c2c(-c2c(-c3ccc(F)cn3)nn3c2CC[C@@H]2C[C@@H]23)c1F]

The title compound was prepared in a manner analogous to (5a*S,6a*R)-6,6-difluoro-3-(5-fluoro-1H-pyrazolo[3,4-b]pyridin-4-yl)-2-(5-fluoropyridin-2-yl)-5,5a,6,6a-tetrahydro-4H-cyclopropa[e]pyrazolo[1,5-a]pyridine (Example 358) except using (5a*R,6a*S)-3-bromo-2-(5-fluoropyridin-2-yl)-5,5a,6,6a-tetrahydro-4H-cyclopropa[e]pyrazolo[1,5-a]pyridine (Intermediate 179) instead of (5a*S,6a*R)-6,6-difluoro-3-bromo-2-(5-fluoropyridin-2-yl)-5,5a,6,6a-tetrahydro-4H-cyclopropa[e]pyrazolo[1,5-a]pyridine (Intermediate 182) and 5-fluoro-3,6-dimethyl-4-(tributylstannyl)-1-((2-(trimethylsilyl)ethoxy)methyl)-1H-pyrazolo[3,4-b]pyridine (Intermediate 236) instead of 5-fluoro-4-(tributylstannyl)-1-((2-(trimethylsilyl)ethoxy)methyl)-1H-pyrazolo[3,4-b]pyridine (Intermediate 235). MS (ESI): mass calcd. for C₂₁H₁₈F₂N₆ 392.2; m/z found 393.2 [M+H]⁺. ¹H NMR (400 MHz, CDCl₃): δ 8.09 (d, J=2.9 Hz, 1H), 7.81 (dd, J=8.8, 4.4 Hz, 1H), 7.38-7.30 (m, 1H), 4.04-3.92 (m, 1H), 2.75 (dd, J=3.4, 1.1 Hz, 3H), 2.72-2.12 (m, 4H), 2.03 (s, 3H), 1.80 (s, 1H), 1.34-1.16 (m, 1H), 1.12-0.90 (m, 1H).

### Example 347: (5a*R,6a*S)-2-(5-Fluoropyridin-2-yl)-3-(6-methyl-1H-pyrazolo[3,4-d]pyrimidin-4-yl)-5,5a,6,6a-tetrahydro-4H-cyclopropa[e]pyrazolo[1,5-a]pyridine

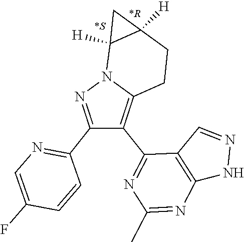 [SMILES: Cc1nc(-c2c(-c3ccc(F)cn3)nn3c2CC[C@@H]2C[C@@H]23)c2cn[nH]c2n1]

Step A. (5a*R,6a*S)-2-(5-Fluoropyridin-2-yl)-3-(4,4,5,5-tetramethyl-1,3,2-dioxaborolan-2-yl)-5,5a,6,6a-tetrahydro-4H-cyclopropa[e]pyrazolo[1,5-a]pyridine. (5a*R,6a*S)-3-Bromo-2-(5-fluoropyridin-2-yl)-5,5a,6,6a-tetrahydro-4H-cyclopropa[e]pyrazolo[1,5-a]pyridine (Intermediate 179, 500 mg, 1.62 mmol) was dissolved in dry THF (7 mL) under N₂. BPin isopropoxide (1.0 mL, 4.9 mml) was added and the reaction mixture was sparged for 5 minutes with N₂, cooled to −78° C., and treated with n-BuLi (1.6 M in hexanes, 3.0 mL, 4.9 mmol) then stirred at −78° C. for 1 hour. The mixture was warmed to r.t., quenched with sat. aq. NH₄Cl, extracted with EtOAc. The organic layer was washed with brine, concentrated, and purified on silica gel (0-100% EtOAc/hexanes) to yield the desired product (488 mg, 1.37 mmol, 85%). MS (ESI): mass calcd. for C₁₉H₂₃BFN₃O₂ 355.2; found 274.2 [M-Pin+H]⁺.

Step B. (5a*R,6a*S)-2-(5-Fluoropyridin-2-yl)-3-(6-methyl-1-((2-(trimethylsilyl)ethoxy)methyl)-1H-pyrazolo[3,4-d]pyrimidin-4-yl)-5,5a,6,6a-tetrahydro-4H-cyclopropa[e]pyrazolo[1,5-a]pyridine. A solution of (5a*R,6a*S)-2-(5-fluoropyridin-2-yl)-3-(4,4,5,5-tetramethyl-1,3,2-dioxaborolan-2-yl)-5,5a,6,6a-tetrahydro-4H-cyclopropa[e]pyrazolo[1,5-a]pyridine (120 mg, 0.34 mmol), 4-chloro-6-methyl-1-((2-(trimethylsilyl)ethoxy)methyl)-1H-pyrazolo[3,4-d]pyrimidine (92 mg, 0.31 mmol), XPhos Pd G4 (24 mg, 0.028 mmol) and Na₂CO₃ (2M aq., 0.44 mL, 0.87 mmol) were taken up in 1,4-dioxane (1.1 mL). The mixture was sparged with N₂ for 5 minutes, then heated to 50° C. for 2 hours. The reaction mixture was partitioned between ethyl acetate and water, the aqueous layer extracted with EtOAc, the combined organics washed with brine, concentrated, and purified on silica gel (0-100% EtOAc/hexanes) to obtain the desired product (135 mg, 0.275 mmol, 90%). MS (ESI): mass calcd. for C₂₅H₃₀FN₇OSi 491.2; found 492.3 [M+H]⁺.

Step C. (5a*R,6a*S)-2-(5-Fluoropyridin-2-yl)-3-(6-methyl-1H-pyrazolo[3,4-d]pyrimidin-4-yl)-5,5a,6,6a-tetrahydro-4H-cyclopropa[e]pyrazolo[1,5-a]pyridine. (5a*R,6a*S)-2-(5-Fluoropyridin-2-yl)-3-(6-methyl-1-((2-(trimethylsilyl)ethoxy)methyl)-1H-pyrazolo[3,4-d]pyrimidin-4-yl)-5,5a,6,6a-tetrahydro-4H-cyclopropa[e]pyrazolo[1,5-a]pyridine (136 mg, 0.276 mmol), HCl (6N aq., 2.7 mL, 16 mmol), ethanol (5.3 mL) and water (2.7 mL) were combined and heated in a sealed container at 80° C. for 1 hour. The reaction mixture was neutralized with aq. Na₂CO₃ and extracted with DCM. The organic layer was concentrated and the residue purified by reverse-phase HPLC (AccuPrep, 10-100% MeCN/water, NH₄OH modifier) to obtain the title compound (20 mg, 0.055 mmol, 20% yield). MS (ESI): mass calcd. for C₁₉H₁₆FN₇ 361.1; found, 362.2 [M+H]⁺. ¹H NMR (400 MHz, CDCl₃): δ 11.73-11.59 (m, 1H), 8.29-8.20 (m, 1H), 7.75-7.63 (m, 1H), 7.42-7.31 (m, 1H), 7.20-7.12 (m, 1H), 3.99-3.90 (m, 1H), 3.26-3.13 (m, 1H), 2.90-2.82 (m, 3H), 2.77-2.64 (m, 1H), 2.20-2.02 (m, 2H), 1.82-1.73 (m, 1H), 1.22-1.12 (m, 1H), 1.08-0.98 (m, 1H).

### Example 348: (Racemic) N-(4-(6,6-Difluoro-2-(4-fluorophenyl)-5,5a,6,6a-tetrahydro-4H-cyclopropa[e]pyrazolo[1,5-a]pyridin-3-yl)pyridin-2-yl)acetamide

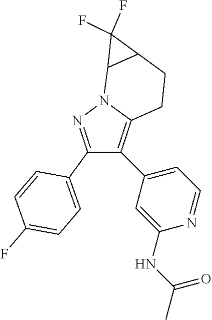 [SMILES: CC(=O)Nc1cc(-c2c(-c3ccc(F)cc3)nn3c2CCC2C3C2(F)F)ccn1]

The title compound was prepared in a manner analogous to (5a*R,6a*S)-6,6-difluoro-2-(4-fluorophenyl)-3-(pyrazolo[1,5-a]pyridin-4-yl)-5,5a,6,6a-tetrahydro-4H-cyclopropa[e]pyrazolo[1,5-a]pyridine (Example 354), except that N-(4-(4,4,5,5-tetramethyl-1,3,2-dioxaborolan-2-yl)pyridin-2-yl)acetamide was used instead of pyrazolo[1,5-a]pyridin-5-ylboronic acid and no chiral SFC was performed. MS (ESI): mass calcd. for C₂₁H₁₇F₃N₄O 398.1; m/z found 399.1 [M+H]⁺. ¹H NMR (500 MHz, CDCl₃): δ 8.14-8.09 (m, 2H), 7.89 (s, 1H), 7.45-7.36 (m, 2H), 7.05-6.95 (m, 2H), 6.71 (dd, J=5.2, 1.6 Hz, 1H), 4.25 (dd, J=10.8, 6.3 Hz, 1H), 2.98-2.79 (m, 2H), 2.49-2.34 (m, 1H), 2.21 (s, 5H).

### Example 349: N-(4-((5a*R,6a*S-6,6-Difluoro-2-(4-fluorophenyl)-5,5a,6,6a-tetrahydro-4H-cyclopropa[e]pyrazolo[1,5-a]pyridin-3-yl)pyridin-2-yl)acetamide

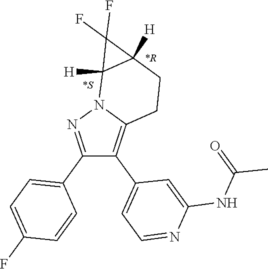 [SMILES: CC(=O)Nc1cc(-c2c(-c3ccc(F)cc3)nn3c2CC[C@@H]2[C@H]3C2(F)F)ccn1]

The title compound was prepared in a manner analogous to (5a*R,6a*S)-6,6-difluoro-2-(4-fluorophenyl)-3-(pyrazolo[1,5-a]pyridin-4-yl)-5,5a,6,6a-tetrahydro-4H-cyclopropa[e]pyrazolo[1,5-a]pyridine (Example 354), N-(4-(4,4,5,5-tetramethyl-1,3,2-dioxaborolan-2-yl)pyridin-2-yl)acetamide was used instead of pyrazolo[1,5-a]pyridin-5-ylboronic acid, and a modified chiral SFC method was used (Stationary phase: Whelk O1 SS Sum 250×21 mm, Mobile phase: 25% methanol, 75% CO₂). MS (ESI): mass calcd. for C₂₁H₁₇F₃N₄O 398.1; m/z found 399.2 [M+H]⁺. ¹H NMR (400 MHz, CDCl₃): δ 8.58 (s, 1H), 8.14 (d, J=4.6 Hz, 2H), 7.48-7.39 (m, 2H), 7.02 (t, J=8.7 Hz, 2H), 6.74 (dd, J=5.2, 1.5 Hz, 1H), 4.28 (dd, J=10.8, 6.2 Hz, 1H), 3.03-2.79 (m, 2H), 2.51-2.37 (m, 1H), 2.30-2.03 (m, 5H).

### Example 350: N-(4-((5a*S,6a*R)-6,6-Difluoro-2-(4-fluorophenyl)-5,5a,6,6a-tetrahydro-4H-cyclopropa[e]pyrazolo[1,5-a]pyridin-3-yl)pyridin-2-yl)acetamide

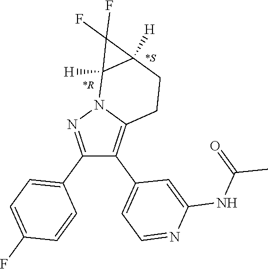 [SMILES: CC(=O)Nc1cc(-c2c(-c3ccc(F)cc3)nn3c2CC[C@H]2[C@@H]3C2(F)F)ccn1]

The title compound was prepared in a manner analogous to (5a*R,6a*S)-6,6-difluoro-2-(4-fluorophenyl)-3-(pyrazolo[1,5-a]pyridin-4-yl)-5,5a,6,6a-tetrahydro-4H-cyclopropa[e]pyrazolo[1,5-a]pyridine (Example 354), N-(4-(4,4,5,5-tetramethyl-1,3,2-dioxaborolan-2-yl)pyridin-2-yl)acetamide was used instead of pyrazolo[1,5-a]pyridin-5-ylboronic acid, and a modified chiral SFC method was used (Stationary phase: Whelk O1 SS Sum 250×21 mm, Mobile phase: 25% methanol, 75% CO₂). MS (ESI): mass calcd. for C₂₁H₁₇F₃N₄O 398.1; m/z found 399.2 [M+H]⁺. ¹H NMR (400 MHz, CDCl₃): δ 8.62 (s, 1H), 8.14 (d, J=4.7 Hz, 2H), 7.44 (dd, J=8.7, 5.5 Hz, 2H), 7.09-6.96 (m, 2H), 6.74 (dd, J=5.2, 1.6 Hz, 1H), 4.28 (dd, J=10.8, 6.3 Hz, 1H), 3.05-2.77 (m, 2H), 2.54-2.38 (m, 1H), 2.27-2.06 (m, 5H).

### Example 351: N-(4-((5a*S,6a*R)-6,6-Difluoro-2-(5-fluoropyridin-2-yl)-5,5a,6,6a-tetrahydro-4H-cyclopropa[e]pyrazolo[1,5-a]pyridin-3-yl)pyridin-2-yl)acetamide

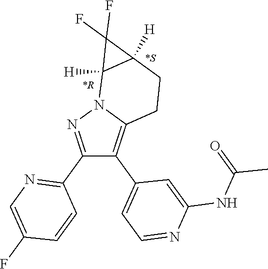 [SMILES: CC(=O)Nc1cc(-c2c(-c3ccc(F)cn3)nn3c2CC[C@H]2[C@@H]3C2(F)F)ccn1]

The title compound was prepared in a manner analogous to (5a*R,6a*S)-6,6-difluoro-2-(4-fluorophenyl)-3-(pyrazolo[1,5-a]pyridin-4-yl)-5,5a,6,6a-tetrahydro-4H-cyclopropa[e]pyrazolo[1,5-a]pyridine (Example 354), except that (5a*S,6a*R)-6,6-difluoro-3-bromo-2-(5-fluoropyridin-2-yl)-5,5a,6,6a-tetrahydro-4H-cyclopropa[e]pyrazolo[1,5-a]pyridine (Intermediate 182) was used instead of racemic 3-bromo-6,6-difluoro-2-(4-fluorophenyl)-5,5a,6,6a-tetrahydro-4H-cyclopropa[e]pyrazolo[1,5-a]pyridine (Intermediate 181), N-(4-(4,4,5,5-tetramethyl-1,3,2-dioxaborolan-2-yl)pyridin-2-yl)acetamide was used instead of pyrazolo[1,5-a]pyridin-5-ylboronic acid, and no chiral separation was performed. MS (ESI): mass calcd. for C₂₀H₁₆F₃N₅O 399.1; m/z found 400.2 [M+H]⁺. ¹H NMR (400 MHz, CDCl₃): δ 8.45-8.33 (m, 1H), 8.15 (dd, J=5.2, 0.8 Hz, 1H), 8.10 (s, 1H), 7.93 (s, 1H), 7.64-7.57 (m, 1H), 7.43-7.34 (m, 1H), 6.84 (dd, J=5.2, 1.6 Hz, 1H), 4.30 (dd, J=10.9, 6.3 Hz, 1H), 3.02-2.76 (m, 2H), 2.48-2.35 (m, 1H), 2.26-2.05 (m, 5H).

### Example 352: N-(4-((5a*R,6a*S)-6,6-Difluoro-2-(5-fluoropyridin-2-yl)-5,5a,6,6a-tetrahydro-4H-cyclopropa[e]pyrazolo[1,5-a]pyridin-3-yl)pyridin-2-yl)acetamide

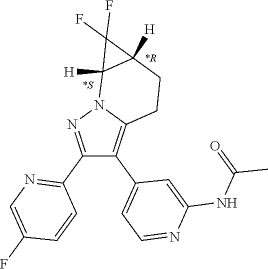 [SMILES: CC(=O)Nc1cc(-c2c(-c3ccc(F)cn3)nn3c2CC[C@@H]2[C@H]3C2(F)F)ccn1]

The title compound was prepared in a manner analogous to (5a*R,6a*S)-6,6-difluoro-2-(4-fluorophenyl)-3-(pyrazolo[1,5-a]pyridin-4-yl)-5,5a,6,6a-tetrahydro-4H-cyclopropa[e]pyrazolo[1,5-a]pyridine (Example 354), except that (5a*R,6a*S)-6,6-difluoro-3-bromo-2-(5-fluoropyridin-2-yl)-5,5a,6,6a-tetrahydro-4H-cyclopropa[e]pyrazolo[1,5-a]pyridine (Intermediate 183) was used instead of racemic 3-bromo-6,6-difluoro-2-(4-fluorophenyl)-5,5a,6,6a-tetrahydro-4H-cyclopropa[e]pyrazolo[1,5-a]pyridine (Intermediate 181), N-(4-(4,4,5,5-tetramethyl-1,3,2-dioxaborolan-2-yl)pyridin-2-yl)acetamide was used instead of pyrazolo[1,5-a]pyridin-5-ylboronic acid, and no chiral separation was performed. MS (ESI): mass calcd. for C₂₀H₁₆F₃N₅O 399.1; m/z found 400.2 [M+H]⁺. ¹H NMR (400 MHz, CDCl₃): δ 8.47-8.36 (m, 1H), 8.15 (dd, J=5.2, 0.8 Hz, 1H), 8.10 (s, 1H), 7.95 (s, 1H), 7.68-7.54 (m, 1H), 7.48-7.35 (m, 1H), 6.84 (dd, J=5.2, 1.5 Hz, 1H), 4.39-4.22 (m, 1H), 3.06-2.77 (m, 2H), 2.51-2.38 (m, 1H), 2.26-2.09 (m, 5H).

### Example 353: (5a*S,6a*R)-6,6-Difluoro-2-(4-fluorophenyl)-3-(pyrazolo[1,5-a]pyridin-4-yl)-5,5a,6,6a-tetrahydro-4H-cyclopropa[e]pyrazolo[1,5-a]pyridine

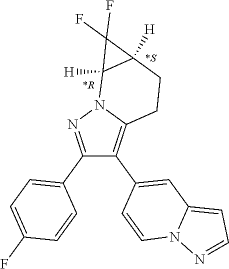 [SMILES: Fc1ccc(-c2nn3c(c2-c2ccn4nccc4c2)CC[C@H]2[C@@H]3C2(F)F)cc1]

Pyrazolo[1,5-a]pyridin-5-ylboronic acid (25 mg, 0.15 mmol), racemic 3-bromo-6,6-difluoro-2-(4-fluorophenyl)-5,5a,6,6a-tetrahydro-4H-cyclopropa[e]pyrazolo[1,5-a]pyridine (Intermediate 181, 40 mg, 0.12 mmol), CatacXium Pd G4 (8.7 mg, 0.012 mmol), and Cs₂CO₃ (114 mg, 0.35 mmol) were dissolved in a 2:1 mixture of t-amyl alcohol and water (3 mL). The biphasic mixture was stirred vigorously for 2 hours at 90° C., cooled to room temperature, partitioned between water and DCM, and the aqueous layer extracted 2×DCM. The combined organics were concentrated and purified on silica gel (0-100% EtOAc/hexanes) and the resulting racemic material was resolved using chiral SFC (Stationary phase: Whelk O1 SS Sum 250×21 mm, Mobile phase: 20% methanol, 80% CO₂) to yield 13.1 mg (0.34 mmol, 30%) of the title compound. MS (ESI): mass calcd. for C₂₁H₁₅F₃N₄ 380.1; m/z found 381.2 [M+H]⁺. ¹H NMR (600 MHz, CDCl₃): δ 8.41-8.33 (m, 1H), 7.96 (d, J=2.3 Hz, 1H), 7.50-7.44 (m, 2H), 7.34 (dd, J=1.9, 1.0 Hz, 1H), 7.03-6.97 (m, 2H), 6.52-6.39 (m, 2H), 4.28 (dd, J=10.8, 6.3 Hz, 1H), 2.92-2.73 (m, 2H), 2.48-2.37 (m, 1H), 2.26-2.02 (m, 2H).

### Example 354: (5a*R,6a*S)-6,6-Difluoro-2-(4-fluorophenyl)-3-(pyrazolo[1,5-a]pyridin-4-yl)-5,5a,6,6a-tetrahydro-4H-cyclopropa[e]pyrazolo[1,5-a]pyridine

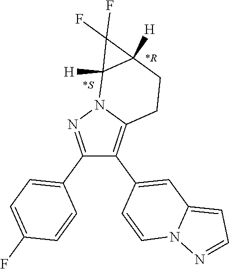 [SMILES: Fc1ccc(-c2nn3c(c2-c2ccn4nccc4c2)CC[C@@H]2[C@H]3C2(F)F)cc1]

Pyrazolo[1,5-a]pyridin-5-ylboronic acid (25 mg, 0.15 mmol), racemic 3-bromo-6,6-difluoro-2-(4-fluorophenyl)-5,5a,6,6a-tetrahydro-4H-cyclopropa[e]pyrazolo[1,5-a]pyridine (Intermediate 181, 40 mg, 0.12 mmol), CatacXium Pd G4 (8.7 mg, 0.012 mmol), and Cs₂CO₃ (114 mg, 0.35 mmol) were dissolved in a 2:1 mixture of t-amyl alcohol and water (3 mL). The biphasic mixture was stirred vigorously for 2 hours at 90° C., cooled to room temperature, partitioned between water and DCM, and the aqueous layer extracted 2×DCM. The combined organics were concentrated and purified on silica gel (0-100% EtOAc/hexanes) and the resulting racemic material was resolved using chiral SFC (Stationary phase: Whelk O1 SS Sum 250×21 mm, Mobile phase: 20% methanol, 80% CO₂) to yield 13 mg (0.34 mmol, 29%) of the title compound. MS (ESI): mass calcd. for C₂₁H₁₅F₃N₄ 380.1; m/z found 381.1 [M+H]⁺. ¹H NMR (600 MHz, CDCl₃): δ 8.43-8.35 (m, 1H), 7.96 (d, J=2.3 Hz, 1H), 7.50-7.40 (m, 2H), 7.34 (dd, J=1.9, 1.0 Hz, 1H), 7.04-6.96 (m, 2H), 6.50-6.44 (m, 2H), 4.28 (dd, J=10.8, 6.3 Hz, 1H), 2.93-2.73 (m, 2H), 2.43 (s, 1H), 2. 26-2.07 (m, 2H).

### Example 355: (5a*S,6a*R)-6,6-Difluoro-2-(4-fluorophenyl)-3-(1H-pyrazolo[3,4-b]pyridin-4-yl)-5,5a,6,6a-tetrahydro-4H-cyclopropa[e]pyrazolo[1,5-a]pyridine

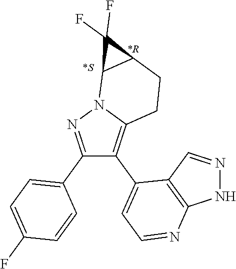 [SMILES: Fc1ccc(-c2nn3c(c2-c2ccnc4[nH]ncc24)CC[C@H]2[C@@H]3C2(F)F)cc1]

The title compound was prepared in a manner analogous to 4-(6,6-difluoro-2-(4-fluorophenyl)-4,5,6,7-tetrahydropyrazolo[1,5-a]pyridin-3-yl)-5-fluoro-1H-pyrazolo[3,4-b]pyridine (Example 280), except using 4-(4,4,5,5-tetramethyl-1,3,2-dioxaborolan-2-yl)-1-((2-(trimethylsilyl)ethoxy)methyl)-1H-pyrazolo[3,4-b]pyridine (Intermediate 23) instead of 5-Fluoro-4-(4,4,5,5-tetramethyl-1,3,2-dioxaborolan-2-yl)-1-((2-(trimethylsilyl)ethoxy)methyl)-1H-pyrazolo[3,4-b]pyridine (Intermediate 222) and racemic 3-bromo-6,6-difluoro-2-(4-fluorophenyl)-5,5a,6,6a-tetrahydro-4H-cyclopropa[e]pyrazolo[1,5-a]pyridine (Intermediate 181) instead of 3-bromo-6,6-difluoro-2-(4-fluorophenyl)-4,5,6,7-tetrahydropyrazolo[1,5-a]pyridine (Intermediate 52). The title compound was separated from its enantiomer using chiral SFC (Stationary phase: Chiralcel OZ3 Sum 250×21 mm, Mobile phase: 20% methanol, 80% CO₂). MS (ESI): mass calcd. for C₂₀H₁₄F₃N₅ 381.1; m/z found 382.1 [M+H]⁺. ¹H NMR (600 MHz, CDCl₃): δ 12.77 (s, 1H), 8.60 (d, J=4.8 Hz, 1H), 7.58 (s, 1H), 7.42-7.32 (m, 2H), 6.97 (d, J=4.8 Hz, 1H), 6.95-6.86 (m, 2H), 4.34 (dd, J=10.9, 6.1 Hz, 1H), 2.93-2.68 (m, 2H), 2.53-2.39 (m, 1H), 2.30-2.06 (m, 2H).

### Example 356: (5a*R,6a*S-6,6-Difluoro-2-(4-fluorophenyl)-3-(1H-pyrazolo[3,4-b]pyridin-4-yl)-5,5a,6,6a-tetrahydro-4H-cyclopropa[e]pyrazolo[1,5-a]pyridine

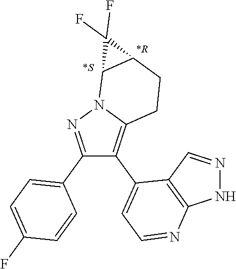 [SMILES: Fc1ccc(-c2nn3c(c2-c2ccnc4[nH]ncc24)CC[C@@H]2[C@H]3C2(F)F)cc1]

The title compound was prepared in a manner analogous to 4-(6,6-difluoro-2-(4-fluorophenyl)-4,5,6,7-tetrahydropyrazolo[1,5-a]pyridin-3-yl)-5-fluoro-1H-pyrazolo[3,4-b]pyridine (Example 280), except using 4-(4,4,5,5-tetramethyl-1,3,2-dioxaborolan-2-yl)-1-((2-(trimethylsilyl)ethoxy)methyl)-1H-pyrazolo[3,4-b]pyridine (Intermediate 23) instead of 5-fluoro-4-(4,4,5,5-tetramethyl-1,3,2-dioxaborolan-2-yl)-1-((2-(trimethylsilyl)ethoxy)methyl)-1H-pyrazolo[3,4-b]pyridine (Intermediate 222) and racemic 3-bromo-6,6-difluoro-2-(4-fluorophenyl)-5,5a,6,6a-tetrahydro-4H-cyclopropa[e]pyrazolo[1,5-a]pyridine (Intermediate 181) instead of 3-bromo-6,6-difluoro-2-(4-fluorophenyl)-4,5,6,7-tetrahydropyrazolo[1,5-a]pyridine (Intermediate 52). The title compound was separated from its enantiomer using chiral SFC (Stationary phase: Chiralcel OZ3 Sum 250×21 mm, Mobile phase: 20% methanol, 80% CO₂). MS (ESI): mass calcd. for C₂₀H₁₄F₃N₅ 381.1; m/z found 382.1 [M+H]⁺. ¹H NMR (600 MHz, CDCl₃): δ 12.67 (s, 1H), 8.59 (d, J=4.8 Hz, 1H), 7.58 (s, 1H), 7.41-7.35 (m, 2H), 6.97 (d, J=4.8 Hz, 1H), 6.95-6.89 (m, 2H), 4.34 (dd, J=10.9, 6.1 Hz, 1H), 2.90-2.72 (m, 2H), 2.53-2.41 (m, 1H), 2.26-2.07 (m, 2H).

### Example 357: (5a*S,6a*R)-6,6-Difluoro-3-(5-fluoro-6-methyl-1H-pyrazolo[3,4-b]pyridin-4-yl)-2-(5-fluoropyridin-2-yl)-5,5a,6,6a-tetrahydro-4H-cyclopropa[e]pyrazolo[1,5-a]pyridine

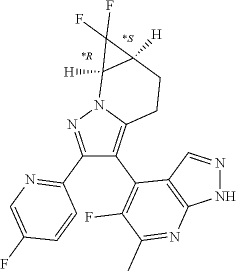 [SMILES: Cc1nc2[nH]ncc2c(-c2c(-c3ccc(F)cn3)nn3c2CC[C@H]2[C@@H]3C2(F)F)c1F]

The title compound was prepared in a manner analogous to (5a*S,6a*R)-6,6-difluoro-3-(5-fluoro-1H-pyrazolo[3,4-b]pyridin-4-yl)-2-(5-fluoropyridin-2-yl)-5,5a,6,6a-tetrahydro-4H-cyclopropa[e]pyrazolo[1,5-a]pyridine (Example 358) except 5-fluoro-6-methyl-1-(tetrahydro-2H-pyran-2-yl)-4-(tributylstannyl)-1H-pyrazolo[3,4-b]pyridine (Intermediate 237) instead of 5-fluoro-4-(tributylstannyl)-1-((2-(trimethylsilyl)ethoxy)methyl)-1H-pyrazolo[3,4-b]pyridine (Intermediate 235) and 4N HCl in dioxane instead of TFA. MS (ESI): mass calcd. for C₂₀H₁₄F₄N₆ 414.1; m/z found 415.2 [M+H]⁺. ¹H NMR (400 MHz, CDCl₃): δ 8.11 (dd, J=8.4, 2.9 Hz, 1H), 7.96-7.78 (m, 1H), 7.52 (d, J=16.5 Hz, 1H), 7.47-7.39 (m, 1H), 4.47-4.20 (m, 1H), 2.91-2.59 (m, 5H), 2.56-2.40 (m, 1H), 2.36-2.03 (m, 2H). N—H proton not observed.

### Example 358: (5a*S,6a*R)-6,6-Difluoro-3-(5-fluoro-1H-pyrazolo[3,4-b]pyridin-4-yl)-2-(5-fluoropyridin-2-yl)-5,5a,6,6a-tetrahydro-4H-cyclopropa[e]pyrazolo[1,5-a]pyridine

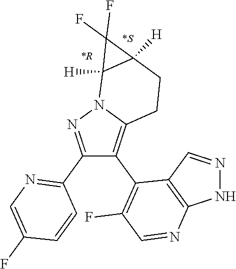 [SMILES: Fc1ccc(-c2nn3c(c2-c2c(F)cnc4[nH]ncc24)CC[C@H]2[C@@H]3C2(F)F)nc1]

Step A: (5a*S,6a*R)-6,6-Difluoro-3-(5-fluoro-1-((2-(trimethylsilyl)ethoxy)methyl)-1H-pyrazolo[3,4-b]pyridin-4-yl)-2-(5-fluoropyridin-2-yl)-5,5a,6,6a-tetrahydro-4H-cyclopropa[e]pyrazolo[1,5-a]pyridine. To a mixture of (5a*S,6a*R)-6,6-difluoro-3-bromo-2-(5-fluoropyridin-2-yl)-5,5a,6,6a-tetrahydro-4H-cyclopropa[e]pyrazolo[1,5-a]pyridine (Intermediate 182, 40 mg, 0.12 mmol), 5-fluoro-4-(tributylstannyl)-1-((2-(trimethylsilyl)ethoxy)methyl)-1H-pyrazolo[3,4-b]pyridine (Intermediate 235, 78 mg, 0.14 mmol), and PdCl₂(PPh₃)₂ (8.2 mg, 0.012 mmol) in dioxane (3 mL) was added copper(I) iodide (4.4 mg, 0.023 mmol). The reaction vessel was sealed, the mixture was sparged with N₂ for 5 minutes, and then heated to 150° C. in a microwave reactor for 2 hours. The mixture was filtered, concentrated, and purified on silica gel (0-70% EA/hexanes) to obtain 34 mg (0.064 mmol, 55%) of the desired product. MS (ESI): mass calcd. for C₂₅H₁₆F₄N₆OSi 530.2; m/z found 531.2 [M+H]⁺.

Step B: (5a*S,6a*R)-6,6-difluoro-3-(5-fluoro-1H-pyrazolo[3,4-b]pyridin-4-yl)-2-(5-fluoropyridin-2-yl)-5,5a,6,6a-tetrahydro-4H-cyclopropa[e]pyrazolo[1,5-a]pyridine. (5a*S,6a*R)-6,6-Difluoro-3-(5-fluoro-1-((2-(trimethylsilyl)ethoxy)methyl)-1H-pyrazolo[3,4-b]pyridin-4-yl)-2-(5-fluoropyridin-2-yl)-5,5a,6,6a-tetrahydro-4H-cyclopropa[e]pyrazolo[1,5-a]pyridine (34 mg, 0.064 mmol) was dissolved in TFA and the mixture was stirred at r.t. for 4 hours, then concentrated. The residue was purified by reverse-phase HPLC (AccuPrep, 0-100% MeCN/water, NH₄OH modifier) to obtain (7.4 mg, 0.019 mmol, 29%) of the title compound. MS (ESI): mass calcd. for C₁₉H₁₂F₄N₆ 400.1; m/z found 401.1 [M+H]⁺. ¹H NMR (400 MHz, CDCl₃): δ 10.56 (s, 1H), 8.47 (d, J=2.6 Hz, 1H), 8.15 (dd, J=11.3, 2.9 Hz, 1H), 7.86-7.71 (m, 1H), 7.52 (d, J=7.1 Hz, 1H), 7.41-7.32 (m, 1H), 4.44-4.26 (m, 1H), 2.89-2.54 (m, 2H), 2.55-2.38 (m, 1H), 2.32-1.99 (m, 2H).

### Example 359: (5a*S,6a*R)-6,6-Difluoro-3-(5-fluoro-3-methyl-1H-pyrazolo[3,4-b]pyridin-4-yl)-2-(5-fluoropyridin-2-yl)-5,5a,6,6a-tetrahydro-4H-cyclopropa[e]pyrazolo[1,5-a]pyridine

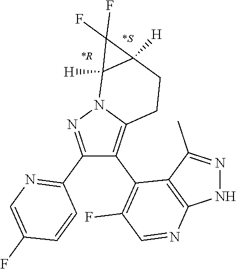 [SMILES: Cc1n[nH]c2ncc(F)c(-c3c(-c4ccc(F)cn4)nn4c3CC[C@H]3[C@@H]4C3(F)F)c12]

The title compound was prepared in a manner analogous to (5a*S,6a*R)-6,6-difluoro-3-(5-fluoro-1H-pyrazolo[3,4-b]pyridin-4-yl)-2-(5-fluoropyridin-2-yl)-5,5a,6,6a-tetrahydro-4H-cyclopropa[e]pyrazolo[1,5-a]pyridine (Example 358) except 5-fluoro-3-methyl-1-(tetrahydro-2H-pyran-2-yl)-4-(tributylstannyl)-1H-pyrazolo[3,4-b]pyridine (Intermediate 238) instead of 5-fluoro-4-(tributylstannyl)-1-((2-(trimethylsilyl)ethoxy)methyl)-1H-pyrazolo[3,4-b]pyridine (Intermediate 235) and 4N HCl in dioxane instead of TFA. MS (ESI): mass calcd. for C₂₀H₁₄F₄N₆ 414.1; m/z found 415.1 [M+H]⁺. ¹H NMR (400 MHz, CDCl₃): δ 8.54 (d, J=2.2 Hz, 1H), 8.09 (d, J=2.9 Hz, 1H), 7.90-7.77 (m, 1H), 7.41-7.32 (m, 1H), 4.40 (dd, J=10.8, 6.1 Hz, 1H), 2.80-2.66 (m, 1H), 2.59-2.41 (m, 2H), 2.32-2.20 (m, 1H), 2.13 (dt, J=13.1, 6. 6 Hz, 1H), 2.04 (s, 3H). N—H proton not observed

### Example 360: (5a*S,6a*R)-6,6-Difluoro-3-(5-fluoro-3,6-dimethyl-1H-pyrazolo[3,4-b]pyridin-4-yl)-2-(5-fluoropyridin-2-yl)-5,5a,6,6a-tetrahydro-4H-cyclopropa[e]pyrazolo[1,5-a]pyridine

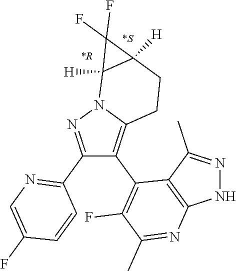 [SMILES: Cc1nc2[nH]nc(C)c2c(-c2c(-c3ccc(F)cn3)nn3c2CC[C@H]2[C@@H]3C2(F)F)c1F]

The title compound was prepared in a manner analogous to (5a*S,6a*R)-6,6-difluoro-3-(5-fluoro-1H-pyrazolo[3,4-b]pyridin-4-yl)-2-(5-fluoropyridin-2-yl)-5,5a,6,6a-tetrahydro-4H-cyclopropa[e]pyrazolo[1,5-a]pyridine (Example 358) except 5-fluoro-3,6-dimethyl-4-(tributylstannyl)-1-((2-(trimethylsilyl)ethoxy)methyl)-1H-pyrazolo[3,4-b]pyridine (Intermediate 236) instead of 5-fluoro-4-(tributylstannyl)-1-((2-(trimethylsilyl)ethoxy)methyl)-1H-pyrazolo[3,4-b]pyridine (Intermediate 235). MS (ESI): mass calcd. for C₂₁H₁₆F₄N₆ 428.1; found, 429.3 [M+H]⁺. ¹H NMR (500 MHz, CDCl₃): δ 8.03 (d, J=2.8 Hz, 1H), 8.01-7.95 (m, 1H), 7.47-7.39 (m, 1H), 4.42 (dd, J=10.8, 6.1 Hz, 1H), 2.80 (d, J=3.1 Hz, 3H), 2.68-2.62 (m, 2H), 2.57-2.47 (m, 1H), 2.26-2.15 (m, 2H), 2.07 (s, 3H). N—H proton not observed.

### Example 361: (5a*S,6a*R)-6,6-Difluoro-2-(5-fluoropyridin-2-yl)-3-(6-methyl-1H-pyrazolo[3,4-d]pyrimidin-4-yl)-5,5a,6,6a-tetrahydro-4H-cyclopropa[e]pyrazolo[1,5-a]pyridine

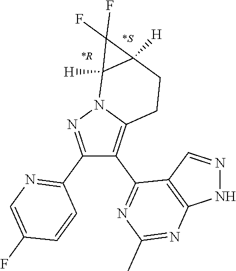 [SMILES: Cc1nc(-c2c(-c3ccc(F)cn3)nn3c2CC[C@H]2[C@@H]3C2(F)F)c2cn[nH]c2n1]

The title compound was prepared in a manner analogous to (5a*R,6a*S)-2-(5-fluoropyridin-2-yl)-3-(6-methyl-1H-pyrazolo[3,4-d]pyrimidin-4-yl)-5, 5a,6,6a-tetrahydro-4H-cyclopropa[e]pyrazolo[1,5-a]pyridine (Example 347), except using (5a*S,6a*R)-6,6-difluoro-3-bromo-2-(5-fluoropyridin-2-yl)-5,5a,6,6a-tetrahydro-4H-cyclopropa[e]pyrazolo[1,5-a]pyridine (Intermediate 182) instead of (5a*R,6a*S)-3-bromo-2-(5-fluoropyridin-2-yl)-5,5a,6,6a-tetrahydro-4H-cyclopropa[e]pyrazolo[1,5-a]pyridine (Intermediate 179). MS (ESI): mass calcd. for C₁₉H₁₄F₃N₇ 397.1; found 398.2 [M+H]⁺. ¹H NMR (400 MHz, CDCl₃): δ 11.77 (br s, 1H), 8.23 (d, J=2.88 Hz, 1H), 7.76 (dd, J=8.76, 4.38 Hz, 1H), 7.40 (td, J=8.41, 2.94 Hz, 1H), 7.17 (s, 1H), 4.35 (dd, J=10.82, 6.32 Hz, 1H), 3.21-3.12 (m, 1H), 3.10-2.99 (m, 1H), 2.87 (s, 3H), 2.54-2.42 (m, 1H), 2.26-2.12 (m, 2H).

### Example 362: (4a*R,5a*R)-5,5-Difluoro-2-(4-fluorophenyl)-3-(1H-pyrazolo[3,4-b]pyridin-4-yl)-4a,5,5a,6-tetrahydro-4H-cyclopropa[d]pyrazolo[1,5-a]pyridine

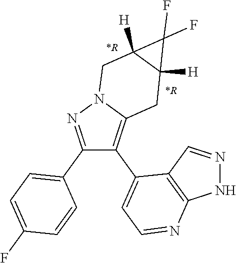 [SMILES: Fc1ccc(-c2nn3c(c2-c2ccnc4[nH]ncc24)C[C@@H]2[C@H](C3)C2(F)F)cc1]

The title compound was prepared in a manner analogous to 4-(6,6-difluoro-2-(4-fluorophenyl)-4,5,6,7-tetrahydropyrazolo[1,5-a]pyridin-3-yl)-5-fluoro-1H-pyrazolo[3,4-b]pyridine (Example 280), except using 4-(4,4,5,5-tetramethyl-1,3,2-dioxaborolan-2-yl)-1-((2-(trimethylsilyl)ethoxy)methyl)-1H-pyrazolo[3,4-b]pyridine (Intermediate 23) instead of 5-fluoro-4-(4,4,5,5-tetramethyl-1,3,2-dioxaborolan-2-yl)-1-((2-(trimethylsilyl)ethoxy)methyl)-1H-pyrazolo[3,4-b]pyridine (Intermediate 222), racemic 3-bromo-5,5-difluoro-2-(4-fluorophenyl)-4a,5,5a,6-tetrahydro-4H-cyclopropa[d]pyrazolo[1,5-a]pyridine (Intermediate 177) instead of 3-bromo-6,6-difluoro-2-(4-fluorophenyl)-4,5,6,7-tetrahydropyrazolo[1,5-a]pyridine (Intermediate 52), and a modified SFC method (Stationary phase: Chiralpak IB N₃ Sum 250×21 mm, Mobile phase: 20% methanol with 0.2% triethylamine, 80% CO₂). MS (ESI): mass calcd. for C₂₀H₁₄F₃N₅ 381.1; m/z found 382.2 [M+H]⁺. ¹H NMR (600 MHz, CDCl₃): δ 12.35 (s, 1H), 8.59 (d, J=4.7 Hz, 1H), 7.59 (s, 1H), 7.36-7.30 (m, 2H), 6.99 (d, J=4.8 Hz, 1H), 6.93-6.87 (m, 2H), 4.75-4.61 (m, 1H), 4.57-4.44 (m, 1H), 3.28-3.21 (m, 1H), 3.08 (dd, J=17.5, 2.4 Hz, 1H), 2.40-2.07 (m, 2H).

### Example 363: (4a*S,5a*S)-5,5-Difluoro-2-(4-fluorophenyl)-3-(1H-pyrazolo[3,4-b]pyridin-4-yl)-4a,5,5a,6-tetrahydro-4H-cyclopropa[d]pyrazolo[1,5-a]pyridine

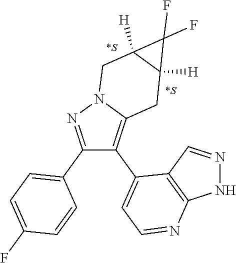 [SMILES: Fc1ccc(-c2nn3c(c2-c2ccnc4[nH]ncc24)C[C@H]2[C@@H](C3)C2(F)F)cc1]

The title compound was prepared in a manner analogous to 4-(6,6-Difluoro-2-(4-fluorophenyl)-4,5,6,7-tetrahydropyrazolo[1,5-a]pyridin-3-yl)-5-fluoro-1H-pyrazolo[3,4-b]pyridine (Example 280), except using 4-(4,4,5,5-tetramethyl-1,3,2-dioxaborolan-2-yl)-1-((2-(trimethylsilyl)ethoxy)methyl)-1H-pyrazolo[3,4-b]pyridine (Intermediate 23) instead of 5-fluoro-4-(4,4,5,5-tetramethyl-1,3,2-dioxaborolan-2-yl)-1-((2-(trimethylsilyl)ethoxy)methyl)-1H-pyrazolo[3,4-b]pyridine (Intermediate 222), racemic 3-bromo-5,5-difluoro-2-(4-fluorophenyl)-4a,5,5a,6-tetrahydro-4H-cyclopropa[d]pyrazolo[1,5-a]pyridine (Intermediate 177) instead of 3-bromo-6,6-difluoro-2-(4-fluorophenyl)-4,5,6,7-tetrahydropyrazolo[1,5-a]pyridine (Intermediate 52) and a modified SFC method (Stationary phase: Chiralpak IB N₃ Sum 250×21 mm, Mobile phase: 20% methanol with 0.2% triethylamine, 80% CO₂). MS (ESI): mass calcd. for C₂₀H₁₄F₃N₅ 381.1; m/z found 382.2 [M+H]⁺. ¹H NMR (600 MHz, CDCl₃): δ 12.31 (s, 1H), 8.59 (d, J=4.8 Hz, 1H), 7.59 (s, 1H), 7.37-7.31 (m, 2H), 6.99 (d, J=4.8 Hz, 1H), 6.93-6.86 (m, 2H), 4.68-4.61 (m, 1H), 4.56-4.49 (m, 1H), 3.27-3.20 (m, 1H), 3.08 (dd, J=17.3, 2.4 Hz, 1H), 2.37-2.07 (m, 2H).

### Example 364: (4*R,7*S)-2-(4-Fluorophenyl)-3-(6-methyl-1H-pyrazolo[3,4-b]pyridin-4-yl)-4,5,6,7-tetrahydro-4,7-methanopyrazolo[1,5-a]pyridine

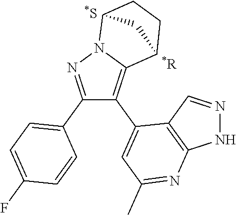 [SMILES: Cc1cc(-c2c(-c3ccc(F)cc3)nn3c2[C@@H]2CC[C@H]3C2)c2cn[nH]c2n1]

The title compound was prepared in a manner analogous to Example 1, Steps A-B, except using 3-bromo-2-(4-fluorophenyl)-4,5,6,7-tetrahydro-4,7-methanopyrazolo[1,5-a]pyridine (Intermediate 143) instead of 3-bromo-2-(5-fluoropyridin-2-yl)-6,7-dihydro-4H-pyrazolo[5,1-c][1,4]oxazine (Intermediate 37); and 6-methyl-4-(4,4,5,5-tetramethyl-1,3,2-dioxaborolan-2-yl)-1-((2-(trimethylsilyl)ethoxy)methyl)-1H-pyrazolo[3,4-b]pyridine (Intermediate 26) instead of 4-(4,4,5,5-tetramethyl-1,3,2-dioxaborolan-2-yl)-1-((2-(trimethylsilyl)ethoxy)methyl)-1H-pyrrolo[2,3-b]pyridine (Intermediate 21) in Step A. Purification (SFC: Phenomenex-Cellulose-2 250×30 mm×10 μm column (eluent: 10% to 15% (v/v) Hexane-IPA)) afforded the title compound: MS (ESI): mass calcd. for C₂₁H₁₈FN₅ 359.2; m/z found 360.1 [M+H]⁺; ¹H NMR (400 MHz, CDCl₃): δ 7.63 (s, 1H), 7.45-7.39 (m, 2H), 7.02-6.95 (m, 2H), 6.87 (s, 1H), 4.99 (s, 1H), 3.77 (s, 1H), 2.69 (s, 3H), 2.40-2.35 (m, 1H), 2.12-2.03 (m, 2H), 1.95-1.90 (m, 1H), 1.66-1.41 (m, 2H); and (4*S,7*R)-2-(4-fluorophenyl)-3-(6-methyl-1H-pyrazolo[3,4-b]pyridin-4-yl)-4,5,6,7-tetrahydro-4,7-methanopyrazolo[1,5-a]pyridine (Example 365).

### Example 365: (4*S,7*R)-2-(4-Fluorophenyl)-3-(6-methyl-1H-pyrazolo[3,4-b]pyridin-4-yl)-4,5,6,7-tetrahydro-4,7-methanopyrazolo[1,5-a]pyridine

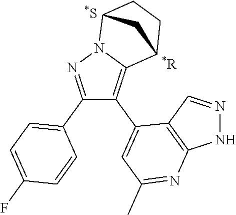 [SMILES: Cc1cc(-c2c(-c3ccc(F)cc3)nn3c2[C@H]2CC[C@@H]3C2)c2cn[nH]c2n1]

The title compound was isolated by SFC purification of Example 364. MS (ESI): mass calcd. for C₂₁H₁₈FN₅ 359.2; m/z found 360.2 [M+H]⁺. ¹H NMR (400 MHz, DMSO-d₆): δ 13.36 (br.s., 1H), 7.37-7.32 (m, 3H), 7.13-7.08 (m, 2H), 6.87 (s, 1H), 5.00 (s, 1H), 3.70 (br.s., 1H), 2.53 (s, 3H), 2.27-2.22 (m, 1H), 2.04-1.98 (m, 2H), 1.92-1.87 (m, 1H), 1.35-1.24 (m, 2H).

### Example 366: 2-(5-Fluoropyridin-2-yl)-6,6-bis(methyl-d₃)-3-(1H-pyrazolo[3,4-b]pyridin-4-yl)-4,5,6,7-tetrahydropyrazolo[1,5-a]pyridin-4-ol

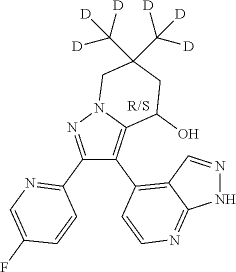 [SMILES: [2H]C([2H])([2H])C1(C([2H])([2H])[2H])CC(O)c2c(-c3ccnc4[nH]ncc34)c(-c3ccc(F)cn3)nn2C1]

Step A: 4-(4-Chloro-2-(5-fluoropyridin-2-yl)-6,6-bis(methyl-d₃)-4,5,6,7-tetrahydropyrazolo[1,5-a]pyridin-3-yl)-1H-pyrazolo[3,4-b]pyridine. NCS (149 mg, 1.12 mmol) was added to a 0° C. (ice/water) solution consisting of 4-(2-(5-fluoropyridin-2-yl)-6,6-bis(methyl-d₃)-4,5,6,7-tetrahydropyrazolo[1,5-a]pyridin-3-yl)-1H-pyrazolo[3,4-b]pyridine (Example 283) (180 mg, 0.489 mmol) and DMF (1.8 mL). The mixture was stirred at room-temperature for 3 hours. The mixture was concentrated to dryness under reduced pressure. The resulting residue was purified by FCC (petroleum ether:ethyl acetate) to afford the product (12.1 mg, 6%) as a white solid. MS (ESI): mass calcd. for C₂₀H₁₂ClD₆FN₆ 402.2 m/z found 403.0 [M+H]⁺. ¹H NMR (400 MHz, DMSO-d₆) δ 13.68-13.41 (m, 1H), 8.47 (d, J=4.8 Hz, 1H), 8.19 (d, J=2.8 Hz, 1H), 7.86-7.79 (m, 1H), 7.77-7.65 (m, 1H), 7.24 (d, J=4.5 Hz, 1H), 5.76 (s, 1H), 4.24-4.09 (m, 1H), 4.05-3.95 (m, 1H), 2.54 (s, 1H), 2.42-2.37 (m, 1H), 2.11-1.98 (m, 1H).

Step B: 2-(5-Fluoropyridin-2-yl)-6,6-bis(methyl-d₃)-3-(1H-pyrazolo[3,4-b]pyridin-4-yl)-4,5,6,7-tetrahydropyrazolo[1,5-a]pyridin-4-ol. 4-(4-Chloro-2-(5-fluoropyridin-2-yl)-6,6-bis(methyl-d₃)-4,5,6,7-tetrahydropyrazolo[1,5-a]pyridin-3-yl)-1H-pyrazolo[3,4-b]pyridine (12 mg, 0.030 mmol), DMF (0.5 mL) and H₂O (0.5 mL) were added to a 25 mL reaction flask. The resultant mixture was stirred at room-temperature for 5 hours. Then the mixture was concentrated to dryness under reduced pressure. The resulting residue was purified by preparative HPLC using a Boston Green ODS 150×30 mm×5 μm column (eluent: 36% to 66% (v/v) CH₃CN and H₂O with (0.225% HCOOH)) to afford pure product, the product was suspended in water (10 mL), the mixture frozen using dry ice/acetone, and then lyophilized to dryness to afford the title compound (5.6 mg, 48%) as a white solid. MS (ESI): mass calcd. for C₂₀H₁₃D₆FN₆O 384.2; m/z found 385.3 [M+H]⁺. ¹H NMR (400 MHz, DMSO-d₆) δ 13.40 (s, 1H), 8.42 (d, J=4.8 Hz, 1H), 8.19 (d, J=2.8 Hz, 1H), 7.83-7.66 (m, 2H), 7.34 (s, 1H), 7.24 (d, J=4.8 Hz, 1H), 5.05 (d, J=6.5 Hz, 1H), 4.99-4.94 (m, 1H), 4.09-3.87 (m, 2H), 1.98-1.93 (m, 1H), 1.68-1.63 (m, 1H).

### Example 367: 6-(5-Fluoro-2-pyridyl)-2,2-dimethyl-7-pyrazolo[1,5-a]pyridin-5-yl-3H-pyrazolo[5,1-b]oxazole

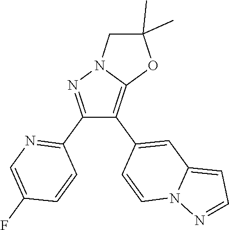 [SMILES: CC1(C)Cn2nc(-c3ccc(F)cn3)c(-c3ccn4nccc4c3)c2O1]

The title compound was prepared in a manner analogous to Example 1, Step A, except using 5-(4,4,5,5-tetramethyl-1,3,2-dioxaborolan-2-yl)pyrazolo[1,5-a]pyridine (Intermediate 224) instead of 4-(4,4,5,5-tetramethyl-1,3,2-dioxaborolan-2-yl)-1-((2-(trimethylsilyl)ethoxy)methyl)-1H-pyrrolo[2,3-b]pyridine (Intermediate 21) and 7-bromo-6-(5-fluoropyridin-2-yl)-2,2-dimethyl-2,3-dihydropyrazolo[5,1-b]oxazole (Intermediate 164) instead of 3-bromo-2-(5-fluoropyridin-2-yl)-6,7-dihydro-4H-pyrazolo[5,1-c][1,4]oxazine (Intermediate 37). MS (ESI): mass calcd. for C₁₉H₁₆FN₅O, 349.1; m/z found 350.1 [M+H]⁺. ¹H NMR (400 MHz, DMSO-d₆): δ 8.64-8.40 (m, 2H), 7.92 (d, J=2.0 Hz, 1H), 7.87-7.77 (m, 2H), 7.54-7.46 (m, 1H), 6.70-6.64 (m, 1H), 6.51 (d, J=1.6 Hz, 1H), 4.26 (s, 2H), 1.68 (s, 6H).

### Example 368: 6-(5-Fluoro-2-pyridyl)-2,2-dimethyl-7-(6-methyl-1H-pyrazolo[3,4-b]pyridin-4-yl)-3H-pyrazolo[5,1-b]oxazole

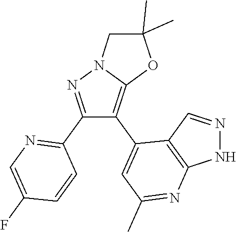 [SMILES: Cc1cc(-c2c(-c3ccc(F)cn3)nn3c2OC(C)(C)C3)c2cn[nH]c2n1]

The title compound was prepared in a manner analogous to Example 1, except using 1-(4-methoxybenzyl)-6-methyl-4-(4,4,5,5-tetramethyl-1,3,2-dioxaborolan-2-yl)-1H-pyrazolo[3,4-b]pyridine (Intermediate 217) instead of 4-(4,4,5,5-tetramethyl-1,3,2-dioxaborolan-2-yl)-1-((2-(trimethylsilyl)ethoxy)methyl)-1H-pyrrolo[2,3-b]pyridine (Intermediate 21) and 7-bromo-6-(5-fluoropyridin-2-yl)-2,2-dimethyl-2,3-dihydropyrazolo[5,1-b]oxazole (Intermediate 164) instead of 3-bromo-2-(5-fluoropyridin-2-yl)-6,7-dihydro-4H-pyrazolo[5,1-c][1,4]oxazine (Intermediate 37). MS (ESI): mass calcd. for C₁₉H₁₇FN₆O 364.1; m/z found 365.0 [M+H]⁺. ¹H NMR (400 MHz, DMSO-d₆): δ 13.26 (br s, 1H), 8.39-8.30 (m, 1H), 7.88-7.77 (m, 2H), 7.38 (s, 1H), 6.76 (s, 1H), 4.30 (s, 2H), 2.47 (s, 3H), 1.68 (s, 6H).

### Example 369: 2-(4-Fluorophenyl)-3-(4-pyridyl)-6,7-dihydro-4H-pyrazolo[5,1-c][1,4]oxazine

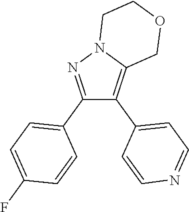 [SMILES: Fc1ccc(-c2nn3c(c2-c2ccncc2)COCC3)cc1]

The title compound was prepared in a manner analogous to Example 1, Step A, except using pyridin-4-ylboronic acid instead of 4-(4,4,5,5-tetramethyl-1,3,2-dioxaborolan-2-yl)-1-((2-(trimethylsilyl)ethoxy)methyl)-1H-pyrrolo[2,3-b]pyridine (Intermediate 21) and 3-bromo-2-(4-fluorophenyl)-6,7-dihydro-4H-pyrazolo[5,1-c][1,4]oxazine (Intermediate 65) instead of 3-bromo-2-(5-fluoropyridin-2-yl)-6,7-dihydro-4H-pyrazolo[5,1-c][1,4]oxazine (Intermediate 37). MS (ESI): mass calcd. for C₁₇H₁₄FN₃O 295.1; m/z found 296.1 [M+H]⁺. ¹H NMR (400 MHz, DMSO-d₆): δ 8.49 (d, J=6.0 Hz, 2H), 7.44-7.29 (m, 2H), 7.24-7.16 (m, 2H), 7.13-7.06 (m, 2H), 4.91 (s, 2H), 4.30-4.05 (m, 4H).

### Example 370: 3-(1-Ethyl-1H-pyrazolo[3,4-b]pyridin-5-yl)-2-(4-fluorophenyl)-7-methyl-6,7-dihydro-4H-pyrazolo[5,1-c][1,4]oxazine

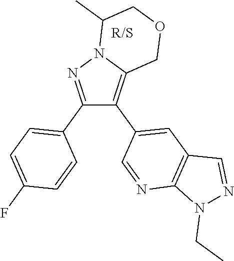 [SMILES: CCn1ncc2cc(-c3c(-c4ccc(F)cc4)nn4c3COCC4C)cnc21]

The title compound was prepared in manner analogous to Example 223, except using 3-bromo-2-(4-fluorophenyl)-7-methyl-6,7-dihydro-4H-pyrazolo[5,1-c][1,4]oxazine (Intermediate 76) instead of 3-bromo-2-(5-fluoropyridin-2-yl)-5,6-dihydro-4H-pyrrolo[1,2-b]pyrazole (Intermediate 39). (MS (ESI): mass calcd. for C₂₁H₂₀FN₅O 377.2; m/z found 378.2 [M+H]⁺. ¹H NMR (600 MHz, DMSO-d₆): δ 8.28 (d, J=2.0 Hz, 1H), 8.12 (s, 1H), 8.03 (d, J=2.1 Hz, 1H), 7.41-7.34 (m, 2H), 7.18-7.13 (m, 2H), 4.94-4.76 (m, 2H), 4.49 (q, J=7.2 Hz, 2H), 4.44-4.38 (m, 1H), 4.21 (dd, J=11.9, 4.3 Hz, 1H), 3.80 (dd, J=11.9, 6.5 Hz, 1H), 1.52 (d, J=6.5 Hz, 3H), 1.45 (t, J=7.2 Hz, 3H).

### Example 371: (S)-3-(1-Ethyl-1H-pyrazolo[3,4-b]pyridin-5-yl)-2-(5-fluoropyridin-2-yl)-6-methyl-6,7-dihydro-4H-pyrazolo[5,1-c][1,4]oxazine

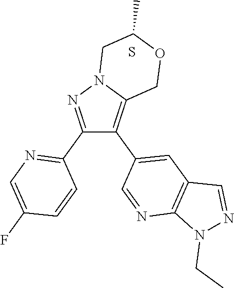

The title compound was prepared in manner analogous to Example 223, except using (S)-6-methyl-6,7-dihydro-4H-[1,2,3]oxadiazolo[4,3-c][1,4]oxazin-8-ium-3-olate (Intermediate 240) instead of 3-bromo-2-(5-fluoropyridin-2-yl)-5,6-dihydro-4H-pyrrolo[1,2-b]pyrazole (Intermediate 39). (MS (ESI): mass calcd. for C₂₀H₁₉FN₆O 378.2; m/z found 379.2 [M+H]⁺. ¹H NMR (600 MHz, DMSO-d₆): δ 8.38 (d, J=2.0 Hz, 1H), 8.35 (dt, J=3.0, 0.6 Hz, 1H), 8.11 (s, 1H), 8.06 (d, J=2.0 Hz, 1H), 7.91-7.86 (m, 1H), 7.76 (td, J=8.8, 3.0 Hz, 1H), 4.98-4.79 (m, 2H), 4.50 (qd, J=7.1, 2.4 Hz, 2H), 4.34 (dd, J=12.5, 3.2 Hz, 1H), 4.17-4.11 (m, 1H), 3.92-3.85 (m, 1H), 1.45 (t, J=7.2 Hz, 3H), 1.36 (d, J=6.2 Hz, 3H).

### Example 372: (*S)-2-(4-Fluorophenyl)-3-(3-methyl-1H-pyrazolo[3,4-b]pyridin-4-yl)-6-(trifluoromethyl)-6,7-dihydro-4H-pyrazolo[5,1-c][1,4]oxazine

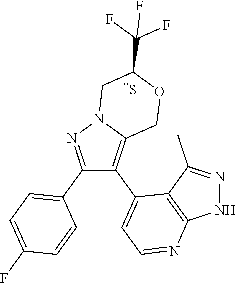

The title compound was prepared in a manner analogous to Example 1, Steps A-B, except using 3-bromo-2-(4-fluorophenyl)-6-(trifluoromethyl)-6,7-dihydro-4H-pyrazolo[5,1-c][1,4]oxazine (Intermediate 79) instead of 3-bromo-2-(5-fluoropyridin-2-yl)-6,7-dihydro-4H-pyrazolo[5,1-c][1,4]oxazine (Intermediate 37); and 3-methyl-4-(4,4,5,5-tetramethyl-1,3,2-dioxaborolan-2-yl)-1-((2-(trimethylsilyl)ethoxy)methyl)-1H-pyrazolo[3,4-b]pyridine (Intermediate 220) instead of 4-(4,4,5,5-tetramethyl-1,3,2-dioxaborolan-2-yl)-1-((2-(trimethylsilyl)ethoxy)methyl)-1H-pyrrolo[2,3-b]pyridine (Intermediate 21) in Step A. Purification (chiral SFC (DAICEL CHIRALPAK IG (250 mm×30 mm, 10 m) Column (isocratic elution: EtOH (containing 0.1% of 25% aq. NH₃): supercritical CO₂, 25%:75% to 25%:75% (v/v)) afforded the title compound: MS (ESI): mass calcd. for C₂₀H₁₅F₄N₅O 417.1; found 418.3 [M+H]⁺; ¹H NMR (400 MHz, DMSO-d₆): δ 13.33 (br s, 1H), 8.51 (d, J=4.5 Hz, 1H), 7.36-7.25 (m, 2H), 7.20-6.92 (m, 3H), 5.12-4.94 (m, 2H), 4.87-4.75 (m, 1H), 4.68-4.56 (m, 1H), 4.43-4.29 (m, 1H), 1.91 (d, J=15.3 Hz, 3H); and (*R)-2-(4-Fluorophenyl)-3-(3-methyl-1H-pyrazolo[3,4-b]pyridin-4-yl)-6-(trifluoromethyl)-6,7-dihydro-4H-pyrazolo[5,1-c][1,4]oxazine (Example 373).

### Example 373: (*R)-2-(4-Fluorophenyl)-3-(3-methyl-1H-pyrazolo[3,4-b]pyridin-4-yl)-6-(trifluoromethyl)-6,7-dihydro-4H-pyrazolo[5,1-c][1,4]oxazine

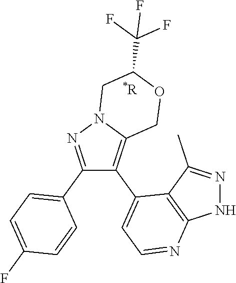

The title compound was isolated from chiral SFC purification of Example 372. MS (ESI): mass calcd. for C₂₀H₁₅F₄N₅O 417.1; found 418.3 [M+H]⁺. ¹H NMR (400 MHz, DMSO-d₆): δ 13.33 (br s, 1H), 8.51 (d, J=4.5 Hz, 1H), 7.34-7.24 (m, 2H), 7.13-6.94 (m, 3H), 5.13-4.96 (m, 2H), 4.88-4.76 (m, 1H), 4.68-4.55 (m, 1H), 4.44-4.25 (m, 1H), 1.91 (d, J=15.3 Hz, 3H).

### Example 374: (*S)-3-[6-(Difluoromethyl)-3-methyl-1H-pyrazolo[3,4-b]pyridin-4-yl]-2-(4-fluorophenyl)-6-(trifluoromethyl)-6,7-dihydro-4H-pyrazolo[5,1-c][1,4]oxazine

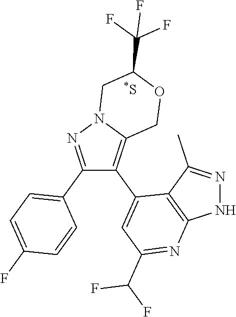

The title compound was prepared in a manner analogous to (*R)-2-(4-Fluorophenyl)-3-(6-methyl-1H-pyrazolo[3,4-d]pyrimidin-4-yl)-6-(trifluoromethyl)-6,7-dihydro-4H-pyrazolo[5,1-c][1,4]oxazine (Example 376), except using 4-chloro-6-(difluoromethyl)-3-methyl-1-((2-(trimethylsilyl)ethoxy)methyl)-1H-pyrazolo[3,4-b]pyridine (Intermediate 207) instead of 4-chloro-6-methyl-1-((2-(trimethylsilyl)ethoxy)methyl)-1H-pyrazolo[3,4-d]pyrimidine (Intermediate 212). SFC chiral separation (DAICEL CHIRALCEL OD-H (250 mm*30 mm, 5 um) (isocratic elution: MeOH (containing 0.1% of 25% aq. NH₃): supercritical CO₂, 15%: 85% to 15%: 85% (v/v))) afforded the title compound and (*R)-3-[6-(difluoromethyl)-3-methyl-1H-pyrazolo[3,4-b]pyridin-4-yl]-2-(4-fluorophenyl)-6-(trifluoromethyl)-6,7-dihydro-4H-pyrazolo[5,1-c][1,4]oxazine (Example 375). MS (ESI): mass calcd. for C₂₁H₁₅F₆N₅O 467.1; m/z found 468.4 [M+H]⁺. ¹H NMR (400 MHz, DMSO-d₆): δ 13.65 (br s, 1H), 7.40-7.22 (m, 3H), 7.07 (td, J=5.8, 56 Hz, 1H), 7.11-7.04 (m, 2H), 5.17-4.95 (m, 2H), 4.92-4.76 (m, 1H), 4.69-4.57 (m, 1H), 4.45-4.29 (m, 1H), 2.01-1.89 (m, 3H).

### Example 375: (*R)-3-[6-(Difluoromethyl)-3-methyl-1H-pyrazolo[3,4-b]pyridin-4-yl]-2-(4-fluorophenyl)-6-(trifluoromethyl)-6,7-dihydro-4H-pyrazolo[5,1-c][1,4]oxazine

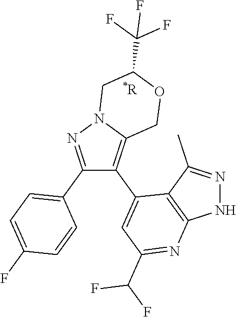

The title compound was isolated from chiral SFC purification of Example 374. MS (ESI): mass calcd. for C₂₁H₁₅F₆N₅O 467.1; m/z found 468.3 [M+H]⁺. ¹H NMR (400 MHz, DMSO-d₆): δ 13.65 (br s, 1H), 7.40-7.29 (m, 1H), 7.29-7.22 (m, 2H), 7.07 (td, J=5.84, 56 Hz, 1H), 7.11-7.04 (m, 2H), 5.14-4.96 (m, 2H), 4.91-4.76 (m, 1H), 4.63 (tt, J=3.8, 8.0 Hz, 1H), 4.42-4.29 (m, 1H), 1.98-1.90 (m, 3H).

### Example 376: (*R)-2-(4-Fluorophenyl)-3-(6-methyl-1H-pyrazolo[3,4-d]pyrimidin-4-yl)-6-(trifluoromethyl)-6,7-dihydro-4H-pyrazolo[5,1-c][1,4]oxazine

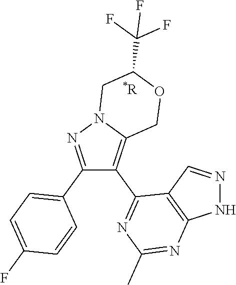

Step A. 2-(4-Fluorophenyl)-3-(6-methyl-1-((2-(trimethylsilyl)ethoxy)methyl)-1H-pyrazolo[3,4-d]pyrimidin-4-yl)-6-(trifluoromethyl)-6,7-dihydro-4H-pyrazolo[5,1-c][1,4]oxazine. 2-(4-Fluorophenyl)-3-(4,4,5,5-tetramethyl-1,3,2-dioxaborolan-2-yl)-6-(trifluoromethyl)-6,7-dihydro-4H-pyrazolo[5,1-c][1,4]oxazine (Intermediate 226, 380 mg, 0.922 mmol), 4-chloro-6-methyl-1-((2-(trimethylsilyl)ethoxy)methyl)-1H-pyrazolo[3,4-d]pyrimidine (Intermediate 212, 276 mg, 0.924 mmol), and K₃PO₄ (587 mg, 2.77 mmol) were added to a 40 mL flask and the resulting mixture dissolved in 1,4-dioxane (10 mL) and H₂O (2 mL). The resultant mixture was sparged with N₂ for 5 minutes and then treated with Pd(dtbpf)Cl₂ (60 mg, 0.092 mmol). The resultant mixture was sparged with N₂ for another 5 minutes and heated at 90° C. for 16 hours. The reaction mixture was cooled to room-temperature. The mixture was filtered, the filter cake was washed with ethyl acetate (5 mL×3). The filtrate was concentrated to dryness under reduced pressure. The resulting residue was purified by FCC (SiO₂, eluent: petroleum ether:ethyl acetate=1:0 to 1:1) to afford the desired product (150 mg, 29%) as a white solid. MS (ESI): mass calcd. for C₂₅H₂₈F₄N₆O₂Si 548.20; m/z found 549.2 [M+H]⁺.

Step B. (*R)-2-(4-Fluorophenyl)-3-(6-methyl-1H-pyrazolo[3,4-d]pyrimidin-4-yl)-6-(trifluoromethyl)-6,7-dihydro-4H-pyrazolo[5,1-c][1,4]oxazine. TFA (5 mL) was added to 2-(4-fluorophenyl)-3-(6-methyl-1-((2-(trimethylsilyl)ethoxy)methyl)-1H-pyrazolo[3,4-d]pyrimidin-4-yl)-6-(trifluoromethyl)-6,7-dihydro-4H-pyrazolo[5,1-c][1,4]oxazine (150 mg, 0.273 mmol) in a 50 mL round-bottomed flask. The mixture was stirred for 1 hour at room-temperature. The mixture was concentrated to dryness under reduced pressure to give the residue, which was suspended in NH₃/CH₃₀H (5 mL, 2 M) and stirred for 30 mins. The mixture was concentrated to dryness under reduced pressure to give the crude racemic product (100 mg) as a yellow solid. Purification (chiral SFC (DAICEL CHIRALCEL OD-H (250 mm×30 mm×5 m) (isocratic elution: MeOH (containing 0.1% of 25% aq. NH₃): supercritical CO₂, 25%: 75% to 25%: 75% (v/v)))) afforded the title compound to obtain the title compound and (*S)-2-(4-Fluorophenyl)-3-(6-methyl-1H-pyrazolo[3,4-d]pyrimidin-4-yl)-6-(trifluoromethyl)-6,7-dihydro-4H-pyrazolo[5,1-c][1,4]oxazine (Example 377). MS (ESI): mass calcd. for C₁₉H₁₄F₄N₆O 418.1; m/z found 419.2 [M+H]⁺. ¹H NMR (400 MHz, DMSO-d₆): δ 13.67 (br s, 1H), 7.48-7.42 (m, 2H), 7.25-7.19 (m, 2H), 6.69 (s, 1H), 5.39-5.31 (m, 1H), 5.30-5.23 (m, 1H), 5.17-5.05 (m, 1H), 4.60 (dd, J=3.6, 12.5 Hz, 1H), 4.40-4.30 (m, 1H), 2.69 (s, 3H).

### Example 377: (*S)-2-(4-Fluorophenyl)-3-(6-methyl-1H-pyrazolo[3,4-d]pyrimidin-4-yl)-6-(trifluoromethyl)-6,7-dihydro-4H-pyrazolo[5,1-c][1,4]oxazine

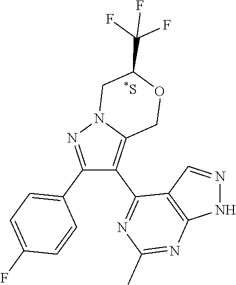

The title compound was isolated from chiral SFC purification of Example 376. MS (ESI): mass calcd. for C₁₉H₁₄F₄N₆O, 418.1; m/z found 419.1 [M+H]⁺. ¹H NMR (400 MHz, DMSO-d₆): δ 13.68 (br s, 1H), 7.48-7.42 (m, 2H), 7.25-7.19 (m, 2H), 6.69 (s, 1H), 5.38-5.31 (m, 1H), 5.30-5.23 (m, 1H), 5.16-5.05 (m, 1H), 4.60 (dd, J=3.6, 12.5 Hz, 1H), 4.39-4.29 (m, 1H), 2.69 (s, 3H).

### Example 378: 4-(2-(5-Fluoropyridin-2-yl)-6,6-dimethyl-6,7-dihydro-4H-pyrazolo[5,1-c][1,4]oxazin-3-yl)-5-methylpyridin-2-amine

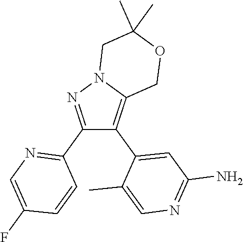 [SMILES: Cc1cnc(N)cc1-c1c(-c2ccc(F)cn2)nn2c1COC(C)(C)C2]

The title compound was prepared in a manner analogous to Example 1, Step A, except using lithium 2-(2-(5-fluoropyridin-2-yl)-6,6-dimethyl-6,7-dihydro-4H-pyrazolo[5,1-c][1,4]oxazin-3-yl)-2-hydroxy-4,4,5,5-tetramethyl-1,3,2-dioxaborolan-2-uide (Intermediate 105) instead of 3-bromo-2-(5-fluoropyridin-2-yl)-6,7-dihydro-4H-pyrazolo[5,1-c][1,4]oxazine (Intermediate 37) and 4-chloro-5-methylpyridin-2-amine instead of 4-(4,4,5,5-tetramethyl-1,3,2-dioxaborolan-2-yl)-1-((2-(trimethylsilyl)ethoxy)methyl)-1H-pyrrolo[2,3-b]pyridine (Intermediate 21). MS (ESI): mass calcd. for C₁₉H₂₀FN₅O 353.2; m/z found 354.2 [M+H]⁺. ¹H NMR (400 MHz, CD₃OD) δ 8.32 (d, J=2.9 Hz, 1H), 7.73 (t, J=0.9 Hz, 1H), 7.67-7.59 (m, 1H), 7.54 (td, J=8.6, 2.9 Hz, 1H), 6.46 (s, 1H), 4.89 (s, 1H), 4.59 (d, J=25.2 Hz, 1H), 4.08 (s, 2H), 1.74 (d, J=0.8 Hz, 3H), 1.40 (s, 6H). N—H protons not observed.

### Example 379: N-(4-(2-(5-Fluoropyridin-2-yl)-6,6-dimethyl-6,7-dihydro-4H-pyrazolo[5,1-c][1,4]oxazin-3-yl)-5-methylpyridin-2-yl)propionamide

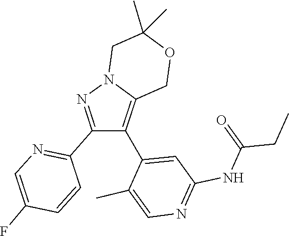 [SMILES: CCC(=O)Nc1cc(-c2c(-c3ccc(F)cn3)nn3c2COC(C)(C)C3)c(C)cn1]

To a solution of 4-(2-(5-fluoropyridin-2-yl)-6,6-dimethyl-6,7-dihydro-4H-pyrazolo[5,1-c][1,4]oxazin-3-yl)-5-methylpyridin-2-amine (Example 378, 11.5 mg, 0.0325 mmol), propionic acid (7.3 uL, 0.098 mmol), and DIPEA (22.4 uL, 0.13 mmol) was added HATU (18.6 mg, 0.0488 mmol). The reaction was heated to 50° C. and stirred until the reaction was complete by LCMS analysis. The crude reaction mixture was then filtered and purified (basic ACCQ-prep.

HPLC (20 mM NH₄OH in H₂O and neutral CH₃CN)) to afford the title compound as a white solid (8.7 mg, 65%). MS (ESI): mass calcd. for C₂₂H₂₄FN₅O₂ 409.2; m/z found 410.2 [M+H]+. ¹H NMR (400 MHz, CD₃OD) δ 8.26 (d, J=2.9 Hz, 1H), 8.11 (d, J=0.8 Hz, 1H), 7.89 (s, 1H), 7.70 (dd, J=8.8, 4.5 Hz, 1H), 7.55 (td, J=8.6, 2.9 Hz, 1H), 4.57 (s, 2H), 4.10 (s, 2H), 2.44 (q, J=7.6 Hz, 2H), 1.87 (s, 3H), 1.42 (s, 6H), 1.18 (t, J=7.6 Hz, 3H).

### Example 380: 5-[2-(5-Fluoro-2-pyridyl)-6,6-dimethyl-4,7-dihydropyrazolo[5,1-c][1,4]oxazin-3-yl]pyrazolo[1,5-a]pyridin-7-amine

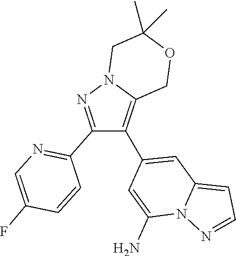 [SMILES: CC1(C)Cn2nc(-c3ccc(F)cn3)c(-c3cc(N)n4nccc4c3)c2CO1]

Step A. 2-(5-Fluoropyridin-2-yl)-3-(7-iodopyrazolo[1,5-a]pyridin-5-yl)-6,6-dimethyl-6,7-dihydro-4H-pyrazolo[5,1-c][1,4]oxazine. n-BuLi (0.06 mL, 0.14 mmol, 2.5 M) was added dropwise to a −78° C. consisting of 2-(5-fluoropyridin-2-yl)-6,6-dimethyl-3-(pyrazolo[1,5-a]pyridin-5-yl)-6,7-dihydro-4H-pyrazolo[5,1c][1,4]oxazine (Example 449, 40 mg, 0.11 mmol) and THF (1.5 mL). The resultant mixture was stirred at −78° C. for 30 min. Then 1,2-diiodoethane (37 mg, 0.13 mmol) and THF (0.5 mL) was added to the above mixture at −78° C.

The mixture was stirred at −78° C. for another 2 hours. The reaction mixture was quenched with sat. NH₄Cl (5 mL) and extracted with ethyl acetate (8 mL×3). The combined organic extracts were washed with brine (5 mL), dried over anhydrous Na₂SO₄, filtered, and concentrated to dryness under reduced pressure. The resulting residue was purified by preparative HPLC using a Boston Uni C18 40 mm×150 mm×5 μm column (eluent: 25% to 55% (v/v) CH₃CN and H₂O with 0.05% NH₃) to afford pure product (10 mg, 18.57%) as a white solid. MS (ESI): mass calcd. for C₂₀H₁₇FIN₅O 489.3 m/z; found 490.0 [M+H]⁺. ¹H NMR (400 MHz, CDCl₃): δ 8.41 (d, J=2.8 Hz, 1H), 8.04 (d, J=2.3 Hz, 1H), 7.63-7.62 (m, 1H), 7.41-7.35 (m, 2H), 7.17 (d, J=1.5 Hz, 1H), 6.71 (d, J=2.3 Hz, 1H), 4.90 (s, 2H), 4.07 (s, 2H), 1.44 (s, 6H).

Step B. tert-Butyl (5-(2-(5-fluoropyridin-2-yl)-6,6-dimethyl-6,7-dihydro-4H-pyrazolo[5,1-c][1,4]oxazin-3-yl)pyrazolo[1,5-a]pyridin-7-yl)carbamate. CuI (2 mg, 0.01 mmol) was added to a mixture consisting of 2-(5-fluoropyridin-2-yl)-3-(7-iodopyrazolo[1,5-a]pyridin-5-yl)-6,6-dimethyl-6,7-dihydro-4H-pyrazolo[5,1-c][1,4]oxazine (50 mg, 0.073 mmol, 71% purity), tert-butyl carbamate (25 mg, 0.21 mmol), N,N-dimethylethylenediamine (3 mg, 0.03 mmol), K₂CO₃ (20 mg, 0.15 mmol), and toluene (2 mL). The resultant mixture was stirred at 90° C. under N₂ for 12 hours. The mixture was filtered and the filtrate was concentrated to dryness under reduced pressure. The resulting residue was purified by preparative TLC (eluent: petroleum ether:ethyl acetate=1:1) to afford the title compound (60 mg, 59.46% purity) as a white solid. MS (ESI): mass calcd. for C₂₅H₂₇FN₆O₃ 478.2; m/z, found 479.5 [M+H]⁺.

Step C. 5-(2-(5-Fluoropyridin-2-yl)-6,6-dimethyl-6,7-dihydro-4H-pyrazolo[5,1-c][1,4]oxazin-3-yl)pyrazolo[1,5-a]pyridin-7-amine. TFA (1 mL) was added to a solution consisting of tert-butyl (5-(2-(5-fluoropyridin-2-yl)-6,6-dimethyl-6,7-dihydro-4H-pyrazolo[5,1-c][1,4]oxazin-3-yl)pyrazolo[1,5-a]pyridin-7-yl)carbamate (65 mg, 0.096 mmol, 71% purity) and dichloromethane (3 mL) at room-temperature. The resultant mixture was stirred at room-temperature for 1 hour. The mixture was concentrated to dryness under reduced pressure. The resulting residue was basified to pH˜8 by 25% NH₃ (aq) and purified by preparative HPLC using a Phenomenex Gemini-NX C18 75 mm×30 mm×3 m column (eluent: 30% to 60% (v/v) CH₃CN and H₂O with 0.05% NH₃+10 mM NH₄HCO₃) to afford pure product. The product was suspended in water (40 mL), the mixture frozen using dry ice/EtOH, and then lyophilized to dryness to afford the title compound (9 mg, 22%) as a white off solid. MS (ESI): mass calcd. for C₂₀H₁₉FN₆O 378.2; m/z found 379.2 [M+H]⁺. ¹H NMR (400 MHz, CDCl₃): δ 8.50 (br s, 1H), 7.97 (d, J=2.3 Hz, 1H), 7.54-7.38 (m, 1H), 7.34-7.27 (m, 1H), 6.84 (d, J=1.3 Hz, 1H), 6.43 (d, J=2.3 Hz, 1H), 5.84 (d, J=1.5 Hz, 1H), 5.53-4.78 (m, 2H), 4.90 (s, 2H), 4.09 (s, 2H), 1.43 (s, 6H).

### Example 381: 5-[2-(5-Fluoro-2-pyridyl)-6,6-dimethyl-4,7-dihydropyrazolo[5,1-c][1,4]oxazin-3-yl]pyrazolo[1,5-a]pyridin-3-amine

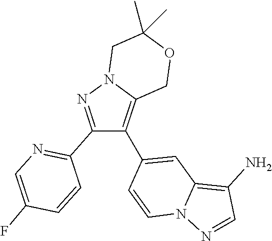 [SMILES: CC1(C)Cn2nc(-c3ccc(F)cn3)c(-c3ccn4ncc(N)c4c3)c2CO1]

3-(3-Bromopyrazolo[1,5-a]pyridin-5-yl)-2-(5-fluoropyridin-2-yl)-6,6-dimethyl-6,7-dihydro-4H-pyrazolo[5,1-c][1,4]oxazine (Example 383, product from Step A, 400 mg, 0.904 mmol), diphenylmethanimine (180 mg, 0.993 mmol), t-BuOK (203 mg, 1.81 mmol) were dissolved in 1,4-dioxane (5 mL). The resultant mixture was sparged with Ar for 5 minutes and then treated with Pd₂(dba)₃ (83 mg, 0.091 mmol), and BINAP (84 mg, 0.135 mmol). The mixture was sparged with Ar for another 5 minutes and heated at 90° C. for 16 hours. The reaction mixture was cooled to room-temperature. The resultant mixture was added HCl (6 M, 2 mL) and stirred at room-temperature for 2 hours. The reaction mixture was poured into water (10 mL) and extracted with ethyl acetate (10 mL×2). The aqueous phase was adjusted to pH>7 with aq.

NaOH and extracted with ethyl acetate (10 mL×3). The combined organic extracts were washed with brine (10 mL), dried over anhydrous Na₂SO₄, filtered, and concentrated to dryness under reduced pressure. The resulting residue was purified by SFC over DAICELCHIRALCEL OD-H 250 mm×30 mm, Sum (isocratic elution: EtOH (containing 0.1% of 25% aq. NH₃): supercritical CO₂, 55%: 45% to 55%: 45% (v/v)). The pure fractions were collected and the volatiles were removed under reduced pressure. The product was suspended in water (10 mL), the mixture frozen using dry ice/acetone, and then lyophilized to dryness to afford the title compound (25 mg, 7%) as a yellow solid. MS (ESI): mass calcd. for C₂₀H₁₉FN₆O 378.2; m/z found 379.1 [M+H]⁺. ¹H NMR (400 MHz, DMSO-d₆): δ 8.45 (d, J=2.86 Hz, 1H), 8.17 (d, J=7.27 Hz, 1H), 7.72-7.86 (m, 2H), 7.41-7.47 (m, 2H), 6.25-6.30 (m, 1H), 4.94 (s, 2H), 4.26 (s, 2H), 4.07 (s, 2H), 1.37 (s, 6H).

### Example 382: 2-(5-Fluoro-2-pyridyl)-6,6-dimethyl-3-(6-methylpyrazolo[1,5-a]pyridin-5-yl)-4,7-dihydropyrazolo[5,1-c][1,4]oxazine

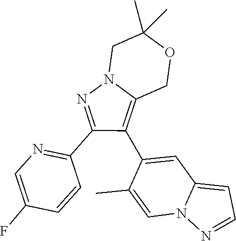 [SMILES: Cc1cn2nccc2cc1-c1c(-c2ccc(F)cn2)nn2c1COC(C)(C)C2]

The title compound was prepared in a manner analogous to Example 1, Step A, except using 5-bromo-6-methylpyrazolo[1,5-a]pyridine instead of Intermediate 37 and 2-(5-fluoropyridin-2-yl)-6,6-dimethyl-3-(4,4,5,5-tetramethyl-1,3,2-dioxaborolan-2-yl)-6,7-dihydro-4H-pyrazolo[5,1-c][1,4]oxazine (Intermediate 227) instead of 4-(4,4,5,5-tetramethyl-1,3,2-dioxaborolan-2-yl)-1-((2-(trimethylsilyl)ethoxy)methyl)-1H-pyrrolo[2,3-b]pyridine (Intermediate 21). MS (ESI): mass calcd. for C₂₁H₂₀FN₅O 377.2; m/z found 378.1 [M+H]⁺. ¹H NMR (400 MHz, DMSO-d₆): δ 8.52 (s, 1H), 8.31 (d, J=2.86 Hz, 1H), 7.86-7.95 (m, 2H), 7.68-7.77 (m, 1H), 7.53 (s, 1H), 6.44-6.51 (m, 1H), 4.84 (d, J=15.85 Hz, 1H), 4.54 (d, J=15.74 Hz, 1H), 4.03-4.15 (m, 2H), 1.84 (s, 3H), 1.38 (s, 3H), 1.33 (s, 3H).

### Example 383: 2-(5-Fluoro-2-pyridyl)-6,6-dimethyl-3-(3-methylpyrazolo[1,5-a]pyridin-5-yl)-4,7-dihydropyrazolo[5,1-c][1,4]oxazine

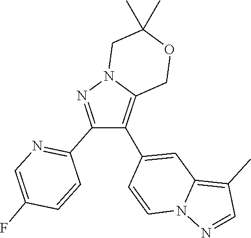 [SMILES: Cc1cnn2ccc(-c3c(-c4ccc(F)cn4)nn4c3COC(C)(C)C4)cc12]

Step A. 3-(3-Bromopyrazolo[1,5-a]pyridin-5-yl)-2-(5-fluoropyridin-2-yl)-6,6-dimethyl-6,7-dihydro-4H-pyrazolo[5,1-c][1,4]oxazine. 2-(5-Fluoropyridin-2-yl)-6,6-dimethyl-3-(pyrazolo[1,5-a]pyridin-5-yl)-6,7-dihydro-5H-pyrazolo[5,1-b][1,3]oxazine (Example 449, 900 mg, 2.48 mmol), 1-bromopyrrolidine-2,5-dione (530 mg, 2.98 mmol), and dichloromethane (20 mL) were added to a 40 mL tube. The mixture was stirred at room-temperature for 3 hours. The reaction mixture was quenched with aq·Na₂S₂O₃ (50 mL) and extracted with dichloromethane (20 mL×3). The combined organic extracts were dried over anhydrous Na₂SO₄, filtered and concentrated to dryness. The resulting residue was purified by FCC (SiO₂, eluent: petroleum ether:ethyl acetate=1:0 to 3:1) to afford the title compound (900 mg, 70%) as a brown solid. MS (ESI): mass calcd. for C₂₀H₁₇BrFN₅O 441.1; m/z found 442.1 [M+H]⁺.

Step B. 2-(5-Fluoropyridin-2-yl)-6,6-dimethyl-3-(3-methylpyrazolo[1,5-a]pyridin-5-yl)-6,7-dihydro-4H-pyrazolo[5,1-c][1,4]oxazine. 3-(3-Bromopyrazolo[1,5-a]pyridin-5-yl)-2-(5-fluoropyridin-2-yl)-6,6-dimethyl-6,7-dihydro-4H-pyrazolo[5,1-c][1,4]oxazine (300 mg, 0.678 mmol), 2,4,6-trimethyl-1,3,5,2,4,6-trioxatriborinane (0.2 mL, 0.7 mmol, 50% in THF), K₂CO₃ (2.0 mL, 4 mmol, 2 M in water), and 1,4-dioxane (10 mL) were combined. The mixture was sparged with Ar for 5 minutes and then treated with Pd(dppf)Cl₁·CH₂Cl₂ (55 mg, 0.067 mmol).

The mixture was sparged with Ar for another 5 minutes and the resultant mixture was heated at 80° C. for 2 hours. The reaction mixture was cooled to room-temperature. The suspension was quenched with NaHCO₃(20 mL) and extracted with ethyl acetate (30 mL×3). The combined organic extracts were washed with brine, dried over anhydrous Na₂SO₄, filtered, and concentrated to dryness under reduced pressure. The resulting residue was purified by preparative HPLC using a Boston Green ODS 150 mm×30 mm×5 μm column (eluent: 45% to 75% (v/v) CH₃CN and H₂O with 0.225% HCOOH) to afford pure product (41.6 mg, 16%) as a white solid. MS (ESI): mass calcd. for C₂₁H₂₀FN₅O 377.2; m/z found 378.1 [M+H]⁺. ¹H NMR (400 MHz, DMSO-d₆): δ 8.30-8.52 (m, 2H), 7.70-7.88 (m, 3H), 7.46-7.52 (m, 1H), 6.50 (dd, J=1.79, 7.27 Hz, 1H), 4.95 (s, 2H), 4.07 (s, 2H), 2.25 (s, 3H), 1.37 (s, 6H).

### Example 384: 3-(3-Chloropyrazolo[1,5-a]pyridin-5-yl)-2-(5-fluoropyridin-2-yl)-6,6-dimethyl-6,7-dihydro-4H-pyrazolo[5,1-c][1,4]oxazine

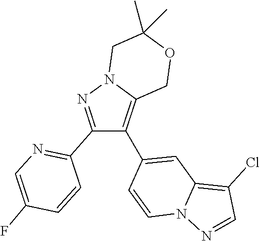 [SMILES: CC1(C)Cn2nc(-c3ccc(F)cn3)c(-c3ccn4ncc(Cl)c4c3)c2CO1]

To a cooled (0° C.) solution of 2-(5-fluoropyridin-2-yl)-6,6-dimethyl-3-(pyrazolo[1,5-a]pyridin-5-yl)-6,7-dihydro-4H-pyrazolo[5,1-c][1,4]oxazine (Example 140, 60 mg, 0.17 mmol) in MeCN/DCM (3 mL, v/v 1:2) was added N-chlorosuccinimide (24 mg, 0.18 mmol). Additional N-chlorosuccinimide (12 mg, 0.090 mmol) was added to the reaction mixture and the reaction mixture was heated to 30-35° C. for 7 hours. The reaction mixture was diluted with DCM and brine. The aqueous layer was extracted with DCM (3×). The combined organic layers were dried (Na₂SO₄), filtered, concentrated under reduced pressure. The resulting residue was purified (Basic AQQU Prep with ACN/NH₄OH 0-100% over 25 min) to afford the title compound (33 mg, 50%). MS (ESI): mass calcd. for C₂₀H₁₇ClFN₅O 397.1; m/z found 398.1 [M+H]⁺. ¹H NMR (600 MHz, DMSO-d₆): δ 8.58 (dd, J=7.2, 0.9 Hz, 1H), 8.45 (d, J=3.0 Hz, 1H), 8.14 (s, 1H), 7.97-7.88 (m, 1H), 7.81 (td, J=8.8, 3.0 Hz, 1H), 7.51 (dd, J=2.0, 0.9 Hz, 1H), 6.73 (dd, J=7.2, 2.0 Hz, 1H), 4.98 (s, 2H), 4.10 (s, 2H), 1.40 (s, 6H).

### Example 385: 3-([1,2,4]Triazolo[1,5-a]pyridin-7-yl)-2-(5-fluoropyridin-2-yl)-6,6-dimethyl-6,7-dihydro-4H-pyrazolo[5,1-c][1,4]oxazine

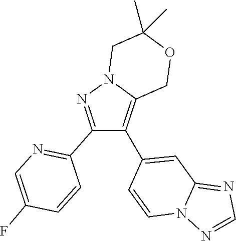 [SMILES: CC1(C)Cn2nc(-c3ccc(F)cn3)c(-c3ccn4ncnc4c3)c2CO1]

The title compound was prepared in manner analogous to Example 223, except using lithium 2-(2-(5-fluoropyridin-2-yl)-6,6-dimethyl-6,7-dihydro-4H-pyrazolo[5,1-c][1,4]oxazin-3-yl)-2-hydroxy-4,4,5,5-tetramethyl-1,3,2-dioxaborolan-2-uide (Intermediate 105) instead of 3-bromo-2-(5-fluoropyridin-2-yl)-5,6-dihydro-4H-pyrrolo[1,2-b]pyrazole (Intermediate 39) and using 7-bromo-[1,2,4]triazolo[1,5-a]pyridine instead of 1-ethyl-5-(4,4,5,5-tetramethyl-1,3,2-dioxaborolan-2-yl)-1H-pyrazolo[3,4-b]pyridine (Intermediate 31). MS (ESI): mass calcd. for C₁₉H₁₇FN₆O 364.1; m/z found 365.1 [M+H]⁺. ¹H NMR (600 MHz, DMSO-d₆): δ 8.81 (dd, J=7.1, 0.9 Hz, 1H), 8.48 (s, 1H), 8.46-8.41 (m, 1H), 7.92 (ddd, J=8.8, 4.6, 0.7 Hz, 1H), 7.81 (td, J=8.8, 3.0 Hz, 1H), 7.77 (dd, J=1.9, 0.9 Hz, 1H), 6.95 (dd, J=7.1, 1.8 Hz, 1H), 5.00 (s, 2H), 4.11 (s, 2H), 1.40 (s, 6H).

### Example 386: 2-(5-Fluoropyridin-2-yl)-6,6-dimethyl-3-(2-methyl-[1,2,4]triazolo[1,5-a]pyridin-7-yl)-6,7-dihydro-4H-pyrazolo[5,1-c][1,4]oxazine

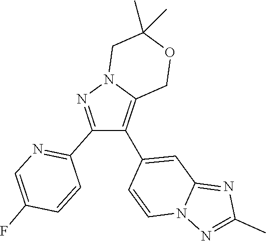 [SMILES: Cc1nc2cc(-c3c(-c4ccc(F)cn4)nn4c3COC(C)(C)C4)ccn2n1]

The title compound was prepared in manner analogous to Example 223, except using lithium 2-(2-(5-fluoropyridin-2-yl)-6,6-dimethyl-6,7-dihydro-4H-pyrazolo[5,1-c][1,4]oxazin-3-yl)-2-hydroxy-4,4,5,5-tetramethyl-1,3,2-dioxaborolan-2-uide (Intermediate 105) instead of 3-bromo-2-(5-fluoropyridin-2-yl)-5,6-dihydro-4H-pyrrolo[1,2-b]pyrazole (Intermediate 39) and using 7-bromo-2-methyl-[1,2,4]triazolo[1,5-a]pyridine instead of 1-ethyl-5-(4,4,5,5-tetramethyl-1,3,2-dioxaborolan-2-yl)-1H-pyrazolo[3,4-b]pyridine (Intermediate 31). MS (ESI): mass calcd. for C₂₀H₁₉FN₆O 378.2; m/z found 379.1 [M+H]⁺. ¹H NMR (600 MHz, DMSO-d₆): δ 8.68 (dd, J=7.0, 0.9 Hz, 1H), 8.44 (d, J=3.0 Hz, 1H), 7.90 (ddd, J=8.8, 4.6, 0.7 Hz, 1H), 7.81 (td, J=8.8, 3.0 Hz, 1H), 7.59 (dd, J=1.9, 0.9 Hz, 1H), 6.87 (dd, J=7.1, 1.9 Hz, 1H), 4.98 (s, 2H), 4.10 (s, 2H), 2.48 (s, 3H), 1.39 (s, 6H).

### Example 387: 2-(5-Fluoropyridin-2-yl)-6,6-dimethyl-3-(pyrazolo[1,5-a]pyrimidin-5-yl)-6,7-dihydro-4H-pyrazolo[5,1-c][1,4]oxazine

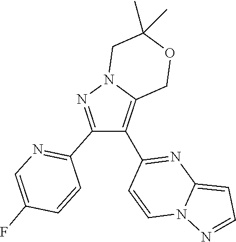 [SMILES: CC1(C)Cn2nc(-c3ccc(F)cn3)c(-c3ccn4nccc4n3)c2CO1]

The title compound was prepared in manner analogous to Example 223, except using lithium 2-(2-(5-fluoropyridin-2-yl)-6,6-dimethyl-6,7-dihydro-4H-pyrazolo[5,1-c][1,4]oxazin-3-yl)-2-hydroxy-4,4,5,5-tetramethyl-1,3,2-dioxaborolan-2-uide (Intermediate 105) instead of 3-bromo-2-(5-fluoropyridin-2-yl)-5,6-dihydro-4H-pyrrolo[1,2-b]pyrazole (Intermediate 39) and using 5-bromopyrazolo[1,5-a]pyrimidine instead of 1-ethyl-5-(4,4,5,5-tetramethyl-1,3,2-dioxaborolan-2-yl)-1H-pyrazolo[3,4-b]pyridine (Intermediate 31). MS (ESI): mass calcd. for C₁₉H₁₇FN₆O 364.1; m/z found 365.1 [M+H]⁺. ¹H NMR (600 MHz, DMSO-d₆): δ 8.86 (dd, J=7.4, 0.9 Hz, 1H), 8.59 (dt, J=2.9, 0.6 Hz, 1H), 8.17 (d, J=2.3 Hz, 1H), 7.93 (ddd, J=8.7, 4.7, 0.7 Hz, 1H), 7.89 (td, J=8.7, 2.9 Hz, 1H), 6.81 (d, J=7.4 Hz, 1H), 6.63 (dd, J=2.3, 0.9 Hz, 1H), 5.12 (s, 2H), 4.13 (s, 2H), 1.38 (s, 6H).

### Example 388: 3-(3-Chloro-1H-pyrazolo[3,4-b]pyridin-4-yl)-2-(5-fluoropyridin-2-yl)-6,6-dimethyl-6,7-dihydro-4H-pyrazolo[5,1-c][1,4]oxazine

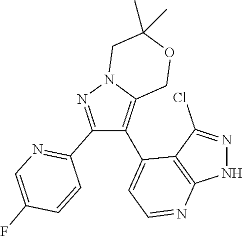 [SMILES: CC1(C)Cn2nc(-c3ccc(F)cn3)c(-c3ccnc4[nH]nc(Cl)c34)c2CO1]

The title compound was prepared in a manner analogous to Example 224, except using 2-(5-fluoropyridin-2-yl)-6,6-dimethyl-3-(1H-pyrazolo[3,4-b]pyridin-4-yl)-6,7-dihydro-4H-pyrazolo[5,1-c][1,4]oxazine (Example 150) instead of Example 9. MS (ESI): mass calcd. for C₁₉H₁₆ClFN₆O 398.1; m/z found 399.0 [M+H]⁺. ¹H NMR (400 MHz, CD₃OD): δ 8.52 (d, J=4.7 Hz, 1H), 8.05 (d, J=2.9 Hz, 1H), 7.85-7.73 (m, 1H), 7.51 (td, J=8.7, 2.9 Hz, 1H), 7.12 (d, J=4.7 Hz, 1H), 4.88 (s, 1H), 4.66 (s, 1H), 4.13 (d, J=1.4 Hz, 2H), 1.43 (d, J=11.9 Hz, 6H). N—H proton not observed.

### Example 389: 3-(5-Fluoro-1H-pyrazolo[3,4-b]pyridin-4-yl)-2-(5-fluoropyridin-2-yl)-6,6-dimethyl-6,7-dihydro-4H-pyrazolo[5,1-c][1,4]oxazine

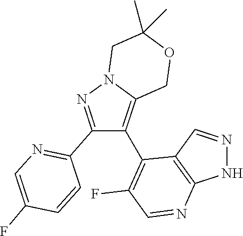 [SMILES: CC1(C)Cn2nc(-c3ccc(F)cn3)c(-c3c(F)cnc4[nH]ncc34)c2CO1]

The title compound was prepared in a manner analogous to Example 1, Steps A-B, except using lithium 2-(2-(5-fluoropyridin-2-yl)-6,6-dimethyl-6,7-dihydro-4H-pyrazolo[5,1-c][1,4]oxazin-3-yl)-2-hydroxy-4,4,5,5-tetramethyl-1,3,2-dioxaborolan-2-uide (Intermediate 105) instead of 3-bromo-2-(5-fluoropyridin-2-yl)-6,7-dihydro-4H-pyrazolo[5,1-c][1,4]oxazine (Intermediate 37) and 5-fluoro-4-iodo-1-((2-(trimethylsilyl)ethoxy)methyl)-1H-pyrazolo[3,4-b]pyridine (Intermediate 204) instead of 4-(4,4,5,5-tetramethyl-1,3,2-dioxaborolan-2-yl)-1-((2-(trimethylsilyl)ethoxy)methyl)-1H-pyrrolo[2,3-b]pyridine (Intermediate 21) in Step A. MS (ESI): mass calcd. for C₁₉H₁₆F₂N₆O 382.1; m/z found 383.1 [M+H]⁺. ¹H NMR (400 MHz, CD₃OD): δ 8.47 (d, J=2.8 Hz, 1H), 8.11 (dt, J=2.9, 0.7 Hz, 1H), 7.91-7.78 (m, 1H), 7.59 (td, J=8.6, 2.9 Hz, 1H), 7.47 (s, 1H), 4.79 (s, 2H), 4.24-4.05 (m, 2H), 1.44 (s, 6H). N—H proton not observed.

### Example 390: 3-[6-(Difluoromethyl)-1H-pyrazolo[3,4-b]pyridin-4-yl]-2-(5-fluoro-2-pyridyl)-6,6-dimethyl-4,7-dihydropyrazolo[5,1-c][1,4]oxazine

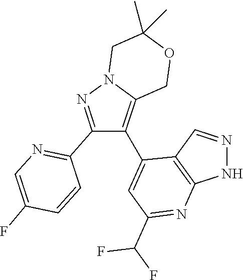 [SMILES: CC1(C)Cn2nc(-c3ccc(F)cn3)c(-c3cc(C(F)F)nc4[nH]ncc34)c2CO1]

The title compound was prepared in a manner analogous to Example 1, Steps A-B, except using 3-bromo-2-(5-fluoropyridin-2-yl)-6,6-dimethyl-6,7-dihydro-4H-pyrazolo[5,1-c][1,4]oxazine (Intermediate 101) instead of 3-bromo-2-(5-fluoropyridin-2-yl)-6,7-dihydro-4H-pyrazolo[5,1-c][1,4]oxazine (Intermediate 37) and 6-(difluoromethyl)-4-(4,4,5,5-tetramethyl-1,3,2-dioxaborolan-2-yl)-1-((2-(trimethylsilyl)ethoxy)methyl)-1H-pyrazolo[3,4-b]pyridine (Intermediate 221) instead of 4-(4,4,5,5-tetramethyl-1,3,2-dioxaborolan-2-yl)-1-((2-(trimethylsilyl)ethoxy)methyl)-1H-pyrrolo[2,3-b]pyridine (Intermediate 21) in Step A. MS (ESI): mass calcd. for C₂₀H₁₇F₃N₆O 414.1; m/z found 415.1 [M+H]⁺. ¹H NMR (400 MHz, CDCl₃): δ 8.16 (d, J=2.9 Hz, 1H), 7.58 (dd, J=4.3, 8.7 Hz, 1H), 7.42 (s, 1H), 7.30 (dd, J=2.9, 8.2 Hz, 1H), 7.19 (s, 1H), 6.85-6.53 (m, 1H), 4.82 (s, 2H), 4.07 (s, 2H), 1.40 (s, 6H).

### Example 391: 2-(5-Fluoro-2-pyridyl)-6,6-dimethyl-3-[6-(3,3,3-trifluoropropyl)-1H-pyrazolo[3,4-b]pyridin-4-yl]-4,7-dihydropyrazolo[5,1-c][1,4]oxazine

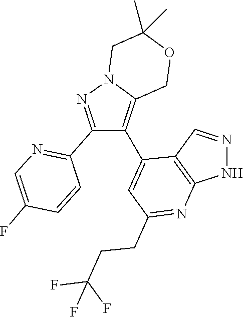 [SMILES: CC1(C)Cn2nc(-c3ccc(F)cn3)c(-c3cc(CCC(F)(F)F)nc4[nH]ncc34)c2CO1]

Step A. 4-(2-(5-Fluoropyridin-2-yl)-6,6-dimethyl-6,7-dihydro-4H-pyrazolo[5,1-c][1,4]oxazin-3-yl)-1-((2-(trimethylsilyl)ethoxy)methyl)-1H-pyrazolo[3,4-b]pyridine 7-oxide. m-CPBA (1.31 g, 80% purity, 6.06 mmol) was added to a solution of 2-(5-fluoropyridin-2-yl)-6,6-dimethyl-3-(1-((2-(trimethylsilyl)ethoxy)methyl)-1H-pyrazolo[3,4-b]pyridin-4-yl)-6,7-dihydro-4H-pyrazolo[5,1-c][1,4]oxazine (Example 150 product from Step A, 1.00 g, 2.02 mmol) and dichloromethane (15 mL) and the mixture was stirred at r.t. for 2 hours. The reaction mixture was poured into sat. NaHSO₃ (50 mL) and extracted with dichloromethane (50 mL×3). The combined organic extracts were washed with brine (50 mL), dried over anhydrous Na₂SO₄, filtered, and concentrated to dryness under reduced pressure. The resulting residue was purified (FCC, SiO₂, ethyl acetate:methanol=10:1, compound P1: petroleum ether:ethyl acetate=0:1, R^{f}=0.2) to afford the title compound (400 mg, 38%) as a yellow solid. MS (ESI): mass calcd. for C₂₅H₃₁FN₆O₃Si 510.2; m/z found 511.1 [M+H]⁺.

Step B. 3-(6-Chloro-1-((2-(trimethylsilyl)ethoxy)methyl)-1H-pyrazolo[3,4-b]pyridin-4-yl)-2-(5-fluoropyridin-2-yl)-6,6-dimethyl-6,7-dihydro-4H-pyrazolo[5,1-c][1,4]oxazine. MsCl (5.19 g, 45.3 mmol) was added to a solution consisting of 4-(2-(5-fluoropyridin-2-yl)-6,6-dimethyl-6,7-dihydro-4H-pyrazolo[5,1-c][1,4]oxazin-3-yl)-1-((2-(trimethylsilyl)ethoxy)methyl)-1H-pyrazolo[3,4-b]pyridine 7-oxide (400 mg, 0.783 mmol) and DMF (6 mL). The resultant mixture was then stirred at 85° C. for 2 hours. The reaction mixture was cooled to room-temperature, poured into sat. NaHCO₃(10 mL) and extracted with dichloromethane (10 mL×3). The combined organic extracts were washed with brine (10 mL), dried over anhydrous Na₂SO₄, filtered, and concentrated to dryness under reduced pressure. The resulting residue was purified by FCC (SiO₂, eluent: petroleum ether:ethyl acetate=1:0 to 1:1) to afford the title compound (180 mg, 35%) as a yellow solid. MS (ESI): mass calcd. for C₂₅H₃₀ClFN₆O₂Si 528.2; m/z found 529.1 [M+H]⁺.

Step C. 2-(5-Fluoropyridin-2-yl)-6,6-dimethyl-3-(6-(3,3,3-trifluoropropyl)-1-((2-(trimethylsilyl)ethoxy)methyl)-1H-pyrazolo[3,4-b]pyridin-4-yl)-6,7-dihydro-4H-pyrazolo[5,1-c][1,4]oxazine. 3-(6-Chloro-1-((2-(trimethylsilyl)ethoxy)methyl)-1H-pyrazolo[3,4-b]pyridin-4-yl)-2-(5-fluoropyridin-2-yl)-6,6-dimethyl-6,7-dihydro-4H-pyrazolo[5,1-c][1,4]oxazine (180 mg, 0.340 mmol), potassium trifluoro(3,3,3-trifluoropropyl)borate (70 mg, 0.34 mmol), and Cs₂CO₃ (333 mg, 1.02 mmol) were dissolved toluene (10 mL) and H₂O (1 mL). The mixture was sparged with N₂ for 5 minutes and then treated with Pd(Amphos)₂Cl₂ (30 mg, 0.42 mmol). The resultant mixture was sparged with N₂ for another 5 minutes and then heated to 80° C. for 16 hours. The reaction mixture was diluted with water, extracted 3× with EtOAc, and the combined organic layers were washed with brine, dried (Na₂SO₄), concentrated, and purified on silica gel (0-33% EtOAc/petroleum ether) to obtain the desired product (100 mg, 0.147 mmol, 43%). MS (ESI): mass calcd. for C₂₈H₃₄F₄N₆O₂Si 590.2; found 591.6 [M+H]⁺.

Step D. 2-(5-fluoro-2-pyridyl)-6,6-dimethyl-3-[6-(3,3,3-trifluoropropyl)-1H-pyrazolo[3,4-b]pyridin-4-yl]-4,7-dihydropyrazolo[5,1-c][1,4]oxazine. 2-(5-Fluoropyridin-2-yl)-6,6-dimethyl-3-(6-(3,3,3-trifluoropropyl)-1-((2-(trimethylsilyl)ethoxy)methyl)-1H-pyrazolo[3,4-b]pyridin-4-yl)-6,7-dihydro-4H-pyrazolo[5,1-c][1,4]oxazine (100 mg, 0.142 mmol), TFA (1.5 mL), and dichloromethane (3 mL) were stirred for 2 hours at r.t., and concentrated. The residue was taken up in 7M methanolic ammonia (2 mL) and the solution stirred at r.t. for 30 minutes, then purified by revere-phase prep HPLC (Welch Xtimate C18 150*25 mm*5 μm column (eluent: 43% to 73% (v/v) CH₃CN and H₂O with 0.225% HCOOH)) to afford the title compound (39.8 mg, 0.085 mmol, 60%). MS (ESI): mass calcd. for C₂₂H₂₀F₄N₆O 460.2; m/z found 461.1 [M+H]⁺. ¹H NMR (400 MHz, DMSO-d₆): δ 13.55 (s, 1H), 8.47 (d, J=4.6 Hz, 1H), 7.90-7.81 (m, 1H), 7.77-7.68 (m, 1H), 7.31 (d, J=1.2 Hz, 1H), 7.12 (d, J=4.6 Hz, 1H), 4.84 (s, 2H), 4.12 (s, 2H), 2.63-2.53 (m, 2H), 1.46-1.32 (m, 8H).

### Example 392: 3-(3-Chloro-5-fluoro-1H-pyrazolo[3,4-b]pyridin-4-yl)-2-(5-fluoropyridin-2-yl)-6,6-dimethyl-6,7-dihydro-4H-pyrazolo[5,1-c][1,4]oxazine

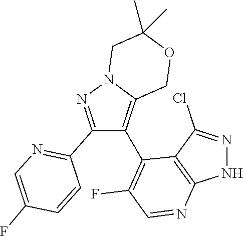 [SMILES: CC1(C)Cn2nc(-c3ccc(F)cn3)c(-c3c(F)cnc4[nH]nc(Cl)c34)c2CO1]

The title compound was prepared in a manner analogous to Example 224, except using 3-(5-fluoro-1H-pyrazolo[3,4-b]pyridin-4-yl)-2-(5-fluoropyridin-2-yl)-6,6-dimethyl-6,7-dihydro-4H-pyrazolo[5,1-c][1,4]oxazine (Example 389) instead of Example 9. MS (ESI): mass calcd. for C₁₉H₁₅ClF₂N₆O 416.1; m/z found 417.1 [M+H]⁺. ¹H NMR (400 MHz, CD₃OD): δ 8.52 (d, J=2.4 Hz, 1H), 8.03 (d, J=2.9 Hz, 1H), 7.93 (dd, J=8.9, 4.5 Hz, 1H), 7.56 (td, J=8.6, 2.9 Hz, 1H), 4.74 (d, J=3.2 Hz, 2H), 4.15 (s, 2H), 1.43 (d, J=1.8 Hz, 6H). N—H proton not observed.

### Example 393: 3-(3,6-Dimethyl-1H-pyrazolo[3,4-b]pyridin-4-yl)-2-(5-fluoro-6-methylpyridin-2-yl)-6,6-dimethyl-6,7-dihydro-4H-pyrazolo[5,1-c][1,4]oxazine

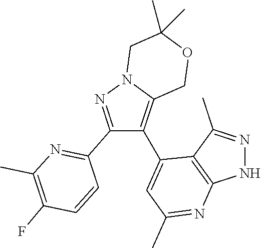 [SMILES: Cc1cc(-c2c(-c3ccc(F)c(C)n3)nn3c2COC(C)(C)C3)c2c(C)n[nH]c2n1]

To a vial containing 2-(5-fluoropyridin-2-yl)-6,6-dimethyl-3-(1H-pyrazolo[3,4-b]pyridin-4-yl)-6,7-dihydro-4H-pyrazolo[5,1-c][1,4]oxazine (Example 150, 36.4 mg, 0.1 mmol) and [4,4′-bis(1,1-dimethylethyl)-2,2′-bipyridine-N1,N1′]bis[3, 5-difluoro-2-[5-(trifluoromethyl)-2-pyridinyl-N]phenyl-C]Iridium(III) hexafluorophosphate, [Ir{dF(CF₃)ppy}₂(dtbpy)]PF₆ (1.1 mg, 0.001 mmol) was added MeCN (0.5 mL) and TFA (0.5 mL) followed by tert-butyl peroxyacetate (79 mg, 0.3 mmol). The reaction mixture was placed in a PennOC Photoreactor M1 (450 nm wavelength, 100% LED power, 100% fan power, and 750 rpm stirring). for 1 hour after which no change was observed by LCMS. Additional [4,4′-bis(1,1-dimethylethyl)-2,2′-bipyridine-N1,N1′]bis[3,5-difluoro-2-[5-(trifluoromethyl)-2-pyridinyl-N]phenyl-C]Iridium(III) hexafluorophosphate, [Ir{dF(CF₃)ppy}₂(dtbpy)]PF₆ (1.1 mg, 0.001 mmol) and tert-butyl peroxyacetate (79 mg, 0.3 mmol) was added, the vial was capped, and the reaction was sparged with N₂ for two minutes. The vial was then placed in a PennOC Photoreactor M1 (450 nm wavelength, 100% LED power, 100% fan power, and 750 rpm stirring) for 3 hours. The reaction mixture was diluted with EtOAc and saturated aq. Na₂CO₃ until neutral. The organic layer was separated, dried over MgSO₄, filtered, and concentrated to produce an orange solid. The orange solid was dissolved in DMF (1.5 mL) and purified via reverse phase prep HPLC (NH₄OH aq/MeCN, C18 column) to provide the title compound as a white solid (2 mg, 5%). MS (ESI): mass calcd. for C₂₂H₂₃FN₆O 406.2; m/z found 407.2 [M+H]⁺. ¹H NMR (600 MHz, MeOD) δ 7.41 (d, J=5.3 Hz, 1H), 7.25 (s, 1H), 7.06 (s, 1H), 4.99 (s, 2H), 4.15 (s, 2H), 2.69 (s, 3H), 2.27 (dd, J=1.8, 0.7 Hz, 3H), 2.17 (d, J=3.1 Hz, 3H), 1.48 (s, 6H).

### Example 394: 2-(4-Fluorophenyl)-6,6-dimethyl-3-(6-methyl-1H-pyrazolo[3,4-d]pyrimidin-4-yl)-4,7-dihydropyrazolo[5,1-c][1,4]oxazine

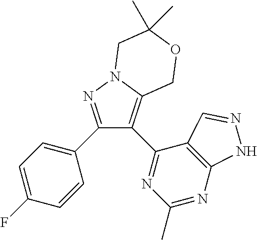 [SMILES: Cc1nc(-c2c(-c3ccc(F)cc3)nn3c2COC(C)(C)C3)c2cn[nH]c2n1]

Step A. 2-(4-Fluorophenyl)-6,6-dimethyl-3-(6-methyl-1-((2-(trimethylsilyl)ethoxy)methyl)-1H-pyrazolo[3,4-d]pyrimidin-4-yl)-6,7-dihydro-4H-pyrazolo[5,1-c][1,4]oxazine. 2-(4-Fluorophenyl)-6,6-dimethyl-3-(4,4,5,5-tetramethyl-1,3,2-dioxaborolan-2-yl)-6,7-dihydro-4H-pyrazolo[5,1-c][1,4]oxazine (Intermediate 228, 566 mg, 1.22 mmol), 4-chloro-6-methyl-1-((2-(trimethylsilyl)ethoxy)methyl)-1H-pyrazolo[3,4-d]pyrimidine (Intermediate 212, 400 mg, 1.34 mmol), K₃PO₄ (777 mg, 3.66 mmol), 1,4-dioxane (8 mL), and H₂O (2 mL) were combined. The resultant mixture was sparged with N₂ for 5 minutes and then treated with Pd(dtbpf)Cl₂ (82 mg, 0.13 mmol). The resultant mixture was sparged with N₂ for another 5 minutes and then heated at 90° C. via microwave irradiation for 1 hour. The reaction mixture was cooled to room-temperature. The reaction mixture was poured into water (20 mL) and extracted with ethyl acetate (20 mL×3). The combined organic extracts were washed with brine (10 mL), dried over anhydrous Na₂SO₄, filtered, and concentrated to dryness under reduced pressure. The resulting residue was purified by FCC (SiO₂, eluent: petroleum ether:ethyl acetate=1:0 to 3:1) to afford the title compound (450 mg, 72%) as a yellow solid. MS (ESI): mass calcd. for C₂₆H₃₃FN₆O₂Si 508.2; m/z found 509.7 [M+H]⁺.

Step B. 4-(6,6-Difluoro-2-(4-fluorophenyl)-4,5,6,7-tetrahydropyrazolo[1,5-a]pyridin-3-yl)-6-methyl-1H-pyrazolo[3,4-d]pyrimidine. 2-(4-Fluorophenyl)-6,6-dimethyl-3-(6-methyl-1-((2-(trimethylsilyl)ethoxy)methyl)-1H-pyrazolo[3,4-d]pyrimidin-4-yl)-6,7-dihydro-4H-pyrazolo[5,1-c][1,4]oxazine (450 mg, 0.723 mmol), TFA (5 mL), and dichloromethane (3 mL) were added to a 50 mL round-bottomed flask. The resultant solution was stirred at room-temperature for 16 hours. The reaction solution was concentrated to dryness under reduced pressure to afford the title compound, which was diluted with methanol (5 mL), and then treated with ammonia 7 M in methanol (2 mL). The resultant solution was stirred at room-temperature for 30 min. The reaction mixture was concentrated to dryness under reduced pressure to afford the title compound. The product was poured into water (20 mL) and extracted with dichloromethane:methanol=10:1 (20 mL×3). The combined organic extracts were washed with brine (50 mL), dried over anhydrous Na₂SO₄, filtered, and concentrated to dryness under reduced pressure. The resulting residue was triturated with ethyl acetate (20 mL) and the suspension isolated via filtration. The filter cake was washed with ethyl acetate (20 mL) before drying under reduced pressure to afford the title compound (208.7 mg, 76%) as a white solid. MS (ESI): mass calcd. for C₂₀H₁₉FN₆O 378.2; m/z found 379.1 [M+H]⁺. ¹H NMR (400 MHz, DMSO-d₆): δ 13.62 (s, 1H), 7.51-7.38 (m, 2H), 7.26-7.14 (m, 2H), 6.75-6.63 (m, 1H), 5.14-5.00 (m, 2H), 4.16-4.06 (m, 2H), 2.74-2.63 (m, 3H), 1.36 (s, 6H).

### Example 395: N-(4-(2-(5-Fluoropyridin-2-yl)-6,6-bis(methyl-d₃)-6,7-dihydro-4H-pyrazolo[5,1-c][1,4]oxazin-3-yl)pyridin-2-yl)propionamide

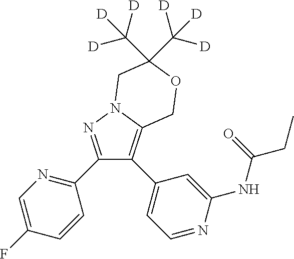 [SMILES: [2H]C([2H])([2H])C1(C([2H])([2H])[2H])Cn2nc(-c3ccc(F)cn3)c(-c3ccnc(NC(=O)CC)c3)c2CO1]

A solution of (2-propionamidopyridin-4-yl)boronic acid (58.4 mg, 0.3 mmol), 3-bromo-2-(5-fluoropyridin-2-yl)-6,6-bis(methyl-d₃)-6,7-dihydro-4H-pyrazolo[5,1-c][1,4]oxazine (Intermediate 86, 50 mg, 0.15 mmol), Cs₂CO₃ (197 mg, 0.6 mmol), 2-methyl-2-butanol (2 mL), and H₂O (0.5 mL) was sparged with N₂ for 5 minutes and then treated with CataCXium® A-Pd-G3 (22 mg, 0.03 mmol). The reaction mixture was sparged with N₂ for another 5 minutes and then heated at 80° C. for 18 hours. The reaction mixture was cooled to room-temperature. The suspension was filtered through a pad of Celite® and the pad washed with ethyl acetate (10 mL). The filtrate was diluted with H₂O (5 mL) and the resultant mixture extracted with ethyl acetate (10 mL×3). The combined organic extracts were dried over anhydrous Na₂SO₄, filtered, and concentrated to dryness under reduced pressure. The resulting residue was purified by prep-HPLC over Boston Green ODS 150*30 mm*5 um (eluent: 30% to 60% (v/v) CH₃CN and H₂O with 0.225% HCOOH). The pure fractions were collected and the volatiles were removed under reduced pressure. The product was suspended in water (20 mL), the mixture frozen using dry ice/acetone, and then lyophilized to dryness to afford the title compound as a white solid (33 mg, 27%). MS (ESI): mass calcd. for C₂₁H₁₆D₆FN₅O₂ 401.2; m/z found 402.1 [M+H]⁺. ¹H NMR (400 MHz, CDCl₃) δ 8.44 (d, J=2.9 Hz, 1H), 8.13 (d, J=4.6 Hz, 2H), 8.03 (s, 1H), 7.58 (m, 1H), 7.39 (m, 1H), 6.83-6.73 (m, 1H), 4.98 (s, 2H), 4.06 (s, 2H), 2.44 (q, J=7.5 Hz, 2H), 1.25 (t, J=7.6 Hz, 3H).

### Example 396: 2-(5-Fluoropyridin-2-yl)-6,6-bis(methyl-d₃)-3-(pyrazolo[1,5-a]pyridin-5-yl)-6,7-dihydro-4H-pyrazolo[5,1-c][1,4]oxazine

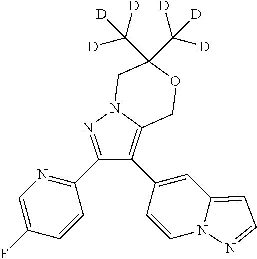 [SMILES: [2H]C([2H])([2H])C1(C([2H])([2H])[2H])Cn2nc(-c3ccc(F)cn3)c(-c3ccn4nccc4c3)c2CO1]

A solution of 5-(4,4,5,5-tetramethyl-1,3,2-dioxaborolan-2-yl)pyrazolo[1,5-a]pyridine (Intermediate 224) (57.3 mg, 0.235 mmol), 3-bromo-2-(5-fluoropyridin-2-yl)-6,6-bis(methyl-d₃)-6,7-dihydro-4H-pyrazolo[5,1-c][1,4]oxazine (Intermediate 86, 65 mg, 0.20 mmol), Cs₂CO₃ (191 mg, 0.587 mmol), 2-methyl-2-butanol (6 mL), and H₂O (1.5 mL) was sparged with N₂ for 5 minutes and then treated with CataCXium® A-Pd-G3 (28.5 mg, 0.039 mmol). The mixture was sparged with N₂ for another 5 minutes and then heated at 100° C. via microwave irradiation for 1 hour. The reaction mixture was cooled to room-temperature. The suspension was filtered through a pad of Celite® and the pad washed with ethyl acetate (50 mL). The combined organics were washed with brine (50 mL), dried over anhydrous Na₂SO₄, filtered, and concentrated to dryness under reduced pressure. The resulting residue was purified by prep-HPLC using a Boston Prime C18 (150 mm×30 mm×Sum) ((eluent: 35% to 65% (v/v) CH₃CN and H₂O with 0.05% NH₃H₂O) to afford pure product. The product was suspended in water (10 mL), the mixture frozen using dry ice/acetone, and then lyophilized to dryness to afford the title compound as a white solid (29.5 mg, 40%). MS (ESI): mass calcd. for C₂₀H₁₂D₆FN₅O 369.2; m/z found 370.2 [M+H]⁺. ¹H NMR (400 MHz, CDCl₃) δ 8.44 (d, J=2.8 Hz, 1H), 8.40 (d, J=7.0 Hz, 1H), 7.97 (d, J=2.3 Hz, 1H), 7.59-7.54 (m, 1H), 7.40-7.33 (m, 2H), 6.55 (dd, J=1.8, 7.3 Hz, 1H), 6.50 (d, J=1.8 Hz, 1H), 4.92 (s, 2H), 4.09 (s, 2H).

### Example 397: 3-(5-Fluoro-1H-pyrrolo[2,3-b]pyridin-4-yl)-2-(5-fluoropyridin-2-yl)-6,6-bis(methyl-d₃)-6,7-dihydro-4H-pyrazolo[5,1-c][1,4]oxazine

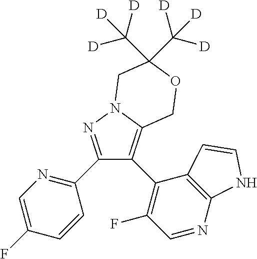 [SMILES: [2H]C([2H])([2H])C1(C([2H])([2H])[2H])Cn2nc(-c3ccc(F)cn3)c(-c3c(F)cnc4[nH]ccc34)c2CO1]

The title compound was prepared in a manner analogous to Example 1, Steps A-B, except using lithium 2-(2-(5-fluoropyridin-2-yl)-6,6-bis(methyl-d₃)-6,7-dihydro-4H-pyrazolo[5,1-c][1,4]oxazin-3-yl)-2-hydroxy-4,4,5,5-tetramethyl-1,3,2-dioxaborolan-2-uide (Intermediate 230) instead of 3-bromo-2-(5-fluoropyridin-2-yl)-6,7-dihydro-4H-pyrazolo[5,1-c][1,4]oxazine (Intermediate 37) and 4-bromo-5-fluoro-1-((2-(trimethylsilyl)ethoxy)methyl)-1H-pyrrolo[2,3-b]pyridine (Intermediate 210) instead of 4-(4,4,5,5-tetramethyl-1,3,2-dioxaborolan-2-yl)-1-((2-(trimethylsilyl)ethoxy)methyl)-1H-pyrrolo[2,3-b]pyridine (Intermediate 21). MS (ESI): mass calcd. for C₂₀H₁₁D₆F₂N₅O 387.2; m/z found 388.3 [M+H]⁺. ¹H NMR (400 MHz, DMSO-d₆): δ 11.75 (br s, 1H), 8.22 (d, J=2.9 Hz, 1H), 8.15 (d, J=2.6 Hz, 1H), 7.90-7.85 (m, 1H), 7.75-7.68 (m, 1H), 7.46-7.44 (m, 1H), 6.00-5.95 (m, 1H), 4.74-4.56 (m, 2H), 4.19-4.08 (m, 2H).

### Example 398: 3-(3-Fluoro-1H-pyrazolo[3,4-b]pyridin-4-yl)-2-(5-fluoropyridin-2-yl)-6,6-bis(methyl-d₃)-6,7-dihydro-4H-pyrazolo[5,1-c][1,4]oxazine

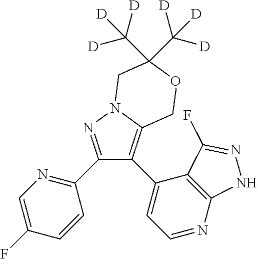 [SMILES: [2H]C([2H])([2H])C1(C([2H])([2H])[2H])Cn2nc(-c3ccc(F)cn3)c(-c3ccnc4[nH]nc(F)c34)c2CO1]

The title compound was prepared in a manner analogous to Example 1, Steps A-B, except using lithium 2-(2-(5-fluoropyridin-2-yl)-6,6-bis(methyl-d₃)-6,7-dihydro-4H-pyrazolo[5,1-c][1,4]oxazin-3-yl)-2-hydroxy-4,4,5,5-tetramethyl-1,3,2-dioxaborolan-2-uide (Intermediate 230) instead of 3-bromo-2-(5-fluoropyridin-2-yl)-6,7-dihydro-4H-pyrazolo[5,1-c][1,4]oxazine (Intermediate 37) and 4-bromo-3-fluoro-1H-pyrazolo[3,4-b]pyridine (Intermediate 148) instead of 4-(4,4,5,5-tetramethyl-1,3,2-dioxaborolan-2-yl)-1-((2-(trimethylsilyl)ethoxy)methyl)-1H-pyrrolo[2,3-b]pyridine (Intermediate 21) in Step A. MS (ESI): mass calcd. for C₁₉H₁₀D₆F₂N₆O 388.2; m/z found 389.3 [M+H]⁺. ¹H NMR (400 MHz, CD₃OD): δ 8.52 (d, J=4.8 Hz, 1H), 8.11 (d, J=2.9 Hz, 1H), 7.95-7.81 (m, 1H), 7.60 (td, J=8.6, 2.9 Hz, 1H), 7.13 (d, J=4.8 Hz, 1H), 5.05 (s, 1H), 4.81-4.72 (m, 1H), 4.14 (d, J=2.0 Hz, 2H). N—H proton is not observed.

### Example 399: 3-(5-Fluoro-1H-pyrazolo[3,4-b]pyridin-4-yl)-2-(5-fluoropyridin-2-yl)-6,6-bis(methyl-d₃)-6,7-dihydro-4H-pyrazolo[5,1-c][1,4]oxazine

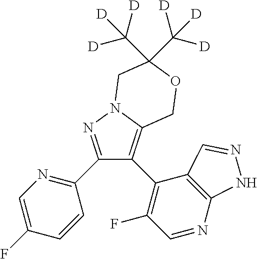 [SMILES: [2H]C([2H])([2H])C1(C([2H])([2H])[2H])Cn2nc(-c3ccc(F)cn3)c(-c3c(F)cnc4[nH]ncc34)c2CO1]

The title compound was prepared in a manner analogous to Example 1, Steps A-B, except using lithium 2-(2-(5-fluoropyridin-2-yl)-6,6-bis(methyl-d₃)-6,7-dihydro-4H-pyrazolo[5,1-c][1,4]oxazin-3-yl)-2-hydroxy-4,4,5,5-tetramethyl-1,3,2-dioxaborolan-2-uide (Intermediate 230) instead of 3-bromo-2-(5-fluoropyridin-2-yl)-6,7-dihydro-4H-pyrazolo[5,1-c][1,4]oxazine (Intermediate 37) and 5-fluoro-4-iodo-1-(tetrahydro-2H-pyran-2-yl)-1H-pyrazolo[3,4-b]pyridine (Intermediate 213) instead of 4-(4,4,5,5-tetramethyl-1,3,2-dioxaborolan-2-yl)-1-((2-(trimethylsilyl)ethoxy)methyl)-1H-pyrrolo[2,3-b]pyridine (Intermediate 21) in Step A. MS (ESI): mass calcd. for C₁₉H₁₀D₆F₂N₆O 388.2; m/z found 389.1 [M+H]⁺. ¹H NMR (400 MHz, CD₃OD) δ 8.47 (d, J=2.8 Hz, 1H), 8.11 (dt, J=2.9, 0.7 Hz, 1H), 7.86 (ddd, J=8.9, 4.5, 0.6 Hz, 1H), 7.59 (td, J=8.6, 2.9 Hz, 1H), 7.46 (s, 1H), 4.78 (s, 2H), 4.15 (d, J=2.8 Hz, 2H).

### Example 400: 3-(3-Chloro-1H-pyrazolo[3,4-b]pyridin-4-yl)-2-(5-fluoropyridin-2-yl)-6,6-bis(methyl-d₃)-6,7-dihydro-4H-pyrazolo[5,1-c][1,4]oxazine

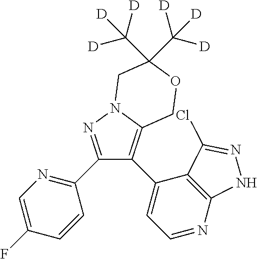 [SMILES: [2H]C([2H])([2H])C1(C([2H])([2H])[2H])Cn2nc(-c3ccc(F)cn3)c(-c3ccnc4[nH]nc(Cl)c34)c2CO1]

The title compound was prepared in a manner analogous to Example 224, except using 2-(5-fluoropyridin-2-yl)-6,6-bis(methyl-d₃)-3-(1H-pyrazolo[3,4-b]pyridin-4-yl)-6,7-dihydro-4H-pyrazolo[5,1-c][1,4]oxazine (Example 142) instead of Example 9. After purification by acidic HPLC the concentrated material was dissolved in EtOAc and washed with sat'd NaHCO₃ sol'n (×3). The organics were dried with sodium sulfate, filtered, and concentrated. MS (ESI): mass calcd. for C₁₉H₁₀D₆ClFN₆O, 404.1; m/z found, 405.2 [M+H]⁺. ¹H NMR (400 MHz, CD₃OD): δ 8.53 (d, J=4.7 Hz, 1H), 8.08-8.01 (m, 1H), 7.79 (ddd, J=8.9, 4.5, 0.6 Hz, 1H), 7.51 (td, J=8.6, 2.9 Hz, 1H), 7.12 (d, J=4.7 Hz, 1H), 4.91-4.87 (m, 1H), 4.68 (d, J=15.9 Hz, 1H), 4.12 (d, J=1.4 Hz, 2H). N—H proton not observed.

### Example 401: 2-(5-Fluoropyridin-2-yl)-6,6-bis(methyl-d₃)-3-(6-methyl-1H-pyrazolo[3,4-b]pyridin-4-yl)-6,7-dihydro-4H-pyrazolo[5,1-c][1,4]oxazine

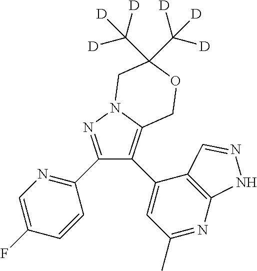 [SMILES: [2H]C([2H])([2H])C1(C([2H])([2H])[2H])Cn2nc(-c3ccc(F)cn3)c(-c3cc(C)nc4[nH]ncc34)c2CO1]

The title compound was prepared in a manner analogous to Example 1, Steps A-B, except using 3-bromo-2-(5-fluoropyridin-2-yl)-6,6-bis(methyl-d₃)-6,7-dihydro-4H-pyrazolo[5,1-c][1,4]oxazine (Intermediate 86) instead of 3-bromo-2-(5-fluoropyridin-2-yl)-6,7-dihydro-4H-pyrazolo[5,1-c][1,4]oxazine (Intermediate 37) and 1-(4-methoxybenzyl)-6-methyl-4-(4,4,5,5-tetramethyl-1,3,2-dioxaborolan-2-yl)-1H-pyrazolo[3,4-b]pyridine (Intermediate 217) instead of 4-(4,4,5,5-tetramethyl-1,3,2-dioxaborolan-2-yl)-1-((2-(trimethylsilyl)ethoxy)methyl)-1H-pyrrolo[2,3-b]pyridine (Intermediate 21) in Step A. MS (ESI): mass calcd. for C₂₀H₁₃D₆FN₆O 384.2; m/z found 385.2 [M+H]⁺. ¹H NMR (400 MHz, CD₃OD) δ 8.20 (dt, J=3.0, 0.7 Hz, 1H), 7.78-7.65 (m, 1H), 7.58 (td, J=8.6, 2.9 Hz, 1H), 7.24 (s, 1H), 7.02 (s, 1H), 4.93 (s, 2H), 4.12 (s, 2H), 2.65 (s, 3H).

### Example 402: 3-(6-(Difluoromethyl)-1H-pyrazolo[3,4-b]pyridin-4-yl)-2-(5-fluoropyridin-2-yl)-6,6-bis(methyl-d₃)-6,7-dihydro-4H-pyrazolo[5,1-c][1,4]oxazine

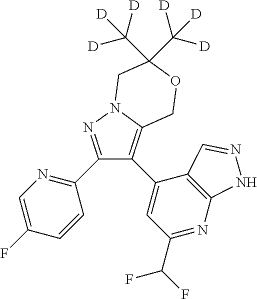 [SMILES: [2H]C([2H])([2H])C1(C([2H])([2H])[2H])Cn2nc(-c3ccc(F)cn3)c(-c3cc(C(F)F)nc4[nH]ncc34)c2CO1]

The title compound was prepared in a manner analogous to Example 1, Steps A-B, except using 3-bromo-2-(5-fluoropyridin-2-yl)-6,6-bis(methyl-d₃)-6,7-dihydro-4H-pyrazolo[5,1-c][1,4]oxazine (Intermediate 86) instead of 3-bromo-2-(5-fluoropyridin-2-yl)-6,7-dihydro-4H-pyrazolo[5,1-c][1,4]oxazine (Intermediate 37) and 6-(difluoromethyl)-4-(4,4,5,5-tetramethyl-1,3,2-dioxaborolan-2-yl)-1-((2-(trimethylsilyl)ethoxy)methyl)-1H-pyrazolo[3,4-b]pyridine (Intermediate 221) instead of 4-(4,4,5,5-tetramethyl-1,3,2-dioxaborolan-2-yl)-1-((2-(trimethylsilyl)ethoxy)methyl)-1H-pyrrolo[2,3-b]pyridine (Intermediate 21) in Step A. Purification via preparative HPLC using a Phenomenex Gemini NX-C18 75×30 mm×3 μm column (eluent: 30% to 60% (v/v) CH₃CN and H₂O with 0.05% NH₃H₂O+10 mM NH₄HCO³) was employed for Step B. MS (ESI): mass calcd. for C₂₀H₁₁D₆F₃N₆O 420.2; m/z found 421.1 [M+H]⁺. ¹H NMR (400 MHz, DMSO-d₆): δ 13.82 (br s, 1H), 8.19 (d, J=2.8 Hz, 1H), 7.93 (dd, J=4.5, 8.8 Hz, 1H), 7.79 (dt, J=3.0, 8.8 Hz, 1H), 7.42 (s, 1H), 7.37 (s, 1H), 7.04 (t, J=54.8 Hz, 1H), 4.88 (s, 2H), 4.13 (s, 2H).

### Example 403: 3-(3-Chloro-5-fluoro-1H-pyrazolo[3,4-b]pyridin-4-yl)-2-(5-fluoropyridin-2-yl)-6,6-bis(methyl-d₃)-6,7-dihydro-4H-pyrazolo[5,1-c][1,4]oxazine

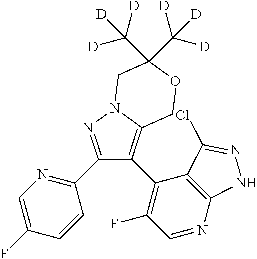 [SMILES: [2H]C([2H])([2H])C1(C([2H])([2H])[2H])Cn2nc(-c3ccc(F)cn3)c(-c3c(F)cnc4[nH]nc(Cl)c34)c2CO1]

The title compound was prepared in a manner analogous to Example 224, except using 3-(5-fluoro-1H-pyrazolo[3,4-b]pyridin-4-yl)-2-(5-fluoropyridin-2-yl)-6,6-bis(methyl-d₃)-6,7-dihydro-4H-pyrazolo[5,1-c][1,4]oxazine (Example 399) instead of Example 9. An additional basic purification (basic ACCQ-prep. HPLC (20 mM NH₄OH in H₂O and neutral CH₃CN) was carried out before acidic HPLC. MS (ESI): mass calcd. for C₁₉H₉D₆ClF₂N₆O 422.1; m/z found 423.2 [M+H]⁺. ¹H NMR (400 MHz, CD₃OD): δ 8.52 (d, J=2.4 Hz, 1H), 8.02 (d, J=2.9 Hz, 1H), 7.93 (dd, J=8.9, 4.5 Hz, 1H), 7.56 (td, J=8.7, 3.0 Hz, 1H), 4.74 (d, J=3.3 Hz, 2H), 4.14 (d, J=0.8 Hz, 2H). N—H proton not observed.

### Example 404: 3-(5-Fluoro-3-methyl-1H-pyrazolo[3,4-b]pyridin-4-yl)-2-(5-fluoropyridin-2-yl)-6,6-bis(methyl-d₃)-6,7-dihydro-4H-pyrazolo[5,1-c][1,4]oxazine

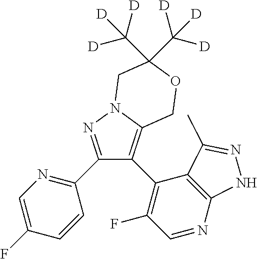 [SMILES: [2H]C([2H])([2H])C1(C([2H])([2H])[2H])Cn2nc(-c3ccc(F)cn3)c(-c3c(F)cnc4[nH]nc(C)c34)c2CO1]

Step A: 3-(5-Fluoro-3-methyl-1-(tetrahydro-2H-pyran-2-yl)-1H-pyrazolo[3,4-b]pyridin-4-yl)-2-(5-fluoropyridin-2-yl)-6,6-bis(methyl-d₃)-6,7-dihydro-4H-pyrazolo[5,1-c][1,4]oxazine.

Cs₂CO₃ (235 mg, 0.721 mmol) was added to a mixture of 3-bromo-2-(5-fluoropyridin-2-yl)-6,6-bis(methyl-d₃)-6,7-dihydro-4H-pyrazolo[5,1-c][1,4]oxazine (Intermediate 86, 80 mg, 0.24 mmol), 5-fluoro-3-methyl-1-(tetrahydro-2H-pyran-2-yl)-4-(4,4,5,5-tetramethyl-1,3,2-dioxaborolan-2-yl)-1H-pyrazolo[3,4-b]pyridine (Intermediate 206, 174 mg, 0.482 mmol), 2-methyl-2-butanol (4 mL) and H₂O (0.8 mL). The mixture was sparged with Ar for 5 minutes and then treated with CataCXium® A-Pd-G3 (18 mg, 0.025 mmol). The mixture was sparged with Ar for another 5 minutes and then stirred and heated at 90° C. for 2 h via microwave irradiation. The reaction mixture was cooled to RT. The mixture was quenched with water (30 mL), extracted with EtOAc (50 mL×3). The extracts were dried over anhydrous Na₂SO₄, filtered and concentrated to dryness under reduced pressure. The resulting residue was purified by FCC (eluent: EtOAc:pet ether=1:10 to 1:3) to afford the title compound (25 mg, 20%) as yellow solid. MS (ESI): mass calcd. for C₂₀H₁₂D₆F₂N₆O 486.3; m/z found 487.2 [M+H]⁺. Step B: 3-(5-Fluoro-3-methyl-1H-pyrazolo[3,4-b]pyridin-4-yl)-2-(5-fluoropyridin-2-yl)-6,6-bis(methyl-d₃)-6,7-dihydro-4H-pyrazolo[5,1-c][1,4]oxazine. 4M HCl/dioxane (0.2 mL) were added to a mixture of 3-(5-fluoro-3-methyl-1-(tetrahydro-2H-pyran-2-yl)-1H-pyrazolo[3,4-b]pyridin-4-yl)-2-(5-fluoropyridin-2-yl)-6,6-bis(methyl-d₃)-6,7-dihydro-4H-pyrazolo[5,1-c][1,4]oxazine (20 mg, 0.041 mmol) and tetrahydrofuran (3 mL). The reaction mixture was stirred at r.t. for 2 h. The mixture was concentrated to dryness which was subjected to preparative HPLC using a Boston Prime C18 150×30 mm×5 μm (eluent: 25% to 55% (v/v) CH₃CN and H₂O with 0.05% NH₃) to afford the pure product. The product was suspended in water (10 mL), then lyophilized to dryness to afford the title compound (11.7 mg, 70%) as a white solid. MS (ESI): mass calcd. for C₂₀H₁₂D₆F₂N₆O 402.2; m/z found 403.1 [M+H]⁺. ¹H NMR (400 MHz, DMSO-d₆) δ 13.29 (br s, 1H), 8.48 (d, J=2.2 Hz, 1H), 8.08 (d, J=2.9 Hz, 1H), 7.94 (dd, J=4.6, 8.8 Hz, 1H), 7.73-7.66 (m, 1H), 4.66-4.51 (m, 2H), 4.17-4.07 (m, 2H), 1.86 (s, 3H).

### Example 405: 3-(3-Chloro-6-methyl-1H-pyrazolo[3,4-b]pyridin-4-yl)-2-(5-fluoropyridin-2-yl)-6,6-bis(methyl-d₃)-6,7-dihydro-4H-pyrazolo[5,1-c][1,4]oxazine

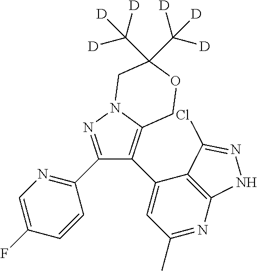 [SMILES: [2H]C([2H])([2H])C1(C([2H])([2H])[2H])Cn2nc(-c3ccc(F)cn3)c(-c3cc(C)nc4[nH]nc(Cl)c34)c2CO1]

The title compound was prepared in a manner analogous to Example 224, except using 2-(5-fluoropyridin-2-yl)-6,6-bis(methyl-d₃)-3-(6-methyl-1H-pyrazolo[3,4-b]pyridin-4-yl)-6,7-dihydro-4H-pyrazolo[5,1-c][1,4]oxazine (Example 401) instead of 5-fluoro-4-(2-(5-fluoropyridin-2-yl)-5, 6-dihydro-4H-pyrrolo[1,2-b]pyrazol-3-yl)-6-methyl-1H-pyrazolo[3,4-b]pyridine (Example 9). MS (ESI): mass calcd. for C₂₀H₁₂D₆ClFN₆O, 418.2; m/z found, 419.1 [M+H]⁺. ¹H NMR (400 MHz, CD₃OD): δ 8.07 (d, J=2.9 Hz, 1H), 7.87-7.67 (m, 1H), 7.51 (td, J=8.6, 3.0 Hz, 1H), 7.02 (s, 1H), 4.87 (d, J=15.9 Hz, 1H), 4.66 (d, J=15.9 Hz, 1H), 4.12 (d, J=1.2 Hz, 2H), 2.64 (s, 3H). N—H proton not observed.

### Example 406: 3-(5-Fluoro-6-methyl-1H-pyrazolo[3,4-b]pyridin-4-yl)-2-(5-fluoropyridin-2-yl)-6,6-bis(methyl-d₃)-6,7-dihydro-4H-pyrazolo[5,1-c][1,4]oxazine

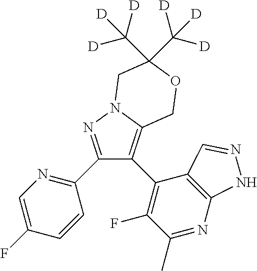 [SMILES: [2H]C([2H])([2H])C1(C([2H])([2H])[2H])Cn2nc(-c3ccc(F)cn3)c(-c3c(F)c(C)nc4[nH]ncc34)c2CO1]

The title compound was prepared in a manner analogous to Example 1, Steps A-B, except using 5-fluoro-4-iodo-6-methyl-1-(tetrahydro-2H-pyran-2-yl)-1H-pyrazolo[3,4-b]pyridine. (Intermediate 215) instead of 3-bromo-2-(5-fluoropyridin-2-yl)-6,7-dihydro-4H-pyrazolo[5,1-c][1,4]oxazine (Intermediate 37), lithium 2-(2-(5-fluoropyridin-2-yl)-6,6-bis(methyl-d₃)-6,7-dihydro-4H-pyrazolo[5,1-c][1,4]oxazin-3-yl)-2-hydroxy-4,4,5,5-tetramethyl-1,3,2-dioxaborolan-2-uide (Intermediate 230) instead of 4-(4,4,5,5-tetramethyl-1,3,2-dioxaborolan-2-yl)-1-((2-(trimethylsilyl)ethoxy)methyl)-1H-pyrrolo[2,3-b]pyridine (Intermediate 21), CataCXium® A Pd G4 instead of CataCXium® A Pd G3, and THF instead of 2-methyl-2-butanol in Step A. MS (ESI): mass calcd. for C₂₀H₁₂D₆F₂N₆O 402.2; m/z found 403.1 [M+H]⁺. ¹H NMR (400 MHz, DMSO-d₆) δ 13.52 (br s, 1H), 8.20 (d, J=2.8 Hz, 1H), 7.98-7.91 (m, 1H), 7.81-7.73 (m, 1H), 7.49 (s, 1H), 4.80-4.65 (m, 2H), 4.15 (s, 2H), 2.54 (d, J=3.5 Hz, 3H).

### Example 407: 3-(5-Chloro-6-methyl-1H-pyrazolo[3,4-b]pyridin-4-yl)-2-(5-fluoropyridin-2-yl)-6,6-bis(methyl-d₃)-6,7-dihydro-4H-pyrazolo[5,1-c][1,4]oxazine

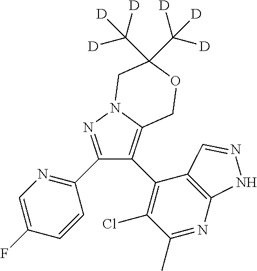 [SMILES: [2H]C([2H])([2H])C1(C([2H])([2H])[2H])Cn2nc(-c3ccc(F)cn3)c(-c3c(Cl)c(C)nc4[nH]ncc34)c2CO1]

The title compound was prepared in a manner analogous to Example 337, using 2-(5-fluoropyridin-2-yl)-6,6-bis(methyl-d₃)-3-(6-methyl-1H-pyrazolo[3,4-b]pyridin-4-yl)-6,7-dihydro-4H-pyrazolo[5,1-c][1,4]oxazine (Example 401) instead of Example 328. MS (ESI): mass calcd. for C₂₀H₁₂ClD₆FN₆O 418.2; m/z found 419.1 [M+H]⁺. ¹H NMR (400 MHz, DMSO-d₆): δ 13.62 (s, 1H), 8.15 (d, J=2.8 Hz, 1H), 7.95-7.90 (m, 1H), 7.76-7.69 (m, 1H), 7.58 (s, 1H), 4.68-4.55 (m, 2H), 4.21-4.10 (m, 2H), 2.67 (s, 3H).

### Example 408: 3-(3-Chloro-5-fluoro-6-methyl-1H-pyrazolo[3,4-b]pyridin-4-yl)-2-(5-fluoropyridin-2-yl)-6,6-bis(methyl-d₃)-6,7-dihydro-4H-pyrazolo[5,1-c][1,4]oxazine

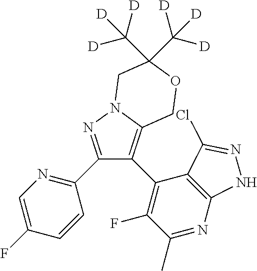 [SMILES: [2H]C([2H])([2H])C1(C([2H])([2H])[2H])Cn2nc(-c3ccc(F)cn3)c(-c3c(F)c(C)nc4[nH]nc(Cl)c34)c2CO1]

The title compound was prepared in a manner analogous to Example 224, except using 3-(5-fluoro-6-methyl-1H-pyrazolo[3,4-b]pyridin-4-yl)-2-(5-fluoropyridin-2-yl)-6,6-bis(methyl-d₃)-6,7-dihydro-4H-pyrazolo[5,1-c][1,4]oxazine (Example 406) instead of 5-fluoro-4-(2-(5-fluoropyridin-2-yl)-5,6-dihydro-4H-pyrrolo[1,2-b]pyrazol-3-yl)-6-methyl-1H-pyrazolo[3,4-b]pyridine (Example 9). MS (ESI): mass calcd. for C₂₀H₁₁D₆ClF₂N₆O, 436.1; m/z found, 437.2 [M+H]⁺. ¹H NMR (400 MHz, CD₃OD): δ 8.05 (d, J=2.8 Hz, 1H), 7.95-7.86 (m, 1H), 7.56 (td, J=8.7, 2.9 Hz, 1H), 4.79-4.65 (m, 2H), 4.14 (s, 2H), 2.61 (d, J=3.5 Hz, 3H). N—H proton not observed.

### Example 409: 3-(5-Fluoro-3,6-dimethyl-1H-pyrazolo[3,4-b]pyridin-4-yl)-2-(5-fluoropyridin-2-yl)-6,6-bis(methyl-d₃)-6,7-dihydro-4H-pyrazolo[5,1-c][1,4]oxazine

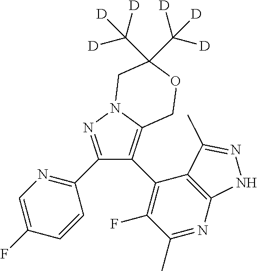 [SMILES: [2H]C([2H])([2H])C1(C([2H])([2H])[2H])Cn2nc(-c3ccc(F)cn3)c(-c3c(F)c(C)nc4[nH]nc(C)c34)c2CO1]

The title compound was prepared in a manner analogous to Example 430, Steps A-B, except using 3-bromo-2-(5-fluoropyridin-2-yl)-6,6-bis(methyl-d₃)-6,7-dihydro-4H-pyrazolo[5,1-c][1,4]oxazine (Intermediate 86) instead of 3-bromo-2-(5-fluoropyridin-2-yl)-6-(methyl-d₃)-6-(trifluoromethyl)-6,7-dihydro-4H-pyrazolo[5,1-c][1,4]oxazine (Intermediate 189) and heating at 90° C. via microwave irradiation for 1 hour instead of conventional heating in Step A. Purification via preparative HPLC using a Xtimate C18 150×40 mm×5 μm column (eluent: 30% to 60% (v/v) CH₃CN and H₂O with 0.05% NH₃H₂O) in Step B. MS (ESI): mass calcd. for C₂₁H₁₄D₆F₂N₆O 416.2; m/z found 417.1 [M+H]⁺. ¹H NMR (400 MHz, DMSO-d₆): δ 13.09 (br s, 1H), 8.13 (d, J=3.0 Hz, 1H), 7.99-7.93 (m, 1H), 7.76-7.68 (m, 1H), 4.61 (q, J=16.1 Hz, 2H), 4.15 (s, 2H), 2.54 (d, J=3.3 Hz, 3H), 1.85 (s, 3H).

### Example 410: 2-(4-Fluorophenyl)-6,6-bis(methyl-d₃)-3-(6-methyl-1H-pyrazolo[3,4-d]pyrimidin-4-yl)-6,7-dihydro-4H-pyrazolo[5,1-c][1,4]oxazine

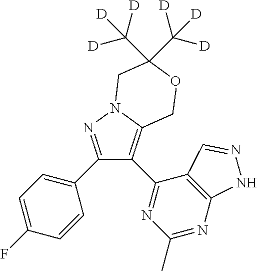 [SMILES: [2H]C([2H])([2H])C1(C([2H])([2H])[2H])Cn2nc(-c3ccc(F)cc3)c(-c3nc(C)nc4[nH]ncc34)c2CO1]

The title compound was prepared in a manner analogous to Example 236 Steps B-C, using 2-(4-fluorophenyl)-6,6-bis(methyl-d₃)-3-(4,4,5,5-tetramethyl-1,3,2-dioxaborolan-2-yl)-6,7-dihydro-4H-pyrazolo[5,1-c][1,4]oxazine instead of fluoropyridin-2-yl)-5,5-dimethyl-5,6-dihydro-4H-pyrrolo[1,2-b]pyrazol-3-yl)boronic acid (Example 236 product from Step A) and 4-chloro-6-methyl-1-((2-(trimethylsilyl)ethoxy)methyl)-1H-pyrazolo[3,4-d]pyrimidine (Intermediate 185) instead of 5-fluoro-4-iodo-1-((2-(trimethylsilyl)ethoxy)methyl)-1H-pyrazolo[3,4-b]pyridine (Intermediate 204) in Step B. MS (ESI): mass calcd. for C₂₀H₁₃D₆FN₆O 384.2; m/z found 385.1 [M+H]⁺. ¹H NMR (400 MHz, DMSO-d₆): 13.63 (br s, 1H), 7.53-7.37 (m, 2H), 7.28-7.10 (m, 2H), 6.69 (s, 1H), 5.06 (s, 2H), 4.09 (s, 2H), 2.68 (s, 3H).

### Example 411: 2-(5-Fluoropyridin-2-yl)-6,6-bis(methyl-d₃)-3-(1H-pyrazolo[3,4-b]pyridin-4-yl)-6,7-dihydro-4H-pyrazolo[5,1-c][1,4]oxazine-4,4,7,7-d₄

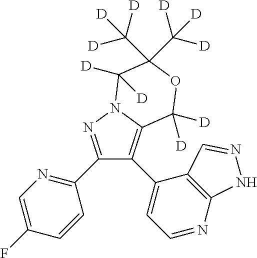

The title compound was prepared in a manner analogous to Example 1, Steps A-B, except using 3-bromo-2-(5-fluoropyridin-2-yl)-6,6-bis(methyl-d₃)-6,7-dihydro-4H-pyrazolo[5,1-c][1,4]oxazine-4,4,7,7-d₄ (Intermediate 184) instead of 3-bromo-2-(5-fluoropyridin-2-yl)-6,7-dihydro-4H-pyrazolo[5,1-c][1,4]oxazine (Intermediate 37) and 4-(4,4,5,5-tetramethyl-1,3,2-dioxaborolan-2-yl)-1-((2-(trimethylsilyl)ethoxy)methyl)-1H-pyrazolo[3,4-b]pyridine (Intermediate 23) instead of 4-(4,4,5,5-tetramethyl-1,3,2-dioxaborolan-2-yl)-1-((2-(trimethylsilyl)ethoxy)methyl)-1H-pyrrolo[2,3-b]pyridine (Intermediate 21) and heating at 90° C. via microwave irradiation for 1 hour for Step A. LCMS (ESI): mass calcd. for C₁₉H₇D₁₀FN₆ 374.2; m/z found 375.2 [M+1]⁺. ¹H NMR (400 MHz, DMSO-d₆) δ 13.51 (s, 1H), 8.45 (d, J=4.9 Hz, 1H), 8.22 (d, J=2.6 Hz, 1H), 8.02-7.83 (m, 1H), 7.82-7.62 (m, 1H), 7.27 (s, 1H), 7.09 (d, J=4.9 Hz, 1H).

### Example 412: (*R)-2-(4-Fluorophenyl)-6-methyl-3-(pyrazolo[1,5-a]pyridin-5-yl)-6-(trifluoromethyl)-6,7-dihydro-4H-pyrazolo[5,1-c][1,4]oxazine

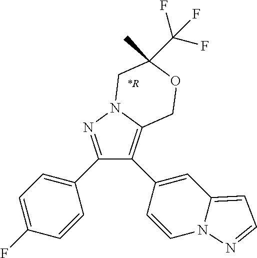 [SMILES: C[C@]1(C(F)(F)F)Cn2nc(-c3ccc(F)cc3)c(-c3ccn4nccc4c3)c2CO1]

3-Bromo-2-(4-fluorophenyl)-6-methyl-6-(trifluoromethyl)-6,7-dihydro-4H-pyrazolo[5,1-c][1,4]oxazine (Intermediate 185, 100 mg, 0.264 mmol), 5-(4,4,5,5-tetramethyl-1,3,2-dioxaborolan-2-yl)pyrazolo[1,5-a]pyridine (Intermediate 224) (71 mg, 0.29 mmol), Cs₂CO₃ (215 mg, 0.660 mmol), 2-methyl-2-butanol (4 mL), and H₂O (1 mL) were added to a 20 mL microwave tube. The resultant mixture was spared with Ar for 5 minutes and then treated with CataCXium® A-Pd-G3 (19 mg, 0.026 mmol). The resultant mixture stirred and heated at 100° C. via microwave irradiation for 1 hour. The reaction mixture was cooled to room-temperature. The solvent was removed under reduced pressure. The resulting residue was purified by FCC (SiO₂, eluent: petroleum ether:ethyl acetate=10:1 to 2:1) to afford the title compound (115 mg, 99%) as a yellow oil. MS (ESI): mass calcd. For C₂₁H₁₆F₄N_{4O} 416.1; m/z found 417.0 [M+H]⁺. Purification by SFC over DAICEL CHIRALPAK IG 250 mm×30 mm, 10 μm (isocratic elution: EtOH (containing 0.1% of 25% aq. NH₃): supercritical CO₂, 30%: 70% to 30%: 70% (v/v)) afforded the title compound (23.7 mg, 20%) as a white solid and (*S)-2-(4-fluorophenyl)-6-methyl-3-(pyrazolo[1,5-a]pyridin-5-yl)-6-(trifluoromethyl)-6,7-dihydro-4H-pyrazolo[5,1-c][1,4]oxazine (Example 413). MS (ESI): mass calcd. For C₂₁H₁₆F₄N_{4O} 416.1; m/z found 417.0 [M+H]⁺. ¹H NMR (400 MHz, DMSO-d₆) δ 8.61 (d, J=7.0 Hz, 1H), 7.99 (d, J=2.3 Hz, 1H), 7.58 (s, 1H), 7.52-7.42 (m, 2H), 7.25-7.15 (m, 2H), 6.61-6.49 (m, 2H), 5.23-5.06 (m, 2H), 4.44 (s, 2H), 1.58 (s, 3H).

### Example 413: (*S)-2-(4-Fluorophenyl)-6-methyl-3-(pyrazolo[1,5-a]pyridin-5-yl)-6-(trifluoromethyl)-6,7-dihydro-4H-pyrazolo[5,1-c][1,4]oxazine

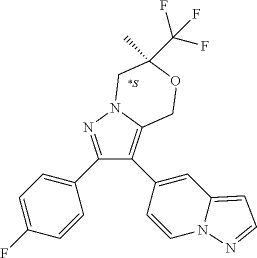 [SMILES: C[C@@]1(C(F)(F)F)Cn2nc(-c3ccc(F)cc3)c(-c3ccn4nccc4c3)c2CO1]

The title compound was isolated from chiral SFC purification of Example 412. MS (ESI): mass calcd. For C₂₁H₁₆F₄N_{4O} 416.1; m/z found 417.0 [M+H]⁺. ¹H NMR (400 MHz, DMSO-d₆) δ 8.61 (d, J=7.3 Hz, 1H), 7.99 (d, J=2.3 Hz, 1H), 7.60-7.55 (m, 1H), 7.51-7.43 (m, 2H), 7.25-7.17 (m, 2H), 6.60-6.48 (m, 2H), 5.22-5.06 (m, 2H), 4.44 (s, 2H), 1.58 (s, 3H).

### Example 414: (*R)-2-(5-Fluoropyridin-2-yl)-6-methyl-3-(pyrazolo[1,5-a]pyridin-5-yl)-6-(trifluoromethyl)-6,7-dihydro-4H-pyrazolo[5,1-c][1,4]oxazine

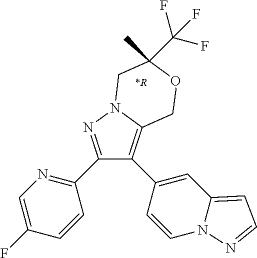 [SMILES: C[C@]1(C(F)(F)F)Cn2nc(-c3ccc(F)cn3)c(-c3ccn4nccc4c3)c2CO1]

(*R)-3-bromo-2-(5-fluoropyridin-2-yl)-6-methyl-6-(trifluoromethyl)-6,7-dihydro-4H-pyrazolo[5,1-c][1,4]oxazine (Intermediate 187, 40 mg, 0.11 mmol), 5-(4,4,5,5-tetramethyl-1,3,2-dioxaborolan-2-yl)pyrazolo[1,5-a]pyridine (Intermediate 224) (31 mg, 0.13 mmol), and Cs₂CO₃ (103 mg, 0.316 mmol) were added to a 10 mL microwave tube and the resulting mixture dissolved in 2-methyl-2-butanol (1.2 mL) and H₂O (0.3 mL). The resultant mixture was sparged with N₂ for 5 minutes and then treated with CataCXium® A-Pd-G3 (15 mg, 0.02 mmol). The resultant mixture was sparged with N₂ for another 5 minutes and heated at 100° C. via microwave irradiation for 1 hour. The reaction mixture was cooled to room-temperature. The reaction was quenched with H₂O (20 mL) and extracted with ethyl acetate (20 mL×3). The combined organic extracts were dried over anhydrous Na₂SO₄, filtered and concentrated to dryness under reduced pressure to afford the crude title product, which was further purified by preparative HPLC using a Waters Xbridge BEH C18 100×25 mm×5 μm column (eluent: 35% to 75% (v/v) CH₃CN and H₂O with 0.05% NH₃) to yield the title compound, after lyophilization, as a white solid (23.9 mg, 53%). MS (ESI): mass calcd. for C₂₀H₁₅F₄N₅O 417.1; m/z found 418.1 [M+H]⁺. ¹H NMR (400 MHz, DMSO-d₆) δ 8.54 (d, J=7.0 Hz, 1H), 8.45 (d, J=3.0 Hz, 1H), 7.97 (d, J=2.3 Hz, 1H), 7.89-7.84 (m, 1H), 7.83-7.75 (m, 1H), 7.61-7.57 (m, 1H), 6.62-6.57 (m, 1H), 6.56-6.53 (m, 1H), 5.23-5.07 (m, 2H), 4.47 (s, 2H), 1.58 (s, 3H).

### Example 415: (*S)-2-(5-Fluoropyridin-2-yl)-6-methyl-3-(pyrazolo[1,5-a]pyridin-5-yl)-6-(trifluoromethyl)-6,7-dihydro-4H-pyrazolo[5,1-c][1,4]oxazine

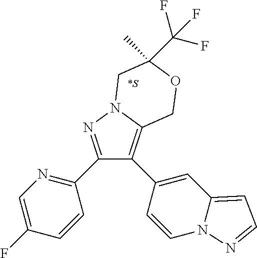 [SMILES: C[C@@]1(C(F)(F)F)Cn2nc(-c3ccc(F)cn3)c(-c3ccn4nccc4c3)c2CO1]

The title compound was prepared in a manner analogous to Example 414 except using (*S)-3-bromo-2-(5-fluoropyridin-2-yl)-6-methyl-6-(trifluoromethyl)-6,7-dihydro-4H-pyrazolo[5,1-c][1,4]oxazine (Intermediate 188) instead of (*R)-3-bromo-2-(5-fluoropyridin-2-yl)-6-methyl-6-(trifluoromethyl)-6,7-dihydro-4H-pyrazolo[5,1-c][1,4]oxazine (Intermediate 187). MS (ESI): mass calcd. for C₂₀H₁₅F₄N₅O 417.1; m/z found 418.1 [M+H]⁺. ¹H NMR (400 MHz, DMSO-d₆) δ 8.54 (d, J=7.3 Hz, 1H), 8.45 (d, J=2.8 Hz, 1H), 7.97 (d, J=2.3 Hz, 1H), 7.89-7.84 (m, 1H), 7.83-7.76 (m, 1H), 7.61-7.57 (m, 1H), 6.62-6.57 (m, 1H), 6.56-6.52 (m, 1H), 5.22-5.07 (m, 2H), 4.47 (s, 2H), 1.58 (s, 3H).

### Example 416: (*R)-2-(4-Fluorophenyl)-6-methyl-3-(1H-pyrazolo[3,4-b]pyridin-4-yl)-6-(trifluoromethyl)-6,7-dihydro-4H-pyrazolo[5,1-c][1,4]oxazine

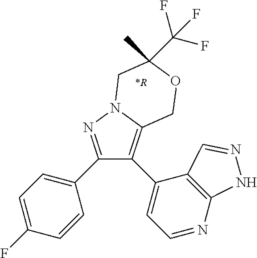 [SMILES: C[C@]1(C(F)(F)F)Cn2nc(-c3ccc(F)cc3)c(-c3ccnc4[nH]ncc34)c2CO1]

The title compound was prepared in a manner analogous to Example 1, Steps A-B, except using 3-bromo-2-(4-fluorophenyl)-6-methyl-6-(trifluoromethyl)-6,7-dihydro-4H-pyrazolo[5,1-c][1,4]oxazine (Intermediate 185) instead of 3-bromo-2-(5-fluoropyridin-2-yl)-6,7-dihydro-4H-pyrazolo[5,1-c][1,4]oxazine (Intermediate 37) and 4-(4,4,5,5-tetramethyl-1,3,2-dioxaborolan-2-yl)-1-((2-(trimethylsilyl)ethoxy)methyl)-1H-pyrazolo[3,4-b]pyridine (Intermediate 23) instead of 4-(4,4,5,5-tetramethyl-1,3,2-dioxaborolan-2-yl)-1-((2-(trimethylsilyl)ethoxy)methyl)-1H-pyrrolo[2,3-b]pyridine (Intermediate 21) and Pd(dppf)Cl₂ instead of CataCXium® A Pd G3 and microwave irradiation at 100° C. for one hour instead of conventional heating in Step A. chiral SFC purification (Phenomenex-Cellulose-2 250 mm×30 mm, 10 μm (isocratic elution: EtOH (containing 0.1% of 25% aq. NH₃): supercritical CO₂, 45%: 55% to 45%: 55% (v/v))) afforded the title compound. MS (ESI): mass calcd. for C₂₀H₁₅F₄N₅O 417.1; m/z found 418.0 [M+H]⁺. ¹H NMR (400 MHz, DMSO-d₆) δ 8.50 (d, J=4.5 Hz, 1H), 7.39-7.30 (m, 3H), 7.16-7.06 (m, 3H), 5.16-4.97 (m, 2H), 4.50 (s, 2H), 1.60 (s, 3H); and (*S)-2-(4-fluorophenyl)-6-methyl-3-(1H-pyrazolo[3,4-b]pyridin-4-yl)-6-(trifluoromethyl)-6,7-dihydro-4H-pyrazolo[5,1-c][1,4]oxazine (Example 417).

### Example 417: (*)-2-(4-Fluorophenyl)-6-methyl-3-(1H-pyrazolo[3,4-b]pyridin-4-yl)-6-(trifluoromethyl)-6,7-dihydro-4H-pyrazolo[5,1-c][1,4]oxazine

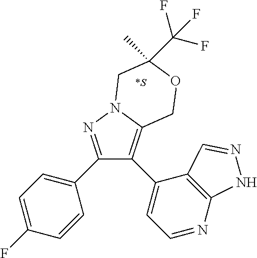 [SMILES: C[C@@]1(C(F)(F)F)Cn2nc(-c3ccc(F)cc3)c(-c3ccnc4[nH]ncc34)c2CO1]

The title compound was isolated from chiral SFC purification of Example 416. MS (ESI): mass calcd. for C₂₀H₁₅F₄N₅O 417.1; m/z found 418.0 [M+H]⁺. ¹H NMR (400 MHz, DMSO-d₆) δ 13.63 (s, 1H), 8.51 (d, J=4.8 Hz, 1H), 7.39-7.30 (m, 3H), 7.16-7.07 (m, 3H), 5.16-4.98 (m, 2H), 4.50 (s, 2H), 1.60 (s, 3H).

### Example 418: (*R)-2-(5-Fluoropyridin-2-yl)-6-methyl-3-(3-methyl-1H-pyrazolo[3,4-b]pyridin-4-yl)-6-(trifluoromethyl)-6,7-dihydro-4H-pyrazolo[5,1-c][1,4]oxazine

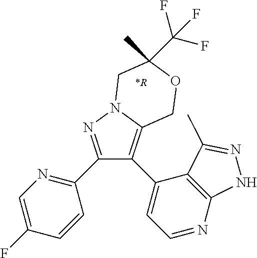 [SMILES: Cc1n[nH]c2nccc(-c3c(-c4ccc(F)cn4)nn4c3CO[C@@](C)(C(F)(F)F)C4)c12]

The title compound was prepared in a manner analogous to Example 430, Steps A-C, except using 3-bromo-2-(5-fluoropyridin-2-yl)-6-methyl-6-(trifluoromethyl)-6,7-dihydro-4H-pyrazolo[5,1-c][1,4]oxazine (Intermediate 186) instead of 3-bromo-2-(5-fluoropyridin-2-yl)-6-(methyl-d₃)-6-(trifluoromethyl)-6,7-dihydro-4H-pyrazolo[5,1-c][1,4]oxazine (Intermediate 189 and 4-chloro-3-methyl-1-((2-(trimethylsilyl)ethoxy)methyl)-1H-pyrazolo[3,4-b]pyridine (Intermediate 203) instead of 4-bromo-5-fluoro-3,6-dimethyl-1-((2-(trimethylsilyl)ethoxy)methyl)-1H-pyrazolo[3,4-b]pyridine (Intermediate 209) and purification by HPLC using a Boston Prime C18, 150 mm×30 mm×5 μm column (eluent: 60% to 90% (v/v) CH₃CN and H₂O with 0.05% NH₃) instead of FCC for Step A. Purification via SFC over DAICEL CHIRALPAK AD (250 mm×30 mm×10 m) (isocratic elution: EtOH (containing 0.1% aq. NH₃): supercritical CO₂, 25%: 75% to 25%: 75% (v/v)) was used for Step C. MS (ESI): mass calcd. for C₂₀H₁₆F₄N₆O 432.1; m/z found 433.1 [M+H]⁺. ¹H NMR (400 MHz, DMSO-d₆) δ 13.19-13.12 (m, 1H), 8.41 (d, J=4.6 Hz, 1H), 8.13-8.09 (m, 1H), 7.90-7.83 (m, 1H), 7.73-7.65 (m, 1H), 6.98 (dd, J=4.6, 10.4 Hz, 1H), 5.00 (t, J=16.3 Hz, 1H), 4.79-4.65 (m, 1H), 4.60-4.46 (m, 2H), 1.85-1.76 (m, 3H), 1.61-1.46 (m, 3H).

### Example 419: (*S)-2-(5-Fluoropyridin-2-yl)-6-methyl-3-(3-methyl-1H-pyrazolo[3,4-b]pyridin-4-yl)-6-(trifluoromethyl)-6,7-dihydro-4H-pyrazolo[5,1-c][1,4]oxazine

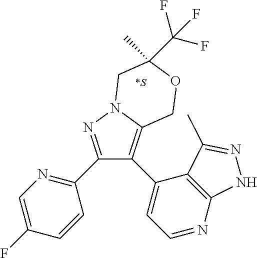 [SMILES: Cc1n[nH]c2nccc(-c3c(-c4ccc(F)cn4)nn4c3CO[C@](C)(C(F)(F)F)C4)c12]

The title compound was isolated from chiral SFC purification of Example 418. MS (ESI): mass calcd. for C₂₀H₁₆F₄N₆O 432.1; m/z found 433.1 [M+H]⁺. ¹H NMR (400 MHz, DMSO-d₆) δ13.24
13.06 (m, 1H), 8.40 (d, J=4.6 Hz, 1H), 8.15-8.08 (m, 1H), 7.91-7.81 (m, 1H), 7.74-7.64 (m, 1H), 7.02-6.94 (m, 1H), 5.00 (t, J=16.6 Hz, 1H), 4.79-4.64 (m, 1H), 4.60-4.46 (m, 2H), 1.86-1.76 (m, 3H), 1.60-1.46 (m, 3H).

### Example 420: (*R)-3-(5-Fluoro-6-methyl-1H-pyrazolo[3,4-b]pyridin-4-yl)-2-(5-fluoropyridin-2-yl)-6-methyl-6-(trifluoromethyl)-6,7-dihydro-4H-pyrazolo[5,1-c][1,4]oxazine

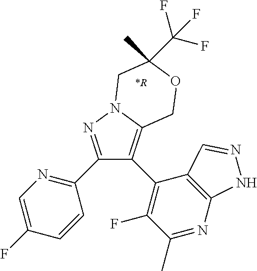 [SMILES: Cc1nc2[nH]ncc2c(-c2c(-c3ccc(F)cn3)nn3c2CO[C@@](C)(C(F)(F)F)C3)c1F]

The title compound was prepared in a manner analogous to Example 1, Steps A-B, except using (*R)-3-bromo-2-(5-fluoropyridin-2-yl)-6-methyl-6-(trifluoromethyl)-6,7-dihydro-4H-pyrazolo[5,1-c][1,4]oxazine (Intermediate 187) instead of 3-bromo-2-(5-fluoropyridin-2-yl)-6,7-dihydro-4H-pyrazolo[5,1-c][1,4]oxazine (Intermediate 37) and 5-fluoro-6-methyl-4-(4,4,5,5-tetramethyl-1,3,2-dioxaborolan-2-yl)-1-((2-(trimethylsilyl)ethoxy)methyl)-1H-pyrazolo[3,4-b]pyridine (Intermediate 223) instead of 4-(4,4,5,5-tetramethyl-1,3,2-dioxaborolan-2-yl)-1-((2-(trimethylsilyl)ethoxy)methyl)-1H-pyrrolo[2,3-b]pyridine (Intermediate 21) in Step A. Purification via preparative HPLC using a Phenomenex Gemini-NX C18 75 mm×30 mm×3 m (eluent: 40% to 70% (v/v) CH₃CN and H₂O with 0.05% NH₃) and lyophilization yielded the title compound as a white solid. MS (ESI): mass calcd. for C₂₀H₁₅F₅N₆O 450.1; m/z found 451.0 [M+H]⁺. ¹H NMR (400 MHz, CH₃₀D) δ 8.15 (s, 1H), 7.95-7.87 (m, 1H), 7.63 (dt, J=3.0, 8.6 Hz, 1H), 7.46-7.33 (m, 1H), 5.08-4.96 (m, 2H), 4.66-4.57 (m, 1H), 4.51-4.41 (m, 1H), 2.64 (d, J=3.6 Hz, 3H), 1.70-1.60 (m, 3H). N—H proton is not observed.

### Example 421: (*S)-3-(5-Fluoro-6-methyl-1H-pyrazolo[3,4-b]pyridin-4-yl)-2-(5-fluoropyridin-2-yl)-6-methyl-6-(trifluoromethyl)-6,7-dihydro-4H-pyrazolo[5,1-c][1,4]oxazine

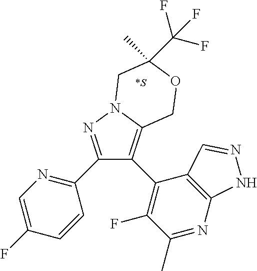 [SMILES: Cc1nc2[nH]ncc2c(-c2c(-c3ccc(F)cn3)nn3c2CO[C@](C)(C(F)(F)F)C3)c1F]

The title compound was prepared in a manner analogous to Example 1, Steps A-B, except using (*S)-3-bromo-2-(5-fluoropyridin-2-yl)-6-methyl-6-(trifluoromethyl)-6,7-dihydro-4H-pyrazolo[5,1-c][1,4]oxazine (Intermediate 188) instead of 3-bromo-2-(5-fluoropyridin-2-yl)-6,7-dihydro-4H-pyrazolo[5,1-c][1,4]oxazine (Intermediate 37) and 5-fluoro-6-methyl-4-(4,4,5,5-tetramethyl-1,3,2-dioxaborolan-2-yl)-1-((2-(trimethylsilyl)ethoxy)methyl)-1H-pyrazolo[3,4-b]pyridine (Intermediate 223) instead of 4-(4,4,5,5-tetramethyl-1,3,2-dioxaborolan-2-yl)-1-((2-(trimethylsilyl)ethoxy)methyl)-1H-pyrrolo[2,3-b]pyridine (Intermediate 21) in Step A. Purification via preparative HPLC using a Phenomenex Gemini-NX C18 75 mm×30 mm×3 m (eluent: 40% to 70% (v/v) CH₃CN and H₂O with 0.05% NH₃) and lyophilization yielded the title compound as a white solid. MS (ESI): mass calcd. for C₂₀H₁₅F₅N₆O 450.1; m/z found 451.0 [M+H]⁺. ¹H NMR (400 MHz, CH₃₀D) δ 8.15 (d, J=2.7 Hz, 1H), 7.94-7.87 (m, 1H), 7.63 (dt, J=2.9, 8.6 Hz, 1H), 7.44-7.34 (m, 1H), 5.10-4.95 (m, 2H), 4.63-4.57 (m, 1H), 4.51-4.40 (m, 1H), 2.64 (d, J=3.6 Hz, 3H), 1.70-1.60 (m, 3H). N—H proton not observed.

### Example 422: 2-(4-Fluorophenyl)-6-methyl-3-(6-methyl-1H-pyrazolo[3,4-d]pyrimidin-4-yl)-6-(trifluoromethyl)-6,7-dihydro-4H-pyrazolo[5,1-c][1,4]oxazine

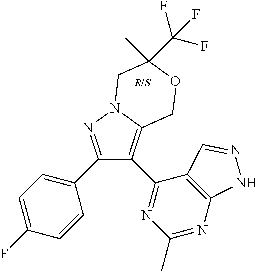 [SMILES: Cc1nc(-c2c(-c3ccc(F)cc3)nn3c2COC(C)(C(F)(F)F)C3)c2cn[nH]c2n1]

The title compound was prepared in a manner analogous to Example 430, Steps A-B, except using 3-bromo-2-(4-fluorophenyl)-6-methyl-6-(trifluoromethyl)-6,7-dihydro-4H-pyrazolo[5,1-c][1,4]oxazine (Intermediate 185) instead of 3-bromo-2-(5-fluoropyridin-2-yl)-6-(methyl-d₃)-6-(trifluoromethyl)-6,7-dihydro-4H-pyrazolo[5,1-c][1,4]oxazine (Intermediate 189) and 4-chloro-6-methyl-1-((2-(trimethylsilyl)ethoxy)methyl)-1H-pyrazolo[3,4-d]pyrimidine (Intermediate 185) instead of 4-bromo-5-fluoro-3,6-dimethyl-1-((2-(trimethylsilyl)ethoxy)methyl)-1H-pyrazolo[3,4-b]pyridine (Intermediate 209) and microwave irradiation at 90° C. for 1 hour instead of conventional heating for Step A. MS (ESI): mass calcd.

For C₂₀H₁₆F₄N₆O 432.1; m/z found 433.3 [M+H]⁺. ¹H NMR (400 MHz, DMSO-d₆) δ 13.67 (br s, 1H), 7.52-7.44 (m, 2H), 7.27-7.17 (m, 2H), 6.69 (s, 1H), 5.36-5.20 (m, 2H), 4.55-4.45 (m, 2H), 2.70 (s, 3H), 1.58 (s, 3H).

### Example 423: (*R)-2-(5-Fluoropyridin-2-yl)-6-(methyl-d₃)-3-(1H-pyrazolo[3,4-b]pyridin-4-yl)-6-(trifluoromethyl)-6,7-dihydro-4H-pyrazolo[5,1-c][1,4]oxazine

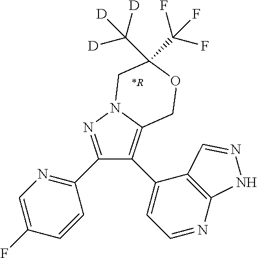 [SMILES: [2H]C([2H])([2H])[C@]1(C(F)(F)F)Cn2nc(-c3ccc(F)cn3)c(-c3ccnc4[nH]ncc34)c2CO1]

The title compound was prepared in a manner analogous to Example 1, Steps A-B, except using 3-bromo-2-(5-fluoropyridin-2-yl)-6-(methyl-d₃)-6-(trifluoromethyl)-6,7-dihydro-4H-pyrazolo[5,1-c][1,4]oxazine (Intermediate 189) instead of 3-bromo-2-(5-fluoropyridin-2-yl)-6,7-dihydro-4H-pyrazolo[5,1-c][1,4]oxazine (Intermediate 37) and 4-(4,4,5,5-tetramethyl-1,3,2-dioxaborolan-2-yl)-1-((2-(trimethylsilyl)ethoxy)methyl)-1H-pyrazolo[3,4-b]pyridine (Intermediate 23) instead of 4-(4,4,5,5-tetramethyl-1,3,2-dioxaborolan-2-yl)-1-((2-(trimethylsilyl)ethoxy)methyl)-1H-pyrrolo[2,3-b]pyridine (Intermediate 21) in Step A. The title compound was separated from its enantiomer using chiral SFC over DAICEL CHIRALPAK AD (250 mm×30 mm×10 um (isocratic elution: EtOH (containing 0.1% of 25% aq. NH₃): supercritical CO₂, 45%: 55% to 45%: 55% (v/v)) and the desired fractions were concentrated under reduce pressure. Additional purification by preparative HPLC using a Boston Prime C18 150×30 mm×5 μm column (eluent: 30% to 60% (v/v) CH₃CN and H₂O with 0.05% NH₃H₂O+10 mM NH₄HCO₃) afforded the title compound as a white solid: MS (ESI): mass calcd. for C₁₉H₁₁D₃F₄N₆O 421.1; m/z found 422.1 [M+H]⁺. ¹H NMR (400 MHz, DMSO-d₆) δ 13.55 (br s, 1H), 8.47 (d, J=4.8 Hz, 1H), 8.24 (d, J=3.0 Hz, 1H), 7.90-7.86 (m, 1H), 7.82-7.76 (m, 1H), 7.29 (s, 1H), 7.09 (d, J=4.8 Hz, 1H), 5.15-5.09 (m, 1H), 5.05-4.98 (m, 1H), 4.53 (s, 2H) and (*S)-2-(5-fluoropyridin-2-yl)-6-(methyl-d₃)-3-(1H-pyrazolo[3,4-b]pyridin-4-yl)-6-(trifluoromethyl)-6,7-dihydro-4H-pyrazolo[5,1-c][1,4]oxazine (Example 424).

### Example 424: (*S)-2-(5-Fluoropyridin-2-yl)-6-(methyl-d₃)-3-(1H-pyrazolo[3,4-b]pyridin-4-yl)-6-(trifluoromethyl)-6,7-dihydro-4H-pyrazolo[5,1-c][1,4]oxazine

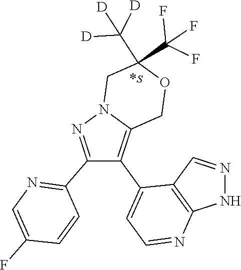

The title compound was isolated from chiral SFC purification of Example 423. MS (ESI): mass calcd. for C₁₉H₁₁D₃F₄N₆O 421.1; m/z found 422.1 [M+H]⁺. ¹H NMR (400 MHz, DMSO-d₆) δ 11.93 (s, 1H), 8.60 (d, J=4.8 Hz, 1H), 8.28 (d, J=2.7 Hz, 1H), 7.55 (dd, J=4.4, 8.8 Hz, 1H), 7.48 (s, 1H), 7.35-7.29 (m, 1H), 7.01 (d, J=4.8 Hz, 1H), 5.08-5.02 (m, 1H), 4.96-4.91 (m, 1H), 4.57-4.53 (m, 1H), 4.32-4.27 (m, 1H).

### Example 425: (*R)-2-(5-Fluoropyridin-2-yl)-6-(methyl-d₃)-3-(3-methyl-1H-pyrazolo[3,4-b]pyridin-4-yl)-6-(trifluoromethyl)-6,7-dihydro-4H-pyrazolo[5,1-c][1,4]oxazine

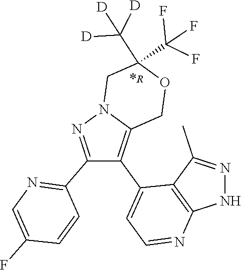

The title compound was prepared in a manner analogous to Example 1, Steps A-B, except using 3-bromo-2-(5-fluoropyridin-2-yl)-6-(methyl-d₃)-6-(trifluoromethyl)-6,7-dihydro-4H-pyrazolo[5,1-c][1,4]oxazine (Intermediate 189) instead of 3-bromo-2-(5-fluoropyridin-2-yl)-6,7-dihydro-4H-pyrazolo[5,1-c][1,4]oxazine (Intermediate 37) and 3-methyl-4-(4,4,5,5-tetramethyl-1,3,2-dioxaborolan-2-yl)-1-((2-(trimethylsilyl)ethoxy)methyl)-1H-pyrazolo[3,4-b]pyridine (Intermediate 220) instead of 4-(4,4,5,5-tetramethyl-1,3,2-dioxaborolan-2-yl)-1-((2-(trimethylsilyl)ethoxy)methyl)-1H-pyrrolo[2,3-b]pyridine (Intermediate 21) in Step A. Purification (chiral SFC over DAICEL CHIRALPAK IG (250 mm×30 mm×10 um (isocratic elution: EtOH (containing 0.1% of 25% aq. NH₃): supercritical CO₂, 60%: 40% to 60%: 40% (v/v)) afforded the title compound: MS (ESI): mass calcd. for C₂₀H₁₃D₃F₄N₆O 435.2 m/z found 436.1 [M+H]⁺; ¹H NMR (400 MHz, DMSO-d₆) δ 13.19-13.15 (m, 1H), 8.43 (d, J=4.5 Hz, 1H), 8.14 (d, J=2.8 Hz, 1H), 7.92-7.85 (m, 1H), 7.74-7.68 (m, 1H), 7.00 (dd, J=4.5, 10.5 Hz, 1H), 5.02 (t, J=16.6 Hz, 1H), 4.80-4.68 (m, 1H), 4.61-4.49 (m, 2H), 1.84 (d, J=19.8 Hz, 3H) and (*S)-2-(5-fluoropyridin-2-yl)-6-(methyl-d₃)-3-(3-methyl-1H-pyrazolo[3,4-b]pyridin-4-yl)-6-(trifluoromethyl)-6,7-dihydro-4H-pyrazolo[5,1-c][1,4]oxazine (Example 426).

### Example 426: (*S)-2-(5-Fluoropyridin-2-yl)-6-(methyl-d₃)-3-(3-methyl-1H-pyrazolo[3,4-b]pyridin-4-yl)-6-(trifluoromethyl)-6,7-dihydro-4H-pyrazolo[5,1-c][1,4]oxazine

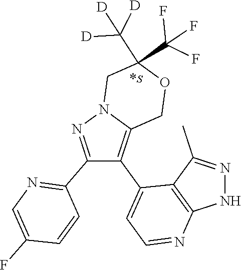

The title compound was isolated from chiral SFC purification of Example 425. MS (ESI): mass calcd. for C₂₀H₁₃D₃F₄N₆O 435.2 m/z found 436.1 [M+H]⁺. ¹H NMR (400 MHz, DMSO-d₆) δ 13.19-13.14 (m, 1H), 8.43 (d, J=4.5 Hz, 1H), 8.14 (d, J=2.5 Hz, 1H), 7.92-7.86 (m, 1H), 7.75-7.68 (m, 1H), 7.00 (dd, J=4.6, 10.4 Hz, 1H), 5.02 (t, J=16.6 Hz, 1H), 4.80-4.68 (m, 1H), 4.62-4.49 (m, 2H), 1.84 (d, J=20.0 Hz, 3H).

### Example 427: (*S)-3-(3-Chloro-1H-pyrazolo[3,4-b]pyridin-4-yl)-2-(5-fluoropyridin-2-yl)-6-(methyl-d₃)-6-(trifluoromethyl)-6,7-dihydro-4H-pyrazolo[5,1-c][1,4]oxazine

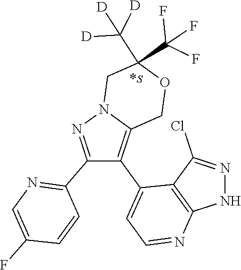

The title compound was prepared in a manner analogous to Example 224, except using (*S)-2-(5-fluoropyridin-2-yl)-6-(methyl-d₃)-3-(1H-pyrazolo[3,4-b]pyridin-4-yl)-6-(trifluoromethyl)-6,7-dihydro-4H-pyrazolo[5,1-c][1,4]oxazine (Example 424) instead of 5-fluoro-4-(2-(5-fluoropyridin-2-yl)-5,6-dihydro-4H-pyrrolo[1,2-b]pyrazol-3-yl)-6-methyl-1H-pyrazolo[3,4-b]pyridine (Example 9). MS (ESI): mass calcd. for C₁₉H₁₀D₃ClF₄N₆O, 455.1; m/z found, 456.1 [M+H]⁺. ¹H NMR (400 MHz, CD₃OD): δ 8.53 (dd, J=4.7, 1.9 Hz, 1H), 8.04 (dd, J=5.1, 2.9 Hz, 1H), 7.93-7.77 (m, 1H), 7.62-7.45 (m, 1H), 7.12 (dd, J=4.7, 2.7 Hz, 1H), 5.04 (t, J=16.3 Hz, 1H), 4.86 (d, J=1.9 Hz, 1H), 4.55 (dd, J=13.3, 7.1 Hz, 1H), 4.41 (dd, J=13.3, 9.5 Hz, 1H). N—H proton not observed.

### Example 428: (*R)-3-(3,6-Dimethyl-1H-pyrazolo[3,4-b]pyridin-4-yl)-2-(5-fluoropyridin-2-yl)-6-(methyl-d₃)-6-(trifluoromethyl)-6,7-dihydro-4H-pyrazolo[5,1-c][1,4]oxazine

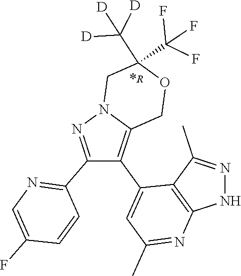

The title compound was prepared in a manner analogous to Example 1, Steps A-B, except using 3-bromo-2-(5-fluoropyridin-2-yl)-6-(methyl-d₃)-6-(trifluoromethyl)-6,7-dihydro-4H-pyrazolo[5,1-c][1,4]oxazine (Intermediate 189) instead of 3-bromo-2-(5-fluoropyridin-2-yl)-6,7-dihydro-4H-pyrazolo[5,1-c][1,4]oxazine (Intermediate 37) and 1-(4-methoxybenzyl)-3,6-dimethyl-4-(4,4,5,5-tetramethyl-1,3,2-dioxaborolan-2-yl)-1H-pyrazolo[3,4-b]pyridine (Intermediate 218) instead of 4-(4,4,5,5-tetramethyl-1,3,2-dioxaborolan-2-yl)-1-((2-(trimethylsilyl)ethoxy)methyl)-1H-pyrrolo[2,3-b]pyridine (Intermediate 21) in Step A. Purification (chiral SFC over DAICEL CHIRALPAK AD (250 mm×30 mm×10 um (isocratic elution: IPA (containing 0.1% of 25% aq. NH₃): supercritical CO₂, 40%: 60% to 40%: 60% (v/v)) afforded the title compound: MS (ESI): mass calcd. for C₂₁H₁₅D₃F₄N₆O 449.2 m/z, found 450.1 [M+H]⁺; ¹H NMR (400 MHz, DMSO-d₆): δ 12.94 (d, J=2.3 Hz, 1H), 8.18-8.14 (m, 1H), 7.90-7.84 (m, 1H), 7.74-7.67 (m, 1H), 6.90 (d, J=12.4 Hz, 1H), 5.06 (t, J=15.6 Hz, 1H), 4.79-4.67 (m, 1H), 4.60-4.48 (m, 2H), 2.55 (s, 3H), 1.78 (d, J=19.3 Hz, 3H) and (*S)-3-(3,6-Dimethyl-1H-pyrazolo[3,4-b]pyridin-4-yl)-2-(5-fluoropyridin-2-yl)-6-(methyl-d₃)-6-(trifluoromethyl)-6,7-dihydro-4H-pyrazolo[5,1-c][1,4]oxazine (Example 429).

### Example 429: (*S)-3-(3,6-Dimethyl-1H-pyrazolo[3,4-b]pyridin-4-yl)-2-(5-fluoropyridin-2-yl)-6-(methyl-d₃)-6-(trifluoromethyl)-6,7-dihydro-4H-pyrazolo[5,1-c][1,4]oxazine

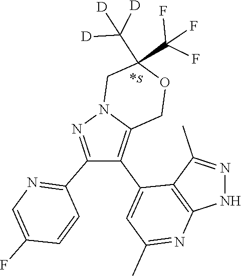

The title compound was isolated from chiral SFC purification of Example 428. MS (ESI): mass calcd. for C₂₁H₁₅D₃F₄N₆O 449.2 m/z, found 450.1 [M+H]⁺. ¹H NMR (400 MHz, DMSO-d₆) δ 12.94 (d, J=2.1 Hz, 1H), 8.18-8.14 (m, 1H), 7.90-7.84 (m, 1H), 7.74-7.67 (m, 1H), 6.90 (d, J=12.4 Hz, 1H), 5.05 (t, J=15.6 Hz, 1H), 4.78-4.67 (m, 1H), 4.60-4.49 (m, 2H), 2.55 (s, 3H), 1.78 (d, J=19.3 Hz, 3H).

### Example 430: (*R)-3-(5-Fluoro-3,6-dimethyl-1H-pyrazolo[3,4-b]pyridin-4-yl)-2-(5-fluoropyridin-2-yl)-6-(methyl-d₃)-6-(trifluoromethyl)-6,7-dihydro-4H-pyrazolo[5,1-c][1,4]oxazine

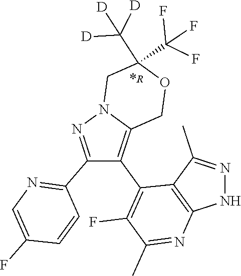

Step A: 3-(5-Fluoro-3,6-dimethyl-1-((2-(trimethylsilyl)ethoxy)methyl)-1H-pyrazolo[3,4-b]pyridin-4-yl)-2-(5-fluoropyridin-2-yl)-6-(methyl-d₃)-6-(trifluoromethyl)-6,7-dihydro-4H-pyrazolo[5,1-c][1,4]oxazine. 3-Bromo-2-(5-fluoropyridin-2-yl)-6-(methyl-d₃)-6-(trifluoromethyl)-6,7-dihydro-4H-pyrazolo[5,1-c][1,4]oxazine (Intermediate 189, 270 mg, 0.705 mmol), 4-bromo-5-fluoro-3,6-dimethyl-1-((2-(trimethylsilyl)ethoxy)methyl)-1H-pyrazolo[3,4-b]pyridine (Intermediate 209, 265 mg, 0.708 mmol), 4,4,4′,4′,5,5,5′,5′-octamethyl-2,2′-bi(1,3,2-dioxaborolane (270 mg, 1.06 mmol), and Cs₂CO₃ (690 mg, 2.12 mmol) were dissolved in 1,4-dioxane (8 mL) and H₂O (0.8 mL). The resultant mixture was sparged with N₂ for 5 minutes and then treated with CataCXium A-Pd-G2 (50 mg, 0.075 mmol). The mixture was sparged with N₂ for another 5 minutes and heated at 90° C. for 16 hours. The reaction mixture was concentrated to dryness under reduced pressure. The resulting residue was purified (FCC, SiO₂, eluent: petroleum ether:ethyl acetate=20:1 to 3:1) to afford the title compound (300 mg, 66%) as yellow solid. MS (ESI): mass calcd. for C₂₇H₂₈D₃F₅N₆O₂Si 597.2; m/z found 598.6 [M+H]⁺.

Step B: 3-(5-Fluoro-3,6-dimethyl-1H-pyrazolo[3,4-b]pyridin-4-yl)-2-(5-fluoropyridin-2-yl)-6-(methyl-d₃)-6-(trifluoromethyl)-6,7-dihydro-4H-pyrazolo[5,1-c][1,4]oxazine. TFA (3 mL) was added to a solution consisting of 3-(5-fluoro-3,6-dimethyl-1-((2-(trimethylsilyl)ethoxy)methyl)-1H-pyrazolo[3,4-b]pyridin-4-yl)-2-(5-fluoropyridin-2-yl)-6-(methyl-d₃)-6-(trifluoromethyl)-6,7-dihydro-4H-pyrazolo[5,1-c][1,4]oxazine (300 mg. 0.502 mmol) and dichloromethane (6 mL) at a 50 mL round-bottomed flask. The resultant mixture was stirred at room-temperature for 2 hours. The reaction mixture was concentrated to dryness under reduced pressure to afford the title compound, which was dissolved in MeOH (6 mL) and treated with 2 M NH₃ in methanol (3 mL). Then the mixture was stirred at room-temperature for 1 hour. The reaction mixture was concentrated to dryness under reduced pressure. The residue was diluted with H₂O (10 mL) and extracted with dichloromethane (15 mL×3). The combined organic extracts were dried over anhydrous Na₂SO₄, filtered, and concentrated to dryness under reduced pressure to give the impure product (200 mg), which was purified by preparative HPLC using a Phenomenex Gemini-NX C18 75×30 mm×3 m column (eluent: 37% to 67% (v/v) CH₃CN and H₂O with 0.05% NH₃+10 mM NH₄HCO₃) to afford the title compound, after lyophilization, as a white solid (50 mg, 21%). Chiral purification (SFC over DAICEL CHIRALPAK AD (250 mm×30 mm×10 um (isocratic elution: EtOH (containing 0.1% of 25% aq. NH₃): supercritical CO₂, 40%: 60% to 40%: 60% (v/v))) afforded the title compound: MS (ESI): mass calcd. for C₂₁H₁₄D₃F₅N₆O 467.2; m/z found 468.1 [M+H]⁺. ¹H NMR (400 MHz, DMSO-d₆): δ 13.11 (br s, 1H), 8.14 (d, J=2.9 Hz, 1H), 7.97 (dd, J=4.5, 8.9 Hz, 1H), 7.77-7.71 (m, 1H), 4.90-4.76 (m, 2H), 4.66-4.54 (m, 2H), 2.54 (d, J=3.3 Hz, 3H), 1.81 (s, 3H) and (*S)-3-(5-fluoro-3,6-dimethyl-1H-pyrazolo[3,4-b]pyridin-4-yl)-2-(5-fluoropyridin-2-yl)-6-(methyl-d₃)-6-(trifluoromethyl)-6,7-dihydro-4H-pyrazolo[5,1-c][1,4]oxazine (Example 431).

### Example 431: (*S)-3-(5-Fluoro-3,6-dimethyl-1H-pyrazolo[3,4-b]pyridin-4-yl)-2-(5-fluoropyridin-2-yl)-6-(methyl-d₃)-6-(trifluoromethyl)-6,7-dihydro-4H-pyrazolo[5,1-c][1,4]oxazine

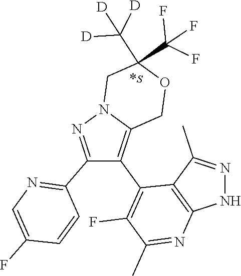

The title compound was isolated from chiral SFC purification of Example 430. MS (ESI): mass calcd. for C₂₁H₁₄D₃F₅N₆O 467.2; m/z found 468.1 [M+H]⁺. ¹H NMR (400 MHz, DMSO-d₆) δ 13.11 (s, 1H), 8.14 (d, J=2.9 Hz, 1H), 7.97 (dd, J=4.5, 8.9 Hz, 1H), 7.77-7.71 (m, 1H), 4.90-4.76 (m, 2H), 4.66-4.54 (m, 2H), 2.54 (d, J=3.5 Hz, 3H), 1.81 (s, 3H).

### Example 432: 2-(5-Fluoro-2-pyridyl)-3-(1H-pyrazolo[3,4-b]pyridin-4-yl)spiro[4,7-dihydropyrazolo[5,1-c][1,4]oxazine-6,1′-cyclopropane]

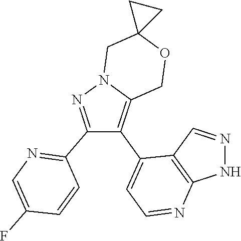 [SMILES: Fc1ccc(-c2nn3c(c2-c2ccnc4[nH]ncc24)COC2(CC2)C3)nc1]

The title compound was prepared in a manner analogous to Example 1, Steps A-B, except using 3′-bromo-2′-(5-fluoropyridin-2-yl)-4′,7′-dihydrospiro[cyclopropane-1,6′-pyrazolo[5,1-c][1,4]oxazine] (Intermediate 190) instead of 3-bromo-2-(5-fluoropyridin-2-yl)-6,7-dihydro-4H-pyrazolo[5,1-c][1,4]oxazine (Intermediate 37) and 4-(4,4,5,5-tetramethyl-1,3,2-dioxaborolan-2-yl)-1-((2-(trimethylsilyl)ethoxy)methyl)-1H-pyrazolo[3,4-b]pyridine (Intermediate 23) instead of 4-(4,4,5,5-tetramethyl-1,3,2-dioxaborolan-2-yl)-1-((2-(trimethylsilyl)ethoxy)methyl)-1H-pyrrolo[2,3-b]pyridine (Intermediate 21) in Step A. MS (ESI): mass calcd. for C₁₉H₁₅FN₆O, 362.1; m/z found 363.1 [M+H]⁺. ¹H NMR (400 MHz, DMSO-d₆): δ 13.53 (s, 1H), 8.45 (d, J=4.8 Hz, 1H), 8.24 (d, J=2.9 Hz, 1H), 7.90-7.85 (m, 1H), 7.81-7.75 (m, 1H), 7.29 (s, 1H), 7.04 (d, J=4.8 Hz, 1H), 4.86 (s, 2H), 4.34 (s, 2H), 1.08-1.04 (m, 2H), 0.89-0.84 (m, 2H).

### Example 433: (*R)-1′. 1′-Difluoro-2-(5-fluoro-2-pyridyl)-3-(1H-pyrazolo[3,4-b]pyridin-4-yl)spiro[4,7-dihydropyrazolo[5,1-c][1,4]oxazine-6,2′-cyclopropane]

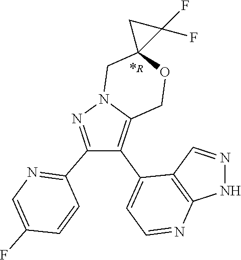

The title compound was prepared in a manner analogous to Example 1, Steps A-B, except using 3′-bromo-2,2-difluoro-2′-(5-fluoropyridin-2-yl)-4′H,7′H-spiro[cyclopropane-1,6′-pyrazolo[5,1-c][1,4]oxazine] (Intermediate 191) instead of 3-bromo-2-(5-fluoropyridin-2-yl)-6,7-dihydro-4H-pyrazolo[5,1-c][1,4]oxazine (Intermediate 37) and 4-(4,4,5,5-Tetramethyl-1,3,2-dioxaborolan-2-yl)-1-((2-(trimethylsilyl)ethoxy)methyl)-1H-pyrazolo[3,4-b]pyridine (Intermediate 23) instead of 4-(4,4,5,5-tetramethyl-1,3,2-dioxaborolan-2-yl)-1-((2-(trimethylsilyl)ethoxy)methyl)-1H-pyrrolo[2,3-b]pyridine (Intermediate 21) in Step A. Purification (chiral SFC (DAICEL CHIRALCEL OD 250 mm×30 mm, 10 μm (isocratic elution: EtOH (containing 0.1% of 25% aq. NH₃): supercritical CO₂, 35%: 65% to 35%: 65% (v/v))) afforded the title compound: MS (ESI): mass calcd. for C₁₉H₁₃F₃N₆O 398.1; m/z found 399.0 [M+H]⁺; ¹H NMR (400 MHz, DMSO-d₆): δ 13.54 (br s, 1H), 8.46 (d, J=4.8 Hz, 1H), 8.25 (d, J=2.8 Hz, 1H), 7.95-7.74 (m, 2H), 7.30 (s, 1H), 7.06 (d, J=4.8 Hz, 1H), 5.18-4.92 (m, 2H), 4.64-4.44 (m, 2H), 2.20-2.06 (m, 2H); and (*S)-1′,1′-Difluoro-2-(5-fluoro-2-pyridyl)-3-(1H-pyrazolo[3,4-b]pyridin-4-yl)spiro[4,7-dihydropyrazolo[5,1-c][1,4]oxazine-6,2′-cyclopropane] (Example 434).

### Example 434: (*S)-1′. 1′-Difluoro-2-(5-fluoro-2-pyridyl)-3-(1H-pyrazolo[3,4-b]pyridin-4-yl)spiro[4,7-dihydropyrazolo[5,1-c][1,4]oxazine-6,2′-cyclopropane]

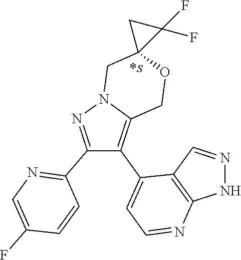

The title compound was isolated from chiral SFC purification of Example 433. MS (ESI): mass calcd. for C₁₉H₁₃F₃N₆O 398.1; m/z found 399.1 [M+H]⁺. ¹H NMR (400 MHz, DMSO-d₆): δ 13.53 (br s, 1H), 8.46 (d, J=4.8 Hz, 1H), 8.24 (d, J=2.8 Hz, 1H), 7.95-7.70 (m, 2H), 7.30 (s, 1H), 7.06 (d, J=4.8 Hz, 1H), 5.18-4.89 (m, 2H), 4.69-4.38 (m, 2H), 2.20-2.06 (m, 2H).

### Example 435: (*R)-1′. 1′-Difluoro-3-(5-fluoro-1H-pyrazolo[3,4-b]pyridin-4-yl)-2-(5-fluoro-2-pyridyl)spiro[4,7-dihydropyrazolo[5,1-c][1,4]oxazine-6,2′-cyclopropane]

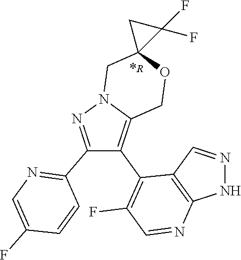

The title compound was prepared in a manner analogous to 4-[6,6-difluoro-2-(4-fluorophenyl)-5,7-dihydro-4H-pyrazolo[1,5-a]pyridin-3-yl]-6-methyl-1H-pyrazolo[3,4-d]pyrimidine (Example 279), except using 3-bromo-6,6-3′-bromo-2,2-difluoro-2′-(5-fluoropyridin-2-yl)-4′H,7′H-spiro[cyclopropane-1,6′-pyrazolo[5,1-c][1,4]oxazine] (Intermediate 191) instead of 3-bromo-6,6-difluoro-2-(4-fluorophenyl)-4,5,6,7-tetrahydropyrazolo[1,5-a]pyridine (Intermediate 52); 5-fluoro-4-iodo-1-(tetrahydro-2H-pyran-2-yl)-1H-pyrazolo[3,4-b]pyridine (Intermediate 230) instead of 4-chloro-6-methyl-1-((2-(trimethylsilyl)ethoxy)methyl)-1H-pyrazolo[3,4-d]pyrimidine (Intermediate 212); and using 4N HCl/dioxane as a deprotecting reagent instead of TFA/NH₃/MeOH. Purification (chiral SFC (DAICEL CHIRALCEL OD-H 250 mm×30 mm, 5 μm (isocratic elution: EtOH (containing 0.1% of 25% aq. NH₃): supercritical CO₂, 25%: 75% to 25%: 75% (v/v))) afforded the title compound: MS (ESI): mass calcd. for C₁₉H₁₂F₄N₆O 416.1; m/z found 417.0 [M+H]⁺; ¹H NMR (400 MHz, DMSO-d₆): δ 13.76 (d, J=12.4 Hz, 1H), 8.54 (d, J=4.8 Hz, 1H), 8.22 (br s, 1H), 8.03-7.91 (m, 1H), 7.86-7.75 (m, 1H), 7.74-7.50 (m, 1H), 5.09-4.75 (m, 2H), 4.70-4.44 (m, 2H), 2.23-2.09 (m, 2H); and (*S)-1′,1′-Difluoro-3-(5-fluoro-1H-pyrazolo[3,4-b]pyridin-4-yl)-2-(5-fluoro-2-pyridyl)spiro[4,7-dihydropyrazolo[5,1-c][1,4]oxazine-6,2′-cyclopropane] (Example 436).

### Example 436: (*S)-1′1l′-Difluoro-3-(5-fluoro-1H-pyrazolo[3,4-b]pyridin-4-yl)-2-(5-fluoro-2-pyridyl)spiro[4,7-dihydropyrazolo[5,1-c][1,4]oxazine-6,2′-cyclopropane]

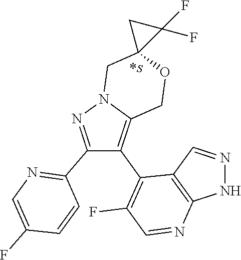

The title compound was isolated from chiral SFC purification of Example 435. MS (ESI): mass calcd. for C₁₉H₁₂F₄N₆O 416.1; m/z found 417.0 [M+H]⁺. ¹H NMR (400 MHz, DMSO-d₆): δ 13.76 (br s, 1H), 8.61-8.43 (m, 1H), 8.22 (br s, 1H), 8.04-7.92 (m, 1H), 7.85-7.54 (m, 2H), 5.07-4.78 (m, 2H), 4.69-4.47 (m, 2H), 2.16 (br s, 2H).

### Example 437: (*S)-3-(3-Chloro-1H-pyrazolo[3,4-b]pyridin-4-yl)-2-(5-fluoropyridin-2-yl)-6,6,7-trimethyl-6,7-dihydro-4H-pyrazolo[5,1-c][1,4]oxazine

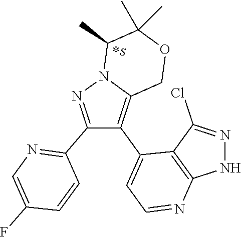

The title compound was prepared in a manner analogous to Example 224, except using (*S)-2-(5-fluoropyridin-2-yl)-6,6,7-trimethyl-3-(1H-pyrazolo[3,4-b]pyridin-4-yl)-6,7-dihydro-4H-pyrazolo[5,1-c][1,4]oxazine (Example 174) instead of 5-fluoro-4-(2-(5-fluoropyridin-2-yl)-5,6-dihydro-4H-pyrrolo[1,2-b]pyrazol-3-yl)-6-methyl-1H-pyrazolo[3,4-b]pyridine (Example 9). MS (ESI): mass calcd. for C₂₀H₁₈ClFN₆O 412.1; m/z found 413.2 [M+H]⁺. ¹H NMR (400 MHz, CD₃OD): δ 8.53 (t, J=4.9 Hz, 1H), 8.16-8.06 (m, 1H), 7.86-7.71 (m, 1H), 7.62-7.48 (m, 1H), 7.12 (dd, J=10.6, 4.7 Hz, 1H), 4.80 (d, J=16.0 Hz, 1H), 4.66 (dd, J=15.9, 14.0 Hz, 1H), 4.33-4.16 (m, 1H), 1.60 (dd, J=6.6, 2.0 Hz, 3H), 1.52-1.29 (m, 6H). N—H proton not observed.

### Example 438: 2-(4-Fluorophenyl)-3-(4-pyridyl)-6,7-dihydro-5H-pyrazolo[5,1-b][1,3]oxazine

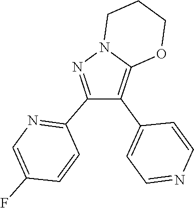 [SMILES: Fc1ccc(-c2nn3c(c2-c2ccncc2)OCCC3)nc1]

The title compound was prepared in a manner analogous to Example 1, Step A, except using pyridin-4-ylboronic acid instead of 4-(4,4,5,5-tetramethyl-1,3,2-dioxaborolan-2-yl)-1-((2-(trimethylsilyl)ethoxy)methyl)-1H-pyrrolo[2,3-b]pyridine (Intermediate 21) and 3-bromo-2-(4-fluorophenyl)-6,7-dihydro-5H-pyrazolo[5,1-b][1,3]oxazine (Intermediate 118) instead of 3-bromo-2-(5-fluoropyridin-2-yl)-6,7-dihydro-4H-pyrazolo[5,1-c][1,4]oxazine (Intermediate 37). MS (ESI): mass calcd. for C₁₇H₁₄FN₃O 295.1; m/z found 296.1 [M+H]⁺. ¹H NMR (400 MHz, DMSO-d₆): δ 8.42-8.39 (m, 2H), 7.42-7.37 (m, 2H), 7.25-7.19 (m, 2H), 7.13-7.10 (m, 2H), 4.46-4.40 (m, 2H), 4.18 (t, J=6.1 Hz, 2H), 2.31-2.24 (m, 2H).

### Example 439: N-(4-(2-(5-Fluoropyridin-2-yl)-6,7-dihydro-5H-pyrazolo[5,1-b][1,3]oxazin-3-yl)pyridin-2-yl)propionamide

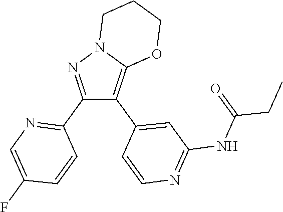 [SMILES: CCC(=O)Nc1cc(-c2c(-c3ccc(F)cn3)nn3c2OCCC3)ccn1]

The title compound was prepared in manner analogous to Example 223, except using 3-bromo-2-(5-fluoropyridin-2-yl)-6,7-dihydro-5H-pyrazolo[5,1-b][1,3]oxazine (Intermediate 119) instead of 3-bromo-2-(5-fluoropyridin-2-yl)-5,6-dihydro-4H-pyrrolo[1,2-b]pyrazole (Intermediate 39) and using N-(4-(4,4,5,5-tetramethyl-1,3,2-dioxaborolan-2-yl)pyridin-2-yl)propionamide instead of 1-ethyl-5-(4,4,5,5-tetramethyl-1,3,2-dioxaborolan-2-yl)-1H-pyrazolo[3,4-b]pyridine (Intermediate 31). Alternative purification employed: The crude reaction mixture was diluted with EtOAc/Hex and filtered through a pad of Celite® connected to a SiliaPrep SPE Thiol cartridge. The cartridge was washed with a mixture of EtOAc/DCM/MeOH (2:2:1) and the filtrate concentrated under reduced pressure. Purification (FCC, SiO₂, hexanes/(10% MeOH in EtOAc)) afforded the title compound. ¹H NMR (600 MHz, DMSO-d₆): δ 10.20 (s, 1H), 8.46 (dt, J=2.9, 0.6 Hz, 1H), 8.09 (dd, J=5.3, 0.8 Hz, 1H), 8.04-7.98 (m, 1H), 7.82-7.70 (m, 2H), 6.84 (dd, J=5.2, 1.6 Hz, 1H), 4.46-4.40 (m, 2H), 4.21 (t, J=6.1 Hz, 2H), 2.36-2.25 (m, 4H), 1.06-0.99 (m, 3H).

### Example 440: 2-(5-Fluoropyridin-2-yl)-3-(pyrazolo[1,5-a]pyridin-5-yl)-6,7-dihydro-5H-pyrazolo[5,1-b][1,3]oxazine

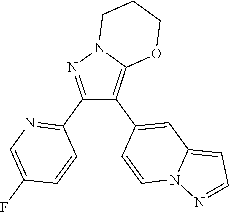 [SMILES: Fc1ccc(-c2nn3c(c2-c2ccn4nccc4c2)OCCC3)nc1]

The title compound was prepared in manner analogous to Example 223, except using 3-bromo-2-(5-fluoropyridin-2-yl)-6,7-dihydro-5H-pyrazolo[5,1-b][1,3]oxazine (Intermediate 119) instead of 3-bromo-2-(5-fluoropyridin-2-yl)-5,6-dihydro-4H-pyrrolo[1,2-b]pyrazole (Intermediate 39) and using pyrazolo[1,5-a]pyridin-5-ylboronic acid instead of 1-ethyl-5-(4,4,5,5-tetramethyl-1,3,2-dioxaborolan-2-yl)-1H-pyrazolo[3,4-b]pyridine (Intermediate 31). Alternative purification employed: The crude reaction mixture was diluted with EtOAc/Hex and filtered through a pad of Celite® connected to a SiliaPrep SPE Thiol cartridge. The cartridge was washed with a mixture of EtOAc/DCM/MeOH (2:2:1) and the filtrate concentrated under reduced pressure. Purification (FCC, SiO₂, hexanes/(10% MeOH in EtOAc)) afforded the title compound. ¹H NMR (600 MHz, DMSO-d₆): δ 8.51-8.45 (m, 2H), 7.92 (d, J=2.2 Hz, 1H), 7.84-7.75 (m, 2H), 7.52 (dd, J=2.0, 0.9 Hz, 1H), 4.46-4.42 (m, 2H), 4.23 (t, J=6.2 Hz, 2H), 2.30 (dt, J=10.4, 5.3 Hz, 2H

### Example 441: 3-(1-Ethyl-1H-pyrazolo[3,4-b]pyridin-5-yl)-2-(5-fluoropyridin-2-yl)-6,7-dihydro-5H-pyrazolo[5,1-b][1,3]oxazine

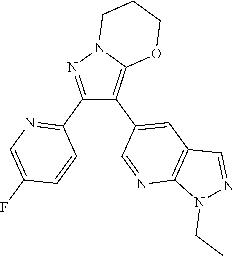 [SMILES: CCn1ncc2cc(-c3c(-c4ccc(F)cn4)nn4c3OCCC4)cnc21]

The title compound was prepared in manner analogous to Example 223, except using 3-bromo-2-(5-fluoropyridin-2-yl)-6,7-dihydro-5H-pyrazolo[5,1-b][1,3]oxazine (Intermediate 119) instead of 3-bromo-2-(5-fluoropyridin-2-yl)-5,6-dihydro-4H-pyrrolo[1,2-b]pyrazole (Intermediate 39). MS (ESI): mass calcd. for C₁₉H₁₇FN₆O 364.1; m/z found 365.1 [M+H]⁺. ¹H NMR (600 MHz, DMSO-d₆): δ 8.38 (dd, J=8.8, 2.5 Hz, 2H), 8.09 (s, 1H), 8.05 (d, J=2.0 Hz, 1H), 7.90-7.84 (m, 1H), 7.76 (td, J=8.8, 3.0 Hz, 1H), 4.48 (q, J=7.2 Hz, 2H), 4.42-4.38 (m, 2H), 4.24 (t, J=6.1 Hz, 2H), 2.31 (q, J=5.5 Hz, 3H), 1.44 (t, J=7.2 Hz, 3H).

### Example 442: N-(4-(2-(5-Fluoropyridin-2-yl)-6-methyl-6,7-dihydro-5H-pyrazolo[5,1-b][1,3]oxazin-3-yl)pyridin-2-yl)propionamide

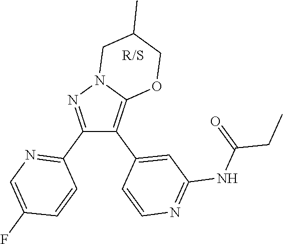 [SMILES: CCC(=O)Nc1cc(-c2c(-c3ccc(F)cn3)nn3c2OCC(C)C3)ccn1]

The title compound was prepared in manner analogous to Example 223, except using 3-bromo-2-(5-fluoropyridin-2-yl)-6-methyl-6,7-dihydro-5H-pyrazolo[5,1-b][1,3]oxazine (Intermediate 122) instead of 3-bromo-2-(5-fluoropyridin-2-yl)-5,6-dihydro-4H-pyrrolo[1,2-b]pyrazole (Intermediate 39) and using N-(4-(4,4,5,5-tetramethyl-1,3,2-dioxaborolan-2-yl)pyridin-2-yl)propionamide instead of 1-ethyl-5-(4,4,5,5-tetramethyl-1,3,2-dioxaborolan-2-yl)-1H-pyrazolo[3,4-b]pyridine (Intermediate 31). Alternative purification employed: The crude reaction mixture was diluted with EtOAc/Hex and filtered through a pad of Celite® connected to a SiliaPrep SPE Thiol cartridge. The cartridge was washed with a mixture of EtOAc/DCM/MeOH (2:2:1) and the filtrate concentrated under reduced pressure. Purification (FCC, SiO₂, hexanes/(10% MeOH in EtOAc)) afforded the title compound. MS (ESI): mass calcd. for C₂₀H₂₀FN₅O₂ 381.2; m/z found 382.1 [M+H]⁺. ¹H NMR (600 MHz, DMSO-d₆): δ 10.20 (s, 1H), 8.47 (dt, J=2.9, 0.6 Hz, 1H), 8.09 (dd, J=5.2, 0.8 Hz, 1H), 8.03 (s, 1H), 7.81-7.70 (m, 2H), 6.85 (dd, J=5.2, 1.6 Hz, 1H), 4.45 (dd, J=10.5, 3.4 Hz, 1H), 4.30 (dd, J=12.1, 5.2 Hz, 1H), 4.08 (dd, J=10.7, 9.3 Hz, 1H), 3.84 (dd, J=12.0, 8.9 Hz, 1H), 2.34 (q, J=7.5 Hz, 2H), 1.24 (s, 1H), 1.09 (d, J=6.8 Hz, 3H), 1.03 (t, J=7.5 Hz, 3H).

### Example 443: 2-(5-Fluoropyridin-2-yl)-6-methyl-3-(pyrazolo[1,5-a]pyridin-5-yl)-6,7-dihydro-5H-pyrazolo[5,1-b][1,3]oxazine

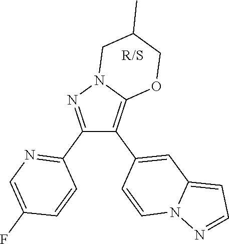 [SMILES: CC1COc2c(-c3ccn4nccc4c3)c(-c3ccc(F)cn3)nn2C1]

The title compound was prepared in manner analogous to Example 223, except using 3-bromo-2-(5-fluoropyridin-2-yl)-6-methyl-6,7-dihydro-5H-pyrazolo[5,1-b][1,3]oxazine (Intermediate 122) instead of 3-bromo-2-(5-fluoropyridin-2-yl)-5,6-dihydro-4H-pyrrolo[1,2-b]pyrazole (Intermediate 39) and using pyrazolo[1,5-a]pyridin-5-ylboronic acid instead of 1-ethyl-5-(4,4,5,5-tetramethyl-1,3,2-dioxaborolan-2-yl)-1H-pyrazolo[3,4-b]pyridine (Intermediate 31).

Alternative purification employed: The crude reaction mixture was diluted with EtOAc/Hex and filtered through a pad of Celite® connected to a SiliaPrep SPE Thiol cartridge. The cartridge was washed with a mixture of EtOAc/DCM/MeOH (2:2:1) and the filtrate concentrated under reduced pressure. Purification (FCC, SiO₂, hexanes/(10% MeOH in EtOAc)) afforded the title compound. MS (ESI): mass calcd. for C₁₉H₁₆FN₅O 349.1; m/z found 350.1 [M+H]⁺. ¹H NMR (600 MHz, DMSO-d₆) δ 8.53-8.45 (m, 2H), 7.93 (d, J=2.3 Hz, 1H), 7.87-7.73 (m, 2H), 7.52 (dd, J=1.9, 1.0 Hz, 1H), 6.66-6.58 (m, 1H), 6.52 (dd, J=2.2, 0.9 Hz, 1H), 4.52-4.40 (m, 1H), 4.37-4.26 (m, 1H), 4.09 (dd, J=10.7, 9.2 Hz, 1H), 3.85 (dd, J=12.0, 8.8 Hz, 1H), 2.04-1.96 (m, 1H), 1.10 (d, J=6.8 Hz, 3H).

### Example 444: 3-(1-Ethyl-1H-pyrazolo[3,4-b]pyridin-5-yl)-2-(5-fluoropyridin-2-yl)-6-methyl-6,7-dihydro-5H-pyrazolo[5,1-b][1,3]oxazine

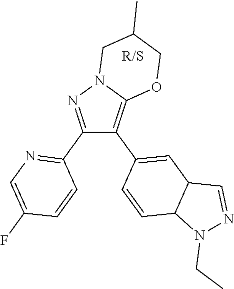 [SMILES: CCN1N=CC2C=C(c3c(-c4ccc(F)cn4)nn4c3OCC(C)C4)C=CC21]

The title compound was prepared in manner analogous to Example 223, except using 3-bromo-2-(5-fluoropyridin-2-yl)-6-methyl-6,7-dihydro-5H-pyrazolo[5,1-b][1,3]oxazine (Intermediate 122) instead of 3-bromo-2-(5-fluoropyridin-2-yl)-5,6-dihydro-4H-pyrrolo[1,2-b]pyrazole (Intermediate 39). MS (ESI): mass calcd. for C₂₀H₁₉FN₆O 378.2; m/z found 379.2 [M+H]⁺. ¹H NMR (600 MHz, DMSO-d₆): δ 8.38 (d, J=2.0 Hz, 1H), 8.37 (dt, J=3.0, 0.6 Hz, 1H), 8.09 (s, 1H), 8.05 (d, J=2.0 Hz, 1H), 7.86 (ddd, J=8.9, 4.7, 0.6 Hz, 1H), 7.75 (td, J=8.8, 3.0 Hz, 1H), 4.47 (q, J=7.2 Hz, 2H), 4.39 (dd, J=10.7, 3.4 Hz, 1H), 4.32 (dd, J=12.2, 5.2 Hz, 1H), 4.04 (dd, J=10.7, 9.2 Hz, 1H), 3.85 (dd, J=12.0, 8.9 Hz, 1H), 2.53-2.51 (m, 1H), 1.43 (t, J=7.2 Hz, 3H), 1.09 (d, J=6.8 Hz, 3H).

### Example 445: N-(4-(2-(5-Fluoropyridin-2-yl)-7-methyl-6,7-dihydro-5H-pyrazolo[5,1-b][1,3]oxazin-3-yl)pyridin-2-yl)propionamide

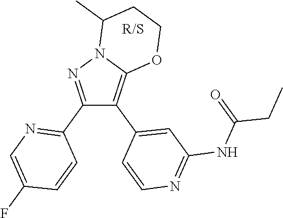 [SMILES: CCC(=O)Nc1cc(-c2c(-c3ccc(F)cn3)nn3c2OCCC3C)ccn1]

The title compound was prepared in manner analogous to Example 223, except using 3-bromo-2-(5-fluoropyridin-2-yl)-7-methyl-6,7-dihydro-5H-pyrazolo[5,1-b][1,3]oxazine (Intermediate 192) instead of 3-bromo-2-(5-fluoropyridin-2-yl)-5,6-dihydro-4H-pyrrolo[1,2-b]pyrazole (Intermediate 39) and using N-(4-(4,4,5,5-tetramethyl-1,3,2-dioxaborolan-2-yl)pyridin-2-yl)propionamide instead of 1-ethyl-5-(4,4,5,5-tetramethyl-1,3,2-dioxaborolan-2-yl)-1H-pyrazolo[3,4-b]pyridine (Intermediate 31). Alternative purification employed: The crude reaction mixture was diluted with EtOAc/Hex and filtered through a pad of Celite® connected to a SiliaPrep SPE Thiol cartridge. The cartridge was washed with a mixture of EtOAc/DCM/MeOH (2:2:1) and the filtrate concentrated under reduced pressure. The crude oil was purified by FCC (SiO₂, hex/(10% MeOH in EtOAc)). MS (ESI): mass calcd. for C₂₀H₂₀FN₅O₂ 381.2; m/z found 381.6 [M+H]⁺. ¹H NMR (600 MHz, DMSO-d₆): δ 10.19 (s, 1H), 8.46 (dt, J=2.9, 0.6 Hz, 1H), 8.08 (dd, J=5.3, 0.8 Hz, 1H), 8.01 (s, 1H), 7.84-7.68 (m, 2H), 6.82 (dd, J=5.2, 1.6 Hz, 1H), 4.50 (ddd, J=9.9, 6.3, 3.2 Hz, 1H), 4.46-4.34 (m, 2H), 2.45-2.37 (m, 1H), 2.33 (q, J=7.5 Hz, 2H), 2.08-2.00 (m, 1H), 1.55 (d, J=6.5 Hz, 3H), 1.02 (t, J=7.6 Hz, 3H).

### Example 446: 2-(5-Fluoropyridin-2-yl)-7-methyl-3-(pyrazolo[1,5-a]pyridin-5-yl)-6,7-dihydro-5H-pyrazolo[5,1-b][1,3]oxazine

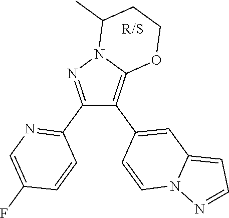 [SMILES: CC1CCOc2c(-c3ccn4nccc4c3)c(-c3ccc(F)cn3)nn21]

The title compound was prepared in manner analogous to Example 223, except using 3-bromo-2-(5-fluoropyridin-2-yl)-7-methyl-6,7-dihydro-5H-pyrazolo[5,1-b][1,3]oxazine (Intermediate 192) instead of 3-bromo-2-(5-fluoropyridin-2-yl)-5,6-dihydro-4H-pyrrolo[1,2-b]pyrazole (Intermediate 39) and using pyrazolo[1,5-a]pyridin-5-ylboronic acid instead of 1-ethyl-5-(4,4,5,5-tetramethyl-1,3,2-dioxaborolan-2-yl)-1H-pyrazolo[3,4-b]pyridine (Intermediate 31). Alternative purification employed: The crude reaction mixture was diluted with EtOAc/Hex and filtered through a pad of Celite® connected to a SiliaPrep SPE Thiol cartridge. The cartridge was washed with a mixture of EtOAc/DCM/MeOH (2:2:1) and the filtrate concentrated under reduced pressure. Purification (FCC, SiO₂, hexanes/(10% MeOH in EtOAc)) afforded the title compound. MS (ESI): mass calcd. for C₁₉H₁₆FN₅O 349.1; m/z found 349.6 [M+H]⁺. ¹H NMR (600 MHz, DMSO-d₆): δ 8.51-8.45 (m, 2H), 7.92 (d, J=2.2 Hz, 1H), 7.85-7.75 (m, 2H), 7.51 (dd, J=1.9, 0.9 Hz, 1H), 6.61 (dd, J=7.2, 1.9 Hz, 1H), 6.51 (dd, J=2.2, 0.9 Hz, 1H), 4.54-4.48 (m, 1H), 4.47-4.37 (m, 2H), 2.46-2.40 (m, 1H), 2.09-2.00 (m, 1H), 1.58 (d, J=6.5 Hz, 3H).

### Example 447: N-(4-(2-(5-Fluoropyridin-2-yl)-6,6-dimethyl-6,7-dihydro-5H-pyrazolo[5,1-b][1,3]oxazin-3-yl)pyridin-2-yl)propionamide

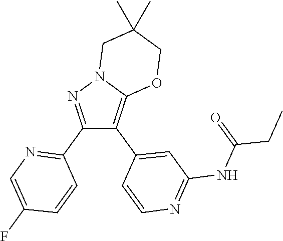 [SMILES: CCC(=O)Nc1cc(-c2c(-c3ccc(F)cn3)nn3c2OCC(C)(C)C3)ccn1]

The title compound was prepared in manner analogous to Example 223, except using 3-bromo-2-(5-fluoropyridin-2-yl)-6,6-dimethyl-6,7-dihydro-5H-pyrazolo[5,1-b][1,3]oxazine (Intermediate 128) instead of 3-bromo-2-(5-fluoropyridin-2-yl)-5,6-dihydro-4H-pyrrolo[1,2-b]pyrazole (Intermediate 39) and using N-(4-(4,4,5,5-tetramethyl-1,3,2-dioxaborolan-2-yl)pyridin-2-yl)propionamide instead of 1-ethyl-5-(4,4,5,5-tetramethyl-1,3,2-dioxaborolan-2-yl)-1H-pyrazolo[3,4-b]pyridine (Intermediate 31). Alternative purification employed: The crude reaction mixture was diluted with EtOAc/Hex and filtered through a pad of Celite® connected to a SiliaPrep SPE Thiol cartridge. The cartridge was washed with a mixture of EtOAc/DCM/MeOH (2:2:1) and the filtrate concentrated under reduced pressure. Purification (FCC, SiO₂, hexanes/(10% MeOH in EtOAc)) afforded the title compound. ¹H NMR (600 MHz, DMSO-d₆): δ 10.21 (s, 1H), 8.47 (d, J=2.9 Hz, 1H), 8.11 (dd, J=5.3, 0.8 Hz, 1H), 8.03 (s, 1H), 7.83-7.72 (m, 2H), 6.88 (dd, J=5.2, 1.6 Hz, 1H), 4.13 (s, 2H), 3.97 (s, 2H), 2.34 (q, J=7.5 Hz, 2H), 1.12 (s, 6H), 1.03 (t, J=7.5 Hz, 3H).

### Example 448: 2-(4-Fluorophenyl)-6,6-dimethyl-3-(1H-pyrrolo[3,2-b]pyridin-7-yl)-5,7-dihydropyrazolo[5,1-b][1,3]oxazine

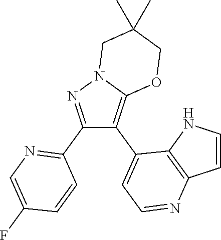 [SMILES: CC1(C)COc2c(-c3ccnc4cc[nH]c34)c(-c3ccc(F)cn3)nn2C1]

Step A. 2-(4-Fluorophenyl)-6,6-dimethyl-3-(4,4,5,5-tetramethyl-1,3,2-dioxaborolan-2-yl)-6,7-dihydro-5H-pyrazolo[5,1-b][1,3]oxazine. i-PrMgCl·LiCl (3.85 mL, 5.01 mmol) was added dropwise to a 0° C. solution consisting of 3-bromo-2-(4-fluorophenyl)-6,6-dimethyl-6,7-dihydro-5H-pyrazolo[5,1-b][1,3]oxazine (Intermediate 194, 810 mg, 2.49 mmol) and THF (8 mL) under N₂. The mixture was warmed to room-temperature and then stirred at 50° C. for 3 hours. The reaction mixture was then cooled to 0° C. and 2-isopropoxy-4,4,5,5-tetramethyl-1,3,2-dioxaborolane (510 mg, 2.74 mmol) dissolved in THF (2 mL) were added dropwise. The mixture was warmed to room-temperature and stirred at this temperature for 16 hours. The reaction mixture was quenched with sat. NH₄Cl (20 mL) and extracted with ethyl acetate (15 mL×3). The combined organic extracts were washed with brine (10 mL×2), dried over anhydrous Na₂SO₄, filtered, and concentrated to dryness under reduced pressure Purification (FCC, SiO₂, petroleum ether:ethyl acetate=1:0 to 2:1) afforded the title compound (400 mg, 42%) as a yellow solid. MS (ESI): mass calcd. for C₂₀H₂₆BFN₂O₃ 372.2; m/z found 373.1 [M+H]⁺.

Step B. 2-(4-Fluorophenyl)-6,6-dimethyl-3-(1H-pyrrolo[3,2-b]pyridin-7-yl)-6,7-dihydro-5H-pyrazolo[5,1-b][1,3]oxazine. 7-Bromo-1H-pyrrolo[3,2-b]pyridine (110 mg, 0.558 mmol), 2-(4-fluorophenyl)-6,6-dimethyl-3-(4,4,5,5-tetramethyl-1,3,2-dioxaborolan-2-yl)-6,7-dihydro-5H-pyrazolo[5,1-b][1,3]oxazine (240 mg, 0.645 mmol), and Cs₂CO₃ (530 mg, 1.63 mmol) were dissolved in 1,4-dioxane (6 mL) and H₂O (1.5 mL). The resultant mixture was sparged with N₂ for 5 minutes and then treated with Pd(PPh₃)₄ (62 mg, 0.054 mmol). The mixture was sparged with N₂ for another 5 minutes and heated at 100° C. for 16 hours. The suspension was filtered through a pad of Celite® and concentrated under reduced pressure. Purification (preparative HPLC using a Xtimate C18 150×40 mm×5 μm column (eluent: 31% to 61% (v/v) CH₃CN and H₂O with 0.05% NH₃)) followed by further purification (preparative HPLC purification using a Boston Green ODS 150×30 mm×5 μm column (eluent: 20% to 50% (v/v) CH₃CN and H₂O with 0.225% HCOOH)) afforded the title compound (33.5 mg, 17%) as a white solid. MS (ESI): mass calcd. for C₂₁H₁₉FN₄O 362.2; m/z found 363.1 [M+H]⁺. ¹H NMR (400 MHz, DMSO-d₆): δ 10.94 (s, 1H), 8.26-8.13 (m, 1H), 7.55-7.51 (m, 1H), 7.37-7.31 (m, 2H), 7.10-7.03 (m, 2H), 6.79-6.73 (m, 1H), 6.56 (s, 1H), 4.03 (s, 2H), 3.96 (s, 2H), 1.12 (s, 6H).

### Example 449: 2-(5-Fluoropyridin-2-yl)-6,6-dimethyl-3-(pyrazolo[1,5-a]pyridin-5-yl)-6,7-dihydro-5H-pyrazolo[5,1-b][1,3]oxazine

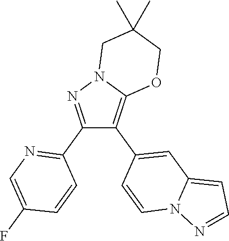 [SMILES: CC1(C)COc2c(-c3ccn4nccc4c3)c(-c3ccc(F)cn3)nn2C1]

The title compound was prepared in manner analogous to Example 223, except using 3-bromo-2-(5-fluoropyridin-2-yl)-6,6-dimethyl-6,7-dihydro-5H-pyrazolo[5,1-b][1,3]oxazine (Intermediate 128) instead of 3-bromo-2-(5-fluoropyridin-2-yl)-5,6-dihydro-4H-pyrrolo[1,2-b]pyrazole (Intermediate 39) and using pyrazolo[1,5-a]pyridin-5-ylboronic acid instead of 1-ethyl-5-(4,4,5,5-tetramethyl-1,3,2-dioxaborolan-2-yl)-1H-pyrazolo[3,4-b]pyridine (Intermediate 31).

Alternative purification employed: The reaction mixture was diluted with EtOAc/Hex and filtered through a pad of Celite® connected to a SiliaPrep SPE Thiol cartridge. The cartridge was washed with a mixture of EtOAc/DCM/MeOH (2:2:1) and the filtrate concentrated under reduced pressure. Purification (FCC, SiO₂, hexanes/(10% MeOH in EtOAc)) afforded the title compound. ¹H NMR (600 MHz, DMSO-d₆): δ 8.50 (dt, J=7.3, 1.0 Hz, 1H), 8.48 (dt, J=3.0, 0.6 Hz, 1H), 7.93 (d, J=2.2 Hz, 1H), 7.84-7.76 (m, 2H), 7.54 (dd, J=2.0, 0.9 Hz, 1H), 6.65-6.62 (m, 1H), 6.53 (dd, J=2.3, 0.9 Hz, 1H), 4.12 (s, 2H), 3.98 (s, 2H), 1.14 (s, 6H).

### Example 450: 2-(5-Fluoro-2-pyridyl)-6,6-dimethyl-3-(1H-pyrrolo[3,2-b]pyridin-7-yl)-5,7-dihydropyrazolo[5,1-b][1,3]oxazine

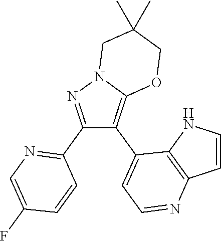 [SMILES: CC1(C)COc2c(-c3ccnc4cc[nH]c34)c(-c3ccc(F)cn3)nn2C1]

The title compound was prepared in a manner analogous to Example 279, except using 3-bromo-2-(5-fluoro-2-pyridyl)-6,6-dimethyl-5,7-dihydropyrazolo[5,1-b][1,3]oxazine (Intermediate 128) instead of 3-bromo-6,6-difluoro-2-(4-fluorophenyl)-4,5,6,7-tetrahydropyrazolo[1,5-a]pyridine (Intermediate 52) and 7-bromo-1H-pyrrolo[3,2-b]pyridine instead of 4-chloro-6-methyl-1-((2-(trimethylsilyl)ethoxy)methyl)-1H-pyrazolo[3,4-d]pyrimidine (Intermediate 212). MS (ESI): mass calcd. for C₂₀H₁₈FN₅O 363.1; m/z found 364.1 [M+H]⁺. ¹H NMR (400 MHz, DMSO-d₆): δ 10.76 (br s, 1H), 8.22 (d, J=2.9 Hz, 1H), 8.14 (d, J=4.9 Hz, 1H), 7.83-7.75 (m, 1H), 7.68 (dt, J=3.0, 8.8 Hz, 1H), 7.45 (t, J=3.0 Hz, 1H), 6.75 (d, J=4.9 Hz, 1H), 6.48 (d, J=2.2 Hz, 1H), 4.06-3.91 (m, 4H), 1.09 (s, 6H).

### Example 451: 2-(5-Fluoro-2-pyridyl)-6,6-dimethyl-3-(6-methylpyrazolo[1,5-a]pyridin-5-yl)-5,7-dihydropyrazolo[5,1-b][1,3]oxazine

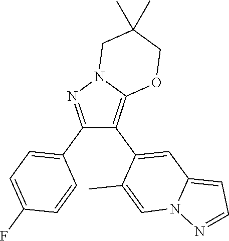 [SMILES: Cc1cn2nccc2cc1-c1c(-c2ccc(F)cc2)nn2c1OCC(C)(C)C2]

The title compound was prepared in a manner analogous to Example 1, Step A, except using 6-methyl-5-(4,4,5,5-tetramethyl-1,3,2-dioxaborolan-2-yl)pyrazolo[1,5-a]pyridine (Intermediate 225) instead of 4-(4,4,5,5-tetramethyl-1,3,2-dioxaborolan-2-yl)-1-((2-(trimethylsilyl)ethoxy)methyl)-1H-pyrrolo[2,3-b]pyridine (Intermediate 21) and 3-bromo-2-(5-fluoro-2-pyridyl)-6,6-dimethyl-5,7-dihydropyrazolo[5,1-b][1,3]oxazine (Intermediate 128) instead of 3-bromo-2-(5-fluoropyridin-2-yl)-6,7-dihydro-4H-pyrazolo[5,1-c][1,4]oxazine (Intermediate 37). MS (ESI): mass calcd. for C₂₁H₂₀FN₅O 377.2; m/z found 378.1 [M+H]⁺. ¹H NMR (400 MHz, DMSO-d₆): δ 8.49 (s, 1H), 8.29 (d, J=2.9 Hz, 1H), 7.87-7.80 (m, 2H), 7.72-7.65 (m, 1H), 7.35 (s, 1H), 6.44 (s, 1H), 3.97 (s, 4H), 1.89 (s, 3H), 1.08 (s, 6H).

### Example 452: 3-(1-Ethyl-1H-pyrazolo[3,4-b]pyridin-5-yl)-2-(5-fluoropyridin-2-yl)-6,6-dimethyl-6,7-dihydro-5H-pyrazolo[5,1-b][1,3]oxazine

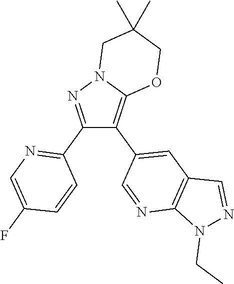 [SMILES: CCn1ncc2cc(-c3c(-c4ccc(F)cn4)nn4c3OCC(C)(C)C4)cnc21]

The title compound was prepared in manner analogous to Example 223, except using 3-bromo-2-(5-fluoropyridin-2-yl)-6,6-dimethyl-6,7-dihydro-5H-pyrazolo[5,1-b][1,3]oxazine (Intermediate 128) instead of 3-bromo-2-(5-fluoropyridin-2-yl)-5,6-dihydro-4H-pyrrolo[1,2-b]pyrazole (Intermediate 39). MS (ESI): mass calcd. for C₂₁H₂₁FN₆O 392.2; m/z found 393.2 [M+H]⁺. ¹H NMR (600 MHz, DMSO-d₆): δ 8.40 (d, J=2.0 Hz, 1H), 8.38 (d, J=3.0 Hz, 1H), 8.10 (s, 1H), 8.07 (d, J=2.1 Hz, 1H), 7.90-7.85 (m, 1H), 7.76 (td, J=8.8, 3.0 Hz, 1H), 4.48 (q, J=7.2 Hz, 2H), 4.08 (s, 2H), 3.99 (s, 2H), 1.44 (t, J=7.2 Hz, 3H), 1.14 (s, 6H).

### Example 453: 2-(4-Fluorophenyl)-6,6-dimethyl-3-(1H-pyrazolo[4,3-b]pyridin-7-yl)-5,7-dihydropyrazolo[5,1-b][1,3]oxazine

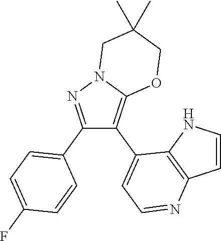 [SMILES: CC1(C)COc2c(-c3ccnc4cc[nH]c34)c(-c3ccc(F)cc3)nn2C1]

The title compound was prepared in a manner analogous to Example 294, except using 3-bromo-2-(4-fluorophenyl)-6,6-dimethyl-6,7-dihydro-5H-pyrazolo[5,1-b][1,3]oxazine (Intermediate 194) instead of 3-bromo-6-(fluoromethyl-d₂)-2-(5-fluoropyridin-2-yl)-6-(methyl-d₃)-4,5,6,7-tetrahydropyrazolo[1,5-a]pyridine (Intermediate 169); 7-chloro-1-((2-(trimethylsilyl)ethoxy)methyl)-1H-pyrazolo[4,3-b]pyridine (Intermediate 191) instead of 5-fluoro-4-iodo-1-(tetrahydro-2H-pyran-2-yl)-1H-pyrazolo[3,4-b]pyridine (Intermediate 230); and CatacXium A-Pd-G2 instead of Pd(tBu₃P)₂. MS (ESI): mass calcd. for C₂₀H₁₈FN₅O 363.1; m/z found 364.1 [M+H]⁺. ¹H NMR (400 MHz, DMSO-d₆): δ 13.05 (s, 1H), 8.40 (d, J=4.5 Hz, 1H), 8.28 (d, J=1.3 Hz, 1H), 7.38-7.32 (m, 2H), 7.13-7.07 (m, 2H), 6.97 (d, J=4.5 Hz, 1H), 4.08 (s, 2H), 3.97 (s, 2H), 1.12 (s, 6H).

### Example 454: 2-(5-Fluoro-2-pyridyl)-6,6-dimethyl-3-(1H-pyrazolo[4,3-b]pyridin-7-yl)-5,7-dihydropyrazolo[5,1-b][1,3]oxazine

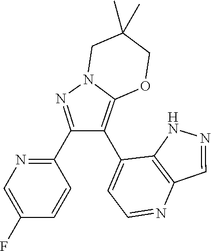 [SMILES: CC1(C)COc2c(-c3ccnc4cn[nH]c34)c(-c3ccc(F)cn3)nn2C1]

The title compound was prepared in a manner analogous to Example 294, except using 3-bromo-2-(5-fluoro-2-pyridyl)-6,6-dimethyl-5,7-dihydropyrazolo[5,1-b][1,3]oxazine (Intermediate 128) instead of 3-bromo-6-(fluoromethyl-d₂)-2-(5-fluoropyridin-2-yl)-6-(methyl-d₃)-4,5,6,7-tetrahydropyrazolo[1,5-a]pyridine (Intermediate 169); 7-chloro-1-((2-(trimethylsilyl)ethoxy)methyl)-1H-pyrazolo[4,3-b]pyridine (Intermediate 191) instead of 5-fluoro-4-iodo-1-(tetrahydro-2H-pyran-2-yl)-1H-pyrazolo[3,4-b]pyridine (Intermediate 230); and CatacXium A-Pd-G2 instead of Pd(tBu₃P)₂. MS (ESI): mass calcd. for C₁₉H₁₇FN₆O 364.1; m/z found 492.2 [M+H]⁺. ¹H NMR (400 MHz, DMSO-d₆): δ 12.87 (s, 1H), 8.38 (s, 1H), 8.28-8.15 (m, 2H), 7.95-7.86 (m, 1H), 7.80-7.69 (m, 1H), 7.07 (s, 1H), 4.09 (s, 2H), 4.00 (s, 2H), 1.12 (s, 6H).

### Example 455: N-(4-(2′-(4-Fluorophenyl)-5′H,7′H-spiro[cyclopropane-1,6′-pyrazolo[5,1-b][1,3]oxazin]-3′-yl)pyridin-2-yl)propionamide

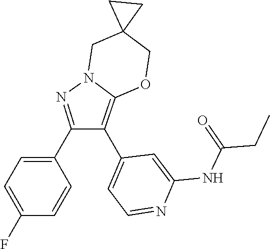 [SMILES: CCC(=O)Nc1cc(-c2c(-c3ccc(F)cc3)nn3c2OCC2(CC2)C3)ccn1]

The title compound was prepared in a manner analogous to Example 1, Step A, except using 3′-bromo-2′-(4-fluorophenyl)-5′H,7′H-spiro[cyclopropane-1,6′-pyrazolo[5,1-b][1,3]oxazine](Intermediate 195) instead of 3-bromo-2-(5-fluoropyridin-2-yl)-6,7-dihydro-4H-pyrazolo[5,1-c][1,4]oxazine (Intermediate 37); and N-[4-(4,4,5,5-tetramethyl-[1,3,2]dioxaborolan-2-yl)-pyridin-2-yl]-propionamide instead of 4-(4,4,5,5-tetramethyl-1,3,2-dioxaborolan-2-yl)-1-((2-(trimethylsilyl)ethoxy)methyl)-1H-pyrrolo[2,3-b]pyridine (Intermediate 21). MS (ESI): mass calcd. for C₂₂H₂₁FN₄O₂ 392.2; m/z found 393.1 [M+H]⁺. ¹H NMR (400 MHz, CD₃OD): δ 8.15-7.93 (m, 2H), 7.53-7.34 (m, 2H), 7.17-7.02 (m, 2H), 6.86 (dd, J=5.3, 1.6 Hz, 1H), 4.23 (s, 2H), 4.08 (s, 2H), 2.40 (q, J=7.6 Hz, 2H), 1.17 (t, J=7.6 Hz, 3H), 1.03-0.75 (m, 4H). N—H proton not observed.

### Example 456: N-(4-(2′-(5-Fluoropyridin-2-yl)-5′H,7′H-spiro[cyclopropane-1,6′-pyrazolo[5,1-b][1,3]oxazin]-3′-yl)pyridin-2-yl)cyclopropanecarboxamide

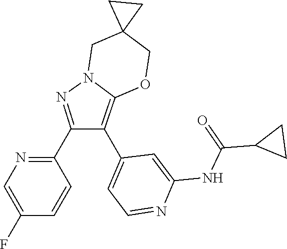 [SMILES: O=C(Nc1cc(-c2c(-c3ccc(F)cn3)nn3c2OCC2(CC2)C3)ccn1)C1CC1]

The title compound was prepared in manner analogous to Example 223, except using 2′-(5-fluoropyridin-2-yl)-5′H,7′H-spiro[cyclopropane-1,6′-pyrazolo[5,1-b][1,3]oxazine] (Intermediate 196) instead of 3-bromo-2-(5-fluoropyridin-2-yl)-5,6-dihydro-4H-pyrrolo[1,2-b]pyrazole (Intermediate 39); and using N-(4-(4,4,5,5-tetramethyl-1,3,2-dioxaborolan-2-yl)pyridin-2-yl)cyclopropanecarboxamide instead of 1-ethyl-5-(4,4,5,5-tetramethyl-1,3,2-dioxaborolan-2-yl)-1H-pyrazolo[3,4-b]pyridine (Intermediate 31). Alternative purification employed: The crude reaction mixture was diluted with DCM and filtered through a SiliaPrep SPE Thiol cartridge.

The cartridge was washed with EtOAc/DCM and MeOH and the filtrate concentrated under reduced pressure. Purification (Basic AQQU Prep with ACN/NH₄OH 0-100% over 25 min. and then purified on the acidic AQQU prep (0.05% TFA/ACN)/(0.05% TFA in H₂O) 0-100% over 30 min.) then concentrated and filtered through a silicycle Bicarb filter to get the free base; afforded the title compound. MS (ESI): mass calcd. for C₂₂H₂₀FN₅O₂ 405.2; m/z found 406.1 [M+H]⁺. ¹H NMR (600 MHz, DMSO-d₆): δ ¹H NMR (400 MHz, DMSO-d₆) δ 10.56 (s, 1H), 8.45 (d, J=2.8 Hz, 1H), 8.10 (dd, J=5.3, 0.8 Hz, 1H), 8.01 (t, J=1.1 Hz, 1H), 7.91-7.66 (m, 2H), 6.84 (dd, J=5.3, 1.6 Hz, 1H), 4.22 (s, 2H), 4.10 (s, 2H), 1.96 (p, J=6.4 Hz, 1H), 0.83 (s, 4H), 0.78-0.72 (m, 4H).

### Example 457: 2′-(4-Fluorophenyl)-3′-(1H-pyrazolo[3,4-b]pyridin-4-yl)-5′H,7′H-spiro[cyclopropane-1,6′-pyrazolo[5,1-b][1,3]oxazine]

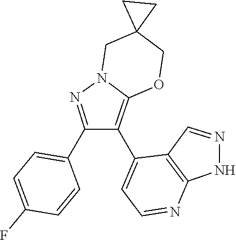 [SMILES: Fc1ccc(-c2nn3c(c2-c2ccnc4[nH]ncc24)OCC2(CC2)C3)cc1]

The title compound was prepared in a manner analogous to Example 1, Steps A-B, except using 3′-bromo-2′-(4-fluorophenyl)-5′H,7′H-spiro[cyclopropane-1,6′-pyrazolo[5,1-b][1,3]oxazine](Intermediate 195) instead of 3-bromo-2-(5-fluoropyridin-2-yl)-6,7-dihydro-4H-pyrazolo[5,1-c][1,4]oxazine (Intermediate 37); and trimethyl-[2-[[4-(4,4,5,5-tetramethyl-1,3,2-dioxaborolan-2-yl)pyrazolo[3,4-b]pyridin-1-yl]methoxy]ethyl]silane (Intermediate 23) instead of 4-(4,4,5,5-tetramethyl-1,3,2-dioxaborolan-2-yl)-1-((2-(trimethylsilyl)ethoxy)methyl)-1H-pyrrolo[2,3-b]pyridine (Intermediate 21) in Step A. MS (ESI): mass calcd. for C₂₀H₁₆FN₅O 361.1; m/z found 362.1 [M+H]⁺. ¹H NMR (500 MHz, CD₃OD) δ 8.36 (d, J=5.0 Hz, 1H), 7.56 (s, 1H), 7.45-7.33 (m, 2H), 7.12-6.93 (m, 3H), 4.24 (s, 2H), 4.14 (s, 2H), 0.92 (dt, J=6.8, 2.0 Hz, 4H). N—H proton not observed.

### Example 458: 3′-(3-Chloro-1H-pyrazolo[3,4-b]pyridin-4-yl)-2′-(5-fluoropyridin-2-yl)-5′H,7′H-spiro[cyclopropane-1,6′-pyrazolo[5,1-b][1,3]oxazine]

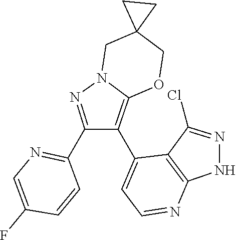 [SMILES: Fc1ccc(-c2nn3c(c2-c2ccnc4[nH]nc(Cl)c24)OCC2(CC2)C3)nc1]

The title compound was prepared in a manner analogous to Example 224, except using 2′-(5-fluoropyridin-2-yl)-3′-(1H-pyrazolo[3,4-b]pyridin-4-yl)-5′H,7′H-spiro[cyclopropane-1,6′-pyrazolo[5,1-b][1,3]oxazine] (Example 460) instead of 5-fluoro-4-(2-(5-fluoropyridin-2-yl)-5,6-dihydro-4H-pyrrolo[1,2-b]pyrazol-3-yl)-6-methyl-1H-pyrazolo[3,4-b]pyridine (Example 9). MS (ESI): mass calcd. for C₁₉H₁₄ClFN₆O 396.1; m/z found 397.1 [M+H]⁺. ¹H NMR (400 MHz, CD₃OD): δ 8.46 (d, J=4.9 Hz, 1H), 8.22-8.10 (m, 1H), 7.77-7.68 (m, 1H), 7.64-7.47 (m, 1H), 7.08 (d, J=4.9 Hz, 1H), 4.29-4.17 (m, 2H), 4.13 (d, J=11.7 Hz, 2H), 0.90 (s, 4H).

### Example 459: 3′-(5-Fluoro-1H-pyrazolo[3,4-b]pyridin-4-yl)-2′-(5-fluoropyridin-2-yl)-5′H,7′H-spiro[cyclopropane-1,6′-pyrazolo[5,1-b][1,3]oxazine]

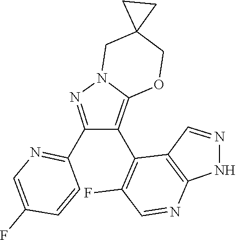 [SMILES: Fc1ccc(-c2nn3c(c2-c2c(F)cnc4[nH]ncc24)OCC2(CC2)C3)nc1]

The title compound was prepared in a manner analogous to Example 1, Steps A-B, except using lithium 2-(2′-(5-fluoropyridin-2-yl)-5′H,7′H-spiro[cyclopropane-1,6′-pyrazolo[5,1-b][1,3]oxazin]-3′-yl)-2-hydroxy-4,4,5,5-tetramethyl-1,3,2-dioxaborolan-2-uide (Intermediate 232) instead of 3-bromo-2-(5-fluoropyridin-2-yl)-6,7-dihydro-4H-pyrazolo[5,1-c][1,4]oxazine (Intermediate 37); and 2-[(5-fluoro-4-iodo-pyrazolo[3,4-b]pyridin-1-yl)methoxy]ethyl-trimethylsilane (Intermediate 204) instead of 4-(4,4,5,5-tetramethyl-1,3,2-dioxaborolan-2-yl)-1-((2-(trimethylsilyl)ethoxy)methyl)-1H-pyrrolo[2,3-b]pyridine (Intermediate 21) in Step A. MS (ESI): mass calcd. for C₁₉H₁₄F₂N₆O, 380.1; m/z found, 381.1 [M+H]⁺. ¹H NMR (600 MHz, DMSO-d₆) δ 8.44 (d, J=2.8 Hz, 1H), 8.30 (d, J=3.0 Hz, 1H), 7.91 (m, 1H), 7.85 (s, 1H), 7.78 (m, 1H), 4.24 (s, 2H), 4.19 (s, 2H), 0.86 (d, J=22.1 Hz, 4H). N—H proton not observed.

### Example 460: 2′-(5-Fluoropyridin-2-yl)-3′-(1H-pyrazolo[3,4-b]pyridin-4-yl)-5′H,7′H-spiro[cyclopropane-1,6′-pyrazolo[5,1-b][1,3]oxazine]

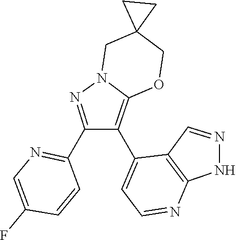 [SMILES: Fc1ccc(-c2nn3c(c2-c2ccnc4[nH]ncc24)OCC2(CC2)C3)nc1]

The title compound was prepared in a manner analogous to Example 1, Steps A-B, except using 3′-bromo-2′-(5-fluoropyridin-2-yl)-5′H,7′H-spiro[cyclopropane-1,6′-pyrazolo[5,1-b][1,3]oxazine](Intermediate 196) instead of 3-bromo-2-(5-fluoropyridin-2-yl)-6,7-dihydro-4H-pyrazolo[5,1-c][1,4]oxazine (Intermediate 37); and 4-(4,4,5,5-tetramethyl-1,3,2-dioxaborolan-2-yl)-1-((2-(trimethylsilyl)ethoxy)methyl)-1H-pyrazolo[3,4-b]pyridine (Intermediate 23) instead of 4-(4,4,5,5-tetramethyl-1,3,2-dioxaborolan-2-yl)-1-((2-(trimethylsilyl)ethoxy)methyl)-1H-pyrrolo[2,3-b]pyridine (Intermediate 21) in Step A. MS (ESI): mass calcd. for C₁₉H₁₅FN₆O, 362.1; m/z found, 363.1 [M+H]⁺. ¹H NMR (600 MHz, DMSO-d₆): δ 13.42 (s, 1H), 8.38 (d, J=4.9 Hz, 1H), 8.33 (d, J=2.9 Hz, 1H), 7.86-7.77 (m, 2H), 7.47 (d, J=1.4 Hz, 1H), 6.97 (d, J=4.8 Hz, 1H), 4.27 (s, 2H), 4.18 (s, 2H), 0.87 (dt, J=5.7, 1.7 Hz, 4H).

### Example 461: 2′-(4-Fluorophenyl)-3′-(6-methyl-1H-pyrazolo[3,4-b]pyridin-4-yl)-5′H,7′H-spiro[cyclopropane-1,6′-pyrazolo[5,1-b][1,3]oxazine]

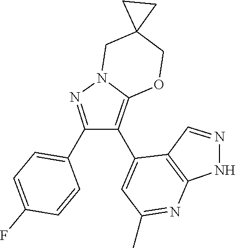 [SMILES: Cc1cc(-c2c(-c3ccc(F)cc3)nn3c2OCC2(CC2)C3)c2cn[nH]c2n1]

The title compound was prepared in a manner analogous to Example 1, Steps A-B, except using 3′-bromo-2′-(4-fluorophenyl)-5′H,7′H-spiro[cyclopropane-1,6′-pyrazolo[5,1-b][1,3]oxazine](Intermediate 195) instead of 3-bromo-2-(5-fluoropyridin-2-yl)-6,7-dihydro-4H-pyrazolo[5,1-c][1,4]oxazine (Intermediate 37); and 1-(4-methoxybenzyl)-6-methyl-4-(4,4,5,5-tetramethyl-1,3,2-dioxaborolan-2-yl)-1H-pyrazolo[3,4-b]pyridine (Intermediate 217) instead of 4-(4,4,5,5-tetramethyl-1,3,2-dioxaborolan-2-yl)-1-((2-(trimethylsilyl)ethoxy)methyl)-1H-pyrrolo[2,3-b]pyridine (Intermediate 21) in Step A. MS (ESI): mass calcd. for C₂₁H₁₈FN₅O 375.2; m/z found, 376.1 [M+H]⁺. ¹H NMR (500 MHz, CD₃OD): δ 7.42 (s, 1H), 7.41-7.32 (m, 2H), 7.11-6.97 (m, 2H), 6.94 (s, 1H), 4.23 (s, 2H), 4.13 (s, 2H), 2.54 (s, 3H), 0.92 (dt, J=7.5, 2.0 Hz, 4H). N—H proton not observed.

### Example 462: 2′-(5-Fluoropyridin-2-yl)-3′-(6-methyl-1H-pyrazolo[3,4-b]pyridin-4-yl)-5′H,7′H-spiro[cyclopropane-1,6′-pyrazolo[5,1-b][1,3]oxazine]

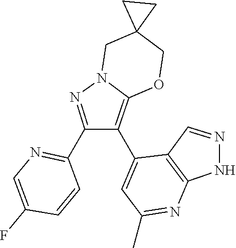 [SMILES: Cc1cc(-c2c(-c3ccc(F)cn3)nn3c2OCC2(CC2)C3)c2cn[nH]c2n1]

The title compound was prepared in a manner analogous to Example 1, Steps A-B, except using 3′-bromo-2′-(5-fluoropyridin-2-yl)-5′H,7′H-spiro[cyclopropane-1,6′-pyrazolo[5,1-b][1,3]oxazine](Intermediate 196) instead of 3-bromo-2-(5-fluoropyridin-2-yl)-6,7-dihydro-4H-pyrazolo[5,1-c][1,4]oxazine (Intermediate 37); and 1-(4-methoxybenzyl)-6-methyl-4-(4,4,5,5-tetramethyl-1,3,2-dioxaborolan-2-yl)-1H-pyrazolo[3,4-b]pyridine (Intermediate 217) instead of 4-(4,4,5,5-tetramethyl-1,3,2-dioxaborolan-2-yl)-1-((2-(trimethylsilyl)ethoxy)methyl)-1H-pyrrolo[2,3-b]pyridine (Intermediate 21) in Step A; the reaction mixture was heated at 60° C. for 6 hours instead of room temperature in Step B. MS (ESI): mass calcd. for C₂₀H₁₇FN₆O 376.1; m/z found 377.1 [M+H]⁺. ¹H NMR (600 MHz, DMSO-d₆) δ 13.19 (s, 1H), 8.32 (d, J=3.0 Hz, 1H), 7.87-7.76 (m, 2H), 7.28 (d, J=1.5 Hz, 1H), 6.89 (s, 1H), 4.26 (s, 2H), 4.18 (s, 2H), 3.31 (s, 3H), 0.87 (d, J=5.0 Hz, 4H).

### Example 463: 3′-(5-Fluoro-6-methyl-1H-pyrazolo[3,4-b]pyridin-4-yl)-2′-(5-fluoropyridin-2-yl)-5′H,7′H-spiro[cyclopropane-1,6′-pyrazolo[5,1-b][1,3]oxazine]

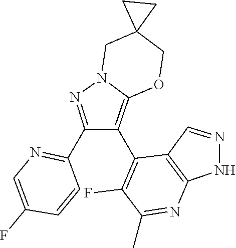 [SMILES: Cc1nc2[nH]ncc2c(-c2c(-c3ccc(F)cn3)nn3c2OCC2(CC2)C3)c1F]

The title compound was prepared in a manner analogous to Example 1, Steps A-B, except using 2′-(5-fluoropyridin-2-yl)-3′-(4,4,5,5-tetramethyl-1,3,2-dioxaborolan-2-yl)-5′H,7′H-spiro[cyclopropane-1,6′-pyrazolo[5,1-b][1,3]oxazine] (Intermediate 233) instead of 3-bromo-2-(5-fluoropyridin-2-yl)-6,7-dihydro-4H-pyrazolo[5,1-c][1,4]oxazine (Intermediate 37); and 5-fluoro-4-iodo-6-methyl-1-((2-(trimethylsilyl)ethoxy)methyl)-1H-pyrazolo[3,4-b]pyridine (Intermediate 242) instead of 4-(4,4,5,5-tetramethyl-1,3,2-dioxaborolan-2-yl)-1-((2-(trimethylsilyl)ethoxy)methyl)-1H-pyrrolo[2,3-b]pyridine (Intermediate 21) in Step A. MS (ESI): mass calcd. for C₂₀H₁₆F₂N₆O, 394.1; m/z found, 395.1 [M+H]⁺. ¹H NMR (600 MHz, DMSO-d₆): δ 13.41 (s, 1H), 8.27 (d, J=3.0 Hz, 1H), 7.90 (dd, J=8.8, 4.5 Hz, 1H), 7.77 (td, J=8.8, 3.0 Hz, 1H), 7.67 (d, J=1.4 Hz, 1H), 4.24 (s, 2H), 4.19 (s, 2H), 3.31 (s, 3H), 0.91-0.82 (m, 4H).

### Example 464: 3′-(3-Chloro-6-methyl-1H-pyrazolo[3,4-b]pyridin-4-yl)-2′-(5-fluoropyridin-2-yl)-5′H,7′H-spiro[cyclopropane-1,6′-pyrazolo[5,1-b][1,3]oxazine]

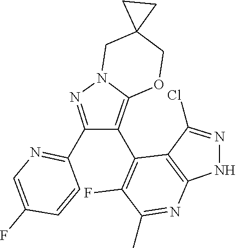 [SMILES: Cc1nc2[nH]nc(Cl)c2c(-c2c(-c3ccc(F)cn3)nn3c2OCC2(CC2)C3)c1F]

The title compound was prepared in a manner analogous to Example 224, except using 2′-(5-fluoropyridin-2-yl)-3′-(6-methyl-1H-pyrazolo[3,4-b]pyridin-4-yl)-5′H,7′H-spiro[cyclopropane-1,6′-pyrazolo[5,1-b][1,3]oxazine] (Example 462) instead of Example 9. MS (ESI): mass calcd. for C₂₀H₁₆ClFN₆O 410.1; m/z found 411.1 [M+H]⁺. ¹H NMR (400 MHz, CD₃OD): δ 8.15 (d, J=2.9 Hz, 1H), 7.72 (dd, J=8.9, 4.5 Hz, 1H), 7.59-7.49 (m, 1H), 7.01 (s, 1H), 4.30-4.18 (m, 2H), 4.13 (dd, J=11.8, 4.1 Hz, 2H), 2.62 (s, 3H), 0.90 (s, 4H). N—H proton not observed.

### Example 465: 2′-(4-Fluorophenyl)-3′-(6-methyl-1H-pyrazolo[3,4-d]pyrimidin-4-yl)-5′H,7′H-spiro[cyclopropane-1,6′-pyrazolo[5,1-b][1,3]oxazine]

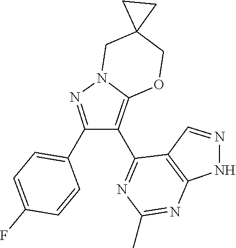 [SMILES: Cc1nc(-c2c(-c3ccc(F)cc3)nn3c2OCC2(CC2)C3)c2cn[nH]c2n1]

Step A. 2′-(4-Fluorophenyl)-3′-(6-methyl-1-((2-(trimethylsilyl)ethoxy)methyl)-1H-pyrazolo[3,4-d]pyrimidin-4-yl)-5′H,7′H-spiro[cyclopropane-1,6′-pyrazolo[5,1-b][1,3]oxazine]. A solution of lithium 2-(2′-(4-fluorophenyl)-5′H,7′H-spiro[cyclopropane-1,6′-pyrazolo[5,1-b][1,3]oxazin]-3′-yl)-2-hydroxy-4,4,5,5-tetramethyl-1,3,2-dioxaborolan-2-uide (Intermediate 231, 61 mg, 0.155 mmol), 4-chloro-6-methyl-1-((2-(trimethylsilyl)ethoxy)methyl)-1H-pyrazolo[3,4-d]pyrimidine (Intermediate 185, 69.4 mg, 0.232 mmol), [1,1′-bis(di-tert-butylphosphino)ferrocene]dichloropalladium(II) (10.15 mg, 0.0155 mmol), and potassium phosphate (101 mg, 0.464 mmol), in dry 1,4 dioxane (1.5 mL) was flushed with nitrogen, and heated employing microwave irradiation at 120° C. for 1 hour. The reaction mixture was filtered through a SiliaPrep SPE Thiol cartridge and washed with DCM, EtOAc, and MeOH. The combined organics were concentrated under reduced pressure. Purification (reverse phase using ACN/20 mM NH₄OH 0-100%) afforded the title compound (25 mg, 32%).

Step B. 2′-(4-Fluorophenyl)-3′-(6-methyl-1H-pyrazolo[3,4-d]pyrimidin-4-yl)-5′H,7′H-spiro[cyclopropane-1,6′-pyrazolo[5,1-b][1,3]oxazine]. The title compound was prepared in a manner analogous to Example 1, Step B, except using 2′-(4-fluorophenyl)-3′-(6-methyl-1-((2-(trimethylsilyl)ethoxy)methyl)-1H-pyrazolo[3,4-d]pyrimidin-4-yl)-5′H,7′H-spiro[cyclopropane-1,6′-pyrazolo[5,1-b][1,3]oxazine] instead of 2-(5-fluoropyridin-2-yl)-3-(1-((2-(trimethylsilyl)ethoxy)methyl)-1H-pyrrolo[2,3-b]pyridin-4-yl)-6,7-dihydro-4H-pyrazolo[5,1-c][1,4]oxazine (Example 1, Step A). MS (ESI): mass calcd. for C₂₀H₁₇FN₆O, 376.1; m/z found, 377.2 [M+H]⁺. ¹H NMR (600 MHz, DMSO-d₆) δ 7.84 (s, 1H), 7.51 (dd, J=8.9, 5.6 Hz, 2H), 7.13 (m, 2H), 4.23 (s, 2H), 4.11 (s, 2H), 2.42 (s, 3H), 0.84 (s, 4H). N—H proton not observed.

### Example 466: 2′-(4-Fluorophenyl)-3′-(pyrazolo[1,5-a]pyridin-5-yl)-5′H,7′H-spiro[cyclopropane-1,6′-pyrazolo[5,1-b][1,3]oxazine]

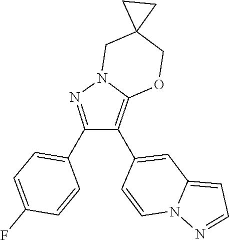 [SMILES: Fc1ccc(-c2nn3c(c2-c2ccn4nccc4c2)OCC2(CC2)C3)cc1]

The title compound was prepared in a manner analogous to Example 1, Step A, except using 3′-bromo-2′-(4-fluorophenyl)-5′H,7′H-spiro[cyclopropane-1,6′-pyrazolo[5,1-b][1,3]oxazine](Intermediate 195) instead of 3-bromo-2-(5-fluoropyridin-2-yl)-6,7-dihydro-4H-pyrazolo[5,1-c][1,4]oxazine (Intermediate 37); and pyrazolo[1,5-a]pyridine-5-boronic acid pinacol ester instead of 4-(4,4,5,5-tetramethyl-1,3,2-dioxaborolan-2-yl)-1-((2-(trimethylsilyl)ethoxy)methyl)-1H-pyrrolo[2,3-b]pyridine (Intermediate 21). MS (ESI): mass calcd. for C₂₁H₁₇FN₄O 360.1; m/z found 361.1 [M+H]⁺. ¹H NMR (500 MHz, CD₃OD): δ 8.35 (dt, J=7.3, 1.0 Hz, 1H), 7.87 (d, J=2.4 Hz, 1H), 7.61-7.37 (m, 3H), 7.22-7.00 (m, 2H), 6.68 (dd, J=7.3, 1.9 Hz, 1H), 6.45 (dd, J=2.4, 0.9 Hz, 1H), 4.22 (s, 2H), 4.09 (s, 2H), 1.00-0.73 (m, 4H).

### Example 467: 6,6-Difluoro-2-(4-fluorophenyl)-3-(1H-pyrazolo[3,4-b]pyridin-4-yl)-5,7-dihydropyrazolo[5,1-b][1,3]oxazine

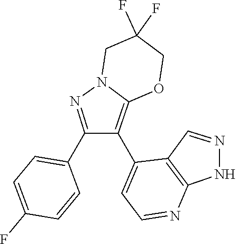 [SMILES: Fc1ccc(-c2nn3c(c2-c2ccnc4[nH]ncc24)OCC(F)(F)C3)cc1]

The title compound was prepared in a manner analogous to Example 1, Steps A-B, except using 3-bromo-6,6-difluoro-2-(4-fluorophenyl)-6,7-dihydro-5H-pyrazolo[5,1-b][1,3]oxazine (Intermediate 193) instead of 3-bromo-2-(5-fluoropyridin-2-yl)-6,7-dihydro-4H-pyrazolo[5,1-c][1,4]oxazine (Intermediate 37); and 4-(4,4,5,5-tetramethyl-1,3,2-dioxaborolan-2-yl)-1-((2-(trimethylsilyl)ethoxy)methyl)-1H-pyrazolo[3,4-b]pyridine (Intermediate 23) instead of 4-(4,4,5,5-tetramethyl-1,3,2-dioxaborolan-2-yl)-1-((2-(trimethylsilyl)ethoxy)methyl)-1H-pyrrolo[2,3-b]pyridine (Intermediate 21) in Step A. MS (ESI): mass calcd. for C₁₈H₁₂F₃N₅O 371.1; m/z found 372.1 [M+H]⁺. ¹H NMR (400 MHz, DMSO-d₆): δ 13.55 (s, 1H), 8.39 (d, J=4.9 Hz, 1H), 7.51 (s, 1H), 7.35-7.28 (m, 2H), 7.16-7.08 (m, 2H), 6.89 (d, J=4.6 Hz, 1H), 4.92-4.65 (m, 4H).

### Example 468: 6,6-Difluoro-2-(4-fluorophenyl)-3-(1H-pyrazolo[4,3-b]pyridin-7-yl)-5,7-dihydropyrazolo[5,1-b][1,3]oxazine

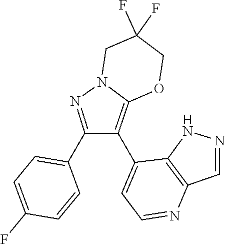 [SMILES: Fc1ccc(-c2nn3c(c2-c2ccnc4cn[nH]c24)OCC(F)(F)C3)cc1]

The title compound was prepared in a manner analogous to Example 279, except using 3-bromo-6,6-difluoro-2-(4-fluorophenyl)-6,7-dihydro-5H-pyrazolo[5,1-b][1,3]oxazine (Intermediate 193) instead of 3-bromo-6,6-difluoro-2-(4-fluorophenyl)-4,5,6,7-tetrahydropyrazolo[1,5-a]pyridine (Intermediate 52); and 7-bromo-1-((2-(trimethylsilyl)ethoxy)methyl)-1H-pyrazolo[4,3-b]pyridine (Intermediate 211) instead of 4-Chloro-6-methyl-1-((2-(trimethylsilyl)ethoxy)methyl)-1H-pyrazolo[3,4-d]pyrimidine (Intermediate 185). MS (ESI): mass calcd. for C₁₈H₁₂F₃N₅O, 371.1; m/z found, 372.4 [M+H]⁺. ¹H NMR (400 MHz, DMSO-d₆): δ 13.15 (br s, 1H), 8.40 (d, J=4.6 Hz, 1H), 8.29 (s, 1H), 7.37-7.29 (m, 2H), 7.10 (s, 2H), 6.98 (d, J=4.4 Hz, 1H), 4.85 (t, J=12.8 Hz, 2H), 4.73 (t, J=11.0 Hz, 2H).

### Example 469: 6,6-Difluoro-2-(4-fluorophenyl)-3-(6-methyl-1H-pyrazolo[3,4-d]pyrimidin-4-yl)-5,7-dihydropyrazolo[5,1-b][1,3]oxazine

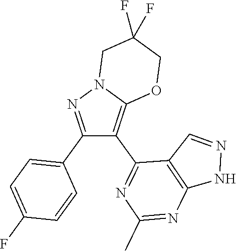 [SMILES: Cc1nc(-c2c(-c3ccc(F)cc3)nn3c2OCC(F)(F)C3)c2cn[nH]c2n1]

The title compound was prepared in a manner analogous to Example 279, except using 3-bromo-6,6-difluoro-2-(4-fluorophenyl)-6,7-dihydro-5H-pyrazolo[5,1-b][1,3]oxazine (Intermediate 193) instead of 3-bromo-6,6-difluoro-2-(4-fluorophenyl)-4,5,6,7-tetrahydropyrazolo[1,5-a]pyridine (Intermediate 52). MS (ESI): mass calcd. for C₁₈H₁₃F₃N₆O 386.1; m/z found 387.0 [M+H]⁺. ¹H NMR (400 MHz, DMSO-d₆): δ 7.82 (s, 1H), 7.54-7.45 (m, 2H), 7.17-7.08 (m, 2H), 4.90-4.75 (m, 4H), 2.51 (s, 3H). N—H proton not observed.

### Example 470: 3-(3-Chloro-1H-pyrazolo[3,4-b]pyridin-4-yl)-6,6-difluoro-2-(4-fluorophenyl)-6,7-dihydro-5H-pyrazolo[5,1-b][1,3]oxazine

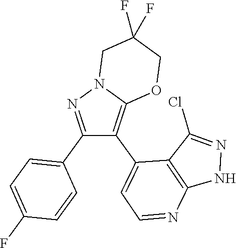 [SMILES: Fc1ccc(-c2nn3c(c2-c2ccnc4[nH]nc(Cl)c24)OCC(F)(F)C3)cc1]

The title compound was prepared in a manner analogous to Example 224, except using 6,6-difluoro-2-(4-fluorophenyl)-3-(1H-pyrazolo[3,4-b]pyridin-4-yl)-6,7-dihydro-5H-pyrazolo[5,1-b][1,3]oxazine (Example 467) instead of Example 9. MS (ESI): mass calcd. for C₁₈H₁₁ClF₃N₅O, 405.1; m/z found, 406.1 [M+H]⁺. ¹H NMR (400 MHz, CD₃OD): δ 8.47 (d, J=4.8 Hz, 1H), 7.41-7.27 (m, 2H), 7.05-6.90 (m, 3H), 4.81-4.68 (m, 2H), 4.59 (tt, J=11.2, 5.6 Hz, 2H).

### Example 471: 2,2-Difluoro-3′-(5-fluoro-1H-pyrazolo[3,4-b]pyridin-4-yl)-2′-(4-fluorophenyl)-5′H,7′H-spiro[cyclopropane-1,6′-pyrazolo[5,1-b][1,3]oxazine]

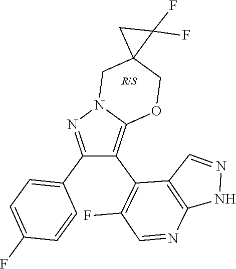 [SMILES: Fc1ccc(-c2nn3c(c2-c2c(F)cnc4[nH]ncc24)OCC2(C3)CC2(F)F)cc1]

Step A. 2,2-Difluoro-3′-(5-fluoro-1-((2-(trimethylsilyl)ethoxy)methyl)-1H-pyrazolo[3,4-b]pyridin-4-yl)-2′-(4-fluorophenyl)-5′H,7′H-spiro[cyclopropane-1,6′-pyrazolo[5,1-b][1,3]oxazine]. A solution of lithium 2-(2,2-difluoro-2′-(4-fluorophenyl)-5′H,7′H-spiro[cyclopropane-1,6′-pyrazolo[5,1-b][1,3]oxazin]-3′-yl)-2-hydroxy-4,4,5,5-tetramethyl-1,3,2-dioxaborolan-2-uide (Intermediate 234, 110 mg, 0.256 mmol), 5-fluoro-4-iodo-1-((2-(trimethylsilyl)ethoxy)methyl)-1H-pyrazolo[3,4-b]pyridine (Intermediate 231, 118.5 mg, 0.294 mmol), CataCXium Pd G4 (28 mg, 0.0384 mmol), and Cs₂CO₃ (250 mg, 0.767 mmol), in dry 1,4 dioxane (2.4 mL) was flushed with nitrogen, and heated to 120° C. employing microwave irradiation for 1 hour. The reaction, mixture was filtered through a SiliaPrep SPE Thiol cartridge and washed with DCM, EtOAc and MeOH. The combined organics were concentrated under reduced pressure. Purification of the resulting residue (reverse phase chromatography using ACN/20 mM NH₄OH 0-100%) afforded the title compound as a white solid.

Step B. 2,2-Difluoro-3′-(5-fluoro-1H-pyrazolo[3,4-b]pyridin-4-yl)-2′-(4-fluorophenyl)-5′H,7′H-spiro[cyclopropane-1,6′-pyrazolo[5,1-b][1,3]oxazine]. The title compound was prepared in a manner analogous to Example 1, Step B, except using 2,2-difluoro-3′-(5-fluoro-1-((2-(trimethylsilyl)ethoxy)methyl)-1H-pyrazolo[3,4-b]pyridin-4-yl)-2′-(4-fluorophenyl)-5′H,7′H-spiro[cyclopropane-1,6′-pyrazolo[5,1-b][1,3]oxazine] instead of 2-(5-fluoropyridin-2-yl)-3-(1-((2-(trimethylsilyl)ethoxy)methyl)-1H-pyrrolo[2,3-b]pyridin-4-yl)-6,7-dihydro-4H-pyrazolo[5,1-c][1,4]oxazine (Example 1, Step A). MS (ESI): mass calcd. for C₂₀H₁₃F₄N₅O 415.1; m/z found 416.1 [M+H]⁺. ¹H NMR (600 MHz, DMSO-d₆) δ 13.73 (s, 1H), 8.51 (d, J=2.6 Hz, 1H), 7.72 (s, 1H), 7.40-7.32 (m, 2H), 7.17-7.09 (m, 2H), 4.62 (d, J=11.5 Hz, 1H), 4.56-4.38 (m, 2H), 4.33 (d, J=12.6 Hz, 1H), 2.09-1.98 (m, 2H).

### Example 472: (*S)-2,2-Difluoro-2′-(4-fluorophenyl)-3′-(3-methyl-1H-pyrazolo[3,4-b]pyridin-4-yl)-5′H,7′H-spiro[cyclopropane-1,6′-pyrazolo[5,1-b][1,3]oxazine]

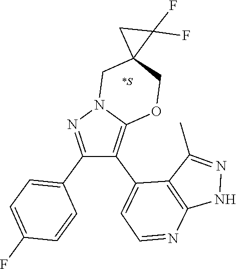 [SMILES: Cc1n[nH]c2nccc(-c3c(-c4ccc(F)cc4)nn4c3OC[C@@]3(C4)CC3(F)F)c12]

Step A: 2,2-Difluoro-2′-(4-fluorophenyl)-3′-(3-methyl-1-((2-(trimethylsilyl)ethoxy)methyl)-1H-pyrazolo[3,4-b]pyridin-4-yl)-5′H,7′H-spiro[cyclopropane-1,6′-pyrazolo[5,1-b][1,3]oxazine].

A solution of lithium 2-(2,2-difluoro-2′-(4-fluorophenyl)-5′H,7′H-spiro[cyclopropane-1,6′-pyrazolo[5,1-b][1,3]oxazin]-3′-yl)-2-hydroxy-4,4,5,5-tetramethyl-1,3,2-dioxaborolan-2-uide (Intermediate 234, 190 mg, 0.442 mmol), 4-iodo-3-methyl-1-((2-(trimethylsilyl)ethoxy)methyl)-1H-pyrazolo[3,4-b]pyridine (Intermediate 202, 204.6 mg, 0.526 mmol), bis(1-adamantyl)-butylphosphane;methanesulfonic acid;n-methyl-2-phenylaniline;palladium (48 mg, 0.0663 mmol), and Cs₂CO₃ (431 mg, 1.325 mmol), in THF (4 mL), and DI water (0.155 mL, 8.6 mmol), flushed with nitrogen, was reacted in a sealed tube, at 90° C. for 20 hours. The reaction mixture was cooled, and diluted with EtOAc, DCM, and MeOH and filtered through a SiliaPrep SPE Thiol cartridge, concentrated, and purified by reverse phase chromatography using ACN/20 mM NH₄OH 0-100% to deliver a white solid (56 mg, 23%).

Step B: (*S)-2,2-Difluoro-2′-(4-fluorophenyl)-3′-(3-methyl-1H-pyrazolo[3,4-b]pyridin-4-yl)-5′H,7′H-spiro[cyclopropane-1,6′-pyrazolo[5,1-b][1,3]oxazine]. The title compound was prepared in a manner analogous to Example 1, Step B. Chiral SFC purification (Stationary phase: Chiralpak IB N₃ Sum 250×21 mm, Mobile phase: 25% methanol with 0.2% triethylamine, 75% CO₂) afforded the title compound: MS (ESI): mass calcd. for C₂₁H₁₆F₃N₅O 415.1; m/z found 416.1 [M+H]⁺. ¹H NMR (600 MHz, DMSO-d₆): δ 13.26 (d, J=6.7 Hz, 1H), 8.41 (dd, J=7.1, 4.7 Hz, 1H), 7.34 (dd, J=8.3, 5.6 Hz, 2H), 7.10 (td, J=8.9, 3.6 Hz, 2H), 6.84 (dd, J=32.3, 4.6 Hz, 1H), 4.61-4.25 (m, 4H), 2.17 (d, J=11.3 Hz, 3H), 1.26 (s, 2H); and (*R)-2,2-Difluoro-2′-(4-fluorophenyl)-3′-(3-methyl-1H-pyrazolo[3,4-b]pyridin-4-yl)-5′H,7′H-spiro[cyclopropane-1,6′-pyrazolo[5,1-b][1,3]oxazine] (Example 473).

### Example 473: (*R)-2,2-Difluoro-2′-(4-fluorophenyl)-3′-(3-methyl-1H-pyrazolo[3,4-b]pyridin-4-yl)-5′H,7′H-spiro[cyclopropane-1,6′-pyrazolo[5,1-b][1,3]oxazine]

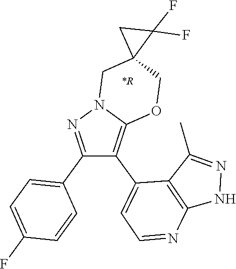 [SMILES: Cc1n[nH]c2nccc(-c3c(-c4ccc(F)cc4)nn4c3OC[C@]3(C4)CC3(F)F)c12]

The title compound was isolated from chiral SFC purification of Example 472. MS (ESI): mass calcd. for C₂₁H₁₆F₃N₅O 411.1; m/z found 412.1 [M+H]⁺. ¹H NMR (600 MHz, DMSO-d₆): δ 13.26 (d, J=6.7 Hz, 1H), 8.42-8.39 (m, 1H), 7.36-7.32 (m, 2H), 7.13-7.07 (m, 2H), 6.88-6.80 (m, 1H), 4.62-4.24 (m, 4H), 2.17 (d, J=11.3 Hz, 3H), 1.26 (s, 2H).

### Example 474: *S-2,2-Difluoro-2′-(4-fluorophenyl)-3′-6-methyl-1H-pyrazolo[3,4-b]pyridin-4-yl)-5′H,7′H-spiro[cyclopropane-1,6′-pyrazolo[5,1-b][1,3]oxazine]

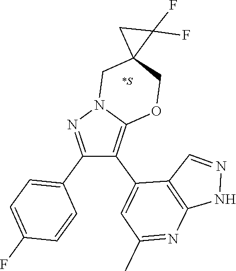 [SMILES: Cc1cc(-c2c(-c3ccc(F)cc3)nn3c2OC[C@@]2(C3)CC2(F)F)c2cn[nH]c2n1]

The title compound was prepared in a manner analogous to Example 1, Steps A-B, except using 3′-bromo-2,2-difluoro-2′-(4-fluorophenyl)-5′H,7′H-spiro[cyclopropane-1,6′-pyrazolo[5,1-b][1,3]oxazine] (Intermediate 197), instead of 3-bromo-2-(5-fluoropyridin-2-yl)-6,7-dihydro-4H-pyrazolo[5,1-c][1,4]oxazine (Intermediate 37); and 1-(4-methoxybenzyl)-6-methyl-4-(4,4,5,5-tetramethyl-1,3,2-dioxaborolan-2-yl)-1H-pyrazolo[3,4-b]pyridine (Intermediate 217) instead of 4-(4,4,5,5-tetramethyl-1,3,2-dioxaborolan-2-yl)-1-((2-(trimethylsilyl)ethoxy)methyl)-1H-pyrrolo[2,3-b]pyridine (Intermediate 21) in Step A. Purification (chiral SFC; Stationary phase: Chiralcel OJ-H Sum 250×21 mm, Mobile phase: 30% methanol with 0.2% triethylamine, 70% CO₂), flow rate 42 mL/min, monitor at 220 nm) afforded the title compound: MS (ESI): mass calcd. for C₂₁H₁₆F₃N₅O 411.1; m/z found 412.1 [M+H]⁺; ¹H NMR (600 MHz, CD₃OD): δ 7.46 (s, 1H), 7.45-7.40 (m, 2H), 7.11-7.04 (m, 2H), 6.98 (s, 1H), 4.63 (m, 1H), 4.47 (m, 2H), 4.38 (d, J=12.4 Hz, 1H), 2.59 (s, 3H), 1.34 (d, J=15.2 Hz, 2H); and (*R)-2,2-Difluoro-2′-(4-fluorophenyl)-3′-(6-methyl-1H-pyrazolo[3,4-b]pyridin-4-yl)-5′H,7′H-spiro[cyclopropane-1,6′-pyrazolo[5,1-b][1,3]oxazine] (Example 475).

### Example 475: (*R)-2,2-Difluoro-2′-(4-fluorophenyl)-3′-(6-methyl-1H-pyrazolo[3,4-b]pyridin-4-yl)-5′H,7′H-spiro[cyclopropane-1,6′-pyrazolo[5,1-b][1,3]oxazine]

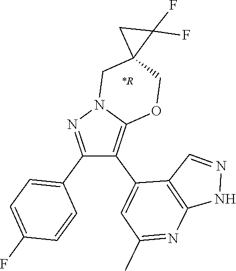 [SMILES: Cc1cc(-c2c(-c3ccc(F)cc3)nn3c2OC[C@]2(C3)CC2(F)F)c2cn[nH]c2n1]

The title compound was isolated from chiral SFC purification of Example 474. MS (ESI): mass calcd. for C₂₁H₁₆F₃N₅O 411.1; m/z found 412.1 [M+H]⁺. ¹H NMR (600 MHz, CD₃OD) δ 7.46 (s, 1H), 7.45-7.40 (m, 2H), 7.11-7.04 (m, 2H), 6.98 (s, 1H), 4.63 (m, 1H), 4.47 (m, 2H), 4.38 (d, J=12.4 Hz, 1H), 2.59 (s, 3H), 1.34 (d, J=15.2 Hz, 2H). N—H proton is not observed.

### Example 476: (*S)-2,2-Difluoro-2′-(4-fluorophenyl)-3′-1H-pyrazolo[3,4-b]pyridin-4-yl)-5′H,7′H-spiro[cyclopropane-1,6′-pyrazolo[5,1-b][1,3]oxazine]

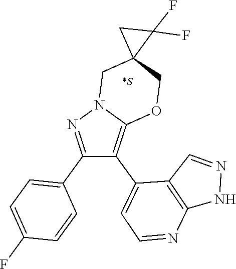 [SMILES: Fc1ccc(-c2nn3c(c2-c2ccnc4[nH]ncc24)OC[C@@]2(C3)CC2(F)F)cc1]

The title compound was prepared in a manner analogous to Example 1, Steps A-B, except using 3′-bromo-2,2-difluoro-2′-(4-fluorophenyl)-5′H,7′H-spiro[cyclopropane-1,6′-pyrazolo[5,1-b][1,3]oxazine] (Intermediate 197), instead of 3-bromo-2-(5-fluoropyridin-2-yl)-6,7-dihydro-4H-pyrazolo[5,1-c][1,4]oxazine (Intermediate 37 and 4-(4,4,5,5-tetramethyl-1,3,2-dioxaborolan-2-yl)-1-((2-(trimethylsilyl)ethoxy)methyl)-1H-pyrazolo[3,4-b]pyridine (Intermediate 23) instead of 4-(4,4,5,5-tetramethyl-1,3,2-dioxaborolan-2-yl)-1-((2-(trimethylsilyl)ethoxy)methyl)-1H-pyrrolo[2,3-b]pyridine (Intermediate 21) in Step A. Purification (chiral SFC; Stationary phase: Chiralcel OJ-H Sum 250×21 mm, Mobile phase: 35% methanol with 0.2% triethylamine, 65% CO₂), flow rate 42 mL/min, monitor at 220 nm) afforded the title compound: MS (ESI): mass calcd. for C₂₀H₁₄F₃N₅O 397.1; m/z found 398.1 [M+H]⁺; ¹H NMR (600 MHz, DMSO-d₆) δ 13.76 (s, 1H), 8.65 (d, J=4.8 Hz, 1H), 7.79 (d, J=1.4 Hz, 1H), 7.64-7.55 (m, 2H), 7.45-7.35 (m, 2H), 7.15 (d, J=4.8 Hz, 1H), 4.91-4.80 (m, 1H), 4.75-4.65 (m, 2H), 4.56 (d, J=12.4 Hz, 1H), 1.51 (d, J=26.2 Hz, 2H); and (*R)-2,2-Difluoro-2′-(4-fluorophenyl)-3′-(1H-pyrazolo[3,4-b]pyridin-4-yl)-5′H,7′H-spiro[cyclopropane-1,6′-pyrazolo[5,1-b][1,3]oxazine] (Example 477).

### Example 477: (*R)-2,2-Difluoro-2′-(4-fluorophenyl)-3′-1H-pyrazolo[3,4-b]pyridin-4-yl)-5′H,7′H-spiro[cyclopropane-1,6′-pyrazolo[5,1-b][1,3]oxazine]

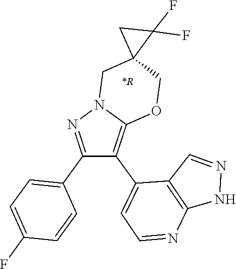 [SMILES: Fc1ccc(-c2nn3c(c2-c2ccnc4[nH]ncc24)OC[C@]2(C3)CC2(F)F)cc1]

The title compound was isolated from chiral SFC purification of Example 476. MS (ESI): mass calcd. for C₂₀H₁₄F₃N₅O 397.1; m/z found 398.1 [M+H]⁺. ¹H NMR (600 MHz, DMSO-d₆): δ 13.76 (s, 1H), 8.65 (d, J=4.8 Hz, 1H), 7.79 (d, J=1.4 Hz, 1H), 7.64-7.55 (m, 2H), 7.45-7.35 (m, 2H), 7.15 (d, J=4.8 Hz, 1H), 4.91-4.80 (m, 1H), 4.75-4.65 (m, 2H), 4.56 (d, J=12.4 Hz, 1H), 1.51 (d, J=26.2 Hz, 2H).

### Example 478: (*S)-2,2-Difluoro-2′-(5-fluoropyridin-2-yl)-3′-1H-pyrazolo[3,4-b]pyridin-4-yl)-5′H,7′H-spiro[cyclopropane-1,6′-pyrazolo[5,1-b][1,3]oxazine]

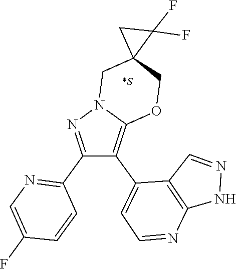 [SMILES: Fc1ccc(-c2nn3c(c2-c2ccnc4[nH]ncc24)OC[C@@]2(C3)CC2(F)F)nc1]

The title compound was prepared in a manner analogous to Example 1, Steps A-B, except using 2,2-difluoro-2′-(5-fluoropyridin-2-yl)-5′H,7′H-spiro[cyclopropane-1,6′-pyrazolo[5,1-b][1,3]oxazine] (Intermediate 209, instead of 3-bromo-2-(5-fluoropyridin-2-yl)-6,7-dihydro-4H-pyrazolo[5,1-c][1,4]oxazine (Intermediate 37); and 4-(4,4,5,5-tetramethyl-1,3,2-dioxaborolan-2-yl)-1-((2-(trimethylsilyl)ethoxy)methyl)-1H-pyrazolo[3,4-b]pyridine (Intermediate 23) instead of 4-(4,4,5,5-tetramethyl-1,3,2-dioxaborolan-2-yl)-1-((2-(trimethylsilyl)ethoxy)methyl)-1H-pyrrolo[2,3-b]pyridine (Intermediate 21) in Step A. Purification (chiral SFC: Stationary phase: Chiralpak IC 5 um 250×21 mm, Mobile phase: 25% methanol:isopropanol (1:1) with 0.2% isopropylamine, 75% CO₂), flow rate 42 mL/min, monitor at 220 nm) afforded the title compound: MS (ESI): mass calcd. for C₁₉H₁₃F₃N₆O 398.1; m/z found 399.1 [M+H]⁺; ¹H NMR (600 MHz, DMSO-d₆): δ 13.46 (s, 1H), 8.40 (d, J=4.8 Hz, 1H), 8.34 (d, J=2.9 Hz, 1H), 7.87-7.77 (m, 2H), 7.48 (d, J=1.3 Hz, 1H), 6.97 (d, J=4.8 Hz, 1H), 4.66 (d, J=11.4 Hz, 1H), 4.49 (m, 2H), 4.36 (d, J=12.7 Hz, 1H), 1.26 (s, 2H); and (*R)-2,2-Difluoro-2′-(5-fluoropyridin-2-yl)-3′-(1H-pyrazolo[3,4-b]pyridin-4-yl)-5′H,7′H-spiro[cyclopropane-1,6′-pyrazolo[5,1-b][1,3]oxazine] (Example 479).

### Example 479: (*R)-2,2-Difluoro-2′-(5-fluoropyridin-2-yl)-3′-1H-pyrazolo[3,4-b]pyridin-4-yl)-5′H,7′H-spiro[cyclopropane-1,6′-pyrazolo[5,1-b][1,3]oxazine]

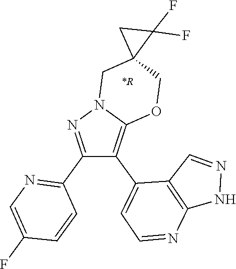 [SMILES: Fc1ccc(-c2nn3c(c2-c2ccnc4[nH]ncc24)OC[C@]2(C3)CC2(F)F)nc1]

The title compound was isolated from chiral SFC purification of Example 478. MS (ESI): mass calcd. for C₁₉H₁₃F₃N₆O 398.1; m/z found 399.1 [M+H]⁺. ¹H NMR (600 MHz, DMSO-d₆) δ 13.46 (s, 1H), 8.40 (d, J=4.8 Hz, 1H), 8.34 (d, J=2.9 Hz, 1H), 7.87-7.77 (m, 2H), 7.48 (d, J=1.3 Hz, 1H), 6.97 (d, J=4.8 Hz, 1H), 4.66 (d, J=11.4 Hz, 1H), 4.49 (m, 2H), 4.36 (d, J=12.7 Hz, 1H), 1.26 (s, 2H).

### Example 480: (5a*R,6a*S)-3-(5-Fluoro-1H-pyrazolo[3,4-b]pyridin-4-yl)-2-(5-fluoropyridin-2-yl)-5a,6,6a,7-tetrahydro-5H-cyclopropa[e]pyrazolo[5,1-b][1,3]oxazepine

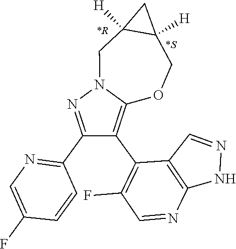 [SMILES: Fc1ccc(-c2nn3c(c2-c2c(F)cnc4[nH]ncc24)OC[C@@H]2C[C@@H]2C3)nc1]

The title compound was prepared in a manner analogous to (Example 236), except using cis-2-(5-fluoropyridin-2-yl)-3-iodo-5a,6,6a,7-tetrahydro-5H-cyclopropa[e]pyrazolo[5,1-b][1,3]oxazepine (Intermediate 201) instead of 3-bromo-2-(5-fluoropyridin-2-yl)-5,5-dimethyl-5,6-dihydro-4H-pyrrolo[1,2-b]pyrazole (Intermediate 152) and using iPrMgCl instead of n-BuLi. Purification (chiral SFC (WHELK-01 (250 mm×30 mm, 5 um) (isocratic elution: EtOH (containing 0.1% aq. NH₃): supercritical CO₂, 40%: 60% to 40%: 60% (v/v))) afforded the title compound: MS (ESI): mass calcd. for C₁₉H₁₄F₂N₆O 380.1; m/z found 381.1 [M+H]⁺; ¹H NMR (400 MHz, CDCl₃): δ 11.04 (br s, 1H), 8.40 (s, 1H), 8.33-8.21 (m, 1H), 7.81 (s, 1H), 7.58 (dd, J=4.5, 8.7 Hz, 1H), 7.39-7.30 (m, 1H), 4.98 (dd, J=7.2, 15.1 Hz, 1H), 4.73 (dd, J=5.5, 12.8 Hz, 1H), 4.37-4.17 (m, 2H), 2.05-1.83 (m, 1H), 1.8 0-1.73 (m, 1H), 1.19-1.09 (m, 1H), 0.84-0.74 (m, 1H); and (5a*S,6a*R)-3-(5-Fluoro-1H-pyrazolo[3,4-b]pyridin-4-yl)-2-(5-fluoropyridin-2-yl)-5a,6,6a,7-tetrahydro-5H-cyclopropa[e]pyrazolo[5,1-b][1,3]oxazepane (Example 481).

### Example 481: (5a*S,6a*R)-3-(5-Fluoro-1H-pyrazolo[3,4-b]pyridin-4-yl)-2-(5-fluoropyridin-2-yl)-5a,6,6a,7-tetrahydro-5H-cyclopropa[e]pyrazolo[5,1-b][1,3]oxazepine

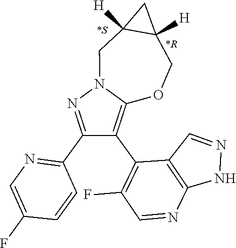 [SMILES: Fc1ccc(-c2nn3c(c2-c2c(F)cnc4[nH]ncc24)OC[C@H]2C[C@H]2C3)nc1]

The title compound was isolated from chiral SFC purification of Example 480. MS (ESI): mass calcd. for C₁₉H₁₄F₂N₆O 380.1; m/z found 381.0 [M+H]⁺. ¹H NMR (400 MHz, CDCl₃): δ 11.02 (br s, 1H), 8.40 (d, J=2.4 Hz, 1H), 8.28 (d, J=2.6 Hz, 1H), 7.81 (s, 1H), 7.58 (dd, J=4.3, 8.7 Hz, 1H), 7.38-7.29 (m, 1H), 4.99 (dd, J=7.2, 15.1 Hz, 1H), 4.73 (dd, J=5.5, 13.0 Hz, 1H), 4.36-4.17 (m, 2H), 1.99-1.89 (m, 1H), 1.81-1.70 (m, 1H), 1.17-1.09 (m, 1H), 0.79 (q, J=4.9 Hz, 1H).

### Example 482: (5a*R,6a*S)-3-(5-Fluoro-6-methyl-1H-pyrazolo[3,4-b]pyridin-4-yl)-2-(5-fluoropyridin-2-yl)-5a,6,6a,7-tetrahydro-5H-cyclopropa[e]pyrazolo[5,1-b][1,3]oxazepine

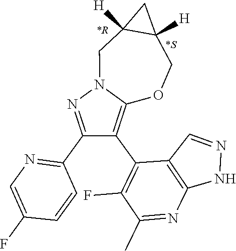 [SMILES: Cc1nc2[nH]ncc2c(-c2c(-c3ccc(F)cn3)nn3c2OC[C@H]2C[C@H]2C3)c1F]

The title compound was prepared in a manner analogous to Example 1, Steps A-B, except using cis-3-Bromo-2-(5-fluoropyridin-2-yl)-5a,6,6a,7-tetrahydro-5H-cyclopropa[e]pyrazolo[5,1-b][1,3]oxazepane (Intermediate 200) instead of 3-bromo-2-(5-fluoropyridin-2-yl)-6,7-dihydro-4H-pyrazolo[5,1-c][1,4]oxazine (Intermediate 37) and 5-Fluoro-6-methyl-4-(4,4,5,5-tetramethyl-1,3,2-dioxaborolan-2-yl)-1-((2-(trimethylsilyl)ethoxy)methyl)-1H-pyrazolo[3,4-b]pyridine (Intermediate 223) instead of 4-(4,4,5,5-tetramethyl-1,3,2-dioxaborolan-2-yl)-1-((2-(trimethylsilyl)ethoxy)methyl)-1H-pyrrolo[2,3-b]pyridine (Intermediate 21) in Step A. Purification (chiral SFC: DAICEL CHIRALPAK AD (250 mm×30 mm, 10 μm) (isocratic elution: EtOH (containing 0.1% aq. NH₃): supercritical CO₂, 35%: 65% to 35%: 65% (v/v)) afforded the title compound: MS (ESI): mass calcd. for C₂₀H₁₆F₂N₆O 394.1; m/z found 395.1 [M+H]⁺; ¹H NMR (400 MHz, DMSO-d₆) δ 13.39 (s, 1H), 8.22 (d, J=3.0 Hz, 1H), 7.86 (dd, J=4.5, 8.8 Hz, 1H), 7.78-7.70 (m, 1H), 7.63 (s, 1H), 4.77 (dd, J=7.2, 14.9 Hz, 1H), 4.69-4.61 (m, 1H), 4.58-4.45 (m, 2H), 2.47 (d, J=3.5 Hz, 3H), 1.93-1.81 (m, 1H), 1.73-1.60 (m, 1H), 1.02-0.95 (m, 1H), 0.88-0.80 (m, 1H); and (5a*S,6a*R)-3-(5-Fluoro-6-methyl-1H-pyrazolo[3,4-b]pyridin-4-yl)-2-(5-fluoropyridin-2-yl)-5a,6,6a,7-tetrahydro-5H-cyclopropa[e]pyrazolo[5,1-b][1,3]oxazepane (Example 483).

### Example 483: (5a*S,6a*R)-3-(5-Fluoro-6-methyl-1H-pyrazolo[3,4-b]pyridin-4-yl)-2-(5-fluoropyridin-2-yl)-5a,6,6a,7-tetrahydro-5H-cyclopropa[e]pyrazolo[5,1-b][1,3]oxazepine

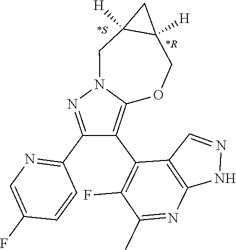 [SMILES: Cc1nc2[nH]ncc2c(-c2c(-c3ccc(F)cn3)nn3c2OC[C@@H]2C[C@@H]2C3)c1F]

The title compound was isolated from chiral SFC purification of Example 482. MS (ESI): mass calcd. for C₂₀H₁₆F₂N₆O 394.1; m/z found 395.1 [M+H]⁺. ¹H NMR (400 MHz, DMSO-d₆) δ 13.40 (br s, 1H), 8.22 (d, J=2.8 Hz, 1H), 7.86 (dd, J=4.5, 8.8 Hz, 1H), 7.78-7.70 (m, 1H), 7.64 (s, 1H), 4.77 (dd, J=7.2, 14.9 Hz, 1H), 4.65 (dd, J=5.5, 12.8 Hz, 1H), 4.58-4.44 (m, 2H), 2.47 (d, J=3.5 Hz, 3H), 1.93-1.81 (m, 1H), 1.73-1.59 (m, 1H), 1.03-0.94 (m, 1H), 0.89-0.80 (m, 1H).

### Example 484: 2-(5-Fluoropyridin-2-yl)-7-methyl-3-(1H-pyrazolo[3,4-b]pyridin-4-yl)-7,8-dihydro-4H,6H-pyrazolo[5,1-c][1,4]oxazepin-7-ol

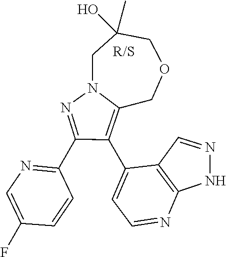 [SMILES: CC1(O)COCc2c(-c3ccnc4[nH]ncc34)c(-c3ccc(F)cn3)nn2C1]

The title compound was prepared in a manner analogous to Example 1, Steps A-B, except using 3-bromo-2-(5-fluoropyridin-2-yl)-7-methyl-7,8-dihydro-4H,6H-pyrazolo[5,1-c][1,4]oxazepin-7-ol (Intermediate 199) instead of 3-bromo-2-(5-fluoropyridin-2-yl)-6,7-dihydro-4H-pyrazolo[5,1-c][1,4]oxazine (Intermediate 37) and 4-(4,4,5,5-tetramethyl-1,3,2-dioxaborolan-2-yl)-1-((2-(trimethylsilyl)ethoxy)methyl)-1H-pyrazolo[3,4-b]pyridine (Intermediate 23) instead of 4-(4,4,5,5-tetramethyl-1,3,2-dioxaborolan-2-yl)-1-((2-(trimethylsilyl)ethoxy)methyl)-1H-pyrrolo[2,3-b]pyridine (Intermediate 21) in Step A. MS (ESI): mass calcd. for C₁₉H₁₇N₆O₂ 380.1; m/z found 381.1 [M+H]⁺. ¹H NMR (400 MHz, CD₃OD) δ 8.39 (d, J=4.8 Hz, 1H), 8.08 (d, J=2.9 Hz, 1H), 7.60 (dd, J=8.8, 4.4 Hz, 1H), 7.51 (s, 1H), 7.48 (s, 1H), 6.93 (d, J=4.8 Hz, 1H), 4.58 (d, J=5.3 Hz, 2H), 4.47 (d, J=5.6 Hz, 2H), 3.76 (s, 2H), 1.13 (s, 3H). Exchangeable protons not observed.

### Example 485: (*S)-2-(5-Fluoropyridin-2-yl)-7-methoxy-7-methyl-3-(1H-pyrazolo[3,4-b]pyridin-4-yl)-7,8-dihydro-4H,6H-pyrazolo[5,1-c][1,4]oxazepine

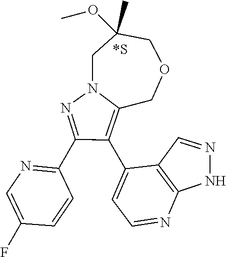 [SMILES: CO[C@]1(C)COCc2c(-c3ccnc4[nH]ncc34)c(-c3ccc(F)cn3)nn2C1]

Step A: 2-(5-Fluoropyridin-2-yl)-7-methoxy-7-methyl-3-(1-((2-(trimethylsilyl)ethoxy)methyl)-1H-pyrazolo[3,4-b]pyridin-4-yl)-7,8-dihydro-4H,6H-pyrazolo[5,1-c][1,4]oxazepane. To a solution of 2-(5-fluoropyridin-2-yl)-7-methyl-3-(1H-pyrazolo[3,4-b]pyridin-4-yl)-7,8-dihydro-4H,6H-pyrazolo[5,1-c][1,4]oxazepin-7-ol (Example 484, 43 mg, 0.084 mmol) in DMF (1.0 mL), cooled to 0° C., was added NaH (60% dispersion in mineral oil, 15 mg, 0.38 mmol), followed by Mel (23.7 μL, 0.38 mmol). The resulting mixture was stirred at 0° C. for 45 minutes and then diluted with EtOAc (5.0 mL) followed by water (5.0 mL). The layers were separated and the aqueous extracted with ethyl acetate (5 mL×2). The combined organics were dried over Na₂SO₄, filtered, and concentrated under reduced pressure to give the desired product (43.7 mg, 99%) as brownish gel which was used directly in Step B. MS (ESI): mass calcd. for C₂₆H₃₃FN₆O₃Si 524.2; m/z found 525.1 [M+H]⁺.

Step B: (*S)-2-(5-Fluoropyridin-2-yl)-7-methoxy-7-methyl-3-(1H-pyrazolo[3,4-b]pyridin-4-yl)-7,8-dihydro-4H,6H-pyrazolo[5,1-c][1,4]oxazepane. A solution of 2-(5-fluoropyridin-2-yl)-7-methoxy-7-methyl-3-(1-((2-(trimethylsilyl)ethoxy)methyl)-1H-pyrazolo[3,4-b]pyridin-4-yl)-7,8-dihydro-4H,6H-pyrazolo[5,1-c][1,4]oxazepine (43.7 mg, 0.0833 mmol) in trifluoroacetic acid (0.829 mL, 10.8 mmol) was stirred at room temperature for 2 hours. Solvent was evaporated and the residue was taken up in 2M NH₃ in MeOH (1.2 mL) and stirred at room temperature for 1.5 hours. The reaction mixture was concentrated under reduced pressure. The resulting residue was purified (basic ACCQ-prep. HPLC (20 mM NH₄OH in H₂O and neutral CH₃CN) to give the racemic mixture of the title compound (21.5 mg, 65%)). Purification by chiral SFC (Stationary phase: Reflect I Cellulose C 5 μm 250 mm×21 mm, Mobile phase: 20% methanol with 0.2% triethylamine, 80% CO₂. Flow rate 42 mL/min, monitor at 220 nm) afforded the title compound as a white solid (10 mg, 30.4%): MS (ESI): mass calcd. for C₂₀H₁₉FN₆O₂ 394.4; m/z found 395.1 [M+H]⁺; ¹H NMR (400 MHz, CD₃OD): δ 8.49 (d, J=4.8 Hz, 1H), 8.17 (dt, J=2.9, 0.7 Hz, 1H), 7.70 (ddd, J=8.9, 4.5, 0.7 Hz, 1H), 7.65-7.47 (m, 2H), 7.03 (d, J=4.8 Hz, 1H), 4.72 (dd, J=14.7, 1.9 Hz, 2H), 4.64 (dd, J=14.8, 2.3 Hz, 2H), 4.05 (dd, J=12.9, 1.8 Hz, 1H), 3.83 (d, J=12.9 Hz, 1H), 3.34 (s, 3H), 1.22 (s, 3H). N—H proton not observed; and (*R)-2-(5-Fluoropyridin-2-yl)-7-methoxy-7-methyl-3-(1H-pyrazolo[3,4-b]pyridin-4-yl)-7,8-dihydro-4H,6H-pyrazolo[5,1-c][1,4]oxazepane (Example 486).

### Example 486: (*R)-2-(5-Fluoropyridin-2-yl)-7-methoxy-7-methyl-3-(1H-pyrazolo[3,4-b]pyridin-4-yl)-7,8-dihydro-4H,6H-pyrazolo[5,1-c][1,4]oxazepine

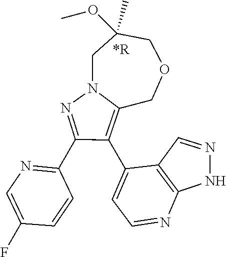 [SMILES: CO[C@@]1(C)COCc2c(-c3ccnc4[nH]ncc34)c(-c3ccc(F)cn3)nn2C1]

The title compound was isolated from chiral SFC purification of Example 485. MS (ESI): mass calcd. for C₂₀H₁₉FN₆O₂ 394.4; m/z found 395.1 [M+H]⁺. ¹H NMR (400 MHz, CD₃OD): δ 8.49 (d, J=4.8 Hz, 1H), 8.17 (dt, J=2.9, 0.7 Hz, 1H), 7.70 (ddd, J=8.9, 4.5, 0.7 Hz, 1H), 7.65-7.47 (m, 2H), 7.03 (d, J=4.8 Hz, 1H), 4.72 (dd, J=14.7, 1.9 Hz, 2H), 4.64 (dd, J=14.8, 2.3 Hz, 2H), 4.05 (dd, J=12.9, 1.8 Hz, 1H), 3.83 (d, J=12.9 Hz, 1H), 3.34 (s, 3H), 1.22 (s, 3H). N—H proton not observed.

Biological Data

Purified Enzyme Assay

In this assay, CSNK1D phosphorylates a substrate peptide PLSRTL-pS-VASLPGL in the presence of ATP. This substrate peptide has been modeled after the sequences surrounding three main cyclic AMP-dependent protein kinase sites of glycogen synthase. This assay monitors CSNK1D kinase activity by measuring the amount of ADP produced in the assay.

A substrate mix is prepared by diluting peptide substrate (final concentration 150 μM) with ATP (final concentration 20 μM) in assay buffer (50 mM Tris/HCl pH 7.4+10 mM MgCl₂+1 mM DTT+0.1% BSA). The substrate mix is added to each well of a low volume, 384-well, white opaque plate. Test compounds were diluted in HBSS and added in a dose-response to the plate.

To start the reaction, 2 nM of constitutively active human recombinant GST cleaved CSNK1D (University of Dundee, clone DU 19064, stored at 0.28 mg/mL in 50 mM Tris/HCl pH 7.5, 150 mM NaCl, 270 mM Sucrose, 0.1 mM EGTA, 0.1% 2-mercaptoethanol, 0.02% Brij-35.1 mM benzamidine, 0.2 mM PMSF) was added to each well and the plate centrifuged for 5 minutes at 1500 rpm. The total volume of each reaction is 5 ul (2 μL of substrate mix, 1 μL of diluted compounds, and 2 μL of human recombinant CSNK1D). The plates are incubated for 45 minutes at room temperature.

ADP was quantified using the ADP-Glo™ Kinase Assay. ADP-Glo Reagent (5 μL) was added to each well. After a 1 hour incubation at room temperature, Kinase Detection Reagent (10 μL) was added to each well and incubated for 30 minutes. Luminescence was measured on the Perkin Elmer Wallac EnVision 2104 Multi-label Reader. The raw data from the Envision is used to calculate percent activity. Percent activity is then graphed against the log of compound concentrations and these graphs are used to determine IC₅₀ of each compound.

Whole Cell nBRET CSNK1D Binding Assay

This cellular binding assay uses a bioluminescence resonance energy transfer to measure human CSNK1D binding activity in living Chinese Hamster Ovary (CHO) cells that are stably expressing human CSNK1D tagged with nanoluciferase. The cells were grown to confluency in growth media (DMEM:F12, 50 u/mLPen/Strep, 40 mM glutamine, and 0.6 mg/mL G418) in 10 cm² dishes. Cells were seeded onto white, opaque 384-well plates (Corning, cat #3704) at a density of 8,000 cells/well in serum-free OptiMEM and left to incubated overnight at 37° C., 5% CO₂. The next day, test compounds were added in a dose response to the plate followed by a NanoBret tracer (130 nM). The plate is mixed on an orbital shaker for 30 seconds, then placed in a 37 C incubator for 2 hours. NanoBret Nano-Glo Substrate solution (20 μL) is added to all wells and incubated for 3 minutes at room temperature. Donor (450 nm) and acceptor (630 nm) emissions are then measured within 10 minutes using a ClarioStar plate reader. The data is used to calculate millibret units, defined as (E630/E450)*1000. Bret emissions are then graphed against the log of compound concentrations and these graphs are used to determine the IC₅₀ of each compound.

TABLE 3

| | Whole Cell nBRET | ADP-Glo ™ |
|---|---|---|
| Example | CSNK1D Binding | CSNK1D Kinase |
| Number | Assay IC₅₀ (μM) | Assay IC₅₀ (μM) |
| 1 | 0.081 | 0.010 |
| 2 | 0.528 | 0.019 |
| 3 | 0.041 | 0.008 |
| 4 | 0.022 | 0.001 |
| 5 | 0.022 | 0.001 |
| 6 | 0.035 | 0.001 |
| 7 | 0.136 | 0.007 |
| 8 | 0.087 | 0.004 |
| 9 | 0.060 | 0.012 |
| 10 | 0.014 | 0.001 |
| 11 | 0.004 | 0.001 |
| 12 | 0.002 | 0.001 |
| 13 | 0.671 | 0.029 |
| 14 | 0.293 | 0.027 |
| 15 | 0.012 | 0.001 |
| 16 | 0.051 | 0.004 |
| 17 | 0.012 | 0.001 |
| 18 | 0.019 | 0.002 |
| 19 | 0.192 | 0.008 |
| 20 | 0.411 | 0.020 |
| 21 | 0.353 | 0.014 |
| 22 | 0.345 | 0.023 |
| 23 | 0.048 | 0.003 |
| 24 | 0.055 | 0.004 |
| 25 | 0.672 | 0.049 |
| 26 | 0.032 | 0.004 |
| 27 | 0.434 | 0.041 |
| 28 | 0.019 | 0.002 |
| 29 | 1.203 | 0.035 |
| 30 | 0.762 | 0.028 |
| 31 | 0.995 | 0.065 |
| 32 | 3.021 | 0.060 |
| 33 | 0.020 | 0.004 |
| 34 | 0.077 | 0.008 |
| 35 | 0.374 | 0.011 |
| 36 | 0.133 | 0.006 |
| 37 | 0.210 | 0.011 |
| 38 | 0.238 | 0.014 |
| 39 | 0.039 | 0.004 |
| 40 | 0.041 | 0.004 |
| 41 | 0.075 | 0.008 |
| 42 | 0.016 | 0.003 |
| 43 | 0.012 | 0.002 |
| 44 | 0.296 | 0.016 |
| 45 | 0.169 | 0.012 |
| 46 | 0.082 | 0.008 |
| 47 | 0.079 | 0.011 |
| 48 | 0.924 | 0.040 |
| 49 | 0.011 | 0.001 |
| 50 | 0.028 | 0.002 |
| 51 | 0.011 | 0.001 |
| 52 | 0.011 | 0.001 |
| 53 | 0.031 | 0.004 |
| 54 | 3.736 | 0.084 |
| 55 | 0.125 | 0.003 |
| 56 | 0.063 | 0.003 |
| 57 | 0.057 | 0.006 |
| 58 | 0.065 | 0.005 |
| 59 | 0.082 | 0.004 |
| 60 | 0.003 | 0.001 |
| 61 | 0.021 | 0.005 |
| 62 | 0.055 | 0.006 |
| 63 | 0.320 | 0.012 |
| 64 | 0.012 | 0.001 |
| 65 | 0.055 | 0.003 |
| 66 | 0.004 | 0.002 |
| 67 | 0.243 | 0.020 |
| 68 | 0.206 | 0.027 |
| 69 | 0.043 | 0.007 |
| 70 | 0.037 | 0.002 |
| 71 | 0.021 | 0.002 |
| 72 | 0.482 | 0.054 |
| 73 | 0.569 | 0.120 |
| 74 | 2.406 | 0.205 |
| 75 | >10 | 0.015 |
| 76 | 2.926 | 0.168 |
| 77 | 2.325 | 0.116 |
| 78 | 0.337 | 0.015 |
| 79 | 0.227 | 0.007 |
| 80 | 3.056 | 0.018 |
| 81 | 0.048 | 0.002 |
| 82 | 0.026 | 0.002 |
| 83 | 0.034 | 0.003 |
| 84 | 0.025 | 0.001 |
| 85 | 0.371 | 0.022 |
| 86 | 0.254 | 0.029 |
| 87 | 0.162 | 0.013 |
| 88 | 0.248 | 0.012 |
| 89 | 0.275 | 0.009 |
| 90 | 0.352 | 0.016 |
| 91 | 0.151 | 0.008 |
| 92 | 0.100 | 0.004 |
| 93 | 0.297 | 0.010 |
| 94 | 0.092 | 0.007 |
| 95 | 0.557 | 0.019 |
| 96 | 2.727 | 0.032 |
| 97 | 5.048 | 0.063 |
| 98 | 2.281 | 0.031 |
| 99 | 0.054 | NT |
| 100 | 0.697 | 0.055 |
| 101 | 0.024 | 0.002 |
| 102 | 0.844 | 0.039 |
| 103 | 0.015 | NT |
| 104 | 0.049 | 0.004 |
| 105 | 0.046 | 0.004 |
| 106 | 0.137 | 0.009 |
| 107 | 0.072 | 0.009 |
| 108 | 0.681 | 0.041 |
| 109 | 0.301 | 0.015 |
| 110 | 1.169 | 0.049 |
| 111 | 1.066 | 0.036 |
| 112 | 0.054 | 0.003 |
| 113 | 0.113 | 0.010 |
| 114 | 0.055 | 0.006 |
| 115 | 0.074 | 0.006 |
| 116 | 0.926 | 0.038 |
| 117 | 0.925 | 0.045 |
| 118 | 1.164 | 0.049 |
| 119 | 0.751 | 0.032 |
| 120 | 0.201 | 0.013 |
| 121 | 0.233 | 0.014 |
| 122 | 3.406 | 0.120 |
| 123 | 2.350 | 0.111 |
| 124 | 0.343 | 0.017 |
| 125 | 0.270 | 0.012 |
| 126 | 0.827 | 0.021 |
| 127 | 3.248 | 0.051 |
| 128 | 0.244 | 0.013 |
| 129 | 0.440 | 0.036 |
| 130 | 0.624 | 0.031 |
| 131 | 0.016 | 0.003 |
| 132 | 0.011 | 0.003 |
| 133 | 0.008 | 0.002 |
| 134 | 0.007 | 0.001 |
| 135 | 0.006 | 0.003 |
| 136 | 0.006 | 0.002 |
| 137 | 1.326 | 0.054 |
| 138 | 0.999 | 0.106 |
| 139 | >10 | 0.450 |
| 140 | 0.122 | 0.018 |
| 141 | 5.690 | 0.344 |
| 142 | 0.070 | 0.004 |
| 143 | 0.038 | 0.005 |
| 144 | 0.003 | 0.001 |
| 145 | 0.004 | 0.001 |
| 146 | 0.359 | 0.018 |
| 147 | 0.011 | 0.001 |
| 148 | 3.285 | 0.059 |
| 149 | >10 | 1.424 |
| 150 | 0.058 | 0.002 |
| 151 | 0.042 | 0.003 |
| 152 | 0.025 | 0.003 |
| 153 | 3.705 | 0.176 |
| 154 | 0.023 | 0.002 |
| 155 | 0.172 | 0.008 |
| 156 | 0.087 | 0.005 |
| 157 | 0.072 | 0.008 |
| 158 | >10 | 6.874 |
| 159 | >10 | 1.184 |
| 160 | 0.143 | 0.009 |
| 161 | 1.400 | 0.026 |
| 162 | >10 | 0.143 |
| 163 | 1.919 | 0.059 |
| 164 | 0.165 | 0.009 |
| 165 | 0.702 | 0.015 |
| 166 | 0.102 | 0.007 |
| 167 | 0.091 | 0.003 |
| 168 | 0.836 | 0.019 |
| 169 | 0.134 | 0.004 |
| 170 | 0.042 | 0.002 |
| 171 | 0.006 | 0.001 |
| 172 | 0.054 | 0.005 |
| 173 | 0.840 | 0.042 |
| 174 | 0.114 | 0.008 |
| 175 | 3.613 | 0.148 |
| 176 | 0.198 | 0.007 |
| 177 | 0.044 | 0.003 |
| 178 | 1.004 | 0.077 |
| 179 | 0.015 | 0.003 |
| 180 | 0.252 | 0.054 |
| 181 | 3.242 | 0.032 |
| 182 | >10 | 0.241 |
| 183 | 0.220 | 0.007 |
| 184 | 0.015 | 0.002 |
| 185 | 0.017 | 0.001 |
| 186 | 0.257 | 0.009 |
| 187 | 0.330 | 0.008 |
| 188 | 0.139 | 0.012 |
| 189 | 0.320 | 0.010 |
| 190 | 0.215 | 0.004 |
| 191 | 0.005 | <0.0005 |
| 192 | 0.004 | 0.001 |
| 193 | 0.005 | <0.0005 |
| 194 | 0.011 | 0.001 |
| 195 | 0.218 | 0.021 |
| 196 | 0.040 | 0.002 |
| 197 | 0.038 | 0.003 |
| 198 | 0.059 | 0.003 |
| 199 | 0.186 | 0.012 |
| 200 | 0.056 | 0.004 |
| 201 | 0.246 | 0.017 |
| 202 | 0.198 | 0.010 |
| 203 | 2.891 | 0.034 |
| 204 | 0.305 | 0.013 |
| 205 | 0.171 | 0.005 |
| 206 | 0.249 | 0.014 |
| 207 | 1.773 | 0.051 |
| 208 | 0.821 | 0.026 |
| 209 | 0.165 | 0.008 |
| 210 | 0.023 | <0.0005 |
| 211 | 0.006 | 0.001 |
| 212 | 0.001 | <0.0005 |
| 213 | 0.002 | 0.001 |
| 214 | 0.075 | 0.009 |
| 215 | 0.028 | 0.001 |
| 216 | 0.038 | 0.002 |
| 217 | 0.025 | 0.002 |
| 218 | 0.239 | 0.012 |
| 219 | 0.279 | 0.038 |
| 220 | 1.495 | 0.099 |
| 221 | 0.460 | 0.007 |
| 222 | 0.181 | 0.011 |
| 223 | 0.894 | 0.022 |
| 224 | 0.005 | 0.004 |
| 225 | 0.088 | 0.008 |
| 226 | 0.048 | 0.010 |
| 227 | 0.003 | 0.001 |
| 228 | 0.079 | 0.007 |
| 229 | 0.002 | 0.001 |
| 230 | 0.074 | 0.005 |
| 231 | 0.004 | 0.001 |
| 232 | 0.003 | 0.001 |
| 233 | 0.007 | 0.002 |
| 234 | 0.032 | 0.002 |
| 235 | 0.008 | 0.001 |
| 236 | 0.021 | 0.003 |
| 237 | 0.013 | 0.004 |
| 238 | 0.023 | 0.002 |
| 239 | 0.115 | 0.004 |
| 240 | 0.019 | 0.002 |
| 241 | 0.007 | 0.002 |
| 242 | 0.004 | 0.002 |
| 243 | 0.049 | 0.003 |
| 244 | 0.029 | 0.004 |
| 245 | 0.043 | 0.005 |
| 246 | 0.058 | 0.005 |
| 247 | 0.013 | 0.002 |
| 248 | 0.006 | 0.002 |
| 249 | 0.197 | 0.017 |
| 250 | 0.005 | 0.001 |
| 251 | 0.185 | 0.026 |
| 252 | 0.003 | 0.002 |
| 253 | 0.279 | 0.021 |
| 254 | 0.085 | 0.004 |
| 255 | 1.952 | 0.066 |
| 256 | 0.117 | 0.012 |
| 257 | 10.790 | 0.197 |
| 258 | 0.071 | 0.006 |
| 259 | 1.919 | 0.079 |
| 260 | 0.107 | 0.003 |
| 261 | 2.665 | 0.063 |
| 262 | 0.015 | 0.005 |
| 263 | 1.464 | 0.054 |
| 264 | 0.020 | 0.004 |
| 265 | 0.037 | 0.010 |
| 266 | 0.337 | 0.052 |
| 267 | 0.131 | 0.006 |
| 268 | 0.005 | 0.001 |
| 269 | 0.073 | 0.006 |
| 270 | 0.008 | 0.001 |
| 271 | 0.004 | 0.002 |
| 272 | 0.071 | 0.002 |
| 273 | 0.029 | 0.002 |
| 274 | 0.056 | 0.005 |
| 275 | 0.009 | 0.001 |
| 276 | 0.006 | 0.001 |
| 277 | 0.043 | 0.006 |
| 278 | 0.064 | 0.007 |
| 279 | 0.074 | 0.005 |
| 280 | 0.019 | 0.004 |
| 281 | 0.054 | 0.004 |
| 282 | 0.048 | 0.009 |
| 283 | 0.007 | 0.002 |
| 284 | 0.068 | 0.006 |
| 285 | 0.100 | 0.011 |
| 286 | 0.081 | 0.004 |
| 287 | 0.068 | 0.005 |
| 288 | 4.099 | 0.238 |
| 289 | 0.114 | 0.094 |
| 290 | 0.120 | 0.539 |
| 291 | 0.006 | 0.005 |
| 292 | 0.021 | 0.003 |
| 293 | 0.018 | 0.002 |
| 294 | 0.023 | 0.002 |
| 295 | 0.024 | 0.002 |
| 296 | 0.240 | 0.017 |
| 297 | 0.297 | 0.012 |
| 298 | 0.012 | 0.002 |
| 299 | 0.303 | 0.020 |
| 300 | 1.908 | 0.075 |
| 301 | 0.110 | 0.004 |
| 302 | 3.248 | 0.132 |
| 303 | 0.180 | 0.009 |
| 304 | 0.034 | 0.004 |
| 305 | 2.846 | 0.089 |
| 306 | 0.068 | 0.008 |
| 307 | >10 | 0.183 |
| 308 | 0.034 | 0.006 |
| 309 | 1.317 | 0.097 |
| 310 | 0.034 | 0.003 |
| 311 | 1.407 | 0.092 |
| 312 | 0.039 | 0.004 |
| 313 | 5.125 | 0.102 |
| 314 | 0.014 | 0.004 |
| 315 | 1.878 | 0.073 |
| 316 | 0.054 | 0.006 |
| 317 | 8.519 | 0.111 |
| 318 | 0.040 | 0.003 |
| 319 | 0.081 | 0.005 |
| 320 | 0.105 | 0.014 |
| 321 | 0.037 | NT |
| 322 | NT | NT |
| 323 | 0.015 | 0.001 |
| 324 | 0.112 | 0.037 |
| 325 | 0.001 | 0.001 |
| 326 | 0.023 | 0.006 |
| 327 | 0.097 | 0.014 |
| 328 | 0.008 | 0.001 |
| 329 | 0.163 | 0.023 |
| 330 | 0.022 | 0.002 |
| 331 | 0.089 | 0.018 |
| 332 | 0.012 | 0.001 |
| 333 | >10 | 4.959 |
| 334 | 1.555 | 0.070 |
| 335 | 0.373 | 0.013 |
| 336 | 0.009 | 0.001 |
| 337 | 0.080 | 0.003 |
| 338 | 0.072 | 0.005 |
| 339 | 0.007 | 0.001 |
| 340 | 0.039 | 0.005 |
| 341 | 0.024 | 0.002 |
| 342 | 0.454 | 0.020 |
| 343 | 0.096 | 0.005 |
| 344 | 0.044 | 0.004 |
| 345 | 0.368 | 0.034 |
| 346 | 0.173 | 0.025 |
| 347 | 0.573 | 0.035 |
| 348 | 0.003 | 0.001 |
| 349 | 0.074 | 0.008 |
| 350 | 0.001 | 0.001 |
| 351 | 0.020 | 0.002 |
| 352 | 1.301 | 0.050 |
| 353 | 3.833 | 1.116 |
| 354 | 0.071 | 0.018 |
| 355 | 0.027 | 0.009 |
| 356 | 0.002 | 0.001 |
| 357 | 0.040 | 0.008 |
| 358 | 0.085 | 0.004 |
| 359 | >10 | 1.674 |
| 360 | 0.070 | 0.007 |
| 361 | 0.349 | 0.497 |
| 362 | 0.003 | 0.001 |
| 363 | 1.406 | 0.060 |
| 364 | 0.102 | 0.005 |
| 365 | 0.008 | 0.001 |
| 366 | 0.176 | 0.014 |
| 367 | 0.566 | 0.041 |
| 368 | 0.055 | 0.007 |
| 369 | 0.096 | 0.012 |
| 370 | >10 | 0.524 |
| 371 | 7.800 | 0.404 |
| 372 | 0.724 | 0.042 |
| 373 | >10 | 0.306 |
| 374 | >10 | 0.293 |
| 375 | 0.109 | 0.065 |
| 376 | 0.074 | 0.004 |
| 377 | 2.478 | 0.046 |
| 378 | 2.298 | 0.119 |
| 379 | 0.738 | 0.041 |
| 380 | 0.093 | 0.010 |
| 381 | 0.228 | 0.064 |
| 382 | >10 | 0.451 |
| 383 | 1.036 | 0.020 |
| 384 | 0.171 | 0.015 |
| 385 | 1.574 | 0.050 |
| 386 | 9.656 | 0.162 |
| 387 | 0.760 | 0.030 |
| 388 | 0.014 | 0.004 |
| 389 | 0.094 | 0.005 |
| 390 | 0.141 | 0.006 |
| 391 | 5.378 | 0.258 |
| 392 | 0.026 | 0.007 |
| 393 | 2.739 | 0.049 |
| 394 | 0.018 | 0.002 |
| 395 | 0.021 | 0.002 |
| 396 | 0.292 | 0.022 |
| 397 | 0.011 | 0.002 |
| 398 | 0.029 | 0.003 |
| 399 | 0.095 | 0.005 |
| 400 | 0.030 | 0.004 |
| 401 | 0.043 | 0.004 |
| 402 | 0.075 | 0.005 |
| 403 | 0.023 | 0.005 |
| 404 | 0.137 | 0.009 |
| 405 | 0.014 | 0.005 |
| 406 | 0.023 | 0.003 |
| 407 | 0.219 | 0.025 |
| 408 | 0.026 | 0.006 |
| 409 | 0.069 | 0.012 |
| 410 | 0.017 | 0.002 |
| 411 | 0.040 | 0.003 |
| 412 | 0.694 | 0.149 |
| 413 | 0.075 | 0.027 |
| 414 | 0.330 | 0.008 |
| 415 | 3.888 | 0.264 |
| 416 | 0.118 | 0.017 |
| 417 | 0.003 | 0.001 |
| 418 | 0.124 | 0.016 |
| 419 | >10 | 0.235 |
| 420 | 0.024 | 0.003 |
| 421 | 0.682 | 0.036 |
| 422 | 0.020 | 0.003 |
| 423 | 1.567 | 0.067 |
| 424 | 0.040 | 0.004 |
| 425 | 2.257 | 0.235 |
| 426 | 0.087 | 0.017 |
| 427 | 0.019 | 0.004 |
| 428 | 0.076 | 0.009 |
| 429 | 2.234 | 0.133 |
| 430 | 1.187 | 0.092 |
| 431 | >10 | 1.583 |
| 432 | 0.195 | 0.008 |
| 433 | 0.079 | 0.004 |
| 434 | 8.289 | 0.165 |
| 435 | 0.049 | 0.005 |
| 436 | 3.961 | 0.156 |
| 437 | 0.046 | 0.005 |
| 438 | 0.113 | 0.012 |
| 439 | 0.075 | 0.006 |
| 440 | 0.516 | 0.044 |
| 441 | 1.871 | 0.044 |
| 442 | 0.023 | 0.003 |
| 443 | 0.581 | 0.030 |
| 444 | 1.588 | 0.067 |
| 445 | 0.210 | 0.010 |
| 446 | 4.340 | 0.184 |
| 447 | 0.005 | 0.002 |
| 448 | 0.196 | 0.028 |
| 449 | 0.070 | 0.008 |
| 450 | 1.050 | 0.075 |
| 451 | 5.765 | 0.132 |
| 452 | 0.570 | 0.044 |
| 453 | 0.011 | 0.002 |
| 454 | 0.127 | 0.006 |
| 455 | 0.001 | 0.001 |
| 456 | 0.007 | 0.001 |
| 457 | 0.002 | 0.001 |
| 458 | 0.007 | 0.001 |
| 459 | 0.080 | 0.004 |
| 460 | 0.024 | 0.002 |
| 461 | 0.002 | 0.001 |
| 462 | 0.020 | 0.001 |
| 463 | 0.020 | 0.002 |
| 464 | 0.011 | 0.005 |
| 465 | 0.035 | 0.003 |
| 466 | 0.024 | 0.012 |
| 467 | 0.021 | 0.002 |
| 468 | 0.187 | 0.025 |
| 469 | 0.385 | 0.028 |
| 470 | 0.017 | 0.001 |
| 471 | 0.003 | 0.002 |
| 472 | 0.597 | 0.069 |
| 473 | 0.006 | 0.002 |
| 474 | 0.002 | 0.001 |
| 475 | 0.036 | 0.006 |
| 476 | 0.001 | 0.001 |
| 477 | 0.033 | 0.006 |
| 478 | 0.004 | 0.001 |
| 479 | 1.325 | 0.032 |
| 480 | 0.228 | 0.010 |
| 481 | 0.065 | 0.006 |
| 482 | 0.079 | 0.008 |
| 483 | 0.048 | 0.005 |
| 484 | >10 | 0.231 |
| 485 | 8.056 | 0.370 |
| 486 | >10 | 0.763 |

NT means Not tested.

1. A compound of Formula (I), 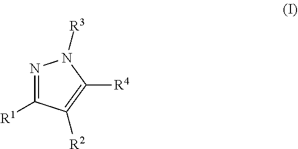
wherein
R¹ is selected from the group consisting of:
(a) phenyl substituted with one or two halo members;
(b) 5-fluoro-2-pyridyl optionally substituted with halo or C₁₋₃alkyl, 5-fluoro-3-pyridyl, 5-chloropyridin-2-yl, 3-chloropyridin-4-yl, 6-methoxypyridin-2-yl, 5-chloro-6-methylpyridin-2-yl, or 3,5-difluoropyridin-4-yl; and
(c) oxazol-5-yl, thiazol-2-yl, thiazol-4-yl, or 1-methyl-1H-imidazol-4-yl;
R² is selected from the group consisting of:
(d) 4-pyridyl optionally substituted with one member selected from the group consisting of: halo, C₁₋₃haloalkyl, CH₂OH, OC₁₋₃alkyl, (C═O)—NHCH₃, NH₂, NH—(C═O)C₁₋₃alkyl, NH—(C═O)C₁₋₃haloalkyl, NH—(C═O)phenyl, NH—(C═O)cyclopropyl, and NH—(C═O)cyclopropyl wherein the cyclopropyl is substituted with one or two halo; 2,5-difluoro-4-pyridyl; 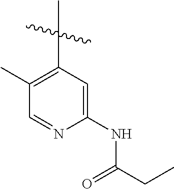 [SMILES: *c1cc(NC(=O)CC)ncc1C]
or 5-methylpyridin-2-amine;
(e) fused heteroaryl selected from the group consisting of: thieno[3,2-b]pyridinyl; pyrazolo[1,5-a]pyridin-5-yl optionally substituted with halo, C₁₋₃alkyl, or NH₂; 1H-pyrrolo[3,2-b]pyridinyl; 1H-pyrrolo[2,3-b]pyridinyl optionally substituted with one two or three members each independently selected from the group consisting of: halo, C₁₋₃alkyl, C₁₋₃haloalkyl, and CN; pyrazolo[1,5-a]pyrimidin-5-yl; [1,2,4]triazolo[1,5-a]pyridin-7-yl optionally substituted with C₁₋₃alkyl; 1H-pyrazolo[4,3-b]pyridin-7-yl optionally substituted with C₁₋₃alkyl; 1H-pyrazolo[3,4-b]pyridin-5-yl optionally substituted with C₁₋₃alkyl; 1H-pyrazolo[3,4-b]pyridinyl optionally substituted with one, two or three members each independently selected from the group consisting of: halo, C₁₋₃alkyl, C₁₋₃haloalkyl, and cyclopropyl; 2-methyl-2H-pyrazolo[4,3-b]pyridin-7-yl; 1-methyl-1H-pyrazolo[4,3-b]pyridin-7-yl; 2-methylpyrazolo[3,4-b]pyridin-4-yl; or 1H-pyrazolo[3,4-d]pyrimidin-4-yl optionally substituted with C₁₋₃alkyl; and
(f) 1,5-naphthyridin-4-yl optionally substituted with halo or OC₁₋₃alkyl;
R³ and R⁴ come together to form a group selected from the group consisting of: 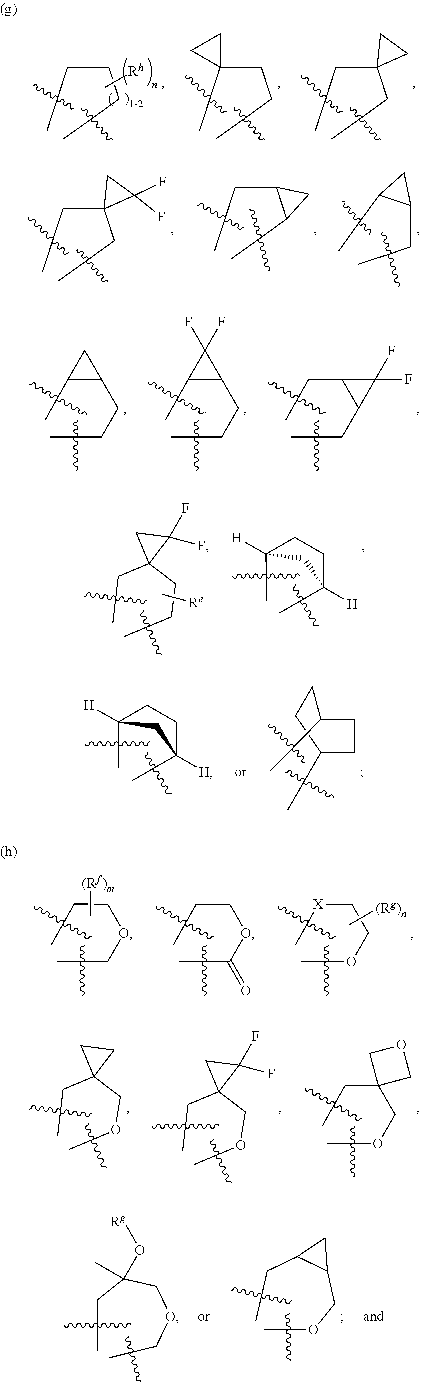 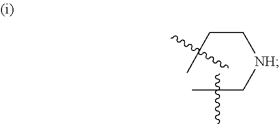 [SMILES: *CCNC*]
R^{f} is independently selected from the group consisting of: H, C₁₋₃alkyl, C₁₋₃haloalkyl, cyclopropyl, cyclobutyl, and two R^{f} members come together to form a C₃₋₆cycloalkyl wherein the C₃₋₆cycloalkyl is optionally substituted with one or two halo members;
R^{g} is H, halo, or C₁₋₃alkyl;
R^{h} is independently selected from the group consisting of H, halo, OH, C₁₋₃alkyl, CH₂OCH₃, CH₂CH₂OCH₃, C₁₋₃haloalkyl, CH₂OCHF₂, CH₂OCF₃, CN, and 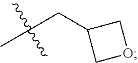 [SMILES: *CC1COC1]
X is selected from the group consisting of: a bond, CH₂, CH(CH₃), and CH₂CH₂;
m is 1, 2, 3 or 4; and
n is 1, 2, or 3;
and pharmaceutically acceptable salts, isotopes, N-oxides, solvates, and stereoisomers of compounds of Formula (I).

2. The compound of claim 1, wherein R¹ is

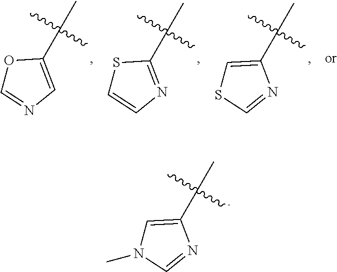

3. The compound of claim 1, wherein R¹ is

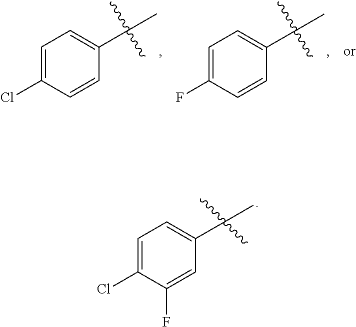 [SMILES: *c1ccc(Cl)cc1 ; *c1ccc(F)cc1 ; *c1ccc(Cl)c(F)c1]

4. The compound of claim 1, wherein R¹ is

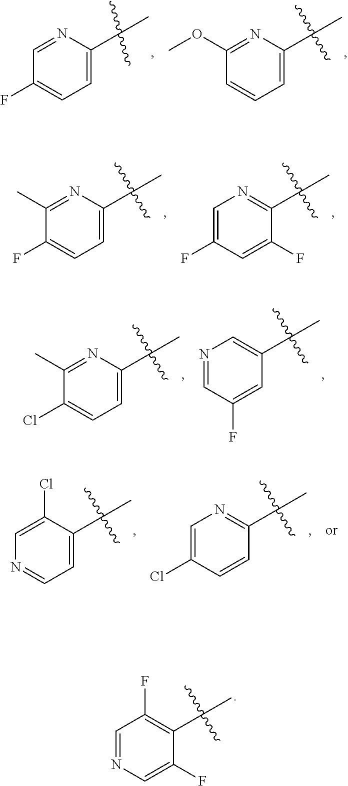

5. The compound of claim 1, wherein R¹ is

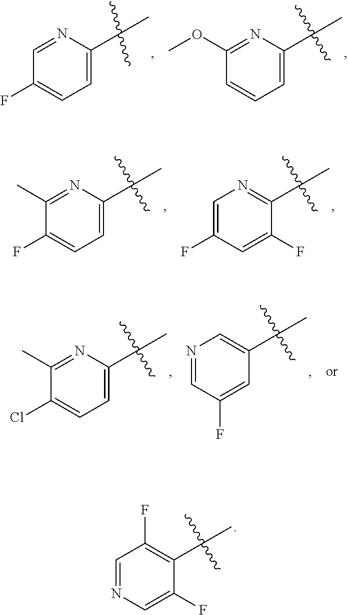

6. The compound of claim 1, wherein R¹ is

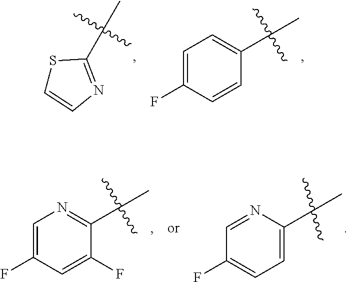 [SMILES: *c1nccs1 ; *c1ccc(F)cc1 ; *c1ncc(F)cc1F ; *c1ccc(F)cn1]

7. The compound of claim 1, wherein R² is

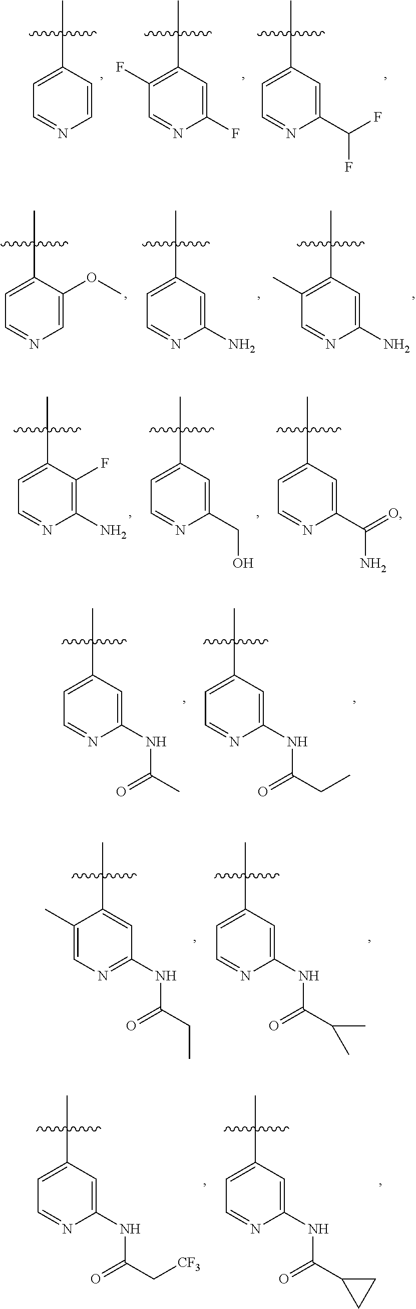

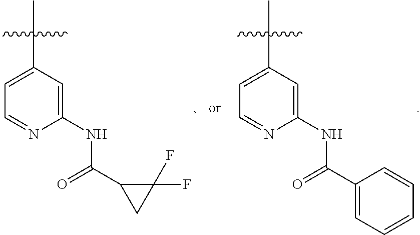 [SMILES: *c1ccnc(NC(=O)C2CC2(F)F)c1 ; *c1ccnc(NC(=O)c2ccccc2)c1]

8. The compound of claim 1, wherein R² is

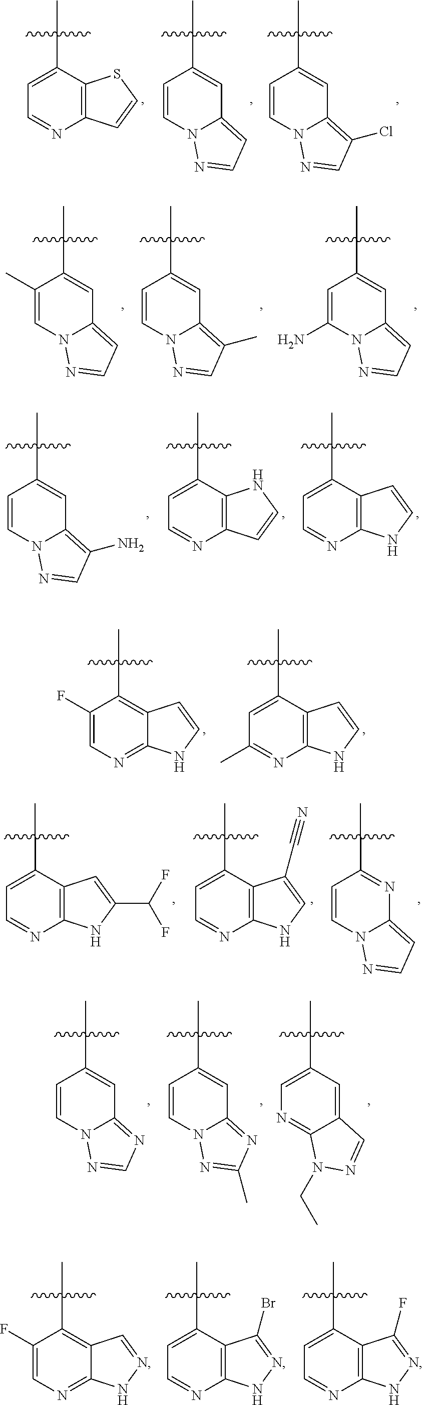

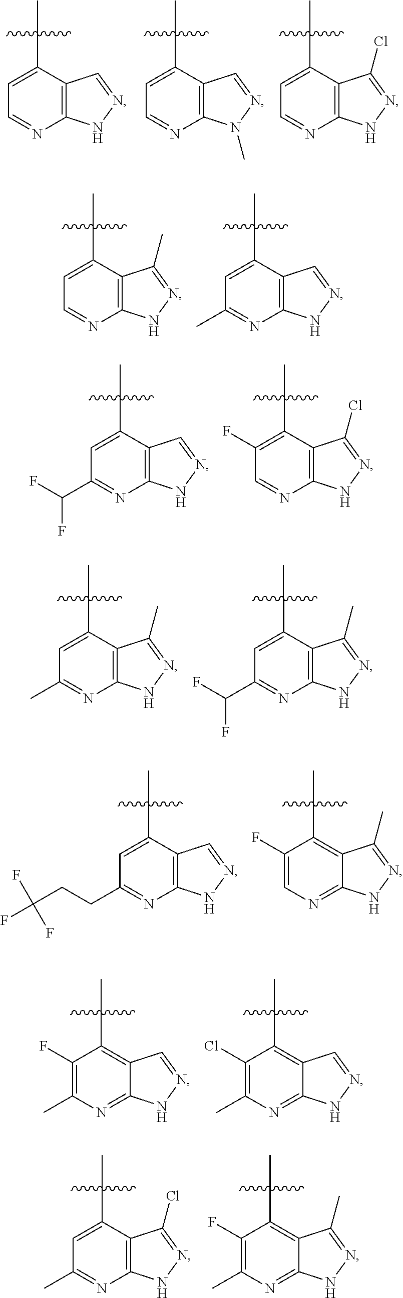

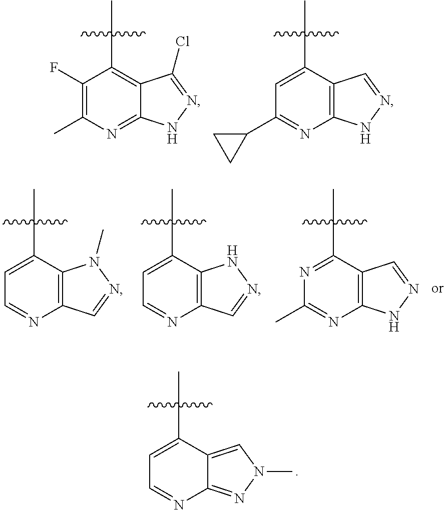

9. The compound of claim 1, wherein R² is

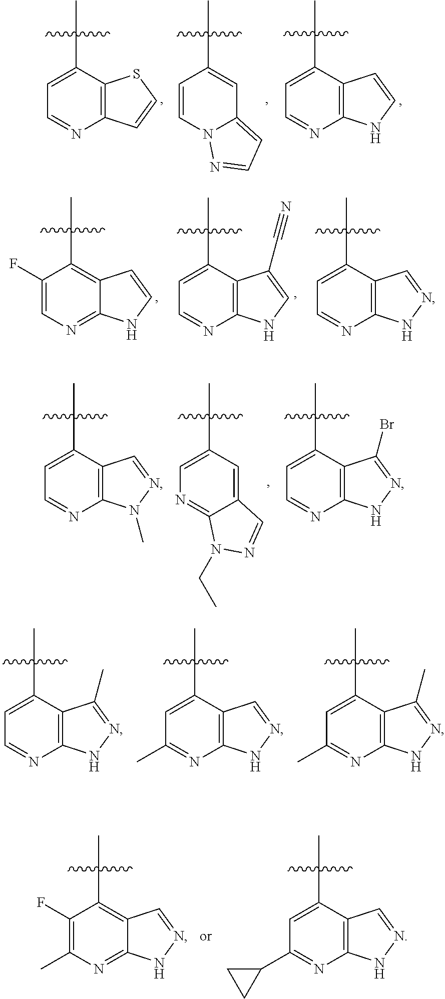

10. The compound of claim 1, wherein R² is

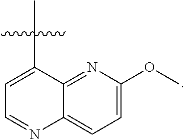 [SMILES: *c1ccnc2ccc(OC)nc12]

11. The compound of claim 1, wherein R² is

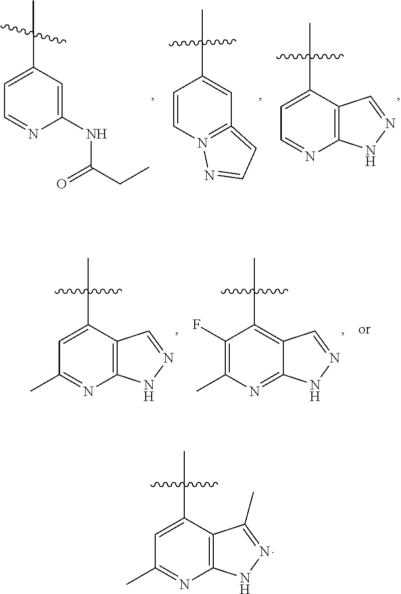

12. The compound of claim 1, wherein R³ and R⁴ is

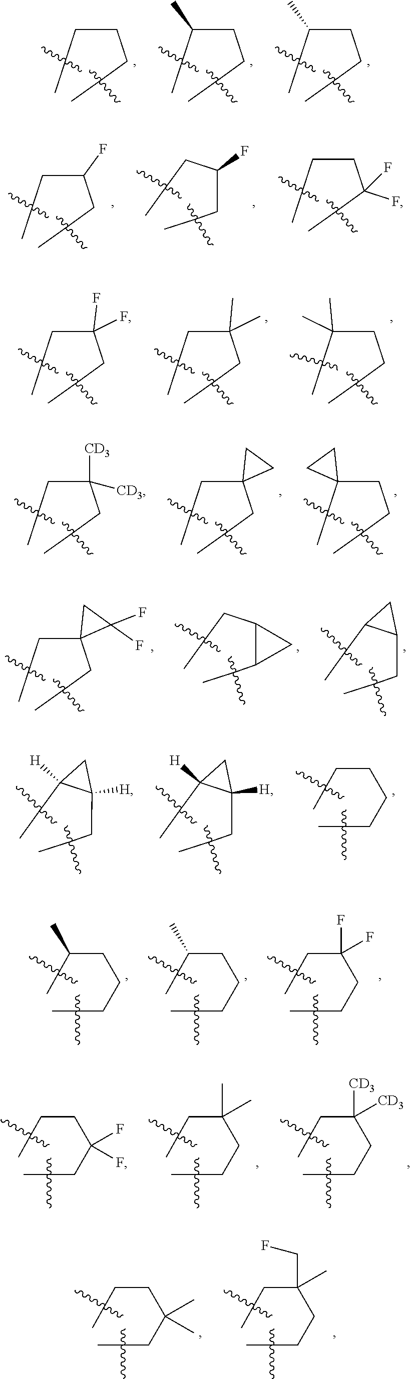

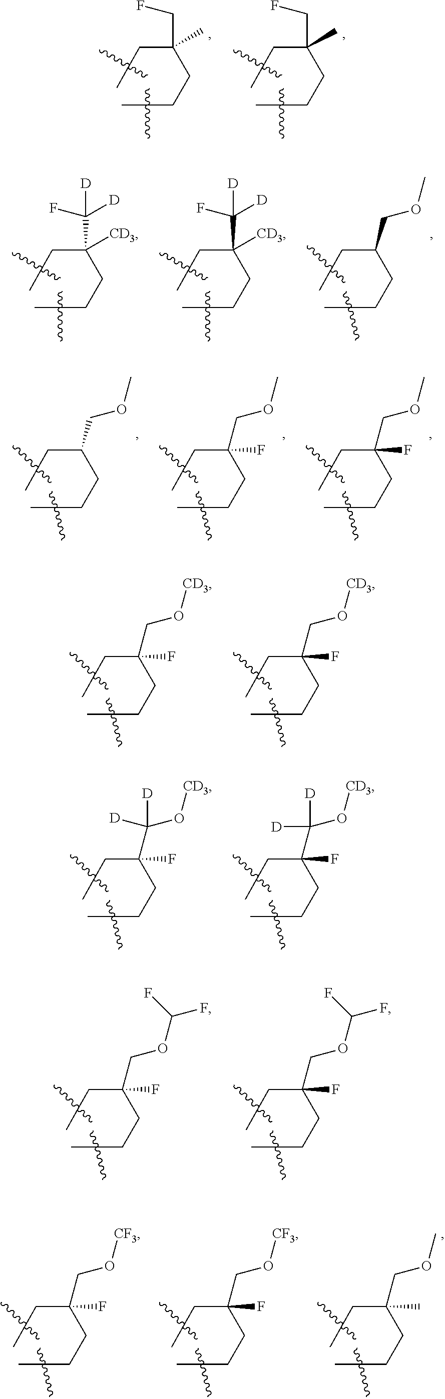

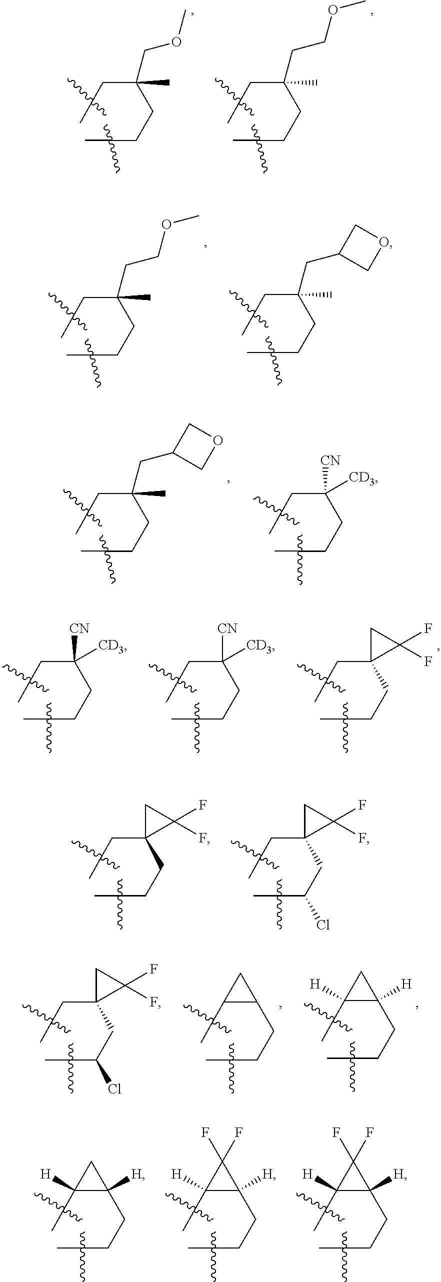

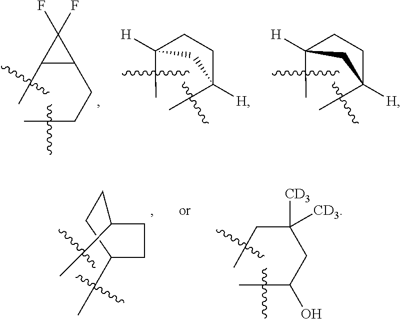

13. The compound of claim 1, wherein R³ and R⁴ is

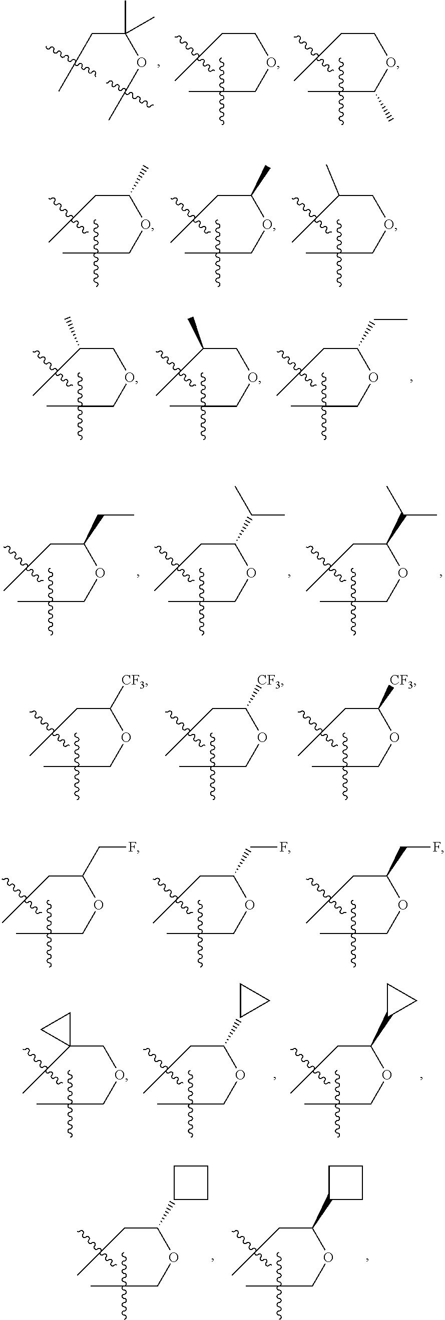

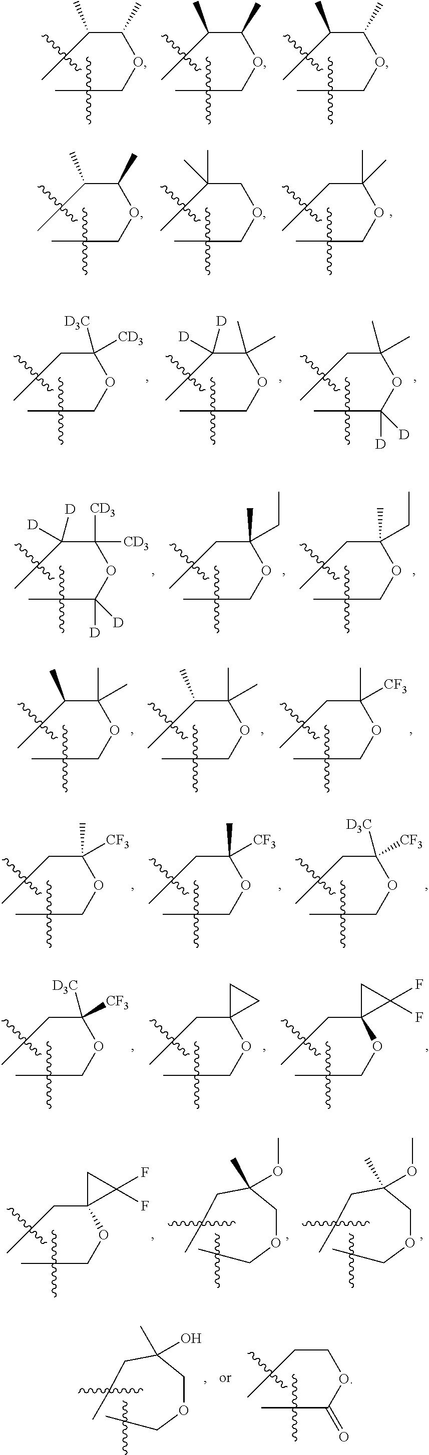

14. The compound of claim 1, wherein R³ and R⁴ is

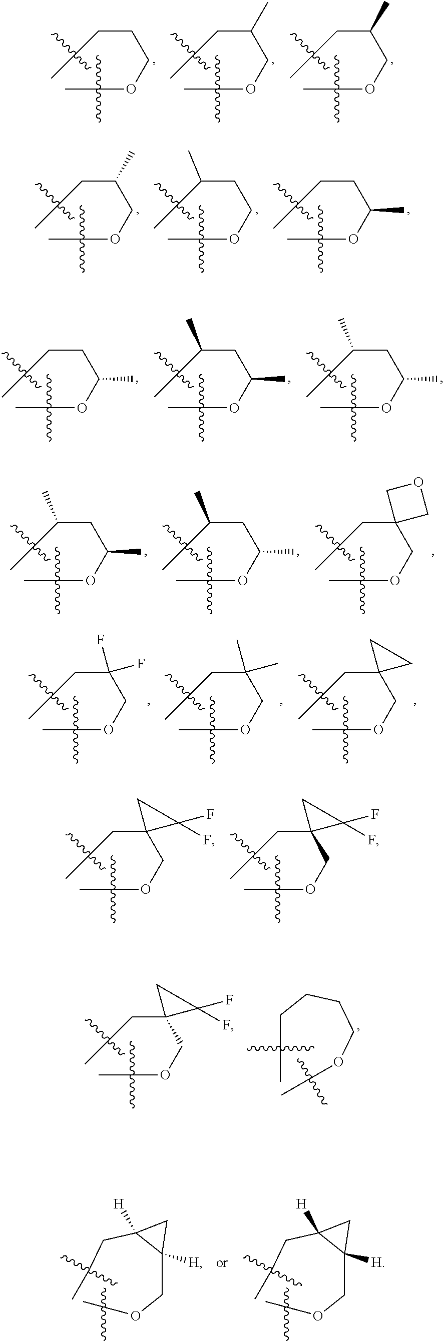

15. The compound of claim 1, wherein R³ and R⁴ is

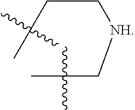 [SMILES: *CCNC*]

16. The compound of claim 1, wherein R³ and R⁴ is

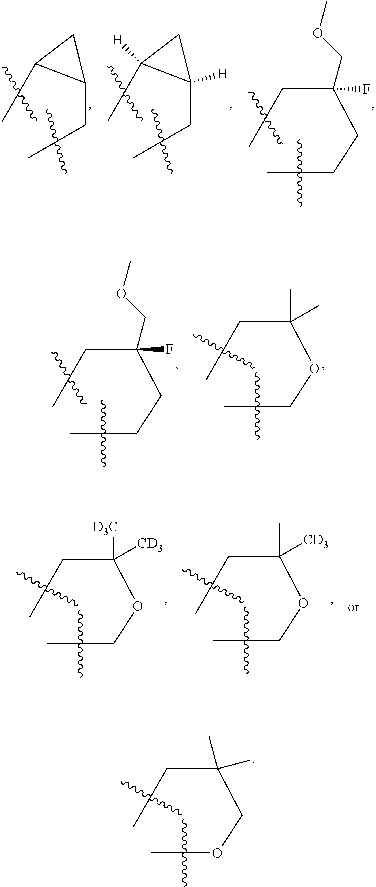

17. (canceled)

18. (canceled)

19. (canceled)

20. (canceled)

21. (canceled)

22. (canceled)

23. (canceled)

24. The compound of claim 1, wherein the compound is
2-(5-Fluoro-2-pyridyl)-3-(1H-pyrrolo[2,3-b]pyridin-4-yl)-6,7-dihydro-4H-pyrazolo[5,1-c][1,4]oxazine;
4-[2-(5-Fluoro-2-pyridyl)-4,5,6,7-tetrahydropyrazolo[1,5-a]pyrazin-3-yl]-6-methyl-1H-pyrazolo[3,4-b]pyridine;
4-[2-(5-Fluoro-2-pyridyl)-4,5,6,7-tetrahydropyrazolo[1,5-a]pyridin-3-yl]-1H-pyrrolo[2,3-b]pyridine;
4-(2-(4-Fluorophenyl)-5,6-dihydro-4H-pyrrolo[1,2-b]pyrazol-3-yl)-1H-pyrazolo[3,4-b]pyridine;
4-(2-(4-Fluorophenyl)-5,6-dihydro-4H-pyrrolo[1,2-b]pyrazol-3-yl)-6-methyl-1H-pyrazolo[3,4-b]pyridine;
4-(2-(5-Fluoropyridin-2-yl)-5, 6-dihydro-4H-pyrrolo[1,2-b]pyrazol-3-yl)-1H-pyrrolo[2,3-b]pyridine;
4-[2-(5-Fluoro-2-pyridyl)-5,6-dihydro-4H-pyrrolo[1,2-b]pyrazol-3-yl]-1H-pyrazolo[3,4-b]pyridine;
4-[2-(5-Fluoro-2-pyridyl)-5,6-dihydro-4H-pyrrolo[1,2-b]pyrazol-3-yl]-6-methyl-1H-pyrazolo[3,4-b]pyridine;
5-Fluoro-4-(2-(5-fluoropyridin-2-yl)-5,6-dihydro-4H-pyrrolo[1,2-b]pyrazol-3-yl)-6-methyl-1H-pyrazolo[3,4-b]pyridine;
(S)-4-(5-Fluoro-2-(4-fluorophenyl)-5,6-dihydro-4H-pyrrolo[1,2-b]pyrazol-3-yl)pyridin-2-amine;
(S)-4-(5-Fluoro-2-(4-fluorophenyl)-5,6-dihydro-4H-pyrrolo[1,2-b]pyrazol-3-yl)-1H-pyrazolo[3,4-b]pyridine;
(S)-4-(5-Fluoro-2-(4-fluorophenyl)-5,6-dihydro-4H-pyrrolo[1,2-b]pyrazol-3-yl)-6-methyl-1H-pyrazolo[3,4-b]pyridine;
4-(5-Fluoro-2-(5-fluoropyridin-2-yl)-5,6-dihydro-4H-pyrrolo[1,2-b]pyrazol-3-yl)-6-methyl-1H-pyrazolo[3,4-b]pyridine;
4-(4,4-Difluoro-2-(5-fluoropyridin-2-yl)-5,6-dihydro-4H-pyrrolo[1,2-b]pyrazol-3-yl)-1H-pyrazolo[3,4-b]pyridine;
4-[(6S)-2-(4-Fluorophenyl)-6-methyl-5,6-dihydro-4H-pyrrolo[1,2-b]pyrazol-3-yl]-1H-pyrazolo[3,4-b]pyridine;
4-[(6R)-2-(4-Fluorophenyl)-6-methyl-5, 6-dihydro-4H-pyrrolo[1,2-b]pyrazol-3-yl]-1H-pyrazolo[3,4-b]pyridine;
4-[(6S)-2-(4-Fluorophenyl)-6-methyl-5,6-dihydro-4H-pyrrolo[1,2-b]pyrazol-3-yl]-6-methyl-1H-pyrazolo[3,4-b]pyridine;
4-[(6R)-2-(4-Fluorophenyl)-6-methyl-5,6-dihydro-4H-pyrrolo[1,2-b]pyrazol-3-yl]-6-methyl-1H-pyrazolo[3,4-b]pyridine;
4-[(6S)-2-(5-Fluoro-2-pyridyl)-6-methyl-5,6-dihydro-4H-pyrrolo[1,2-b]pyrazol-3-yl]-1H-pyrazolo[3,4-b]pyridine;
4-[(6R)-2-(5-Fluoro-2-pyridyl)-6-methyl-5,6-dihydro-4H-pyrrolo[1,2-b]pyrazol-3-yl]-1H-pyrazolo[3,4-b]pyridine;
4-[(6R)-2-(5-Fluoro-2-pyridyl)-6-methyl-5,6-dihydro-4H-pyrrolo[1,2-b]pyrazol-3-yl]-6-methyl-1H-pyrazolo[3,4-b]pyridine;
8-[(6S)-2-(5-Fluoro-2-pyridyl)-6-methyl-5, 6-dihydro-4H-pyrrolo[1,2-b]pyrazol-3-yl]-2-methoxy-1,5-naphthyridine;
(Racemic) 2-(5-Fluoropyridin-2-yl)-3-(1H-pyrazolo[3,4-b]pyridin-4-yl)-3b,4,4a,5-tetrahydrocyclopropa[3,4]pyrrolo[1,2-b]pyrazole;
(Racemic) 2-(5-Fluoropyridin-2-yl)-3-(6-methyl-1H-pyrazolo[3,4-b]pyridin-4-yl)-3b,4,4a,5-tetrahydrocyclopropa[3,4]pyrrolo[1,2-b]pyrazole;
(4aR,5aR)-2-(5-Fluoropyridin-2-yl)-3-(1H-pyrazolo[3,4-b]pyridin-4-yl)-4,4a,5,5a-tetrahydrocyclopropa[4,5]pyrrolo[1,2-b]pyrazole;
(4aS,5aS)-2-(5-Fluoropyridin-2-yl)-3-(1H-pyrazolo[3,4-b]pyridin-4-yl)-4,4a,5,5a-tetrahydrocyclopropa[4,5]pyrrolo[1,2-b]pyrazole;
(4aR,5aR)-2-(5-Fluoropyridin-2-yl)-3-(6-methyl-1H-pyrazolo[3,4-b]pyridin-4-yl)-4,4a,5,5a-tetrahydrocyclopropa[4,5]pyrrolo[1,2-b]pyrazole;
(4aS,5aS)-2-(5-Fluoropyridin-2-yl)-3-(6-methyl-1H-pyrazolo[3,4-b]pyridin-4-yl)-4,4a,5,5a-tetrahydrocyclopropa[4,5]pyrrolo[1,2-b]pyrazole;
2-(3-(1H-Pyrazolo[3,4-b]pyridin-4-yl)-4,5,6,7-tetrahydropyrazolo[1,5-a]pyridin-2-yl)thiazole;
2-(3-(6-Methyl-1H-pyrazolo[3,4-b]pyridin-4-yl)-4,5,6,7-tetrahydropyrazolo[1,5-a]pyridin-2-yl)thiazole;
4-(3-(1H-Pyrazolo[3,4-b]pyridin-4-yl)-4,5,6,7-tetrahydropyrazolo[1,5-a]pyridin-2-yl)thiazole;
4-(3-(6-Methyl-1H-pyrazolo[3,4-b]pyridin-4-yl)-4,5,6,7-tetrahydropyrazolo[1,5-a]pyridin-2-yl)thiazole;
4-[2-(4-Fluorophenyl)-4,5,6,7-tetrahydropyrazolo[1,5-a]pyridin-3-yl]-1H-pyrrolo[2,3-b]pyridine;
5-Fluoro-4-[2-(5-fluoro-2-pyridyl)-4,5,6,7-tetrahydropyrazolo[1,5-a]pyridin-3-yl]-1H-pyrrolo[2,3-b]pyridine;
4-[2-(5-Fluoro-2-pyridyl)-4,5,6,7-tetrahydropyrazolo[1,5-a]pyridin-3-yl]-1H-pyrazolo[3,4-b]pyridine;
4-[2-(5-Fluoro-2-pyridyl)-4,5,6,7-tetrahydropyrazolo[1,5-a]pyridin-3-yl]-6-methyl-1H-pyrazolo[3,4-b]pyridine;
4-[2-(3, 5-Difluoro-2-pyridyl)-4,5,6,7-tetrahydropyrazolo[1,5-a]pyridin-3-yl]-6-methyl-1H-pyrazolo[3,4-b]pyridine;
4-[2-(3, 5-Difluoro-2-pyridyl)-4,5,6,7-tetrahydropyrazolo[1,5-a]pyridin-3-yl]-1H-pyrazolo[3,4-b]pyridine;
(*R)-4-[2-(4-Fluorophenyl)-7-methyl-4,5,6,7-tetrahydropyrazolo[1,5-a]pyridin-3-yl]-1H-pyrazolo[3,4-b]pyridine;
(*S)-4-[2-(4-Fluorophenyl)-7-methyl-4,5,6,7-tetrahydropyrazolo[1,5-a]pyridin-3-yl]-1H-pyrazolo[3,4-b]pyridine;
4-(6,6-Difluoro-2-(4-fluorophenyl)-4,5,6,7-tetrahydropyrazolo[1,5-a]pyridin-3-yl)pyridin-2-amine;
4-(6,6-Difluoro-2-(4-fluorophenyl)-4,5,6,7-tetrahydropyrazolo[1,5-a]pyridin-3-yl)-1H-pyrazolo[3,4-b]pyridine;
4-[6,6-Difluoro-2-(4-fluorophenyl)-5,7-dihydro-4H-pyrazolo[1,5-a]pyridin-3-yl]-6-methyl-1H-pyrazolo[3,4-b]pyridine;
4-(6,6-Difluoro-2-(5-fluoropyridin-2-yl)-4,5,6,7-tetrahydropyrazolo[1,5-a]pyridin-3-yl)-1H-pyrazolo[3,4-b]pyridine;
4-(6,6-Difluoro-2-(5-fluoropyridin-2-yl)-4,5,6,7-tetrahydropyrazolo[1,5-a]pyridin-3-yl)-6-methyl-1H-pyrazolo[3,4-b]pyridine;
4-(5, 5-Difluoro-2-(4-fluorophenyl)-4,5,6,7-tetrahydropyrazolo[1,5-a]pyridin-3-yl)-1H-pyrazolo[3,4-b]pyridine;
4-(5, 5-Difluoro-2-(4-fluorophenyl)-4,5,6,7-tetrahydropyrazolo[1,5-a]pyridin-3-yl)-6-methyl-1H-pyrazolo[3,4-b]pyridine;
4-[5, 5-Difluoro-2-(5-fluoro-2-pyridyl)-6,7-dihydro-4H-pyrazolo[1,5-a]pyridin-3-yl]-1H-pyrazolo[3,4-b]pyridine;
4-[2-(5-Fluoro-2-pyridyl)-6,6-dimethyl-5,7-dihydro-4H-pyrazolo[1,5-a]pyridin-3-yl]-1H-pyrazolo[3,4-b]pyridine;
4-(2-(5-Fluoropyridin-2-yl)-5,5-dimethyl-4,5,6,7-tetrahydropyrazolo[1,5-a]pyridin-3-yl)-1H-pyrazolo[3,4-b]pyridine;
(*S)-4-[6-(Fluoromethyl)-2-(5-fluoro-2-pyridyl)-6-methyl-5,7-dihydro-4H-pyrazolo[1,5-a]pyridin-3-yl]-1H-pyrazolo[3,4-b]pyridine;
(*R)-4-[6-(Fluoromethyl)-2-(5-fluoro-2-pyridyl)-6-methyl-5,7-dihydro-4H-pyrazolo[1,5-a]pyridin-3-yl]-1H-pyrazolo[3,4-b]pyridine;
(*S)-4-(6-Fluoro-2-(5-fluoropyridin-2-yl)-6-(methoxymethyl)-4,5,6,7-tetrahydropyrazolo[1,5-a]pyridin-3-yl)-6-methyl-1H-pyrazolo[3,4-b]pyridine;
(*R)-4-(6-Fluoro-2-(5-fluoropyridin-2-yl)-6-(methoxymethyl)-4,5,6,7-tetrahydropyrazolo[1,5-a]pyridin-3-yl)-6-methyl-1H-pyrazolo[3,4-b]pyridine;
(*S)-4-(2-(5-Fluoropyridin-2-yl)-6-(methoxymethyl)-6-methyl-4,5,6,7-tetrahydropyrazolo[1,5-a]pyridin-3-yl)-6-methyl-1H-pyrazolo[3,4-b]pyridine;
(*R)-4-(2-(5-Fluoropyridin-2-yl)-6-(methoxymethyl)-6-methyl-4, 5,6,7-tetrahydropyrazolo[1,5-a]pyridin-3-yl)-6-methyl-1H-pyrazolo[3,4-b]pyridine;
(Racemic) 2-(5-Fluoropyridin-2-yl)-3-(6-methyl-1H-pyrazolo[3,4-b]pyridin-4-yl)-5,5a,6,6a-tetrahydro-4H-cyclopropa[e]pyrazolo[1,5-a]pyridine;
(Racemic) 6,6-Difluoro-2-(5-fluoropyridin-2-yl)-3-(1H-pyrazolo[3,4-b]pyridin-4-yl)-5,5a,6,6a-tetrahydro-4H-cyclopropa[e]pyrazolo[1,5-a]pyridine;
(Racemic) 6,6-Difluoro-2-(5-fluoropyridin-2-yl)-3-(6-methyl-1H-pyrazolo[3,4-b]pyridin-4-yl)-5,5a,6,6a-tetrahydro-4H-cyclopropa[e]pyrazolo[1,5-a]pyridine;
(4*R,7*S)-2-(4-Fluorophenyl)-3-(1H-pyrazolo[3,4-b]pyridin-4-yl)-4,5,6,7-tetrahydro-4,7-methanopyrazolo[1,5-a]pyridine;
(4*S,7*R)-2-(4-Fluorophenyl)-3-(1H-pyrazolo[3,4-b]pyridin-4-yl)-4,5,6,7-tetrahydro-4,7-methanopyrazolo[1,5-a]pyridine;
(4*R,7*S)-2-(5-Fluoropyridin-2-yl)-3-(1H-pyrazolo[3,4-b]pyridin-4-yl)-4,5,6,7-tetrahydro-4,7-methanopyrazolo[1,5-a]pyridine;
(4*S,7*R)-2-(5-Fluoropyridin-2-yl)-3-(1H-pyrazolo[3,4-b]pyridin-4-yl)-4,5,6,7-tetrahydro-4,7-methanopyrazolo[1,5-a]pyridine;
2-(4-Fluorophenyl)-3-(1H-pyrazolo[3,4-b]pyridin-4-yl)-4,5,6,7-tetrahydro-4,7-ethanopyrazolo[1,5-a]pyridine;
2-(5-Fluoropyridin-2-yl)-3-(1H-pyrazolo[3,4-b]pyridin-4-yl)-4,5,6,7-tetrahydro-4,7-ethanopyrazolo[1,5-a]pyridine;
2-(4-Fluorophenyl)-3-(6-methyl-1H-pyrazolo[3,4-b]pyridin-4-yl)-4,5,6,7-tetrahydro-4,7-ethanopyrazolo[1,5-a]pyridine;
2-(4-Chlorophenyl)-3-(1H-pyrazolo[3,4-b]pyridin-4-yl)-6,7-dihydro-4H-pyrazolo[5,1-c][1,4]oxazine;
2-(4-Chlorophenyl)-3-(6-methyl-1H-pyrazolo[3,4-b]pyridin-4-yl)-6,7-dihydro-4H-pyrazolo[5,1-c][1,4]oxazine;
2-(4-Fluorophenyl)-3-(1H-pyrrolo[2,3-b]pyridin-4-yl)-6,7-dihydro-4H-pyrazolo[5,1-c][1,4]oxazine;
2-(4-Fluorophenyl)-3-(1H-pyrazolo[3,4-b]pyridin-4-yl)-6,7-dihydro-4H-pyrazolo[5,1-c][1,4]oxazine;
2-(4-Fluorophenyl)-3-(6-methyl-1H-pyrazolo[3,4-b]pyridin-4-yl)-6,7-dihydro-4H-pyrazolo[5,1-c][1,4]oxazine;
2-(4-Chloro-3-fluoro-phenyl)-3-(1H-pyrazolo[3,4-b]pyridin-4-yl)-6,7-dihydro-4H-pyrazolo[5,1-c][1,4]oxazine;
2-(4-Chloro-3-fluoro-phenyl)-3-(6-methyl-1H-pyrazolo[3,4-b]pyridin-4-yl)-6,7-dihydro-4H-pyrazolo[5,1-c][1,4]oxazine;
2-(5-Fluoro-2-pyridyl)-3-(4-pyridyl)-6,7-dihydro-4H-pyrazolo[5,1-c][1,4]oxazine;
3-(2-(Difluoromethyl)-1H-pyrrolo[2,3-b]pyridin-4-yl)-2-(5-fluoropyridin-2-yl)-6,7-dihydro-4H-pyrazolo[5,1-c][1,4]oxazine;
2-(5-Fluoro-3-pyridyl)-3-(1H-pyrazolo[3,4-b]pyridin-4-yl)-6,7-dihydro-4H-pyrazolo[5,1-c][1,4]oxazine;
2-(5-Fluoro-3-pyridyl)-3-(6-methyl-1H-pyrazolo[3,4-b]pyridin-4-yl)-6,7-dihydro-4H-pyrazolo[5,1-c][1,4]oxazine;
2-(5-Fluoro-2-pyridyl)-3-(1H-pyrazolo[3,4-b]pyridin-4-yl)-6,7-dihydro-4H-pyrazolo[5,1-c][1,4]oxazine;
2-(5-Fluoro-2-pyridyl)-3-(6-methyl-1H-pyrazolo[3,4-b]pyridin-4-yl)-6,7-dihydro-4H-pyrazolo[5,1-c][1,4]oxazine;
(R)-2-(5-Fluoropyridin-2-yl)-4-methyl-3-(1H-pyrrolo[2,3-b]pyridin-4-yl)-6,7-dihydro-4H-pyrazolo[5,1-c][1,4]oxazine;
(*S)-2-(4-Fluorophenyl)-6-methyl-3-(1H-pyrazolo[3,4-b]pyridin-4-yl)-6,7-dihydro-4H-pyrazolo[5,1-c][1,4]oxazine;
(*R)-2-(4-Fluorophenyl)-6-methyl-3-(1H-pyrazolo[3,4-b]pyridin-4-yl)-6,7-dihydro-4H-pyrazolo[5,1-c][1,4]oxazine;
(*S)-2-(4-Fluorophenyl)-6-methyl-3-(6-methyl-H-pyrazolo[3,4-b]pyridin-4-yl)-6,7-dihydro-4H-pyrazolo[5,1-c][1,4]oxazine;
(*R)-2-(4-Fluorophenyl)-6-methyl-3-(6-methyl-H-pyrazolo[3,4-b]pyridin-4-yl)-6,7-dihydro-4H-pyrazolo[5,1-c][1,4]oxazine;
(*R)-2-(5-Fluoro-2-pyridyl)-6-methyl-3-(1H-pyrazolo[3,4-b]pyridin-4-yl)-6,7-dihydro-4H-pyrazolo[5,1-c][1,4]oxazine;
(*S)-2-(5-Fluoro-2-pyridyl)-6-methyl-3-(1H-pyrazolo[3,4-b]pyridin-4-yl)-6,7-dihydro-4H-pyrazolo[5,1-c][1,4]oxazine;
(*R)-2-(5-Fluoro-2-pyridyl)-6-methyl-3-(6-methyl-H-pyrazolo[3,4-b]pyridin-4-yl)-6,7-dihydro-4H-pyrazolo[5,1-c][1,4]oxazine;
(*S)-2-(5-Fluoro-2-pyridyl)-6-methyl-3-(6-methyl-H-pyrazolo[3,4-b]pyridin-4-yl)-6,7-dihydro-4H-pyrazolo[5,1-c][1,4]oxazine;
(S)-2-(5-Fluoropyridin-2-yl)-6-methyl-3-(6-methyl-1H-pyrazolo[3,4-b]pyridin-4-yl)-6,7-dihydro-4H-pyrazolo[5,1-c][1,4]oxazine;
(4-(2-(4-Fluorophenyl)-7-methyl-6,7-dihydro-4H-pyrazolo[5,1-c][1,4]oxazin-3-yl)pyridin-2-yl)methanol;
(*S)-2-(4-Fluorophenyl)-7-methyl-3-(1H-pyrazolo[3,4-b]pyridin-4-yl)-6,7-dihydro-4H-pyrazolo[5,1-c][1,4]oxazine;
(*R)-2-(4-Fluorophenyl)-7-methyl-3-(1H-pyrazolo[3,4-b]pyridin-4-yl)-6,7-dihydro-4H-pyrazolo[5,1-c][1,4]oxazine;
(*S)-2-(4-Fluorophenyl)-7-methyl-3-(6-methyl-H-pyrazolo[3,4-b]pyridin-4-yl)-6,7-dihydro-4H-pyrazolo[5,1-c][1,4]oxazine;
(*R)-2-(4-Fluorophenyl)-7-methyl-3-(6-methyl-H-pyrazolo[3,4-b]pyridin-4-yl)-6,7-dihydro-4H-pyrazolo[5,1-c][1,4]oxazine;
(*R)-6-Ethyl-2-(5-fluoropyridin-2-yl)-3-(1H-pyrazolo[3,4-b]pyridin-4-yl)-6,7-dihydro-4H-pyrazolo[5,1-c][1,4]oxazine;
(*S)-6-Ethyl-2-(5-fluoropyridin-2-yl)-3-(1H-pyrazolo[3,4-b]pyridin-4-yl)-6,7-dihydro-4H-pyrazolo[5,1-c][1,4]oxazine;
(*R)-2-(5-Fluoropyridin-2-yl)-6-isopropyl-3-(1H-pyrazolo[3,4-b]pyridin-4-yl)-6,7-dihydro-4H-pyrazolo[5,1-c][1,4]oxazine;
(*S)-2-(5-Fluoropyridin-2-yl)-6-isopropyl-3-(1H-pyrazolo[3,4-b]pyridin-4-yl)-6,7-dihydro-4H-pyrazolo[5,1-c][1,4]oxazine;
2-(4-Fluorophenyl)-3-(1H-pyrazolo[3,4-b]pyridin-4-yl)-6-(trifluoromethyl)-6,7-dihydro-4H-pyrazolo[5,1-c][1,4]oxazine;
(S)-2-(4-Fluorophenyl)-3-(1H-pyrazolo[3,4-b]pyridin-4-yl)-6-(trifluoromethyl)-6,7-dihydro-4H-pyrazolo[5,1-c][1,4]oxazine;
(R)-2-(4-Fluorophenyl)-3-(1H-pyrazolo[3,4-b]pyridin-4-yl)-6-(trifluoromethyl)-6,7-dihydro-4H-pyrazolo[5,1-c][1,4]oxazine;
2-(5-Fluoropyridin-2-yl)-3-(1H-pyrazolo[3,4-b]pyridin-4-yl)-6-(trifluoromethyl)-6,7-dihydro-4H-pyrazolo[5,1-c][1,4]oxazine;
6-(Fluoromethyl)-2-(4-fluorophenyl)-3-(1H-pyrazolo[3,4-b]pyridin-5-yl)-6,7-dihydro-4H-pyrazolo[5,1-c][1,4]oxazine;
(*S)-6-(Fluoromethyl)-2-(4-fluorophenyl)-3-(1H-pyrazolo[3,4-b]pyridin-4-yl)-6,7-dihydro-4H-pyrazolo[5,1-c][1,4]oxazine;
(*R)-6-(Fluoromethyl)-2-(4-fluorophenyl)-3-(1H-pyrazolo[3,4-b]pyridin-4-yl)-6,7-dihydro-4H-pyrazolo[5,1-c][1,4]oxazine;
2′-(4-Fluorophenyl)-3′-(1H-pyrazolo[3,4-b]pyridin-4-yl)-4′H,6′H-spiro[cyclopropane-1,7′-pyrazolo[5,1-c][1,4]oxazine];
2′-(4-Fluorophenyl)-3′-(6-methyl-1H-pyrazolo[3,4-b]pyridin-4-yl)-4′H,6′H-spiro[cyclopropane-1,7′-pyrazolo[5,1-c][1,4]oxazine];
(*R)-6-Cyclopropyl-2-(5-fluoropyridin-2-yl)-3-(1H-pyrazolo[3,4-b]pyridin-4-yl)-6,7-dihydro-4H-pyrazolo[5,1-c][1,4]oxazine;
(*S)-6-Cyclopropyl-2-(5-fluoropyridin-2-yl)-3-(1H-pyrazolo[3,4-b]pyridin-4-yl)-6,7-dihydro-4H-pyrazolo[5,1-c][1,4]oxazine;
(*R)-6-Cyclobutyl-2-(5-fluoropyridin-2-yl)-3-(1H-pyrazolo[3,4-b]pyridin-4-yl)-6,7-dihydro-4H-pyrazolo[5,1-c][1,4]oxazine;
(*S)-6-Cyclobutyl-2-(5-fluoropyridin-2-yl)-3-(1H-pyrazolo[3,4-b]pyridin-4-yl)-6,7-dihydro-4H-pyrazolo[5,1-c][1,4]oxazine;
(6*R,7*S)-2-(4-Fluorophenyl)-6,7-dimethyl-3-(1H-pyrazolo[3,4-b]pyridin-4-yl)-6,7-dihydro-4H-pyrazolo[5,1-c][1,4]oxazine;
(6*S,7*R)-2-(4-Fluorophenyl)-6,7-dimethyl-3-(1H-pyrazolo[3,4-b]pyridin-4-yl)-6,7-dihydro-4H-pyrazolo[5,1-c][1,4]oxazine;
(6*R,7*R)-2-(4-Fluorophenyl)-6,7-dimethyl-3-(1H-pyrazolo[3,4-b]pyridin-4-yl)-6,7-dihydro-4H-pyrazolo[5,1-c][1,4]oxazine;
(6*S,7*S)-2-(4-Fluorophenyl)-6,7-dimethyl-3-(1H-pyrazolo[3,4-b]pyridin-4-yl)-6,7-dihydro-4H-pyrazolo[5,1-c][1,4]oxazine;
(6*R,7*S)-2-(5-Fluoropyridin-2-yl)-6,7-dimethyl-3-(1H-pyrazolo[3,4-b]pyridin-4-yl)-6,7-dihydro-4H-pyrazolo[5,1-c][1,4]oxazine;
(6*S,7*R)-2-(5-Fluoropyridin-2-yl)-6,7-dimethyl-3-(1H-pyrazolo[3,4-b]pyridin-4-yl)-6,7-dihydro-4H-pyrazolo[5,1-c][1,4]oxazine;
(6*R,7*R)-2-(5-Fluoropyridin-2-yl)-6,7-dimethyl-3-(1H-pyrazolo[3,4-b]pyridin-4-yl)-6,7-dihydro-4H-pyrazolo[5,1-c][1,4]oxazine;
(6*S,7*S)-2-(5-Fluoropyridin-2-yl)-6,7-dimethyl-3-(1H-pyrazolo[3,4-b]pyridin-4-yl)-6,7-dihydro-4H-pyrazolo[5,1-c][1,4]oxazine;
2-(4-Fluorophenyl)-7,7-dimethyl-3-(1H-pyrazolo[3,4-b]pyridin-4-yl)-4,6-dihydropyrazolo[5,1-c][1,4]oxazine;
2-(4-Fluorophenyl)-7,7-dimethyl-3-(6-methyl-1H-pyrazolo[3,4-b]pyridin-4-yl)-4,6-dihydropyrazolo[5,1-c][1,4]oxazine;
2-(5-Fluoro-2-pyridyl)-7,7-dimethyl-3-(1H-pyrazolo[3,4-b]pyridin-4-yl)-4,6-dihydropyrazolo[5,1-c][1,4]oxazine;
2-(5-Fluoro-2-pyridyl)-7,7-dimethyl-3-(6-methyl-1H-pyrazolo[3,4-b]pyridin-4-yl)-4,6-dihydropyrazolo[5,1-c][1,4]oxazine;
6,6-Dimethyl-3-(1H-pyrazolo[3,4-b]pyridin-4-yl)-2-(thiazol-4-yl)-6,7-dihydro-4H-pyrazolo[5,1-c][1,4]oxazine;
6,6-Dimethyl-3-(6-methyl-1H-pyrazolo[3,4-b]pyridin-4-yl)-2-(thiazol-4-yl)-6,7-dihydro-4H-pyrazolo[5,1-c][1,4]oxazine;
6,6-Dimethyl-2-(oxazol-5-yl)-3-(1H-pyrazolo[3,4-b]pyridin-4-yl)-6,7-dihydro-4H-pyrazolo[5,1-c][1,4]oxazine;
6,6-Dimethyl-2-(1-methyl-1H-imidazol-4-yl)-3-(1H-pyrazolo[3,4-b]pyridin-4-yl)-6,7-dihydro-4H-pyrazolo[5,1-c][1,4]oxazine;
2-(5-Fluoropyridin-2-yl)-6,6-dimethyl-3-(pyridin-4-yl)-6,7-dihydro-4H-pyrazolo[5,1-c][1,4]oxazin;
3-(2-(Difluoromethyl)pyridin-4-yl)-2-(5-fluoropyridin-2-yl)-6,6-dimethyl-6,7-dihydro-4H-pyrazolo[5,1-c][1,4]oxazine;
2-(5-Fluoropyridin-2-yl)-3-(3-methoxypyridin-4-yl)-6,6-dimethyl-6,7-dihydro-4H-pyrazolo[5,1-c][1,4]oxazine;
N-(4-(2-(5-Fluoropyridin-2-yl)-6,6-dimethyl-6,7-dihydro-4H-pyrazolo[5,1-c][1,4]oxazin-3-yl)pyridin-2-yl)propionamide;
N-(4-(2-(5-Fluoropyridin-2-yl)-6,6-dimethyl-6,7-dihydro-4H-pyrazolo[5,1-c][1,4]oxazin-3-yl)pyridin-2-yl)isobutyramide;
3,3,3-Trifluoro-N-(4-(2-(5-fluoropyridin-2-yl)-6,6-dimethyl-6,7-dihydro-4H-pyrazolo[5,1-c][1,4]oxazin-3-yl)pyridin-2-yl)propenamide;
N-(4-(2-(5-Fluoropyridin-2-yl)-6,6-dimethyl-6,7-dihydro-4H-pyrazolo[5,1-c][1,4]oxazin-3-yl)pyridin-2-yl)cyclopropanecarboxamide;
2,2-Difluoro-N-(4-(2-(5-fluoropyridin-2-yl)-6,6-dimethyl-6,7-dihydro-4H-pyrazolo[5,1-c][1,4]oxazin-3-yl)pyridin-2-yl)cyclopropane-1-carboxamide;
N-(4-(2-(5-Fluoropyridin-2-yl)-6,6-dimethyl-6,7-dihydro-4H-pyrazolo[5,1-c][1,4]oxazin-3-yl)pyridin-2-yl)benzamide;
2-(5-Fluoropyridin-2-yl)-6,6-dimethyl-3-(thieno[3,2-b]pyridin-7-yl)-6,7-dihydro-4H-pyrazolo[5,1-c][1,4]oxazine;
2-(5-Fluoropyridin-2-yl)-6,6-dimethyl-3-(2-methyl-2H-pyrazolo[4,3-b]pyridin-7-yl)-6,7-dihydro-4H-pyrazolo[5,1-c][1,4]oxazine;
2-(5-Fluoropyridin-2-yl)-6,6-dimethyl-3-(1H-pyrazolo[4,3-b]pyridin-7-yl)-6,7-dihydro-4H-pyrazolo[5,1-c][1,4]oxazine;
2-(5-Fluoropyridin-2-yl)-6,6-dimethyl-3-(pyrazolo[1,5-a]pyridin-5-yl)-6,7-dihydro-4H-pyrazolo[5,1-c][1,4]oxazine;
3-(1-Ethyl-1H-pyrazolo[3,4-b]pyridin-5-yl)-2-(5-fluoropyridin-2-yl)-6,6-dimethyl-6,7-dihydro-4H-pyrazolo[5,1-c][1,4]oxazine;
2-(5-Fluoropyridin-2-yl)-6,6-bis(methyl-d₃)-3-(1H-pyrazolo[3,4-b]pyridin-4-yl)-6,7-dihydro-4H-pyrazolo[5,1-c][1,4]oxazine;
2-(5-Fluoropyridin-2-yl)-6,6-dimethyl-3-(1H-pyrazolo[3,4-b]pyridin-4-yl)-6,7-dihydro-4H-pyrazolo[5,1-c][1,4]oxazine-7,7-d₂;
2-(4-Fluorophenyl)-6,6-dimethyl-3-(1H-pyrazolo[3,4-b]pyridin-4-yl)-6,7-dihydro-4H-pyrazolo[5,1-c][1,4]oxazine;
2-(4-Fluorophenyl)-6,6-dimethyl-3-(6-methyl-1H-pyrazolo[3,4-b]pyridin-4-yl)-6,7-dihydro-4H-pyrazolo[5,1-c][1,4]oxazine;
2-(5-Chloropyridin-2-yl)-6,6-dimethyl-3-(1H-pyrazolo[3,4-b]pyridin-4-yl)-6,7-dihydro-4H-pyrazolo[5,1-c][1,4]oxazine;
2-(5-Fluoropyridin-2-yl)-6,6-dimethyl-3-(1H-pyrrolo[2,3-b]pyridin-4-yl)-6,7-dihydro-4H-pyrazolo[5,1-c][1,4]oxazine;
4-(2-(5-Fluoropyridin-2-yl)-6,6-dimethyl-6,7-dihydro-4H-pyrazolo[5,1-c][1,4]oxazin-3-yl)-1H-pyrrolo[2,3-b]pyridine-3-carbonitrile;
2-(5-Fluoro-2-pyridyl)-6,6-dimethyl-3-(1H-pyrrolo[3,2-b]pyridin-7-yl)-4,7-dihydropyrazolo[5,1-c][1,4]oxazine;
2-(5-Fluoropyridin-2-yl)-6,6-dimethyl-3-(1H-pyrazolo[3,4-b]pyridin-4-yl)-6,7-dihydro-4H-pyrazolo[5,1-c][1,4]oxazine;
2-(5-Fluoropyridin-2-yl)-6,6-dimethyl-3-(1H-pyrazolo[3,4-b]pyridin-4-yl)-6,7-dihydro-4H-pyrazolo[5,1-c][1,4]oxazine-4,4-d₂;
3-(3-Bromo-1H-pyrazolo[3,4-b]pyridin-4-yl)-2-(5-fluoropyridin-2-yl)-6,6-dimethyl-6,7-dihydro-4H-pyrazolo[5,1-c][1,4]oxazine;
2-(5-Fluoro-2-pyridyl)-6,6-dimethyl-3-(1-methylpyrazolo[3,4-b]pyridin-4-yl)-4,7-dihydropyrazolo[5,1-c][1,4]oxazine;
2-(5-Fluoro-2-pyridyl)-6,6-dimethyl-3-(6-methyl-1H-pyrazolo[3,4-b]pyridin-4-yl)-4,7-dihydropyrazolo[5,1-c][1,4]oxazine;
3-(6-Cyclopropyl-1H-pyrazolo[3,4-b]pyridin-4-yl)-2-(5-fluoropyridin-2-yl)-6,6-dimethyl-6,7-dihydro-4H-pyrazolo[5,1-c][1,4]oxazine;
3-(3,6-Dimethyl-1H-pyrazolo[3,4-b]pyridin-4-yl)-2-(5-fluoro-2-pyridyl)-6,6-dimethyl-4,7-dihydropyrazolo[5,1-c][1,4]oxazine;
3-(5-Fluoro-6-methyl-1H-pyrazolo[3,4-b]pyridin-4-yl)-2-(5-fluoro-2-pyridyl)-6,6-dimethyl-4,7-dihydropyrazolo[5,1-c][1,4]oxazine;
2-(5-Fluoro-2-pyridyl)-6,6-dimethyl-3-(2-methylpyrazolo[3,4-b]pyridin-4-yl)-4,7-dihydropyrazolo[5,1-c][1,4]oxazine;
2-(5-Fluoropyridin-2-yl)-6,6-dimethyl-3-(1-methyl-1H-pyrazolo[4,3-b]pyridin-7-yl)-6,7-dihydro-4H-pyrazolo[5,1-c][1,4]oxazine;
2-(5-Fluoropyridin-2-yl)-6,6-dimethyl-3-(3-methyl-1H-pyrazolo[3,4-b]pyridin-4-yl)-6,7-dihydro-4H-pyrazolo[5,1-c][1,4]oxazine;
2-(6-Methoxypyridin-2-yl)-6,6-dimethyl-3-(1H-pyrazolo[3,4-b]pyridin-4-yl)-6,7-dihydro-4H-pyrazolo[5,1-c][1,4]oxazine;
2-(3-Chloropyridin-4-yl)-6,6-dimethyl-3-(1H-pyrazolo[3,4-b]pyridin-4-yl)-6,7-dihydro-4H-pyrazolo[5,1-c][1,4]oxazine;
2-(5-Chloro-6-methylpyridin-2-yl)-6,6-dimethyl-3-(1H-pyrazolo[3,4-b]pyridin-4-yl)-6,7-dihydro-4H-pyrazolo[5,1-c][1,4]oxazine;
2-(5-Fluoro-6-methylpyridin-2-yl)-6,6-dimethyl-3-(1H-pyrazolo[3,4-b]pyridin-4-yl)-6,7-dihydro-4H-pyrazolo[5,1-c][1,4]oxazine;
2-(3,5-Difluoropyridin-4-yl)-6,6-dimethyl-3-(1H-pyrazolo[3,4-b]pyridin-4-yl)-6,7-dihydro-4H-pyrazolo[5,1-c][1,4]oxazine;
2-(3,5-Difluoropyridin-2-yl)-6,6-dimethyl-3-(1H-pyrazolo[3,4-b]pyridin-4-yl)-6,7-dihydro-4H-pyrazolo[5,1-c][1,4]oxazine;
2-(3,5-Difluoropyridin-2-yl)-6,6-dimethyl-3-(6-methyl-1H-pyrazolo[3,4-b]pyridin-4-yl)-6,7-dihydro-4H-pyrazolo[5,1-c][1,4]oxazine;
2-(5-Fluoropyridin-2-yl)-3-(6-methoxy-1,5-naphthyridin-4-yl)-6,6-dimethyl-6,7-dihydro-4H-pyrazolo[5,1-c][1,4]oxazine;
(*R)-6-Ethyl-2-(5-fluoropyridin-2-yl)-6-methyl-3-(1H-pyrazolo[3,4-b]pyridin-4-yl)-6,7-dihydro-4H-pyrazolo[5,1-c][1,4]oxazine;
(*S)-6-Ethyl-2-(5-fluoropyridin-2-yl)-6-methyl-3-(1H-pyrazolo[3,4-b]pyridin-4-yl)-6,7-dihydro-4H-pyrazolo[5,1-c][1,4]oxazine;
(*S)-2-(4-Fluorophenyl)-6,6,7-trimethyl-3-(1H-pyrazolo[3,4-b]pyridin-4-yl)-6,7-dihydro-5H-pyrazolo[5,1-b][1,3]oxazine;
(*R)-2-(4-Fluorophenyl)-6,6,7-trimethyl-3-(1H-pyrazolo[3,4-b]pyridin-4-yl)-6,7-dihydro-5H-pyrazolo[5,1-b][1,3]oxazine;
(*R)-2-(5-Fluoropyridin-2-yl)-6,6,7-trimethyl-3-(1H-pyrazolo[3,4-b]pyridin-4-yl)-6,7-dihydro-4H-pyrazolo[5,1-c][1,4]oxazine;
(*S)-2-(5-Fluoropyridin-2-yl)-6,6,7-trimethyl-3-(1H-pyrazolo[3,4-b]pyridin-4-yl)-6,7-dihydro-4H-pyrazolo[5,1-c][1,4]oxazine;
4-(2-(5-Fluoropyridin-2-yl)-6-methyl-6-(trifluoromethyl)-6,7-dihydro-4H-pyrazolo[5,1-c][1,4]oxazin-3-yl)-N-methylpicolinamide;
2-(5-Fluoropyridin-2-yl)-6-methyl-3-(1H-pyrazolo[3,4-b]pyridin-4-yl)-6-(trifluoromethyl)-6,7-dihydro-4H-pyrazolo[5,1-c][1,4]oxazine;
(*S)-2-(5-Fluoropyridin-2-yl)-6-methyl-3-(1H-pyrazolo[3,4-b]pyridin-4-yl)-6-(trifluoromethyl)-6,7-dihydro-4H-pyrazolo[5,1-c][1,4]oxazine;
(*R)-2-(5-Fluoropyridin-2-yl)-6-methyl-3-(1H-pyrazolo[3,4-b]pyridin-4-yl)-6-(trifluoromethyl)-6,7-dihydro-4H-pyrazolo[5,1-c][1,4]oxazine;
(*S)-2-(5-Fluoropyridin-2-yl)-6-methyl-3-(6-methyl-H-pyrazolo[3,4-b]pyridin-4-yl)-6-(trifluoromethyl)-6,7-dihydro-4H-pyrazolo[5,1-c][1,4]oxazine;
(*R)-2-(5-Fluoropyridin-2-yl)-6-methyl-3-(6-methyl-1H-pyrazolo[3,4-b]pyridin-4-yl)-6-(trifluoromethyl)-6,7-dihydro-4H-pyrazolo[5,1-c][1,4]oxazine;
2-(4-Fluorophenyl)-3-(1H-pyrazolo[3,4-b]pyridin-4-yl)-6,7-dihydropyrazolo[5,1-c][1,4]oxazin-4-one;
2-(5-Fluoro-2-pyridyl)-3-(1H-pyrazolo[3,4-b]pyridin-4-yl)-6,7-dihydropyrazolo[5,1-c][1,4]oxazin-4-one;
N-(4-(2-(5-Fluoropyridin-2-yl)-6,7-dihydro-5H-pyrazolo[5,1-b][1,3]oxazin-3-yl)pyridin-2-yl)acetamide;
2-(4-Fluorophenyl)-3-(1H-pyrazolo[3,4-b]pyridin-4-yl)-6,7-dihydro-5H-pyrazolo[5,1-b][1,3]oxazine;
2-(4-Fluorophenyl)-3-(6-methyl-1H-pyrazolo[3,4-b]pyridin-4-yl)-6,7-dihydro-5H-pyrazolo[5,1-b][1,3]oxazine;
2-(5-Fluoropyridin-2-yl)-3-(1H-pyrazolo[3,4-b]pyridin-4-yl)-6,7-dihydro-5H-pyrazolo[5,1-b][1,3]oxazine;
2-(5-Fluoropyridin-2-yl)-3-(6-methyl-1H-pyrazolo[3,4-b]pyridin-4-yl)-6,7-dihydro-5H-pyrazolo[5,1-b][1,3]oxazine;
2-(3,5-Difluoro-2-pyridyl)-3-(1H-pyrazolo[3,4-b]pyridin-4-yl)-6,7-dihydro-5H-pyrazolo[5,1-b][1,3]oxazine;
2-(3,5-Difluoro-2-pyridyl)-3-(6-methyl-1H-pyrazolo[3,4-b]pyridin-4-yl)-6,7-dihydro-5H-pyrazolo[5,1-b][1,3]oxazine;
2-(5-Fluoro-2-pyridyl)-3-(6-methoxy-1,5-naphthyridin-4-yl)-6,7-dihydro-5H-pyrazolo[5,1-b][1,3]oxazine;
(R/S)-2-(4-Fluorophenyl)-6-methyl-3-(1H-pyrazolo[3,4-b]pyridin-4-yl)-6,7-dihydro-5H-pyrazolo[5,1-b][1,3]oxazine;
(R/S)-2-(4-Fluorophenyl)-6-methyl-3-(6-methyl-1H-pyrazolo[3,4-b]pyridin-4-yl)-6,7-dihydro-5H-pyrazolo[5,1-b][1,3]oxazine;
(*S)-2-(4-Fluorophenyl)-6-methyl-3-(6-methyl-H-pyrazolo[3,4-b]pyridin-4-yl)-6,7-dihydro-5H-pyrazolo[5,1-b][1,3]oxazine;
(*R)-2-(4-Fluorophenyl)-6-methyl-3-(6-methyl-H-pyrazolo[3,4-b]pyridin-4-yl)-6,7-dihydro-5H-pyrazolo[5,1-b][1,3]oxazine;
2-(5-Fluoropyridin-2-yl)-6-methyl-3-(thieno[3,2-b]pyridin-7-yl)-6,7-dihydro-5H-pyrazolo[5,1-b][1,3]oxazine;
2-(5-Fluoropyridin-2-yl)-6-methyl-3-(1H-pyrazolo[3,4-b]pyridin-4-yl)-6,7-dihydro-5H-pyrazolo[5,1-b][1,3]oxazine;
2-(5-Fluoropyridin-2-yl)-6-methyl-3-(6-methyl-1H-pyrazolo[3,4-b]pyridin-4-yl)-6,7-dihydro-5H-pyrazolo[5,1-b][1,3]oxazine;
(*S)-2-(5-Fluoro-2-pyridyl)-5-methyl-3-(1H-pyrazolo[3,4-b]pyridin-4-yl)-6,7-dihydro-5H-pyrazolo[5,1-b][1,3]oxazine;
(*R)-2-(5-Fluoro-2-pyridyl)-5-methyl-3-(1H-pyrazolo[3,4-b]pyridin-4-yl)-6,7-dihydro-5H-pyrazolo[5,1-b][1,3]oxazine;
(*S)-2-(5-Fluoro-2-pyridyl)-5-methyl-3-(6-methyl-H-pyrazolo[3,4-b]pyridin-4-yl)-6,7-dihydro-5H-pyrazolo[5,1-b][1,3]oxazine;
(*R)-2-(5-Fluoro-2-pyridyl)-5-methyl-3-(6-methyl-H-pyrazolo[3,4-b]pyridin-4-yl)-6,7-dihydro-5H-pyrazolo[5,1-b][1,3]oxazine;
(5*S,7*R)-2-(5-Fluoro-2-pyridyl)-5,7-dimethyl-3-(1H-pyrazolo[3,4-b]pyridin-4-yl)-6,7-dihydro-5H-pyrazolo[5,1-b][1,3]oxazine;
(5*R,7*S)-2-(5-Fluoro-2-pyridyl)-5,7-dimethyl-3-(1H-pyrazolo[3,4-b]pyridin-4-yl)-6,7-dihydro-5H-pyrazolo[5,1-b][1,3]oxazine;
(5*R,7*R)-2-(5-Fluoropyridin-2-yl)-5,7-dimethyl-3-(1H-pyrazolo[3,4-b]pyridin-4-yl)-6,7-dihydro-5H-pyrazolo[5,1-b][1,3]oxazine;
(5*S,7*S)-2-(5-Fluoropyridin-2-yl)-5,7-dimethyl-3-(1H-pyrazolo[3,4-b]pyridin-4-yl)-6,7-dihydro-5H-pyrazolo[5,1-b][1,3]oxazine;
(5*S,7*R)-2-(5-Fluoro-2-pyridyl)-5,7-dimethyl-3-(6-methyl-1H-pyrazolo[3,4-b]pyridin-4-yl)-6,7-dihydro-5H-pyrazolo[5,1-b][1,3]oxazine;
(5*R,7*S)-2-(5-Fluoro-2-pyridyl)-5,7-dimethyl-3-(6-methyl-1H-pyrazolo[3,4-b]pyridin-4-yl)-6,7-dihydro-5H-pyrazolo[5,1-b][1,3]oxazine;
(5*R,7*R)-2-(5-Fluoro-2-pyridyl)-5,7-dimethyl-3-(6-methyl-1H-pyrazolo[3,4-b]pyridin-4-yl)-6,7-dihydro-5H-pyrazolo[5,1-b][1,3]oxazine;
(5*S,7*S)-2-(5-Fluoro-2-pyridyl)-5,7-dimethyl-3-(6-methyl-1H-pyrazolo[3,4-b]pyridin-4-yl)-6,7-dihydro-5H-pyrazolo[5,1-b][1,3]oxazine;
2-(4-Fluorophenyl)-3-(1H-pyrazolo[3,4-b]pyridin-4-yl)spiro[5,7-dihydropyrazolo[5,1-b][1,3]oxazine-6,3′-oxetane];
2′-(4-Fluorophenyl)-3′-(6-methyl-1H-pyrazolo[3,4-b]pyridin-4-yl)-5′H,7′H-spiro[oxetane-3,6′-pyrazolo[5,1-b][1,3]oxazine];
2-(4-Fluorophenyl)-6,6-dimethyl-3-(1H-pyrazolo[3,4-b]pyridin-4-yl)-6,7-dihydro-5H-pyrazolo[5,1-b][1,3]oxazine;
2-(4-Fluorophenyl)-6,6-dimethyl-3-(6-methyl-1H-pyrazolo[3,4-b]pyridin-4-yl)-6,7-dihydro-5H-pyrazolo[5,1-b][1,3]oxazine;
2-(5-Fluoropyridin-2-yl)-6,6-dimethyl-3-(thieno[3,2-b]pyridin-7-yl)-6,7-dihydro-5H-pyrazolo[5,1-b][1,3]oxazine;
N-(4-(2-(4-Fluorophenyl)-5,6,7,8-tetrahydropyrazolo[5,1-b][1,3]oxazepin-3-yl)pyridin-2-yl)acetamide;
2-(4-Fluorophenyl)-3-(1H-pyrazolo[3,4-b]pyridin-4-yl)-5,6,7,8-tetrahydropyrazolo[5,1-b][1,3]oxazepane;
2-(4-Fluorophenyl)-3-(6-methyl-1H-pyrazolo[3,4-b]pyridin-4-yl)-5,6,7,8-tetrahydropyrazolo[5,1-b][1,3]oxazepane;
2-(5-Fluoro-2-pyridyl)-3-(1H-pyrazolo[3,4-b]pyridin-4-yl)-5,6,7,8-tetrahydropyrazolo[5,1-b][1,3]oxazepane;
2-(5-Fluoro-2-pyridyl)-3-(6-methyl-1H-pyrazolo[3,4-b]pyridin-4-yl)-5,6,7,8-tetrahydropyrazolo[5,1-b][1,3]oxazepane;
7-[2-(5-Fluoro-2-pyridyl)-5,6-dihydro-4H-pyrrolo[1,2-b]pyrazol-3-yl]-1H-pyrazolo[4,3-b]pyridine;
5-Fluoro-4-[2-(5-fluoro-2-pyridyl)-5,6-dihydro-4H-pyrrolo[1,2-b]pyrazol-3-yl]-1H-pyrazolo[3,4-b]pyridine;
6-(Difluoromethyl)-4-[2-(5-fluoro-2-pyridyl)-5,6-dihydro-4H-pyrrolo[1,2-b]pyrazol-3-yl]-1H-pyrazolo[3,4-b]pyridine;
1-Ethyl-5-(2-(5-fluoropyridin-2-yl)-5,6-dihydro-4H-pyrrolo[1,2-b]pyrazol-3-yl)-1H-pyrazolo[3,4-b]pyridine;
3-Chloro-5-fluoro-4-(2-(5-fluoropyridin-2-yl)-5,6-dihydro-4H-pyrrolo[1,2-b]pyrazol-3-yl)-6-methyl-1H-pyrazolo[3,4-b]pyridine;
4-[2-(4-Fluorophenyl)-5,6-dihydro-4H-pyrrolo[1,2-b]pyrazol-3-yl]-6-methyl-1H-pyrazolo[3,4-d]pyrimidine;
5-[(5S)-5-Fluoro-2-(4-fluorophenyl)-5,6-dihydro-4H-pyrrolo[1,2-b]pyrazol-3-yl]pyrazolo[1,5-a]pyridine;
5-Fluoro-4-[(5S)-5-fluoro-2-(4-fluorophenyl)-5,6-dihydro-4H-pyrrolo[1,2-b]pyrazol-3-yl]-1H-pyrazolo[3,4-b]pyridine;
4-[(5R)-5-Fluoro-2-(4-fluorophenyl)-5,6-dihydro-4H-pyrrolo[1,2-b]pyrazol-3-yl]-1H-pyrazolo[3,4-b]pyridine;
N-[4-[(5S)-5-Fluoro-2-(4-fluorophenyl)-5,6-dihydro-4H-pyrrolo[1,2-b]pyrazol-3-yl]-2-pyridyl]acetamide;
4-[5,5-Difluoro-2-(4-fluorophenyl)-4,6-dihydropyrrolo[1,2-b]pyrazol-3-yl]-1H-pyrazolo[3,4-b]pyridine;
4-[2-(4-Fluorophenyl)-5,5-dimethyl-4,6-dihydropyrrolo[1,2-b]pyrazol-3-yl]-1H-pyrazolo[3,4-b]pyridine;
4-[2-(4-Fluorophenyl)-5,5-dimethyl-4,6-dihydropyrrolo[1,2-b]pyrazol-3-yl]-6-methyl-1H-pyrazolo[3,4-b]pyridine;
4-[2-(5-Fluoro-2-pyridyl)-5,5-dimethyl-4,6-dihydropyrrolo[1,2-b]pyrazol-3-yl]-1H-pyrrolo[2,3-b]pyridine;
4-[2-(5-Fluoro-2-pyridyl)-5,5-dimethyl-4,6-dihydropyrrolo[1,2-b]pyrazol-3-yl]-1H-pyrazolo[3,4-b]pyridine;
4-[2-(5-Fluoro-2-pyridyl)-5,5-dimethyl-4,6-dihydropyrrolo[1,2-b]pyrazol-3-yl]-6-methyl-1H-pyrrolo[2,3-b]pyridine;
5-Fluoro-4-[2-(5-fluoro-2-pyridyl)-5,5-dimethyl-4,6-dihydropyrrolo[1,2-b]pyrazol-3-yl]-1H-pyrazolo[3,4-b]pyridine;
3-Chloro-4-(2-(5-fluoropyridin-2-yl)-5,5-dimethyl-5,6-dihydro-4H-pyrrolo[1,2-b]pyrazol-3-yl)-1H-pyrazolo[3,4-b]pyridine;
4-[2-(5-Fluoro-2-pyridyl)-5,5-dimethyl-4,6-dihydropyrrolo[1,2-b]pyrazol-3-yl]-6-methyl-1H-pyrazolo[3,4-b]pyridine;
6-(Difluoromethyl)-4-[2-(5-fluoro-2-pyridyl)-5,5-dimethyl-4,6-dihydropyrrolo[1,2-b]pyrazol-3-yl]-1H-pyrazolo[3,4-b]pyridine;
5-Fluoro-4-[2-(5-fluoro-2-pyridyl)-5,5-dimethyl-4,6-dihydropyrrolo[1,2-b]pyrazol-3-yl]-6-methyl-1H-pyrazolo[3,4-b]pyridine;
4-[2-(4-Fluorophenyl)-5,5-bis(methyl-d₃)-4,6-dihydropyrrolo[1,2-b]pyrazol-3-yl]-1H-pyrazolo[3,4-b]pyridine;
4-[2-(4-Fluorophenyl)-5,5-bis(methyl-d₃)-4,6-dihydropyrrolo[1,2-b]pyrazol-3-yl]-6-methyl-1H-pyrazolo[3,4-b]pyridine;
5-Fluoro-4-[2-(5-fluoro-2-pyridyl)-5,5-bis(methyl-d₃)-4,6-dihydropyrrolo[1,2-b]pyrazol-3-yl]-1H-pyrazolo[3,4-b]pyridine;
5-Fluoro-4-[2-(5-fluoro-2-pyridyl)-5,5-bis(methyl-d₃)-4,6-dihydropyrrolo[1,2-b]pyrazol-3-yl]-6-methyl-1H-pyrazolo[3,4-b]pyridine;
4-[2-(4-Fluorophenyl)-4,4-dimethyl-5,6-dihydropyrrolo[1,2-b]pyrazol-3-yl]-1H-pyrazolo[3,4-b]pyridine;
4-[2-(4-Fluorophenyl)-4,4-dimethyl-5,6-dihydropyrrolo[1,2-b]pyrazol-3-yl]-6-methyl-1H-pyrazolo[3,4-b]pyridine;
2-(4-Fluorophenyl)-3-(1H-pyrazolo[3,4-b]pyridin-4-yl)spiro[4,6-dihydropyrrolo[1,2-b]pyrazole-5,1′-cyclopropane];
2-(4-Fluorophenyl)-3-(6-methyl-1H-pyrazolo[3,4-b]pyridin-4-yl)spiro[4,6-dihydropyrrolo[1,2-b]pyrazole-5,1′-cyclopropane];
2-(5-Fluoro-2-pyridyl)-3-(1H-pyrazolo[3,4-b]pyridin-4-yl)spiro[4,5-dihydropyrrolo[1,2-b]pyrazole-6,1′-cyclopropane];
(*S)-1′,1′-Difluoro-2-(4-fluorophenyl)-3-(1H-pyrazolo[3,4-b]pyridin-4-yl)spiro[4,6-dihydropyrrolo[1,2-b]pyrazole-5,2′-cyclopropane];
(*R)-1′,1′-Difluoro-2-(4-fluorophenyl)-3-(1H-pyrazolo[3,4-b]pyridin-4-yl)spiro[4,6-dihydropyrrolo[1,2-b]pyrazole-5,2′-cyclopropane];
(*S)-1′,1′-Difluoro-2-(4-fluorophenyl)-3-(6-methyl-1H-pyrazolo[3,4-b]pyridin-4-yl)spiro[4,6-dihydropyrrolo[1,2-b]pyrazole-5,2′-cyclopropane];
(*R)-1′,1′-Difluoro-2-(4-fluorophenyl)-3-(6-methyl-1H-pyrazolo[3,4-b]pyridin-4-yl)spiro[4,6-dihydropyrrolo[1,2-b]pyrazole-5,2′-cyclopropane];
(*S)-1′,1′-Difluoro-2-(5-fluoro-2-pyridyl)-3-(1H-pyrazolo[3,4-b]pyridin-4-yl)spiro[4,6-dihydropyrrolo[1,2-b]pyrazole-5,2′-cyclopropane];
(*R)-1′,1′-Difluoro-2-(5-fluoro-2-pyridyl)-3-(1H-pyrazolo[3,4-b]pyridin-4-yl)spiro[4,6-dihydropyrrolo[1,2-b]pyrazole-5,2′-cyclopropane];
(*S)-3-(3-Chloro-1H-pyrazolo[3,4-b]pyridin-4-yl)-1′,1′-difluoro-2-(5-fluoro-2-pyridyl)spiro[4,6-dihydropyrrolo[1,2-b]pyrazole-5,2′-cyclopropane];
(*R)-3-(3-Chloro-1H-pyrazolo[3,4-b]pyridin-4-yl)-1′,1′-difluoro-2-(5-fluoro-2-pyridyl)spiro[4,6-dihydropyrrolo[1,2-b]pyrazole-5,2′-cyclopropane];
(*S)-2,2-Difluoro-3′-(5-fluoro-1H-pyrazolo[3,4-b]pyridin-4-yl)-2′-(5-fluoropyridin-2-yl)-4′H,6′H-spiro[cyclopropane-1,5′-pyrrolo[1,2-b]pyrazole];
(*R)-1′,1′-Difluoro-3-(5-fluoro-1H-pyrazolo[3,4-b]pyridin-4-yl)-2-(5-fluoro-2-pyridyl)spiro[4,6-dihydropyrrolo[1,2-b]pyrazole-5,2′-cyclopropane];
(*S)-1′,1′-Difluoro-2-(5-fluoro-2-pyridyl)-3-(6-methyl-1H-pyrazolo[3,4-b]pyridin-4-yl)spiro[4,6-dihydropyrrolo[1,2-b]pyrazole-5,2′-cyclopropane];
(*R)-1′,1′-Difluoro-2-(5-fluoro-2-pyridyl)-3-(6-methyl-1H-pyrazolo[3,4-b]pyridin-4-yl)spiro[4,6-dihydropyrrolo[1,2-b]pyrazole-5,2′-cyclopropane];
(*S)-3′-(3-Chloro-6-methyl-1H-pyrazolo[3,4-b]pyridin-4-yl)-2,2-difluoro-2′-(5-fluoropyridin-2-yl)-4′H,6′H-spiro[cyclopropane-1,5′-pyrrolo[1,2-b]pyrazole];
(*R)-1′,1′-Difluoro-3-(5-fluoro-6-methyl-1H-pyrazolo[3,4-b]pyridin-4-yl)-2-(5-fluoro-2-pyridyl)spiro[4,6-dihydropyrrolo[1,2-b]pyrazole-5,2′-cyclopropane];
(*S)-1′,1′-Difluoro-3-(5-fluoro-6-methyl-1H-pyrazolo[3,4-b]pyridin-4-yl)-2-(5-fluoro-2-pyridyl)spiro[4,6-dihydropyrrolo[1,2-b]pyrazole-5,2′-cyclopropane];
(*R)-1′,1′-Difluoro-3-(5-fluoro-3, 6-dimethyl-1H-pyrazolo[3,4-b]pyridin-4-yl)-2-(5-fluoro-2-pyridyl)spiro[4,6-dihydropyrrolo[1,2-b]pyrazole-5,2′-cyclopropane];
(*S)-1′,1′-Difluoro-3-(5-fluoro-3, 6-dimethyl-1H-pyrazolo[3,4-b]pyridin-4-yl)-2-(5-fluoro-2-pyridyl)spiro[4,6-dihydropyrrolo[1,2-b]pyrazole-5,2′-cyclopropane];
(4aR,5aR)-2-(4-Fluorophenyl)-3-(1H-pyrazolo[3,4-b]pyridin-4-yl)-4,4a,5,5a-tetrahydrocyclopropa[4,5]pyrrolo[1,2-b]pyrazole;
(4aS,5aS)-2-(4-Fluorophenyl)-3-(1H-pyrazolo[3,4-b]pyridin-4-yl)-4,4a,5,5a-tetrahydrocyclopropa[4,5]pyrrolo[1,2-b]pyrazole;
(4aR,5aR)-2-(4-Fluorophenyl)-3-(6-methyl-1-((2-(trimethylsilyl)ethoxy)methyl)-1H-pyrazolo[3,4-b]pyridin-4-yl)-4,4a,5,5a-tetrahydrocyclopropa[4,5]pyrrolo[1,2-b]pyrazole;
(4aS,5aS)-2-(4-Fluorophenyl)-3-(6-methyl-1-((2-(trimethylsilyl)ethoxy)methyl)-1H-pyrazolo[3,4-b]pyridin-4-yl)-4,4a,5,5a-tetrahydrocyclopropa[4,5]pyrrolo[1,2-b]pyrazole;
(4aS,5aS)-3-(3-Chloro-1H-pyrazolo[3,4-b]pyridin-4-yl)-2-(5-fluoropyridin-2-yl)-4,4a,5,5a-tetrahydrocyclopropa[4,5]pyrrolo[1,2-b]pyrazole;
(4aS,5aS)-3-(5-Fluoro-1H-pyrazolo[3,4-b]pyridin-4-yl)-2-(5-fluoropyridin-2-yl)-4,4a,5,5a-tetrahydrocyclopropa[4,5]pyrrolo[1,2-b]pyrazole;
(4aS,5aS)-3-(5-Fluoro-6-methyl-1H-pyrazolo[3,4-b]pyridin-4-yl)-2-(5-fluoropyridin-2-yl)-4,4a,5,5a-tetrahydrocyclopropa[4,5]pyrrolo[1,2-b]pyrazole;
(4aS,5aS)-3-(5-Fluoro-3,6-dimethyl-1H-pyrazolo[3,4-b]pyridin-4-yl)-2-(5-fluoropyridin-2-yl)-4,4a,5,5a-tetrahydrocyclopropa[4,5]pyrrolo[1,2-b]pyrazole;
(Racemic) 2-(4-Fluorophenyl)-3-(1H-pyrazolo[3,4-b]pyridin-4-yl)-3b,4,4a,5-tetrahydrocyclopropa[3,4]pyrrolo[1,2-b]pyrazole;
(3b*R,4a*S)-2-(4-Fluorophenyl)-3-(6-methyl-1H-pyrazolo[3,4-b]pyridin-4-yl)-3b,4,4a,5-tetrahydrocyclopropa[3,4]pyrrolo[1,2-b]pyrazole;
(3b*S,4a*R)-2-(4-Fluorophenyl)-3-(6-methyl-1H-pyrazolo[3,4-b]pyridin-4-yl)-3b,4,4a,5-tetrahydrocyclopropa[3,4]pyrrolo[1,2-b]pyrazole;
4-[5,5-Difluoro-2-(4-fluorophenyl)-6,7-dihydro-4H-pyrazolo[1,5-a]pyridin-3-yl]-5-fluoro-1H-pyrazolo[3,4-b]pyridine;
4-[6,6-Difluoro-2-(4-fluorophenyl)-5,7-dihydro-4H-pyrazolo[1,5-a]pyridin-3-yl]-6-methyl-1H-pyrazolo[3,4-d]pyrimidine;
4-(6,6-Difluoro-2-(4-fluorophenyl)-4,5,6,7-tetrahydropyrazolo[1,5-a]pyridin-3-yl)-5-fluoro-1H-pyrazolo[3,4-b]pyridine;
4-[2-(5-Fluoro-2-pyridyl)-6,6-dimethyl-5,7-dihydro-4H-pyrazolo[1,5-a]pyridin-3-yl]pyridin-2-amine;
5-Fluoro-4-[2-(5-fluoro-2-pyridyl)-6,6-dimethyl-5,7-dihydro-4H-pyrazolo[1,5-a]pyridin-3-yl]-1H-pyrazolo[3,4-b]pyridine;
4-[2-(5-Fluoro-2-pyridyl)-6,6-bis(methyl-d₃)-5,7-dihydro-4H-pyrazolo[1,5-a]pyridin-3-yl]-1H-pyrazolo[3,4-b]pyridine;
(*S)-6-(Fluoromethyl)-2-(5-fluoro-2-pyridyl)-6-methyl-3-(4-pyridyl)-5,7-dihydro-4H-pyrazolo[1,5-a]pyridine;
(*R)-6-(Fluoromethyl)-2-(5-fluoro-2-pyridyl)-6-methyl-3-(4-pyridyl)-5,7-dihydro-4H-pyrazolo[1,5-a]pyridine;
(*S)-4-[6-(Fluoromethyl)-2-(5-fluoro-2-pyridyl)-6-methyl-5,7-dihydro-4H-pyrazolo[1,5-a]pyridin-3-yl]pyridin-2-amine;
(*R)-4-[6-(Fluoromethyl)-2-(5-fluoro-2-pyridyl)-6-methyl-5,7-dihydro-4H-pyrazolo[1,5-a]pyridin-3-yl]pyridin-2-amine;
(Racemic) 3-(2,5-Difluoro-4-pyridyl)-6-(fluoromethyl)-2-(5-fluoro-2-pyridyl)-6-methyl-5,7-dihydro-4H-pyrazolo[1,5-a]pyridine;
(*R)-5-[6-(Fluoromethyl)-2-(5-fluoro-2-pyridyl)-6-methyl-5,7-dihydro-4H-pyrazolo[1,5-a]pyridin-3-yl]pyrazolo[1,5-a]pyridine;
(*S)-5-[6-(Fluoromethyl)-2-(5-fluoro-2-pyridyl)-6-methyl-5,7-dihydro-4H-pyrazolo[1,5-a]pyridin-3-yl]pyrazolo[1,5-a]pyridine;
(*R)-3-Chloro-4-(6-(fluoromethyl)-2-(5-fluoropyridin-2-yl)-6-methyl-4,5,6,7-tetrahydropyrazolo[1,5-a]pyridin-3-yl)-1H-pyrazolo[3,4-b]pyridine;
(*R)-5-Fluoro-4-[6-(fluoromethyl)-2-(5-fluoro-2-pyridyl)-6-methyl-5,7-dihydro-4H-pyrazolo[1,5-a]pyridin-3-yl]-1H-pyrazolo[3,4-b]pyridine;
(*S)-5-Fluoro-4-[6-(fluoromethyl)-2-(5-fluoro-2-pyridyl)-6-methyl-5,7-dihydro-4H-pyrazolo[1,5-a]pyridin-3-yl]-1H-pyrazolo[3,4-b]pyridine;
(*R)-5-Fluoro-4-(6-(fluoromethyl-d₂)-2-(5-fluoropyridin-2-yl)-6-(methyl-d₃)-4,5,6,7-tetrahydropyrazolo[1,5-a]pyridin-3-yl)-1H-pyrazolo[3,4-b]pyridine;
(*S)-5-Fluoro-4-(6-(fluoromethyl-d₂)-2-(5-fluoropyridin-2-yl)-6-(methyl-d)-4,5,6,7-tetrahydropyrazolo[1,5-a]pyridin-3-yl)-1H-pyrazolo[3,4-b]pyridine;
(*S)-4-[2-(5-Fluoro-2-pyridyl)-6-(methoxymethyl)-4,5,6,7-tetrahydropyrazolo[1,5-a]pyridin-3-yl]-6-methyl-1H-pyrazolo[3,4-b]pyridine;
(*R)-4-[2-(5-Fluoro-2-pyridyl)-6-(methoxymethyl)-4,5,6,7-tetrahydropyrazolo[1,5-a]pyridin-3-yl]-6-methyl-1H-pyrazolo[3,4-b]pyridine;
(*S)-4-[6-Fluoro-2-(5-fluoro-2-pyridyl)-6-(methoxymethyl)-5,7-dihydro-4H-pyrazolo[1,5-a]pyridin-3-yl]-6-methyl-1H-pyrrolo[2,3-b]pyridine;
(*R)-4-[6-Fluoro-2-(5-fluoro-2-pyridyl)-6-(methoxymethyl)-5,7-dihydro-4H-pyrazolo[1,5-a]pyridin-3-yl]-6-methyl-1H-pyrrolo[2,3-b]pyridine;
(*S)-4-[6-Fluoro-2-(5-fluoro-2-pyridyl)-6-(methoxymethyl)-5,7-dihydro-4H-pyrazolo[1,5-a]pyridin-3-yl]-1H-pyrazolo[3,4-b]pyridine;
(*R)-4-[6-Fluoro-2-(5-fluoro-2-pyridyl)-6-(methoxymethyl)-5,7-dihydro-4H-pyrazolo[1,5-a]pyridin-3-yl]-1H-pyrazolo[3,4-b]pyridine;
(*S)-5-Fluoro-4-[6-fluoro-2-(5-fluoro-2-pyridyl)-6-(methoxymethyl)-5,7-dihydro-4H-pyrazolo[1,5-a]pyridin-3-yl]-1H-pyrazolo[3,4-b]pyridine;
(*R)-5-Fluoro-4-[6-fluoro-2-(5-fluoro-2-pyridyl)-6-(methoxymethyl)-5,7-dihydro-4H-pyrazolo[1,5-a]pyridin-3-yl]-1H-pyrazolo[3,4-b]pyridine;
(*S)-4-[6-Fluoro-2-(5-fluoro-2-pyridyl)-6-(methoxymethyl)-5,7-dihydro-4H-pyrazolo[1,5-a]pyridin-3-yl]-6-methyl-1H-pyrazolo[3,4-b]pyridine;
(*R)-4-[6-Fluoro-2-(5-fluoro-2-pyridyl)-6-(methoxymethyl)-5,7-dihydro-4H-pyrazolo[1,5-a]pyridin-3-yl]-6-methyl-1H-pyrazolo[3,4-b]pyridine;
(*S)-6-(Difluoromethyl)-4-[6-fluoro-2-(5-fluoro-2-pyridyl)-6-(methoxymethyl)-5,7-dihydro-4H-pyrazolo[1,5-a]pyridin-3-yl]-1H-pyrazolo[3,4-b]pyridine;
(*R)-6-(Difluoromethyl)-4-[6-fluoro-2-(5-fluoro-2-pyridyl)-6-(methoxymethyl)-5,7-dihydro-4H-pyrazolo[1,5-a]pyridin-3-yl]-1H-pyrazolo[3,4-b]pyridine;
(*S)-5-Fluoro-4-[6-fluoro-2-(5-fluoro-2-pyridyl)-6-(methoxymethyl)-5,7-dihydro-4H-pyrazolo[1,5-a]pyridin-3-yl]-6-methyl-1H-pyrazolo[3,4-b]pyridine;
(*R)-5-Fluoro-4-[6-fluoro-2-(5-fluoro-2-pyridyl)-6-(methoxymethyl)-5,7-dihydro-4H-pyrazolo[1,5-a]pyridin-3-yl]-6-methyl-1H-pyrazolo[3,4-b]pyridine;
(*S)-4-(6-Fluoro-2-(5-fluoropyridin-2-yl)-6-((methoxy-d₃)methyl)-4,5,6,7-tetrahydropyrazolo[1,5-a]pyridin-3-yl)-6-methyl-1H-pyrazolo[3,4-b]pyridine;
(*R)-4-(6-Fluoro-2-(5-fluoropyridin-2-yl)-6-((methoxy-d₃)methyl)-4,5,6,7-tetrahydropyrazolo[1,5-a]pyridin-3-yl)-6-methyl-1H-pyrazolo[3,4-b]pyridine;
(*S)-4-[6-[(Methoxy-d₃)methyl-d₂]-6-fluoro-2-(5-fluoro-2-pyridyl)-5,7-dihydro-4H-pyrazolo[1,5-a]pyridin-3-yl]-6-methyl-1H-pyrazolo[3,4-b]pyridine;
(*R)-4-[6-[(Methoxy-d₃)methyl-d₂]-6-fluoro-2-(5-fluoro-2-pyridyl)-5,7-dihydro-4H-pyrazolo[1,5-a]pyridin-3-yl]-6-methyl-1H-pyrazolo[3,4-b]pyridine;
(*S)-4-(6-((Difluoromethoxy)methyl)-6-fluoro-2-(5-fluoropyridin-2-yl)-4,5,6,7-tetrahydropyrazolo[1,5-a]pyridin-3-yl)-1H-pyrazolo[3,4-b]pyridine;
(*R)-4-(6-((Difluoromethoxy)methyl)-6-fluoro-2-(5-fluoropyridin-2-yl)-4,5,6,7-tetrahydropyrazolo[1,5-a]pyridin-3-yl)-1H-pyrazolo[3,4-b]pyridine;
(*S)-4-[6-Fluoro-2-(5-fluoro-2-pyridyl)-6-(trifluoromethoxymethyl)-5,7-dihydro-4H-pyrazolo[1,5-a]pyridin-3-yl]-1H-pyrazolo[3,4-b]pyridine;
(*R)-4-[6-Fluoro-2-(5-fluoro-2-pyridyl)-6-(trifluoromethoxymethyl)-5,7-dihydro-4H-pyrazolo[1,5-a]pyridin-3-yl]-1H-pyrazolo[3,4-b]pyridine;
(*S)-4-[2-(5-Fluoro-2-pyridyl)-6-(2-methoxyethyl)-6-methyl-5,7-dihydro-4H-pyrazolo[1,5-a]pyridin-3-yl]-1H-pyrazolo[3,4-b]pyridine;
(*R)-4-[2-(5-Fluoro-2-pyridyl)-6-(2-methoxyethyl)-6-methyl-5,7-dihydro-4H-pyrazolo[1,5-a]pyridin-3-yl]-1H-pyrazolo[3,4-b]pyridine;
(*R)-4-(2-(5-Fluoropyridin-2-yl)-6-methyl-6-(oxetan-3-ylmethyl)-4,5,6,7-tetrahydropyrazolo[1,5-a]pyridin-3-yl)-1H-pyrazolo[3,4-b]pyridine;
(*S)-4-(2-(5-Fluoropyridin-2-yl)-6-methyl-6-(oxetan-3-ylmethyl)-4,5,6,7-tetrahydropyrazolo[1,5-a]pyridin-3-yl)-1H-pyrazolo[3,4-b]pyridine;
(Racemic) 2-(5-Fluoropyridin-2-yl)-6-(methyl-d₃)-3-(1H-pyrazolo[3,4-b]pyridin-4-yl)-4,5,6,7-tetrahydropyrazolo[1,5-a]pyridine-6-carbonitrile;
(*S)-2-(5-Fluoropyridin-2-yl)-6-(methyl-d₃)-3-(1H-pyrazolo[3,4-b]pyridin-4-yl)-4,5,6,7-tetrahydropyrazolo[1,5-a]pyridine-6-carbonitrile;
(*R)-2-(5-Fluoropyridin-2-yl)-6-(methyl-d₃)-3-(1H-pyrazolo[3,4-b]pyridin-4-yl)-4,5,6,7-tetrahydropyrazolo[1,5-a]pyridine-6-carbonitrile;
(*R)-2,2-Difluoro-2′-(4-fluorophenyl)-3′-(1H-pyrazolo[3,4-b]pyridin-4-yl)-4′,5′-dihydro-7′H-spiro[cyclopropane-1,6′-pyrazolo[1,5-a]pyridine];
(*S)-2,2-Difluoro-2′-(4-fluorophenyl)-3′-(1H-pyrazolo[3,4-b]pyridin-4-yl)-4′,5′-dihydro-7′H-spiro[cyclopropane-1,6′-pyrazolo[1,5-a]pyridine];
(*S)-2,2-Difluoro-2′-(5-fluoropyridin-2-yl)-3′-(1H-pyrazolo[3,4-b]pyridin-4-yl)-4′,5′-dihydro-7′H-spiro[cyclopropane-1,6′-pyrazolo[1,5-a]pyridine];
(*R)-2,2-Difluoro-2′-(5-fluoropyridin-2-yl)-3′-(1H-pyrazolo[3,4-b]pyridin-4-yl)-4′,5′-dihydro-7′H-spiro[cyclopropane-1,6′-pyrazolo[1,5-a]pyridine];
(*S)-2,2-Difluoro-3′-(5-fluoro-1H-pyrazolo[3,4-b]pyridin-4-yl)-2′-(5-fluoropyridin-2-yl)-4′,5′-dihydro-7′H-spiro[cyclopropane-1,6′-pyrazolo[1,5-a]pyridine];
(*R)-2,2-Difluoro-3′-(5-fluoro-1H-pyrazolo[3,4-b]pyridin-4-yl)-2′-(5-fluoropyridin-2-yl)-4′,5′-dihydro-7′H-spiro[cyclopropane-1,6′-pyrazolo[1,5-a]pyridine;
(*S)-2,2-Difluoro-3′-(5-fluoro-6-methyl-1H-pyrazolo[3,4-b]pyridin-4-yl)-2′-(5-fluoropyridin-2-yl)-4′,5′-dihydro-7′H-spiro[cyclopropane-1,6′-pyrazolo[1,5-a]pyridine];
(*R)-2,2-Difluoro-3′-(5-fluoro-6-methyl-1H-pyrazolo[3,4-b]pyridin-4-yl)-2′-(5-fluoropyridin-2-yl)-4′,5′-dihydro-7′H-spiro[cyclopropane-1,6′-pyrazolo[1,5-a]pyridine];
(*S)-2,2-Difluoro-3′-(5-fluoro-3,6-dimethyl-1H-pyrazolo[3,4-b]pyridin-4-yl)-2′-(5-fluoropyridin-2-yl)-4′,5′-dihydro-7′H-spiro[cyclopropane-1,6′-pyrazolo[1,5-a]pyridine];
(*R)-2,2-Difluoro-3′-(5-fluoro-3,6-dimethyl-1H-pyrazolo[3,4-b]pyridin-4-yl)-2′-(5-fluoropyridin-2-yl)-4′,5′-dihydro-7′H-spiro[cyclopropane-1,6′-pyrazolo[1,5-a]pyridine];
(*S)-2,2-Difluoro-2′-(4-fluorophenyl)-3′-(6-methyl-1H-pyrazolo[3,4-d]pyrimidin-4-yl)-4′,5′-dihydro-7′H-spiro[cyclopropane-1,6′-pyrazolo[1,5-a]pyridine];
(*R)-2,2-Difluoro-2′-(4-fluorophenyl)-3′-(6-methyl-H-pyrazolo[3,4-d]pyrimidin-4-yl)-4′,5′-dihydro-7′H-spiro[cyclopropane-1,6′-pyrazolo[1,5-a]pyridine];
(1*S,4′*S)-4′-Chloro-2,2-difluoro-2′-(5-fluoropyridin-2-yl)-3′-(1H-pyrazolo[3,4-b]pyridin-4-yl)-4′,5′-dihydro-7′H-spiro[cyclopropane-1,6′-pyrazolo[1,5-a]pyridine];
(1*S,4′*R)-4′-Chloro-2,2-difluoro-2′-(5-fluoropyridin-2-yl)-3′-(1H-pyrazolo[3,4-b]pyridin-4-yl)-4′,5′-dihydro-7′H-spiro[cyclopropane-1,6′-pyrazolo[1,5-a]pyridine];
(5a*S,6a*R)-2-(4-Fluorophenyl)-3-(1H-pyrazolo[3,4-b]pyridin-4-yl)-5,5a,6,6a-tetrahydro-4H-cyclopropa[e]pyrazolo[1,5-a]pyridine;
(5a*R,6a*S)-2-(4-Fluorophenyl)-3-(1H-pyrazolo[3,4-b]pyridin-4-yl)-5,5a,6,6a-tetrahydro-4H-cyclopropa[e]pyrazolo[1,5-a]pyridine;
N-(4-((5a*R,6a*S)-2-(5-Fluoropyridin-2-yl)-5,5a,6,6a-tetrahydro-4H-cyclopropa[e]pyrazolo[1,5-a]pyridin-3-yl)pyridin-2-yl)acetamide;
N-(4-((5a*S, 6a*R)-2-(5-Fluoropyridin-2-yl)-5, 5a,6,6a-tetrahydro-4H-cyclopropa[e]pyrazolo[1,5-a]pyridin-3-yl)pyridin-2-yl)acetamide;
(5a*R,6a*S)-3-(5-Fluoro-1H-pyrazolo[3,4-b]pyridin-4-yl)-2-(5-fluoropyridin-2-yl)-5,5a,6,6a-tetrahydro-4H-cyclopropa[e]pyrazolo[1,5-a]pyridine;
(5a*R,6a*S)-3-(5-Fluoro-6-methyl-1H-pyrazolo[3,4-b]pyridin-4-yl)-2-(5-fluoropyridin-2-yl)-5,5a,6,6a-tetrahydro-4H-cyclopropa[e]pyrazolo[1,5-a]pyridine;
(5a*R,6a*S)-3-(5-Fluoro-3-methyl-1H-pyrazolo[3,4-b]pyridin-4-yl)-2-(5-fluoropyridin-2-yl)-5,5a,6,6a-tetrahydro-4H-cyclopropa[e]pyrazolo[1,5-a]pyridine;
(5a*R,6a*S)-3-(5-Fluoro-3,6-dimethyl-1H-pyrazolo[3,4-b]pyridin-4-yl)-2-(5-fluoropyridin-2-yl)-5,5a,6,6a-tetrahydro-4H-cyclopropa[e]pyrazolo[1,5-a]pyridine;
(5a*R,6a*S)-2-(5-Fluoropyridin-2-yl)-3-(6-methyl-1H-pyrazolo[3,4-d]pyrimidin-4-yl)-5,5a,6,6a-tetrahydro-4H-cyclopropa[e]pyrazolo[1,5-a]pyridine;
(Racemic) N-(4-(6,6-Difluoro-2-(4-fluorophenyl)-5,5a,6,6a-tetrahydro-4H-cyclopropa[e]pyrazolo[1,5-a]pyridin-3-yl)pyridin-2-yl)acetamide;
N-(4-((5a*R,6a*S)-6,6-Difluoro-2-(4-fluorophenyl)-5, 5a,6,6a-tetrahydro-4H-cyclopropa[e]pyrazolo[1,5-a]pyridin-3-yl)pyridin-2-yl)acetamide;
N-(4-((5a*S, 6a*R)-6, 6-Difluoro-2-(4-fluorophenyl)-5,5a,6,6a-tetrahydro-4H-cyclopropa[e]pyrazolo[1,5-a]pyridin-3-yl)pyridin-2-yl)acetamide;
N-(4-((5a*S,6a*R)-6,6-Difluoro-2-(5-fluoropyridin-2-yl)-5,5a,6,6a-tetrahydro-4H-cyclopropa[e]pyrazolo[1,5-a]pyridin-3-yl)pyridin-2-yl)acetamide;
N-(4-((5a*R,6a*S)-6,6-Difluoro-2-(5-fluoropyridin-2-yl)-5,5a,6,6a-tetrahydro-4H-cyclopropa[e]pyrazolo[1,5-a]pyridin-3-yl)pyridin-2-yl)acetamide;
(5a*S,6a*R)-6, 6-Difluoro-2-(4-fluorophenyl)-3-(pyrazolo[1,5-a]pyridin-4-yl)-5,5a,6,6a-tetrahydro-4H-cyclopropa[e]pyrazolo[1,5-a]pyridine;
(5a*R,6a*S)-6,6-Difluoro-2-(4-fluorophenyl)-3-(pyrazolo[1,5-a]pyridin-4-yl)-5,5a,6,6a-tetrahydro-4H-cyclopropa[e]pyrazolo[1,5-a]pyridine;
(5a*S,6a*R)-6, 6-Difluoro-2-(4-fluorophenyl)-3-(1H-pyrazolo[3,4-b]pyridin-4-yl)-5,5a,6,6a-tetrahydro-4H-cyclopropa[e]pyrazolo[1,5-a]pyridine;
(5a*R, 6a*S)-6, 6-Difluoro-2-(4-fluorophenyl)-3-(1H-pyrazolo[3,4-b]pyridin-4-yl)-5,5a,6,6a-tetrahydro-4H-cyclopropa[e]pyrazolo[1,5-a]pyridine;
(5a*S,6a*R)-6, 6-Difluoro-3-(5-fluoro-6-methyl-1H-pyrazolo[3,4-b]pyridin-4-yl)-2-(5-fluoropyridin-2-yl)-5, 5a,6,6a-tetrahydro-4H-cyclopropa[e]pyrazolo[1,5-a]pyridine;
(5a*S,6a*R)-6, 6-Difluoro-3-(5-fluoro-1H-pyrazolo[3,4-b]pyridin-4-yl)-2-(5-fluoropyridin-2-yl)-5, 5a,6,6a-tetrahydro-4H-cyclopropa[e]pyrazolo[1,5-a]pyridine;
(5a*S,6a*R)-6, 6-Difluoro-3-(5-fluoro-3-methyl-1H-pyrazolo[3,4-b]pyridin-4-yl)-2-(5-fluoropyridin-2-yl)-5, 5a,6,6a-tetrahydro-4H-cyclopropa[e]pyrazolo[1,5-a]pyridine;
(5a*S,6a*R)-6, 6-Difluoro-3-(5-fluoro-3,6-dimethyl-1H-pyrazolo[3,4-b]pyridin-4-yl)-2-(5-fluoropyridin-2-yl)-5, 5a,6,6a-tetrahydro-4H-cyclopropa[e]pyrazolo[1,5-a]pyridine;
(5a*S,6a*R)-6, 6-Difluoro-2-(5-fluoropyridin-2-yl)-3-(6-methyl-1H-pyrazolo[3,4-d]pyrimidin-4-yl)-5,5a,6,6a-tetrahydro-4H-cyclopropa[e]pyrazolo[1,5-a]pyridine;
(4a*R, 5a*R)-5, 5-Difluoro-2-(4-fluorophenyl)-3-(1H-pyrazolo[3,4-b]pyridin-4-yl)-4a,5,5a,6-tetrahydro-4H-cyclopropa[d]pyrazolo[1,5-a]pyridine;
(4a*S,5a*S)-5,5-Difluoro-2-(4-fluorophenyl)-3-(1H-pyrazolo[3,4-b]pyridin-4-yl)-4a,5,5a,6-tetrahydro-4H-cyclopropa[d]pyrazolo[1,5-a]pyridine;
(4*R,7*S)-2-(4-Fluorophenyl)-3-(6-methyl-1H-pyrazolo[3,4-b]pyridin-4-yl)-4,5,6,7-tetrahydro-4,7-methanopyrazolo[1,5-a]pyridine;
(4*S,7*R)-2-(4-Fluorophenyl)-3-(6-methyl-1H-pyrazolo[3,4-b]pyridin-4-yl)-4,5,6,7-tetrahydro-4,7-methanopyrazolo[1,5-a]pyridine;
2-(5-Fluoropyridin-2-yl)-6,6-bis(methyl-d₃)-3-(1H-pyrazolo[3,4-b]pyridin-4-yl)-4,5,6,7-tetrahydropyrazolo[1,5-a]pyridin-4-ol;
6-(5-Fluoro-2-pyridyl)-2,2-dimethyl-7-pyrazolo[1,5-a]pyridin-5-yl-3H-pyrazolo[5,1-b]oxazole;
6-(5-Fluoro-2-pyridyl)-2,2-dimethyl-7-(6-methyl-1H-pyrazolo[3,4-b]pyridin-4-yl)-3H-pyrazolo[5,1-b]oxazole;
2-(4-Fluorophenyl)-3-(4-pyridyl)-6,7-dihydro-4H-pyrazolo[5,1-c][1,4]oxazine;
3-(1-Ethyl-1H-pyrazolo[3,4-b]pyridin-5-yl)-2-(4-fluorophenyl)-7-methyl-6,7-dihydro-4H-pyrazolo[5,1-c][1,4]oxazine;
(S)-3-(1-Ethyl-1H-pyrazolo[3,4-b]pyridin-5-yl)-2-(5-fluoropyridin-2-yl)-6-methyl-6,7-dihydro-4H-pyrazolo[5,1-c][1,4]oxazine;
(*S)-2-(4-Fluorophenyl)-3-(3-methyl-H-pyrazolo[3,4-b]pyridin-4-yl)-6-(trifluoromethyl)-6,7-dihydro-4H-pyrazolo[5,1-c][1,4]oxazine;
(*R)-2-(4-Fluorophenyl)-3-(3-methyl-1H-pyrazolo[3,4-b]pyridin-4-yl)-6-(trifluoromethyl)-6,7-dihydro-4H-pyrazolo[5,1-c][1,4]oxazine;
(*S)-3-[6-(Difluoromethyl)-3-methyl-H-pyrazolo[3,4-b]pyridin-4-yl]-2-(4-fluorophenyl)-6-(trifluoromethyl)-6,7-dihydro-4H-pyrazolo[5,1-c][1,4]oxazine;
(*R)-3-[6-(Difluoromethyl)-3-methyl-1H-pyrazolo[3,4-b]pyridin-4-yl]-2-(4-fluorophenyl)-6-(trifluoromethyl)-6,7-dihydro-4H-pyrazolo[5,1-c][1,4]oxazine;
(*R)-2-(4-Fluorophenyl)-3-(6-methyl-1H-pyrazolo[3,4-d]pyrimidin-4-yl)-6-(trifluoromethyl)-6,7-dihydro-4H-pyrazolo[5,1-c][1,4]oxazine;
(*S)-2-(4-Fluorophenyl)-3-(6-methyl-1H-pyrazolo[3,4-d]pyrimidin-4-yl)-6-(trifluoromethyl)-6,7-dihydro-4H-pyrazolo[5,1-c][1,4]oxazine;
4-(2-(5-Fluoropyridin-2-yl)-6,6-dimethyl-6,7-dihydro-4H-pyrazolo[5,1-c][1,4]oxazin-3-yl)-5-methylpyridin-2-amine;
N-(4-(2-(5-Fluoropyridin-2-yl)-6,6-dimethyl-6,7-dihydro-4H-pyrazolo[5,1-c][1,4]oxazin-3-yl)-5-methylpyridin-2-yl)propionamide;
5-[2-(5-Fluoro-2-pyridyl)-6,6-dimethyl-4,7-dihydropyrazolo[5,1-c][1,4]oxazin-3-yl]pyrazolo[1,5-a]pyridin-7-amine;
5-[2-(5-Fluoro-2-pyridyl)-6,6-dimethyl-4,7-dihydropyrazolo[5,1-c][1,4]oxazin-3-yl]pyrazolo[1,5-a]pyridin-3-amine;
2-(5-Fluoro-2-pyridyl)-6,6-dimethyl-3-(6-methylpyrazolo[1,5-a]pyridin-5-yl)-4,7-dihydropyrazolo[5,1-c][1,4]oxazine;
2-(5-Fluoro-2-pyridyl)-6,6-dimethyl-3-(3-methylpyrazolo[1,5-a]pyridin-5-yl)-4,7-dihydropyrazolo[5,1-c][1,4]oxazine;
3-(3-Chloropyrazolo[1,5-a]pyridin-5-yl)-2-(5-fluoropyridin-2-yl)-6,6-dimethyl-6,7-dihydro-4H-pyrazolo[5,1-c][1,4]oxazine;
3-([1,2,4]Triazolo[1,5-a]pyridin-7-yl)-2-(5-fluoropyridin-2-yl)-6,6-dimethyl-6,7-dihydro-4H-pyrazolo[5,1-c][1,4]oxazine;
2-(5-Fluoropyridin-2-yl)-6,6-dimethyl-3-(2-methyl-[1,2,4]triazolo[1,5-a]pyridin-7-yl)-6,7-dihydro-4H-pyrazolo[5,1-c][1,4]oxazine;
2-(5-Fluoropyridin-2-yl)-6,6-dimethyl-3-(pyrazolo[1,5-a]pyrimidin-5-yl)-6,7-dihydro-4H-pyrazolo[5,1-c][1,4]oxazine;
3-(3-Chloro-1H-pyrazolo[3,4-b]pyridin-4-yl)-2-(5-fluoropyridin-2-yl)-6,6-dimethyl-6,7-dihydro-4H-pyrazolo[5,1-c][1,4]oxazine;
3-(5-Fluoro-1H-pyrazolo[3,4-b]pyridin-4-yl)-2-(5-fluoropyridin-2-yl)-6,6-dimethyl-6,7-dihydro-4H-pyrazolo[5,1-c][1,4]oxazine;
3-[6-(Difluoromethyl)-1H-pyrazolo[3,4-b]pyridin-4-yl]-2-(5-fluoro-2-pyridyl)-6,6-dimethyl-4,7-dihydropyrazolo[5,1-c][1,4]oxazine;
2-(5-Fluoro-2-pyridyl)-6,6-dimethyl-3-[6-(3,3,3-trifluoropropyl)-1H-pyrazolo[3,4-b]pyridin-4-yl]-4,7-dihydropyrazolo[5,1-c][1,4]oxazine;
3-(3-Chloro-5-fluoro-1H-pyrazolo[3,4-b]pyridin-4-yl)-2-(5-fluoropyridin-2-yl)-6,6-dimethyl-6,7-dihydro-4H-pyrazolo[5,1-c][1,4]oxazine;
3-(3,6-Dimethyl-1H-pyrazolo[3,4-b]pyridin-4-yl)-2-(5-fluoro-6-methylpyridin-2-yl)-6,6-dimethyl-6,7-dihydro-4H-pyrazolo[5,1-c][1,4]oxazine;
2-(4-Fluorophenyl)-6,6-dimethyl-3-(6-methyl-1H-pyrazolo[3,4-d]pyrimidin-4-yl)-4,7-dihydropyrazolo[5,1-c][1,4]oxazine;
N-(4-(2-(5-Fluoropyridin-2-yl)-6,6-bis(methyl-d₃)-6,7-dihydro-4H-pyrazolo[5,1-c][1,4]oxazin-3-yl)pyridin-2-yl)propionamide;
2-(5-Fluoropyridin-2-yl)-6,6-bis(methyl-d₃)-3-(pyrazolo[1,5-a]pyridin-5-yl)-6,7-dihydro-4H-pyrazolo[5,1-c][1,4]oxazine;
3-(5-Fluoro-1H-pyrrolo[2,3-b]pyridin-4-yl)-2-(5-fluoropyridin-2-yl)-6,6-bis(methyl-d₃)-6,7-dihydro-4H-pyrazolo[5,1-c][1,4]oxazine;
3-(3-Fluoro-1H-pyrazolo[3,4-b]pyridin-4-yl)-2-(5-fluoropyridin-2-yl)-6,6-bis(methyl-d₃)-6,7-dihydro-4H-pyrazolo[5,1-c][1,4]oxazine;
3-(5-Fluoro-1H-pyrazolo[3,4-b]pyridin-4-yl)-2-(5-fluoropyridin-2-yl)-6,6-bis(methyl-d₃)-6,7-dihydro-4H-pyrazolo[5,1-c][1,4]oxazine;
3-(3-Chloro-1H-pyrazolo[3,4-b]pyridin-4-yl)-2-(5-fluoropyridin-2-yl)-6,6-bis(methyl-d₃)-6,7-dihydro-4H-pyrazolo[5,1-c][1,4]oxazine;
2-(5-Fluoropyridin-2-yl)-6,6-bis(methyl-d₃)-3-(6-methyl-1H-pyrazolo[3,4-b]pyridin-4-yl)-6,7-dihydro-4H-pyrazolo[5,1-c][1,4]oxazine;
3-(6-(Difluoromethyl)-1H-pyrazolo[3,4-b]pyridin-4-yl)-2-(5-fluoropyridin-2-yl)-6,6-bis(methyl-d₃)-6,7-dihydro-4H-pyrazolo[5,1-c][1,4]oxazine;
3-(3-Chloro-5-fluoro-1H-pyrazolo[3,4-b]pyridin-4-yl)-2-(5-fluoropyridin-2-yl)-6,6-bis(methyl-d₃)-6,7-dihydro-4H-pyrazolo[5,1-c][1,4]oxazine;
3-(5-Fluoro-3-methyl-1H-pyrazolo[3,4-b]pyridin-4-yl)-2-(5-fluoropyridin-2-yl)-6,6-bis(methyl-d₃)-6,7-dihydro-4H-pyrazolo[5,1-c][1,4]oxazine;
3-(3-Chloro-6-methyl-1H-pyrazolo[3,4-b]pyridin-4-yl)-2-(5-fluoropyridin-2-yl)-6,6-bis(methyl-d₃)-6,7-dihydro-4H-pyrazolo[5,1-c][1,4]oxazine;
3-(5-Fluoro-6-methyl-1H-pyrazolo[3,4-b]pyridin-4-yl)-2-(5-fluoropyridin-2-yl)-6,6-bis(methyl-d₃)-6,7-dihydro-4H-pyrazolo[5,1-c][1,4]oxazine;
3-(5-Chloro-6-methyl-1H-pyrazolo[3,4-b]pyridin-4-yl)-2-(5-fluoropyridin-2-yl)-6,6-bis(methyl-d₃)-6,7-dihydro-4H-pyrazolo[5,1-c][1,4]oxazine;
3-(3-Chloro-5-fluoro-6-methyl-1H-pyrazolo[3,4-b]pyridin-4-yl)-2-(5-fluoropyridin-2-yl)-6,6-bis(methyl-d₃)-6,7-dihydro-4H-pyrazolo[5,1-c][1,4]oxazine;
3-(5-Fluoro-3,6-dimethyl-1H-pyrazolo[3,4-b]pyridin-4-yl)-2-(5-fluoropyridin-2-yl)-6,6-bis(methyl-d₃)-6,7-dihydro-4H-pyrazolo[5,1-c][1,4]oxazine;
2-(4-Fluorophenyl)-6,6-bis(methyl-d₃)-3-(6-methyl-1H-pyrazolo[3,4-d]pyrimidin-4-yl)-6,7-dihydro-4H-pyrazolo[5,1-c][1,4]oxazine;
2-(5-Fluoropyridin-2-yl)-6,6-bis(methyl-d₃)-3-(1H-pyrazolo[3,4-b]pyridin-4-yl)-6,7-dihydro-4H-pyrazolo[5,1-c][1,4]oxazine-4,4,7,7-d₄;
(*R)-2-(4-Fluorophenyl)-6-methyl-3-(pyrazolo[1,5-a]pyridin-5-yl)-6-(trifluoromethyl)-6,7-dihydro-4H-pyrazolo[5,1-c][1,4]oxazine;
(*S)-2-(4-Fluorophenyl)-6-methyl-3-(pyrazolo[1,5-a]pyridin-5-yl)-6-(trifluoromethyl)-6,7-dihydro-4H-pyrazolo[5,1-c][1,4]oxazine;
(*R)-2-(5-Fluoropyridin-2-yl)-6-methyl-3-(pyrazolo[1,5-a]pyridin-5-yl)-6-(trifluoromethyl)-6,7-dihydro-4H-pyrazolo[5,1-c][1,4]oxazine;
(*S)-2-(5-Fluoropyridin-2-yl)-6-methyl-3-(pyrazolo[1,5-a]pyridin-5-yl)-6-(trifluoromethyl)-6,7-dihydro-4H-pyrazolo[5,1-c][1,4]oxazine;
(*R)-2-(4-Fluorophenyl)-6-methyl-3-(1H-pyrazolo[3,4-b]pyridin-4-yl)-6-(trifluoromethyl)-6,7-dihydro-4H-pyrazolo[5,1-c][1,4]oxazine;
(*S)-2-(4-Fluorophenyl)-6-methyl-3-(1H-pyrazolo[3,4-b]pyridin-4-yl)-6-(trifluoromethyl)-6,7-dihydro-4H-pyrazolo[5,1-c][1,4]oxazine;
(*R)-2-(5-Fluoropyridin-2-yl)-6-methyl-3-(3-methyl-1H-pyrazolo[3,4-b]pyridin-4-yl)-6-(trifluoromethyl)-6,7-dihydro-4H-pyrazolo[5,1-c][1,4]oxazine;
(*S)-2-(5-Fluoropyridin-2-yl)-6-methyl-3-(3-methyl-H-pyrazolo[3,4-b]pyridin-4-yl)-6-(trifluoromethyl)-6,7-dihydro-4H-pyrazolo[5,1-c][1,4]oxazine;
(*R)-3-(5-Fluoro-6-methyl-1H-pyrazolo[3,4-b]pyridin-4-yl)-2-(5-fluoropyridin-2-yl)-6-methyl-6-(trifluoromethyl)-6,7-dihydro-4H-pyrazolo[5,1-c][1,4]oxazine;
(*S)-3-(5-Fluoro-6-methyl-1H-pyrazolo[3,4-b]pyridin-4-yl)-2-(5-fluoropyridin-2-yl)-6-methyl-6-(trifluoromethyl)-6,7-dihydro-4H-pyrazolo[5,1-c][1,4]oxazine;
2-(4-Fluorophenyl)-6-methyl-3-(6-methyl-1H-pyrazolo[3,4-d]pyrimidin-4-yl)-6-(trifluoromethyl)-6,7-dihydro-4H-pyrazolo[5,1-c][1,4]oxazine;
(*R)-2-(5-Fluoropyridin-2-yl)-6-(methyl-d₃)-3-(1H-pyrazolo[3,4-b]pyridin-4-yl)-6-(trifluoromethyl)-6,7-dihydro-4H-pyrazolo[5,1-c][1,4]oxazine;
(*S)-2-(5-Fluoropyridin-2-yl)-6-(methyl-d₃)-3-(1H-pyrazolo[3,4-b]pyridin-4-yl)-6-(trifluoromethyl)-6,7-dihydro-4H-pyrazolo[5,1-c][1,4]oxazine;
(*R)-2-(5-Fluoropyridin-2-yl)-6-(methyl-d₃)-3-(3-methyl-1H-pyrazolo[3,4-b]pyridin-4-yl)-6-(trifluoromethyl)-6,7-dihydro-4H-pyrazolo[5,1-c][1,4]oxazine;
(*S)-2-(5-Fluoropyridin-2-yl)-6-(methyl-d₃)-3-(3-methyl-H-pyrazolo[3,4-b]pyridin-4-yl)-6-(trifluoromethyl)-6,7-dihydro-4H-pyrazolo[5,1-c][1,4]oxazine;
(*S)-3-(3-Chloro-1H-pyrazolo[3,4-b]pyridin-4-yl)-2-(5-fluoropyridin-2-yl)-6-(methyl-d₃)-6-(trifluoromethyl)-6,7-dihydro-4H-pyrazolo[5,1-c][1,4]oxazine;
(*R)-3-(3,6-Dimethyl-1H-pyrazolo[3,4-b]pyridin-4-yl)-2-(5-fluoropyridin-2-yl)-6-(methyl-d₃)-6-(trifluoromethyl)-6,7-dihydro-4H-pyrazolo[5,1-c][1,4]oxazine;
(*S)-3-(3,6-Dimethyl-1H-pyrazolo[3,4-b]pyridin-4-yl)-2-(5-fluoropyridin-2-yl)-6-(methyl-d₃)-6-(trifluoromethyl)-6,7-dihydro-4H-pyrazolo[5,1-c][1,4]oxazine;
(*R)-3-(5-Fluoro-3,6-dimethyl-1H-pyrazolo[3,4-b]pyridin-4-yl)-2-(5-fluoropyridin-2-yl)-6-(methyl-d₃)-6-(trifluoromethyl)-6,7-dihydro-4H-pyrazolo[5,1-c][1,4]oxazine;
(*S)-3-(5-Fluoro-3, 6-dimethyl-1H-pyrazolo[3,4-b]pyridin-4-yl)-2-(5-fluoropyridin-2-yl)-6-(methyl-d₃)-6-(trifluoromethyl)-6,7-dihydro-4H-pyrazolo[5,1-c][1,4]oxazine;
2-(5-Fluoro-2-pyridyl)-3-(1H-pyrazolo[3,4-b]pyridin-4-yl)spiro[4,7-dihydropyrazolo[5,1-c][1,4]oxazine-6,1′-cyclopropane];
(*R)-1′,1′-Difluoro-2-(5-fluoro-2-pyridyl)-3-(1H-pyrazolo[3,4-b]pyridin-4-yl)spiro[4,7-dihydropyrazolo[5,1-c][1,4]oxazine-6,2′-cyclopropane];
(*S)-1′,1′-Difluoro-2-(5-fluoro-2-pyridyl)-3-(1H-pyrazolo[3,4-b]pyridin-4-yl)spiro[4,7-dihydropyrazolo[5,1-c][1,4]oxazine-6,2′-cyclopropane];
(*R)-1′,1′-Difluoro-3-(5-fluoro-1H-pyrazolo[3,4-b]pyridin-4-yl)-2-(5-fluoro-2-pyridyl)spiro[4,7-dihydropyrazolo[5,1-c][1,4]oxazine-6,2′-cyclopropane];
(*S)-1′,1′-Difluoro-3-(5-fluoro-1H-pyrazolo[3,4-b]pyridin-4-yl)-2-(5-fluoro-2-pyridyl)spiro[4,7-dihydropyrazolo[5,1-c][1,4]oxazine-6,2′-cyclopropane];
(*S)-3-(3-Chloro-1H-pyrazolo[3,4-b]pyridin-4-yl)-2-(5-fluoropyridin-2-yl)-6,6,7-trimethyl-6,7-dihydro-4H-pyrazolo[5,1-c][1,4]oxazine;
2-(4-Fluorophenyl)-3-(4-pyridyl)-6,7-dihydro-5H-pyrazolo[5,1-b][1,3]oxazine;
N-(4-(2-(5-Fluoropyridin-2-yl)-6,7-dihydro-5H-pyrazolo[5,1-b][1,3]oxazin-3-yl)pyridin-2-yl)propionamide;
2-(5-Fluoropyridin-2-yl)-3-(pyrazolo[1,5-a]pyridin-5-yl)-6,7-dihydro-5H-pyrazolo[5,1-b][1,3]oxazine;
3-(1-Ethyl-1H-pyrazolo[3,4-b]pyridin-5-yl)-2-(5-fluoropyridin-2-yl)-6,7-dihydro-5H-pyrazolo[5,1-b][1,3]oxazine;
N-(4-(2-(5-Fluoropyridin-2-yl)-6-methyl-6,7-dihydro-5H-pyrazolo[5,1-b][1,3]oxazin-3-yl)pyridin-2-yl)propionamide;
2-(5-Fluoropyridin-2-yl)-6-methyl-3-(pyrazolo[1,5-a]pyridin-5-yl)-6,7-dihydro-5H-pyrazolo[5,1-b][1,3]oxazine;
3-(1-Ethyl-1H-pyrazolo[3,4-b]pyridin-5-yl)-2-(5-fluoropyridin-2-yl)-6-methyl-6,7-dihydro-5H-pyrazolo[5,1-b][1,3]oxazine;
N-(4-(2-(5-Fluoropyridin-2-yl)-7-methyl-6,7-dihydro-5H-pyrazolo[5,1-b][1,3]oxazin-3-yl)pyridin-2-yl)propionamide;
2-(5-Fluoropyridin-2-yl)-7-methyl-3-(pyrazolo[1,5-a]pyridin-5-yl)-6,7-dihydro-5H-pyrazolo[5,1-b][1,3]oxazine;
N-(4-(2-(5-Fluoropyridin-2-yl)-6,6-dimethyl-6,7-dihydro-5H-pyrazolo[5,1-b][1,3]oxazin-3-yl)pyridin-2-yl)propionamide;
2-(4-Fluorophenyl)-6,6-dimethyl-3-(1H-pyrrolo[3,2-b]pyridin-7-yl)-5,7-dihydropyrazolo[5,1-b][1,3]oxazine;
2-(5-Fluoropyridin-2-yl)-6,6-dimethyl-3-(pyrazolo[1,5-a]pyridin-5-yl)-6,7-dihydro-5H-pyrazolo[5,1-b][1,3]oxazine;
2-(5-Fluoro-2-pyridyl)-6,6-dimethyl-3-(1H-pyrrolo[3,2-b]pyridin-7-yl)-5,7-dihydropyrazolo[5,1-b][1,3]oxazine;
2-(5-Fluoro-2-pyridyl)-6,6-dimethyl-3-(6-methylpyrazolo[1,5-a]pyridin-5-yl)-5,7-dihydropyrazolo[5,1-b][1,3]oxazine;
3-(1-Ethyl-1H-pyrazolo[3,4-b]pyridin-5-yl)-2-(5-fluoropyridin-2-yl)-6,6-dimethyl-6,7-dihydro-5H-pyrazolo[5,1-b][1,3]oxazine;
2-(4-Fluorophenyl)-6,6-dimethyl-3-(1H-pyrazolo[4,3-b]pyridin-7-yl)-5,7-dihydropyrazolo[5,1-b][1,3]oxazine;
2-(5-Fluoro-2-pyridyl)-6,6-dimethyl-3-(1H-pyrazolo[4,3-b]pyridin-7-yl)-5,7-dihydropyrazolo[5,1-b][1,3]oxazine;
N-(4-(2′-(4-Fluorophenyl)-5′H,7′H-spiro[cyclopropane-1,6′-pyrazolo[5,1-b][1,3]oxazin]-3′-yl)pyridin-2-yl)propionamide;
N-(4-(2′-(5-Fluoropyridin-2-yl)-5′H,7′H-spiro[cyclopropane-1,6′-pyrazolo[5,1-b][1,3]oxazin]-3′-yl)pyridin-2-yl)cyclopropanecarboxamide;
2′-(4-Fluorophenyl)-3′-(1H-pyrazolo[3,4-b]pyridin-4-yl)-5′H,7′H-spiro[cyclopropane-1,6′-pyrazolo[5,1-b][1,3]oxazine];
3′-(3-Chloro-1H-pyrazolo[3,4-b]pyridin-4-yl)-2′-(5-fluoropyridin-2-yl)-5′H,7′H-spiro[cyclopropane-1,6′-pyrazolo[5,1-b][1,3]oxazine];
3′-(5-Fluoro-1H-pyrazolo[3,4-b]pyridin-4-yl)-2′-(5-fluoropyridin-2-yl)-5′H,7′H-spiro[cyclopropane-1,6′-pyrazolo[5,1-b][1,3]oxazine];
2′-(5-Fluoropyridin-2-yl)-3′-(1H-pyrazolo[3,4-b]pyridin-4-yl)-5′H,7′H-spiro[cyclopropane-1,6′-pyrazolo[5,1-b][1,3]oxazine];
2′-(4-Fluorophenyl)-3′-(6-methyl-1H-pyrazolo[3,4-b]pyridin-4-yl)-5′H,7′H-spiro[cyclopropane-1,6′-pyrazolo[5,1-b][1,3]oxazine];
2′-(5-Fluoropyridin-2-yl)-3′-(6-methyl-1H-pyrazolo[3,4-b]pyridin-4-yl)-5′H,7′H-spiro[cyclopropane-1,6′-pyrazolo[5,1-b][1,3]oxazine];
3′-(5-Fluoro-6-methyl-1H-pyrazolo[3,4-b]pyridin-4-yl)-2′-(5-fluoropyridin-2-yl)-5′H,7′H-spiro[cyclopropane-1,6′-pyrazolo[5,1-b][1,3]oxazine];
3′-(3-Chloro-6-methyl-1H-pyrazolo[3,4-b]pyridin-4-yl)-2′-(5-fluoropyridin-2-yl)-5′H,7′H-spiro[cyclopropane-1,6′-pyrazolo[5,1-b][1,3]oxazine];
2′-(4-Fluorophenyl)-3′-(6-methyl-1H-pyrazolo[3,4-d]pyrimidin-4-yl)-5′H,7′H-spiro[cyclopropane-1,6′-pyrazolo[5,1-b][1,3]oxazine];
2′-(4-Fluorophenyl)-3′-(pyrazolo[1,5-a]pyridin-5-yl)-5′H,7′H-spiro[cyclopropane-1,6′-pyrazolo[5,1-b][1,3]oxazine];
6,6-Difluoro-2-(4-fluorophenyl)-3-(1H-pyrazolo[3,4-b]pyridin-4-yl)-5,7-dihydropyrazolo[5,1-b][1,3]oxazine;
6,6-Difluoro-2-(4-fluorophenyl)-3-(1H-pyrazolo[4,3-b]pyridin-7-yl)-5,7-dihydropyrazolo[5,1-b][1,3]oxazine;
6,6-Difluoro-2-(4-fluorophenyl)-3-(6-methyl-1H-pyrazolo[3,4-d]pyrimidin-4-yl)-5,7-dihydropyrazolo[5,1-b][1,3]oxazine;
3-(3-Chloro-1H-pyrazolo[3,4-b]pyridin-4-yl)-6,6-difluoro-2-(4-fluorophenyl)-6,7-dihydro-5H-pyrazolo[5,1-b][1,3]oxazine;
2,2-Difluoro-3′-(5-fluoro-1H-pyrazolo[3,4-b]pyridin-4-yl)-2′-(4-fluorophenyl)-5′H,7′H-spiro[cyclopropane-1,6′-pyrazolo[5,1-b][1,3]oxazine];
(*S)-2,2-Difluoro-2′-(4-fluorophenyl)-3′-(3-methyl-1H-pyrazolo[3,4-b]pyridin-4-yl)-5′H,7′H-spiro[cyclopropane-1,6′-pyrazolo[5,1-b][1,3]oxazine];
(*R)-2,2-Difluoro-2′-(4-fluorophenyl)-3′-(3-methyl-H-pyrazolo[3,4-b]pyridin-4-yl)-5′H,7′H-spiro[cyclopropane-1,6′-pyrazolo[5,1-b][1,3]oxazine];
(*S)-2,2-Difluoro-2′-(4-fluorophenyl)-3′-(6-methyl-1H-pyrazolo[3,4-b]pyridin-4-yl)-5′H,7′H-spiro[cyclopropane-1,6′-pyrazolo[5,1-b][1,3]oxazine];
(*R)-2,2-Difluoro-2′-(4-fluorophenyl)-3′-(6-methyl-H-pyrazolo[3,4-b]pyridin-4-yl)-5′H,7′H-spiro[cyclopropane-1,6′-pyrazolo[5,1-b][1,3]oxazine];
(*S)-2,2-Difluoro-2′-(4-fluorophenyl)-3′-(1H-pyrazolo[3,4-b]pyridin-4-yl)-5′H,7′H-spiro[cyclopropane-1,6′-pyrazolo[5,1-b][1,3]oxazine];
(*R)-2,2-Difluoro-2′-(4-fluorophenyl)-3′-(1H-pyrazolo[3,4-b]pyridin-4-yl)-5′H,7′H-spiro[cyclopropane-1,6′-pyrazolo[5,1-b][1,3]oxazine];
(*S)-2,2-Difluoro-2′-(5-fluoropyridin-2-yl)-3′-(1H-pyrazolo[3,4-b]pyridin-4-yl)-5′H,7′H-spiro[cyclopropane-1,6′-pyrazolo[5,1-b][1,3]oxazine];
(*R)-2,2-Difluoro-2′-(5-fluoropyridin-2-yl)-3′-(1H-pyrazolo[3,4-b]pyridin-4-yl)-5′H,7′H-spiro[cyclopropane-1,6′-pyrazolo[5,1-b][1,3]oxazine];
(5a*R, 6a*S)-3-(5-Fluoro-1H-pyrazolo[3,4-b]pyridin-4-yl)-2-(5-fluoropyridin-2-yl)-5a,6,6a,7-tetrahydro-5H-cyclopropa[e]pyrazolo[5,1-b][1,3]oxazepine;
(5a*S,6a*R)-3-(5-Fluoro-1H-pyrazolo[3,4-b]pyridin-4-yl)-2-(5-fluoropyridin-2-yl)-5a,6,6a,7-tetrahydro-5H-cyclopropa[e]pyrazolo[5,1-b][1,3]oxazepane;
(5a*R, 6a*S)-3-(5-Fluoro-6-methyl-1H-pyrazolo[3,4-b]pyridin-4-yl)-2-(5-fluoropyridin-2-yl)-5a,6,6a,7-tetrahydro-5H-cyclopropa[e]pyrazolo[5,1-b][1,3]oxazepine;
(5a*S,6a*R)-3-(5-Fluoro-6-methyl-1H-pyrazolo[3,4-b]pyridin-4-yl)-2-(5-fluoropyridin-2-yl)-5a,6,6a,7-tetrahydro-5H-cyclopropa[e]pyrazolo[5,1-b][1,3]oxazepine;
2-(5-Fluoropyridin-2-yl)-7-methyl-3-(1H-pyrazolo[3,4-b]pyridin-4-yl)-7,8-dihydro-4H,6H-pyrazolo[5,1-c][1,4]oxazepin-7-ol;
(*S)-2-(5-Fluoropyridin-2-yl)-7-methoxy-7-methyl-3-(1H-pyrazolo[3,4-b]pyridin-4-yl)-7,8-dihydro-4H,6H-pyrazolo[5,1-c][1,4]oxazepane; or
(*R)-2-(5-Fluoropyridin-2-yl)-7-methoxy-7-methyl-3-(1H-pyrazolo[3,4-b]pyridin-4-yl)-7,8-dihydro-4H,6H-pyrazolo[5,1-c][1,4]oxazepine;
and pharmaceutically acceptable salts, isotopes, N-oxides, solvates, and stereoisomers thereof.

25. The compound of claim 1, wherein the compound is
2-(5-Fluoropyridin-2-yl)-6,6-dimethyl-3-(1H-pyrazolo[3,4-b]pyridin-4-yl)-6,7-dihydro-4H-pyrazolo[5,1-c][1,4]oxazine;
2-(5-Fluoropyridin-2-yl)-6,6-bis(methyl-d₃)-3-(1H-pyrazolo[3,4-b]pyridin-4-yl)-6,7-dihydro-4H-pyrazolo[5,1-c][1,4]oxazine;
5-Fluoro-4-(2-(5-fluoropyridin-2-yl)-5,6-dihydro-4H-pyrrolo[1,2-b]pyrazol-3-yl)-6-methyl-1H-pyrazolo[3,4-b]pyridine;
(*S)-4-(6-Fluoro-2-(5-fluoropyridin-2-yl)-6-(methoxymethyl)-4,5,6,7-tetrahydropyrazolo[1,5-a]pyridin-3-yl)-6-methyl-1H-pyrazolo[3,4-b]pyridine;
N-(4-(2-(5-Fluoropyridin-2-yl)-6,6-dimethyl-6,7-dihydro-4H-pyrazolo[5,1-c][1,4]oxazin-3-yl)pyridin-2-yl)propionamide;
2-(4-Fluorophenyl)-6,6-dimethyl-3-(6-methyl-1H-pyrazolo[3,4-b]pyridin-4-yl)-6,7-dihydro-5H-pyrazolo[5,1-b][1,3]oxazine; or
(4aS,5aS)-2-(5-Fluoropyridin-2-yl)-3-(1H-pyrazolo[3,4-b]pyridin-4-yl)-4,4a,5,5a-tetrahydrocyclopropa[4,5]pyrrolo[1,2-b]pyrazole;
and pharmaceutically acceptable salts, isotopes, N-oxides, solvates, and stereoisomers thereof.

26. The compound of claim 1, and pharmaceutically acceptable salts, isotopes, N-oxides, solvates, and stereoisomers thereof, having the structure of Formula (IA): 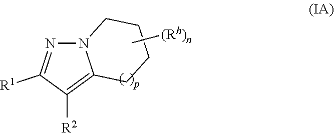
wherein
R¹ is selected from the group consisting of: 4-fluorophenyl, 5-fluoropyridin-2-yl, 3,5-difluoropyridin-2-yl, 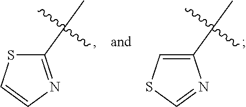 [SMILES: *c1nccs1 ; *c1cscn1]
R² is selected from the group consisting of: 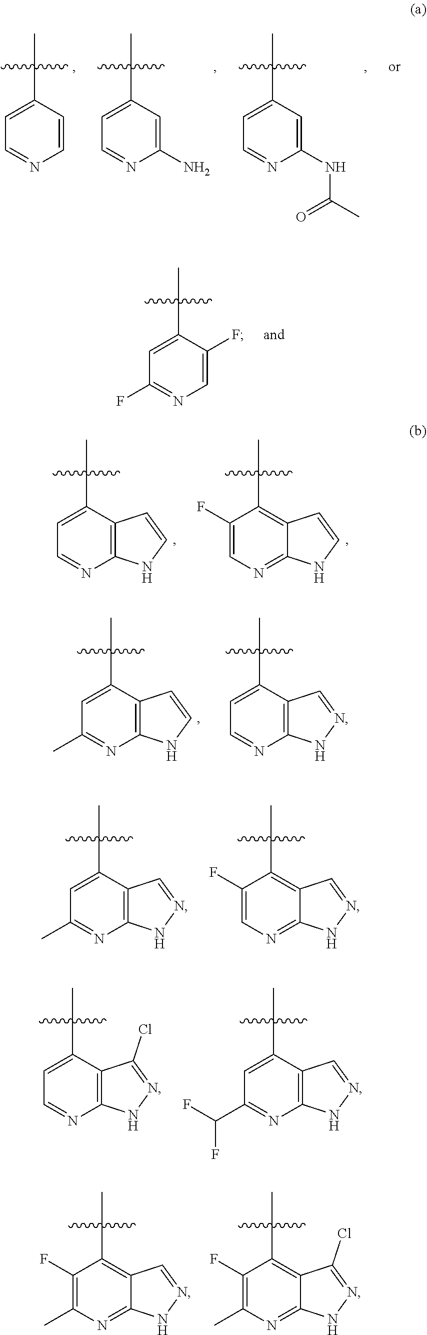 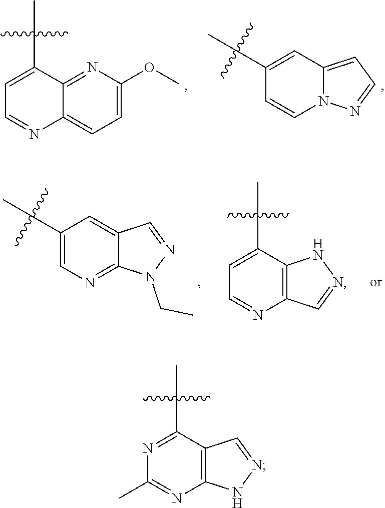
R^{h} is independently selected from the group consisting of: H, F, OH, CH₃, CD₃, CH₂F, CD₂F, CH₂OCH₃, CH₂OCD₃, CD₂₀CD₃, CH₂CH₂OCH₃, CH₂OCHF₂, CH₂OCF₃, CN, and 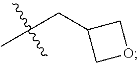 [SMILES: *CC1COC1]
n is 1, 2, or 3; and
p is 0 or 1.

27. The compound of claim 1, and pharmaceutically acceptable salts, isotopes, N-oxides, solvates, and stereoisomers thereof, having the structure of Formula (IB): 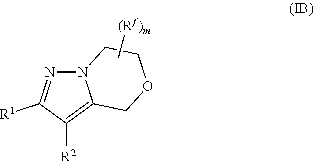
wherein
R¹ is selected from the group consisting of: 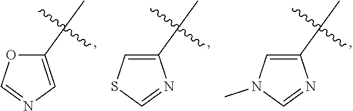
4-chlorophenyl, 4-fluorophenyl, 4-chloro-3-fluoro-phenyl, 5-fluoropyridin-2-yl, 5-chloropyridin-2-yl, 3-chloropyridin-4-yl, 5-fluoropyridin-3-yl, 3,5-difluoropyridin-2-yl, 3,5-difluoropyridin-4-yl, 5-fluoro-6-methyl-2-pyridyl, 6-methoxypyridin-2-yl, and 5-chloro-6-methylpyridin-2-yl;
R² is selected from the group consisting of: 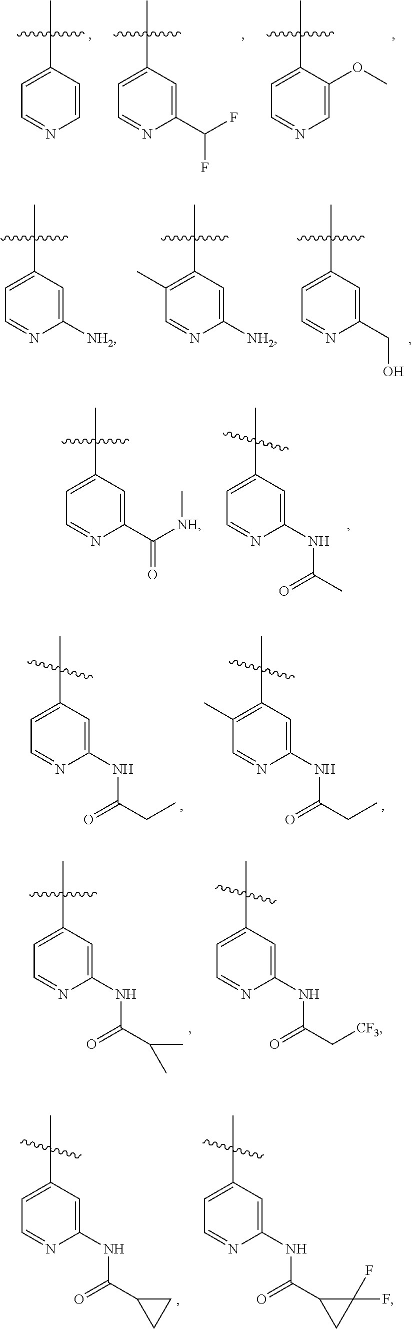 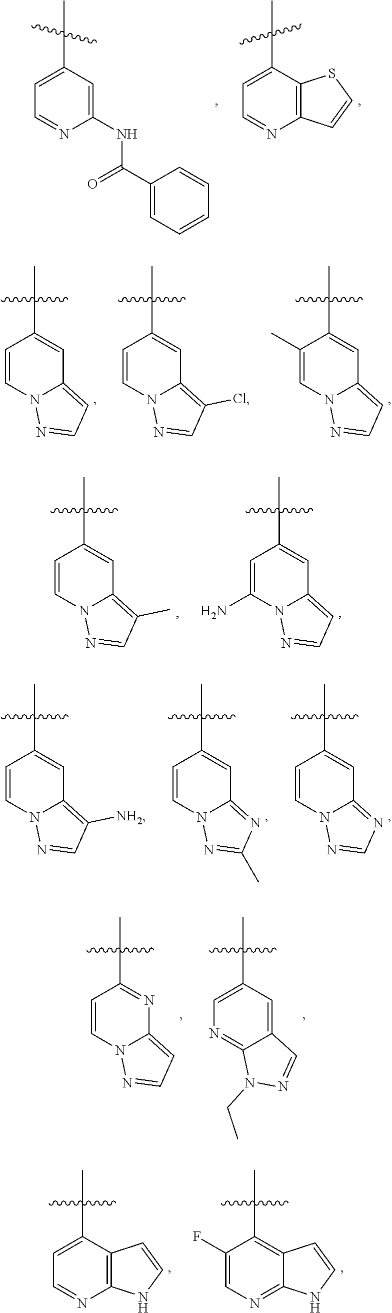 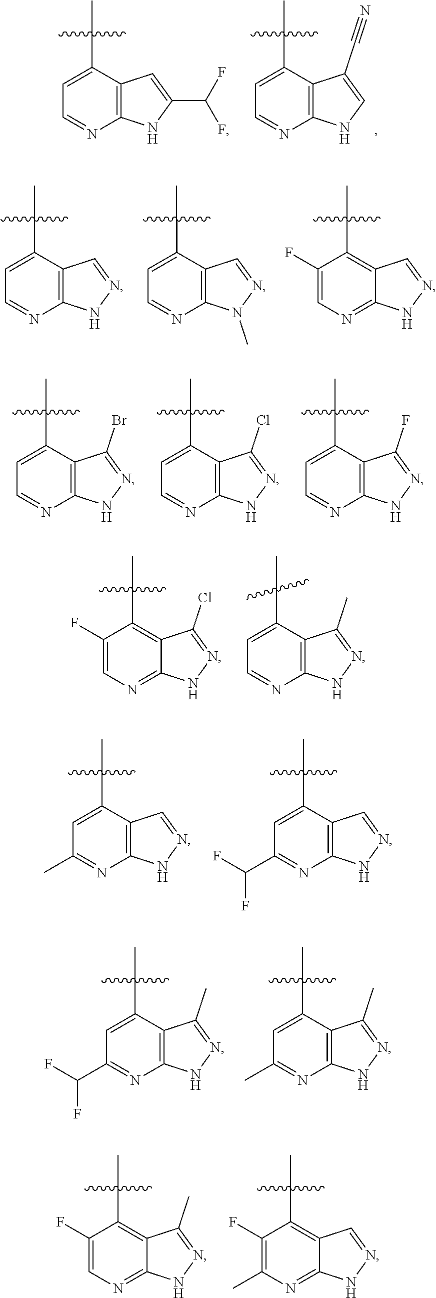 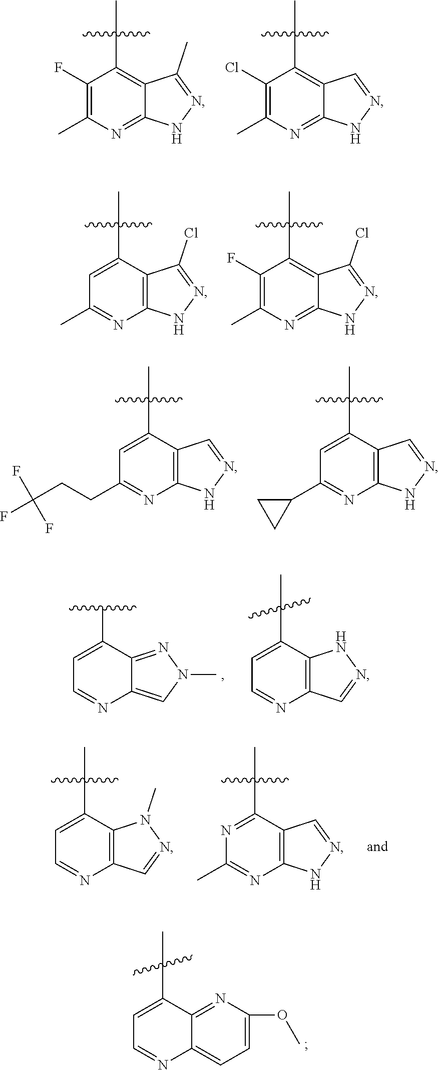
R^{f} is independently selected from the group consisting of: H, D, OH, CH₃, CD₃, CH₂CH₃, CH(CH₃)₂, CH₂F, CF₃, OCH₃, cyclopropyl, cyclobutyl, and two R^{f} members come together to form cyclopropyl wherein the cyclopropyl is optionally substituted with two F members; and
m is 1,2,3 or 4.

28. The compound of claim 1, and pharmaceutically acceptable salts, isotopes, N-oxides, solvates, and stereoisomers thereof, having the structure of Formula (IC): 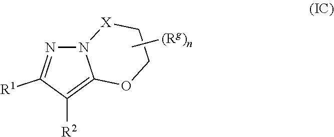
wherein
R¹ is selected from the group consisting of: 4-fluorophenyl, 5-fluoropyridin-2-yl, and 3,5-difluoropyridin-2-yl;
R² is selected from the group consisting of: 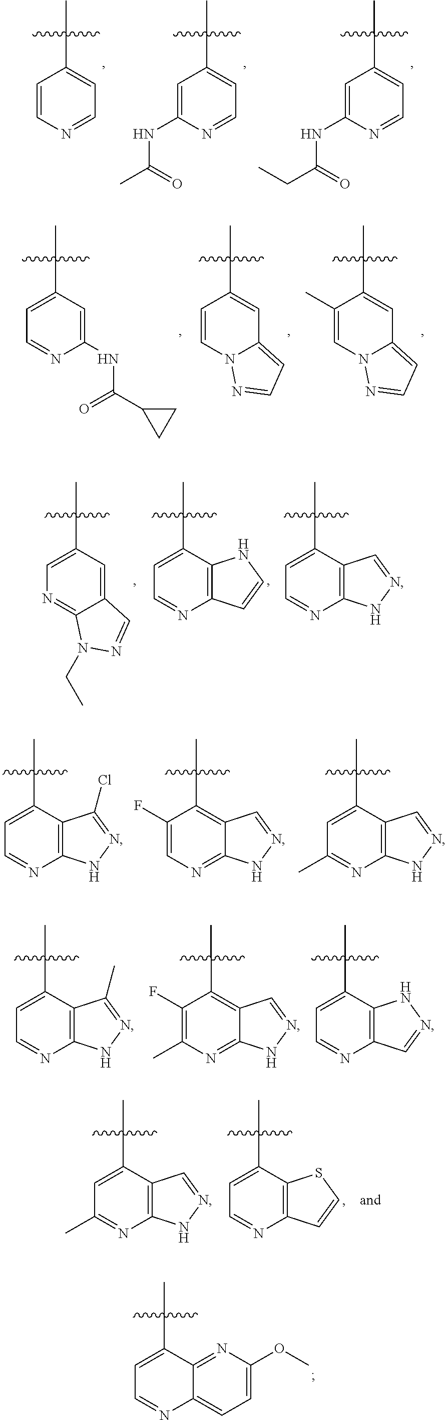
R^{g} is independently selected from the group consisting of: H, F, and CH₃;
X is selected from the group consisting of: a bond, CH₂, CH(CH₃), and CH₂CH₂; and
n is 1 or 2.

29. The compound of claim 1, and pharmaceutically acceptable salts, isotopes, N-oxides, solvates, and stereoisomers thereof, having the structure of Formula (ID): 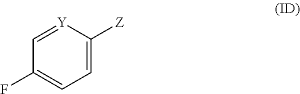
wherein
Y is CH or N; and
Z is selected from the group consisting of:

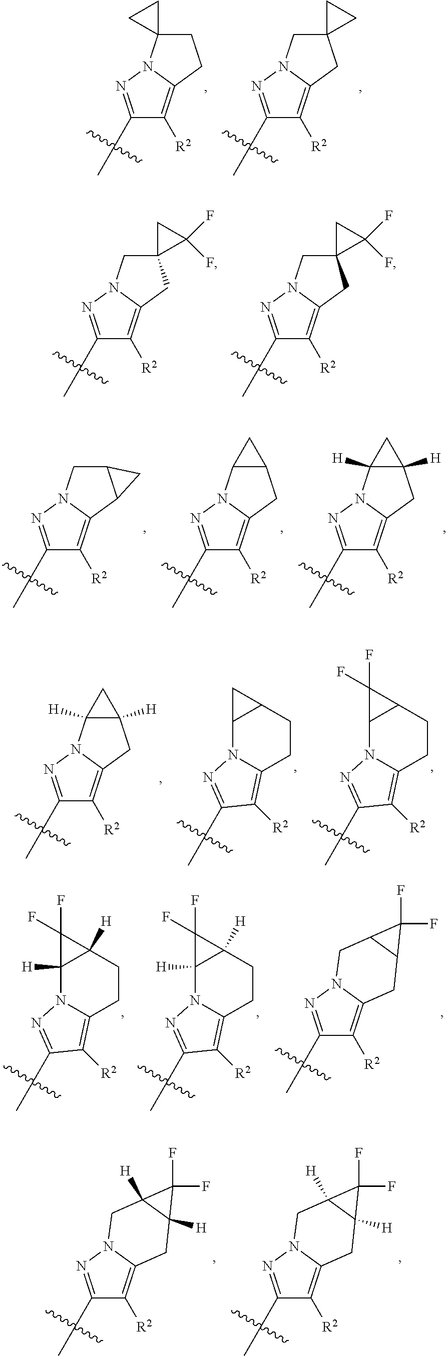

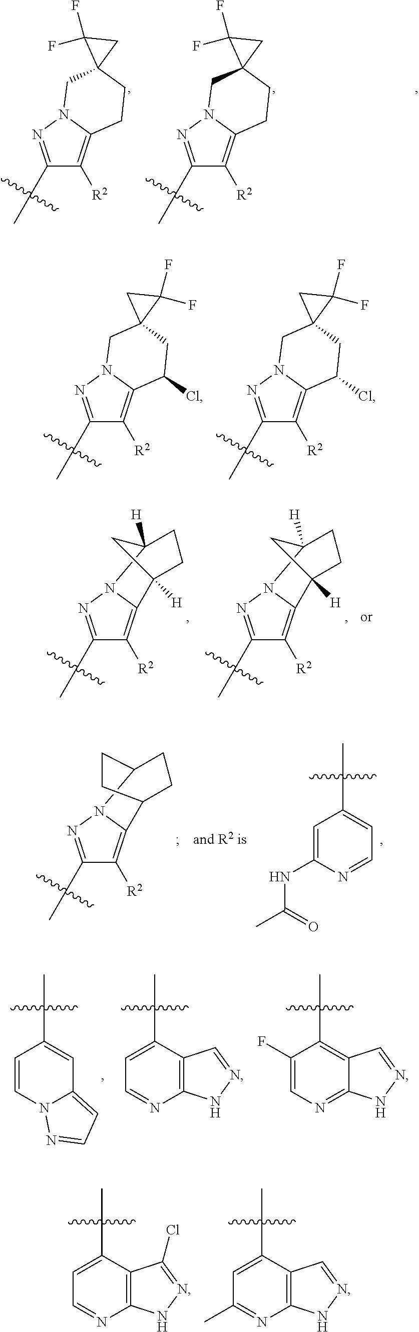

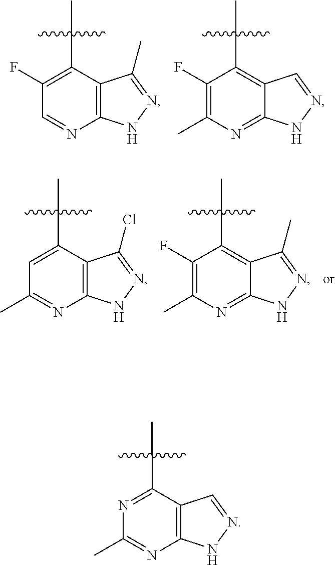

30. A pharmaceutical composition comprising:
(A) therapeutically effective amount of at least one compound of Formula (I): 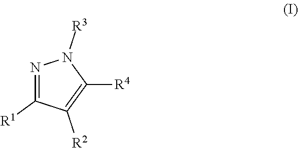
wherein
R¹ is selected from the group consisting of:
(a) phenyl substituted with one or two halo members;
(b) 5-fluoro-2-pyridyl optionally substituted with halo or C₁₋₃alkyl, 5-fluoro-3-pyridyl, 5-chloropyridin-2-yl, 3-chloropyridin-4-yl, 6-methoxypyridin-2-yl, 5-chloro-6-methylpyridin-2-yl, or 3,5-difluoropyridin-4-yl; and
(c) oxazol-5-yl, thiazol-2-yl, thiazol-4-yl, or 1-methyl-1H-imidazol-4-yl;
R² is selected from the group consisting of:
(d) 4-pyridyl optionally substituted with one member selected from the group consisting of: halo, C₁₋₃haloalkyl, CH₂OH, OC₁₋₃alkyl, (C═O)—NHCH₃, NH₂, NH—(C═O)C₁₋₃alkyl, NH—(C═O)C₁₋₃haloalkyl, NH—(C═O)phenyl, NH—(C═O)cyclopropyl, and NH—(C═O)cyclopropyl wherein the cyclopropyl is substituted with one or two halo; 2,5-difluoro-4-pyridyl; 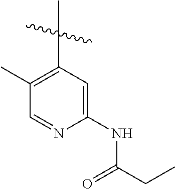 [SMILES: *c1cc(NC(=O)CC)ncc1C]
or 5-methylpyridin-2-amine;
(e) fused heteroaryl selected from the group consisting of: thieno[3,2-b]pyridinyl; pyrazolo[1,5-a]pyridin-5-yl optionally substituted with halo, C₁₋₃alkyl, or NH₂; 1H-pyrrolo[3,2-b]pyridinyl; 1H-pyrrolo[2,3-b]pyridinyl optionally substituted with one two or three members each independently selected from the group consisting of: halo, C₁₋₃alkyl, C₁₋₃haloalkyl, and CN; pyrazolo[1,5-a]pyrimidin-5-yl; [1,2,4]triazolo[1,5-a]pyridin-7-yl optionally substituted with C₁₋₃alkyl; 1H-pyrazolo[4,3-b]pyridin-7-yl optionally substituted with C₁₋₃alkyl; 1H-pyrazolo[3,4-b]pyridin-5-yl optionally substituted with C₁₋₃alkyl; 1H-pyrazolo[3,4-b]pyridinyl optionally substituted with one, two or three members each independently selected from the group consisting of: halo, C₁₋₃alkyl, C₁₋₃haloalkyl, and cyclopropyl; 2-methyl-2H-pyrazolo[4,3-b]pyridin-7-yl; 1-methyl-1H-pyrazolo[4,3-b]pyridin-7-yl; 2-methylpyrazolo[3,4-b]pyridin-4-yl; or 1H-pyrazolo[3,4-d]pyrimidin-4-yl optionally substituted with C₁₋₃alkyl; and
(f) 1,5-naphthyridin-4-yl optionally substituted with halo or OC₁₋₃alkyl;
R³ and R⁴ come together to form a group selected from the group consisting of: 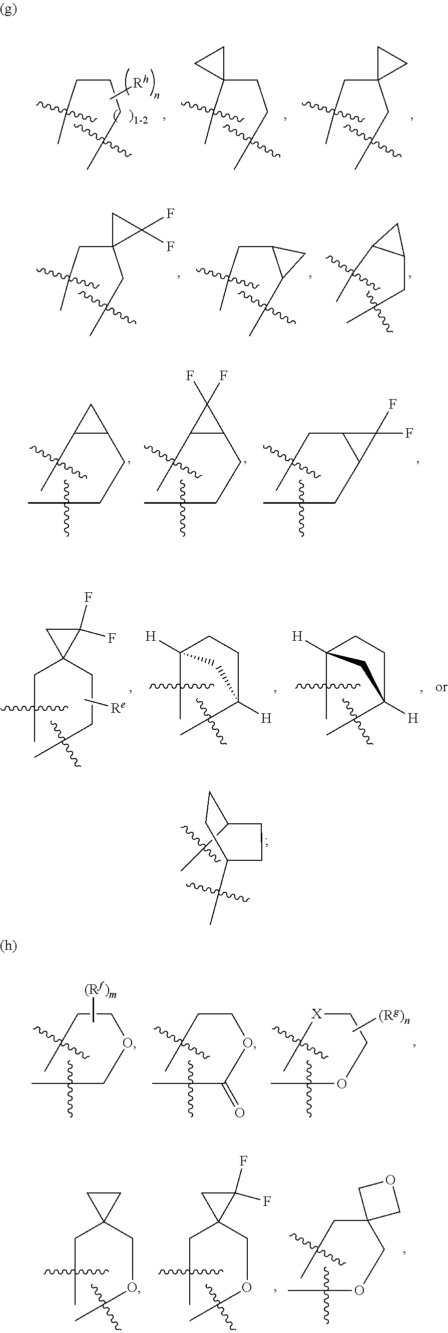 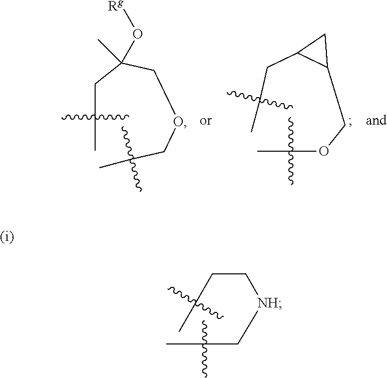
R^{f} is independently selected from the group consisting of: H, C₁₋₃alkyl, C₁₋₃haloalkyl, cyclopropyl, cyclobutyl, and two R^{f} members come together to form a C₃₋₆cycloalkyl wherein the C₃₋₆cycloalkyl is optionally substituted with one or two halo members;
R^{g} is H, halo, or C₁₋₃alkyl;
R^{h} is independently selected from the group consisting of H, halo, OH, C₁₋₃alkyl, CH₂OCH₃, CH₂CH₂OCH₃, C₁₋₃haloalkyl, CH₂OCHF₂, CH₂OCF₃, CN, and 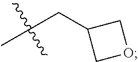 [SMILES: *CC1COC1]
X is selected from the group consisting of: a bond, CH₂, CH(CH₃), and CH₂CH₂;
m is 1, 2, 3 or 4; and
n is 1, 2, or 3;
and pharmaceutically acceptable salts, isotopes, N-oxides, solvates, and stereoisomers of compounds of Formula (I); and
(B) at least one pharmaceutically acceptable excipient.

31. (canceled)

32. A method of treating a subject suffering from or diagnosed with a disease, disorder, or condition mediated by CSNK1D, comprising administering to a subject in need of such treatment a therapeutically effective amount of at least one compound of Formula (I): 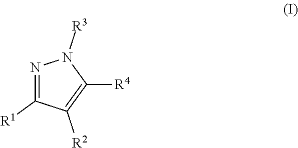
wherein
R¹ is selected from the group consisting of:
(a) phenyl substituted with one or two halo members;
(b) 5-fluoro-2-pyridyl optionally substituted with halo or C₁₋₃alkyl, 5-fluoro-3-pyridyl, 5-chloropyridin-2-yl, 3-chloropyridin-4-yl, 6-methoxypyridin-2-yl, 5-chloro-6-methylpyridin-2-yl, or 3,5-difluoropyridin-4-yl; and
(c) oxazol-5-yl, thiazol-2-yl, thiazol-4-yl, or 1-methyl-1H-imidazol-4-yl;
R² is selected from the group consisting of:
(d) 4-pyridyl optionally substituted with one member selected from the group consisting of: halo, C₁₋₃haloalkyl, CH₂OH, OC₁₋₃alkyl, (C═O)—NHCH₃, NH₂, NH—(C═O)C₁₋₃alkyl, NH—(C═O)C₁₋₃haloalkyl, NH—(C═O)phenyl, NH—(C═O)cyclopropyl, and NH—(C═O)cyclopropyl wherein the cyclopropyl is substituted with one or two halo; 2,5-difluoro-4-pyridyl; 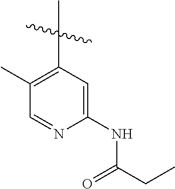 [SMILES: *c1cc(NC(=O)CC)ncc1C]
or 5-methylpyridin-2-amine;
(e) fused heteroaryl selected from the group consisting of: thieno[3,2-b]pyridinyl; pyrazolo[1,5-a]pyridin-5-yl optionally substituted with halo, C₁₋₃alkyl, or NH₂; 1H-pyrrolo[3,2-b]pyridinyl; 1H-pyrrolo[2,3-b]pyridinyl optionally substituted with one two or three members each independently selected from the group consisting of: halo, C₁₋₃alkyl, C₁₋₃haloalkyl, and CN; pyrazolo[1,5-a]pyrimidin-5-yl; [1,2,4]triazolo[1,5-a]pyridin-7-yl optionally substituted with C₁₋₃alkyl; 1H-pyrazolo[4,3-b]pyridin-7-yl optionally substituted with C₁₋₃alkyl; 1H-pyrazolo[3,4-b]pyridin-5-yl optionally substituted with C₁₋₃alkyl; 1H-pyrazolo[3,4-b]pyridinyl optionally substituted with one, two or three members each independently selected from the group consisting of: halo, C₁₋₃alkyl, C₁₋₃haloalkyl, and cyclopropyl; 2-methyl-2H-pyrazolo[4,3-b]pyridin-7-yl; 1-methyl-1H-pyrazolo[4,3-b]pyridin-7-yl; 2-methylpyrazolo[3,4-b]pyridin-4-yl; or 1H-pyrazolo[3,4-d]pyrimidin-4-yl optionally substituted with C₁₋₃alkyl; and
(f) 1,5-naphthyridin-4-yl optionally substituted with halo or OC₁₋₃alkyl;
R³ and R⁴ come together to form a group selected from the group consisting of: 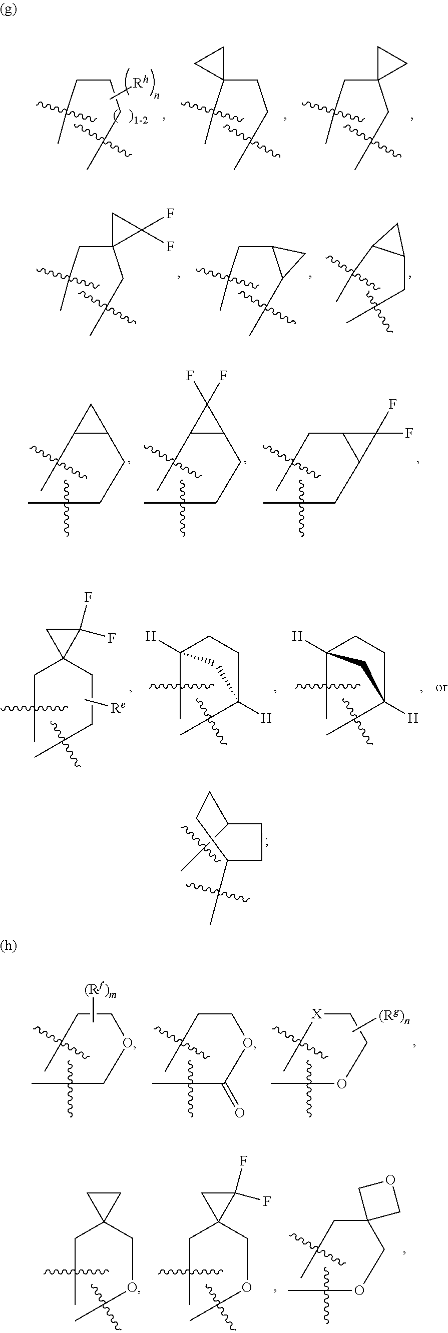 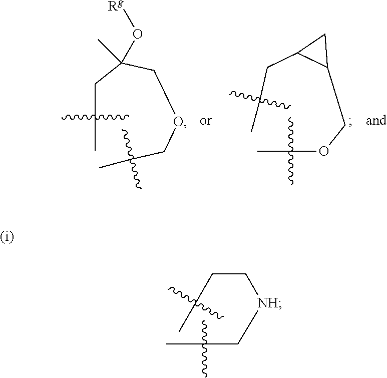
R^{f} is independently selected from the group consisting of: H, C₁₋₃alkyl, C₁₋₃haloalkyl, cyclopropyl, cyclobutyl, and two R^{f} members come together to form a C₃₋₆cycloalkyl wherein the C₃₋₆cycloalkyl is optionally substituted with one or two halo members;
R^{g} is H, halo, or C₁₋₃alkyl;
R^{h} is independently selected from the group consisting of H, halo, OH, C₁₋₃alkyl, CH₂OCH₃, CH₂CH₂OCH₃, C₁₋₃haloalkyl, CH₂OCHF₂, CH₂OCF₃, CN, and 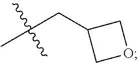 [SMILES: *CC1COC1]
X is selected from the group consisting of: a bond, CH₂, CH(CH₃), and CH₂CH₂;
m is 1, 2, 3 or 4; and
n is 1, 2, or 3;
and pharmaceutically acceptable salts, isotopes, N-oxides, solvates, and stereoisomers of compounds of Formula (I).

33-35. (canceled)
